(19) United States
(12) Patent Application Publication
(10) Pub. No.: US 2023/0301177 A1
(43) Pub. Date: Sep. 21, 2023

(54) COMPOUND, MATERIAL FOR ORGANIC ELECTROLUMINESCENCENT ELEMENT, ORGANIC ELECTROLUMINESCENCENT ELEMENT, AND ELECTRONIC DEVICE

(71) Applicant: IDEMITSU KOSAN CO.,LTD., Tokyo (JP)

(72) Inventors: Takeshi IKEDA, Chiba-shi (JP); Takuto FUKAMI, Chiba-shi (JP); Hiroaki ITOI, Ichikawa-shi (JP); Yusuke TAKAHASHI, Chiba-shi (JP); Shota TANAKA, Chiba-shi (JP); Yongguk LEE, Chiba-shi (JP); Tsukasa SAWATO, Chiba-shi (JP)

(73) Assignee: IDEMITSU KOSAN CO.,LTD., Tokyo (JP)

(21) Appl. No.: 18/183,427

(22) Filed: Mar. 14, 2023

(30) Foreign Application Priority Data
Mar. 18, 2022 (JP) 2022-044131

(51) Int. Cl. H10K 85/60; H10K 50/11; H10K 50/15; H10K 50/805

(52) U.S. Cl. CPC H10K 85/633; H10K 50/11; H10K 50/15; H10K 50/805

(57) ABSTRACT

A compound represented by the following formula (1):

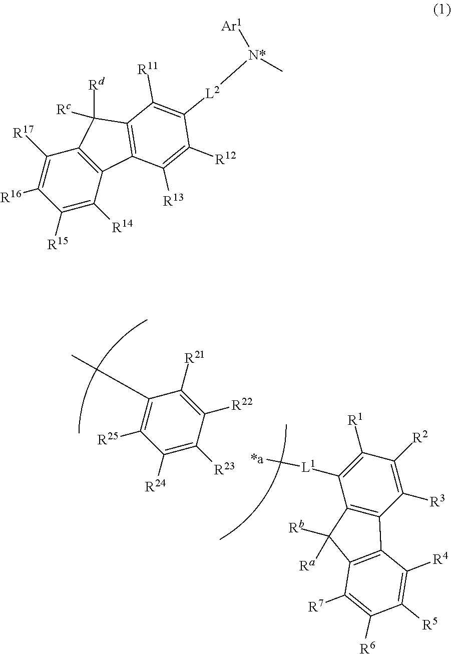

where the symbols in the formula (1) are defined in the description, an organic electroluminescent device containing the compound, and an electronic device including the organic electroluminescent device.

### TECHNICAL FIELD

The present invention relates to a compound, a material for an organic electroluminescent device, an organic electroluminescent device, and an electronic device including the organic electroluminescent device.

### BACKGROUND ART

In general, an organic electroluminescent device (which may be hereinafter referred to as an “organic EL device”) is constituted by an anode, a cathode, and an organic layer intervening between the anode and the cathode. In application of a voltage between both the electrodes, electrons from the cathode side and holes from the anode side are injected into the light emitting region, and the injected electrons and holes are recombined in the light emitting region to generate an excited state, which then returns to the ground state to emit light. Accordingly, development of a material that efficiently transports electrons or holes into the light emitting region, promotes recombination of the electrons and holes, and allows excitons to emit light efficiently, and investigation of the combination thereof are important for providing a high-performance organic EL device.

PTLs 1 to 15 each describe an amine compound having a fluorenyl group bonded directly to the center nitrogen atom, and a fluorenyl group bonded thereto via a phenylene group.

### CITATION LIST

### Patent Literatures

PTL 1: JP 2017-501144 A
PTL 2: US 2018/123042 A
PTL 3: KR 10-2019-0005522 A
PTL 4: KR 10-2019-0114636 A
PTL 5: US 2020/048273 A
PTL 6: CN 108864062 A
PTL 7: KR 10-2018-0096458 A
PTL 8: US 2017/0186978 A
PTL 9: US 2017/0194569 A
PTL 10: US 2017/0186969 A
PTL 11: CN 111333611 A
PTL 12: CN 110317184 A
PTL 13: KR 10-2018-0040079 A
PTL 14: CN 107915648 A
PTL 15: CN 110577510 A

### Technical Problem

Various compounds for organic EL devices have been reported, but the further enhancement of the capability of an organic EL device has been still demanded.

The present invention has been made for solving the problem, and an object thereof is to provide an organic EL device having a further improved device capability with a particular combination of compounds contained therein, and an electronic device including the organic EL device.

### Solution to Problem

As a result of the earnest investigations by the present inventors on the capabilities of organic EL devices containing the compounds described in PTLs 1 to 15, it has been found that an organic EL device including a compound represented by the following formula (1) has a further improved capability.

In one embodiment, the present invention provides a compound represented by the following formula (1):

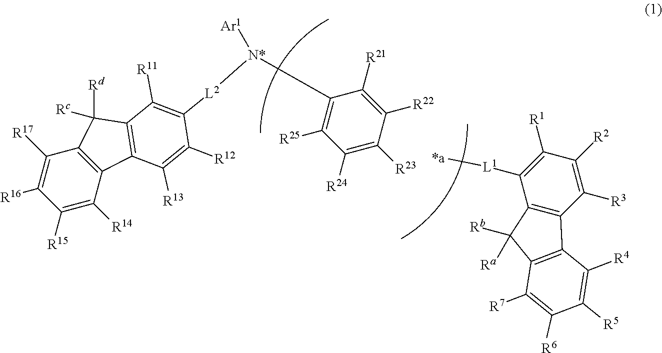

wherein in the formula (1),
N* represents a center nitrogen atom,
L¹ and L² each independently represent a single bond or a substituted or unsubstituted phenylene group, in which the substituent is an unsubstituted alkyl group having 1 to 10 carbon atoms, and no ring is condensed to the phenylene group,
R¹ to R⁷ and R¹¹ to R¹⁷ each independently represent a hydrogen atom or an unsubstituted alkyl group having 1 to 10 carbon atoms,
adjacent two selected from R¹ to R⁷ and R¹¹ to R¹⁷ are not bonded to each other, and therefore do not form a ring,
R^{a} and R^{b} each independently represent a hydrogen atom or a substituted or unsubstituted alkyl group having 1 to 30 carbon atoms,
R^{a} and R^{b} may be bonded to each other to form a substituted or unsubstituted ring,
R^{a} and R^{d} each independently represent a hydrogen atom, a substituted or unsubstituted alkyl group having 1 to 30 carbon atoms, or a substituted or unsubstituted aryl group having 6 to 30 ring carbon atoms,
R^{c} and R^{d} may be bonded to each other to form a substituted or unsubstituted ring,
provided that in the case where R^{c} and R^{d} are not bonded to each other not to form a substituted or unsubstituted ring, at least one of R^{c} and R^{d} represents a substituted or unsubstituted alkyl group having 1 to 30 carbon atoms,
R^{c} and R^{d} are not bonded to each other not to form a substituted or unsubstituted fluorene ring with a carbon atom at the 9-position of the fluorene skeleton,
one selected from R²¹ to R²⁵ represents a single bond bonded to *a, and R²¹ to R²⁵ that are not the single bond bonded to *a represent hydrogen atoms, and
Ar¹ represents a group represented by any of the following formulae (1a) to (1f):

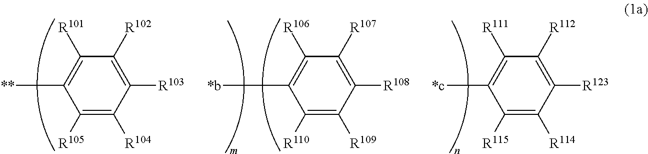

wherein in the formula (1a),
** represents a bonding site to the center nitrogen atom N*,
R¹⁰¹ to R¹⁰⁵ and R¹⁰⁶ to R¹¹⁰ each independently represent a hydrogen atom or an unsubstituted alkyl group having 1 to 6 carbon atoms,
provided that one selected from R¹⁰¹ to R¹⁰⁶ represents a single bond bonded to *b, and one selected from R¹⁰⁶ to R¹¹⁰ represents a single bond bonded to *c,
adjacent two selected from R¹⁰¹ to R¹⁰⁵ that are not the single bond are not bonded to each other, and therefore do not form a ring, and
adjacent two selected from R¹⁰⁶ to R¹¹⁰ that are not the single bond are not bonded to each other, and therefore do not form a ring,
m represents 0, 1, or 2, n represents 0, 1, or 2,
in which m+n is 0, 2, or 3,
R¹¹¹ to R¹¹⁵ each independently represent a hydrogen atom, a substituted or unsubstituted alkyl group having 1 to 6 carbon atoms, or a substituted or unsubstituted heteroaryl group having 5 to 13 ring atoms, and
adjacent two selected from R¹¹¹ to R¹¹⁵ may be bonded to each other to form one or multiple unsubstituted benzene rings, or may not be bonded to each other, and therefore may not form a ring,

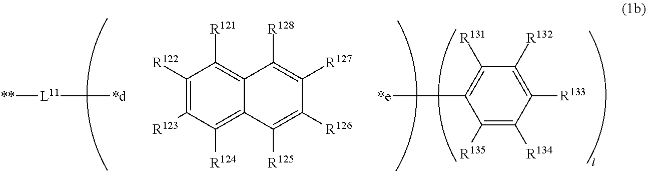

wherein in the formula (1b),
** represents a bonding site to the center nitrogen atom N*,
Ln represents a single bond, an unsubstituted arylene group having 6 to 12 ring carbon atoms, or an unsubstituted divalent heterocyclic group having 5 to 13 ring atoms,
R¹²¹ to R¹²⁸ each independently represent a hydrogen atom, a substituted or unsubstituted alkyl group having 1 to 6 carbon atoms, or a substituted or unsubstituted aryl group having 6 to 12 ring carbon atoms,
provided that one selected from R¹²¹ to R¹²⁸ represents a single bond bonded to *d, the other one selected from R¹²¹ to R¹²⁸ represents a single bond bonded to *e, and
adjacent two selected from R¹²¹ to R¹²⁸ that are not the single bond bonded to *d and a single bond bonded to *e are not bonded to each other, and therefore do not form a ring,
R¹³¹ to R¹³⁵ each independently represent a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
adjacent two selected from R¹³¹ to R¹³⁵ may be bonded to each other to form one or multiple unsubstituted benzene rings, or may not be bonded to each other, and therefore may not form a ring, and
l represents 0 or 1,

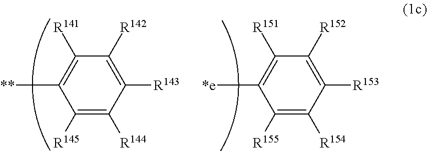

wherein in the formula (1c),
** represents a bonding site to the center nitrogen atom N*,
R¹⁴¹ to R¹⁴ each independently represent a hydrogen atom or a substituted or unsubstituted alkyl group having 1 to 6 carbon atoms,
provided that one selected from R¹⁴¹, R¹⁴², R¹⁴⁴, and R¹⁴⁵ represents a single bond bonded to *e, and
adjacent two selected from R¹⁴³, and R¹⁴¹, R¹⁴², R¹⁴⁴, and R¹⁴⁵ that are not the single bond bonded to *e are not bonded to each other, and therefore do not form a ring,
R¹⁵¹ to R¹⁵⁵ each independently represent a hydrogen atom or a substituted or unsubstituted alkyl group having 1 to 6 carbon atoms, and
adjacent two selected from R¹⁵¹ to R¹⁵⁶ are not bonded to each other, and therefore do not form a ring,

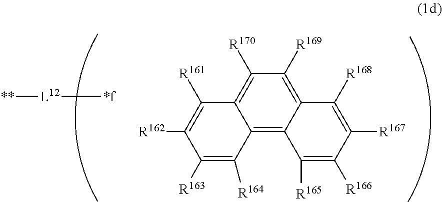

wherein in the formula (1d),
** represents a bonding site to the center nitrogen atom N*,
L¹² represents a single bond, an unsubstituted arylene group having 6 to 12 ring carbon atoms, or an unsubstituted divalent heterocyclic group having 5 to 13 ring atoms,
R¹⁶¹ to R¹⁷⁰ each independently represent a hydrogen atom, a substituted or unsubstituted alkyl group having 1 to 6 carbon atoms, or a substituted or unsubstituted aryl group having 6 to 12 ring carbon atoms,
provided that in the case where L¹² represents a single bond, one selected from R¹⁶¹ to R¹⁶⁸ represents a single bond bonded to *f, and in the case where L¹² represents an unsubstituted arylene group having 6 to 12 ring carbon atoms or an unsubstituted divalent heterocyclic group having 5 to 13 ring atoms, one selected from R¹⁶¹ to R¹⁷⁰ represents a single bond bonded to *f, and
adjacent two selected from R¹⁶¹ to R¹⁷⁰ that are not the single bond are not bonded to each other, and therefore do not form a ring,

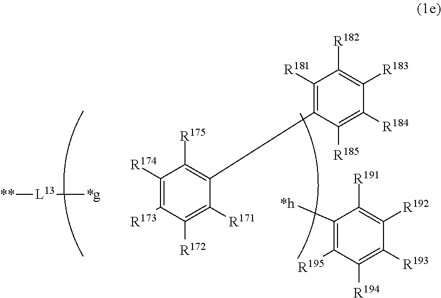

wherein in the formula (1e),
** represents a bonding site to the center nitrogen atom N*,
L¹³ represents a single bond, an unsubstituted arylene group having 6 to 12 ring carbon atoms, or an unsubstituted divalent heterocyclic group having 5 to 13 ring atoms,
R¹⁷¹ to R¹⁷⁵ each independently represent a hydrogen atom, an unsubstituted alkyl group having 1 to 6 carbon atoms, or an unsubstituted phenyl group,
provided that one selected from R¹⁷¹ to R¹⁷⁵ represents a single bond bonded to *g, and the other one selected from R¹⁷¹ and R¹⁷² represents a single bond bonded to *h, and
adjacent two selected from R¹⁷¹ to R¹⁷⁵ that are not the single bond bonded to *g and the single bond bonded to *h are not bonded to each other, and therefore do not form a ring,
R¹⁸¹ to R¹⁸⁵ and R¹⁹¹ to R¹⁹⁶ each independently represent a hydrogen atom or an unsubstituted alkyl group having 1 to 6 carbon atoms, and
adjacent two selected from R¹⁸¹ to R¹⁸⁵ and R¹⁹¹ to R¹⁹⁶ may be bonded to each other to form one or multiple unsubstituted benzene rings, or may not be bonded to each other, and therefore may not form a ring,

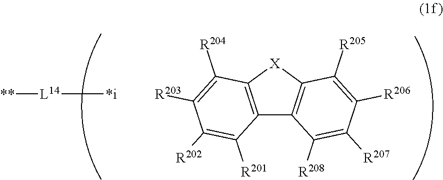

wherein in the formula (1f),
** represents a bonding site to the center nitrogen atom N*,
L¹⁴ represents a single bond or an unsubstituted phenylene group,
X represents an oxygen atom, a sulfur atom, or CR^{A}R^{B},
wherein R^{A} and R^{B} each independently represent a hydrogen atom, a substituted or unsubstituted alkyl group having 1 to 30 carbon atoms, or a substituted or unsubstituted aryl group having 6 to 30 ring carbon atoms, and
R^{A} and R^{B} may be bonded to each other to form a substituted or unsubstituted ring, or may not be bonded to each other, and therefore may not form a ring, and
R²⁰¹ to R²⁰⁸ each independently represent a hydrogen atom, a substituted or unsubstituted alkyl group having 1 to 6 carbon atoms, or a substituted or unsubstituted aryl group having 6 to 12 ring carbon atoms,
provided that in the case where L¹⁴ represents a phenylene group, and X represents an oxygen atom, one selected from R²⁰¹ to R²⁰³, R²⁰⁶ to R²⁰⁸, R^{A}, and R^{B} represents a single bond bonded to *i, and
in the case where L¹⁴ represents a single bond, and X represents an oxygen atom, a sulfur atom, or CR^{A}R^{B}, and in the case where L¹⁴ represents a phenylene group, and X represents a sulfur atom or CR^{A}R^{B}, one selected from R²⁰¹ to R²⁰⁸, R^{A}, and R^{B} represents a single bond bonded to *i,
in the case where X represents CR^{A}R^{B}, adjacent two selected from R²⁰¹ to R²⁰⁸ that are not the single bond are not bonded to each other, and therefore do not form a ring, and
in the case where X represents an oxygen atom or a sulfur atom, adjacent two selected from R²⁰¹ to R²⁰⁸ that are not the single bond may be bonded to each other to form one or multiple unsubstituted benzene rings, or may not be bonded to each other, and therefore may not form a ring.

In another embodiment, the present invention provides a material for an organic EL device, containing the compound represented by the formula (1).

In still another embodiment, the present invention provides an organic electroluminescent device including an anode, a cathode, and organic layers intervening between the anode and the cathode, the organic layers including a light emitting layer, at least one layer of the organic layers containing the compound represented by the formula (1).

In further another embodiment, the present invention provides an electronic device including the organic electroluminescent device.

### Advantageous Effects of Invention

An organic EL device containing the compound represented by the formula (1) shows an improved device capability.

### BRIEF DESCRIPTION OF DRAWINGS

FIG. 1 is a schematic illustration showing an example of the layer configuration of the organic EL device according to one embodiment of the present invention.

FIG. 2 is a schematic illustration showing another example of the layer configuration of the organic EL device according to one embodiment of the present invention.

FIG. 3 is a schematic illustration showing still another example of the layer configuration of the organic EL device according to one embodiment of the present invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocycic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenyfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A):

a phenyl group,
a p-biphenylyl group,
a m-biphenylyl group,
an o-biphenylyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

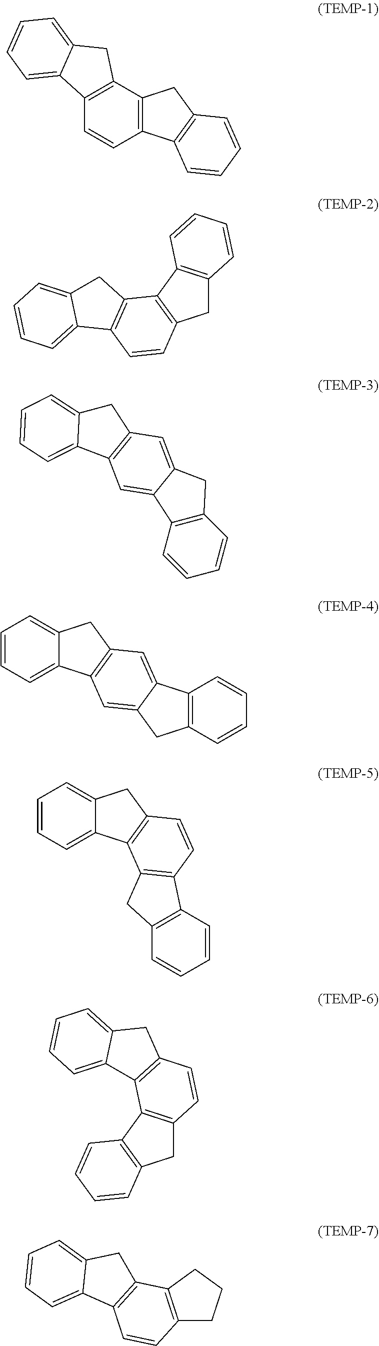

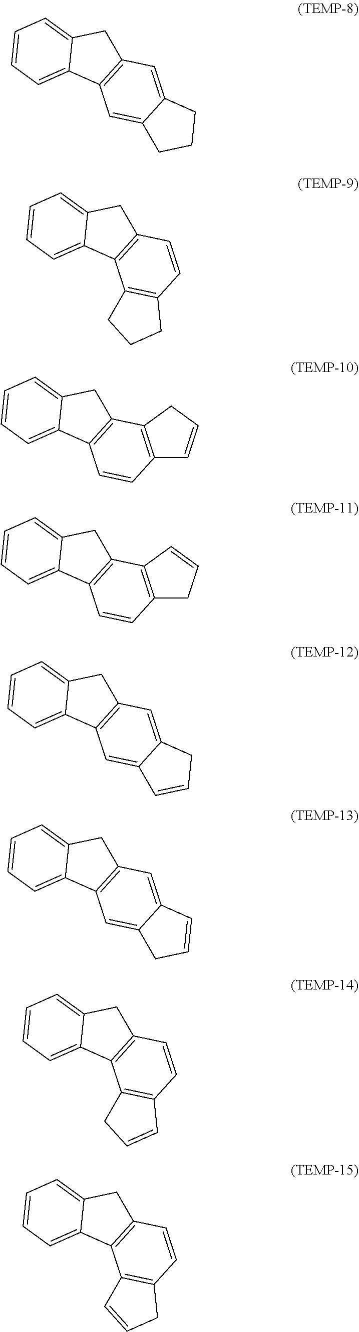

### Substituted Aryl Group (Set of Specific Examples G1B):

an o-tolyl group,
a m-tolyl group,
a p-tolyl group,
a p-xylyl group,
a m-xylyl group,
an o-oylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilyiphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMIP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

Unsubstituted Heterocyclic Group containing Nitrogen Atom (Set of Specific Examples G2A1):
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

Unsubstituted Heterocyclic Group containing Oxygen Atom (Set of Specific Examples G2A2):
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

Unsubstituted Heterocyclic Group containing Sulfur Atom (Set of Specific Examples G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
   Monovalent Heterocyclic Group Derived by Removing One Hydrogen Atom from Ring Structures Represented by General Formulae (TEMP-16) to (TEMP-33) (Set of Specific Examples G2A4)

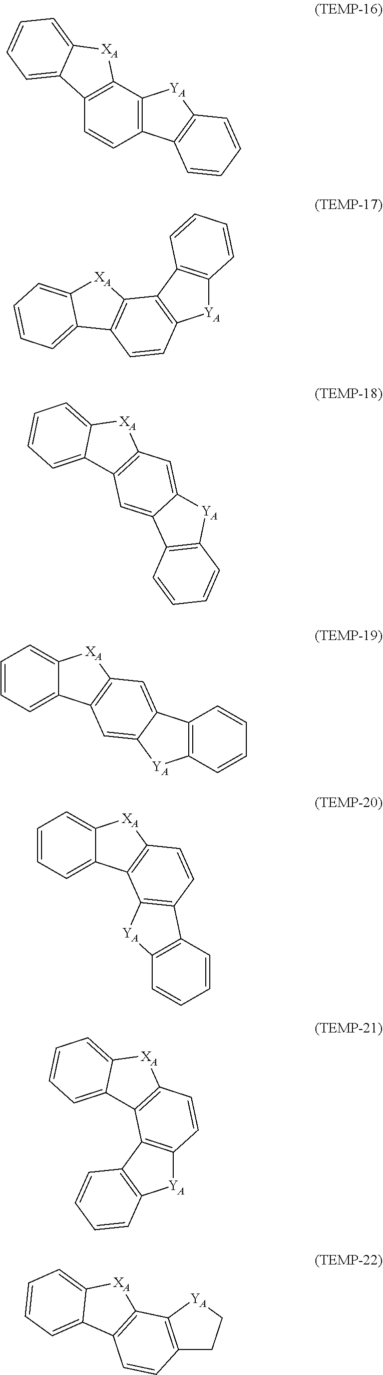

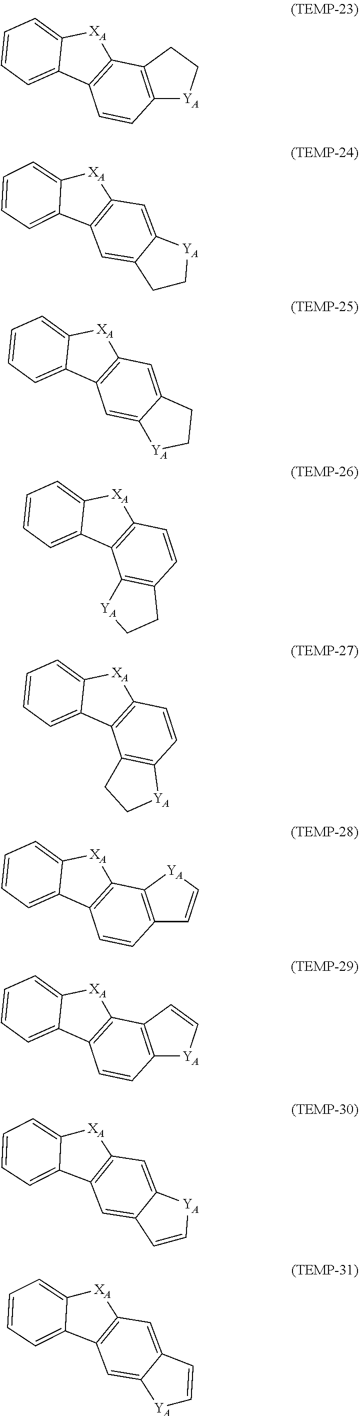

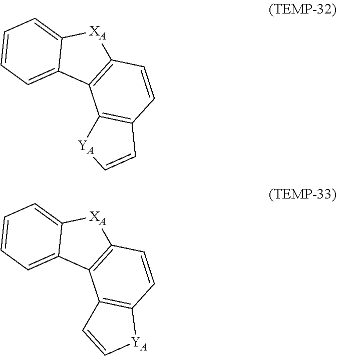

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

### Substituted Heterocyclic Group Containing Nitrogen Atom (Set of Specific Examples G2B1):

a (9-phenyl)carbazolyl group,
a (9-biphenylyl)carbazolyl group,
a (9-phenyl)phenylcarbazolyl group.
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and
a biphenylquinazolinyl group.

### Substituted Heterocyclic Group Containing Oxygen Atom (Set of Specific Examples G2B2):

a phenyldibenzofuranyl group,
a methyldibenzofuranyl group,
a t-butyldibenzofuranyl group, and
a monovalent residual group of spiro[9H-xanthene-9,9′-[9H]fluorene].

### Substituted Heterocyclic Group Containing Sulfur Atom (Set of Specific Examples G2B3):

a phenyldibenzothiophenyl group,
a methyldibenzothiophenyl group,
a t-butyldibenzothiophenyl group, and
a monovalent residual group of spiro[9H-thioxanthene-9,9′-[9H]fluorene].
   Group Formed by Substituting One or More Hydrogen Atom of Monovalent Heterocyclic Group Derived from Ring Structures Represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A):

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B):

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A):

a vinyl group,
an allyl group,
a 1-butenyl group.
a 2-butenyl group, and
a 3-butenyl group.

### Substituted Alkenyl Group (Set of Specific Examples G4B):

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

### Unsubstituted Alkynyl Group (Set of Specific Examples G5A):

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Set of Specific Examples G6B):

a 4-methylcyclohexyl group.
   Group represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

Group represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group represented by —N(R₉₀₆)(R₉₀₇)

In the description herein, specific examples (set of specific examples 010) of the group represented by —N(R₉₀₆)(R₉₀₇) include:
—N(G1)(G1),
—N(G2)(G2),
—N(G1)(G2),
—N(G3)(G3), and
—N(G6)(G6).
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-8-naphthylethyl group, a 1-8-naphthylisopropyl group, and a 2-8-naphthylisopropyl group.

In the description herein, the substituted or unsubstituted aryl group is preferably a phenyl group, a p-biphenylyl group, a m-biphenylyl group, an o-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranly group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

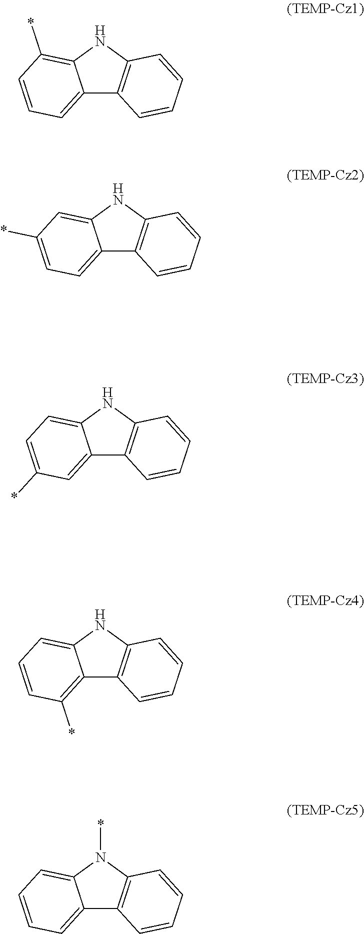 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

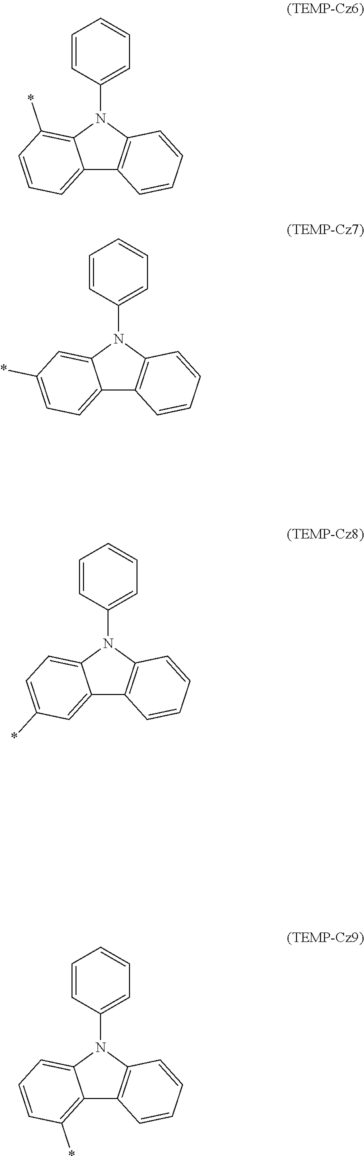 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

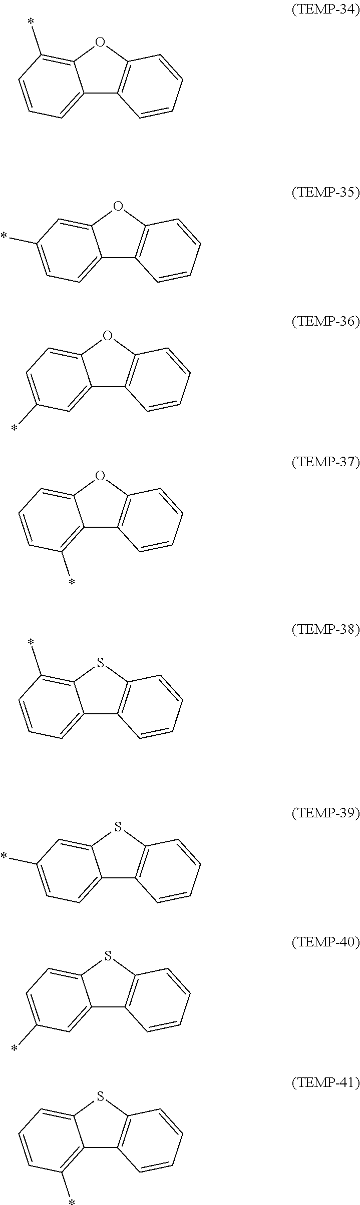

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

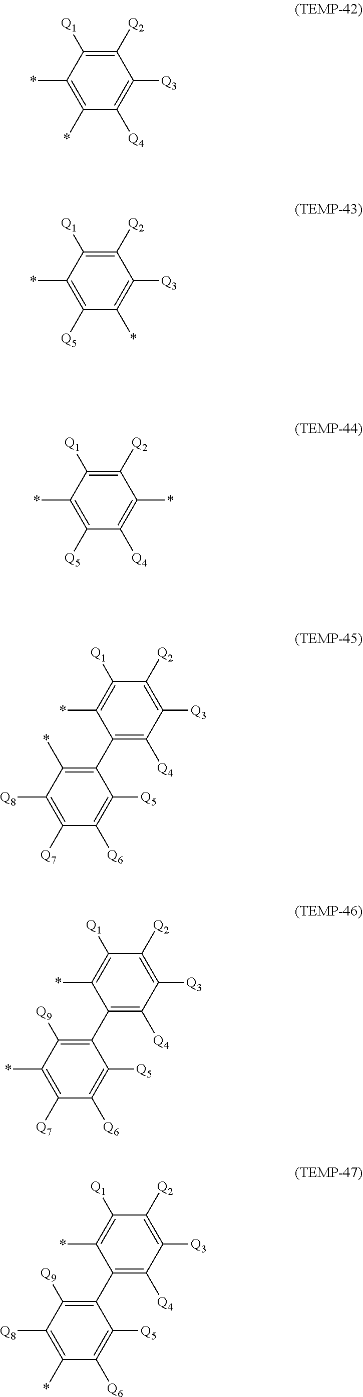

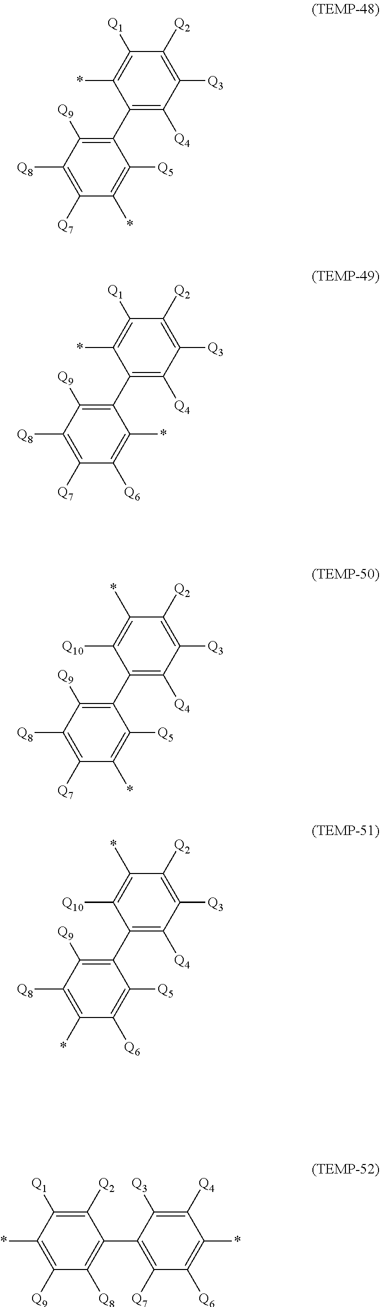

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

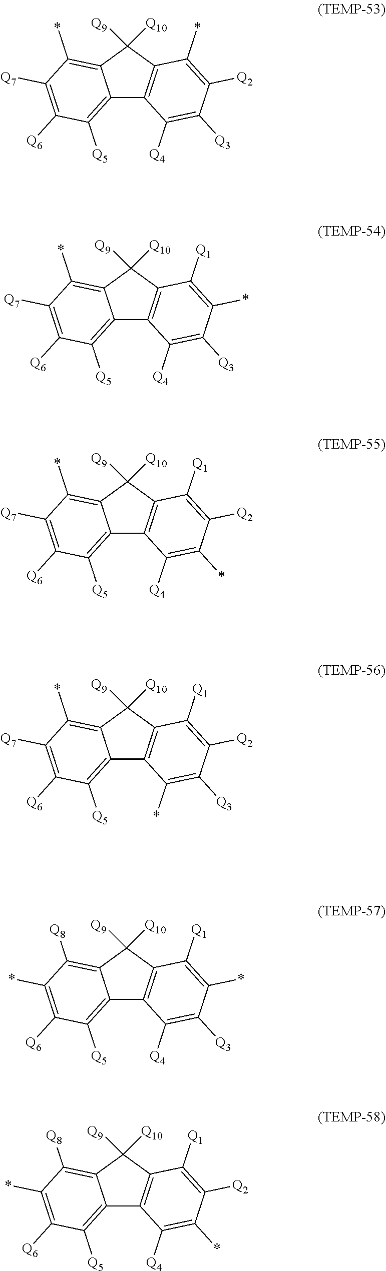

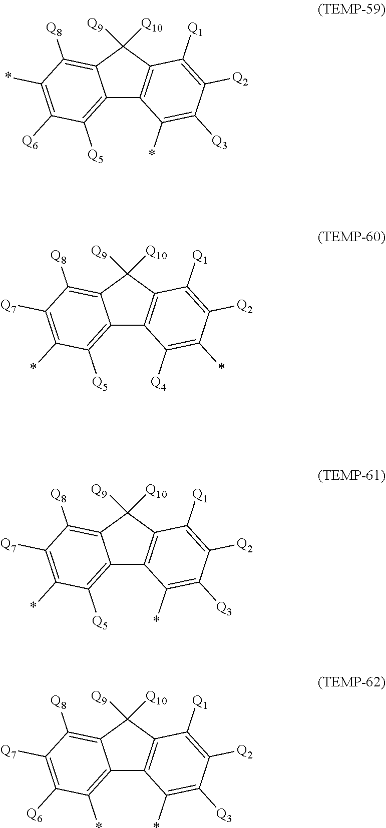

In the general formulae (TEMP-53) to (TEMP-62). Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

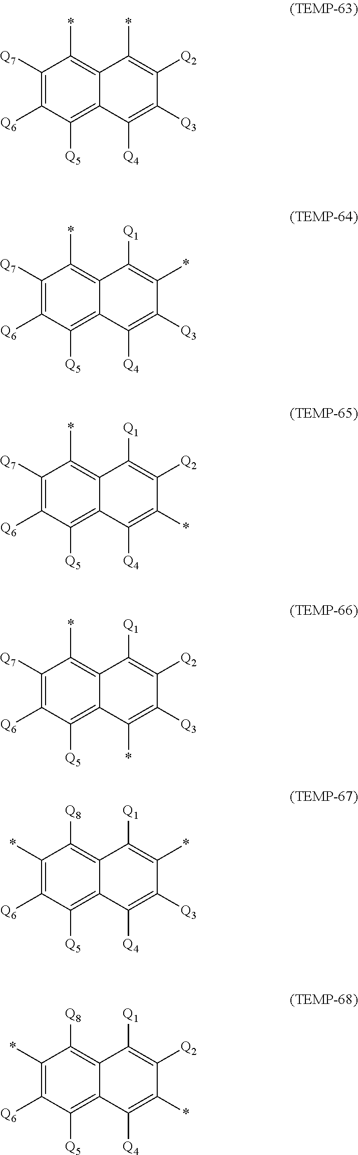

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

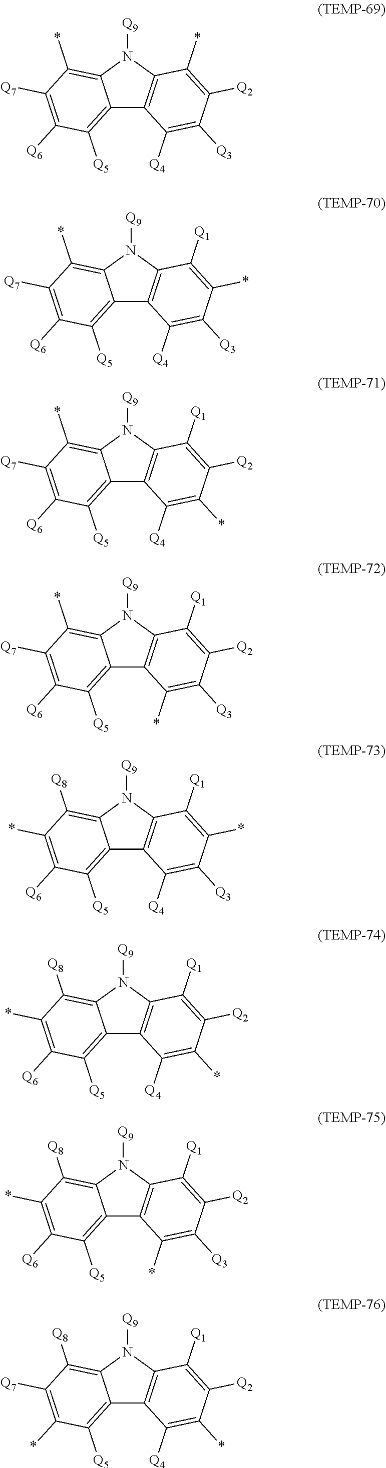

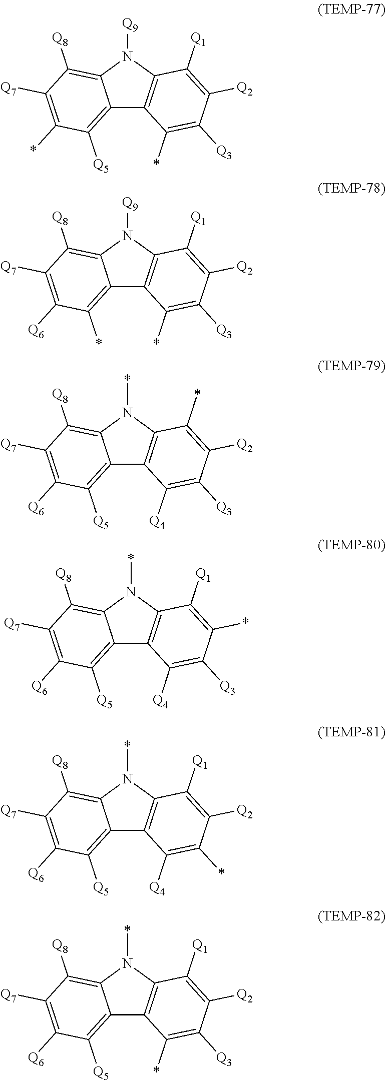

In the general formulae (TEMP-69) to (TEMP-82), Q₁ to Q₉ each independently represent a hydrogen atom or a substituent.

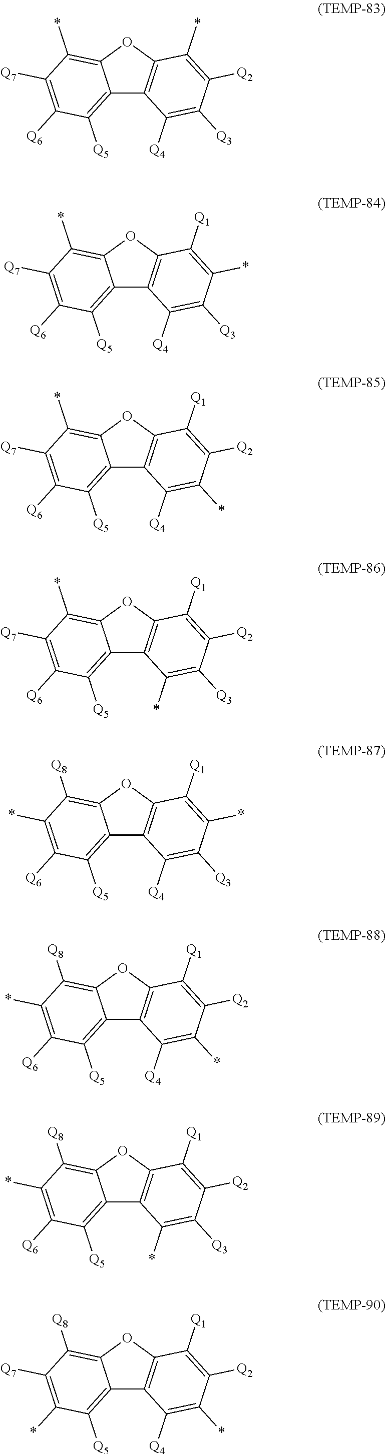

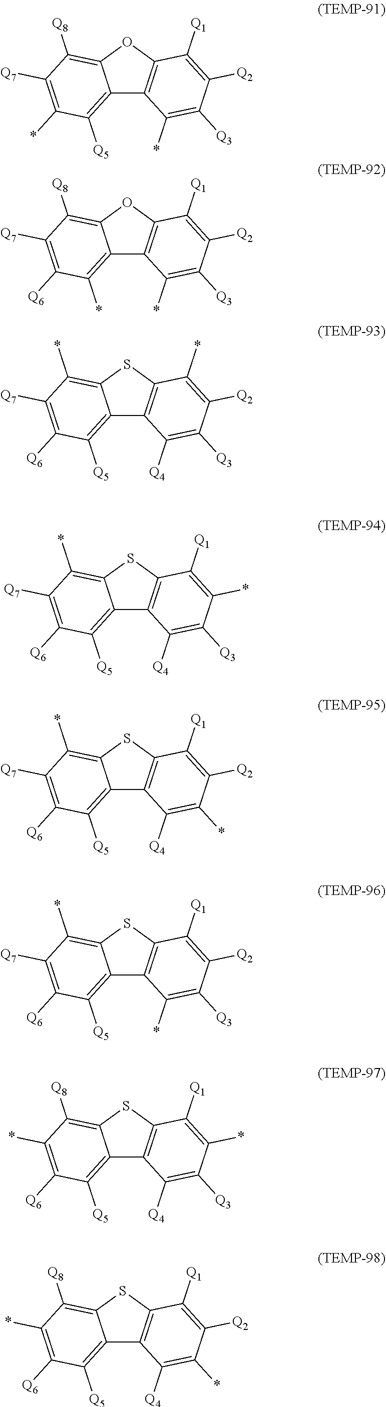

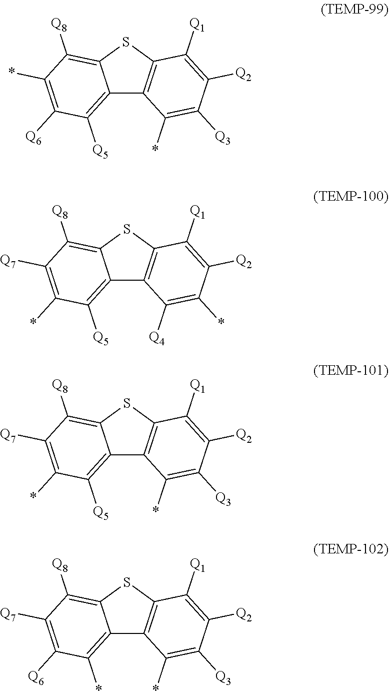

In the general formulae (TEMP-83) to (TEMP-102), Q₁ to Q₈ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

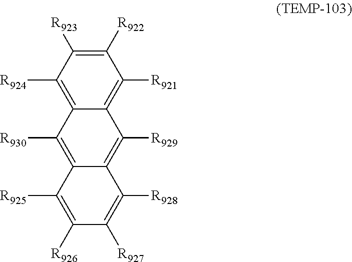

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

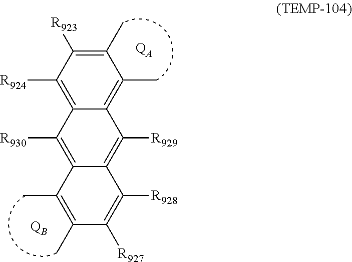

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Q_{C}, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

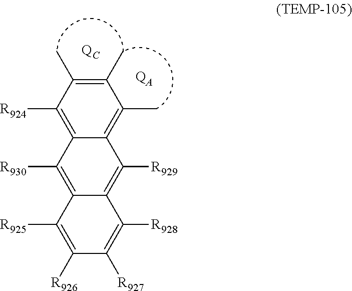

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or a non-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

The compound of the present invention will be described below.

The compound of the present invention is represented by the formula (1). The symbols in the formula (1) and the formulae included in the formula (1) will be described. The same symbols have the same meaning unless otherwise indicated.

The compounds of the present invention represented by the formula (1) each may be referred simply to as an “inventive compound”.

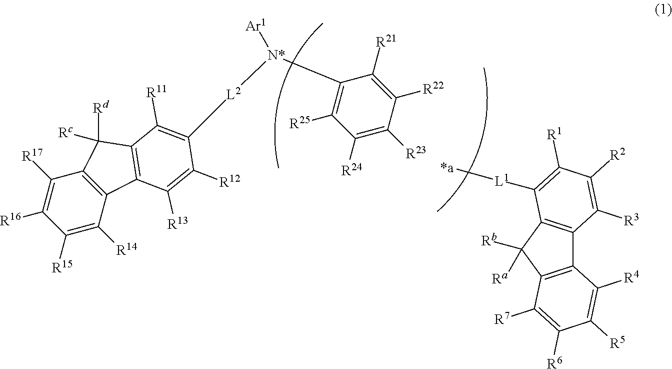

N* represents a center nitrogen atom.

L¹ and L² each independently represent a single bond or a substituted or unsubstituted phenylene group, in which the substituent is an unsubstituted alkyl group having 1 to 10 carbon atoms, and no ring is condensed to the phenylene group.

L¹ and L² each preferably represent a single bond.

The substituted or unsubstituted phenylene group is preferably an unsubstituted phenylene group.

The phenylene group is an o-phenylene group, a m-phenylene group, or a p-phenylene group, and preferably a p-phenylene group.

The unsubstituted alkyl group having 1 to 10 carbon atoms is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and further preferably a methyl group or a t-butyl group.

In one embodiment of the present invention, one or both of L¹ and L² preferably represents a single bond.

R¹ to R⁷ and R¹¹ to R¹⁷ each independently represent a hydrogen atom or an unsubstituted alkyl group having 1 to 10, and preferably 1 to 6, carbon atoms.

Adjacent two selected from R¹ to R⁷ and R¹¹ to R¹⁷ are not bonded to each other, and therefore do not form a ring.

The unsubstituted alkyl group having 1 to 10 carbon atoms is, for example,
a methyl group, an ethyl group, a n-propyl group, an isopropyl group, a n-butyl group, an isobutyl group, a s-butyl group, a t-butyl group, a pentyl group, a hexyl group, a heptyl group, an octyl group, a nonyl group, or a decyl group;
preferably a methyl group, an ethyl group, a n-propyl group, an isopropyl group, a n-butyl group, an isobutyl group, a s-butyl group, a t-butyl group, or a pentyl group;
more preferably a methyl group, an ethyl group, a n-propyl group, an isopropyl group, a n-butyl group, an isobutyl group, a s-butyl group, or a t-butyl group; and
further preferably a methyl group or a t-butyl group.

All R¹ to R⁷ and R¹¹ to R¹⁷ may represent hydrogen atoms.

R^{a} and R^{b} each independently represent a hydrogen atom or a substituted or unsubstituted alkyl group having 1 to 30, preferably 1 to 18, and more preferably 1 to 6, carbon atoms.

R^{a} and R^{b} may be bonded to each other to form a substituted or unsubstituted ring.

The unsubstituted alkyl group having 1 to 30 carbon atoms is, for example,
a methyl group, an ethyl group, a n-propyl group, an isopropyl group, a n-butyl group, an isobutyl group, a s-butyl group, a t-butyl group, a pentyl group, a hexyl group, a heptyl group, an octyl group, a nonyl group, a decyl group, an undecyl group, or a dodecyl group;
preferably a methyl group, an ethyl group, a n-propyl group, an isopropyl group, a n-butyl group, an isobutyl group, a s-butyl group, a t-butyl group, or a pentyl group;
more preferably a methyl group, an ethyl group, a n-propyl group, an isopropyl group, a n-butyl group, an isobutyl group, a s-butyl group, or a t-butyl group; and
further preferably a methyl group or a t-butyl group.

In one embodiment of the present invention, at least one of R^{a} and R^{b} preferably represents a substituted or unsubstituted alkyl group having 1 to 30 carbon atoms.

In one embodiment of the present invention, R^{a} and R^{b} each preferably represent a methyl group or a t-butyl group, and more preferably a methyl group.

The unsubstituted monocyclic ring formed by R^{a} and R^{b} is, for example, a benzene ring, a cyclopentane ring, and a cyclohexane ring.

The unsubstituted condensed ring formed by R^{a} and R^{b} is, for example, a naphthalene ring and an anthracene ring.

In the case where R^{a} and R^{b} are bonded to each other to form an unsubstituted monocyclic ring or an unsubstituted condensed ring, R^{a} and R^{b} may form a ring with the fluorene skeleton, to which these groups are bonded, and may form a spiro ring. The spiro ring may be a hydrocarbon ring or a heterocyclic ring, and may contain a ring selected from a monocyclic ring, a condensed ring, a bridged bicyclo ring, and a bridged tricyclo ring. Examples of the substituted or unsubstituted spiro ring are shown below, but are not limited thereto. * represents a bonding site to the benzene ring of the fluorene skeleton.

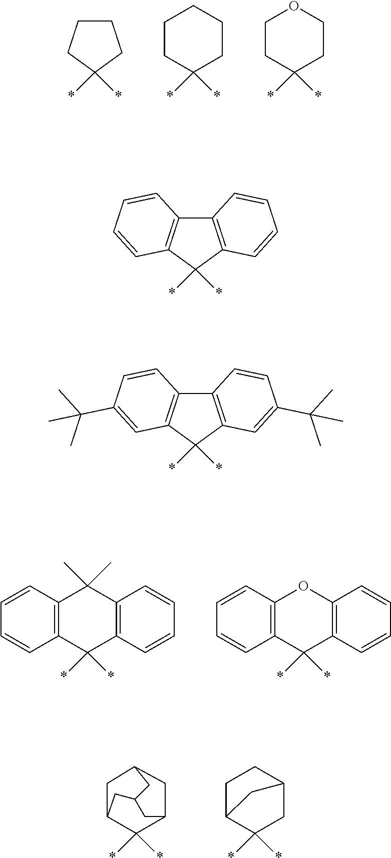

R^{c} and R^{d} each independently represent a hydrogen atom, a substituted or unsubstituted alkyl group having 1 to 30, preferably 1 to 18, and more preferably 1 to 6, carbon atoms, or a substituted or unsubstituted aryl group having 6 to 30, preferably 6 to 25, and more preferably 6 to 12, ring carbon atoms.

R^{c} and R^{d} may be bonded to each other to form a substituted or unsubstituted ring.

In the case where R^{c} and R^{d} are not bonded to each other not to form a substituted or unsubstituted ring, at least one of R^{c} and R^{d} represents a substituted or unsubstituted alkyl group having 1 to 30 carbon atoms, and

R^{c} and R^{d} are not bonded to each other not to form a substituted or unsubstituted fluorene ring with a carbon atom at the 9-position of the fluorene skeleton.

The details of the substituted or unsubstituted alkyl group having 1 to 30 carbon atoms and the substituted or unsubstituted ring have been described for R^{a} and R^{b}.

The unsubstituted aryl group having 6 to 30 ring carbon atoms is, for example,
a phenyl group, a biphenylyl group, a terphenylyl group, a biphenylenyl group, a naphthyl group, an anthryl group, a benzanthryl group, a phenanthryl group, a benzophenanthryl group, a phenalenyl group, a picenyl group, a pentaphenyl group, a pyrenyl group, a chrysenyl group, a benzocrysenyl group, a fluorenyl group, a fluoranthenyl group, a perylenyl group, or a triphenylenyl group;
preferably a phenyl group, a biphenylyl group, a terphenylyl group, or a naphthyl group;
more preferably a phenyl group, a 2-, 3-, or 4-biphenylyl group, a 2-, 3-, or 4-o-terphenylyl group, a 2-, 3-, or 4-m-terphenylyl group, a 2-, 3-, or 4-p-terphenylyl group, or a 1- or 2-naphthyl group;
further preferably a phenyl group, a 2-, 3-, or 4-biphenylyl group, or a 1- or 2-naphthyl group; and
particularly preferably a phenyl group.

The unsubstituted monocyclic ring represented by R^{c} and R^{d} is, for example, a benzene ring, a cyclopentane ring, or a cyclohexane ring.

The unsubstituted condensed ring represented by R^{c} and R^{d} is, for example, a naphthalene ring or an anthracene ring.

In the case where R^{c} and R^{d} are bonded to each other to form an unsubstituted monocyclic ring or an unsubstituted condensed ring, R^{c} and R^{d} may form a ring with the fluorene skeleton, to which these groups are bonded, and may form a spiro ring. The spiro ring may be a hydrocarbon ring or a heterocyclic ring, and may contain a ring selected from a monocyclic ring, a condensed ring, a bridged bicyclo ring, and a bridged tricyclo ring. R^{c} and R^{d} are not bonded to each other not to form a substituted or unsubstituted fluorene ring with a carbon atom at the 9-position of the fluorene skeleton. Accordingly, R^{c} and R^{d} do not form a substituted or unsubstituted fluorene ring, which is a ring completing a 9,9′-spirobifluorene structure, and do not form the following spiro ring structures. * represents a bonding site to the benzene ring of the fluorene skeleton.

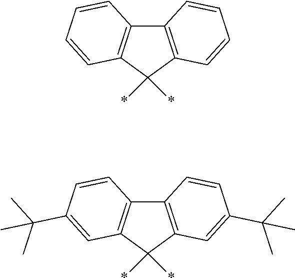 [SMILES: *C1(*)c2ccccc2-c2ccccc21 ; *C1(*)c2cc(C(C)(C)C)ccc2-c2ccc(C(C)(C)C)cc21]

In one embodiment of the present invention, it is preferred that R^{c} and R^{d} are bonded to each other to form a substituted or unsubstituted spiro ring with a carbon atom at the 9-position of the fluorene skeleton, and the spiro ring is selected from the following. * represents a bonding site to the benzene ring of the fluorene skeleton.

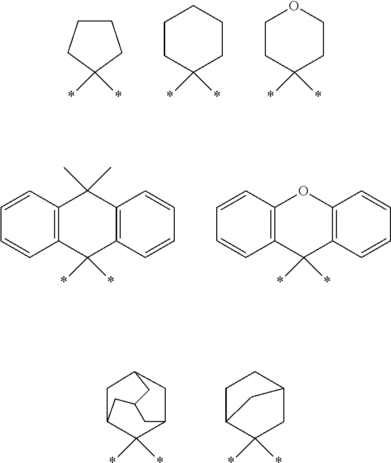

In one embodiment of the present invention, R^{c} and R^{d} each preferably represent a substituted or unsubstituted alkyl group having 1 to 30 carbon atoms, more preferably a methyl group, an ethyl group, a n-propyl group, an isopropyl group, a n-butyl group, an isobutyl group, a s-butyl group, or a t-butyl group, more preferably a methyl group or a t-butyl group, and further preferably a methyl group.

In one embodiment of the present invention, R^{c} and R^{d} are not bonded to each other, and therefore do not form a ring.

One selected from R²¹ to R²⁵, preferably one selected from R²² to R²⁴, and more preferably R²³, preferably represents a single bond bonded to *a, and R²¹ to R²⁵ that are not the single bond bonded to *a represent hydrogen atoms.

Ar¹ represents a group represented by any of the following formulae (1a) to (1f).

The formula (1a) is represented by the following formula.

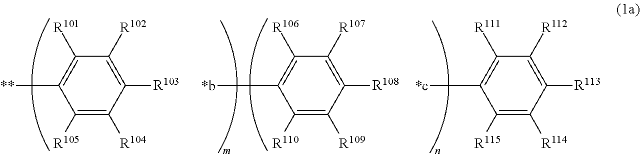

In the formula (1a),
** represents a bonding site to the center nitrogen atom N*.

R¹⁰¹ to R¹⁰⁵ and R¹⁰⁶ to R¹¹⁰ each independently represent a hydrogen atom or an unsubstituted alkyl group having 1 to 6 carbon atoms.

One selected from R¹⁰¹ to R¹⁰⁵ represents a single bond bonded to *b, and one selected from R¹ to R¹¹⁰ represents a single bond bonded to *c.

The details of the unsubstituted alkyl group having 1 to 6 carbon atoms have been described for R^{a} and R^{b} except that the number of carbon atoms thereof is 1 to 6.

Adjacent two selected from R¹⁰¹ to R¹⁰⁵ that are not the single bond are not bonded to each other, and therefore do not form a ring.

Adjacent two selected from R¹⁰⁶ to R¹¹⁰ that are not the single bond are not bonded to each other, and therefore do not form a ring.

All R¹⁰¹ to R¹⁰⁵ that are not the single bond are not bonded may represent hydrogen atoms, and all R¹⁰⁶ to R¹¹⁰ that are not the single bond may represent hydrogen atoms.
m represents 0, 1, or 2, n represents 0, 1, or 2,
in which m+n is 0, 2, or 3.

In one embodiment of the present invention, m represents 0, and n represents 0. In this case, *c represents **, and the formula (1a) is represented by the following formula.

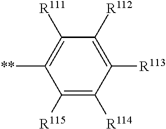

In another embodiment of the present invention, m represents 1, and n represents 1. In this case, the formula (1a) is represented by the following formula.

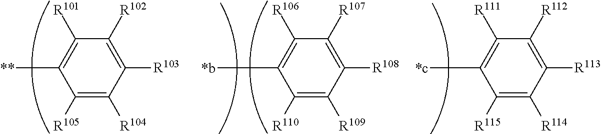

R¹¹¹ to R¹¹⁵ each independently represent a hydrogen atom, a substituted or unsubstituted alkyl group having 1 to 6 carbon atoms, or a substituted or unsubstituted heteroaryl group (heterocyclic group) having 5 to 13 ring atoms.

Adjacent two selected from R¹¹¹ to R¹¹⁵, i.e., at least one adjacent pair selected from R¹¹¹ and R¹¹², R¹¹² and R¹¹³, R¹¹³ and R¹¹⁴, and R¹¹⁴ and R¹¹⁵, may be bonded to each other to form one or multiple unsubstituted benzene rings, or may not be bonded to each other, and therefore may not form a ring.

The details of the substituted or unsubstituted alkyl group having 1 to 6 carbon atoms have been described for R^{a} and R^{b} except that the number of carbon atoms thereof is 1 to 6.

In still another embodiment of the present invention, m represents 2, and n represents 1. In this case, the formula (1a) is represented by the following formula.

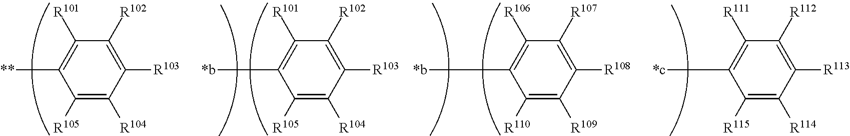

The substituted or unsubstituted heteroaryl group having 5 to 13 ring atoms is, for example,
a pyrrolyl group, a furyl group, a thienyl group, a pyridyl group, a pyridazinyl group, a pyrimidinyl group, a pyrazinyl group, a triazinyl group, an imidazolyl group, an oxazolyl group, a thiazolyl group, an indolyl group, a quinolizinyl group, a quinolyl group, a benzofuranyl group, a benzothiophenyl group (i.e., a benzothienyl group, hereinafter the same), dibenzofuranyl group, a dibenzothiophenyl group (i.e., a dibenzothienyl group, hereinafter the same), or a carbazolyl group (encompassing a 9-carboazolyl group and a 1-, 2-, 3-, or 4-carbazoly group, hereinafter the same),
preferably a benzofuranyl group, a dibenzofuranyl group, a dibenzothiophenyl group, a benzothiophenyl group, or a carbazolyl group, and
more preferably a dibenzofuranyl group, a dibenzothiophenyl group, or a carbazolyl group.

All R¹¹¹ to R¹¹⁵ may represent hydrogen atoms.

The formula (1a) encompasses groups represented by the following formulae (1a-1) to (1a-7), and the formula (1a-3) is preferred. In the following formulae, R is omitted for simplicity.

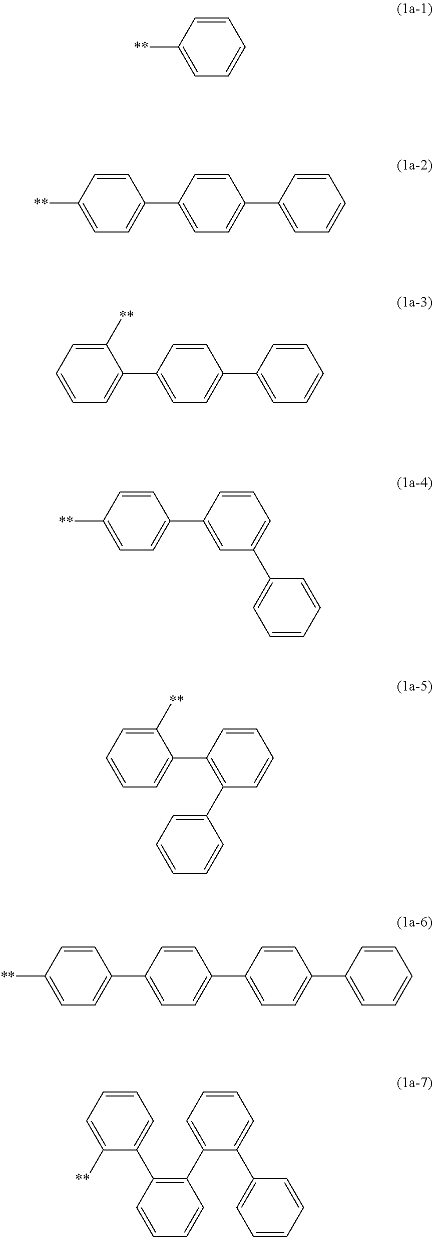

In one embodiment of the present invention, it is preferred that one selected from R¹⁰¹ to R¹⁰⁵ represents a single bond bonded to *b, or one selected from R¹⁰⁶ to R¹¹⁰ represents a single bond bonded to *c.

The formula (1b) is represented by the following formula.

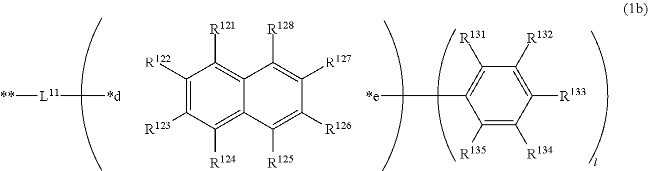

In the formula (1b),
** represents a bonding site to the center nitrogen atom N*.

L¹¹ represents a single bond, an unsubstituted arylene group having 6 to 12 ring carbon atoms, or an unsubstituted divalent heterocyclic group having 5 to 13 ring atoms.

The unsubstituted arylene group having 6 to 12 ring carbon atoms represented by L¹¹ is a divalent group having 6 to 12 ring carbon atoms that is obtained by removing one hydrogen atom from the unsubstituted aryl group described for R^{c} and R^{d}, and is preferably an o-, m-, or p-phenylene group, a 4,4′-, 4,3′-, or 4,2′-biphenyldiyl group, or a 1,4- or 2,6-naphthylene group, and more preferably an o-, m-, or p-phenylene group.

The unsubstituted divalent heterocyclic group having 5 to 13 ring atoms represented by Ln is a divalent group that is obtained by removing one hydrogen atom from the unsubstituted heteroaryl group described for R¹¹¹ to R¹¹⁵, and is preferably a benzofuranylene group, a benzothiophenylene group, a dibenzofuranylene group, a dibenzothiophenylene group, or a carbazolylene group, and
more preferably a dibenzofuranylene group, a dibenzothiophenylene group, or a carbazolylene group.

L¹¹ preferably represents a single bond or an unsubstituted arylene group having 6 to 12 ring carbon atoms, and more preferably a single bond.

R¹²¹ to R¹²⁸ each independently represent a hydrogen atom, a substituted or unsubstituted alkyl group having 1 to 6 carbon atoms, or a substituted or unsubstituted aryl group having 6 to 12 ring carbon atoms.

One selected from R¹²¹ to R¹²⁸ represents a single bond bonded to *d, the other one selected from R¹²¹ to R¹²⁸ represents a single bond bonded to *e.

Adjacent two selected from R¹²¹ to R¹²⁸ that are not the single bond bonded to *d and a single bond bonded to *e are not bonded to each other, and therefore do not form a ring.

The details of the unsubstituted alkyl group having 1 to 6 carbon atoms have been described for R^{a} and R^{b} except that the number of carbon atoms thereof is 1 to 6.

The details of the unsubstituted aryl group having 6 to 12 ring carbon atoms have been described for R^{c} and R^{d} except that the number of ring carbon atoms thereof is 6 to 12.

In one embodiment of the present invention, R¹²¹ preferably represents a single bond bonded to *d, and in another embodiment thereof, R¹²² preferably represents a single bond bonded to *d.

All R¹²¹ to R¹²⁸ that are not the single bond are not bonded may represent hydrogen atoms.

R¹³¹ to R¹³⁵ each independently represent a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

Adjacent two selected from R¹³¹ to R¹³⁵, i.e., at least one adjacent pair selected from R¹³¹ and R¹³², R¹³² and R¹³³, R¹³³ and R¹³⁴, and R¹³⁴ and R¹³⁵, may be bonded to each other to form one or multiple unsubstituted benzene rings, or may not be bonded to each other, and therefore may not form a ring.

The details of the unsubstituted alkyl group having 1 to 6 carbon atoms have been described for R^{a} and R^{b} except that the number of carbon atoms thereof is 1 to 6.

The details of the unsubstituted aryl group having 6 to 12 ring carbon atoms have been described for R^{c} and R^{d} except that the number of ring carbon atoms thereof is 6 to 12.

The details of the unsubstituted heteroaryl group having 5 to 13 ring atoms have been described for R¹¹¹ to R¹¹⁵.

All R¹³¹ to R¹³⁵ may represent hydrogen atoms.

l represents 0 or 1.

In one embodiment of the present invention, l represents 0, and in another embodiment of the present invention l represents 1.

The formula (1c) is represented by the following formula.

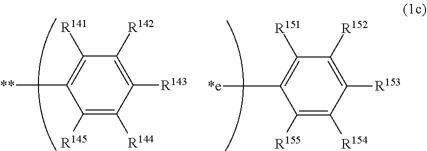

In the formula (1c),
** represents a bonding site to the center nitrogen atom N*.

R¹⁴¹ to R¹⁴⁵ each independently represent a hydrogen atom or a substituted or unsubstituted alkyl group having 1 to 6 carbon atoms.

One selected from R¹⁴¹, R¹⁴², R¹⁴⁴, and R¹⁴⁵ represents a single bond bonded to *e.

Adjacent two selected from R¹⁴³, and R¹⁴¹, R¹⁴², R¹⁴⁴, and R¹⁴⁵ that are not the single bond bonded to *e are not bonded to each other, and therefore do not form a ring.

The details of the unsubstituted alkyl group having 1 to 6 carbon atoms have been described for R^{a} and R^{b} except that the number of carbon atoms thereof is 1 to 6.

In one embodiment of the present invention, one selected from R¹⁴¹ and R¹⁴⁵ preferably represents a single bond bonded to *e.

In another embodiment of the present invention, one selected from R¹⁴² and R¹⁴⁴ preferably represents a single bond bonded to *e.

All R¹⁴¹, R¹⁴², R¹⁴⁴, and R¹⁴⁵ that are not the single bond may represent hydrogen atoms.

R¹⁵¹ to R¹⁵⁵ each independently represent a hydrogen atom or a substituted or unsubstituted alkyl group having 1 to 6 carbon atoms.

Adjacent two selected from R¹⁵¹ to R¹⁵⁵ are not bonded to each other, and therefore do not form a ring.

The details of the unsubstituted alkyl group having 1 to 6 carbon atoms have been described for R^{a} and R^{b} except that the number of carbon atoms thereof is 1 to 6.

All R¹⁵¹ to R¹⁵⁵ may represent hydrogen atoms.

The formula (1d) is represented by the following formula.

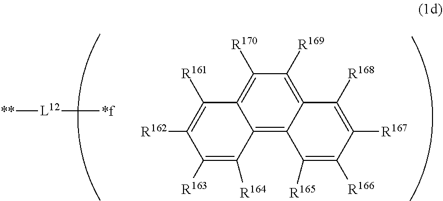

In the formula (1d),
** represents a bonding site to the center nitrogen atom N*.

L¹² represents a single bond, an unsubstituted arylene group having 6 to 12 ring carbon atoms, or an unsubstituted divalent heterocyclic group having 5 to 13 ring atoms.

The details of the unsubstituted arylene group having 6 to 12 ring carbon atoms have been described for L¹¹.

The details of the unsubstituted divalent heterocyclic group having 5 to 13 ring atoms have been described for L¹¹.

L¹² preferably represents a single bond or an unsubstituted arylene group having 6 to 12 ring carbon atoms, and more preferably represents a single bond.

R¹⁶¹ to R¹⁷⁰ each independently represent a hydrogen atom, a substituted or unsubstituted alkyl group having 1 to 6 carbon atoms, or a substituted or unsubstituted aryl group having 6 to 12 ring carbon atoms.

In the case where L¹² represents a single bond, one selected from R¹⁶¹ to R¹⁶⁸ represents a single bond bonded to *f, and in the case where L¹² represents an unsubstituted arylene group having 6 to 12 ring carbon atoms or an unsubstituted divalent heterocyclic group having 5 to 13 ring atoms, one selected from R¹⁶¹ to R¹⁷⁰ represents a single bond bonded to *f, and

Adjacent two selected from R¹⁶¹ to R¹⁷⁰ that are not the single bond are not bonded to each other, and therefore do not form a ring.

The details of the unsubstituted alkyl group having 1 to 6 carbon atoms have been described for R^{a} and R^{b} except that the number of carbon atoms thereof is 1 to 6.

The details of the unsubstituted aryl group having 6 to 12 ring carbon atoms have been described for R^{c} and R^{d} except that the number of ring carbon atoms thereof is 6 to 12.

All R¹⁶¹ to R¹⁷⁰ that are not the single bond may represent hydrogen atoms.

The formula (1e) is represented by the following formula.

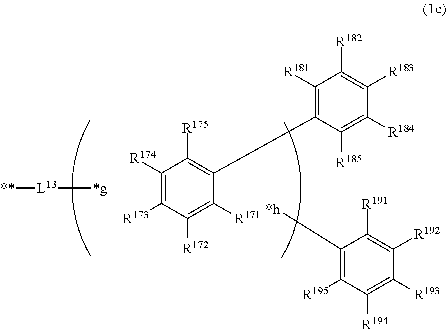

In the formula (1e),
** represents a bonding site to the center nitrogen atom N*,

L¹³ represents a single bond, an unsubstituted arylene group having 6 to 12 ring carbon atoms, or an unsubstituted divalent heterocyclic group having 5 to 13 ring atoms.

The details of the unsubstituted arylene group having 6 to 12 ring carbon atoms have been described for L¹¹.

The details of the unsubstituted divalent heterocyclic group having 5 to 13 ring atoms have been described for L¹¹.

L¹³ preferably represents a single bond or an unsubstituted arylene group having 6 to 12 ring carbon atoms, and more preferably represents a single bond.

R¹⁷¹ to R¹⁷⁵ each independently represent a hydrogen atom, an unsubstituted alkyl group having 1 to 6 carbon atoms, or an unsubstituted phenyl group.

One selected from R¹⁷¹ to R¹⁷⁵ represents a single bond bonded to *g, and the other one selected from R¹⁷¹ and R¹⁷² represents a single bond bonded to *h.

Adjacent two selected from R¹⁷¹ to R¹⁷⁵ that are not the single bond bonded to *g and the single bond bonded to *h are not bonded to each other, and therefore do not form a ring.

The details of the unsubstituted alkyl group having 1 to 6 carbon atoms have been described for R^{a} and R^{b} except that the number of carbon atoms thereof is 1 to 6.

One selected from R¹⁷¹ to R¹⁷⁵ represents a single bond bonded to *g, and the other one selected from R¹⁷¹ and R¹⁷² represents a single bond bonded to *h.

All R¹⁷¹ to R¹⁷⁶ that are not the single bond may represent hydrogen atoms.

R¹⁸¹ to R¹⁸⁵ and R¹⁹¹ to R¹⁹⁵ each independently represent a hydrogen atom or an unsubstituted alkyl group having 1 to 6 carbon atoms.

Adjacent two selected from R¹⁸¹ to R¹⁸⁵ and R¹⁹¹ to R¹⁹⁵, i.e., at least one adjacent pair selected from R¹⁸¹ and R¹⁸², R¹⁸² and R¹⁸³, R¹⁸³ and R¹⁸⁴, R¹⁸⁴ and R¹⁸⁵, R¹⁹¹ and R¹⁹², R¹⁹² and R¹⁹³, R¹⁹³ and R¹⁹⁴, and R¹⁹⁴ and R¹⁹⁵ may be bonded to each other to form one or multiple unsubstituted benzene rings, or may not be bonded to each other, and therefore may not form a ring.

The details of the unsubstituted alkyl group having 1 to 6 carbon atoms have been described for R^{a} and R^{b} except that the number of carbon atoms thereof is 1 to 6.

All R¹⁸¹ to R¹⁸⁵ and R¹⁹¹ to R¹⁹⁵ may represent hydrogen atoms.

The formula (1e) encompasses groups represented by the following formulae (1e-1) to (1e-5), and the formula (1e-4) is preferred.

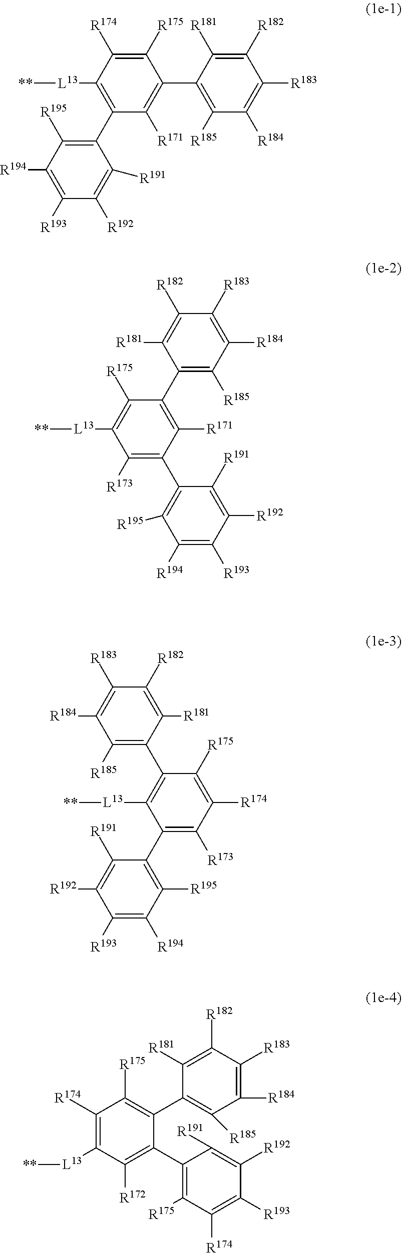

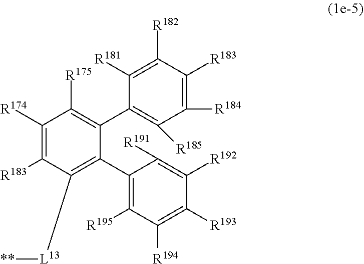

The formula (1f) is represented by the following formula.

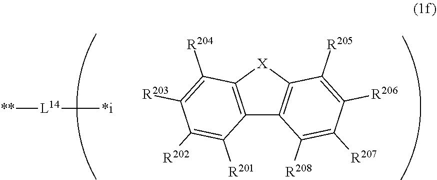

In the formula (1f),
**represents a bonding site to the center nitrogen atom N*.

L¹⁴ represents a single bond or an unsubstituted phenylene group.

In one embodiment of the present invention, L¹⁴ preferably represents a single bond.

In another embodiment of the present invention, L¹⁴ preferably represents a phenylene group.

X represents an oxygen atom, a sulfur atom, or CR^{A}R^{B}.

X preferably represents CR^{A}R^{B}.

R^{A} and R^{B} each independently represent a hydrogen atom, a substituted or unsubstituted alkyl group having 1 to 30, preferably 1 to 18, and more preferably 1 to 6, carbon atoms, or a substituted or unsubstituted aryl group having 6 to 30, preferably 6 to 25, and more preferably 6 to 12, ring carbon atoms.

R^{A} and R^{B} may be bonded to each other to form a substituted or unsubstituted ring, or may not be bonded to each other, and therefore may not form a ring.

The details of the substituted or unsubstituted alkyl group having 1 to 30 carbon atoms represented by R^{A} and R^{B} have been described for R^{a} and R^{b}.

The details of the substituted or unsubstituted aryl group having 6 to 30 ring carbon atoms represented by R^{A} and R^{B} have been described for R^{c} and R^{d}.

In one embodiment of the present invention, R^{A} and R^{B} each preferably represent a substituted or unsubstituted alkyl group having 1 to 30, more preferably represent a methyl group, an ethyl group, a n-propyl group, an isopropyl group, a n-butyl group, an isobutyl group, a s-butyl group, or a t-butyl group, and further preferably a methyl group or a t-butyl group.

The details of the substituted or unsubstituted ring formed by bonding R^{A} and R^{B} to each other have been described for R^{a} and R^{b}.

R²⁰¹ to R²⁰⁸ each independently represent a hydrogen atom, a substituted or unsubstituted alkyl group having 1 to 6 carbon atoms, or a substituted or unsubstituted aryl group having 6 to 12 ring carbon atoms.

In the case where L¹⁴ represents a phenylene group, and X represents an oxygen atom, one selected from R²⁰¹ to R²⁰³, R²⁰⁶ to R²⁰⁸, R^{A}, and R^{B} represents a single bond bonded to *i, and
in the case where L¹⁴ represents a single bond, and X represents an oxygen atom, a sulfur atom, or CR^{A}R^{B}, and in the case where L¹⁴ represents a phenylene group, and X represents a sulfur atom or CR^{A}R^{B}, one selected from R²⁰¹ to R²⁰⁸, R^{A}, and R^{B} represents a single bond bonded to *i.

In the case where X represents CR^{A}R^{B}, adjacent two selected from R²⁰¹ to R²⁰⁸ that are not the single bond are not bonded to each other, and therefore do not form a ring.

In the case where X represents an oxygen atom or a sulfur atom, adjacent two selected from R²⁰¹ to R²⁰⁸, i.e., at least one adjacent pair selected from R²⁰¹ and R²⁰², R²⁰² and R²⁰³, R²⁰³ and R²⁰⁴, R²⁰⁵ and R²⁰⁶, R²⁰⁶ and R²⁰⁷, and R²⁰⁷ and R²⁰⁸, that are not the single bond may be bonded to each other to form one or multiple unsubstituted benzene rings, or may not be bonded to each other, and therefore may not form a ring.

The details of the unsubstituted alkyl group having 1 to 6 carbon atoms have been described for R^{a} and R^{b} except that the number of carbon atoms thereof is 1 to 6.

The details of the unsubstituted aryl group having 6 to 12 ring carbon atoms have been described for R^{c} and R^{d} except that the number of ring carbon atoms thereof is 6 to 12.

In the case where X represents CR^{A}R^{B}, it is particularly preferred that both R^{A} and R^{B} represent unsubstituted phenyl groups or unsubstituted methyl groups, or one thereof represents an unsubstituted phenyl group, and the other thereof represents an unsubstituted methyl group.

All R²⁰¹ to R²⁰⁸ that are not the single bond may represent hydrogen atoms.

The formula (1f) is represented by any of the following formulae.

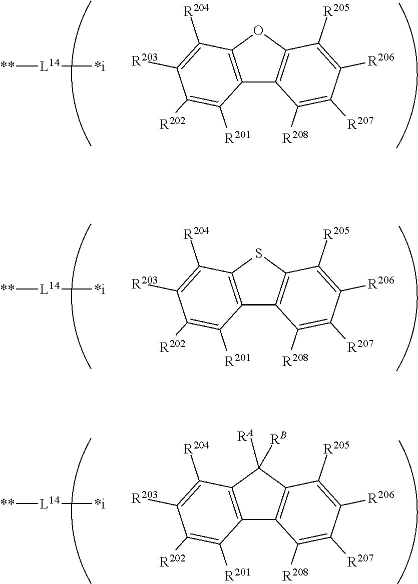

In one embodiment of the present invention, Ar¹ preferably represents a group represented by the formula (1a) or (1f), and more preferably represents a group represented by the formula (1a).

As described above, the “hydrogen atom” referred in the description herein encompasses a protium atom, a deuterium atom, and tritium atom. Accordingly, the inventive compound may contain a naturally derived deuterium atom.

A deuterium atom may be intentionally introduced into the inventive compound by using a deuterated compound as a part or the whole of the raw material. Accordingly, in one embodiment of the present invention, the inventive compound contains at least one deuterium atom. Accordingly, the inventive compound may be a compound represented by the formula (1), in which at least one hydrogen atom contained in the compound is a deuterium atom.

At least one hydrogen atom selected from the following hydrogen atoms may be a deuterium atom. In the following, the term “substituted or unsubstituted”, the number of carbon atoms, and the number of atoms are omitted for convenience:
in the case where L¹ and L² in the formula (1) represent a phenylene group, the hydrogen atoms of the phenylene group;
the hydrogen atoms represented by R¹ to R⁷ and R¹¹ to R¹⁷ in the formula (1);
in the case where R¹ to R⁷ and R¹¹ to R¹⁷ in the formula (1) represent an alkyl group, the hydrogen atoms of the alkyl group:
the hydrogen atoms represented by R^{a} and R^{b} in the formula (1);
in the case where R^{a} and R^{b} in the formula (1) represent an alkyl group, the hydrogen atoms of the alkyl group;
in the case where R^{a} and R^{b} in the formula (1) are bonded to each other to form a substituted or unsubstituted ring, the hydrogen atoms of the ring;
the hydrogen atoms represented by R^{c} and R^{d} in the formula (1);
in the case where R^{c} and R^{d} in the formula (1) represents an alkyl group or an aryl group, the hydrogen atoms of these groups;
in the case where R^{c} and R^{d} in the formula (1) are bonded to each other to form a substituted or unsubstituted ring, the hydrogen atoms of the ring;
the hydrogen atoms represented by R²¹ to R²⁵ that are not the single bond bonded to *a in the formula (1);
the hydrogen atoms represented by R¹⁰¹ to R¹⁰⁵ and R¹⁰⁶ to R¹¹⁰ that are not the single bond bonded to *b and the single bond bonded to *c in the formula (1a);
in the case where R¹⁰¹ to R¹⁰⁵ and R¹⁰⁶ to R¹¹⁰ in the formula (1a) represent an alkyl group, the hydrogen atoms of the alkyl group;
the hydrogen atoms represented by R¹¹¹ to R¹¹⁵ in the formula (1a);
in the case where R¹¹¹ to R¹¹⁵ in the formula (1a) represent an alkyl group or a heteroaryl group, the hydrogen atoms of these groups;
in the case where adjacent two selected from R¹¹¹ to R¹¹⁵ in the formula (1a) are bonded to each other to form an unsubstituted benzene ring, the hydrogen atoms of the benzene ring;
in the case where L¹¹ in the formula (1b) represents an arylene group or a divalent heterocyclic group, the hydrogen atoms of these groups;
the hydrogen atoms represented by R¹²¹ to R¹²⁸ that are not the single bond bonded to *d and the single bond bonded to *e in the formula (1b);
in the case where R¹²¹ to R¹²⁸ in the formula (1b) represent an alkyl group or an aryl group, the hydrogen atoms of these groups;
the hydrogen atoms represented by R¹³¹ to R¹³⁵ in the formula (1b);
in the case where R¹³¹ to R¹³⁵ in the formula (1b) represent an alkyl group, an aryl group, or a heteroaryl group, the hydrogen atoms of these groups;
in the case where adjacent two selected from R¹⁸¹ to R¹⁸³ in the formula (1b) are bonded to each other to form an unsubstituted benzene ring, the hydrogen atoms of the benzene ring;
the hydrogen atoms represented by R¹⁴¹ to R¹⁴⁵ that are not the single bond bonded to *e in the formula (1c);
in the case where R¹⁴¹ to R¹⁴⁵ in the formula (1c) represent an alkyl group, the hydrogen atoms of the alkyl group;
the hydrogen atoms represented by R¹⁵¹ to R¹⁵⁵ in the formula (1c);
in the case where R¹⁵¹ to R¹⁵⁵ in the formula (1c) represent an alkyl group, the hydrogen atoms of the alkyl group;
in the case where L¹² in the formula (1d) represents an arylene group or a divalent heterocyclic group, the hydrogen atoms of these groups;
the hydrogen atoms represented by R¹⁶¹ to R¹⁷⁰ that are not the single bond bonded to *f in the formula (1d);
in the case where R¹⁶¹ to R¹⁷⁰ in the formula (1d) represent an alkyl group or an aryl group, the hydrogen atoms of these groups;
in the case where L¹³ in the formula (1e) represents an arylene group or a divalent heterocyclic group, the hydrogen atoms of these groups;
the hydrogen atoms represented by R¹⁷¹ to R¹⁷⁵ that are not the single bond bonded to *g and the single bond bonded to *h in the formula (1e);
in the case where R¹⁷¹ to R¹⁷⁵ in the formula (1e) represent an alkyl group or a phenyl group, the hydrogen atoms of these groups;
the hydrogen atoms represented by R¹⁸¹ to R¹⁸⁵ and R¹⁹¹ to R¹⁹⁵ in the formula (1e);
in the case where R¹⁸¹ to R¹⁸⁵ and R¹⁹¹ to R¹⁹⁵ in the formula (1e) represent an alkyl group, the hydrogen atoms of the alkyl group;
in the case where adjacent two selected from R¹⁸¹ to R¹⁸⁵ and R¹⁹¹ to R¹⁹⁵ in the formula (1e) are bonded to each other to form an unsubstituted benzene ring, the hydrogen atoms of the benzene ring;
in the case where L¹⁴ in the formula (1f) represents an arylene group or a phenylene group, the hydrogen atoms of these groups;
the hydrogen atoms represented by R^{A} and R^{B} in the formula (1f);
in the case where R^{A} and R^{B} in the formula (1f) represent an alkyl group or an aryl group, the hydrogen atoms of these groups;
in the case where R^{A} and R^{B} in the formula (1f) are bonded to each other to form a substituted or unsubstituted ring, the hydrogen atoms of the ring;
the hydrogen atoms represented by R²⁰¹ to R²⁰⁸ that are not the single bond bonded to *i in the formula (1f);
in the case where R²⁰¹ to R²⁰⁸ in the formula (1f) represent an alkyl group or an aryl group, the hydrogen atoms of these groups; and
in the case where adjacent two selected from R²⁰¹ to R²⁰⁸ in the formula (1f) are bonded to each other to form an unsubstituted benzene ring, the hydrogen atoms of the benzene ring.

The deuteration rate of the inventive compound depends on the deuteration rates of the raw material compounds used. Even though a raw material having a prescribed deuteration rate is used, a naturally derived protium isotope may be contained at a certain proportion. Accordingly, the embodiments of the deuteration rate of the inventive compound shown below include the proportion in consideration of a slight amount of the naturally derived isotopes, with respect to the proportion obtained by counting the number of deuterium atoms shown in the chemical formula.

The deuteration rate of the inventive compound is preferably 1% or more, more preferably 3% or more, further preferably 5% or more, still further preferably 10% or more, and still more further preferably 50% or more.

The inventive compound may be a mixture of a deuterated compound and a non-deuterated compound, or a mixture of two or more compounds having different deuteration rates from each other. The deuteration rate of the mixture is preferably 1% or more, more preferably 3% or more, further preferably 5% or more, still further preferably 10% or more, and still more further preferably 50% or more, and is less than 100%.

The proportion of the number of deuterium atoms with respect to the number of all the hydrogen atoms in the inventive compound is preferably 1% or more, more preferably 3% or more, further preferably 5% or more, and still further preferably 10% or more, and is 100% or less.

In the case where the “substituted or unsubstituted A group” is a substituted A group, the details of the substituent have been described in the section “Substituent for ‘Substituted or Unsubstituted’” above, and the substituent is preferably an alkyl group having 1 to 6 carbon atoms, an aryl group having 6 to 12 ring carbon atoms, or an aromatic heterocyclic group having 5 to 13 ring atoms, and more preferably an alkyl group having 1 to 6 carbon atoms or an aryl group having 6 to 12 ring carbon atoms. The details of these groups have been described above.

The inventive compound can be readily produced by a person skilled in the art according to Synthesis Examples described later or the known synthesis methods.

Specific examples of the inventive compound of the present invention will be described below, but the inventive compound is not limited to the example compounds below.

In the following specific examples, D represents a deuterium atom.

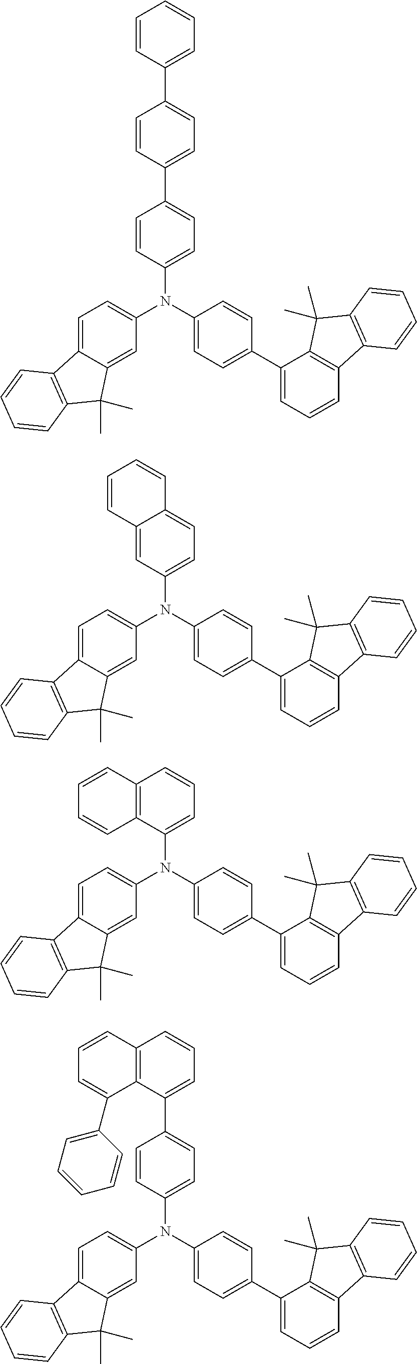

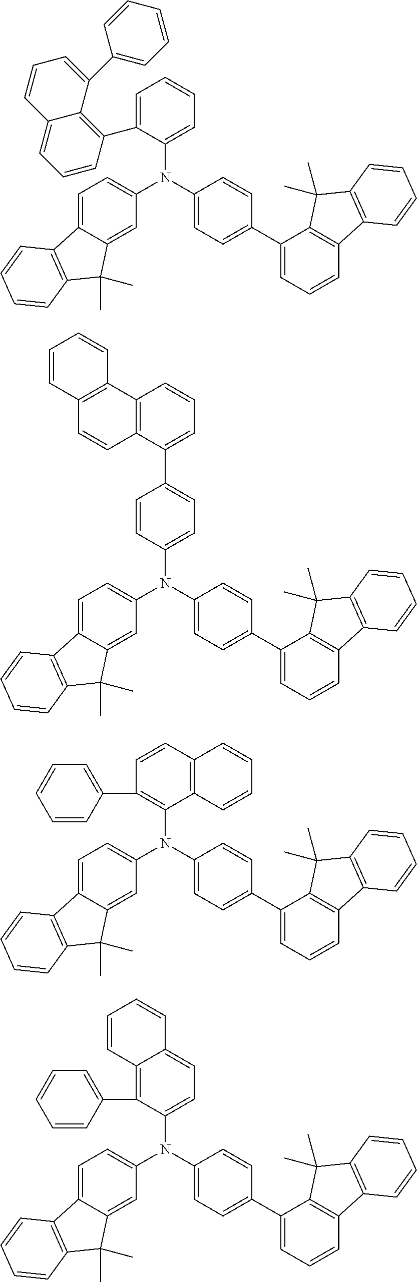

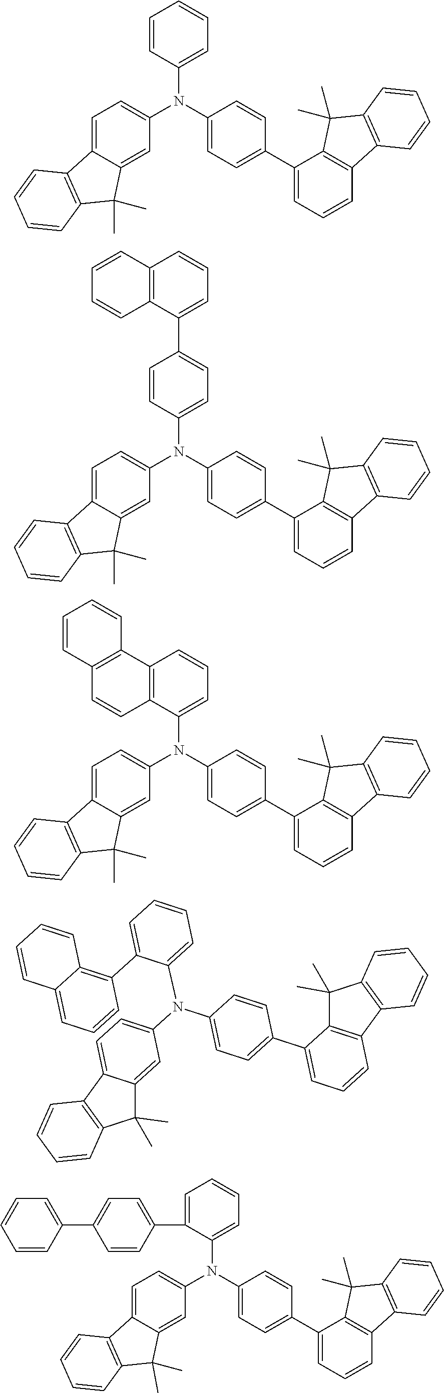

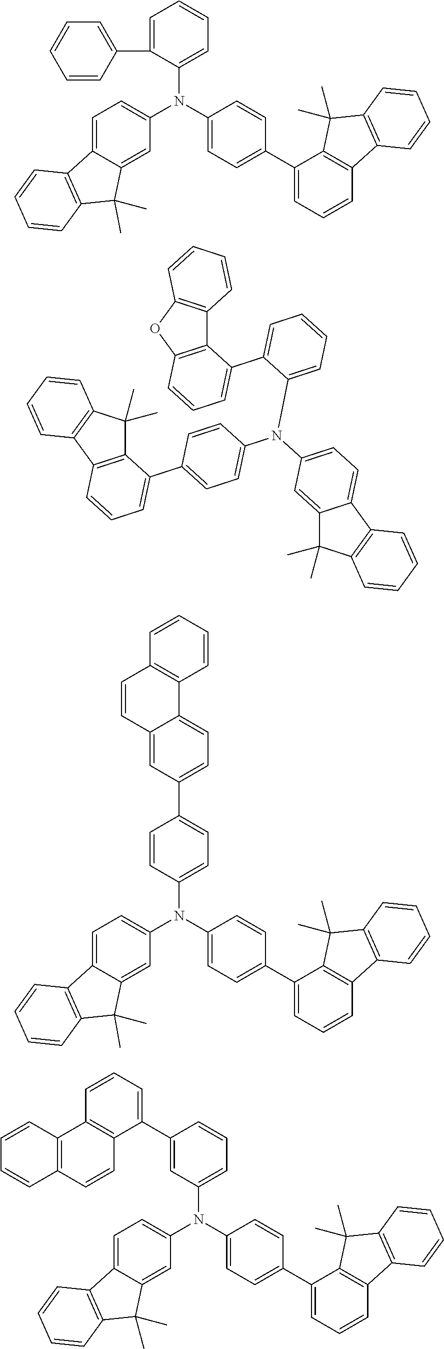

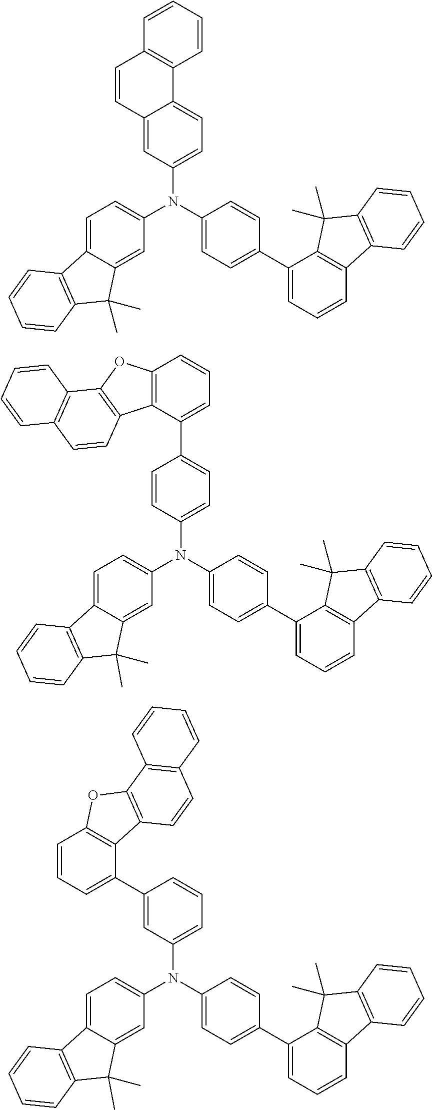

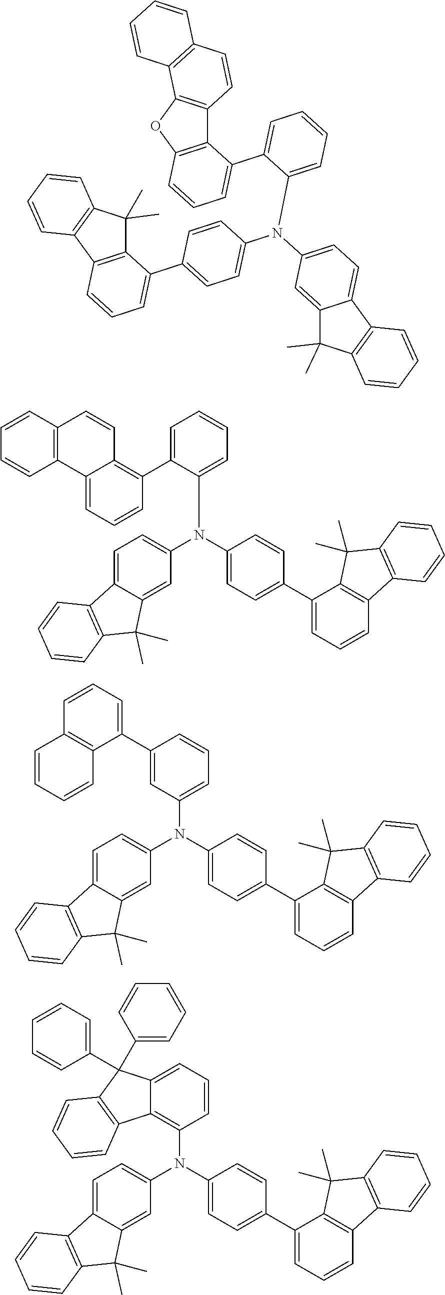

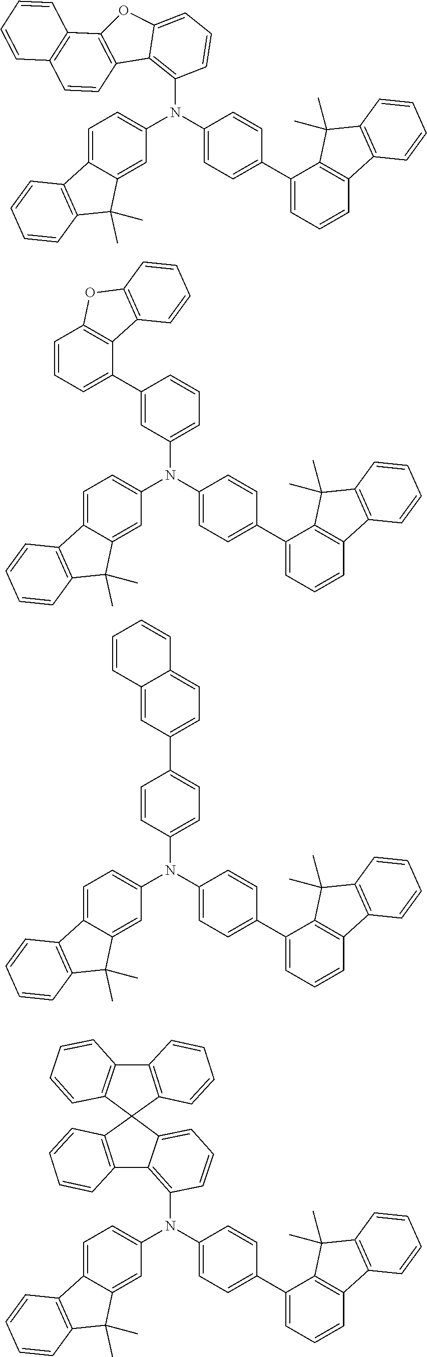

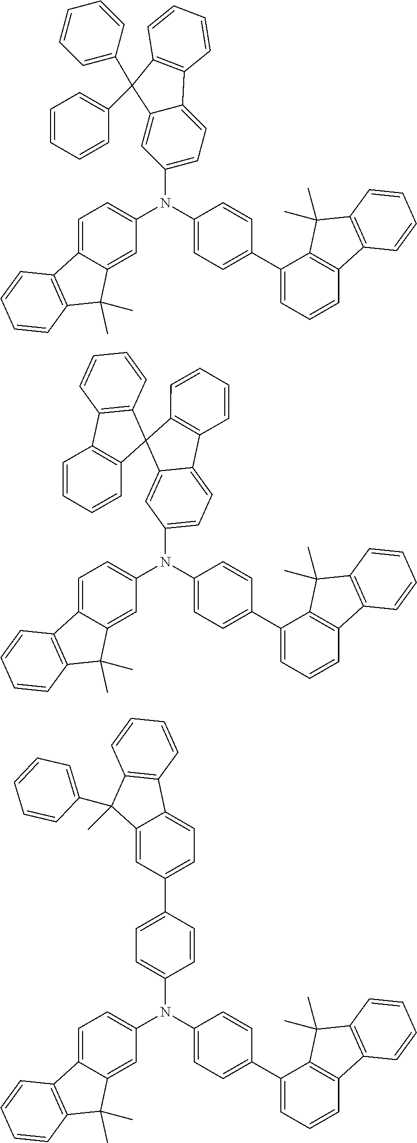

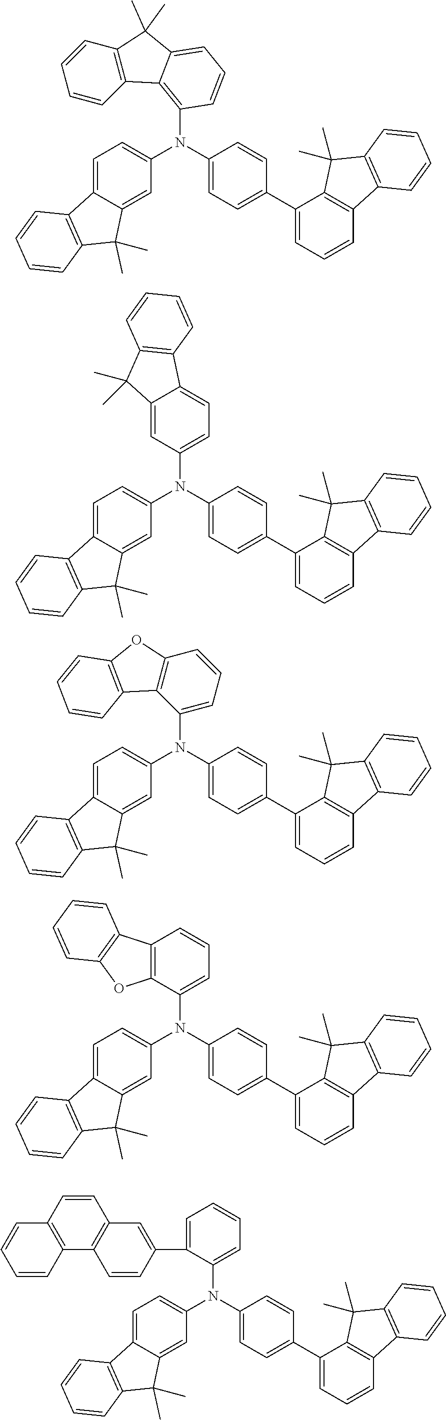

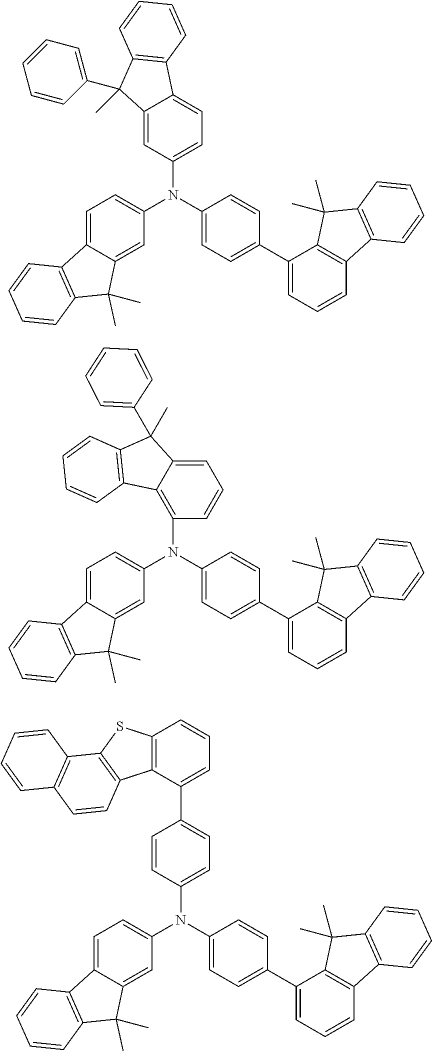

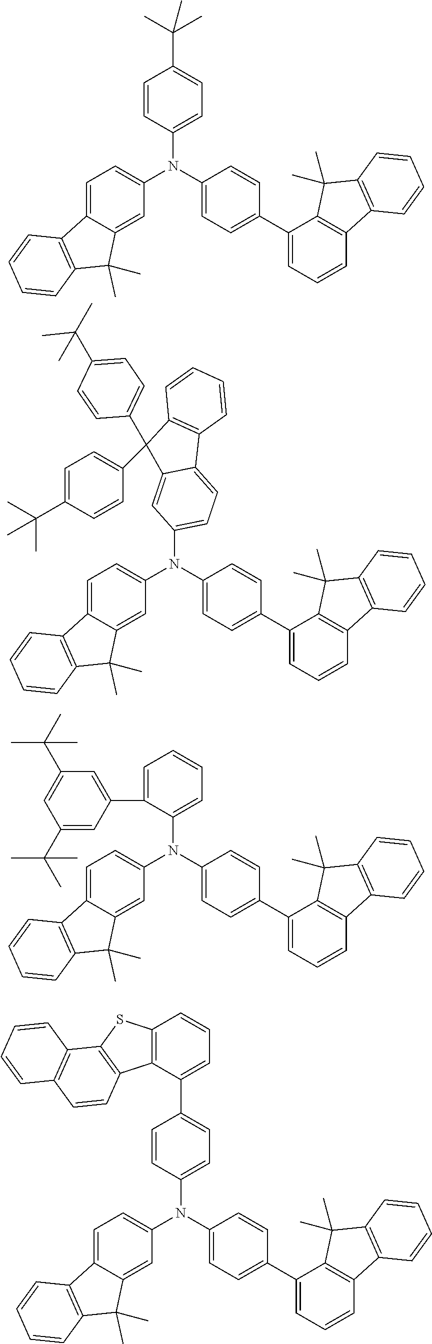

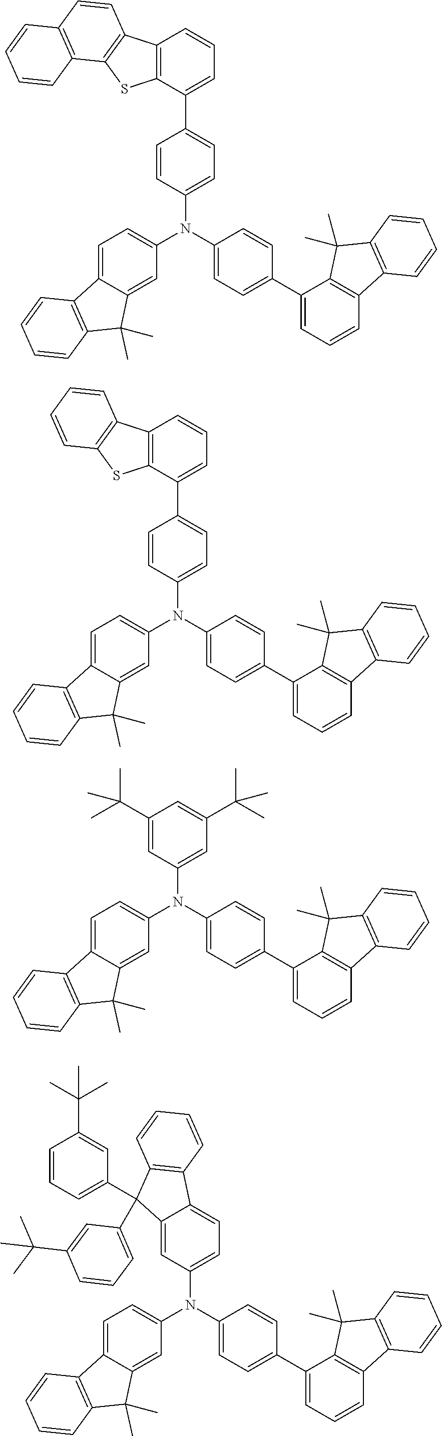

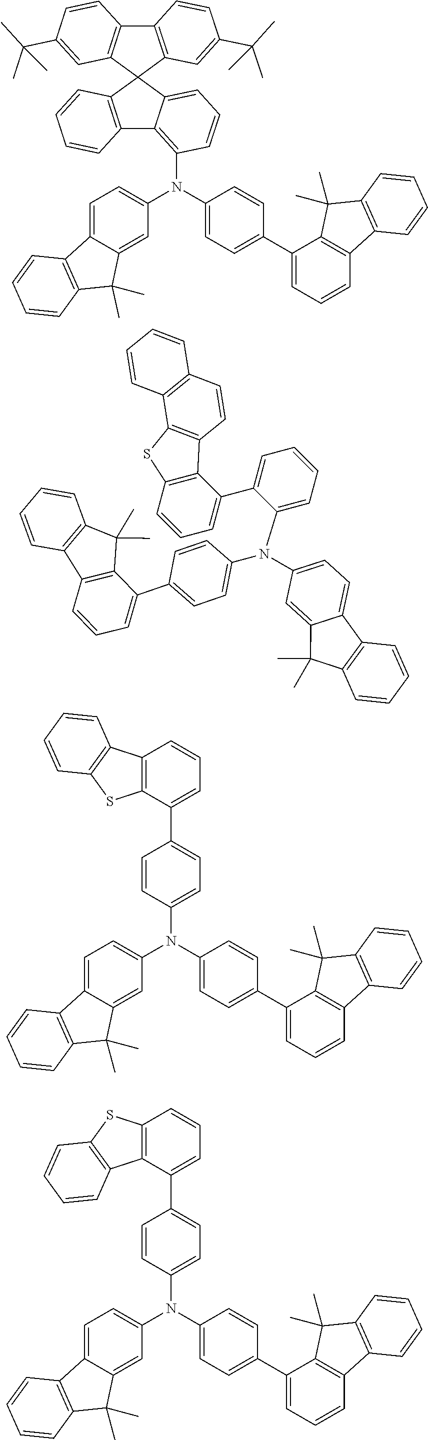

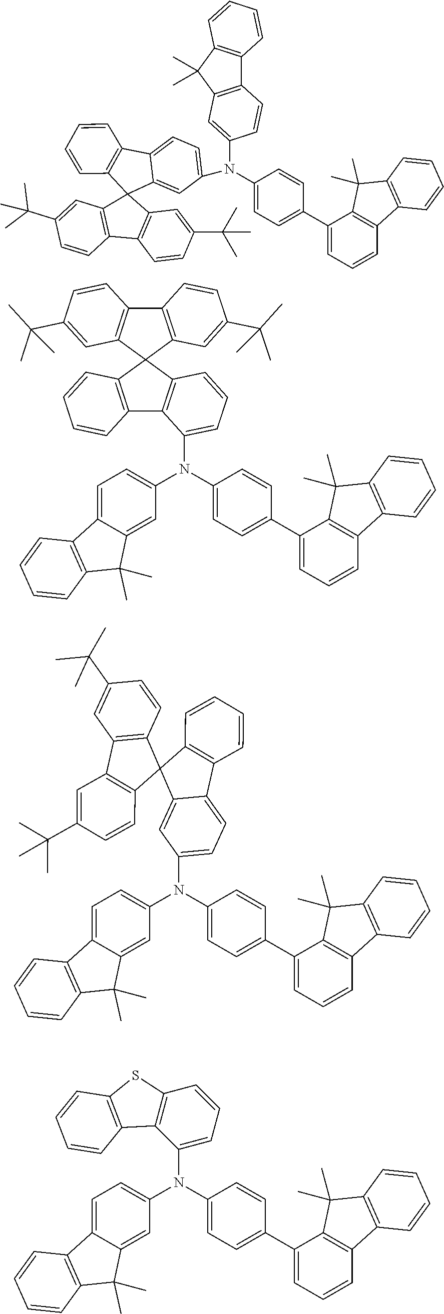

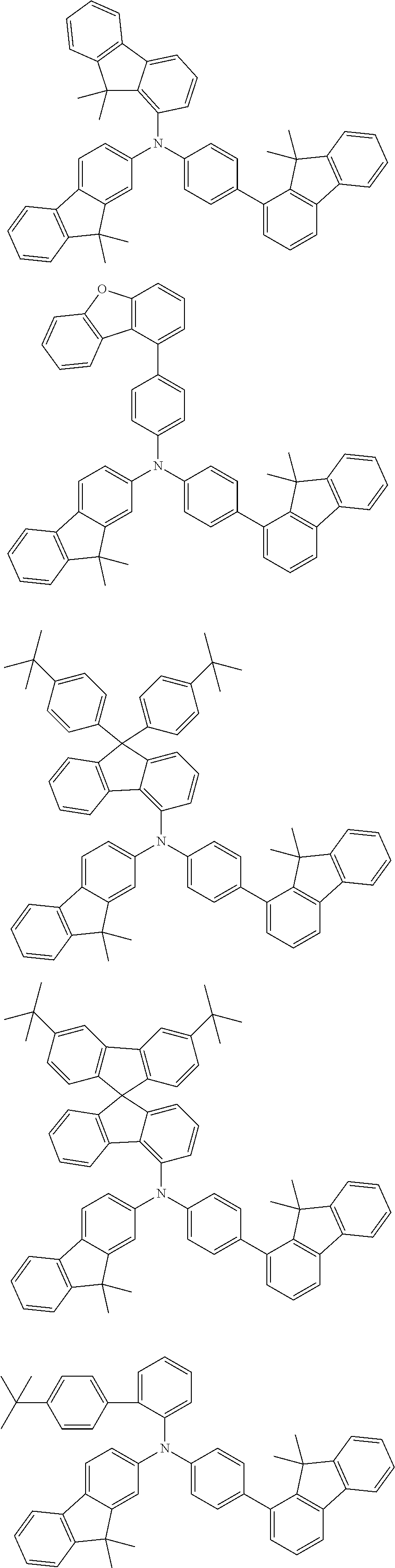

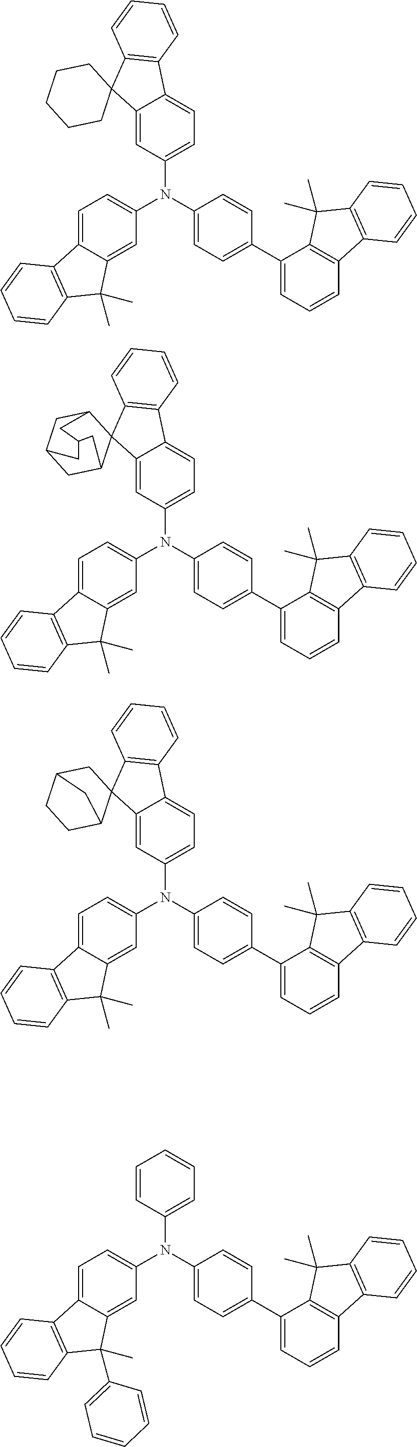

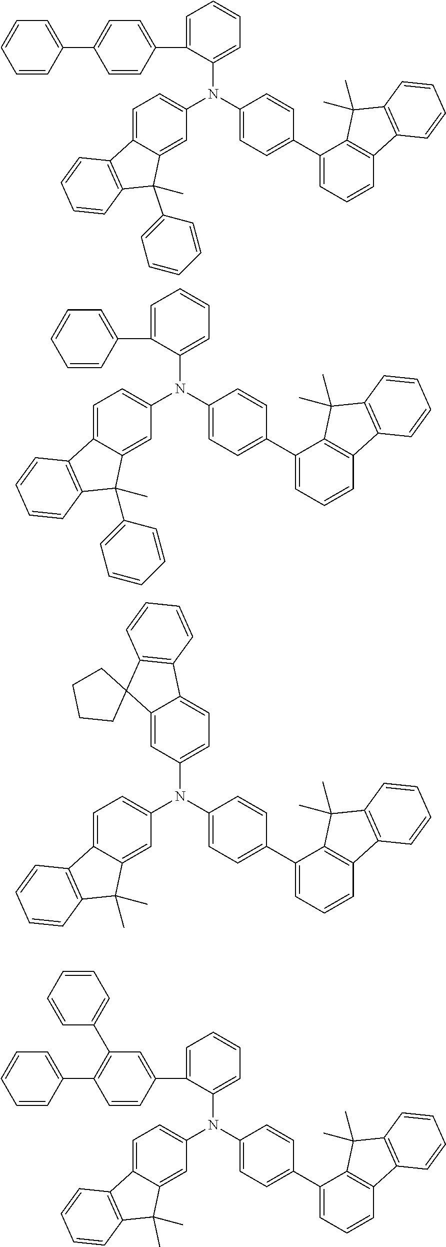

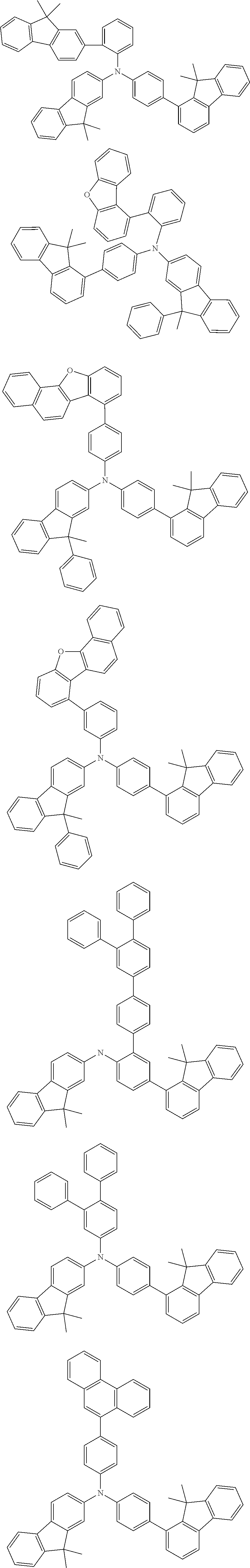

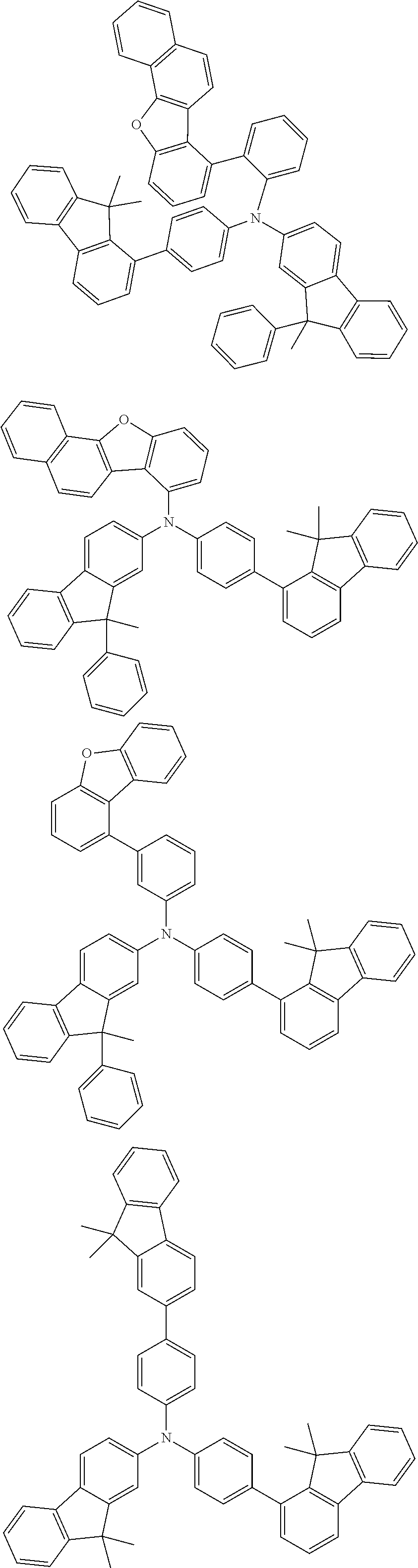

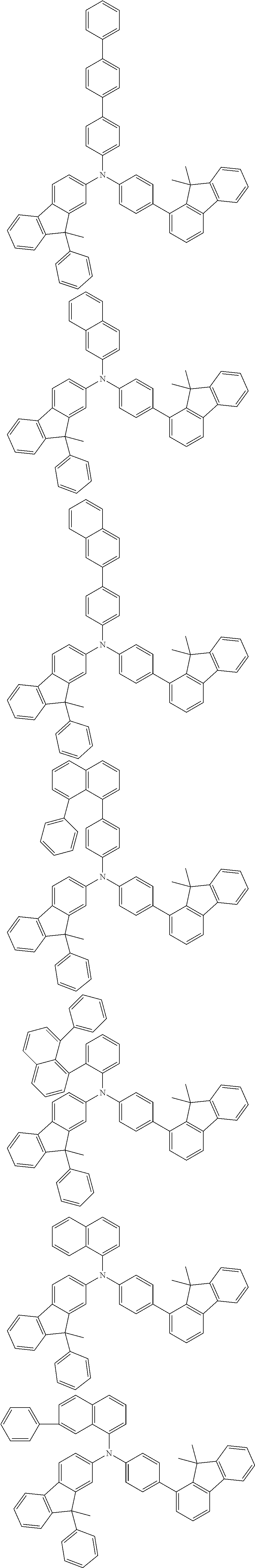

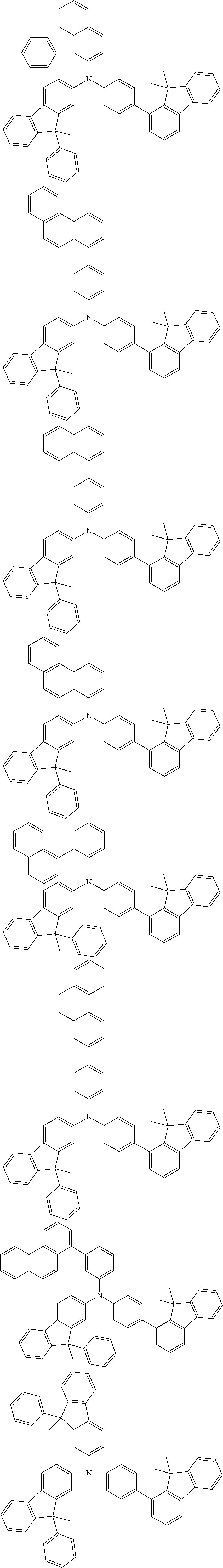

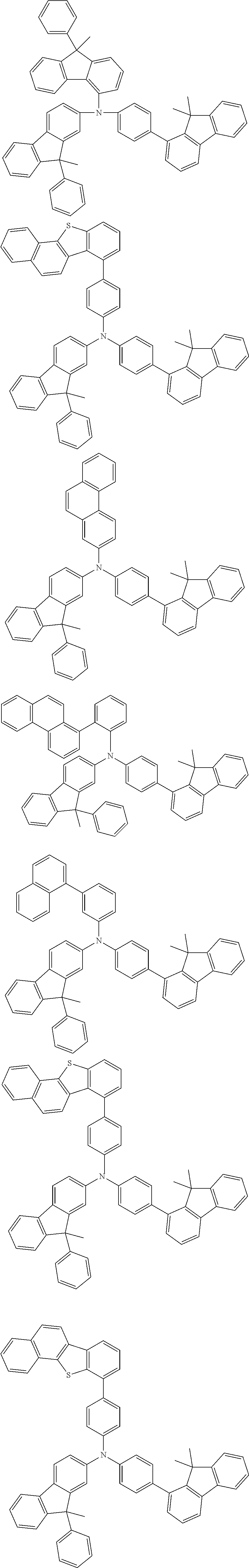

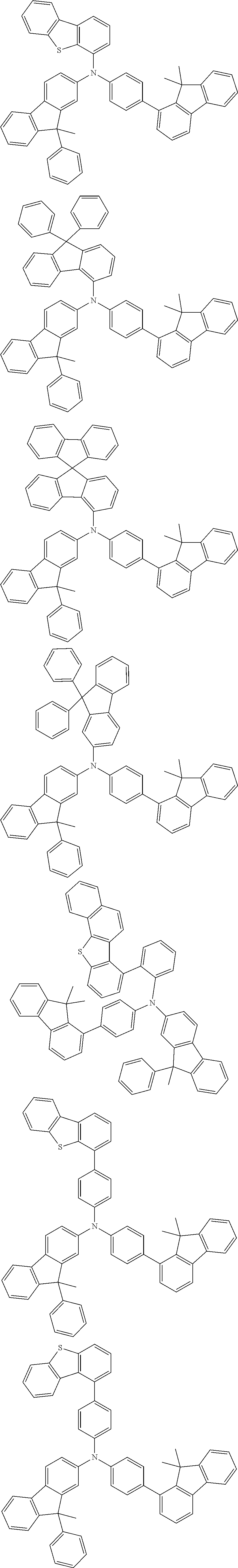

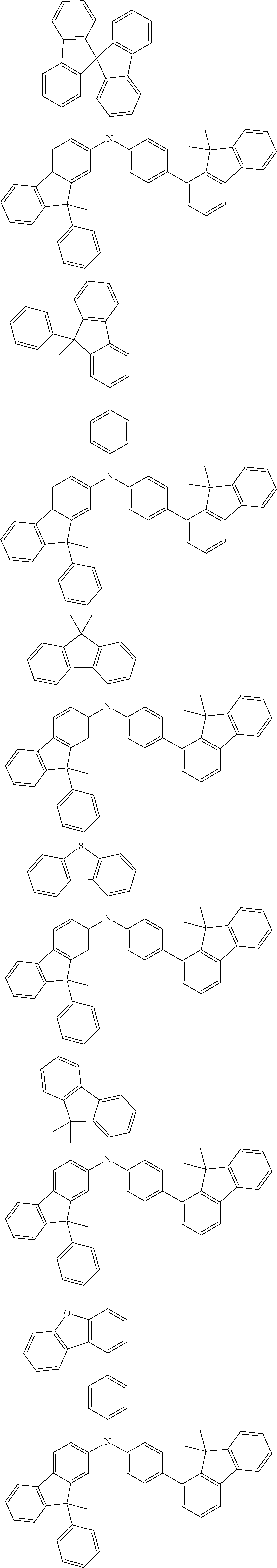

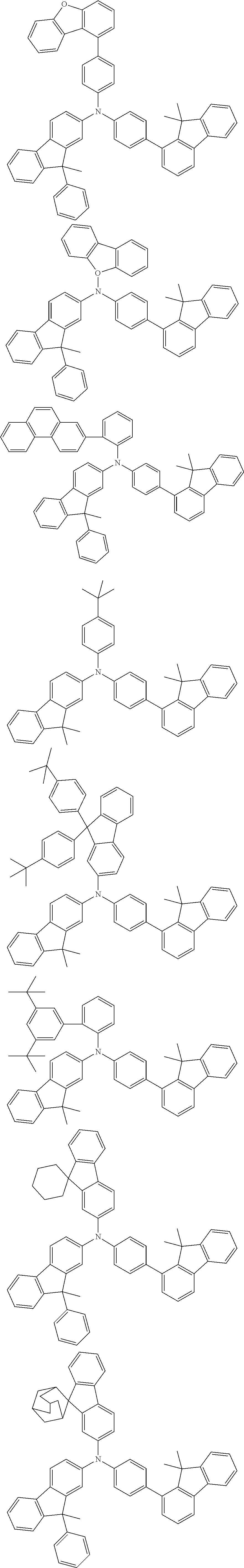

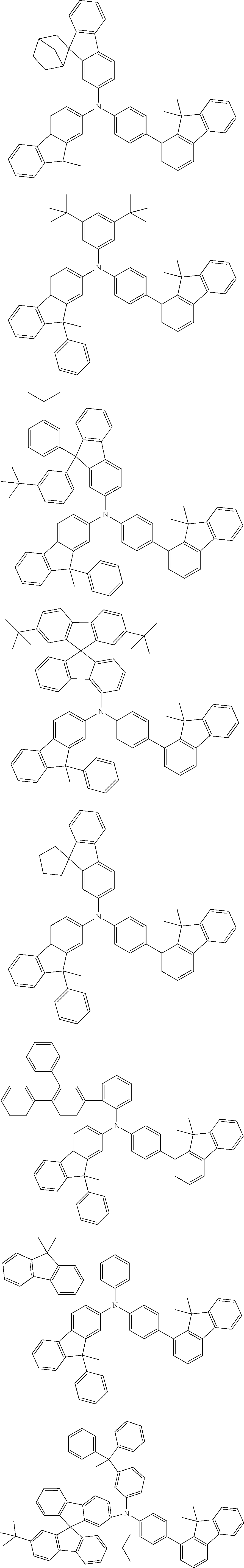

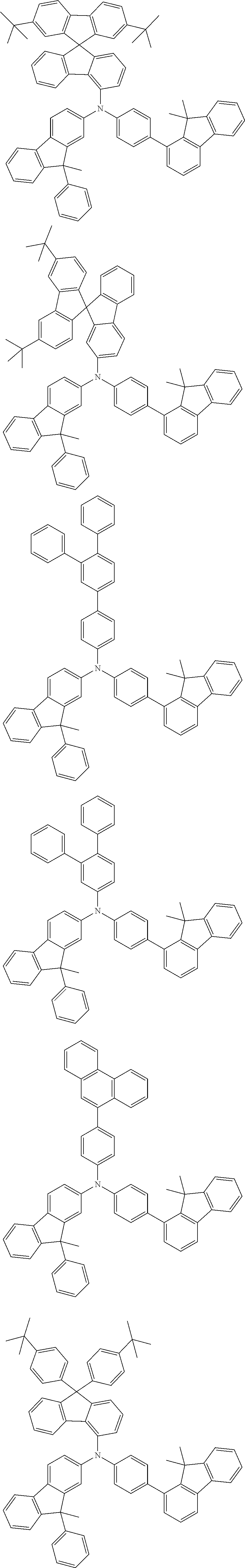

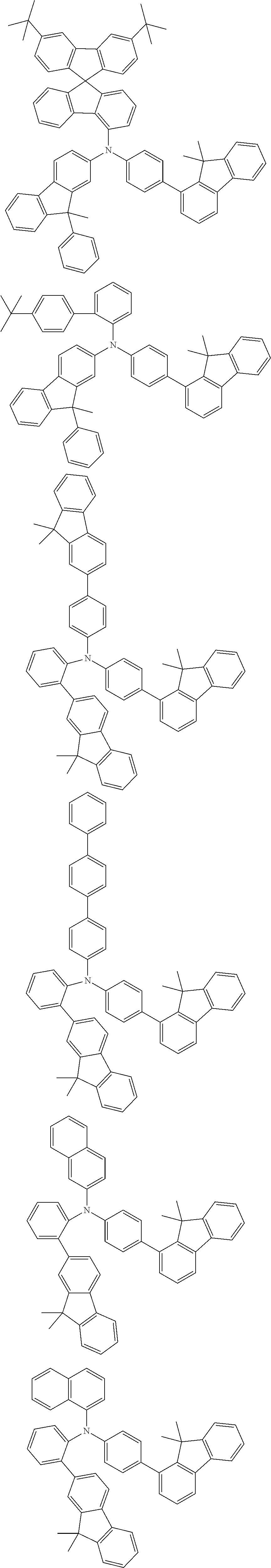

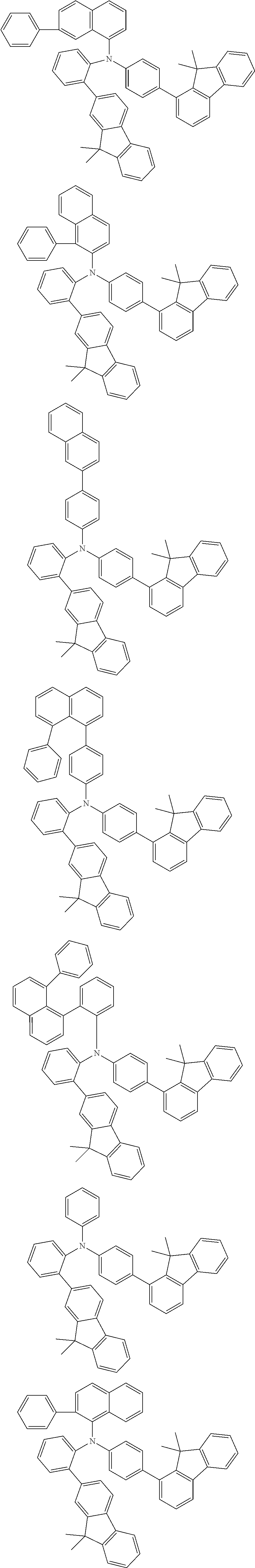

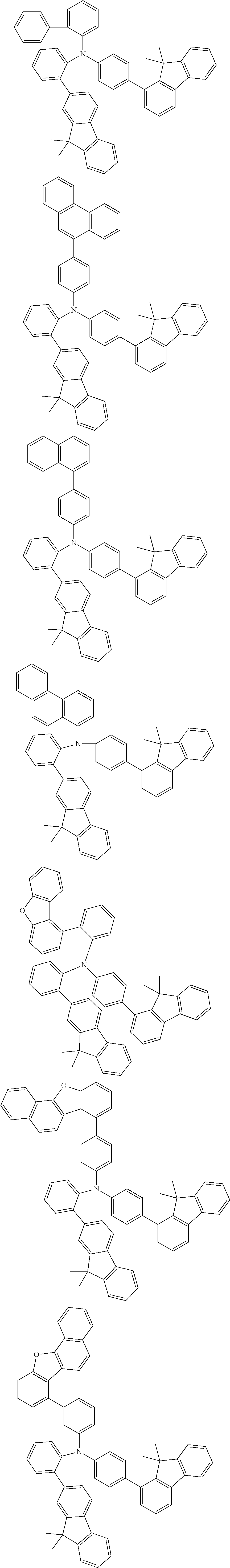

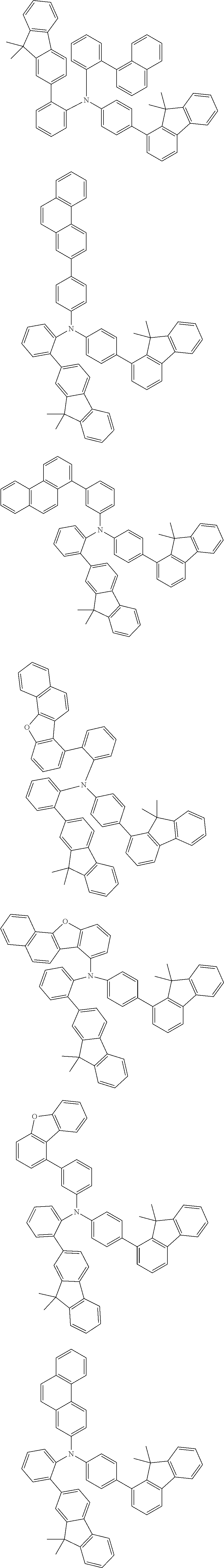

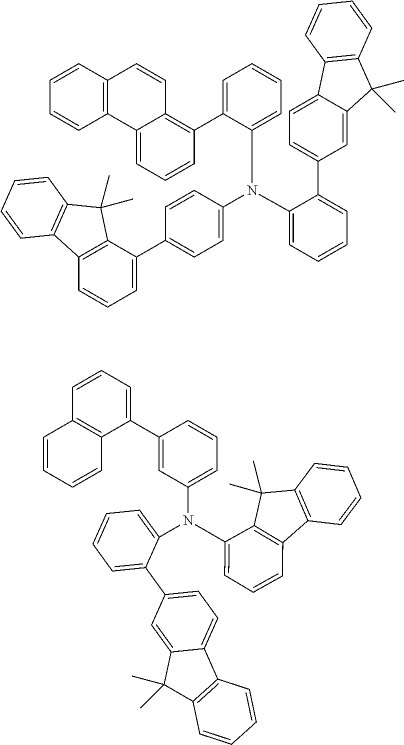 [SMILES: CC1(C)c2ccccc2-c2ccc(-c3ccccc3N(c3ccc(-c4cccc5c4C(C)(C)c4ccccc4-5)cc3)c3ccccc3-c3cccc4c3ccc3ccccc34)cc21 ; CC1(C)c2ccccc2-c2ccc(-c3ccccc3N(c3cccc(-c4cccc5ccccc45)c3)c3cccc4c3C(C)(C)c3ccccc3-4)cc21]

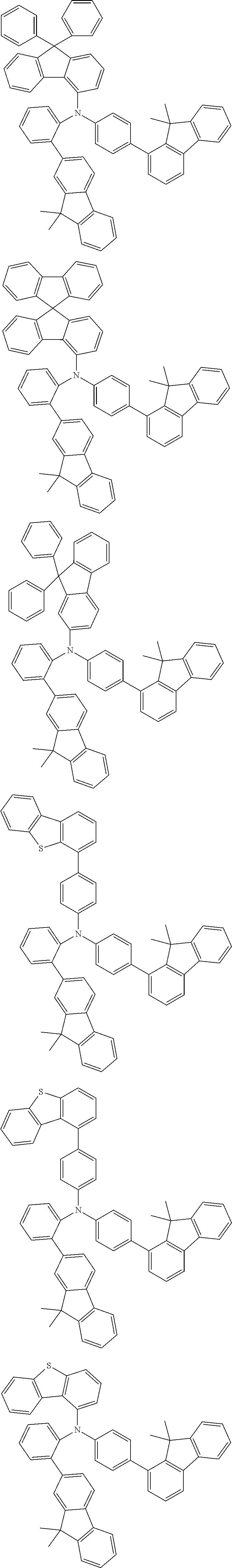

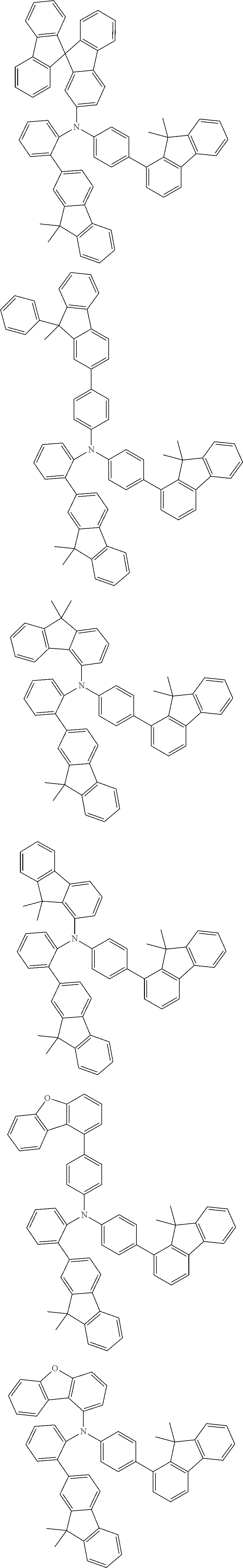

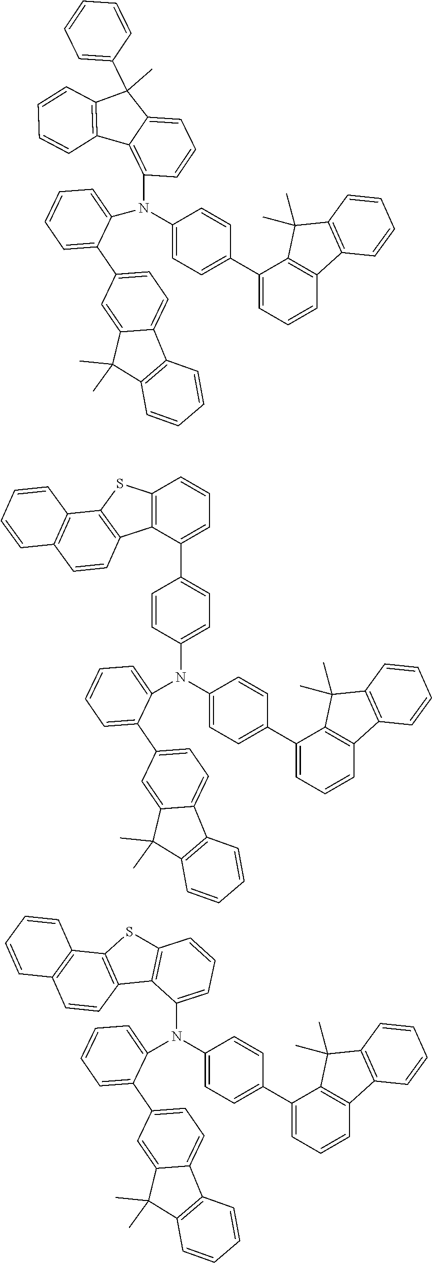

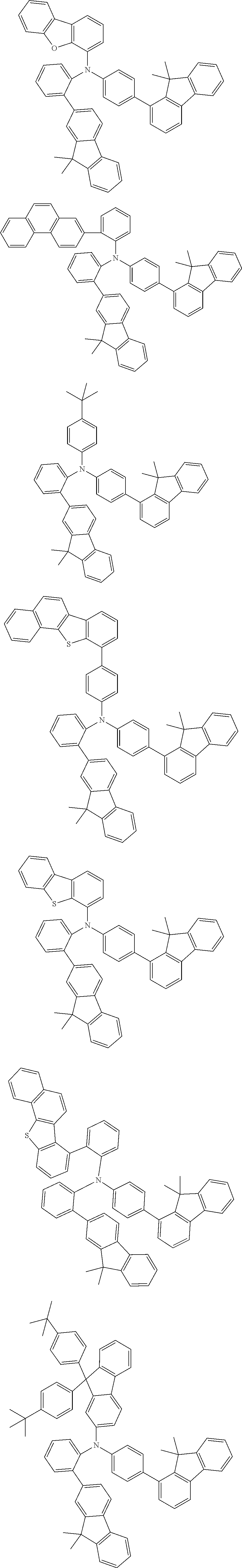

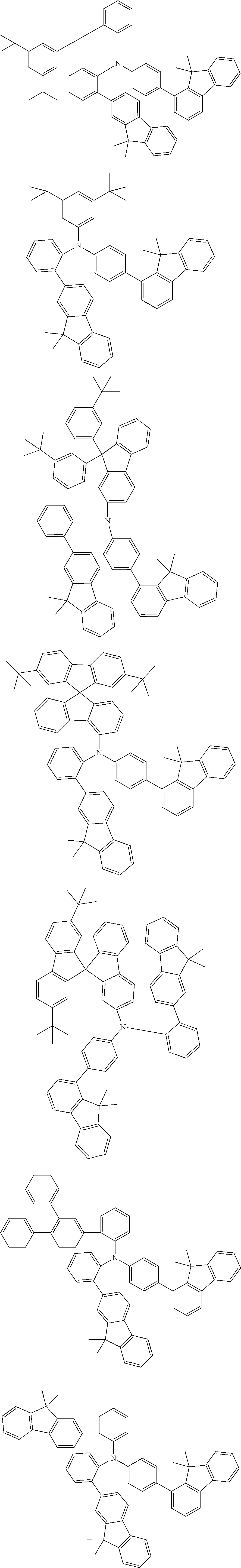

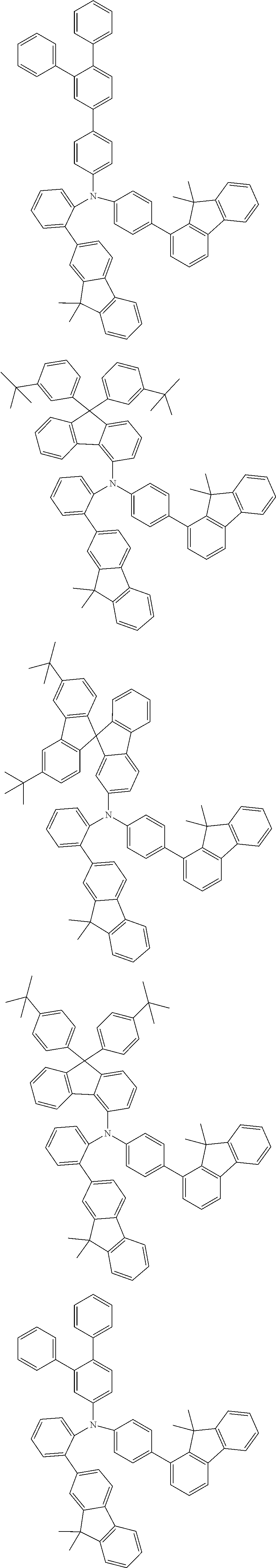

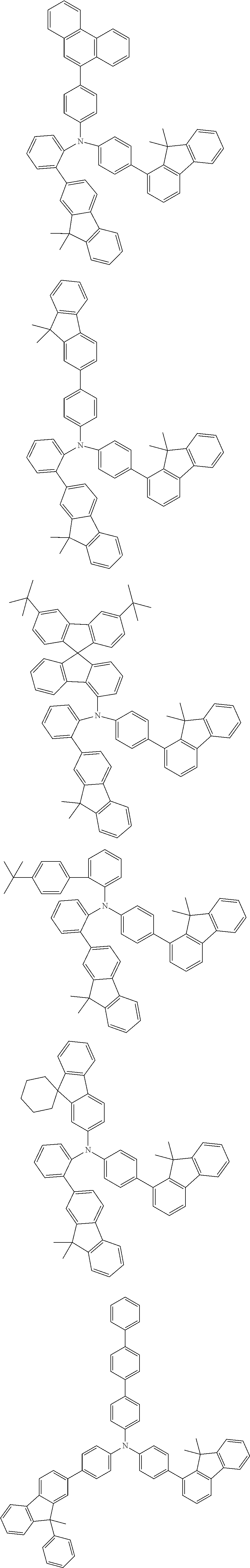

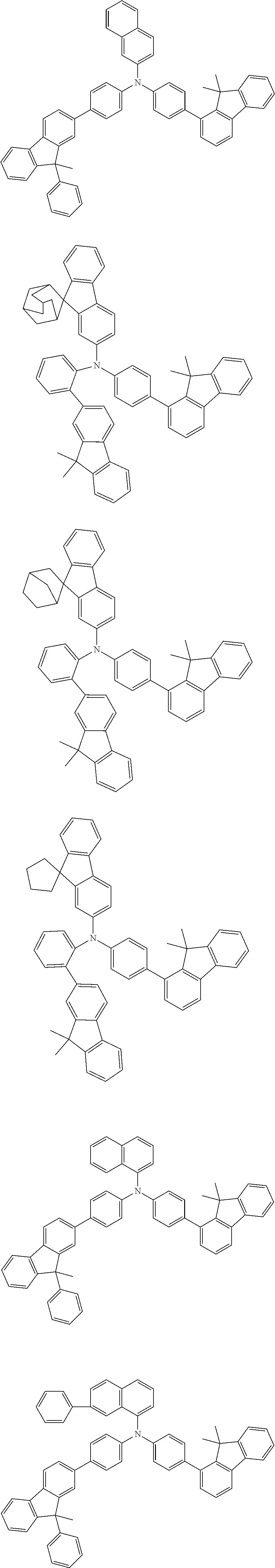

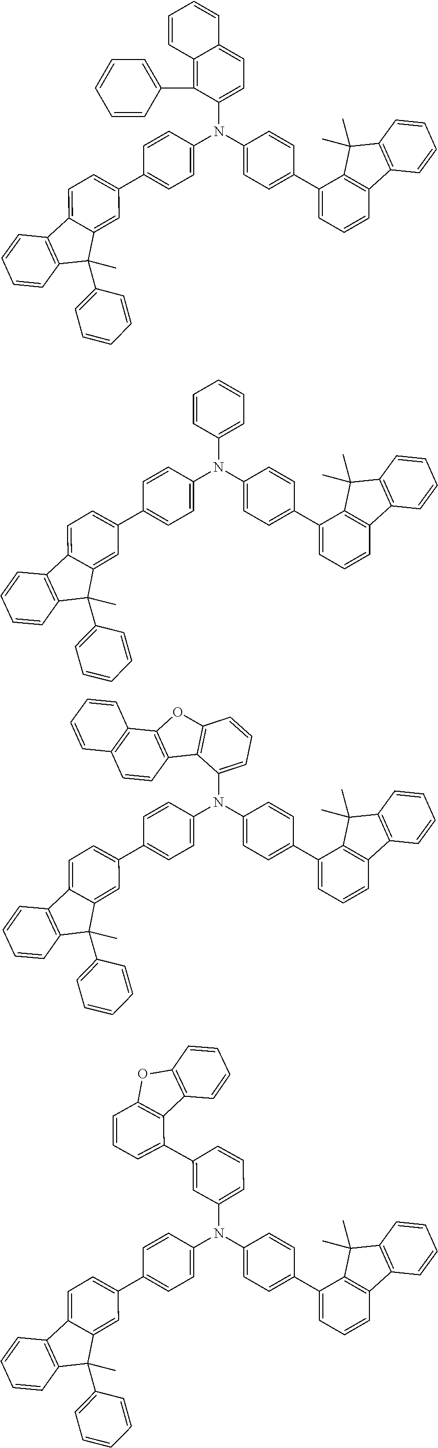

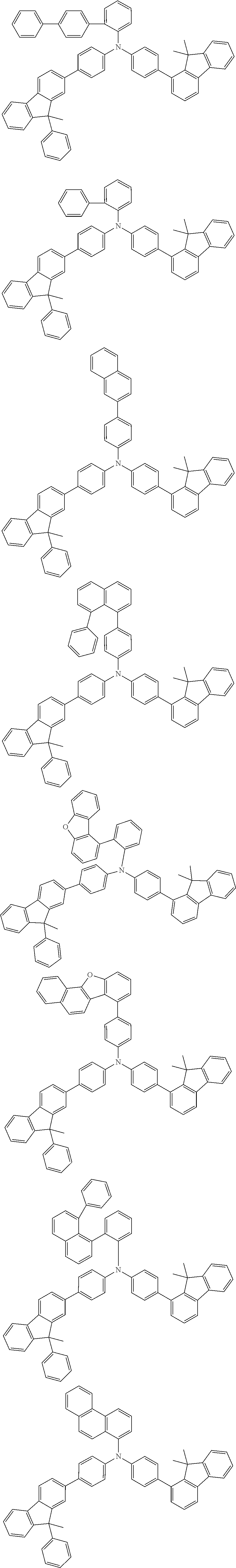

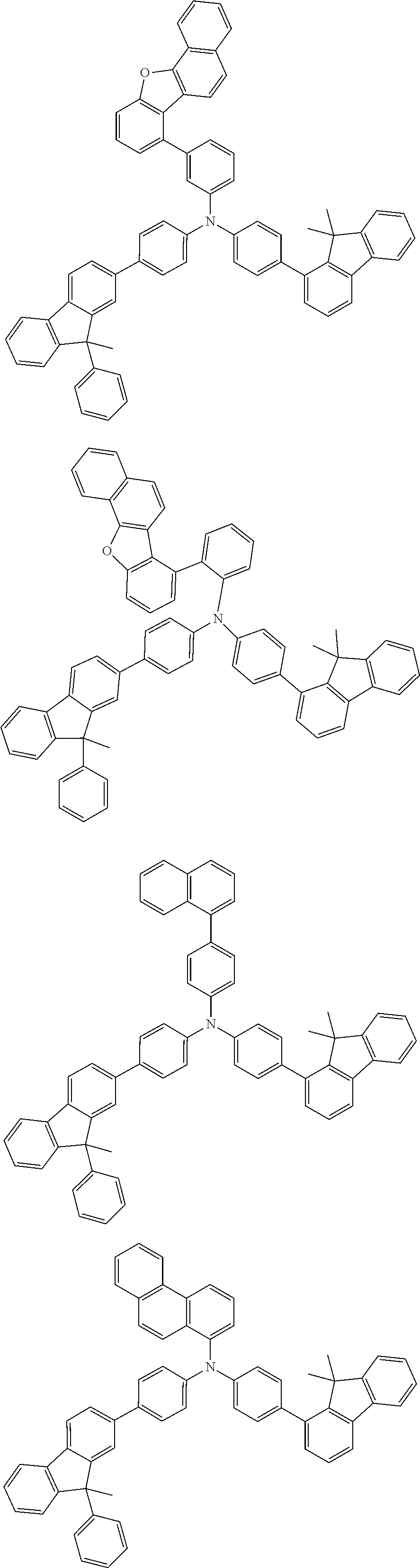

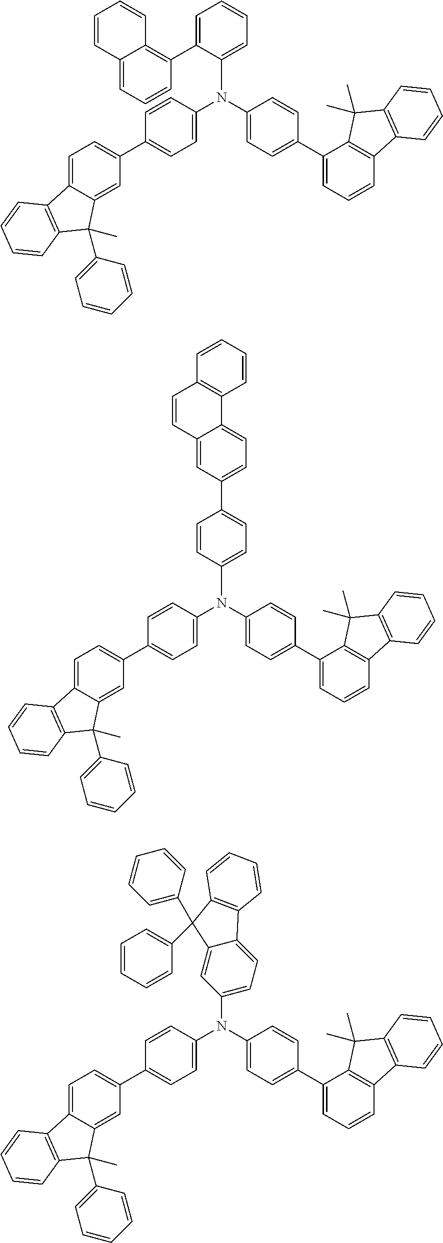 [SMILES: CC1(C)c2ccccc2-c2cccc(-c3ccc(N(c4ccc(-c5ccc6c(c5)C(C)(c5ccccc5)c5ccccc5-6)cc4)c4ccccc4-c4cccc5ccccc45)cc3)c21 ; CC1(C)c2ccccc2-c2cccc(-c3ccc(N(c4ccc(-c5ccc6c(c5)C(C)(c5ccccc5)c5ccccc5-6)cc4)c4ccc(-c5ccc6c(ccc7ccccc76)c5)cc4)cc3)c21 ; CC1(C)c2ccccc2-c2cccc(-c3ccc(N(c4ccc(-c5ccc6c(c5)C(C)(c5ccccc5)c5ccccc5-6)cc4)c4ccc5c(c4)C(c4ccccc4)(c4ccccc4)c4ccccc4-5)cc3)c21]

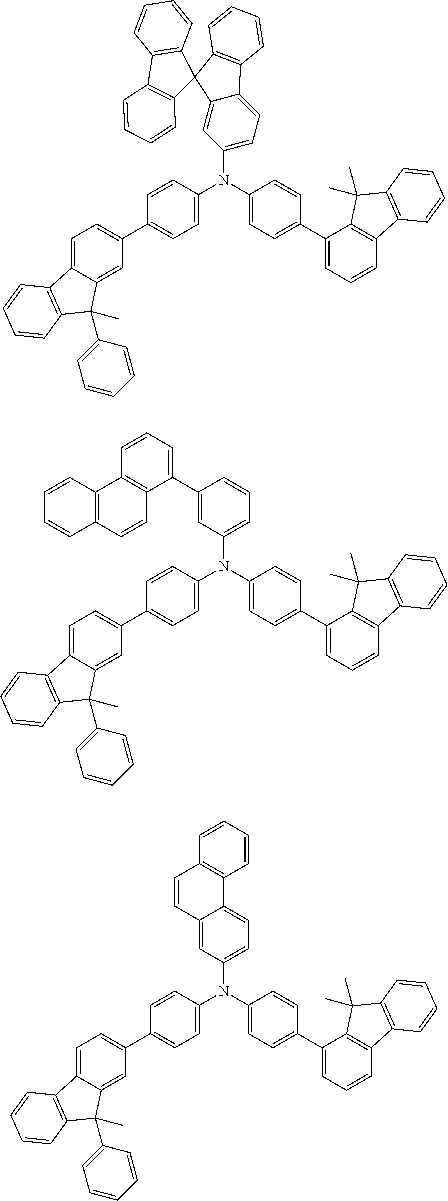 [SMILES: CC1(C)c2ccccc2-c2cccc(-c3ccc(N(c4ccc(-c5ccc6c(c5)C(C)(c5ccccc5)c5ccccc5-6)cc4)c4ccc5c(c4)C4(c6ccccc6-c6ccccc64)c4ccccc4-5)cc3)c21 ; CC1(C)c2ccccc2-c2cccc(-c3ccc(N(c4ccc(-c5ccc6c(c5)C(C)(c5ccccc5)c5ccccc5-6)cc4)c4cccc(-c5cccc6c5ccc5ccccc56)c4)cc3)c21 ; CC1(C)c2ccccc2-c2cccc(-c3ccc(N(c4ccc(-c5ccc6c(c5)C(C)(c5ccccc5)c5ccccc5-6)cc4)c4ccc5c(ccc6ccccc65)c4)cc3)c21]

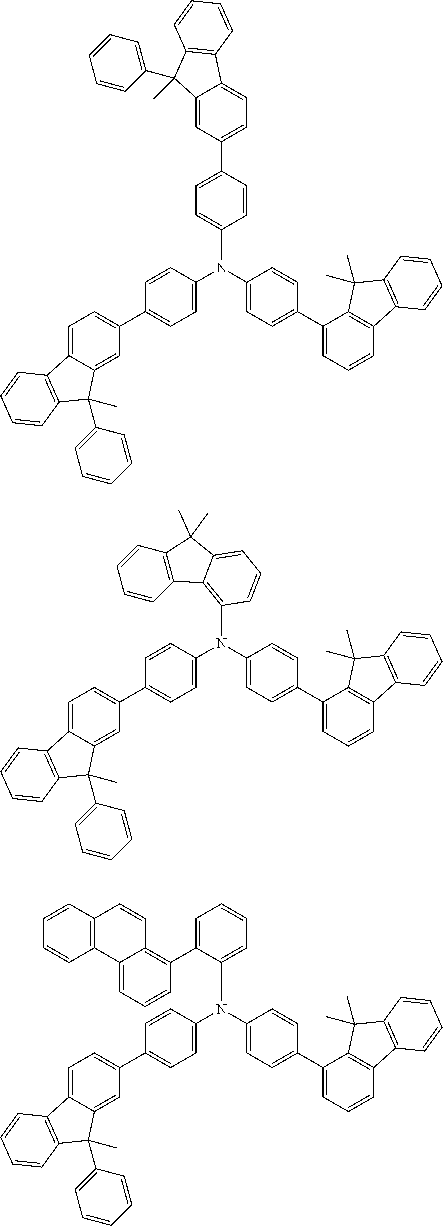 [SMILES: CC1(C)c2ccccc2-c2cccc(-c3ccc(N(c4ccc(-c5ccc6c(c5)C(C)(c5ccccc5)c5ccccc5-6)cc4)c4ccc(-c5ccc6c(c5)C(C)(c5ccccc5)c5ccccc5-6)cc4)cc3)c21 ; CC1(C)c2ccccc2-c2c(N(c3ccc(-c4ccc5c(c4)C(C)(c4ccccc4)c4ccccc4-5)cc3)c3ccc(-c4cccc5c4C(C)(C)c4ccccc4-5)cc3)cccc21 ; CC1(C)c2ccccc2-c2cccc(-c3ccc(N(c4ccc(-c5ccc6c(c5)C(C)(c5ccccc5)c5ccccc5-6)cc4)c4ccccc4-c4cccc5c4ccc4ccccc45)cc3)c21]

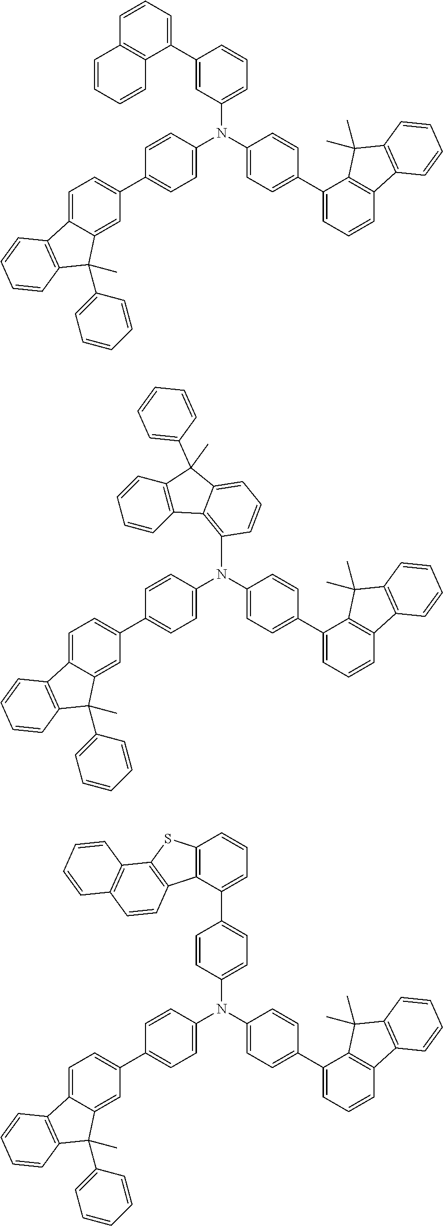 [SMILES: CC1(C)c2ccccc2-c2cccc(-c3ccc(N(c4ccc(-c5ccc6c(c5)C(C)(c5ccccc5)c5ccccc5-6)cc4)c4cccc(-c5cccc6ccccc56)c4)cc3)c21 ; CC1(C)c2ccccc2-c2cccc(-c3ccc(N(c4ccc(-c5ccc6c(c5)C(C)(c5ccccc5)c5ccccc5-6)cc4)c4cccc5c4-c4ccccc4C5(C)c4ccccc4)cc3)c21 ; CC1(C)c2ccccc2-c2cccc(-c3ccc(N(c4ccc(-c5ccc6c(c5)C(C)(c5ccccc5)c5ccccc5-6)cc4)c4ccc(-c5cccc6sc7c8ccccc8ccc7c56)cc4)cc3)c21]

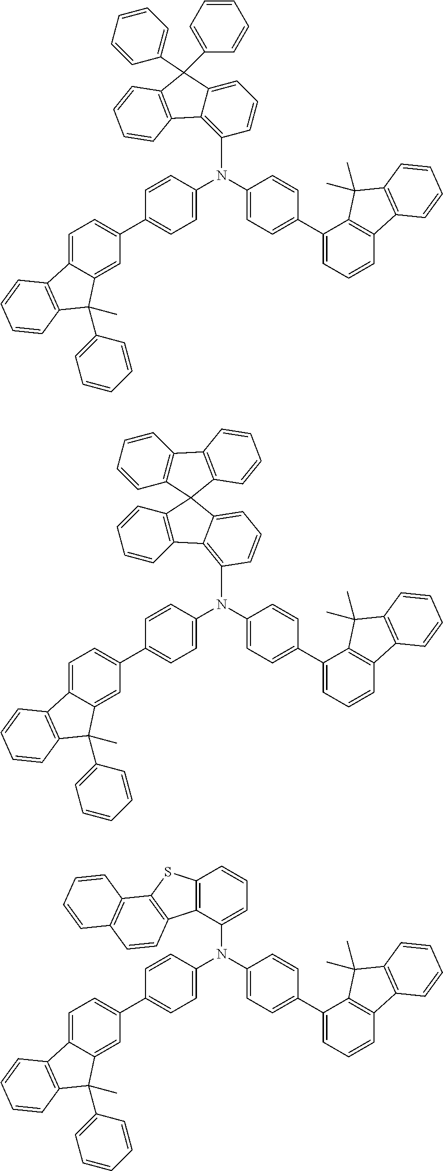 [SMILES: CC1(C)c2ccccc2-c2cccc(-c3ccc(N(c4ccc(-c5ccc6c(c5)C(C)(c5ccccc5)c5ccccc5-6)cc4)c4cccc5c4-c4ccccc4C5(c4ccccc4)c4ccccc4)cc3)c21 ; CC1(C)c2ccccc2-c2cccc(-c3ccc(N(c4ccc(-c5ccc6c(c5)C(C)(c5ccccc5)c5ccccc5-6)cc4)c4cccc5c4-c4ccccc4C54c5ccccc5-c5ccccc54)cc3)c21 ; CC1(C)c2ccccc2-c2cccc(-c3ccc(N(c4ccc(-c5ccc6c(c5)C(C)(c5ccccc5)c5ccccc5-6)cc4)c4cccc5sc6c7ccccc7ccc6c45)cc3)c21]

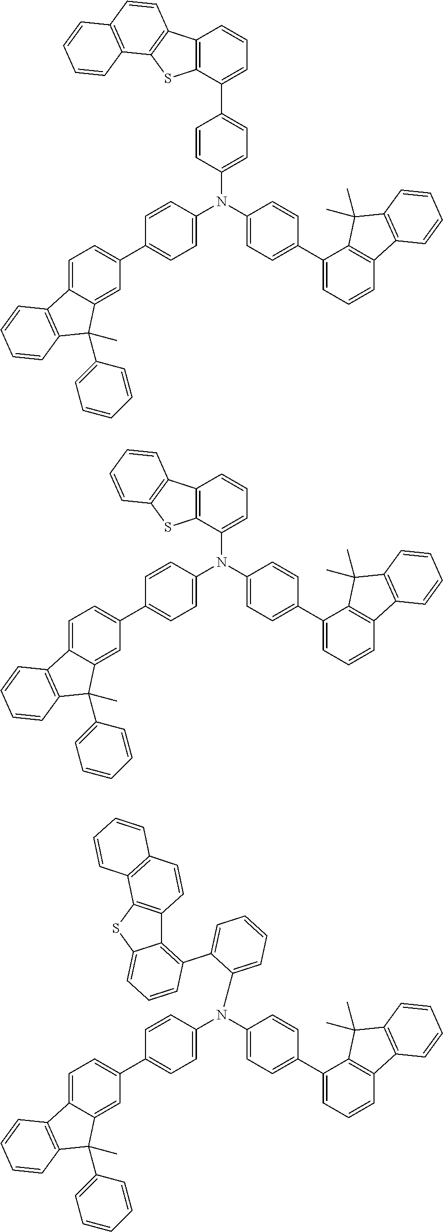

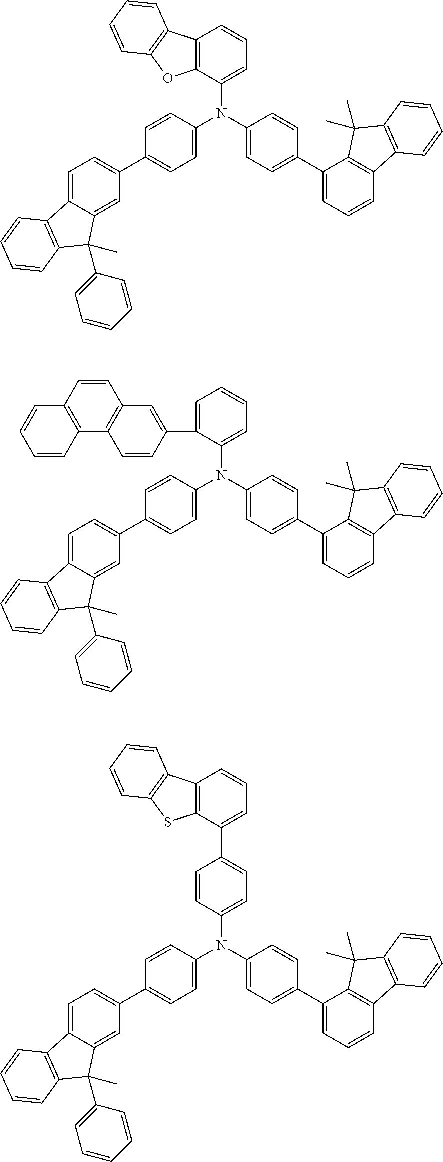 [SMILES: CC1(C)c2ccccc2-c2cccc(-c3ccc(N(c4ccc(-c5ccc6c(c5)C(C)(c5ccccc5)c5ccccc5-6)cc4)c4cccc5c4oc4ccccc45)cc3)c21 ; CC1(C)c2ccccc2-c2cccc(-c3ccc(N(c4ccc(-c5ccc6c(c5)C(C)(c5ccccc5)c5ccccc5-6)cc4)c4ccccc4-c4ccc5c(ccc6ccccc65)c4)cc3)c21 ; CC1(C)c2ccccc2-c2cccc(-c3ccc(N(c4ccc(-c5ccc6c(c5)C(C)(c5ccccc5)c5ccccc5-6)cc4)c4ccc(-c5cccc6c5sc5ccccc56)cc4)cc3)c21]

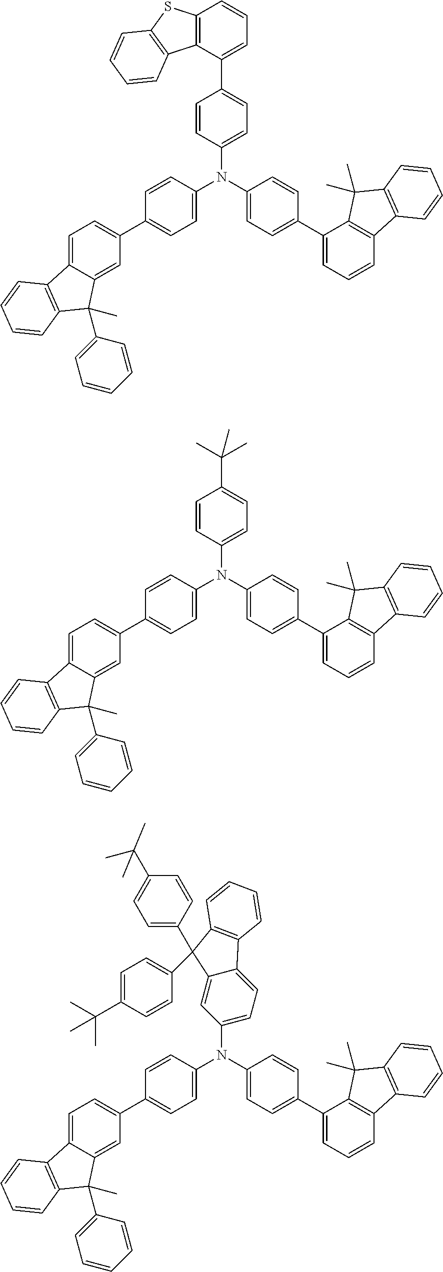 [SMILES: CC1(C)c2ccccc2-c2cccc(-c3ccc(N(c4ccc(-c5ccc6c(c5)C(C)(c5ccccc5)c5ccccc5-6)cc4)c4ccc(-c5cccc6sc7ccccc7c56)cc4)cc3)c21 ; CC(C)(C)c1ccc(N(c2ccc(-c3ccc4c(c3)C(C)(c3ccccc3)c3ccccc3-4)cc2)c2ccc(-c3cccc4c3C(C)(C)c3ccccc3-4)cc2)cc1 ; CC(C)(C)c1ccc(C2(c3ccc(C(C)(C)C)cc3)c3ccccc3-c3ccc(N(c4ccc(-c5ccc6c(c5)C(C)(c5ccccc5)c5ccccc5-6)cc4)c4ccc(-c5cccc6c5C(C)(C)c5ccccc5-6)cc4)cc32)cc1]

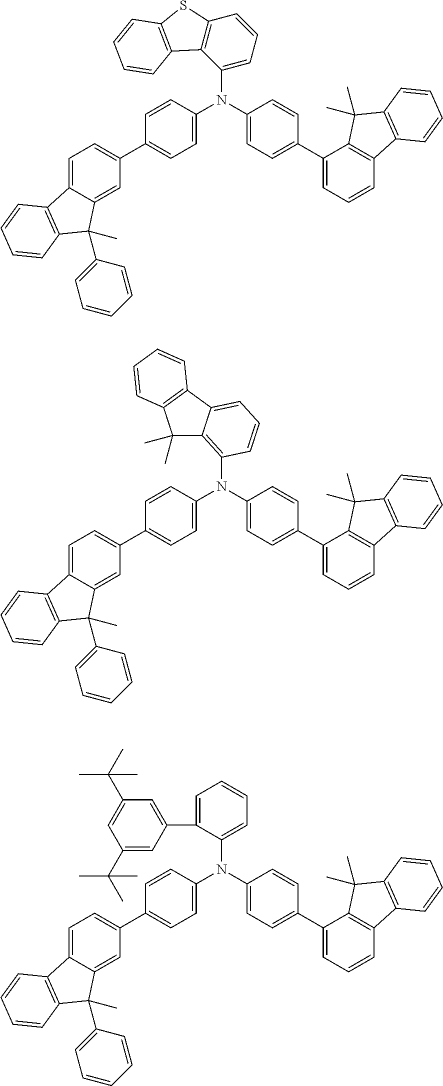 [SMILES: CC1(C)c2ccccc2-c2cccc(-c3ccc(N(c4ccc(-c5ccc6c(c5)C(C)(c5ccccc5)c5ccccc5-6)cc4)c4cccc5sc6ccccc6c45)cc3)c21 ; CC1(C)c2ccccc2-c2cccc(-c3ccc(N(c4ccc(-c5ccc6c(c5)C(C)(c5ccccc5)c5ccccc5-6)cc4)c4cccc5c4C(C)(C)c4ccccc4-5)cc3)c21 ; CC(C)(C)c1cc(-c2ccccc2N(c2ccc(-c3ccc4c(c3)C(C)(c3ccccc3)c3ccccc3-4)cc2)c2ccc(-c3cccc4c3C(C)(C)c3ccccc3-4)cc2)cc(C(C)(C)C)c1]

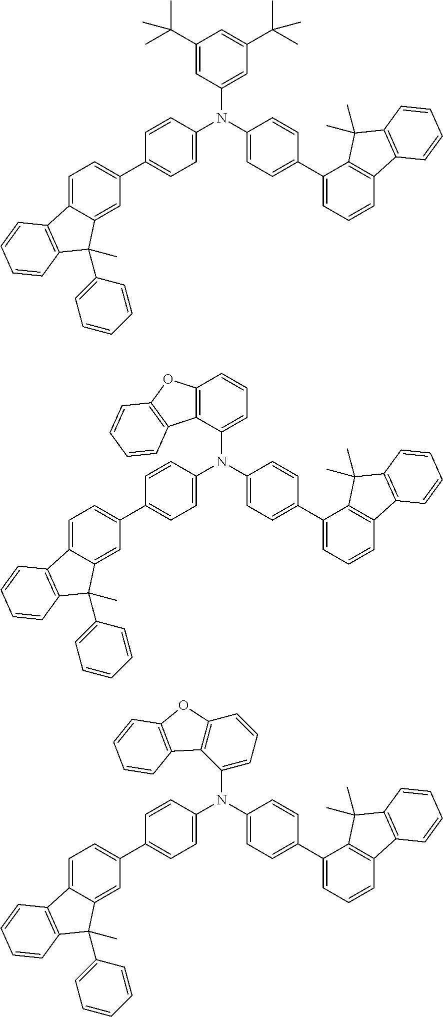 [SMILES: CC(C)(C)c1cc(N(c2ccc(-c3ccc4c(c3)C(C)(c3ccccc3)c3ccccc3-4)cc2)c2ccc(-c3cccc4c3C(C)(C)c3ccccc3-4)cc2)cc(C(C)(C)C)c1 ; CC1(C)c2ccccc2-c2cccc(-c3ccc(N(c4ccc(-c5ccc6c(c5)C(C)(c5ccccc5)c5ccccc5-6)cc4)c4cccc5oc6ccccc6c45)cc3)c21 ; CC1(C)c2ccccc2-c2cccc(-c3ccc(N(c4ccc(-c5ccc6c(c5)C(C)(c5ccccc5)c5ccccc5-6)cc4)c4cccc5oc6ccccc6c45)cc3)c21]

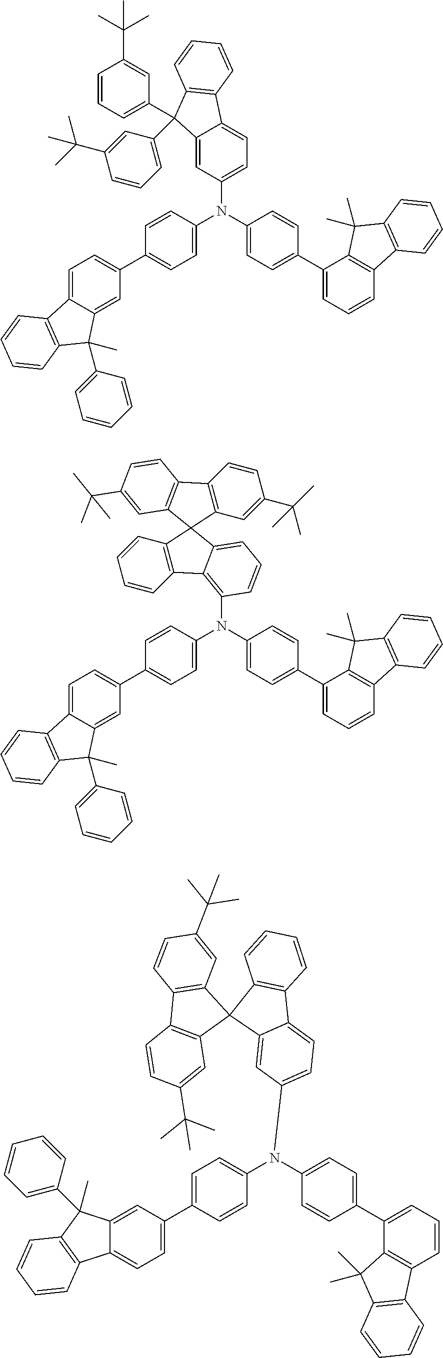 [SMILES: CC(C)(C)c1cccc(C2(c3cccc(C(C)(C)C)c3)c3ccccc3-c3ccc(N(c4ccc(-c5ccc6c(c5)C(C)(c5ccccc5)c5ccccc5-6)cc4)c4ccc(-c5cccc6c5C(C)(C)c5ccccc5-6)cc4)cc32)c1 ; CC(C)(C)c1ccc2c(c1)C1(c3cc(C(C)(C)C)ccc3-2)c2ccccc2-c2c(N(c3ccc(-c4ccc5c(c4)C(C)(c4ccccc4)c4ccccc4-5)cc3)c3ccc(-c4cccc5c4C(C)(C)c4ccccc4-5)cc3)cccc21 ; CC(C)(C)c1ccc2c(c1)C1(c3ccccc3-c3ccc(N(c4ccc(-c5ccc6c(c5)C(C)(c5ccccc5)c5ccccc5-6)cc4)c4ccc(-c5cccc6c5C(C)(C)c5ccccc5-6)cc4)cc31)c1cc(C(C)(C)C)ccc1-2]

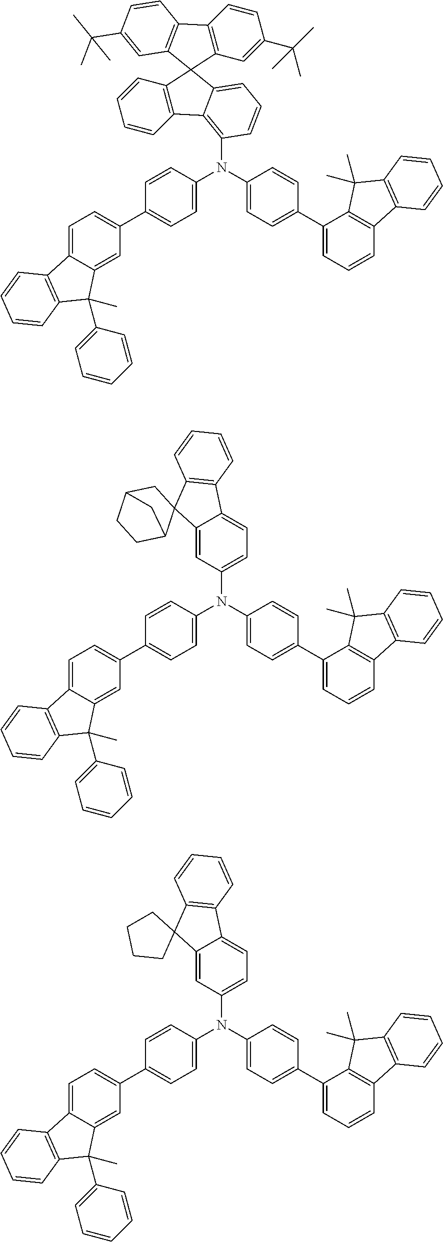 [SMILES: CC(C)(C)c1ccc2c(c1)C1(c3cc(C(C)(C)C)ccc3-2)c2ccccc2-c2c(N(c3ccc(-c4ccc5c(c4)C(C)(c4ccccc4)c4ccccc4-5)cc3)c3ccc(-c4cccc5c4C(C)(C)c4ccccc4-5)cc3)cccc21 ; CC1(C)c2ccccc2-c2cccc(-c3ccc(N(c4ccc(-c5ccc6c(c5)C(C)(c5ccccc5)c5ccccc5-6)cc4)c4ccc5c(c4)C4(CC6CCC4C6)c4ccccc4-5)cc3)c21 ; CC1(C)c2ccccc2-c2cccc(-c3ccc(N(c4ccc(-c5ccc6c(c5)C(C)(c5ccccc5)c5ccccc5-6)cc4)c4ccc5c(c4)C4(CCCC4)c4ccccc4-5)cc3)c21]

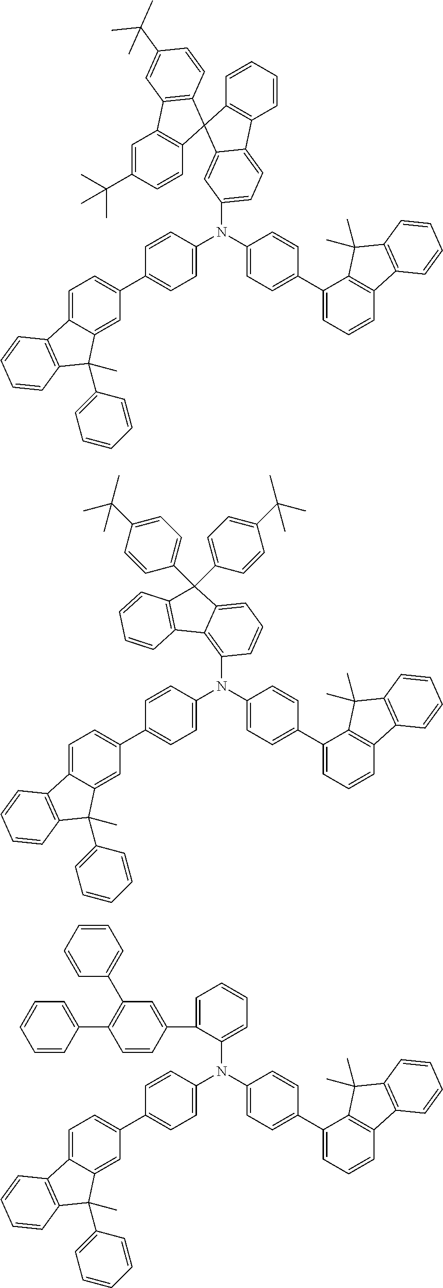

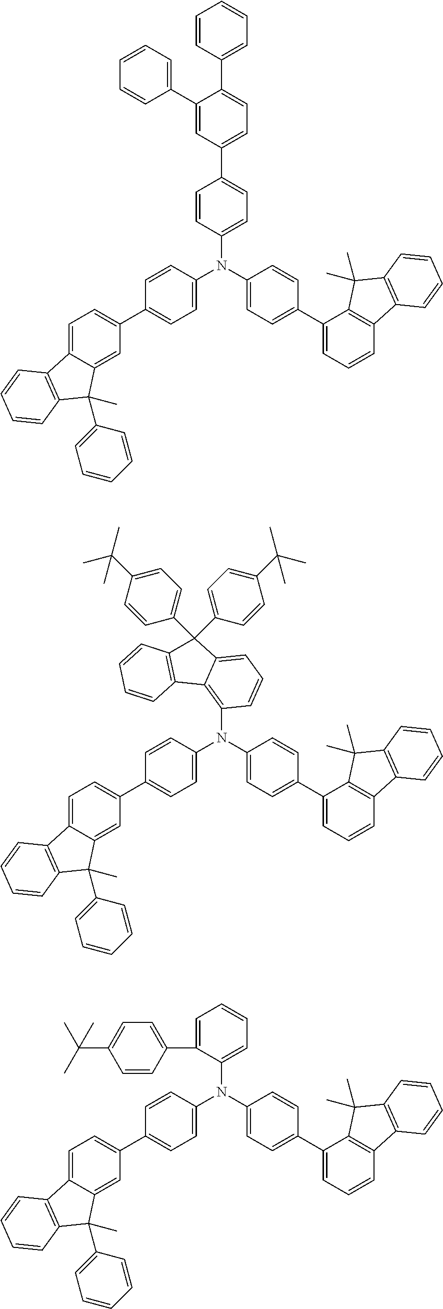 [SMILES: CC1(C)c2ccccc2-c2cccc(-c3ccc(N(c4ccc(-c5ccc(-c6ccccc6)c(-c6ccccc6)c5)cc4)c4ccc(-c5ccc6c(c5)C(C)(c5ccccc5)c5ccccc5-6)cc4)cc3)c21 ; CC(C)(C)c1ccc(C2(c3ccc(C(C)(C)C)cc3)c3ccccc3-c3c(N(c4ccc(-c5ccc6c(c5)C(C)(c5ccccc5)c5ccccc5-6)cc4)c4ccc(-c5cccc6c5C(C)(C)c5ccccc5-6)cc4)cccc32)cc1 ; CC(C)(C)c1ccc(-c2ccccc2N(c2ccc(-c3ccc4c(c3)C(C)(c3ccccc3)c3ccccc3-4)cc2)c2ccc(-c3cccc4c3C(C)(C)c3ccccc3-4)cc2)cc1]

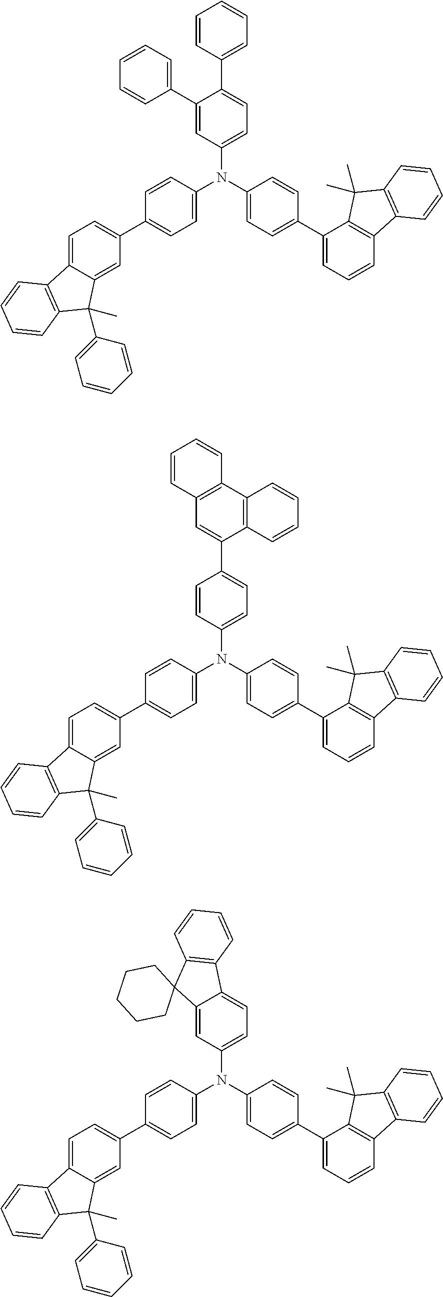 [SMILES: CC1(C)c2ccccc2-c2cccc(-c3ccc(N(c4ccc(-c5ccc6c(c5)C(C)(c5ccccc5)c5ccccc5-6)cc4)c4ccc(-c5ccccc5)c(-c5ccccc5)c4)cc3)c21 ; CC1(C)c2ccccc2-c2cccc(-c3ccc(N(c4ccc(-c5ccc6c(c5)C(C)(c5ccccc5)c5ccccc5-6)cc4)c4ccc(-c5cc6ccccc6c6ccccc56)cc4)cc3)c21 ; CC1(C)c2ccccc2-c2cccc(-c3ccc(N(c4ccc(-c5ccc6c(c5)C(C)(c5ccccc5)c5ccccc5-6)cc4)c4ccc5c(c4)C4(CCCCC4)c4ccccc4-5)cc3)c21]

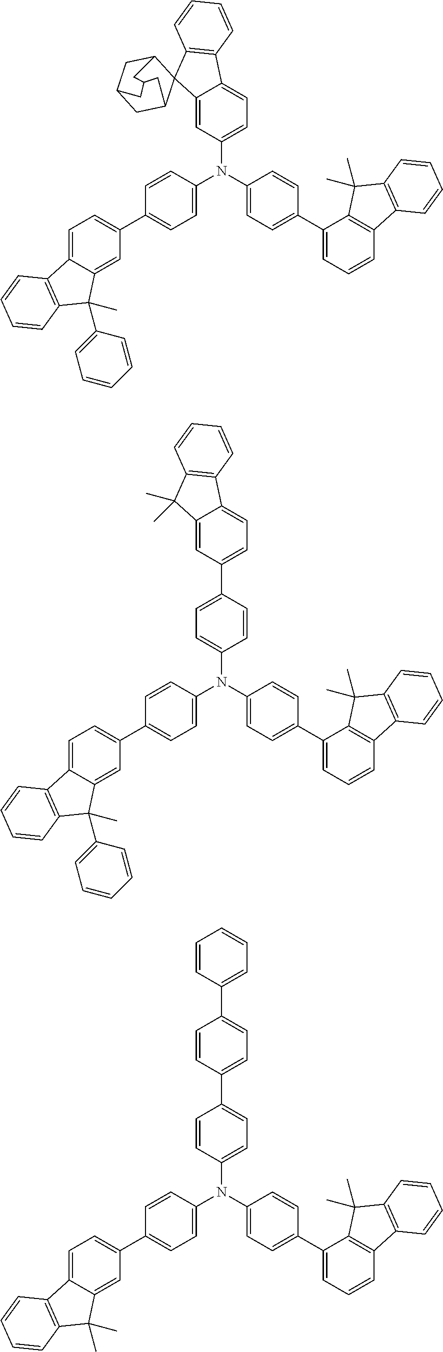 [SMILES: CC1(C)c2ccccc2-c2cccc(-c3ccc(N(c4ccc(-c5ccc6c(c5)C(C)(c5ccccc5)c5ccccc5-6)cc4)c4ccc5c(c4)C4(c6ccccc6-5)C5CC6CC(C5)CC4C6)cc3)c21 ; CC1(C)c2ccccc2-c2ccc(-c3ccc(N(c4ccc(-c5ccc6c(c5)C(C)(c5ccccc5)c5ccccc5-6)cc4)c4ccc(-c5cccc6c5C(C)(C)c5ccccc5-6)cc4)cc3)cc21 ; CC1(C)c2ccccc2-c2ccc(-c3ccc(N(c4ccc(-c5ccc(-c6ccccc6)cc5)cc4)c4ccc(-c5cccc6c5C(C)(C)c5ccccc5-6)cc4)cc3)cc21]

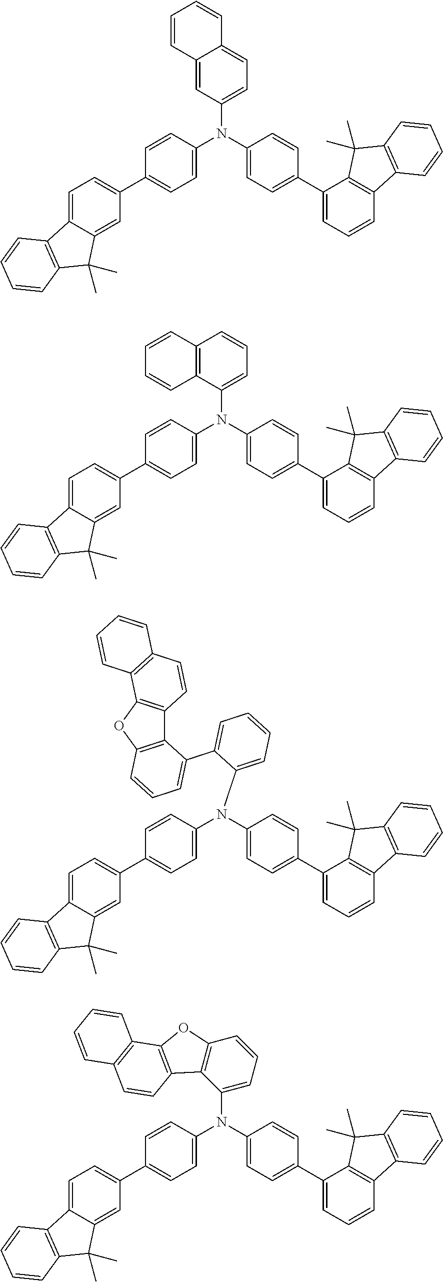

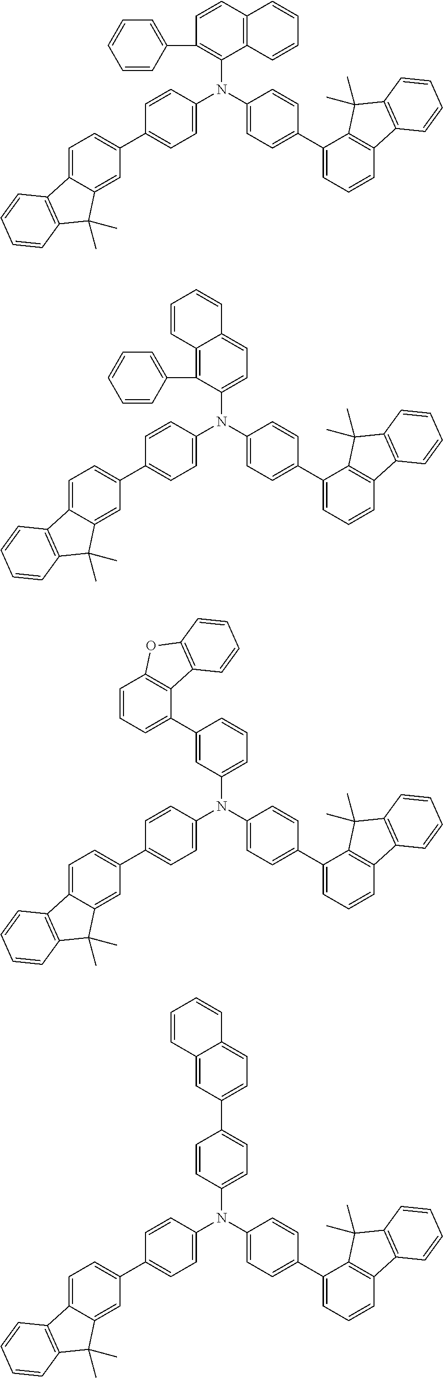 [SMILES: CC1(C)c2ccccc2-c2ccc(-c3ccc(N(c4ccc(-c5cccc6c5C(C)(C)c5ccccc5-6)cc4)c4c(-c5ccccc5)ccc5ccccc45)cc3)cc21 ; CC1(C)c2ccccc2-c2ccc(-c3ccc(N(c4ccc(-c5cccc6c5C(C)(C)c5ccccc5-6)cc4)c4ccc5ccccc5c4-c4ccccc4)cc3)cc21 ; CC1(C)c2ccccc2-c2ccc(-c3ccc(N(c4ccc(-c5cccc6c5C(C)(C)c5ccccc5-6)cc4)c4cccc(-c5cccc6oc7ccccc7c56)c4)cc3)cc21 ; CC1(C)c2ccccc2-c2ccc(-c3ccc(N(c4ccc(-c5ccc6ccccc6c5)cc4)c4ccc(-c5cccc6c5C(C)(C)c5ccccc5-6)cc4)cc3)cc21]

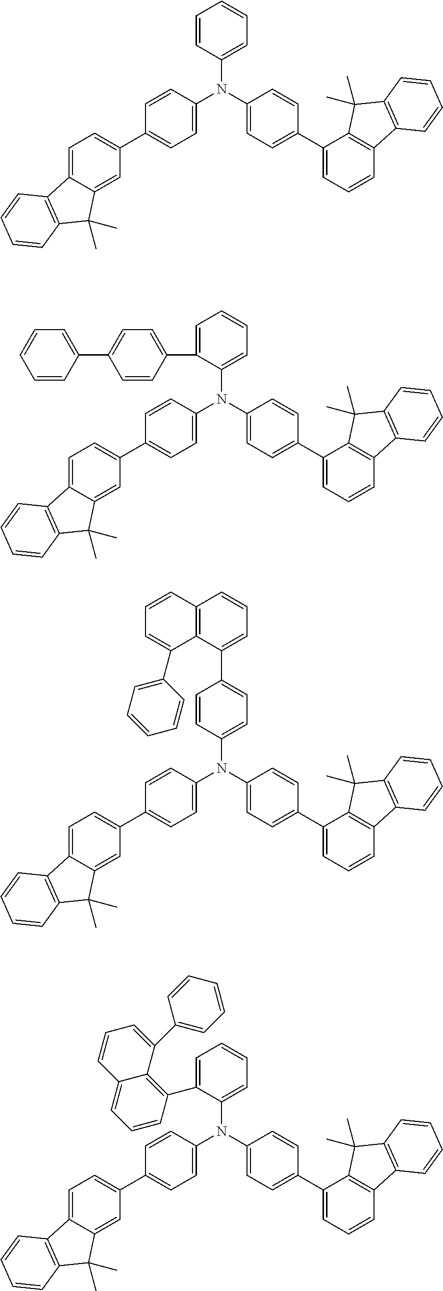 [SMILES: CC1(C)c2ccccc2-c2ccc(-c3ccc(N(c4ccccc4)c4ccc(-c5cccc6c5C(C)(C)c5ccccc5-6)cc4)cc3)cc21 ; CC1(C)c2ccccc2-c2ccc(-c3ccc(N(c4ccc(-c5cccc6c5C(C)(C)c5ccccc5-6)cc4)c4ccccc4-c4ccc(-c5ccccc5)cc4)cc3)cc21 ; CC1(C)c2ccccc2-c2ccc(-c3ccc(N(c4ccc(-c5cccc6c5C(C)(C)c5ccccc5-6)cc4)c4ccc(-c5cccc6cccc(-c7ccccc7)c56)cc4)cc3)cc21 ; CC1(C)c2ccccc2-c2ccc(-c3ccc(N(c4ccc(-c5cccc6c5C(C)(C)c5ccccc5-6)cc4)c4ccccc4-c4cccc5cccc(-c6ccccc6)c45)cc3)cc21]

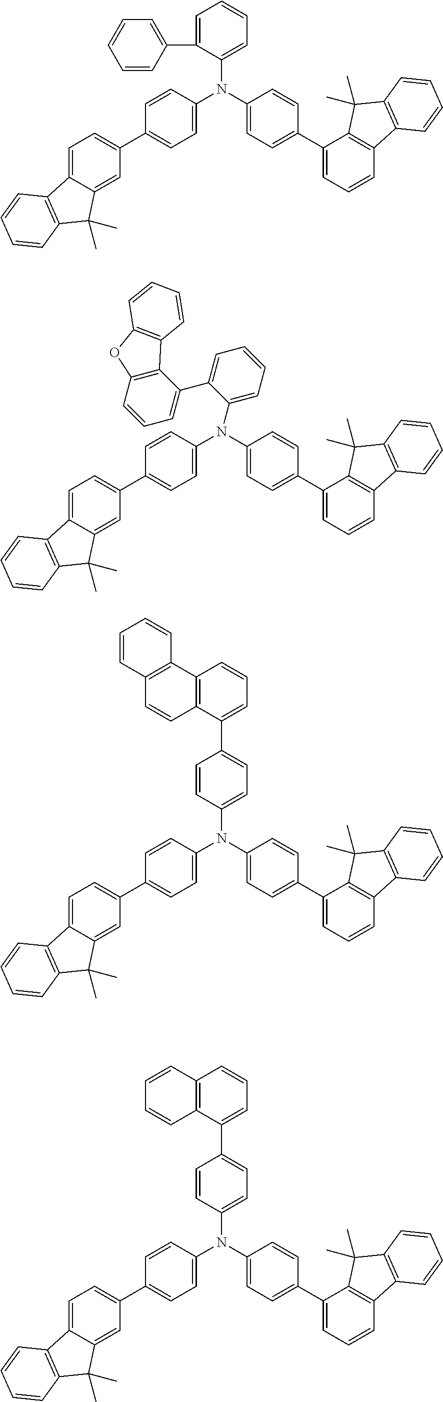 [SMILES: CC1(C)c2ccccc2-c2ccc(-c3ccc(N(c4ccc(-c5cccc6c5C(C)(C)c5ccccc5-6)cc4)c4ccccc4-c4ccccc4)cc3)cc21 ; CC1(C)c2ccccc2-c2ccc(-c3ccc(N(c4ccc(-c5cccc6c5C(C)(C)c5ccccc5-6)cc4)c4ccccc4-c4cccc5oc6ccccc6c45)cc3)cc21 ; CC1(C)c2ccccc2-c2ccc(-c3ccc(N(c4ccc(-c5cccc6c5C(C)(C)c5ccccc5-6)cc4)c4ccc(-c5cccc6c5ccc5ccccc56)cc4)cc3)cc21 ; CC1(C)c2ccccc2-c2ccc(-c3ccc(N(c4ccc(-c5cccc6c5C(C)(C)c5ccccc5-6)cc4)c4ccc(-c5cccc6ccccc56)cc4)cc3)cc21]

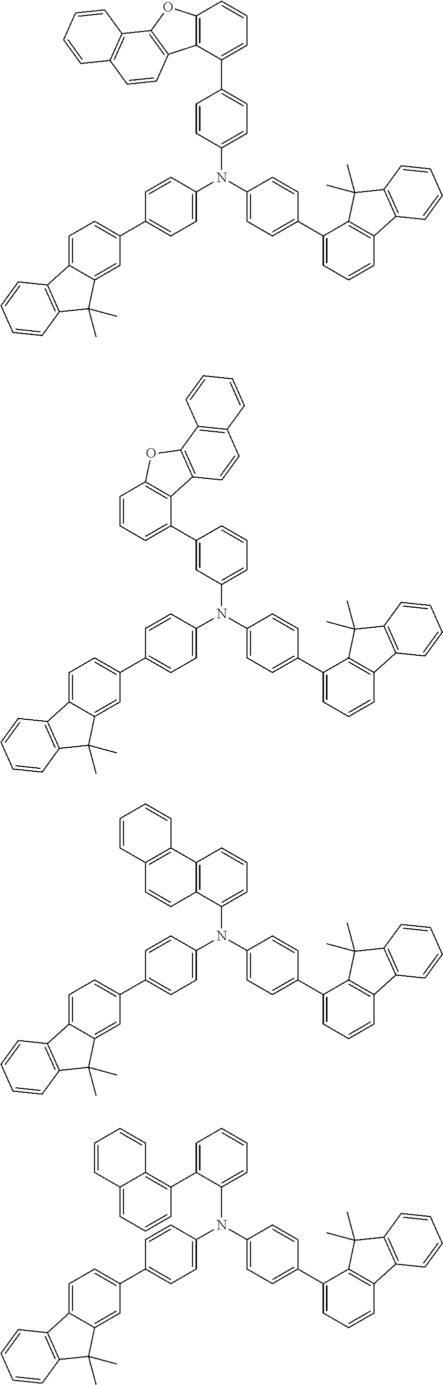

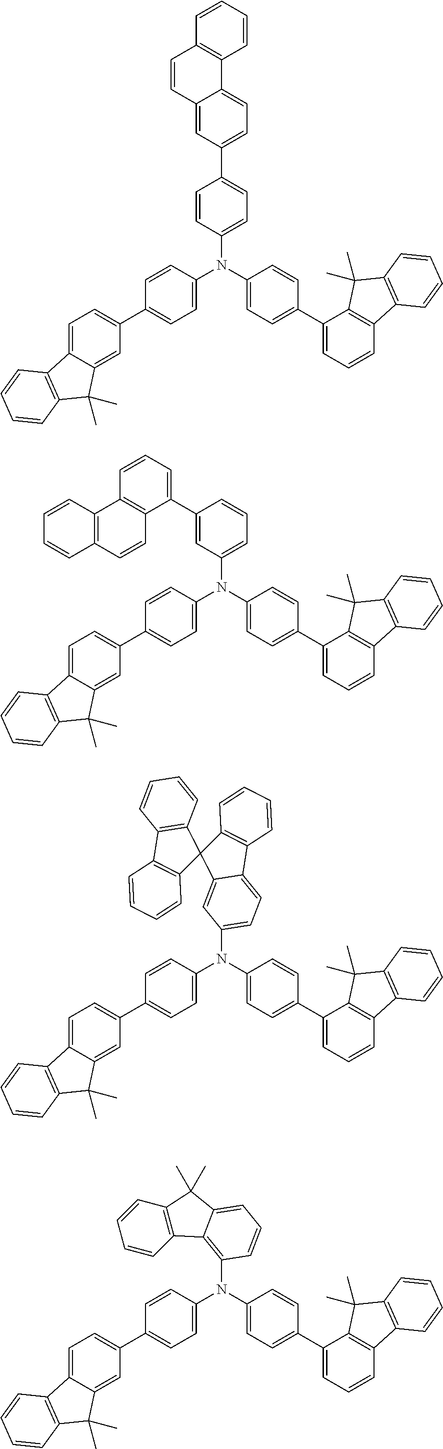 [SMILES: CC1(C)c2ccccc2-c2ccc(-c3ccc(N(c4ccc(-c5ccc6c(ccc7ccccc76)c5)cc4)c4ccc(-c5cccc6c5C(C)(C)c5ccccc5-6)cc4)cc3)cc21 ; CC1(C)c2ccccc2-c2ccc(-c3ccc(N(c4ccc(-c5cccc6c5C(C)(C)c5ccccc5-6)cc4)c4cccc(-c5cccc6c5ccc5ccccc56)c4)cc3)cc21 ; CC1(C)c2ccccc2-c2ccc(-c3ccc(N(c4ccc(-c5cccc6c5C(C)(C)c5ccccc5-6)cc4)c4ccc5c(c4)C4(c6ccccc6-c6ccccc64)c4ccccc4-5)cc3)cc21 ; CC1(C)c2ccccc2-c2ccc(-c3ccc(N(c4ccc(-c5cccc6c5C(C)(C)c5ccccc5-6)cc4)c4cccc5c4-c4ccccc4C5(C)C)cc3)cc21]

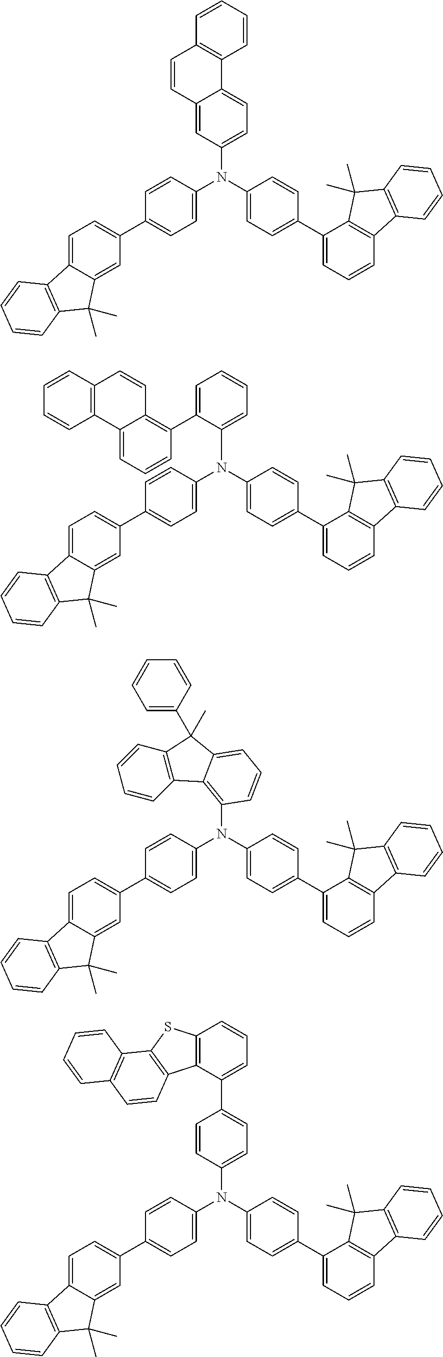

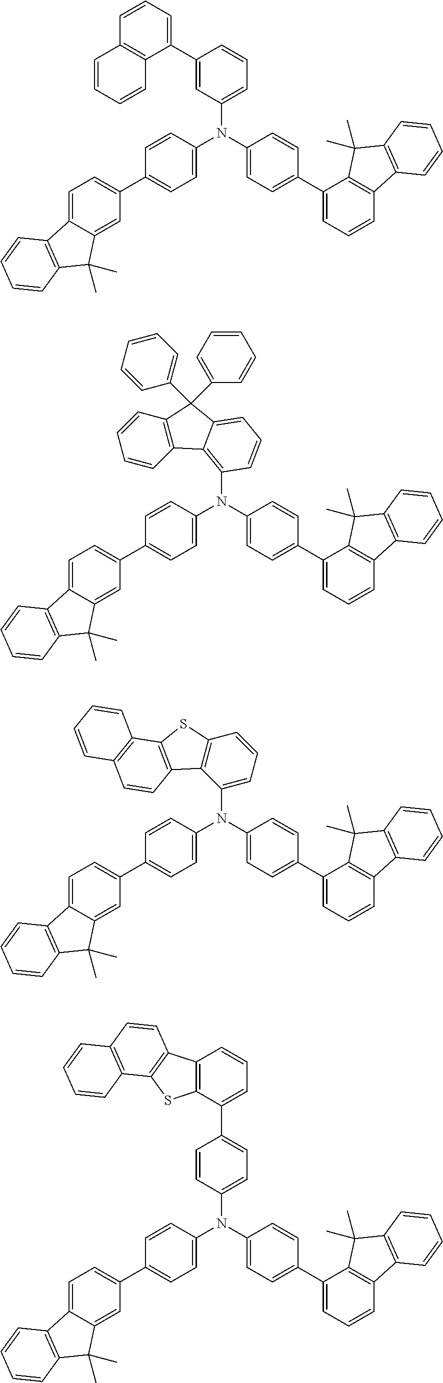 [SMILES: CC1(C)c2ccccc2-c2ccc(-c3ccc(N(c4ccc(-c5cccc6c5C(C)(C)c5ccccc5-6)cc4)c4cccc(-c5cccc6ccccc56)c4)cc3)cc21 ; CC1(C)c2ccccc2-c2ccc(-c3ccc(N(c4ccc(-c5cccc6c5C(C)(C)c5ccccc5-6)cc4)c4cccc5c4-c4ccccc4C5(c4ccccc4)c4ccccc4)cc3)cc21 ; CC1(C)c2ccccc2-c2ccc(-c3ccc(N(c4ccc(-c5cccc6c5C(C)(C)c5ccccc5-6)cc4)c4cccc5sc6c7ccccc7ccc6c45)cc3)cc21 ; CC1(C)c2ccccc2-c2ccc(-c3ccc(N(c4ccc(-c5cccc6c5C(C)(C)c5ccccc5-6)cc4)c4ccc(-c5cccc6c5sc5c7ccccc7ccc65)cc4)cc3)cc21]

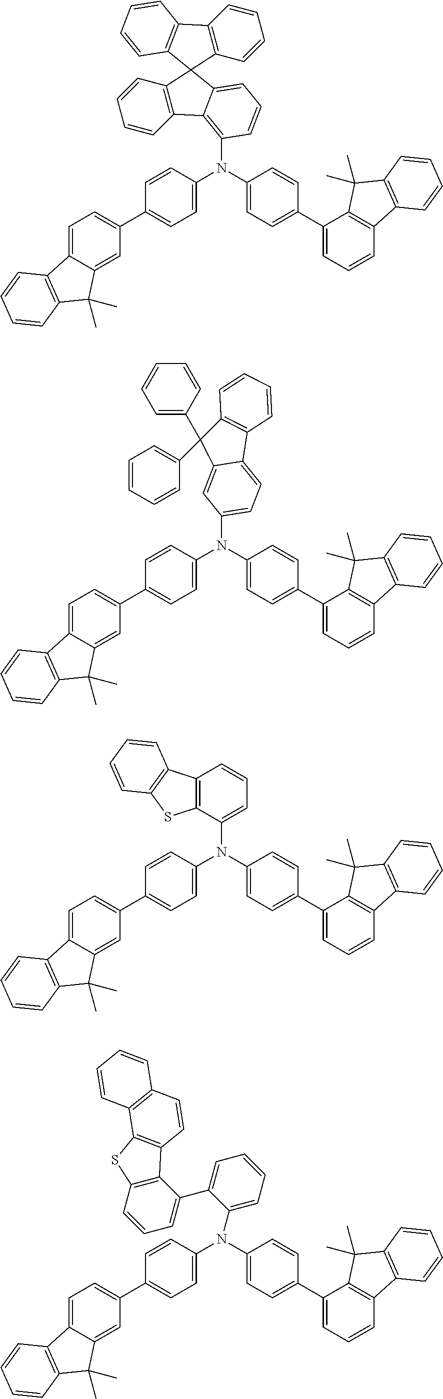 [SMILES: CC1(C)c2ccccc2-c2ccc(-c3ccc(N(c4ccc(-c5cccc6c5C(C)(C)c5ccccc5-6)cc4)c4cccc5c4-c4ccccc4C54c5ccccc5-c5ccccc54)cc3)cc21 ; CC1(C)c2ccccc2-c2ccc(-c3ccc(N(c4ccc(-c5cccc6c5C(C)(C)c5ccccc5-6)cc4)c4ccc5c(c4)C(c4ccccc4)(c4ccccc4)c4ccccc4-5)cc3)cc21 ; CC1(C)c2ccccc2-c2ccc(-c3ccc(N(c4ccc(-c5cccc6c5C(C)(C)c5ccccc5-6)cc4)c4cccc5c4sc4ccccc45)cc3)cc21 ; CC1(C)c2ccccc2-c2ccc(-c3ccc(N(c4ccc(-c5cccc6c5C(C)(C)c5ccccc5-6)cc4)c4ccccc4-c4cccc5sc6c7ccccc7ccc6c45)cc3)cc21]

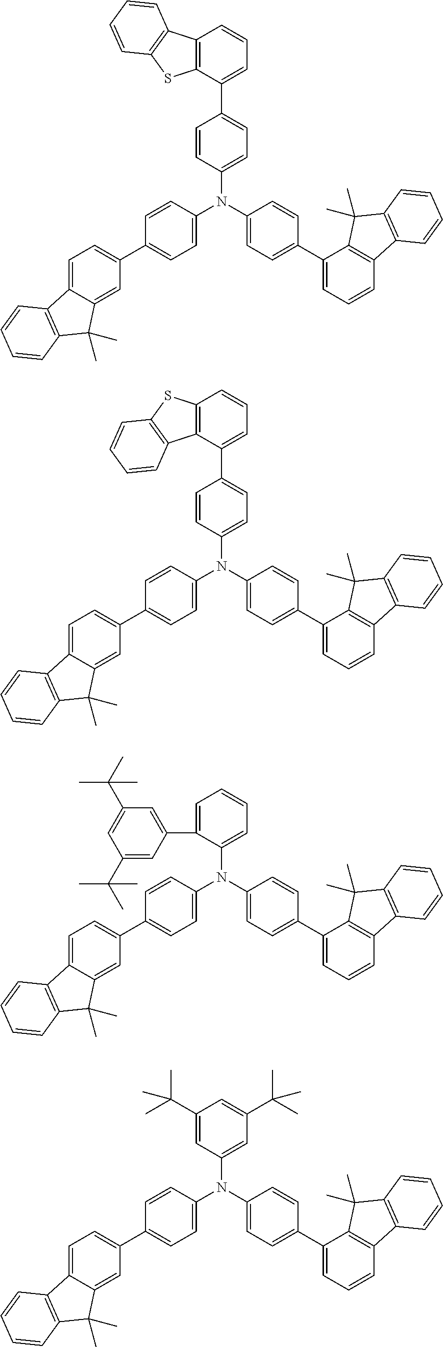

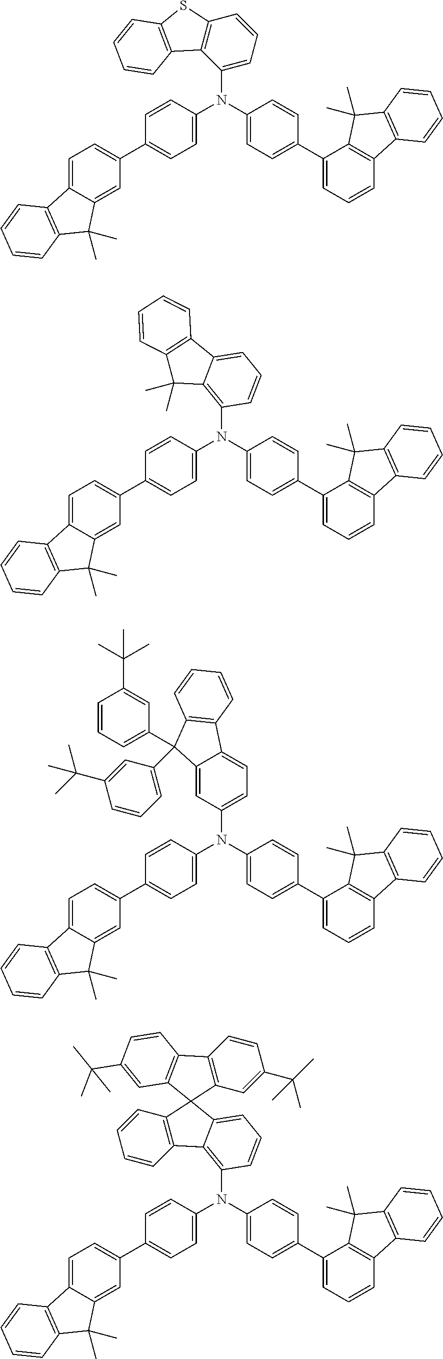 [SMILES: CC1(C)c2ccccc2-c2ccc(-c3ccc(N(c4ccc(-c5cccc6c5C(C)(C)c5ccccc5-6)cc4)c4cccc5sc6ccccc6c45)cc3)cc21 ; CC1(C)c2ccccc2-c2ccc(-c3ccc(N(c4ccc(-c5cccc6c5C(C)(C)c5ccccc5-6)cc4)c4cccc5c4C(C)(C)c4ccccc4-5)cc3)cc21 ; CC(C)(C)c1cccc(C2(c3cccc(C(C)(C)C)c3)c3ccccc3-c3ccc(N(c4ccc(-c5ccc6c(c5)C(C)(C)c5ccccc5-6)cc4)c4ccc(-c5cccc6c5C(C)(C)c5ccccc5-6)cc4)cc32)c1 ; CC(C)(C)c1ccc2c(c1)C1(c3cc(C(C)(C)C)ccc3-2)c2ccccc2-c2c(N(c3ccc(-c4ccc5c(c4)C(C)(C)c4ccccc4-5)cc3)c3ccc(-c4cccc5c4C(C)(C)c4ccccc4-5)cc3)cccc21]

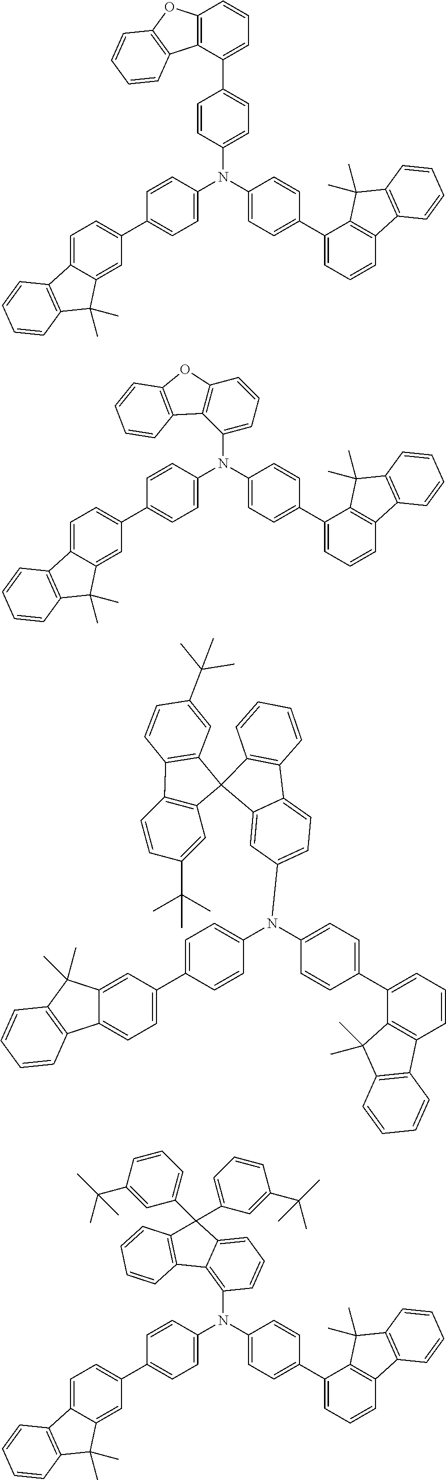 [SMILES: CC1(C)c2ccccc2-c2ccc(-c3ccc(N(c4ccc(-c5cccc6c5C(C)(C)c5ccccc5-6)cc4)c4ccc(-c5cccc6oc7ccccc7c56)cc4)cc3)cc21 ; CC1(C)c2ccccc2-c2ccc(-c3ccc(N(c4ccc(-c5cccc6c5C(C)(C)c5ccccc5-6)cc4)c4cccc5oc6ccccc6c45)cc3)cc21 ; CC(C)(C)c1ccc2c(c1)C1(c3ccccc3-c3ccc(N(c4ccc(-c5ccc6c(c5)C(C)(C)c5ccccc5-6)cc4)c4ccc(-c5cccc6c5C(C)(C)c5ccccc5-6)cc4)cc31)c1cc(C(C)(C)C)ccc1-2 ; CC(C)(C)c1cccc(C2(c3cccc(C(C)(C)C)c3)c3ccccc3-c3c(N(c4ccc(-c5ccc6c(c5)C(C)(C)c5ccccc5-6)cc4)c4ccc(-c5cccc6c5C(C)(C)c5ccccc5-6)cc4)cccc32)c1]

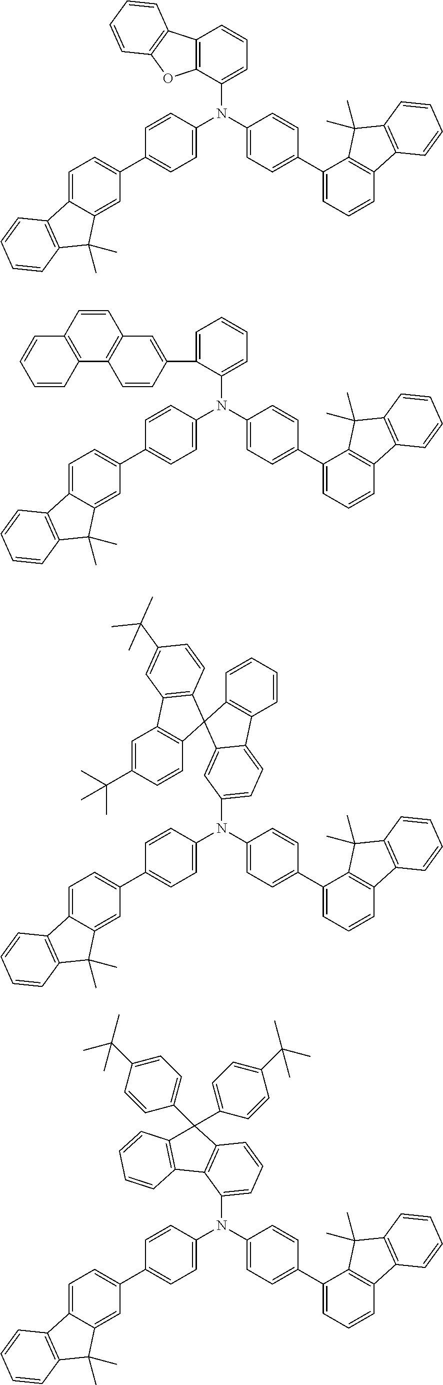

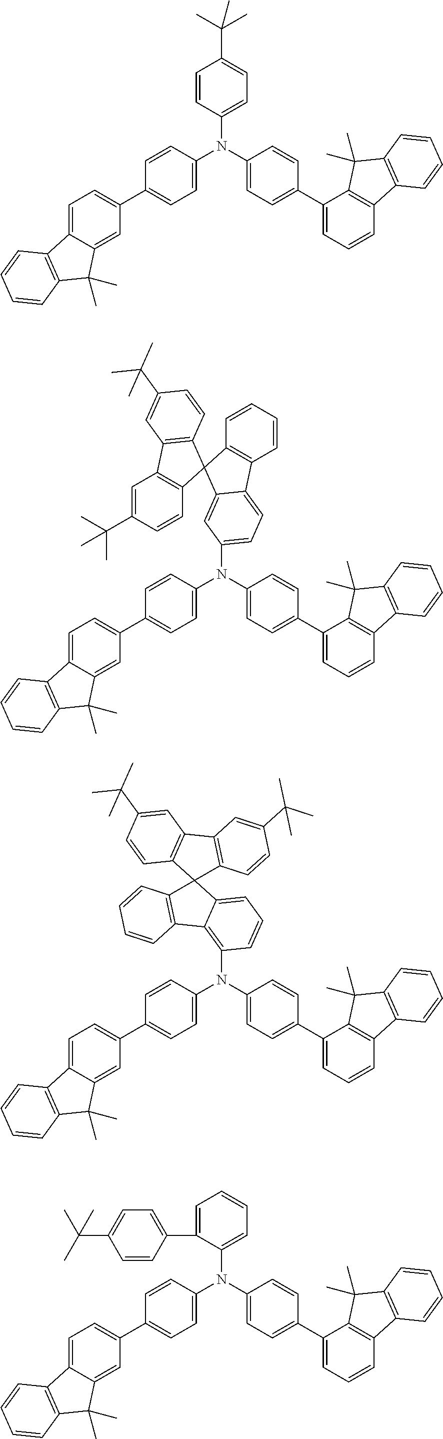 [SMILES: CC(C)(C)c1ccc(N(c2ccc(-c3ccc4c(c3)C(C)(C)c3ccccc3-4)cc2)c2ccc(-c3cccc4c3C(C)(C)c3ccccc3-4)cc2)cc1 ; CC(C)(C)c1ccc2c(c1)-c1cc(C(C)(C)C)ccc1C21c2ccccc2-c2ccc(N(c3ccc(-c4ccc5c(c4)C(C)(C)c4ccccc4-5)cc3)c3ccc(-c4cccc5c4C(C)(C)c4ccccc4-5)cc3)cc21 ; CC(C)(C)c1ccc2c(c1)-c1cc(C(C)(C)C)ccc1C21c2ccccc2-c2c(N(c3ccc(-c4ccc5c(c4)C(C)(C)c4ccccc4-5)cc3)c3ccc(-c4cccc5c4C(C)(C)c4ccccc4-5)cc3)cccc21 ; CC(C)(C)c1ccc(-c2ccccc2N(c2ccc(-c3ccc4c(c3)C(C)(C)c3ccccc3-4)cc2)c2ccc(-c3cccc4c3C(C)(C)c3ccccc3-4)cc2)cc1]

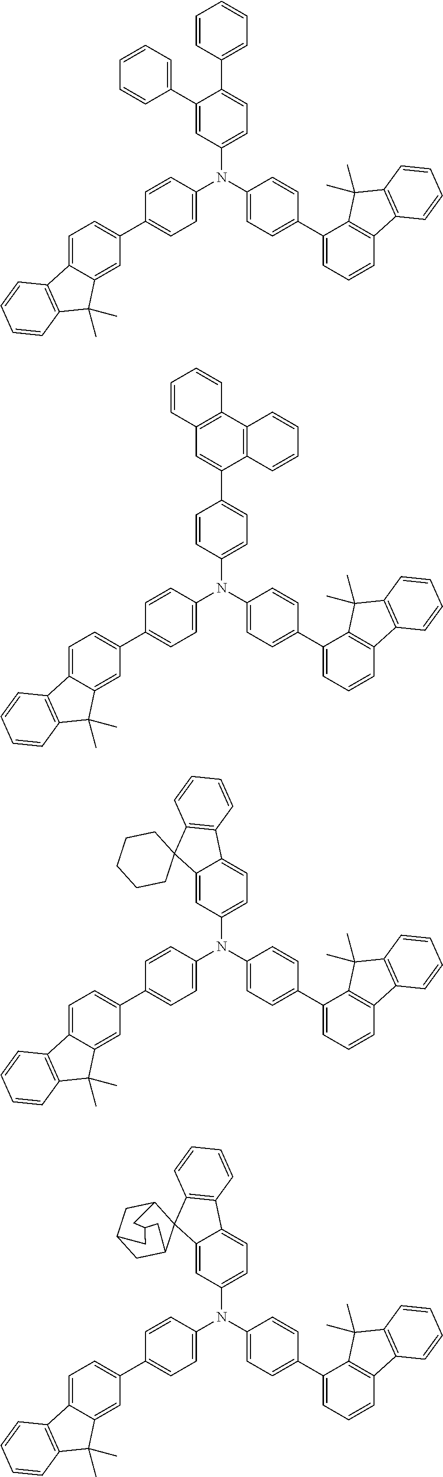 [SMILES: CC1(C)c2ccccc2-c2ccc(-c3ccc(N(c4ccc(-c5cccc6c5C(C)(C)c5ccccc5-6)cc4)c4ccc(-c5ccccc5)c(-c5ccccc5)c4)cc3)cc21 ; CC1(C)c2ccccc2-c2ccc(-c3ccc(N(c4ccc(-c5cccc6c5C(C)(C)c5ccccc5-6)cc4)c4ccc(-c5cc6ccccc6c6ccccc56)cc4)cc3)cc21 ; CC1(C)c2ccccc2-c2ccc(-c3ccc(N(c4ccc(-c5cccc6c5C(C)(C)c5ccccc5-6)cc4)c4ccc5c(c4)C4(CCCCC4)c4ccccc4-5)cc3)cc21 ; CC1(C)c2ccccc2-c2ccc(-c3ccc(N(c4ccc(-c5cccc6c5C(C)(C)c5ccccc5-6)cc4)c4ccc5c(c4)C4(c6ccccc6-5)C5CC6CC(C5)CC4C6)cc3)cc21]

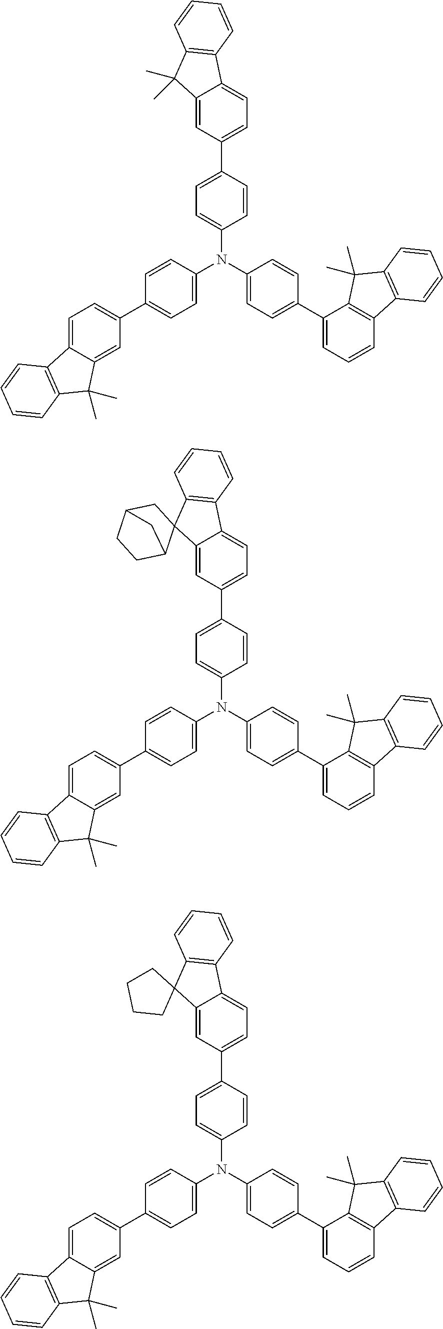 [SMILES: CC1(C)c2ccccc2-c2ccc(-c3ccc(N(c4ccc(-c5ccc6c(c5)C(C)(C)c5ccccc5-6)cc4)c4ccc(-c5cccc6c5C(C)(C)c5ccccc5-6)cc4)cc3)cc21 ; CC1(C)c2ccccc2-c2ccc(-c3ccc(N(c4ccc(-c5ccc6c(c5)C5(CC7CCC5C7)c5ccccc5-6)cc4)c4ccc(-c5cccc6c5C(C)(C)c5ccccc5-6)cc4)cc3)cc21 ; CC1(C)c2ccccc2-c2ccc(-c3ccc(N(c4ccc(-c5ccc6c(c5)C5(CCCC5)c5ccccc5-6)cc4)c4ccc(-c5cccc6c5C(C)(C)c5ccccc5-6)cc4)cc3)cc21]

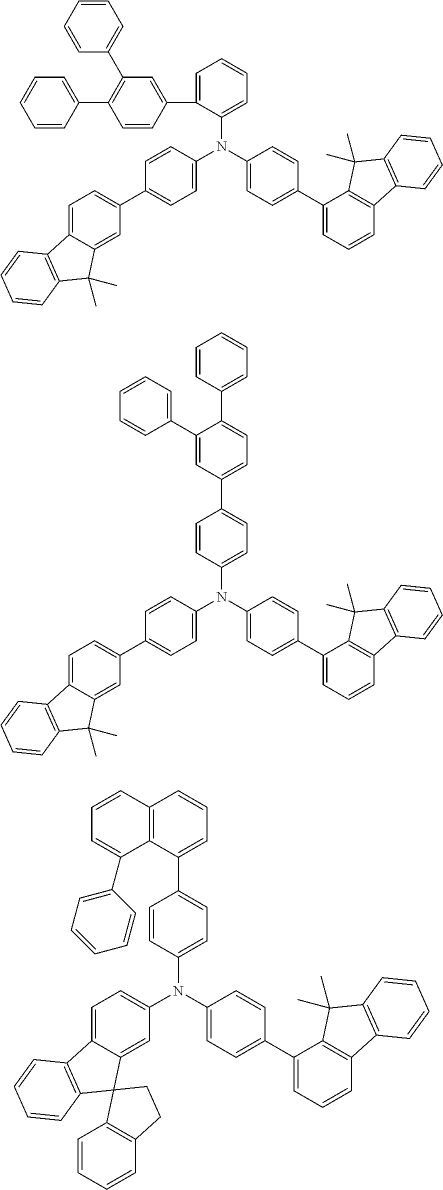 [SMILES: CC1(C)c2ccccc2-c2ccc(-c3ccc(N(c4ccc(-c5cccc6c5C(C)(C)c5ccccc5-6)cc4)c4ccccc4-c4ccc(-c5ccccc5)c(-c5ccccc5)c4)cc3)cc21 ; CC1(C)c2ccccc2-c2ccc(-c3ccc(N(c4ccc(-c5ccc(-c6ccccc6)c(-c6ccccc6)c5)cc4)c4ccc(-c5cccc6c5C(C)(C)c5ccccc5-6)cc4)cc3)cc21 ; CC1(C)c2ccccc2-c2cccc(-c3ccc(N(c4ccc(-c5cccc6cccc(-c7ccccc7)c56)cc4)c4ccc5c(c4)C4(CCc6ccccc64)c4ccccc4-5)cc3)c21]

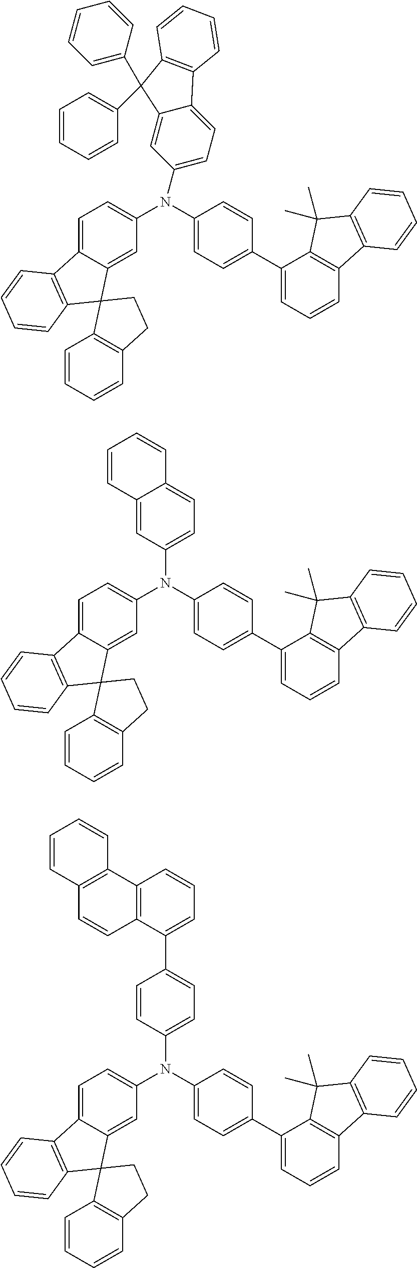

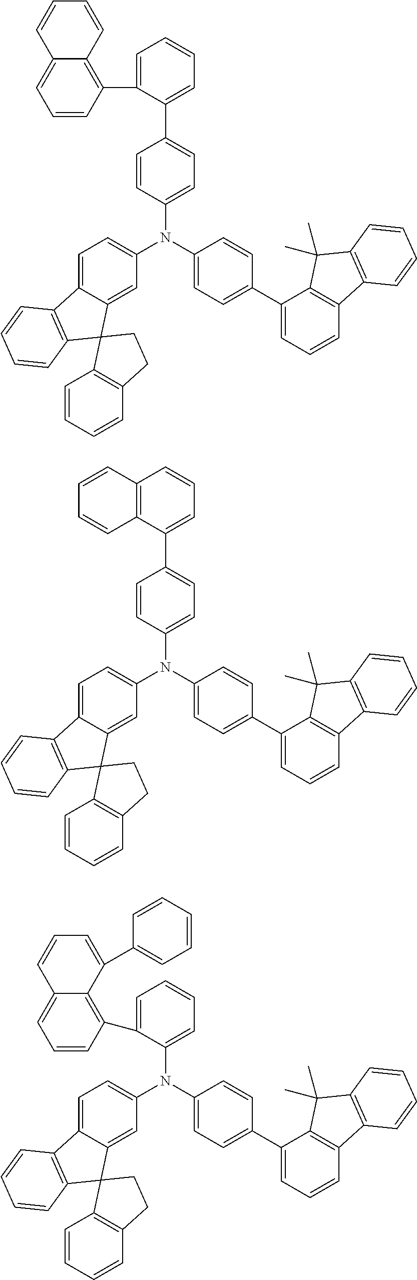

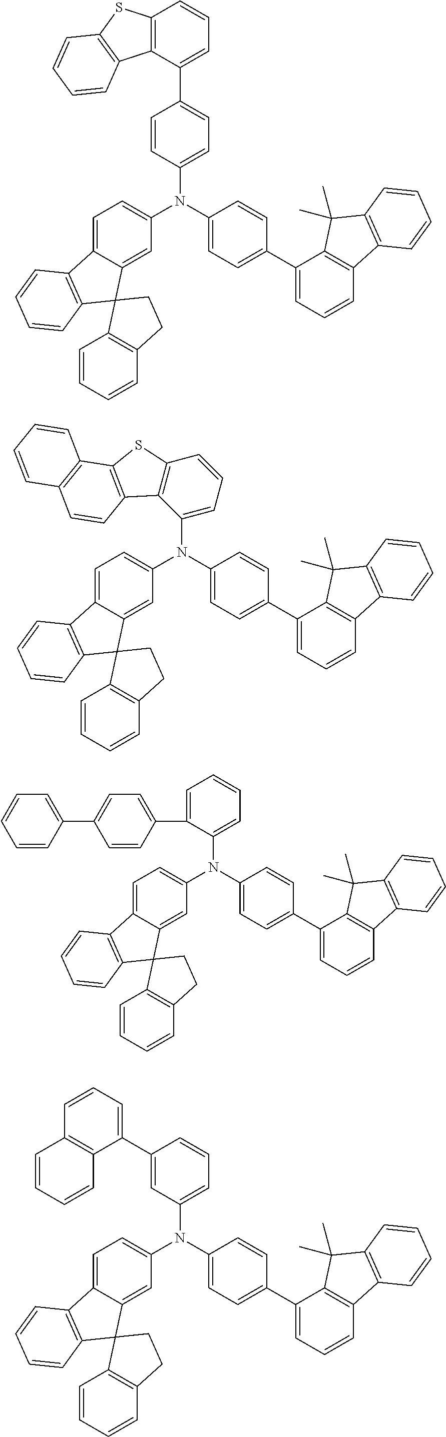

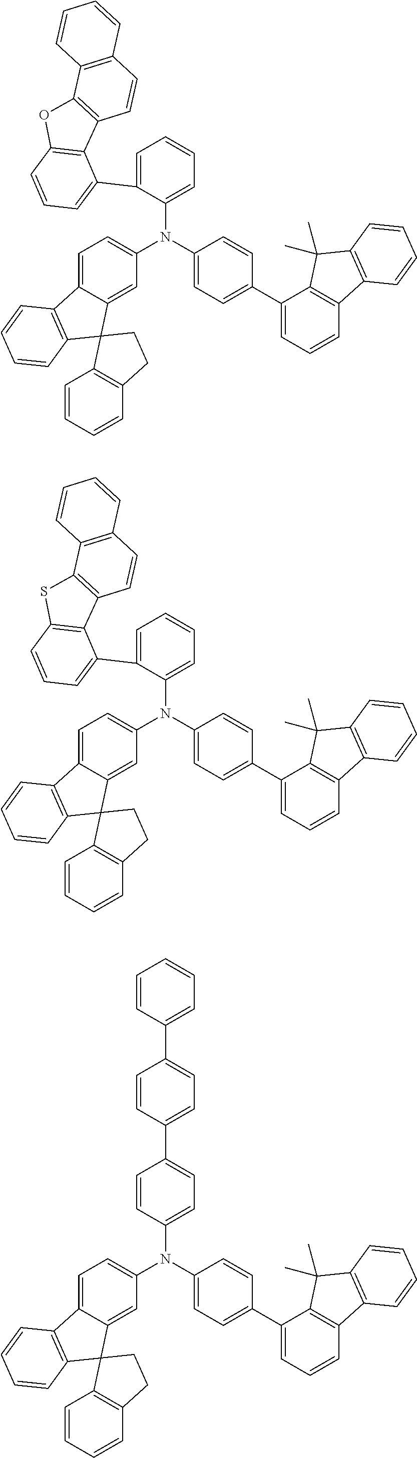

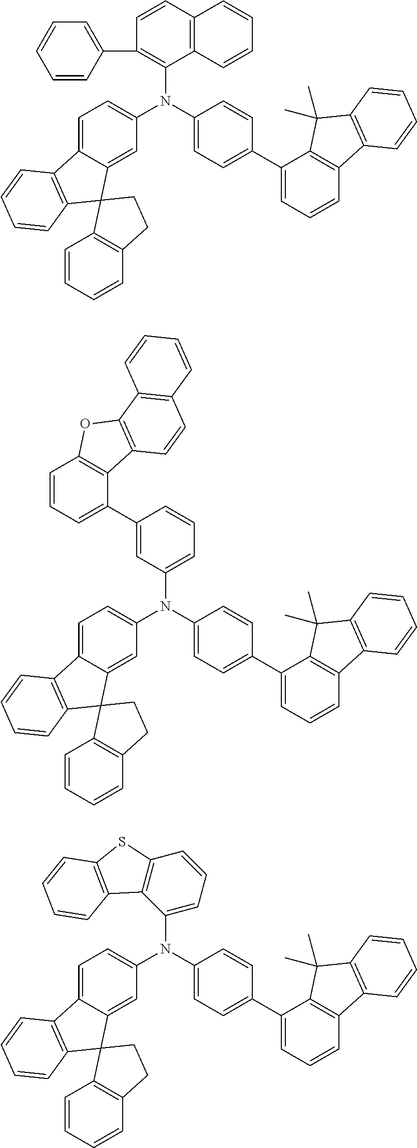

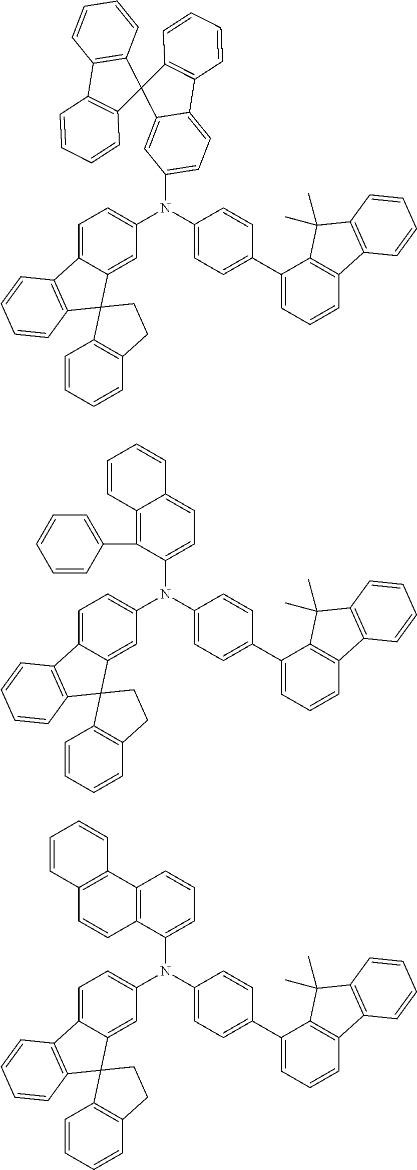

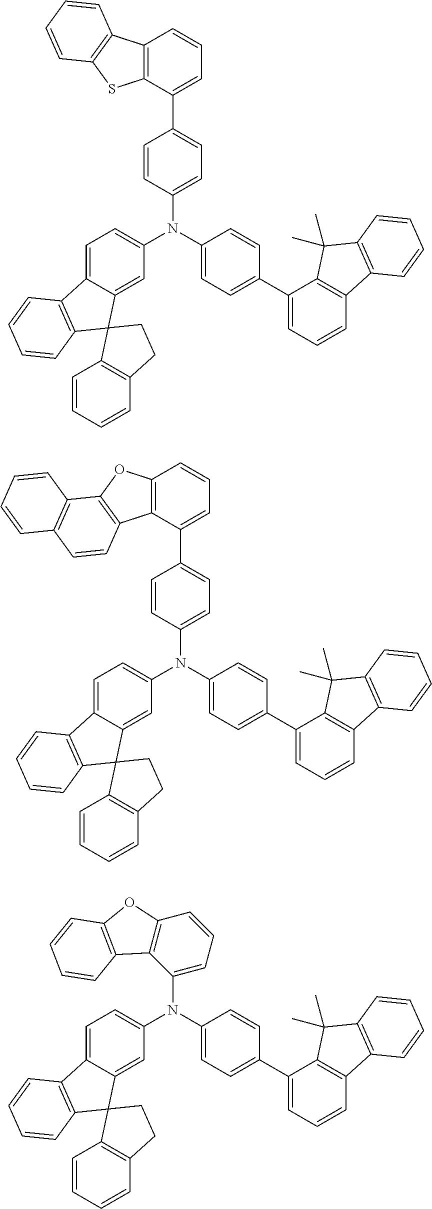

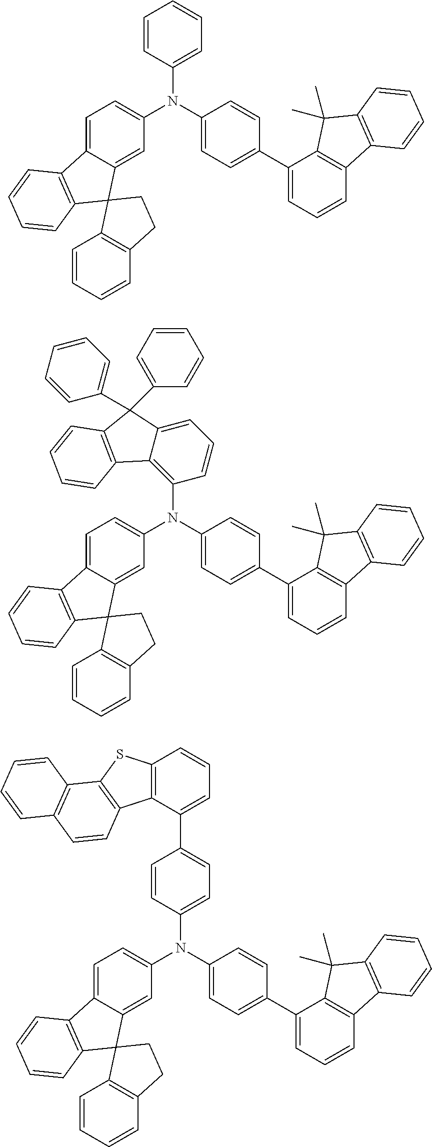

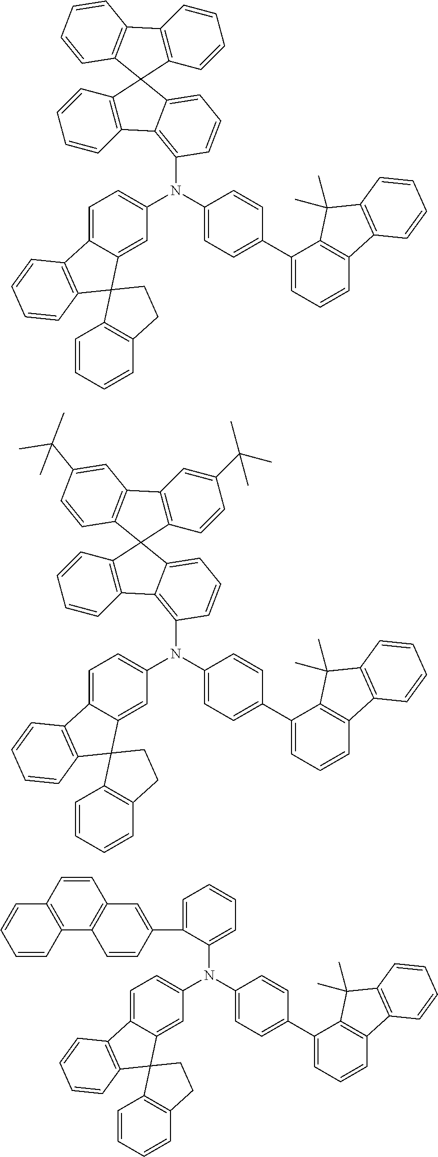

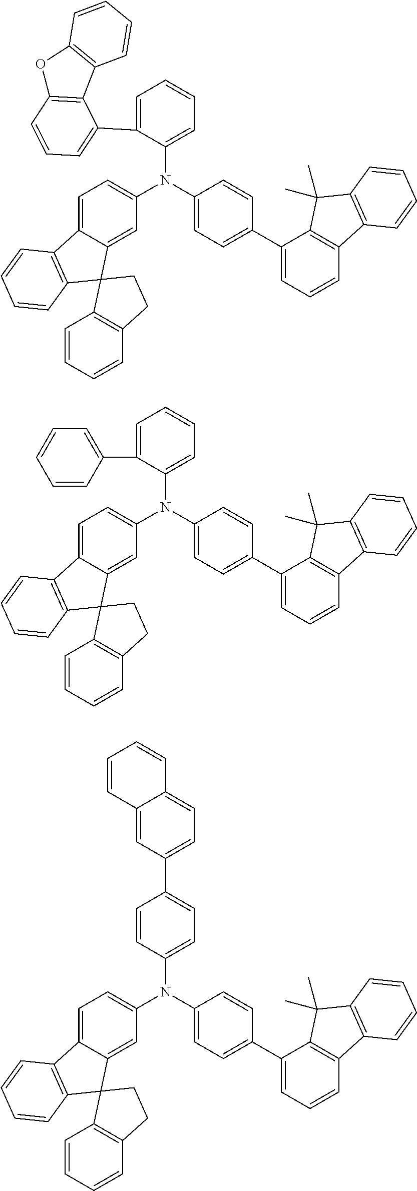 [SMILES: CC1(C)c2ccccc2-c2cccc(-c3ccc(N(c4ccc5c(c4)C4(CCc6ccccc64)c4ccccc4-5)c4ccccc4-c4cccc5oc6ccccc6c45)cc3)c21 ; CC1(C)c2ccccc2-c2cccc(-c3ccc(N(c4ccc5c(c4)C4(CCc6ccccc64)c4ccccc4-5)c4ccccc4-c4ccccc4)cc3)c21 ; CC1(C)c2ccccc2-c2cccc(-c3ccc(N(c4ccc(-c5ccc6ccccc6c5)cc4)c4ccc5c(c4)C4(CCc6ccccc64)c4ccccc4-5)cc3)c21]

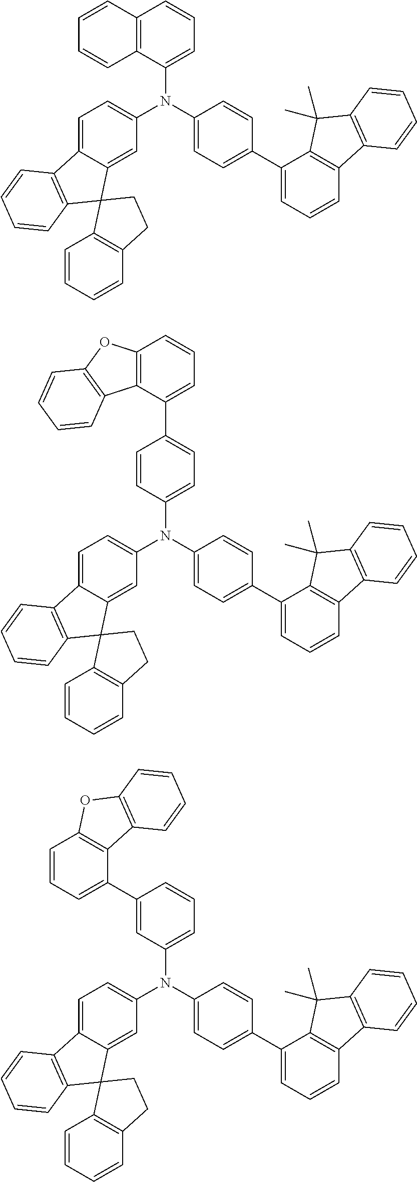

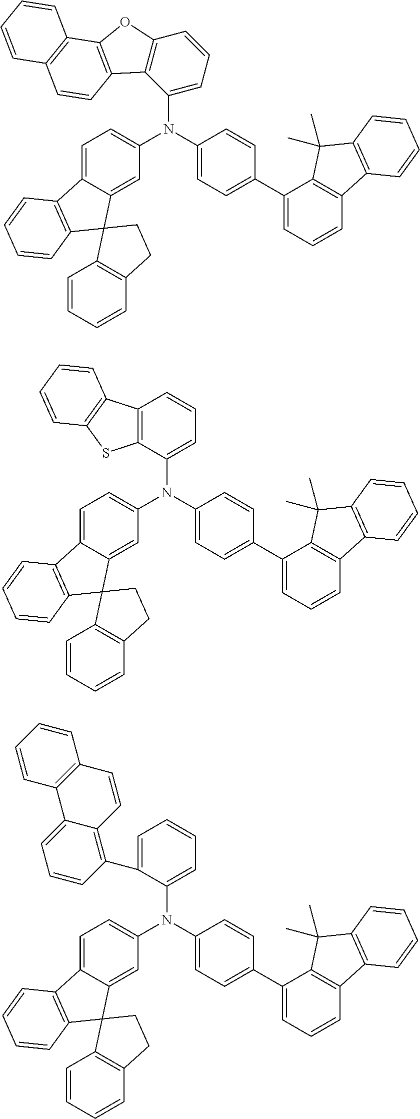

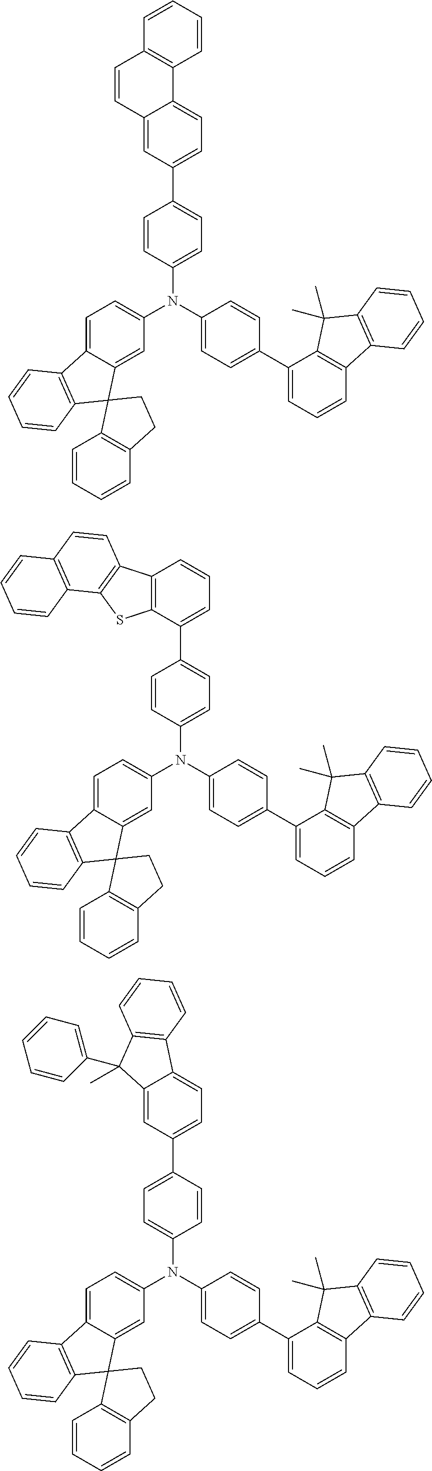

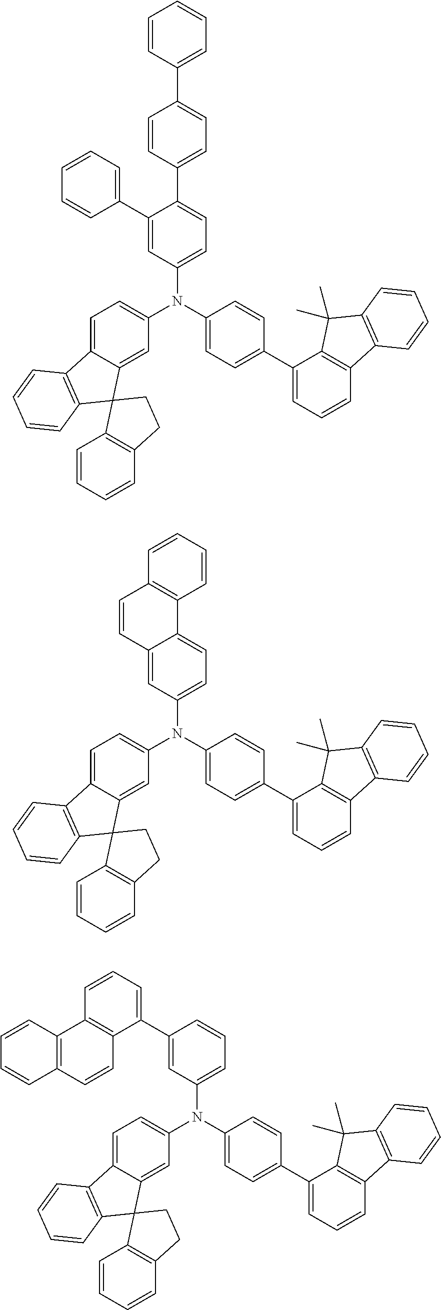

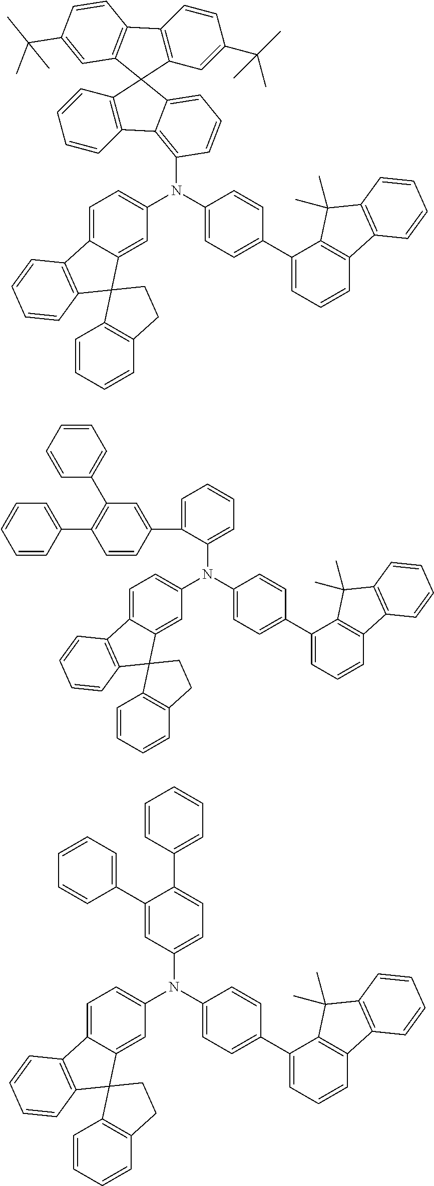

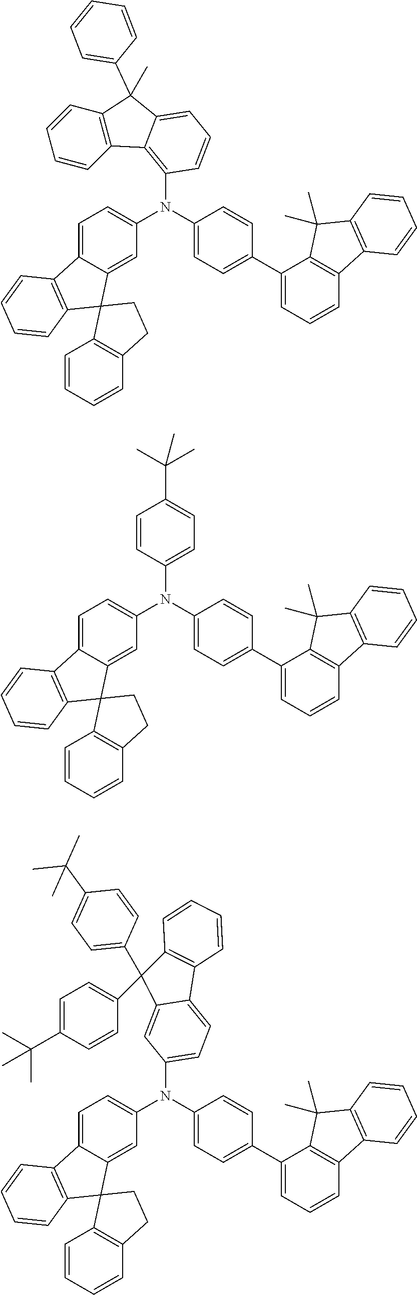

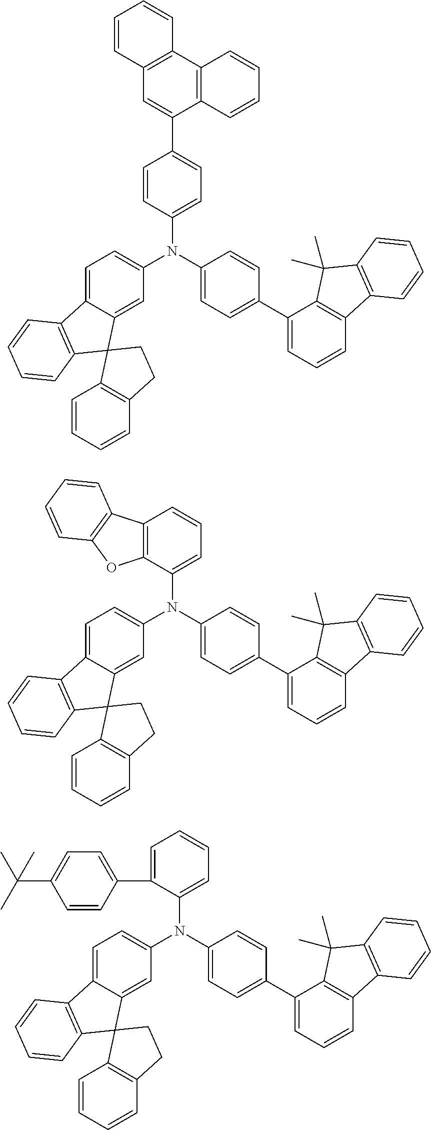

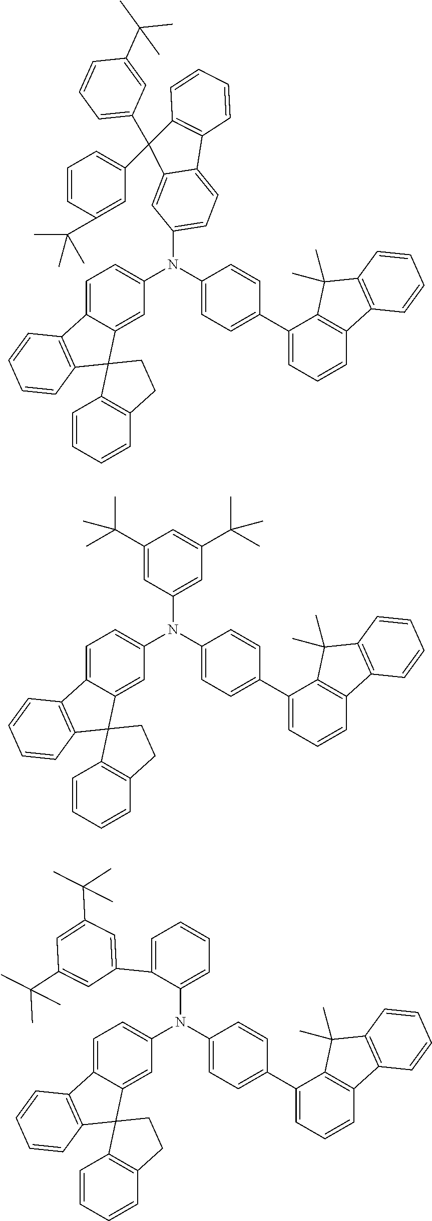

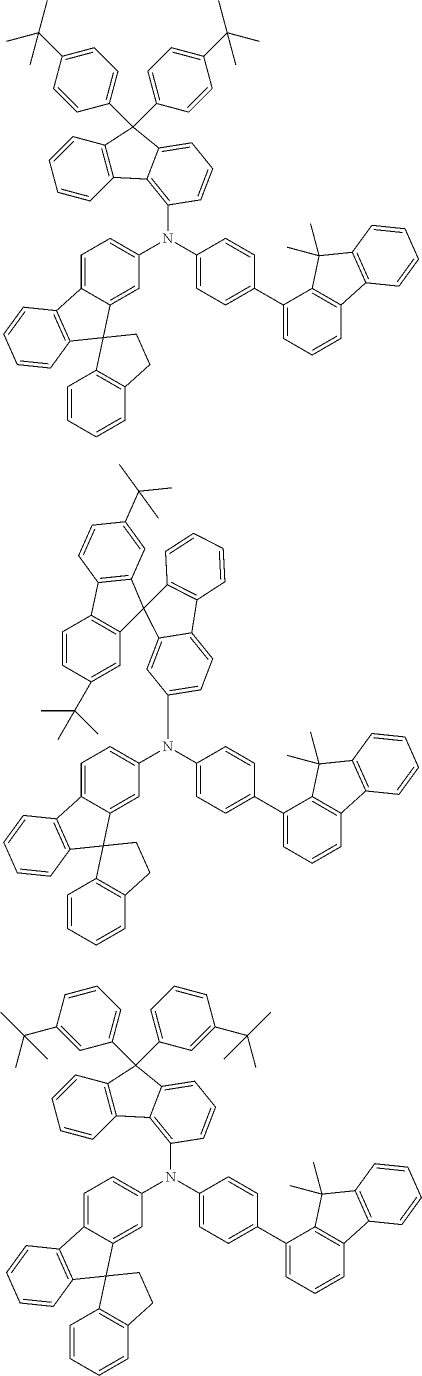

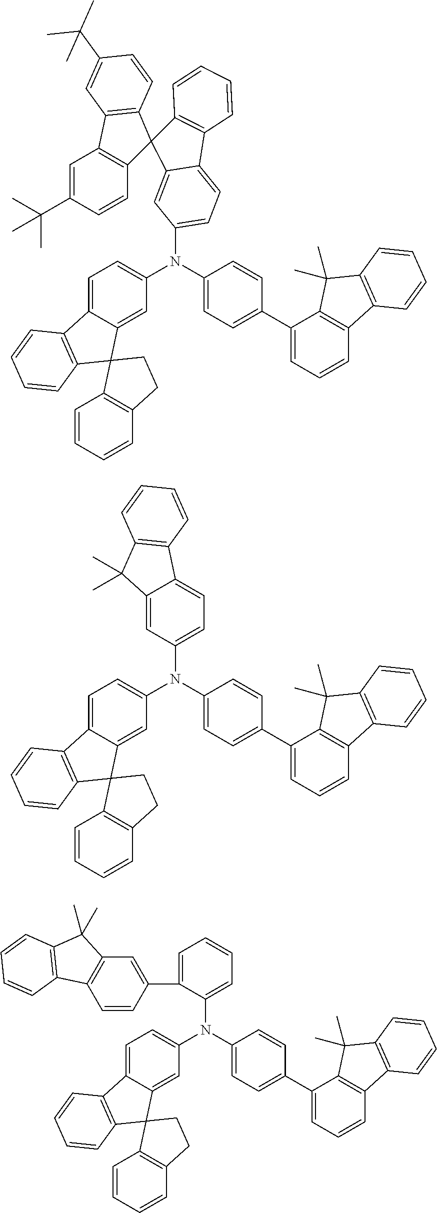

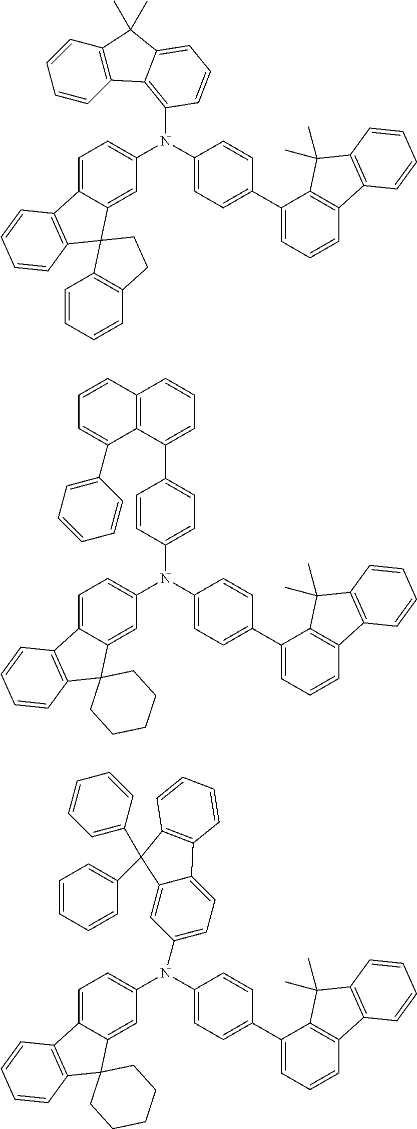

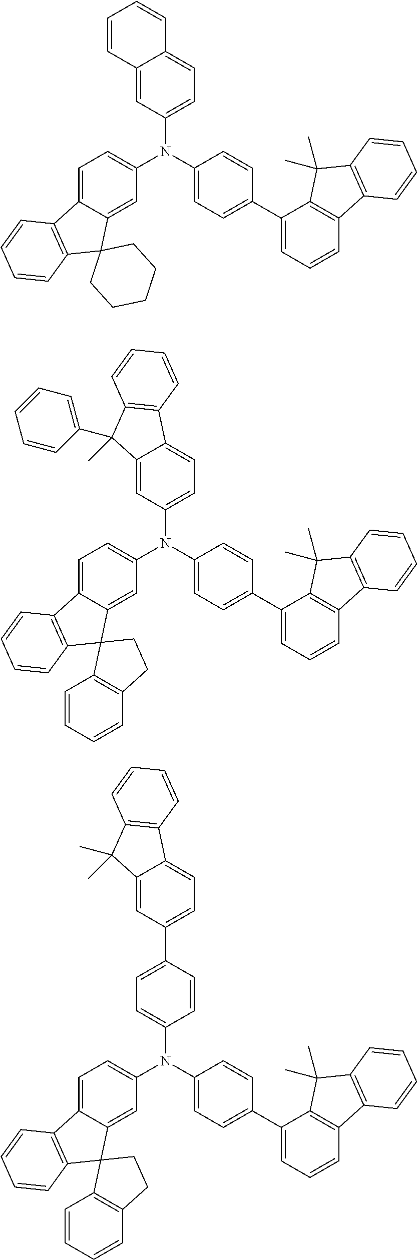

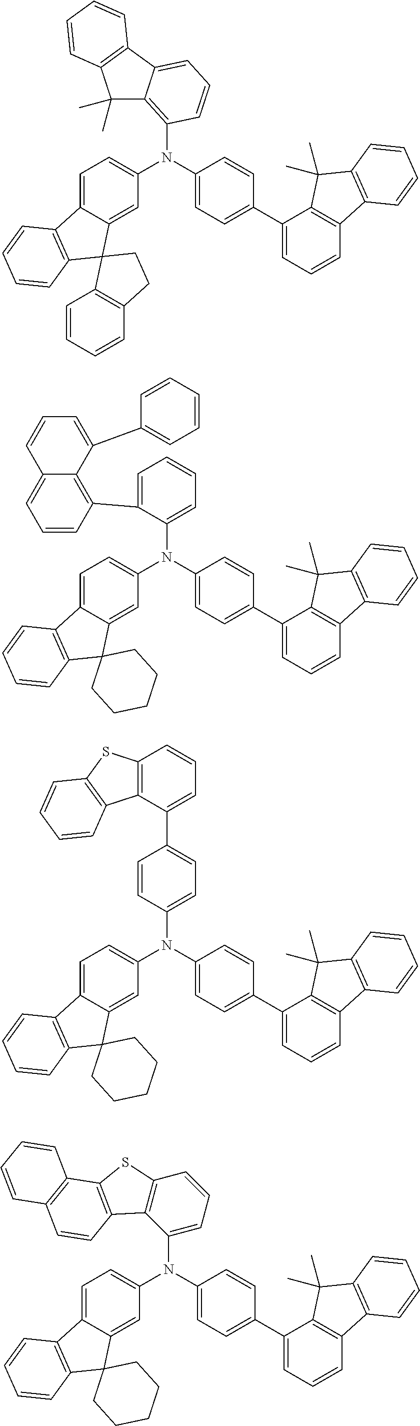

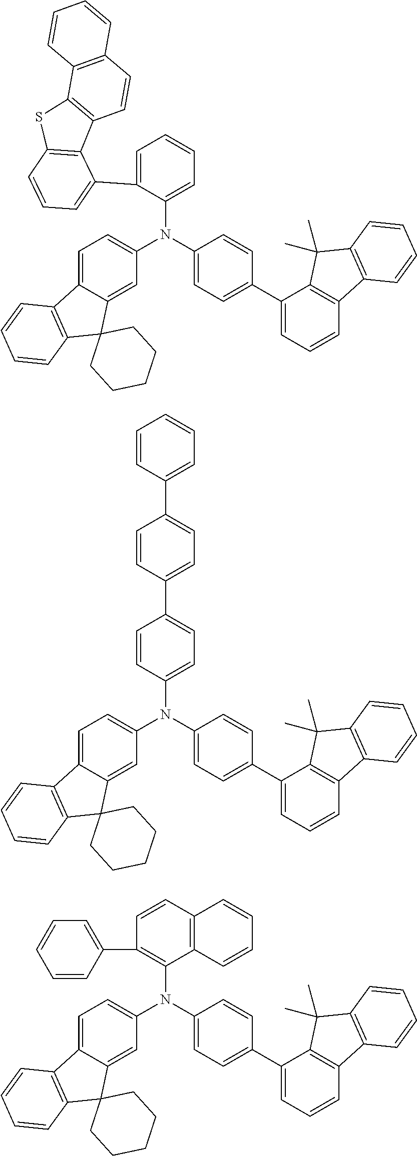

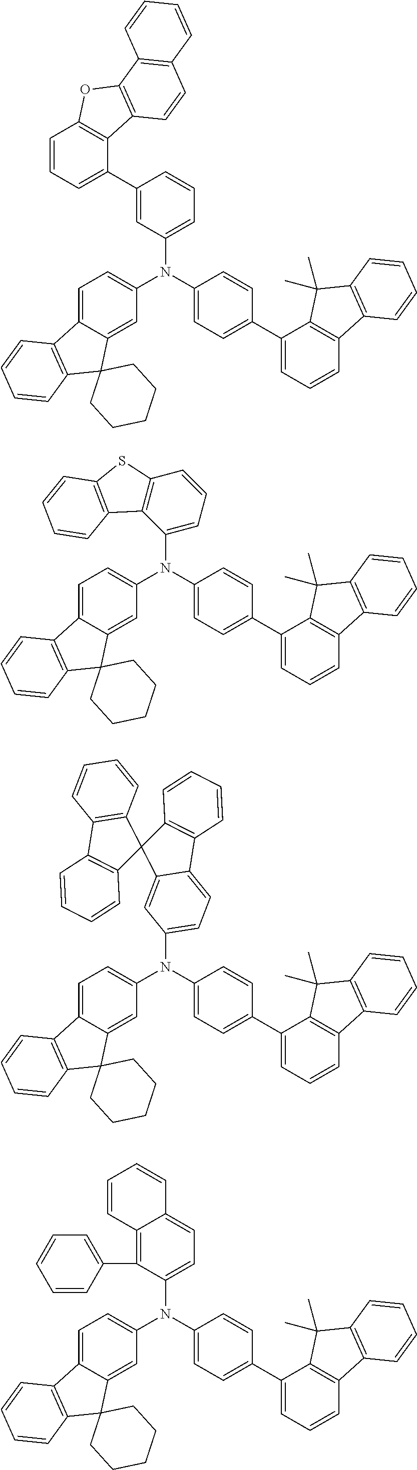

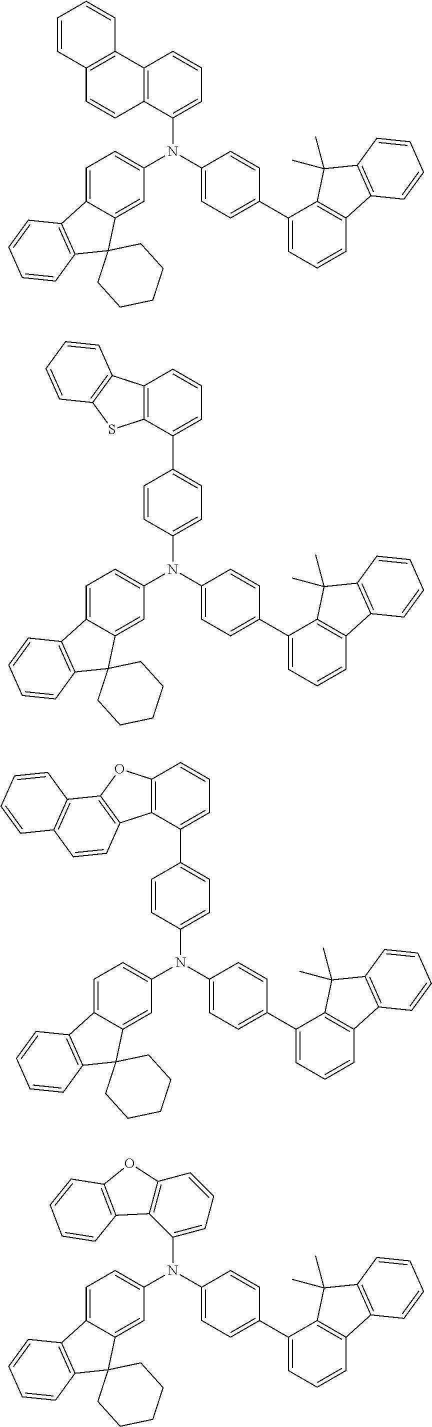

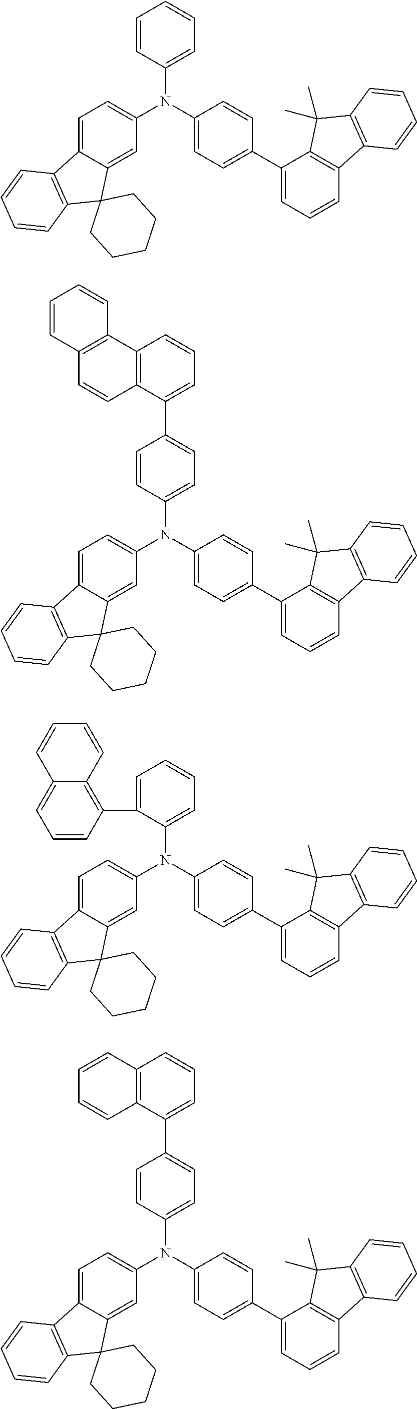

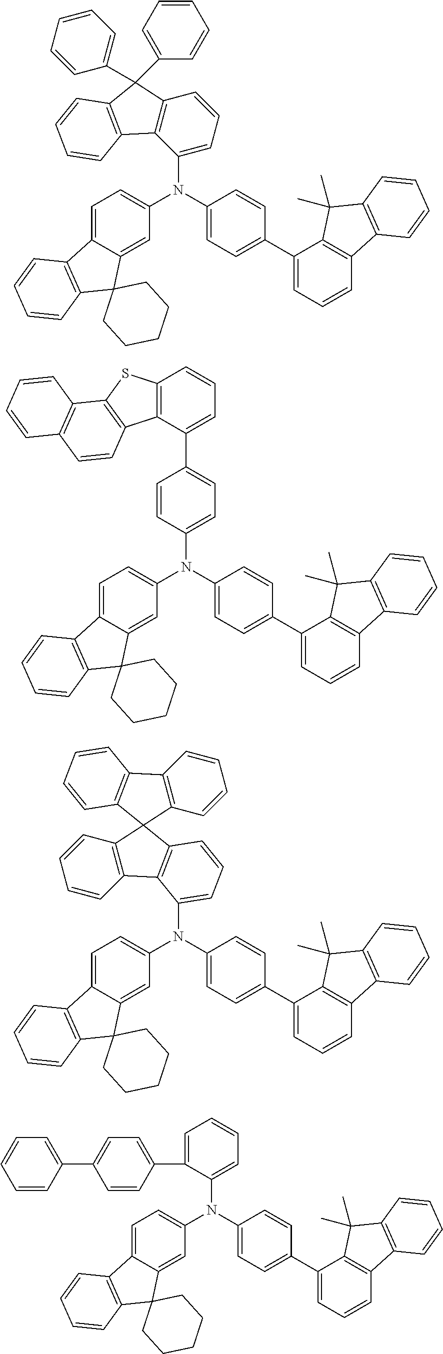

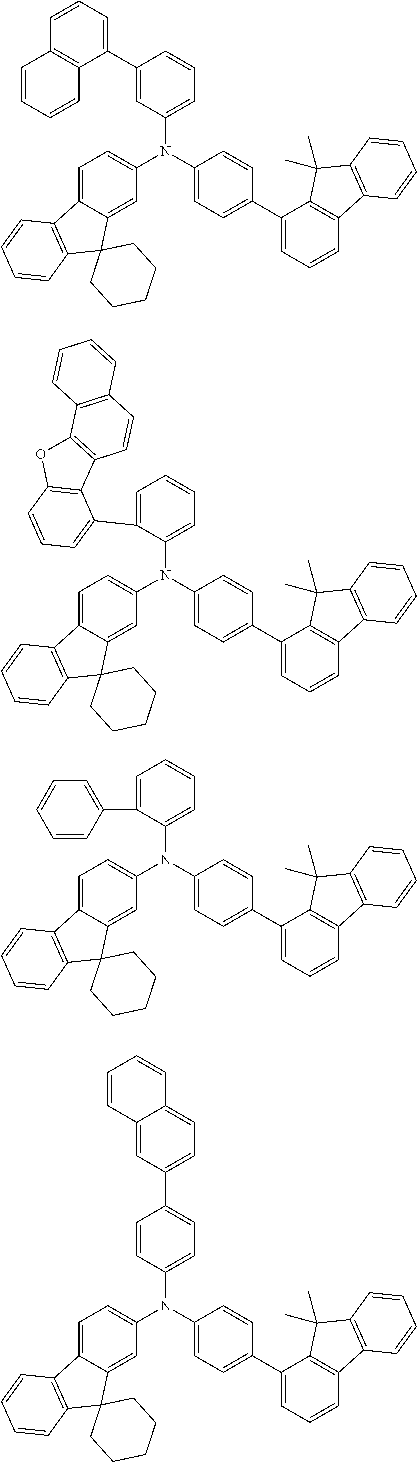

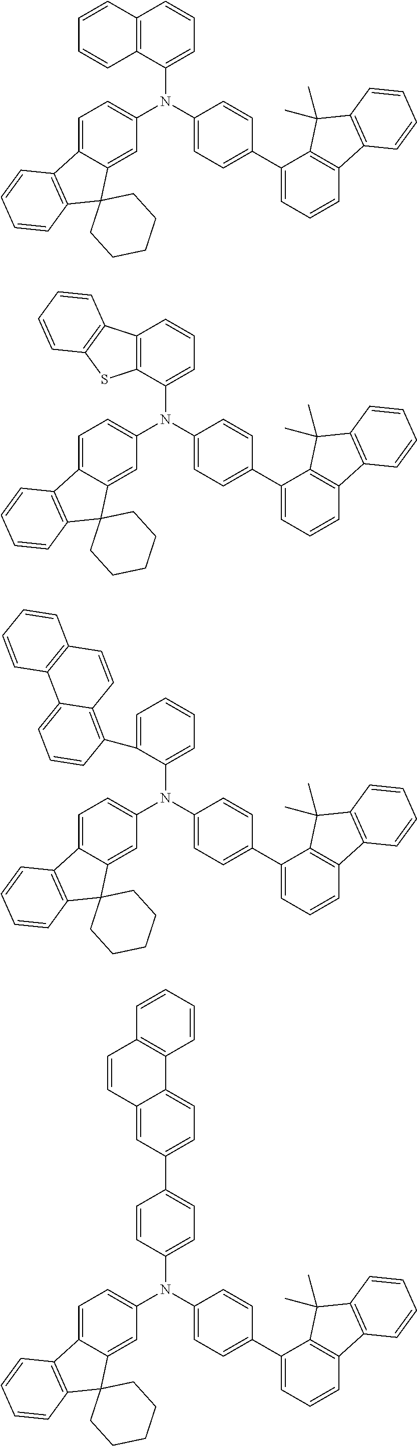

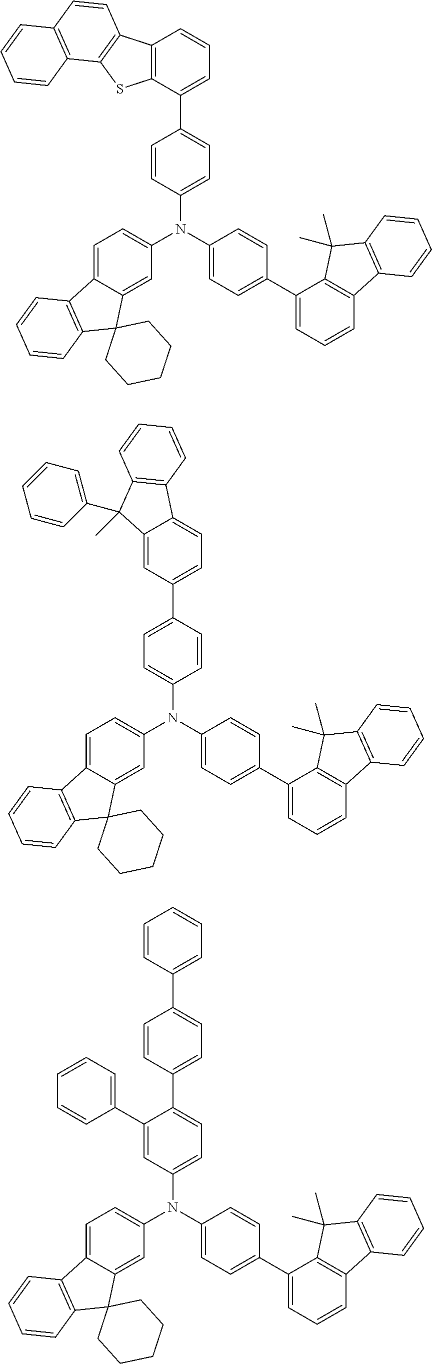

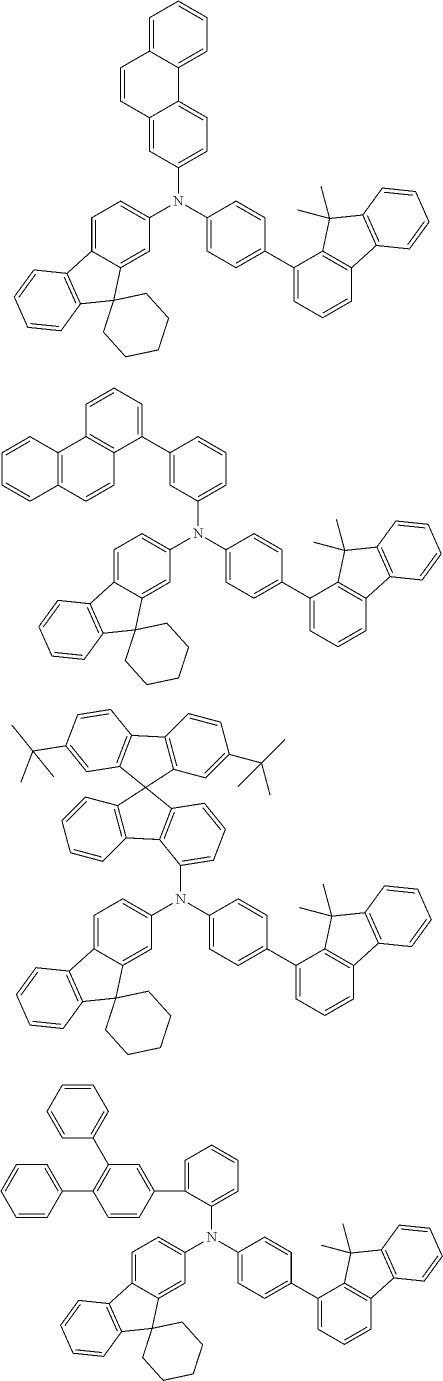

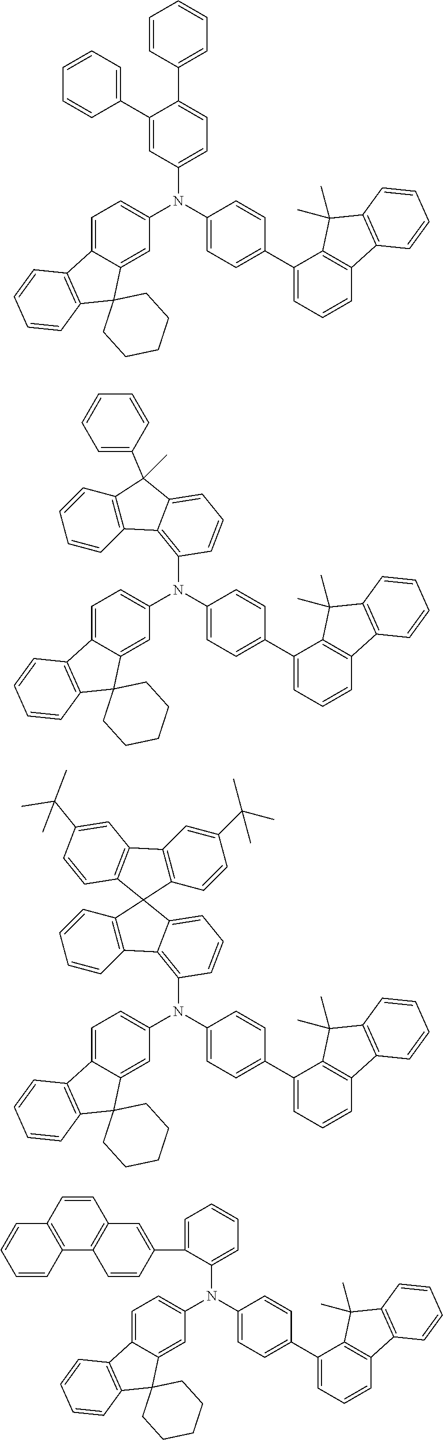

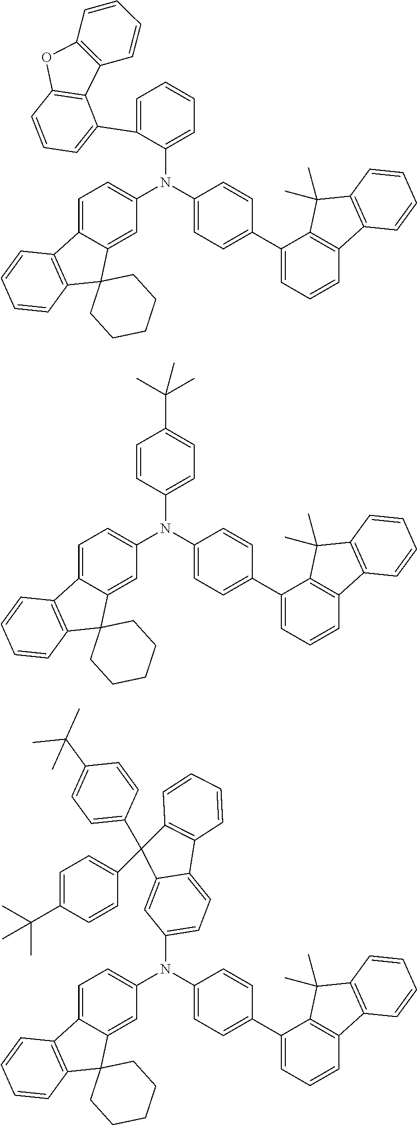

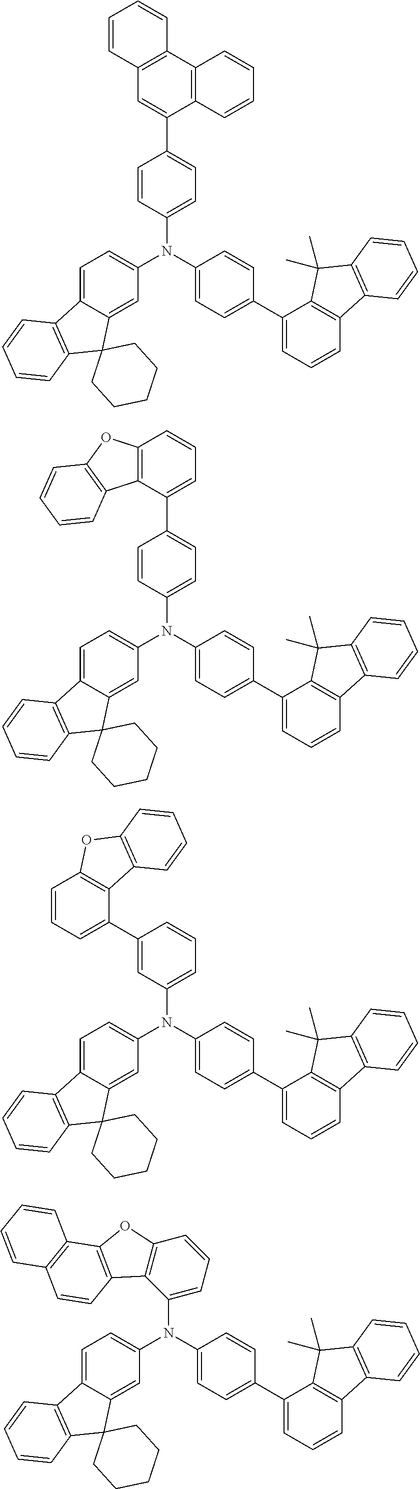

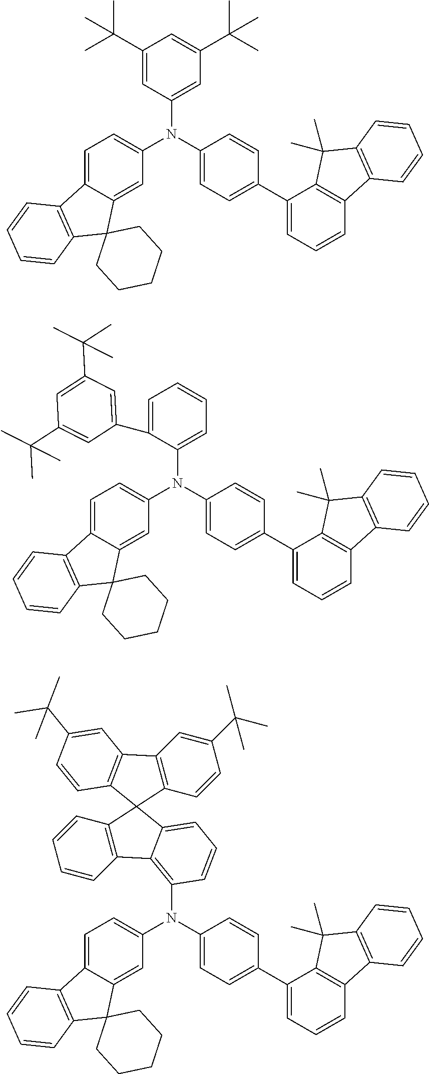

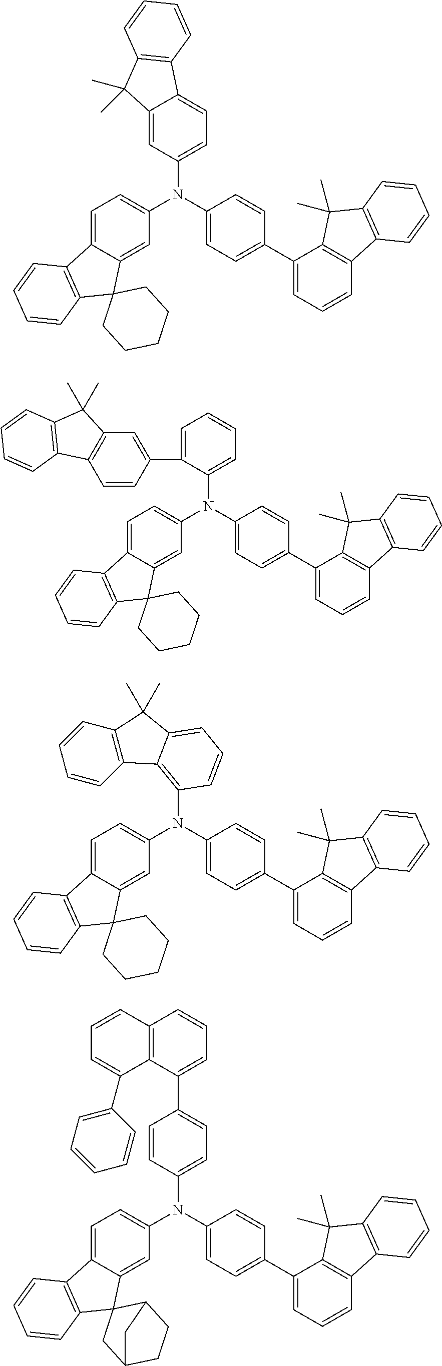

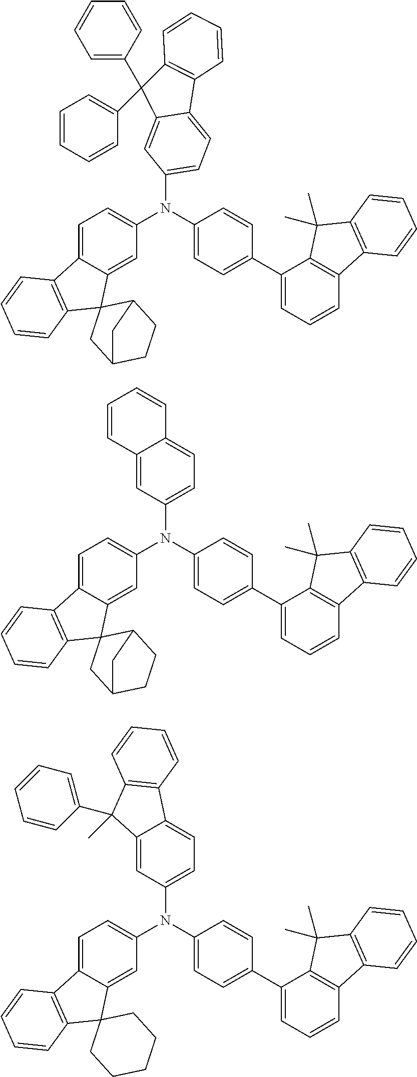

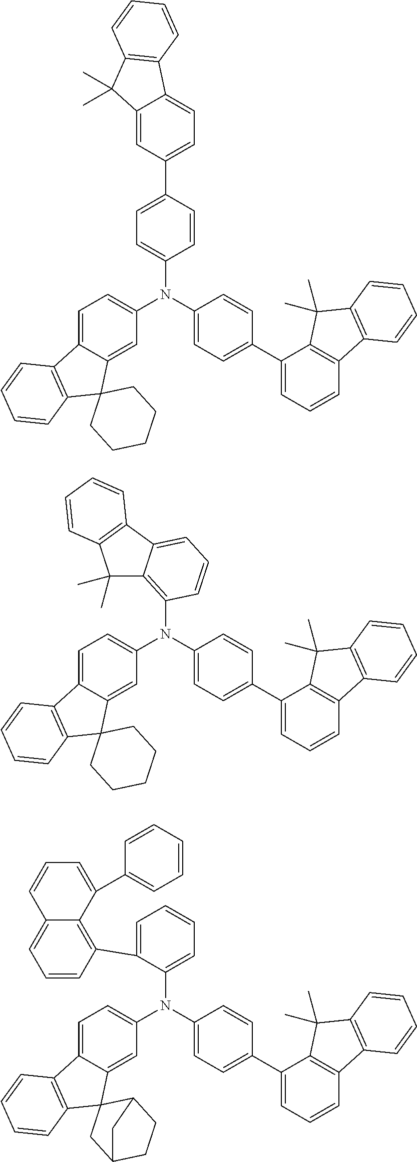

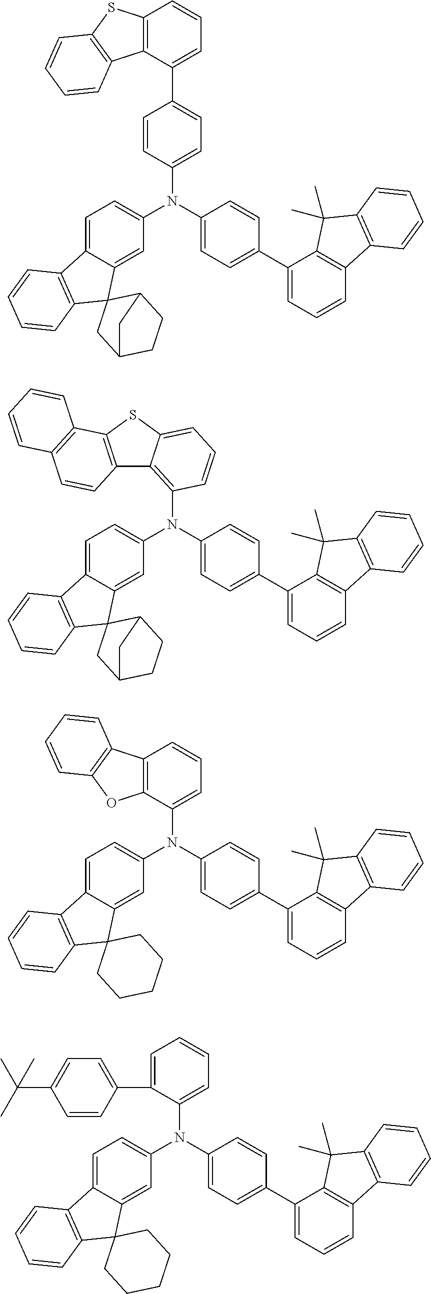

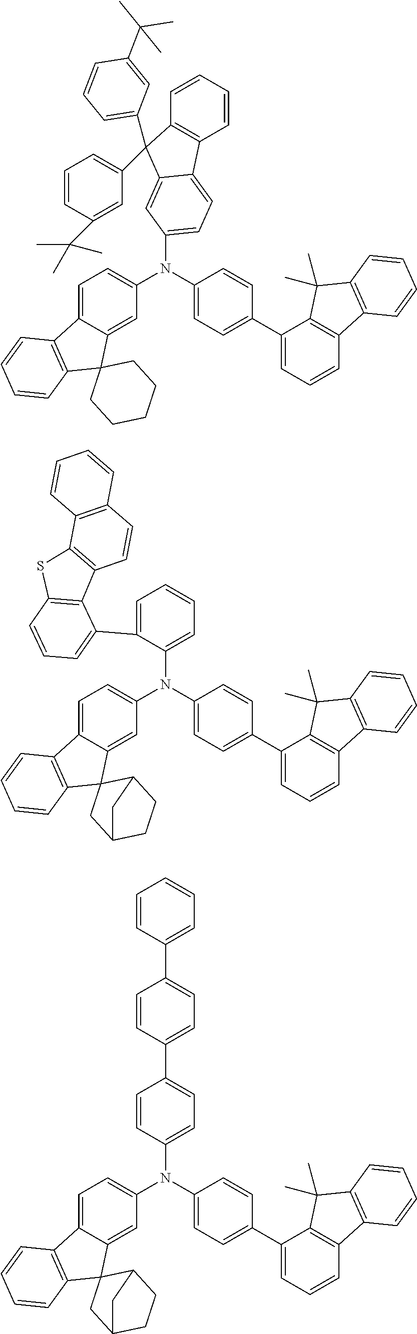

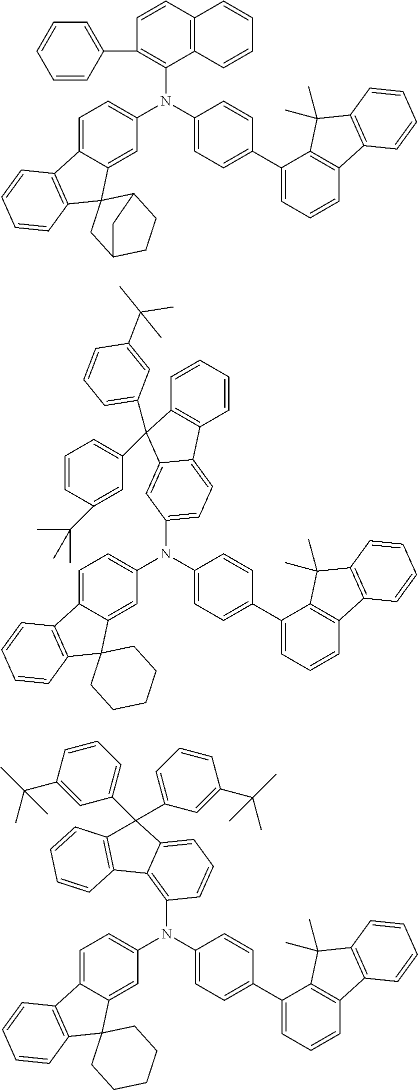

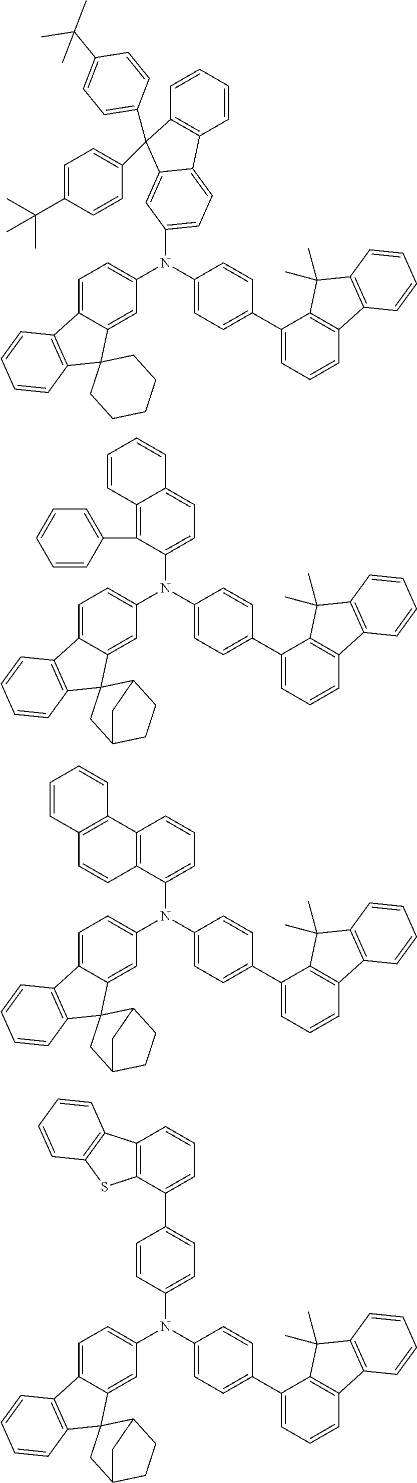

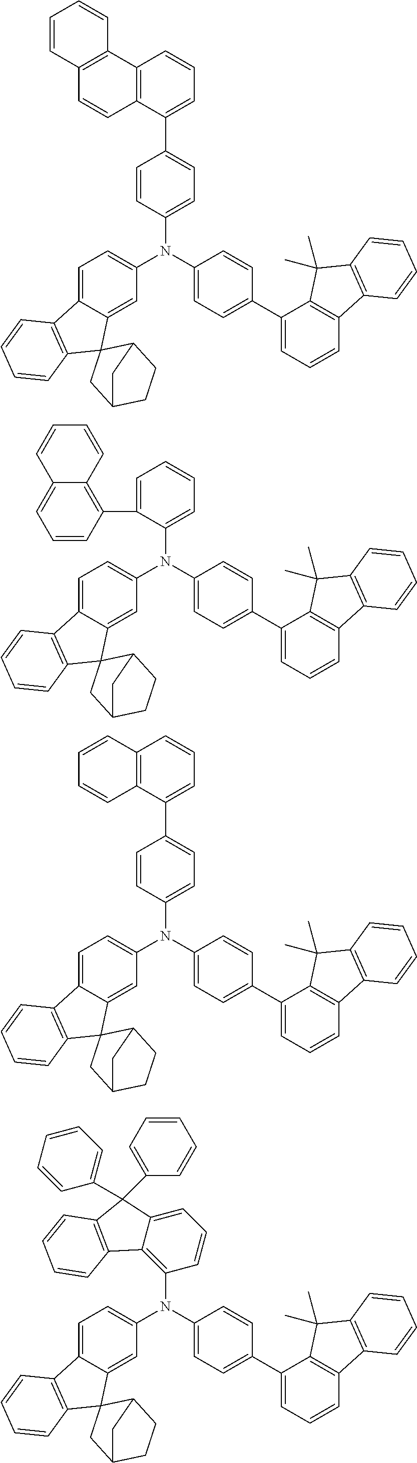

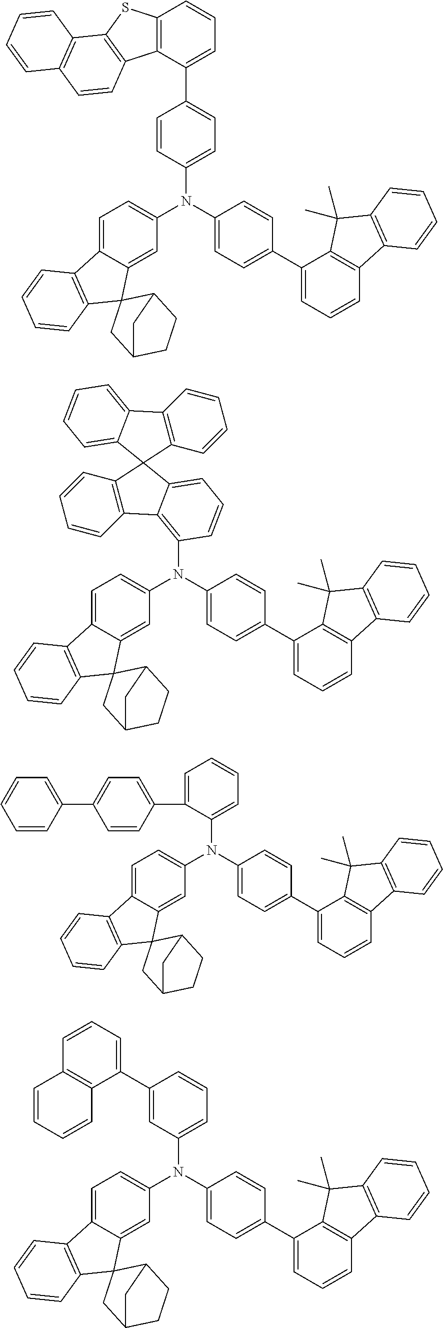

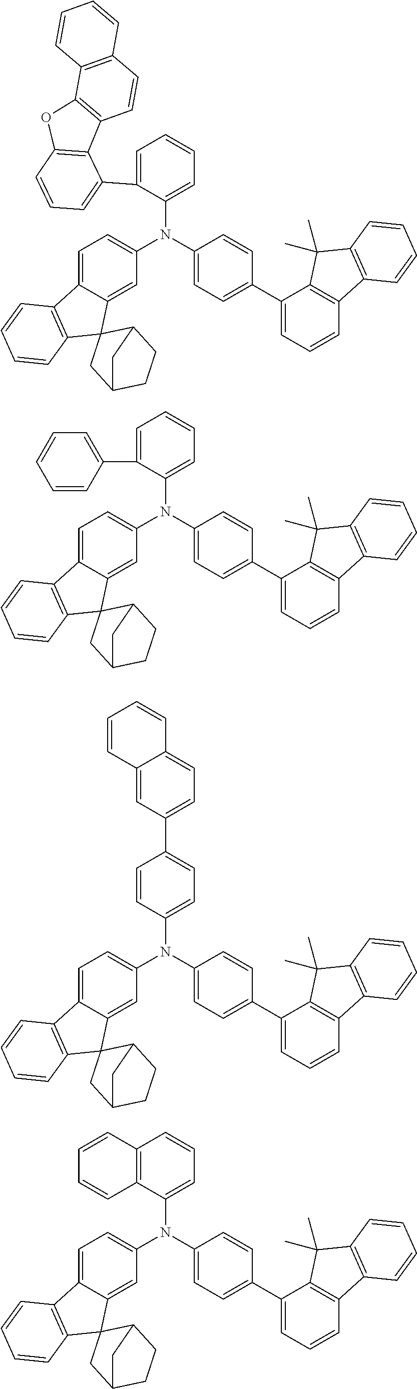

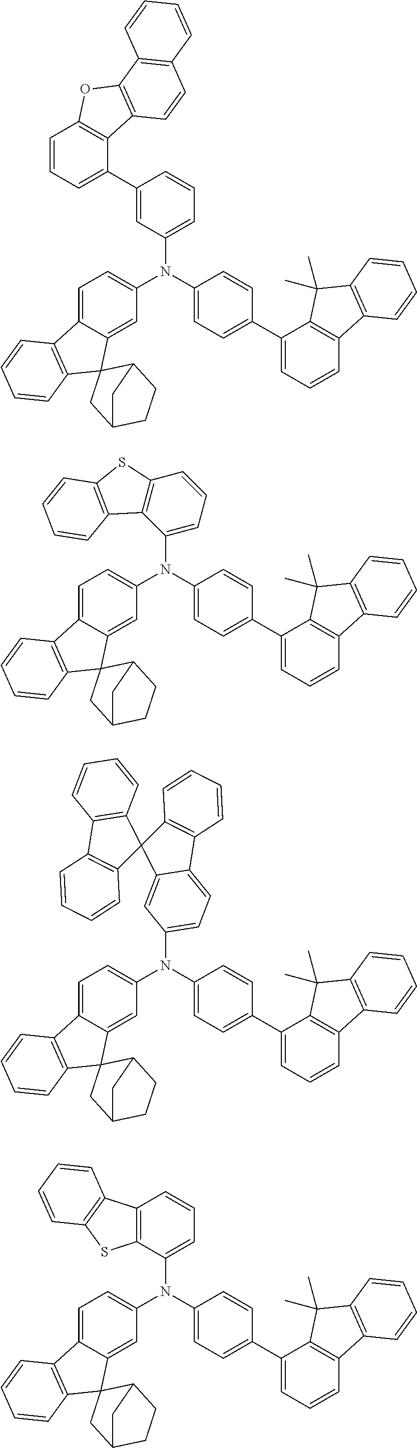

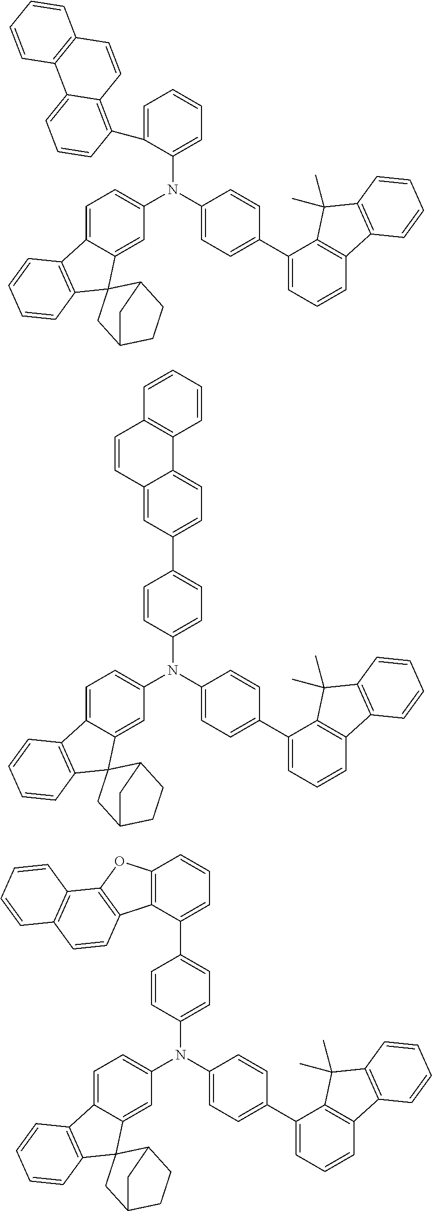

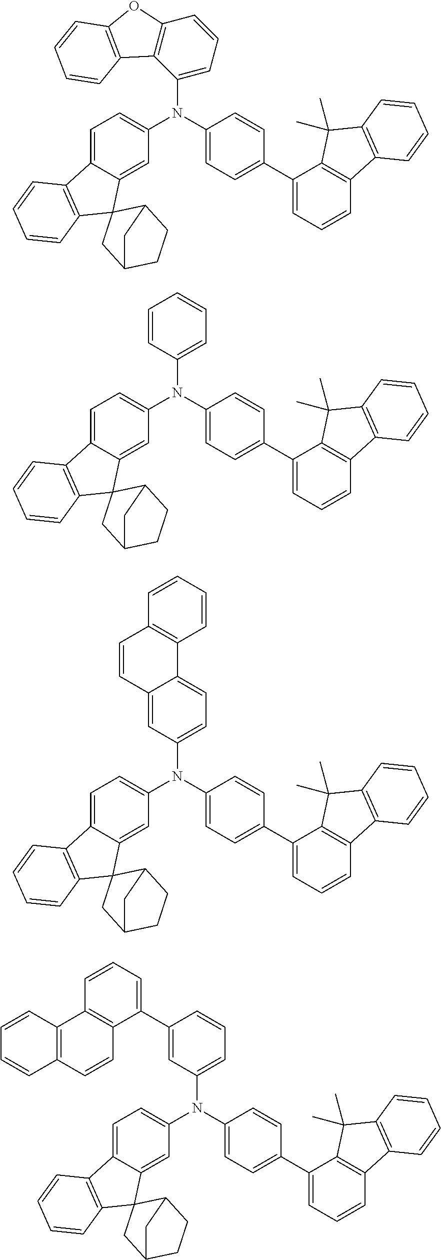

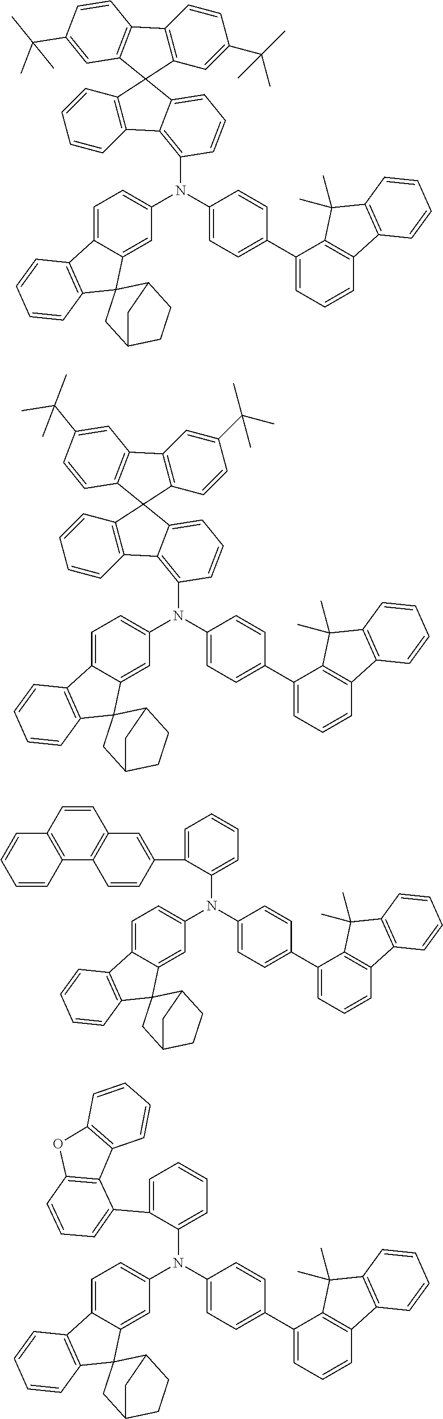 [SMILES: CC(C)(C)c1ccc2c(c1)C1(c3cc(C(C)(C)C)ccc3-2)c2ccccc2-c2c(N(c3ccc(-c4cccc5c4C(C)(C)c4ccccc4-5)cc3)c3ccc4c(c3)C3(CC5CCC3C5)c3ccccc3-4)cccc21 ; CC(C)(C)c1ccc2c(c1)-c1cc(C(C)(C)C)ccc1C21c2ccccc2-c2c(N(c3ccc(-c4cccc5c4C(C)(C)c4ccccc4-5)cc3)c3ccc4c(c3)C3(CC5CCC3C5)c3ccccc3-4)cccc21 ; CC1(C)c2ccccc2-c2cccc(-c3ccc(N(c4ccc5c(c4)C4(CC6CCC4C6)c4ccccc4-5)c4ccccc4-c4ccc5c(ccc6ccccc65)c4)cc3)c21 ; CC1(C)c2ccccc2-c2cccc(-c3ccc(N(c4ccc5c(c4)C4(CC6CCC4C6)c4ccccc4-5)c4ccccc4-c4cccc5oc6ccccc6c45)cc3)c21]

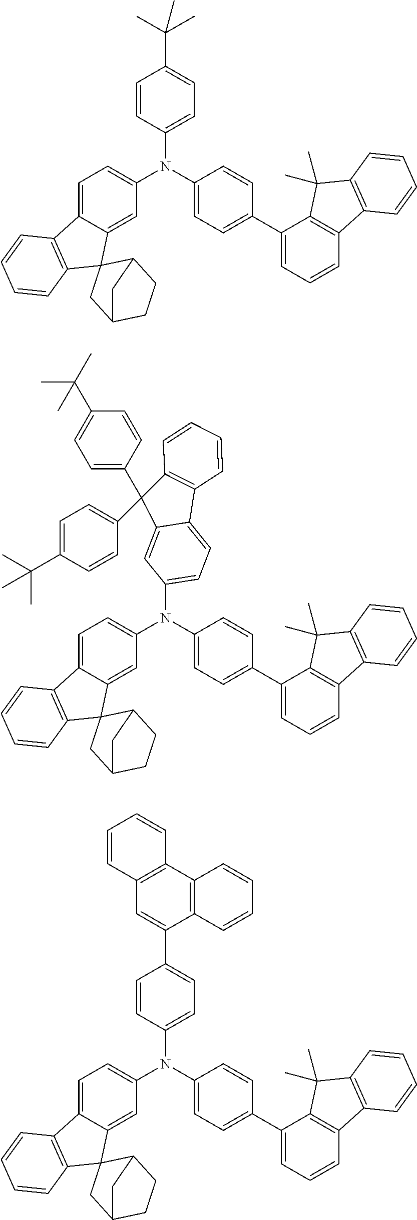

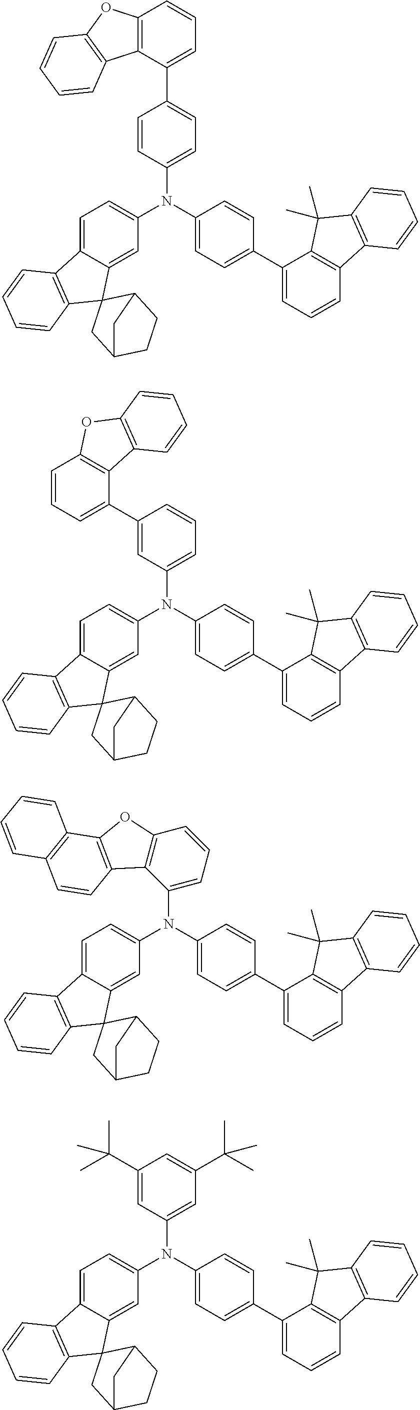

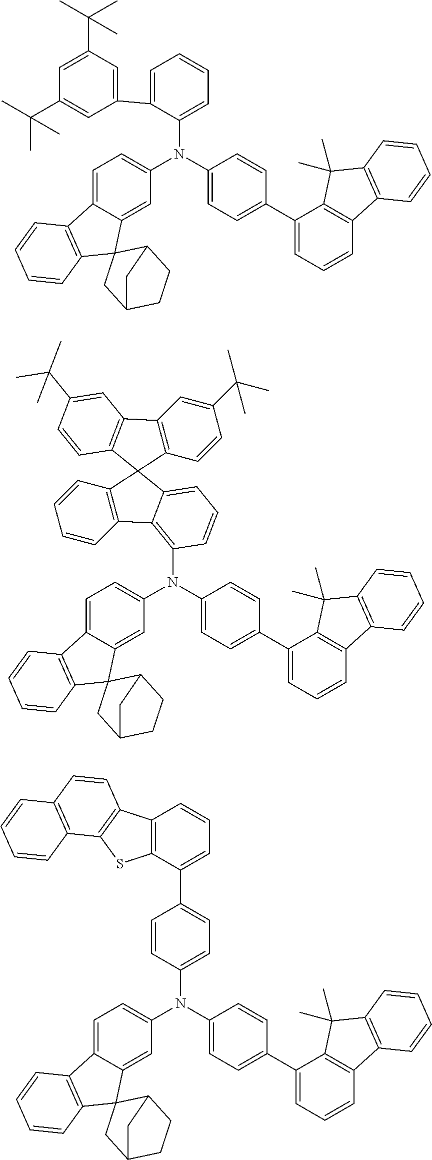

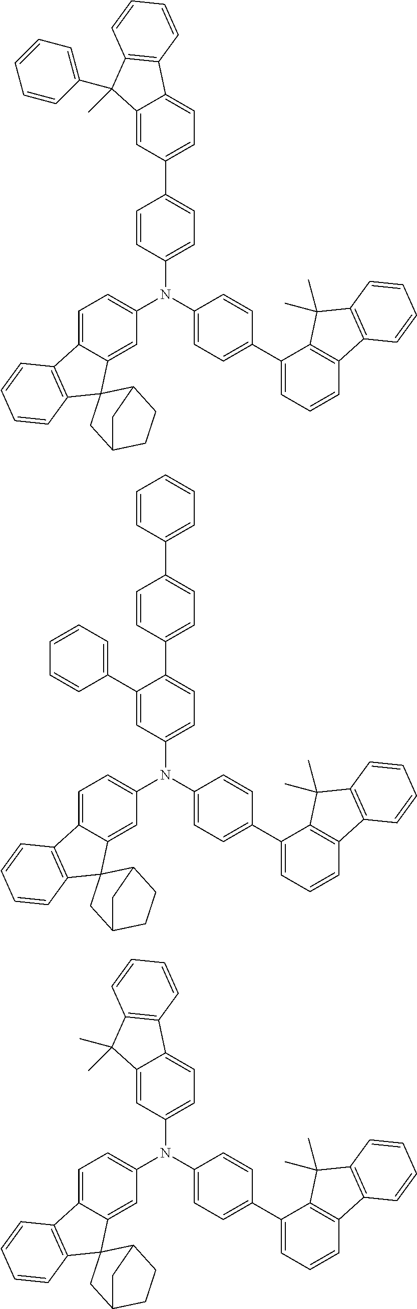

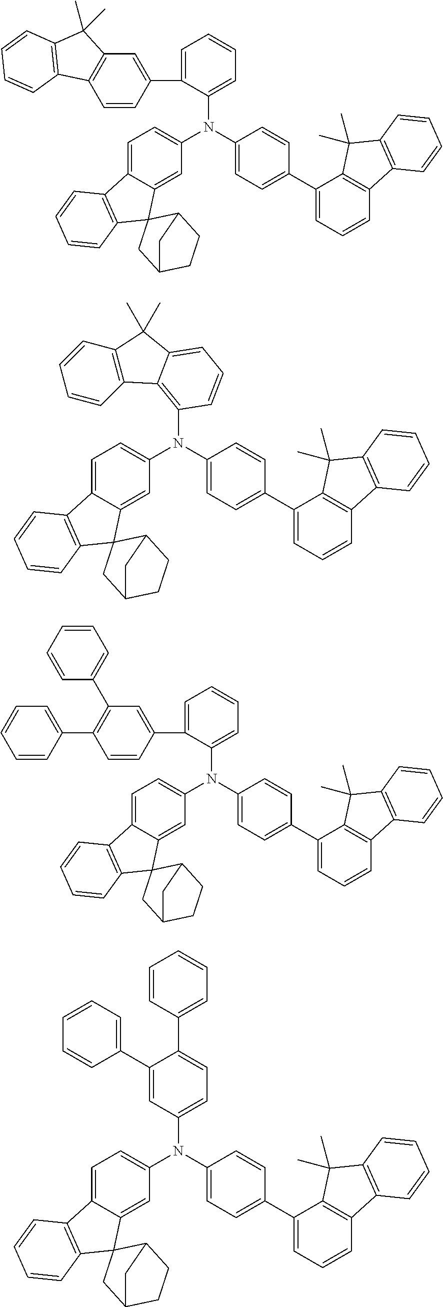

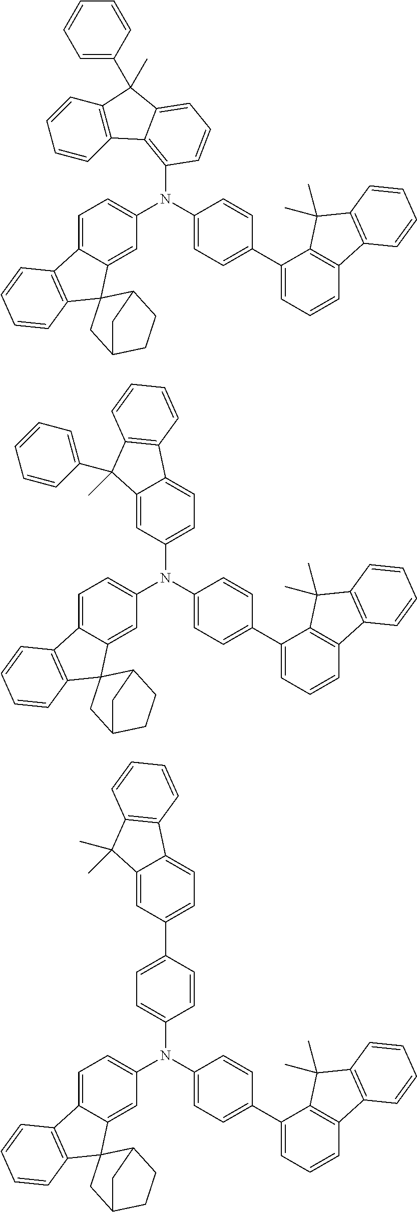

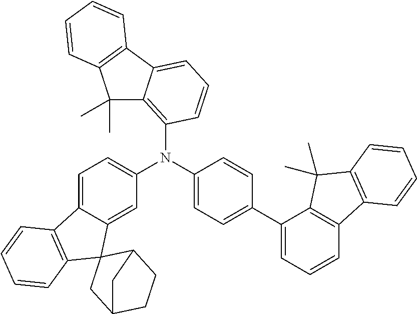 [SMILES: CC1(C)c2ccccc2-c2cccc(-c3ccc(N(c4ccc5c(c4)C4(CC6CCC4C6)c4ccccc4-5)c4cccc5c4C(C)(C)c4ccccc4-5)cc3)c21]

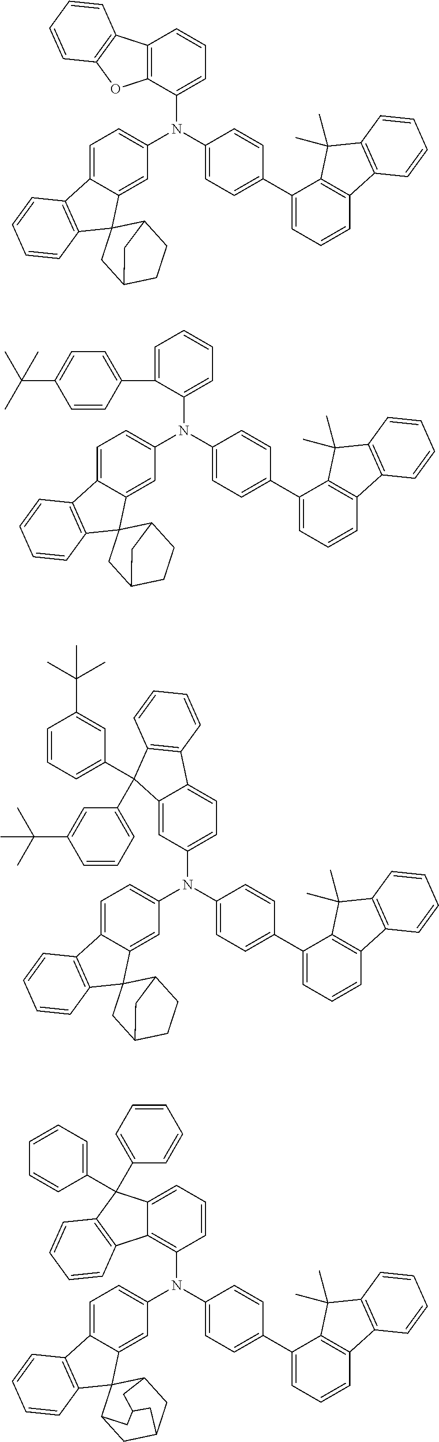 [SMILES: CC1(C)c2ccccc2-c2cccc(-c3ccc(N(c4ccc5c(c4)C4(CC6CCC4C6)c4ccccc4-5)c4cccc5c4oc4ccccc45)cc3)c21 ; CC(C)(C)c1ccc(-c2ccccc2N(c2ccc(-c3cccc4c3C(C)(C)c3ccccc3-4)cc2)c2ccc3c(c2)C2(CC4CCC2C4)c2ccccc2-3)cc1 ; CC(C)(C)c1cccc(C2(c3cccc(C(C)(C)C)c3)c3ccccc3-c3ccc(N(c4ccc(-c5cccc6c5C(C)(C)c5ccccc5-6)cc4)c4ccc5c(c4)C4(CC6CCC4C6)c4ccccc4-5)cc32)c1 ; CC1(C)c2ccccc2-c2cccc(-c3ccc(N(c4ccc5c(c4)C4(c6ccccc6-5)C5CC6CC(C5)CC4C6)c4cccc5c4-c4ccccc4C5(c4ccccc4)c4ccccc4)cc3)c21]

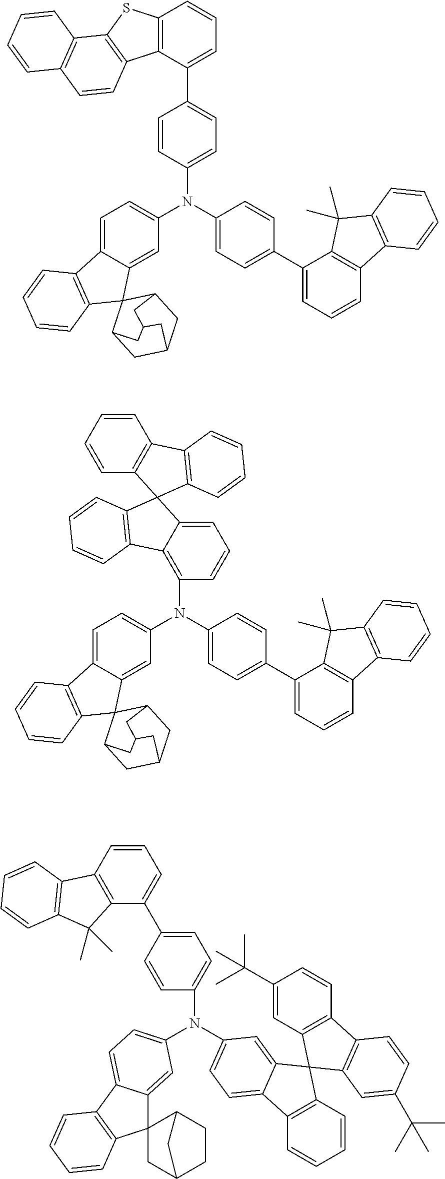 [SMILES: CC1(C)c2ccccc2-c2cccc(-c3ccc(N(c4ccc(-c5cccc6sc7c8ccccc8ccc7c56)cc4)c4ccc5c(c4)C4(c6ccccc6-5)C5CC6CC(C5)CC4C6)cc3)c21 ; CC1(C)c2ccccc2-c2cccc(-c3ccc(N(c4ccc5c(c4)C4(c6ccccc6-5)C5CC6CC(C5)CC4C6)c4cccc5c4-c4ccccc4C54c5ccccc5-c5ccccc54)cc3)c21 ; CC(C)(C)c1ccc2c(c1)C1(c3ccccc3-c3ccc(N(c4ccc(-c5cccc6c5C(C)(C)c5ccccc5-6)cc4)c4ccc5c(c4)C4(CC6CCC4C6)c4ccccc4-5)cc31)c1cc(C(C)(C)C)ccc1-2]

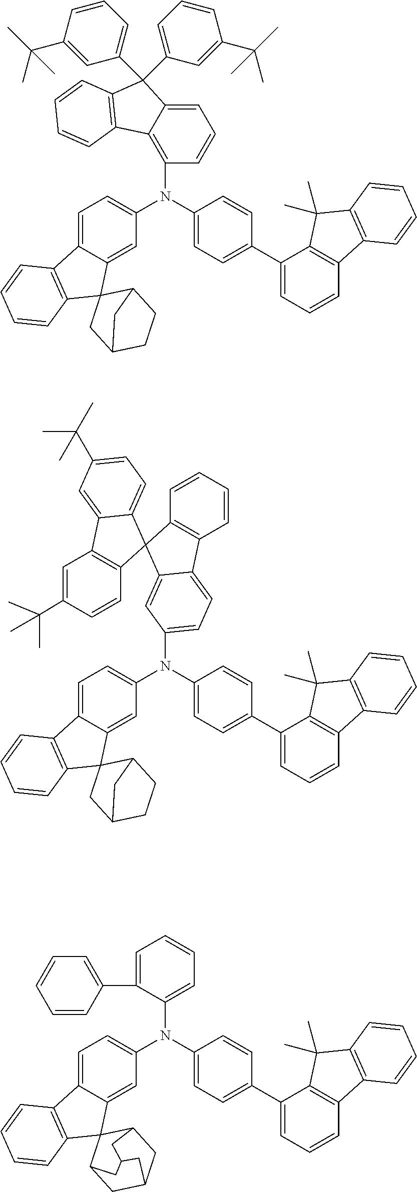

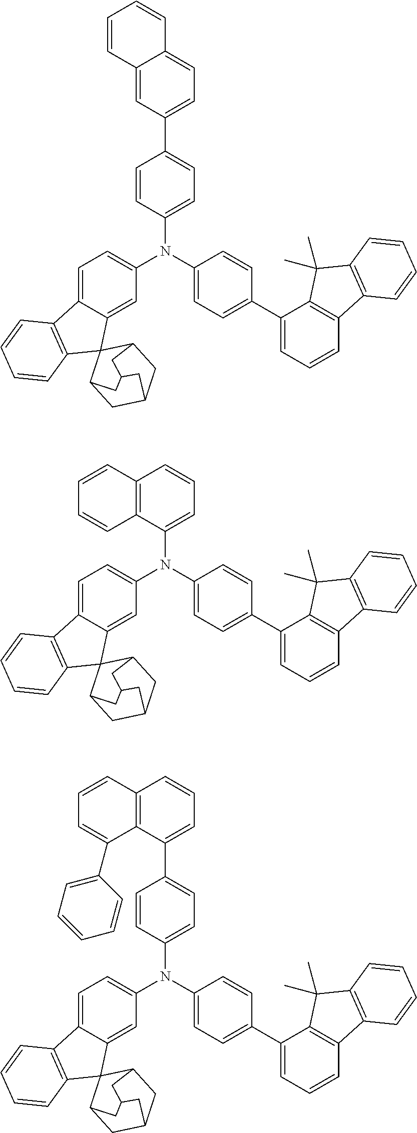 [SMILES: CC1(C)c2ccccc2-c2cccc(-c3ccc(N(c4ccc(-c5ccc6ccccc6c5)cc4)c4ccc5c(c4)C4(c6ccccc6-5)C5CC6CC(C5)CC4C6)cc3)c21 ; CC1(C)c2ccccc2-c2cccc(-c3ccc(N(c4ccc5c(c4)C4(c6ccccc6-5)C5CC6CC(C5)CC4C6)c4cccc5ccccc45)cc3)c21 ; CC1(C)c2ccccc2-c2cccc(-c3ccc(N(c4ccc(-c5cccc6cccc(-c7ccccc7)c56)cc4)c4ccc5c(c4)C4(c6ccccc6-5)C5CC6CC(C5)CC4C6)cc3)c21]

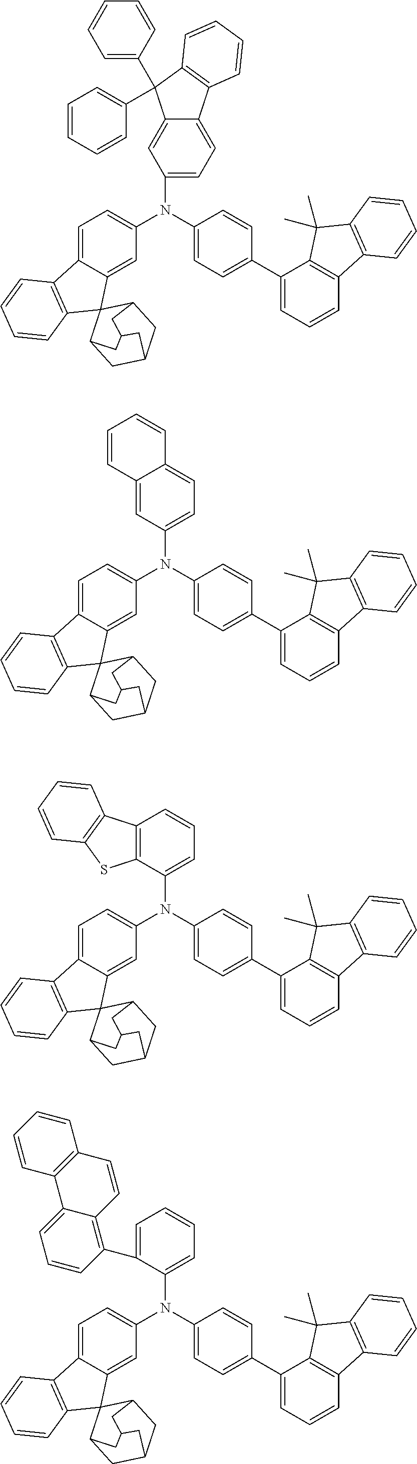 [SMILES: CC1(C)c2ccccc2-c2cccc(-c3ccc(N(c4ccc5c(c4)C(c4ccccc4)(c4ccccc4)c4ccccc4-5)c4ccc5c(c4)C4(c6ccccc6-5)C5CC6CC(C5)CC4C6)cc3)c21 ; CC1(C)c2ccccc2-c2cccc(-c3ccc(N(c4ccc5c(c4)C4(c6ccccc6-5)C5CC6CC(C5)CC4C6)c4ccc5ccccc5c4)cc3)c21 ; CC1(C)c2ccccc2-c2cccc(-c3ccc(N(c4ccc5c(c4)C4(c6ccccc6-5)C5CC6CC(C5)CC4C6)c4cccc5c4sc4ccccc45)cc3)c21 ; CC1(C)c2ccccc2-c2cccc(-c3ccc(N(c4ccc5c(c4)C4(c6ccccc6-5)C5CC6CC(C5)CC4C6)c4ccccc4-c4cccc5c4ccc4ccccc45)cc3)c21]

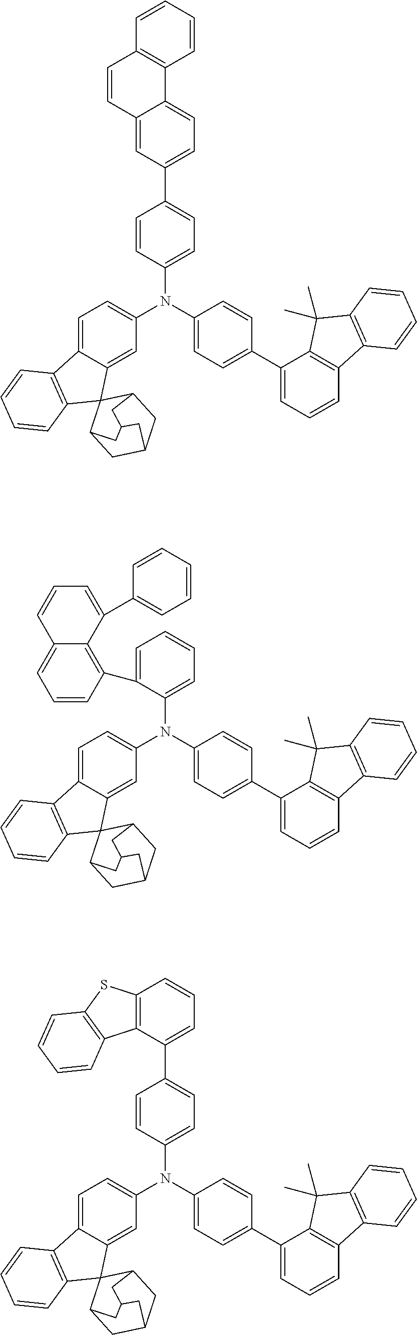 [SMILES: CC1(C)c2ccccc2-c2cccc(-c3ccc(N(c4ccc(-c5ccc6c(ccc7ccccc76)c5)cc4)c4ccc5c(c4)C4(c6ccccc6-5)C5CC6CC(C5)CC4C6)cc3)c21 ; CC1(C)c2ccccc2-c2cccc(-c3ccc(N(c4ccc5c(c4)C4(c6ccccc6-5)C5CC6CC(C5)CC4C6)c4ccccc4-c4cccc5cccc(-c6ccccc6)c45)cc3)c21 ; CC1(C)c2ccccc2-c2cccc(-c3ccc(N(c4ccc(-c5cccc6sc7ccccc7c56)cc4)c4ccc5c(c4)C4(c6ccccc6-5)C5CC6CC(C5)CC4C6)cc3)c21]

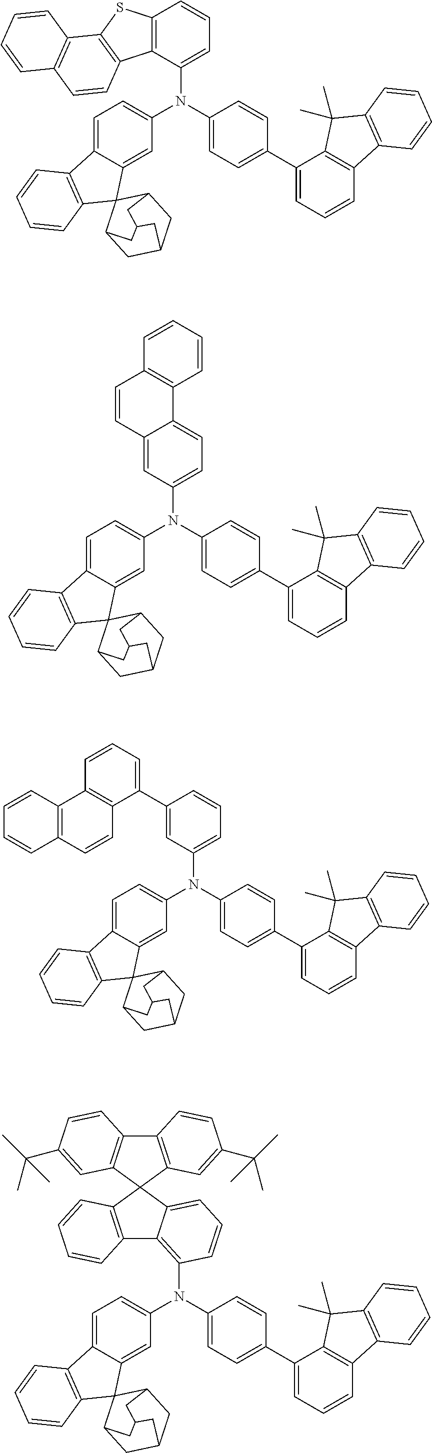 [SMILES: CC1(C)c2ccccc2-c2cccc(-c3ccc(N(c4ccc5c(c4)C4(c6ccccc6-5)C5CC6CC(C5)CC4C6)c4cccc5sc6c7ccccc7ccc6c45)cc3)c21 ; CC1(C)c2ccccc2-c2cccc(-c3ccc(N(c4ccc5c(c4)C4(c6ccccc6-5)C5CC6CC(C5)CC4C6)c4ccc5c(ccc6ccccc65)c4)cc3)c21 ; CC1(C)c2ccccc2-c2cccc(-c3ccc(N(c4cccc(-c5cccc6c5ccc5ccccc56)c4)c4ccc5c(c4)C4(c6ccccc6-5)C5CC6CC(C5)CC4C6)cc3)c21 ; CC(C)(C)c1ccc2c(c1)C1(c3cc(C(C)(C)C)ccc3-2)c2ccccc2-c2c(N(c3ccc(-c4cccc5c4C(C)(C)c4ccccc4-5)cc3)c3ccc4c(c3)C3(c5ccccc5-4)C4CC5CC(C4)CC3C5)cccc21]

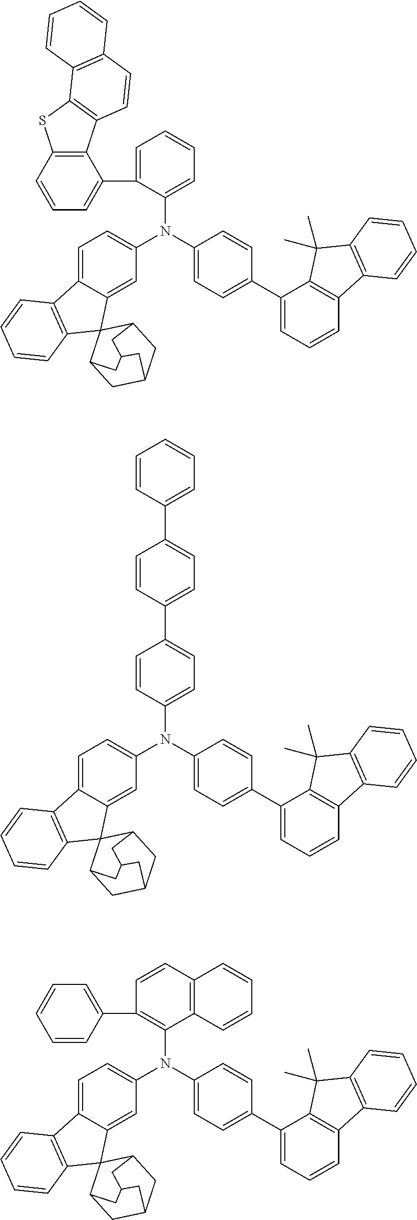 [SMILES: CC1(C)c2ccccc2-c2cccc(-c3ccc(N(c4ccc5c(c4)C4(c6ccccc6-5)C5CC6CC(C5)CC4C6)c4ccccc4-c4cccc5sc6c7ccccc7ccc6c45)cc3)c21 ; CC1(C)c2ccccc2-c2cccc(-c3ccc(N(c4ccc(-c5ccc(-c6ccccc6)cc5)cc4)c4ccc5c(c4)C4(c6ccccc6-5)C5CC6CC(C5)CC4C6)cc3)c21 ; CC1(C)c2ccccc2-c2cccc(-c3ccc(N(c4ccc5c(c4)C4(c6ccccc6-5)C5CC6CC(C5)CC4C6)c4c(-c5ccccc5)ccc5ccccc45)cc3)c21]

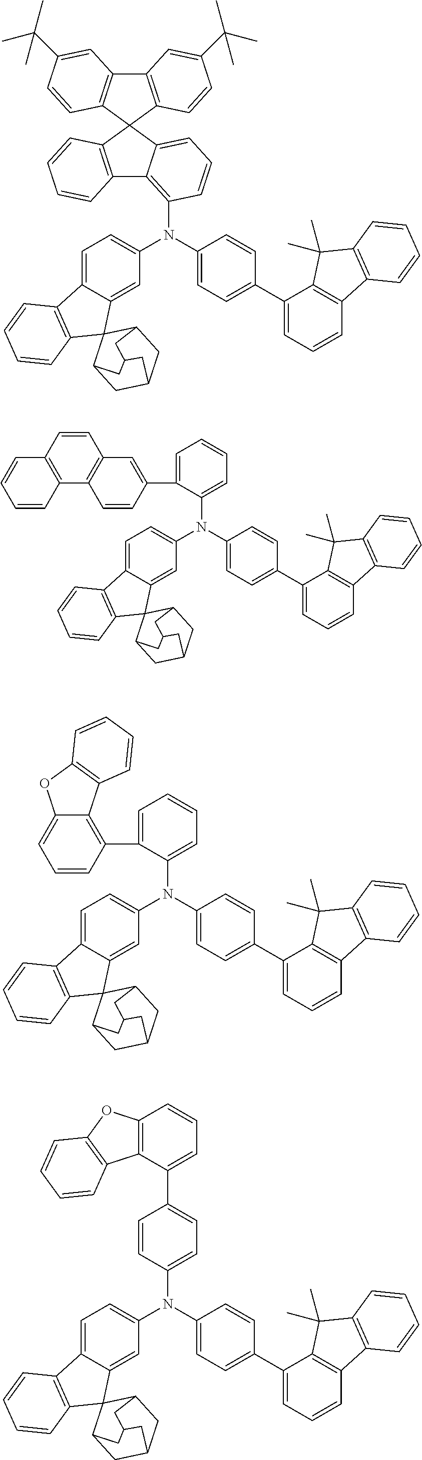 [SMILES: CC(C)(C)c1ccc2c(c1)-c1cc(C(C)(C)C)ccc1C21c2ccccc2-c2c(N(c3ccc(-c4cccc5c4C(C)(C)c4ccccc4-5)cc3)c3ccc4c(c3)C3(c5ccccc5-4)C4CC5CC(C4)CC3C5)cccc21 ; CC1(C)c2ccccc2-c2cccc(-c3ccc(N(c4ccc5c(c4)C4(c6ccccc6-5)C5CC6CC(C5)CC4C6)c4ccccc4-c4ccc5c(ccc6ccccc65)c4)cc3)c21 ; CC1(C)c2ccccc2-c2cccc(-c3ccc(N(c4ccc5c(c4)C4(c6ccccc6-5)C5CC6CC(C5)CC4C6)c4ccccc4-c4cccc5oc6ccccc6c45)cc3)c21 ; CC1(C)c2ccccc2-c2cccc(-c3ccc(N(c4ccc(-c5cccc6oc7ccccc7c56)cc4)c4ccc5c(c4)C4(c6ccccc6-5)C5CC6CC(C5)CC4C6)cc3)c21]

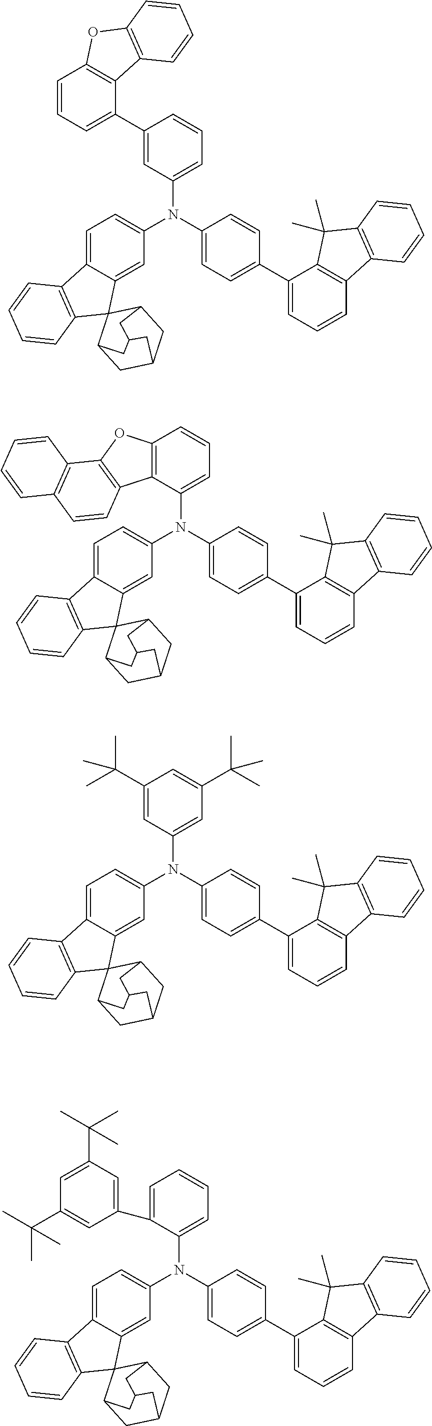 [SMILES: CC1(C)c2ccccc2-c2cccc(-c3ccc(N(c4cccc(-c5cccc6oc7ccccc7c56)c4)c4ccc5c(c4)C4(c6ccccc6-5)C5CC6CC(C5)CC4C6)cc3)c21 ; CC1(C)c2ccccc2-c2cccc(-c3ccc(N(c4ccc5c(c4)C4(c6ccccc6-5)C5CC6CC(C5)CC4C6)c4cccc5oc6c7ccccc7ccc6c45)cc3)c21 ; CC(C)(C)c1cc(N(c2ccc(-c3cccc4c3C(C)(C)c3ccccc3-4)cc2)c2ccc3c(c2)C2(c4ccccc4-3)C3CC4CC(C3)CC2C4)cc(C(C)(C)C)c1 ; CC(C)(C)c1cc(-c2ccccc2N(c2ccc(-c3cccc4c3C(C)(C)c3ccccc3-4)cc2)c2ccc3c(c2)C2(c4ccccc4-3)C3CC4CC(C3)CC2C4)cc(C(C)(C)C)c1]

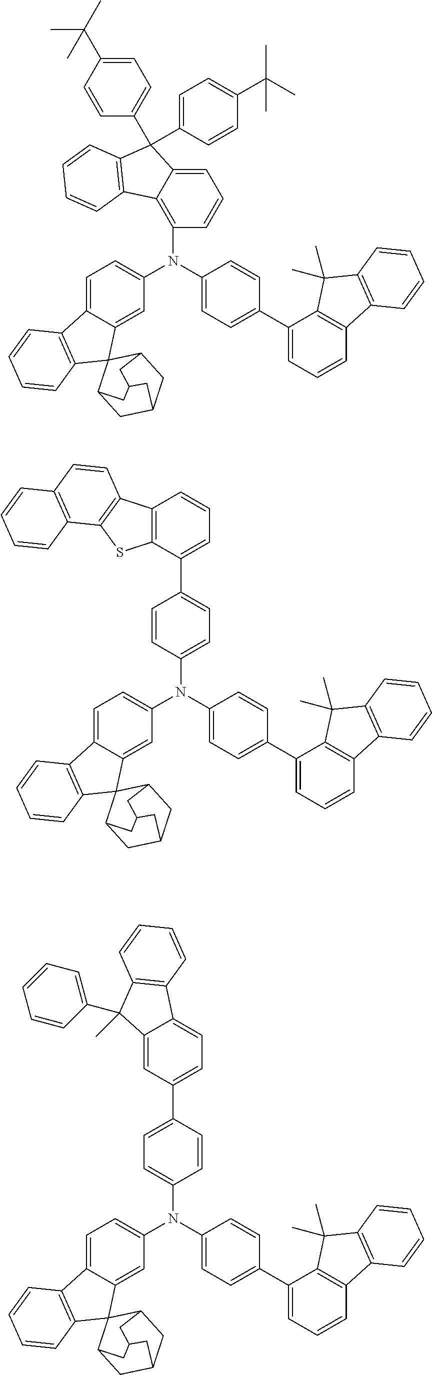 [SMILES: CC(C)(C)c1ccc(C2(c3ccc(C(C)(C)C)cc3)c3ccccc3-c3c(N(c4ccc(-c5cccc6c5C(C)(C)c5ccccc5-6)cc4)c4ccc5c(c4)C4(c6ccccc6-5)C5CC6CC(C5)CC4C6)cccc32)cc1 ; CC1(C)c2ccccc2-c2cccc(-c3ccc(N(c4ccc(-c5cccc6c5sc5c7ccccc7ccc65)cc4)c4ccc5c(c4)C4(c6ccccc6-5)C5CC6CC(C5)CC4C6)cc3)c21 ; CC1(C)c2ccccc2-c2cccc(-c3ccc(N(c4ccc(-c5ccc6c(c5)C(C)(c5ccccc5)c5ccccc5-6)cc4)c4ccc5c(c4)C4(c6ccccc6-5)C5CC6CC(C5)CC4C6)cc3)c21]

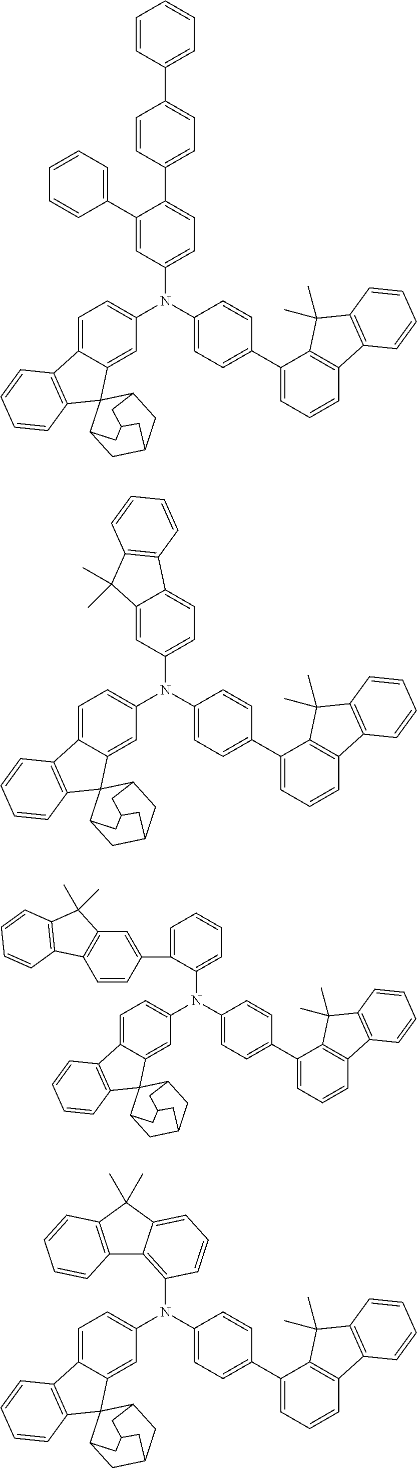

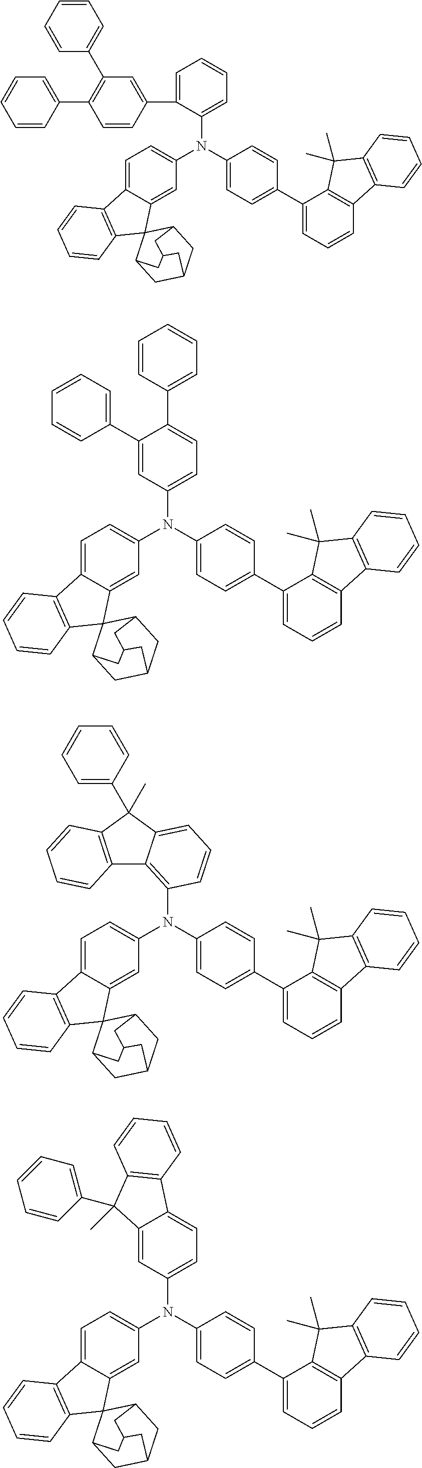

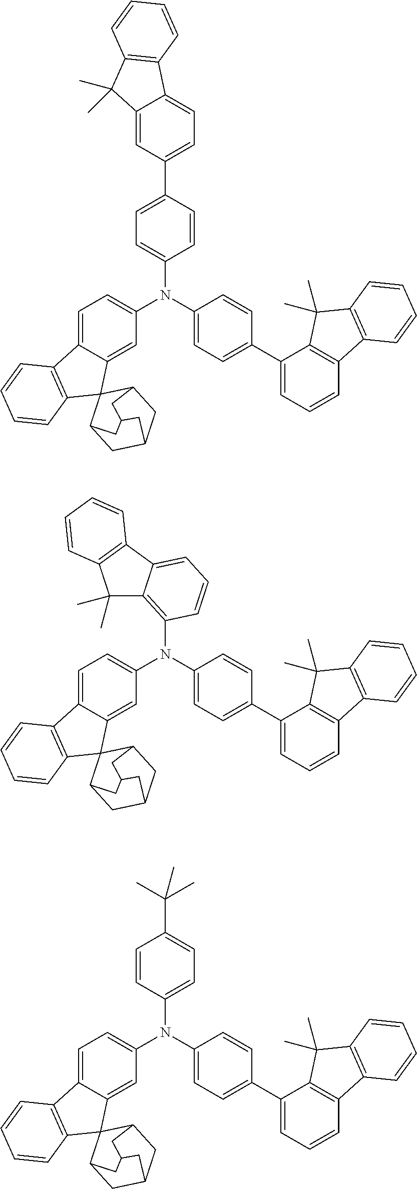 [SMILES: CC1(C)c2ccccc2-c2ccc(-c3ccc(N(c4ccc(-c5cccc6c5C(C)(C)c5ccccc5-6)cc4)c4ccc5c(c4)C4(c6ccccc6-5)C5CC6CC(C5)CC4C6)cc3)cc21 ; CC1(C)c2ccccc2-c2cccc(-c3ccc(N(c4ccc5c(c4)C4(c6ccccc6-5)C5CC6CC(C5)CC4C6)c4cccc5c4C(C)(C)c4ccccc4-5)cc3)c21 ; CC(C)(C)c1ccc(N(c2ccc(-c3cccc4c3C(C)(C)c3ccccc3-4)cc2)c2ccc3c(c2)C2(c4ccccc4-3)C3CC4CC(C3)CC2C4)cc1]

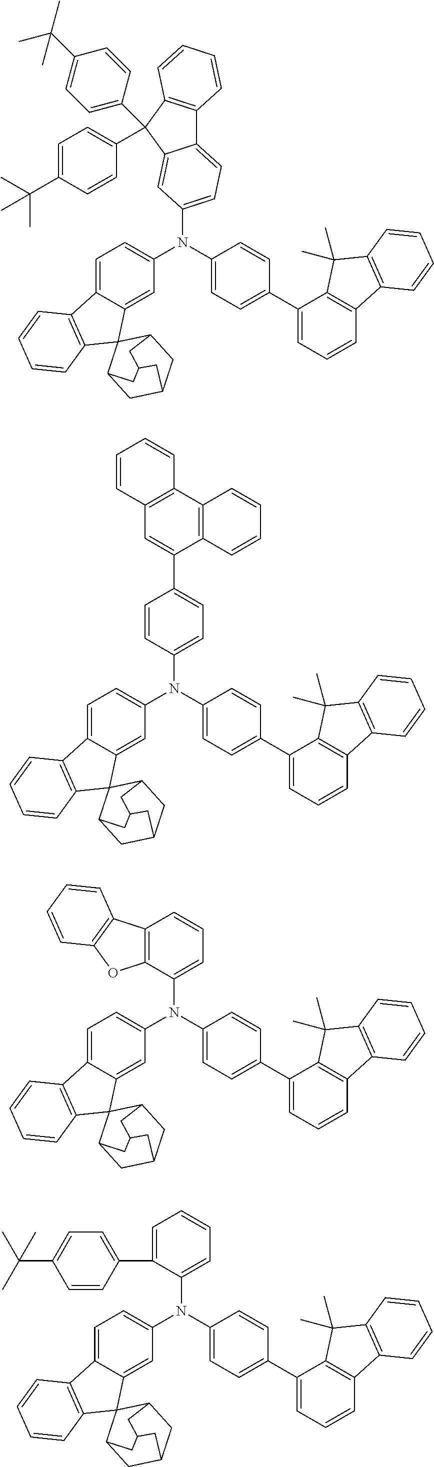

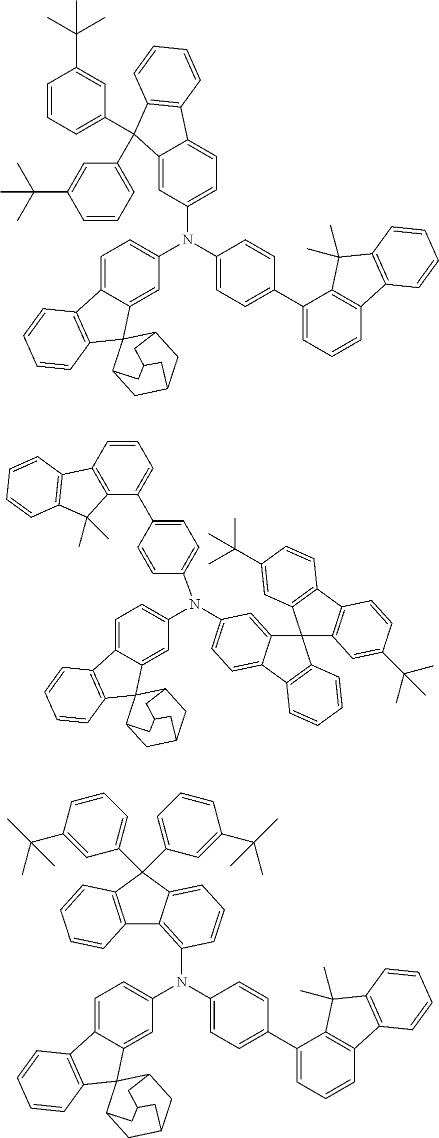

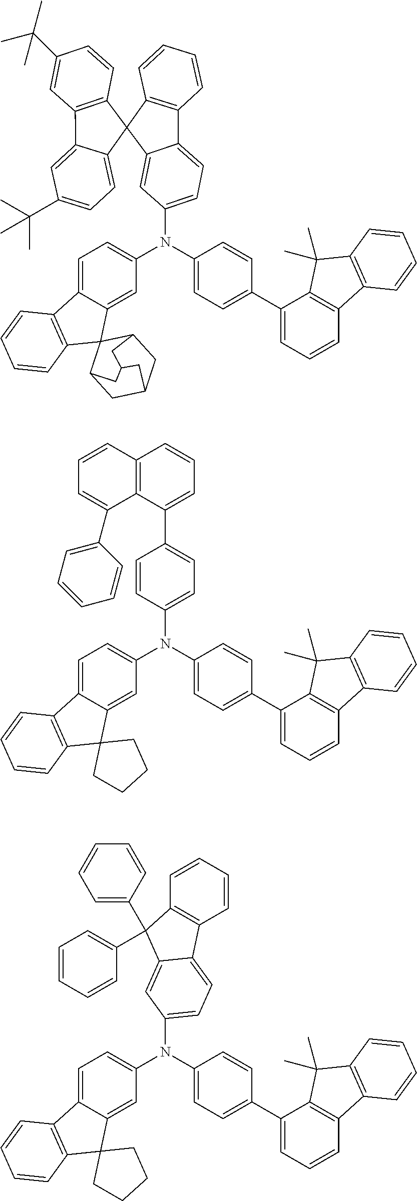

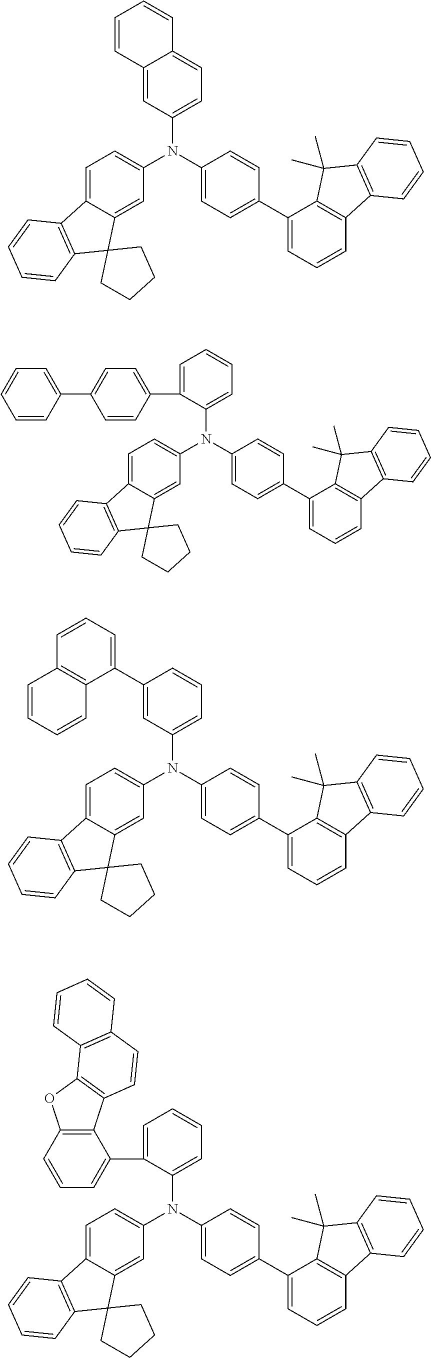 [SMILES: CC1(C)c2ccccc2-c2cccc(-c3ccc(N(c4ccc5c(c4)C4(CCCC4)c4ccccc4-5)c4ccc5ccccc5c4)cc3)c21 ; CC1(C)c2ccccc2-c2cccc(-c3ccc(N(c4ccc5c(c4)C4(CCCC4)c4ccccc4-5)c4ccccc4-c4ccc(-c5ccccc5)cc4)cc3)c21 ; CC1(C)c2ccccc2-c2cccc(-c3ccc(N(c4cccc(-c5cccc6ccccc56)c4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4ccccc4-c4cccc5oc6c7ccccc7ccc6c45)cc3)c21]

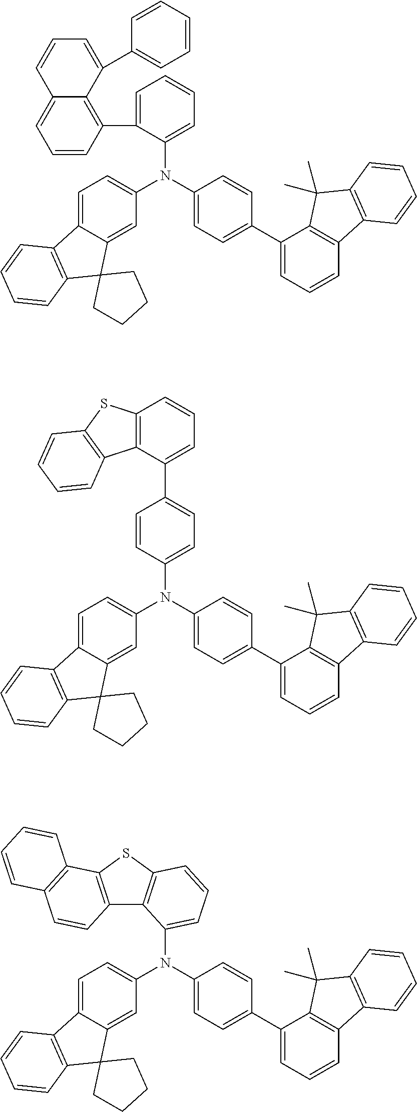 [SMILES: CC1(C)c2ccccc2-c2cccc(-c3ccc(N(c4ccc5c(c4)C4(CCCC4)c4ccccc4-5)c4ccccc4-c4cccc5cccc(-c6ccccc6)c45)cc3)c21 ; CC1(C)c2ccccc2-c2cccc(-c3ccc(N(c4ccc(-c5cccc6sc7ccccc7c56)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4cccc5sc6c7ccccc7ccc6c45)cc3)c21]

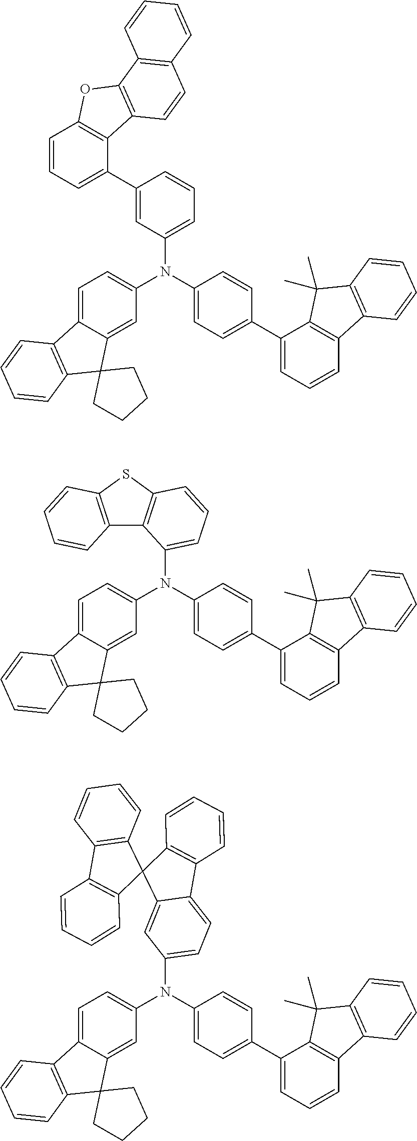 [SMILES: CC1(C)c2ccccc2-c2cccc(-c3ccc(N(c4cccc(-c5cccc6oc7c8ccccc8ccc7c56)c4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4cccc5sc6ccccc6c45)cc3)c21 ; CC1(C)c2ccccc2-c2cccc(-c3ccc(N(c4ccc5c(c4)C4(CCCC4)c4ccccc4-5)c4ccc5c(c4)C4(c6ccccc6-c6ccccc64)c4ccccc4-5)cc3)c21]

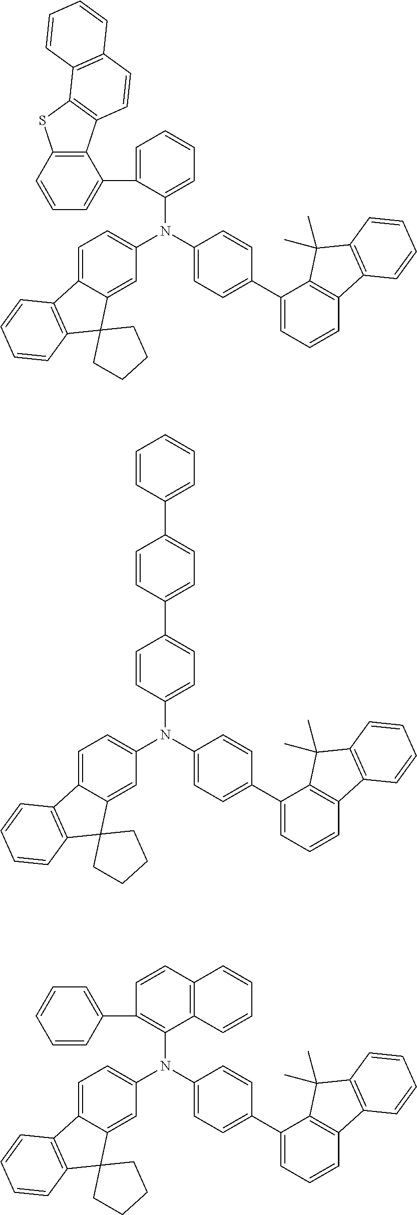 [SMILES: CC1(C)c2ccccc2-c2cccc(-c3ccc(N(c4ccc5c(c4)C4(CCCC4)c4ccccc4-5)c4ccccc4-c4cccc5sc6c7ccccc7ccc6c45)cc3)c21 ; CC1(C)c2ccccc2-c2cccc(-c3ccc(N(c4ccc(-c5ccc(-c6ccccc6)cc5)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4c(-c5ccccc5)ccc5ccccc45)cc3)c21]

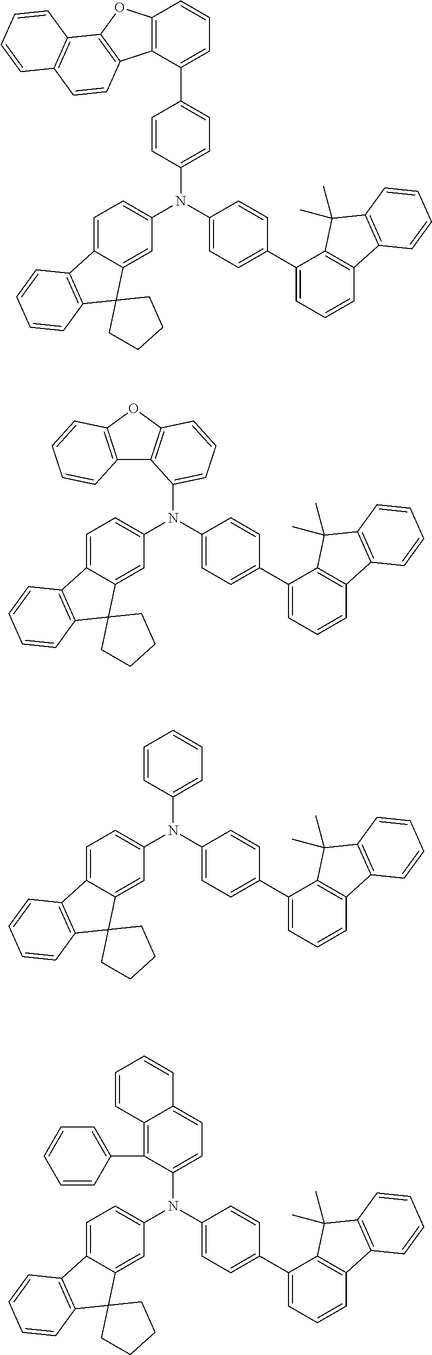 [SMILES: CC1(C)c2ccccc2-c2cccc(-c3ccc(N(c4ccc(-c5cccc6oc7c8ccccc8ccc7c56)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4cccc5oc6ccccc6c45)cc3)c21 ; CC1(C)c2ccccc2-c2cccc(-c3ccc(N(c4ccccc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4ccc5ccccc5c4-c4ccccc4)cc3)c21]

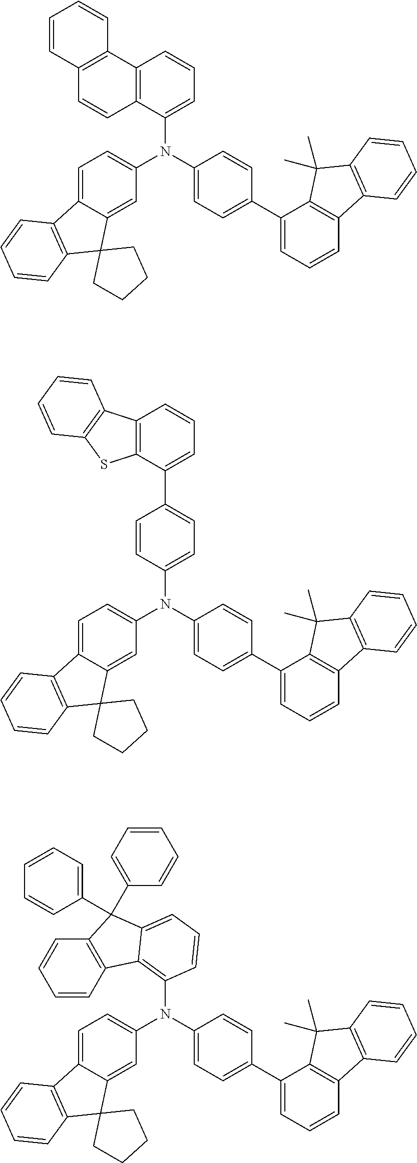 [SMILES: CC1(C)c2ccccc2-c2cccc(-c3ccc(N(c4ccc5c(c4)C4(CCCC4)c4ccccc4-5)c4cccc5c4ccc4ccccc45)cc3)c21 ; CC1(C)c2ccccc2-c2cccc(-c3ccc(N(c4ccc(-c5cccc6c5sc5ccccc56)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4cccc5c4-c4ccccc4C5(c4ccccc4)c4ccccc4)cc3)c21]

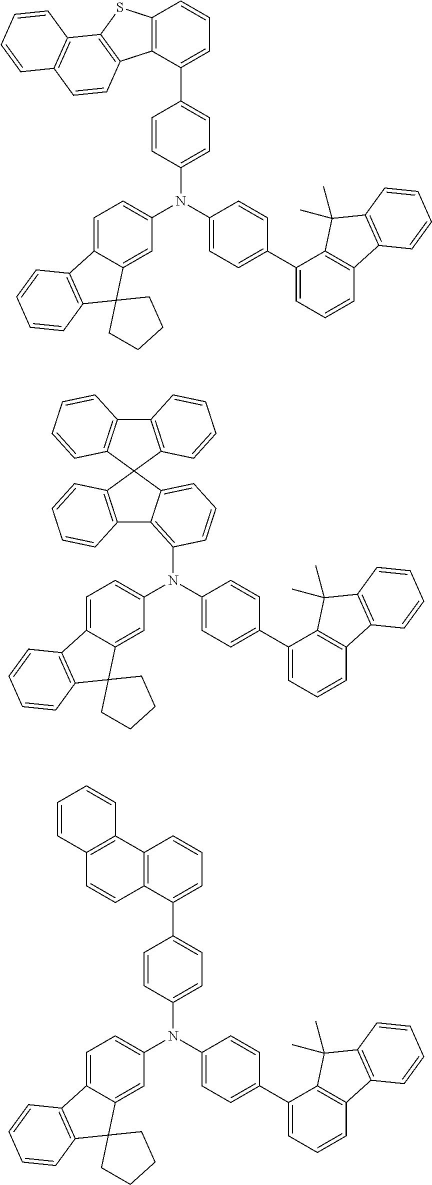 [SMILES: CC1(C)c2ccccc2-c2cccc(-c3ccc(N(c4ccc(-c5cccc6sc7c8ccccc8ccc7c56)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4cccc5c4-c4ccccc4C54c5ccccc5-c5ccccc54)cc3)c21 ; CC1(C)c2ccccc2-c2cccc(-c3ccc(N(c4ccc(-c5cccc6c5ccc5ccccc56)cc4)c4ccc5c(c4)C4(CCCC4)c4ccccc4-5)cc3)c21]

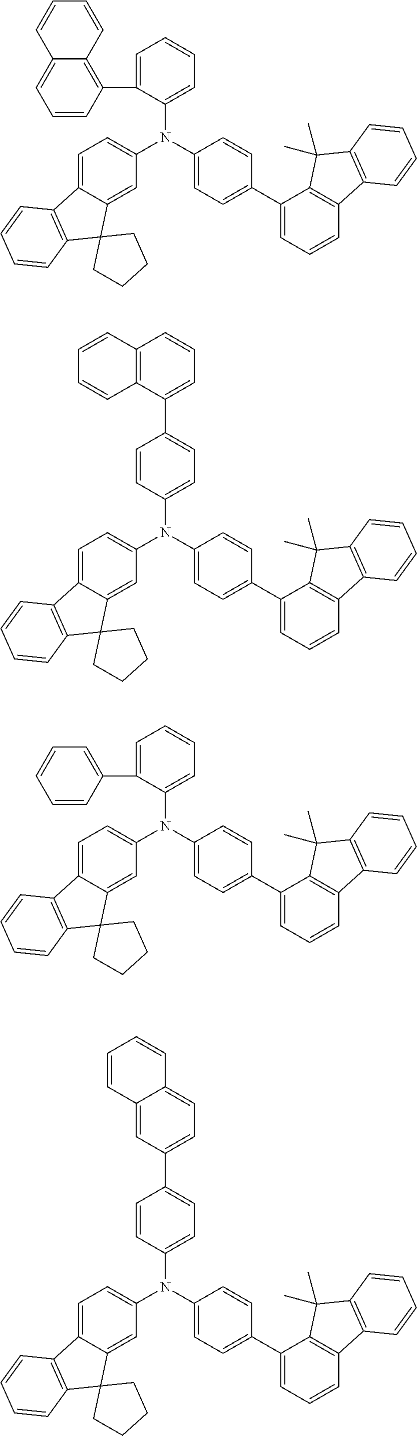 [SMILES: CC1(C)c2ccccc2-c2cccc(-c3ccc(N(c4ccc5c(c4)C4(CCCC4)c4ccccc4-5)c4ccccc4-c4cccc5ccccc45)cc3)c21 ; CC1(C)c2ccccc2-c2cccc(-c3ccc(N(c4ccc(-c5cccc6ccccc56)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4ccccc4-c4ccccc4)cc3)c21 ; CC1(C)c2ccccc2-c2cccc(-c3ccc(N(c4ccc(-c5ccc6ccccc6c5)cc4)c4ccc5c(c4)C4(CCCC4)c4ccccc4-5)cc3)c21]

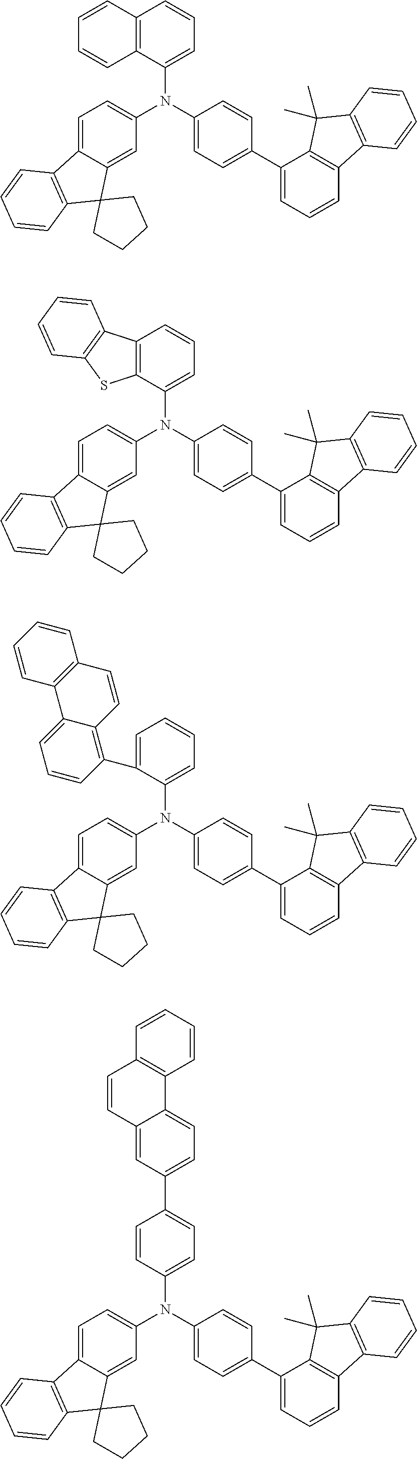 [SMILES: CC1(C)c2ccccc2-c2cccc(-c3ccc(N(c4ccc5c(c4)C4(CCCC4)c4ccccc4-5)c4cccc5ccccc45)cc3)c21 ; CC1(C)c2ccccc2-c2cccc(-c3ccc(N(c4ccc5c(c4)C4(CCCC4)c4ccccc4-5)c4cccc5c4sc4ccccc45)cc3)c21 ; CC1(C)c2ccccc2-c2cccc(-c3ccc(N(c4ccc5c(c4)C4(CCCC4)c4ccccc4-5)c4ccccc4-c4cccc5c4ccc4ccccc45)cc3)c21 ; CC1(C)c2ccccc2-c2cccc(-c3ccc(N(c4ccc(-c5ccc6c(ccc7ccccc76)c5)cc4)c4ccc5c(c4)C4(CCCC4)c4ccccc4-5)cc3)c21]

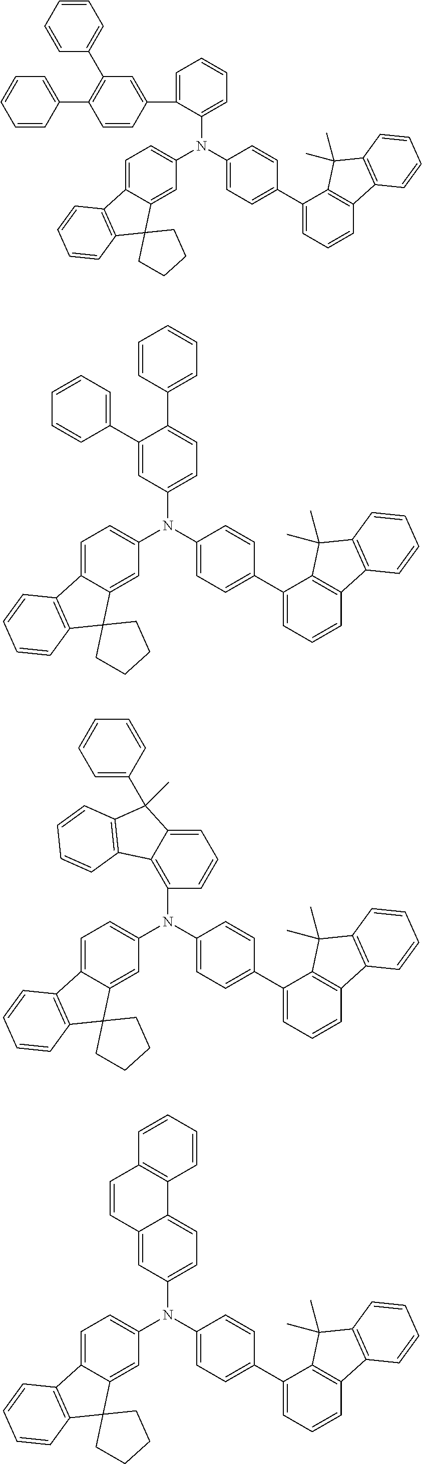

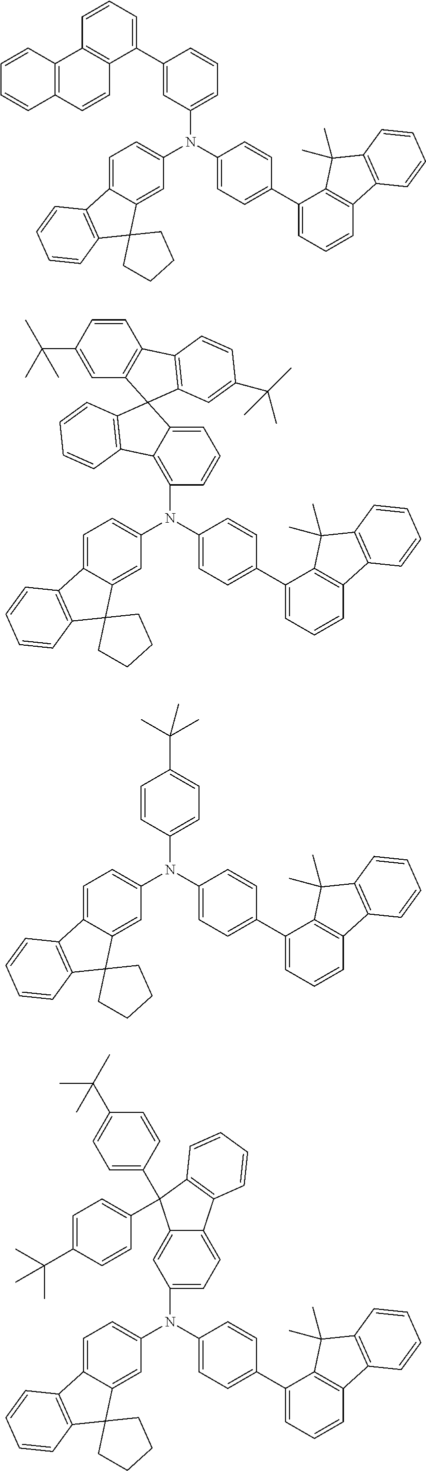

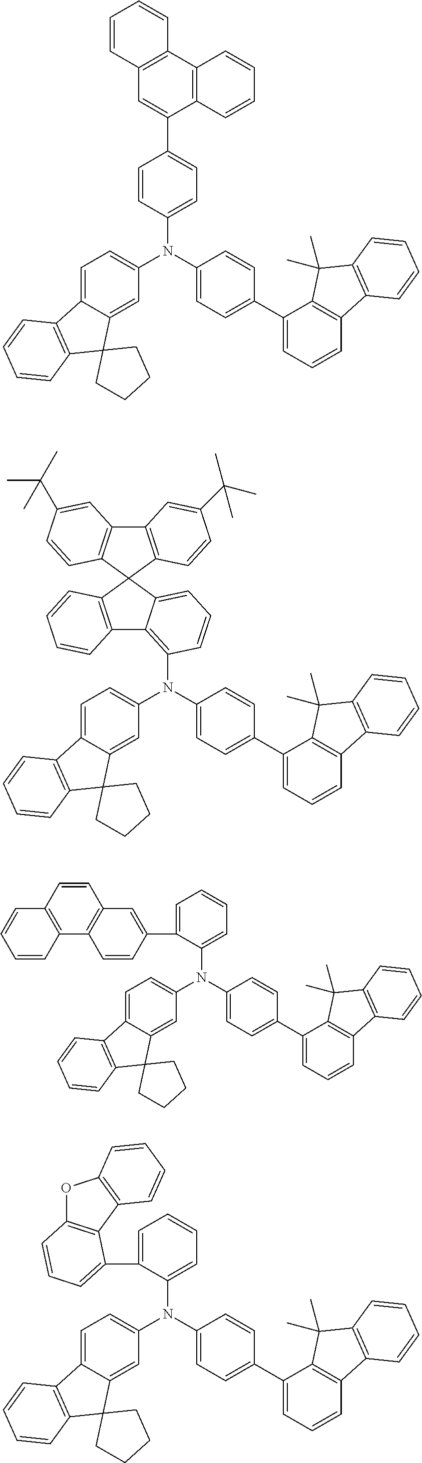

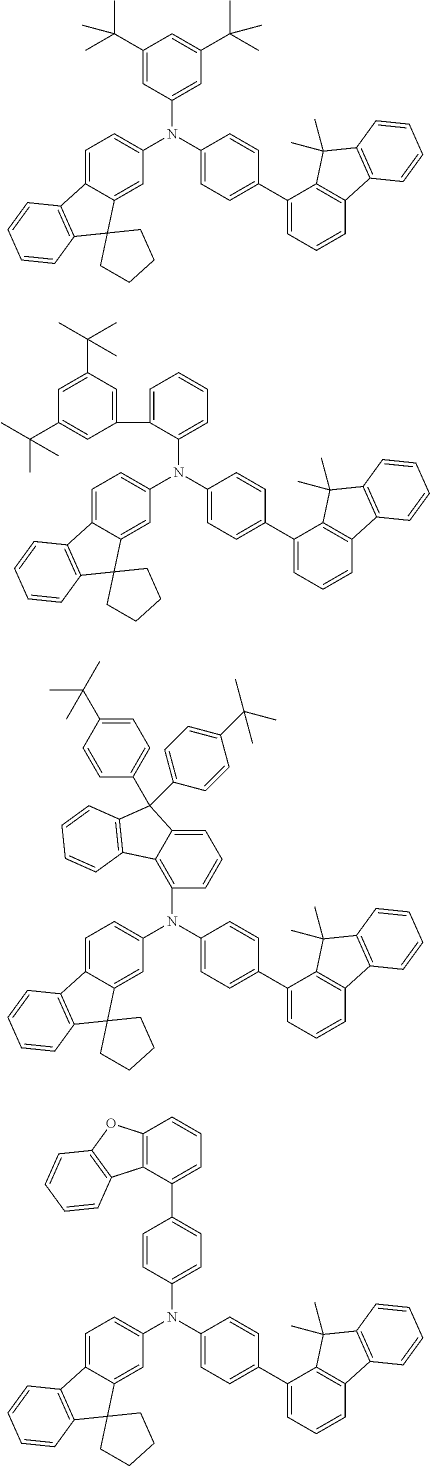

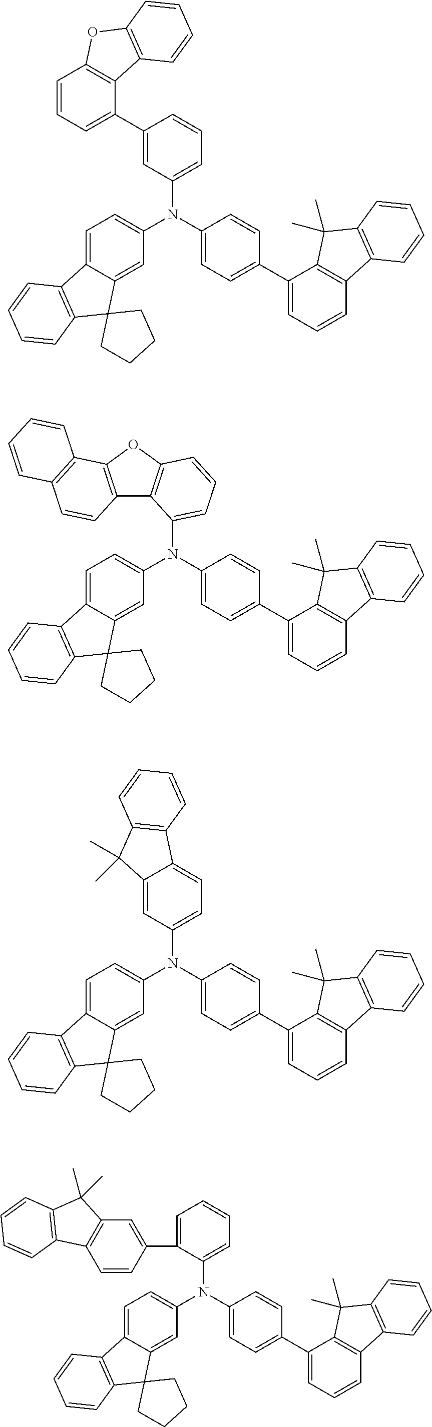 [SMILES: CC1(C)c2ccccc2-c2cccc(-c3ccc(N(c4cccc(-c5cccc6oc7ccccc7c56)c4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4cccc5oc6c7ccccc7ccc6c45)cc3)c21 ; CC1(C)c2ccccc2-c2ccc(N(c3ccc(-c4cccc5c4C(C)(C)c4ccccc4-5)cc3)c3ccc4c(c3)C3(CCCC3)c3ccccc3-4)cc21 ; CC1(C)c2ccccc2-c2ccc(-c3ccccc3N(c3ccc(-c4cccc5c4C(C)(C)c4ccccc4-5)cc3)c3ccc4c(c3)C3(CCCC3)c3ccccc3-4)cc21]

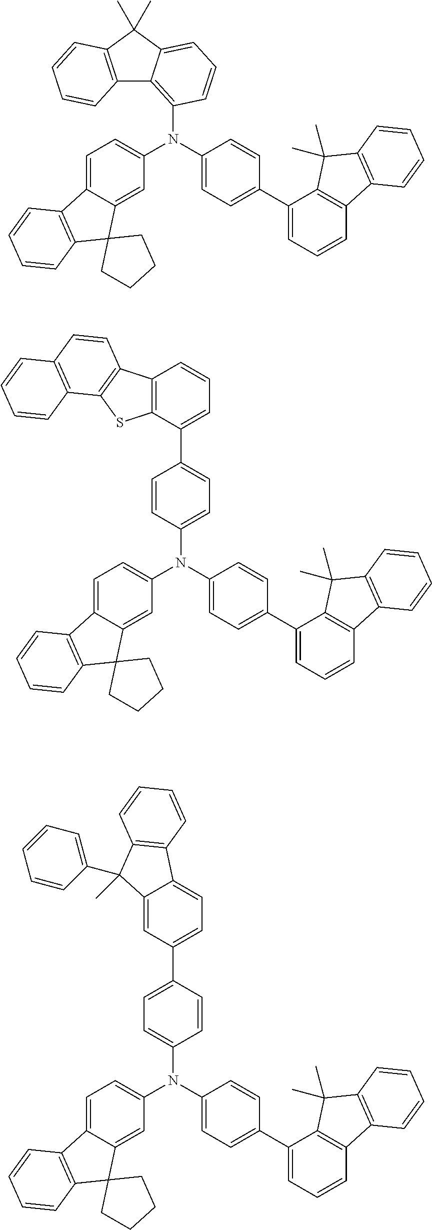 [SMILES: CC1(C)c2ccccc2-c2c(N(c3ccc(-c4cccc5c4C(C)(C)c4ccccc4-5)cc3)c3ccc4c(c3)C3(CCCC3)c3ccccc3-4)cccc21 ; CC1(C)c2ccccc2-c2cccc(-c3ccc(N(c4ccc(-c5cccc6c5sc5c7ccccc7ccc65)cc4)c4ccc5c(c4)C4(CCCC4)c4ccccc4-5)cc3)c21 ; CC1(C)c2ccccc2-c2cccc(-c3ccc(N(c4ccc(-c5ccc6c(c5)C(C)(c5ccccc5)c5ccccc5-6)cc4)c4ccc5c(c4)C4(CCCC4)c4ccccc4-5)cc3)c21]

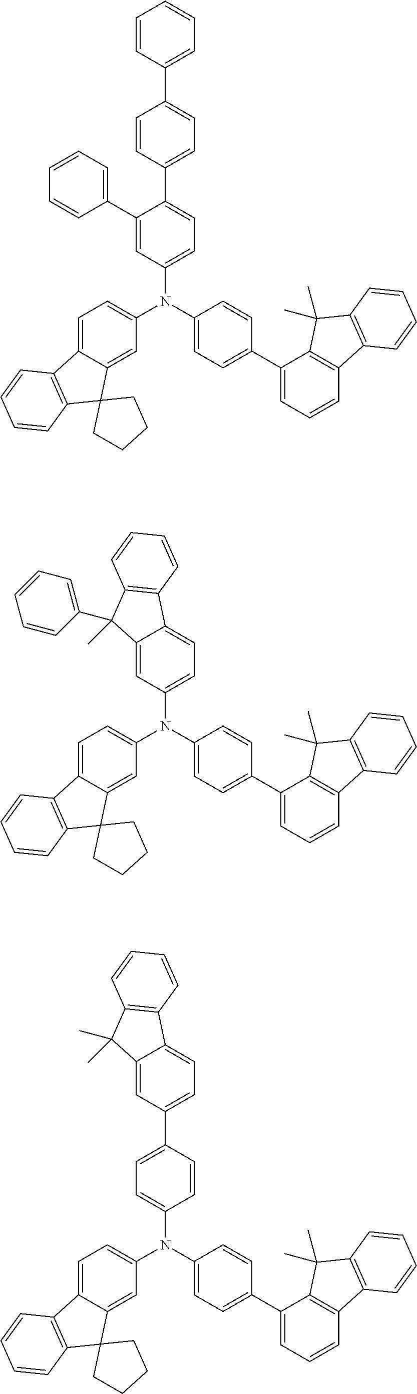 [SMILES: CC1(C)c2ccccc2-c2cccc(-c3ccc(N(c4ccc(-c5ccc(-c6ccccc6)cc5)c(-c5ccccc5)c4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4ccc5c(c4)C(C)(c4ccccc4)c4ccccc4-5)cc3)c21 ; CC1(C)c2ccccc2-c2ccc(-c3ccc(N(c4ccc(-c5cccc6c5C(C)(C)c5ccccc5-6)cc4)c4ccc5c(c4)C4(CCCC4)c4ccccc4-5)cc3)cc21]

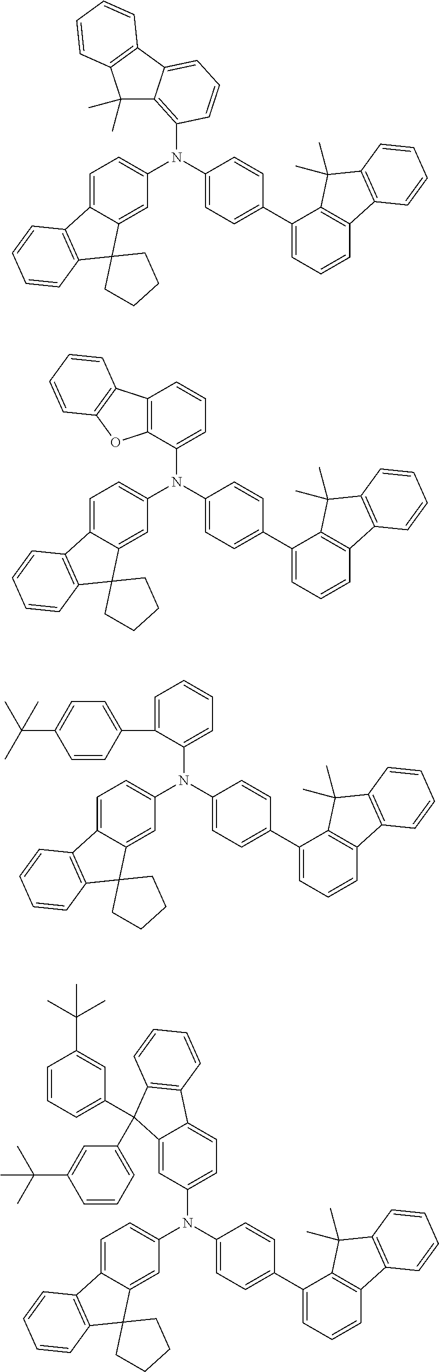 [SMILES: CC1(C)c2ccccc2-c2cccc(-c3ccc(N(c4ccc5c(c4)C4(CCCC4)c4ccccc4-5)c4cccc5c4C(C)(C)c4ccccc4-5)cc3)c21 ; CC1(C)c2ccccc2-c2cccc(-c3ccc(N(c4ccc5c(c4)C4(CCCC4)c4ccccc4-5)c4cccc5c4oc4ccccc45)cc3)c21 ; CC(C)(C)c1ccc(-c2ccccc2N(c2ccc(-c3cccc4c3C(C)(C)c3ccccc3-4)cc2)c2ccc3c(c2)C2(CCCC2)c2ccccc2-3)cc1 ; CC(C)(C)c1cccc(C2(c3cccc(C(C)(C)C)c3)c3ccccc3-c3ccc(N(c4ccc(-c5cccc6c5C(C)(C)c5ccccc5-6)cc4)c4ccc5c(c4)C4(CCCC4)c4ccccc4-5)cc32)c1]

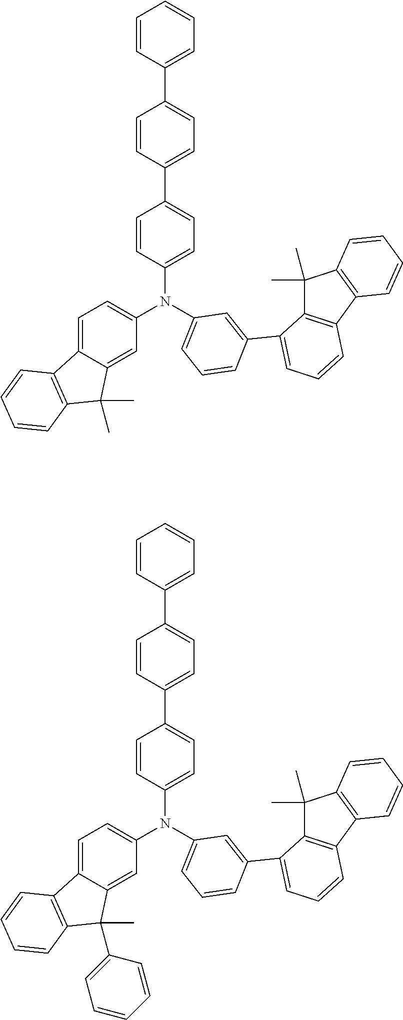 [SMILES: CC1(C)c2ccccc2-c2ccc(N(c3ccc(-c4ccc(-c5ccccc5)cc4)cc3)c3cccc(-c4cccc5c4C(C)(C)c4ccccc4-5)c3)cc21 ; CC1(C)c2ccccc2-c2cccc(-c3cccc(N(c4ccc(-c5ccc(-c6ccccc6)cc5)cc4)c4ccc5c(c4)C(C)(c4ccccc4)c4ccccc4-5)c3)c21]

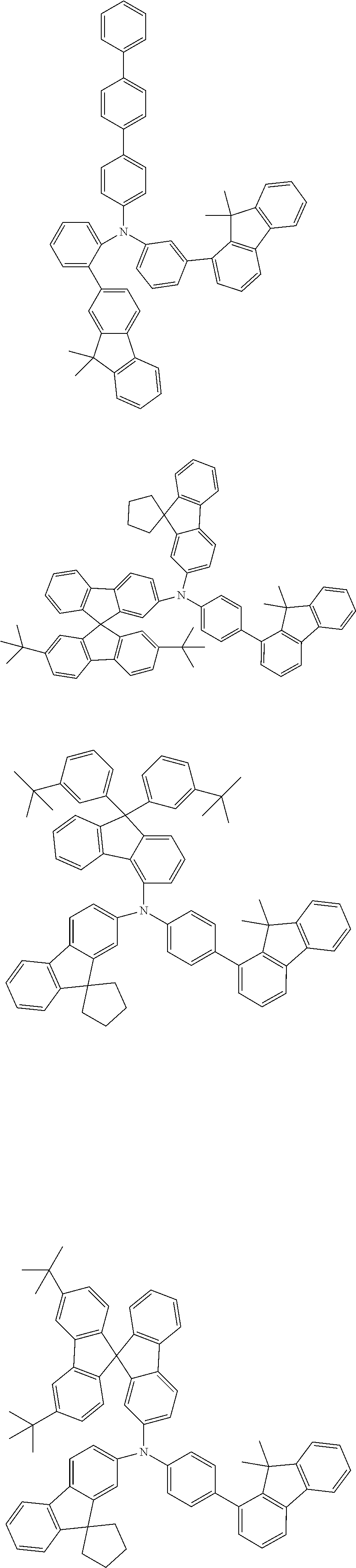

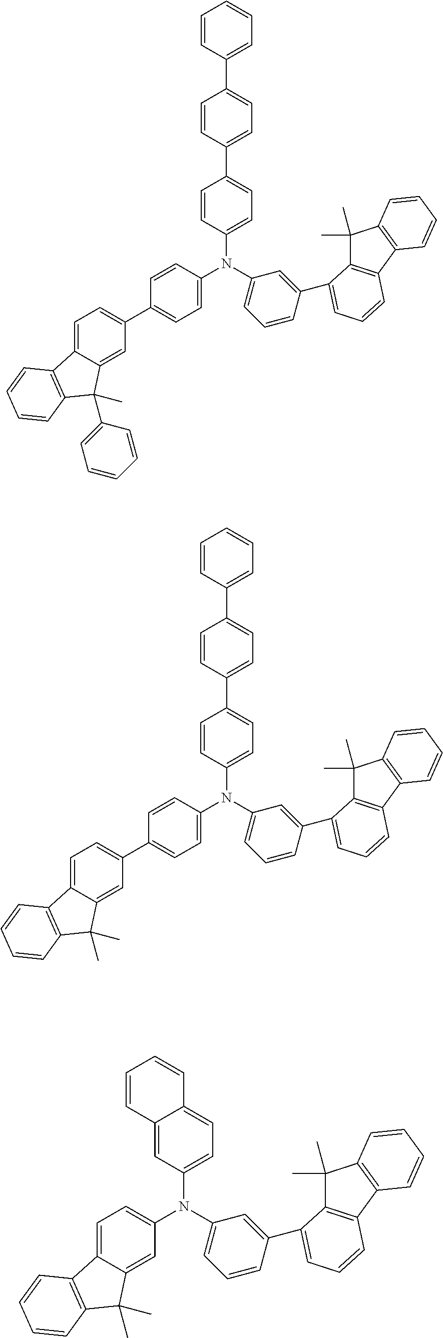 [SMILES: CC1(C)c2ccccc2-c2cccc(-c3cccc(N(c4ccc(-c5ccc(-c6ccccc6)cc5)cc4)c4ccc(-c5ccc6c(c5)C(C)(c5ccccc5)c5ccccc5-6)cc4)c3)c21 ; CC1(C)c2ccccc2-c2ccc(-c3ccc(N(c4ccc(-c5ccc(-c6ccccc6)cc5)cc4)c4cccc(-c5cccc6c5C(C)(C)c5ccccc5-6)c4)cc3)cc21 ; CC1(C)c2ccccc2-c2ccc(N(c3cccc(-c4cccc5c4C(C)(C)c4ccccc4-5)c3)c3ccc4ccccc4c3)cc21]

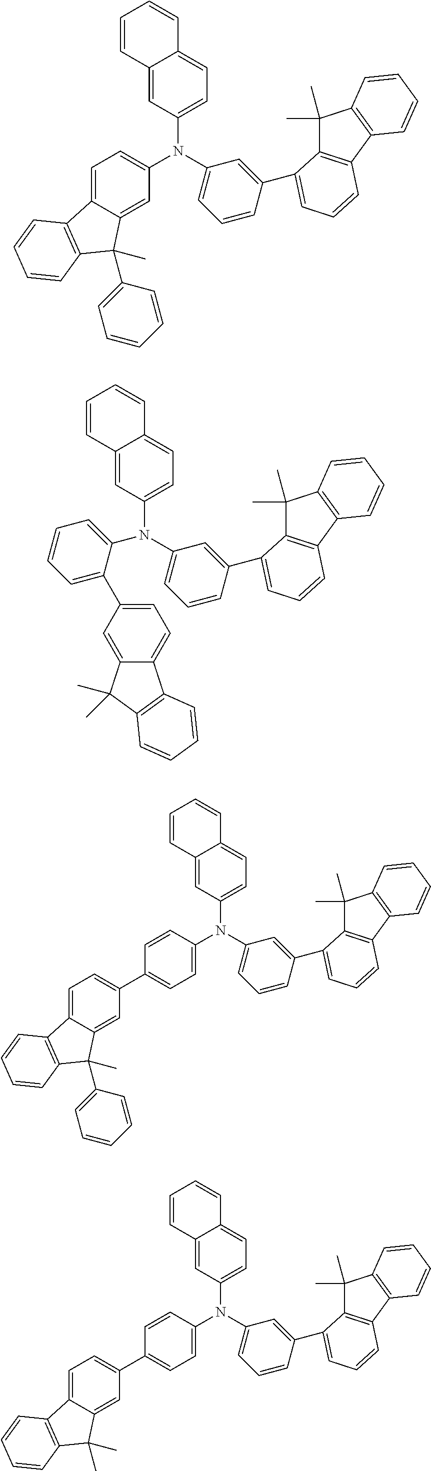 [SMILES: CC1(C)c2ccccc2-c2cccc(-c3cccc(N(c4ccc5c(c4)C(C)(c4ccccc4)c4ccccc4-5)c4ccc5ccccc5c4)c3)c21 ; CC1(C)c2ccccc2-c2ccc(-c3ccccc3N(c3cccc(-c4cccc5c4C(C)(C)c4ccccc4-5)c3)c3ccc4ccccc4c3)cc21 ; CC1(C)c2ccccc2-c2cccc(-c3cccc(N(c4ccc(-c5ccc6c(c5)C(C)(c5ccccc5)c5ccccc5-6)cc4)c4ccc5ccccc5c4)c3)c21 ; CC1(C)c2ccccc2-c2ccc(-c3ccc(N(c4cccc(-c5cccc6c5C(C)(C)c5ccccc5-6)c4)c4ccc5ccccc5c4)cc3)cc21]

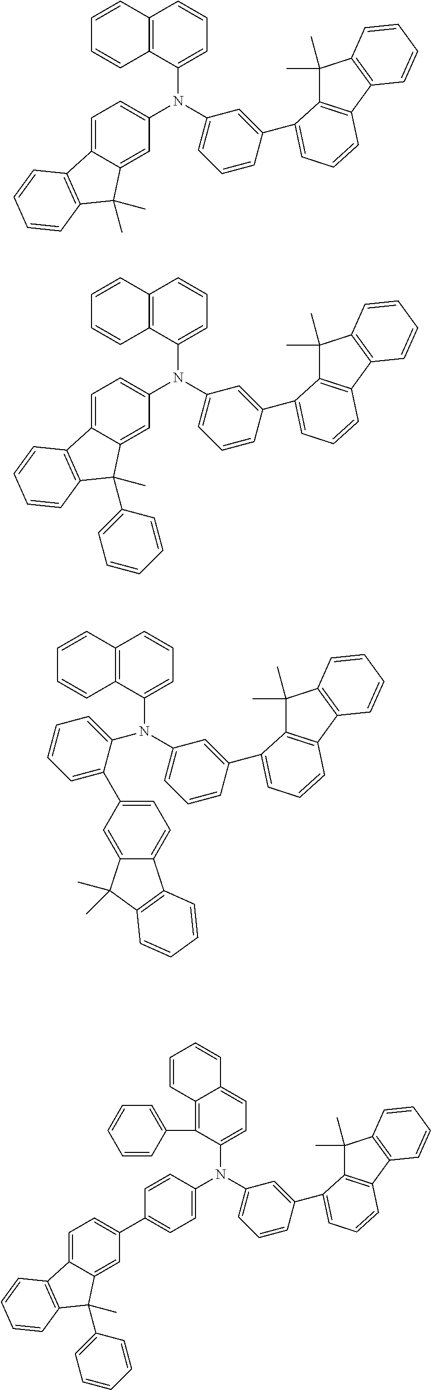

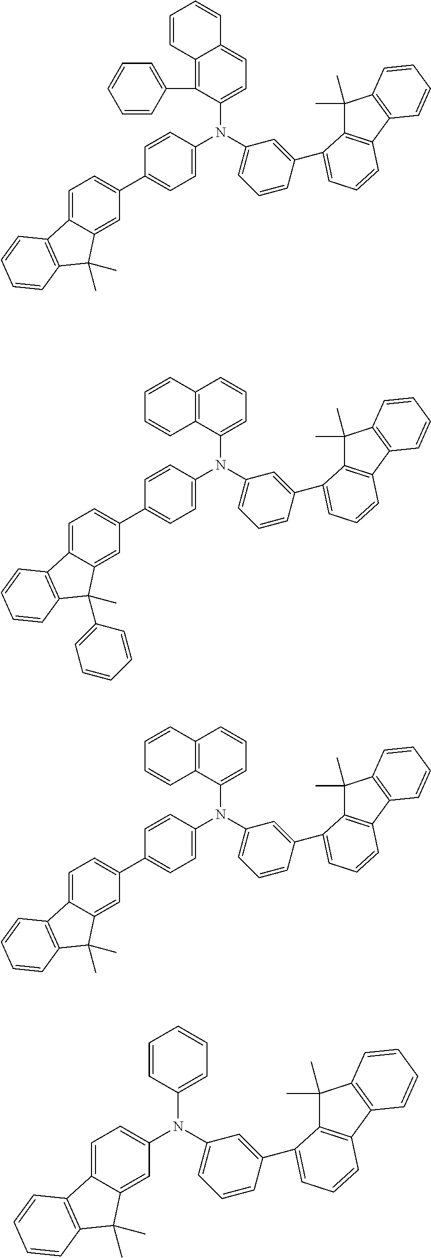 [SMILES: CC1(C)c2ccccc2-c2ccc(-c3ccc(N(c4cccc(-c5cccc6c5C(C)(C)c5ccccc5-6)c4)c4ccc5ccccc5c4-c4ccccc4)cc3)cc21 ; CC1(C)c2ccccc2-c2cccc(-c3cccc(N(c4ccc(-c5ccc6c(c5)C(C)(c5ccccc5)c5ccccc5-6)cc4)c4cccc5ccccc45)c3)c21 ; CC1(C)c2ccccc2-c2ccc(-c3ccc(N(c4cccc(-c5cccc6c5C(C)(C)c5ccccc5-6)c4)c4cccc5ccccc45)cc3)cc21 ; CC1(C)c2ccccc2-c2ccc(N(c3ccccc3)c3cccc(-c4cccc5c4C(C)(C)c4ccccc4-5)c3)cc21]

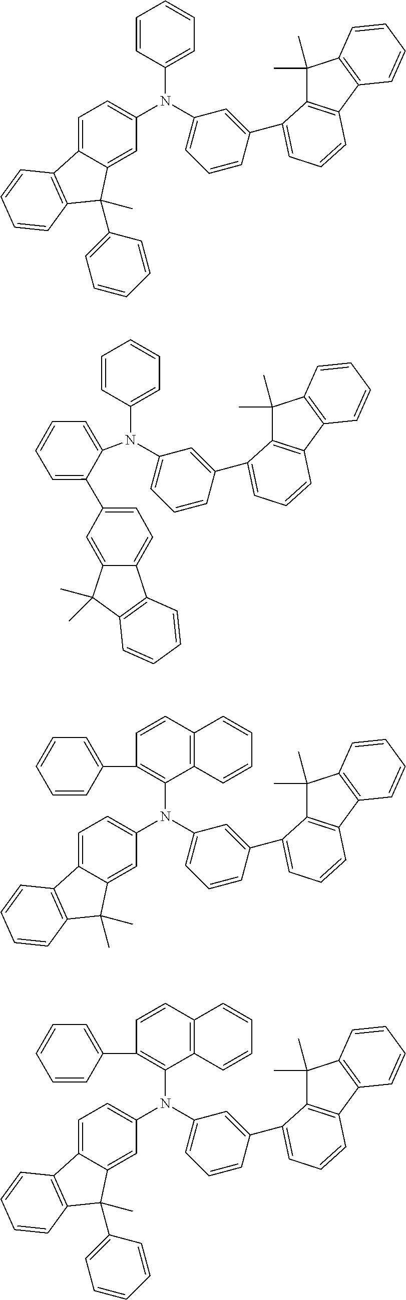

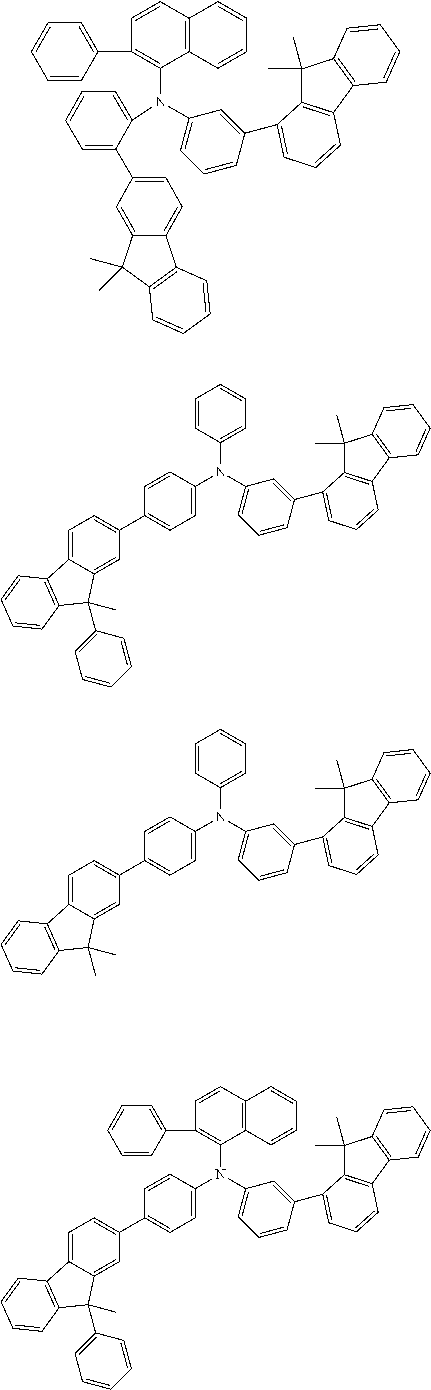 [SMILES: CC1(C)c2ccccc2-c2ccc(-c3ccccc3N(c3cccc(-c4cccc5c4C(C)(C)c4ccccc4-5)c3)c3c(-c4ccccc4)ccc4ccccc34)cc21 ; CC1(C)c2ccccc2-c2cccc(-c3cccc(N(c4ccccc4)c4ccc(-c5ccc6c(c5)C(C)(c5ccccc5)c5ccccc5-6)cc4)c3)c21 ; CC1(C)c2ccccc2-c2ccc(-c3ccc(N(c4ccccc4)c4cccc(-c5cccc6c5C(C)(C)c5ccccc5-6)c4)cc3)cc21 ; CC1(C)c2ccccc2-c2cccc(-c3cccc(N(c4ccc(-c5ccc6c(c5)C(C)(c5ccccc5)c5ccccc5-6)cc4)c4c(-c5ccccc5)ccc5ccccc45)c3)c21]

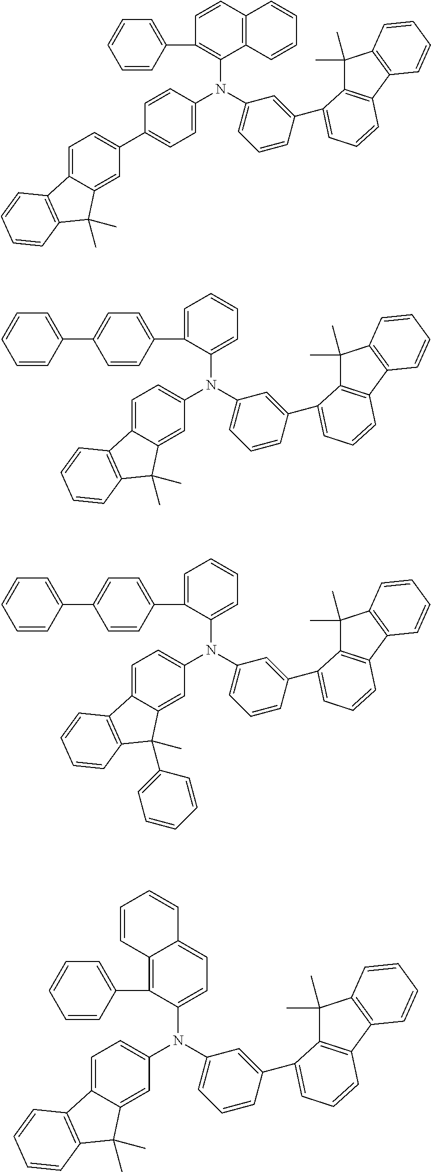 [SMILES: CC1(C)c2ccccc2-c2ccc(-c3ccc(N(c4cccc(-c5cccc6c5C(C)(C)c5ccccc5-6)c4)c4c(-c5ccccc5)ccc5ccccc45)cc3)cc21 ; CC1(C)c2ccccc2-c2ccc(N(c3cccc(-c4cccc5c4C(C)(C)c4ccccc4-5)c3)c3ccccc3-c3ccc(-c4ccccc4)cc3)cc21 ; CC1(C)c2ccccc2-c2cccc(-c3cccc(N(c4ccc5c(c4)C(C)(c4ccccc4)c4ccccc4-5)c4ccccc4-c4ccc(-c5ccccc5)cc4)c3)c21 ; CC1(C)c2ccccc2-c2ccc(N(c3cccc(-c4cccc5c4C(C)(C)c4ccccc4-5)c3)c3ccc4ccccc4c3-c3ccccc3)cc21]

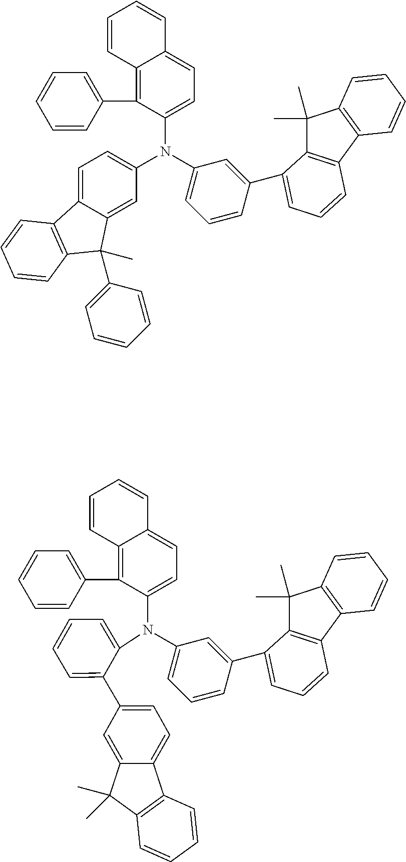 [SMILES: CC1(C)c2ccccc2-c2cccc(-c3cccc(N(c4ccc5c(c4)C(C)(c4ccccc4)c4ccccc4-5)c4ccc5ccccc5c4-c4ccccc4)c3)c21 ; CC1(C)c2ccccc2-c2ccc(-c3ccccc3N(c3cccc(-c4cccc5c4C(C)(C)c4ccccc4-5)c3)c3ccc4ccccc4c3-c3ccccc3)cc21]

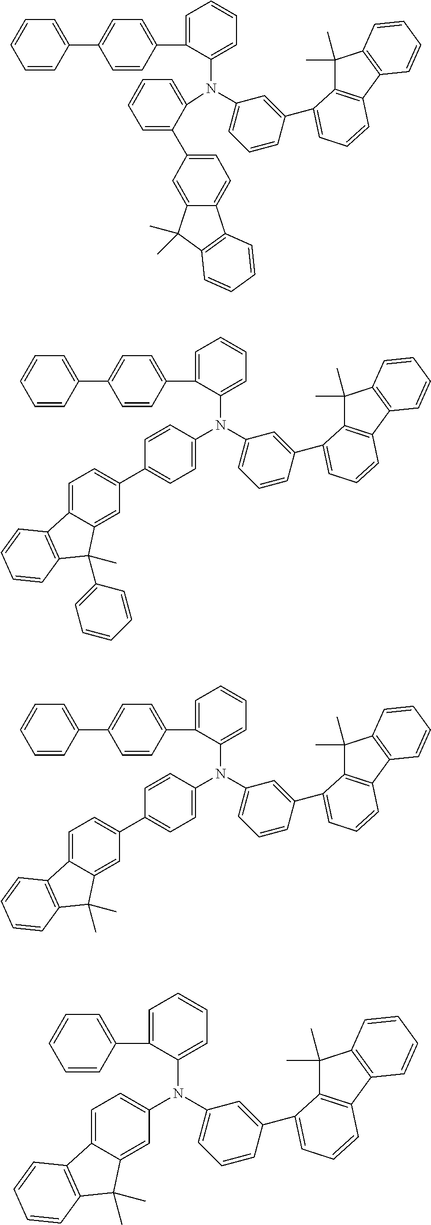 [SMILES: CC1(C)c2ccccc2-c2ccc(-c3ccccc3N(c3cccc(-c4cccc5c4C(C)(C)c4ccccc4-5)c3)c3ccccc3-c3ccc(-c4ccccc4)cc3)cc21 ; CC1(C)c2ccccc2-c2cccc(-c3cccc(N(c4ccc(-c5ccc6c(c5)C(C)(c5ccccc5)c5ccccc5-6)cc4)c4ccccc4-c4ccc(-c5ccccc5)cc4)c3)c21 ; CC1(C)c2ccccc2-c2ccc(-c3ccc(N(c4cccc(-c5cccc6c5C(C)(C)c5ccccc5-6)c4)c4ccccc4-c4ccc(-c5ccccc5)cc4)cc3)cc21 ; CC1(C)c2ccccc2-c2ccc(N(c3cccc(-c4cccc5c4C(C)(C)c4ccccc4-5)c3)c3ccccc3-c3ccccc3)cc21]

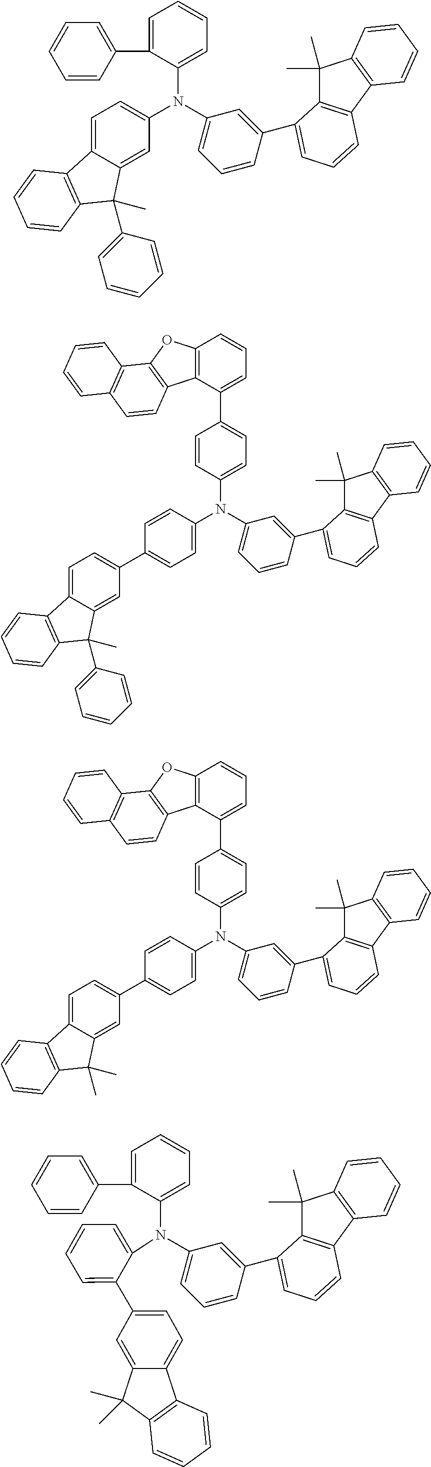 [SMILES: CC1(C)c2ccccc2-c2cccc(-c3cccc(N(c4ccc5c(c4)C(C)(c4ccccc4)c4ccccc4-5)c4ccccc4-c4ccccc4)c3)c21 ; CC1(C)c2ccccc2-c2cccc(-c3cccc(N(c4ccc(-c5ccc6c(c5)C(C)(c5ccccc5)c5ccccc5-6)cc4)c4ccc(-c5cccc6oc7c8ccccc8ccc7c56)cc4)c3)c21 ; CC1(C)c2ccccc2-c2ccc(-c3ccc(N(c4ccc(-c5cccc6oc7c8ccccc8ccc7c56)cc4)c4cccc(-c5cccc6c5C(C)(C)c5ccccc5-6)c4)cc3)cc21 ; CC1(C)c2ccccc2-c2ccc(-c3ccccc3N(c3cccc(-c4cccc5c4C(C)(C)c4ccccc4-5)c3)c3ccccc3-c3ccccc3)cc21]

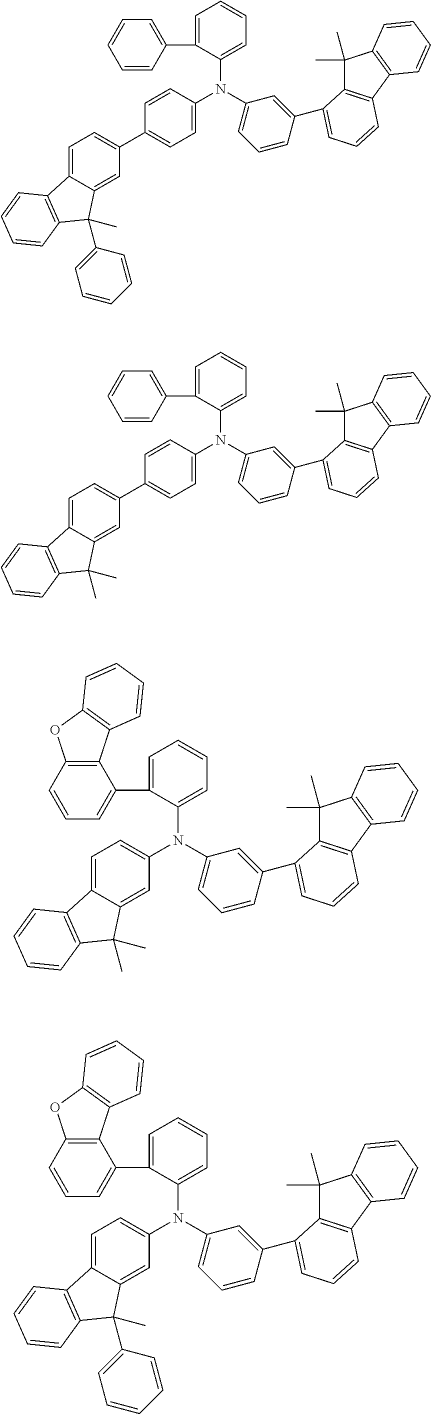 [SMILES: CC1(C)c2ccccc2-c2cccc(-c3cccc(N(c4ccc(-c5ccc6c(c5)C(C)(c5ccccc5)c5ccccc5-6)cc4)c4ccccc4-c4ccccc4)c3)c21 ; CC1(C)c2ccccc2-c2ccc(-c3ccc(N(c4cccc(-c5cccc6c5C(C)(C)c5ccccc5-6)c4)c4ccccc4-c4ccccc4)cc3)cc21 ; CC1(C)c2ccccc2-c2ccc(N(c3cccc(-c4cccc5c4C(C)(C)c4ccccc4-5)c3)c3ccccc3-c3cccc4oc5ccccc5c34)cc21 ; CC1(C)c2ccccc2-c2cccc(-c3cccc(N(c4ccc5c(c4)C(C)(c4ccccc4)c4ccccc4-5)c4ccccc4-c4cccc5oc6ccccc6c45)c3)c21]

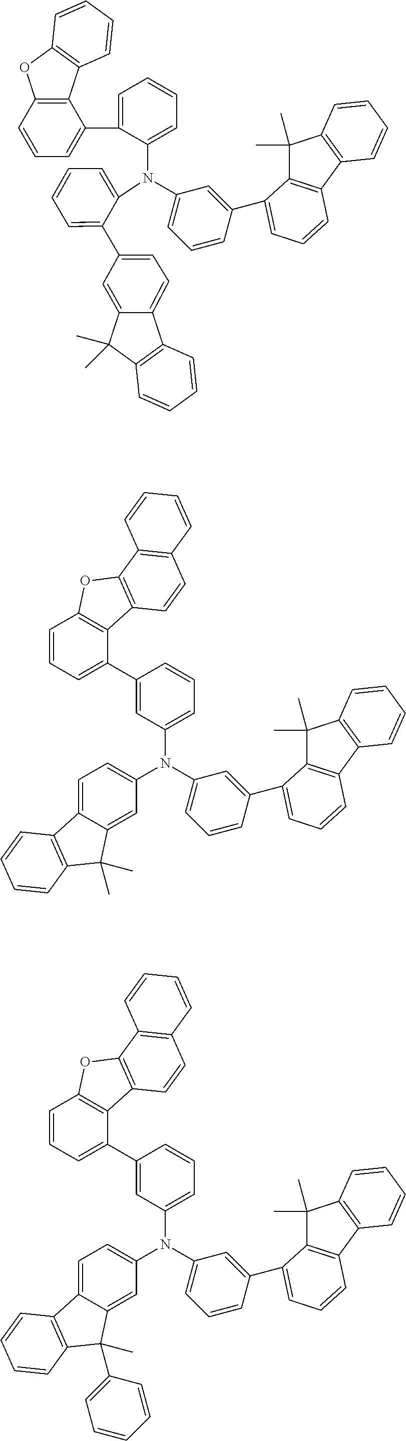 [SMILES: CC1(C)c2ccccc2-c2ccc(-c3ccccc3N(c3cccc(-c4cccc5c4C(C)(C)c4ccccc4-5)c3)c3ccccc3-c3cccc4oc5ccccc5c34)cc21 ; CC1(C)c2ccccc2-c2ccc(N(c3cccc(-c4cccc5c4C(C)(C)c4ccccc4-5)c3)c3cccc(-c4cccc5oc6c7ccccc7ccc6c45)c3)cc21 ; CC1(C)c2ccccc2-c2cccc(-c3cccc(N(c4cccc(-c5cccc6oc7c8ccccc8ccc7c56)c4)c4ccc5c(c4)C(C)(c4ccccc4)c4ccccc4-5)c3)c21]

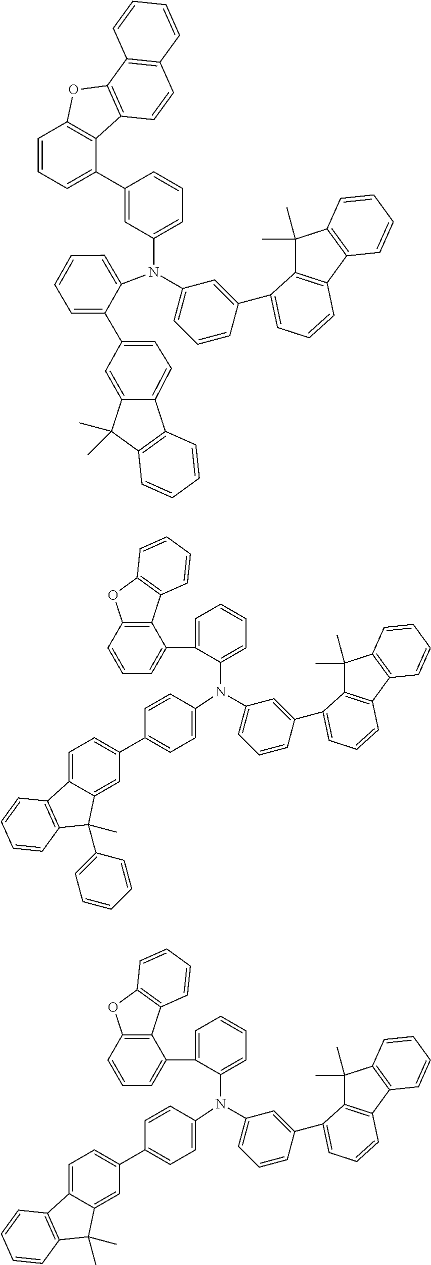

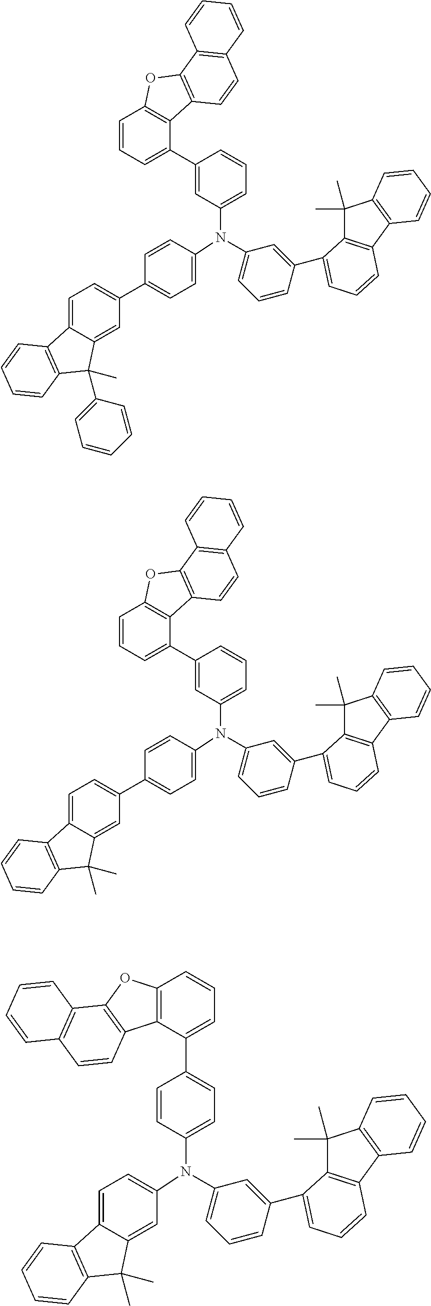 [SMILES: CC1(C)c2ccccc2-c2cccc(-c3cccc(N(c4ccc(-c5ccc6c(c5)C(C)(c5ccccc5)c5ccccc5-6)cc4)c4cccc(-c5cccc6oc7c8ccccc8ccc7c56)c4)c3)c21 ; CC1(C)c2ccccc2-c2ccc(-c3ccc(N(c4cccc(-c5cccc6c5C(C)(C)c5ccccc5-6)c4)c4cccc(-c5cccc6oc7c8ccccc8ccc7c56)c4)cc3)cc21 ; CC1(C)c2ccccc2-c2ccc(N(c3ccc(-c4cccc5oc6c7ccccc7ccc6c45)cc3)c3cccc(-c4cccc5c4C(C)(C)c4ccccc4-5)c3)cc21]

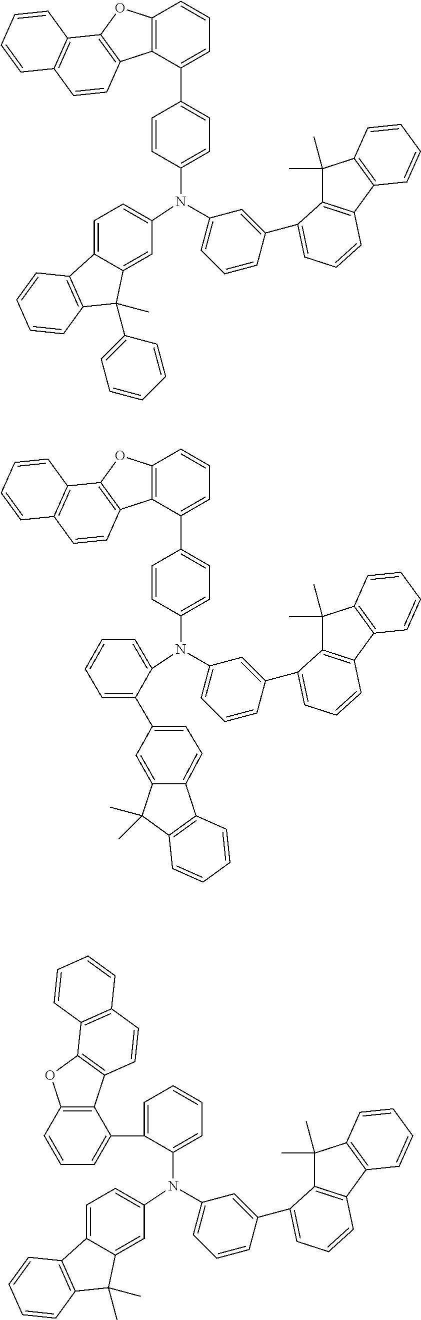 [SMILES: CC1(C)c2ccccc2-c2cccc(-c3cccc(N(c4ccc(-c5cccc6oc7c8ccccc8ccc7c56)cc4)c4ccc5c(c4)C(C)(c4ccccc4)c4ccccc4-5)c3)c21 ; CC1(C)c2ccccc2-c2ccc(-c3ccccc3N(c3ccc(-c4cccc5oc6c7ccccc7ccc6c45)cc3)c3cccc(-c4cccc5c4C(C)(C)c4ccccc4-5)c3)cc21 ; CC1(C)c2ccccc2-c2ccc(N(c3cccc(-c4cccc5c4C(C)(C)c4ccccc4-5)c3)c3ccccc3-c3cccc4oc5c6ccccc6ccc5c34)cc21]

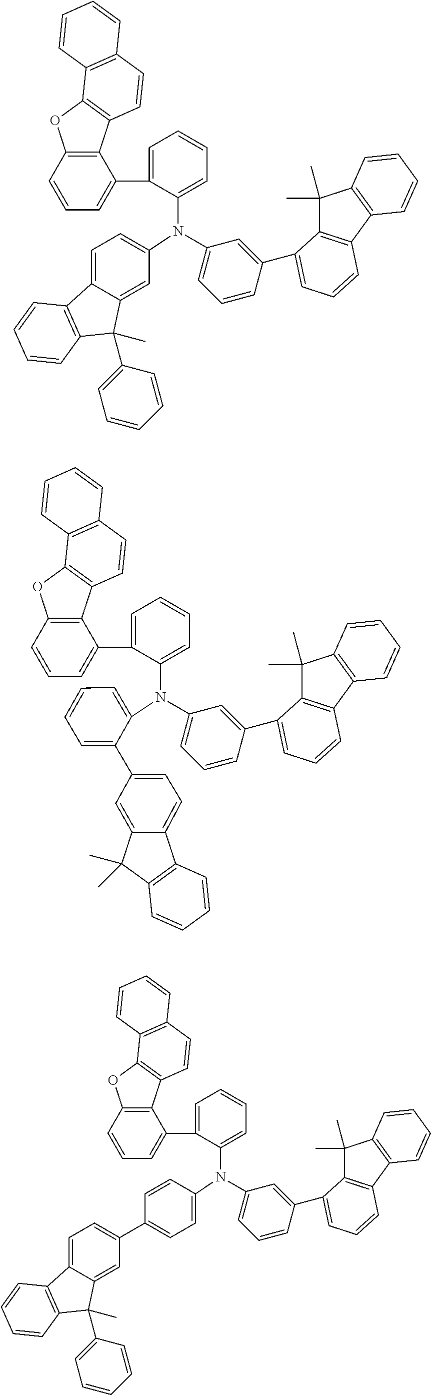 [SMILES: CC1(C)c2ccccc2-c2cccc(-c3cccc(N(c4ccc5c(c4)C(C)(c4ccccc4)c4ccccc4-5)c4ccccc4-c4cccc5oc6c7ccccc7ccc6c45)c3)c21 ; CC1(C)c2ccccc2-c2ccc(-c3ccccc3N(c3cccc(-c4cccc5c4C(C)(C)c4ccccc4-5)c3)c3ccccc3-c3cccc4oc5c6ccccc6ccc5c34)cc21 ; CC1(C)c2ccccc2-c2cccc(-c3cccc(N(c4ccc(-c5ccc6c(c5)C(C)(c5ccccc5)c5ccccc5-6)cc4)c4ccccc4-c4cccc5oc6c7ccccc7ccc6c45)c3)c21]

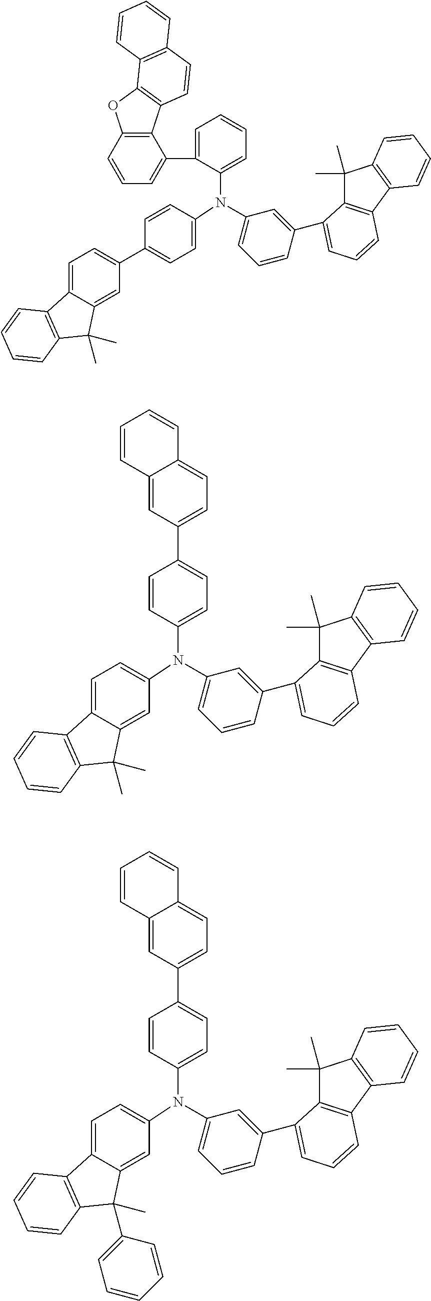 [SMILES: CC1(C)c2ccccc2-c2ccc(-c3ccc(N(c4cccc(-c5cccc6c5C(C)(C)c5ccccc5-6)c4)c4ccccc4-c4cccc5oc6c7ccccc7ccc6c45)cc3)cc21 ; CC1(C)c2ccccc2-c2ccc(N(c3ccc(-c4ccc5ccccc5c4)cc3)c3cccc(-c4cccc5c4C(C)(C)c4ccccc4-5)c3)cc21 ; CC1(C)c2ccccc2-c2cccc(-c3cccc(N(c4ccc(-c5ccc6ccccc6c5)cc4)c4ccc5c(c4)C(C)(c4ccccc4)c4ccccc4-5)c3)c21]

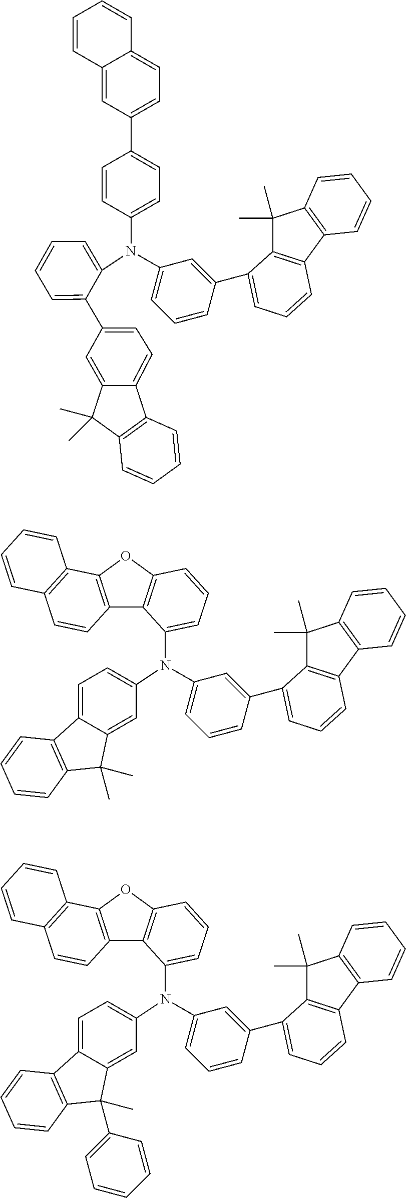 [SMILES: CC1(C)c2ccccc2-c2ccc(-c3ccccc3N(c3ccc(-c4ccc5ccccc5c4)cc3)c3cccc(-c4cccc5c4C(C)(C)c4ccccc4-5)c3)cc21 ; CC1(C)c2ccccc2-c2ccc(N(c3cccc(-c4cccc5c4C(C)(C)c4ccccc4-5)c3)c3cccc4oc5c6ccccc6ccc5c34)cc21 ; CC1(C)c2ccccc2-c2cccc(-c3cccc(N(c4ccc5c(c4)C(C)(c4ccccc4)c4ccccc4-5)c4cccc5oc6c7ccccc7ccc6c45)c3)c21]

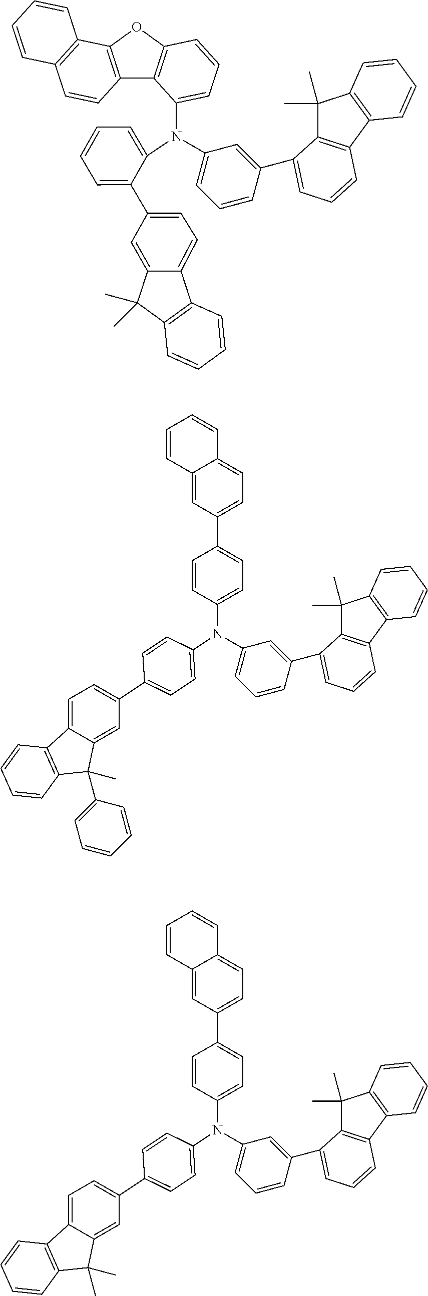 [SMILES: CC1(C)c2ccccc2-c2ccc(-c3ccccc3N(c3cccc(-c4cccc5c4C(C)(C)c4ccccc4-5)c3)c3cccc4oc5c6ccccc6ccc5c34)cc21 ; CC1(C)c2ccccc2-c2cccc(-c3cccc(N(c4ccc(-c5ccc6c(c5)C(C)(c5ccccc5)c5ccccc5-6)cc4)c4ccc(-c5ccc6ccccc6c5)cc4)c3)c21 ; CC1(C)c2ccccc2-c2ccc(-c3ccc(N(c4ccc(-c5ccc6ccccc6c5)cc4)c4cccc(-c5cccc6c5C(C)(C)c5ccccc5-6)c4)cc3)cc21]

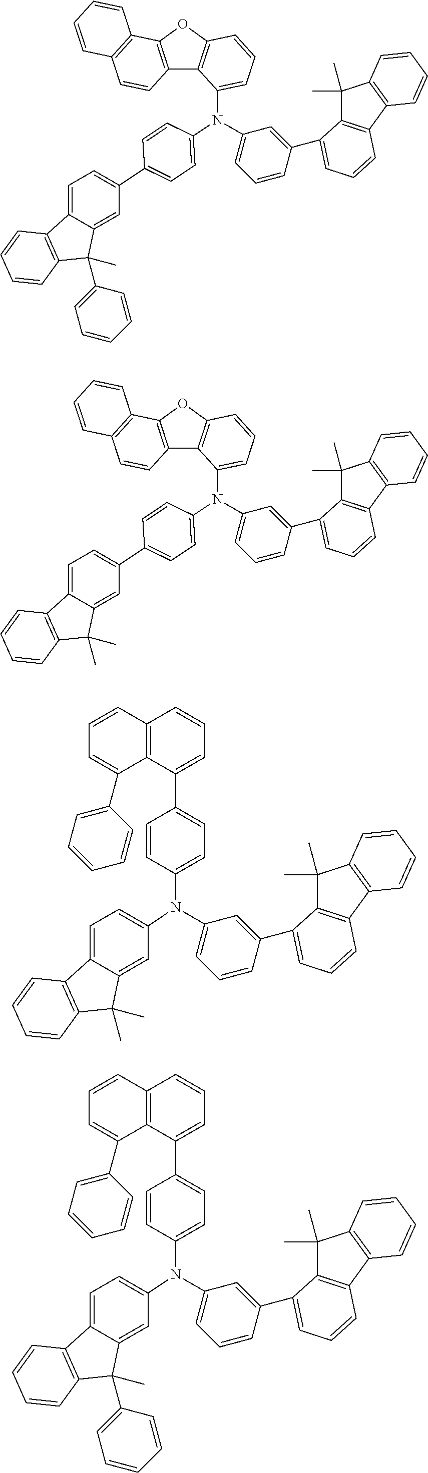

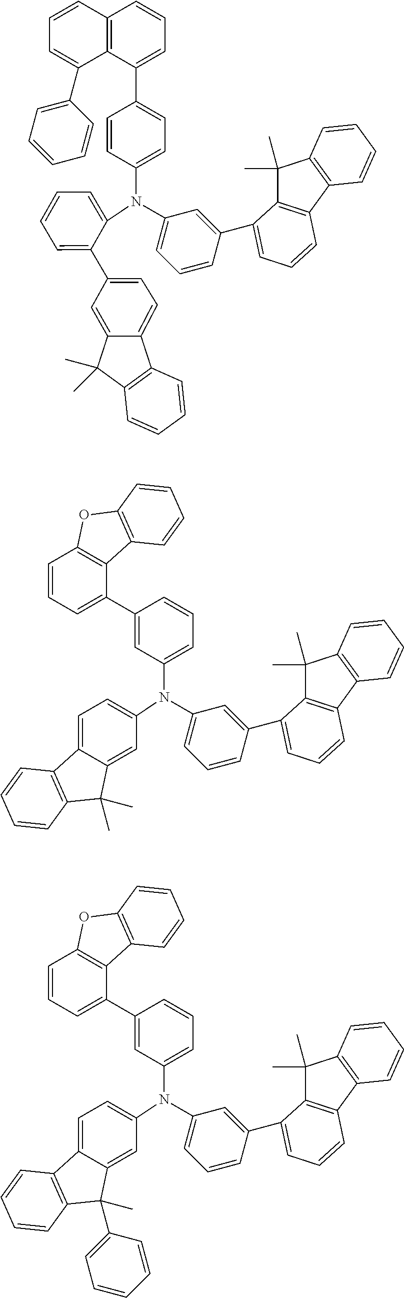 [SMILES: CC1(C)c2ccccc2-c2ccc(-c3ccccc3N(c3ccc(-c4cccc5cccc(-c6ccccc6)c45)cc3)c3cccc(-c4cccc5c4C(C)(C)c4ccccc4-5)c3)cc21 ; CC1(C)c2ccccc2-c2ccc(N(c3cccc(-c4cccc5c4C(C)(C)c4ccccc4-5)c3)c3cccc(-c4cccc5oc6ccccc6c45)c3)cc21 ; CC1(C)c2ccccc2-c2cccc(-c3cccc(N(c4cccc(-c5cccc6oc7ccccc7c56)c4)c4ccc5c(c4)C(C)(c4ccccc4)c4ccccc4-5)c3)c21]

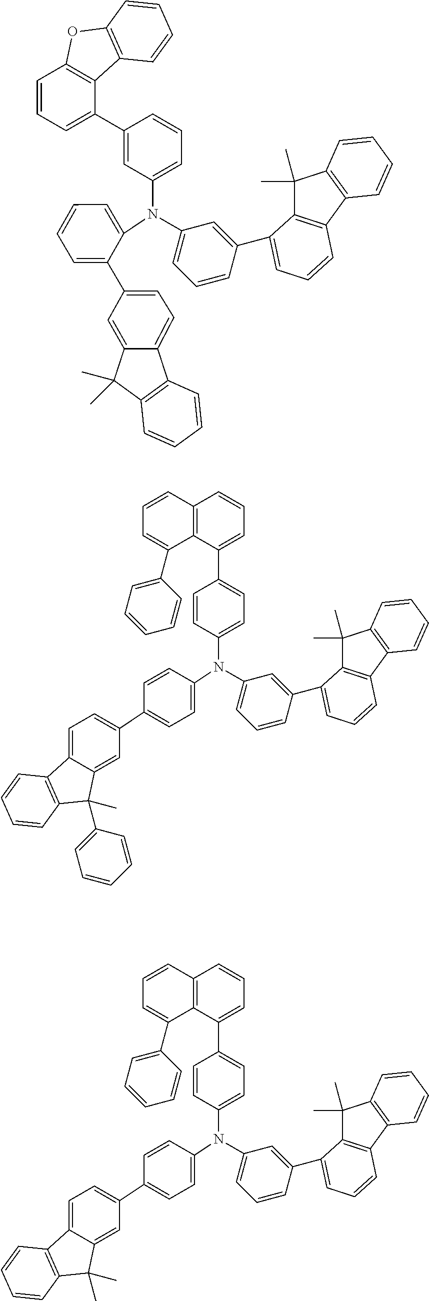 [SMILES: CC1(C)c2ccccc2-c2ccc(-c3ccccc3N(c3cccc(-c4cccc5c4C(C)(C)c4ccccc4-5)c3)c3cccc(-c4cccc5oc6ccccc6c45)c3)cc21 ; CC1(C)c2ccccc2-c2cccc(-c3cccc(N(c4ccc(-c5ccc6c(c5)C(C)(c5ccccc5)c5ccccc5-6)cc4)c4ccc(-c5cccc6cccc(-c7ccccc7)c56)cc4)c3)c21 ; CC1(C)c2ccccc2-c2ccc(-c3ccc(N(c4ccc(-c5cccc6cccc(-c7ccccc7)c56)cc4)c4cccc(-c5cccc6c5C(C)(C)c5ccccc5-6)c4)cc3)cc21]

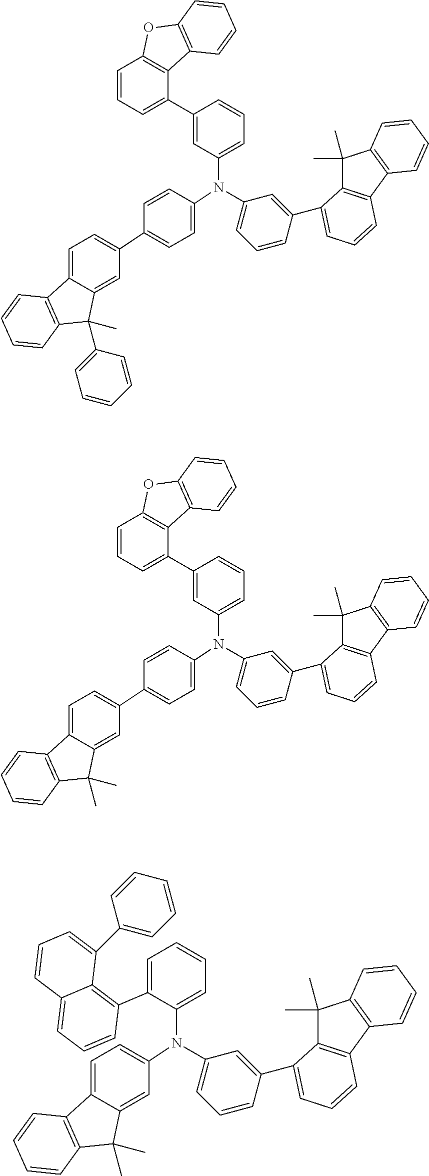 [SMILES: CC1(C)c2ccccc2-c2cccc(-c3cccc(N(c4ccc(-c5ccc6c(c5)C(C)(c5ccccc5)c5ccccc5-6)cc4)c4cccc(-c5cccc6oc7ccccc7c56)c4)c3)c21 ; CC1(C)c2ccccc2-c2ccc(-c3ccc(N(c4cccc(-c5cccc6c5C(C)(C)c5ccccc5-6)c4)c4cccc(-c5cccc6oc7ccccc7c56)c4)cc3)cc21 ; CC1(C)c2ccccc2-c2ccc(N(c3cccc(-c4cccc5c4C(C)(C)c4ccccc4-5)c3)c3ccccc3-c3cccc4cccc(-c5ccccc5)c34)cc21]

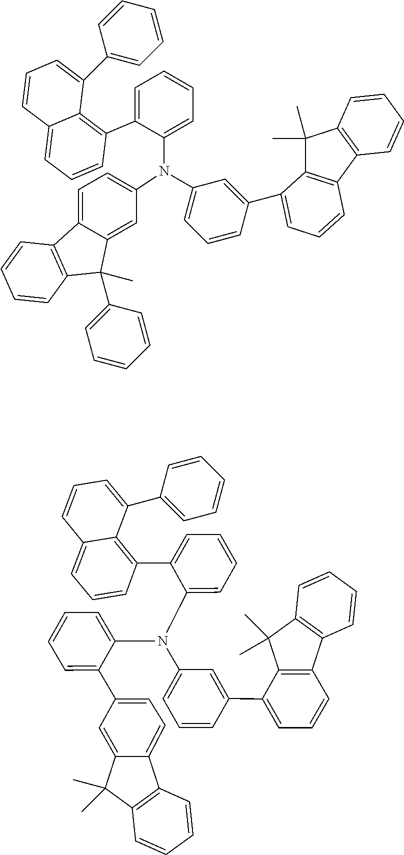 [SMILES: CC1(C)c2ccccc2-c2cccc(-c3cccc(N(c4ccc5c(c4)C(C)(c4ccccc4)c4ccccc4-5)c4ccccc4-c4cccc5cccc(-c6ccccc6)c45)c3)c21 ; CC1(C)c2ccccc2-c2ccc(-c3ccccc3N(c3cccc(-c4cccc5c4C(C)(C)c4ccccc4-5)c3)c3ccccc3-c3cccc4cccc(-c5ccccc5)c34)cc21]

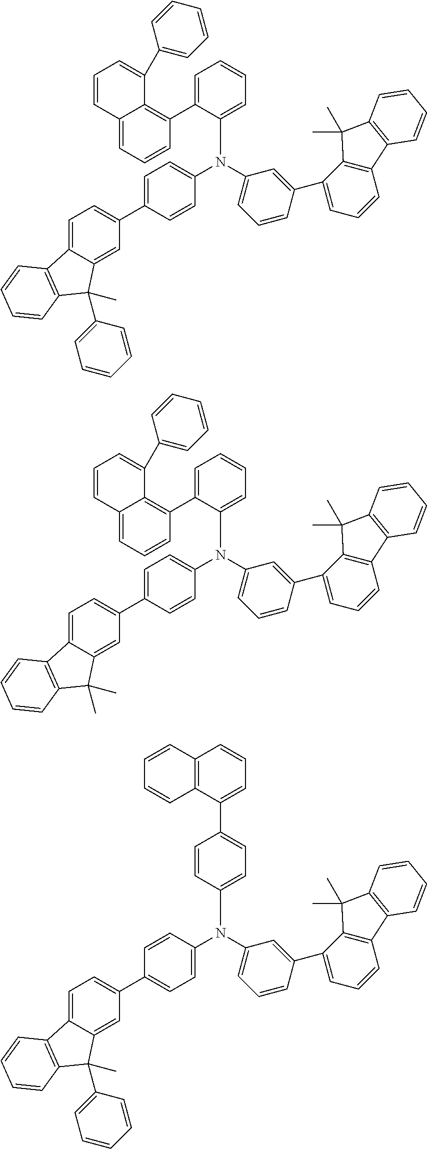 [SMILES: CC1(C)c2ccccc2-c2cccc(-c3cccc(N(c4ccc(-c5ccc6c(c5)C(C)(c5ccccc5)c5ccccc5-6)cc4)c4ccccc4-c4cccc5cccc(-c6ccccc6)c45)c3)c21 ; CC1(C)c2ccccc2-c2ccc(-c3ccc(N(c4cccc(-c5cccc6c5C(C)(C)c5ccccc5-6)c4)c4ccccc4-c4cccc5cccc(-c6ccccc6)c45)cc3)cc21 ; CC1(C)c2ccccc2-c2cccc(-c3cccc(N(c4ccc(-c5ccc6c(c5)C(C)(c5ccccc5)c5ccccc5-6)cc4)c4ccc(-c5cccc6ccccc56)cc4)c3)c21]

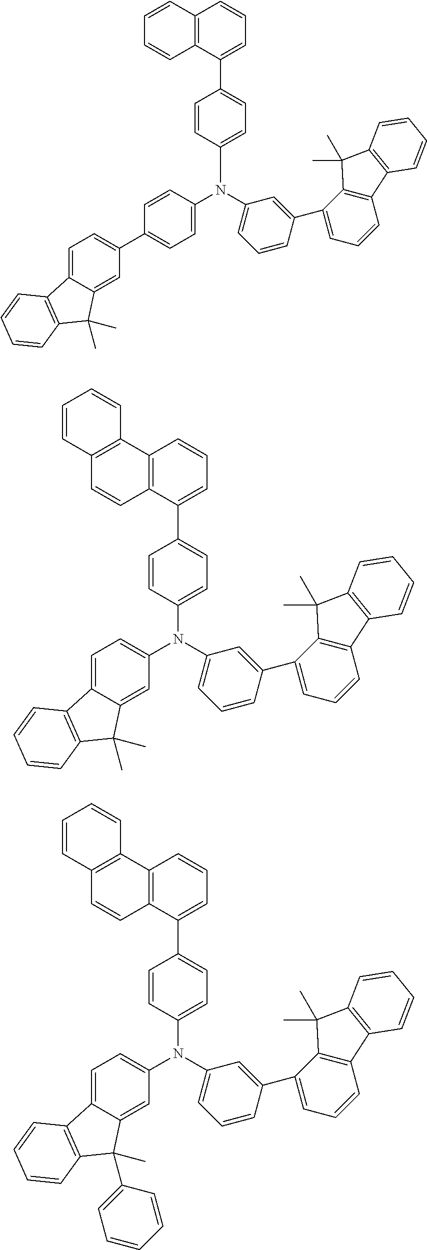

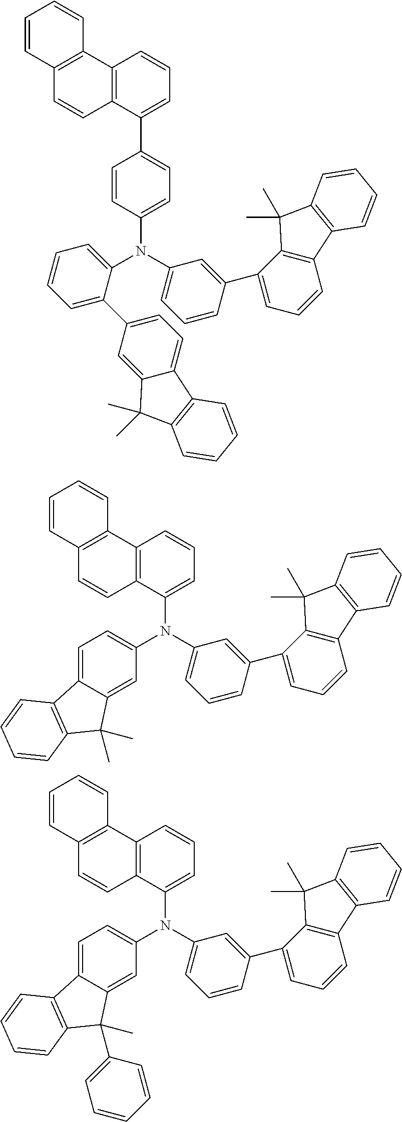

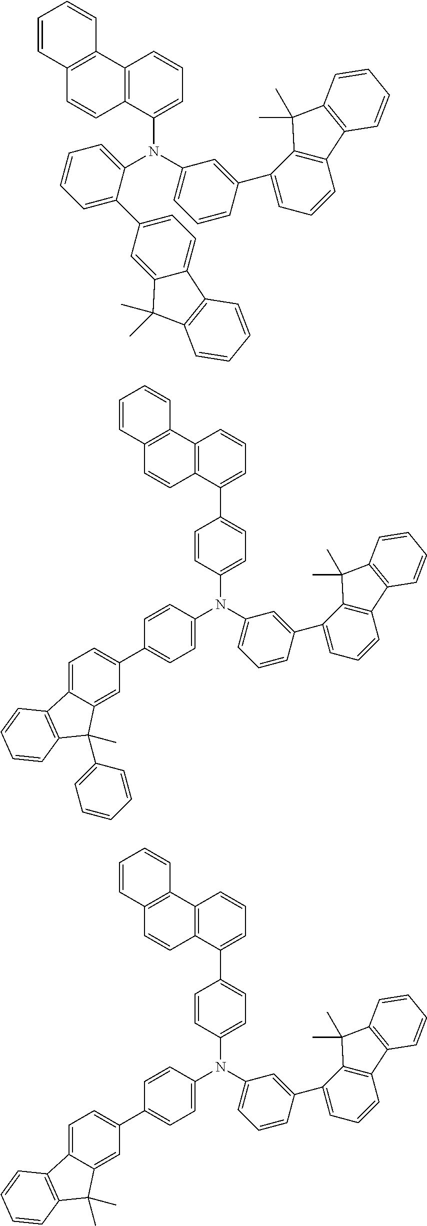 [SMILES: CC1(C)c2ccccc2-c2ccc(-c3ccccc3N(c3cccc(-c4cccc5c4C(C)(C)c4ccccc4-5)c3)c3cccc4c3ccc3ccccc34)cc21 ; CC1(C)c2ccccc2-c2cccc(-c3cccc(N(c4ccc(-c5ccc6c(c5)C(C)(c5ccccc5)c5ccccc5-6)cc4)c4ccc(-c5cccc6c5ccc5ccccc56)cc4)c3)c21 ; CC1(C)c2ccccc2-c2ccc(-c3ccc(N(c4ccc(-c5cccc6c5ccc5ccccc56)cc4)c4cccc(-c5cccc6c5C(C)(C)c5ccccc5-6)c4)cc3)cc21]

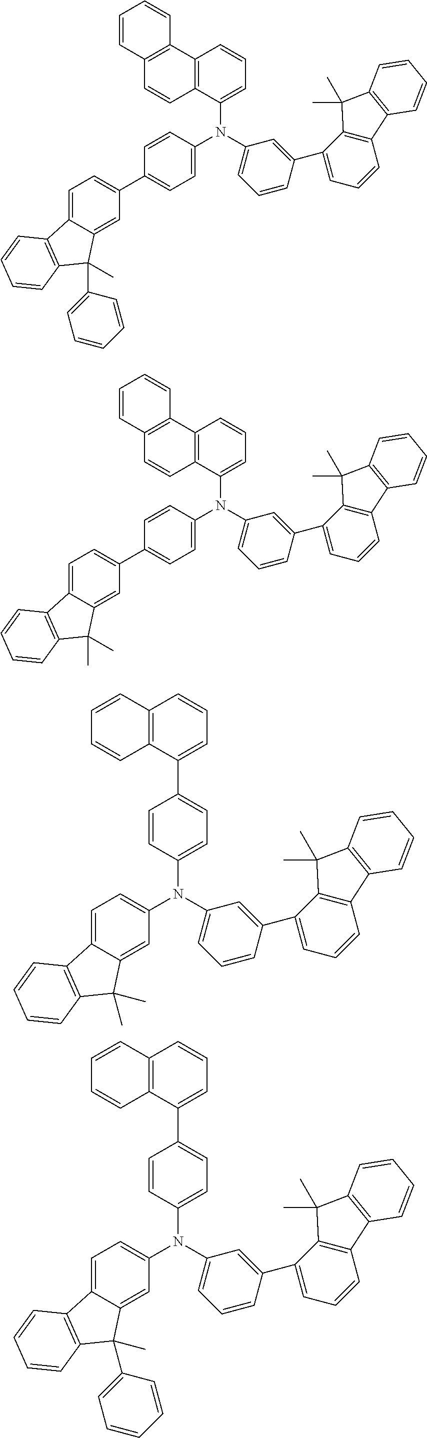

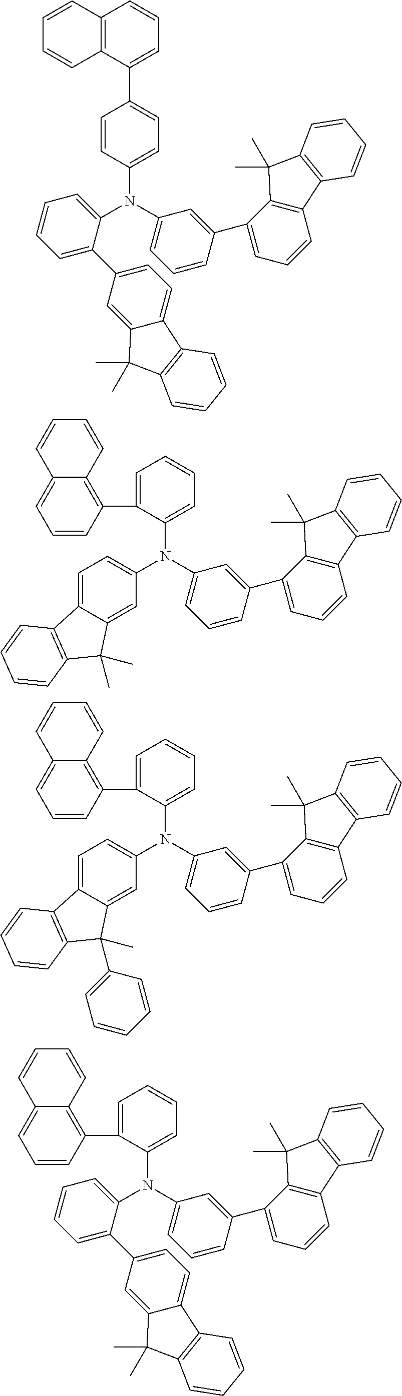

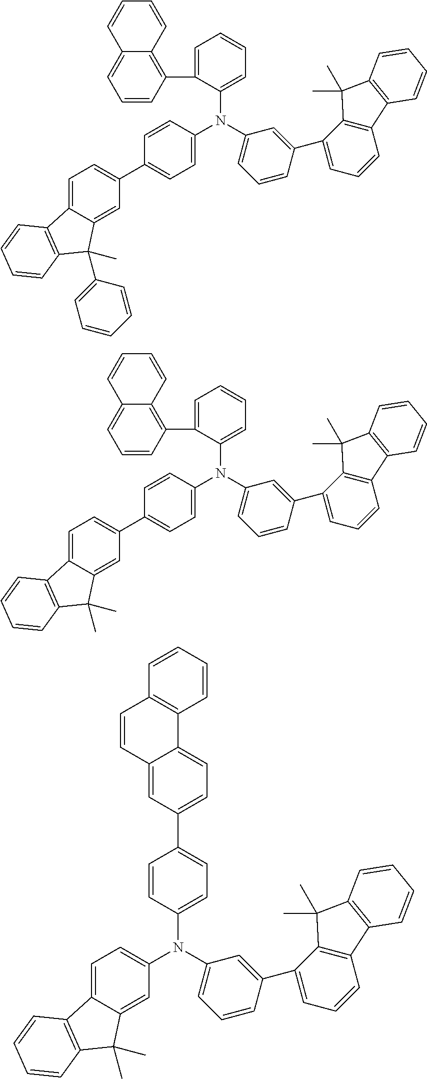

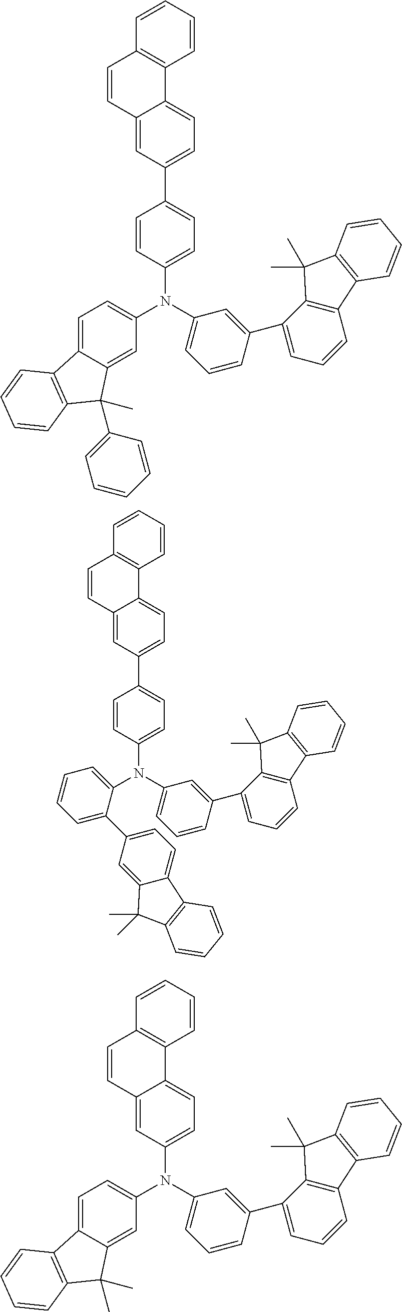

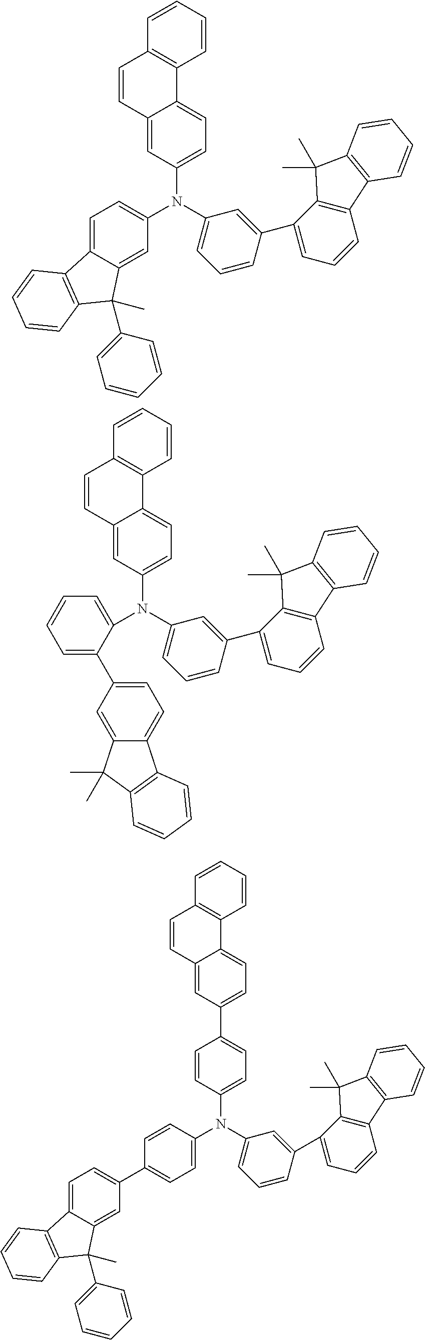

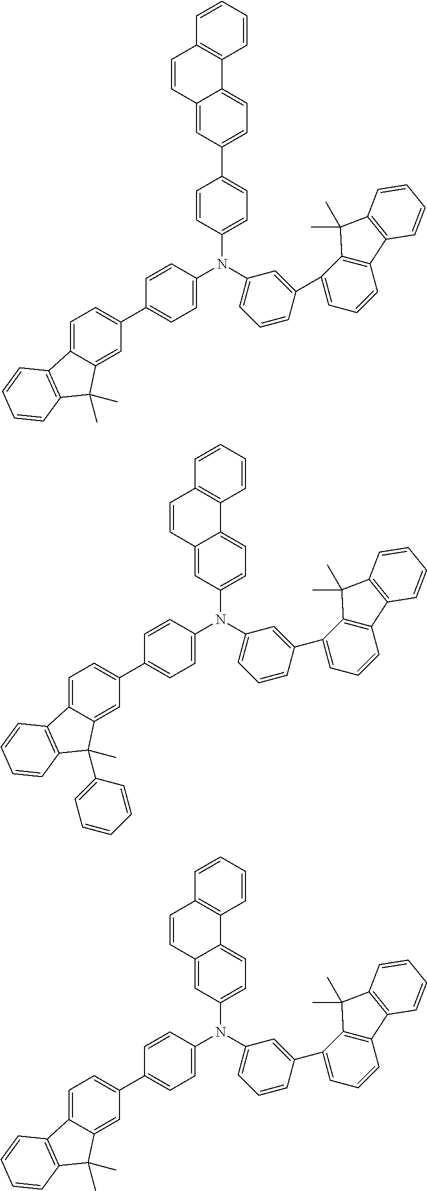 [SMILES: CC1(C)c2ccccc2-c2ccc(-c3ccc(N(c4ccc(-c5ccc6c(ccc7ccccc76)c5)cc4)c4cccc(-c5cccc6c5C(C)(C)c5ccccc5-6)c4)cc3)cc21 ; CC1(C)c2ccccc2-c2cccc(-c3cccc(N(c4ccc(-c5ccc6c(c5)C(C)(c5ccccc5)c5ccccc5-6)cc4)c4ccc5c(ccc6ccccc65)c4)c3)c21 ; CC1(C)c2ccccc2-c2ccc(-c3ccc(N(c4cccc(-c5cccc6c5C(C)(C)c5ccccc5-6)c4)c4ccc5c(ccc6ccccc65)c4)cc3)cc21]

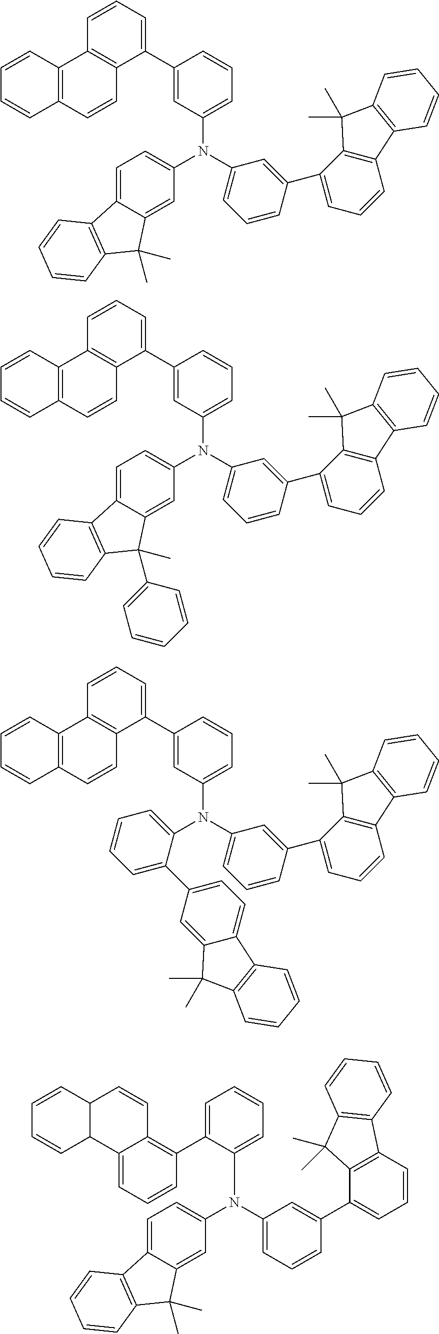

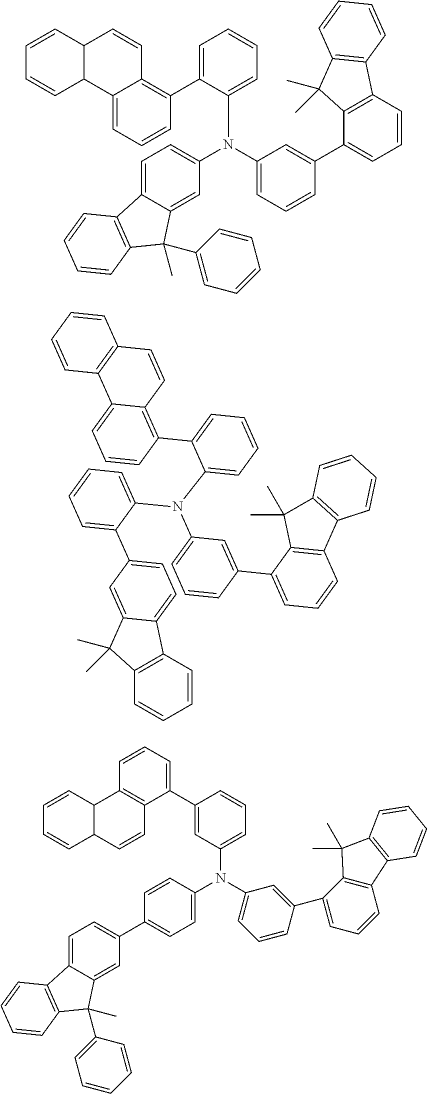

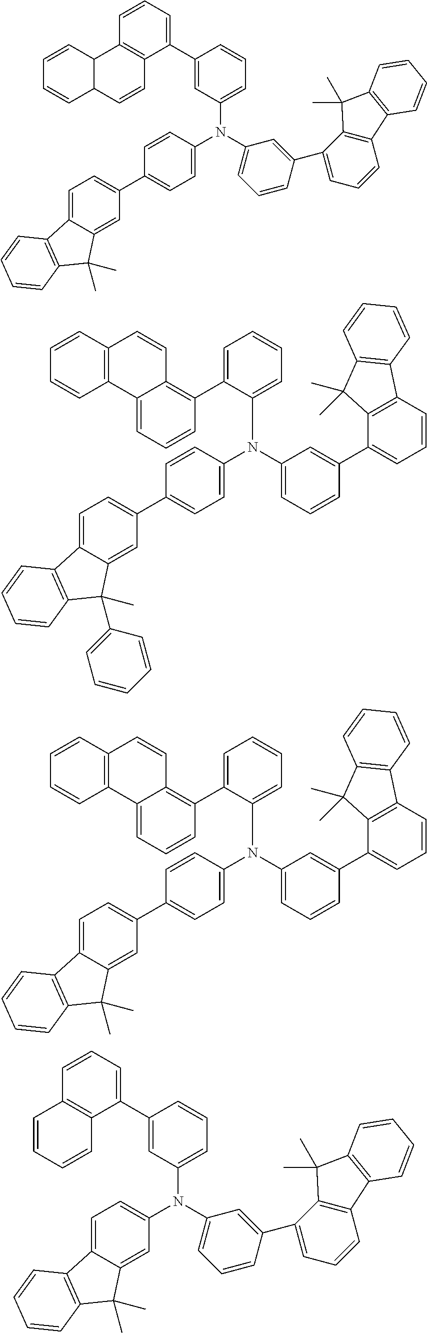

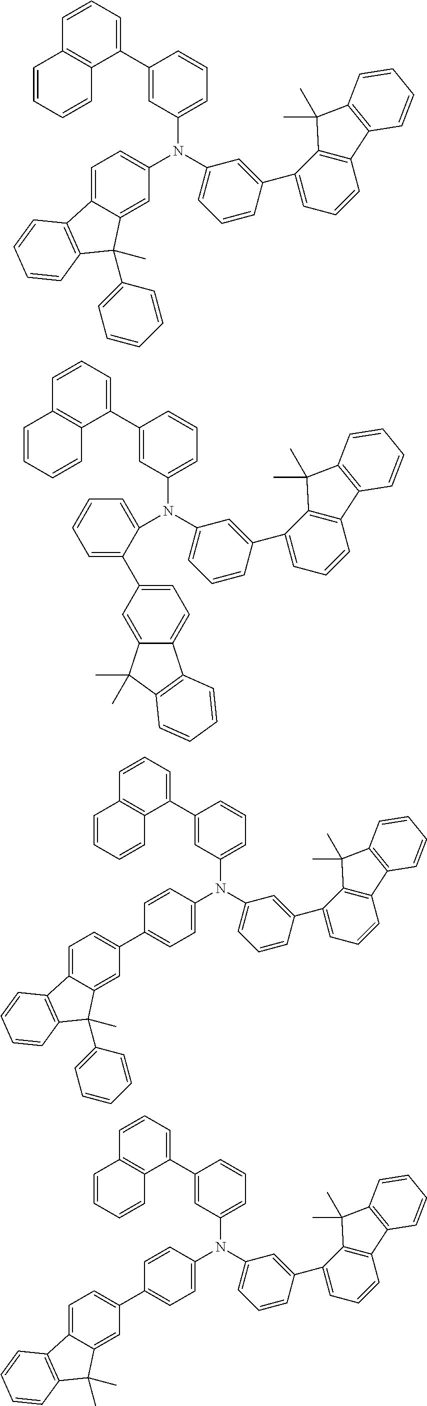

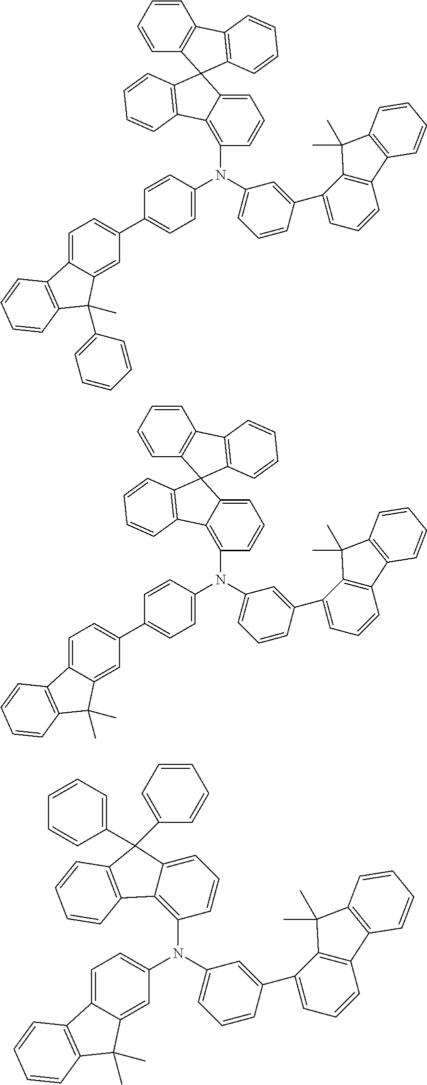 [SMILES: CC1(C)c2ccccc2-c2cccc(-c3cccc(N(c4ccc(-c5ccc6c(c5)C(C)(c5ccccc5)c5ccccc5-6)cc4)c4cccc5c4-c4ccccc4C54c5ccccc5-c5ccccc54)c3)c21 ; CC1(C)c2ccccc2-c2ccc(-c3ccc(N(c4cccc(-c5cccc6c5C(C)(C)c5ccccc5-6)c4)c4cccc5c4-c4ccccc4C54c5ccccc5-c5ccccc54)cc3)cc21 ; CC1(C)c2ccccc2-c2ccc(N(c3cccc(-c4cccc5c4C(C)(C)c4ccccc4-5)c3)c3cccc4c3-c3ccccc3C4(c3ccccc3)c3ccccc3)cc21]

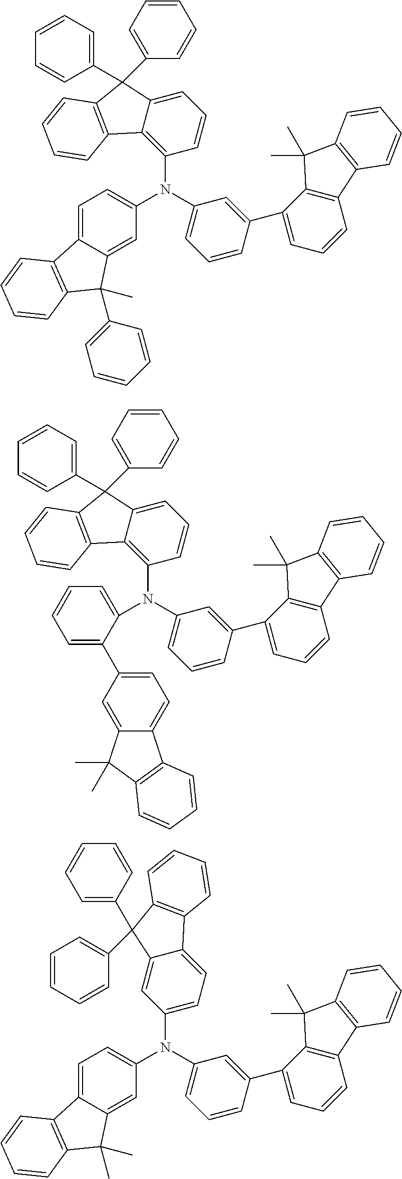

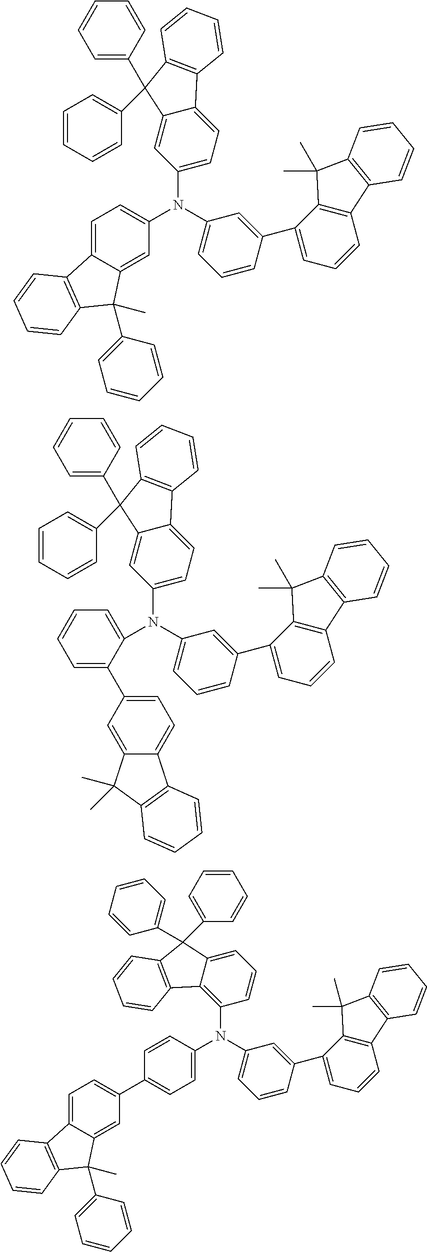

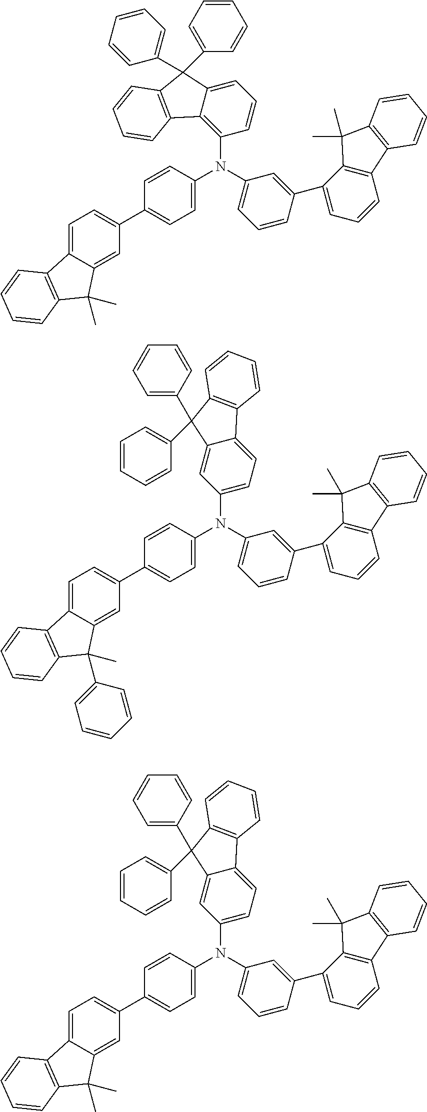 [SMILES: CC1(C)c2ccccc2-c2ccc(-c3ccc(N(c4cccc(-c5cccc6c5C(C)(C)c5ccccc5-6)c4)c4cccc5c4-c4ccccc4C5(c4ccccc4)c4ccccc4)cc3)cc21 ; CC1(C)c2ccccc2-c2cccc(-c3cccc(N(c4ccc(-c5ccc6c(c5)C(C)(c5ccccc5)c5ccccc5-6)cc4)c4ccc5c(c4)C(c4ccccc4)(c4ccccc4)c4ccccc4-5)c3)c21 ; CC1(C)c2ccccc2-c2ccc(-c3ccc(N(c4cccc(-c5cccc6c5C(C)(C)c5ccccc5-6)c4)c4ccc5c(c4)C(c4ccccc4)(c4ccccc4)c4ccccc4-5)cc3)cc21]

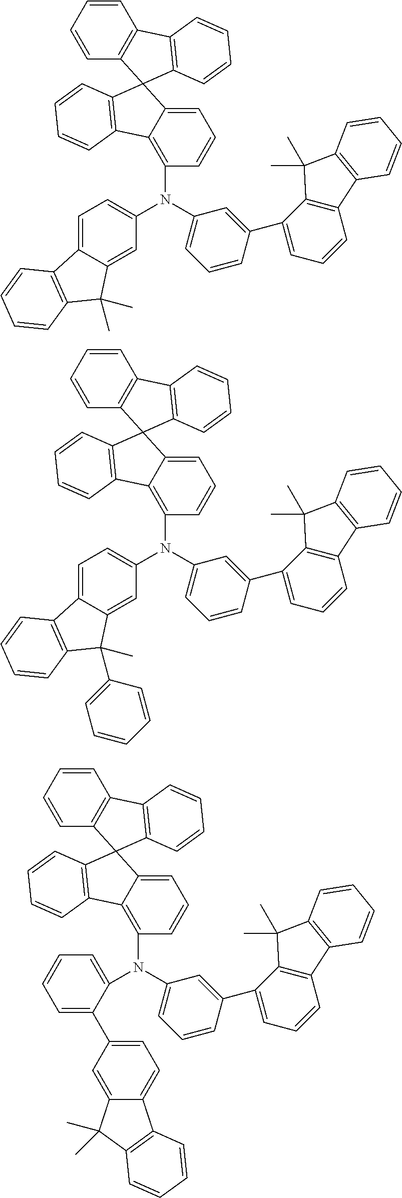

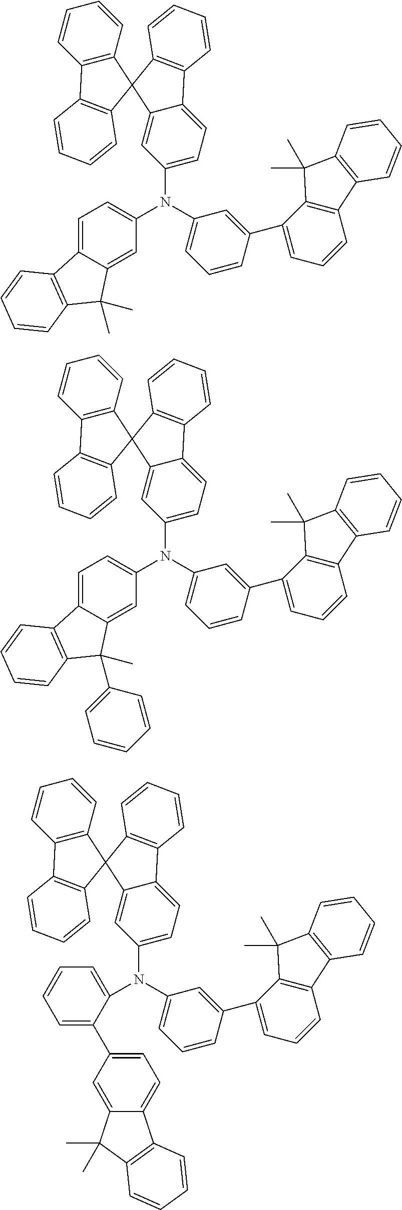

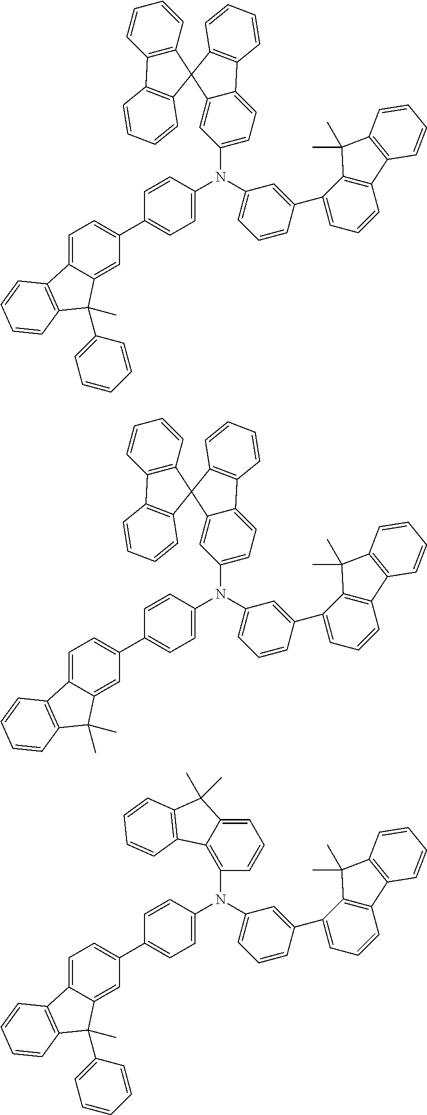 [SMILES: CC1(C)c2ccccc2-c2cccc(-c3cccc(N(c4ccc(-c5ccc6c(c5)C(C)(c5ccccc5)c5ccccc5-6)cc4)c4ccc5c(c4)C4(c6ccccc6-c6ccccc64)c4ccccc4-5)c3)c21 ; CC1(C)c2ccccc2-c2ccc(-c3ccc(N(c4cccc(-c5cccc6c5C(C)(C)c5ccccc5-6)c4)c4ccc5c(c4)C4(c6ccccc6-c6ccccc64)c4ccccc4-5)cc3)cc21 ; CC1(C)c2ccccc2-c2c(N(c3ccc(-c4ccc5c(c4)C(C)(c4ccccc4)c4ccccc4-5)cc3)c3cccc(-c4cccc5c4C(C)(C)c4ccccc4-5)c3)cccc21]

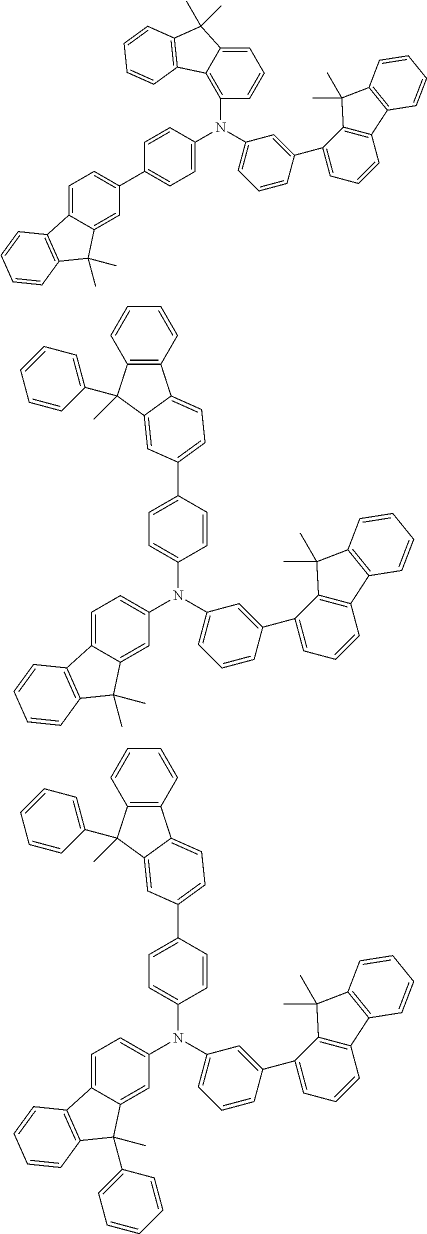

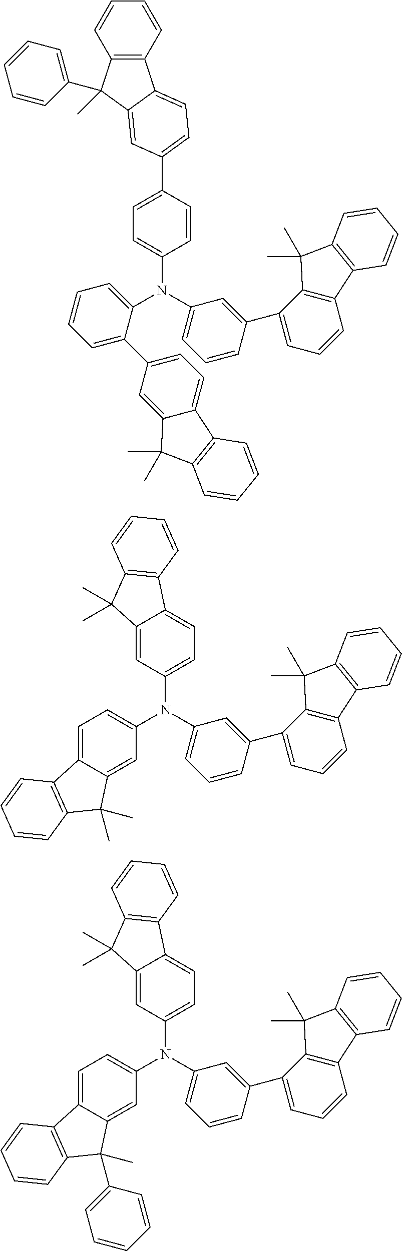

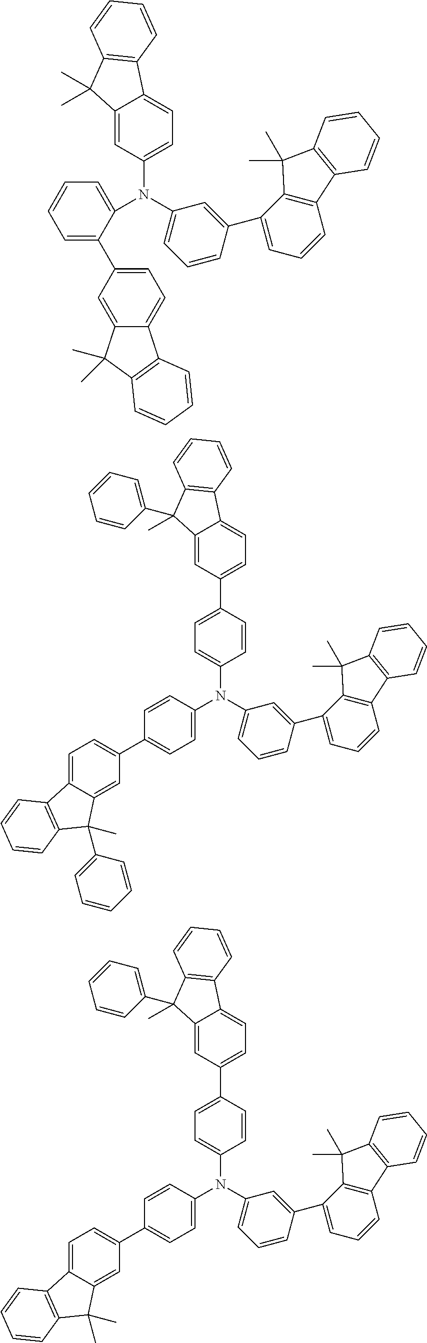

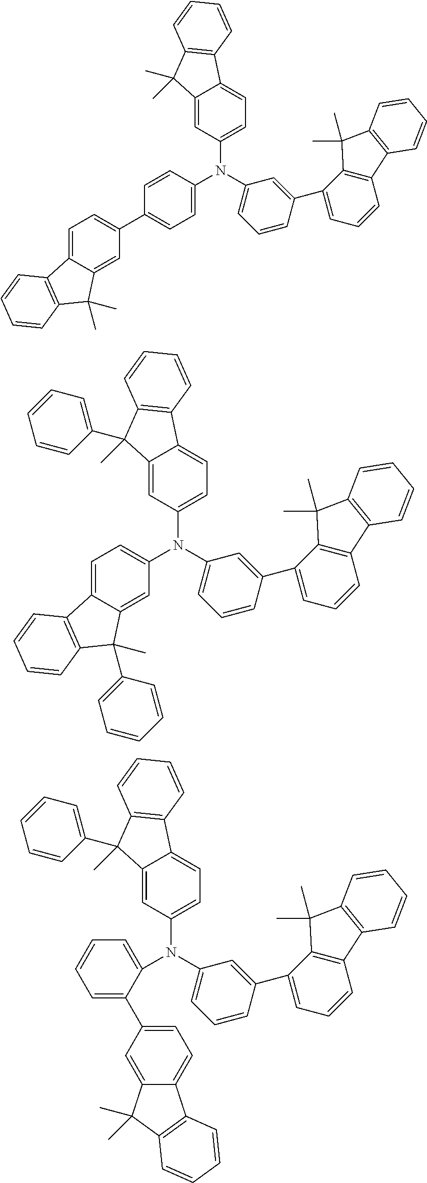

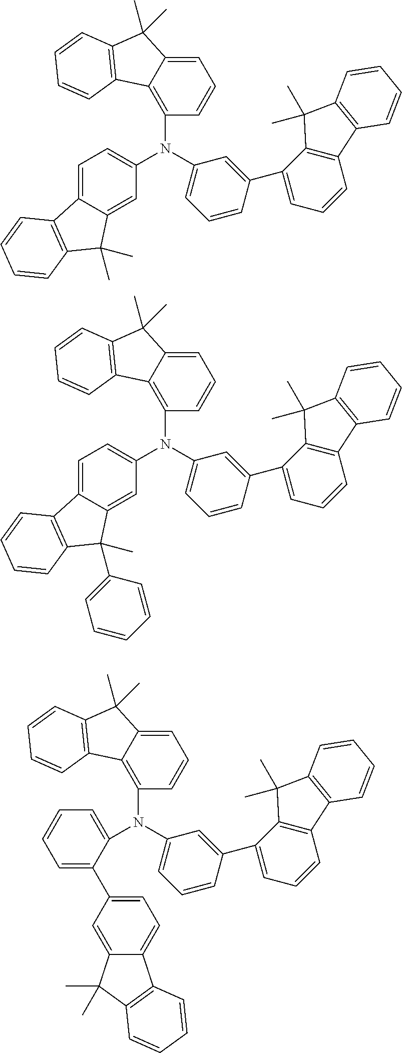

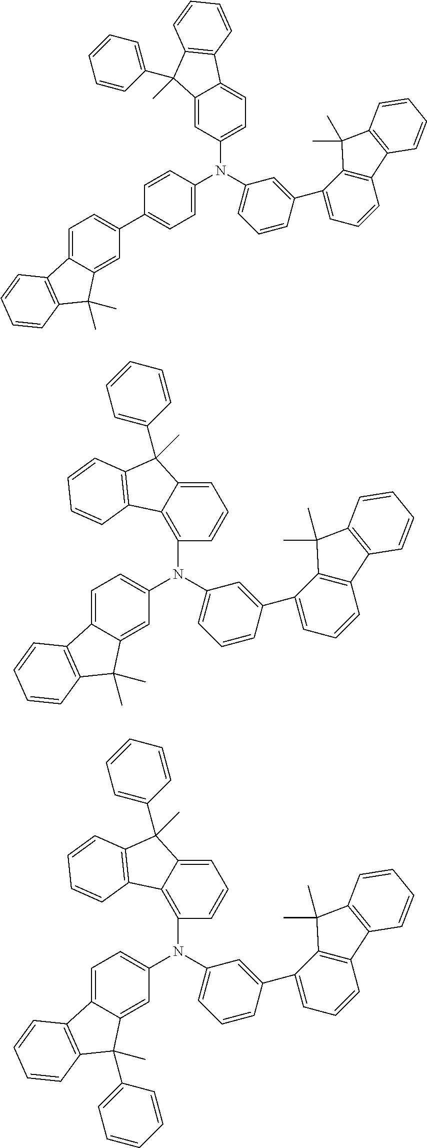

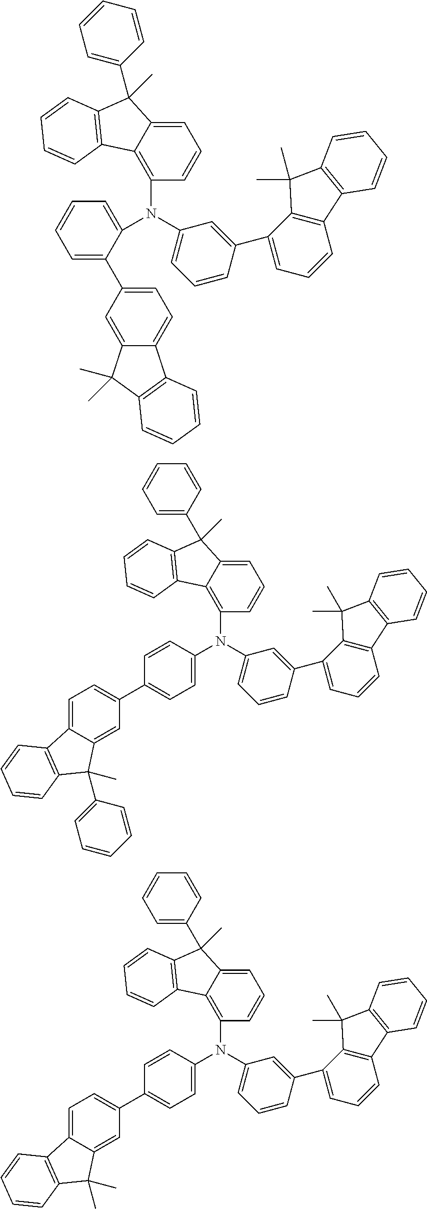

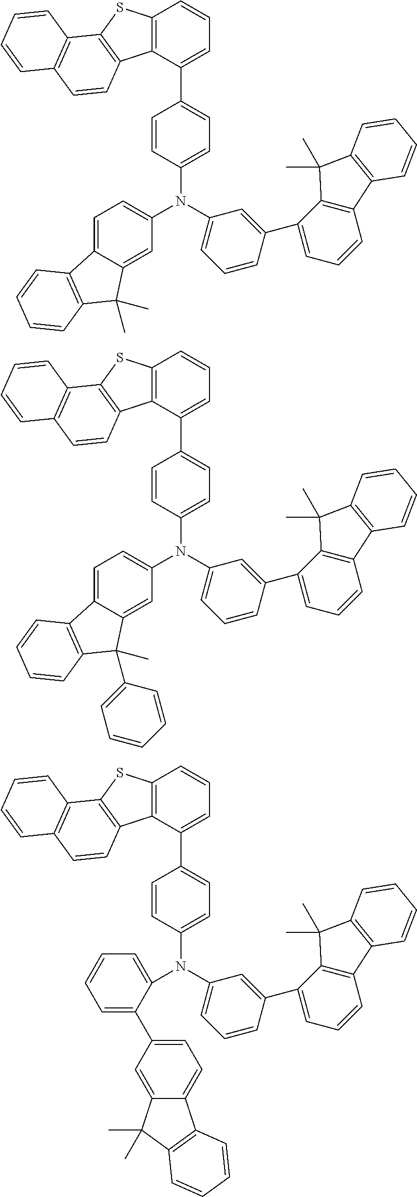

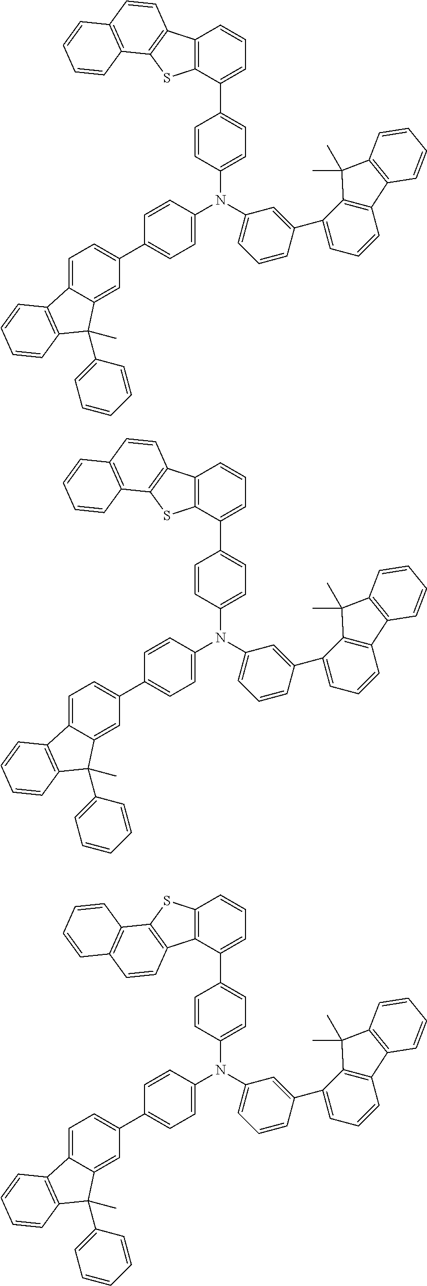

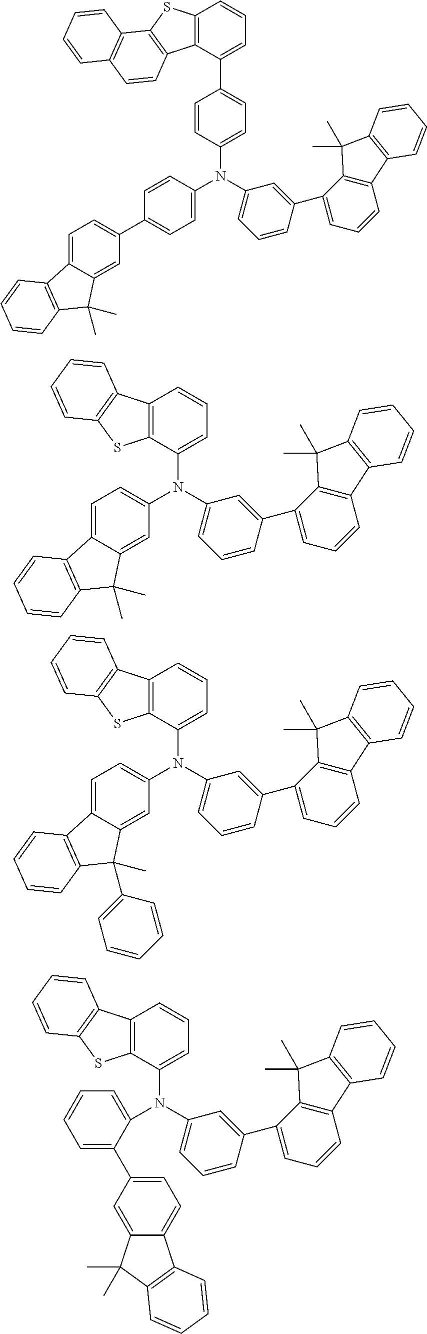

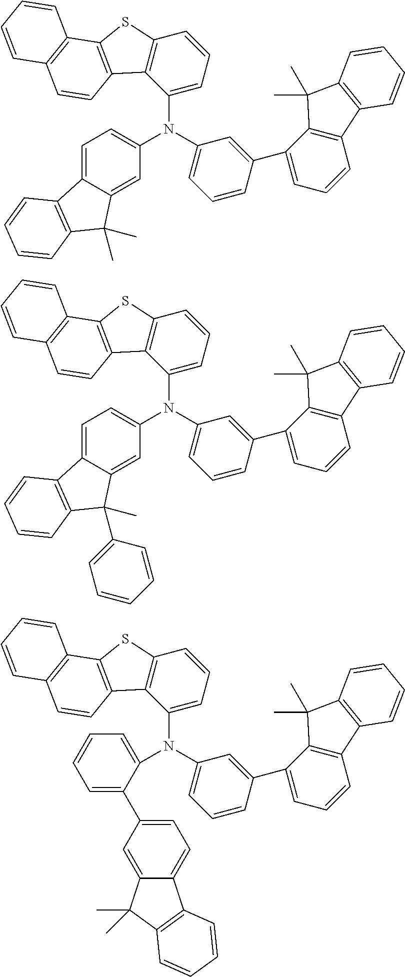

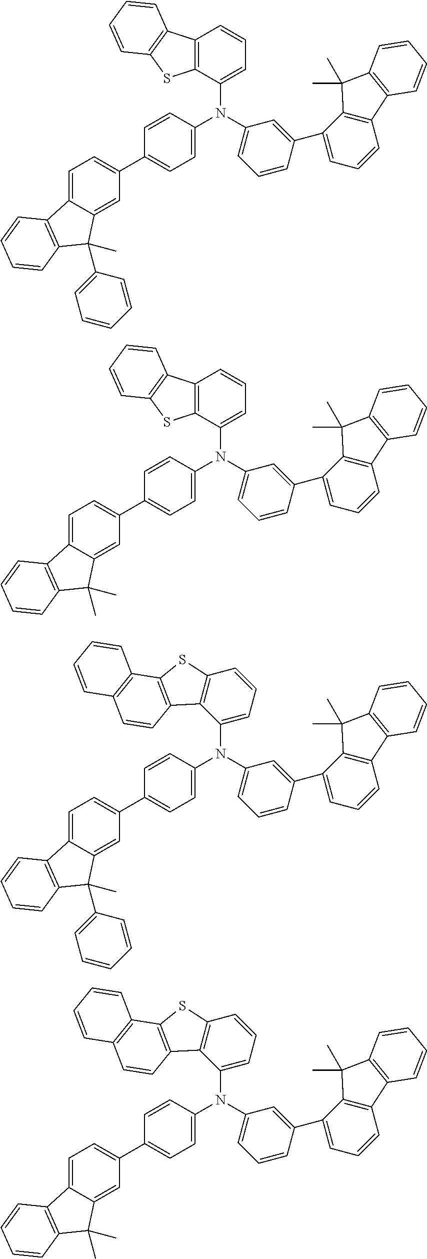

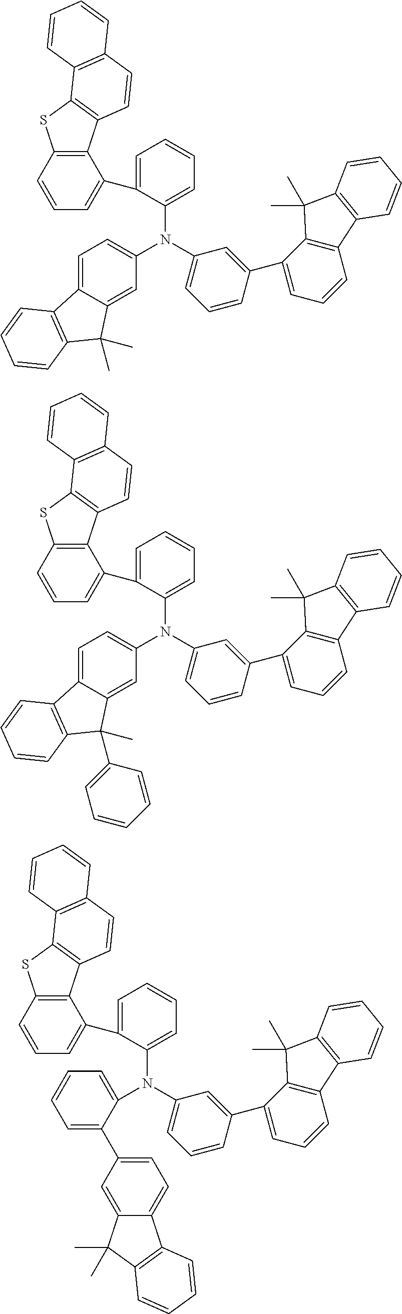

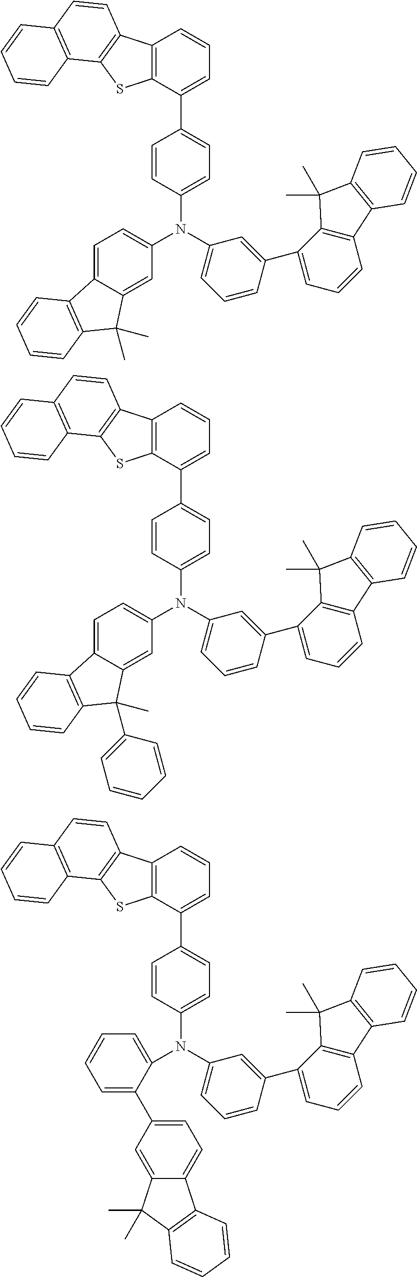

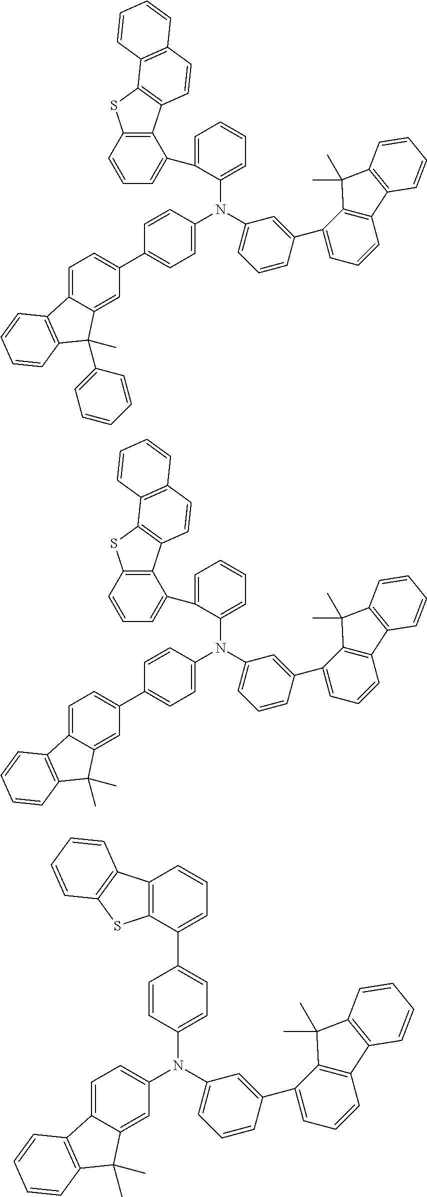

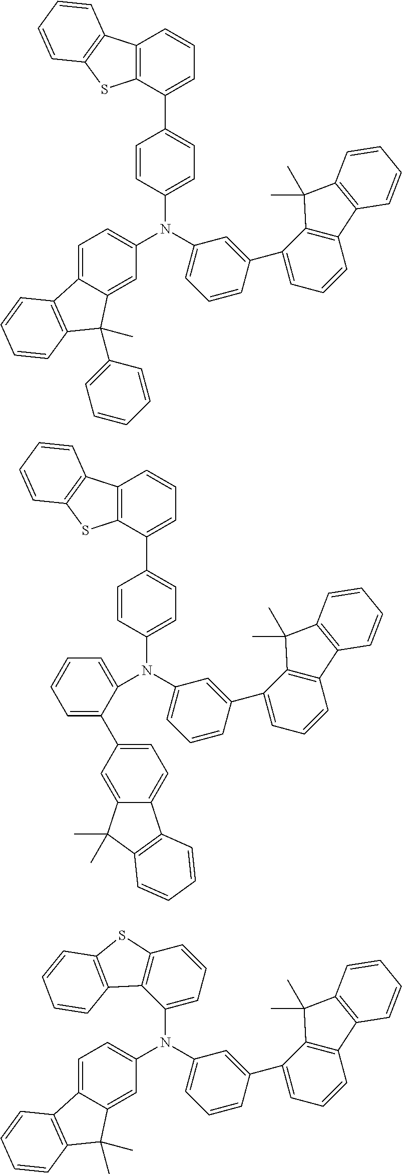

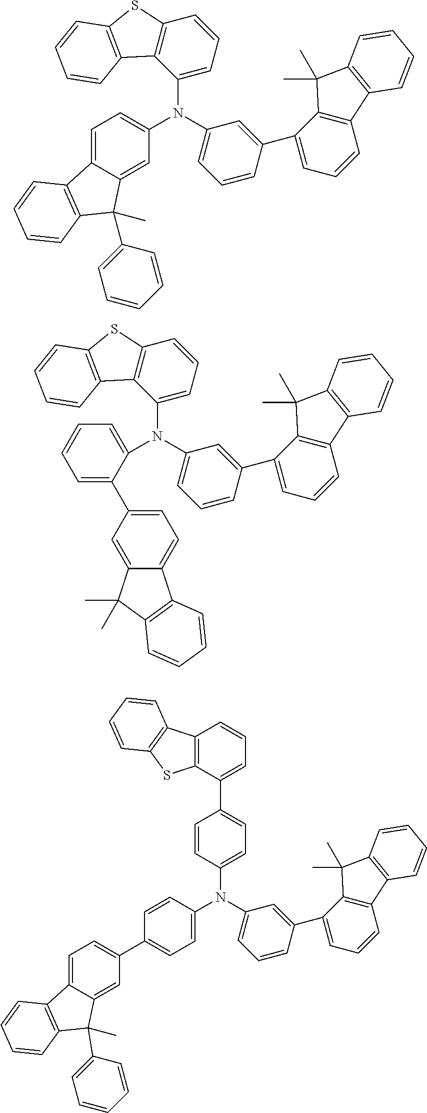

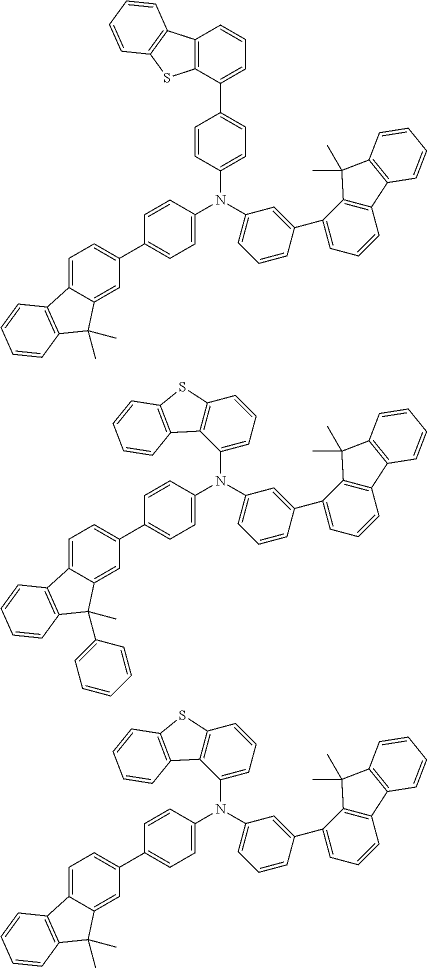

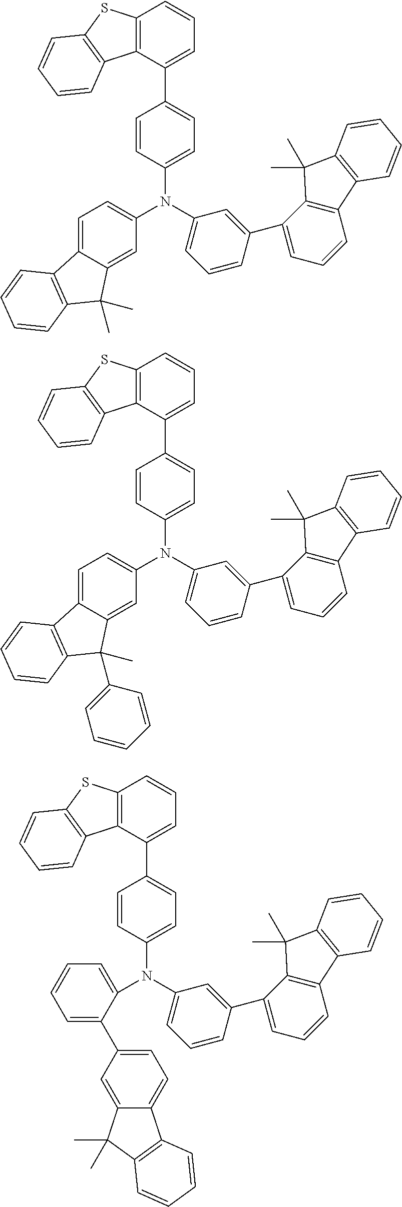

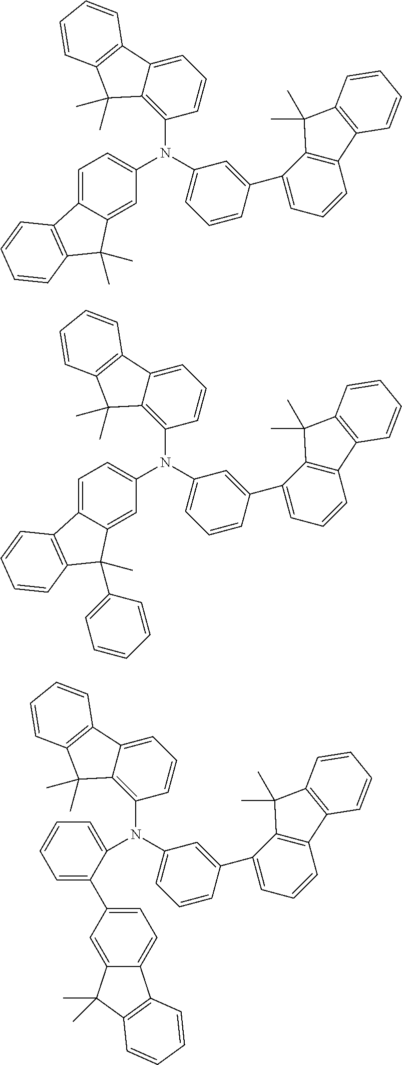

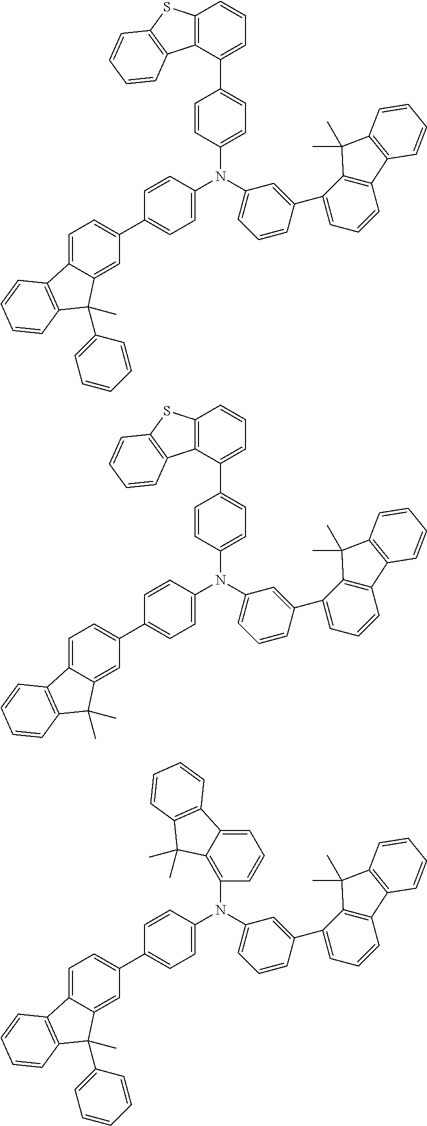 [SMILES: CC1(C)c2ccccc2-c2cccc(-c3cccc(N(c4ccc(-c5ccc6c(c5)C(C)(c5ccccc5)c5ccccc5-6)cc4)c4ccc(-c5cccc6sc7ccccc7c56)cc4)c3)c21 ; CC1(C)c2ccccc2-c2ccc(-c3ccc(N(c4ccc(-c5cccc6sc7ccccc7c56)cc4)c4cccc(-c5cccc6c5C(C)(C)c5ccccc5-6)c4)cc3)cc21 ; CC1(C)c2ccccc2-c2cccc(-c3cccc(N(c4ccc(-c5ccc6c(c5)C(C)(c5ccccc5)c5ccccc5-6)cc4)c4cccc5c4C(C)(C)c4ccccc4-5)c3)c21]

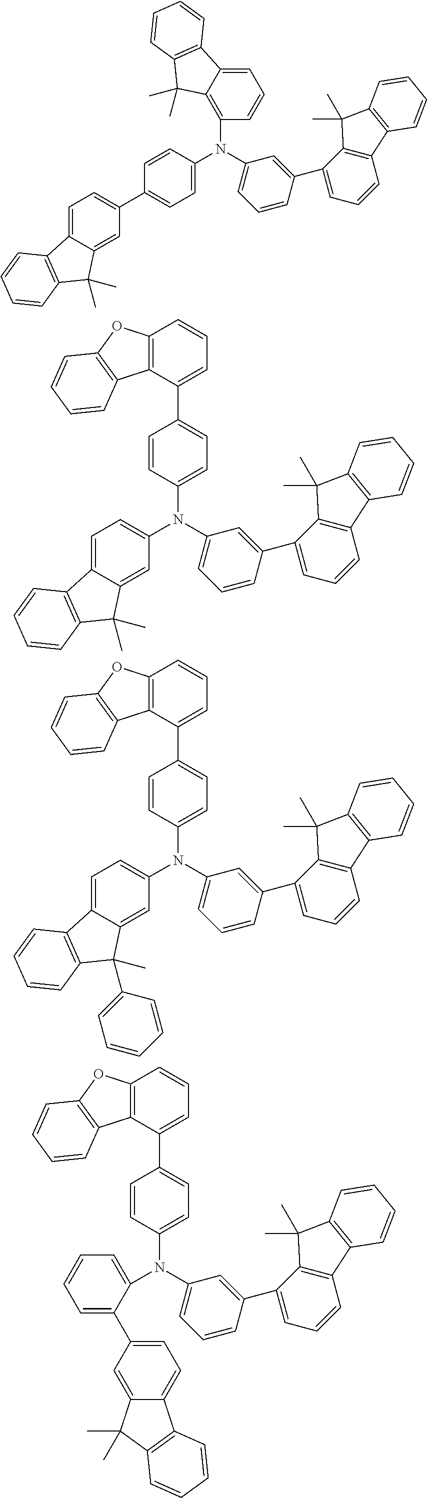

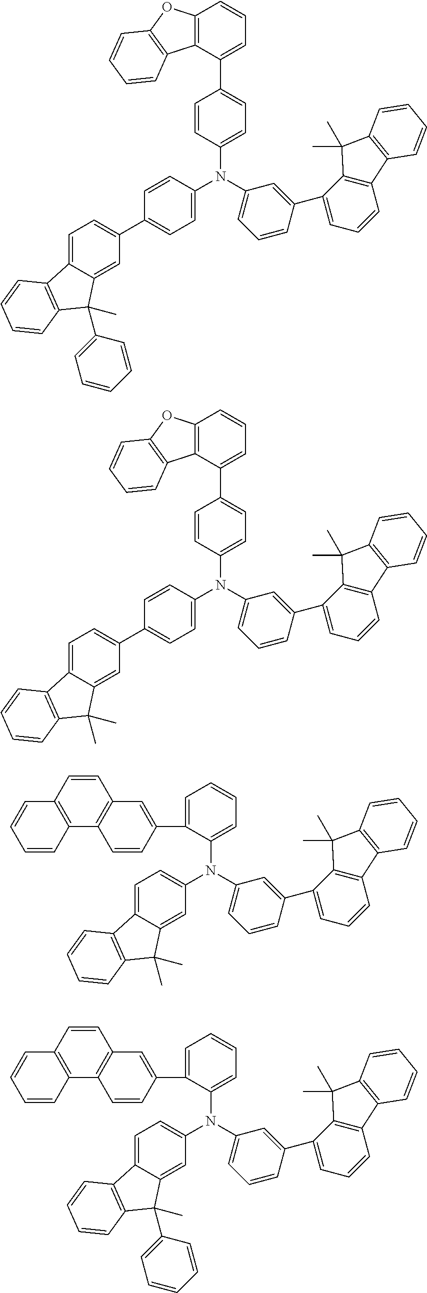

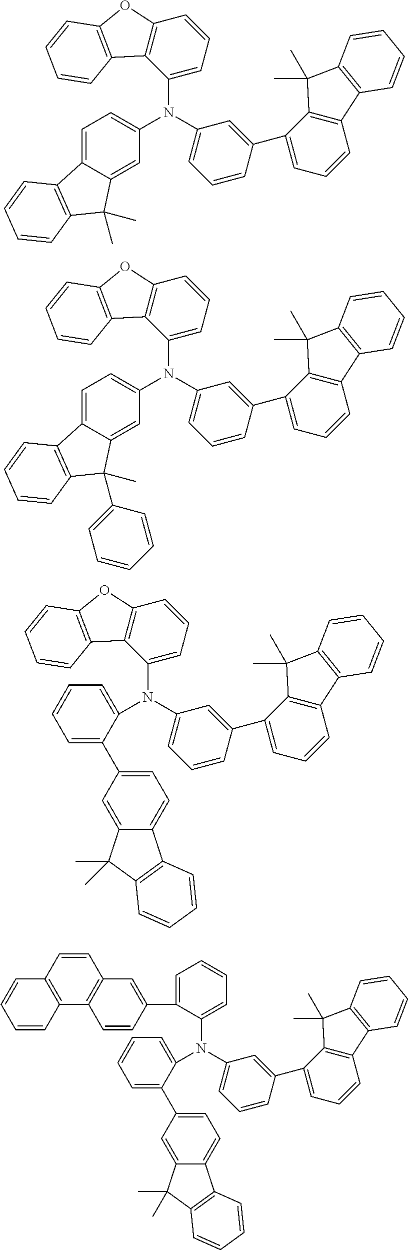

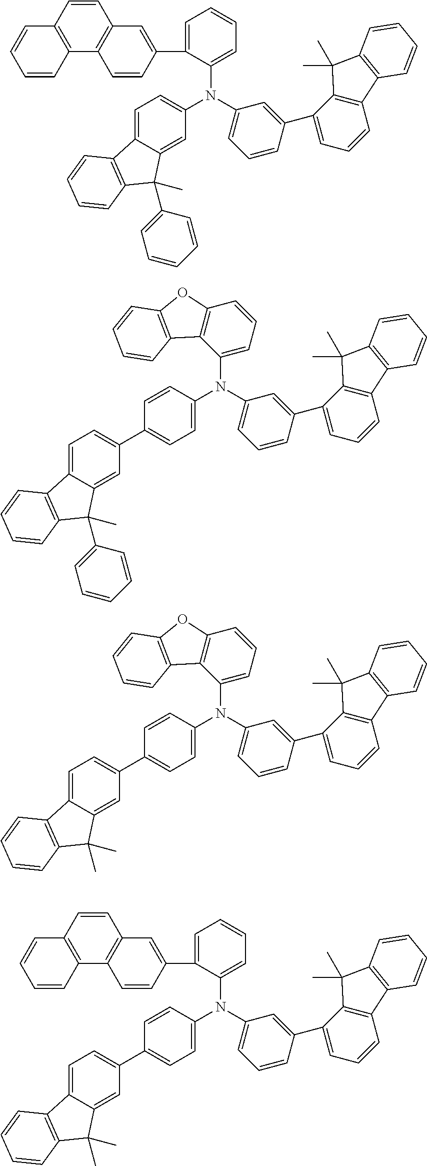

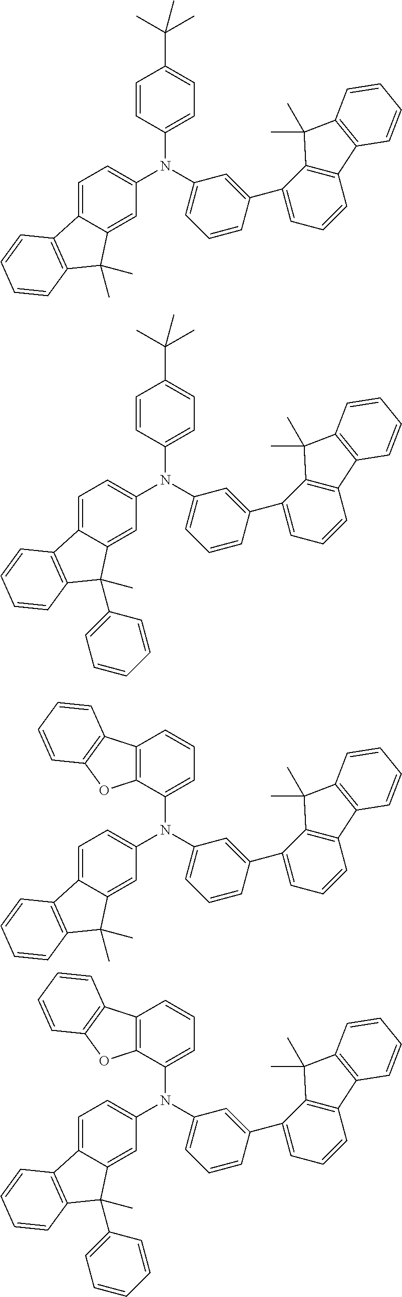

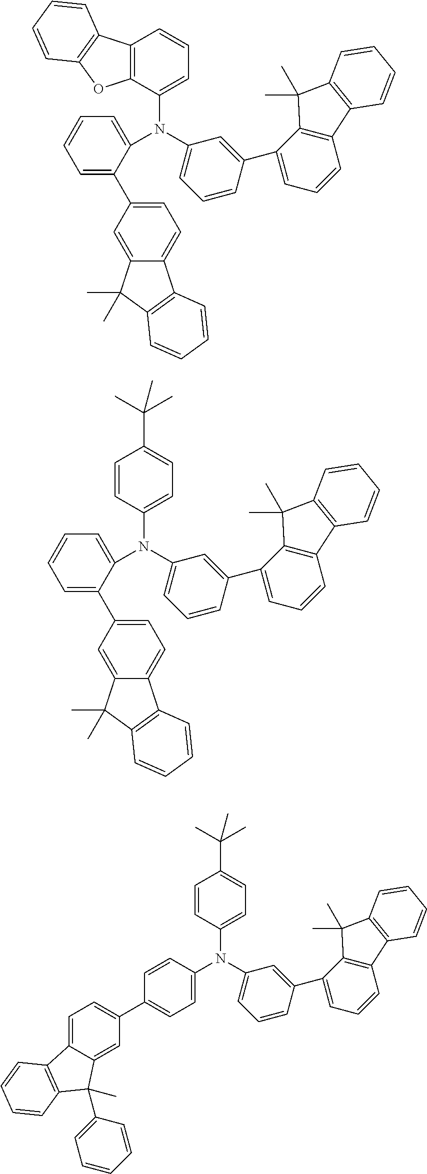

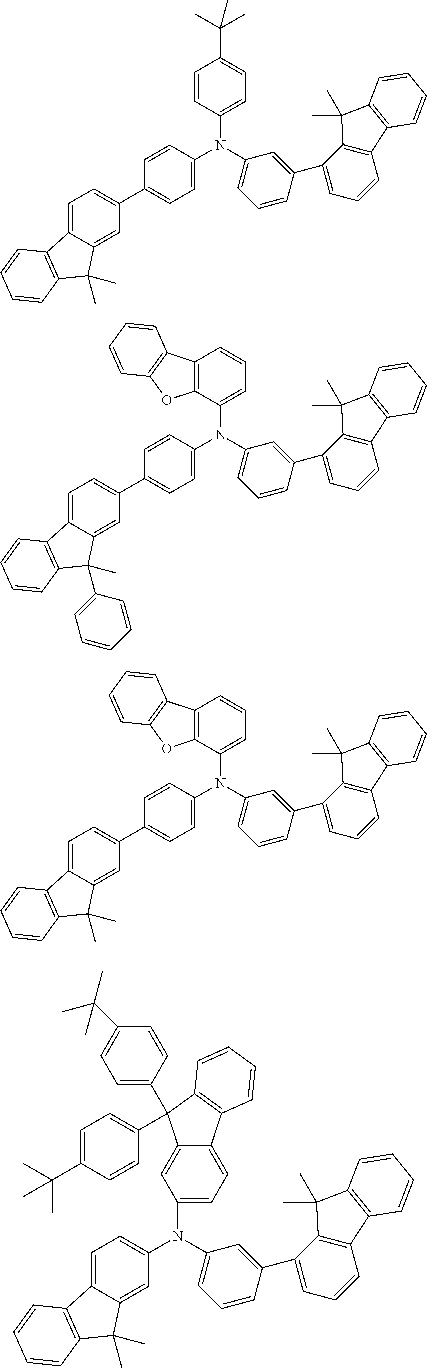

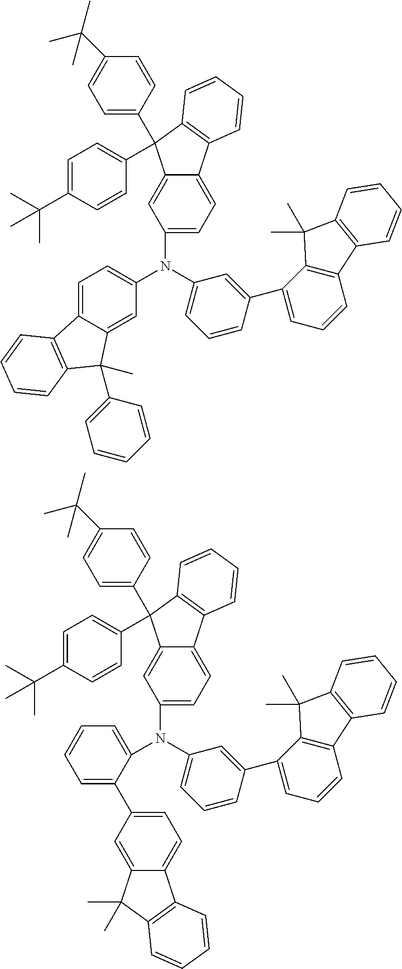

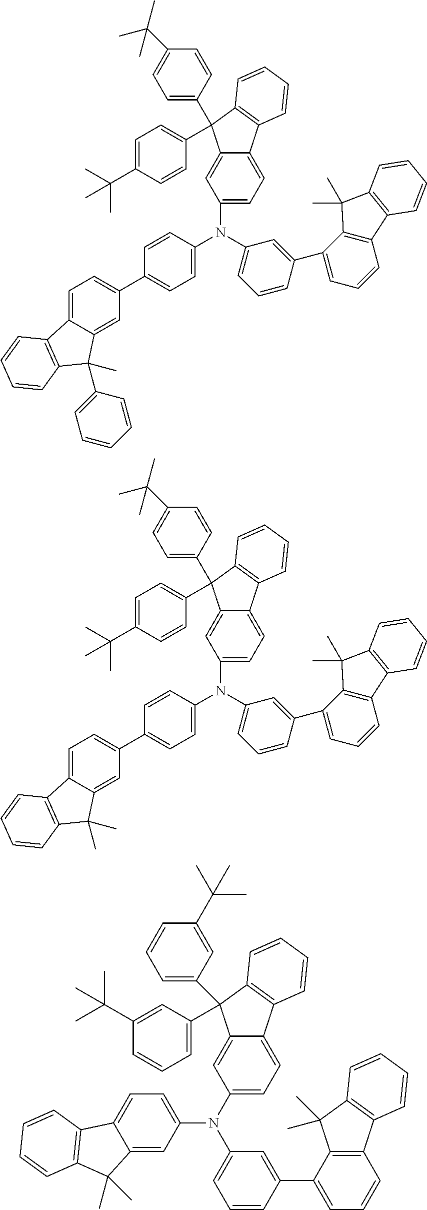

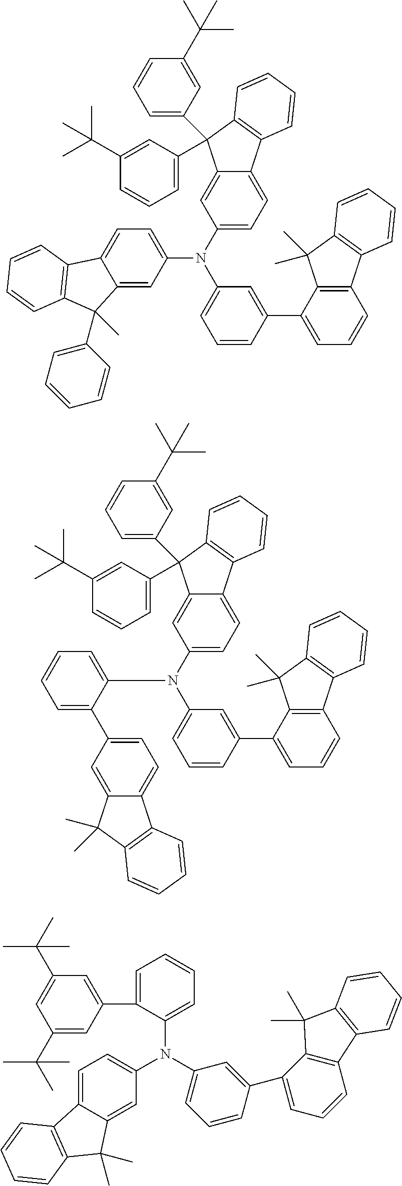 [SMILES: CC(C)(C)c1cccc(C2(c3cccc(C(C)(C)C)c3)c3ccccc3-c3ccc(N(c4cccc(-c5cccc6c5C(C)(C)c5ccccc5-6)c4)c4ccc5c(c4)C(C)(c4ccccc4)c4ccccc4-5)cc32)c1 ; CC(C)(C)c1cccc(C2(c3cccc(C(C)(C)C)c3)c3ccccc3-c3ccc(N(c4cccc(-c5cccc6c5C(C)(C)c5ccccc5-6)c4)c4ccccc4-c4ccc5c(c4)C(C)(C)c4ccccc4-5)cc32)c1 ; CC(C)(C)c1cc(-c2ccccc2N(c2cccc(-c3cccc4c3C(C)(C)c3ccccc3-4)c2)c2ccc3c(c2)C(C)(C)c2ccccc2-3)cc(C(C)(C)C)c1]

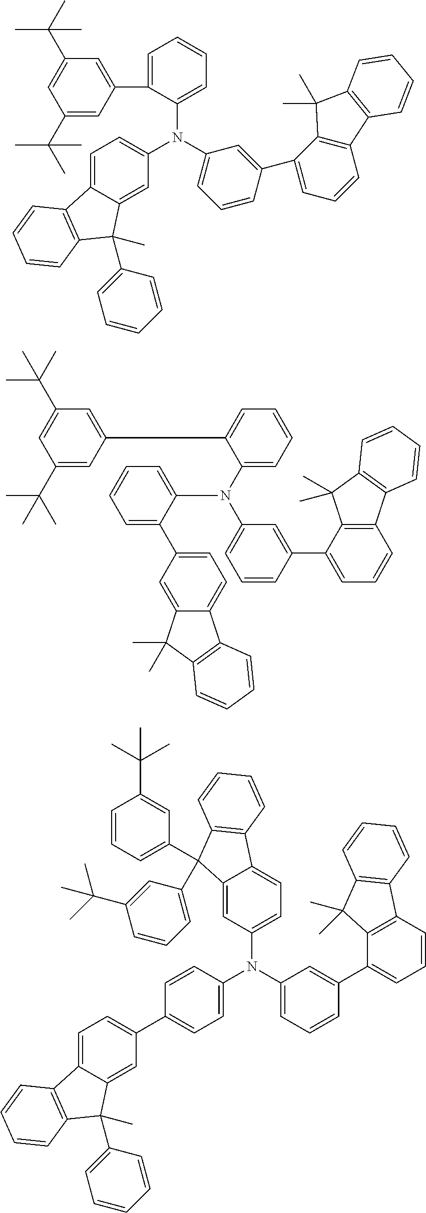 [SMILES: CC(C)(C)c1cc(-c2ccccc2N(c2cccc(-c3cccc4c3C(C)(C)c3ccccc3-4)c2)c2ccc3c(c2)C(C)(c2ccccc2)c2ccccc2-3)cc(C(C)(C)C)c1 ; CC(C)(C)c1cc(-c2ccccc2N(c2cccc(-c3cccc4c3C(C)(C)c3ccccc3-4)c2)c2ccccc2-c2ccc3c(c2)C(C)(C)c2ccccc2-3)cc(C(C)(C)C)c1 ; CC(C)(C)c1cccc(C2(c3cccc(C(C)(C)C)c3)c3ccccc3-c3ccc(N(c4ccc(-c5ccc6c(c5)C(C)(c5ccccc5)c5ccccc5-6)cc4)c4cccc(-c5cccc6c5C(C)(C)c5ccccc5-6)c4)cc32)c1]

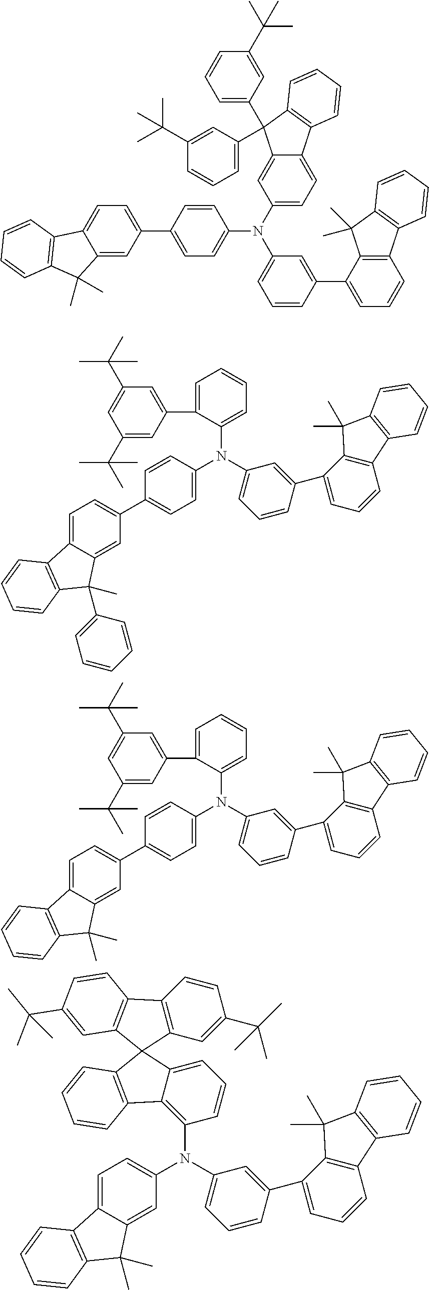

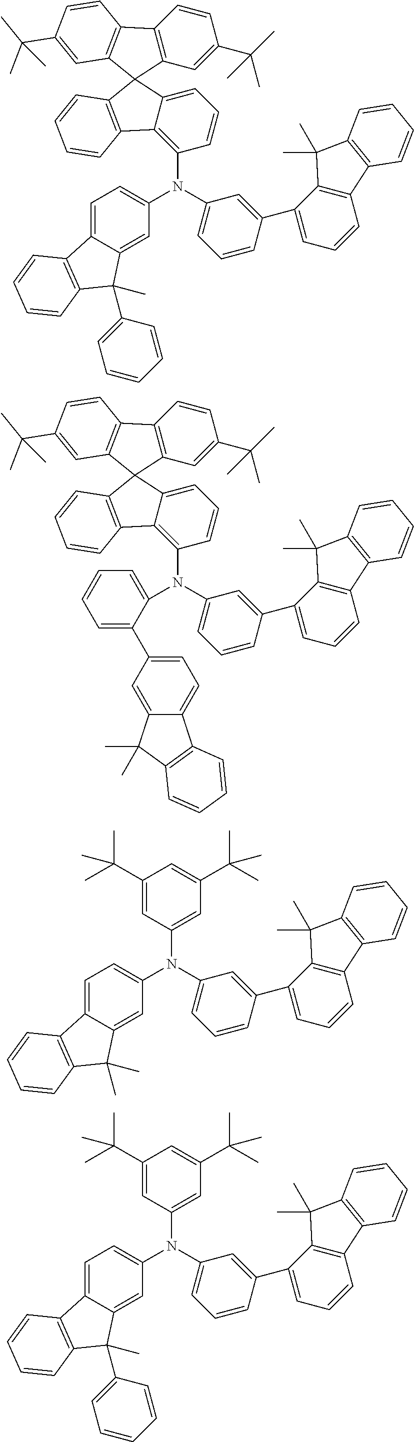

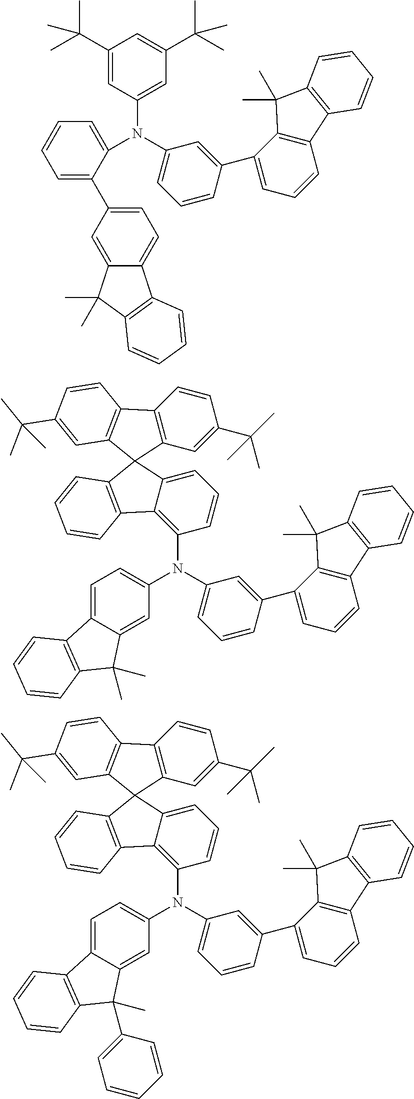

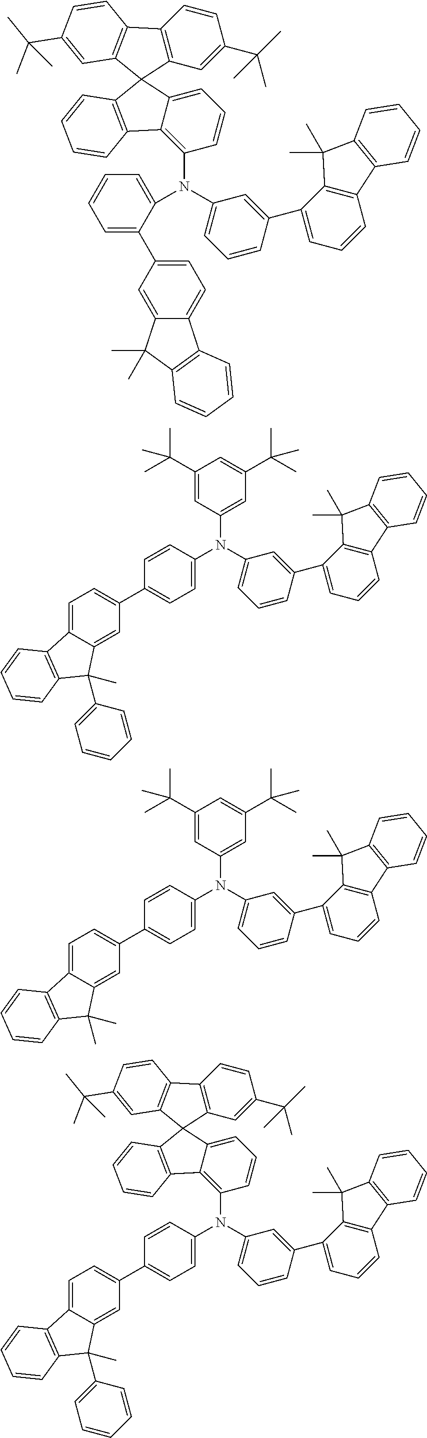

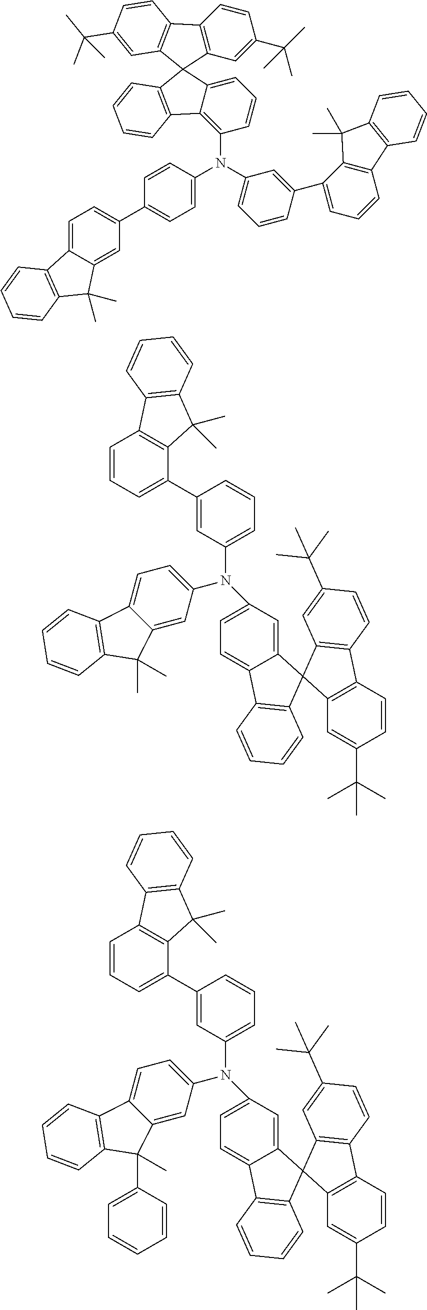

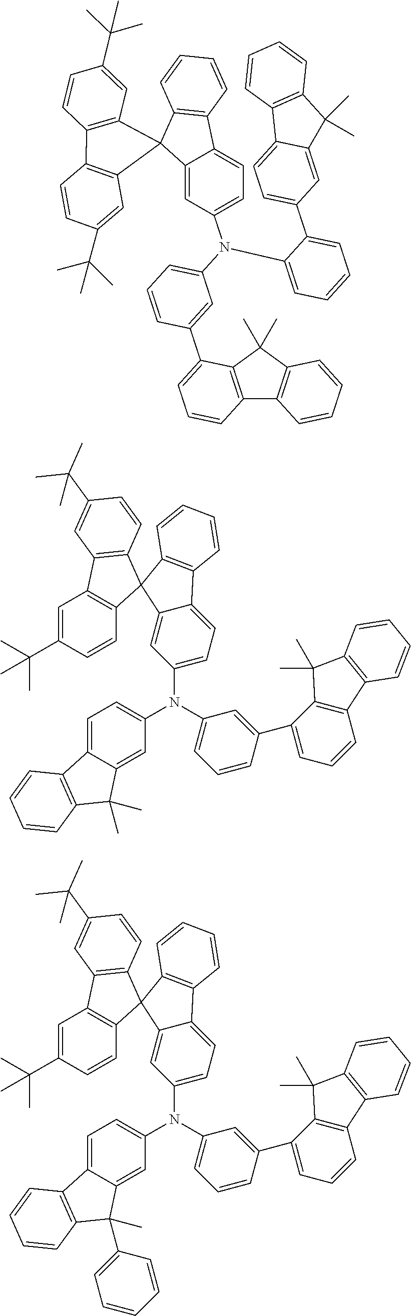

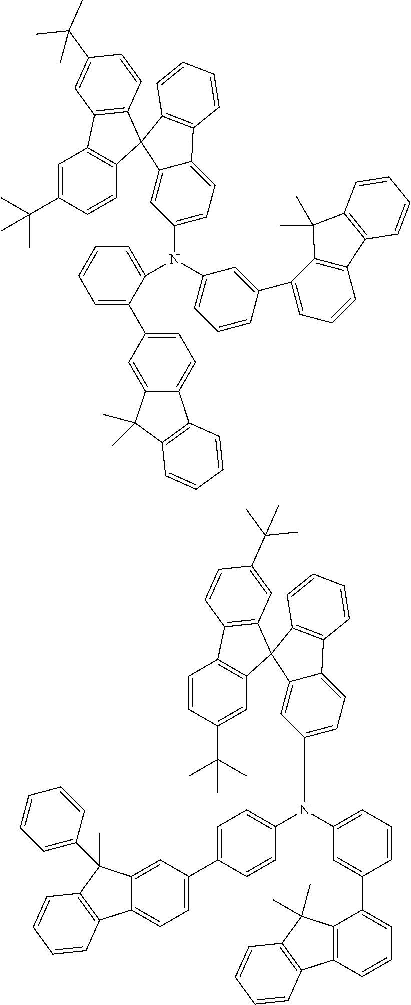

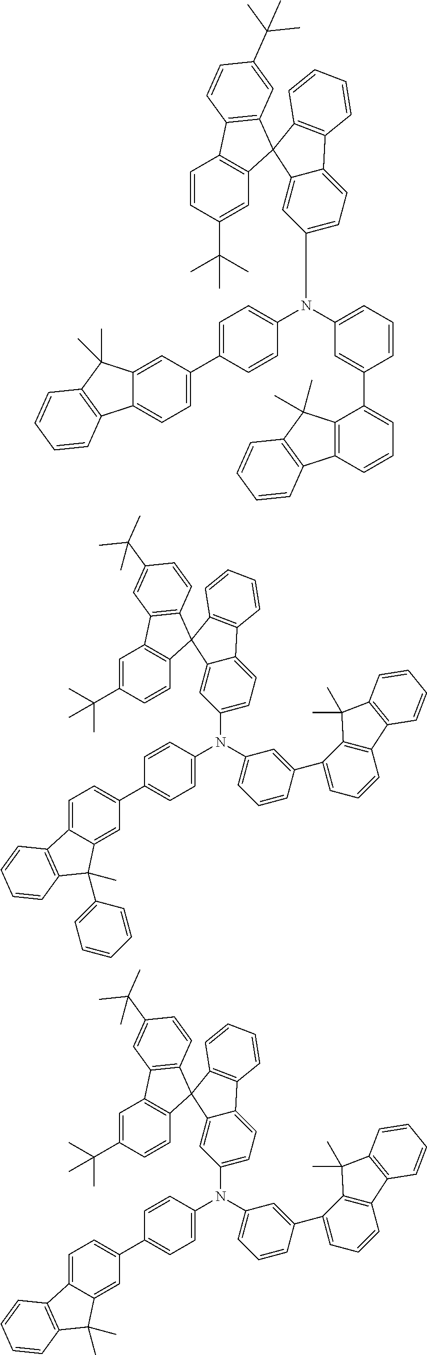

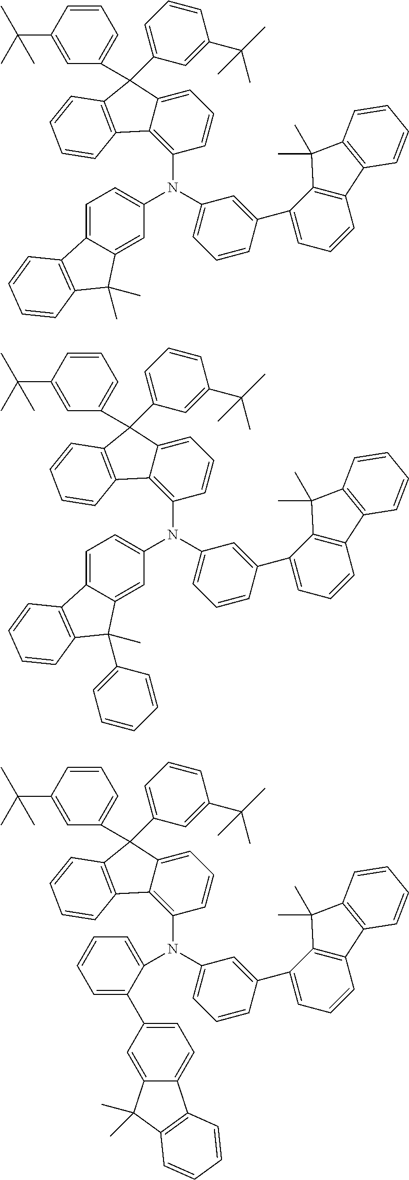

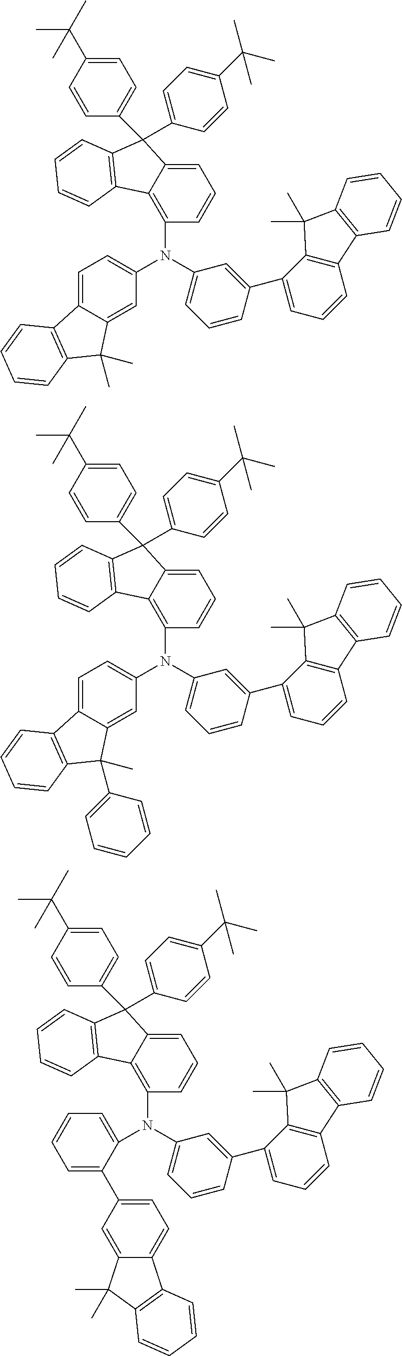

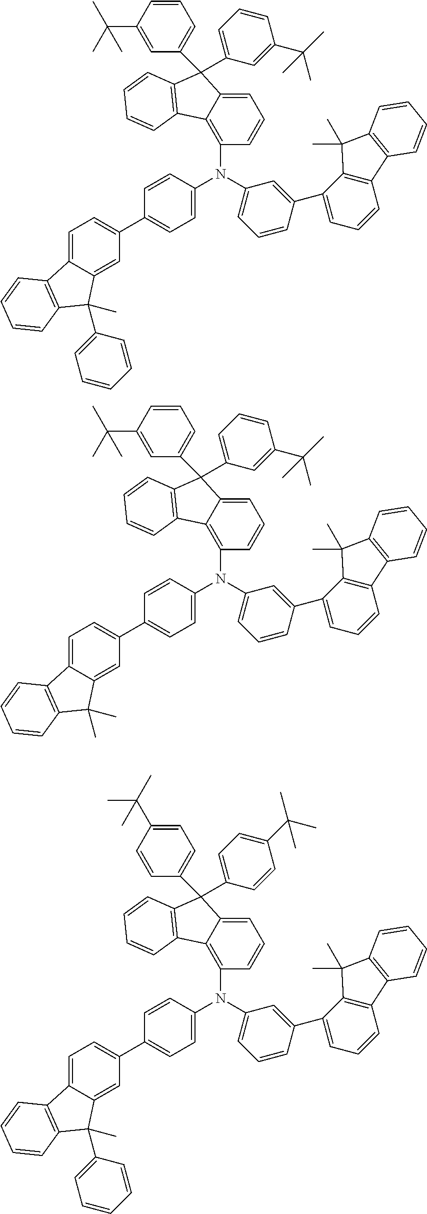

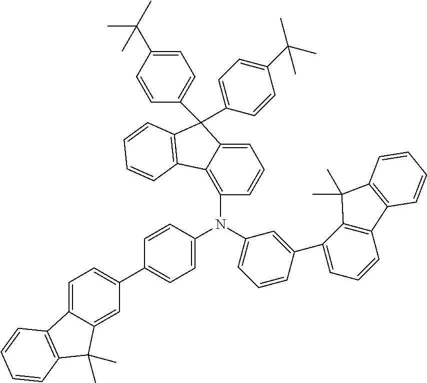 [SMILES: CC(C)(C)c1ccc(C2(c3ccc(C(C)(C)C)cc3)c3ccccc3-c3c(N(c4ccc(-c5ccc6c(c5)C(C)(C)c5ccccc5-6)cc4)c4cccc(-c5cccc6c5C(C)(C)c5ccccc5-6)c4)cccc32)cc1]

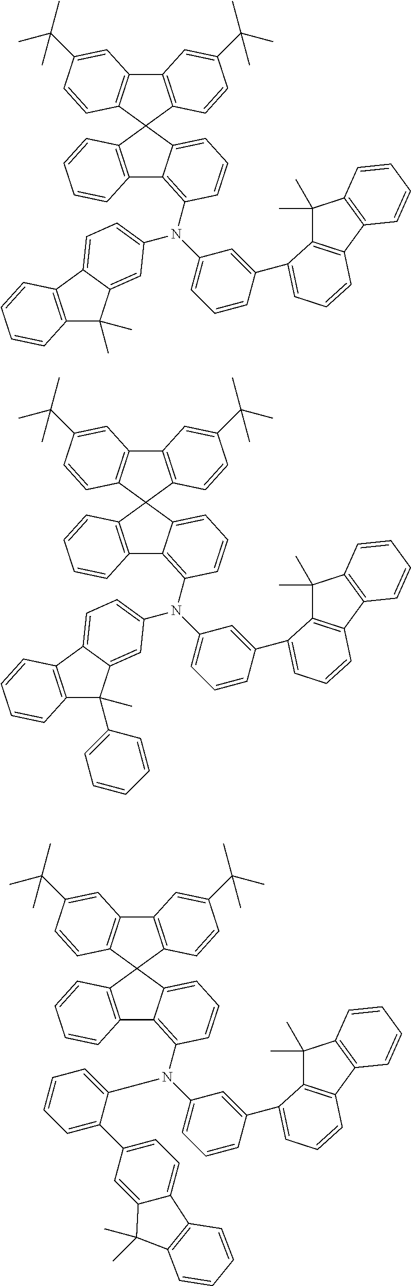

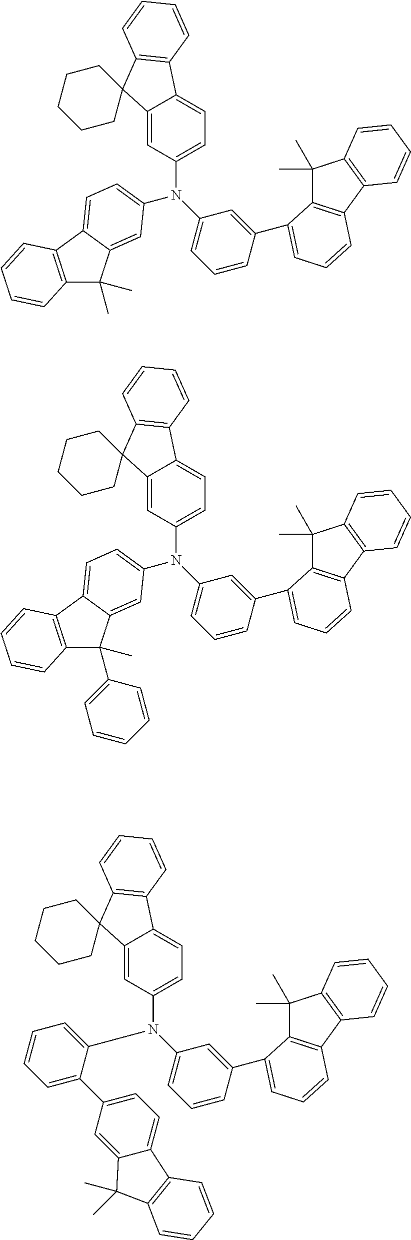 [SMILES: CC1(C)c2ccccc2-c2ccc(N(c3cccc(-c4cccc5c4C(C)(C)c4ccccc4-5)c3)c3ccc4c(c3)C3(CCCCC3)c3ccccc3-4)cc21 ; CC1(C)c2ccccc2-c2cccc(-c3cccc(N(c4ccc5c(c4)C4(CCCCC4)c4ccccc4-5)c4ccc5c(c4)C(C)(c4ccccc4)c4ccccc4-5)c3)c21 ; CC1(C)c2ccccc2-c2ccc(-c3ccccc3N(c3cccc(-c4cccc5c4C(C)(C)c4ccccc4-5)c3)c3ccc4c(c3)C3(CCCCC3)c3ccccc3-4)cc21]

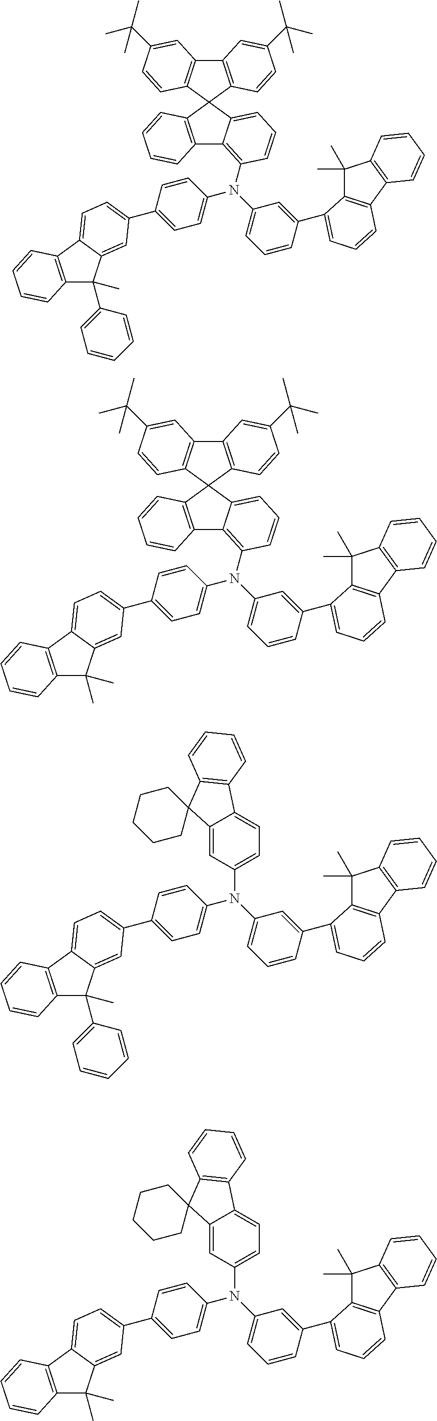

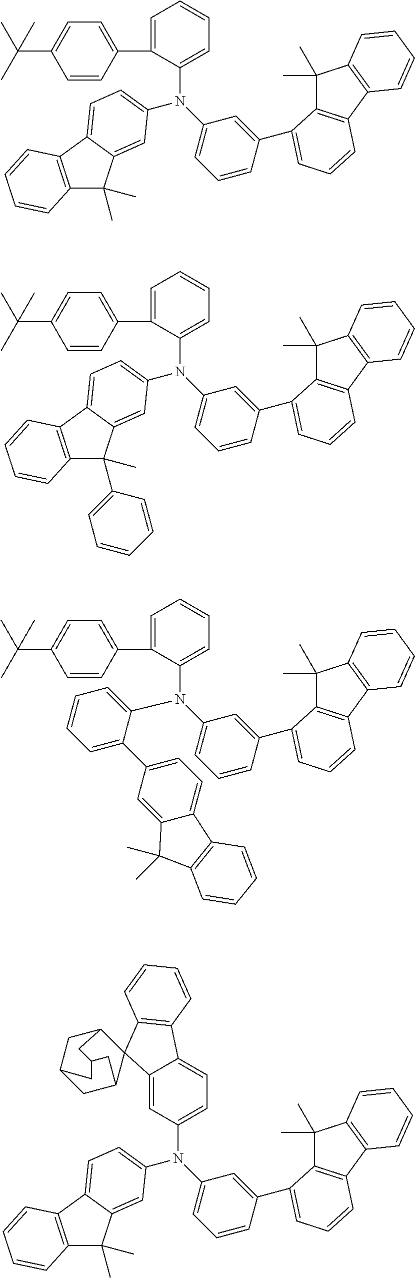 [SMILES: CC(C)(C)c1ccc(-c2ccccc2N(c2cccc(-c3cccc4c3C(C)(C)c3ccccc3-4)c2)c2ccc3c(c2)C(C)(C)c2ccccc2-3)cc1 ; CC(C)(C)c1ccc(-c2ccccc2N(c2cccc(-c3cccc4c3C(C)(C)c3ccccc3-4)c2)c2ccc3c(c2)C(C)(c2ccccc2)c2ccccc2-3)cc1 ; CC(C)(C)c1ccc(-c2ccccc2N(c2cccc(-c3cccc4c3C(C)(C)c3ccccc3-4)c2)c2ccccc2-c2ccc3c(c2)C(C)(C)c2ccccc2-3)cc1 ; CC1(C)c2ccccc2-c2ccc(N(c3cccc(-c4cccc5c4C(C)(C)c4ccccc4-5)c3)c3ccc4c(c3)C3(c5ccccc5-4)C4CC5CC(C4)CC3C5)cc21]

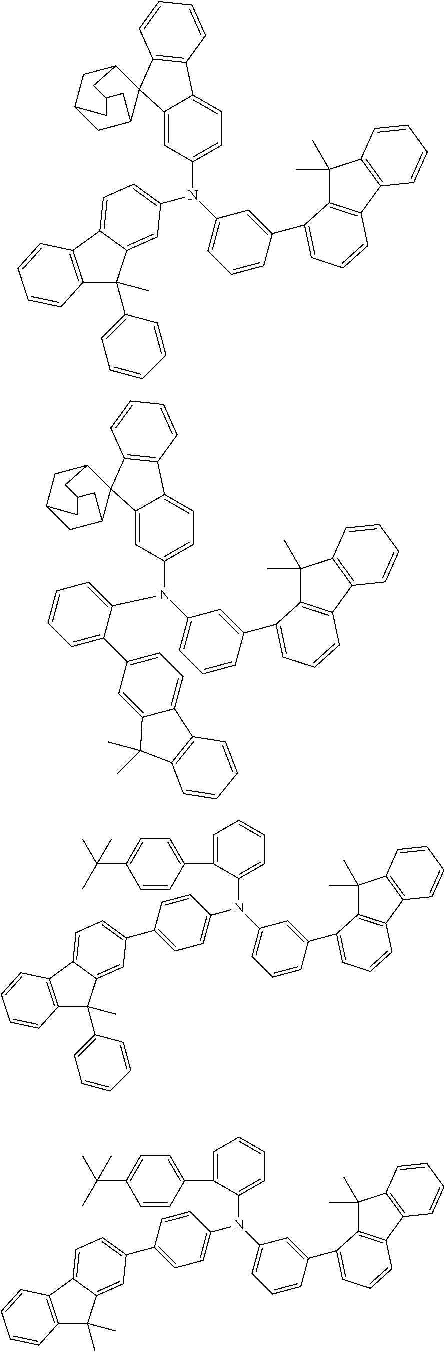

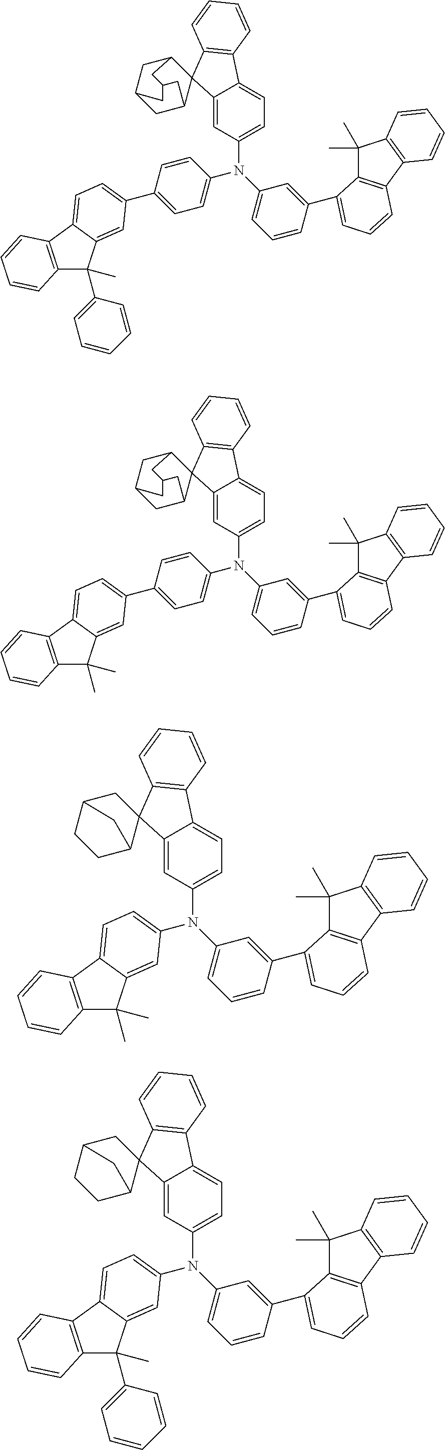 [SMILES: CC1(C)c2ccccc2-c2cccc(-c3cccc(N(c4ccc(-c5ccc6c(c5)C(C)(c5ccccc5)c5ccccc5-6)cc4)c4ccc5c(c4)C4(c6ccccc6-5)C5CC6CC(C5)CC4C6)c3)c21 ; CC1(C)c2ccccc2-c2ccc(-c3ccc(N(c4cccc(-c5cccc6c5C(C)(C)c5ccccc5-6)c4)c4ccc5c(c4)C4(c6ccccc6-5)C5CC6CC(C5)CC4C6)cc3)cc21 ; CC1(C)c2ccccc2-c2ccc(N(c3cccc(-c4cccc5c4C(C)(C)c4ccccc4-5)c3)c3ccc4c(c3)C3(CC5CCC3C5)c3ccccc3-4)cc21 ; CC1(C)c2ccccc2-c2cccc(-c3cccc(N(c4ccc5c(c4)C(C)(c4ccccc4)c4ccccc4-5)c4ccc5c(c4)C4(CC6CCC4C6)c4ccccc4-5)c3)c21]

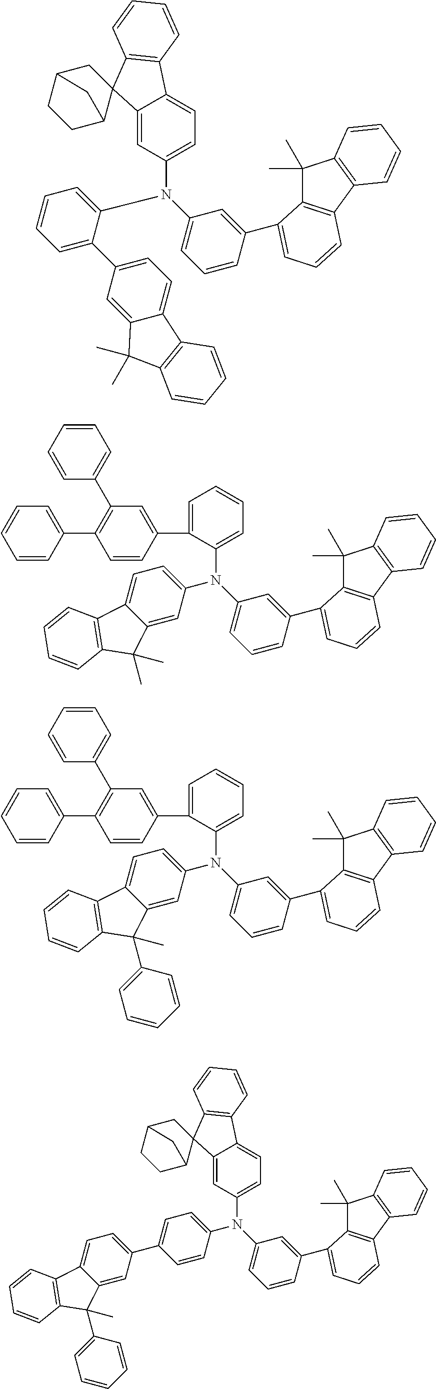

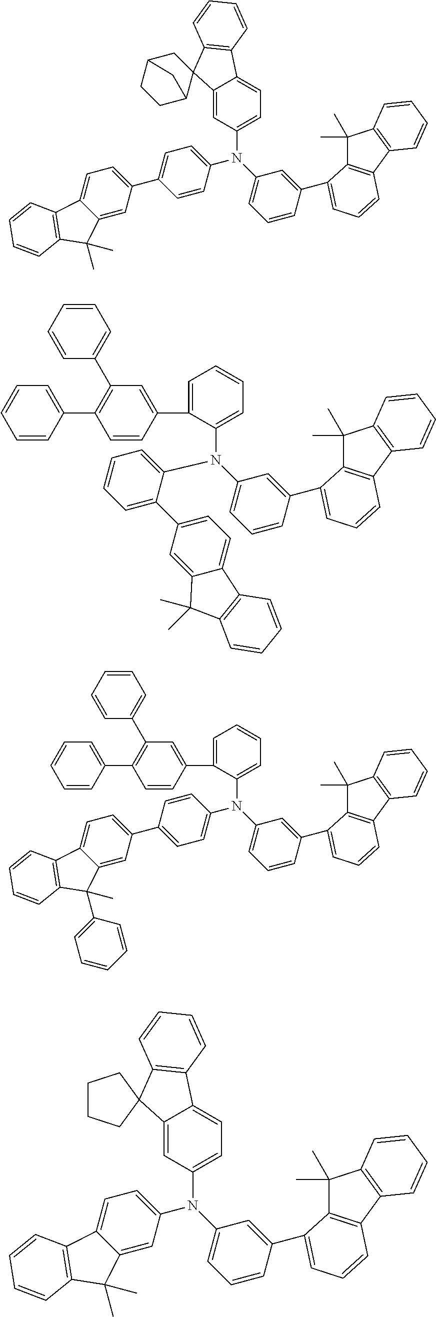

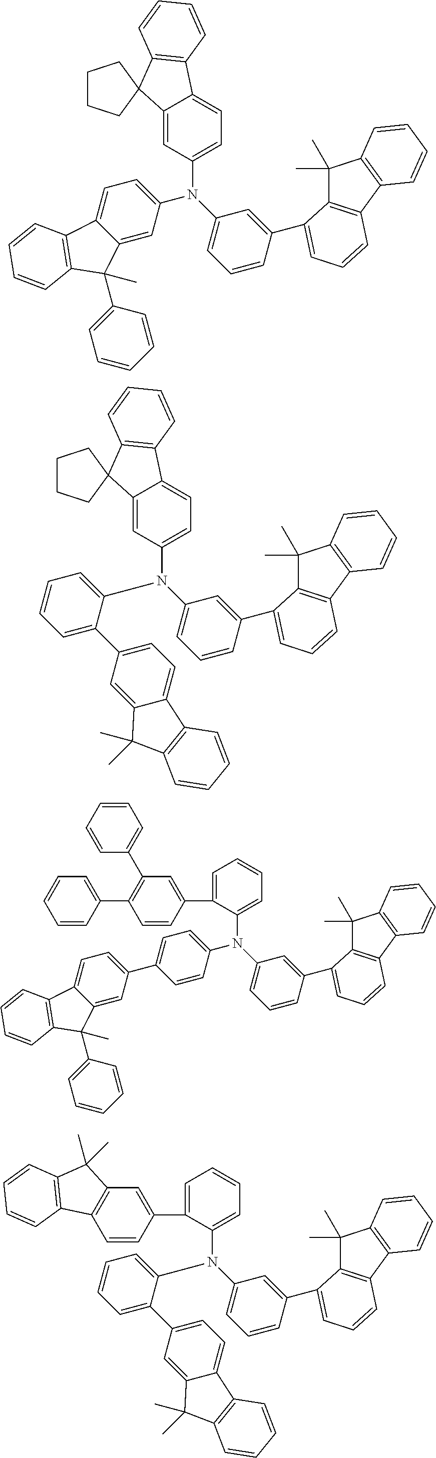

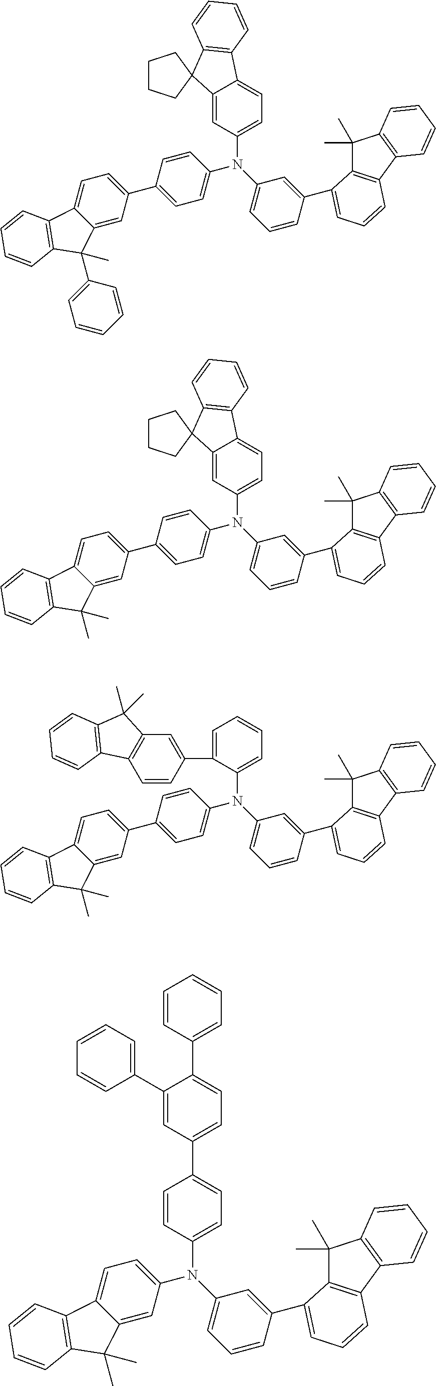 [SMILES: CC1(C)c2ccccc2-c2cccc(-c3cccc(N(c4ccc(-c5ccc6c(c5)C(C)(c5ccccc5)c5ccccc5-6)cc4)c4ccc5c(c4)C4(CCCC4)c4ccccc4-5)c3)c21 ; CC1(C)c2ccccc2-c2ccc(-c3ccc(N(c4cccc(-c5cccc6c5C(C)(C)c5ccccc5-6)c4)c4ccc5c(c4)C4(CCCC4)c4ccccc4-5)cc3)cc21 ; CC1(C)c2ccccc2-c2ccc(-c3ccc(N(c4cccc(-c5cccc6c5C(C)(C)c5ccccc5-6)c4)c4ccccc4-c4ccc5c(c4)C(C)(C)c4ccccc4-5)cc3)cc21 ; CC1(C)c2ccccc2-c2ccc(N(c3ccc(-c4ccc(-c5ccccc5)c(-c5ccccc5)c4)cc3)c3cccc(-c4cccc5c4C(C)(C)c4ccccc4-5)c3)cc21]

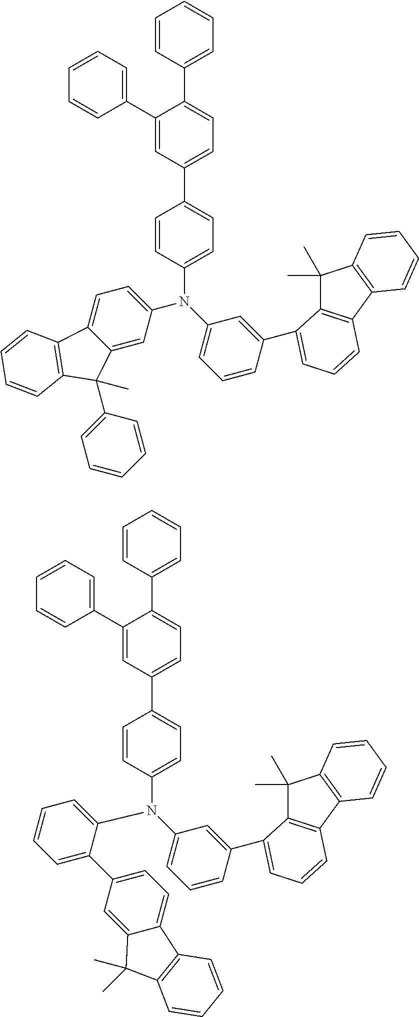

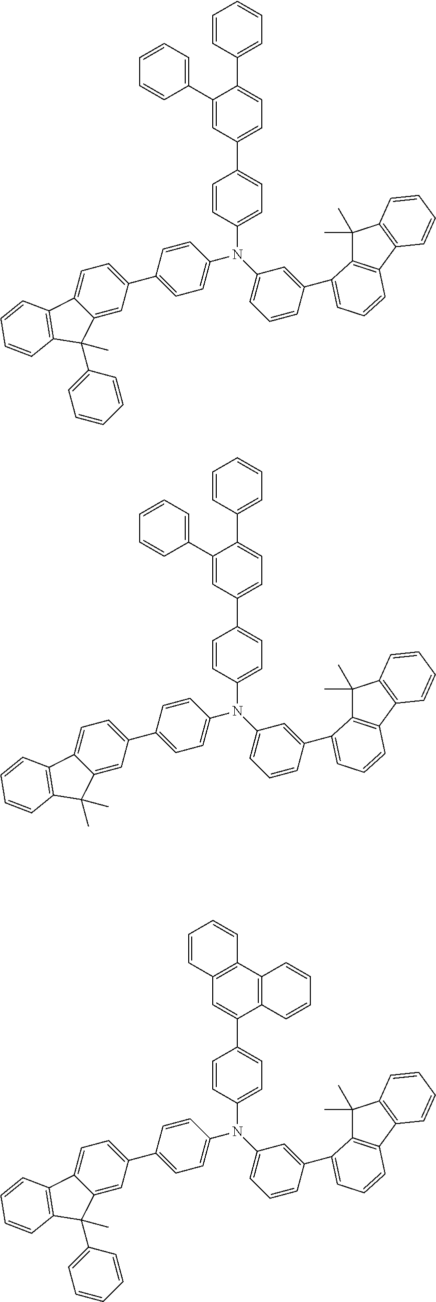 [SMILES: CC1(C)c2ccccc2-c2cccc(-c3cccc(N(c4ccc(-c5ccc(-c6ccccc6)c(-c6ccccc6)c5)cc4)c4ccc(-c5ccc6c(c5)C(C)(c5ccccc5)c5ccccc5-6)cc4)c3)c21 ; CC1(C)c2ccccc2-c2ccc(-c3ccc(N(c4ccc(-c5ccc(-c6ccccc6)c(-c6ccccc6)c5)cc4)c4cccc(-c5cccc6c5C(C)(C)c5ccccc5-6)c4)cc3)cc21 ; CC1(C)c2ccccc2-c2cccc(-c3cccc(N(c4ccc(-c5ccc6c(c5)C(C)(c5ccccc5)c5ccccc5-6)cc4)c4ccc(-c5cc6ccccc6c6ccccc56)cc4)c3)c21]

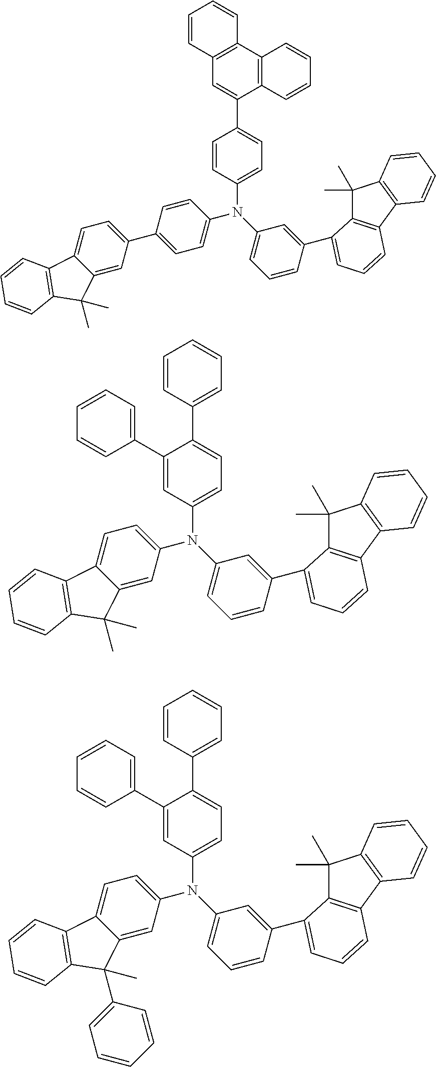 [SMILES: CC1(C)c2ccccc2-c2ccc(-c3ccc(N(c4ccc(-c5cc6ccccc6c6ccccc56)cc4)c4cccc(-c5cccc6c5C(C)(C)c5ccccc5-6)c4)cc3)cc21 ; CC1(C)c2ccccc2-c2ccc(N(c3cccc(-c4cccc5c4C(C)(C)c4ccccc4-5)c3)c3ccc(-c4ccccc4)c(-c4ccccc4)c3)cc21 ; CC1(C)c2ccccc2-c2cccc(-c3cccc(N(c4ccc(-c5ccccc5)c(-c5ccccc5)c4)c4ccc5c(c4)C(C)(c4ccccc4)c4ccccc4-5)c3)c21]

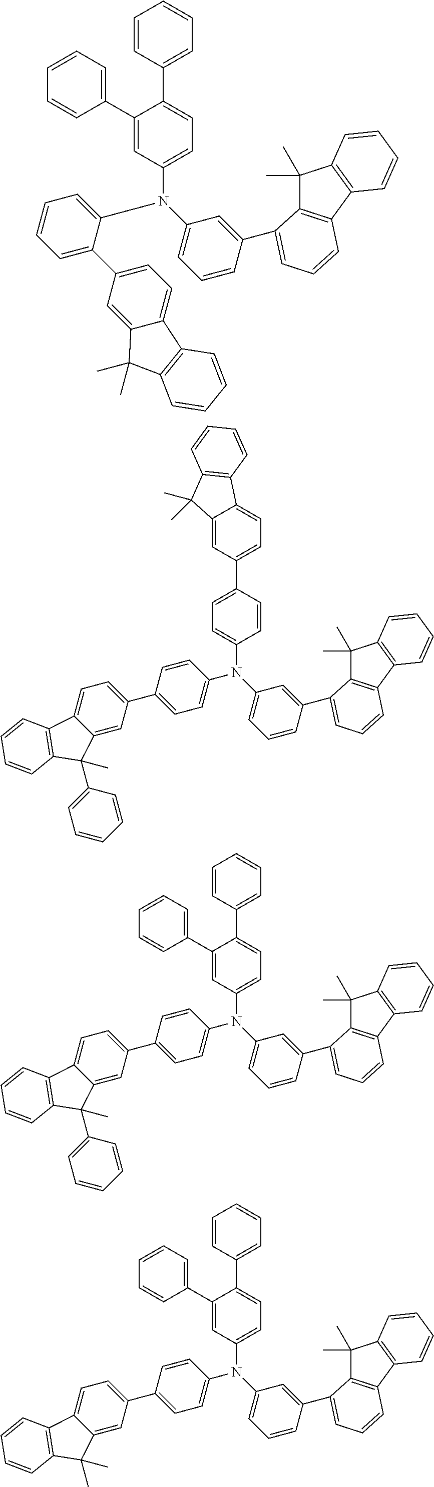

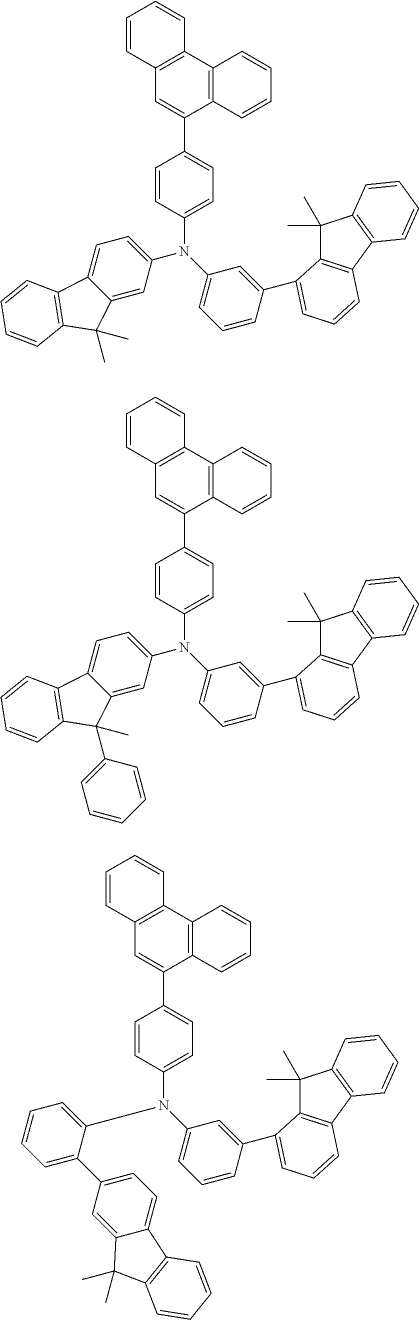

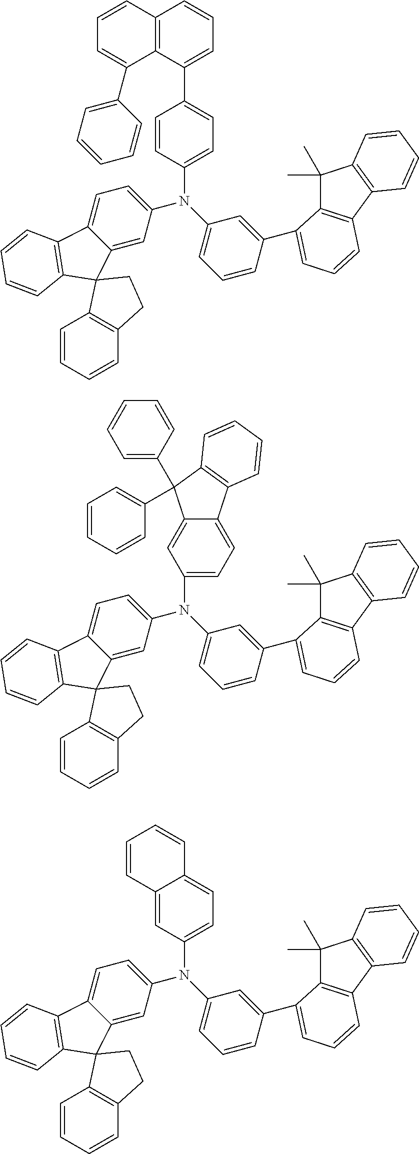

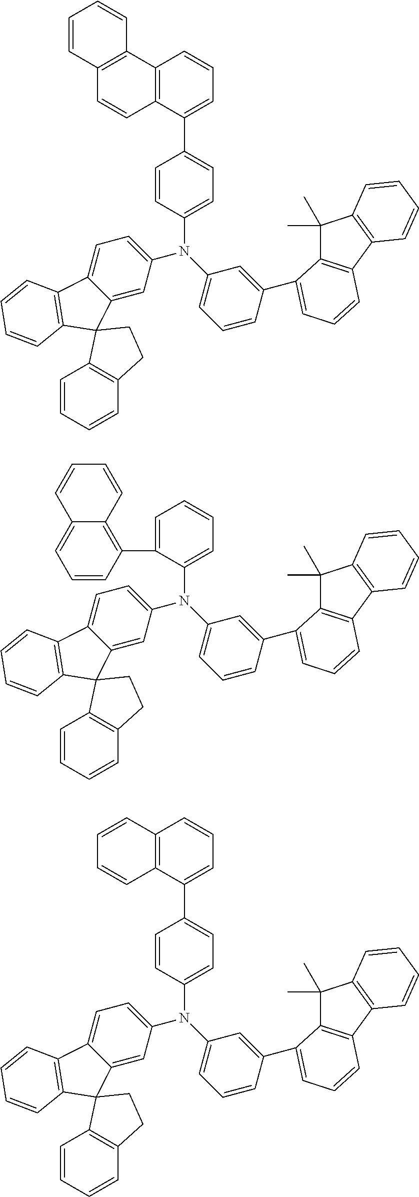

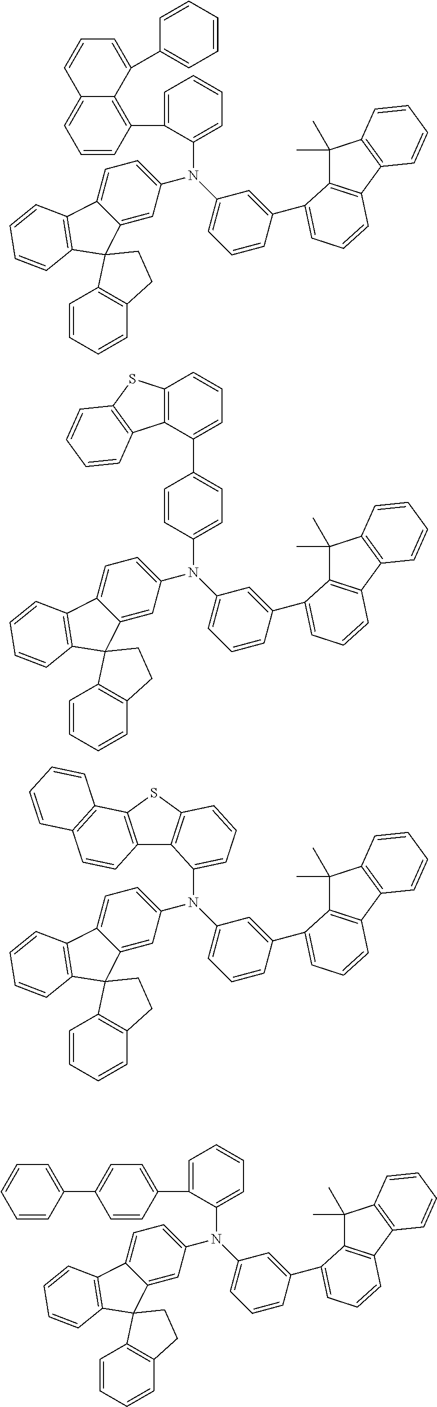

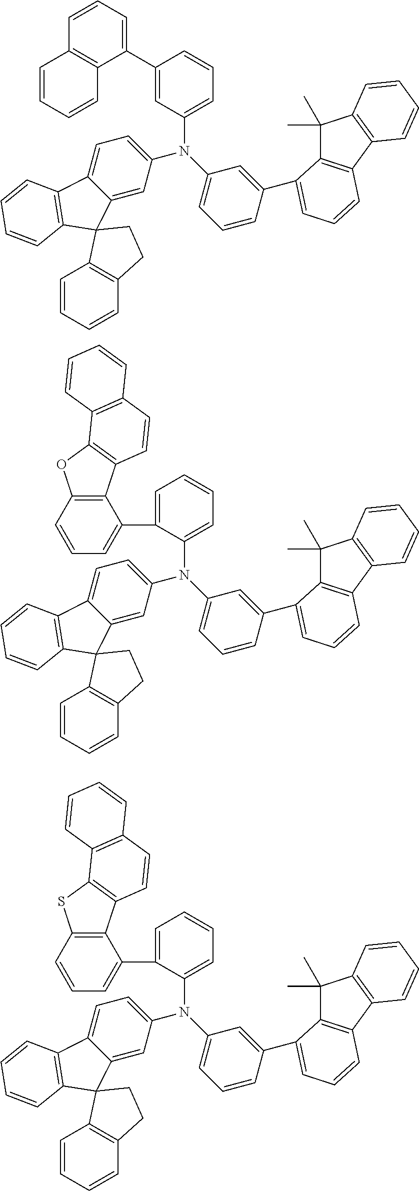

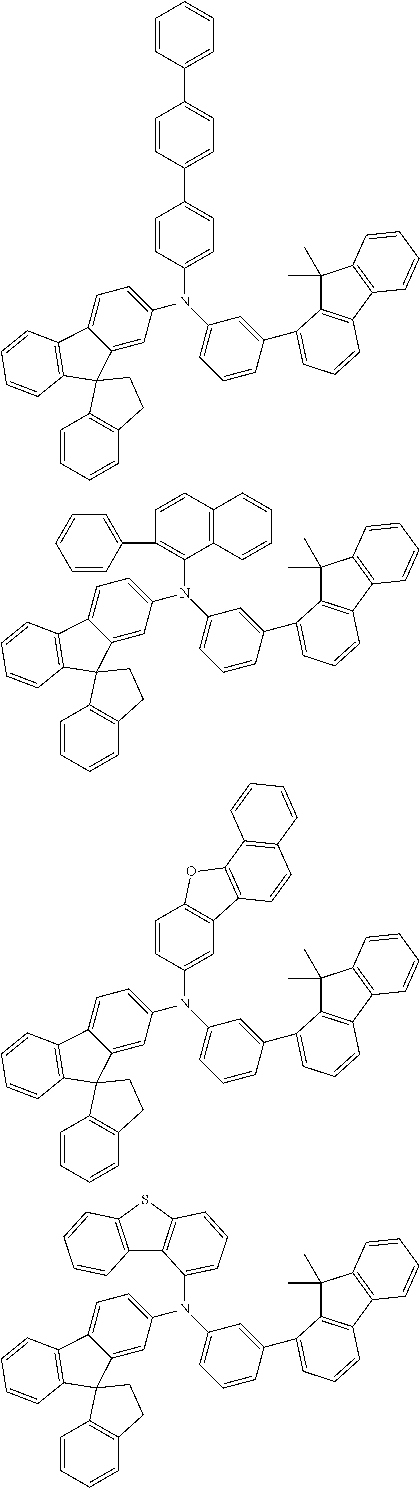

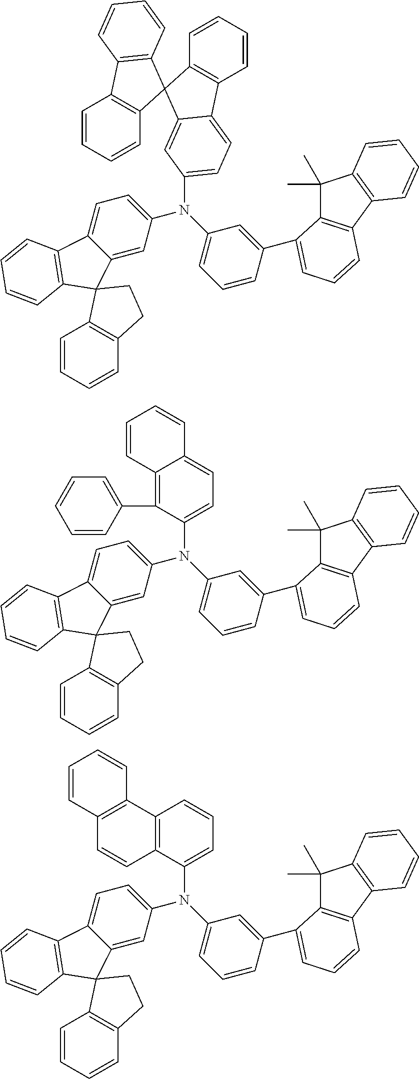

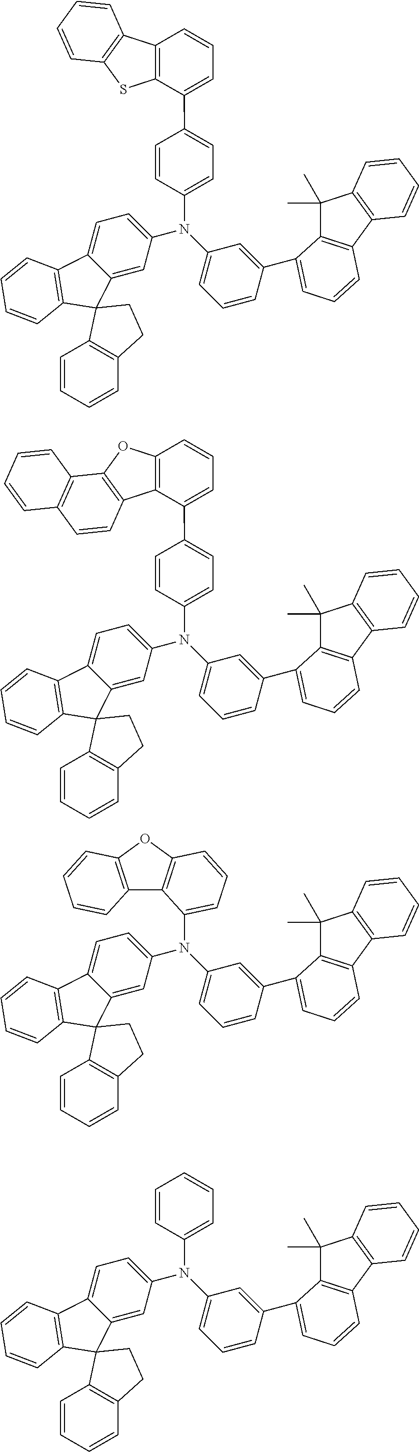

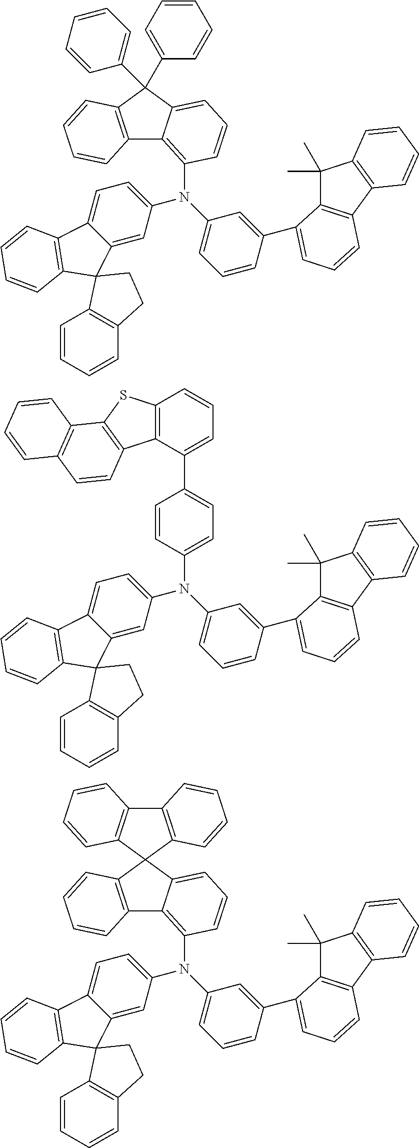

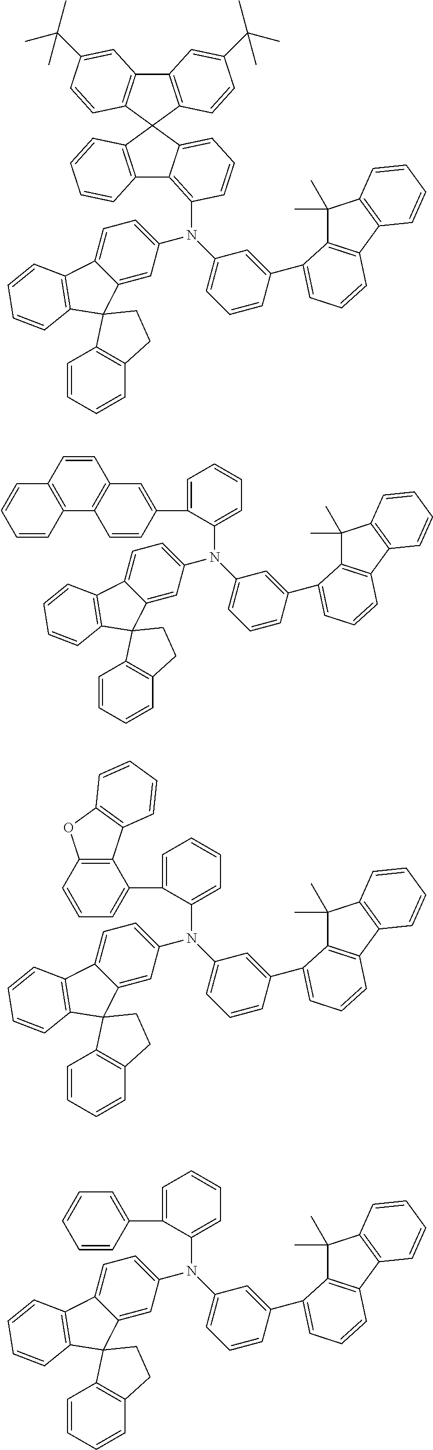

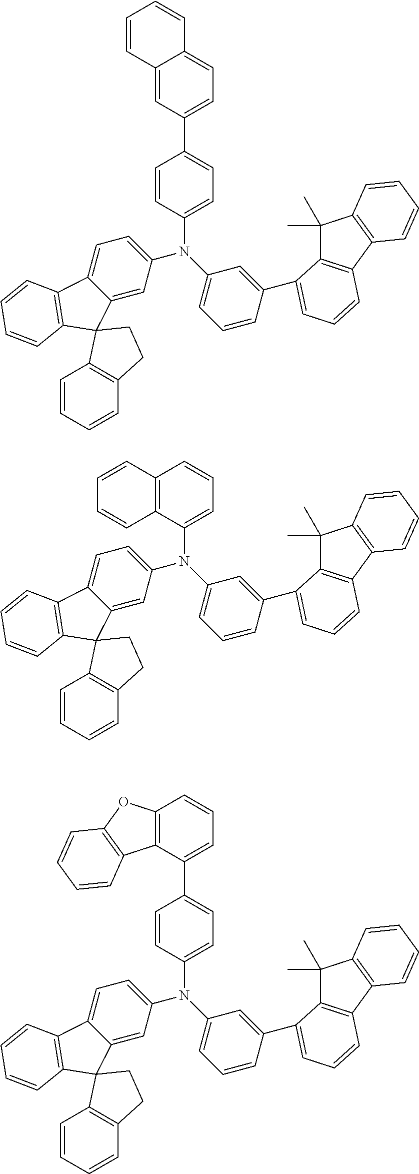 [SMILES: CC1(C)c2ccccc2-c2cccc(-c3cccc(N(c4ccc(-c5ccc6ccccc6c5)cc4)c4ccc5c(c4)C4(CCc6ccccc64)c4ccccc4-5)c3)c21 ; CC1(C)c2ccccc2-c2cccc(-c3cccc(N(c4ccc5c(c4)C4(CCc6ccccc64)c4ccccc4-5)c4cccc5ccccc45)c3)c21 ; CC1(C)c2ccccc2-c2cccc(-c3cccc(N(c4ccc(-c5cccc6oc7ccccc7c56)cc4)c4ccc5c(c4)C4(CCc6ccccc64)c4ccccc4-5)c3)c21]

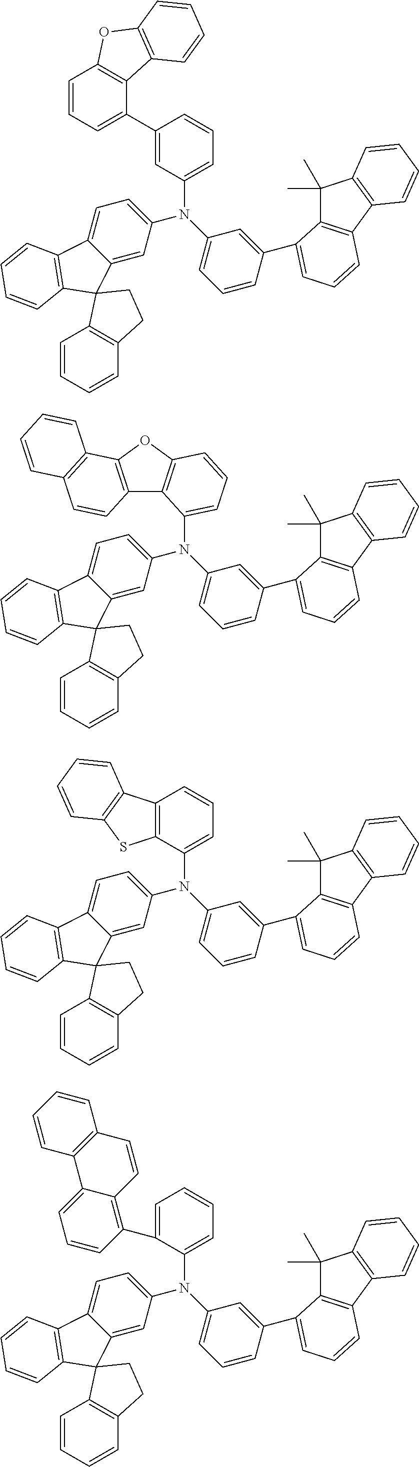

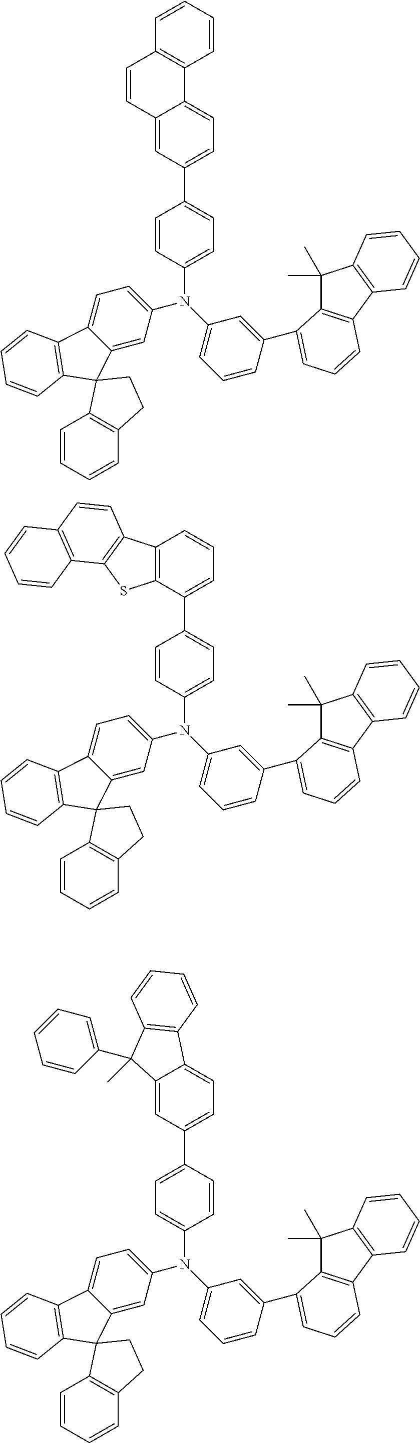

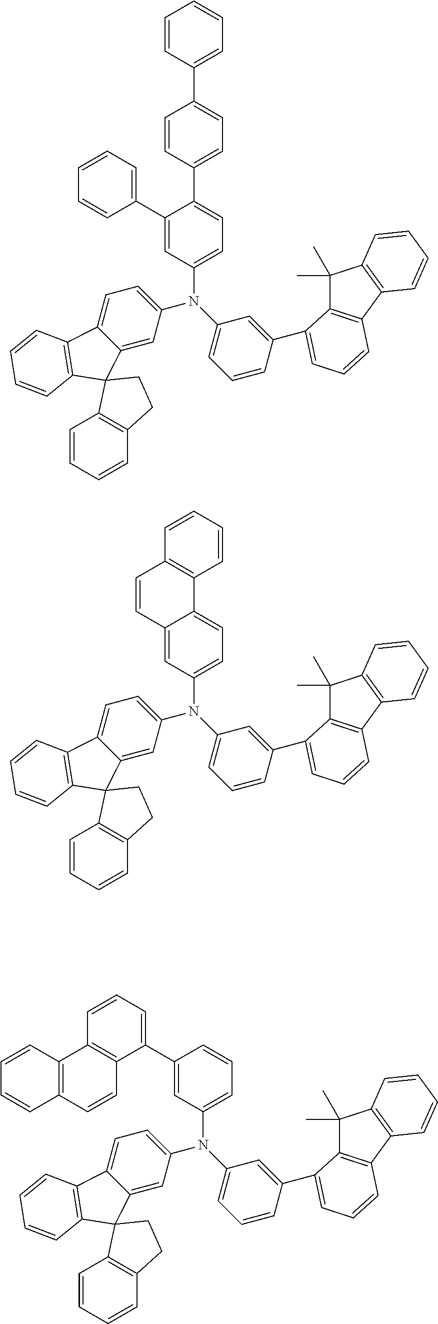 [SMILES: CC1(C)c2ccccc2-c2cccc(-c3cccc(N(c4ccc(-c5ccc(-c6ccccc6)cc5)c(-c5ccccc5)c4)c4ccc5c(c4)C4(CCc6ccccc64)c4ccccc4-5)c3)c21 ; CC1(C)c2ccccc2-c2cccc(-c3cccc(N(c4ccc5c(c4)C4(CCc6ccccc64)c4ccccc4-5)c4ccc5c(ccc6ccccc65)c4)c3)c21 ; CC1(C)c2ccccc2-c2cccc(-c3cccc(N(c4cccc(-c5cccc6c5ccc5ccccc56)c4)c4ccc5c(c4)C4(CCc6ccccc64)c4ccccc4-5)c3)c21]

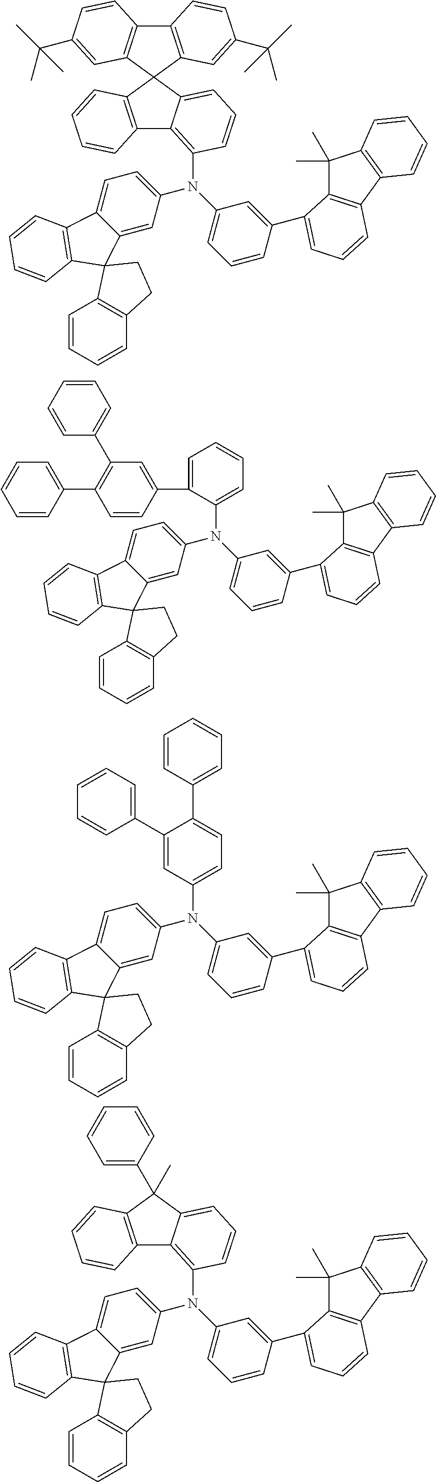

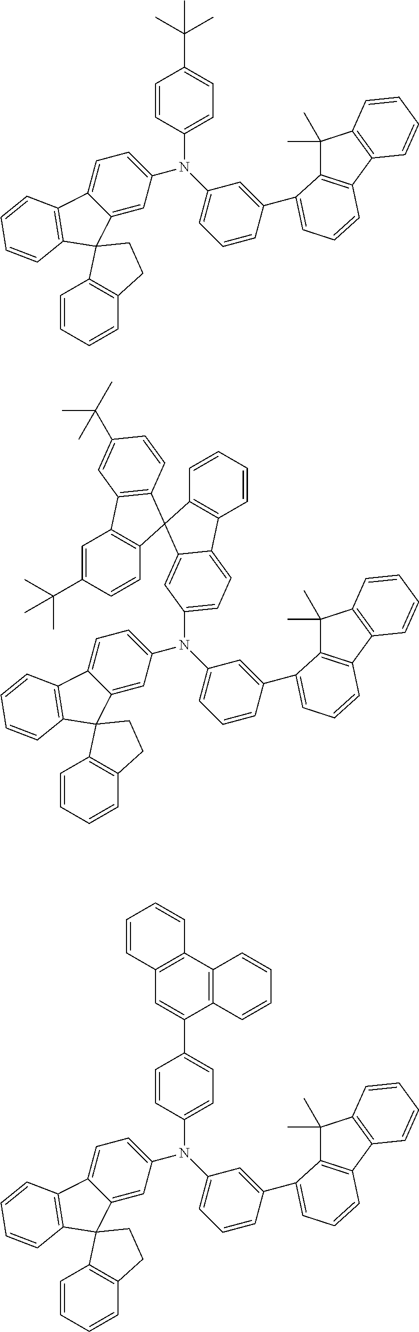

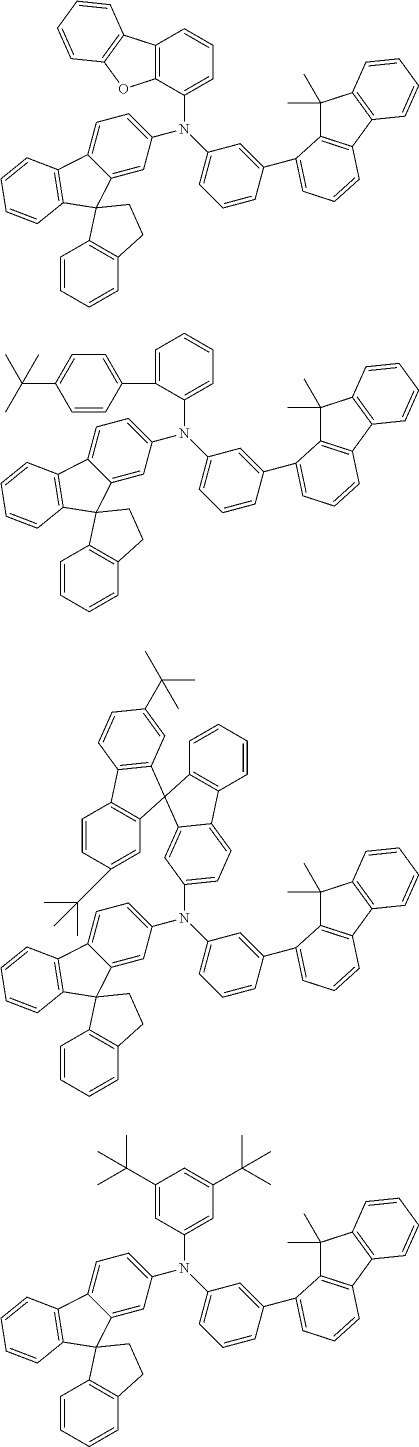 [SMILES: CC1(C)c2ccccc2-c2cccc(-c3cccc(N(c4ccc5c(c4)C4(CCc6ccccc64)c4ccccc4-5)c4cccc5c4oc4ccccc45)c3)c21 ; CC(C)(C)c1ccc(-c2ccccc2N(c2cccc(-c3cccc4c3C(C)(C)c3ccccc3-4)c2)c2ccc3c(c2)C2(CCc4ccccc42)c2ccccc2-3)cc1 ; CC(C)(C)c1ccc2c(c1)C1(c3ccccc3-c3ccc(N(c4cccc(-c5cccc6c5C(C)(C)c5ccccc5-6)c4)c4ccc5c(c4)C4(CCc6ccccc64)c4ccccc4-5)cc31)c1cc(C(C)(C)C)ccc1-2 ; CC(C)(C)c1cc(N(c2cccc(-c3cccc4c3C(C)(C)c3ccccc3-4)c2)c2ccc3c(c2)C2(CCc4ccccc42)c2ccccc2-3)cc(C(C)(C)C)c1]

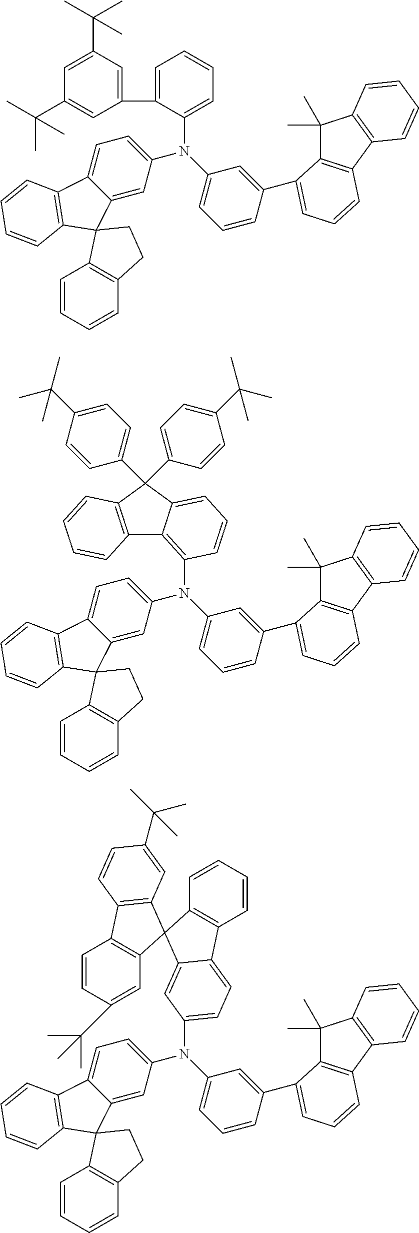

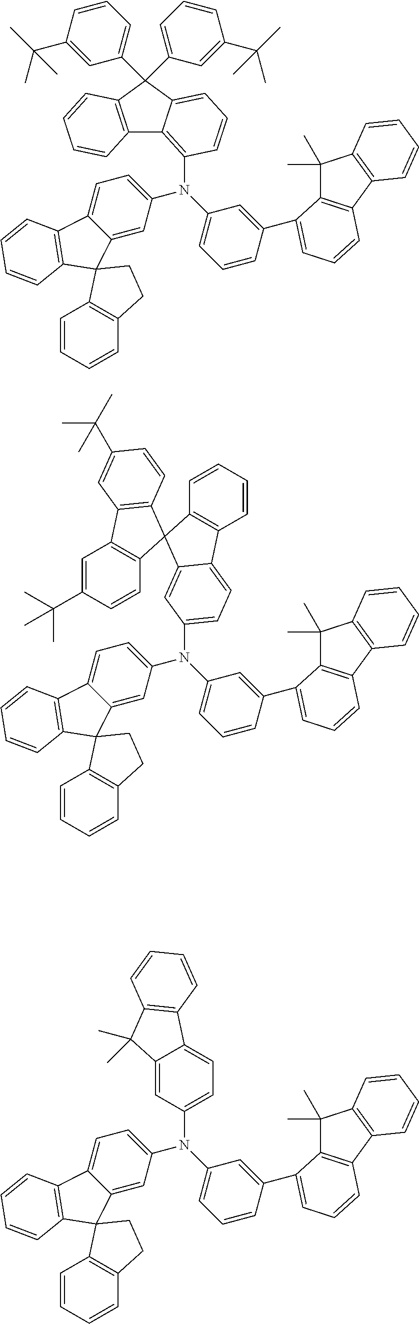

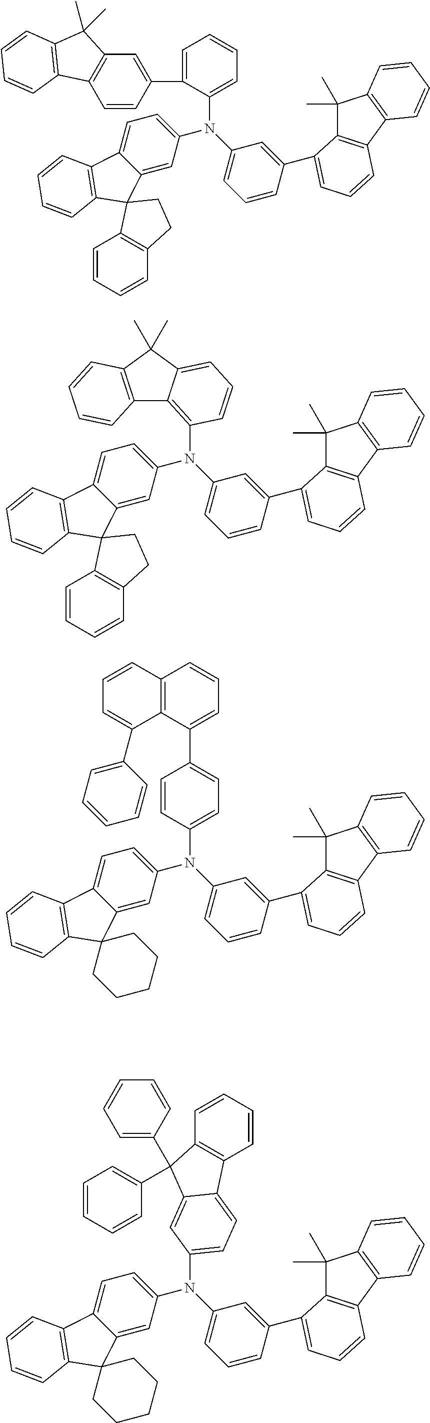

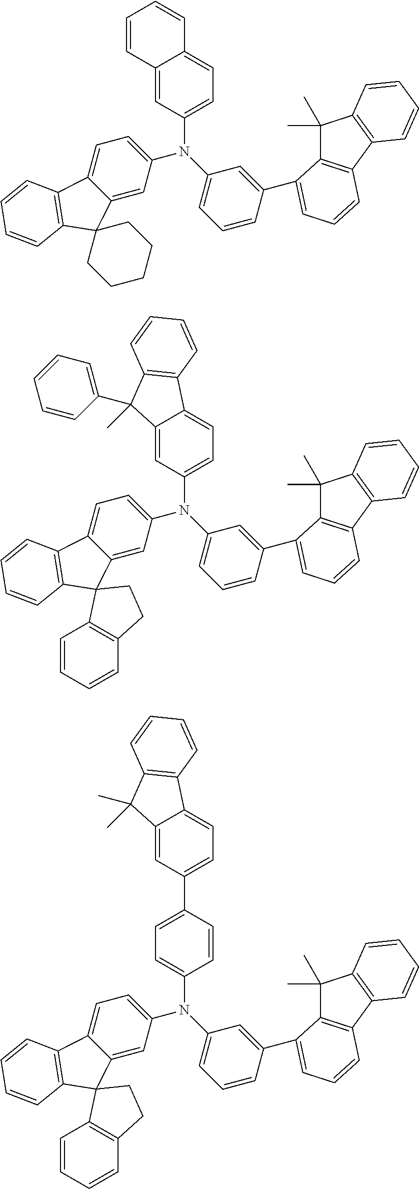

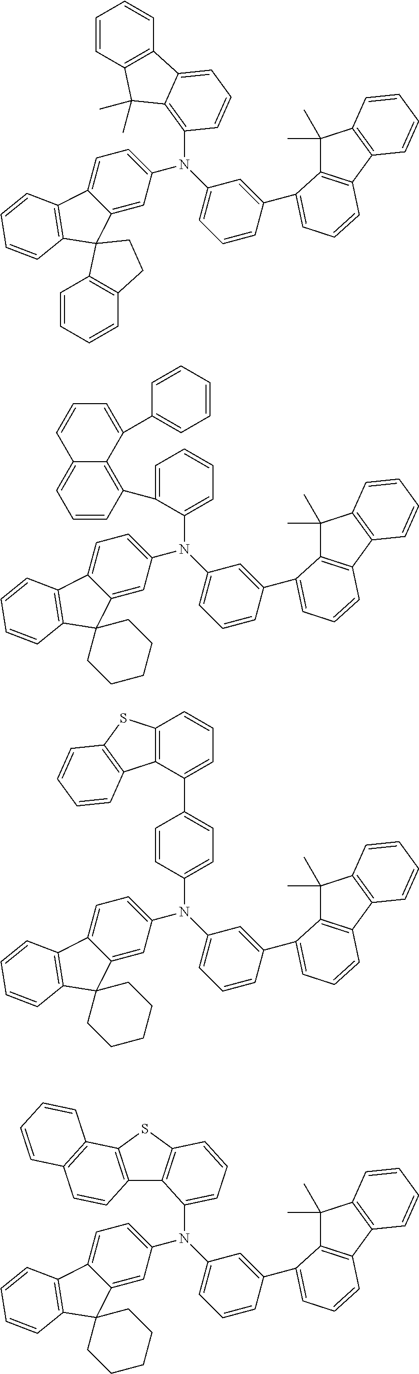

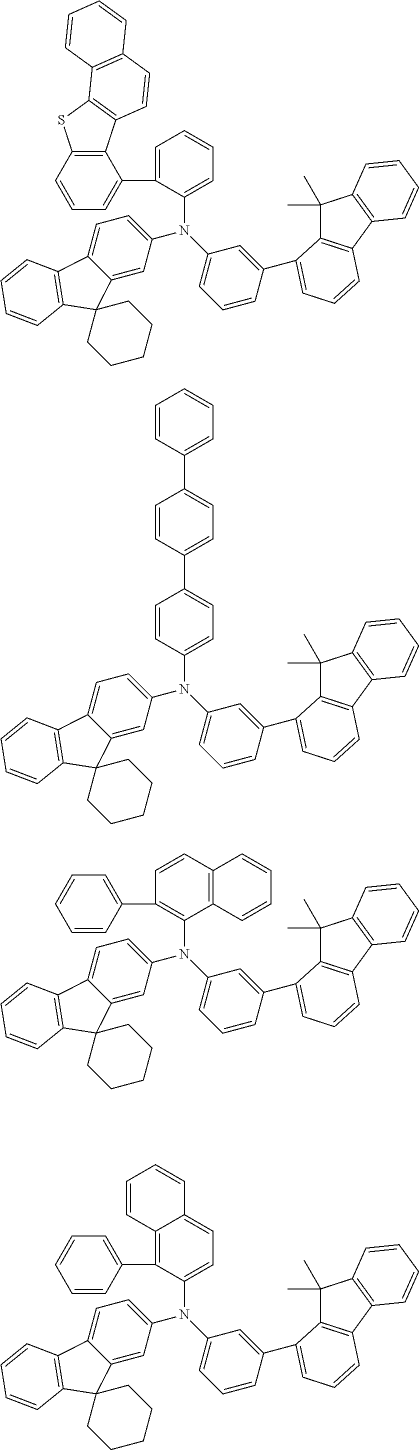 [SMILES: CC1(C)c2ccccc2-c2cccc(-c3cccc(N(c4ccc5c(c4)C4(CCCCC4)c4ccccc4-5)c4ccccc4-c4cccc5sc6c7ccccc7ccc6c45)c3)c21 ; CC1(C)c2ccccc2-c2cccc(-c3cccc(N(c4ccc(-c5ccc(-c6ccccc6)cc5)cc4)c4ccc5c(c4)C4(CCCCC4)c4ccccc4-5)c3)c21 ; CC1(C)c2ccccc2-c2cccc(-c3cccc(N(c4ccc5c(c4)C4(CCCCC4)c4ccccc4-5)c4c(-c5ccccc5)ccc5ccccc45)c3)c21 ; CC1(C)c2ccccc2-c2cccc(-c3cccc(N(c4ccc5c(c4)C4(CCCCC4)c4ccccc4-5)c4ccc5ccccc5c4-c4ccccc4)c3)c21]

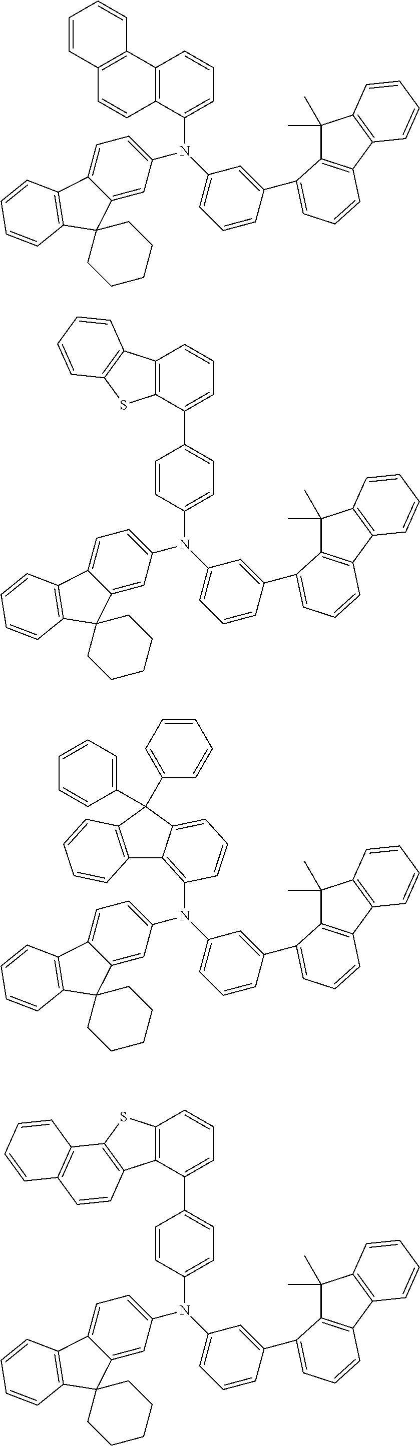

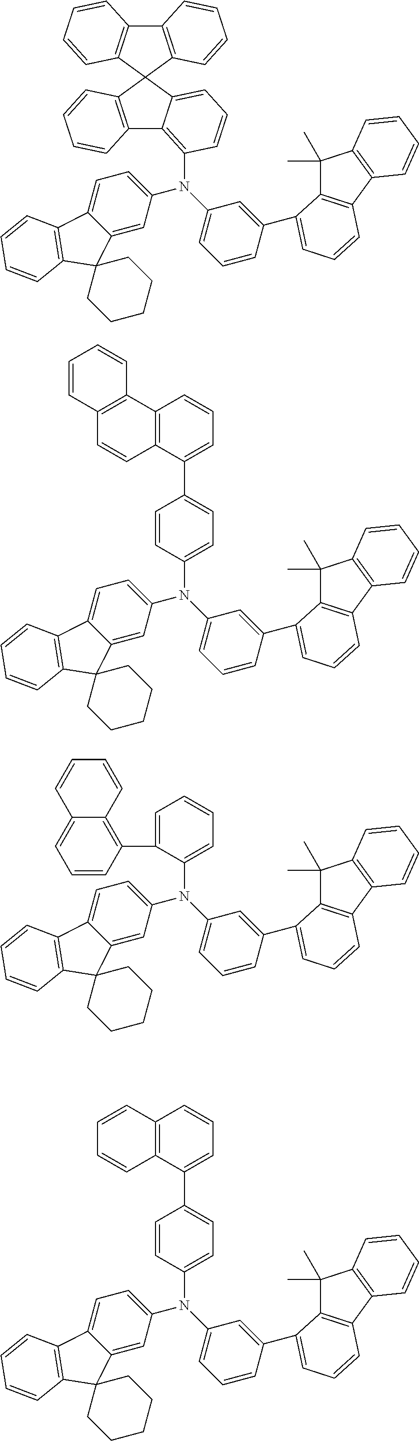

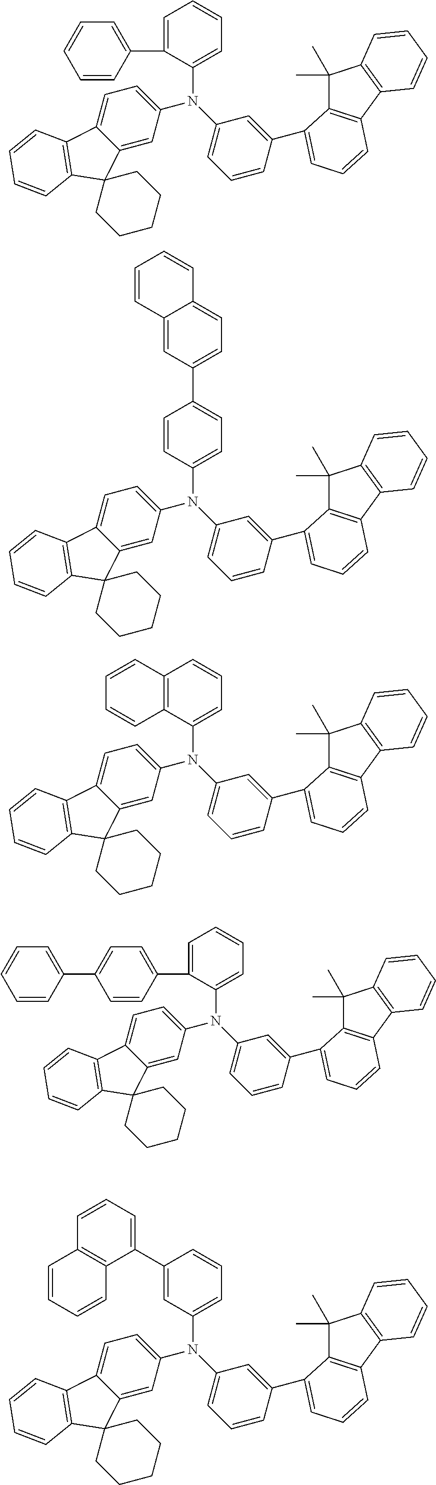

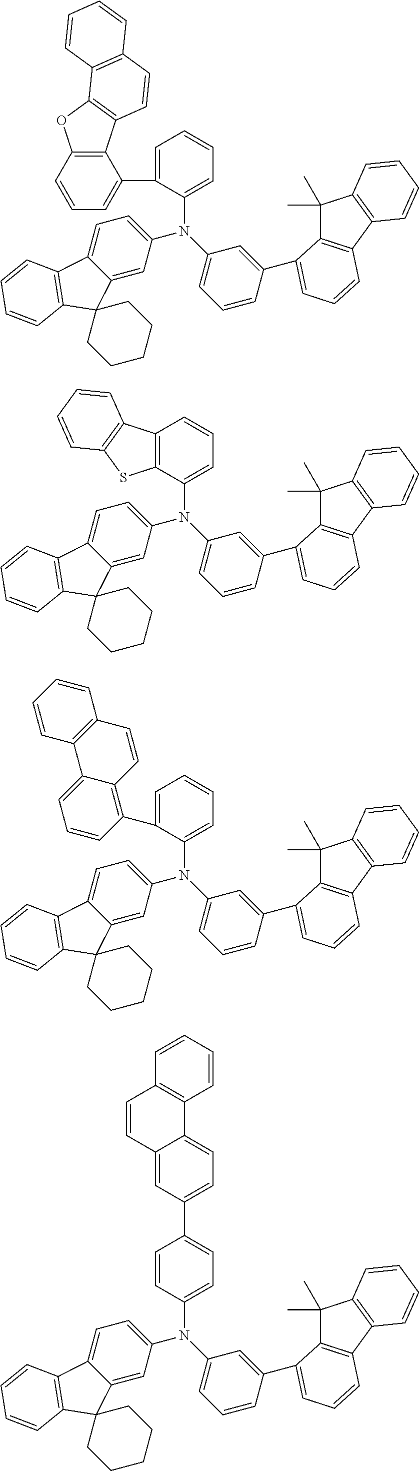

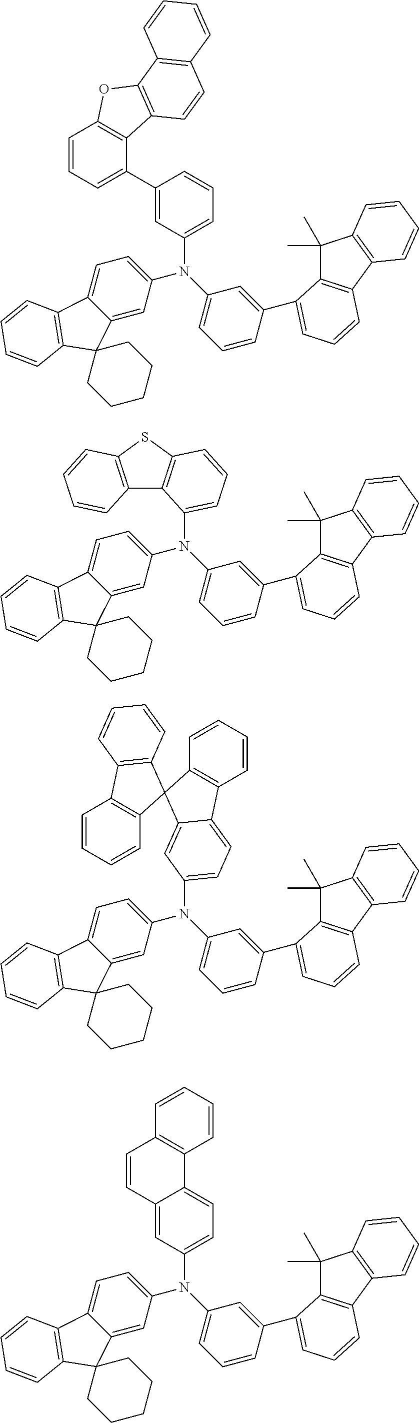

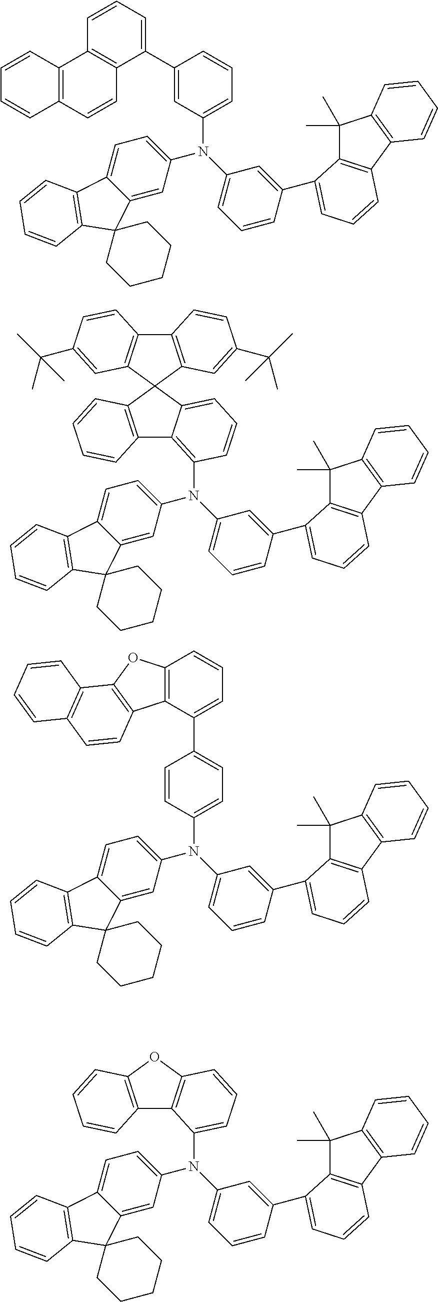

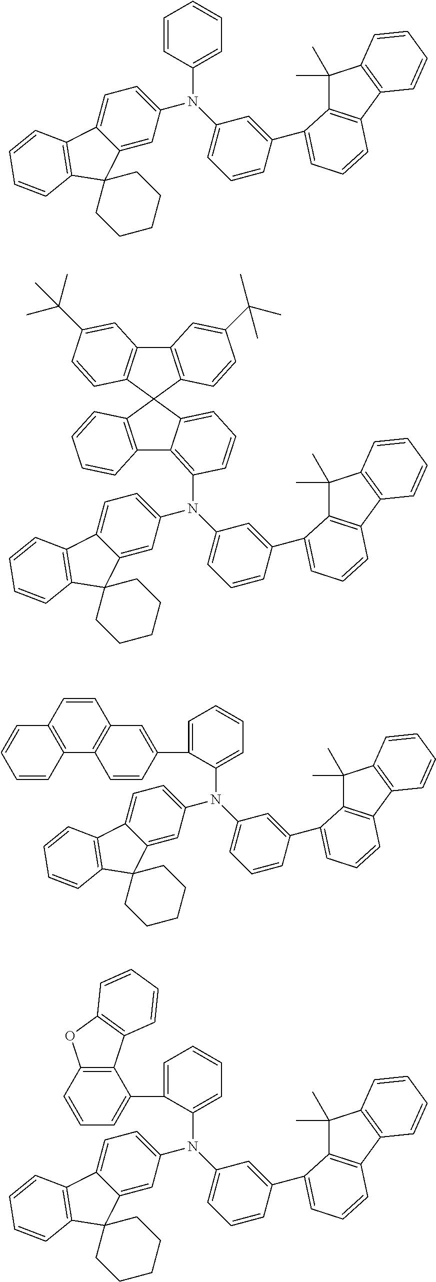

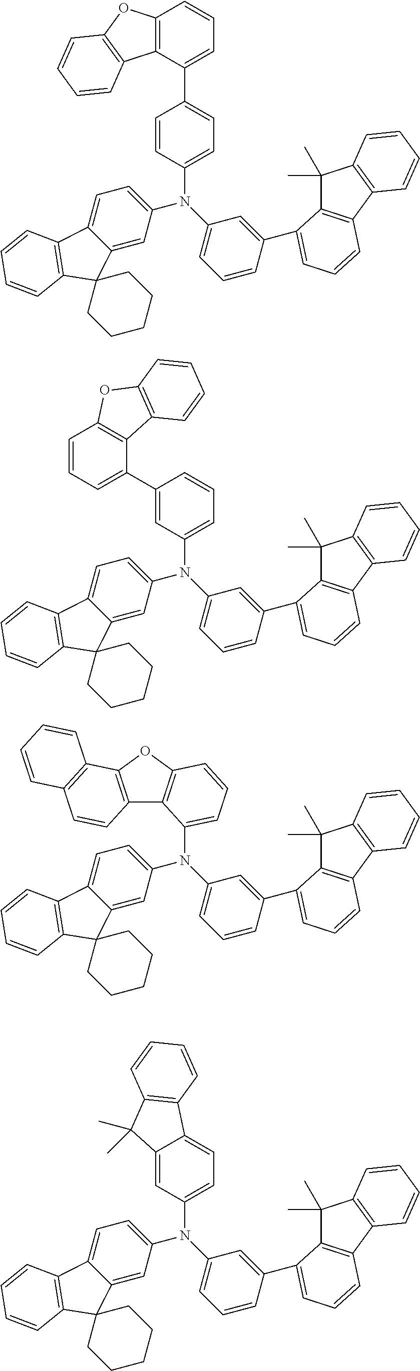

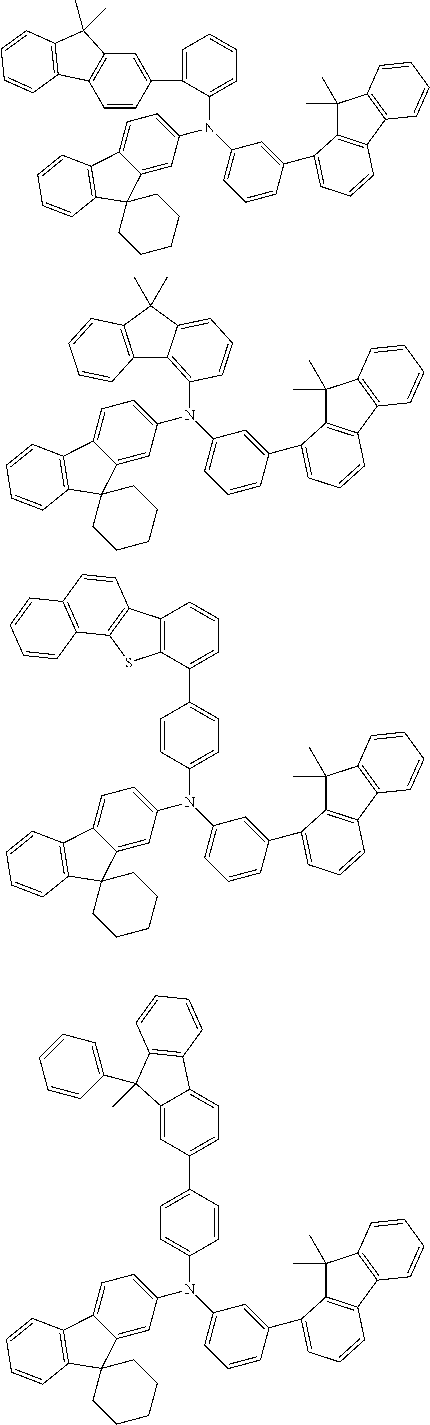

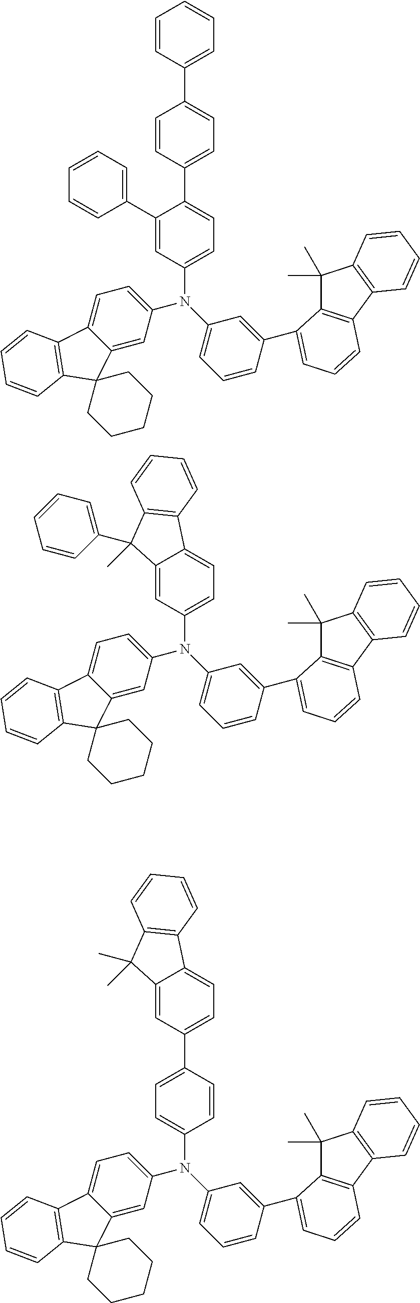

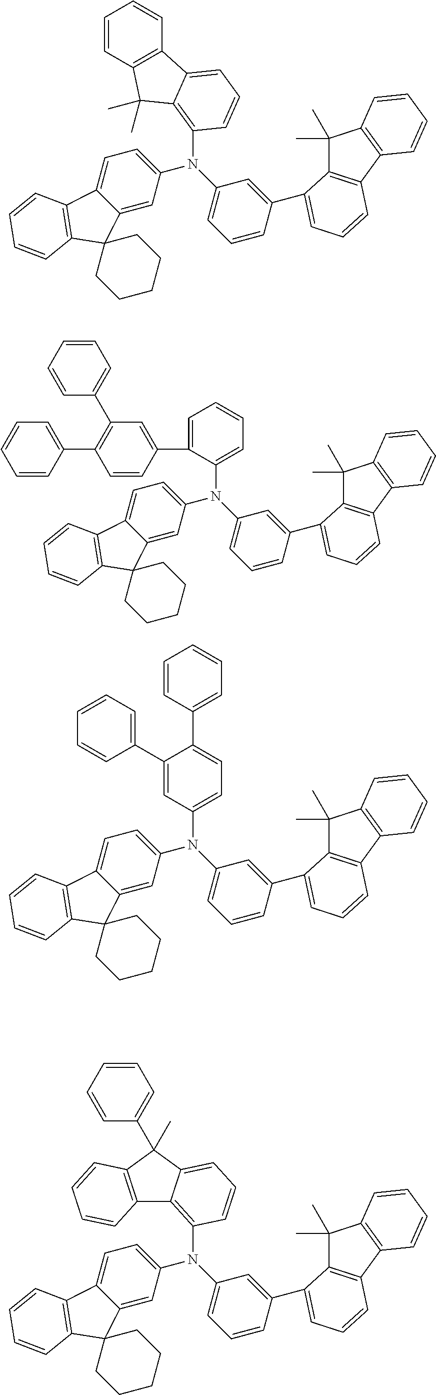

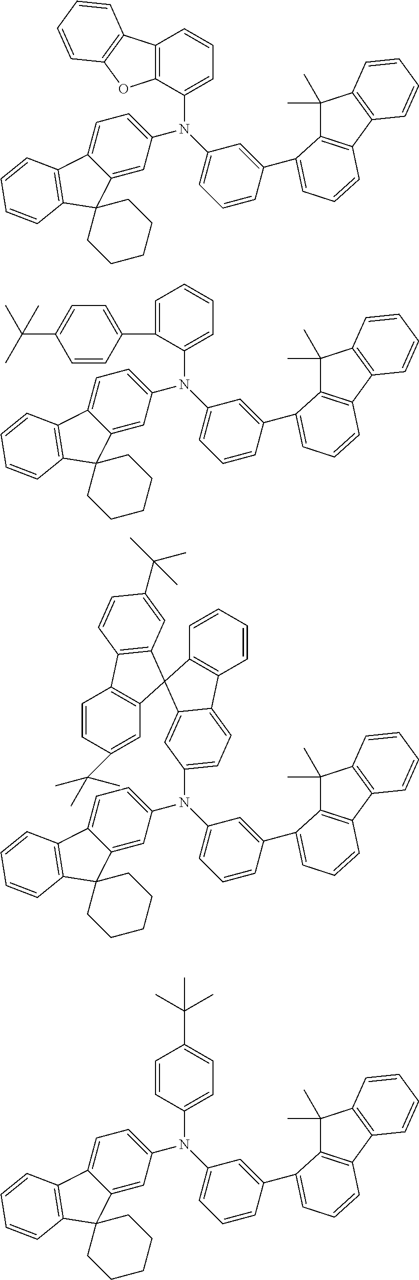

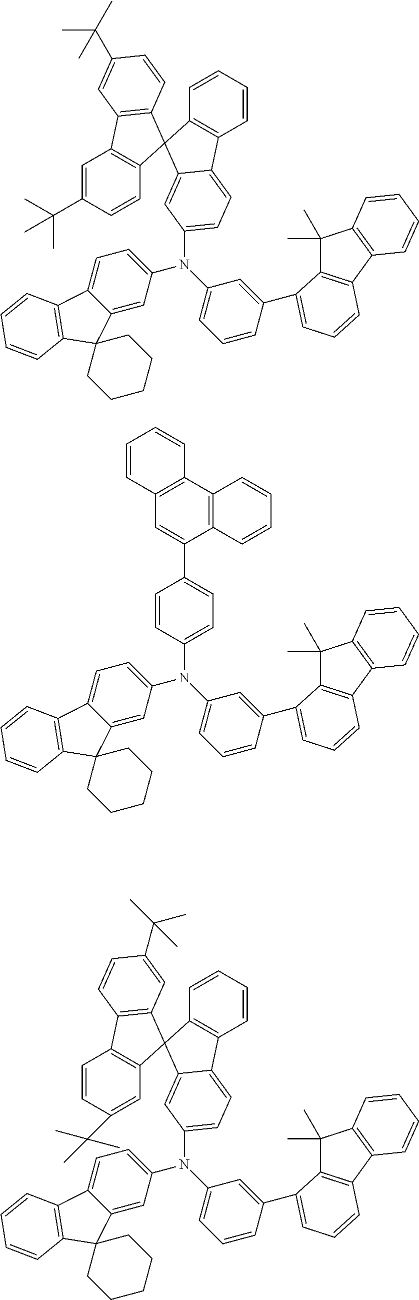

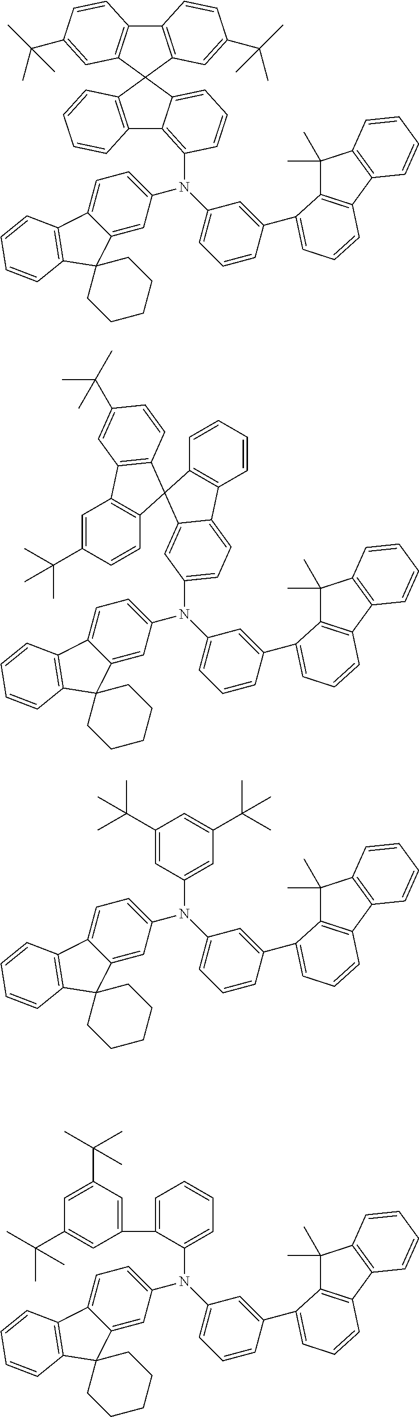

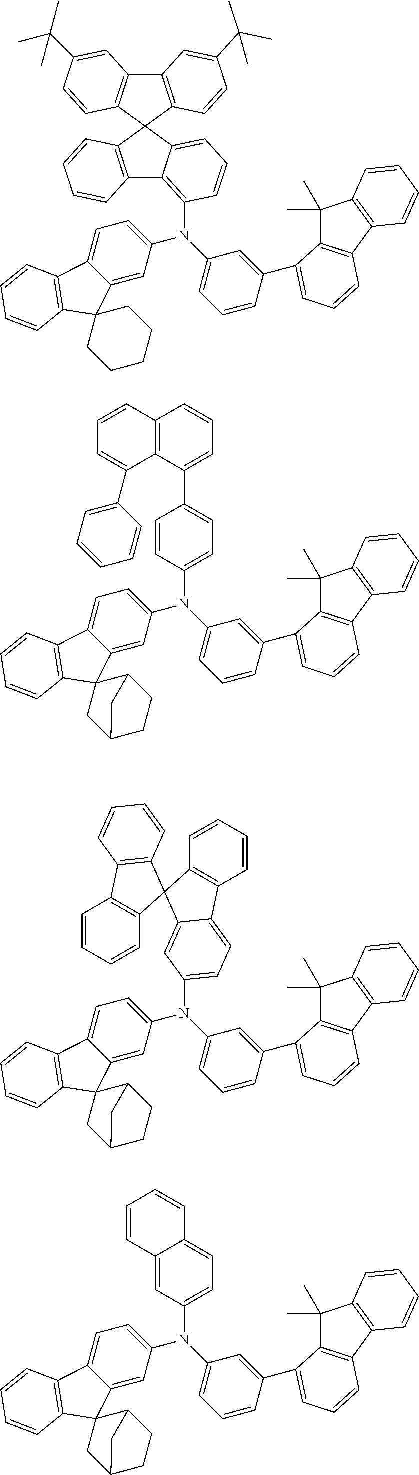

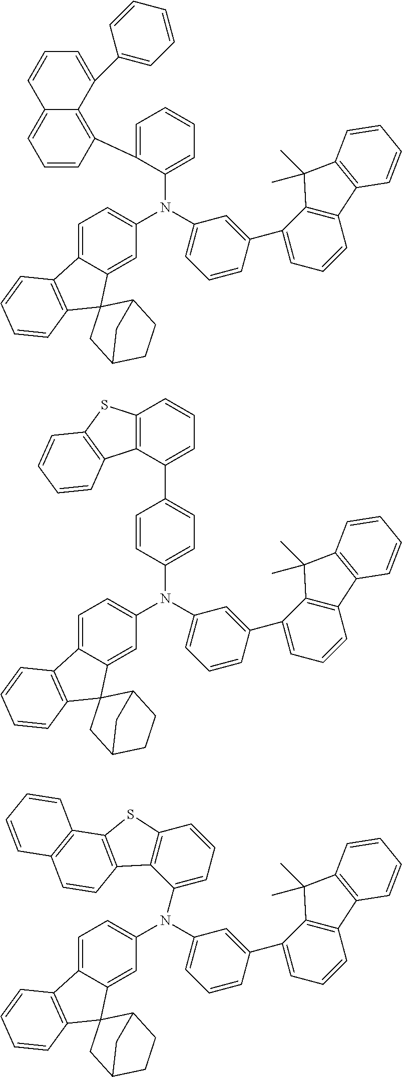

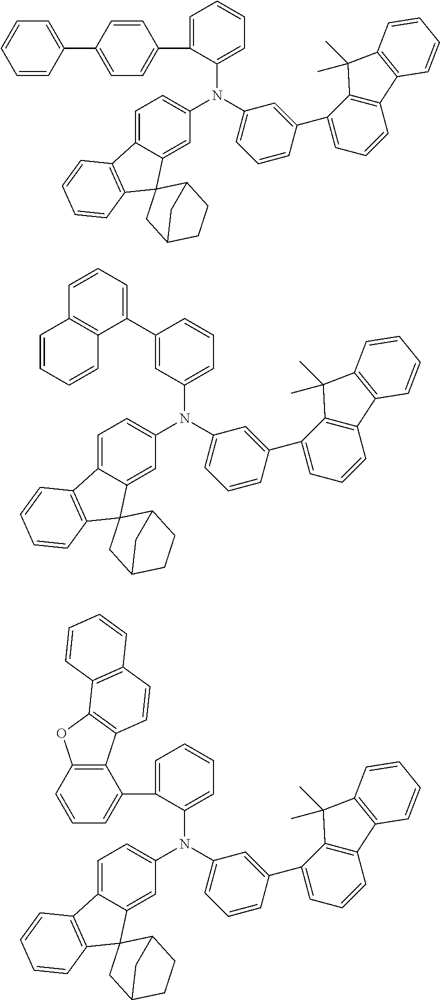 [SMILES: CC1(C)c2ccccc2-c2cccc(-c3cccc(N(c4ccc5c(c4)C4(CC6CCC4C6)c4ccccc4-5)c4ccccc4-c4ccc(-c5ccccc5)cc4)c3)c21 ; CC1(C)c2ccccc2-c2cccc(-c3cccc(N(c4cccc(-c5cccc6ccccc56)c4)c4ccc5c(c4)C4(CC6CCC4C6)c4ccccc4-5)c3)c21 ; CC1(C)c2ccccc2-c2cccc(-c3cccc(N(c4ccc5c(c4)C4(CC6CCC4C6)c4ccccc4-5)c4ccccc4-c4cccc5oc6c7ccccc7ccc6c45)c3)c21]

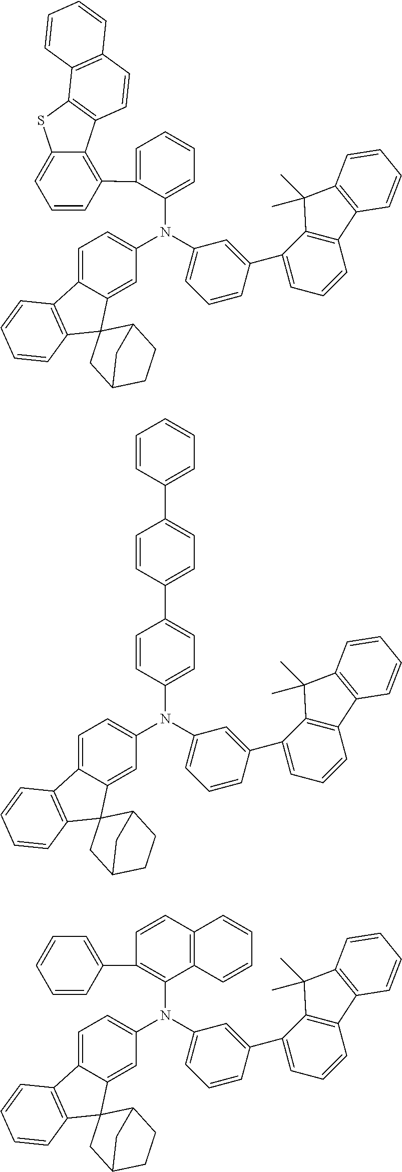

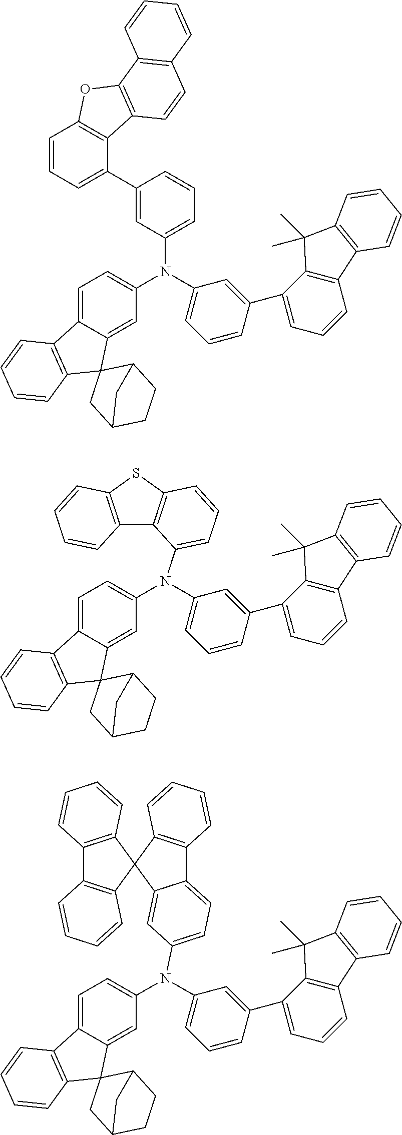 [SMILES: CC1(C)c2ccccc2-c2cccc(-c3cccc(N(c4cccc(-c5cccc6oc7c8ccccc8ccc7c56)c4)c4ccc5c(c4)C4(CC6CCC4C6)c4ccccc4-5)c3)c21 ; CC1(C)c2ccccc2-c2cccc(-c3cccc(N(c4ccc5c(c4)C4(CC6CCC4C6)c4ccccc4-5)c4cccc5sc6ccccc6c45)c3)c21 ; CC1(C)c2ccccc2-c2cccc(-c3cccc(N(c4ccc5c(c4)C4(c6ccccc6-c6ccccc64)c4ccccc4-5)c4ccc5c(c4)C4(CC6CCC4C6)c4ccccc4-5)c3)c21]

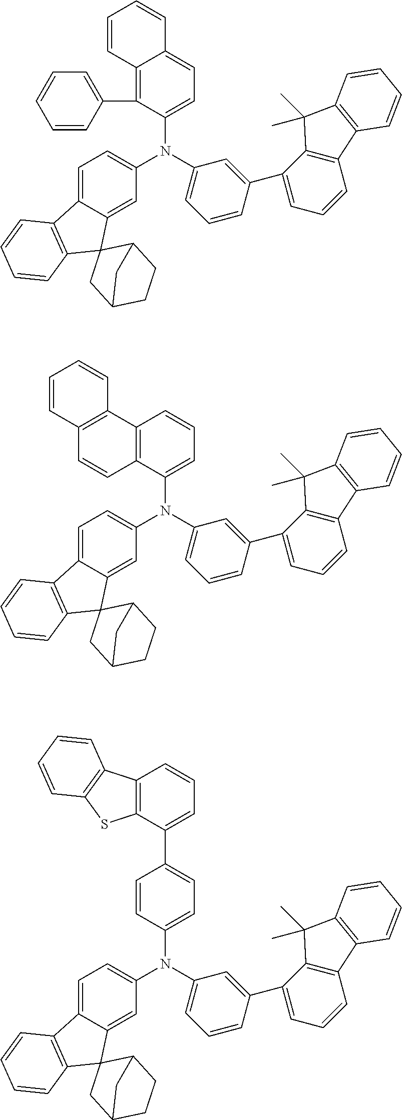 [SMILES: CC1(C)c2ccccc2-c2cccc(-c3cccc(N(c4ccc5c(c4)C4(CC6CCC4C6)c4ccccc4-5)c4ccc5ccccc5c4-c4ccccc4)c3)c21 ; CC1(C)c2ccccc2-c2cccc(-c3cccc(N(c4ccc5c(c4)C4(CC6CCC4C6)c4ccccc4-5)c4cccc5c4ccc4ccccc45)c3)c21 ; CC1(C)c2ccccc2-c2cccc(-c3cccc(N(c4ccc(-c5cccc6c5sc5ccccc56)cc4)c4ccc5c(c4)C4(CC6CCC4C6)c4ccccc4-5)c3)c21]

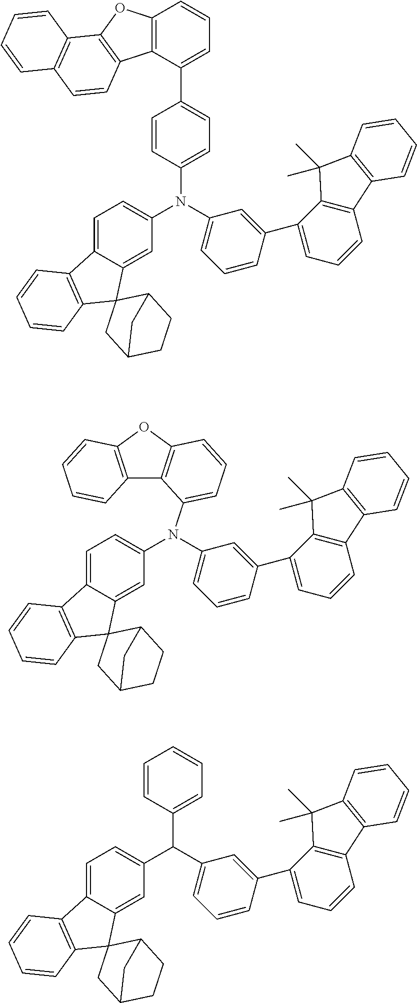

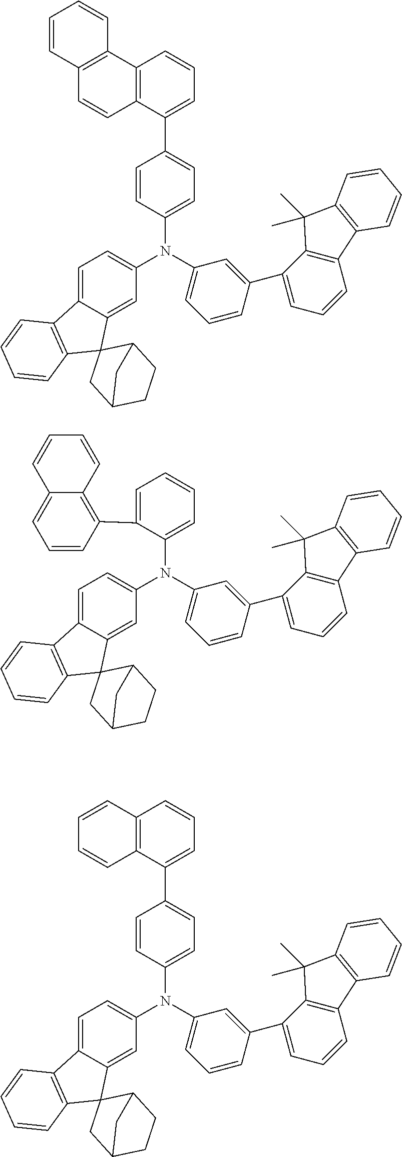

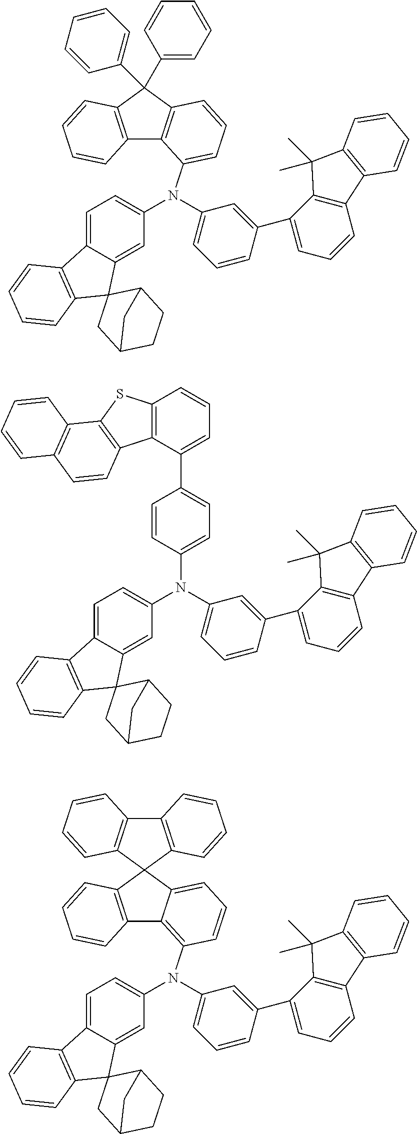

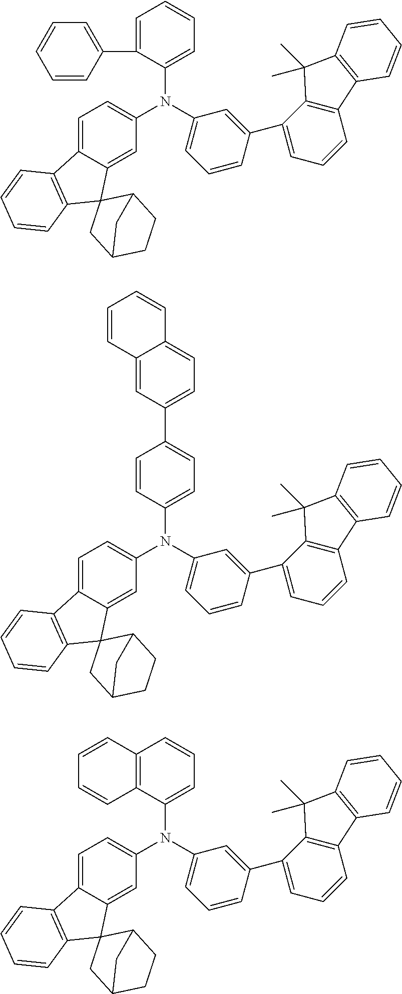

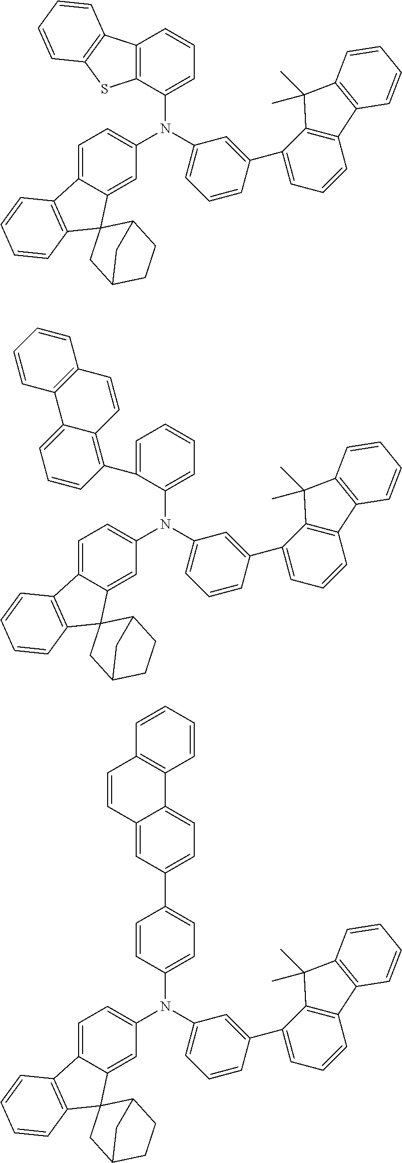

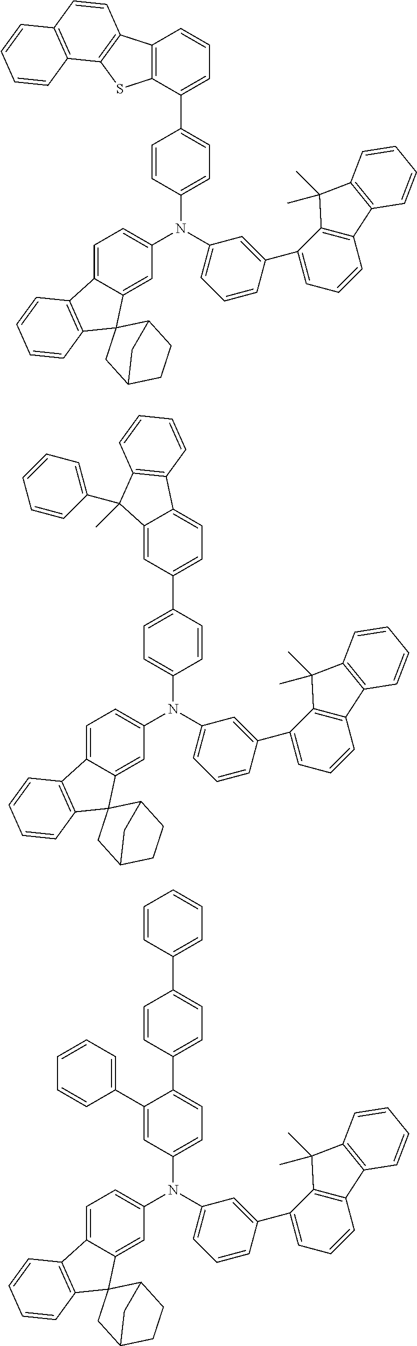

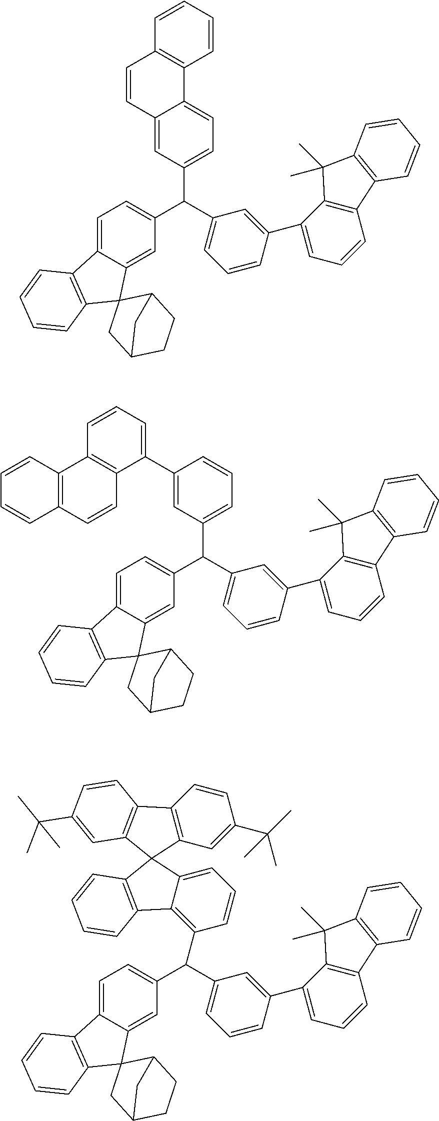

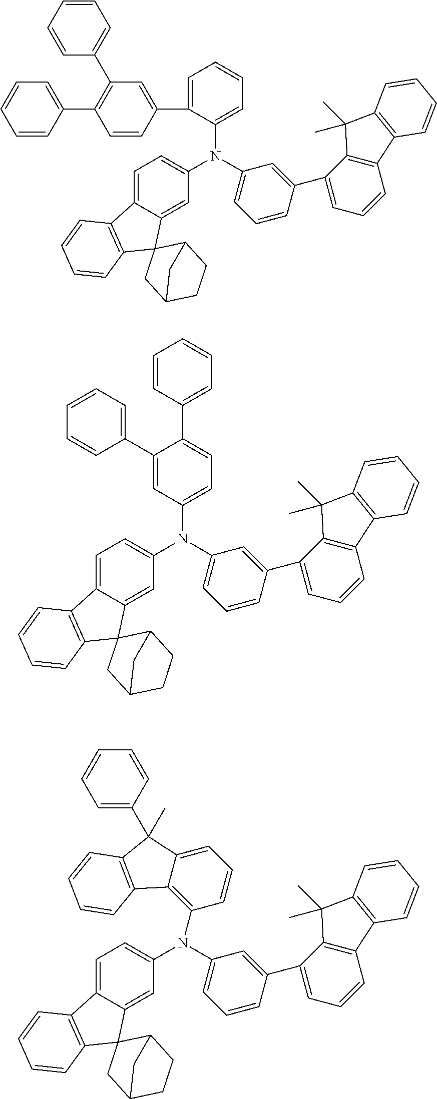

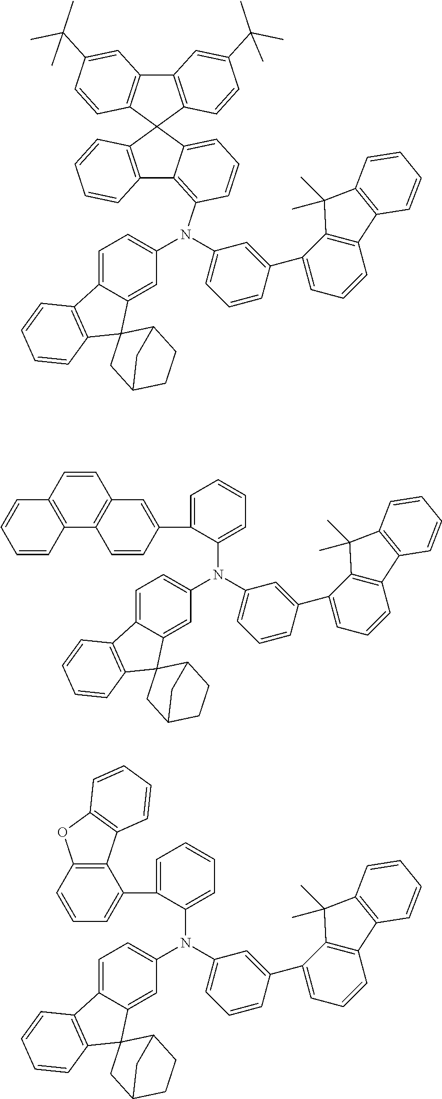 [SMILES: CC(C)(C)c1ccc2c(c1)-c1cc(C(C)(C)C)ccc1C21c2ccccc2-c2c(N(c3cccc(-c4cccc5c4C(C)(C)c4ccccc4-5)c3)c3ccc4c(c3)C3(CC5CCC3C5)c3ccccc3-4)cccc21 ; CC1(C)c2ccccc2-c2cccc(-c3cccc(N(c4ccc5c(c4)C4(CC6CCC4C6)c4ccccc4-5)c4ccccc4-c4ccc5c(ccc6ccccc65)c4)c3)c21 ; CC1(C)c2ccccc2-c2cccc(-c3cccc(N(c4ccc5c(c4)C4(CC6CCC4C6)c4ccccc4-5)c4ccccc4-c4cccc5oc6ccccc6c45)c3)c21]

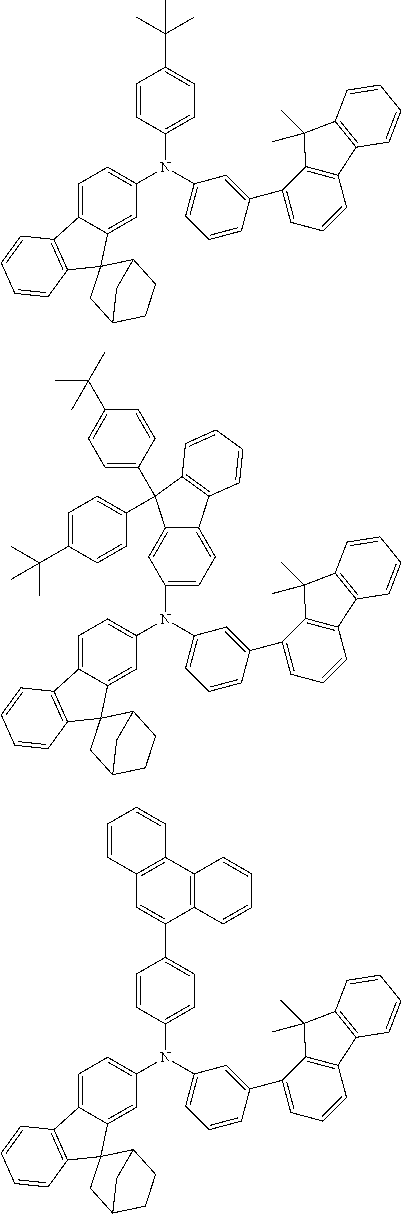

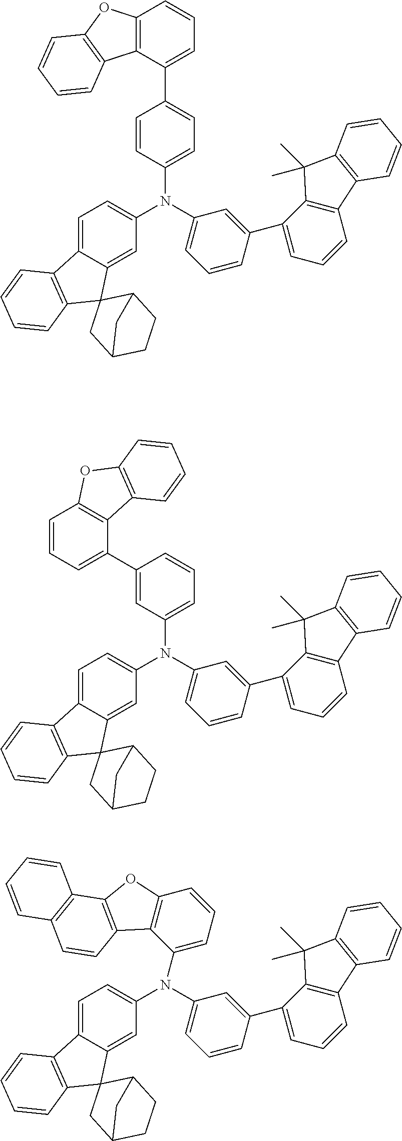 [SMILES: CC1(C)c2ccccc2-c2cccc(-c3cccc(N(c4ccc(-c5cccc6oc7ccccc7c56)cc4)c4ccc5c(c4)C4(CC6CCC4C6)c4ccccc4-5)c3)c21 ; CC1(C)c2ccccc2-c2cccc(-c3cccc(N(c4cccc(-c5cccc6oc7ccccc7c56)c4)c4ccc5c(c4)C4(CC6CCC4C6)c4ccccc4-5)c3)c21 ; CC1(C)c2ccccc2-c2cccc(-c3cccc(N(c4ccc5c(c4)C4(CC6CCC4C6)c4ccccc4-5)c4cccc5oc6c7ccccc7ccc6c45)c3)c21]

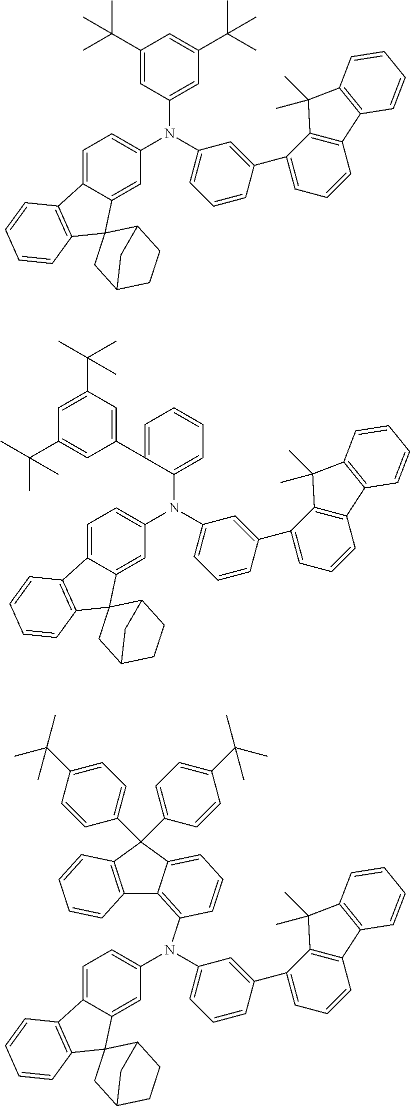 [SMILES: CC(C)(C)c1cc(N(c2cccc(-c3cccc4c3C(C)(C)c3ccccc3-4)c2)c2ccc3c(c2)C2(CC4CCC2C4)c2ccccc2-3)cc(C(C)(C)C)c1 ; CC(C)(C)c1cc(-c2ccccc2N(c2cccc(-c3cccc4c3C(C)(C)c3ccccc3-4)c2)c2ccc3c(c2)C2(CC4CCC2C4)c2ccccc2-3)cc(C(C)(C)C)c1 ; CC(C)(C)c1ccc(C2(c3ccc(C(C)(C)C)cc3)c3ccccc3-c3c(N(c4cccc(-c5cccc6c5C(C)(C)c5ccccc5-6)c4)c4ccc5c(c4)C4(CC6CCC4C6)c4ccccc4-5)cccc32)cc1]

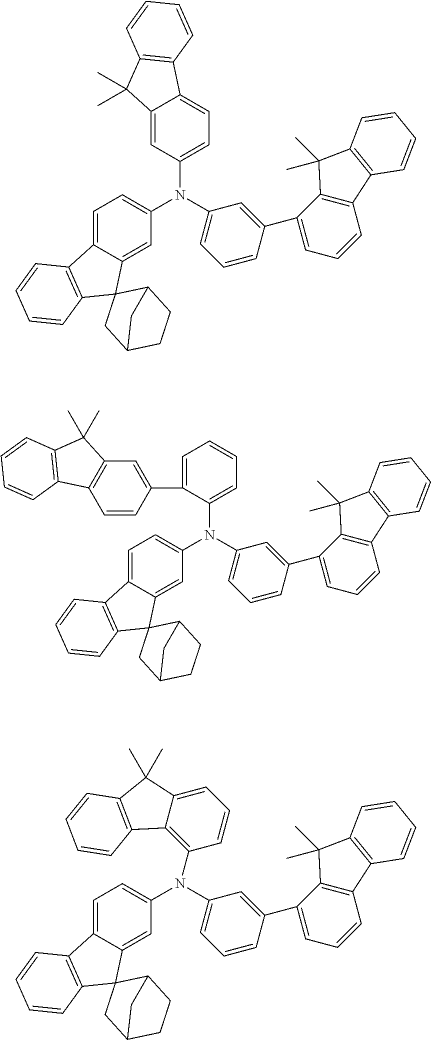 [SMILES: CC1(C)c2ccccc2-c2ccc(N(c3cccc(-c4cccc5c4C(C)(C)c4ccccc4-5)c3)c3ccc4c(c3)C3(CC5CCC3C5)c3ccccc3-4)cc21 ; CC1(C)c2ccccc2-c2ccc(-c3ccccc3N(c3cccc(-c4cccc5c4C(C)(C)c4ccccc4-5)c3)c3ccc4c(c3)C3(CC5CCC3C5)c3ccccc3-4)cc21 ; CC1(C)c2ccccc2-c2c(N(c3cccc(-c4cccc5c4C(C)(C)c4ccccc4-5)c3)c3ccc4c(c3)C3(CC5CCC3C5)c3ccccc3-4)cccc21]

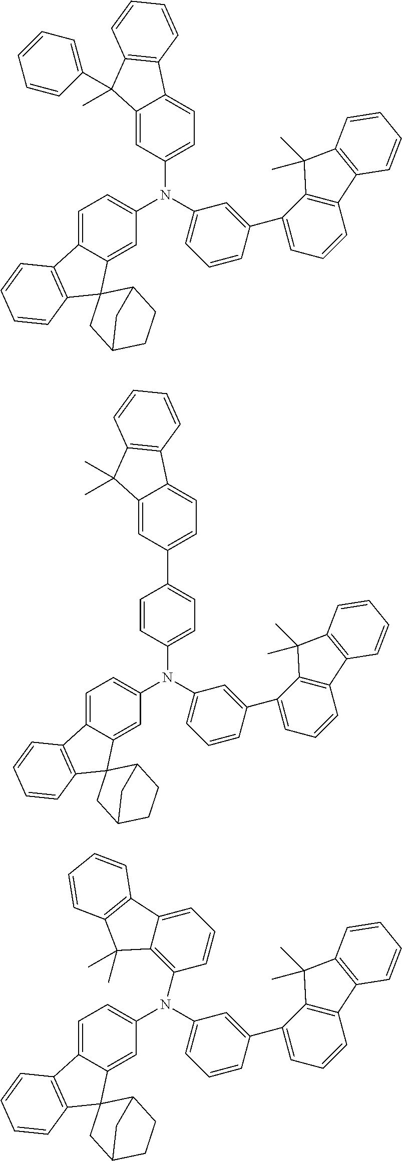

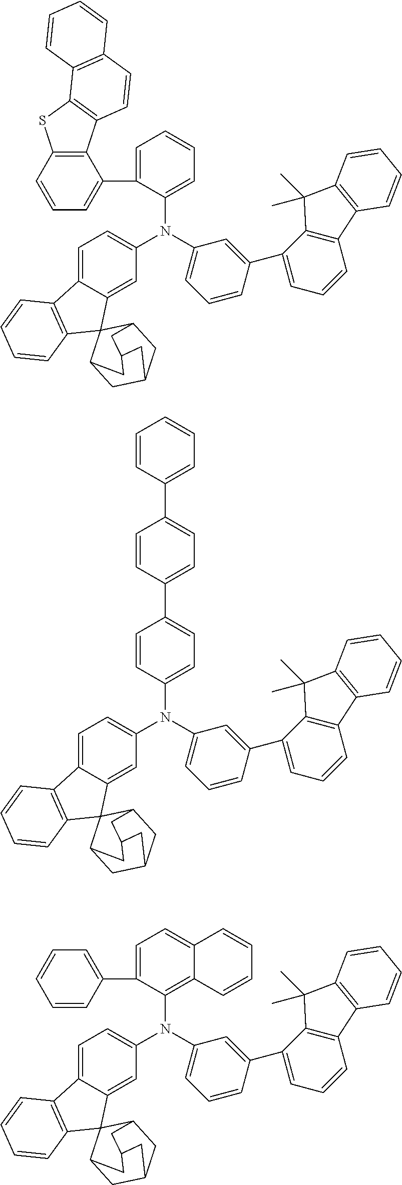

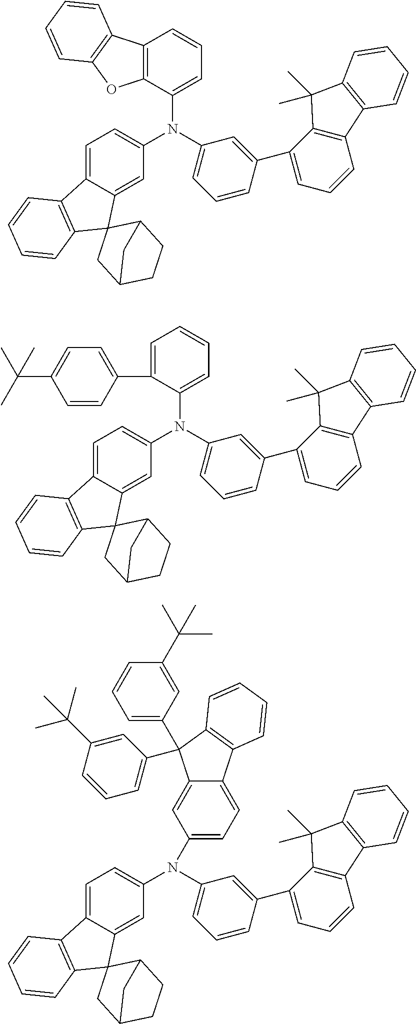

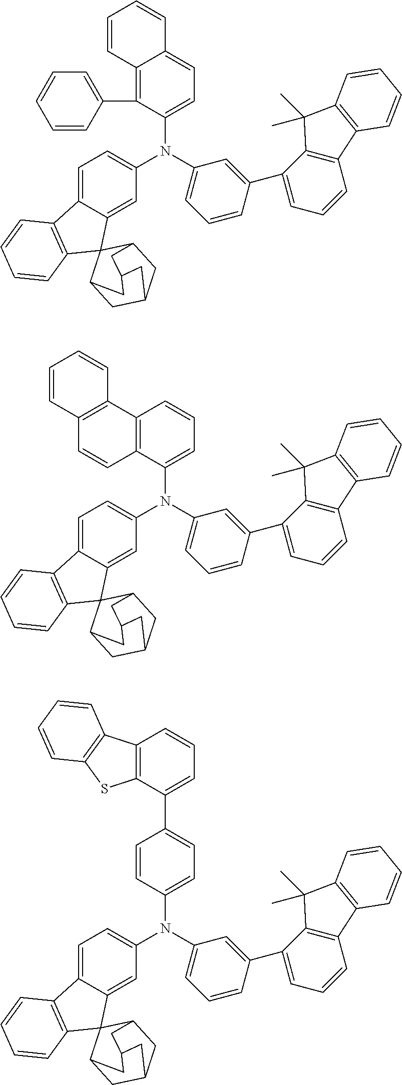 [SMILES: CC1(C)c2ccccc2-c2cccc(-c3cccc(N(c4ccc5c(c4)C4(c6ccccc6-5)C5CC6CC(C5)CC4C6)c4ccc5ccccc5c4-c4ccccc4)c3)c21 ; CC1(C)c2ccccc2-c2cccc(-c3cccc(N(c4ccc5c(c4)C4(c6ccccc6-5)C5CC6CC(C5)CC4C6)c4cccc5c4ccc4ccccc45)c3)c21 ; CC1(C)c2ccccc2-c2cccc(-c3cccc(N(c4ccc(-c5cccc6c5sc5ccccc56)cc4)c4ccc5c(c4)C4(c6ccccc6-5)C5CC6CC(C5)CC4C6)c3)c21]

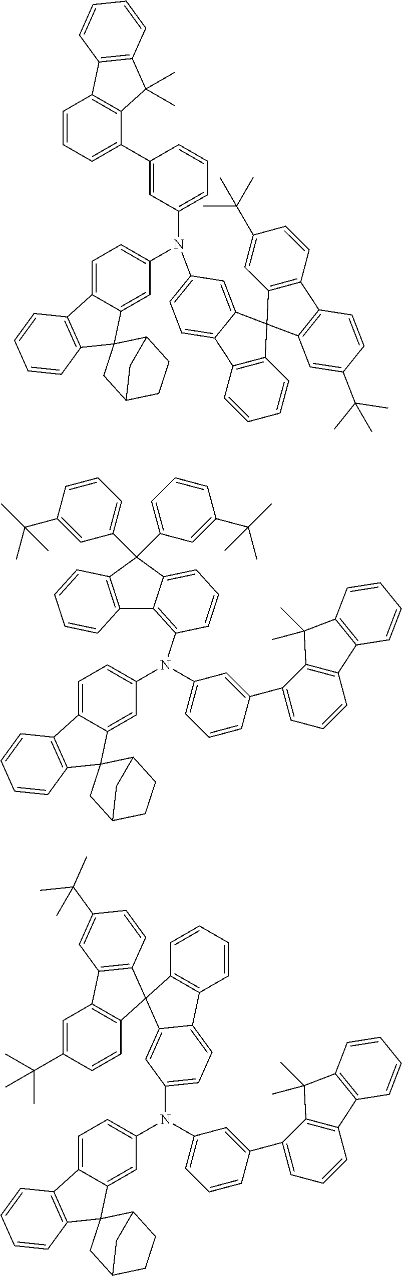

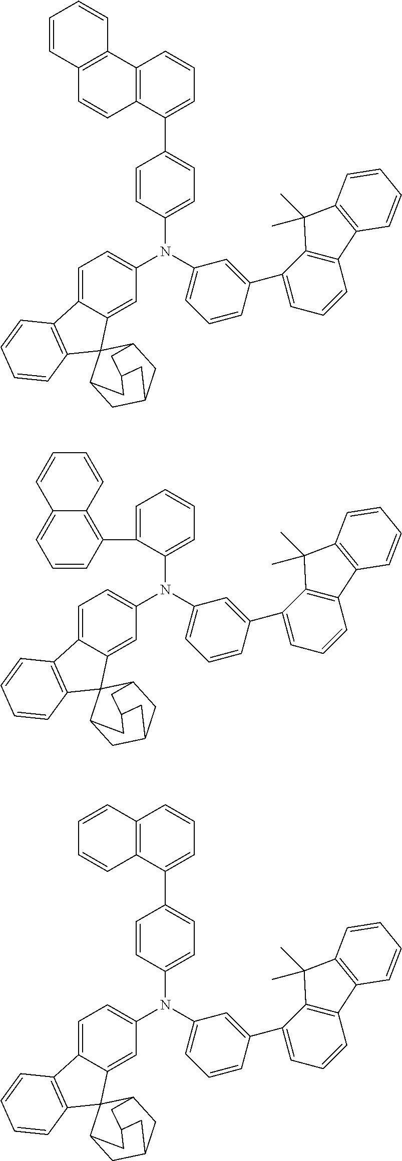 [SMILES: CC1(C)c2ccccc2-c2cccc(-c3cccc(N(c4ccc(-c5cccc6c5ccc5ccccc56)cc4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4ccccc4-c4cccc5ccccc45)c3)c21 ; CC1(C)c2ccccc2-c2cccc(-c3cccc(N(c4ccc(-c5cccc6ccccc56)cc4)c4ccc5c(c4)C4(c6ccccc6-5)C5CC6CC(C5)CC4C6)c3)c21]

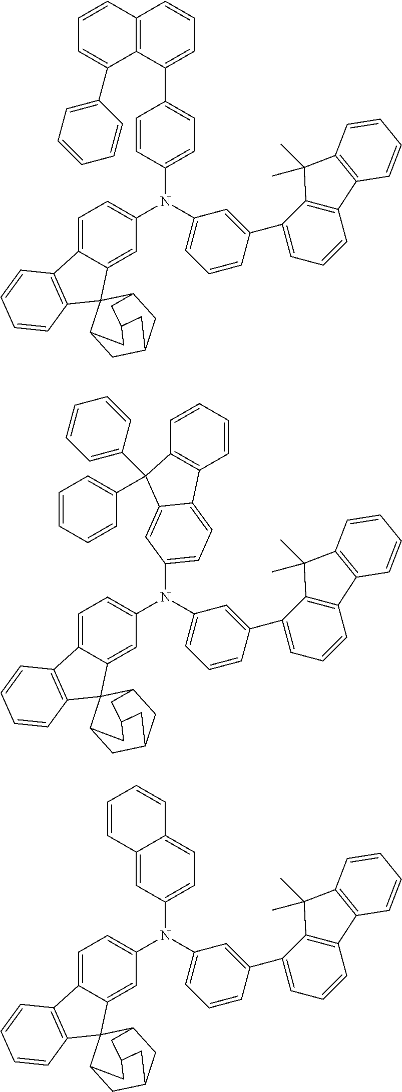

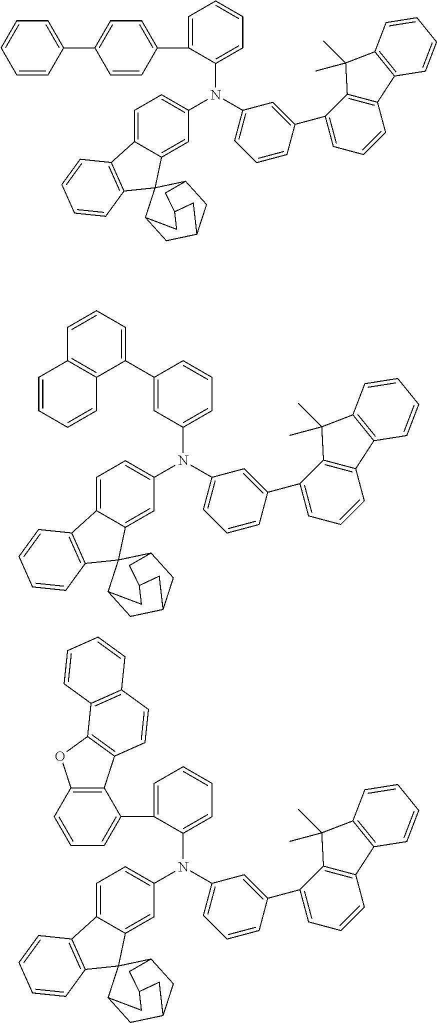

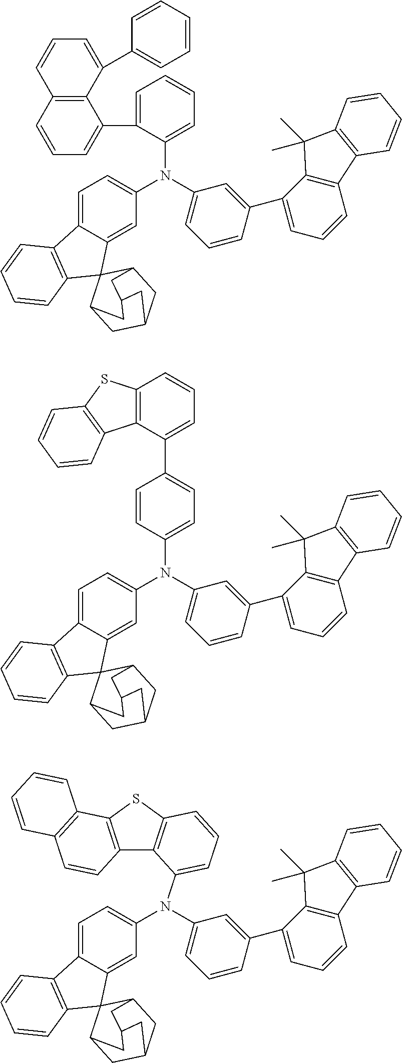 [SMILES: CC1(C)c2ccccc2-c2cccc(-c3cccc(N(c4ccc5c(c4)C4(c6ccccc6-5)C5CC6CC(C5)CC4C6)c4ccccc4-c4cccc5cccc(-c6ccccc6)c45)c3)c21 ; CC1(C)c2ccccc2-c2cccc(-c3cccc(N(c4ccc(-c5cccc6sc7ccccc7c56)cc4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4cccc5sc6c7ccccc7ccc6c45)c3)c21]

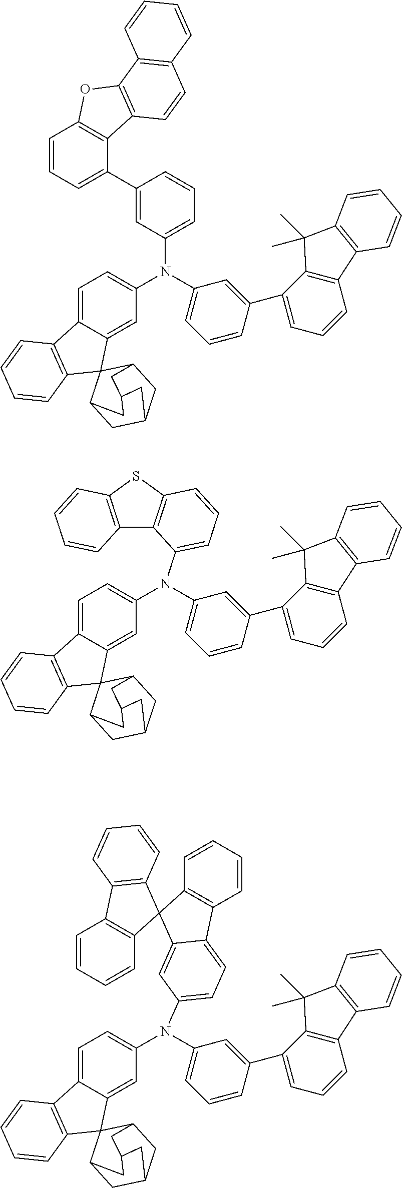 [SMILES: CC1(C)c2ccccc2-c2cccc(-c3cccc(N(c4cccc(-c5cccc6oc7c8ccccc8ccc7c56)c4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4cccc5sc6ccccc6c45)c3)c21 ; CC1(C)c2ccccc2-c2cccc(-c3cccc(N(c4ccc5c(c4)C4(c6ccccc6-c6ccccc64)c4ccccc4-5)c4ccc5c(c4)C4(c6ccccc6-5)C5CC6CC(C5)CC4C6)c3)c21]

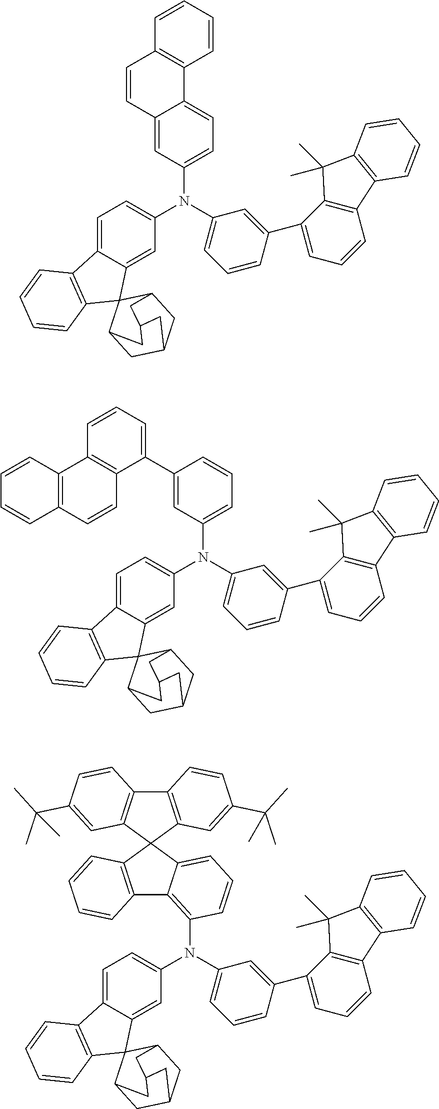 [SMILES: CC1(C)c2ccccc2-c2cccc(-c3cccc(N(c4ccc5c(c4)C4(c6ccccc6-5)C5CC6CC(C5)CC4C6)c4ccc5c(ccc6ccccc65)c4)c3)c21 ; CC1(C)c2ccccc2-c2cccc(-c3cccc(N(c4cccc(-c5cccc6c5ccc5ccccc56)c4)c4ccc5c(c4)C4(c6ccccc6-5)C5CC6CC(C5)CC4C6)c3)c21 ; CC(C)(C)c1ccc2c(c1)C1(c3cc(C(C)(C)C)ccc3-2)c2ccccc2-c2c(N(c3cccc(-c4cccc5c4C(C)(C)c4ccccc4-5)c3)c3ccc4c(c3)C3(c5ccccc5-4)C4CC5CC(C4)CC3C5)cccc21]

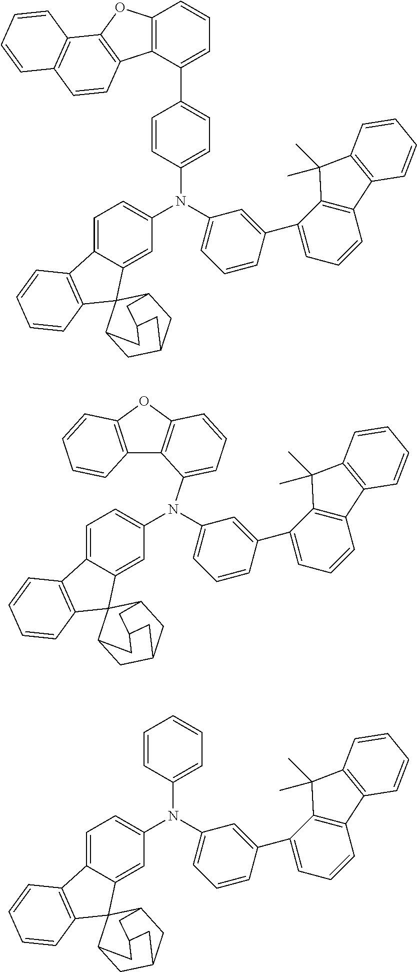 [SMILES: CC1(C)c2ccccc2-c2cccc(-c3cccc(N(c4ccc(-c5cccc6oc7c8ccccc8ccc7c56)cc4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4cccc5oc6ccccc6c45)c3)c21 ; CC1(C)c2ccccc2-c2cccc(-c3cccc(N(c4ccccc4)c4ccc5c(c4)C4(c6ccccc6-5)C5CC6CC(C5)CC4C6)c3)c21]

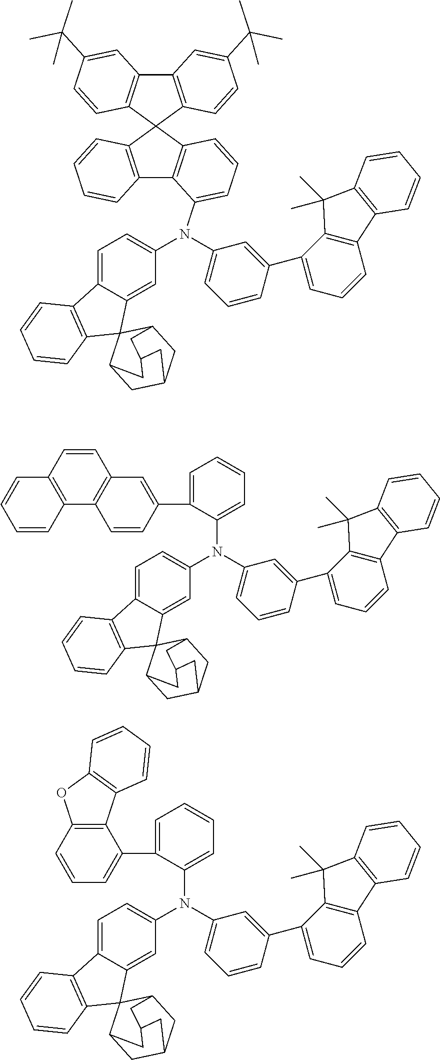 [SMILES: CC(C)(C)c1ccc2c(c1)-c1cc(C(C)(C)C)ccc1C21c2ccccc2-c2c(N(c3cccc(-c4cccc5c4C(C)(C)c4ccccc4-5)c3)c3ccc4c(c3)C3(c5ccccc5-4)C4CC5CC(C4)CC3C5)cccc21 ; CC1(C)c2ccccc2-c2cccc(-c3cccc(N(c4ccc5c(c4)C4(c6ccccc6-5)C5CC6CC(C5)CC4C6)c4ccccc4-c4ccc5c(ccc6ccccc65)c4)c3)c21 ; CC1(C)c2ccccc2-c2cccc(-c3cccc(N(c4ccc5c(c4)C4(c6ccccc6-5)C5CC6CC(C5)CC4C6)c4ccccc4-c4cccc5oc6ccccc6c45)c3)c21]

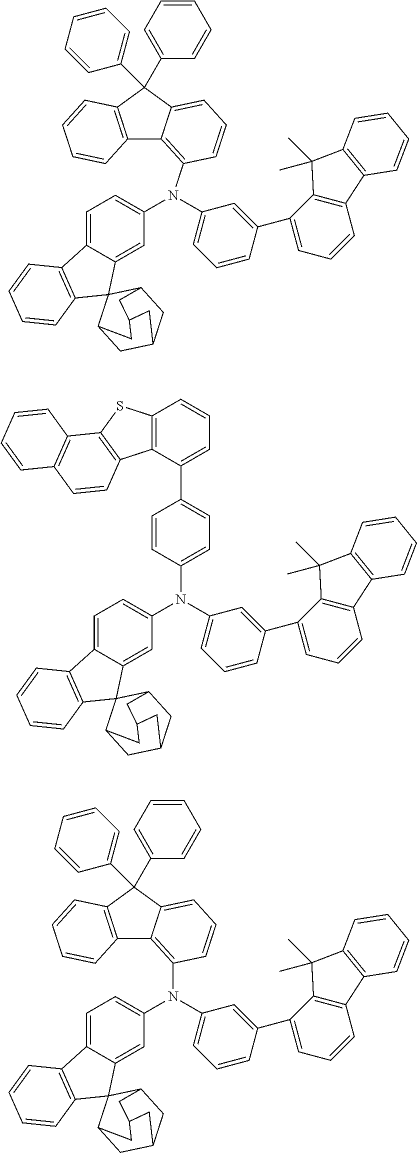 [SMILES: CC1(C)c2ccccc2-c2cccc(-c3cccc(N(c4ccc5c(c4)C4(c6ccccc6-5)C5CC6CC(C5)CC4C6)c4cccc5c4-c4ccccc4C5(c4ccccc4)c4ccccc4)c3)c21 ; CC1(C)c2ccccc2-c2cccc(-c3cccc(N(c4ccc(-c5cccc6sc7c8ccccc8ccc7c56)cc4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4cccc5c4-c4ccccc4C5(c4ccccc4)c4ccccc4)c3)c21]

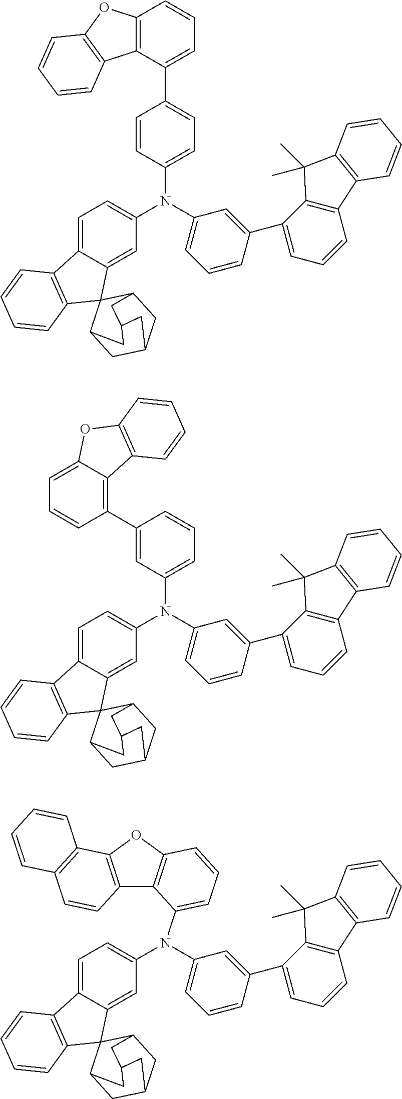 [SMILES: CC1(C)c2ccccc2-c2cccc(-c3cccc(N(c4ccc(-c5cccc6oc7ccccc7c56)cc4)c4ccc5c(c4)C4(c6ccccc6-5)C5CC6CC(C5)CC4C6)c3)c21 ; CC1(C)c2ccccc2-c2cccc(-c3cccc(N(c4cccc(-c5cccc6oc7ccccc7c56)c4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4cccc5oc6c7ccccc7ccc6c45)c3)c21]

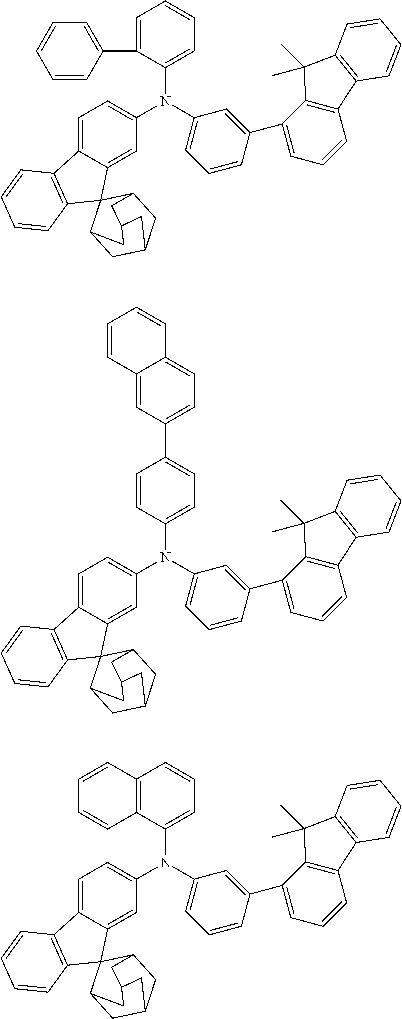 [SMILES: CC1(C)c2ccccc2-c2cccc(-c3cccc(N(c4ccc5c(c4)C4(c6ccccc6-5)C5CC6CC(C5)CC4C6)c4ccccc4-c4ccccc4)c3)c21 ; CC1(C)c2ccccc2-c2cccc(-c3cccc(N(c4ccc(-c5ccc6ccccc6c5)cc4)c4ccc5c(c4)C4(c6ccccc6-5)C5CC6CC(C5)CC4C6)c3)c21 ; CC1(C)c2ccccc2-c2cccc(-c3cccc(N(c4ccc5c(c4)C4(c6ccccc6-5)C5CC6CC(C5)CC4C6)c4cccc5ccccc45)c3)c21]

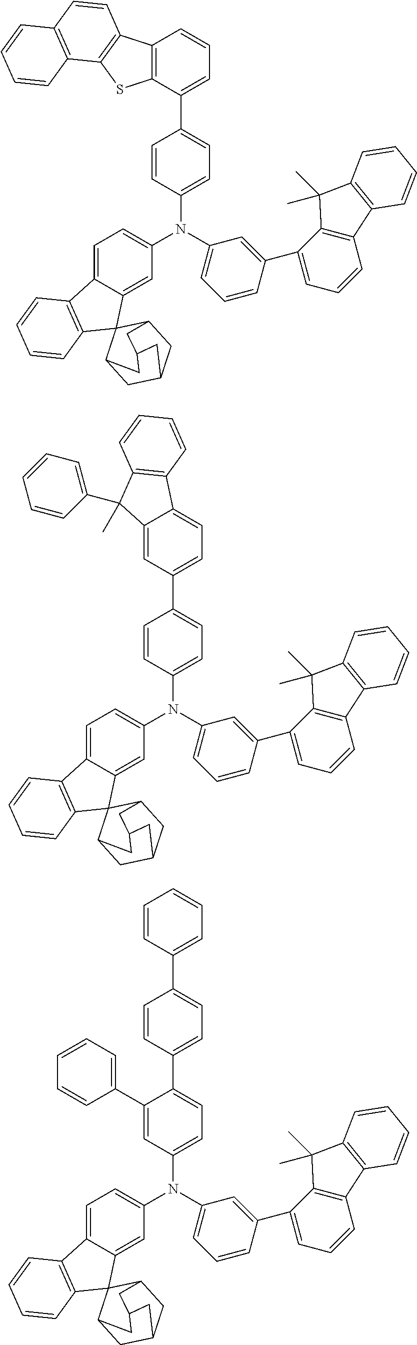

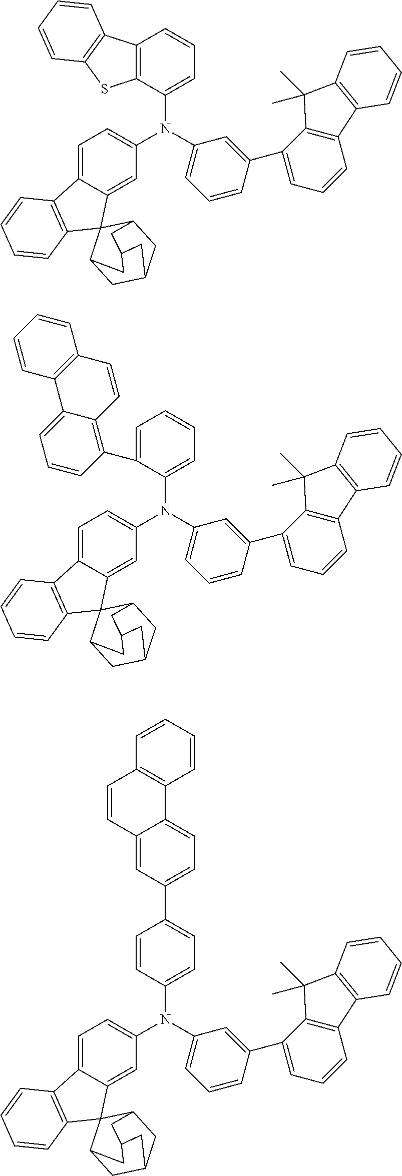 [SMILES: CC1(C)c2ccccc2-c2cccc(-c3cccc(N(c4ccc5c(c4)C4(c6ccccc6-5)C5CC6CC(C5)CC4C6)c4cccc5c4sc4ccccc45)c3)c21 ; CC1(C)c2ccccc2-c2cccc(-c3cccc(N(c4ccc5c(c4)C4(c6ccccc6-5)C5CC6CC(C5)CC4C6)c4ccccc4-c4cccc5c4ccc4ccccc45)c3)c21 ; CC1(C)c2ccccc2-c2cccc(-c3cccc(N(c4ccc(-c5ccc6c(ccc7ccccc76)c5)cc4)c4ccc5c(c4)C4(c6ccccc6-5)C5CC6CC(C5)CC4C6)c3)c21]

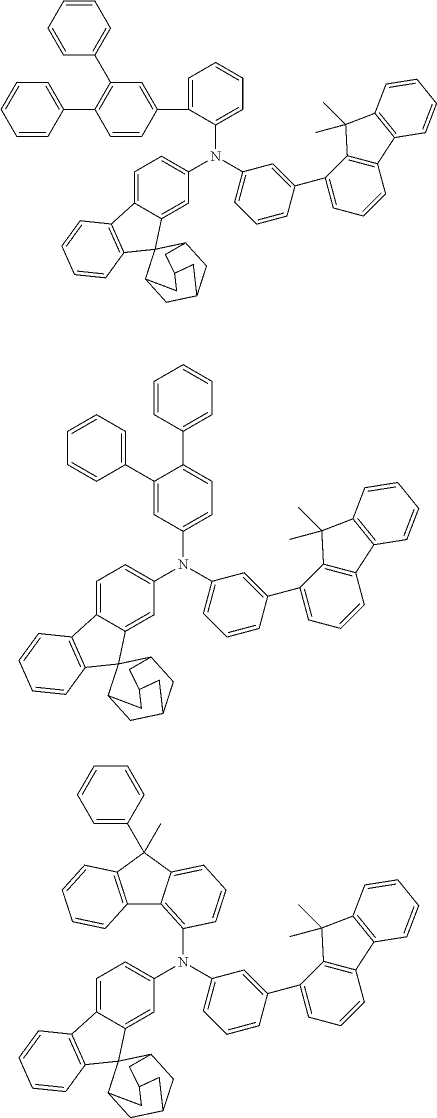

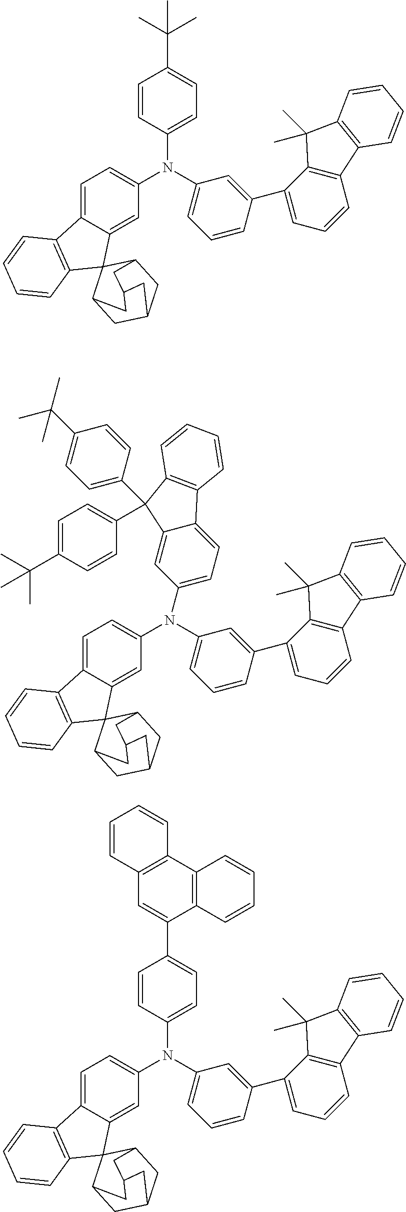

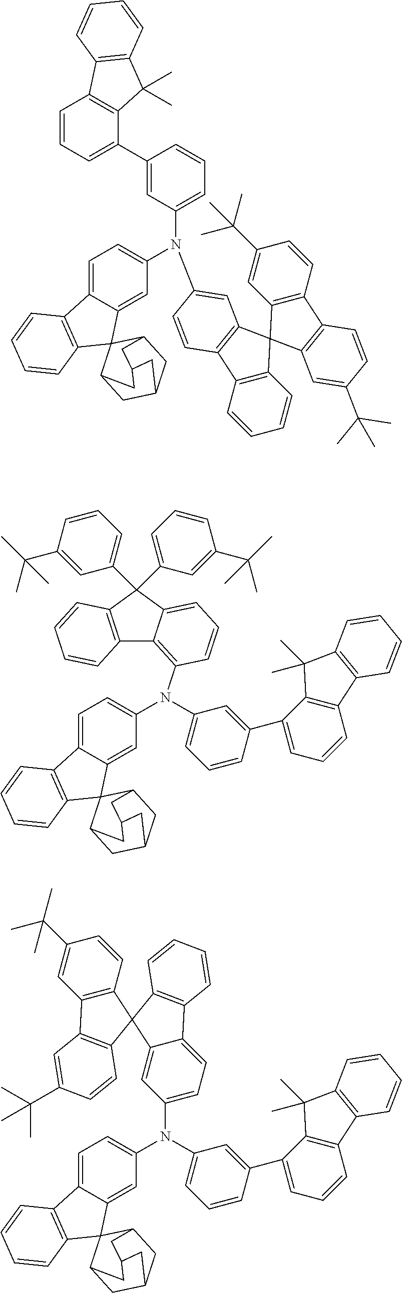 [SMILES: CC(C)(C)c1ccc2c(c1)C1(c3ccccc3-c3ccc(N(c4cccc(-c5cccc6c5C(C)(C)c5ccccc5-6)c4)c4ccc5c(c4)C4(c6ccccc6-5)C5CC6CC(C5)CC4C6)cc31)c1cc(C(C)(C)C)ccc1-2 ; CC(C)(C)c1cccc(C2(c3cccc(C(C)(C)C)c3)c3ccccc3-c3c(N(c4cccc(-c5cccc6c5C(C)(C)c5ccccc5-6)c4)c4ccc5c(c4)C4(c6ccccc6-5)C5CC6CC(C5)CC4C6)cccc32)c1 ; CC(C)(C)c1ccc2c(c1)-c1cc(C(C)(C)C)ccc1C21c2ccccc2-c2ccc(N(c3cccc(-c4cccc5c4C(C)(C)c4ccccc4-5)c3)c3ccc4c(c3)C3(c5ccccc5-4)C4CC5CC(C4)CC3C5)cc21]

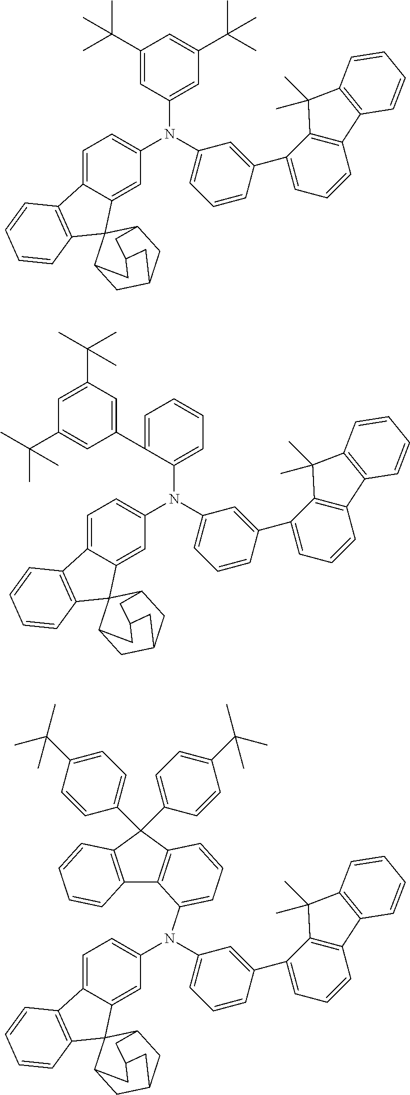

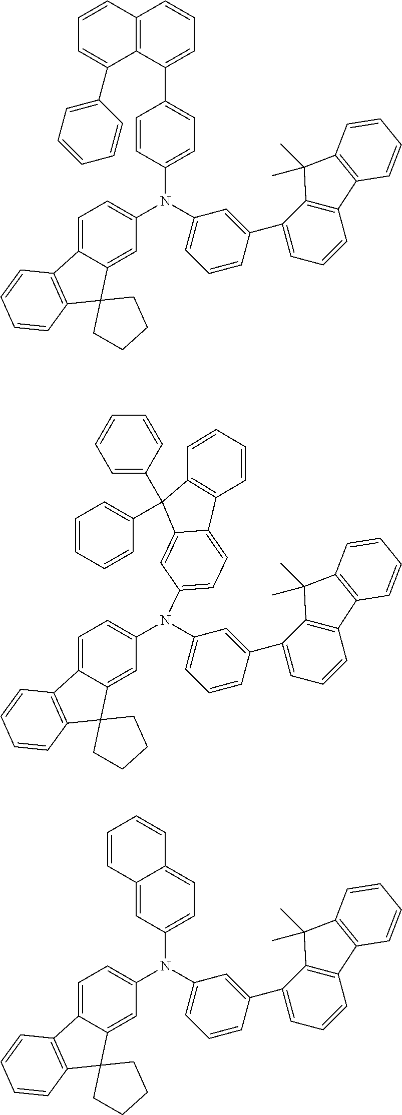 [SMILES: CC1(C)c2ccccc2-c2cccc(-c3cccc(N(c4ccc(-c5cccc6cccc(-c7ccccc7)c56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4ccc5c(c4)C(c4ccccc4)(c4ccccc4)c4ccccc4-5)c3)c21 ; CC1(C)c2ccccc2-c2cccc(-c3cccc(N(c4ccc5c(c4)C4(CCCC4)c4ccccc4-5)c4ccc5ccccc5c4)c3)c21]

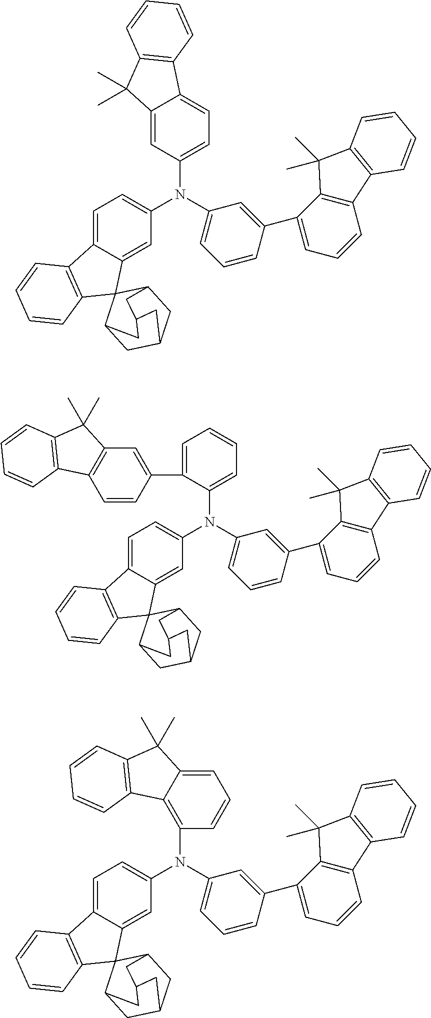 [SMILES: CC1(C)c2ccccc2-c2ccc(N(c3cccc(-c4cccc5c4C(C)(C)c4ccccc4-5)c3)c3ccc4c(c3)C3(c5ccccc5-4)C4CC5CC(C4)CC3C5)cc21 ; CC1(C)c2ccccc2-c2ccc(-c3ccccc3N(c3cccc(-c4cccc5c4C(C)(C)c4ccccc4-5)c3)c3ccc4c(c3)C3(c5ccccc5-4)C4CC5CC(C4)CC3C5)cc21 ; CC1(C)c2ccccc2-c2c(N(c3cccc(-c4cccc5c4C(C)(C)c4ccccc4-5)c3)c3ccc4c(c3)C3(c5ccccc5-4)C4CC5CC(C4)CC3C5)cccc21]

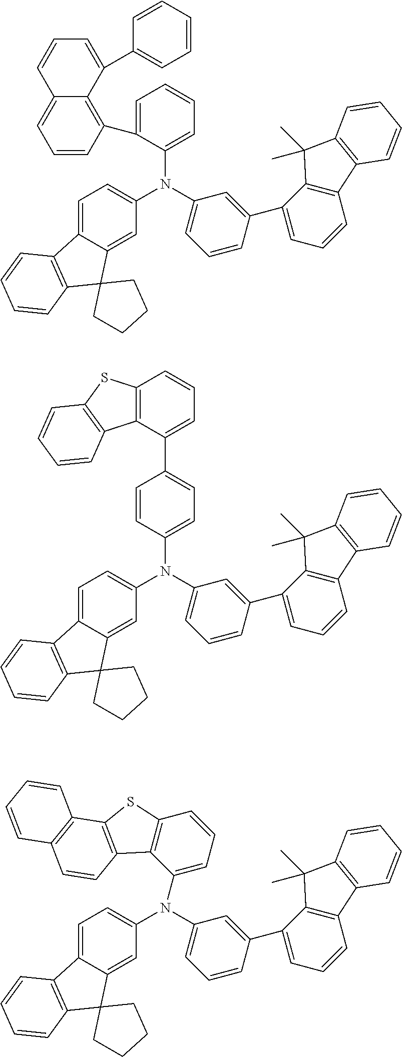 [SMILES: CC1(C)c2ccccc2-c2cccc(-c3cccc(N(c4ccc5c(c4)C4(CCCC4)c4ccccc4-5)c4ccccc4-c4cccc5cccc(-c6ccccc6)c45)c3)c21 ; CC1(C)c2ccccc2-c2cccc(-c3cccc(N(c4ccc(-c5cccc6sc7ccccc7c56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5sc6c7ccccc7ccc6c45)c3)c21]

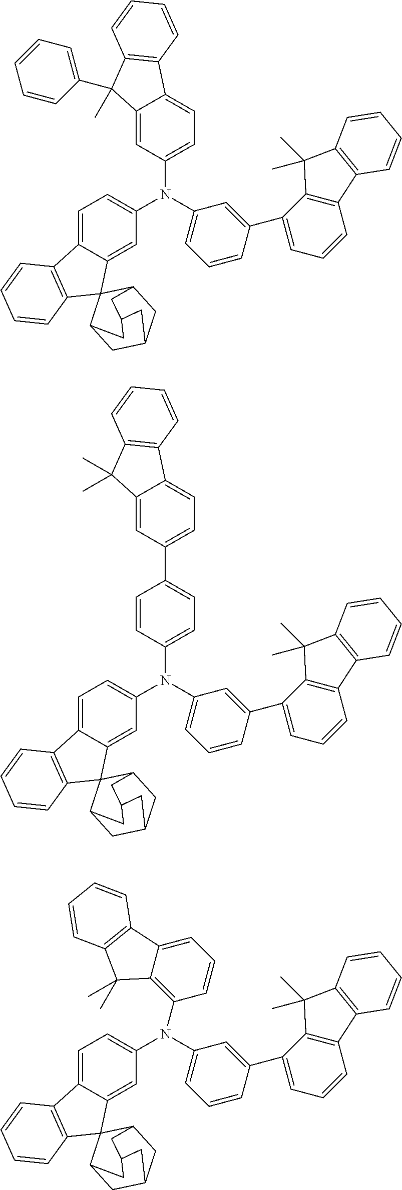

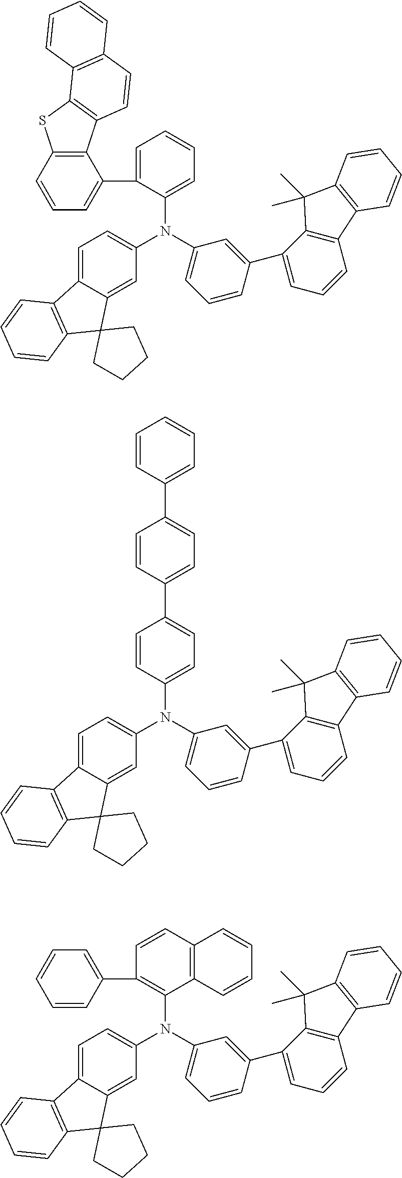 [SMILES: CC1(C)c2ccccc2-c2cccc(-c3cccc(N(c4ccc5c(c4)C4(CCCC4)c4ccccc4-5)c4ccccc4-c4cccc5sc6c7ccccc7ccc6c45)c3)c21 ; CC1(C)c2ccccc2-c2cccc(-c3cccc(N(c4ccc(-c5ccc(-c6ccccc6)cc5)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4c(-c5ccccc5)ccc5ccccc45)c3)c21]

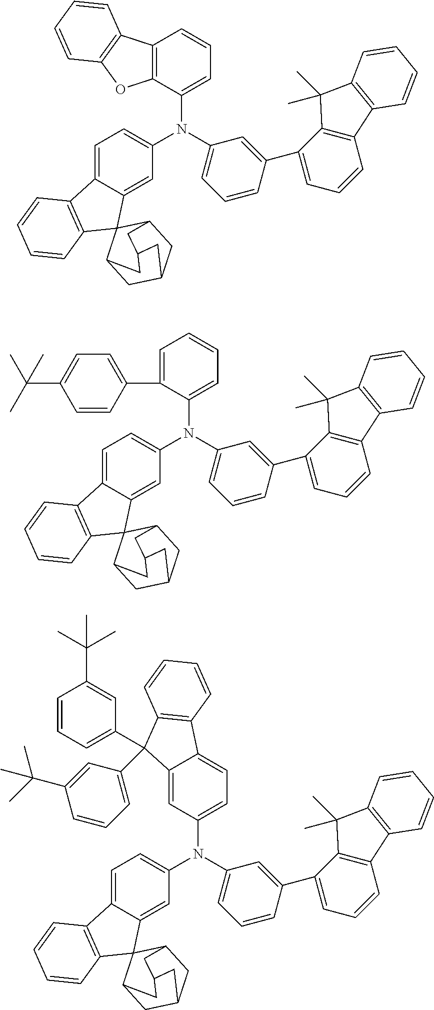 [SMILES: CC1(C)c2ccccc2-c2cccc(-c3cccc(N(c4ccc5c(c4)C4(c6ccccc6-5)C5CC6CC(C5)CC4C6)c4cccc5c4oc4ccccc45)c3)c21 ; CC(C)(C)c1ccc(-c2ccccc2N(c2cccc(-c3cccc4c3C(C)(C)c3ccccc3-4)c2)c2ccc3c(c2)C2(c4ccccc4-3)C3CC4CC(C3)CC2C4)cc1 ; CC(C)(C)c1cccc(C2(c3cccc(C(C)(C)C)c3)c3ccccc3-c3ccc(N(c4cccc(-c5cccc6c5C(C)(C)c5ccccc5-6)c4)c4ccc5c(c4)C4(c6ccccc6-5)C5CC6CC(C5)CC4C6)cc32)c1]

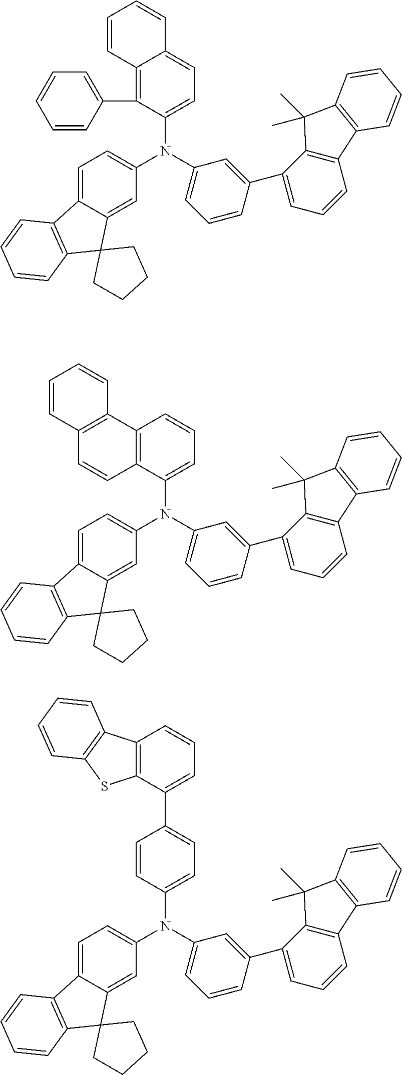 [SMILES: CC1(C)c2ccccc2-c2cccc(-c3cccc(N(c4ccc5c(c4)C4(CCCC4)c4ccccc4-5)c4ccc5ccccc5c4-c4ccccc4)c3)c21 ; CC1(C)c2ccccc2-c2cccc(-c3cccc(N(c4ccc5c(c4)C4(CCCC4)c4ccccc4-5)c4cccc5c4ccc4ccccc45)c3)c21 ; CC1(C)c2ccccc2-c2cccc(-c3cccc(N(c4ccc(-c5cccc6c5sc5ccccc56)cc4)c4ccc5c(c4)C4(CCCC4)c4ccccc4-5)c3)c21]

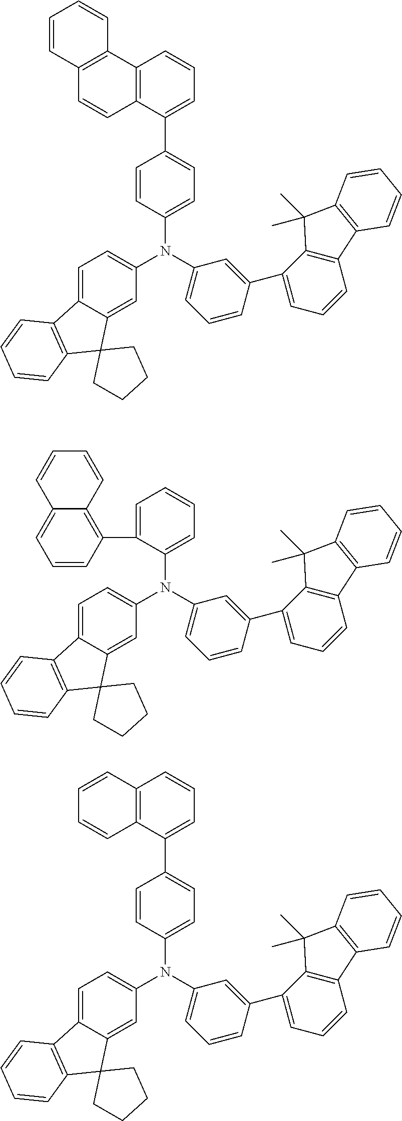

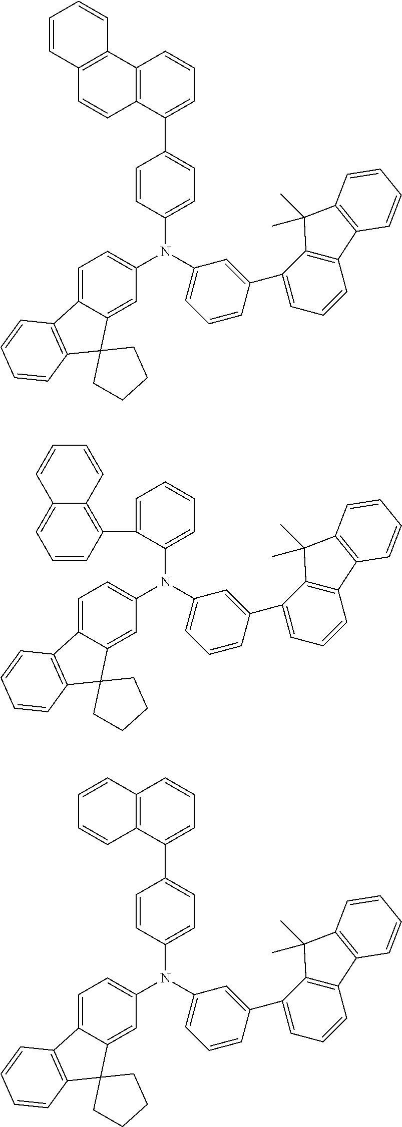 [SMILES: CC1(C)c2ccccc2-c2cccc(-c3cccc(N(c4ccc(-c5cccc6c5ccc5ccccc56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4ccccc4-c4cccc5ccccc45)c3)c21 ; CC1(C)c2ccccc2-c2cccc(-c3cccc(N(c4ccc(-c5cccc6ccccc56)cc4)c4ccc5c(c4)C4(CCCC4)c4ccccc4-5)c3)c21]

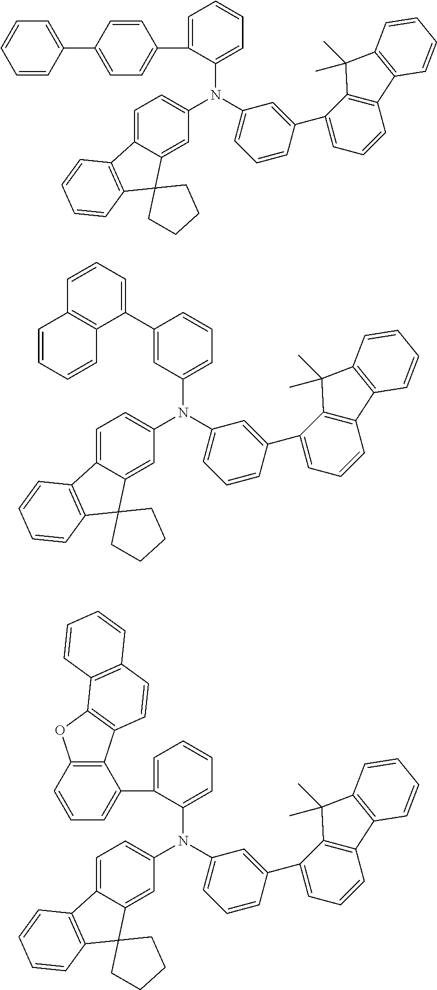 [SMILES: CC1(C)c2ccccc2-c2cccc(-c3cccc(N(c4ccc5c(c4)C4(CCCC4)c4ccccc4-5)c4ccccc4-c4ccc(-c5ccccc5)cc4)c3)c21 ; CC1(C)c2ccccc2-c2cccc(-c3cccc(N(c4cccc(-c5cccc6ccccc56)c4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4ccccc4-c4cccc5oc6c7ccccc7ccc6c45)c3)c21]

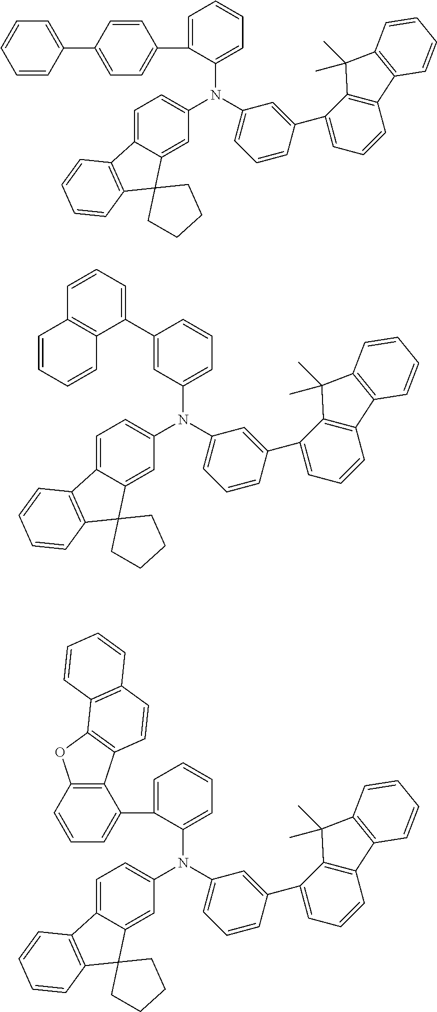 [SMILES: CC1(C)c2ccccc2-c2cccc(-c3cccc(N(c4ccc5c(c4)C4(CCCC4)c4ccccc4-5)c4ccccc4-c4ccc(-c5ccccc5)cc4)c3)c21 ; CC1(C)c2ccccc2-c2cccc(-c3cccc(N(c4cccc(-c5cccc6ccccc56)c4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4ccccc4-c4cccc5oc6c7ccccc7ccc6c45)c3)c21]

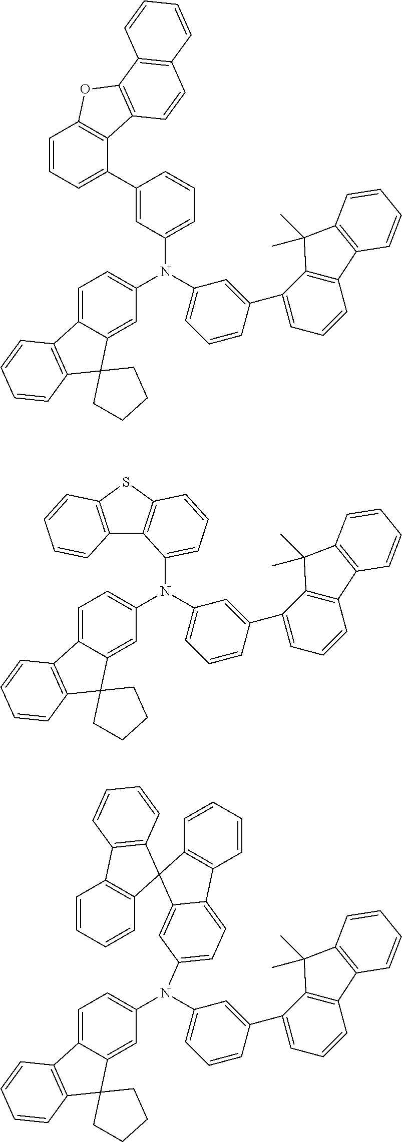 [SMILES: CC1(C)c2ccccc2-c2cccc(-c3cccc(N(c4cccc(-c5cccc6oc7c8ccccc8ccc7c56)c4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5sc6ccccc6c45)c3)c21 ; CC1(C)c2ccccc2-c2cccc(-c3cccc(N(c4ccc5c(c4)C4(CCCC4)c4ccccc4-5)c4ccc5c(c4)C4(c6ccccc6-c6ccccc64)c4ccccc4-5)c3)c21]

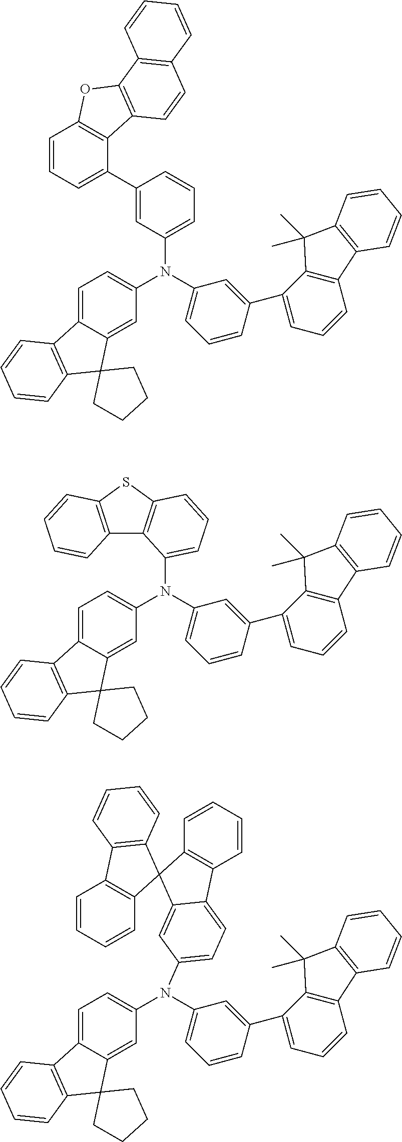 [SMILES: CC1(C)c2ccccc2-c2cccc(-c3cccc(N(c4cccc(-c5cccc6oc7c8ccccc8ccc7c56)c4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5sc6ccccc6c45)c3)c21 ; CC1(C)c2ccccc2-c2cccc(-c3cccc(N(c4ccc5c(c4)C4(CCCC4)c4ccccc4-5)c4ccc5c(c4)C4(c6ccccc6-c6ccccc64)c4ccccc4-5)c3)c21]

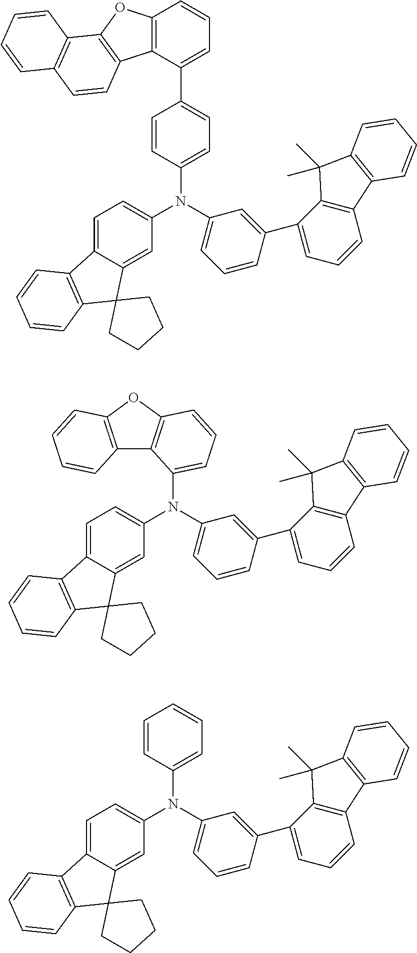 [SMILES: CC1(C)c2ccccc2-c2cccc(-c3cccc(N(c4ccc(-c5cccc6oc7c8ccccc8ccc7c56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5oc6ccccc6c45)c3)c21 ; CC1(C)c2ccccc2-c2cccc(-c3cccc(N(c4ccccc4)c4ccc5c(c4)C4(CCCC4)c4ccccc4-5)c3)c21]

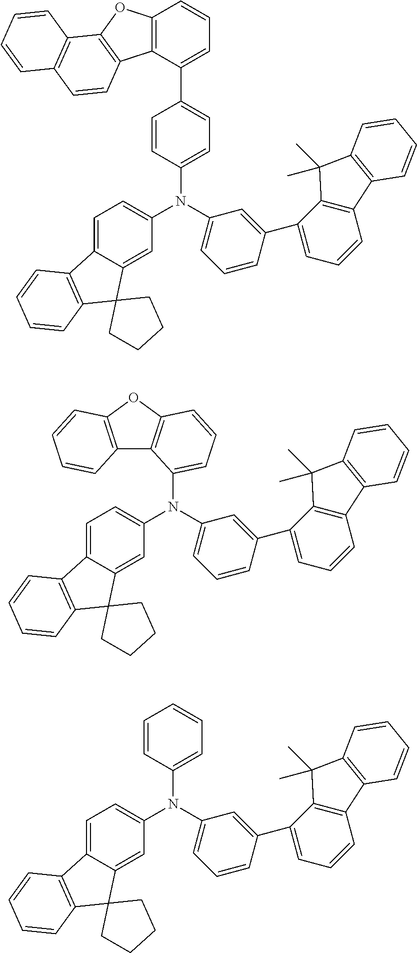 [SMILES: CC1(C)c2ccccc2-c2cccc(-c3cccc(N(c4ccc(-c5cccc6oc7c8ccccc8ccc7c56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5oc6ccccc6c45)c3)c21 ; CC1(C)c2ccccc2-c2cccc(-c3cccc(N(c4ccccc4)c4ccc5c(c4)C4(CCCC4)c4ccccc4-5)c3)c21]

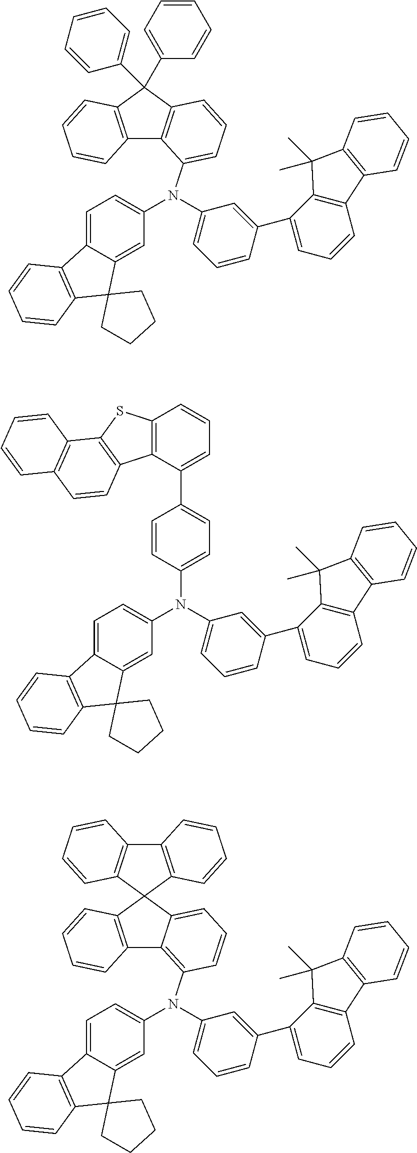 [SMILES: CC1(C)c2ccccc2-c2cccc(-c3cccc(N(c4ccc5c(c4)C4(CCCC4)c4ccccc4-5)c4cccc5c4-c4ccccc4C5(c4ccccc4)c4ccccc4)c3)c21 ; CC1(C)c2ccccc2-c2cccc(-c3cccc(N(c4ccc(-c5cccc6sc7c8ccccc8ccc7c56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5c4-c4ccccc4C54c5ccccc5-c5ccccc54)c3)c21]

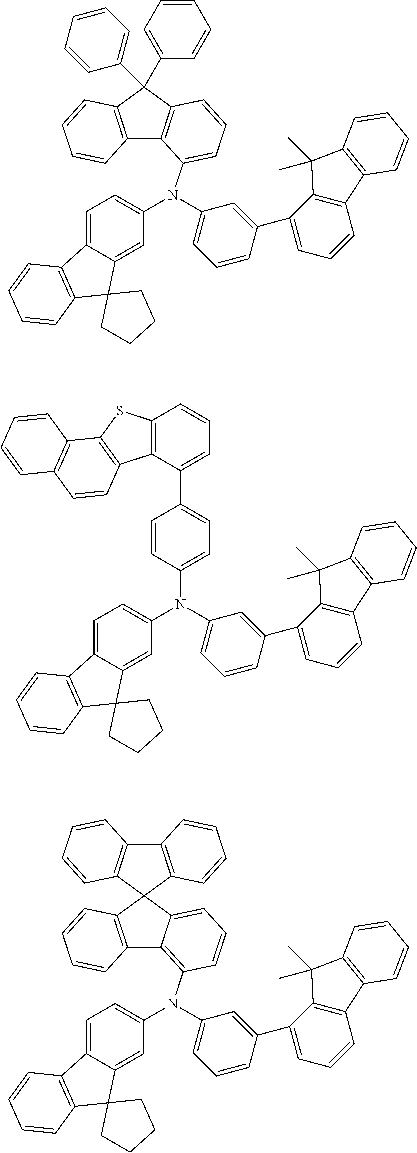 [SMILES: CC1(C)c2ccccc2-c2cccc(-c3cccc(N(c4ccc5c(c4)C4(CCCC4)c4ccccc4-5)c4cccc5c4-c4ccccc4C5(c4ccccc4)c4ccccc4)c3)c21 ; CC1(C)c2ccccc2-c2cccc(-c3cccc(N(c4ccc(-c5cccc6sc7c8ccccc8ccc7c56)cc4)c4ccc5c(c4)C4(CCCC4)c4ccccc4-5)c3)c21 ; CC1(C)c2ccccc2-c2cccc(-c3cccc(N(c4ccc5c(c4)C4(CCCC4)c4ccccc4-5)c4cccc5c4-c4ccccc4C54c5ccccc5-c5ccccc54)c3)c21]

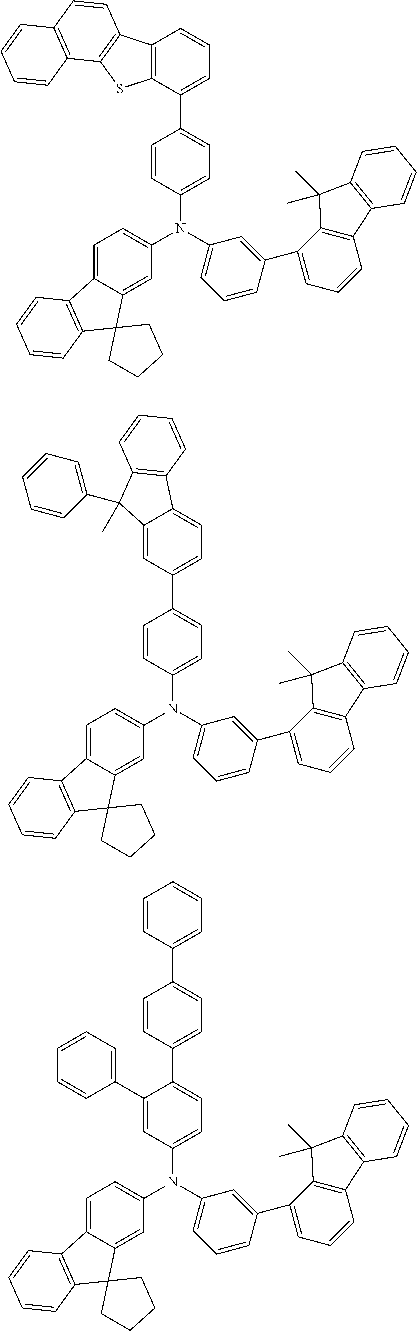

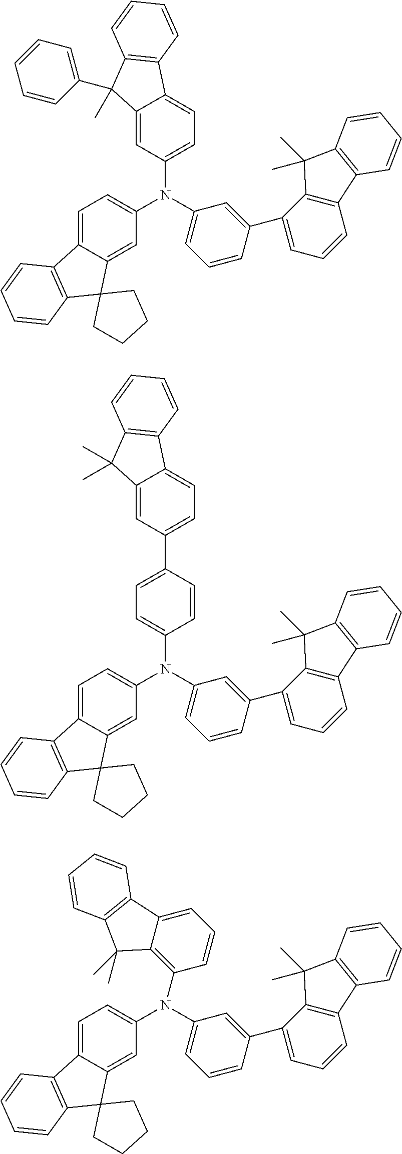

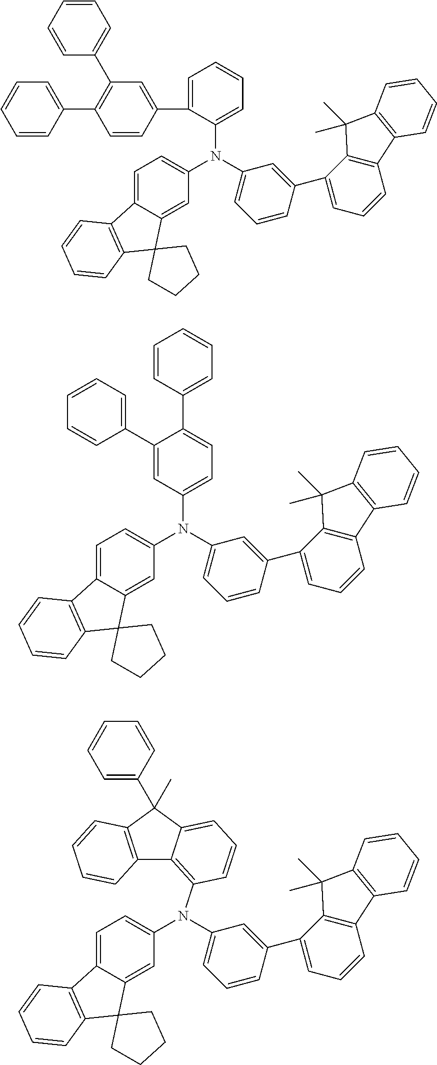

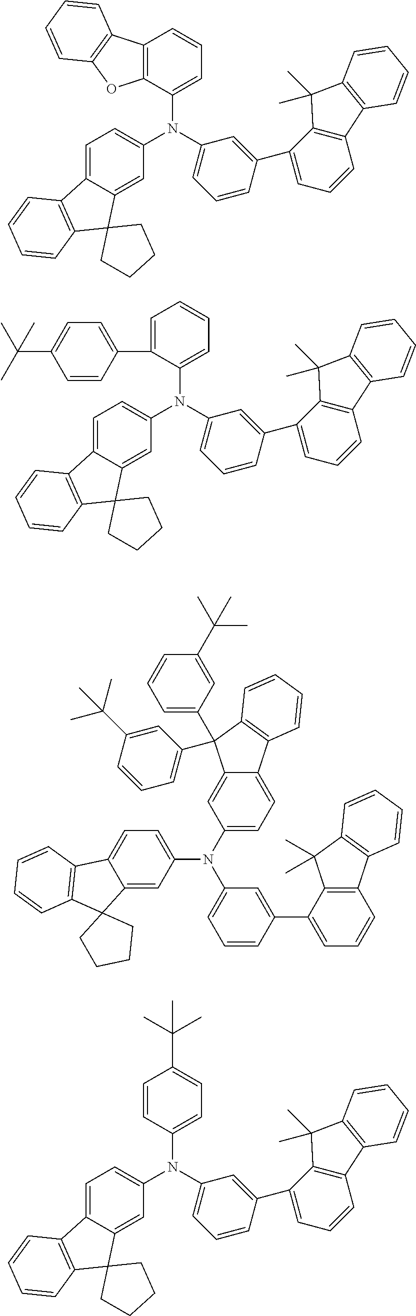

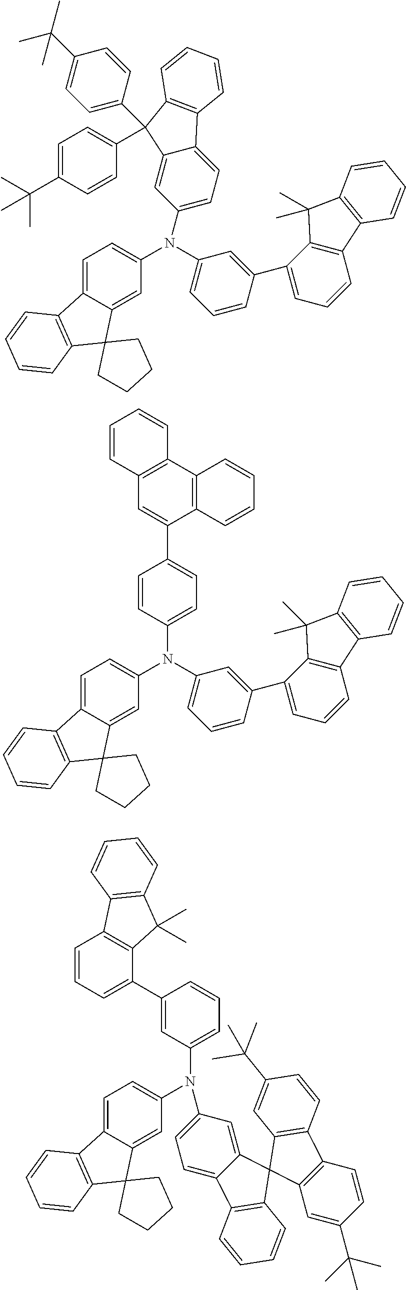

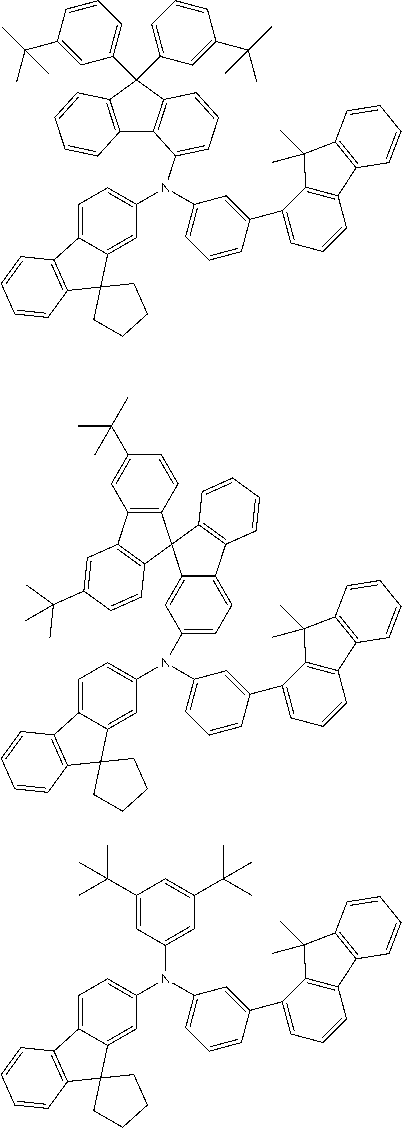

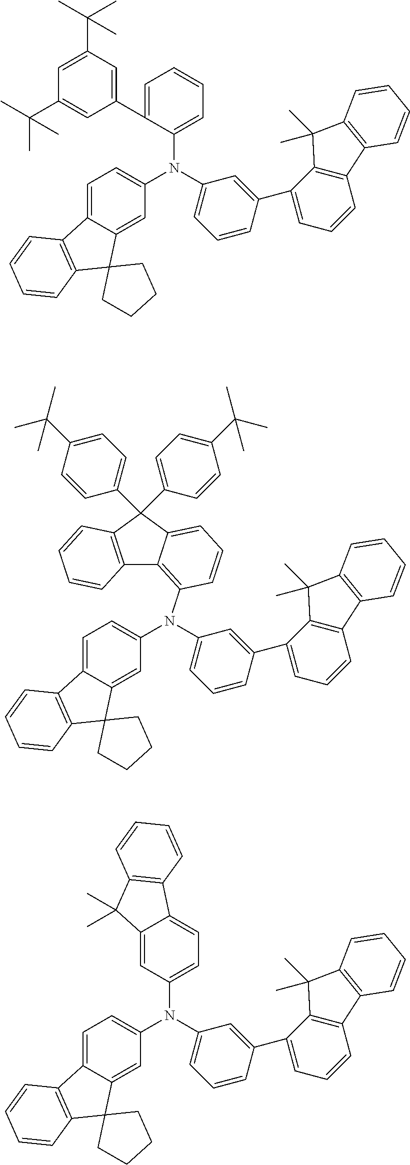 [SMILES: CC(C)(C)c1cc(-c2ccccc2N(c2cccc(-c3cccc4c3C(C)(C)c3ccccc3-4)c2)c2ccc3c(c2)C2(CCCC2)c2ccccc2-3)cc(C(C)(C)C)c1 ; CC(C)(C)c1ccc(C2(c3ccc(C(C)(C)C)cc3)c3ccccc3-c3c(N(c4cccc(-c5cccc6c5C(C)(C)c5ccccc5-6)c4)c4ccc5c(c4)C4(CCCC4)c4ccccc4-5)cccc32)cc1 ; CC1(C)c2ccccc2-c2ccc(N(c3cccc(-c4cccc5c4C(C)(C)c4ccccc4-5)c3)c3ccc4c(c3)C3(CCCC3)c3ccccc3-4)cc21]

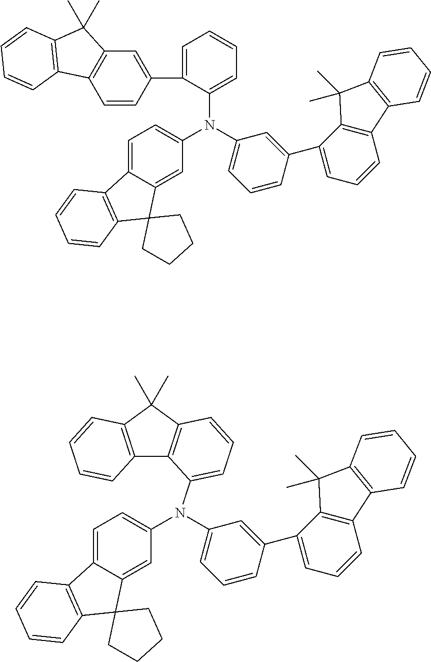 [SMILES: CC1(C)c2ccccc2-c2ccc(-c3ccccc3N(c3cccc(-c4cccc5c4C(C)(C)c4ccccc4-5)c3)c3ccc4c(c3)C3(CCCC3)c3ccccc3-4)cc21 ; CC1(C)c2ccccc2-c2c(N(c3cccc(-c4cccc5c4C(C)(C)c4ccccc4-5)c3)c3ccc4c(c3)C3(CCCC3)c3ccccc3-4)cccc21]

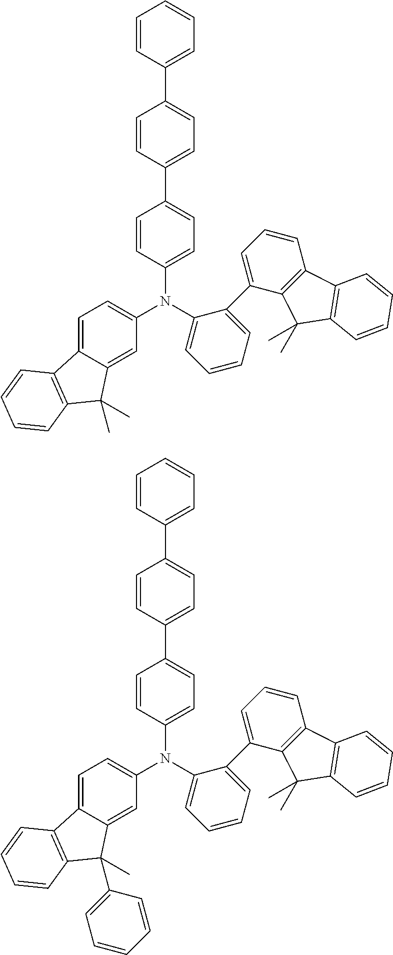 [SMILES: CC1(C)c2ccccc2-c2ccc(N(c3ccc(-c4ccc(-c5ccccc5)cc4)cc3)c3ccccc3-c3cccc4c3C(C)(C)c3ccccc3-4)cc21 ; CC1(C)c2ccccc2-c2cccc(-c3ccccc3N(c3ccc(-c4ccc(-c5ccccc5)cc4)cc3)c3ccc4c(c3)C(C)(c3ccccc3)c3ccccc3-4)c21]

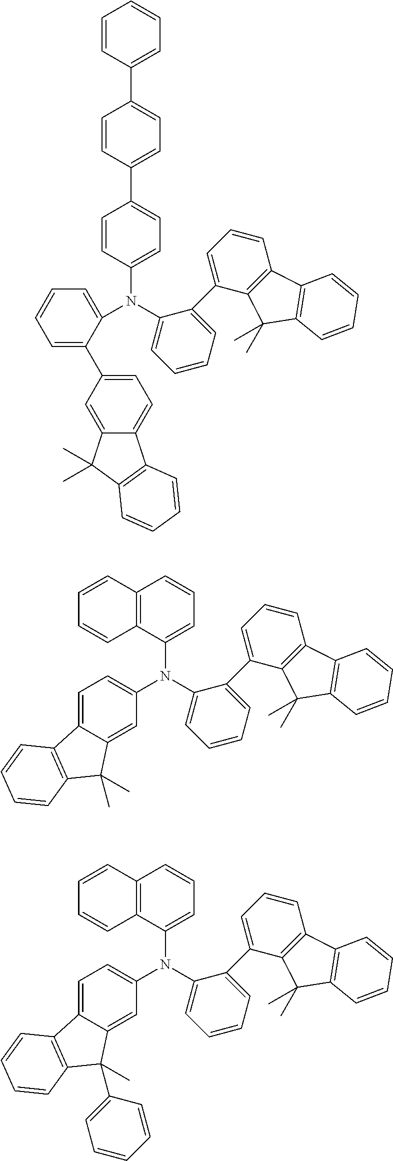

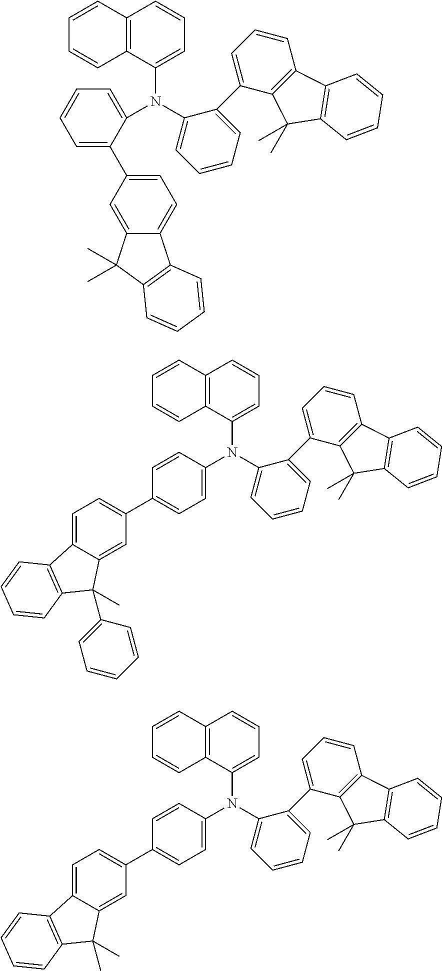

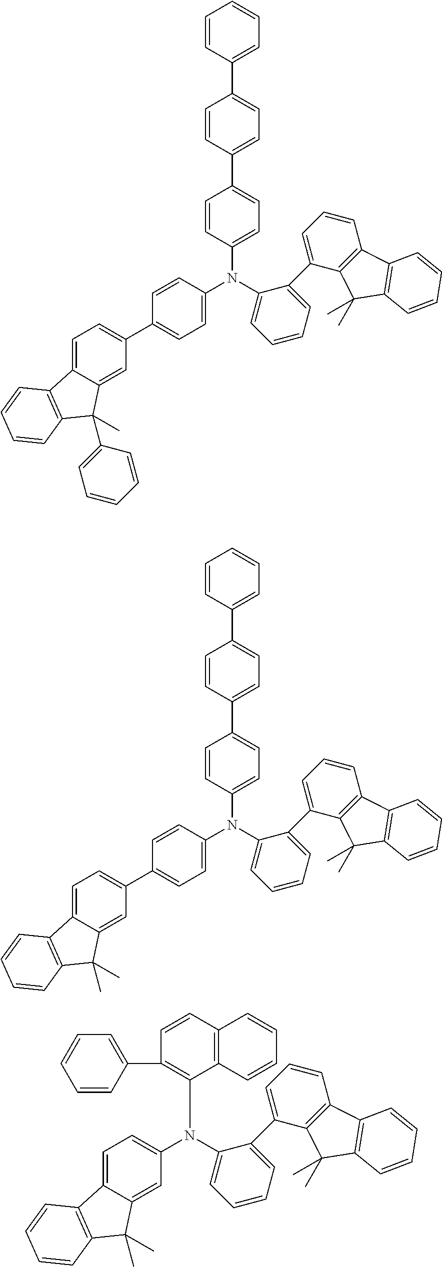 [SMILES: CC1(C)c2ccccc2-c2cccc(-c3ccccc3N(c3ccc(-c4ccc(-c5ccccc5)cc4)cc3)c3ccc(-c4ccc5c(c4)C(C)(c4ccccc4)c4ccccc4-5)cc3)c21 ; CC1(C)c2ccccc2-c2ccc(-c3ccc(N(c4ccc(-c5ccc(-c6ccccc6)cc5)cc4)c4ccccc4-c4cccc5c4C(C)(C)c4ccccc4-5)cc3)cc21 ; CC1(C)c2ccccc2-c2ccc(N(c3ccccc3-c3cccc4c3C(C)(C)c3ccccc3-4)c3c(-c4ccccc4)ccc4ccccc34)cc21]

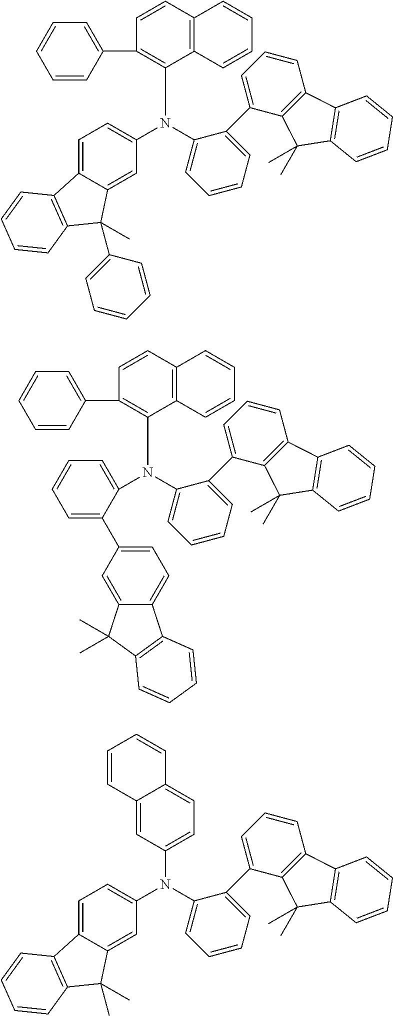

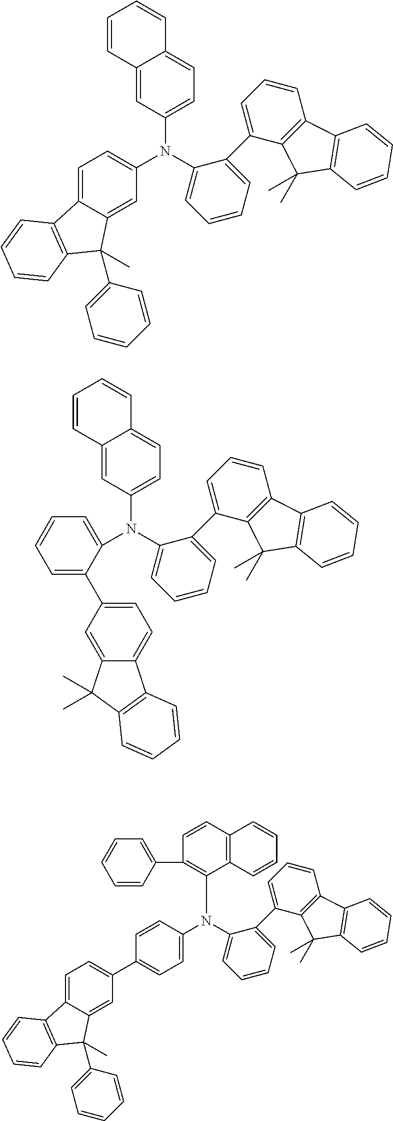

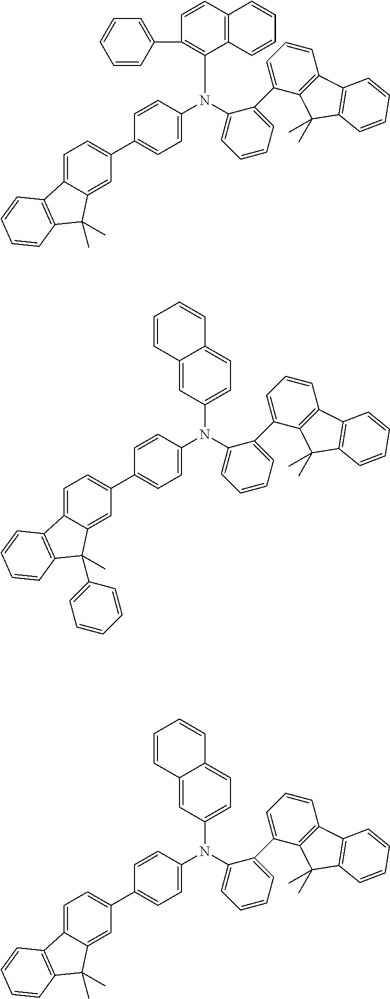 [SMILES: CC1(C)c2ccccc2-c2ccc(-c3ccc(N(c4ccccc4-c4cccc5c4C(C)(C)c4ccccc4-5)c4c(-c5ccccc5)ccc5ccccc45)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccc4ccccc4c3)c21 ; CC1(C)c2ccccc2-c2ccc(-c3ccc(N(c4ccc5ccccc5c4)c4ccccc4-c4cccc5c4C(C)(C)c4ccccc4-5)cc3)cc21]

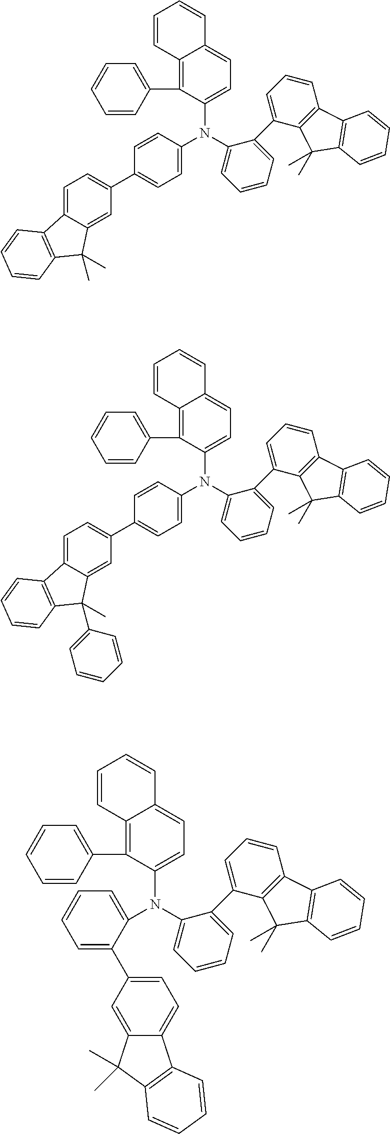 [SMILES: CC1(C)c2ccccc2-c2ccc(-c3ccc(N(c4ccccc4-c4cccc5c4C(C)(C)c4ccccc4-5)c4ccc5ccccc5c4-c4ccccc4)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccc4ccccc4c3-c3ccccc3)c21 ; CC1(C)c2ccccc2-c2ccc(-c3ccccc3N(c3ccccc3-c3cccc4c3C(C)(C)c3ccccc3-4)c3ccc4ccccc4c3-c3ccccc3)cc21]

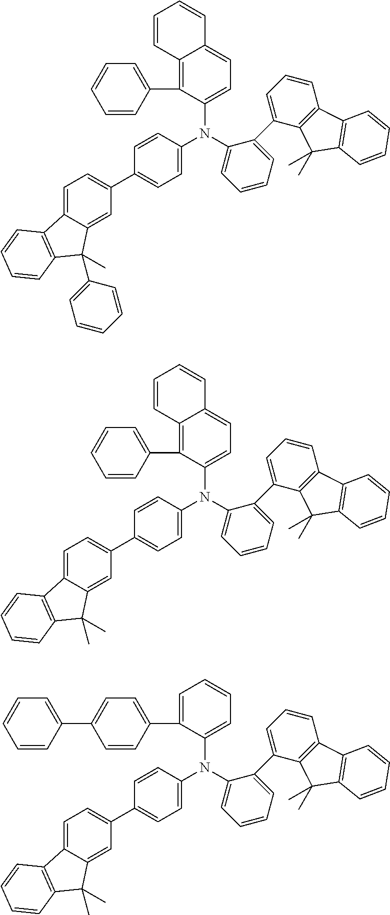 [SMILES: CC1(C)c2ccccc2-c2cccc(-c3ccccc3N(c3ccc(-c4ccc5c(c4)C(C)(c4ccccc4)c4ccccc4-5)cc3)c3ccc4ccccc4c3-c3ccccc3)c21 ; CC1(C)c2ccccc2-c2ccc(-c3ccc(N(c4ccccc4-c4cccc5c4C(C)(C)c4ccccc4-5)c4ccc5ccccc5c4-c4ccccc4)cc3)cc21 ; CC1(C)c2ccccc2-c2ccc(-c3ccc(N(c4ccccc4-c4ccc(-c5ccccc5)cc4)c4ccccc4-c4cccc5c4C(C)(C)c4ccccc4-5)cc3)cc21]

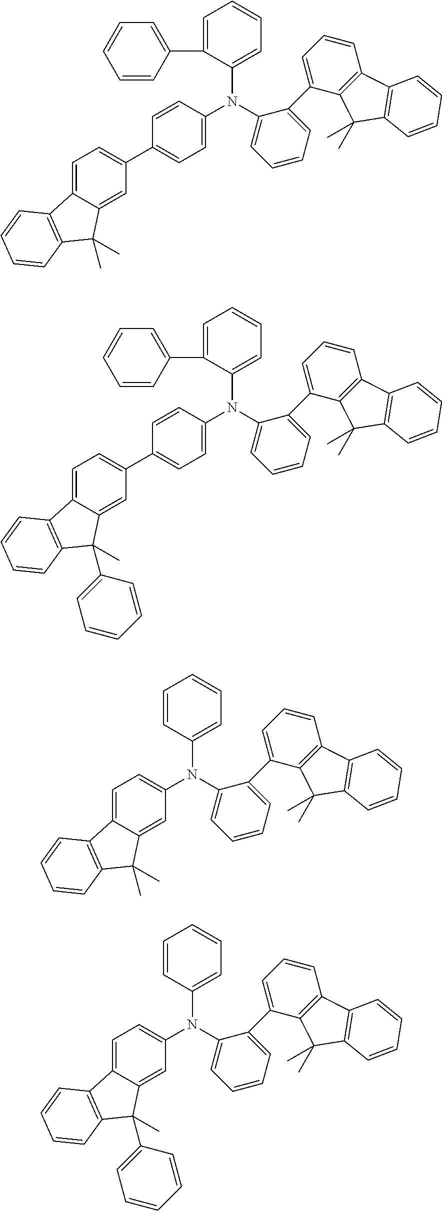 [SMILES: CC1(C)c2ccccc2-c2ccc(-c3ccc(N(c4ccccc4-c4ccccc4)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccccc3-c3ccccc3)c21 ; CC1(C)c2ccccc2-c2ccc(N(c3ccccc3)c3ccccc3-c3cccc4c3C(C)(C)c3ccccc3-4)cc21 ; CC1(C)c2ccccc2-c2cccc(-c3ccccc3N(c3ccccc3)c3ccc4c(c3)C(C)(c3ccccc3)c3ccccc3-4)c21]

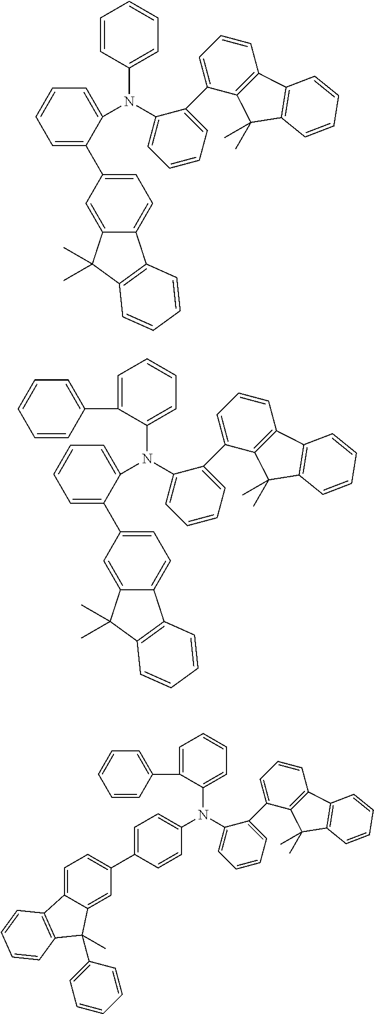

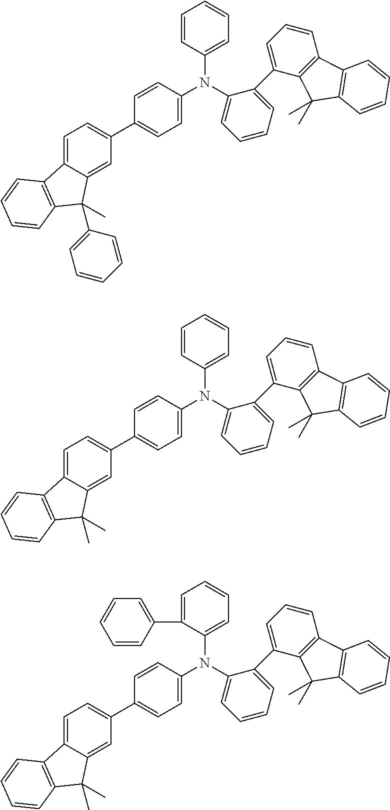 [SMILES: CC1(C)c2ccccc2-c2cccc(-c3ccccc3N(c3ccccc3)c3ccc(-c4ccc5c(c4)C(C)(c4ccccc4)c4ccccc4-5)cc3)c21 ; CC1(C)c2ccccc2-c2ccc(-c3ccc(N(c4ccccc4)c4ccccc4-c4cccc5c4C(C)(C)c4ccccc4-5)cc3)cc21 ; CC1(C)c2ccccc2-c2ccc(-c3ccc(N(c4ccccc4-c4ccccc4)c4ccccc4-c4cccc5c4C(C)(C)c4ccccc4-5)cc3)cc21]

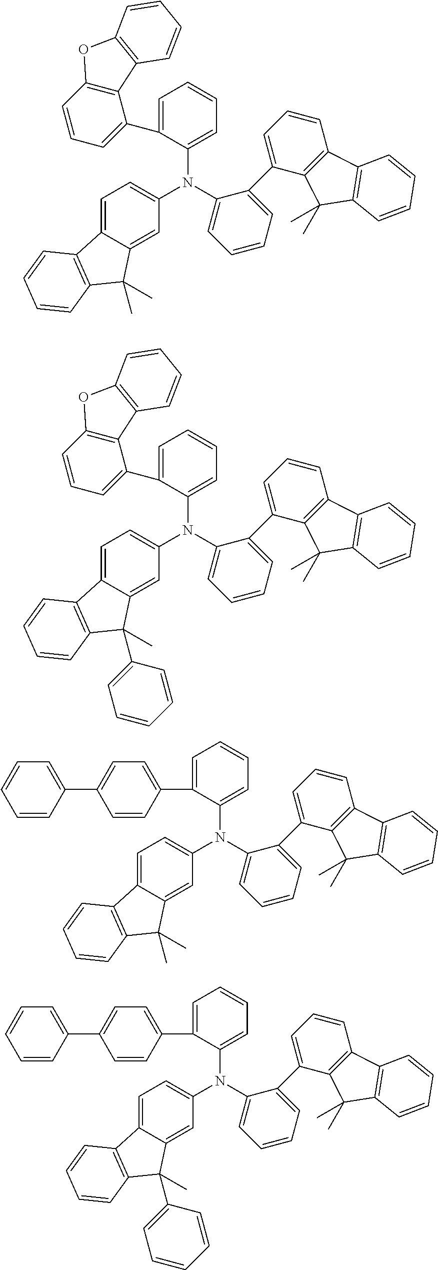

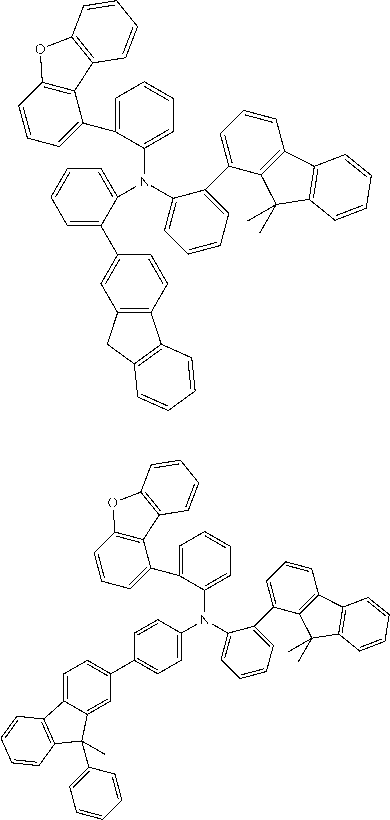 [SMILES: CC1(C)c2ccccc2-c2cccc(-c3ccccc3N(c3ccccc3-c3ccc4c(c3)Cc3ccccc3-4)c3ccccc3-c3cccc4oc5ccccc5c34)c21 ; CC1(C)c2ccccc2-c2cccc(-c3ccccc3N(c3ccc(-c4ccc5c(c4)C(C)(c4ccccc4)c4ccccc4-5)cc3)c3ccccc3-c3cccc4oc5ccccc5c34)c21]

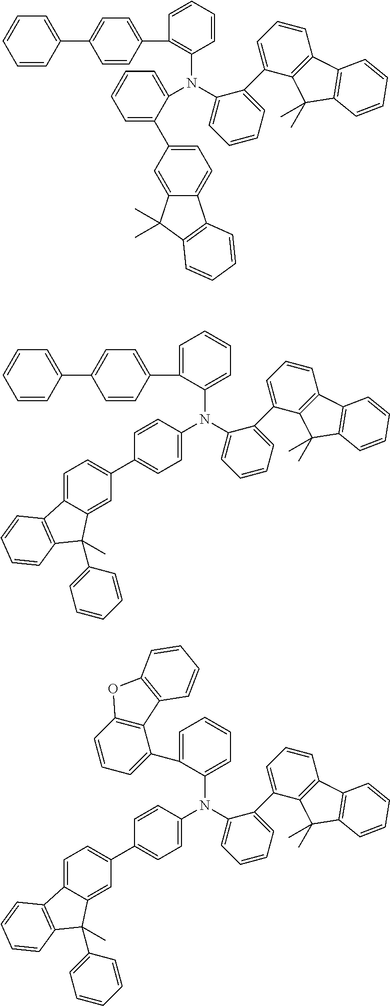 [SMILES: CC1(C)c2ccccc2-c2ccc(-c3ccccc3N(c3ccccc3-c3ccc(-c4ccccc4)cc3)c3ccccc3-c3cccc4c3C(C)(C)c3ccccc3-4)cc21 ; CC1(C)c2ccccc2-c2cccc(-c3ccccc3N(c3ccc(-c4ccc5c(c4)C(C)(c4ccccc4)c4ccccc4-5)cc3)c3ccccc3-c3ccc(-c4ccccc4)cc3)c21 ; CC1(C)c2ccccc2-c2cccc(-c3ccccc3N(c3ccc(-c4ccc5c(c4)C(C)(c4ccccc4)c4ccccc4-5)cc3)c3ccccc3-c3cccc4oc5ccccc5c34)c21]

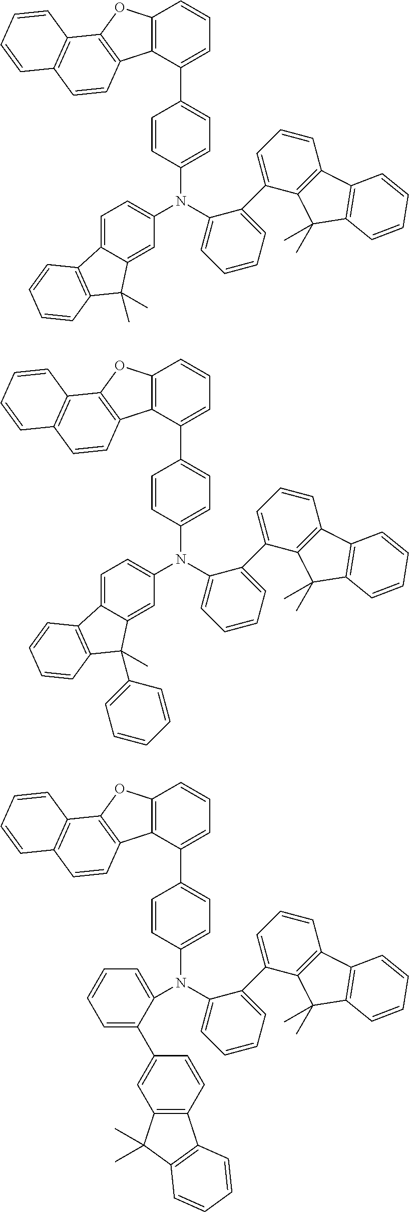

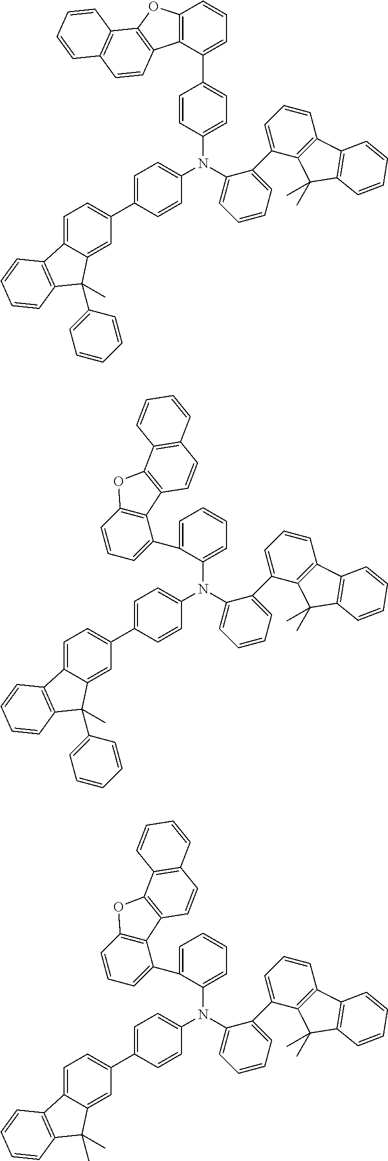 [SMILES: CC1(C)c2ccccc2-c2cccc(-c3ccccc3N(c3ccc(-c4ccc5c(c4)C(C)(c4ccccc4)c4ccccc4-5)cc3)c3ccc(-c4cccc5oc6c7ccccc7ccc6c45)cc3)c21 ; CC1(C)c2ccccc2-c2cccc(-c3ccccc3N(c3ccc(-c4ccc5c(c4)C(C)(c4ccccc4)c4ccccc4-5)cc3)c3ccccc3-c3cccc4oc5c6ccccc6ccc5c34)c21 ; CC1(C)c2ccccc2-c2ccc(-c3ccc(N(c4ccccc4-c4cccc5c4C(C)(C)c4ccccc4-5)c4ccccc4-c4cccc5oc6c7ccccc7ccc6c45)cc3)cc21]

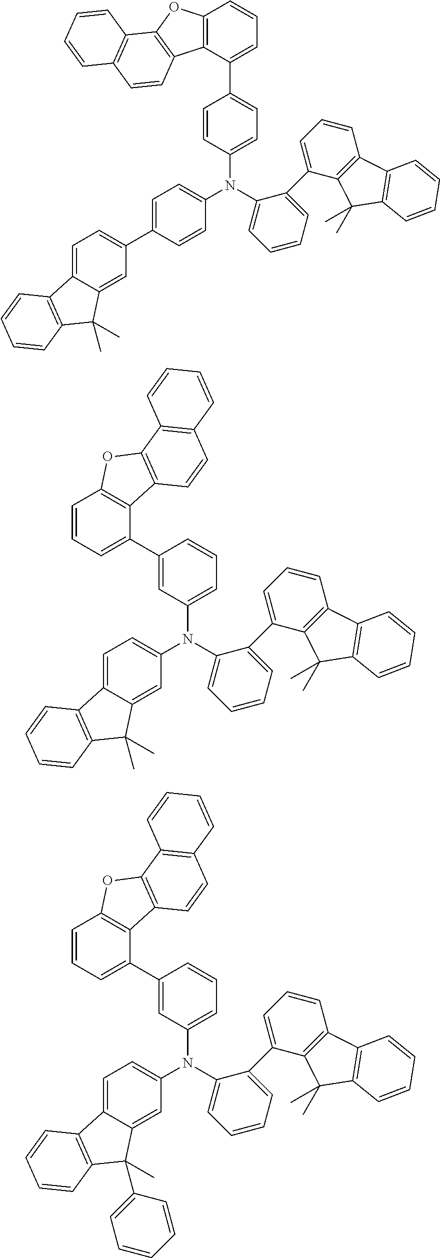

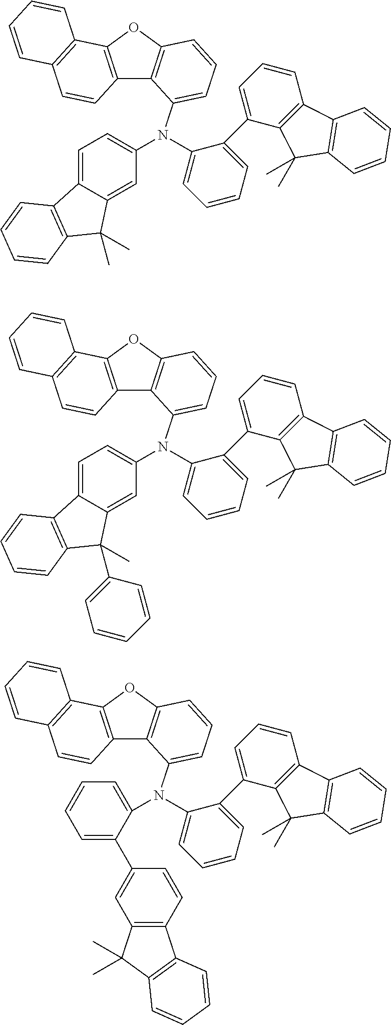

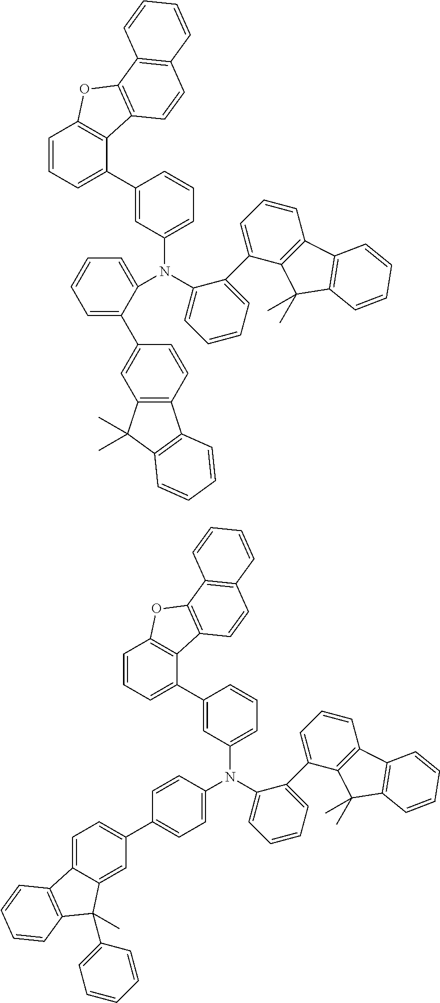

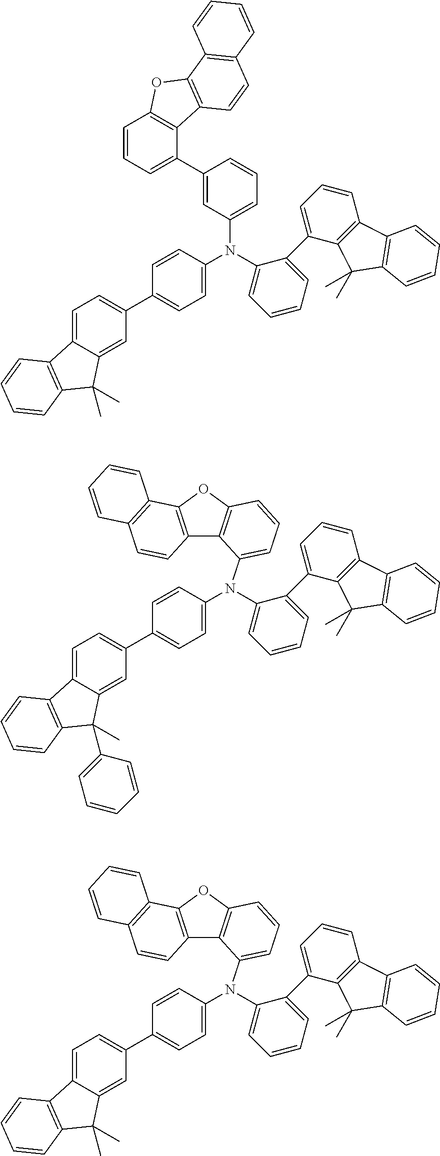 [SMILES: CC1(C)c2ccccc2-c2ccc(-c3ccc(N(c4cccc(-c5cccc6oc7c8ccccc8ccc7c56)c4)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3cccc4oc5c6ccccc6ccc5c34)c21 ; CC1(C)c2ccccc2-c2ccc(-c3ccc(N(c4ccccc4-c4cccc5c4C(C)(C)c4ccccc4-5)c4cccc5oc6c7ccccc7ccc6c45)cc3)cc21]

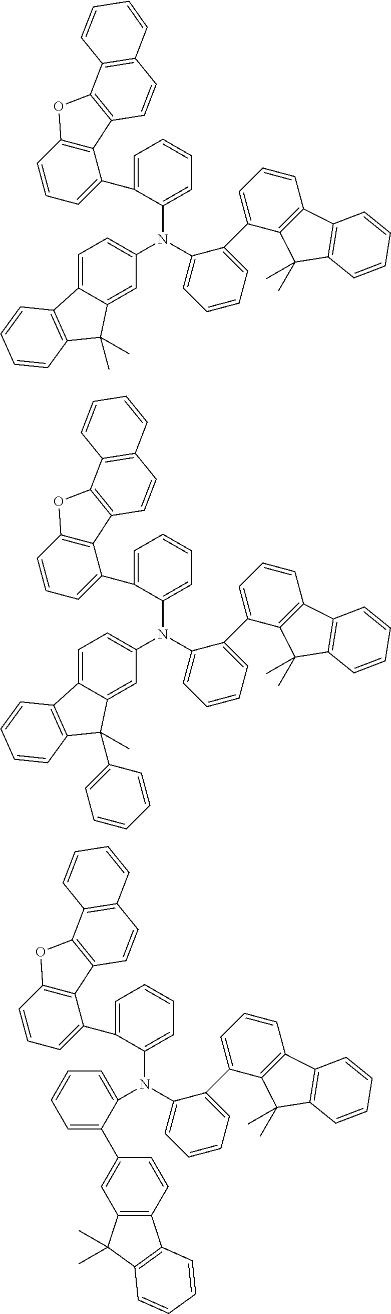

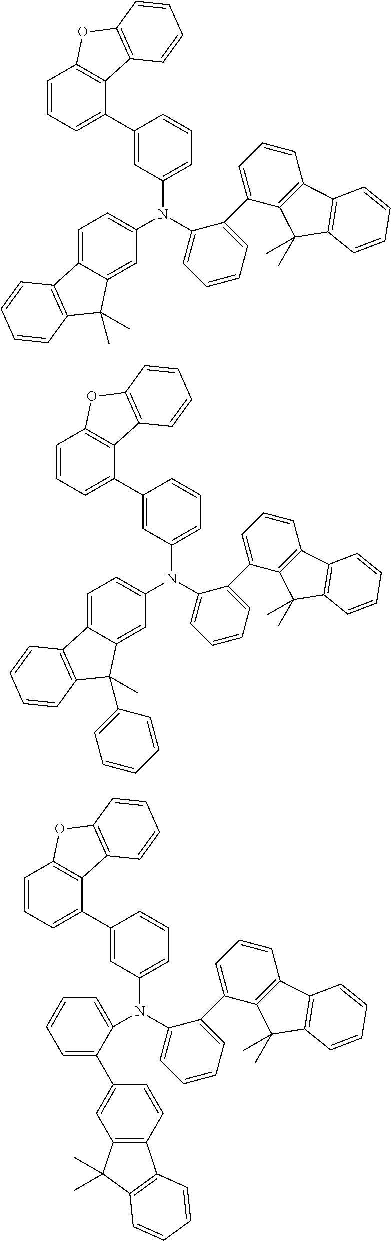

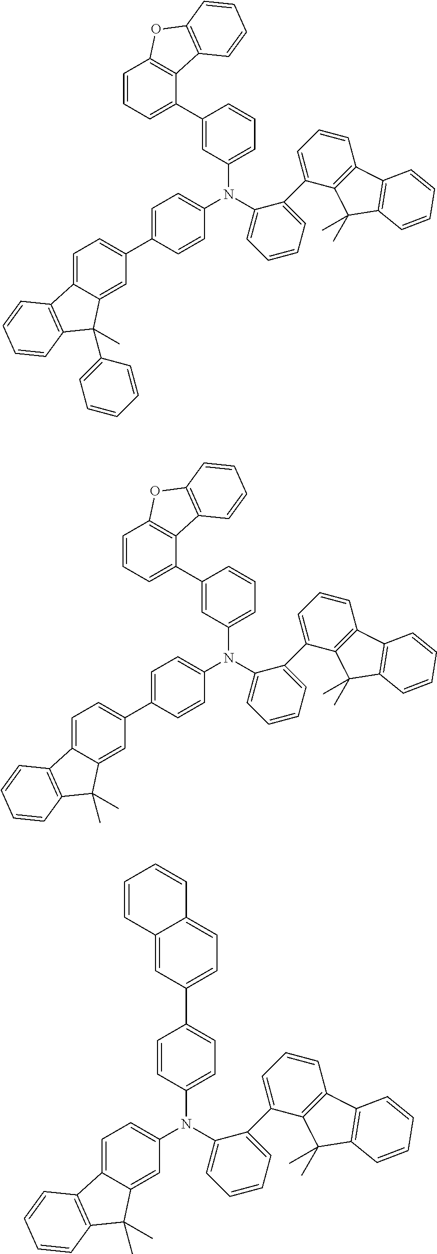 [SMILES: CC1(C)c2ccccc2-c2cccc(-c3ccccc3N(c3ccc(-c4ccc5c(c4)C(C)(c4ccccc4)c4ccccc4-5)cc3)c3cccc(-c4cccc5oc6ccccc6c45)c3)c21 ; CC1(C)c2ccccc2-c2ccc(-c3ccc(N(c4cccc(-c5cccc6oc7ccccc7c56)c4)c4ccccc4-c4cccc5c4C(C)(C)c4ccccc4-5)cc3)cc21 ; CC1(C)c2ccccc2-c2ccc(N(c3ccc(-c4ccc5ccccc5c4)cc3)c3ccccc3-c3cccc4c3C(C)(C)c3ccccc3-4)cc21]

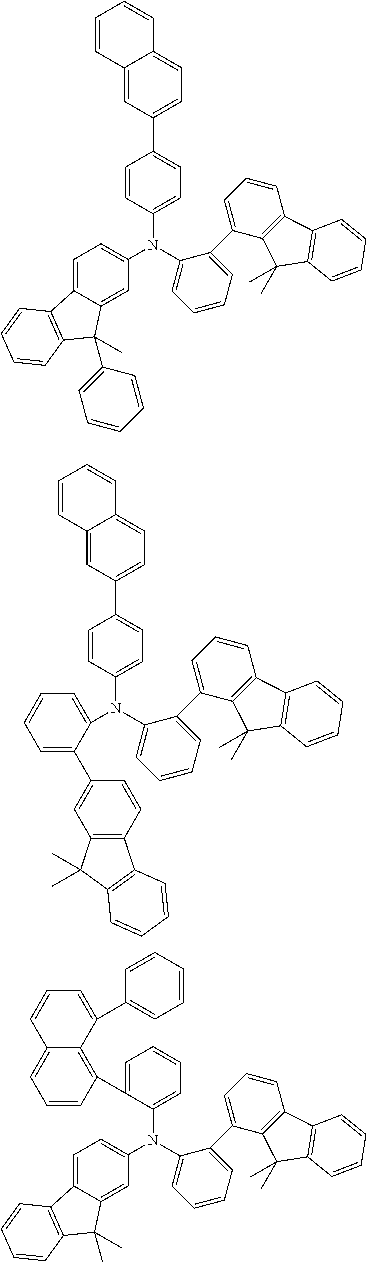

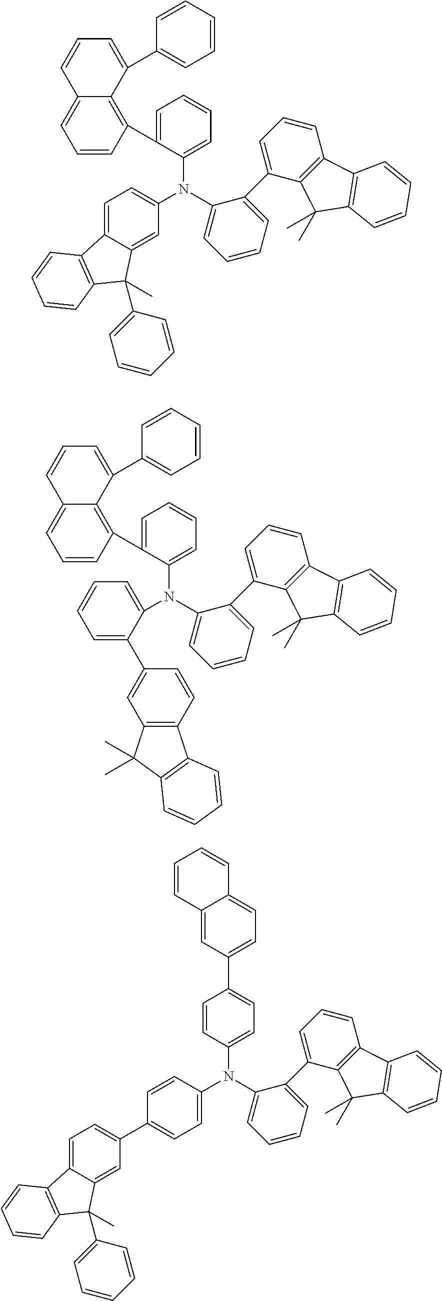

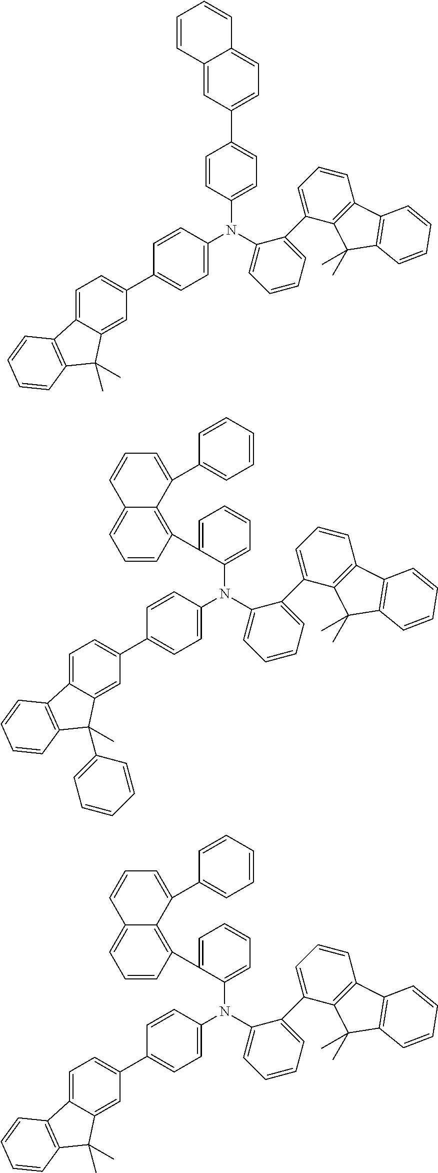 [SMILES: CC1(C)c2ccccc2-c2ccc(-c3ccc(N(c4ccc(-c5ccc6ccccc6c5)cc4)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccccc3-c3cccc4cccc(-c5ccccc5)c34)c21 ; CC1(C)c2ccccc2-c2ccc(-c3ccc(N(c4ccccc4-c4cccc5c4C(C)(C)c4ccccc4-5)c4ccccc4-c4cccc5cccc(-c6ccccc6)c45)cc3)cc21]

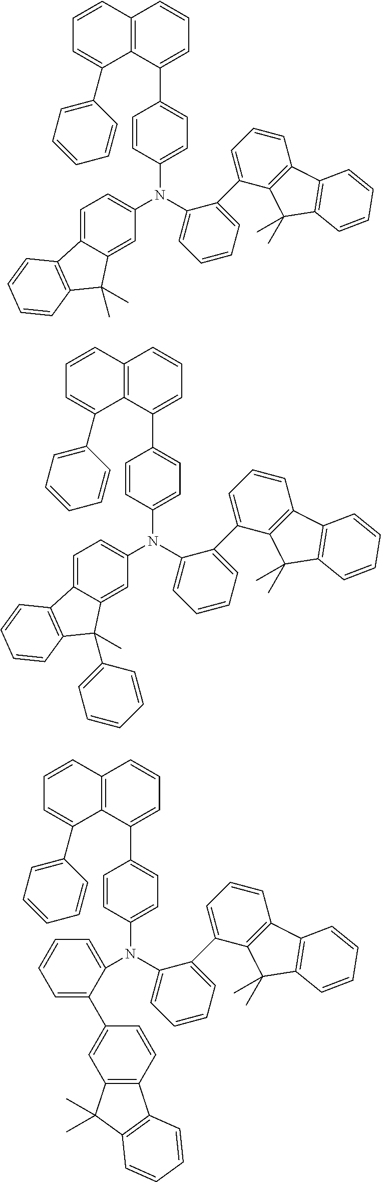

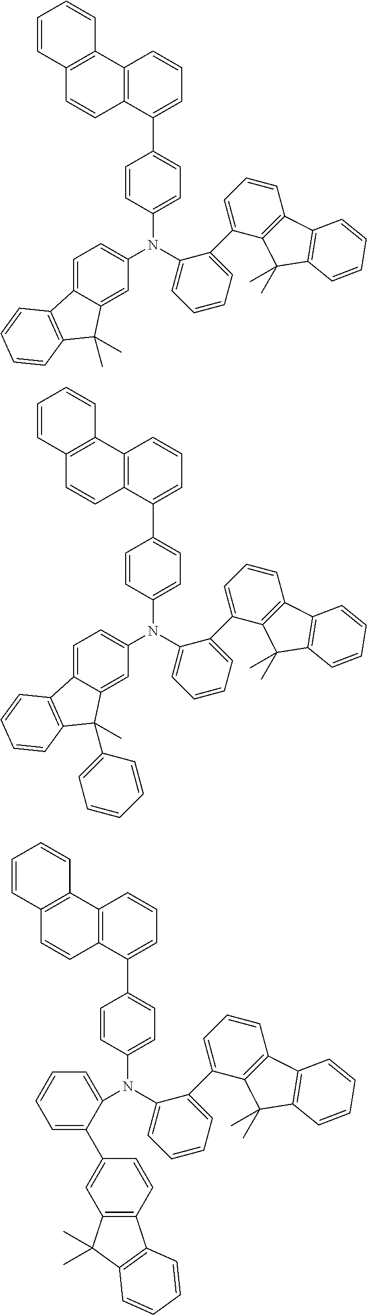

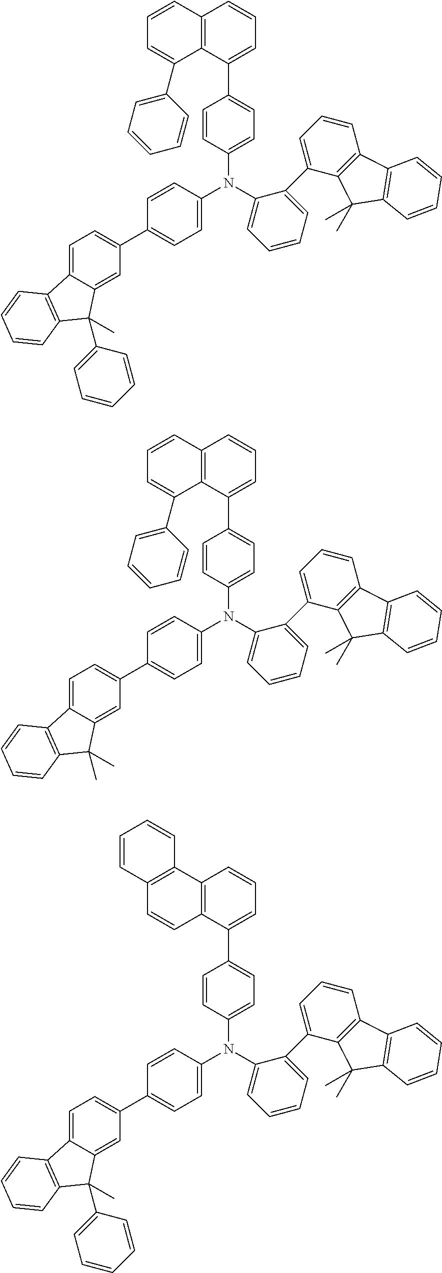 [SMILES: CC1(C)c2ccccc2-c2cccc(-c3ccccc3N(c3ccc(-c4ccc5c(c4)C(C)(c4ccccc4)c4ccccc4-5)cc3)c3ccc(-c4cccc5cccc(-c6ccccc6)c45)cc3)c21 ; CC1(C)c2ccccc2-c2ccc(-c3ccc(N(c4ccc(-c5cccc6cccc(-c7ccccc7)c56)cc4)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccc(-c4cccc5c4ccc4ccccc45)cc3)c21]

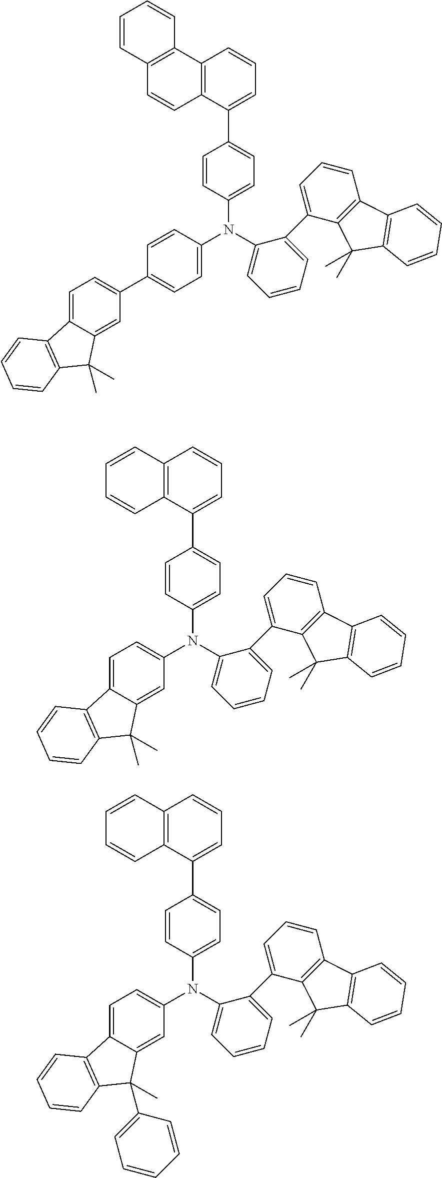

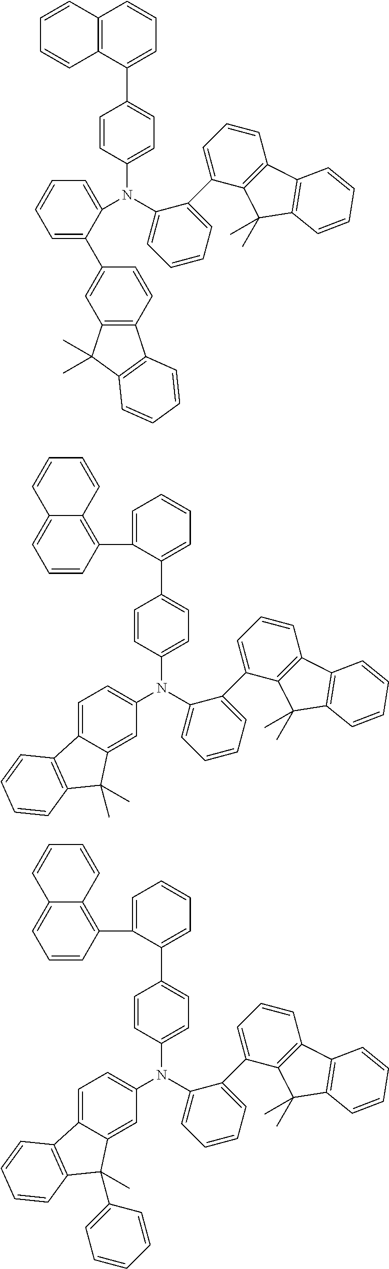

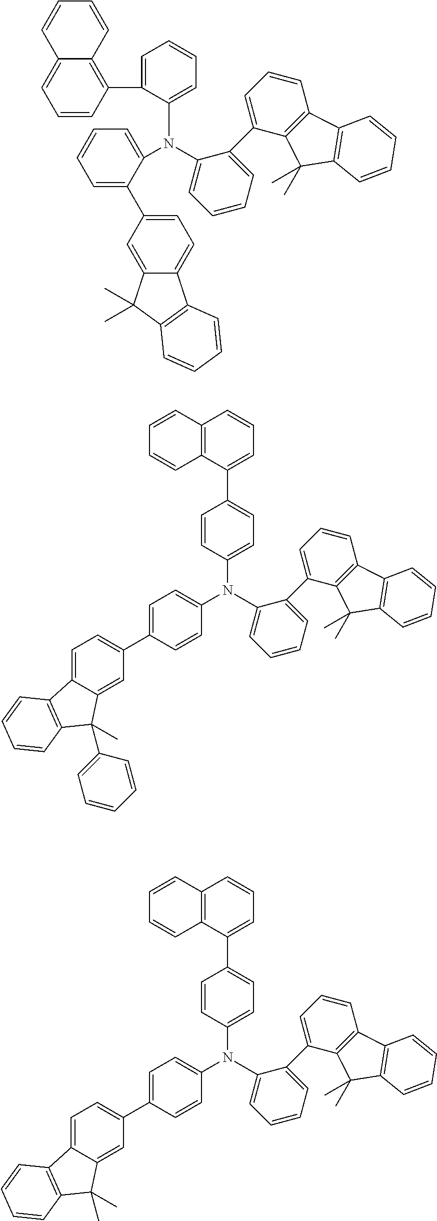 [SMILES: CC1(C)c2ccccc2-c2ccc(-c3ccccc3N(c3ccccc3-c3cccc4c3C(C)(C)c3ccccc3-4)c3ccccc3-c3cccc4ccccc34)cc21 ; CC1(C)c2ccccc2-c2cccc(-c3ccccc3N(c3ccc(-c4ccc5c(c4)C(C)(c4ccccc4)c4ccccc4-5)cc3)c3ccc(-c4cccc5ccccc45)cc3)c21 ; CC1(C)c2ccccc2-c2ccc(-c3ccc(N(c4ccc(-c5cccc6ccccc56)cc4)c4ccccc4-c4cccc5c4C(C)(C)c4ccccc4-5)cc3)cc21]

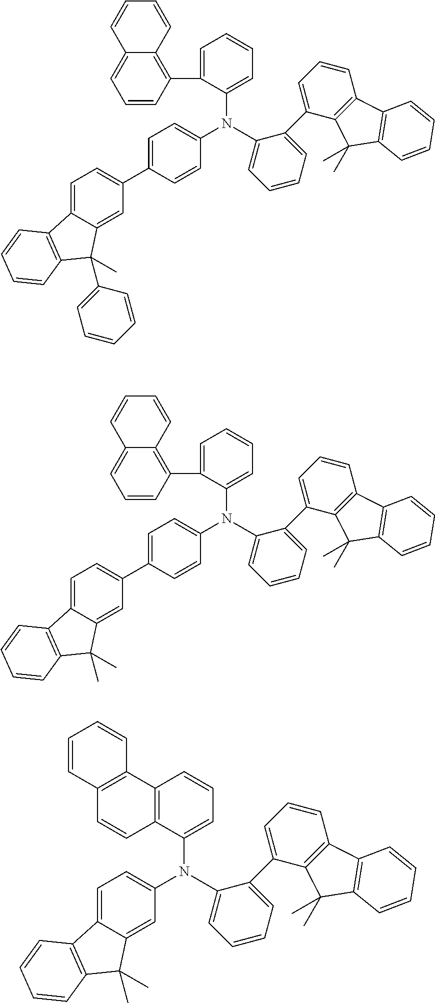

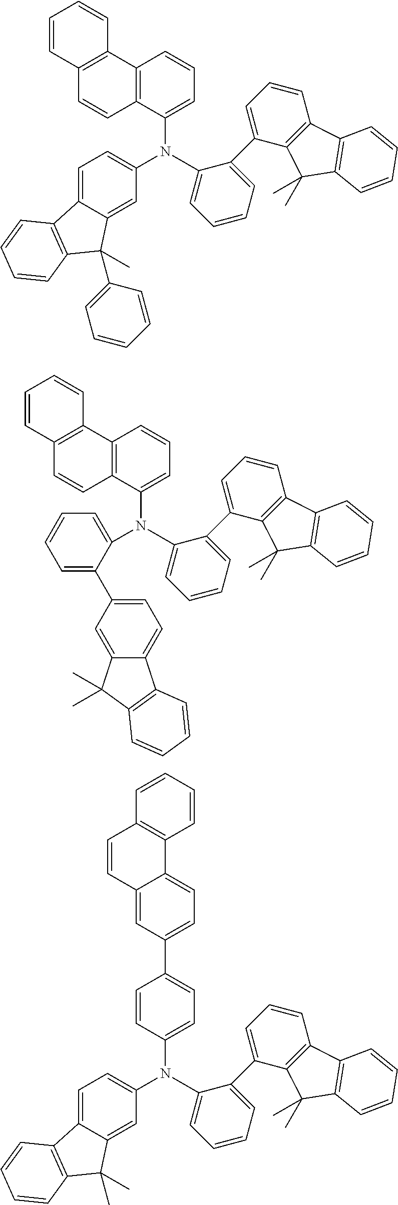

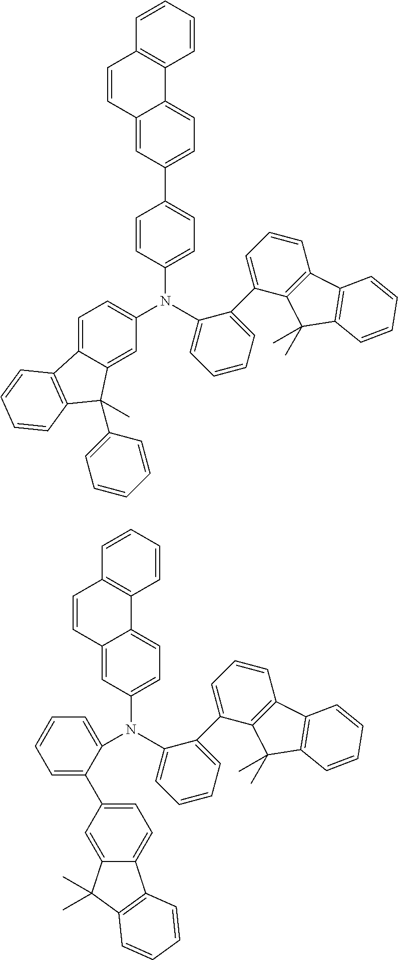

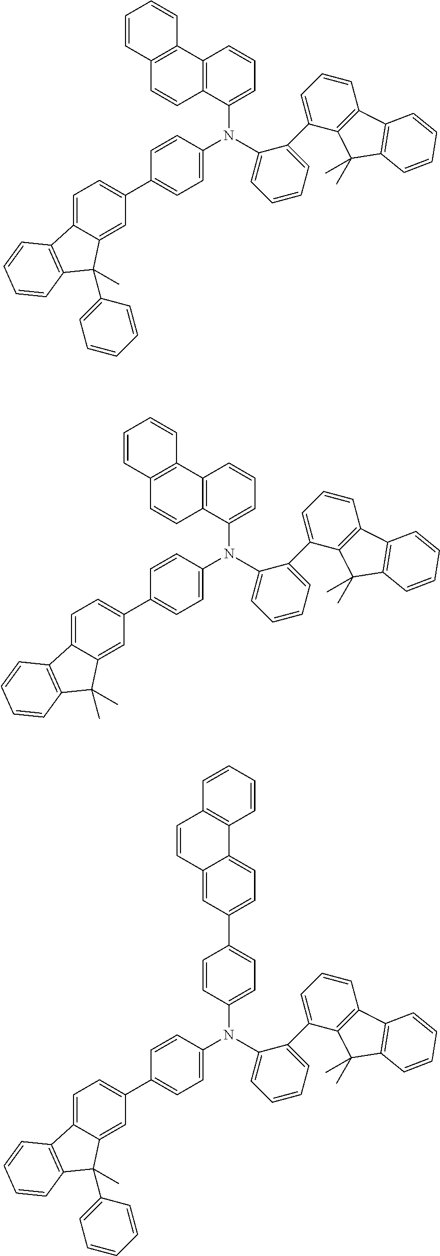 [SMILES: CC1(C)c2ccccc2-c2cccc(-c3ccccc3N(c3ccc(-c4ccc5c(c4)C(C)(c4ccccc4)c4ccccc4-5)cc3)c3cccc4c3ccc3ccccc34)c21 ; CC1(C)c2ccccc2-c2ccc(-c3ccc(N(c4ccccc4-c4cccc5c4C(C)(C)c4ccccc4-5)c4cccc5c4ccc4ccccc45)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccc(-c4ccc5c(ccc6ccccc65)c4)cc3)c21]

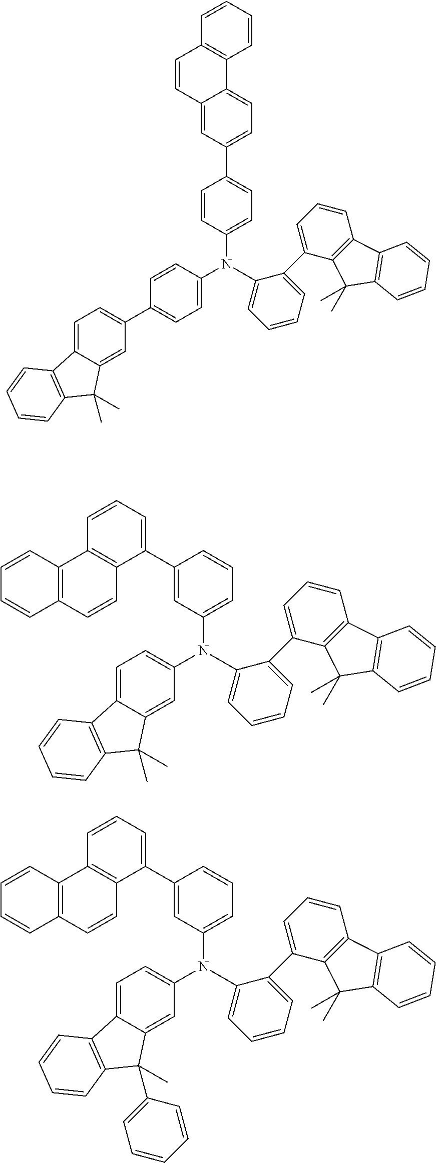

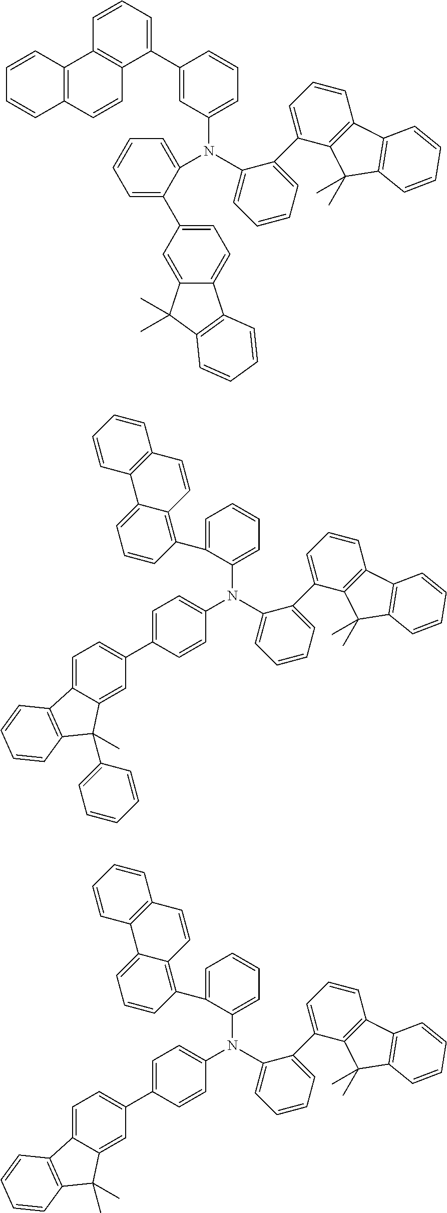

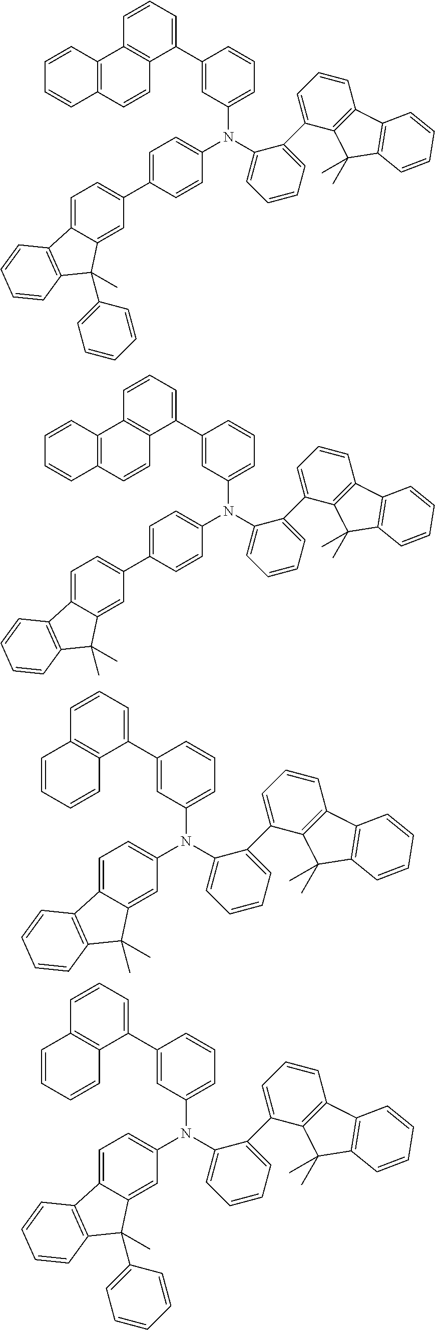

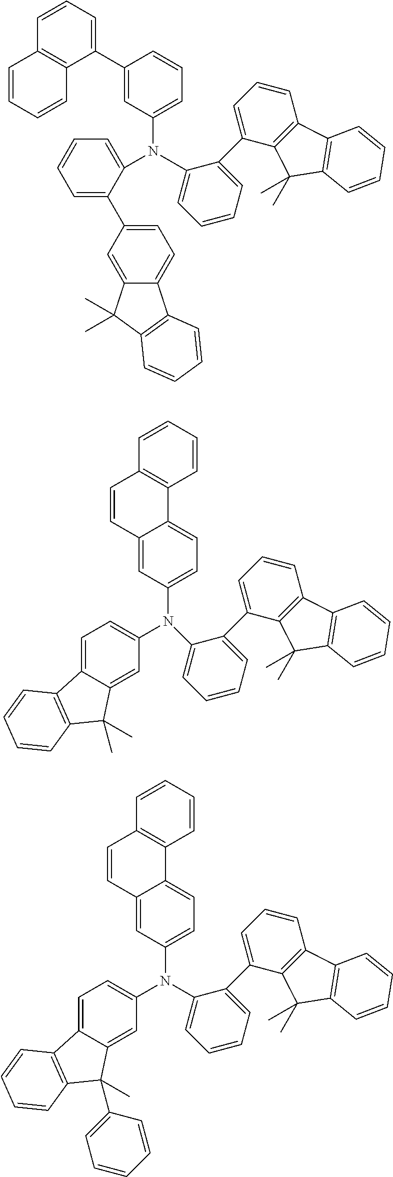

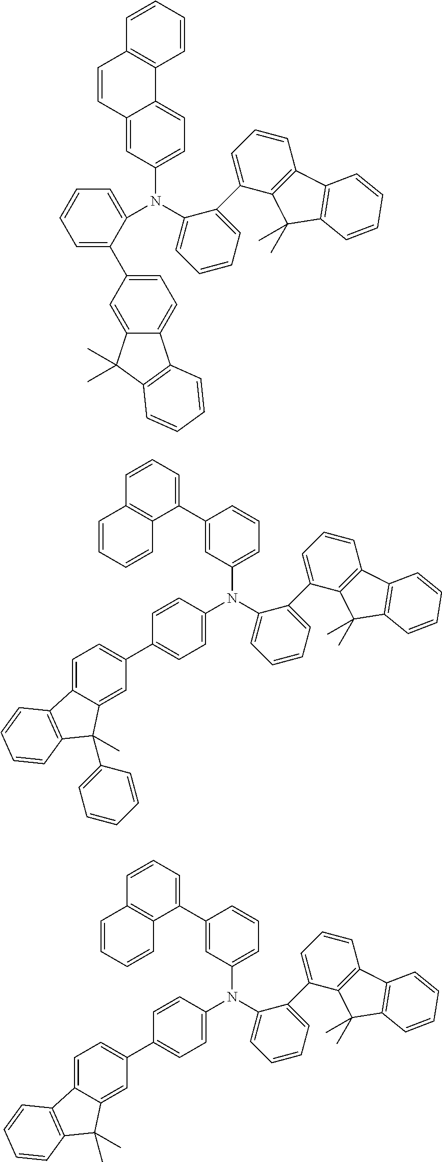

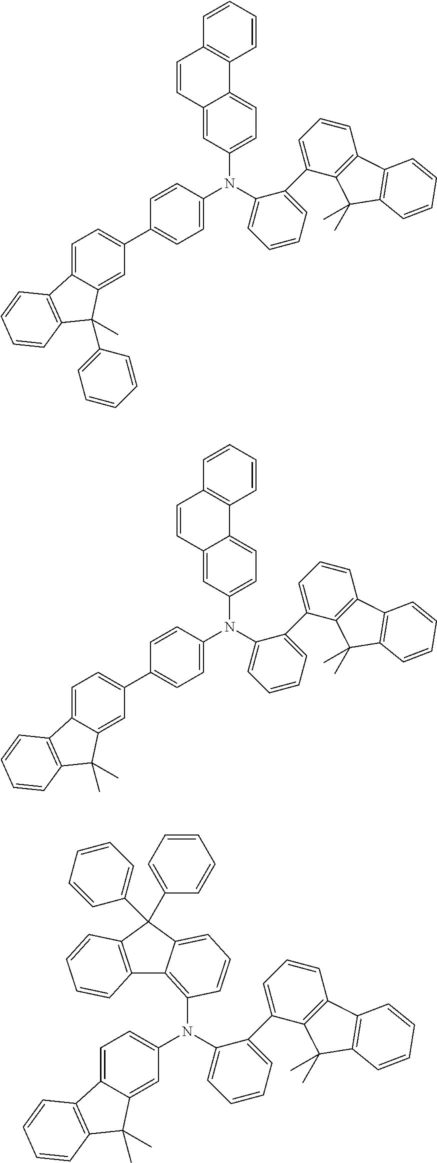

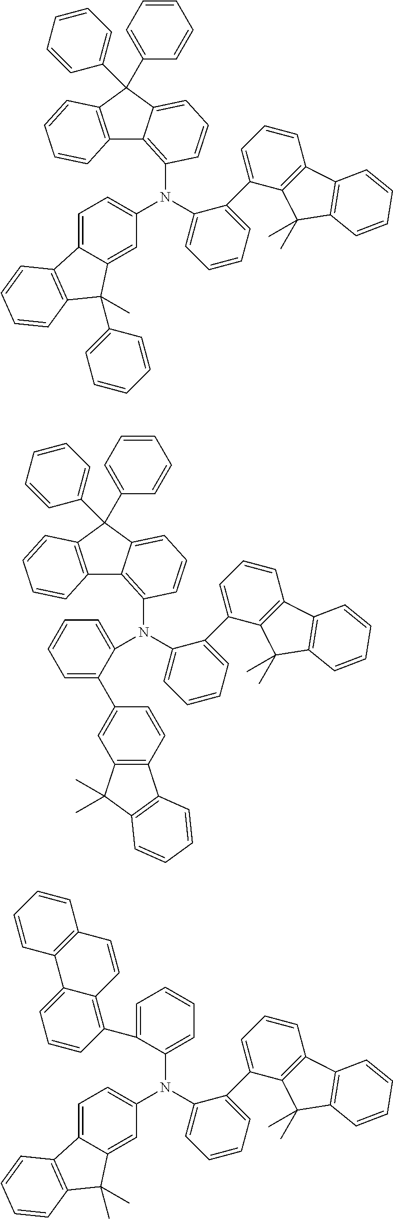

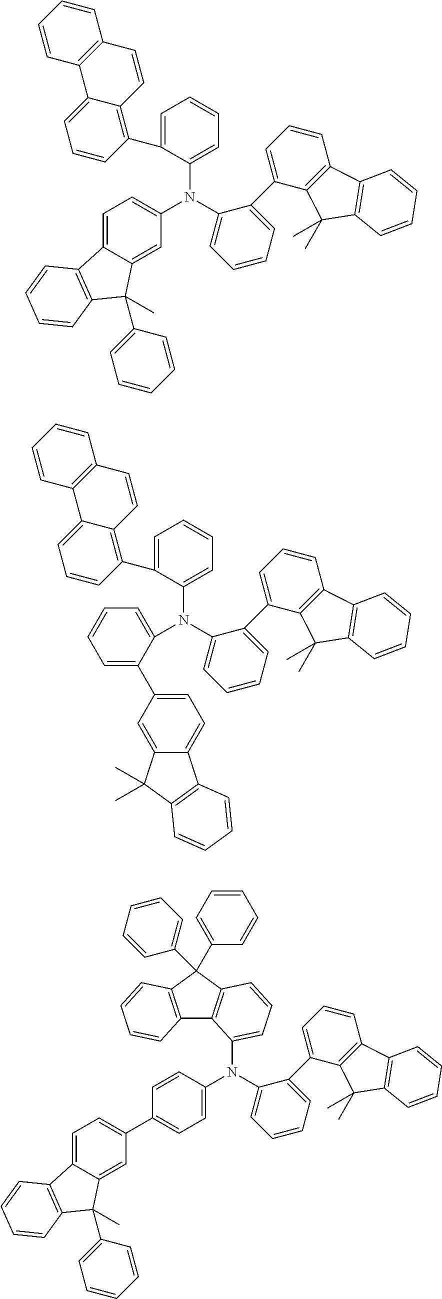

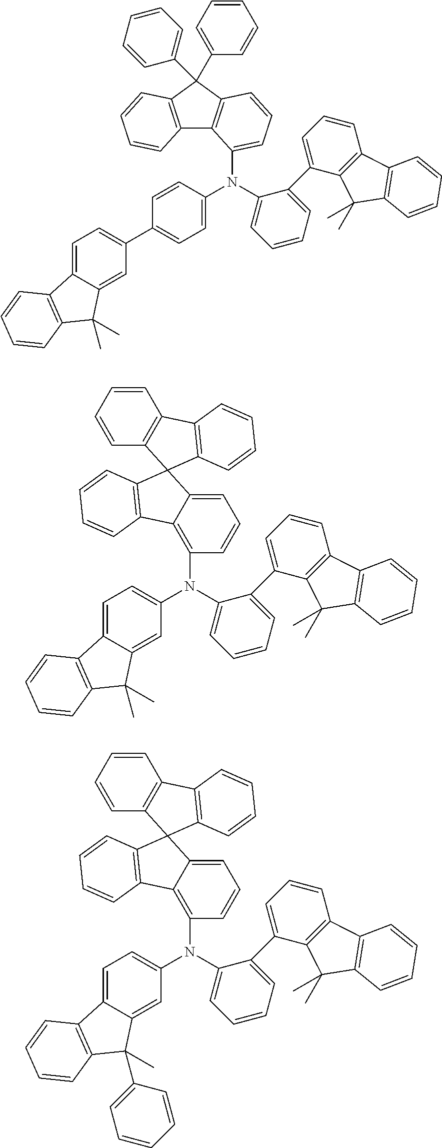

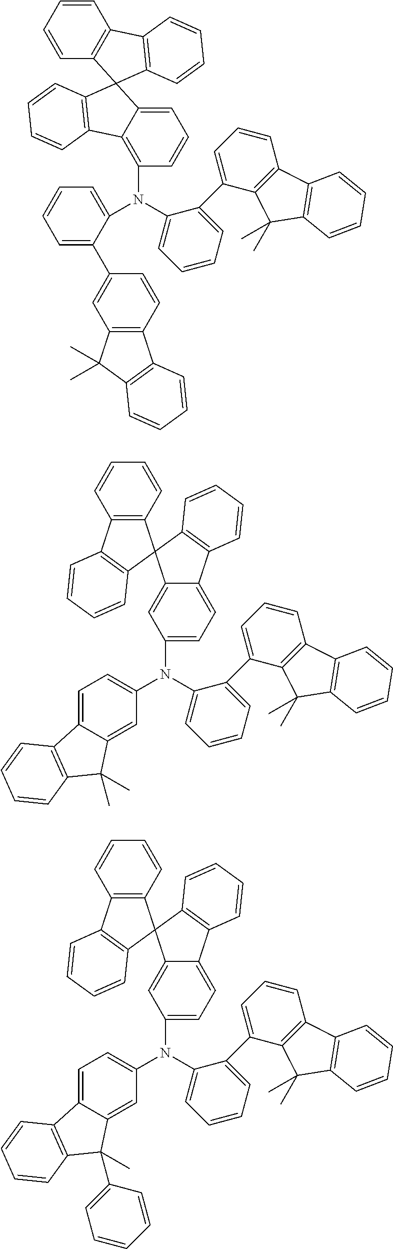

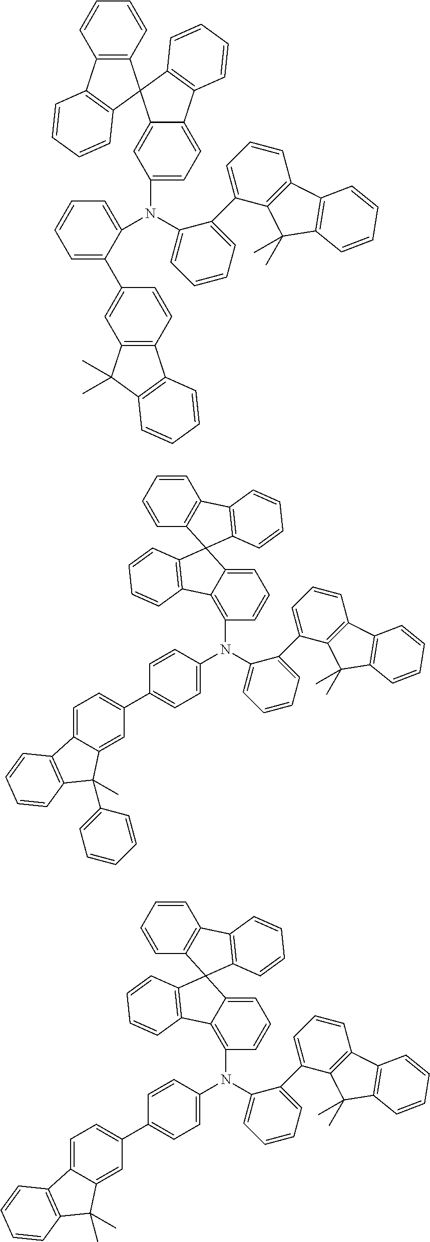

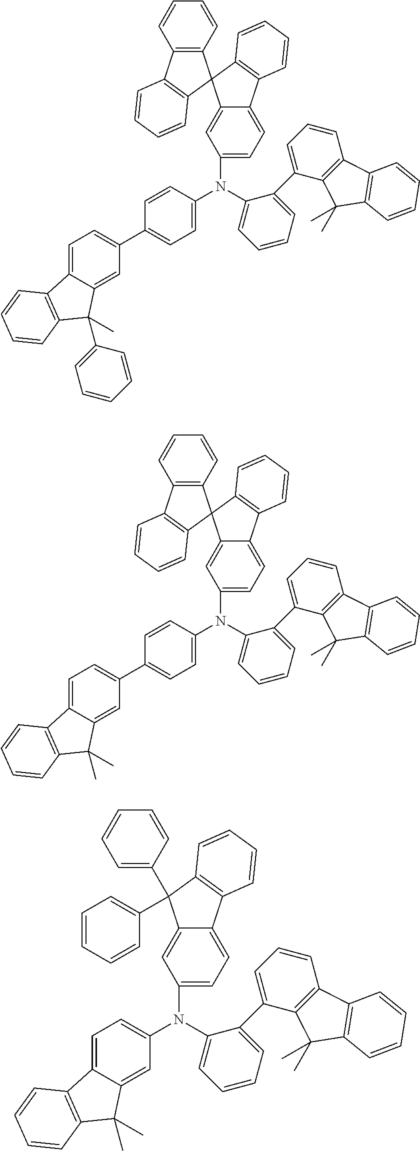

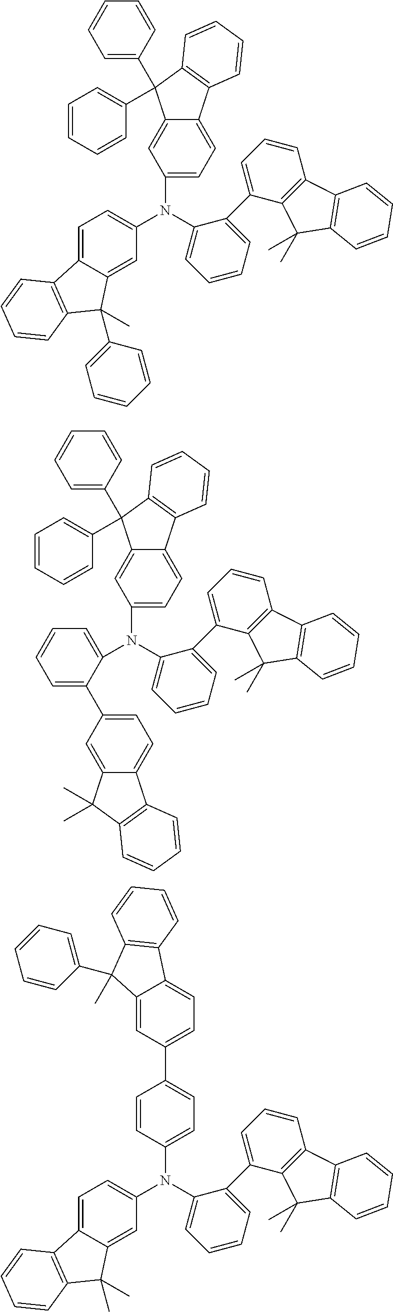

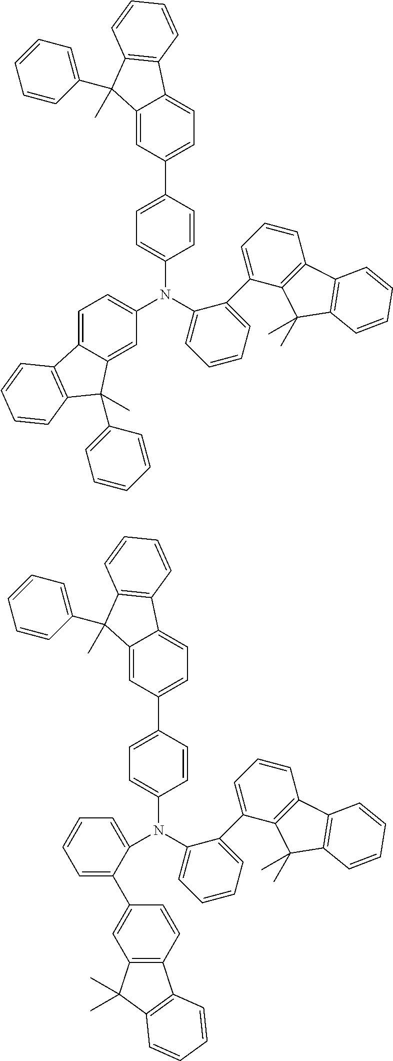

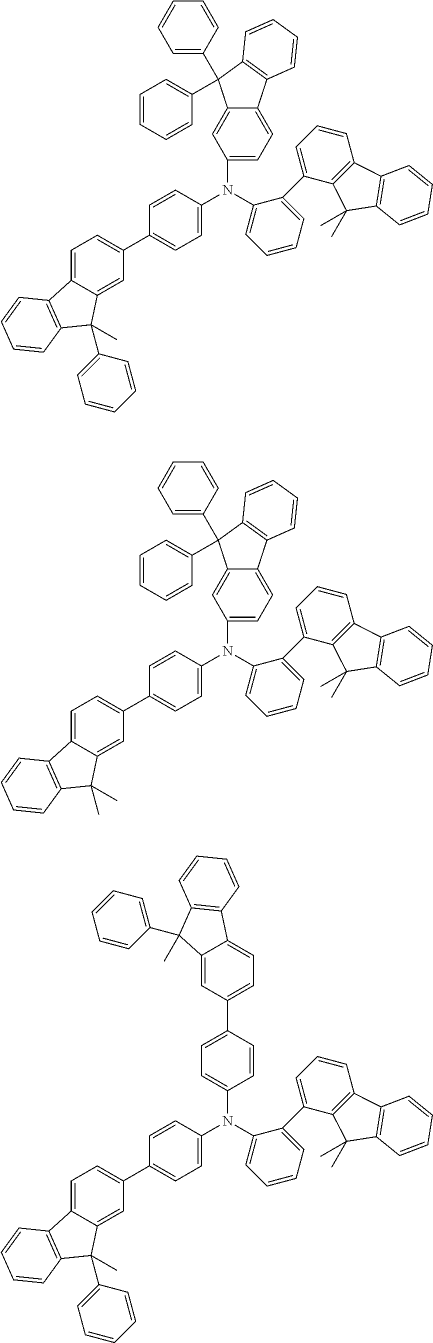 [SMILES: CC1(C)c2ccccc2-c2cccc(-c3ccccc3N(c3ccc(-c4ccc5c(c4)C(C)(c4ccccc4)c4ccccc4-5)cc3)c3ccc4c(c3)C(c3ccccc3)(c3ccccc3)c3ccccc3-4)c21 ; CC1(C)c2ccccc2-c2ccc(-c3ccc(N(c4ccc5c(c4)C(c4ccccc4)(c4ccccc4)c4ccccc4-5)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccc(-c4ccc5c(c4)C(C)(c4ccccc4)c4ccccc4-5)cc3)c21]

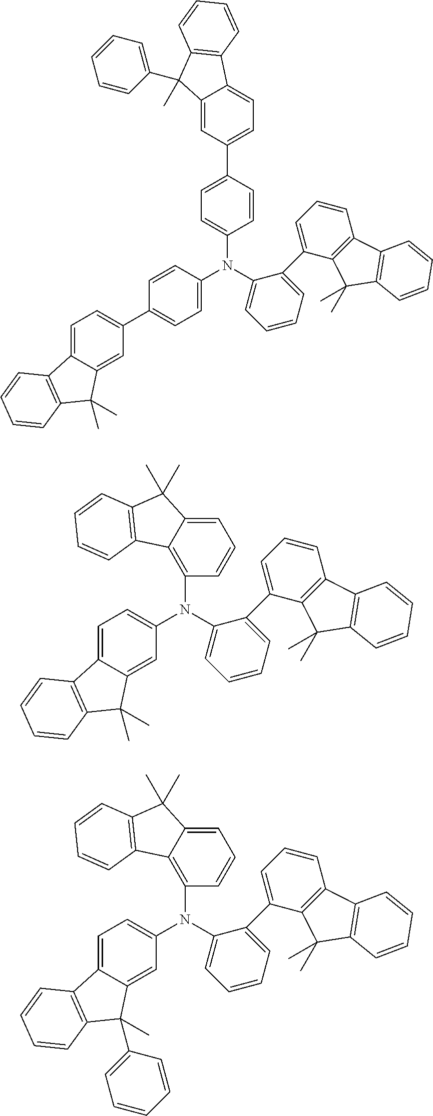

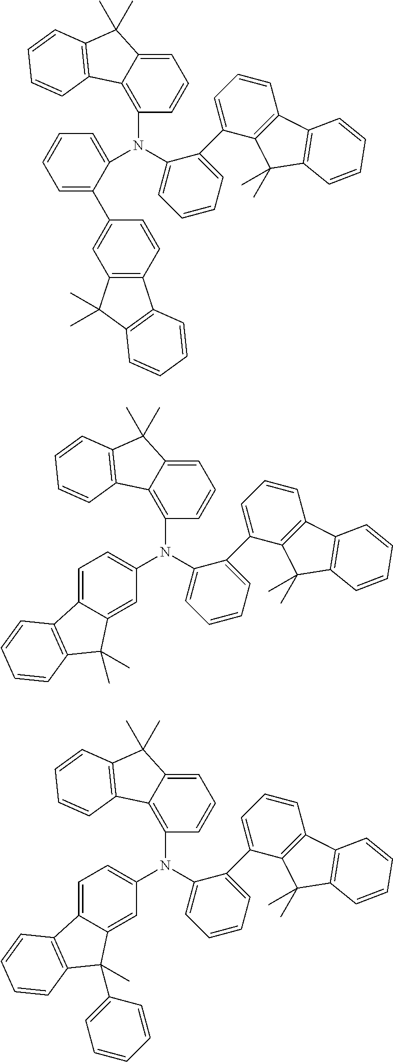

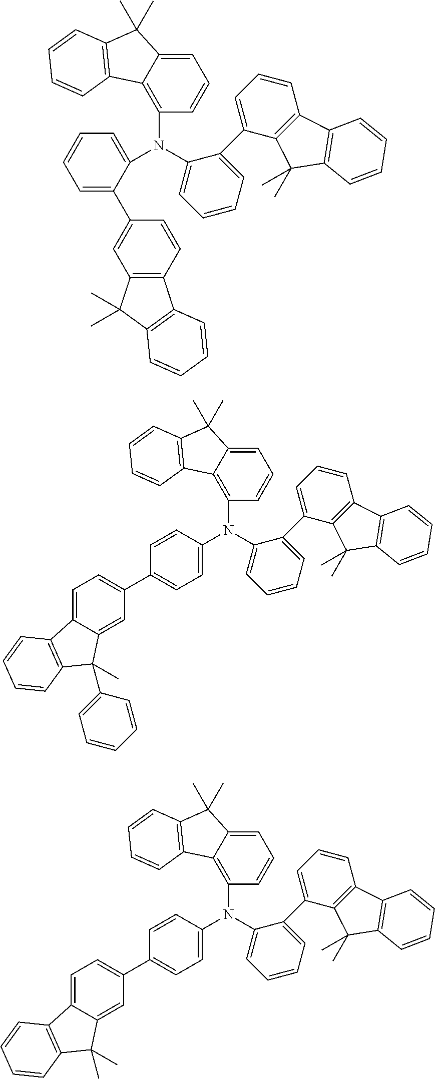

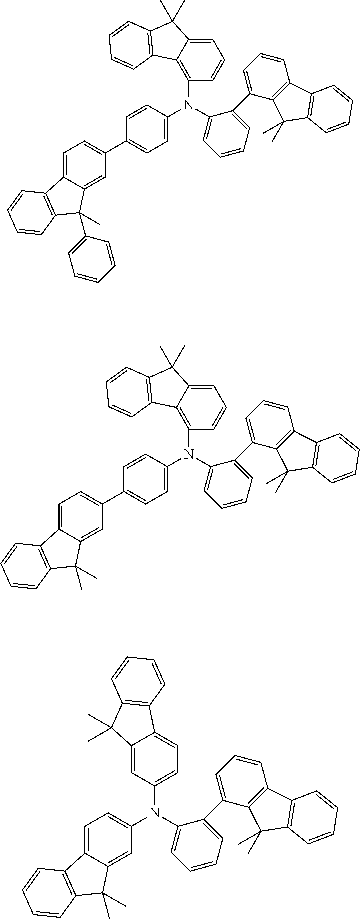 [SMILES: CC1(C)c2ccccc2-c2c(N(c3ccc(-c4ccc5c(c4)C(C)(c4ccccc4)c4ccccc4-5)cc3)c3ccccc3-c3cccc4c3C(C)(C)c3ccccc3-4)cccc21 ; CC1(C)c2ccccc2-c2ccc(-c3ccc(N(c4ccccc4-c4cccc5c4C(C)(C)c4ccccc4-5)c4cccc5c4-c4ccccc4C5(C)C)cc3)cc21 ; CC1(C)c2ccccc2-c2ccc(N(c3ccc4c(c3)C(C)(C)c3ccccc3-4)c3ccccc3-c3cccc4c3C(C)(C)c3ccccc3-4)cc21]

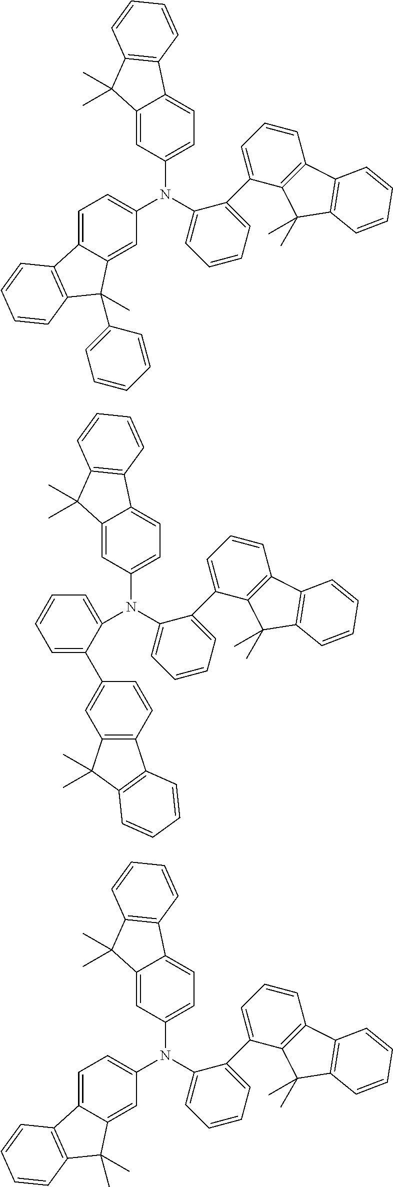

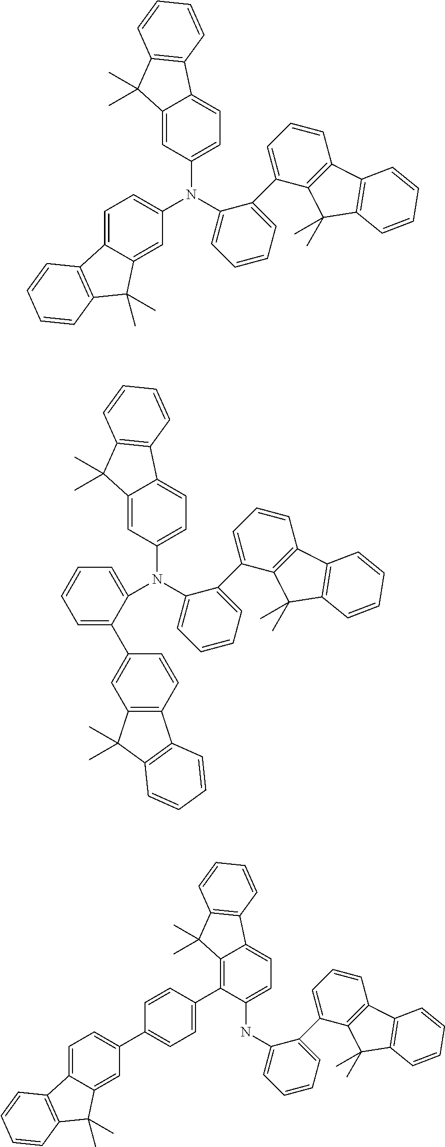 [SMILES: CC1(C)c2ccccc2-c2ccc(N(c3ccc4c(c3)C(C)(C)c3ccccc3-4)c3ccccc3-c3cccc4c3C(C)(C)c3ccccc3-4)cc21 ; CC1(C)c2ccccc2-c2ccc(-c3ccccc3N(c3ccc4c(c3)C(C)(C)c3ccccc3-4)c3ccccc3-c3cccc4c3C(C)(C)c3ccccc3-4)cc21 ; CC1(C)c2ccccc2-c2ccc(-c3ccc(-c4c(Nc5ccccc5-c5cccc6c5C(C)(C)c5ccccc5-6)ccc5c4C(C)(C)c4ccccc4-5)cc3)cc21]

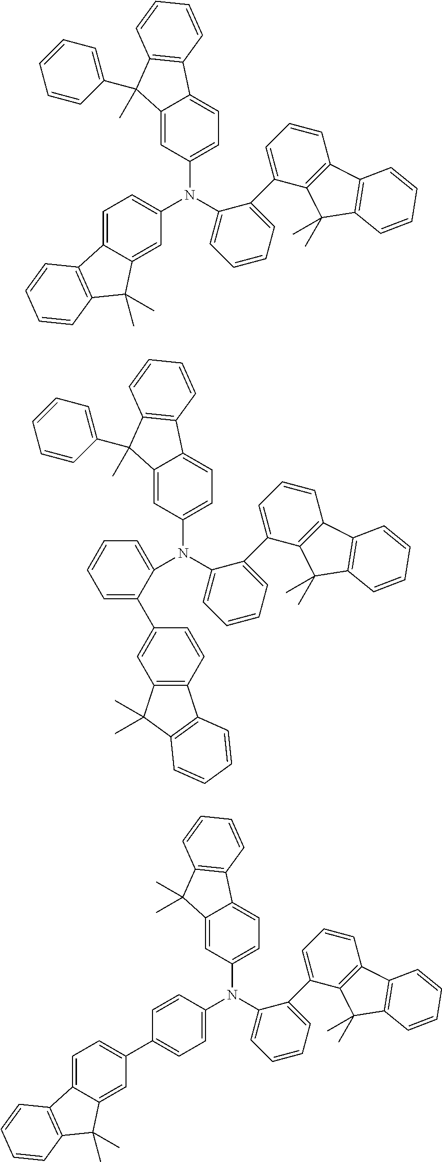

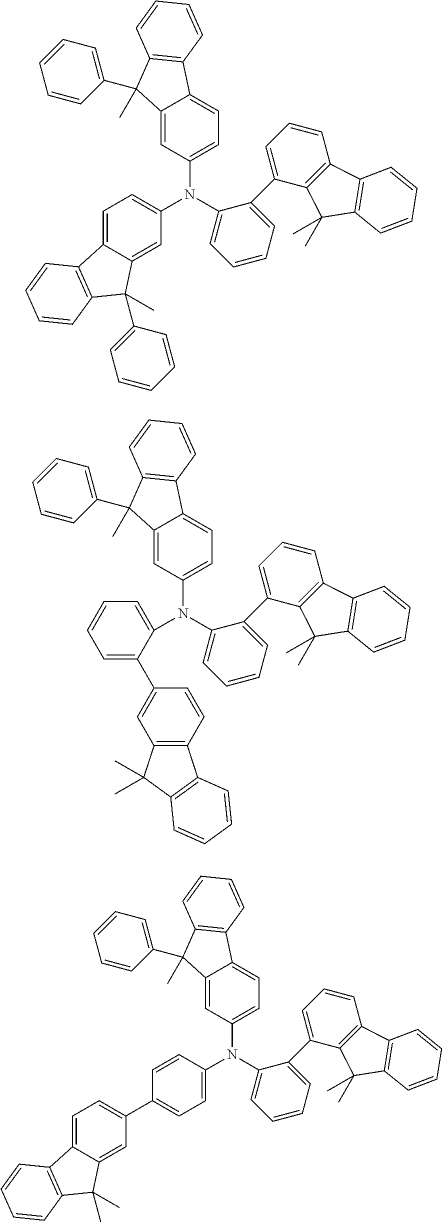

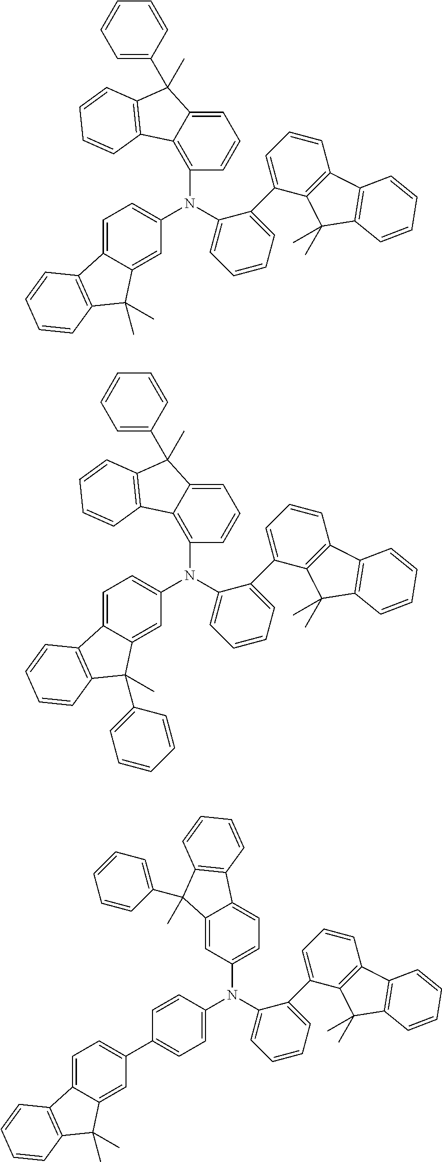

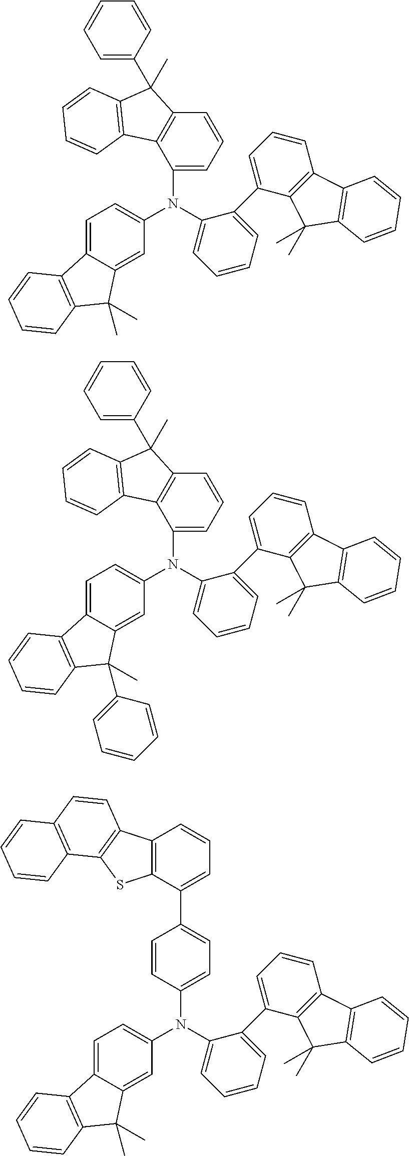

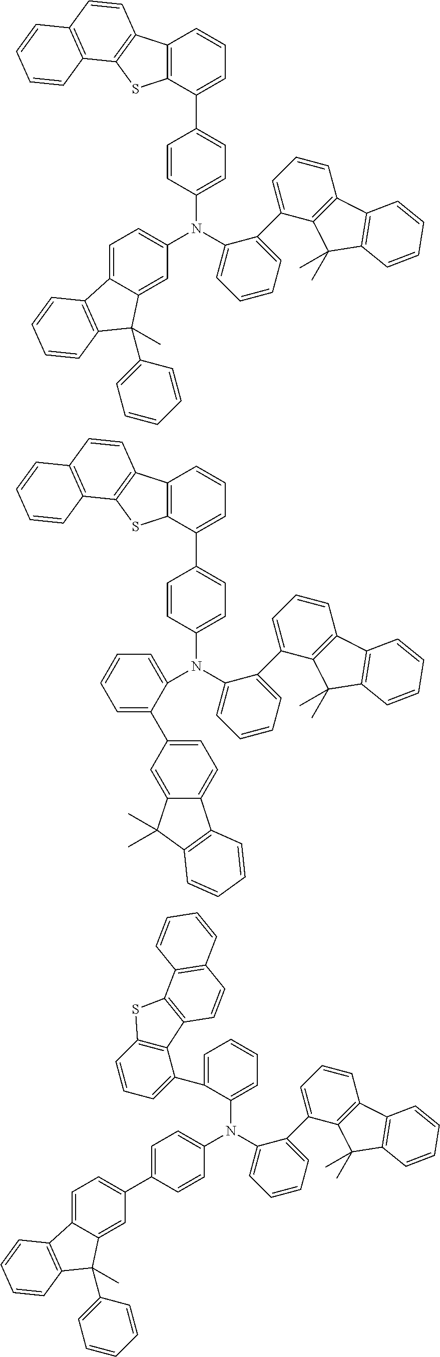

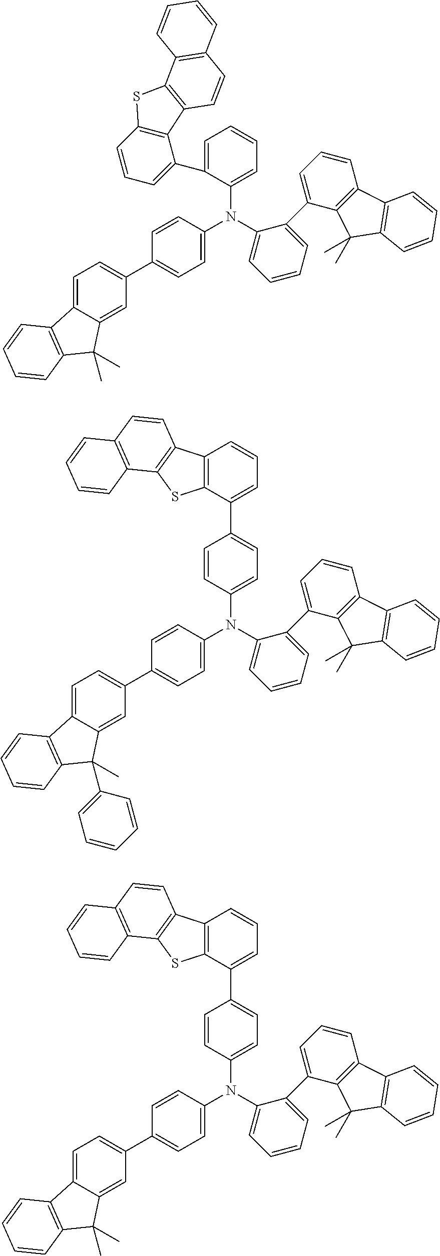

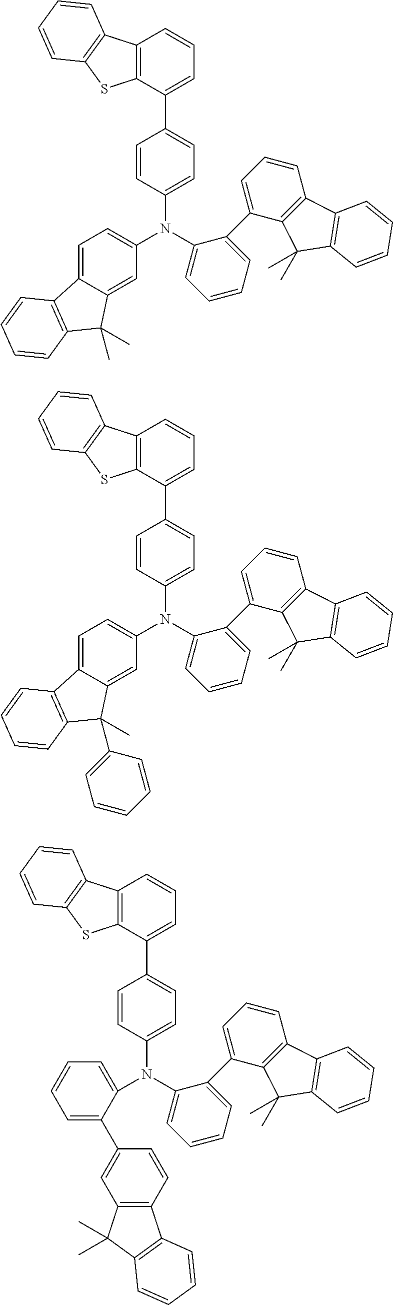

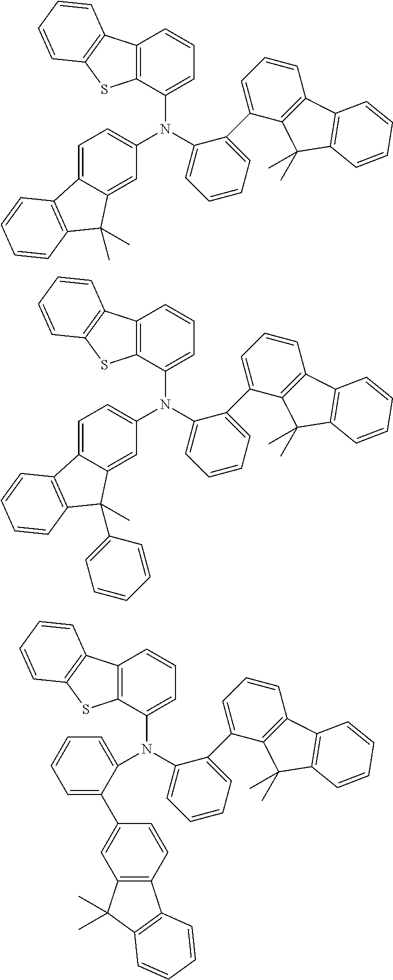

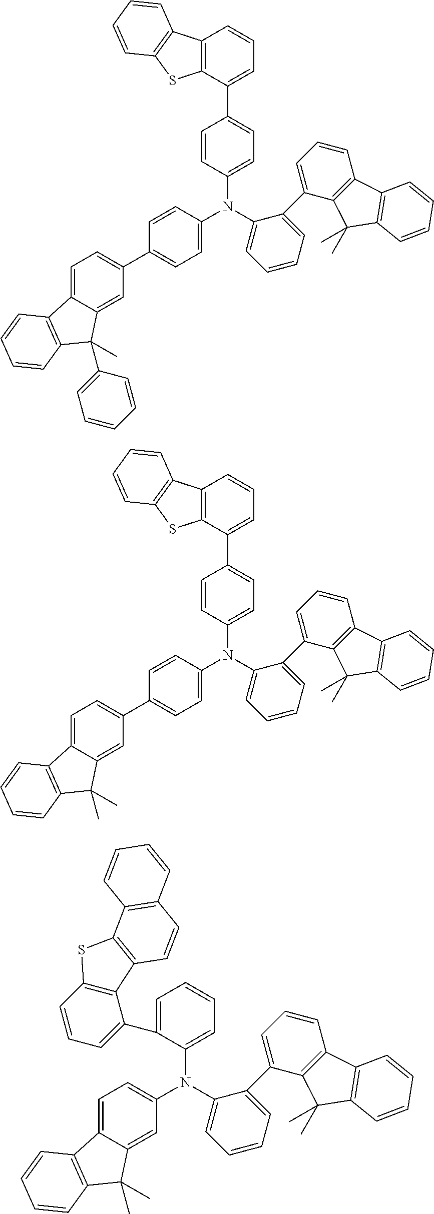

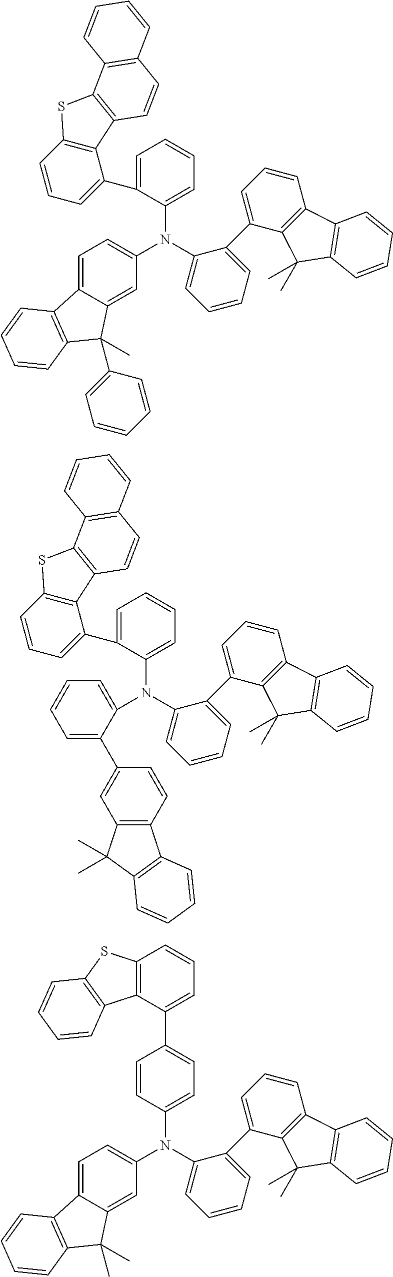

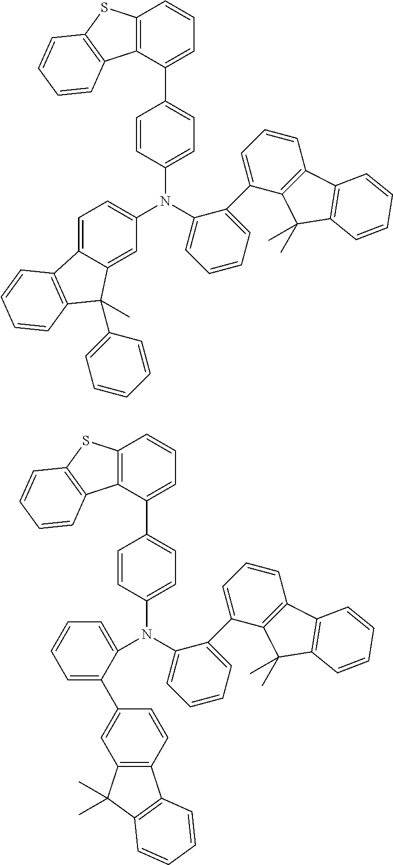

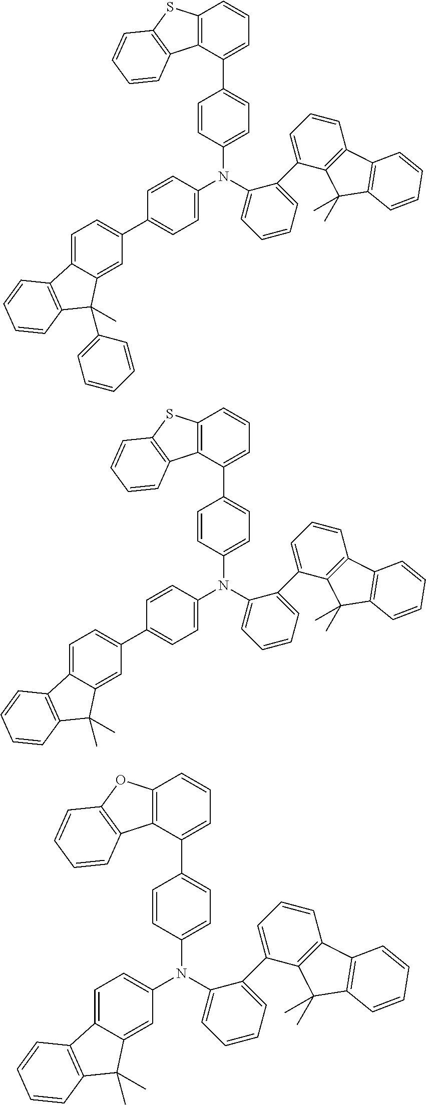

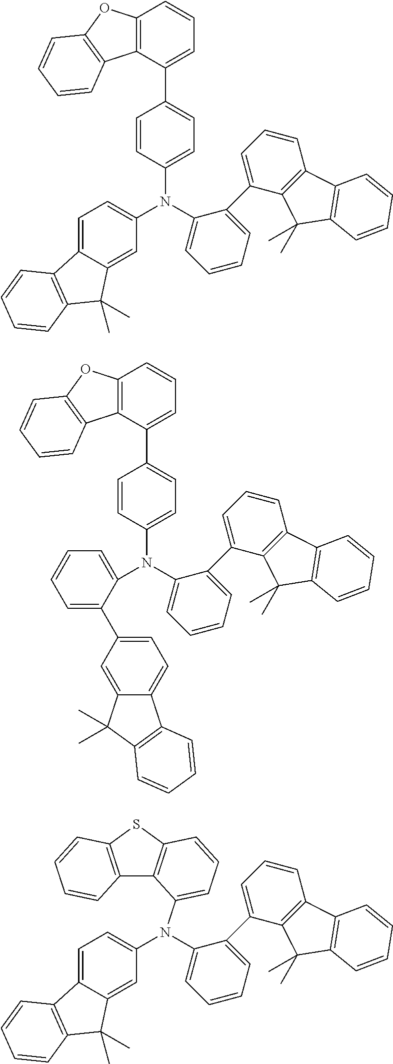

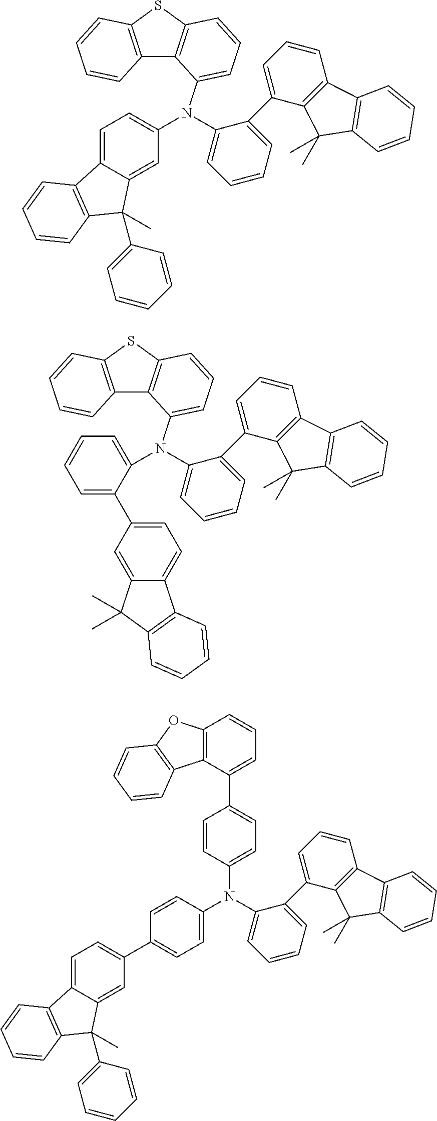

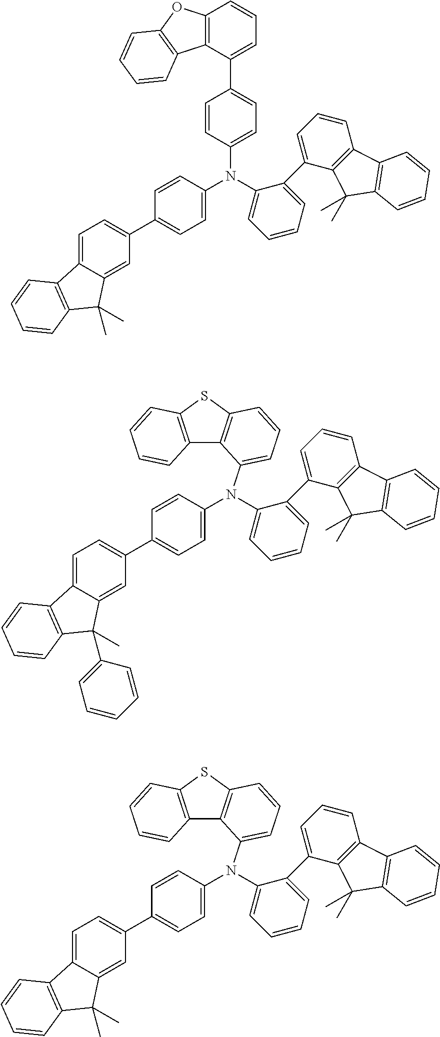 [SMILES: CC1(C)c2ccccc2-c2ccc(-c3ccc(N(c4ccc(-c5cccc6oc7ccccc7c56)cc4)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3cccc4sc5ccccc5c34)c21 ; CC1(C)c2ccccc2-c2ccc(-c3ccc(N(c4ccccc4-c4cccc5c4C(C)(C)c4ccccc4-5)c4cccc5sc6ccccc6c45)cc3)cc21]

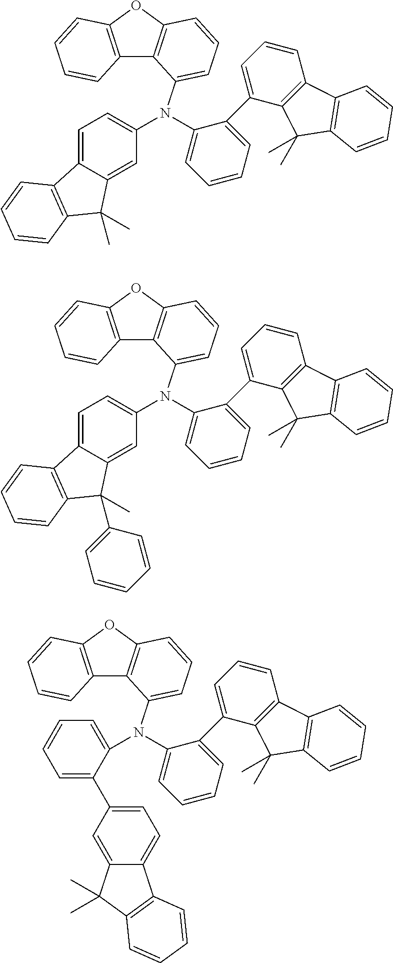

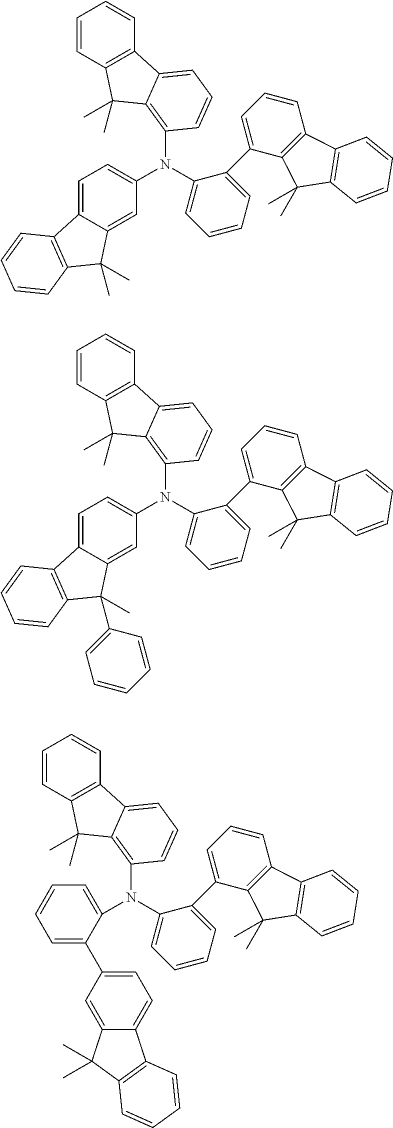

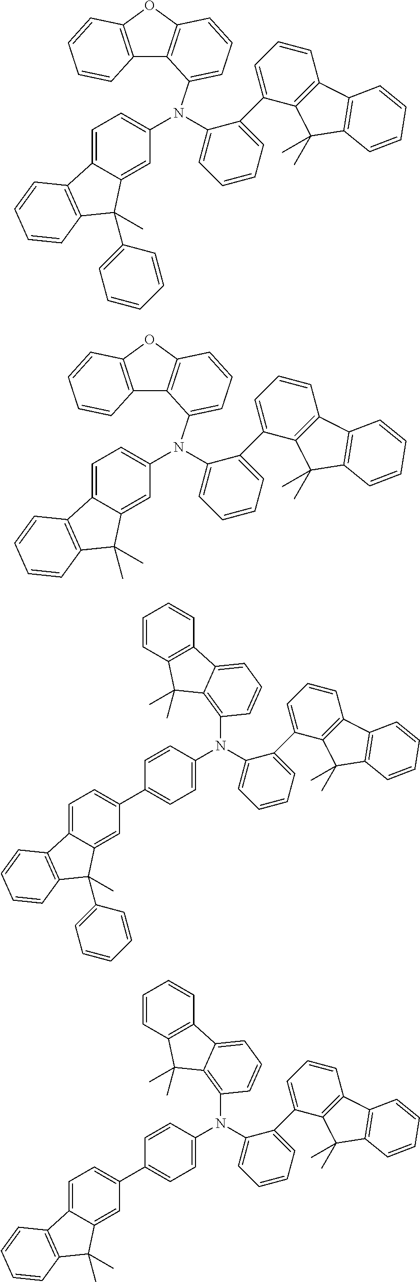

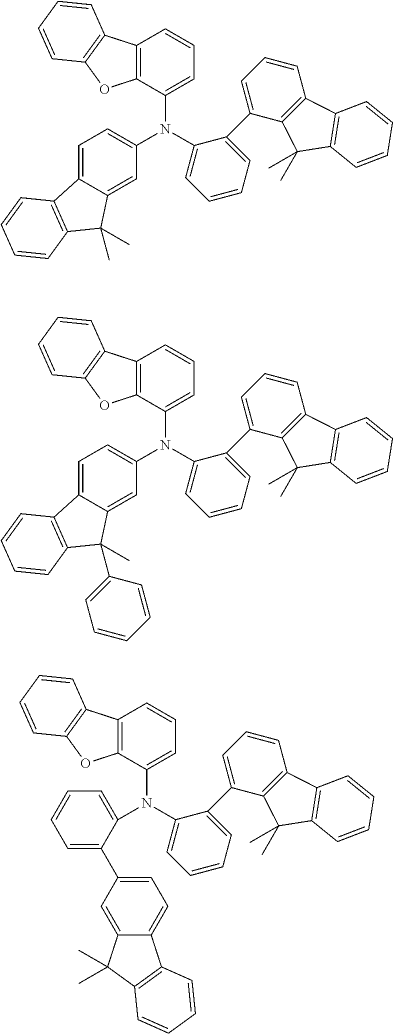 [SMILES: CC1(C)c2ccccc2-c2ccc(N(c3ccccc3-c3cccc4c3C(C)(C)c3ccccc3-4)c3cccc4c3oc3ccccc34)cc21 ; CC1(C)c2ccccc2-c2cccc(-c3ccccc3N(c3ccc4c(c3)C(C)(c3ccccc3)c3ccccc3-4)c3cccc4c3oc3ccccc34)c21 ; CC1(C)c2ccccc2-c2ccc(-c3ccccc3N(c3ccccc3-c3cccc4c3C(C)(C)c3ccccc3-4)c3cccc4c3oc3ccccc34)cc21]

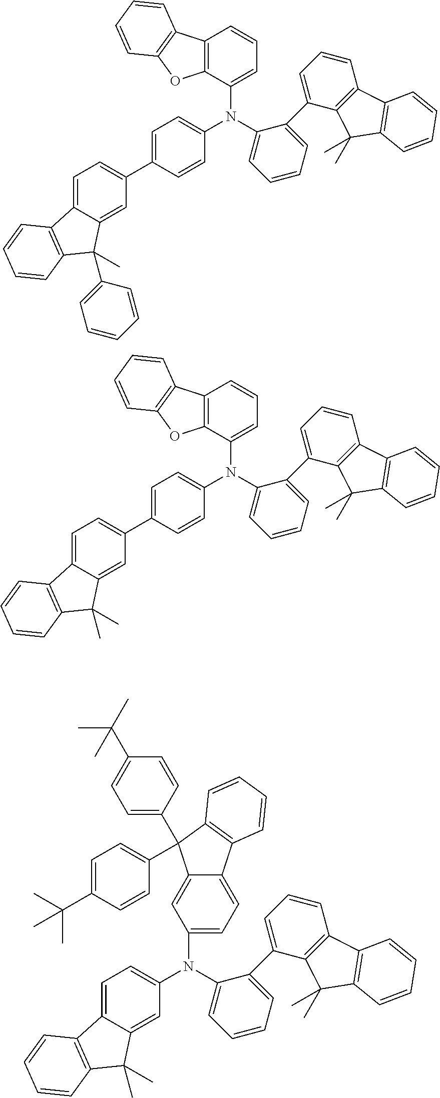

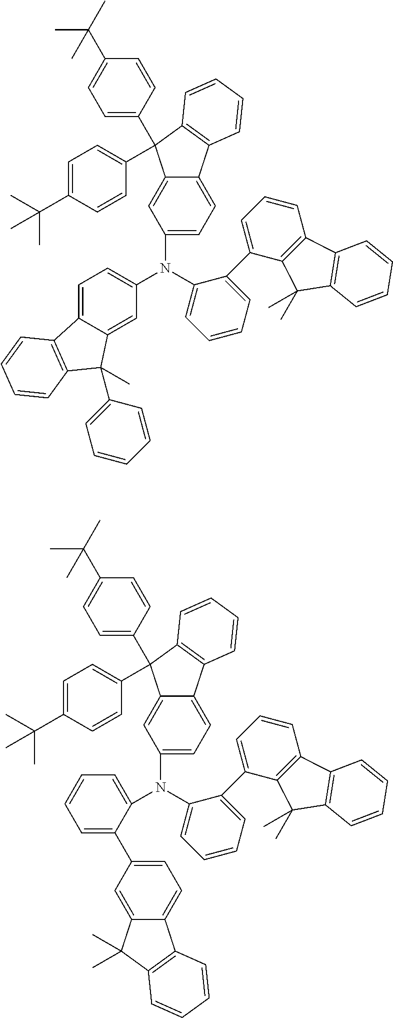 [SMILES: CC(C)(C)c1ccc(C2(c3ccc(C(C)(C)C)cc3)c3ccccc3-c3ccc(N(c4ccc5c(c4)C(C)(c4ccccc4)c4ccccc4-5)c4ccccc4-c4cccc5c4C(C)(C)c4ccccc4-5)cc32)cc1 ; CC(C)(C)c1ccc(C2(c3ccc(C(C)(C)C)cc3)c3ccccc3-c3ccc(N(c4ccccc4-c4ccc5c(c4)C(C)(C)c4ccccc4-5)c4ccccc4-c4cccc5c4C(C)(C)c4ccccc4-5)cc32)cc1]

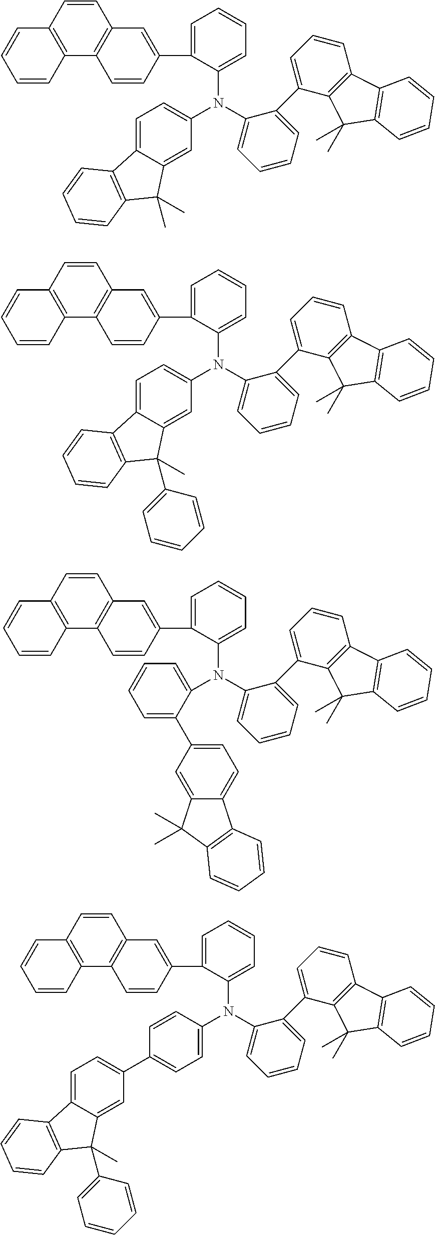

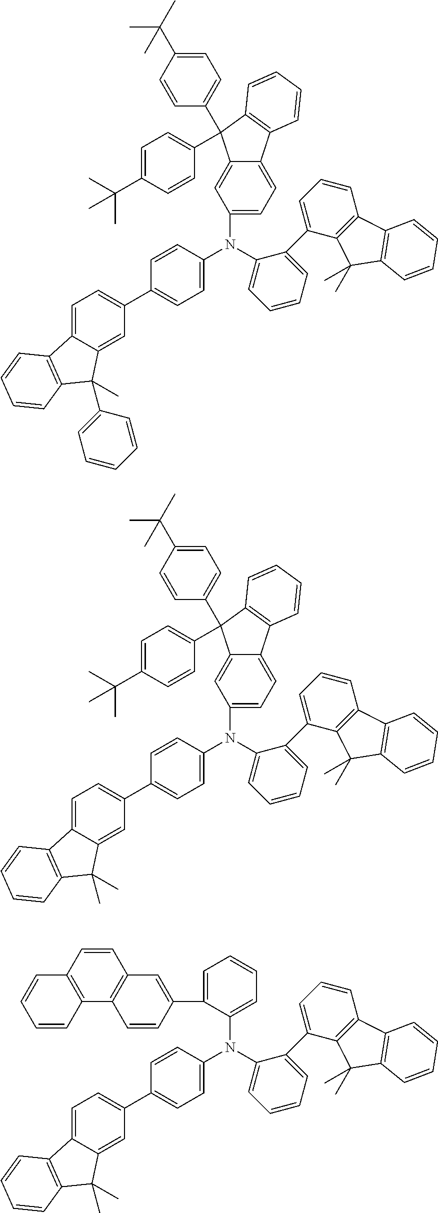 [SMILES: CC(C)(C)c1ccc(C2(c3ccc(C(C)(C)C)cc3)c3ccccc3-c3ccc(N(c4ccc(-c5ccc6c(c5)C(C)(c5ccccc5)c5ccccc5-6)cc4)c4ccccc4-c4cccc5c4C(C)(C)c4ccccc4-5)cc32)cc1 ; CC(C)(C)c1ccc(C2(c3ccc(C(C)(C)C)cc3)c3ccccc3-c3ccc(N(c4ccc(-c5ccc6c(c5)C(C)(C)c5ccccc5-6)cc4)c4ccccc4-c4cccc5c4C(C)(C)c4ccccc4-5)cc32)cc1 ; CC1(C)c2ccccc2-c2ccc(-c3ccc(N(c4ccccc4-c4ccc5c(ccc6ccccc65)c4)c4ccccc4-c4cccc5c4C(C)(C)c4ccccc4-5)cc3)cc21]

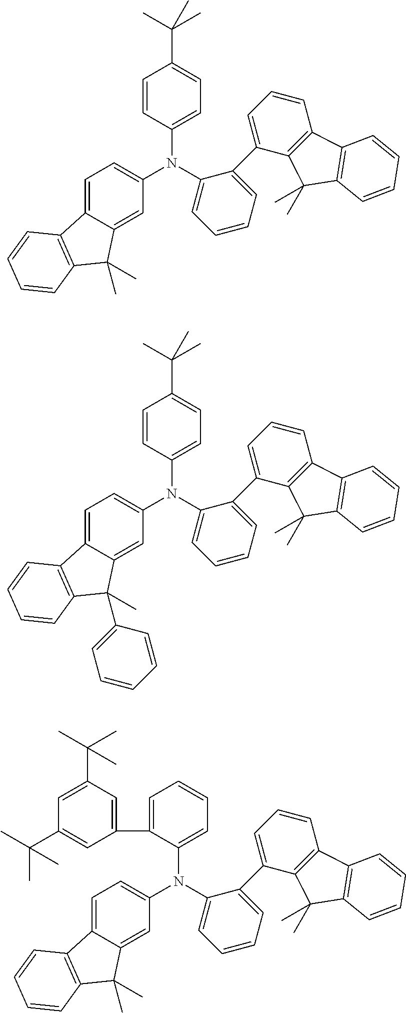

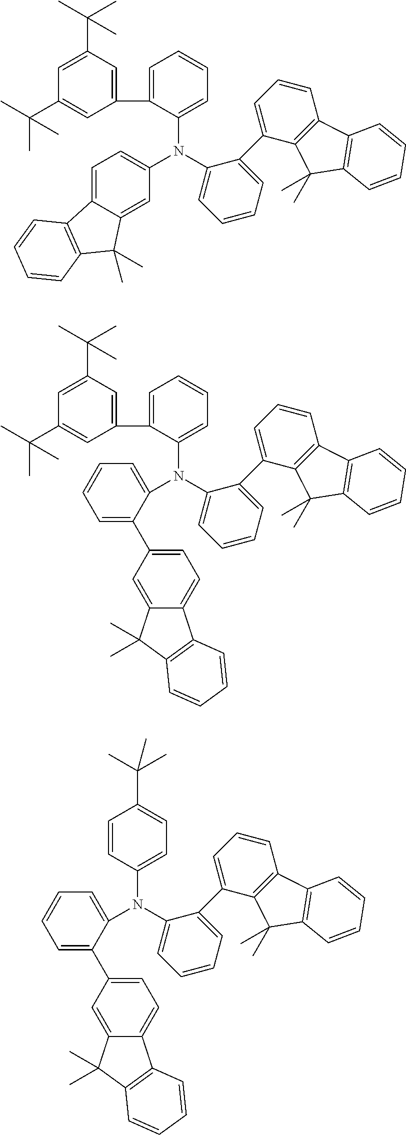

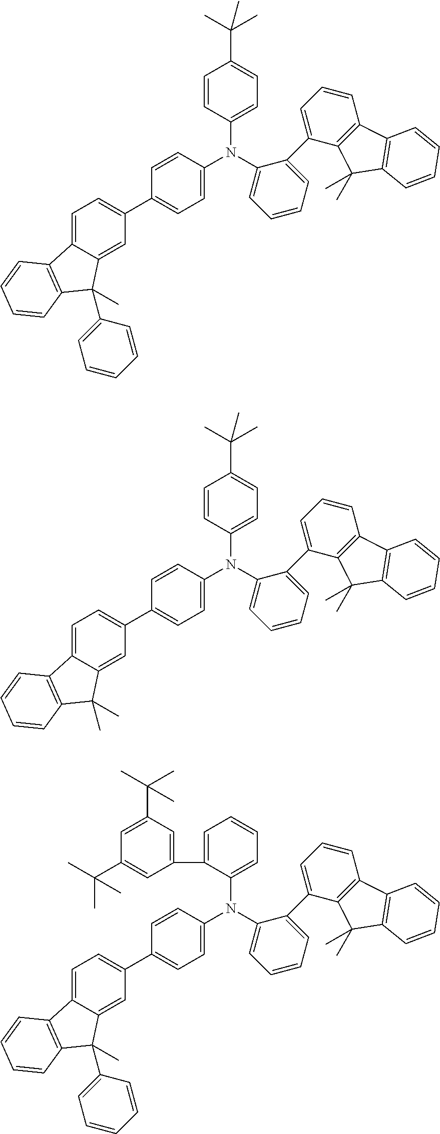 [SMILES: CC(C)(C)c1ccc(N(c2ccc(-c3ccc4c(c3)C(C)(c3ccccc3)c3ccccc3-4)cc2)c2ccccc2-c2cccc3c2C(C)(C)c2ccccc2-3)cc1 ; CC(C)(C)c1ccc(N(c2ccc(-c3ccc4c(c3)C(C)(C)c3ccccc3-4)cc2)c2ccccc2-c2cccc3c2C(C)(C)c2ccccc2-3)cc1 ; CC(C)(C)c1cc(-c2ccccc2N(c2ccc(-c3ccc4c(c3)C(C)(c3ccccc3)c3ccccc3-4)cc2)c2ccccc2-c2cccc3c2C(C)(C)c2ccccc2-3)cc(C(C)(C)C)c1]

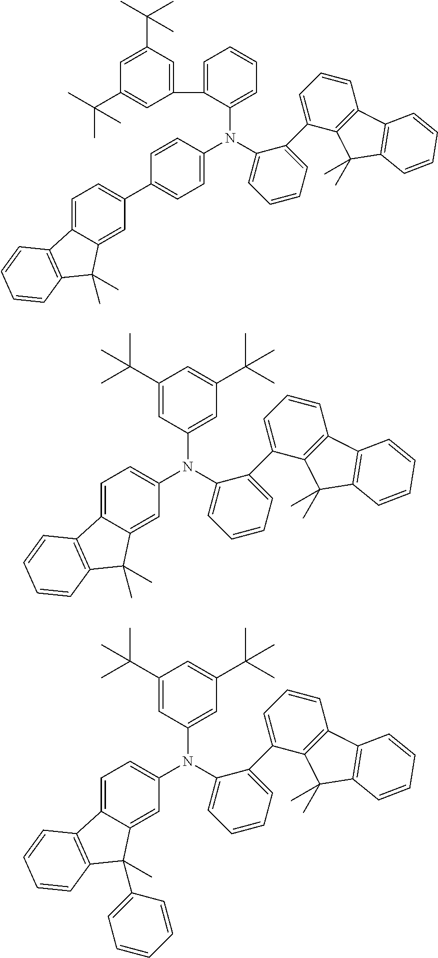

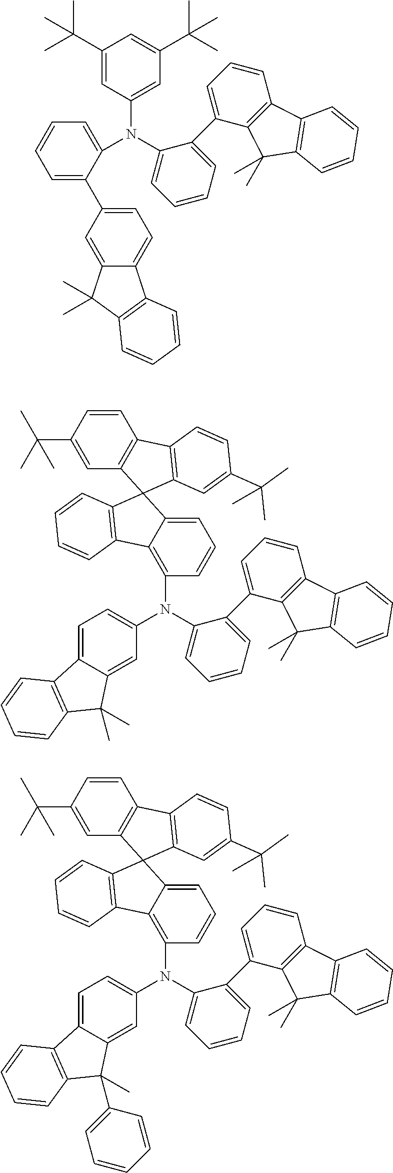

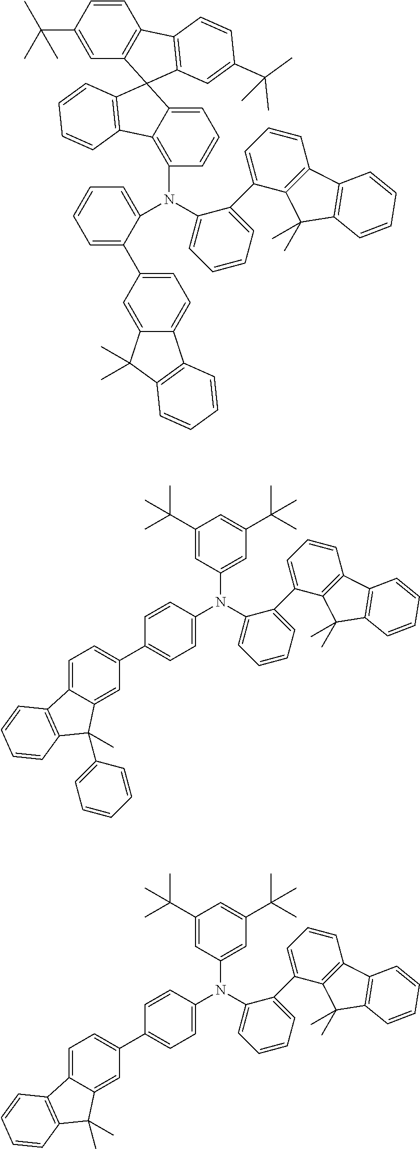 [SMILES: CC(C)(C)c1ccc2c(c1)C1(c3cc(C(C)(C)C)ccc3-2)c2ccccc2-c2c(N(c3ccccc3-c3ccc4c(c3)C(C)(C)c3ccccc3-4)c3ccccc3-c3cccc4c3C(C)(C)c3ccccc3-4)cccc21 ; CC(C)(C)c1cc(N(c2ccc(-c3ccc4c(c3)C(C)(c3ccccc3)c3ccccc3-4)cc2)c2ccccc2-c2cccc3c2C(C)(C)c2ccccc2-3)cc(C(C)(C)C)c1 ; CC(C)(C)c1cc(N(c2ccc(-c3ccc4c(c3)C(C)(C)c3ccccc3-4)cc2)c2ccccc2-c2cccc3c2C(C)(C)c2ccccc2-3)cc(C(C)(C)C)c1]

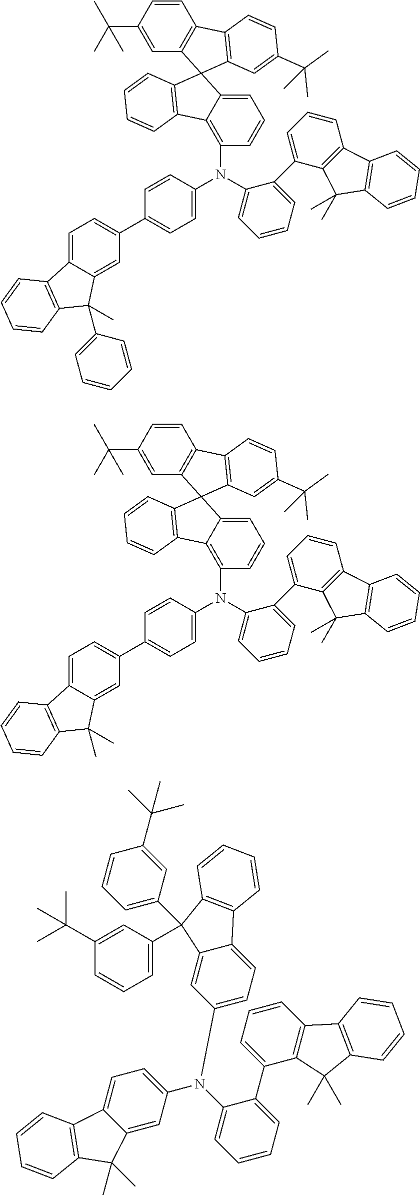

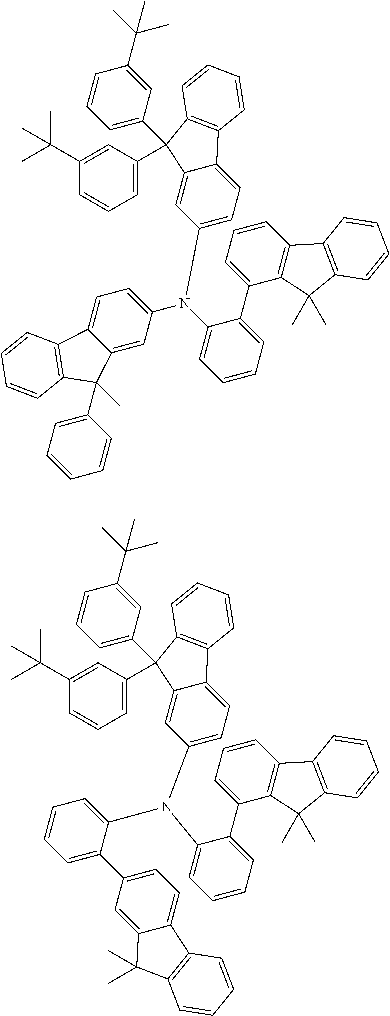 [SMILES: CC(C)(C)c1cccc(C2(c3cccc(C(C)(C)C)c3)c3ccccc3-c3ccc(N(c4ccc5c(c4)C(C)(c4ccccc4)c4ccccc4-5)c4ccccc4-c4cccc5c4C(C)(C)c4ccccc4-5)cc32)c1 ; CC(C)(C)c1cccc(C2(c3cccc(C(C)(C)C)c3)c3ccccc3-c3ccc(N(c4ccccc4-c4ccc5c(c4)C(C)(C)c4ccccc4-5)c4ccccc4-c4cccc5c4C(C)(C)c4ccccc4-5)cc32)c1]

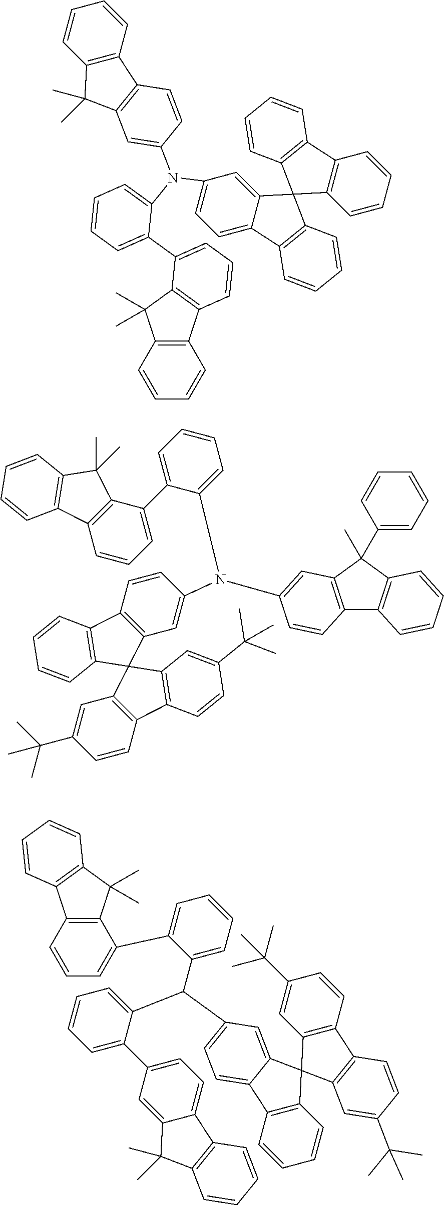

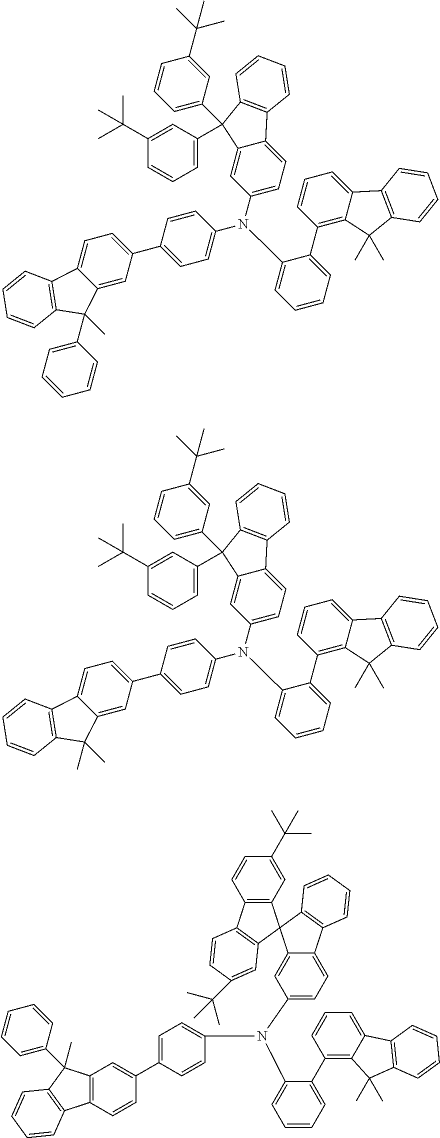 [SMILES: CC(C)(C)c1cccc(C2(c3cccc(C(C)(C)C)c3)c3ccccc3-c3ccc(N(c4ccc(-c5ccc6c(c5)C(C)(c5ccccc5)c5ccccc5-6)cc4)c4ccccc4-c4cccc5c4C(C)(C)c4ccccc4-5)cc32)c1 ; CC(C)(C)c1cccc(C2(c3cccc(C(C)(C)C)c3)c3ccccc3-c3ccc(N(c4ccc(-c5ccc6c(c5)C(C)(C)c5ccccc5-6)cc4)c4ccccc4-c4cccc5c4C(C)(C)c4ccccc4-5)cc32)c1 ; CC(C)(C)c1ccc2c(c1)C1(c3ccccc3-c3ccc(N(c4ccc(-c5ccc6c(c5)C(C)(c5ccccc5)c5ccccc5-6)cc4)c4ccccc4-c4cccc5c4C(C)(C)c4ccccc4-5)cc31)c1cc(C(C)(C)C)ccc1-2]

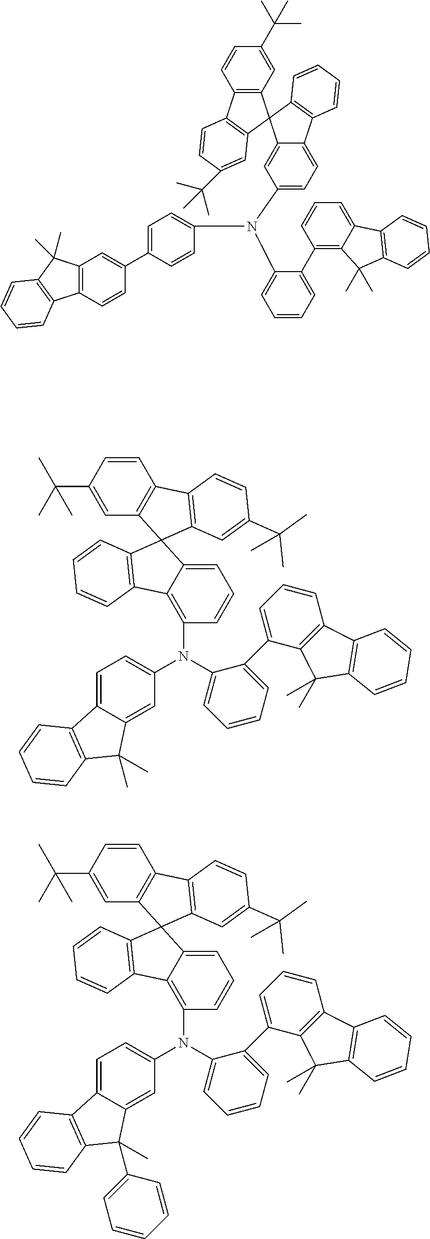 [SMILES: CC(C)(C)c1ccc2c(c1)C1(c3ccccc3-c3ccc(N(c4ccc(-c5ccc6c(c5)C(C)(C)c5ccccc5-6)cc4)c4ccccc4-c4cccc5c4C(C)(C)c4ccccc4-5)cc31)c1cc(C(C)(C)C)ccc1-2 ; CC(C)(C)c1ccc2c(c1)C1(c3cc(C(C)(C)C)ccc3-2)c2ccccc2-c2c(N(c3ccc4c(c3)C(C)(C)c3ccccc3-4)c3ccccc3-c3cccc4c3C(C)(C)c3ccccc3-4)cccc21 ; CC(C)(C)c1ccc2c(c1)C1(c3cc(C(C)(C)C)ccc3-2)c2ccccc2-c2c(N(c3ccc4c(c3)C(C)(c3ccccc3)c3ccccc3-4)c3ccccc3-c3cccc4c3C(C)(C)c3ccccc3-4)cccc21]

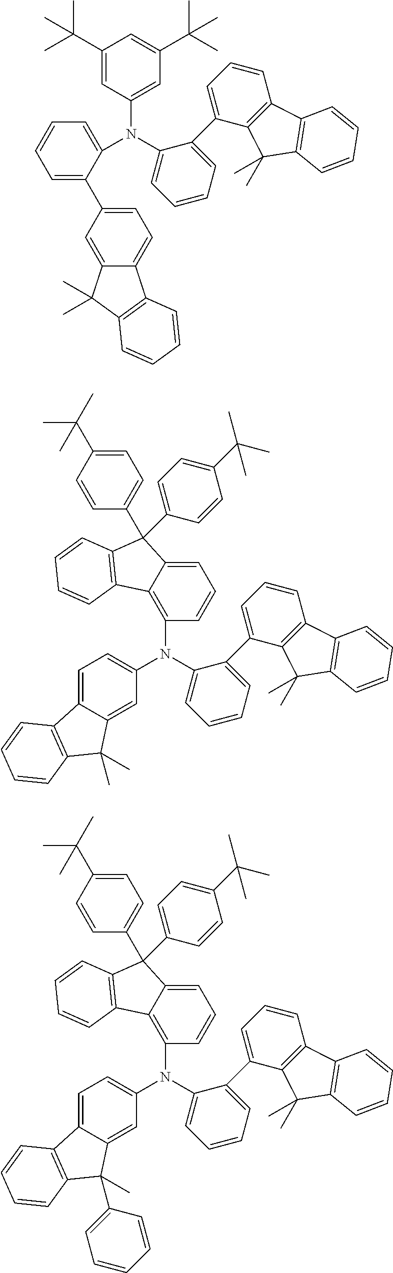

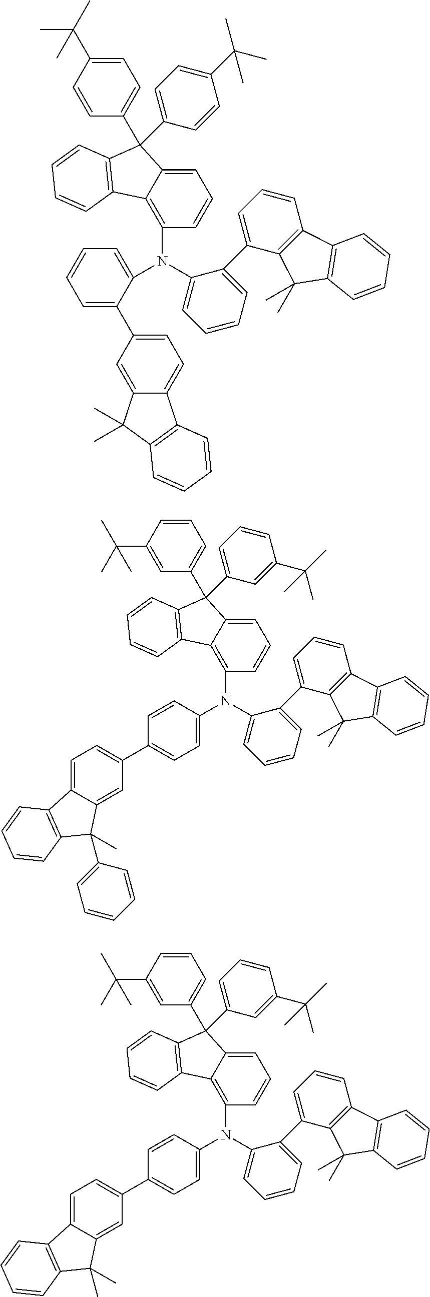

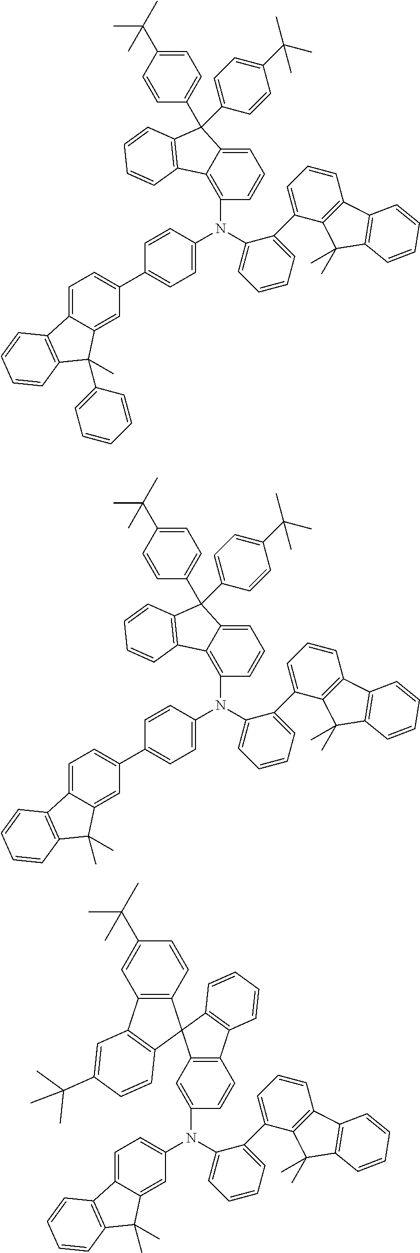

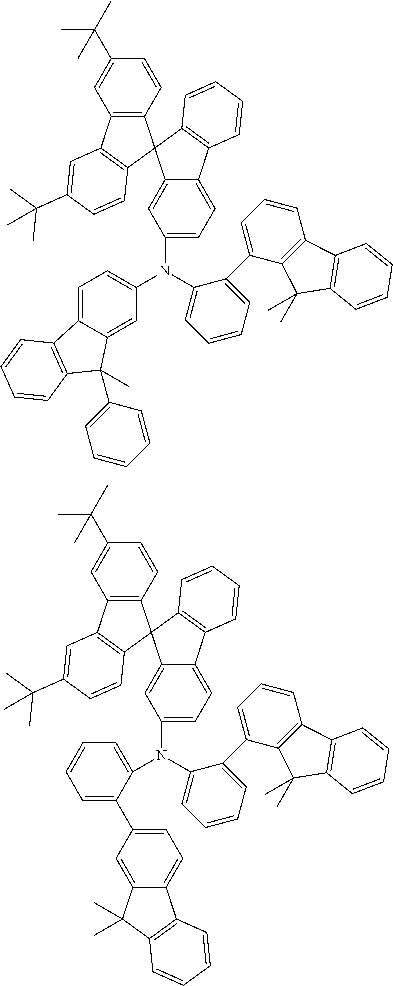

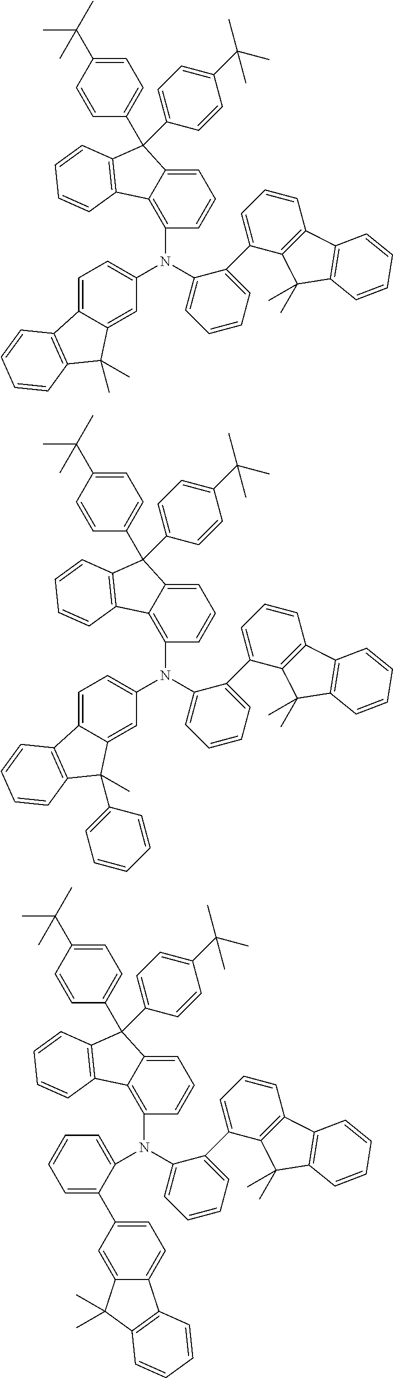

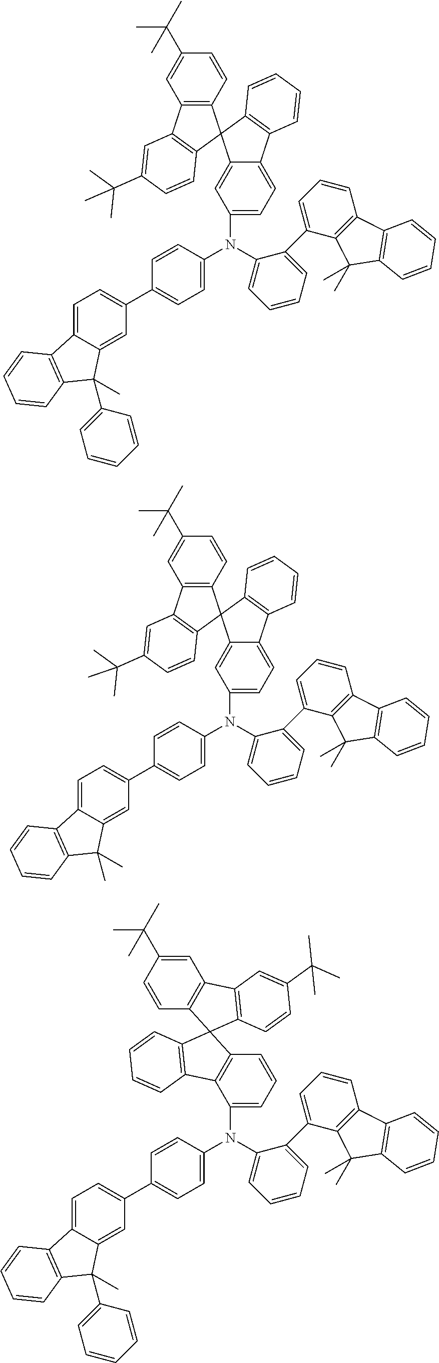 [SMILES: CC(C)(C)c1ccc2c(c1)-c1cc(C(C)(C)C)ccc1C21c2ccccc2-c2ccc(N(c3ccc(-c4ccc5c(c4)C(C)(c4ccccc4)c4ccccc4-5)cc3)c3ccccc3-c3cccc4c3C(C)(C)c3ccccc3-4)cc21 ; CC(C)(C)c1ccc2c(c1)-c1cc(C(C)(C)C)ccc1C21c2ccccc2-c2ccc(N(c3ccc(-c4ccc5c(c4)C(C)(C)c4ccccc4-5)cc3)c3ccccc3-c3cccc4c3C(C)(C)c3ccccc3-4)cc21 ; CC(C)(C)c1ccc2c(c1)-c1cc(C(C)(C)C)ccc1C21c2ccccc2-c2c(N(c3ccc(-c4ccc5c(c4)C(C)(c4ccccc4)c4ccccc4-5)cc3)c3ccccc3-c3cccc4c3C(C)(C)c3ccccc3-4)cccc21]

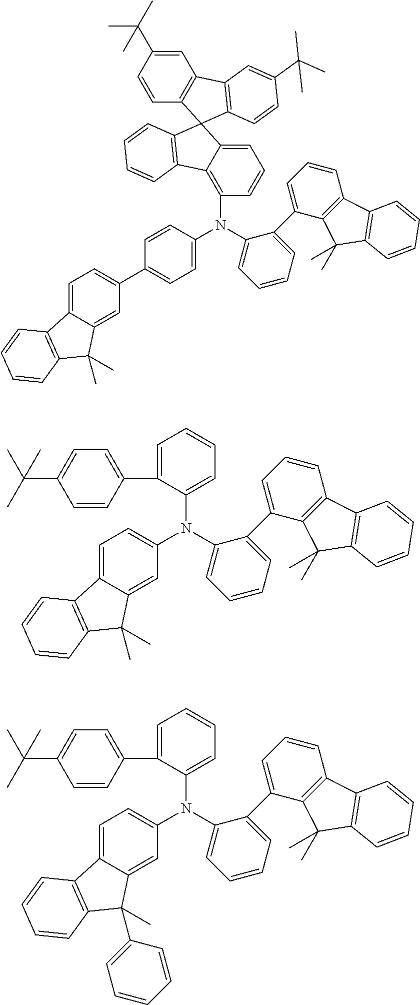 [SMILES: CC(C)(C)c1ccc2c(c1)-c1cc(C(C)(C)C)ccc1C21c2ccccc2-c2c(N(c3ccc(-c4ccc5c(c4)C(C)(C)c4ccccc4-5)cc3)c3ccccc3-c3cccc4c3C(C)(C)c3ccccc3-4)cccc21 ; CC(C)(C)c1ccc(-c2ccccc2N(c2ccc3c(c2)C(C)(C)c2ccccc2-3)c2ccccc2-c2cccc3c2C(C)(C)c2ccccc2-3)cc1 ; CC(C)(C)c1ccc(-c2ccccc2N(c2ccc3c(c2)C(C)(c2ccccc2)c2ccccc2-3)c2ccccc2-c2cccc3c2C(C)(C)c2ccccc2-3)cc1]

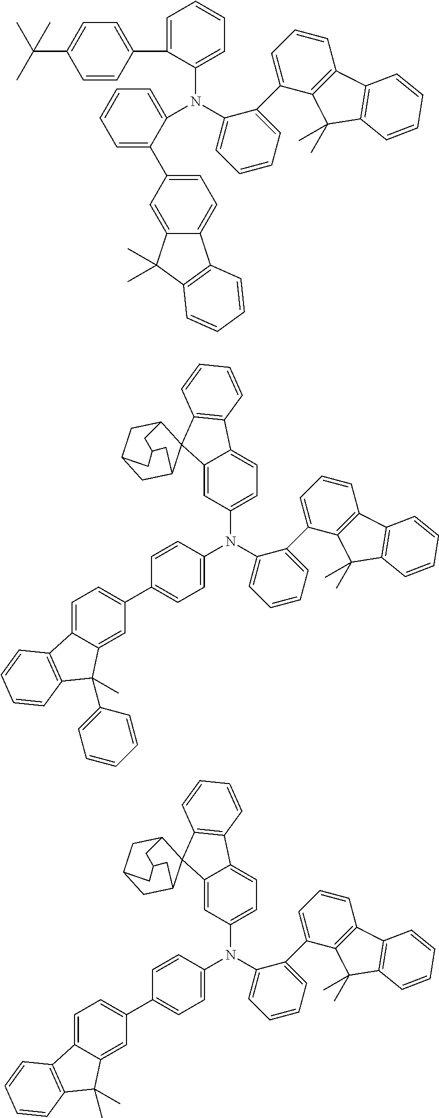

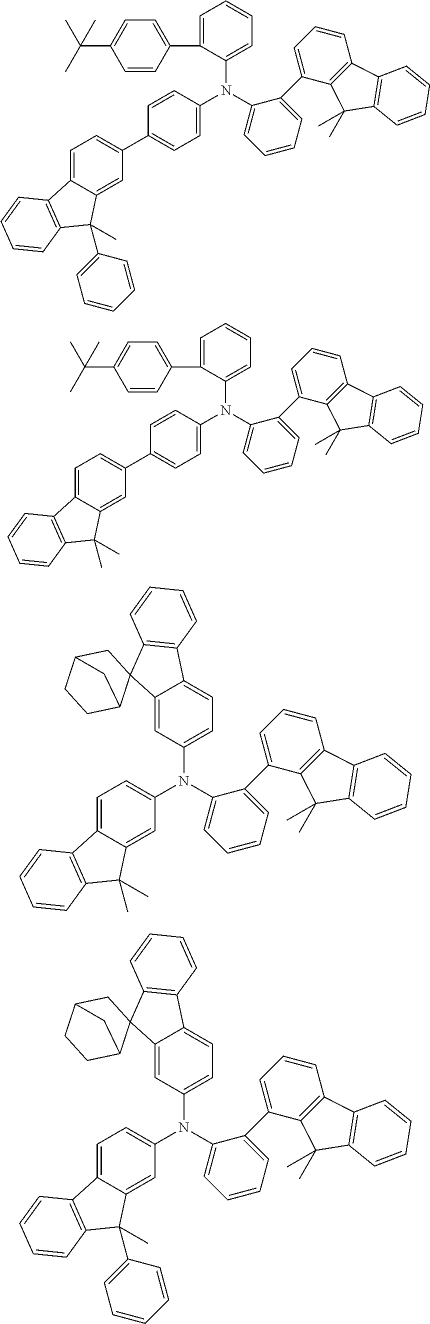

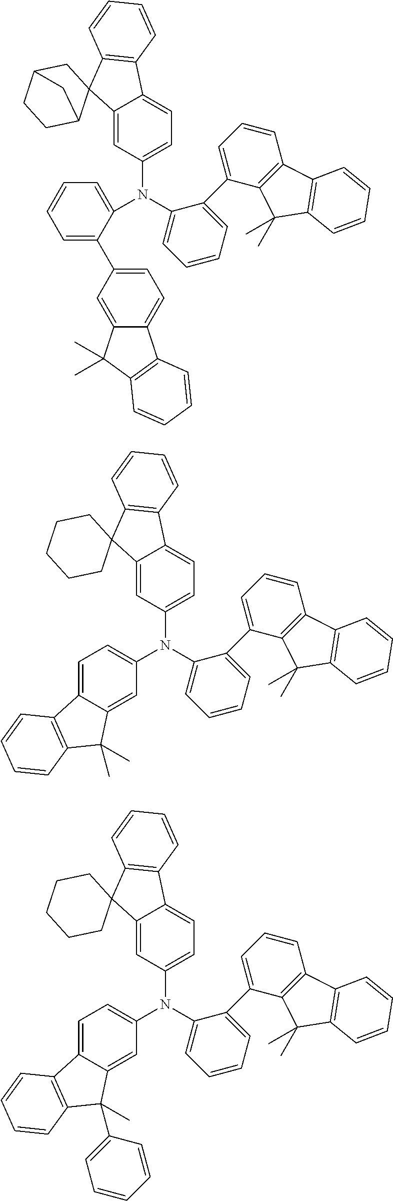

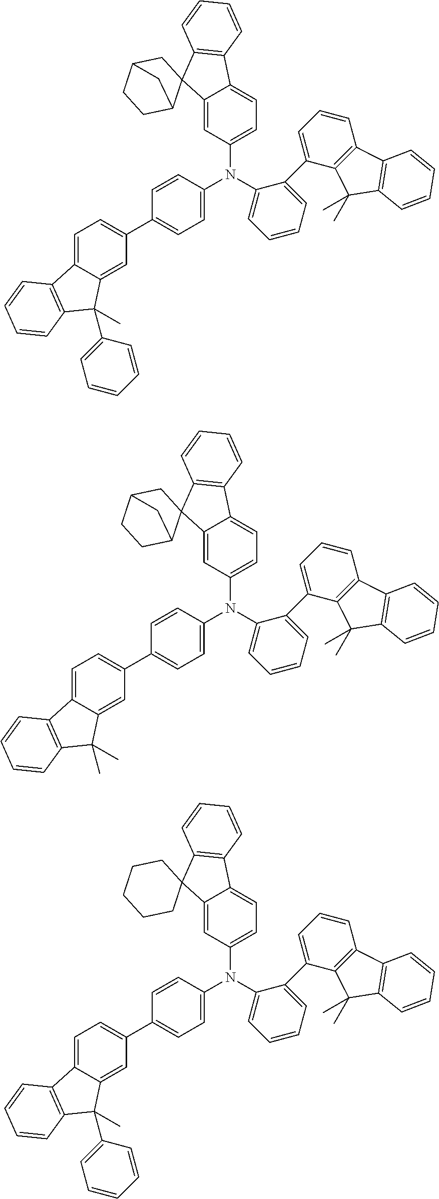 [SMILES: CC1(C)c2ccccc2-c2cccc(-c3ccccc3N(c3ccc(-c4ccc5c(c4)C(C)(c4ccccc4)c4ccccc4-5)cc3)c3ccc4c(c3)C3(CC5CCC3C5)c3ccccc3-4)c21 ; CC1(C)c2ccccc2-c2ccc(-c3ccc(N(c4ccc5c(c4)C4(CC6CCC4C6)c4ccccc4-5)c4ccccc4-c4cccc5c4C(C)(C)c4ccccc4-5)cc3)cc21 ; CC1(C)c2ccccc2-c2cccc(-c3ccccc3N(c3ccc(-c4ccc5c(c4)C(C)(c4ccccc4)c4ccccc4-5)cc3)c3ccc4c(c3)C3(CCCCC3)c3ccccc3-4)c21]

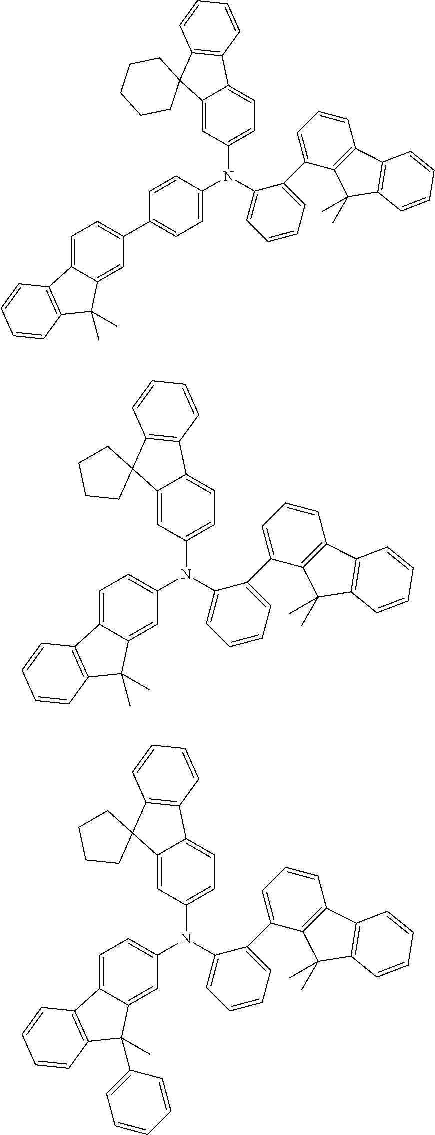

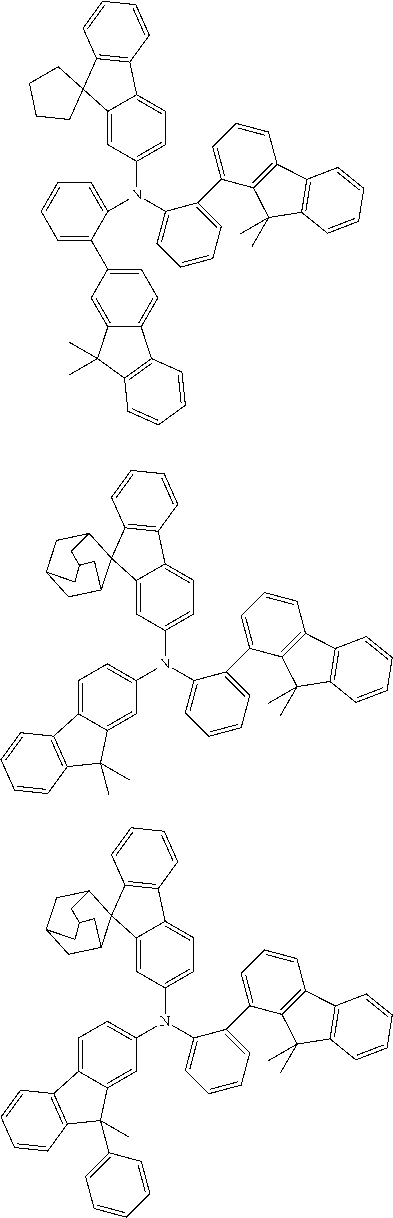

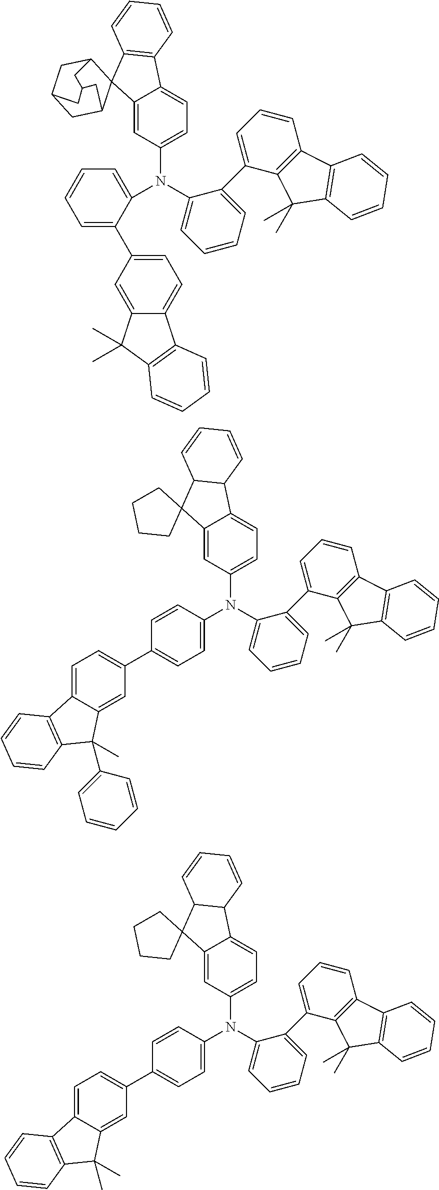

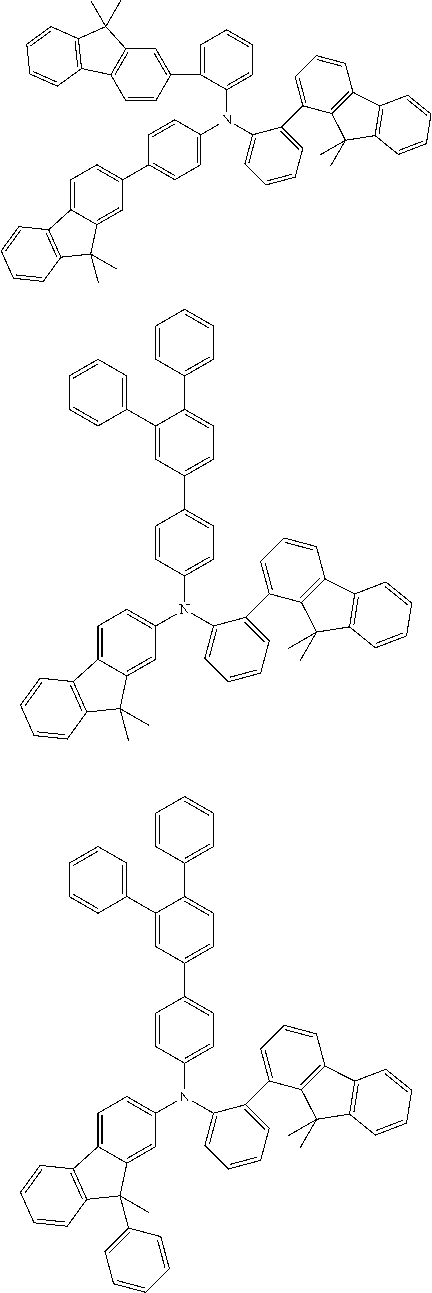 [SMILES: CC1(C)c2ccccc2-c2ccc(-c3ccc(N(c4ccccc4-c4ccc5c(c4)C(C)(C)c4ccccc4-5)c4ccccc4-c4cccc5c4C(C)(C)c4ccccc4-5)cc3)cc21 ; CC1(C)c2ccccc2-c2ccc(N(c3ccc(-c4ccc(-c5ccccc5)c(-c5ccccc5)c4)cc3)c3ccccc3-c3cccc4c3C(C)(C)c3ccccc3-4)cc21 ; CC1(C)c2ccccc2-c2cccc(-c3ccccc3N(c3ccc(-c4ccc(-c5ccccc5)c(-c5ccccc5)c4)cc3)c3ccc4c(c3)C(C)(c3ccccc3)c3ccccc3-4)c21]

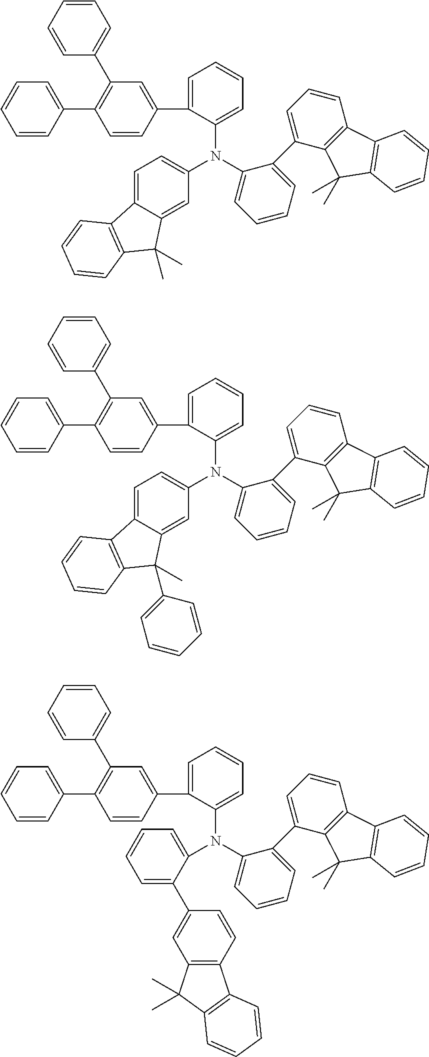

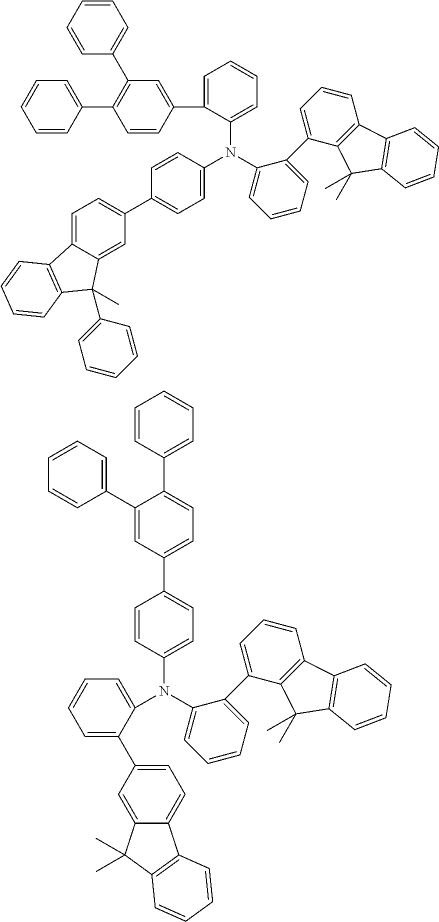

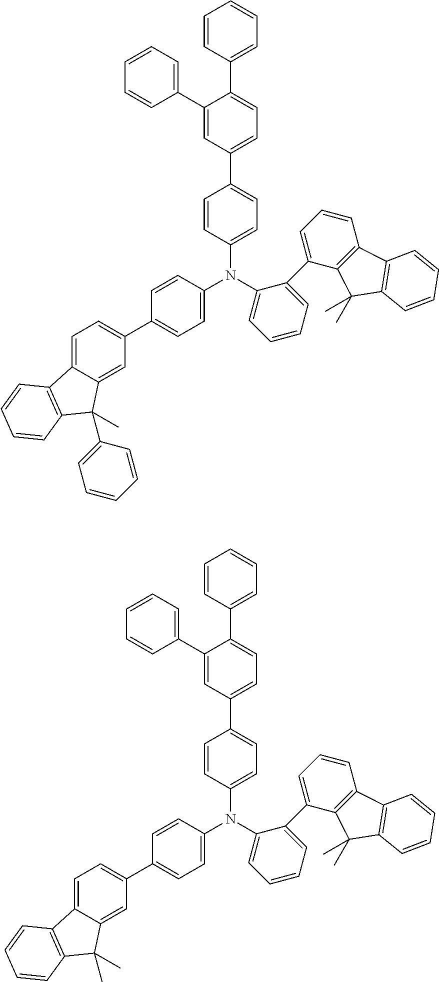 [SMILES: CC1(C)c2ccccc2-c2cccc(-c3ccccc3N(c3ccc(-c4ccc(-c5ccccc5)c(-c5ccccc5)c4)cc3)c3ccc(-c4ccc5c(c4)C(C)(c4ccccc4)c4ccccc4-5)cc3)c21 ; CC1(C)c2ccccc2-c2ccc(-c3ccc(N(c4ccc(-c5ccc(-c6ccccc6)c(-c6ccccc6)c5)cc4)c4ccccc4-c4cccc5c4C(C)(C)c4ccccc4-5)cc3)cc21]

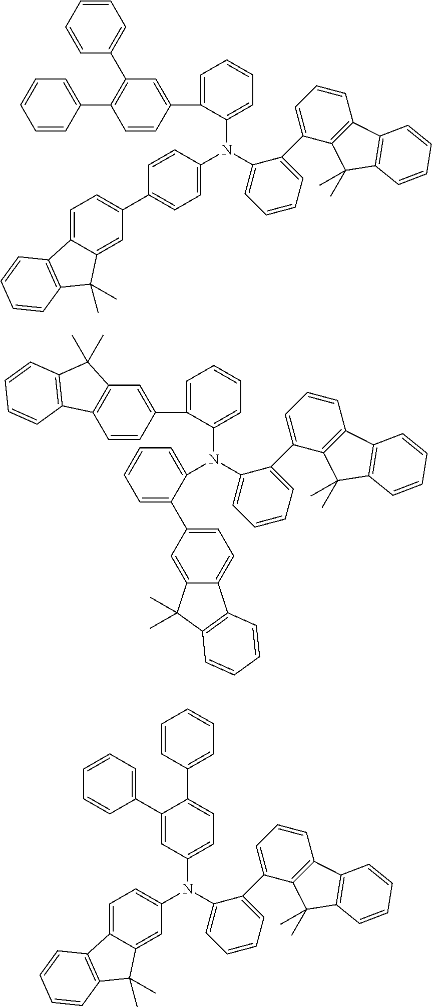

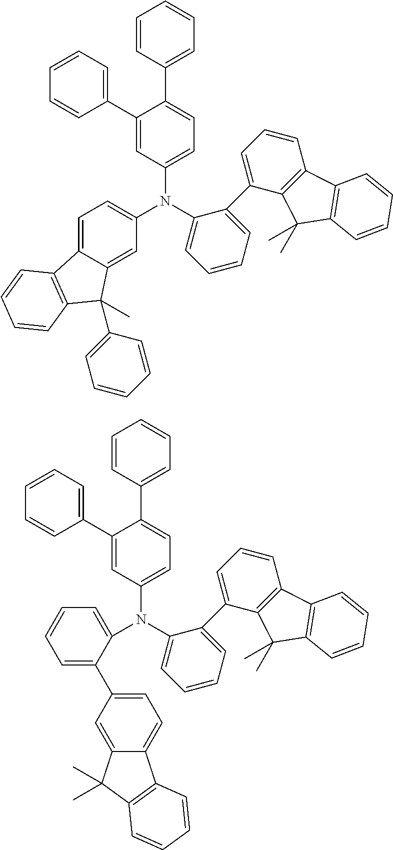

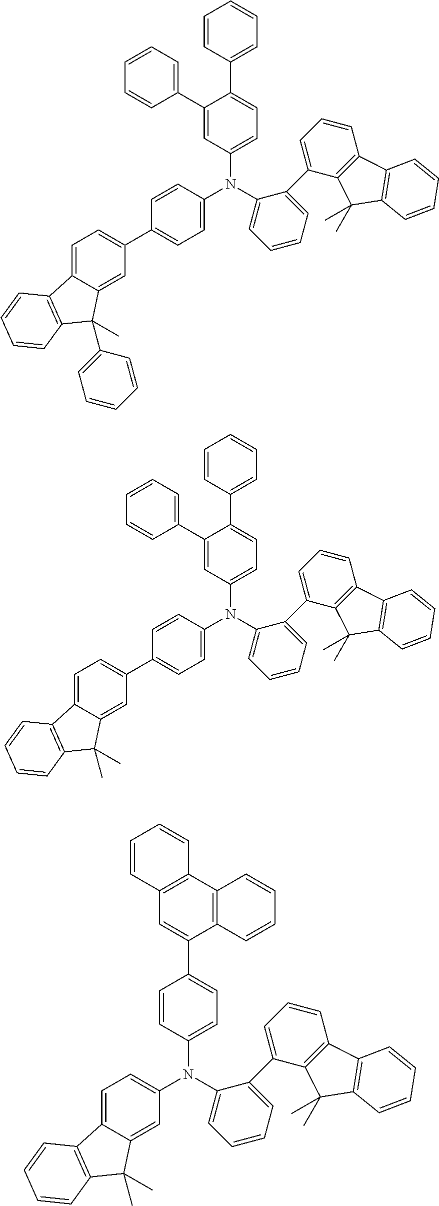 [SMILES: CC1(C)c2ccccc2-c2cccc(-c3ccccc3N(c3ccc(-c4ccc5c(c4)C(C)(c4ccccc4)c4ccccc4-5)cc3)c3ccc(-c4ccccc4)c(-c4ccccc4)c3)c21 ; CC1(C)c2ccccc2-c2ccc(-c3ccc(N(c4ccc(-c5ccccc5)c(-c5ccccc5)c4)c4ccccc4-c4cccc5c4C(C)(C)c4ccccc4-5)cc3)cc21 ; CC1(C)c2ccccc2-c2ccc(N(c3ccc(-c4cc5ccccc5c5ccccc45)cc3)c3ccccc3-c3cccc4c3C(C)(C)c3ccccc3-4)cc21]

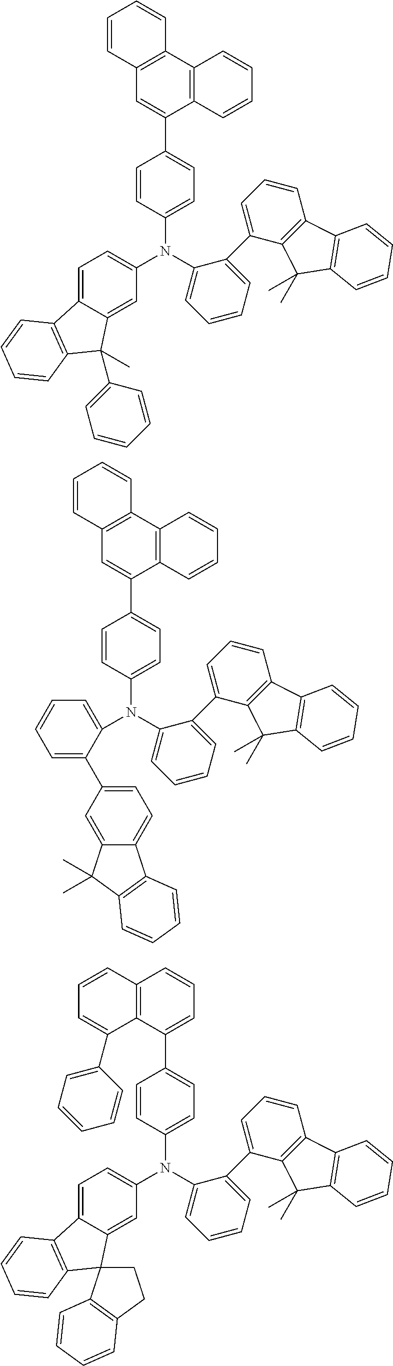

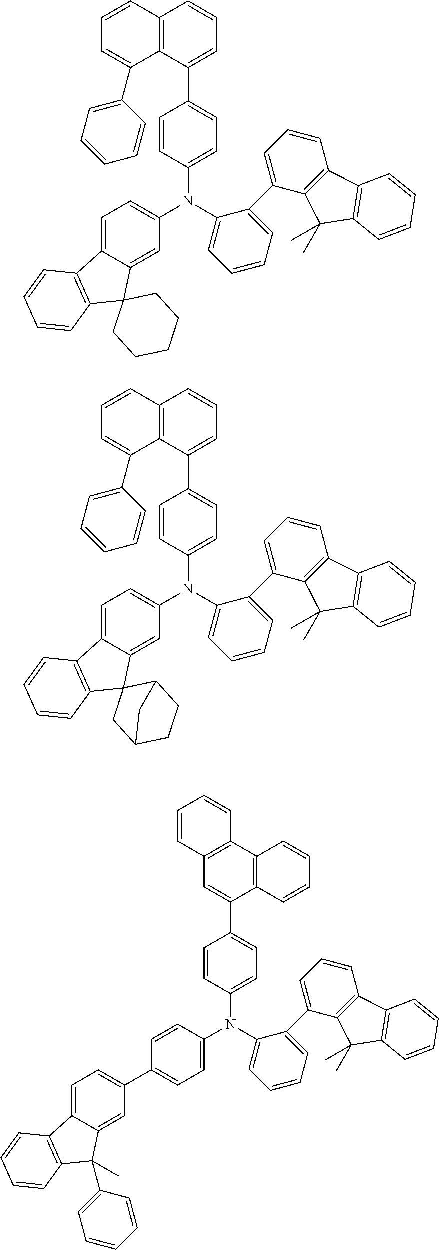

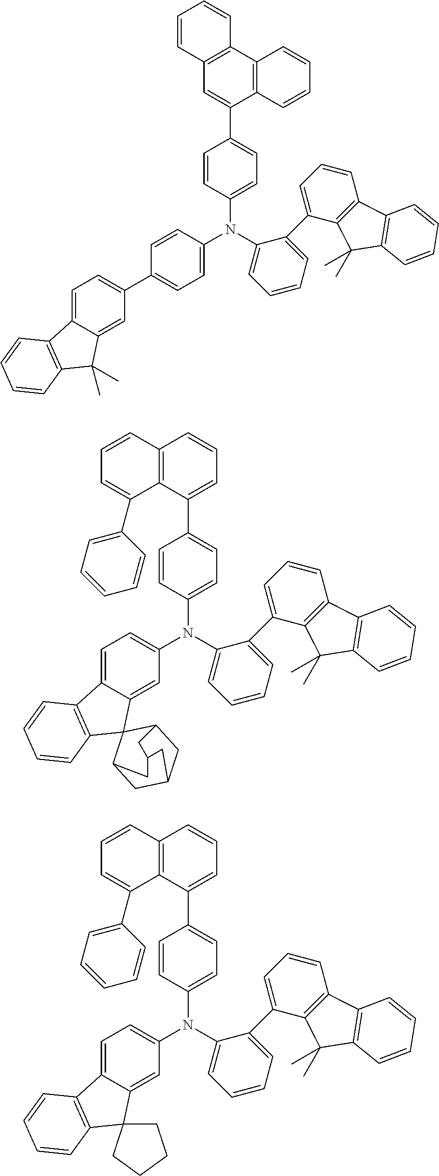

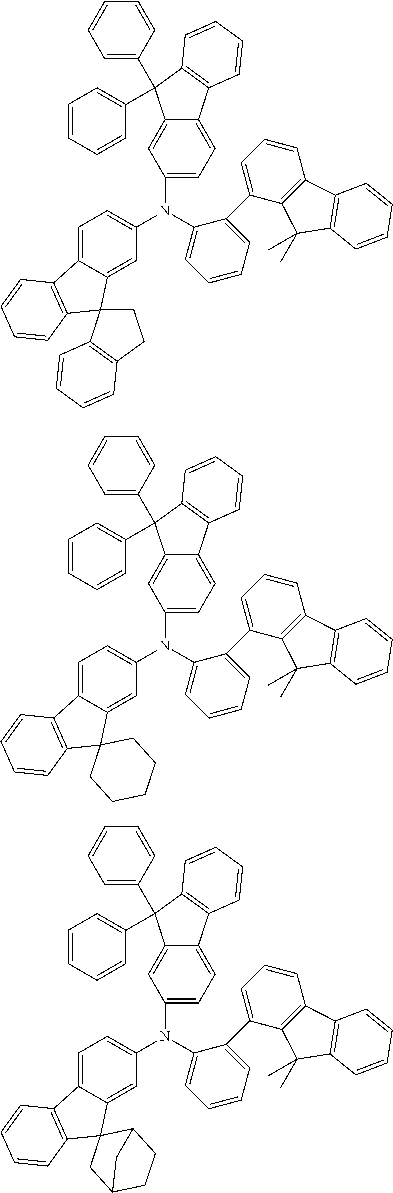

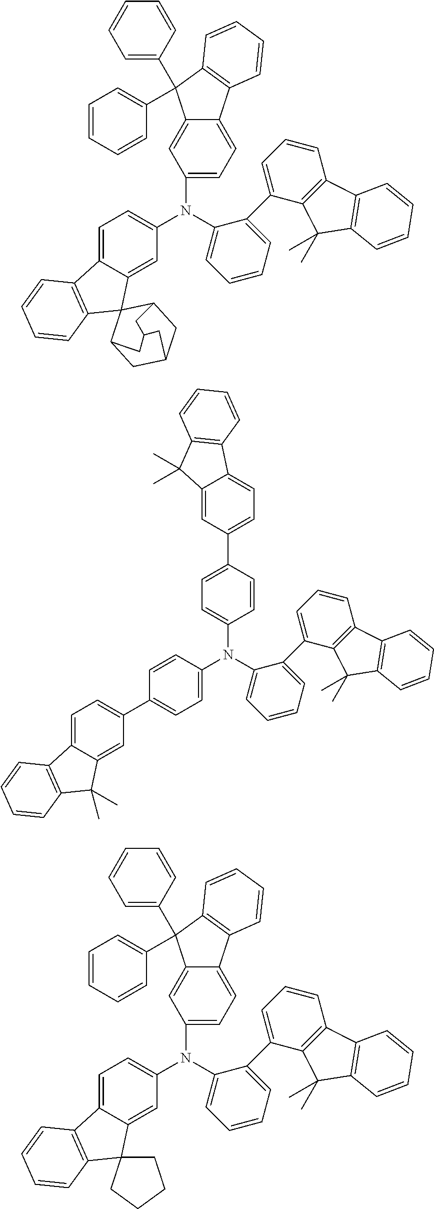

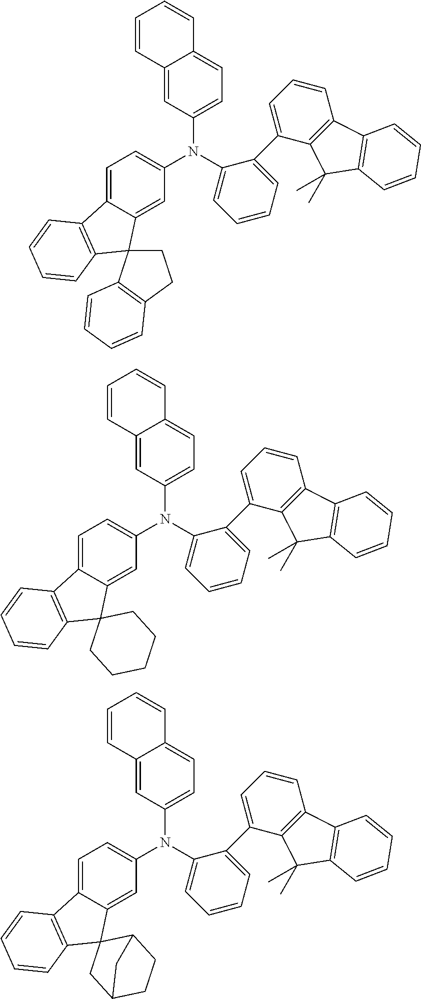

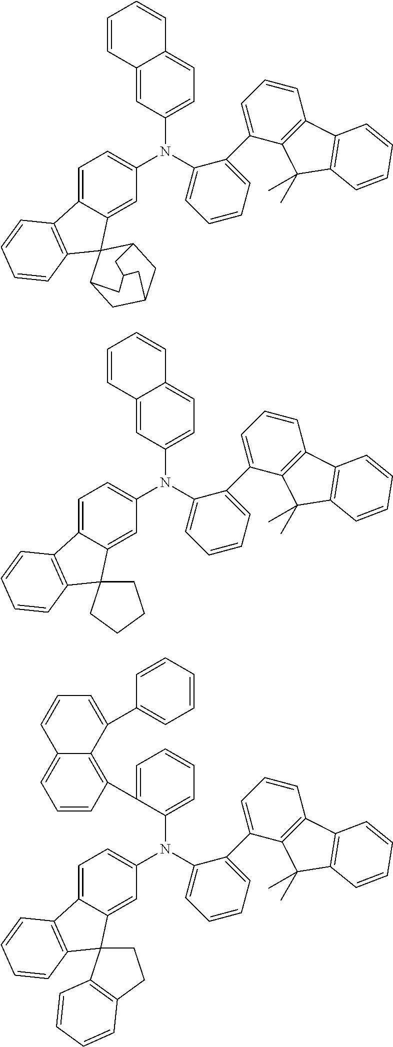

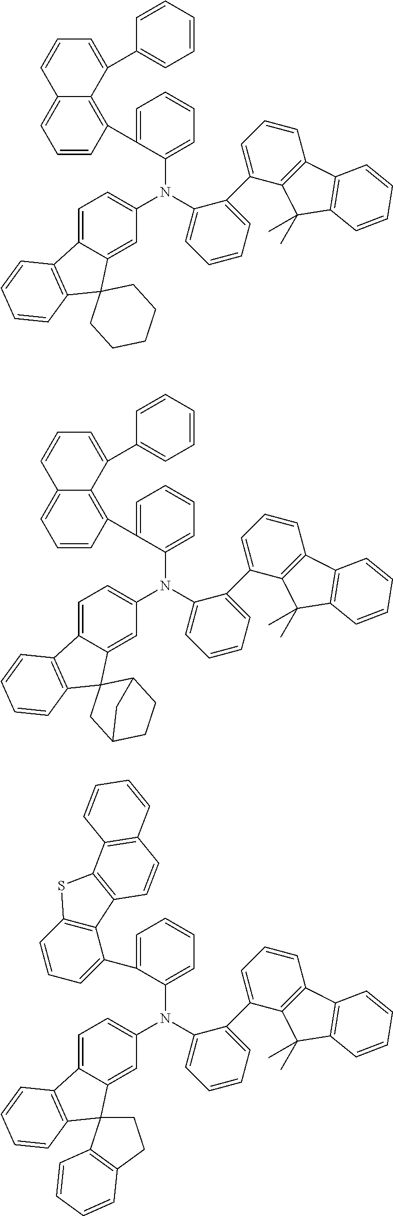

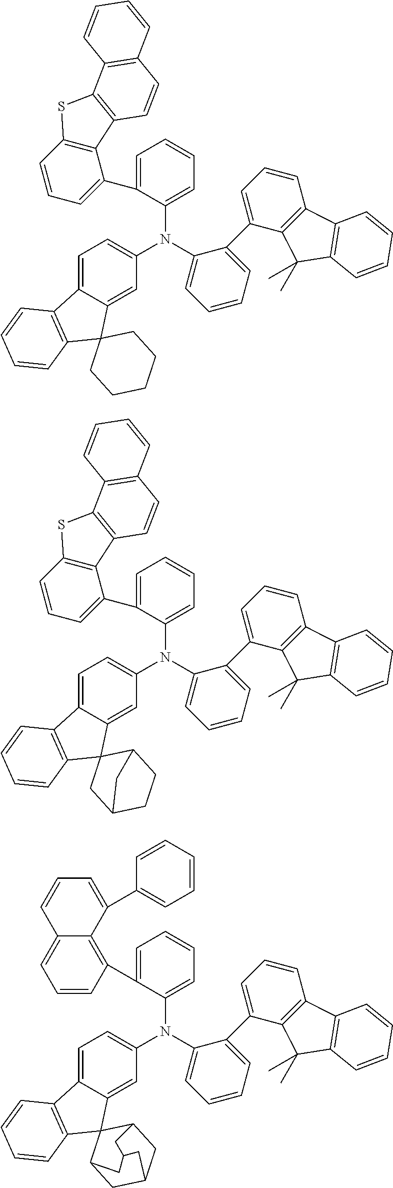

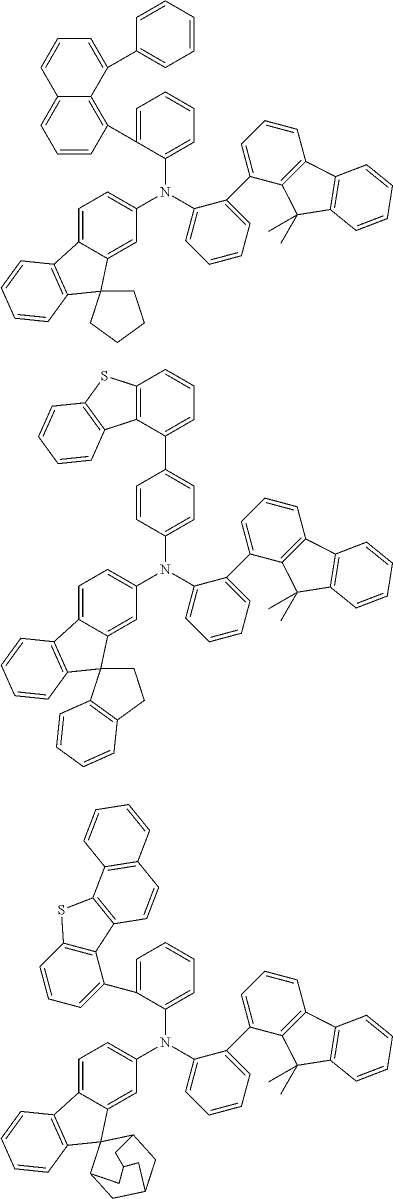

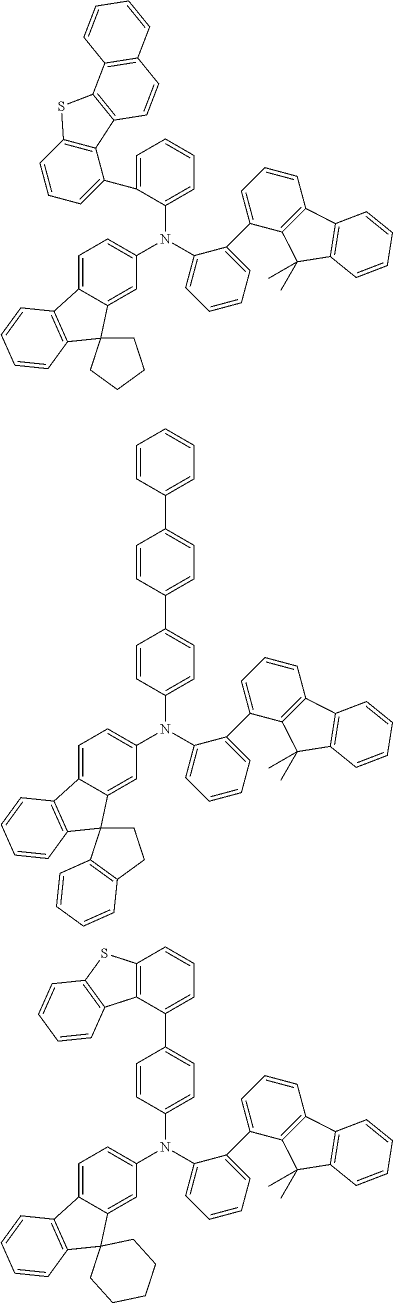

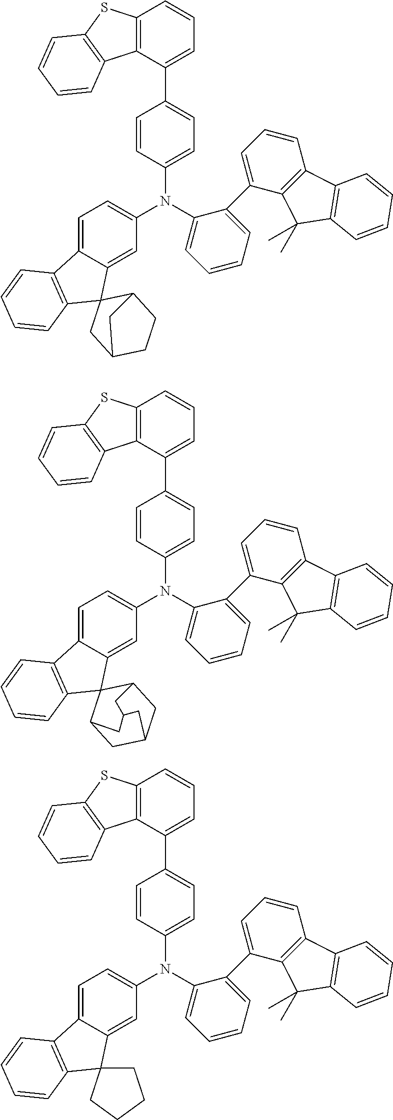

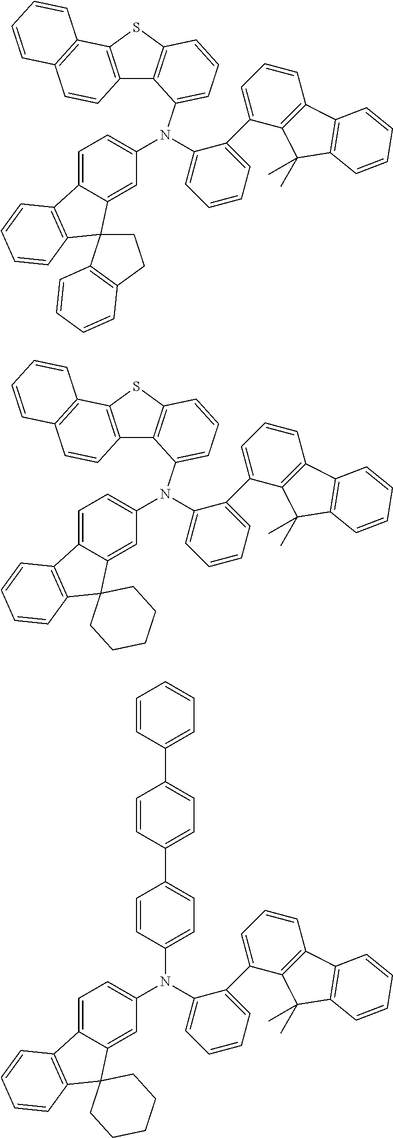

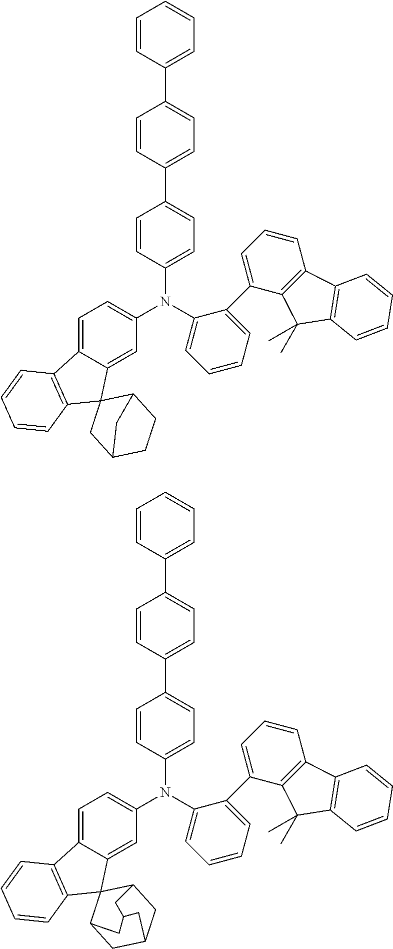

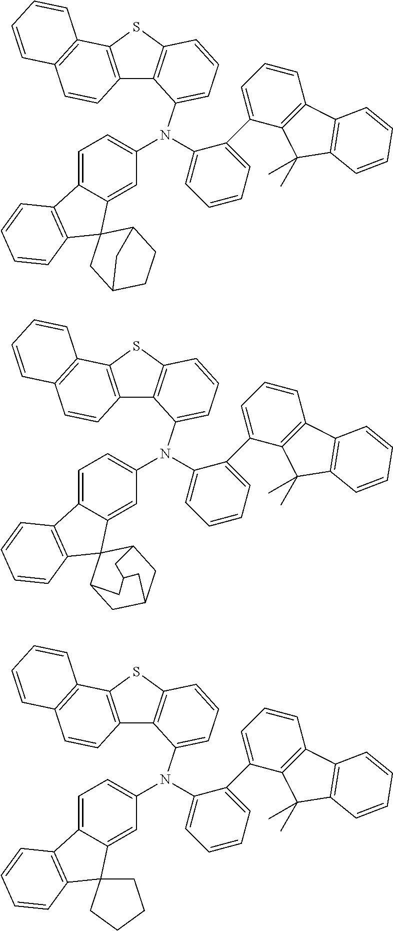 [SMILES: CC1(C)c2ccccc2-c2cccc(-c3ccccc3N(c3ccc4c(c3)C3(CC5CCC3C5)c3ccccc3-4)c3cccc4sc5c6ccccc6ccc5c34)c21 ; CC1(C)c2ccccc2-c2cccc(-c3ccccc3N(c3ccc4c(c3)C3(c5ccccc5-4)C4CC5CC(C4)CC3C5)c3cccc4sc5c6ccccc6ccc5c34)c21 ; CC1(C)c2ccccc2-c2cccc(-c3ccccc3N(c3ccc4c(c3)C3(CCCC3)c3ccccc3-4)c3cccc4sc5c6ccccc6ccc5c34)c21]

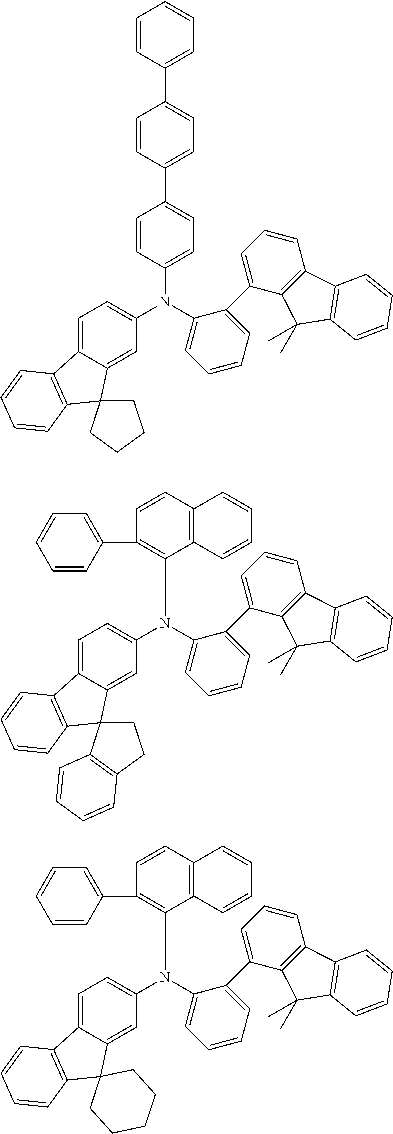

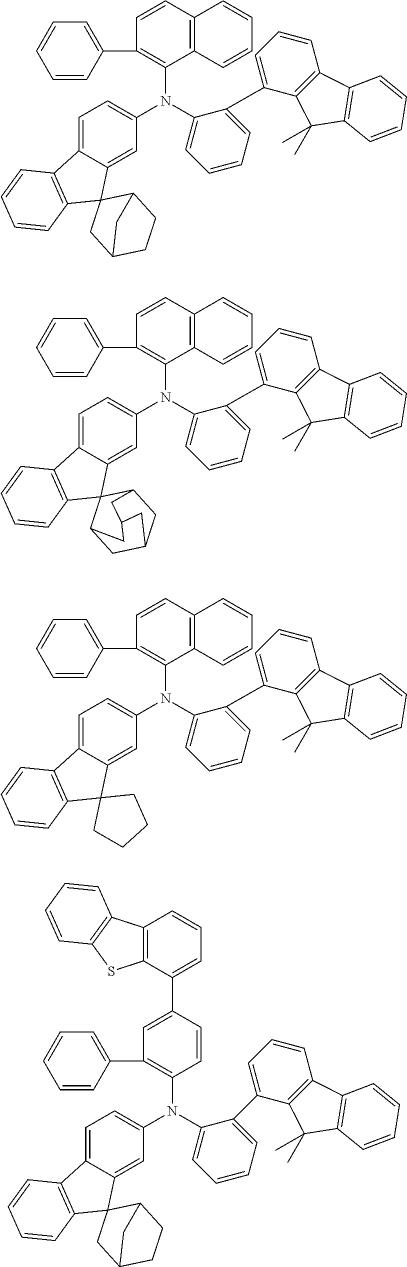

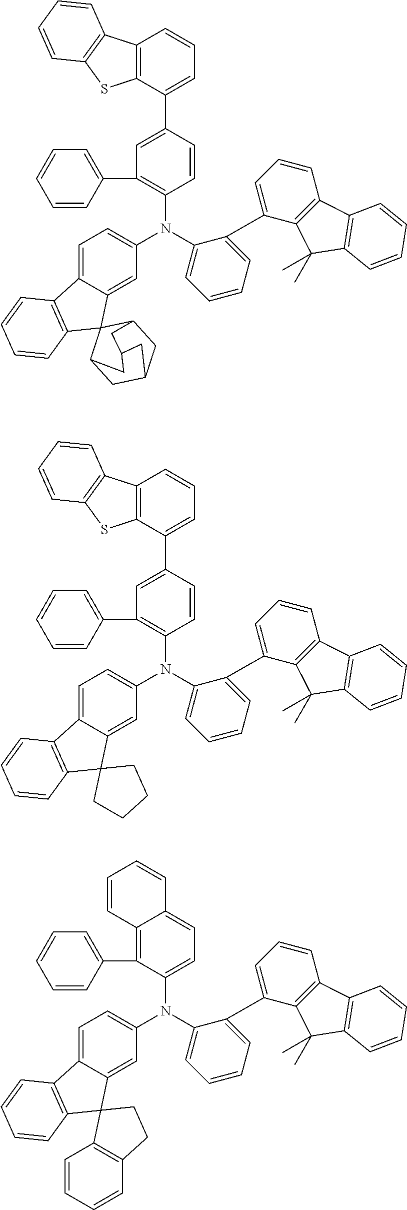

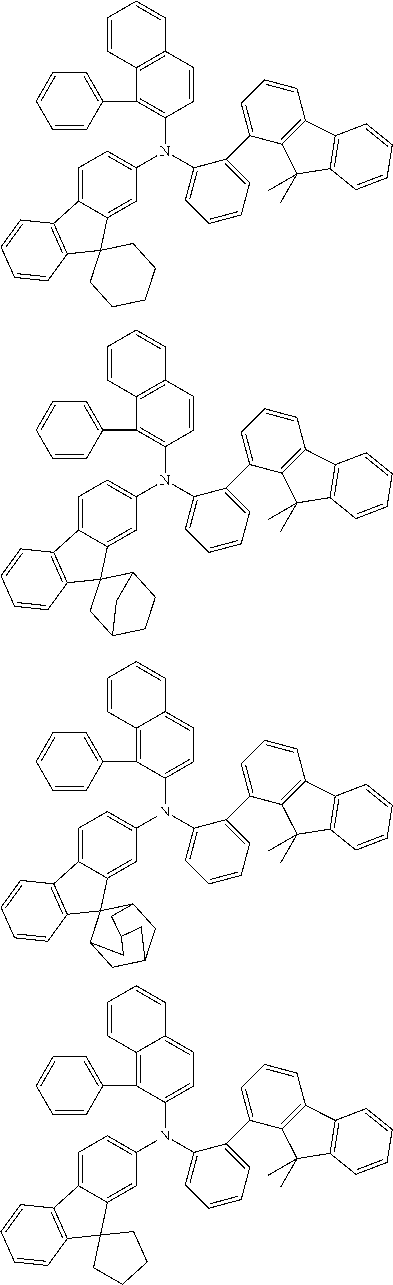

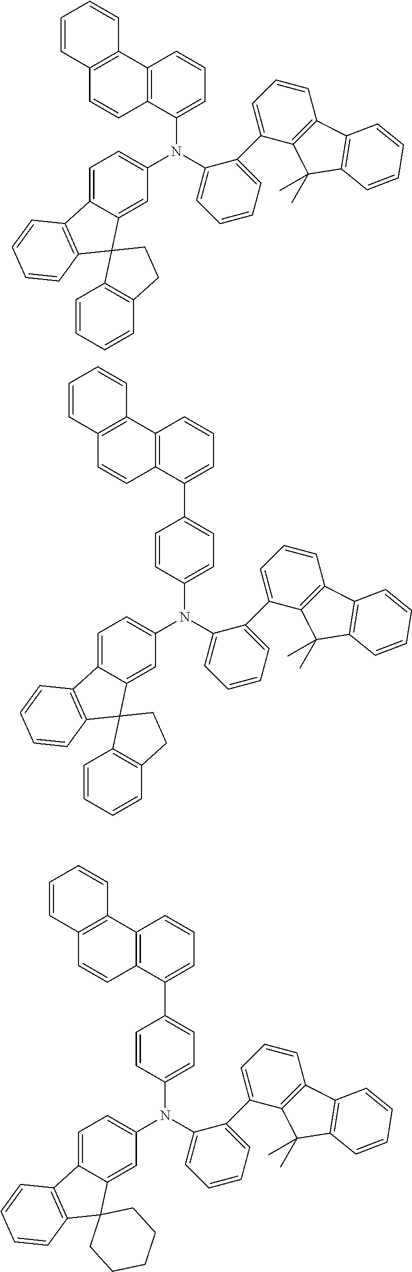

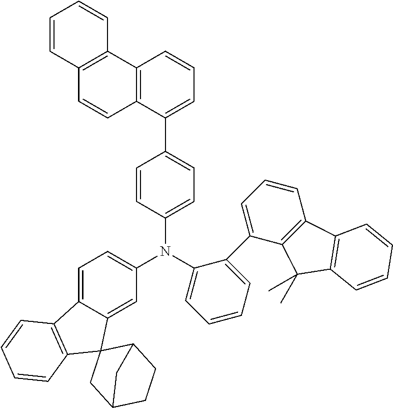 [SMILES: CC1(C)c2ccccc2-c2cccc(-c3ccccc3N(c3ccc(-c4cccc5c4ccc4ccccc45)cc3)c3ccc4c(c3)C3(CC5CCC3C5)c3ccccc3-4)c21]

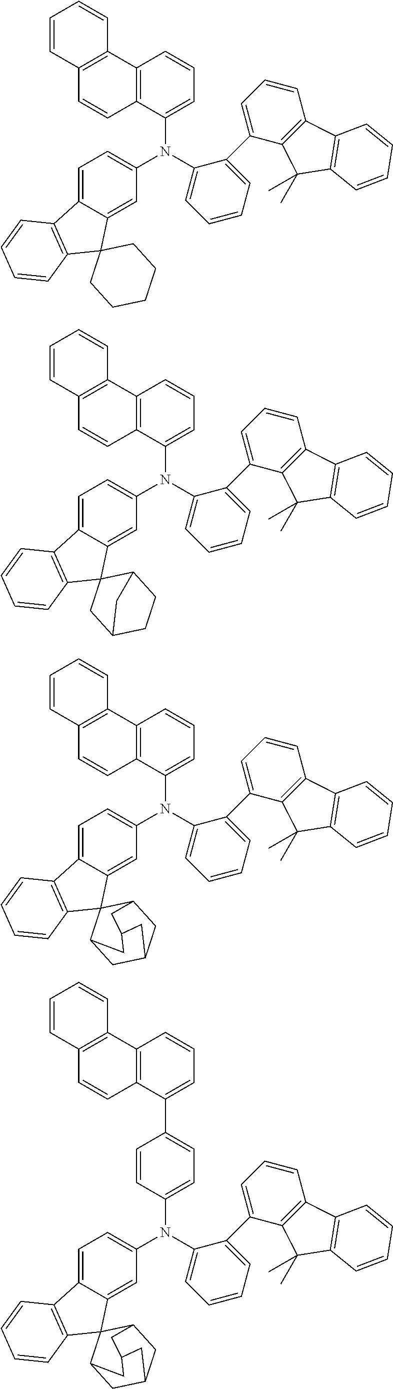

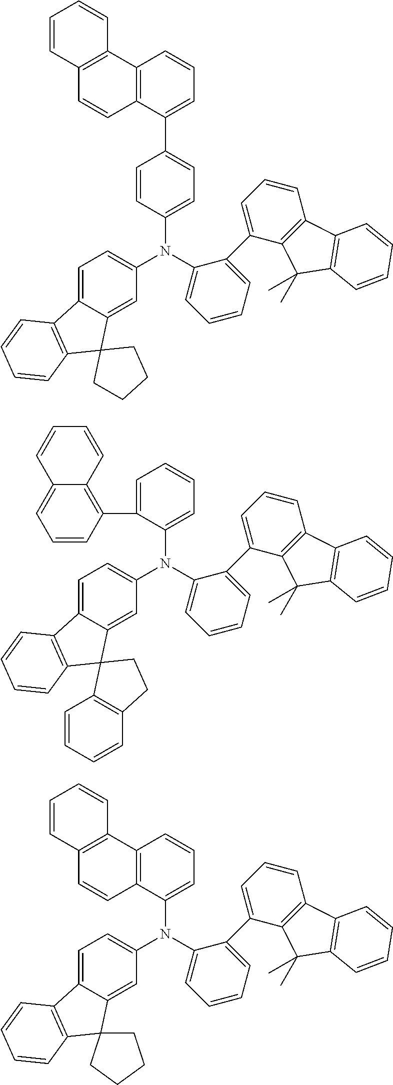

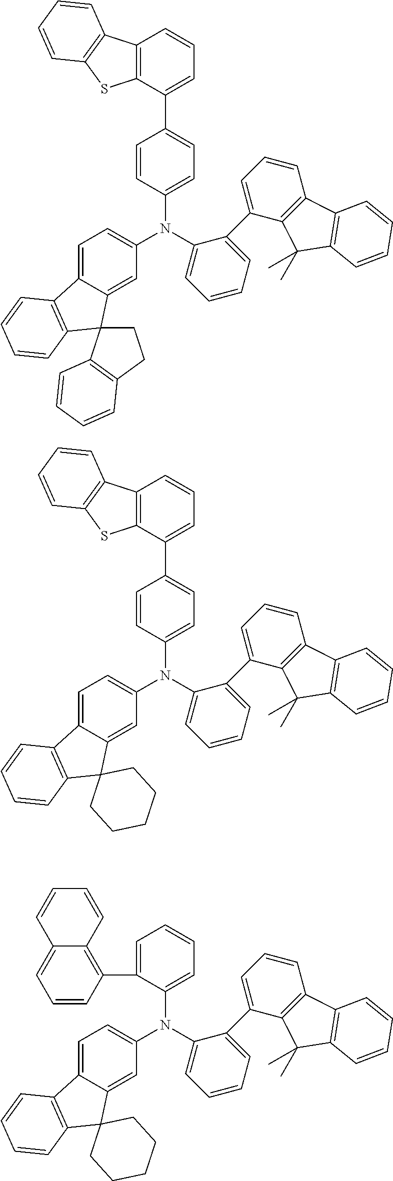

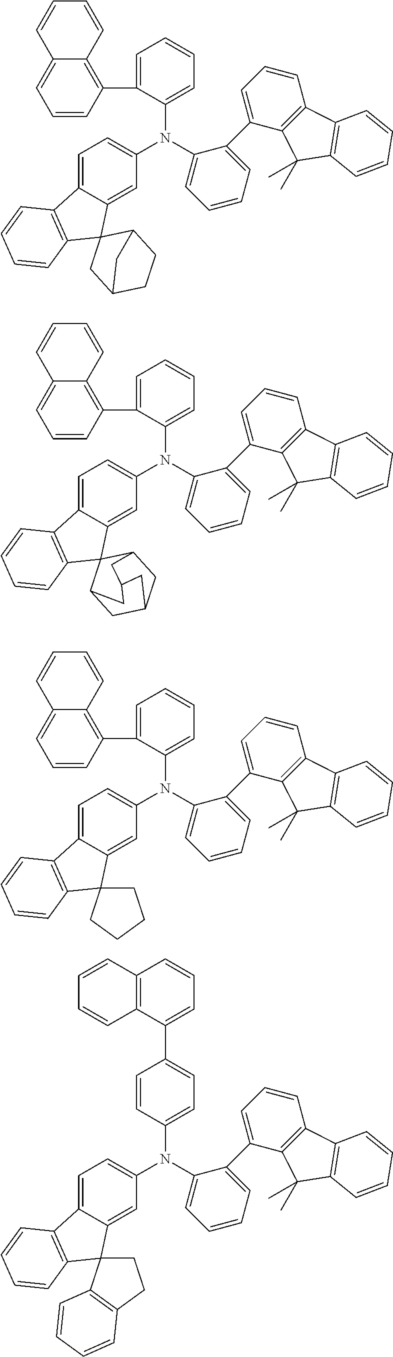

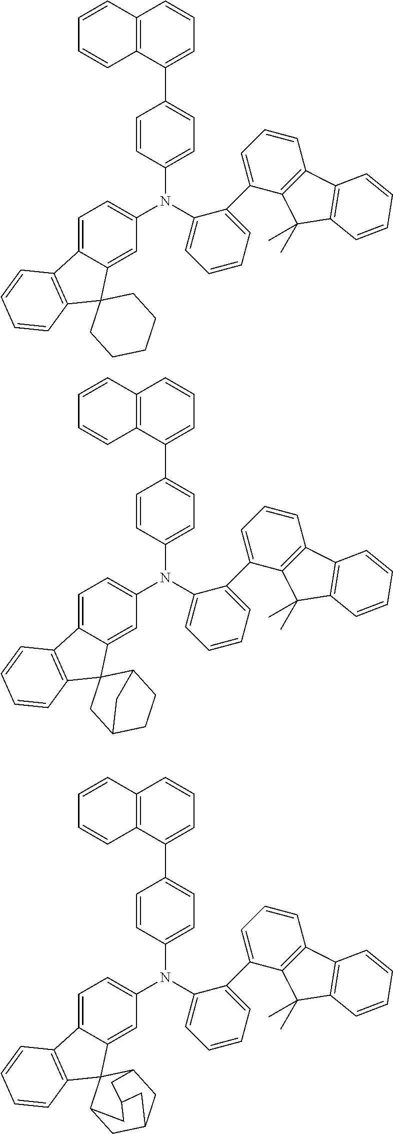

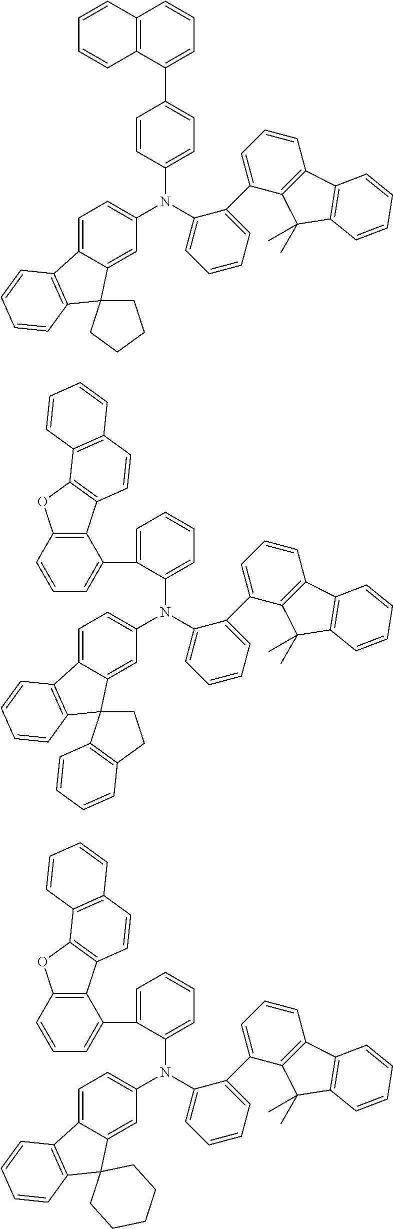

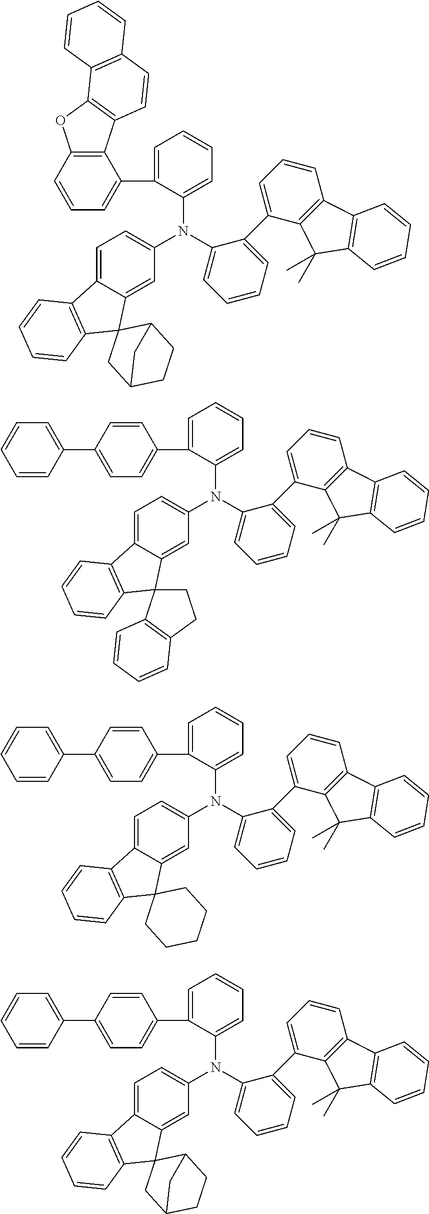

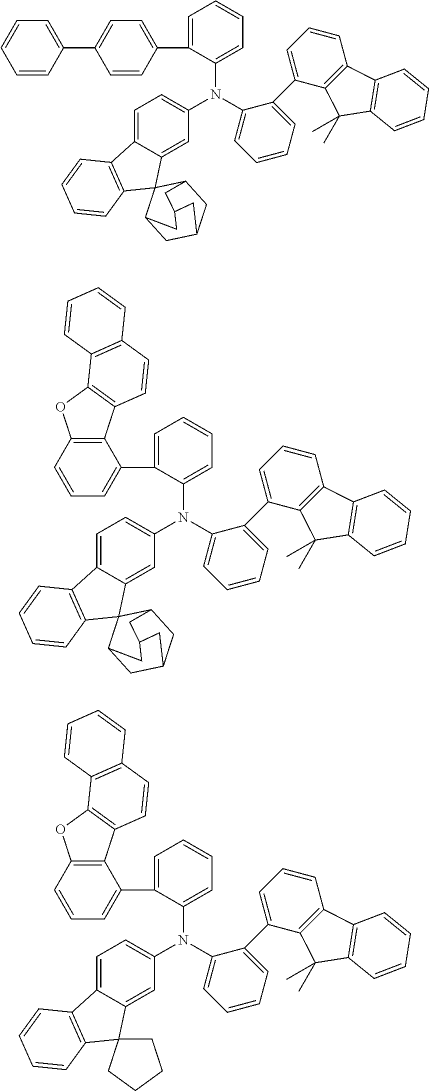 [SMILES: CC1(C)c2ccccc2-c2cccc(-c3ccccc3N(c3ccc4c(c3)C3(c5ccccc5-4)C4CC5CC(C4)CC3C5)c3ccccc3-c3ccc(-c4ccccc4)cc3)c21 ; CC1(C)c2ccccc2-c2cccc(-c3ccccc3N(c3ccc4c(c3)C3(c5ccccc5-4)C4CC5CC(C4)CC3C5)c3ccccc3-c3cccc4oc5c6ccccc6ccc5c34)c21 ; CC1(C)c2ccccc2-c2cccc(-c3ccccc3N(c3ccc4c(c3)C3(CCCC3)c3ccccc3-4)c3ccccc3-c3cccc4oc5c6ccccc6ccc5c34)c21]

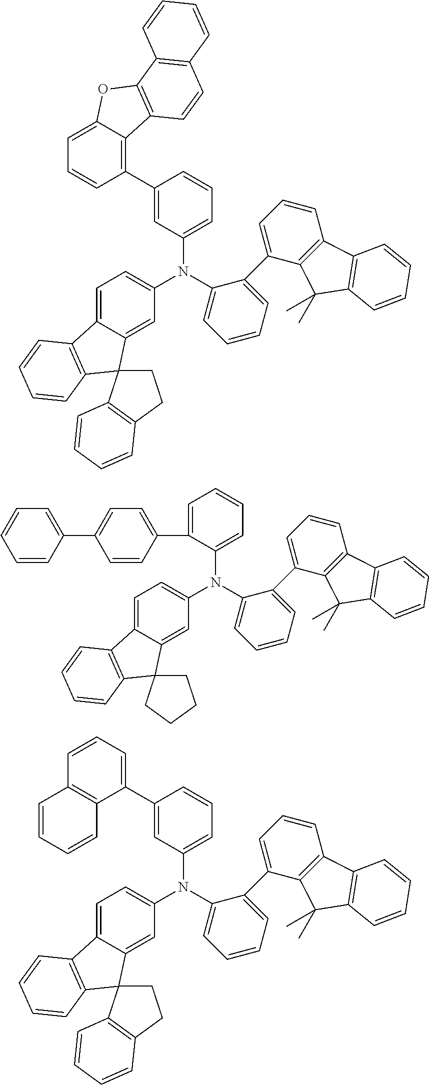

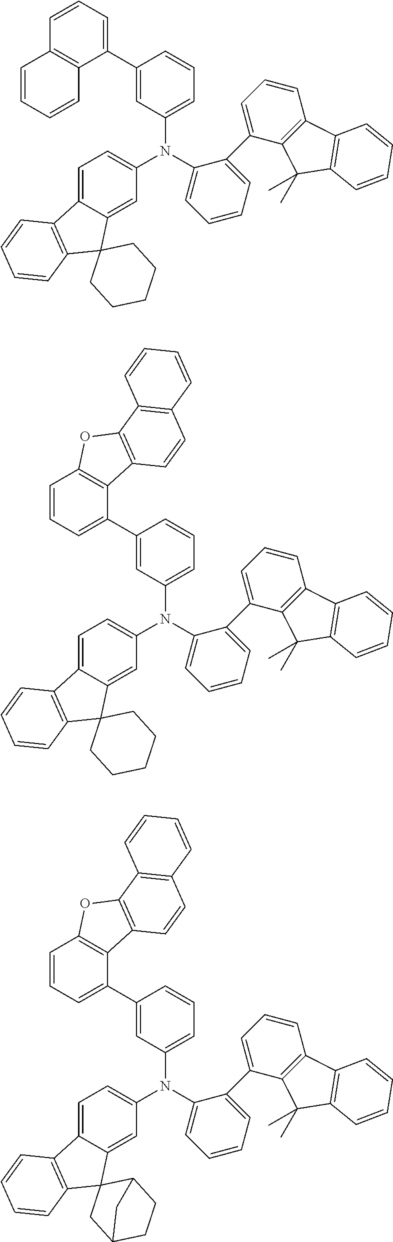 [SMILES: CC1(C)c2ccccc2-c2cccc(-c3ccccc3N(c3cccc(-c4cccc5ccccc45)c3)c3ccc4c(c3)C3(CCCCC3)c3ccccc3-4)c21 ; CC1(C)c2ccccc2-c2cccc(-c3ccccc3N(c3cccc(-c4cccc5oc6c7ccccc7ccc6c45)c3)c3ccc4c(c3)C3(CCCCC3)c3ccccc3-4)c21 ; CC1(C)c2ccccc2-c2cccc(-c3ccccc3N(c3cccc(-c4cccc5oc6c7ccccc7ccc6c45)c3)c3ccc4c(c3)C3(CC5CCC3C5)c3ccccc3-4)c21]

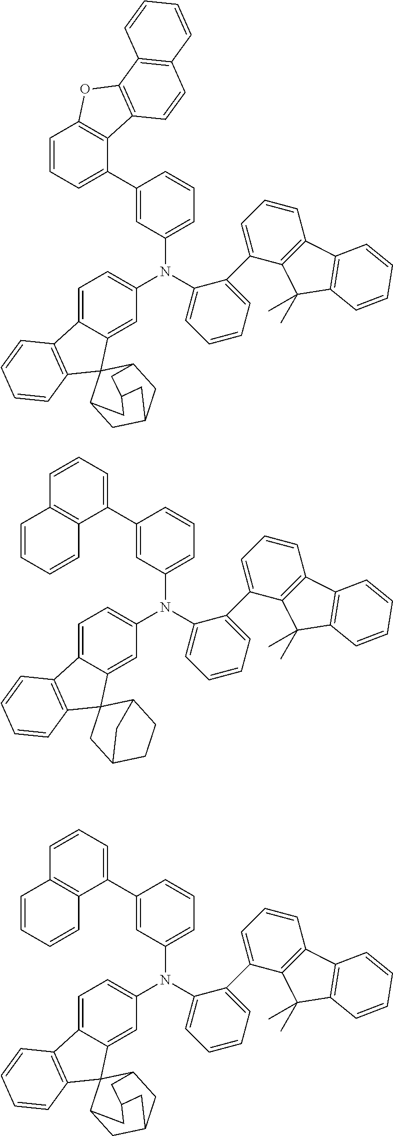

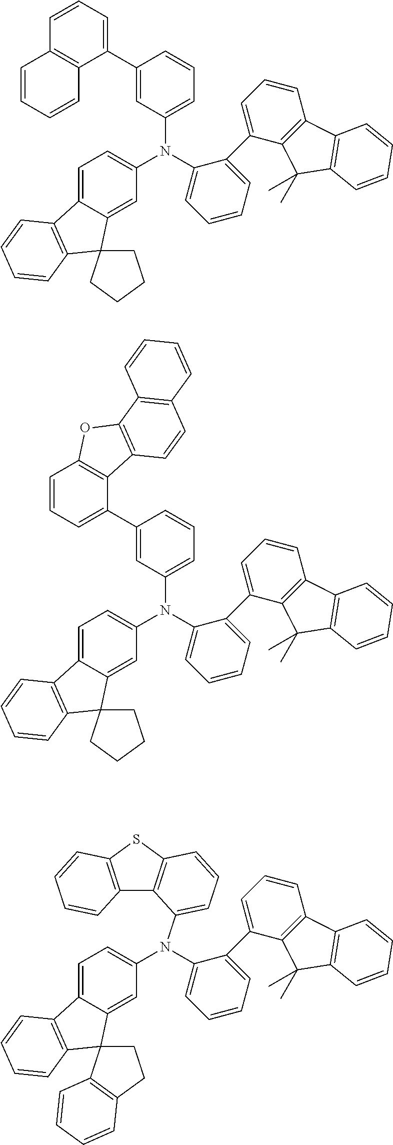 [SMILES: CC1(C)c2ccccc2-c2cccc(-c3ccccc3N(c3cccc(-c4cccc5ccccc45)c3)c3ccc4c(c3)C3(CCCC3)c3ccccc3-4)c21 ; CC1(C)c2ccccc2-c2cccc(-c3ccccc3N(c3cccc(-c4cccc5oc6c7ccccc7ccc6c45)c3)c3ccc4c(c3)C3(CCCC3)c3ccccc3-4)c21 ; CC1(C)c2ccccc2-c2cccc(-c3ccccc3N(c3ccc4c(c3)C3(CCc5ccccc53)c3ccccc3-4)c3cccc4sc5ccccc5c34)c21]

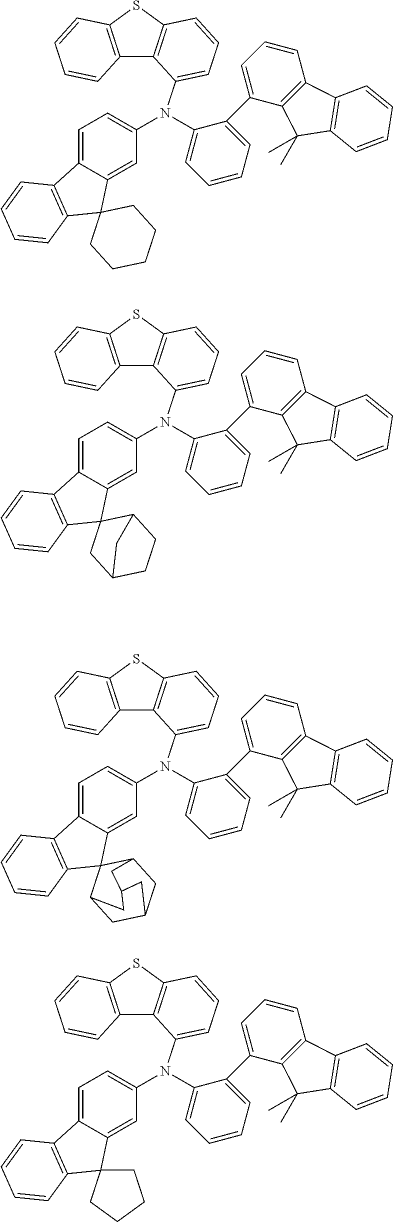 [SMILES: CC1(C)c2ccccc2-c2cccc(-c3ccccc3N(c3ccc4c(c3)C3(CCCCC3)c3ccccc3-4)c3cccc4sc5ccccc5c34)c21 ; CC1(C)c2ccccc2-c2cccc(-c3ccccc3N(c3ccc4c(c3)C3(CC5CCC3C5)c3ccccc3-4)c3cccc4sc5ccccc5c34)c21 ; CC1(C)c2ccccc2-c2cccc(-c3ccccc3N(c3ccc4c(c3)C3(c5ccccc5-4)C4CC5CC(C4)CC3C5)c3cccc4sc5ccccc5c34)c21 ; CC1(C)c2ccccc2-c2cccc(-c3ccccc3N(c3ccc4c(c3)C3(CCCC3)c3ccccc3-4)c3cccc4sc5ccccc5c34)c21]

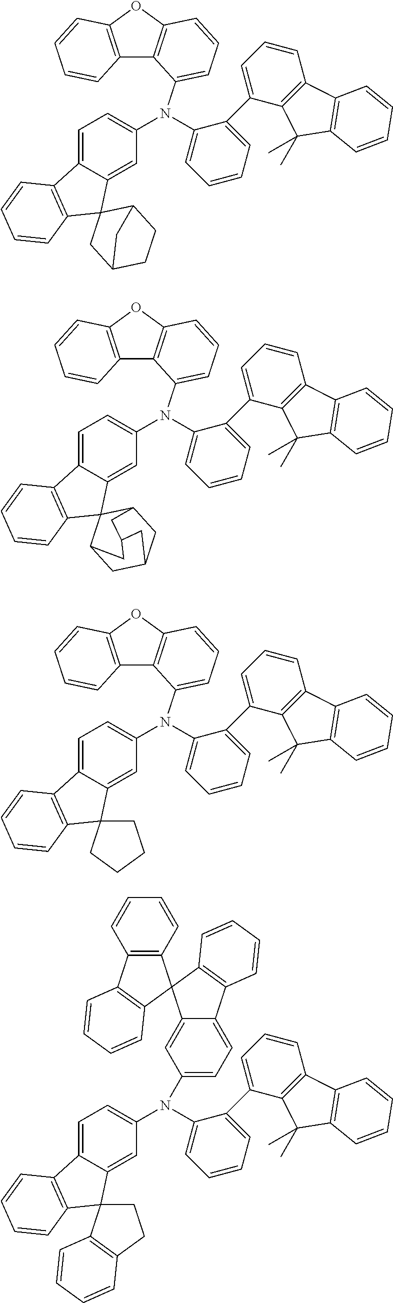

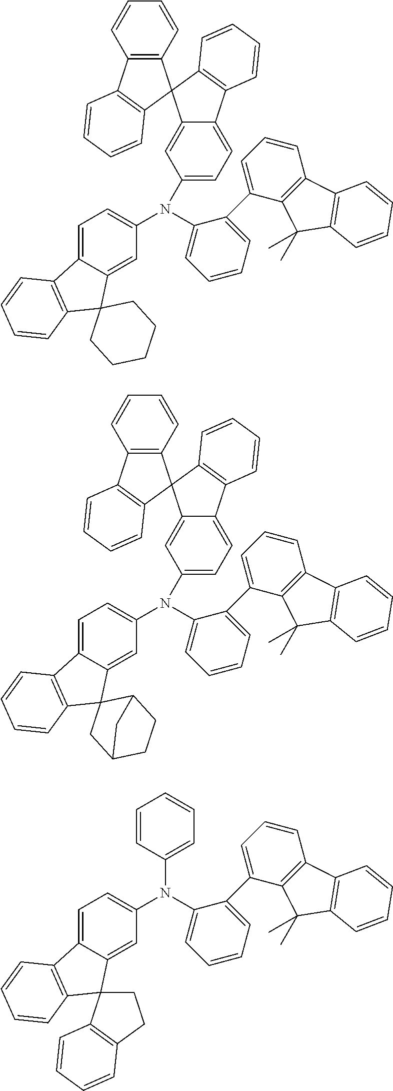

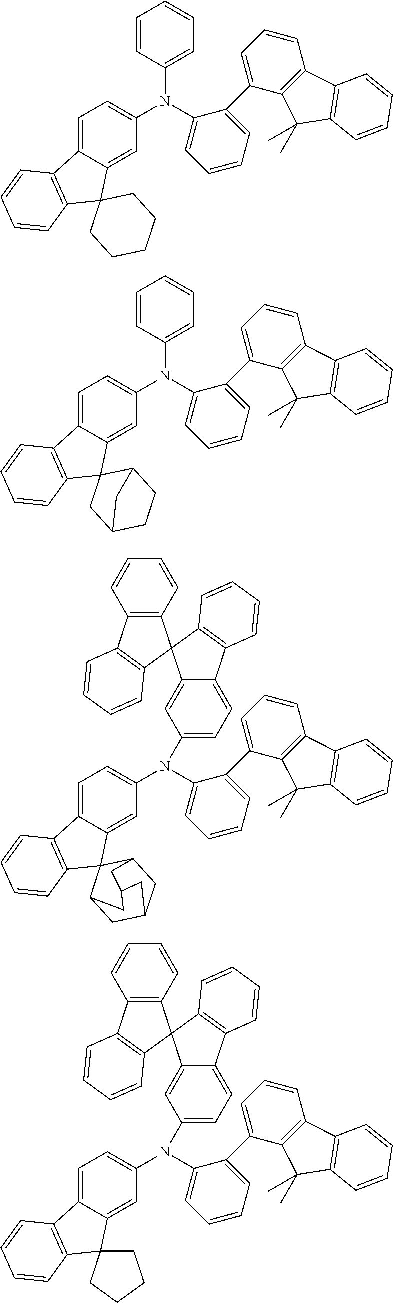

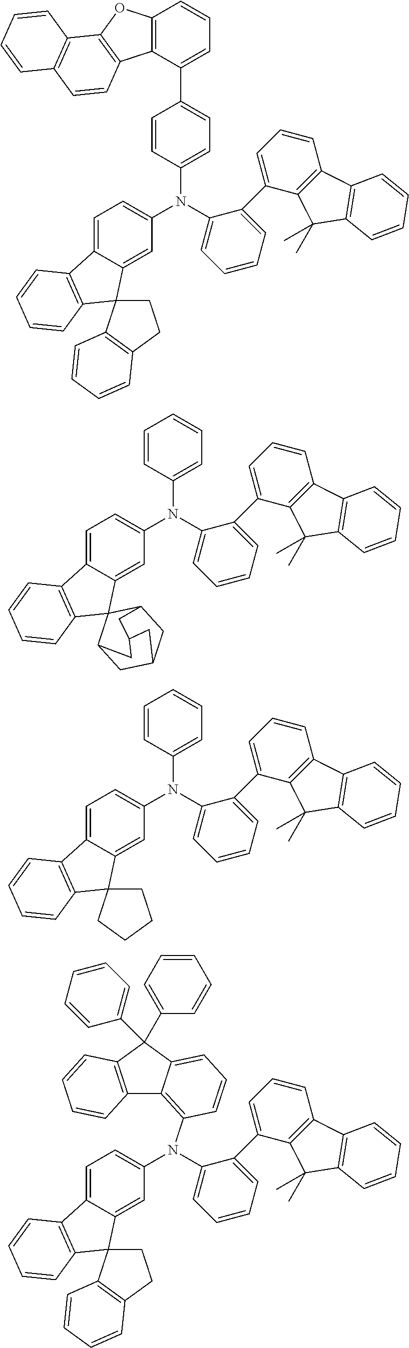

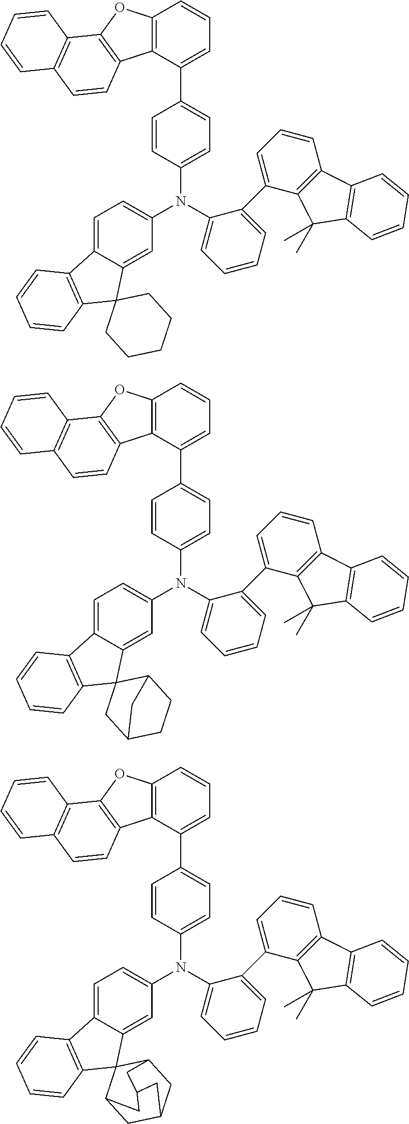

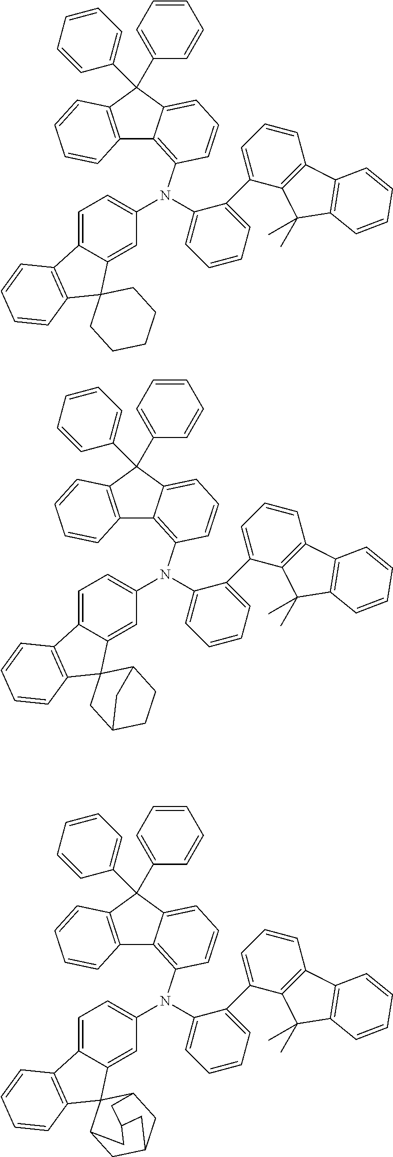

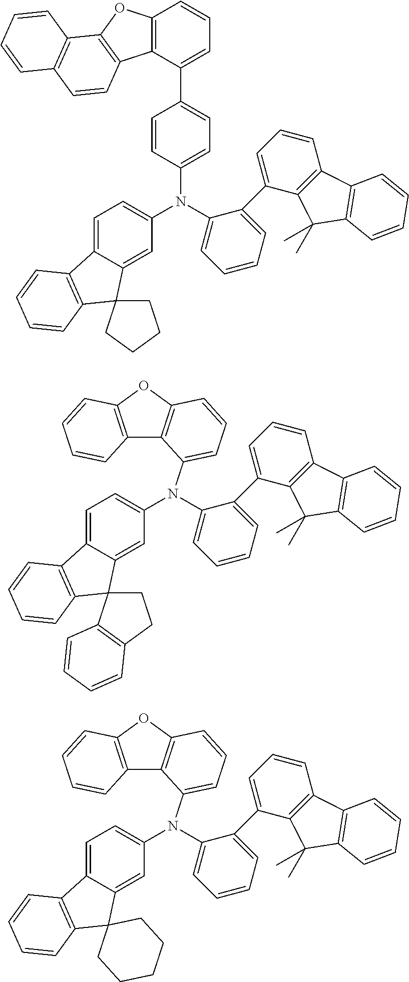

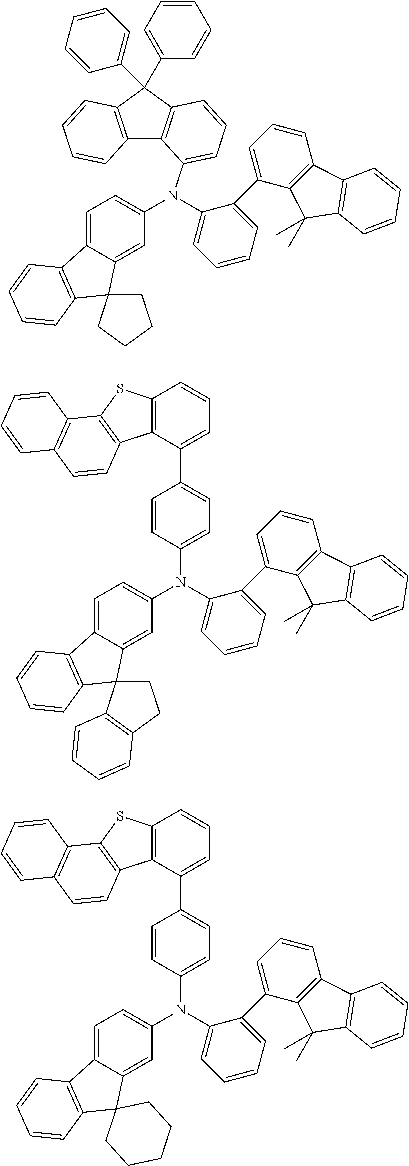

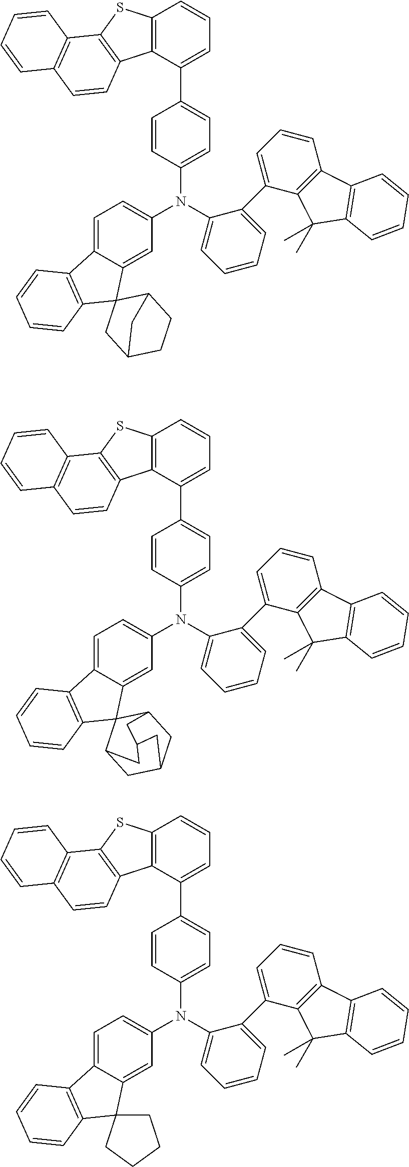 [SMILES: CC1(C)c2ccccc2-c2cccc(-c3ccccc3N(c3ccc(-c4cccc5sc6c7ccccc7ccc6c45)cc3)c3ccc4c(c3)C3(CC5CCC3C5)c3ccccc3-4)c21 ; CC1(C)c2ccccc2-c2cccc(-c3ccccc3N(c3ccc(-c4cccc5sc6c7ccccc7ccc6c45)cc3)c3ccc4c(c3)C3(c5ccccc5-4)C4CC5CC(C4)CC3C5)c21 ; CC1(C)c2ccccc2-c2cccc(-c3ccccc3N(c3ccc(-c4cccc5sc6c7ccccc7ccc6c45)cc3)c3ccc4c(c3)C3(CCCC3)c3ccccc3-4)c21]

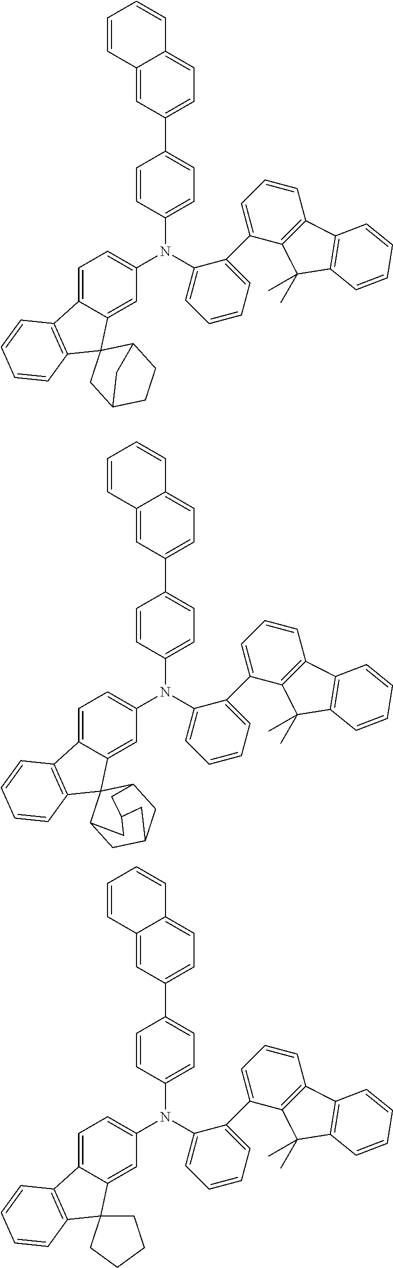

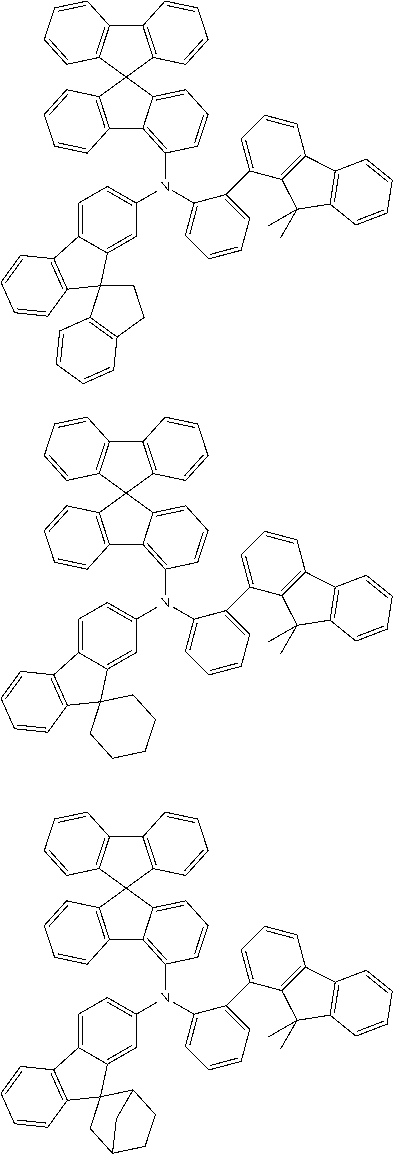

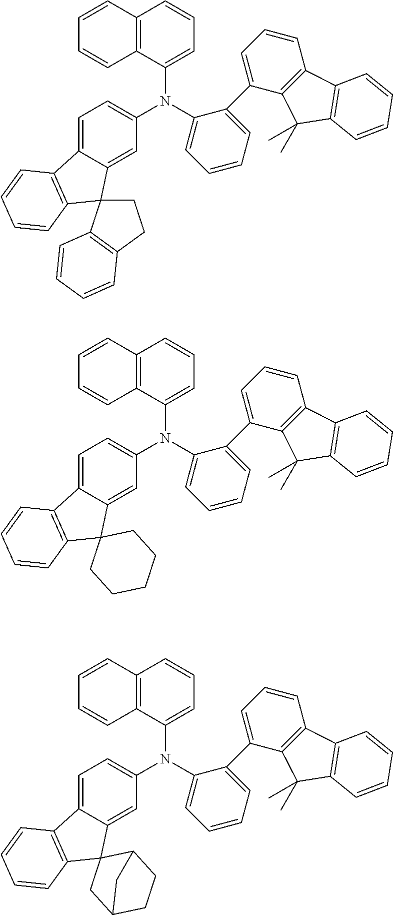

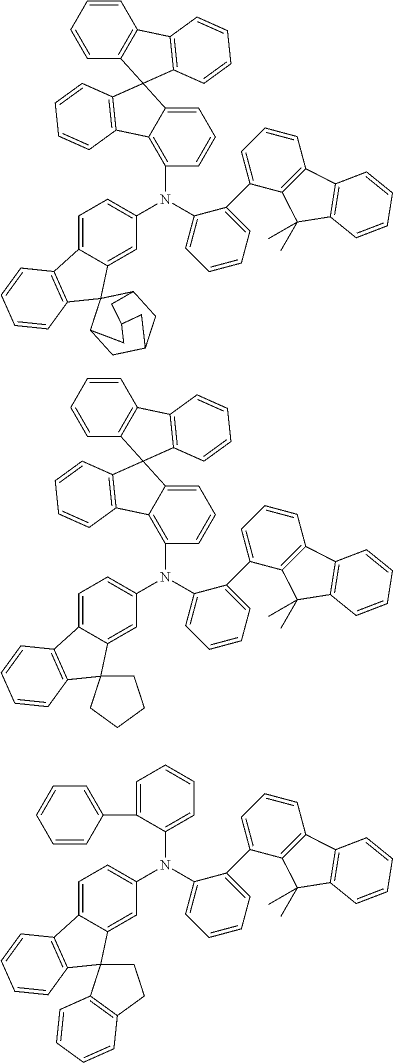

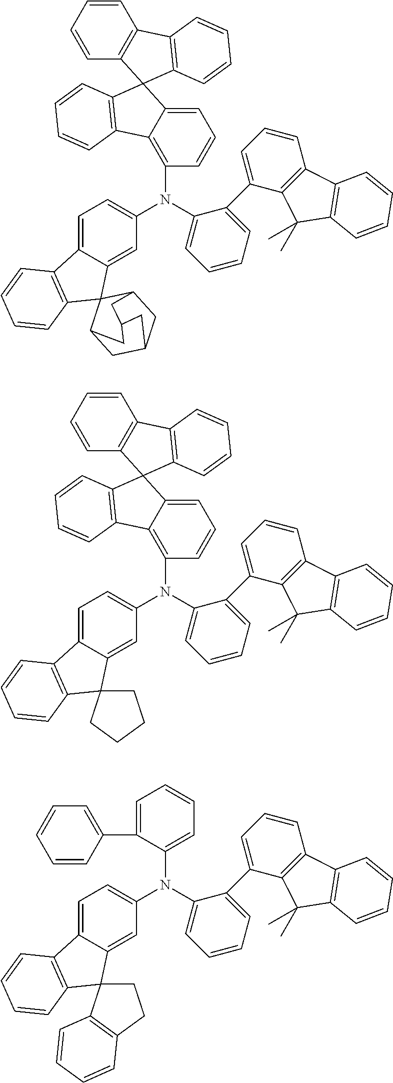

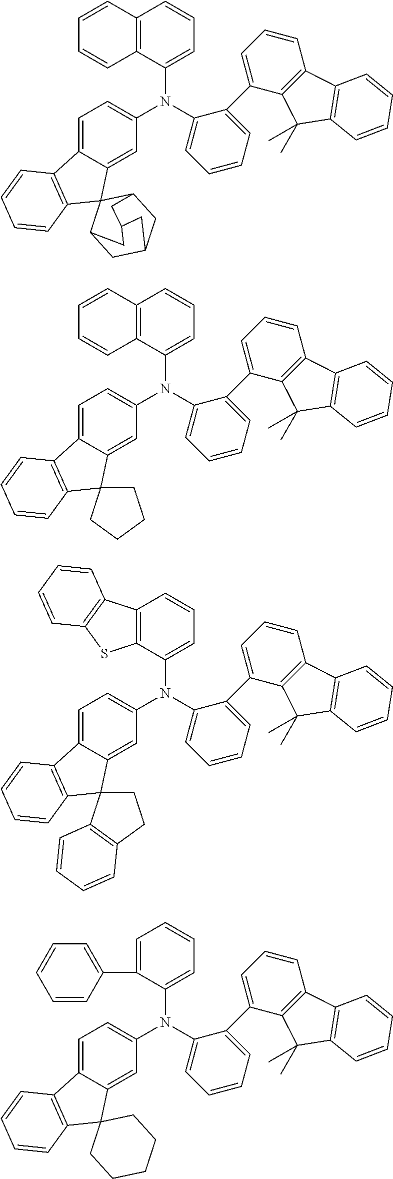

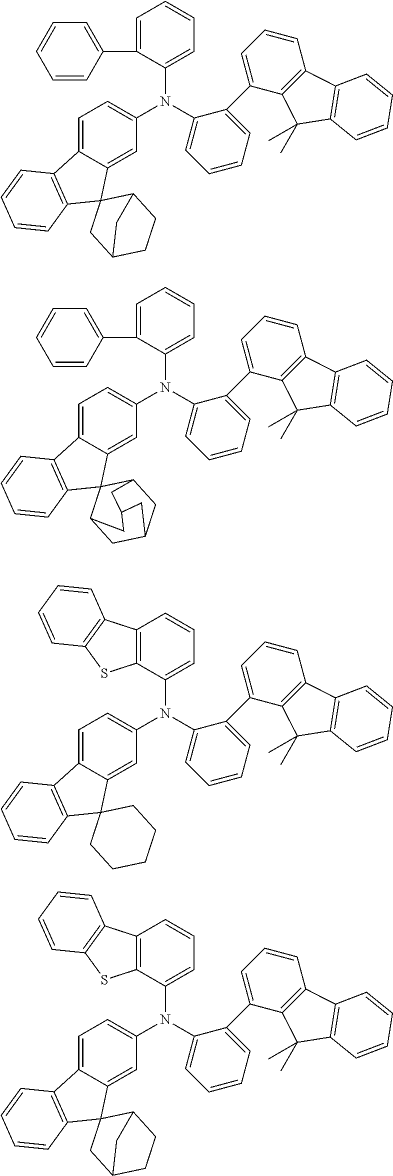

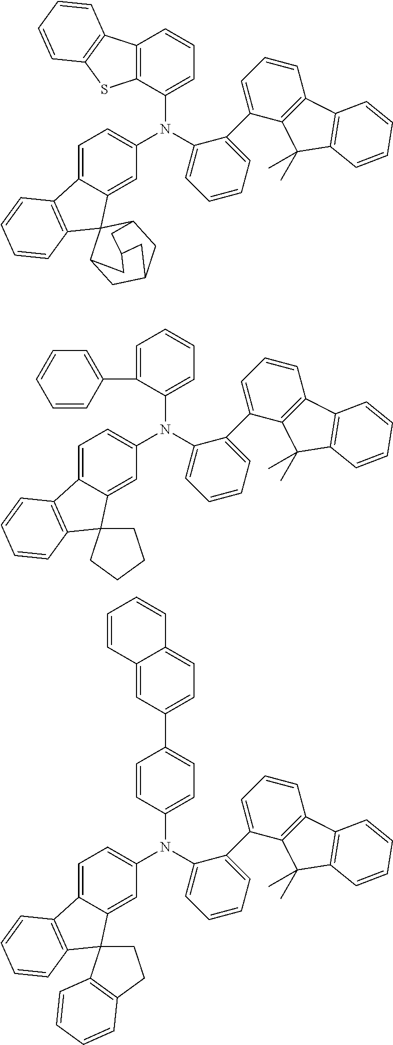

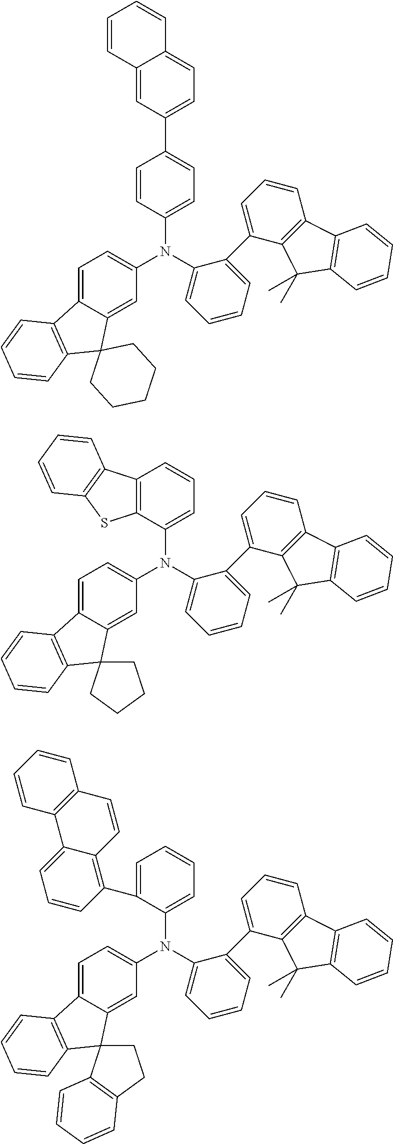

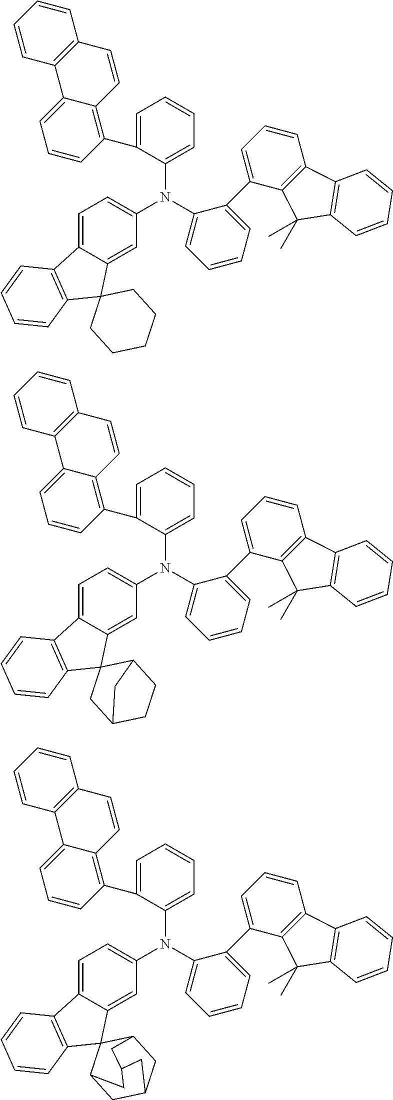

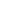

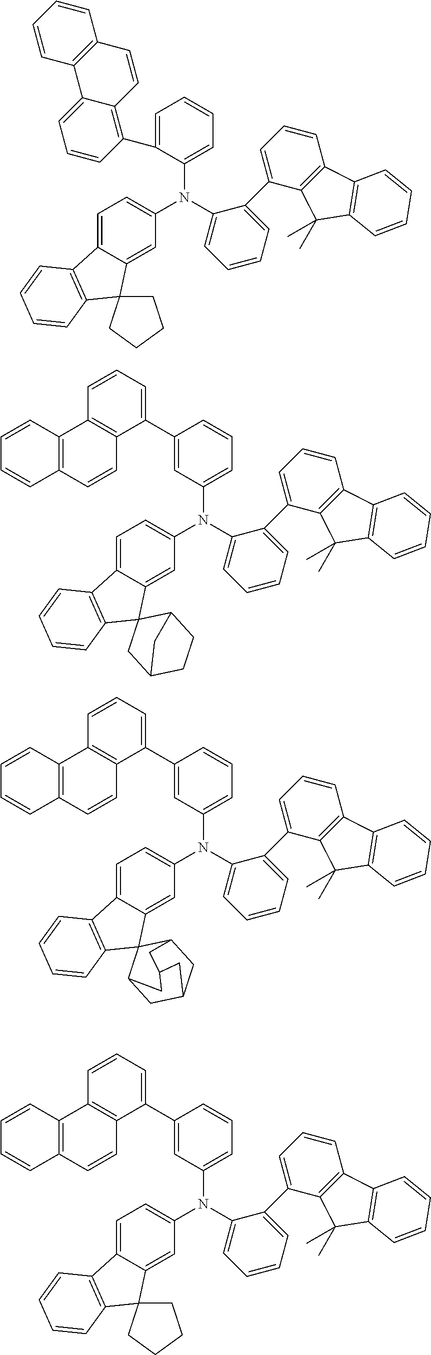

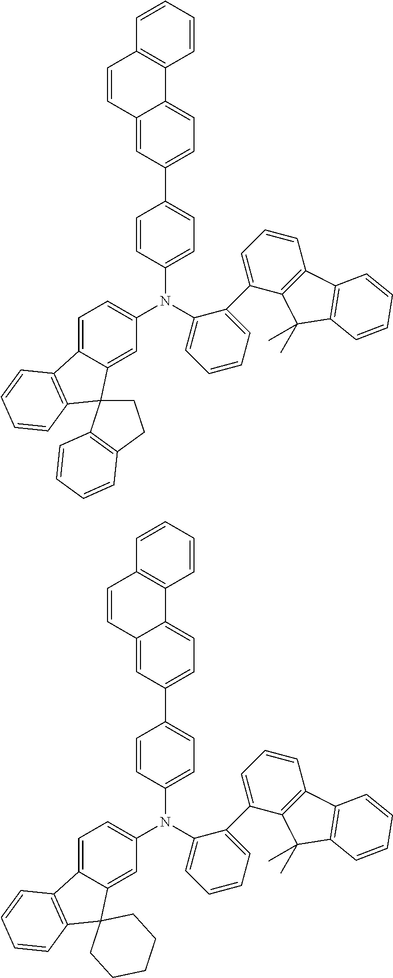 [SMILES: CC1(C)c2ccccc2-c2cccc(-c3ccccc3N(c3ccc(-c4ccc5c(ccc6ccccc65)c4)cc3)c3ccc4c(c3)C3(CCc5ccccc53)c3ccccc3-4)c21 ; CC1(C)c2ccccc2-c2cccc(-c3ccccc3N(c3ccc(-c4ccc5c(ccc6ccccc65)c4)cc3)c3ccc4c(c3)C3(CCCCC3)c3ccccc3-4)c21]

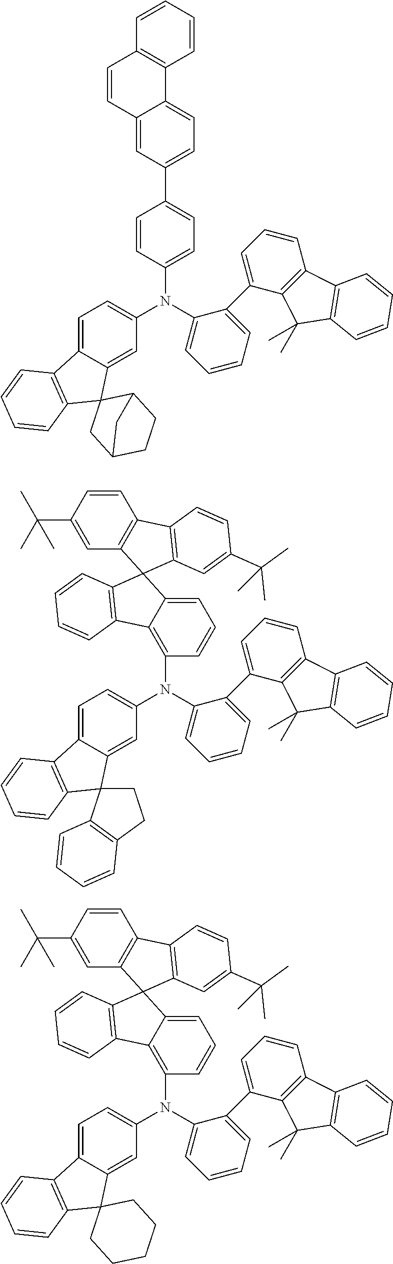

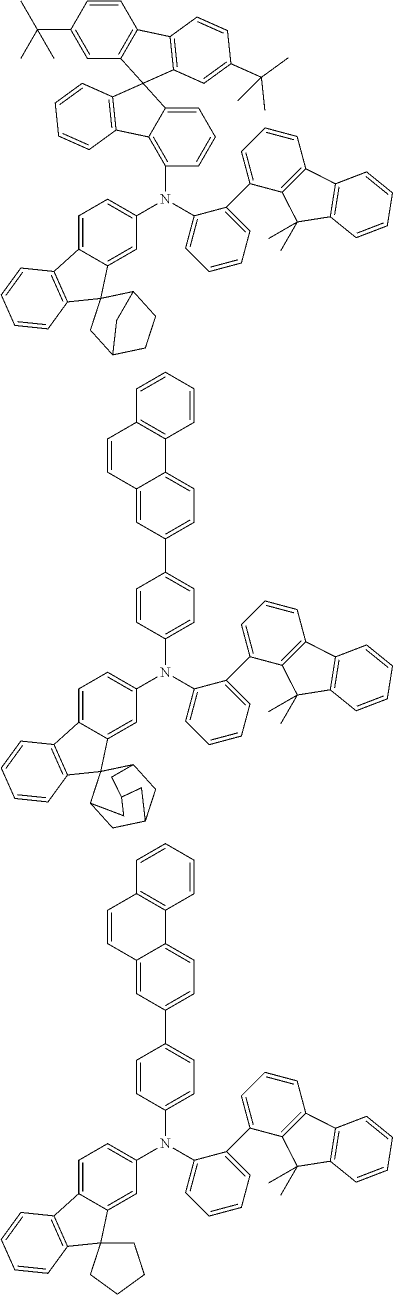

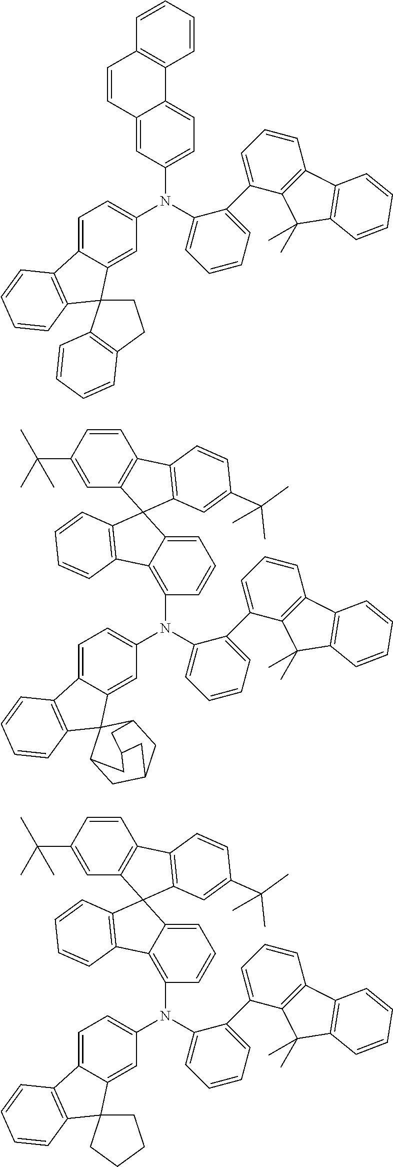

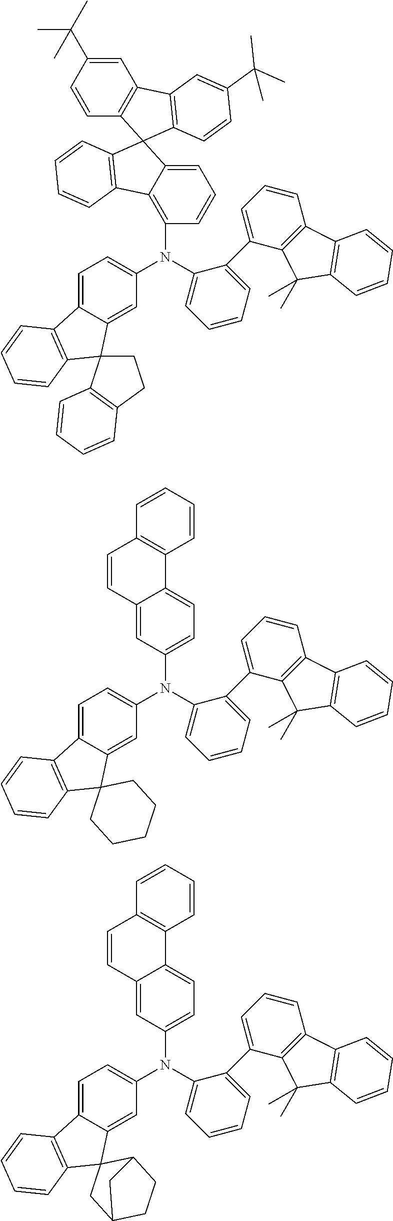

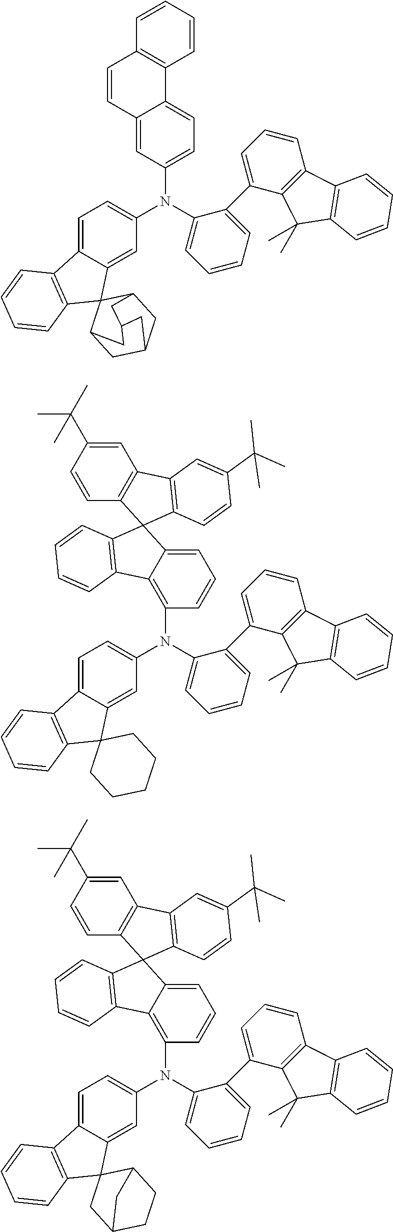

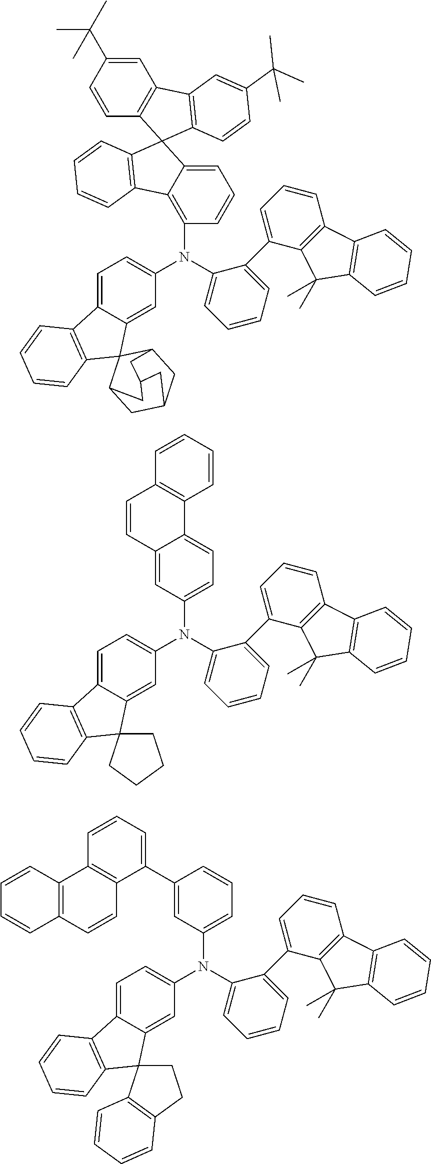

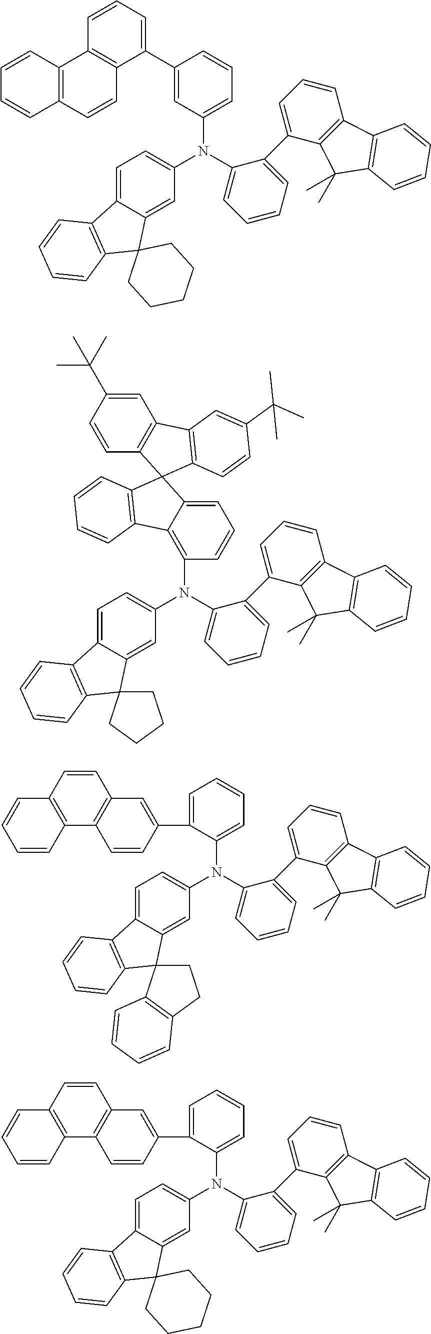

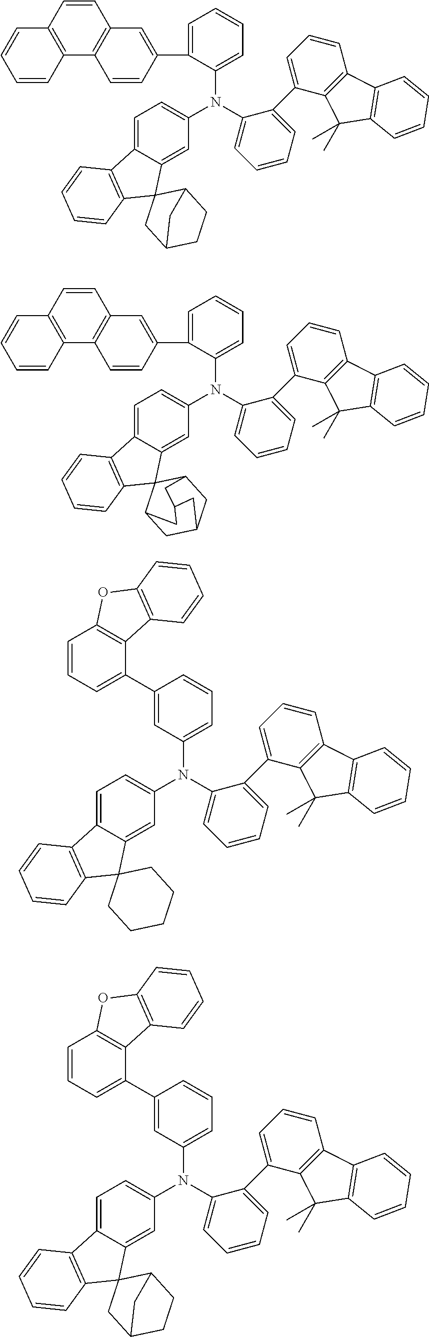

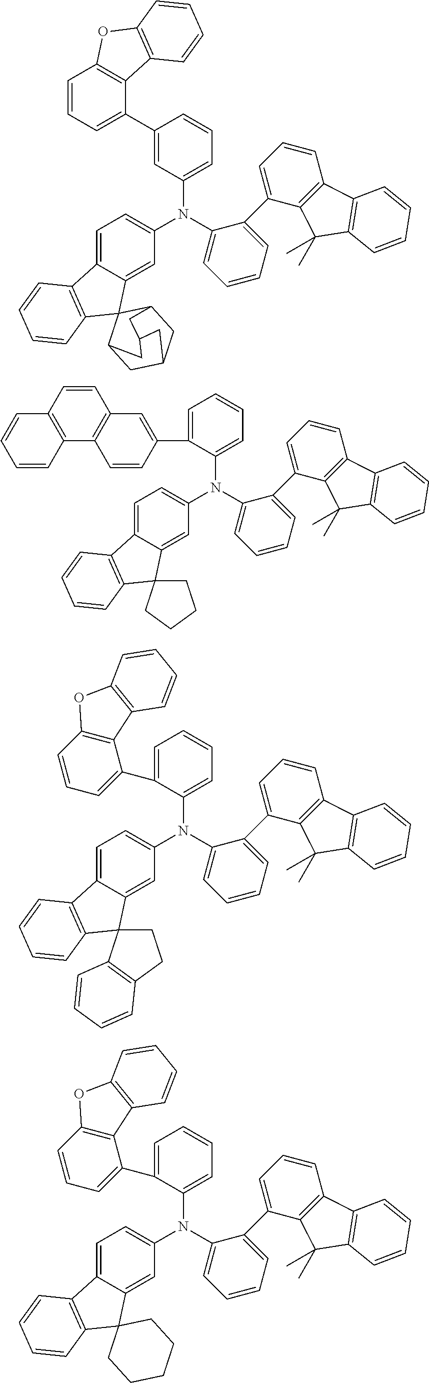

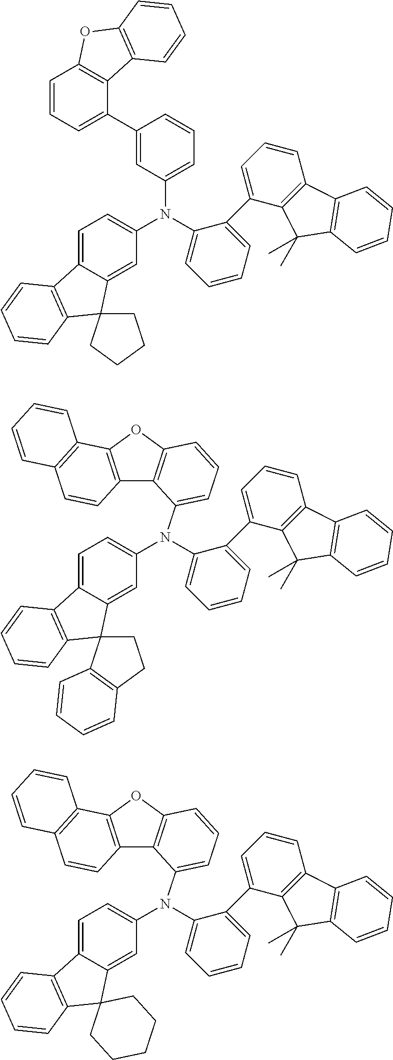

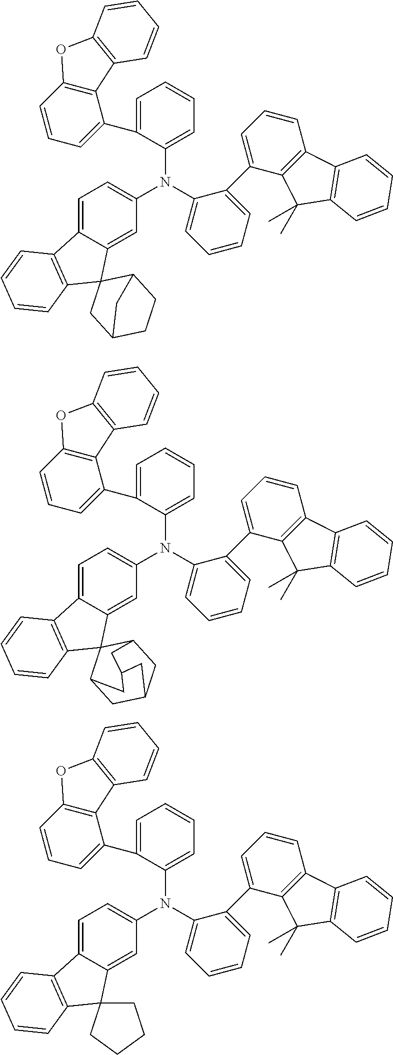

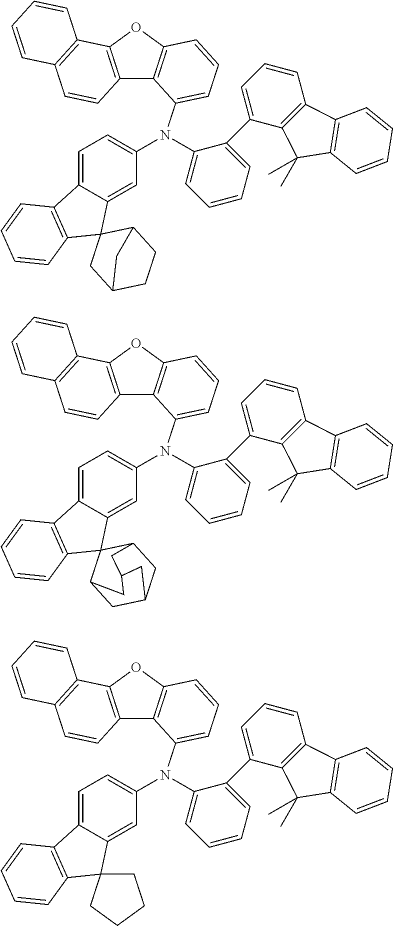 [SMILES: CC1(C)c2ccccc2-c2cccc(-c3ccccc3N(c3ccc4c(c3)C3(CC5CCC3C5)c3ccccc3-4)c3cccc4oc5c6ccccc6ccc5c34)c21 ; CC1(C)c2ccccc2-c2cccc(-c3ccccc3N(c3ccc4c(c3)C3(c5ccccc5-4)C4CC5CC(C4)CC3C5)c3cccc4oc5c6ccccc6ccc5c34)c21 ; CC1(C)c2ccccc2-c2cccc(-c3ccccc3N(c3ccc4c(c3)C3(CCCC3)c3ccccc3-4)c3cccc4oc5c6ccccc6ccc5c34)c21]

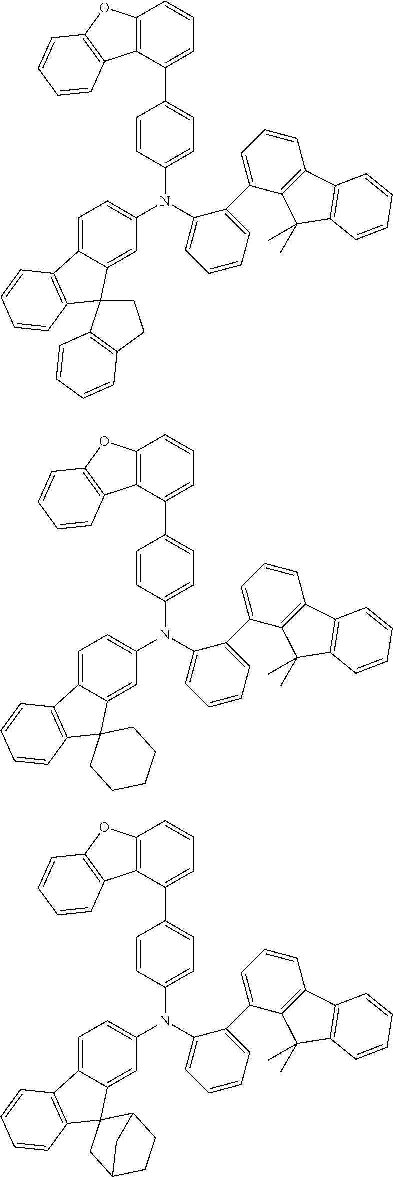

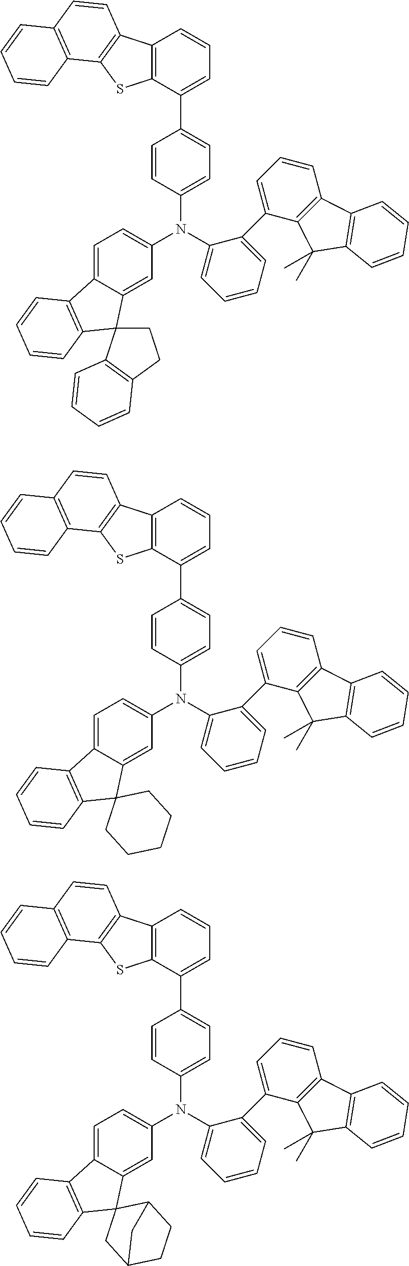

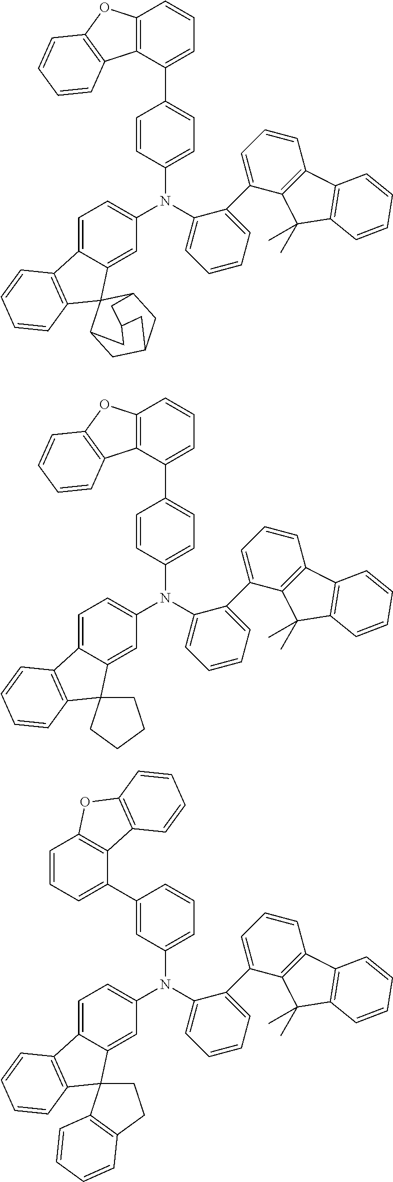

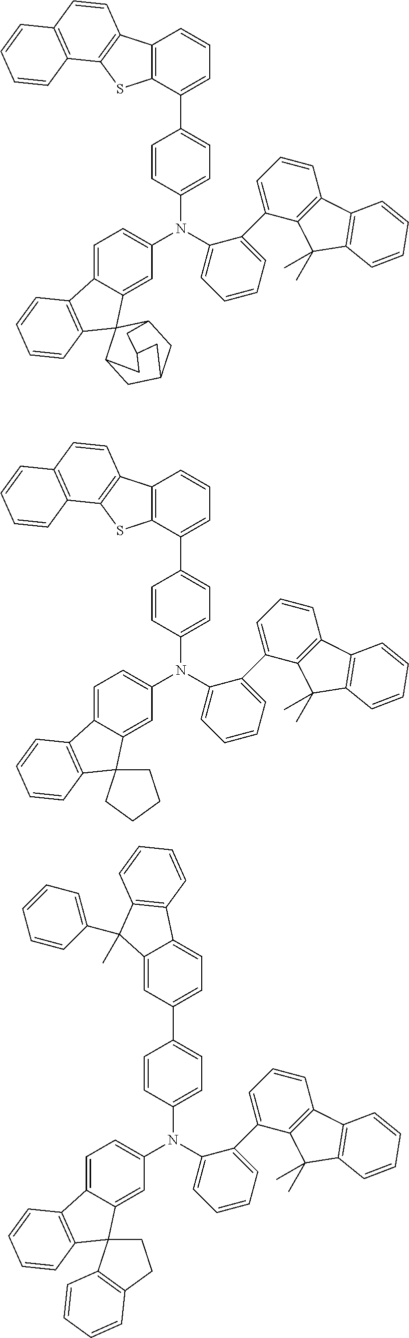

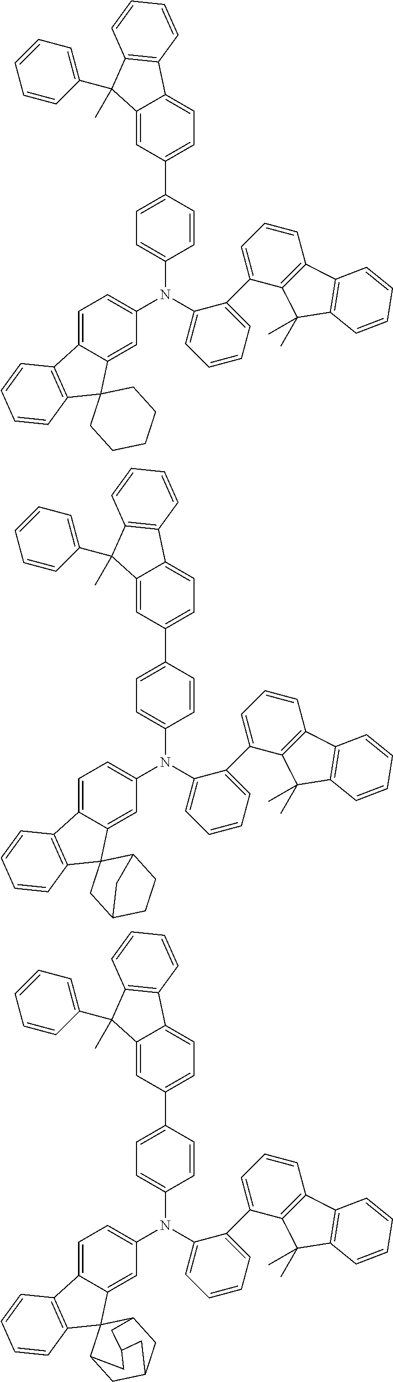

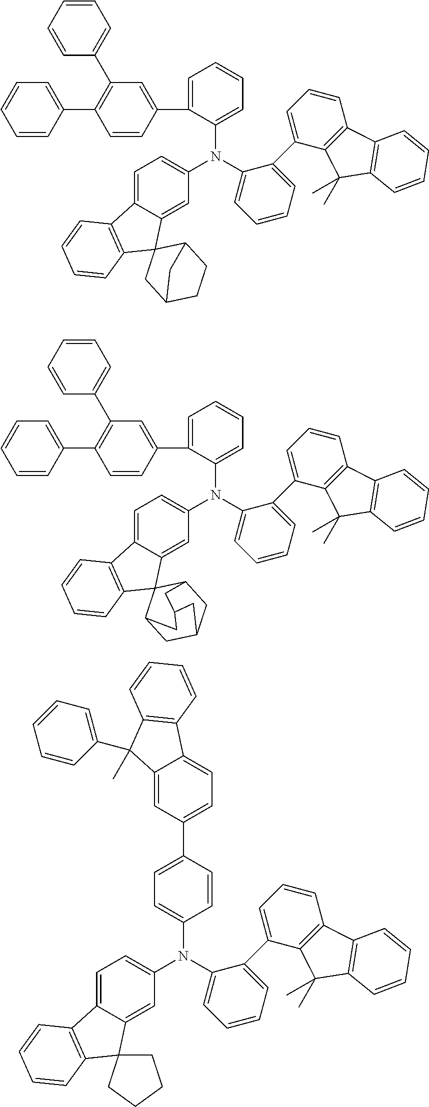

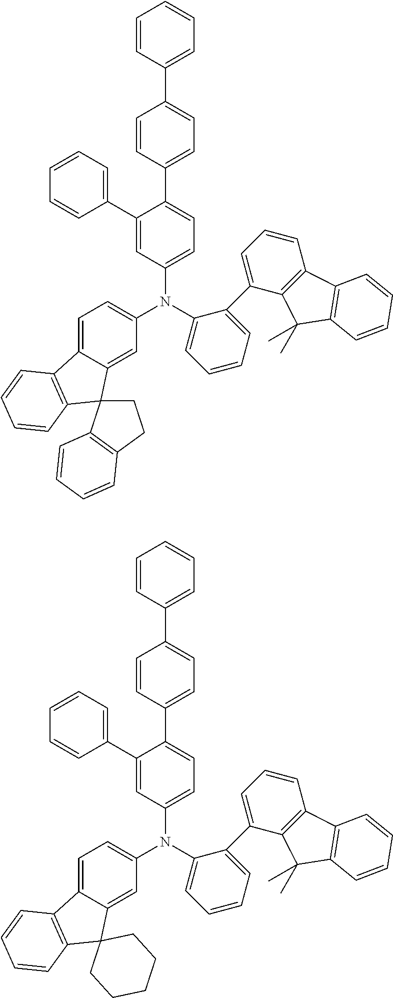 [SMILES: CC1(C)c2ccccc2-c2cccc(-c3ccccc3N(c3ccc(-c4ccc(-c5ccccc5)cc4)c(-c4ccccc4)c3)c3ccc4c(c3)C3(CCc5ccccc53)c3ccccc3-4)c21 ; CC1(C)c2ccccc2-c2cccc(-c3ccccc3N(c3ccc(-c4ccc(-c5ccccc5)cc4)c(-c4ccccc4)c3)c3ccc4c(c3)C3(CCCCC3)c3ccccc3-4)c21]

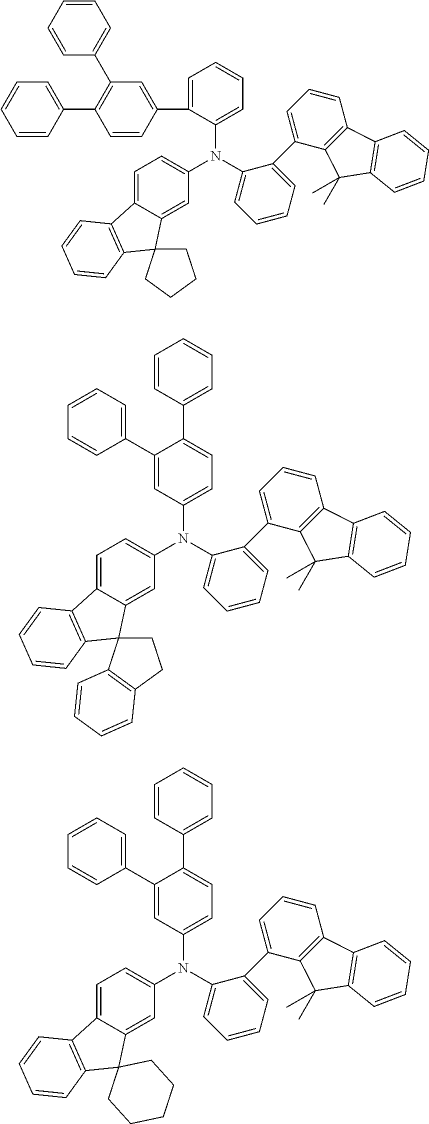

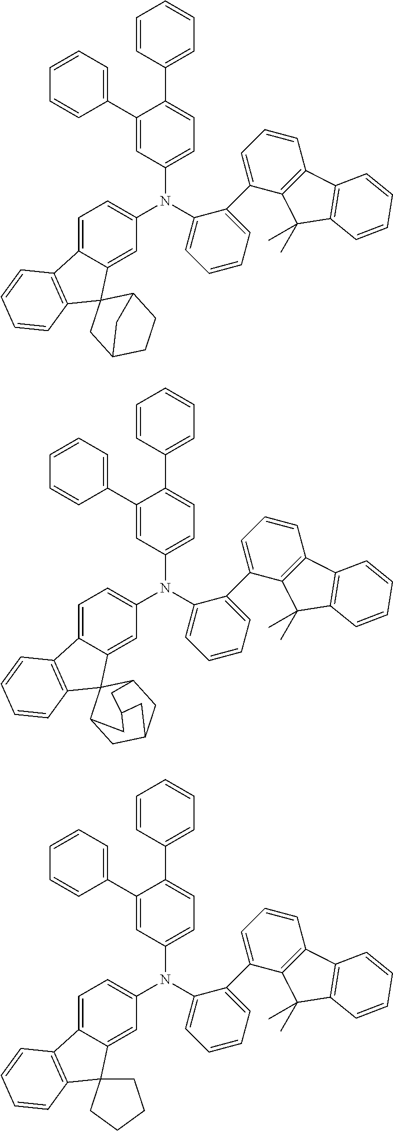

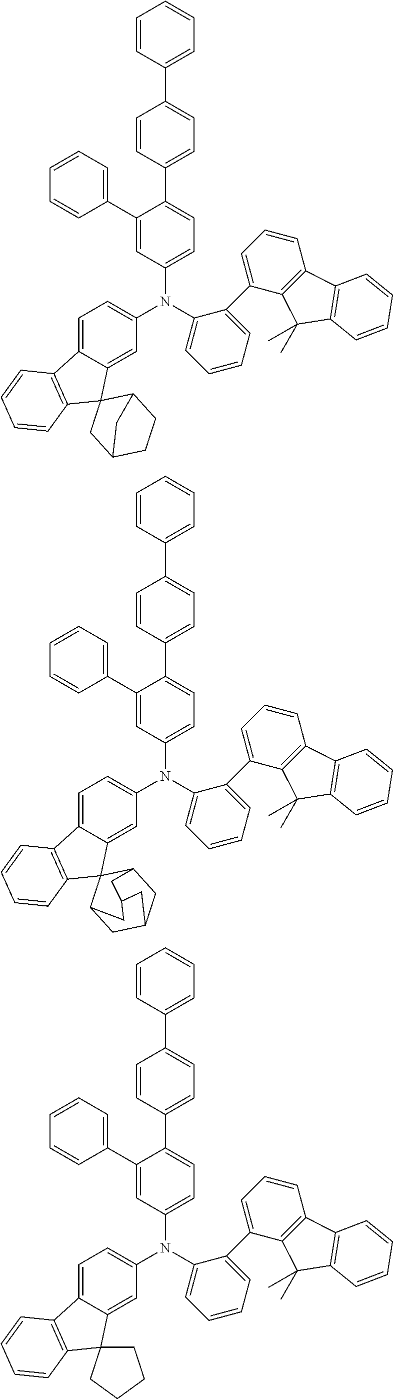

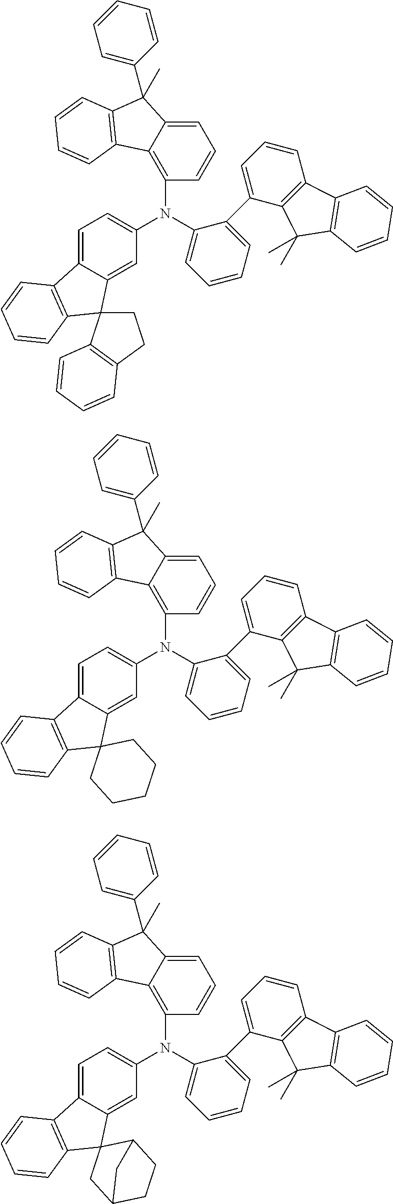

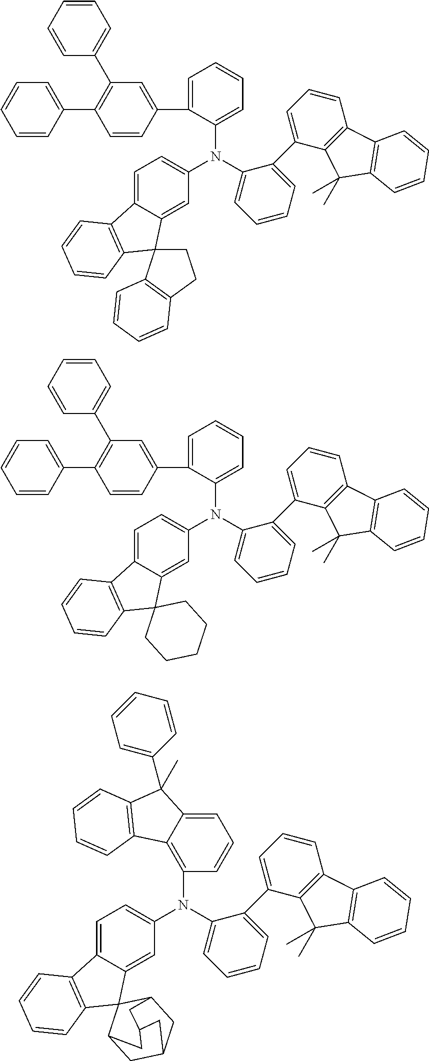

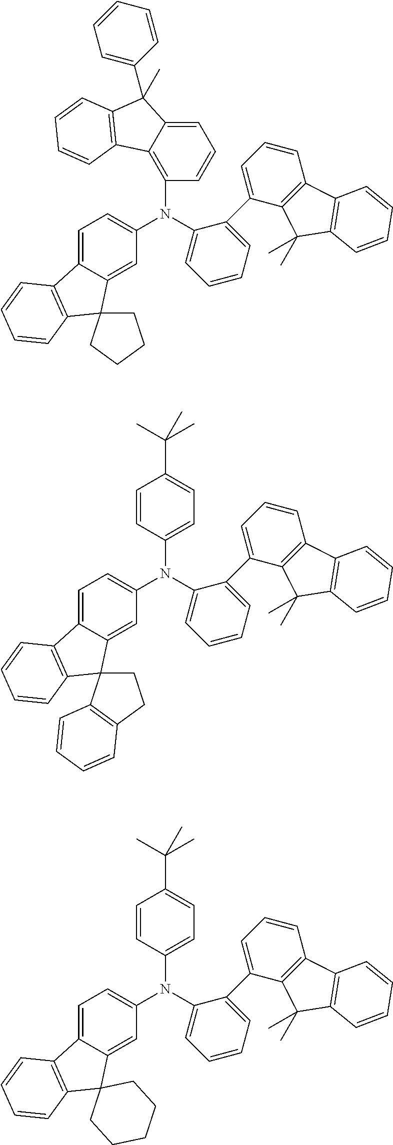 [SMILES: CC1(C)c2ccccc2-c2cccc(-c3ccccc3N(c3ccc4c(c3)C3(CCCC3)c3ccccc3-4)c3cccc4c3-c3ccccc3C4(C)c3ccccc3)c21 ; CC(C)(C)c1ccc(N(c2ccc3c(c2)C2(CCc4ccccc42)c2ccccc2-3)c2ccccc2-c2cccc3c2C(C)(C)c2ccccc2-3)cc1 ; CC(C)(C)c1ccc(N(c2ccc3c(c2)C2(CCCCC2)c2ccccc2-3)c2ccccc2-c2cccc3c2C(C)(C)c2ccccc2-3)cc1]

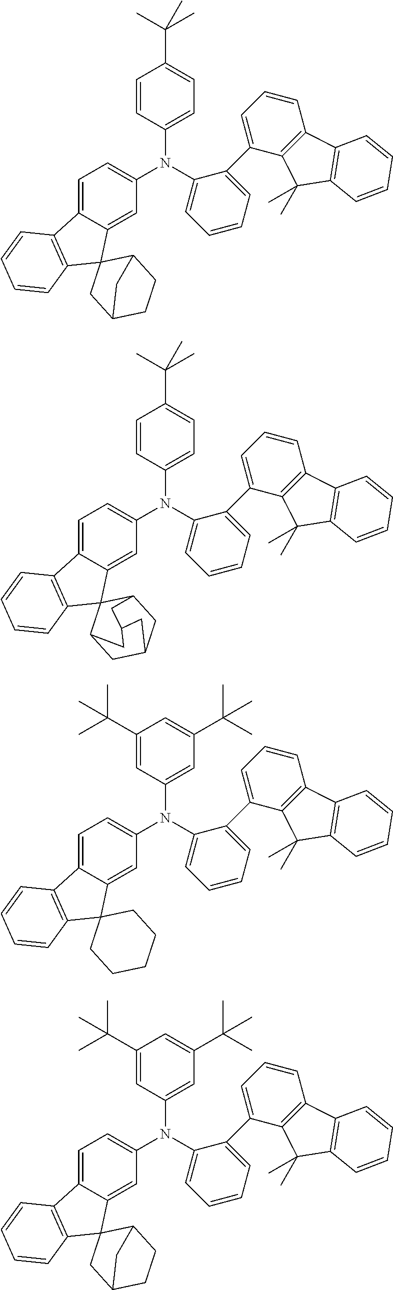

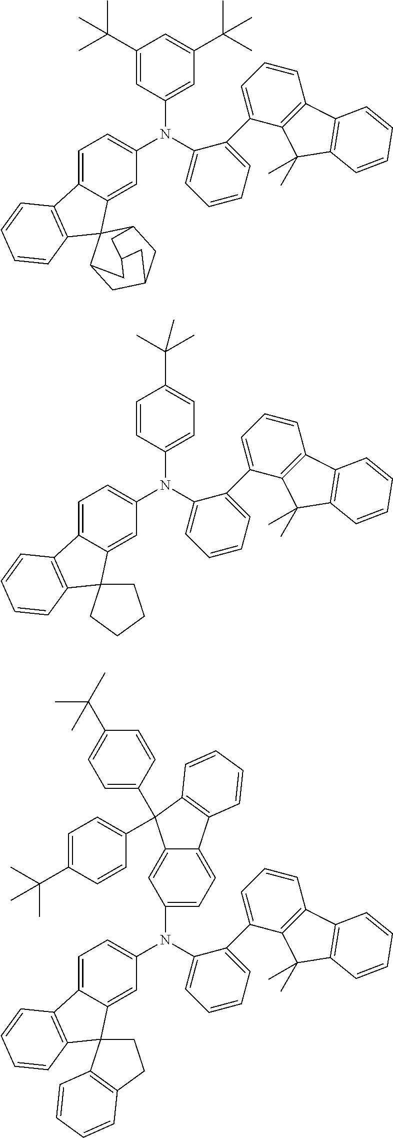

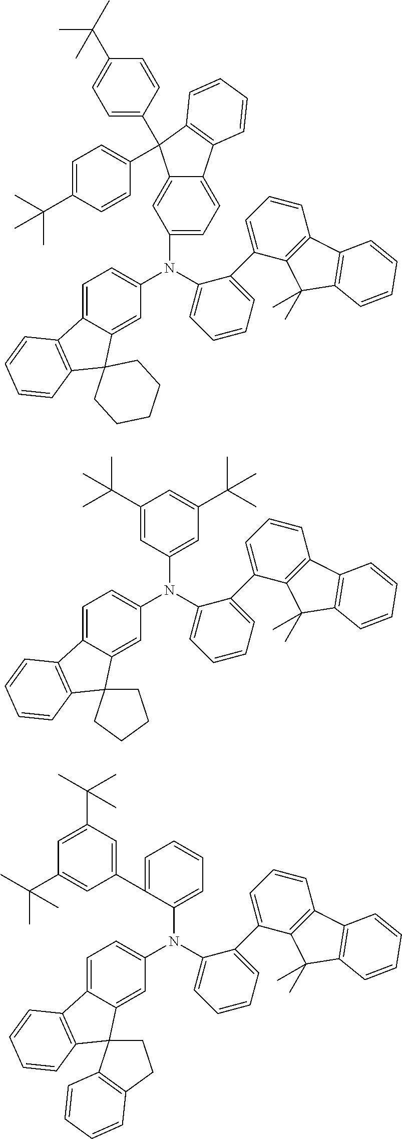

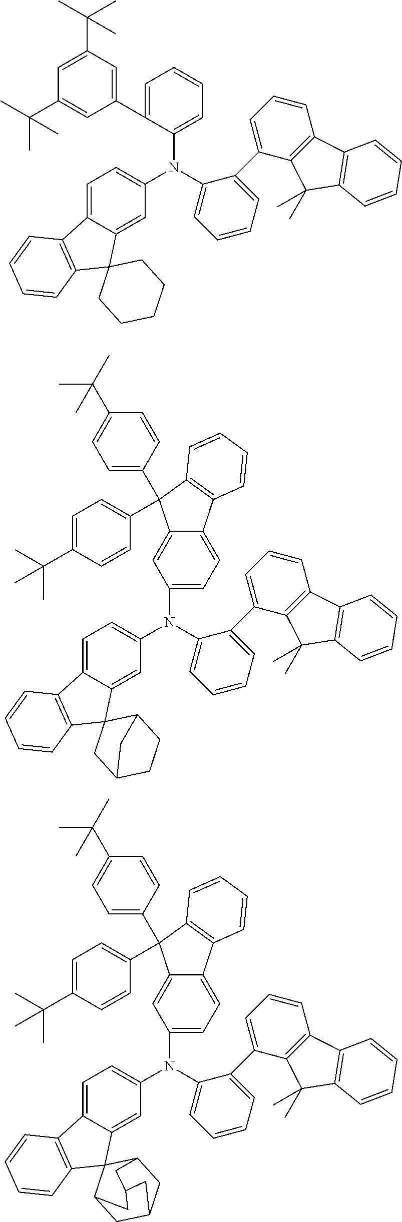

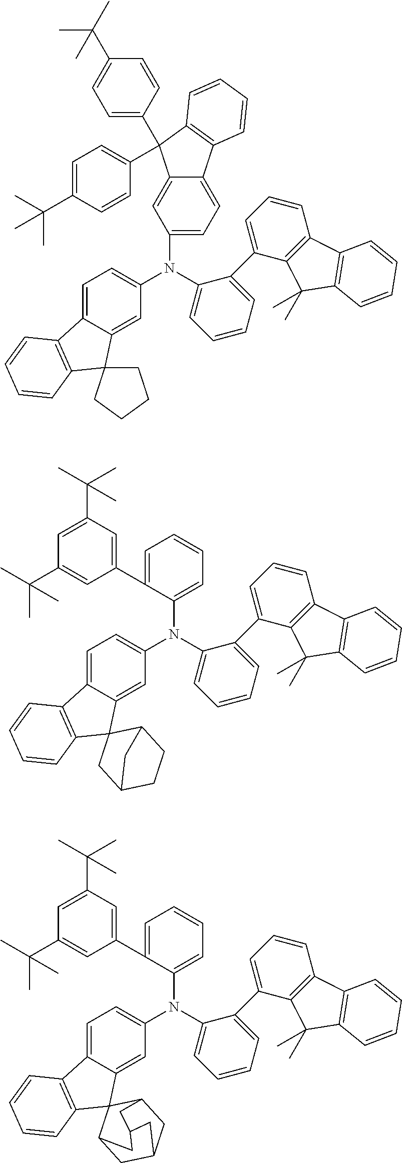 [SMILES: CC(C)(C)c1ccc(C2(c3ccc(C(C)(C)C)cc3)c3ccccc3-c3ccc(N(c4ccc5c(c4)C4(CCCC4)c4ccccc4-5)c4ccccc4-c4cccc5c4C(C)(C)c4ccccc4-5)cc32)cc1 ; CC(C)(C)c1cc(-c2ccccc2N(c2ccc3c(c2)C2(CC4CCC2C4)c2ccccc2-3)c2ccccc2-c2cccc3c2C(C)(C)c2ccccc2-3)cc(C(C)(C)C)c1 ; CC(C)(C)c1cc(-c2ccccc2N(c2ccc3c(c2)C2(c4ccccc4-3)C3CC4CC(C3)CC2C4)c2ccccc2-c2cccc3c2C(C)(C)c2ccccc2-3)cc(C(C)(C)C)c1]

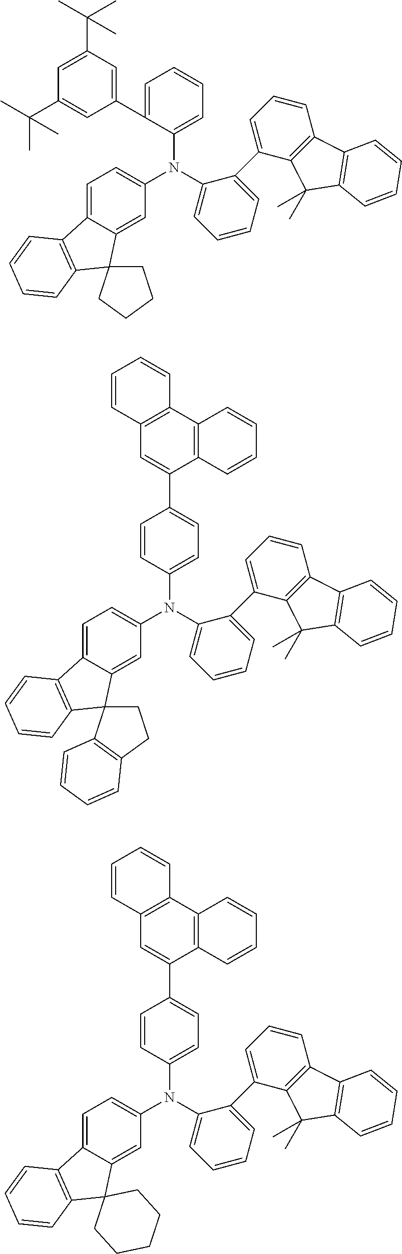

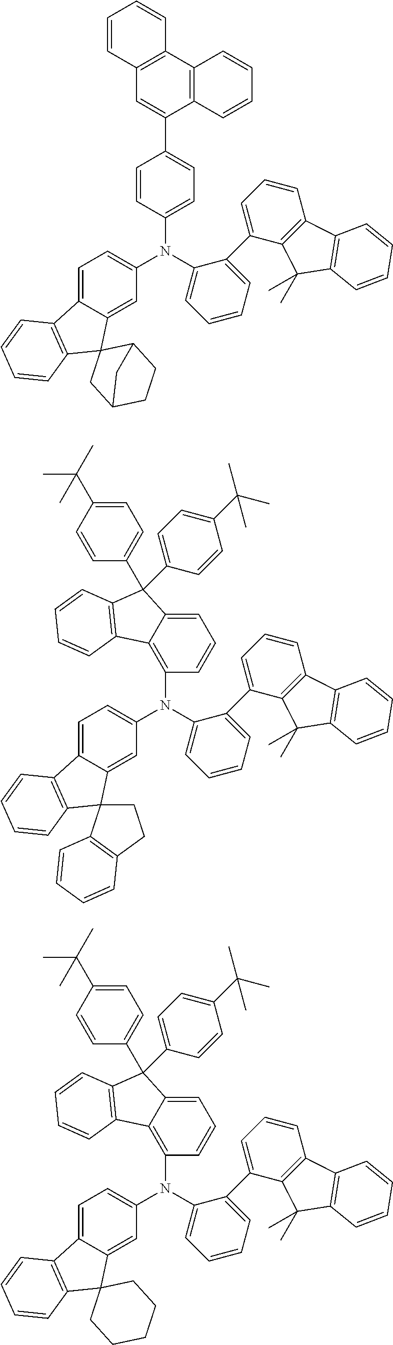

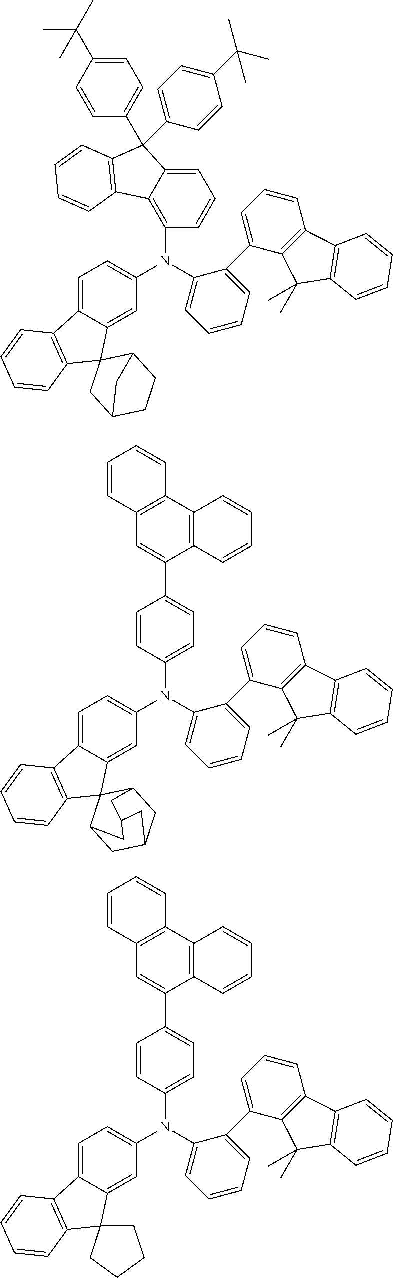

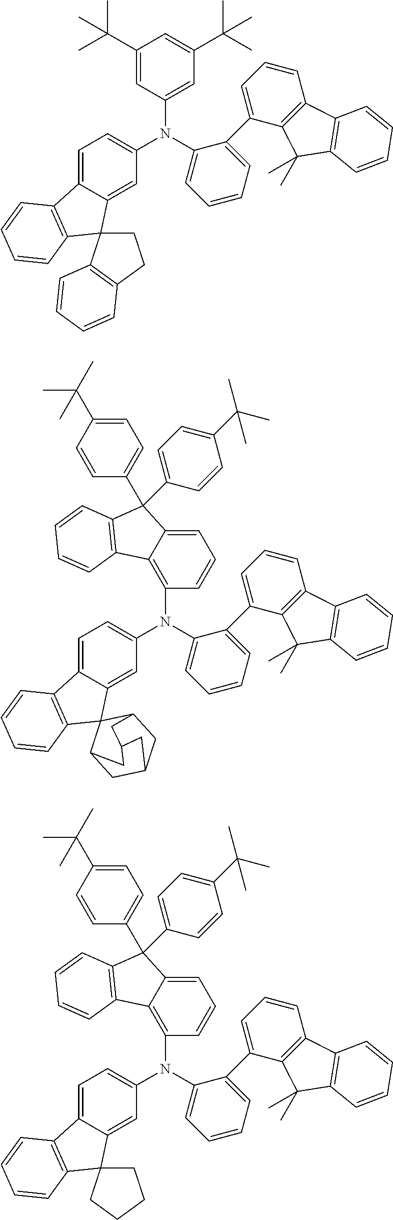

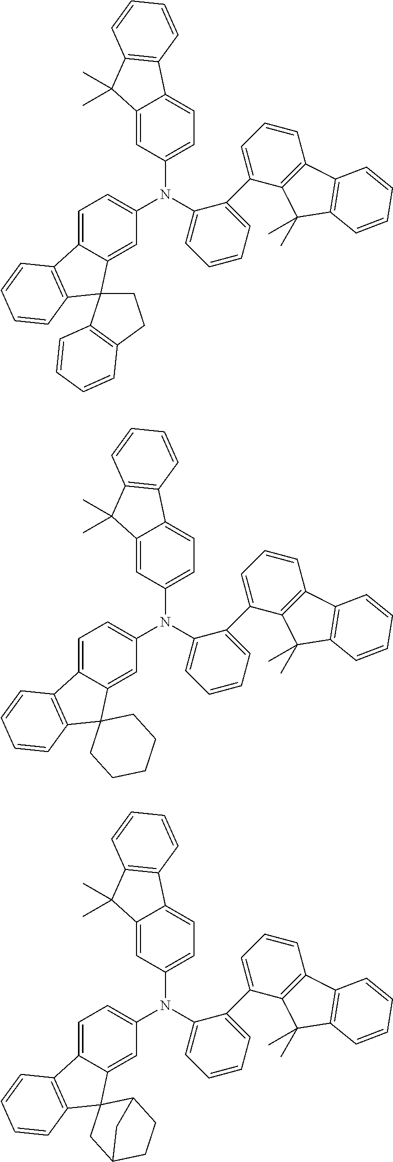

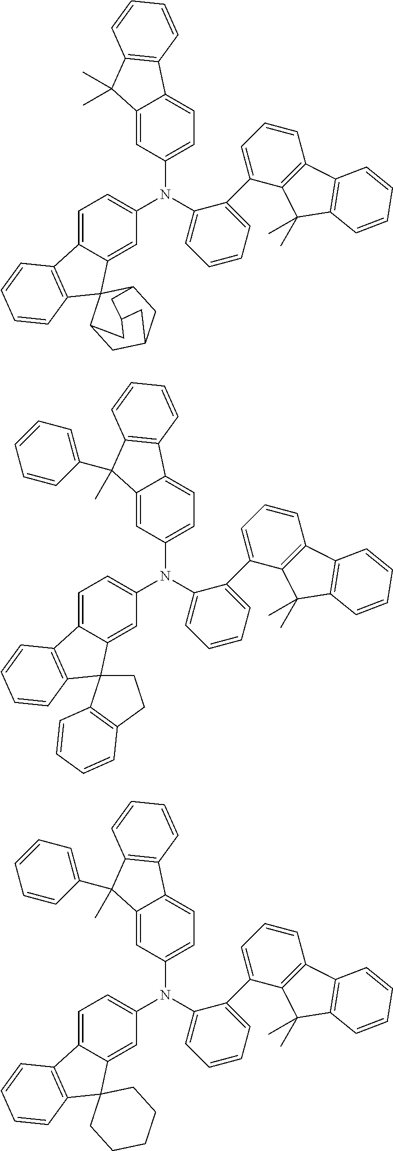

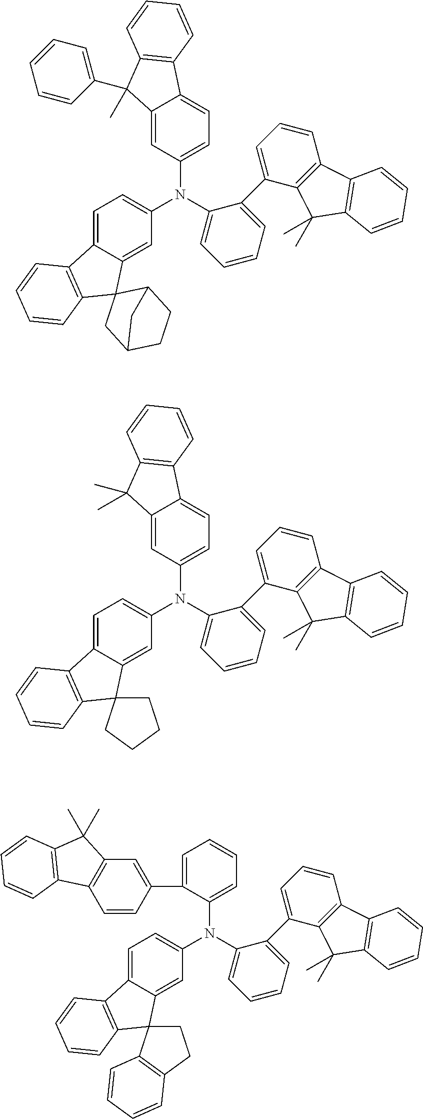 [SMILES: CC1(C)c2ccccc2-c2cccc(-c3ccccc3N(c3ccc4c(c3)C(C)(c3ccccc3)c3ccccc3-4)c3ccc4c(c3)C3(CC5CCC3C5)c3ccccc3-4)c21 ; CC1(C)c2ccccc2-c2ccc(N(c3ccc4c(c3)C3(CCCC3)c3ccccc3-4)c3ccccc3-c3cccc4c3C(C)(C)c3ccccc3-4)cc21 ; CC1(C)c2ccccc2-c2ccc(-c3ccccc3N(c3ccc4c(c3)C3(CCc5ccccc53)c3ccccc3-4)c3ccccc3-c3cccc4c3C(C)(C)c3ccccc3-4)cc21]

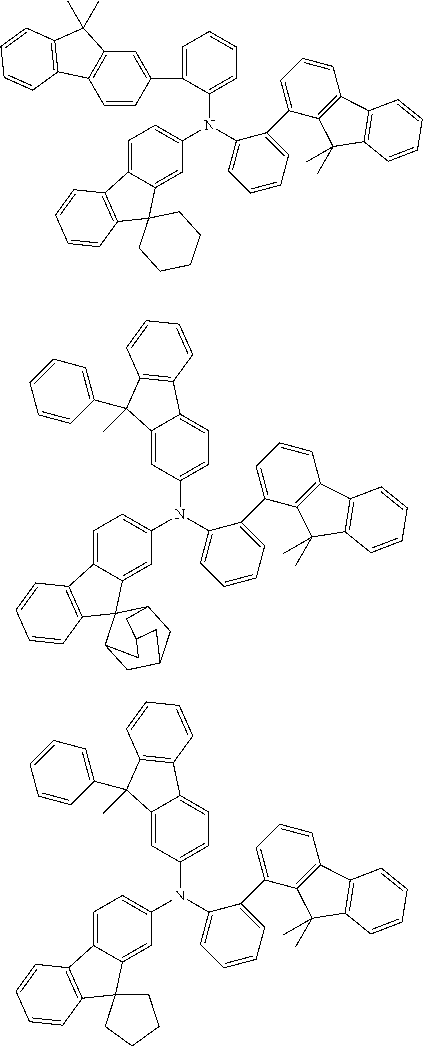

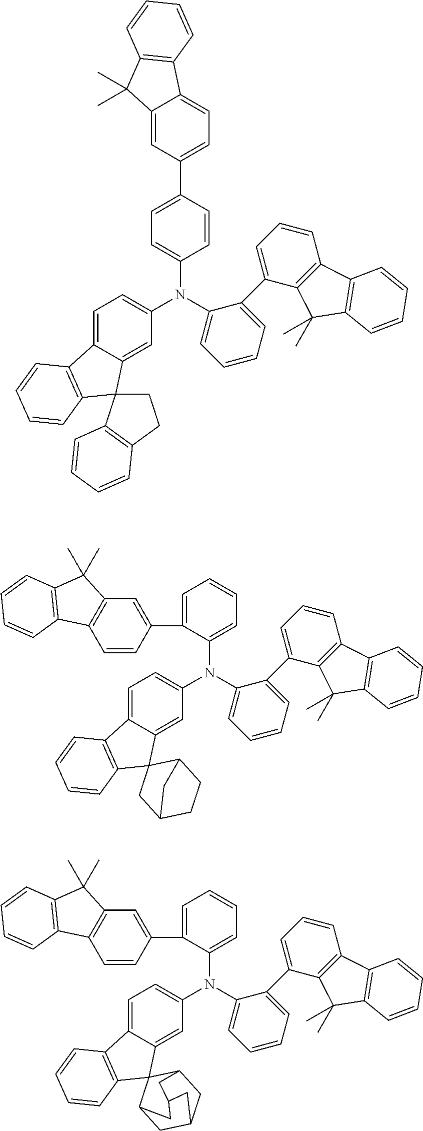 [SMILES: CC1(C)c2ccccc2-c2ccc(-c3ccc(N(c4ccc5c(c4)C4(CCc6ccccc64)c4ccccc4-5)c4ccccc4-c4cccc5c4C(C)(C)c4ccccc4-5)cc3)cc21 ; CC1(C)c2ccccc2-c2ccc(-c3ccccc3N(c3ccc4c(c3)C3(CC5CCC3C5)c3ccccc3-4)c3ccccc3-c3cccc4c3C(C)(C)c3ccccc3-4)cc21 ; CC1(C)c2ccccc2-c2ccc(-c3ccccc3N(c3ccc4c(c3)C3(c5ccccc5-4)C4CC5CC(C4)CC3C5)c3ccccc3-c3cccc4c3C(C)(C)c3ccccc3-4)cc21]

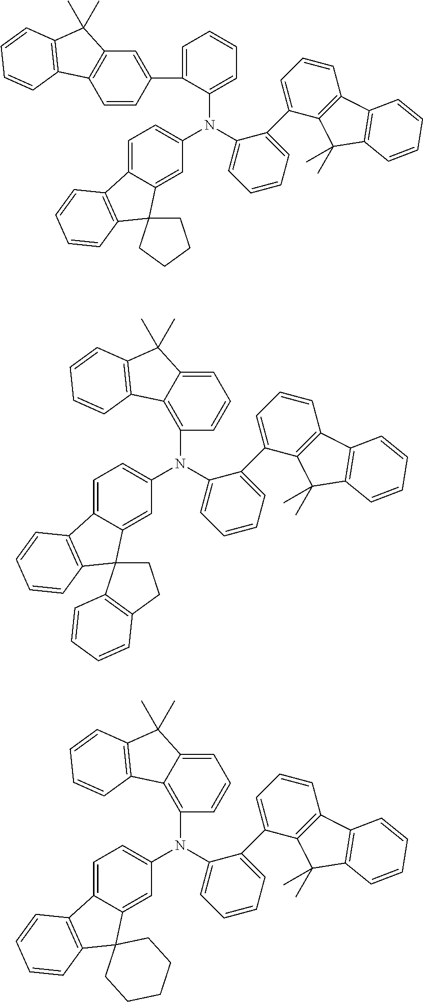 [SMILES: CC1(C)c2ccccc2-c2ccc(-c3ccccc3N(c3ccc4c(c3)C3(CCCC3)c3ccccc3-4)c3ccccc3-c3cccc4c3C(C)(C)c3ccccc3-4)cc21 ; CC1(C)c2ccccc2-c2c(N(c3ccc4c(c3)C3(CCc5ccccc53)c3ccccc3-4)c3ccccc3-c3cccc4c3C(C)(C)c3ccccc3-4)cccc21 ; CC1(C)c2ccccc2-c2c(N(c3ccc4c(c3)C3(CCCCC3)c3ccccc3-4)c3ccccc3-c3cccc4c3C(C)(C)c3ccccc3-4)cccc21]

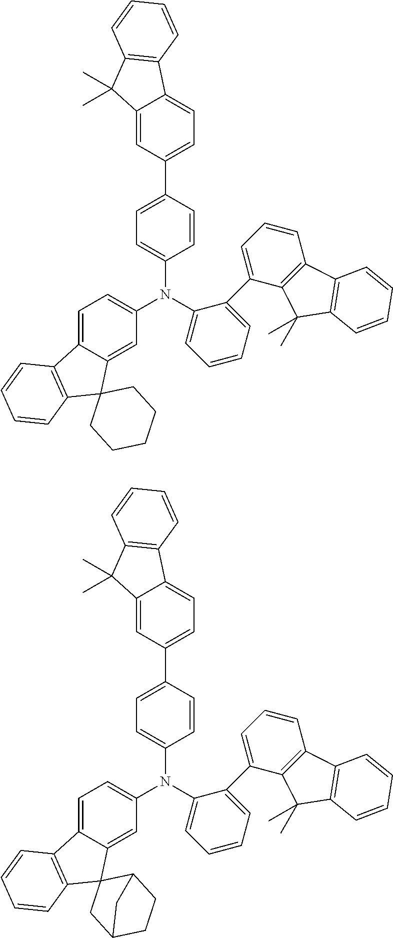

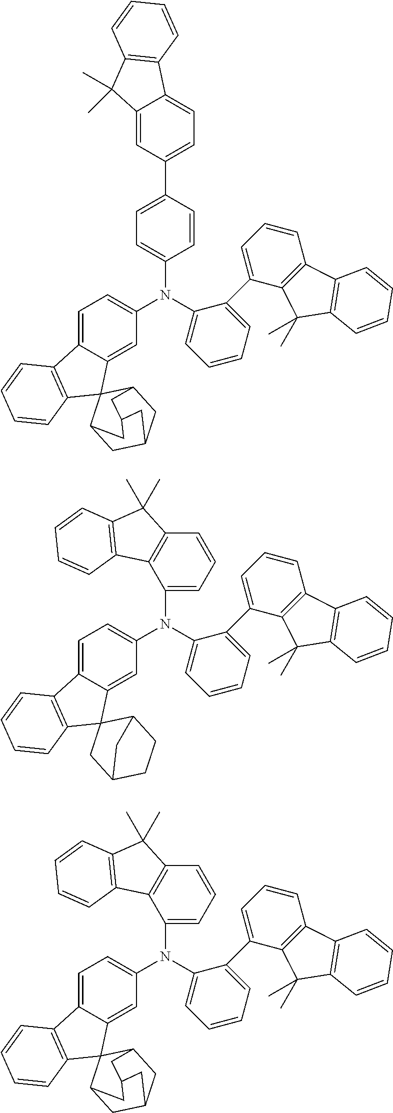

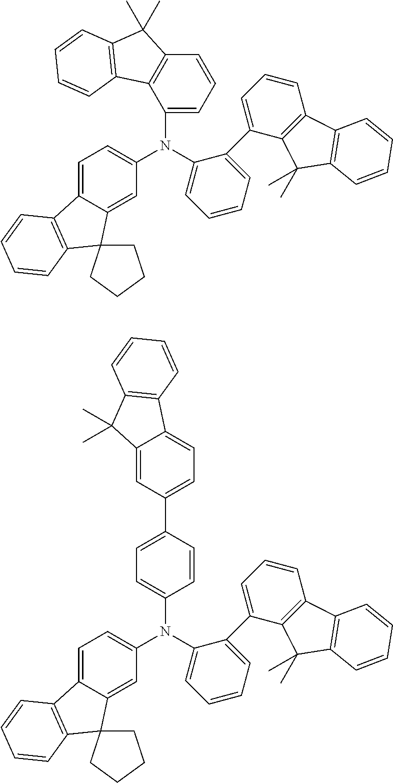

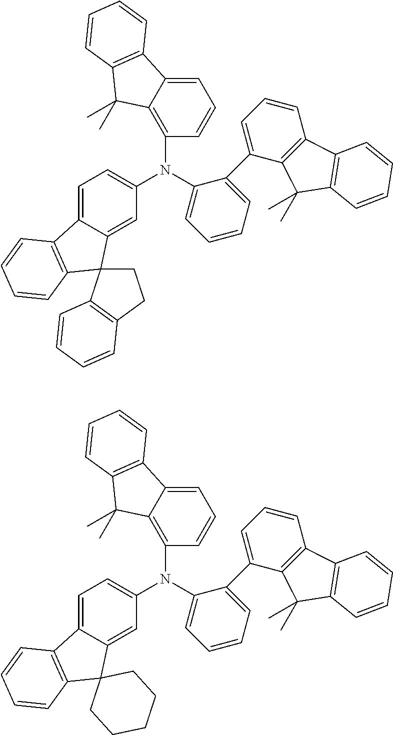 [SMILES: CC1(C)c2ccccc2-c2cccc(-c3ccccc3N(c3ccc4c(c3)C3(CCc5ccccc53)c3ccccc3-4)c3cccc4c3C(C)(C)c3ccccc3-4)c21 ; CC1(C)c2ccccc2-c2cccc(-c3ccccc3N(c3ccc4c(c3)C3(CCCCC3)c3ccccc3-4)c3cccc4c3C(C)(C)c3ccccc3-4)c21]

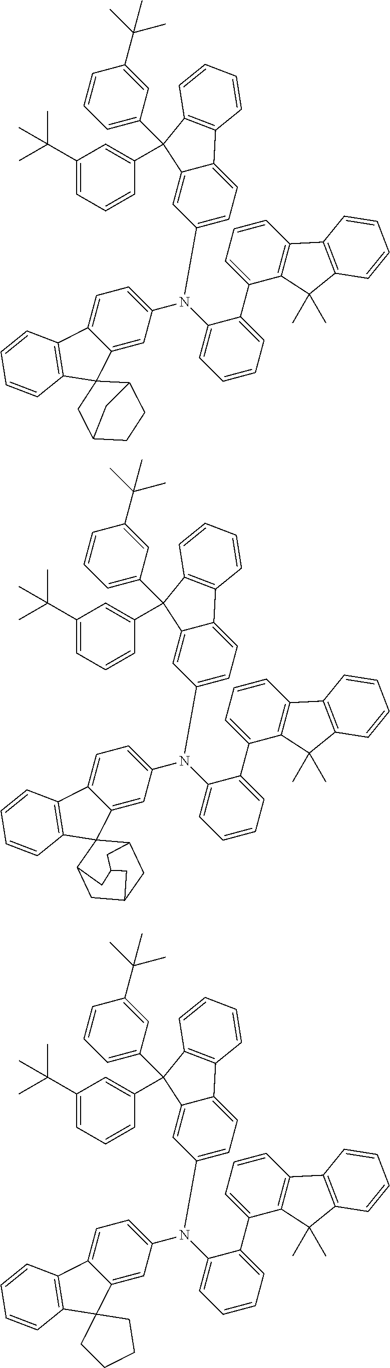

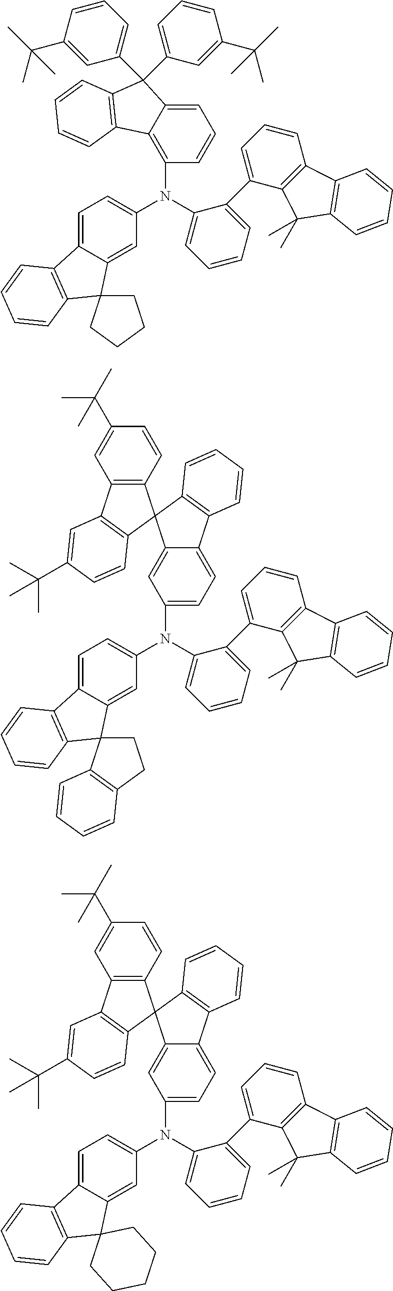

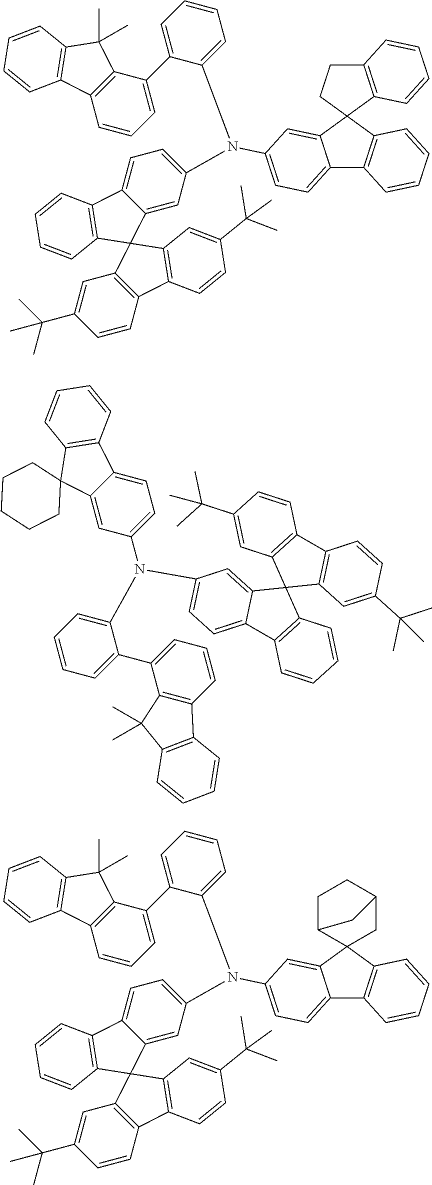

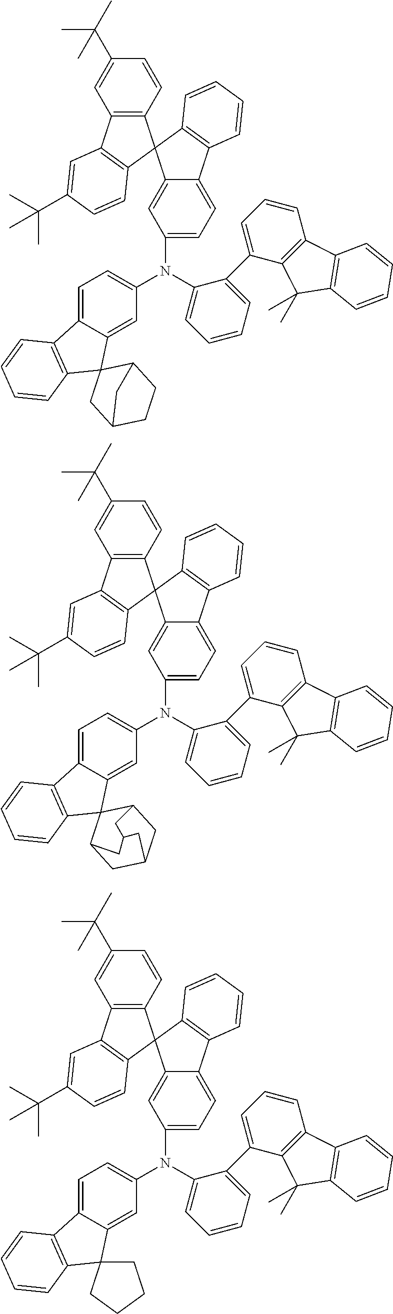

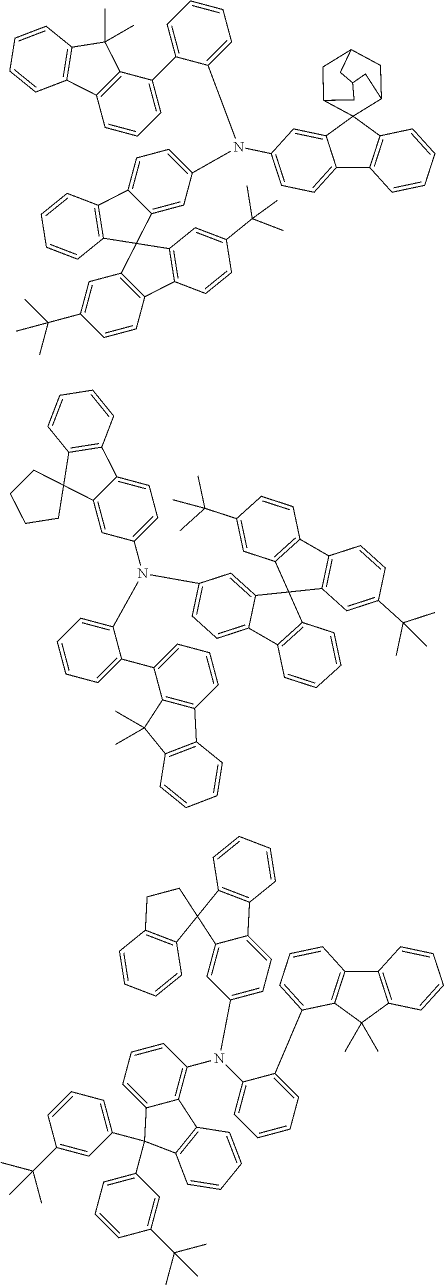

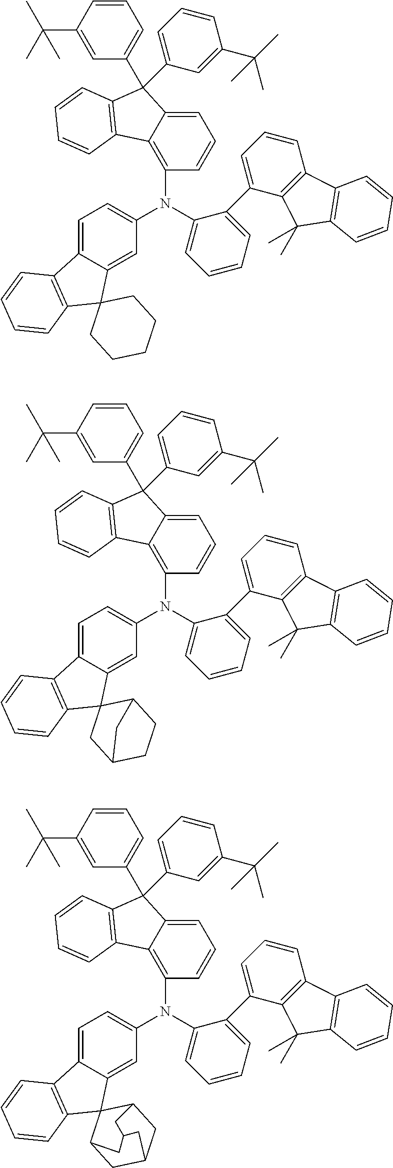 [SMILES: CC(C)(C)c1cccc(C2(c3cccc(C(C)(C)C)c3)c3ccccc3-c3c(N(c4ccc5c(c4)C4(CCCCC4)c4ccccc4-5)c4ccccc4-c4cccc5c4C(C)(C)c4ccccc4-5)cccc32)c1 ; CC(C)(C)c1cccc(C2(c3cccc(C(C)(C)C)c3)c3ccccc3-c3c(N(c4ccc5c(c4)C4(CC6CCC4C6)c4ccccc4-5)c4ccccc4-c4cccc5c4C(C)(C)c4ccccc4-5)cccc32)c1 ; CC(C)(C)c1cccc(C2(c3cccc(C(C)(C)C)c3)c3ccccc3-c3c(N(c4ccc5c(c4)C4(c6ccccc6-5)C5CC6CC(C5)CC4C6)c4ccccc4-c4cccc5c4C(C)(C)c4ccccc4-5)cccc32)c1]

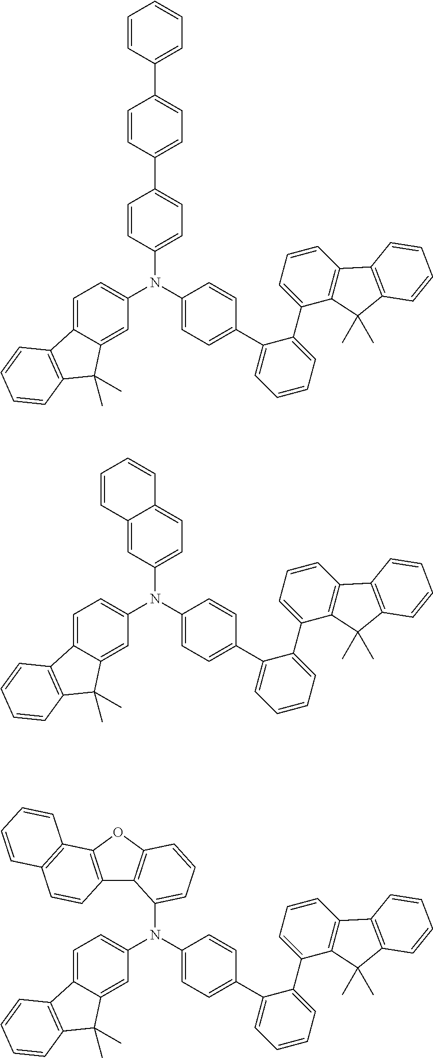 [SMILES: CC1(C)c2ccccc2-c2ccc(N(c3ccc(-c4ccc(-c5ccccc5)cc4)cc3)c3ccc(-c4ccccc4-c4cccc5c4C(C)(C)c4ccccc4-5)cc3)cc21 ; CC1(C)c2ccccc2-c2ccc(N(c3ccc(-c4ccccc4-c4cccc5c4C(C)(C)c4ccccc4-5)cc3)c3ccc4ccccc4c3)cc21 ; CC1(C)c2ccccc2-c2ccc(N(c3ccc(-c4ccccc4-c4cccc5c4C(C)(C)c4ccccc4-5)cc3)c3cccc4oc5c6ccccc6ccc5c34)cc21]

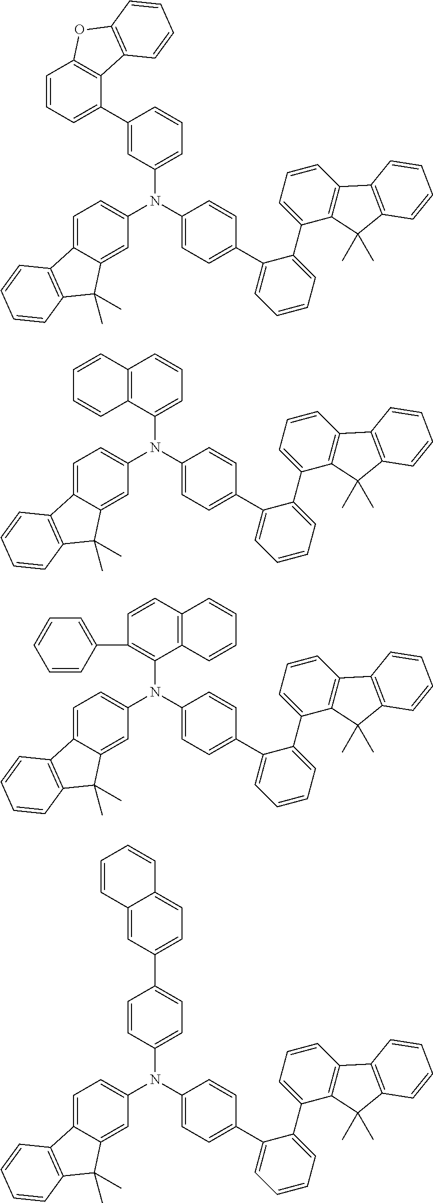

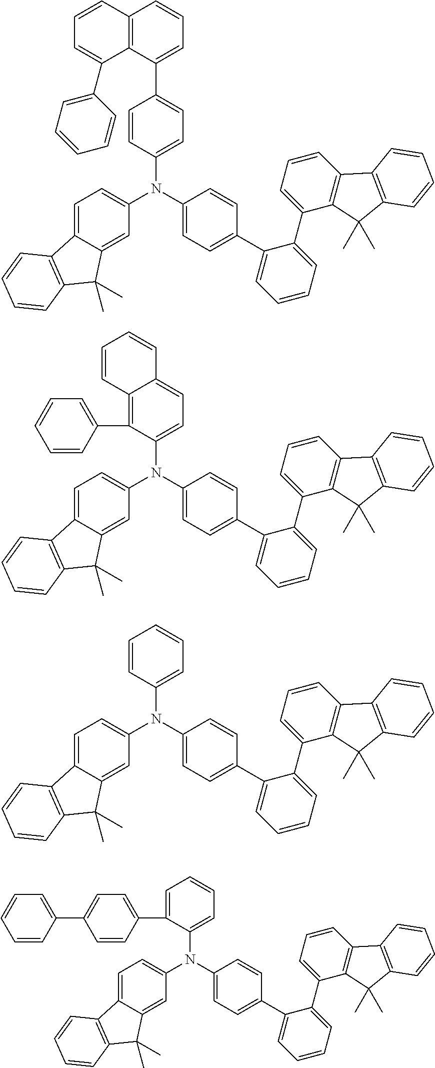

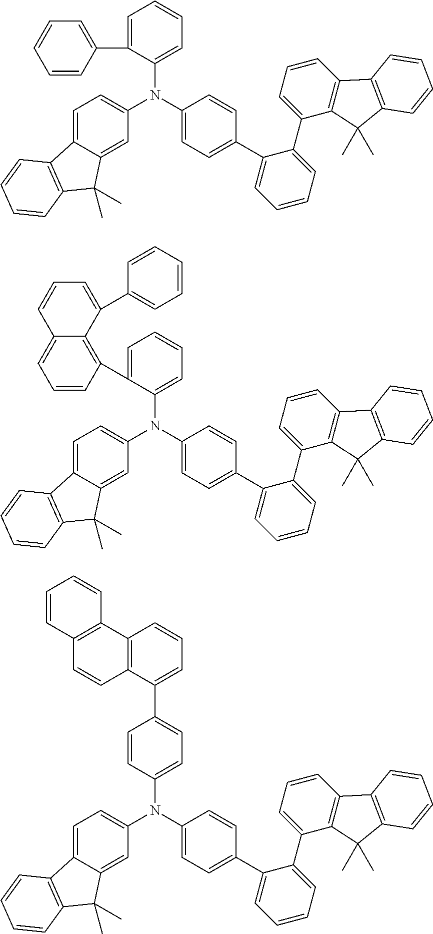

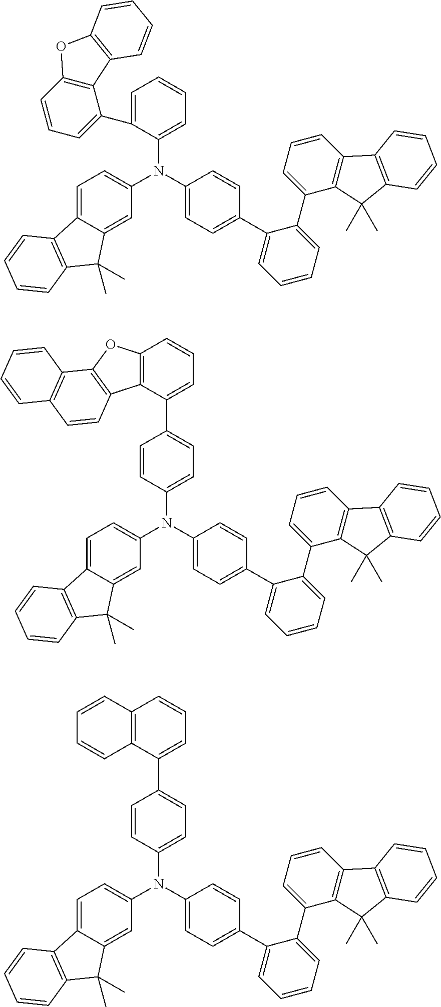 [SMILES: CC1(C)c2ccccc2-c2ccc(N(c3ccc(-c4ccccc4-c4cccc5c4C(C)(C)c4ccccc4-5)cc3)c3ccccc3-c3cccc4oc5ccccc5c34)cc21 ; CC1(C)c2ccccc2-c2ccc(N(c3ccc(-c4ccccc4-c4cccc5c4C(C)(C)c4ccccc4-5)cc3)c3ccc(-c4cccc5oc6c7ccccc7ccc6c45)cc3)cc21 ; CC1(C)c2ccccc2-c2ccc(N(c3ccc(-c4ccccc4-c4cccc5c4C(C)(C)c4ccccc4-5)cc3)c3ccc(-c4cccc5ccccc45)cc3)cc21]

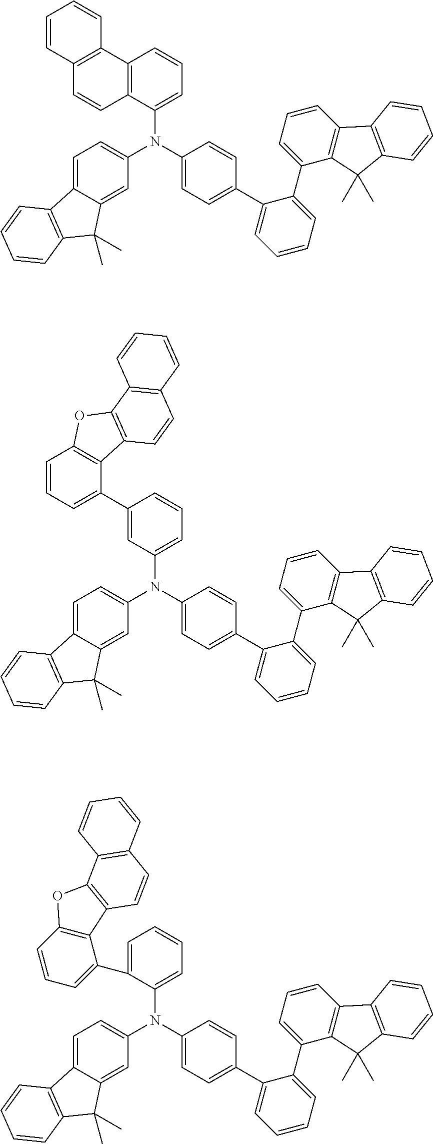 [SMILES: CC1(C)c2ccccc2-c2ccc(N(c3ccc(-c4ccccc4-c4cccc5c4C(C)(C)c4ccccc4-5)cc3)c3cccc4c3ccc3ccccc34)cc21 ; CC1(C)c2ccccc2-c2ccc(N(c3ccc(-c4ccccc4-c4cccc5c4C(C)(C)c4ccccc4-5)cc3)c3cccc(-c4cccc5oc6c7ccccc7ccc6c45)c3)cc21 ; CC1(C)c2ccccc2-c2ccc(N(c3ccc(-c4ccccc4-c4cccc5c4C(C)(C)c4ccccc4-5)cc3)c3ccccc3-c3cccc4oc5c6ccccc6ccc5c34)cc21]

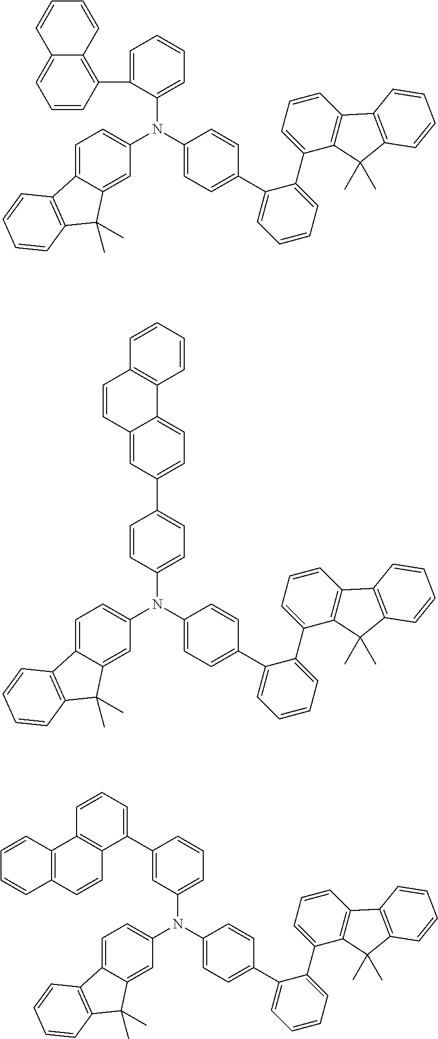 [SMILES: CC1(C)c2ccccc2-c2ccc(N(c3ccc(-c4ccccc4-c4cccc5c4C(C)(C)c4ccccc4-5)cc3)c3ccccc3-c3cccc4ccccc34)cc21 ; CC1(C)c2ccccc2-c2ccc(N(c3ccc(-c4ccc5c(ccc6ccccc65)c4)cc3)c3ccc(-c4ccccc4-c4cccc5c4C(C)(C)c4ccccc4-5)cc3)cc21 ; CC1(C)c2ccccc2-c2ccc(N(c3ccc(-c4ccccc4-c4cccc5c4C(C)(C)c4ccccc4-5)cc3)c3cccc(-c4cccc5c4ccc4ccccc45)c3)cc21]

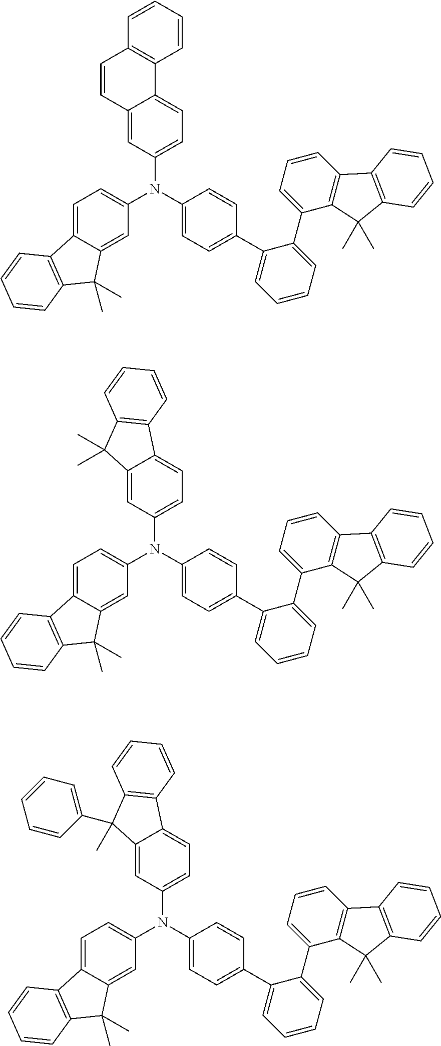 [SMILES: CC1(C)c2ccccc2-c2ccc(N(c3ccc(-c4ccccc4-c4cccc5c4C(C)(C)c4ccccc4-5)cc3)c3ccc4c(ccc5ccccc54)c3)cc21 ; CC1(C)c2ccccc2-c2ccc(N(c3ccc(-c4ccccc4-c4cccc5c4C(C)(C)c4ccccc4-5)cc3)c3ccc4c(c3)C(C)(C)c3ccccc3-4)cc21 ; CC1(C)c2ccccc2-c2ccc(N(c3ccc(-c4ccccc4-c4cccc5c4C(C)(C)c4ccccc4-5)cc3)c3ccc4c(c3)C(C)(c3ccccc3)c3ccccc3-4)cc21]

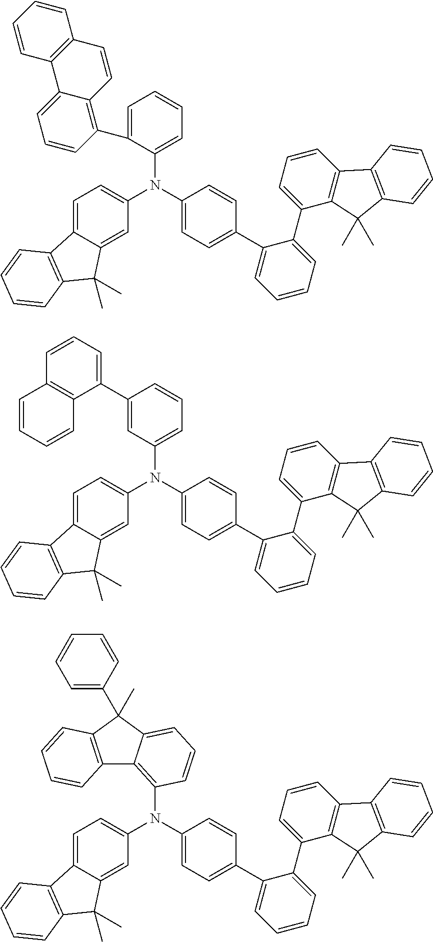

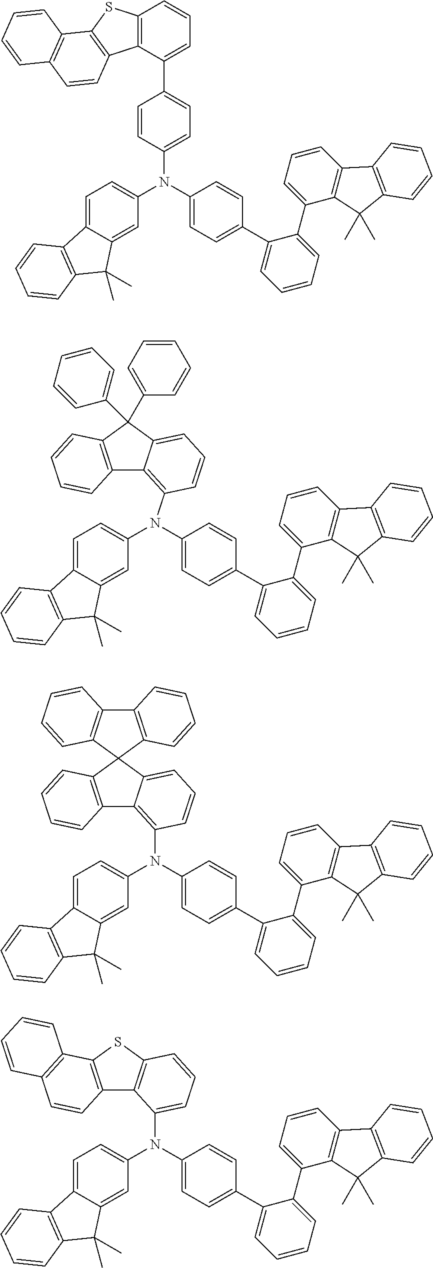

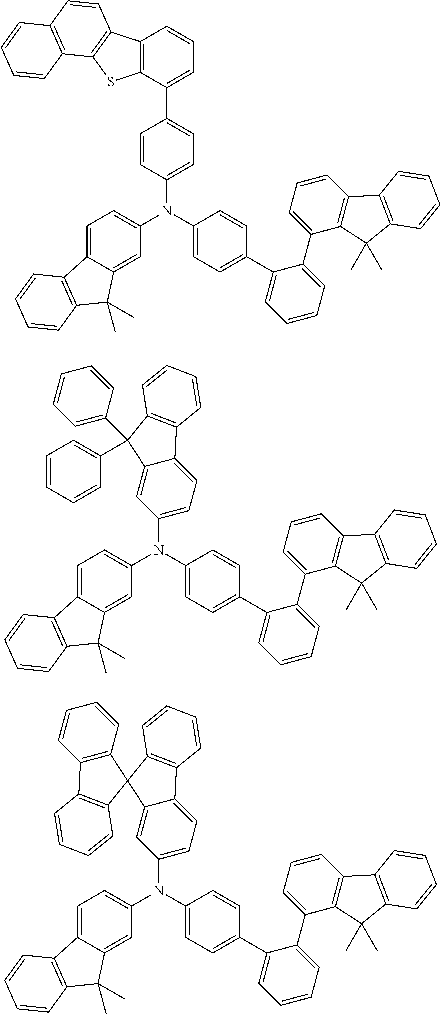

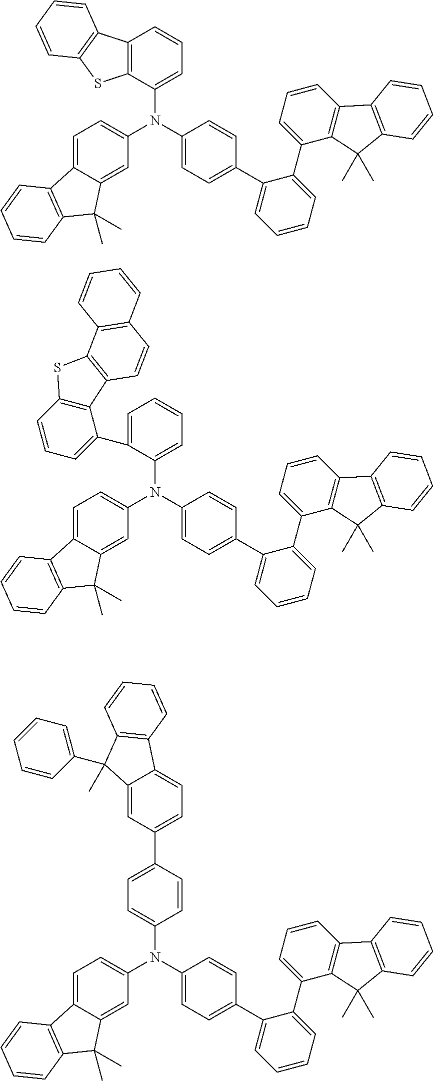

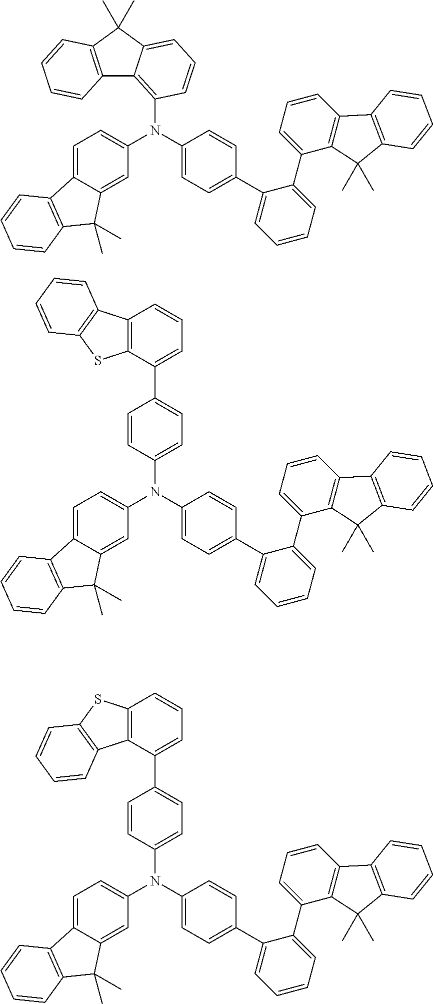

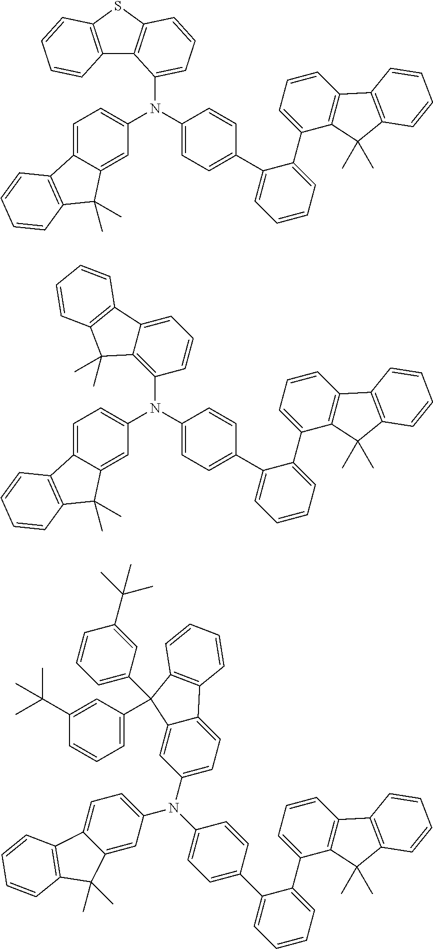

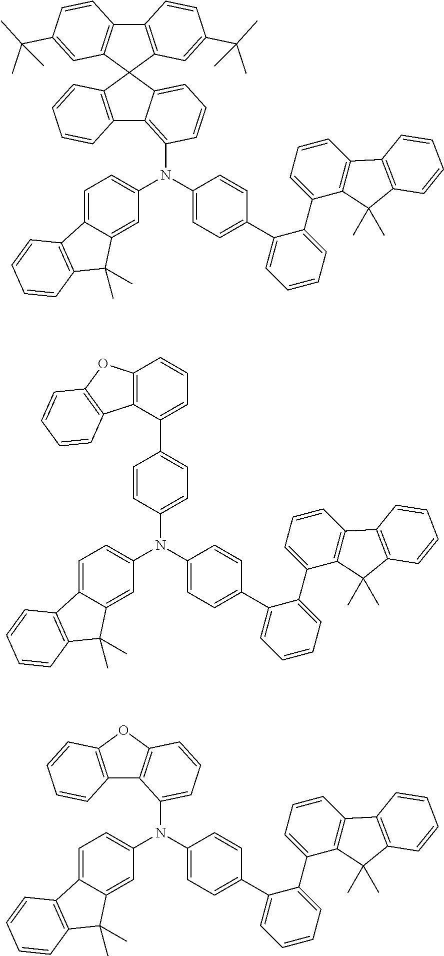 [SMILES: CC(C)(C)c1ccc2c(c1)C1(c3cc(C(C)(C)C)ccc3-2)c2ccccc2-c2c(N(c3ccc(-c4ccccc4-c4cccc5c4C(C)(C)c4ccccc4-5)cc3)c3ccc4c(c3)C(C)(C)c3ccccc3-4)cccc21 ; CC1(C)c2ccccc2-c2ccc(N(c3ccc(-c4ccccc4-c4cccc5c4C(C)(C)c4ccccc4-5)cc3)c3ccc(-c4cccc5oc6ccccc6c45)cc3)cc21 ; CC1(C)c2ccccc2-c2ccc(N(c3ccc(-c4ccccc4-c4cccc5c4C(C)(C)c4ccccc4-5)cc3)c3cccc4oc5ccccc5c34)cc21]

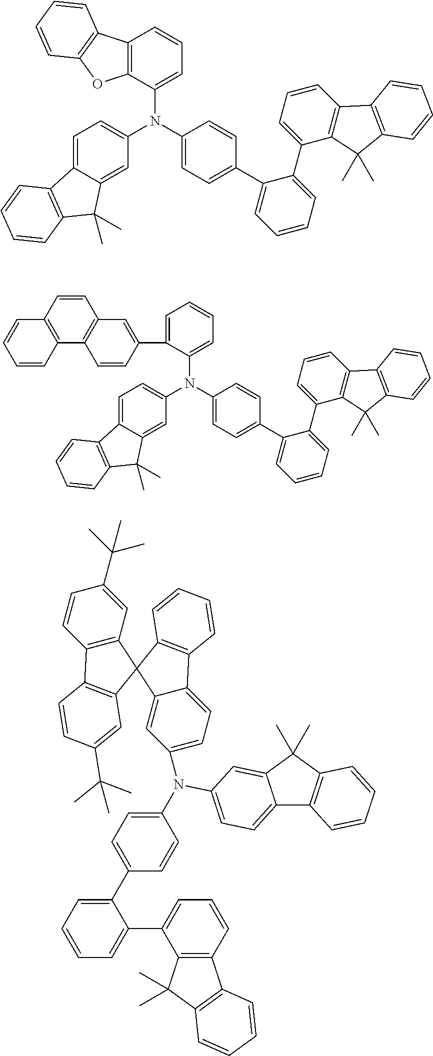

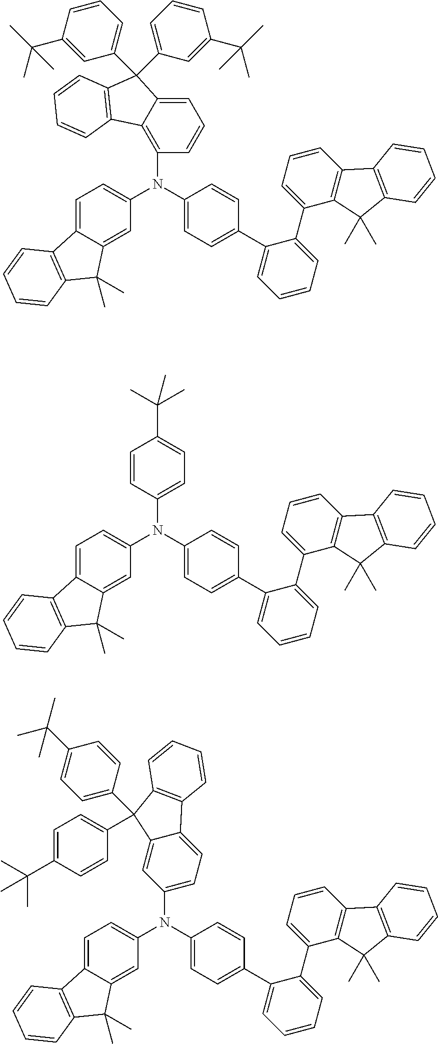 [SMILES: CC(C)(C)c1cccc(C2(c3cccc(C(C)(C)C)c3)c3ccccc3-c3c(N(c4ccc(-c5ccccc5-c5cccc6c5C(C)(C)c5ccccc5-6)cc4)c4ccc5c(c4)C(C)(C)c4ccccc4-5)cccc32)c1 ; CC(C)(C)c1ccc(N(c2ccc(-c3ccccc3-c3cccc4c3C(C)(C)c3ccccc3-4)cc2)c2ccc3c(c2)C(C)(C)c2ccccc2-3)cc1 ; CC(C)(C)c1ccc(C2(c3ccc(C(C)(C)C)cc3)c3ccccc3-c3ccc(N(c4ccc(-c5ccccc5-c5cccc6c5C(C)(C)c5ccccc5-6)cc4)c4ccc5c(c4)C(C)(C)c4ccccc4-5)cc32)cc1]

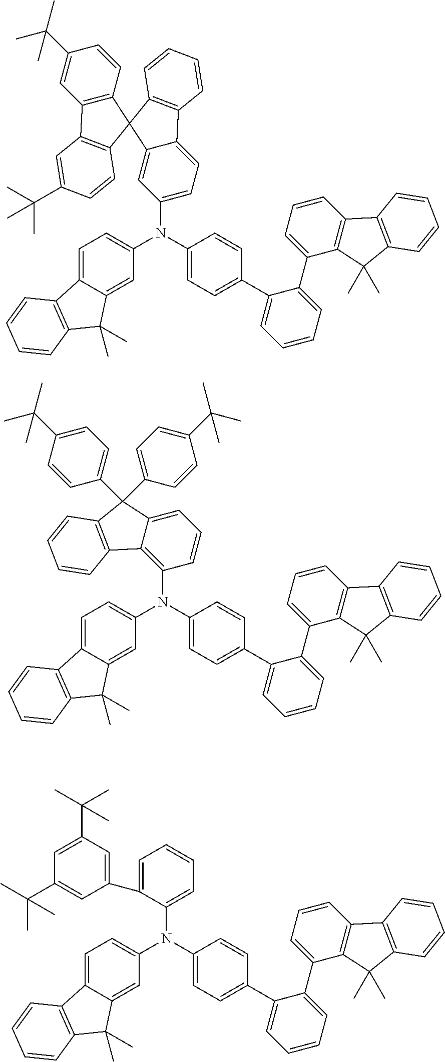

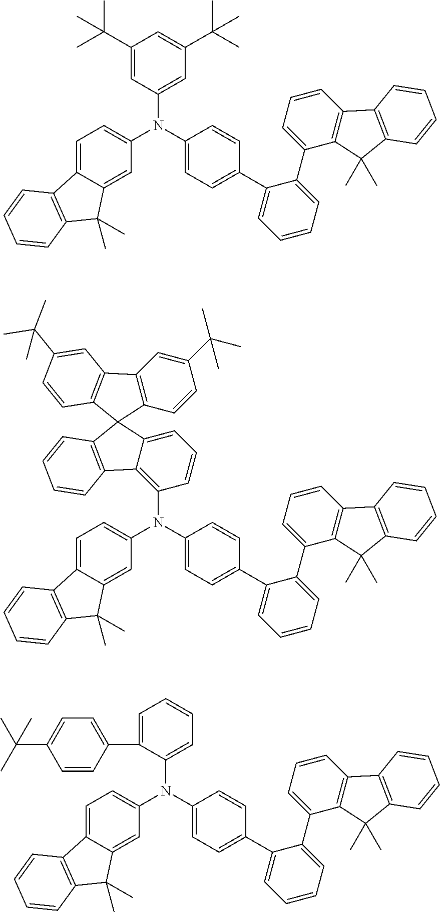 [SMILES: CC(C)(C)c1cc(N(c2ccc(-c3ccccc3-c3cccc4c3C(C)(C)c3ccccc3-4)cc2)c2ccc3c(c2)C(C)(C)c2ccccc2-3)cc(C(C)(C)C)c1 ; CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C(C)(C)c3ccccc3-4)cccc21 ; CC(C)(C)c1ccc(-c2ccccc2N(c2ccc(-c3ccccc3-c3cccc4c3C(C)(C)c3ccccc3-4)cc2)c2ccc3c(c2)C(C)(C)c2ccccc2-3)cc1]

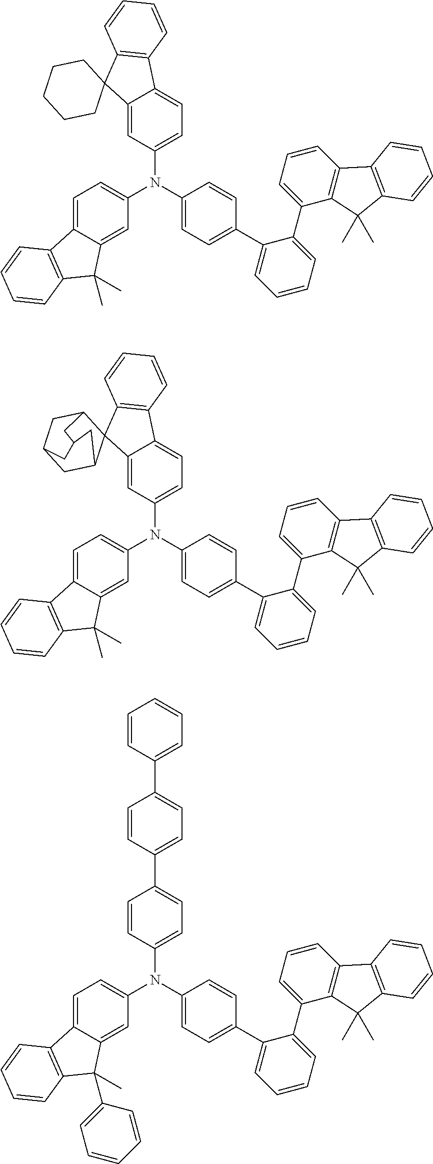 [SMILES: CC1(C)c2ccccc2-c2ccc(N(c3ccc(-c4ccccc4-c4cccc5c4C(C)(C)c4ccccc4-5)cc3)c3ccc4c(c3)C3(CCCCC3)c3ccccc3-4)cc21 ; CC1(C)c2ccccc2-c2ccc(N(c3ccc(-c4ccccc4-c4cccc5c4C(C)(C)c4ccccc4-5)cc3)c3ccc4c(c3)C3(c5ccccc5-4)C4CC5CC(C4)CC3C5)cc21 ; CC1(C)c2ccccc2-c2cccc(-c3ccccc3-c3ccc(N(c4ccc(-c5ccc(-c6ccccc6)cc5)cc4)c4ccc5c(c4)C(C)(c4ccccc4)c4ccccc4-5)cc3)c21]

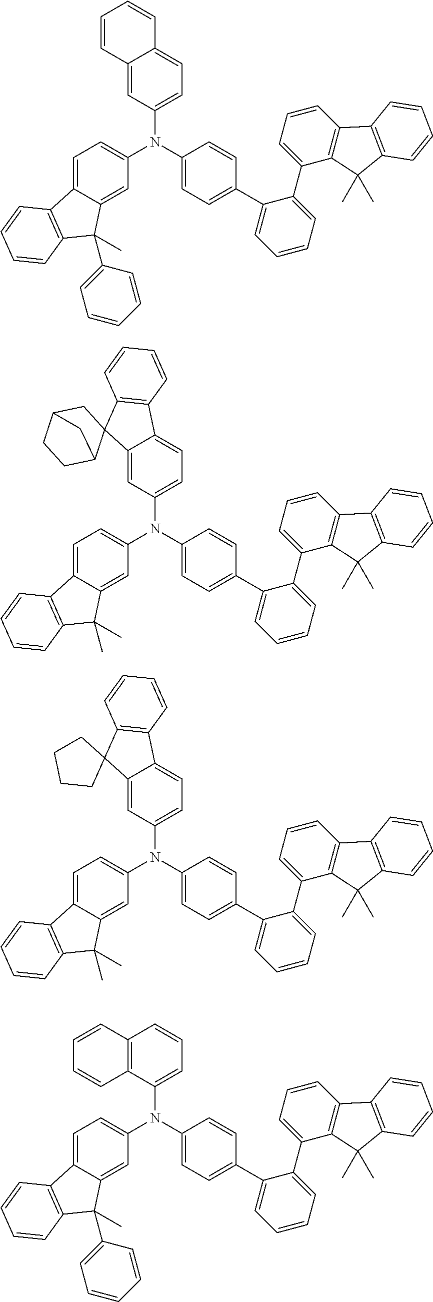

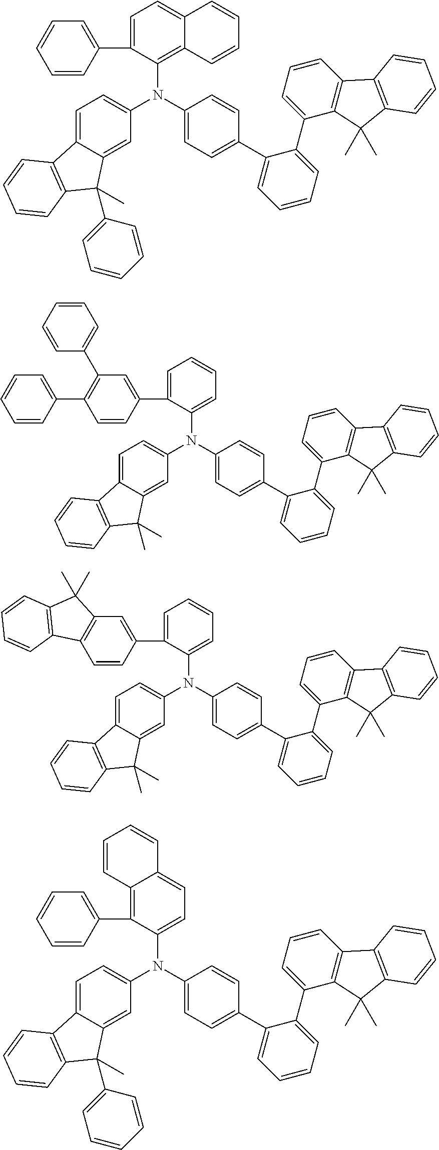

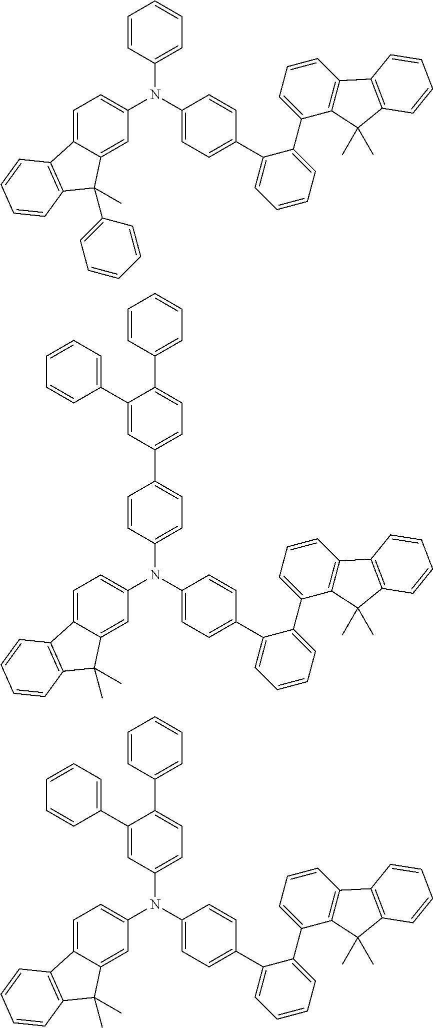

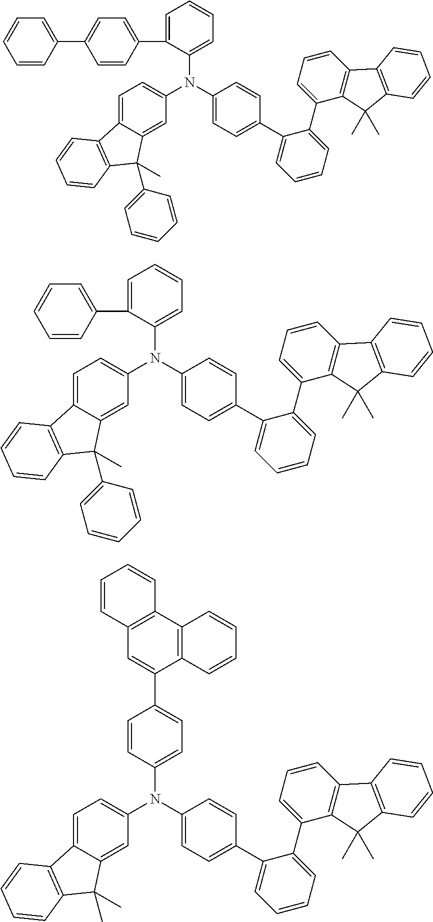

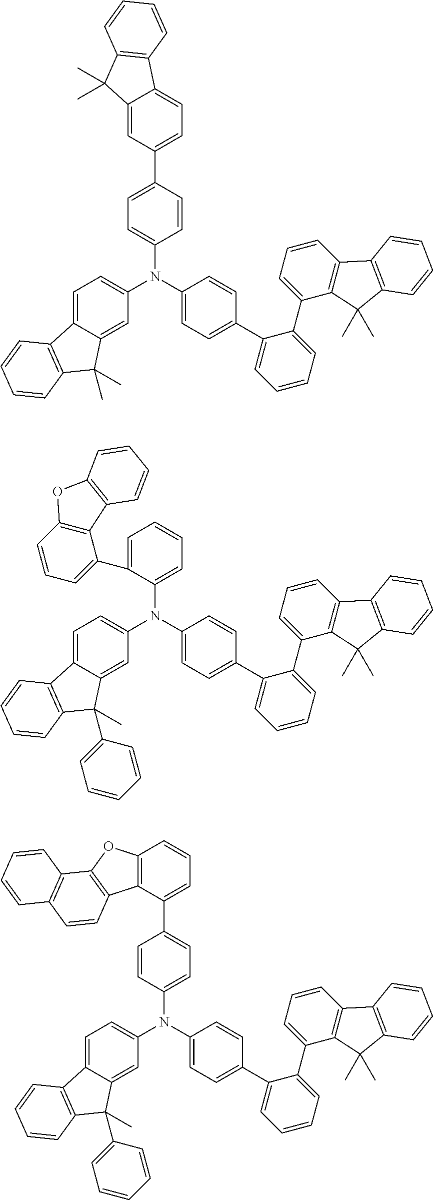 [SMILES: CC1(C)c2ccccc2-c2ccc(-c3ccc(N(c4ccc(-c5ccccc5-c5cccc6c5C(C)(C)c5ccccc5-6)cc4)c4ccc5c(c4)C(C)(C)c4ccccc4-5)cc3)cc21 ; CC1(C)c2ccccc2-c2cccc(-c3ccccc3-c3ccc(N(c4ccc5c(c4)C(C)(c4ccccc4)c4ccccc4-5)c4ccccc4-c4cccc5oc6ccccc6c45)cc3)c21 ; CC1(C)c2ccccc2-c2cccc(-c3ccccc3-c3ccc(N(c4ccc(-c5cccc6oc7c8ccccc8ccc7c56)cc4)c4ccc5c(c4)C(C)(c4ccccc4)c4ccccc4-5)cc3)c21]

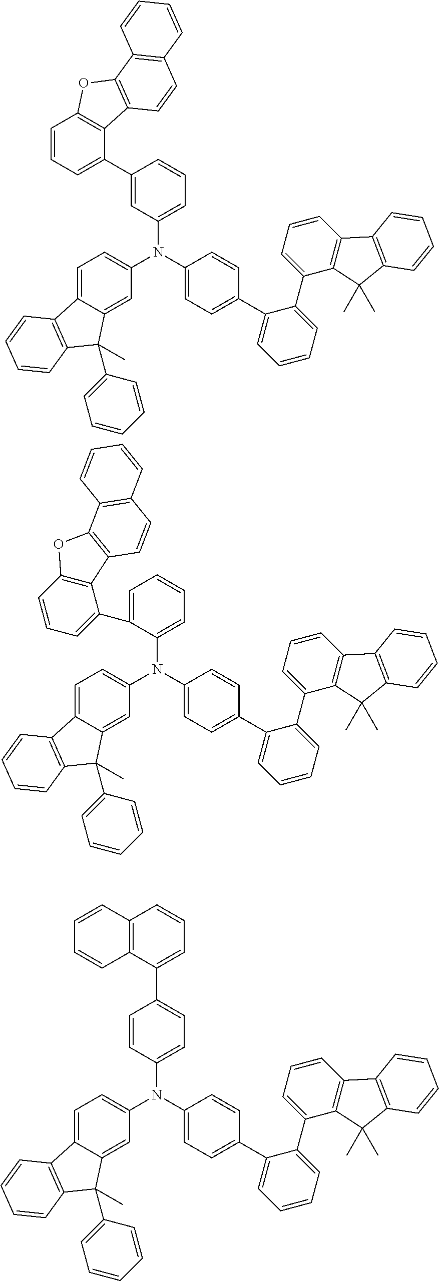

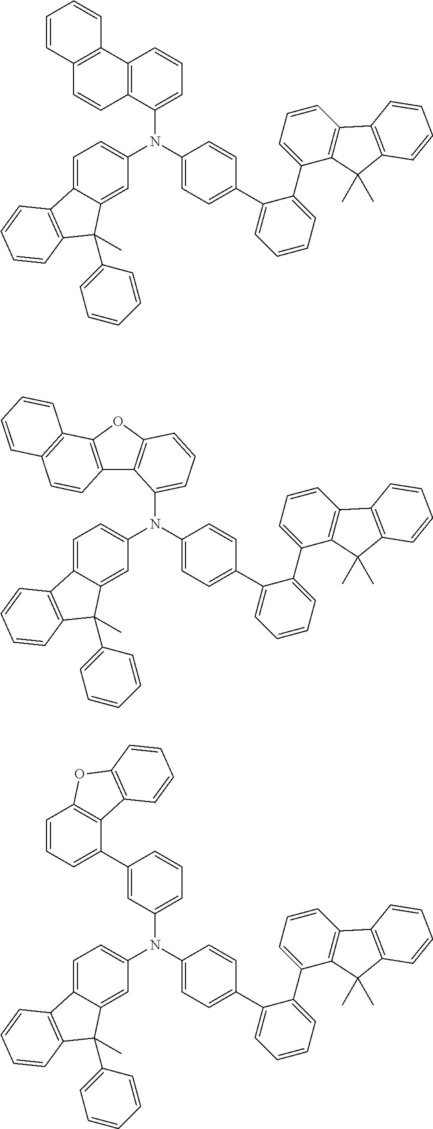

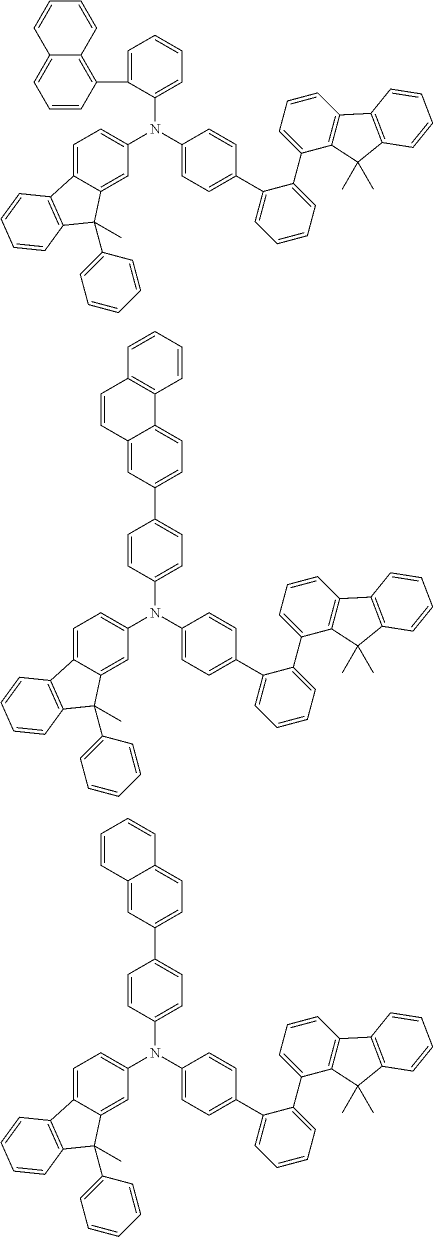

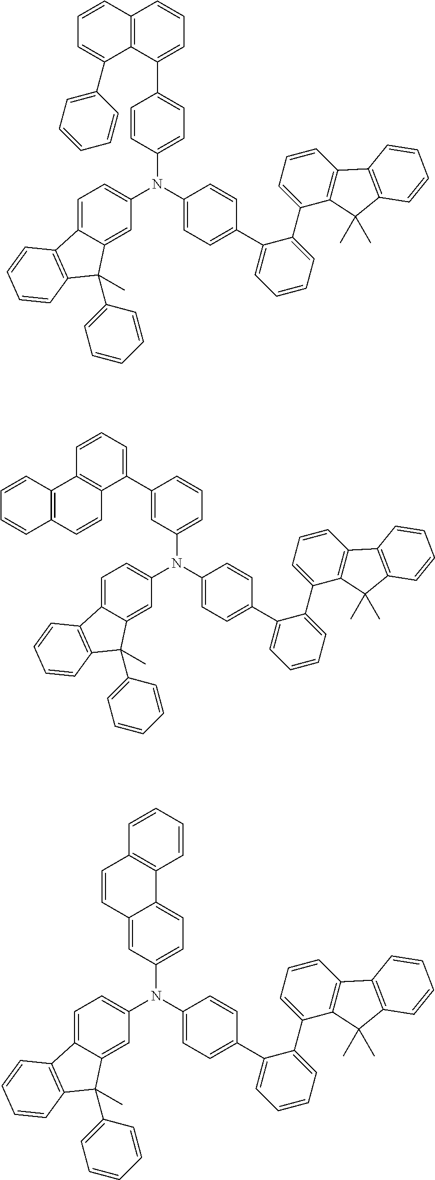 [SMILES: CC1(C)c2ccccc2-c2cccc(-c3ccccc3-c3ccc(N(c4ccc(-c5cccc6cccc(-c7ccccc7)c56)cc4)c4ccc5c(c4)C(C)(c4ccccc4)c4ccccc4-5)cc3)c21 ; CC1(C)c2ccccc2-c2cccc(-c3ccccc3-c3ccc(N(c4cccc(-c5cccc6c5ccc5ccccc56)c4)c4ccc5c(c4)C(C)(c4ccccc4)c4ccccc4-5)cc3)c21 ; CC1(C)c2ccccc2-c2cccc(-c3ccccc3-c3ccc(N(c4ccc5c(c4)C(C)(c4ccccc4)c4ccccc4-5)c4ccc5c(ccc6ccccc65)c4)cc3)c21]

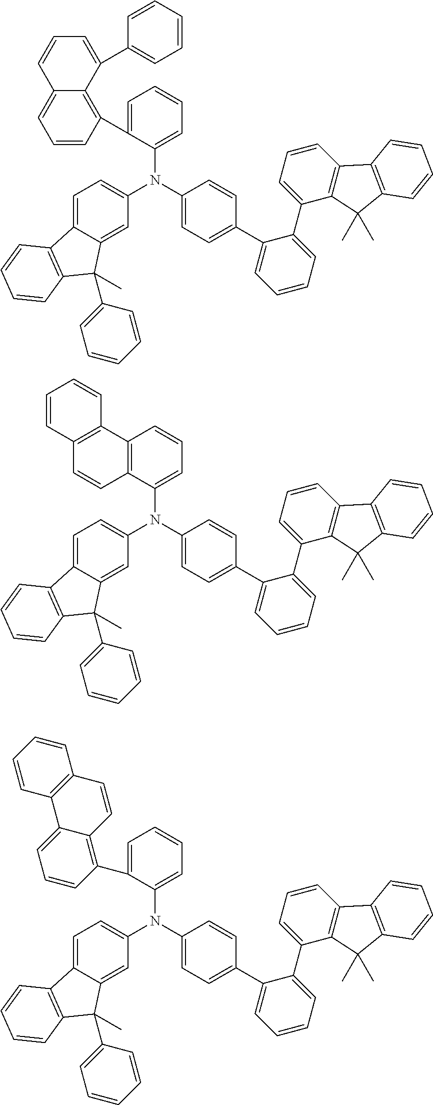

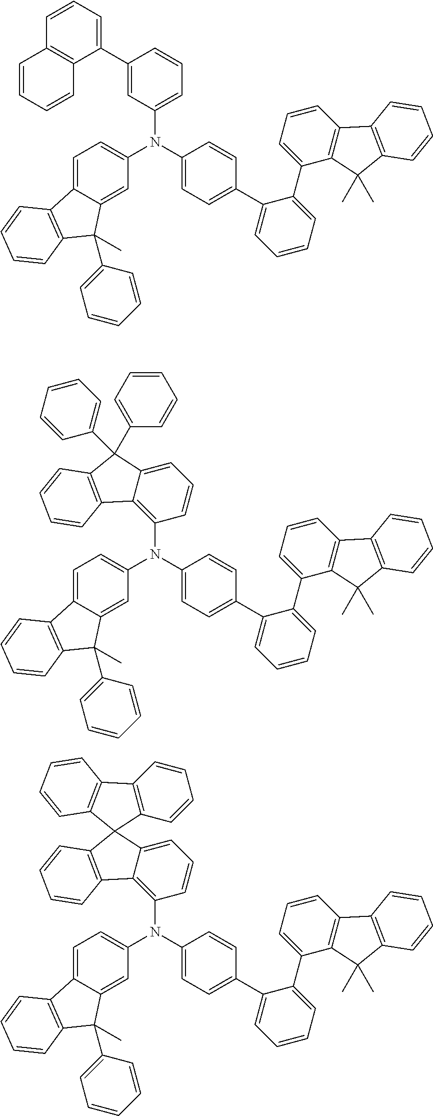

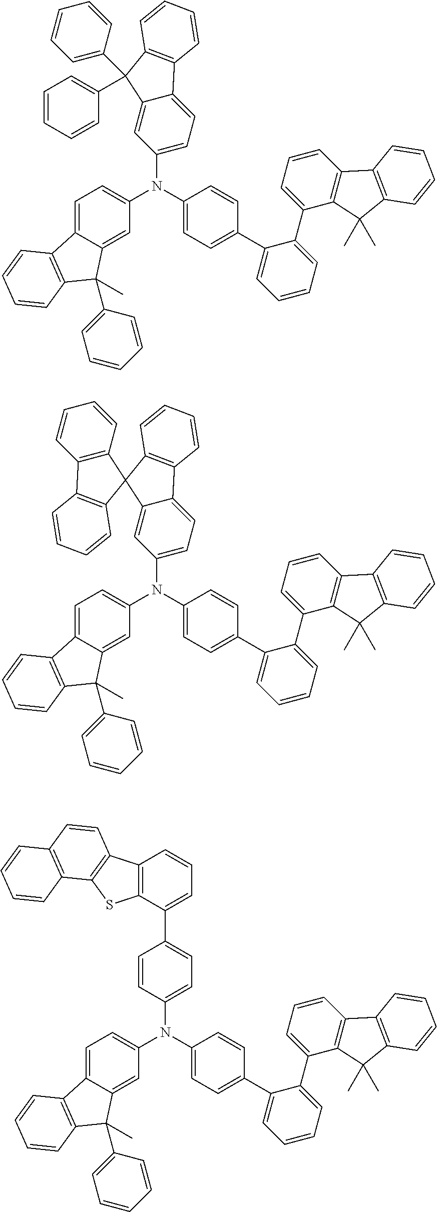

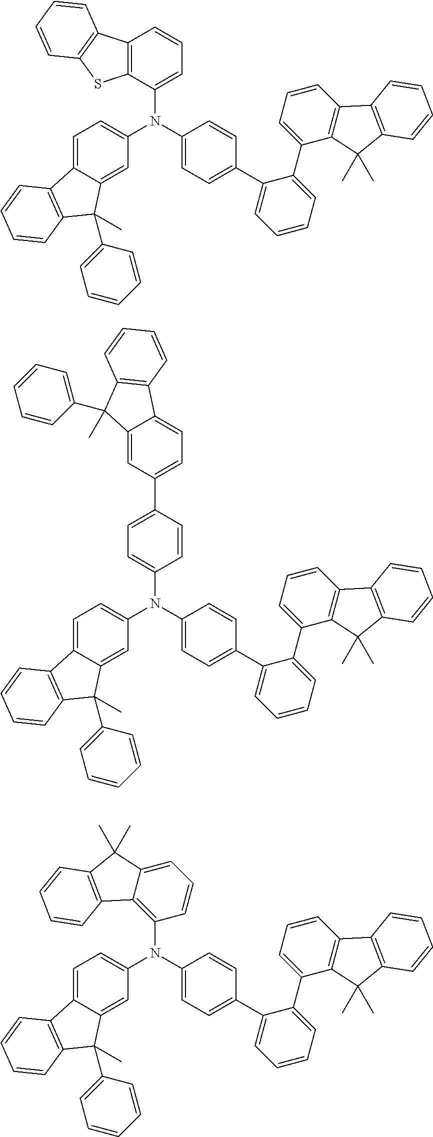

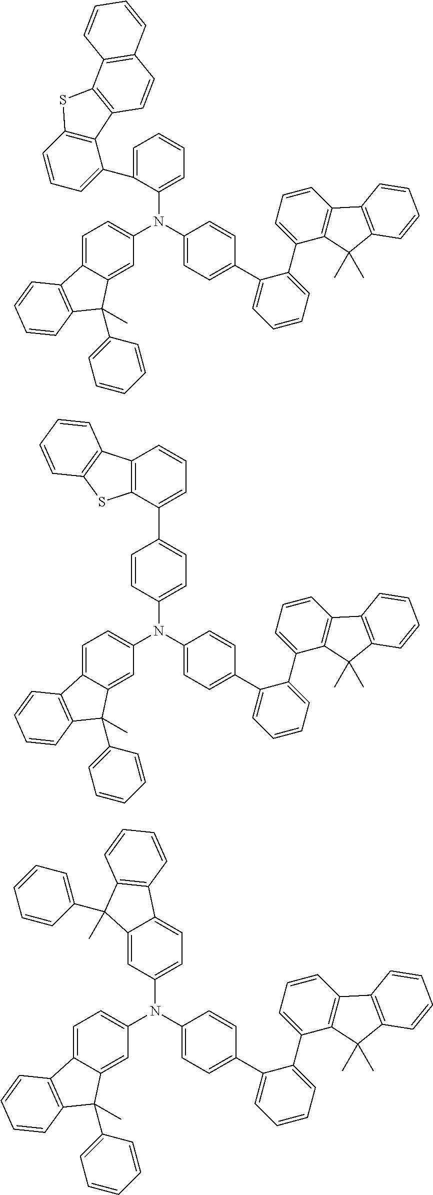

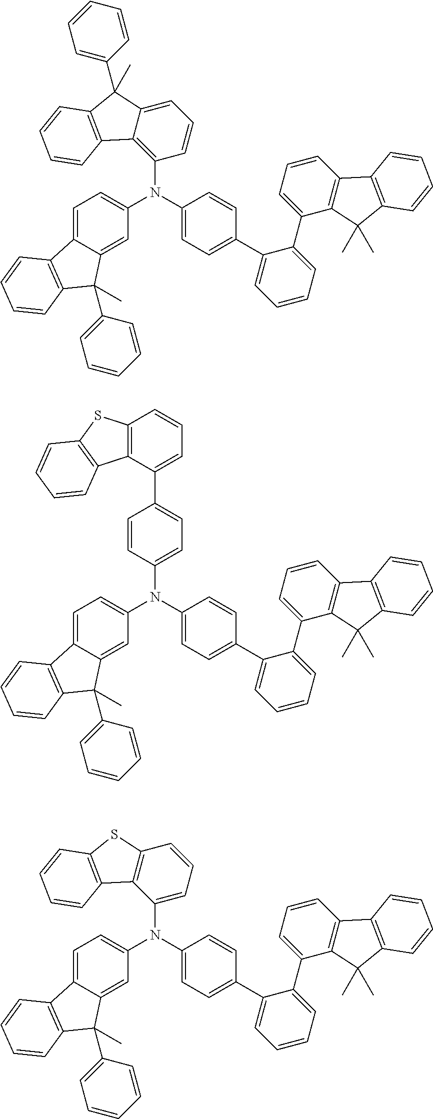 [SMILES: CC1(C)c2ccccc2-c2cccc(-c3ccccc3-c3ccc(N(c4ccc5c(c4)C(C)(c4ccccc4)c4ccccc4-5)c4cccc5c4-c4ccccc4C5(C)c4ccccc4)cc3)c21 ; CC1(C)c2ccccc2-c2cccc(-c3ccccc3-c3ccc(N(c4ccc(-c5cccc6sc7ccccc7c56)cc4)c4ccc5c(c4)C(C)(c4ccccc4)c4ccccc4-5)cc3)c21 ; CC1(C)c2ccccc2-c2cccc(-c3ccccc3-c3ccc(N(c4ccc5c(c4)C(C)(c4ccccc4)c4ccccc4-5)c4cccc5sc6ccccc6c45)cc3)c21]

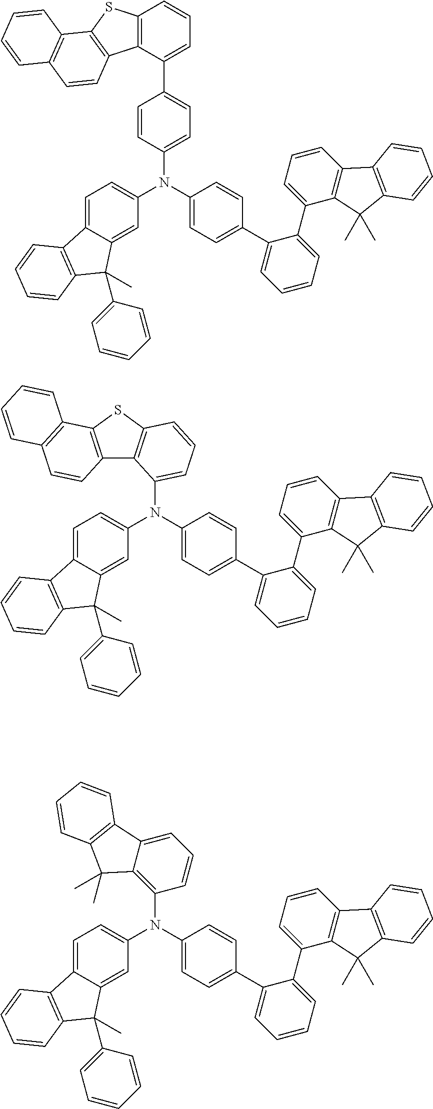 [SMILES: CC1(C)c2ccccc2-c2cccc(-c3ccccc3-c3ccc(N(c4ccc(-c5cccc6sc7c8ccccc8ccc7c56)cc4)c4ccc5c(c4)C(C)(c4ccccc4)c4ccccc4-5)cc3)c21 ; CC1(C)c2ccccc2-c2cccc(-c3ccccc3-c3ccc(N(c4ccc5c(c4)C(C)(c4ccccc4)c4ccccc4-5)c4cccc5sc6c7ccccc7ccc6c45)cc3)c21 ; CC1(C)c2ccccc2-c2cccc(-c3ccccc3-c3ccc(N(c4ccc5c(c4)C(C)(c4ccccc4)c4ccccc4-5)c4cccc5c4C(C)(C)c4ccccc4-5)cc3)c21]

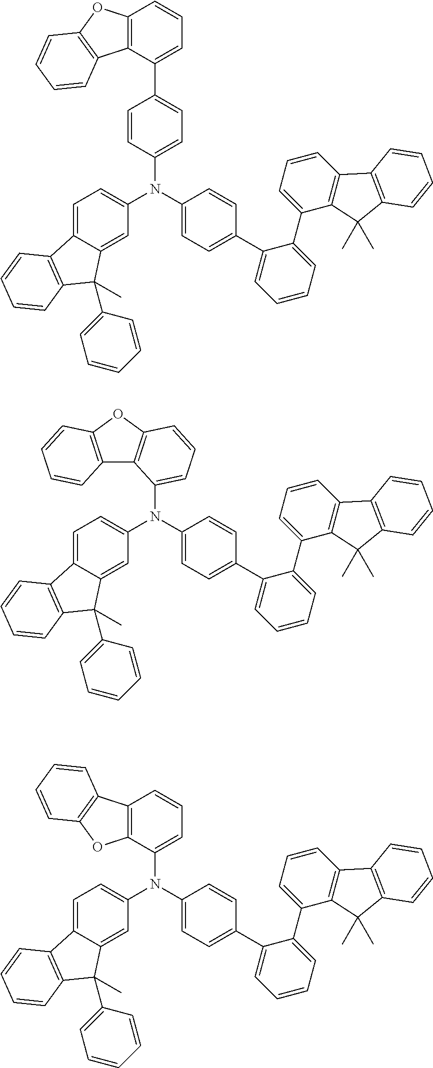

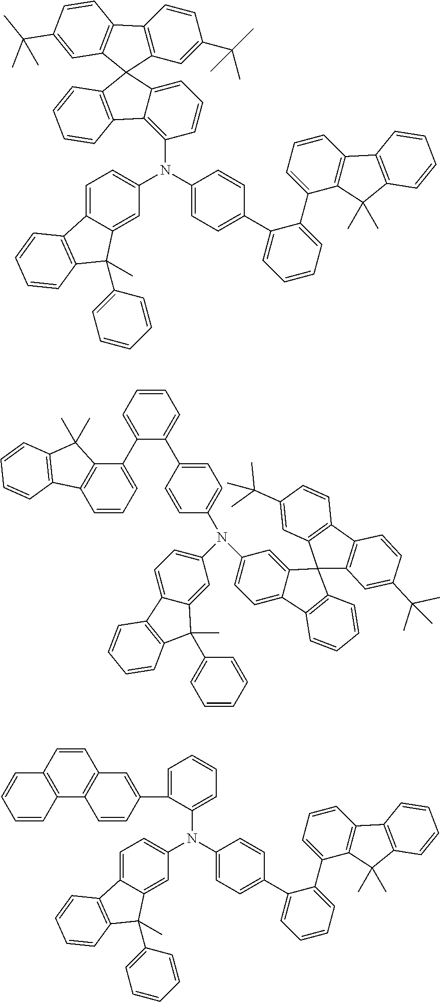 [SMILES: CC(C)(C)c1ccc2c(c1)C1(c3cc(C(C)(C)C)ccc3-2)c2ccccc2-c2c(N(c3ccc(-c4ccccc4-c4cccc5c4C(C)(C)c4ccccc4-5)cc3)c3ccc4c(c3)C(C)(c3ccccc3)c3ccccc3-4)cccc21 ; CC(C)(C)c1ccc2c(c1)C1(c3ccccc3-c3ccc(N(c4ccc(-c5ccccc5-c5cccc6c5C(C)(C)c5ccccc5-6)cc4)c4ccc5c(c4)C(C)(c4ccccc4)c4ccccc4-5)cc31)c1cc(C(C)(C)C)ccc1-2 ; CC1(C)c2ccccc2-c2cccc(-c3ccccc3-c3ccc(N(c4ccc5c(c4)C(C)(c4ccccc4)c4ccccc4-5)c4ccccc4-c4ccc5c(ccc6ccccc65)c4)cc3)c21]

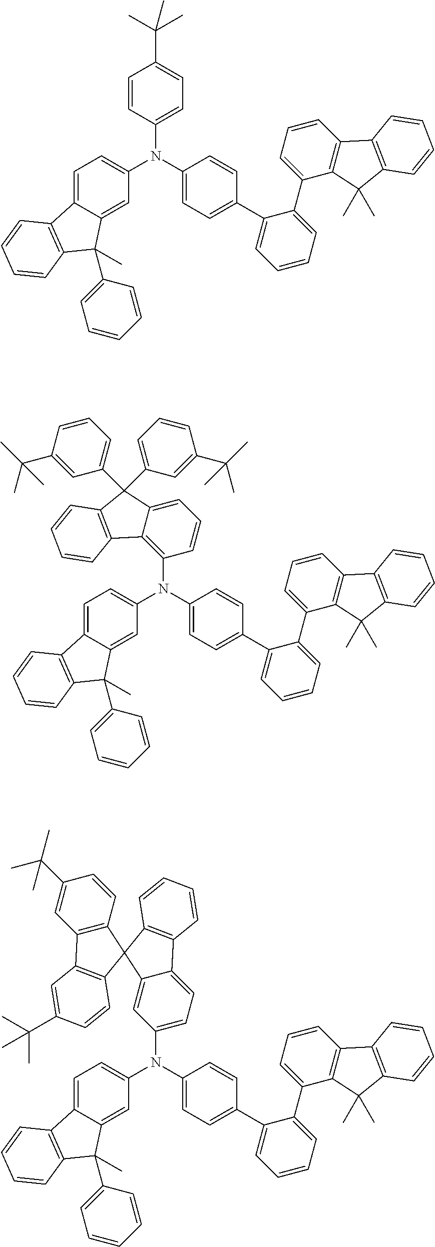 [SMILES: CC(C)(C)c1ccc(N(c2ccc(-c3ccccc3-c3cccc4c3C(C)(C)c3ccccc3-4)cc2)c2ccc3c(c2)C(C)(c2ccccc2)c2ccccc2-3)cc1 ; CC(C)(C)c1cccc(C2(c3cccc(C(C)(C)C)c3)c3ccccc3-c3c(N(c4ccc(-c5ccccc5-c5cccc6c5C(C)(C)c5ccccc5-6)cc4)c4ccc5c(c4)C(C)(c4ccccc4)c4ccccc4-5)cccc32)c1 ; CC(C)(C)c1ccc2c(c1)-c1cc(C(C)(C)C)ccc1C21c2ccccc2-c2ccc(N(c3ccc(-c4ccccc4-c4cccc5c4C(C)(C)c4ccccc4-5)cc3)c3ccc4c(c3)C(C)(c3ccccc3)c3ccccc3-4)cc21]

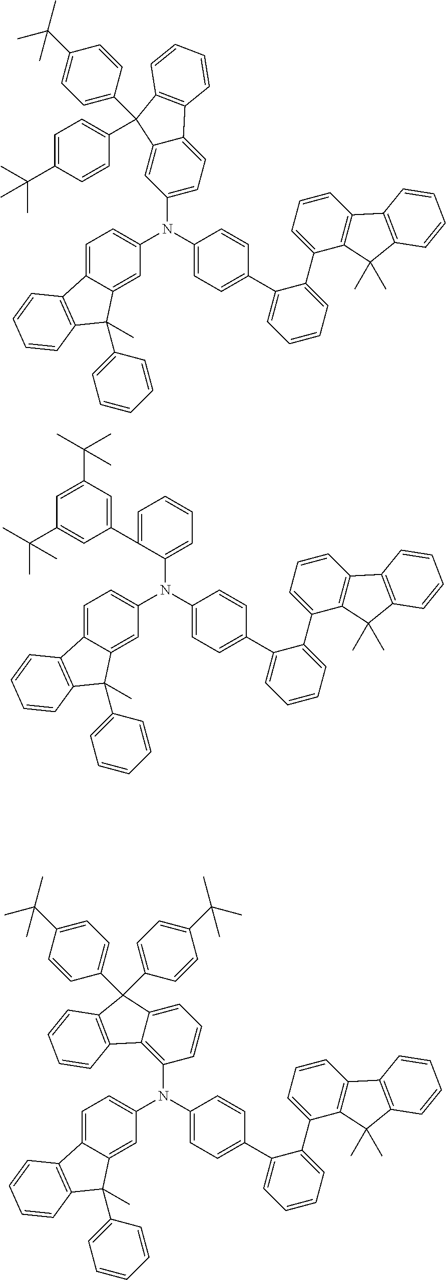

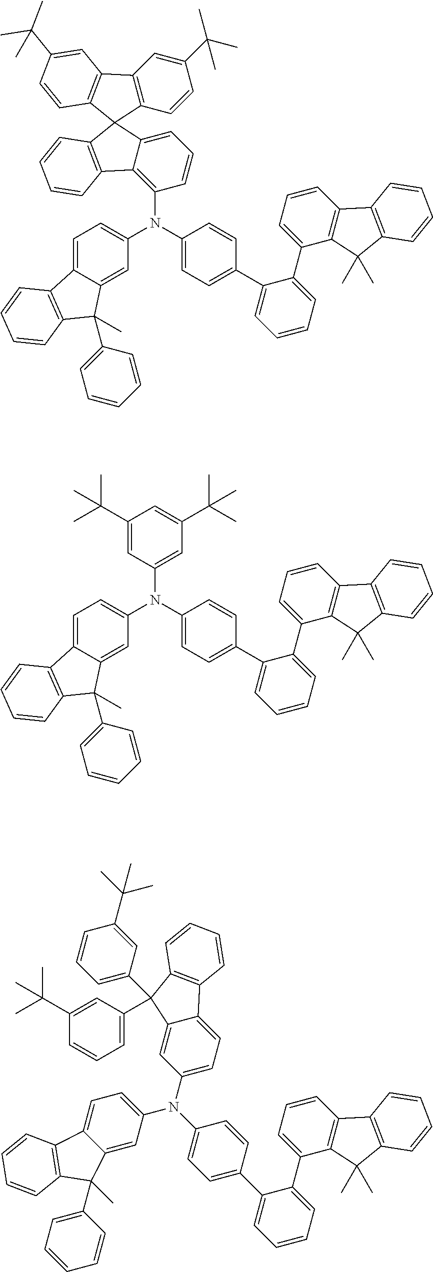 [SMILES: CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C(C)(c3ccccc3)c3ccccc3-4)cccc21 ; CC(C)(C)c1cc(N(c2ccc(-c3ccccc3-c3cccc4c3C(C)(C)c3ccccc3-4)cc2)c2ccc3c(c2)C(C)(c2ccccc2)c2ccccc2-3)cc(C(C)(C)C)c1 ; CC(C)(C)c1cccc(C2(c3cccc(C(C)(C)C)c3)c3ccccc3-c3ccc(N(c4ccc(-c5ccccc5-c5cccc6c5C(C)(C)c5ccccc5-6)cc4)c4ccc5c(c4)C(C)(c4ccccc4)c4ccccc4-5)cc32)c1]

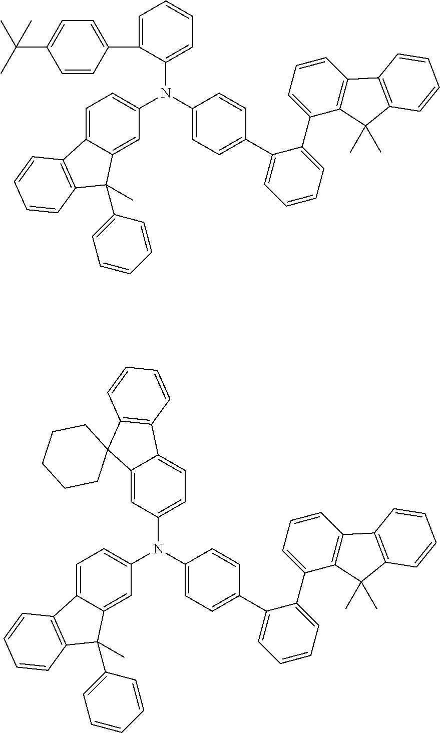 [SMILES: CC(C)(C)c1ccc(-c2ccccc2N(c2ccc(-c3ccccc3-c3cccc4c3C(C)(C)c3ccccc3-4)cc2)c2ccc3c(c2)C(C)(c2ccccc2)c2ccccc2-3)cc1 ; CC1(C)c2ccccc2-c2cccc(-c3ccccc3-c3ccc(N(c4ccc5c(c4)C4(CCCCC4)c4ccccc4-5)c4ccc5c(c4)C(C)(c4ccccc4)c4ccccc4-5)cc3)c21]

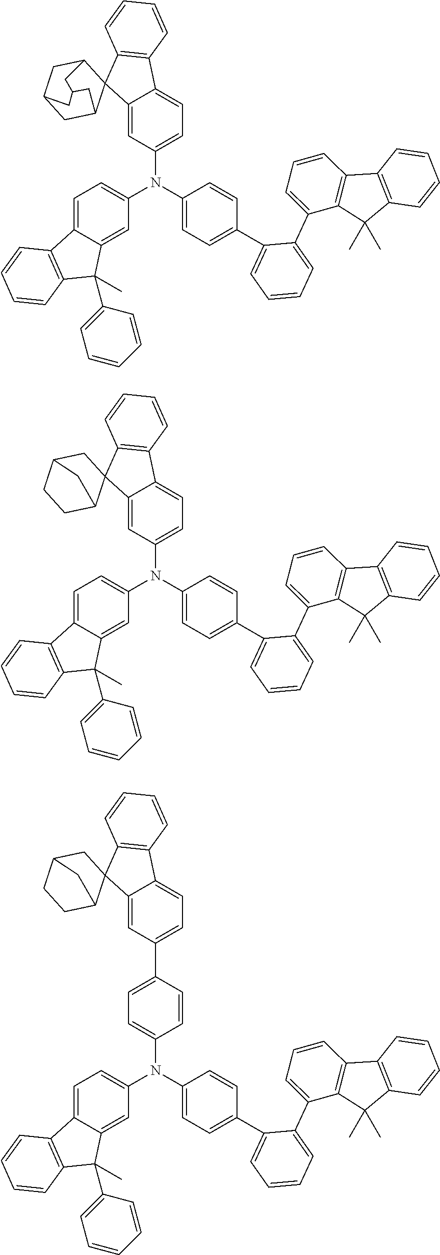

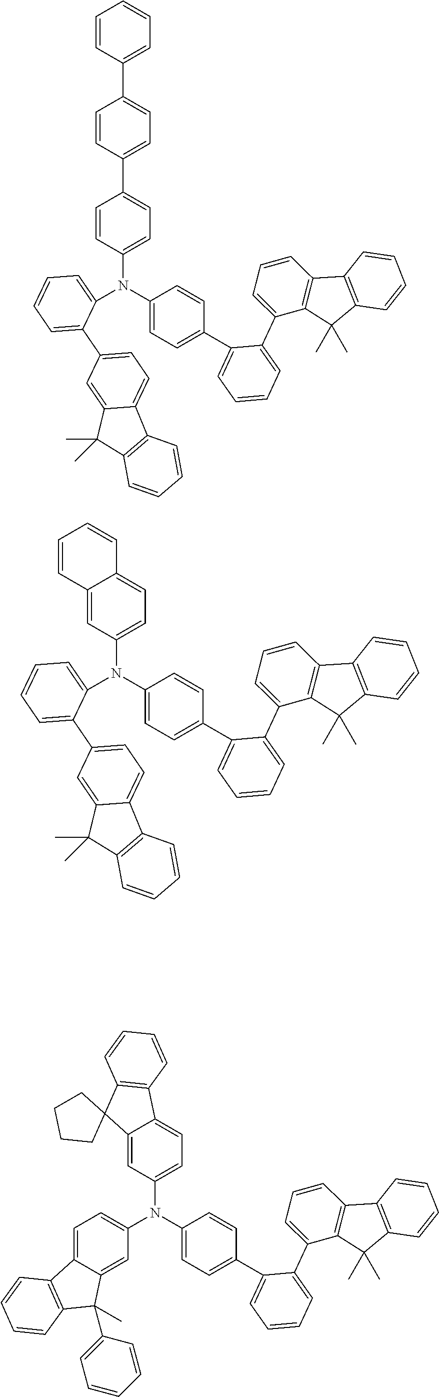 [SMILES: CC1(C)c2ccccc2-c2ccc(-c3ccccc3N(c3ccc(-c4ccc(-c5ccccc5)cc4)cc3)c3ccc(-c4ccccc4-c4cccc5c4C(C)(C)c4ccccc4-5)cc3)cc21 ; CC1(C)c2ccccc2-c2ccc(-c3ccccc3N(c3ccc(-c4ccccc4-c4cccc5c4C(C)(C)c4ccccc4-5)cc3)c3ccc4ccccc4c3)cc21 ; CC1(C)c2ccccc2-c2cccc(-c3ccccc3-c3ccc(N(c4ccc5c(c4)C4(CCCC4)c4ccccc4-5)c4ccc5c(c4)C(C)(c4ccccc4)c4ccccc4-5)cc3)c21]

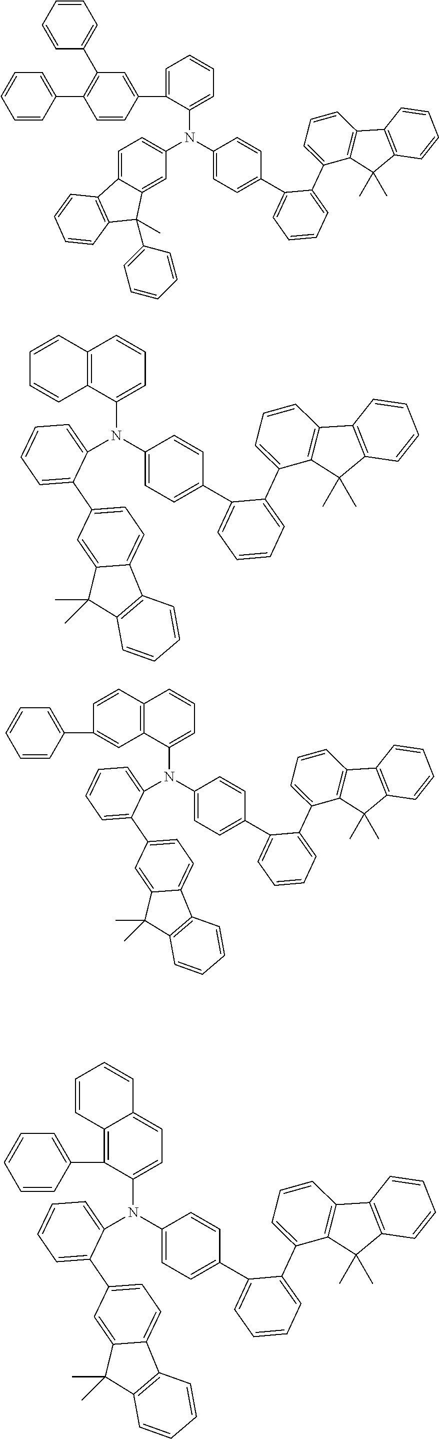

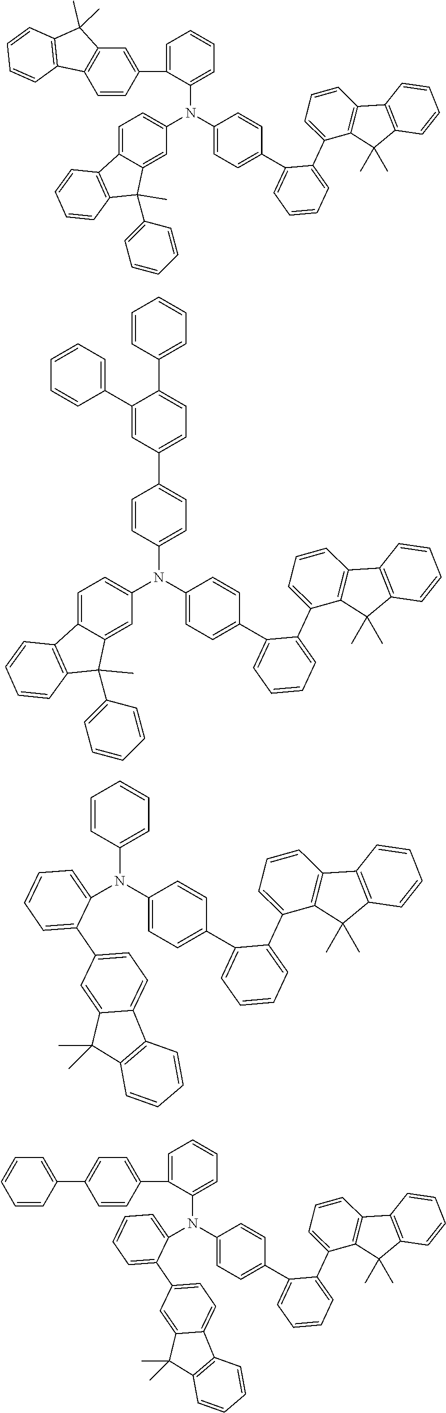

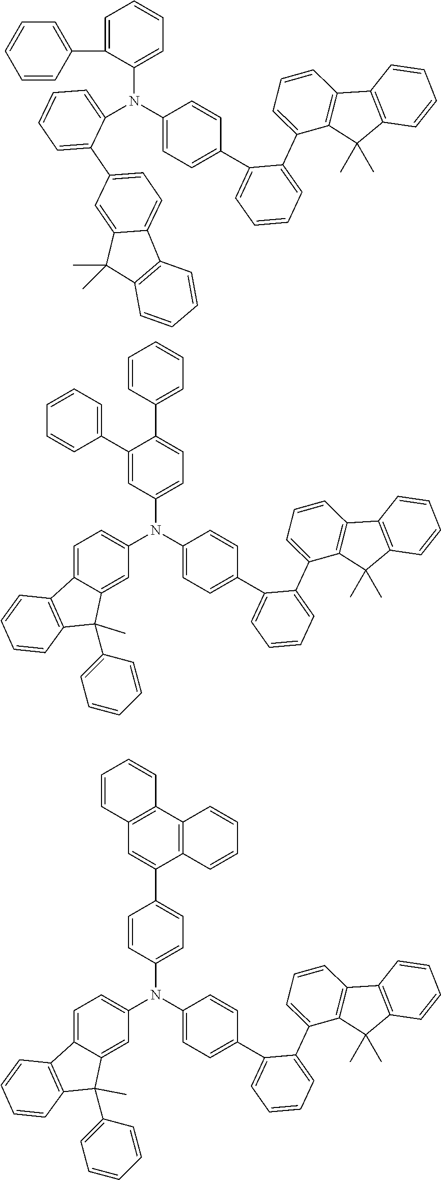

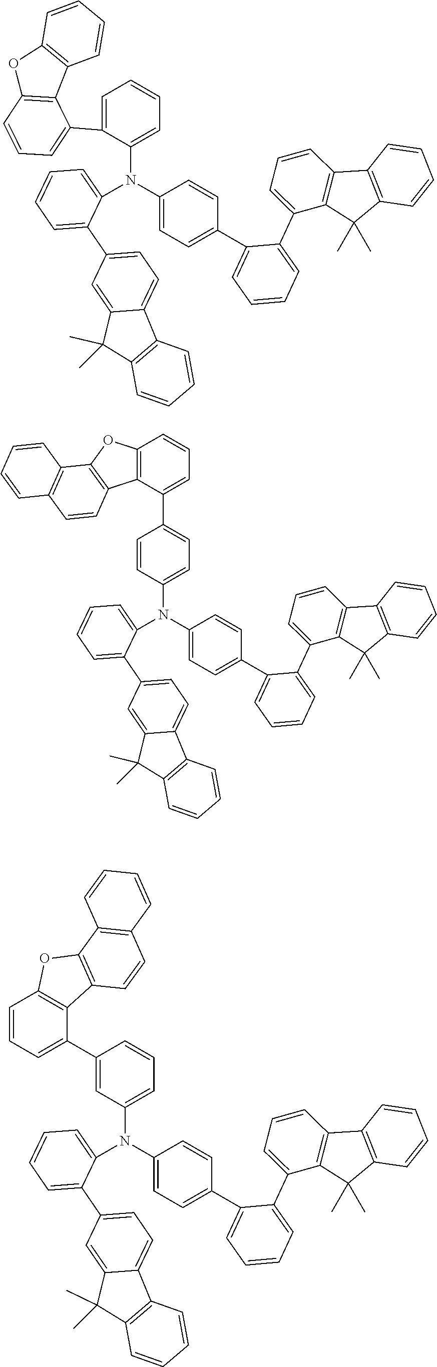

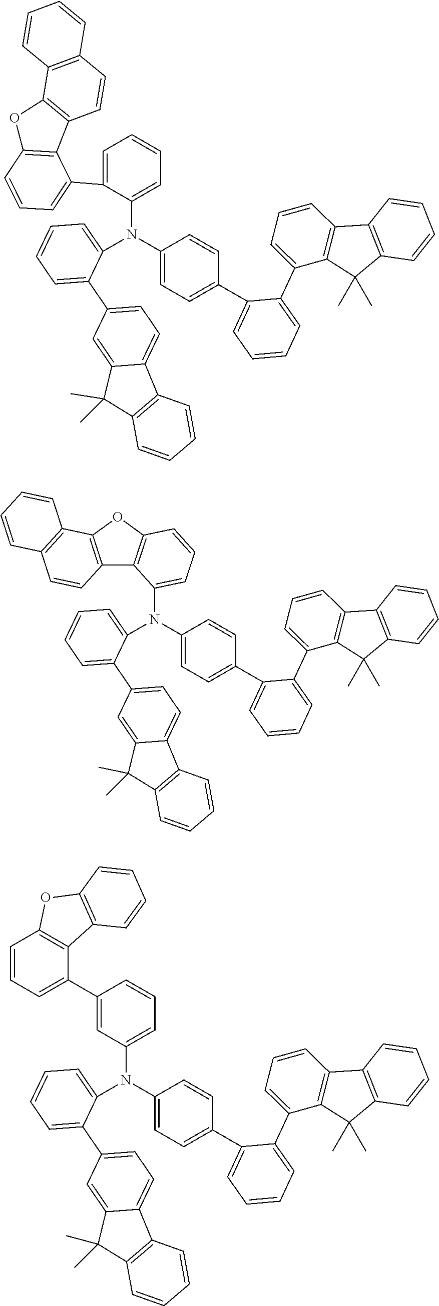 [SMILES: CC1(C)c2ccccc2-c2ccc(-c3ccccc3N(c3ccc(-c4ccccc4-c4cccc5c4C(C)(C)c4ccccc4-5)cc3)c3ccccc3-c3cccc4oc5c6ccccc6ccc5c34)cc21 ; CC1(C)c2ccccc2-c2ccc(-c3ccccc3N(c3ccc(-c4ccccc4-c4cccc5c4C(C)(C)c4ccccc4-5)cc3)c3cccc4oc5c6ccccc6ccc5c34)cc21 ; CC1(C)c2ccccc2-c2ccc(-c3ccccc3N(c3ccc(-c4ccccc4-c4cccc5c4C(C)(C)c4ccccc4-5)cc3)c3cccc(-c4cccc5oc6ccccc6c45)c3)cc21]

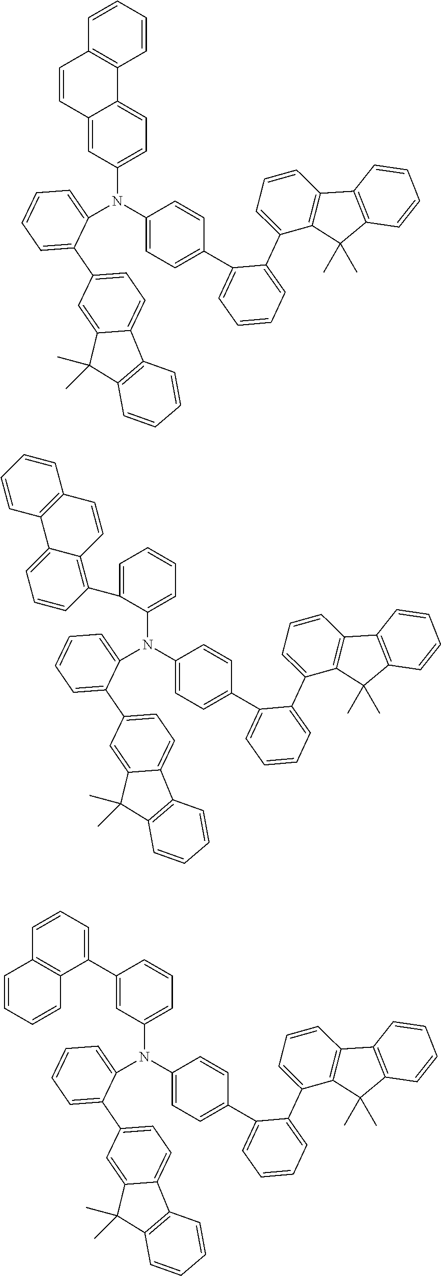 [SMILES: CC1(C)c2ccccc2-c2ccc(-c3ccccc3N(c3ccc(-c4ccccc4-c4cccc5c4C(C)(C)c4ccccc4-5)cc3)c3ccc4c(ccc5ccccc54)c3)cc21 ; CC1(C)c2ccccc2-c2ccc(-c3ccccc3N(c3ccc(-c4ccccc4-c4cccc5c4C(C)(C)c4ccccc4-5)cc3)c3ccccc3-c3cccc4c3ccc3ccccc34)cc21 ; CC1(C)c2ccccc2-c2ccc(-c3ccccc3N(c3ccc(-c4ccccc4-c4cccc5c4C(C)(C)c4ccccc4-5)cc3)c3cccc(-c4cccc5ccccc45)c3)cc21]

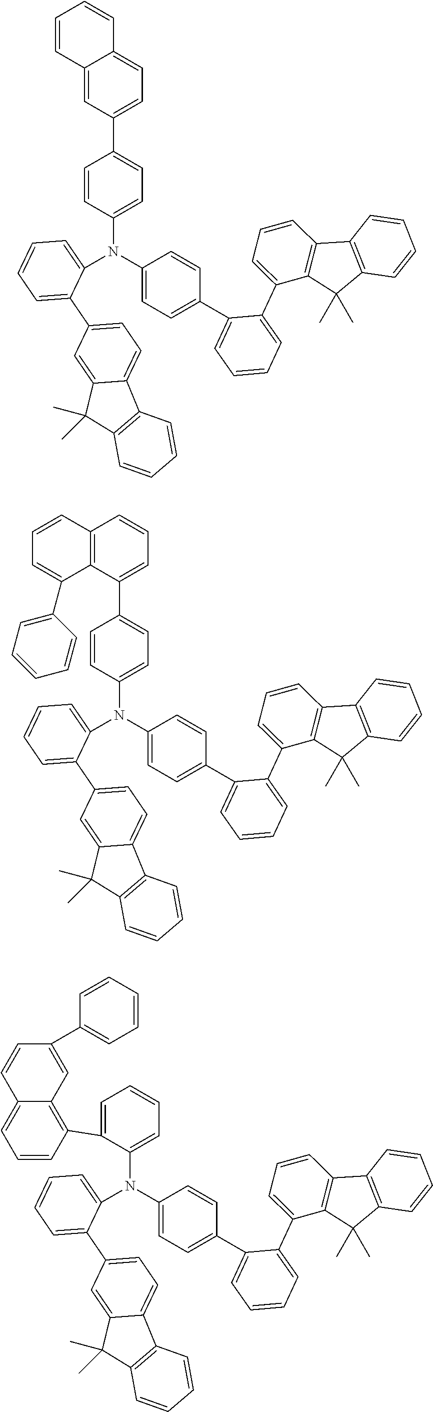 [SMILES: CC1(C)c2ccccc2-c2ccc(-c3ccccc3N(c3ccc(-c4ccc5ccccc5c4)cc3)c3ccc(-c4ccccc4-c4cccc5c4C(C)(C)c4ccccc4-5)cc3)cc21 ; CC1(C)c2ccccc2-c2ccc(-c3ccccc3N(c3ccc(-c4ccccc4-c4cccc5c4C(C)(C)c4ccccc4-5)cc3)c3ccc(-c4cccc5cccc(-c6ccccc6)c45)cc3)cc21 ; CC1(C)c2ccccc2-c2ccc(-c3ccccc3N(c3ccc(-c4ccccc4-c4cccc5c4C(C)(C)c4ccccc4-5)cc3)c3ccccc3-c3cccc4ccc(-c5ccccc5)cc34)cc21]

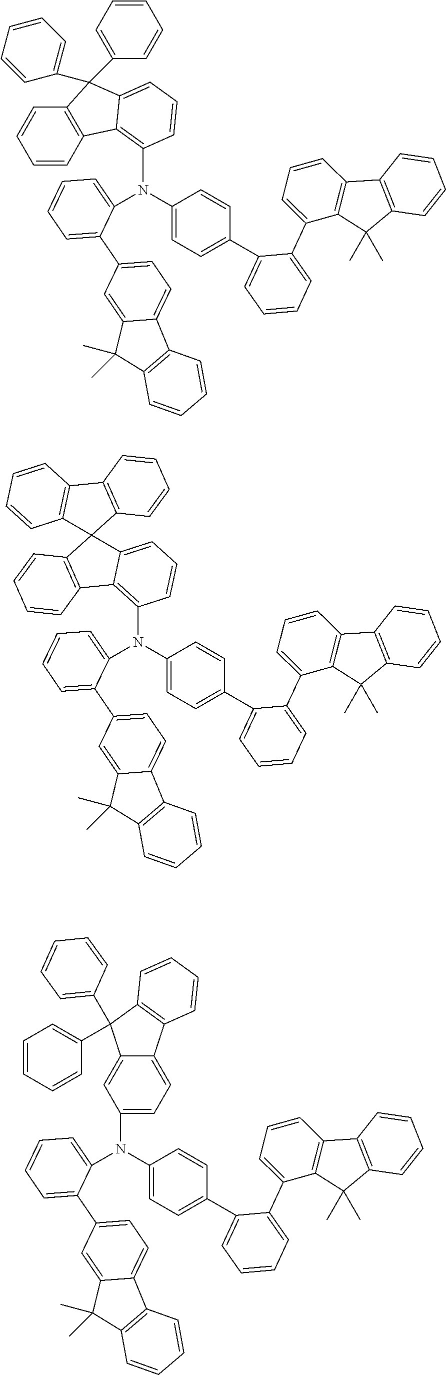 [SMILES: CC1(C)c2ccccc2-c2ccc(-c3ccccc3N(c3ccc(-c4ccccc4-c4cccc5c4C(C)(C)c4ccccc4-5)cc3)c3cccc4c3-c3ccccc3C4(c3ccccc3)c3ccccc3)cc21 ; CC1(C)c2ccccc2-c2ccc(-c3ccccc3N(c3ccc(-c4ccccc4-c4cccc5c4C(C)(C)c4ccccc4-5)cc3)c3cccc4c3-c3ccccc3C43c4ccccc4-c4ccccc43)cc21 ; CC1(C)c2ccccc2-c2ccc(-c3ccccc3N(c3ccc(-c4ccccc4-c4cccc5c4C(C)(C)c4ccccc4-5)cc3)c3ccc4c(c3)C(c3ccccc3)(c3ccccc3)c3ccccc3-4)cc21]

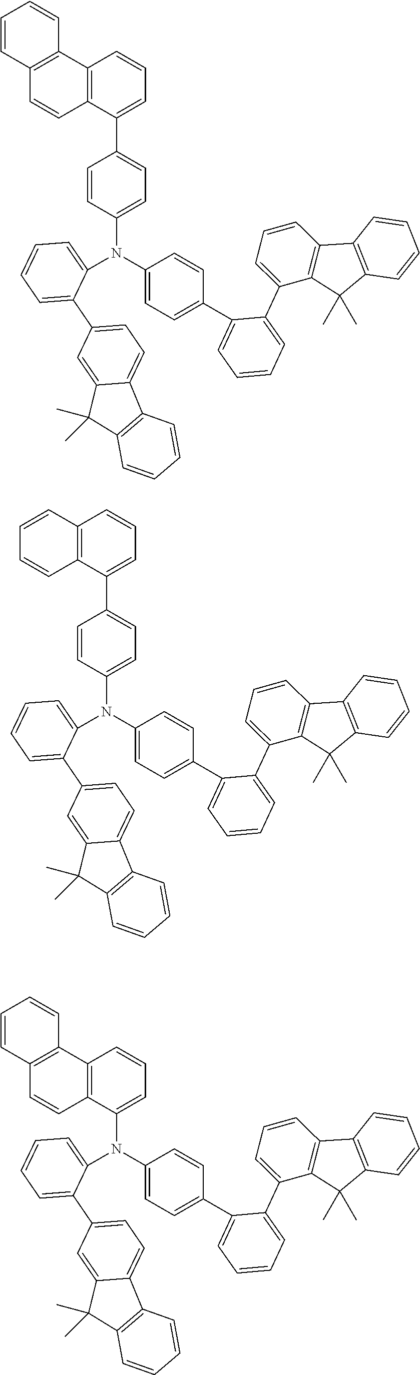

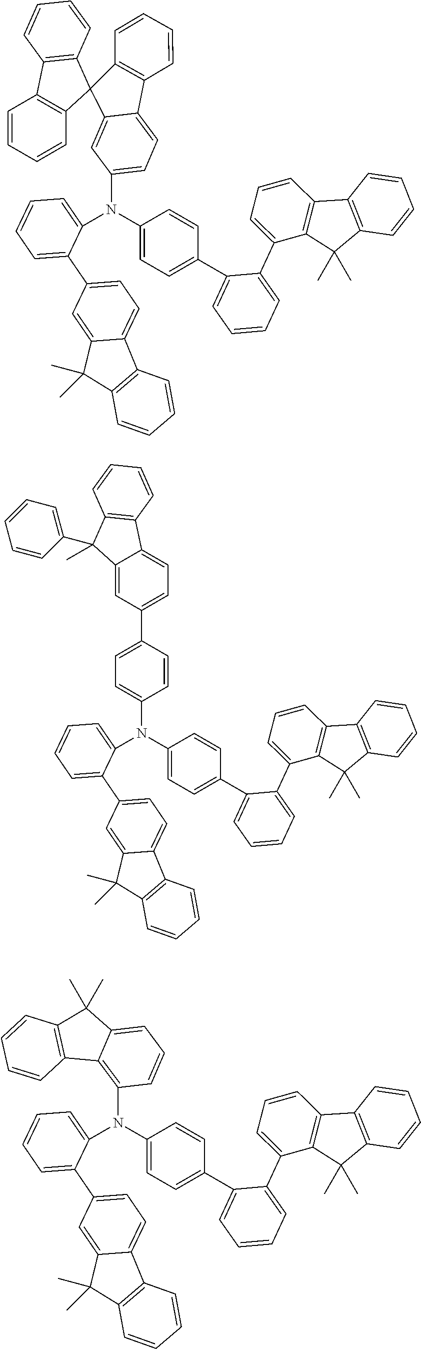

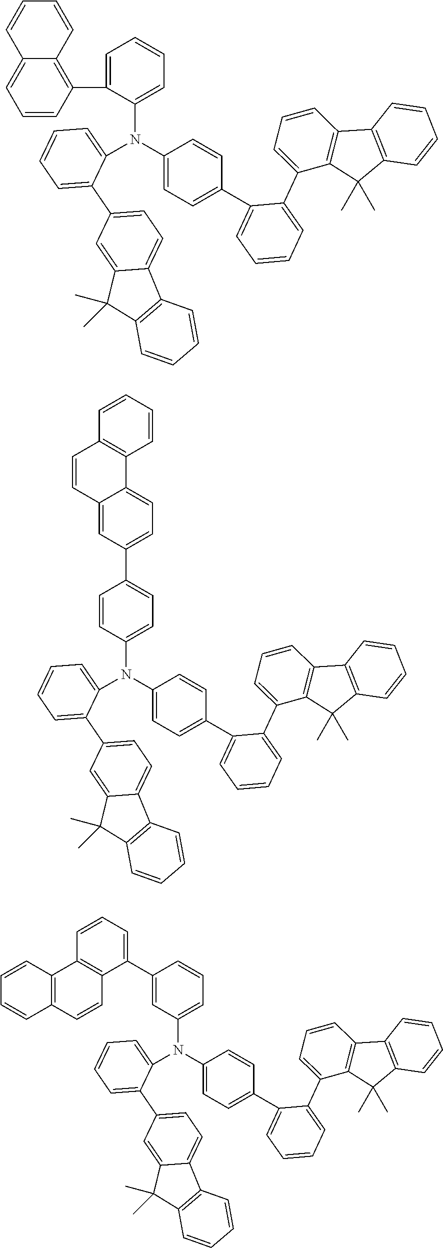 [SMILES: CC1(C)c2ccccc2-c2ccc(-c3ccccc3N(c3ccc(-c4ccccc4-c4cccc5c4C(C)(C)c4ccccc4-5)cc3)c3ccccc3-c3cccc4ccccc34)cc21 ; CC1(C)c2ccccc2-c2ccc(-c3ccccc3N(c3ccc(-c4ccc5c(ccc6ccccc65)c4)cc3)c3ccc(-c4ccccc4-c4cccc5c4C(C)(C)c4ccccc4-5)cc3)cc21 ; CC1(C)c2ccccc2-c2ccc(-c3ccccc3N(c3ccc(-c4ccccc4-c4cccc5c4C(C)(C)c4ccccc4-5)cc3)c3cccc(-c4cccc5c4ccc4ccccc45)c3)cc21]

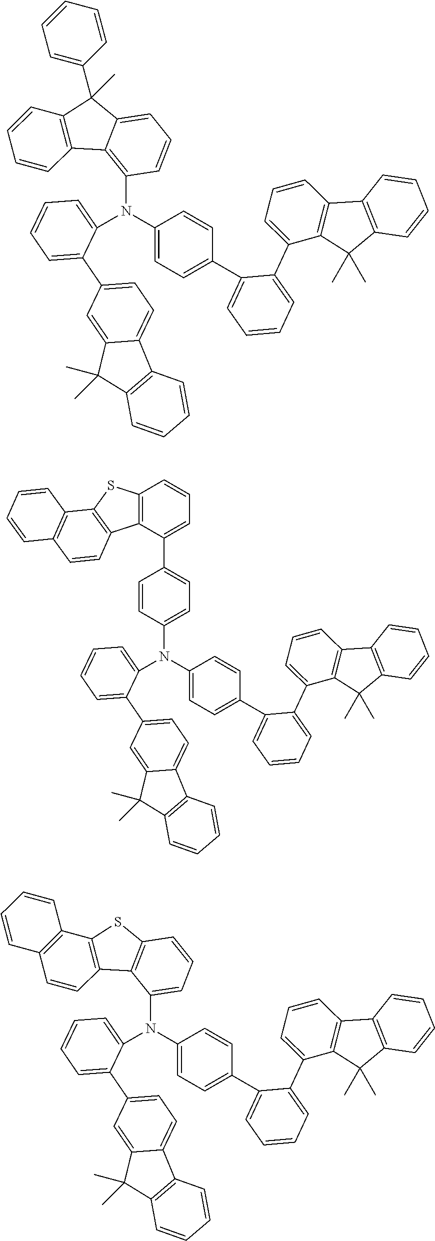

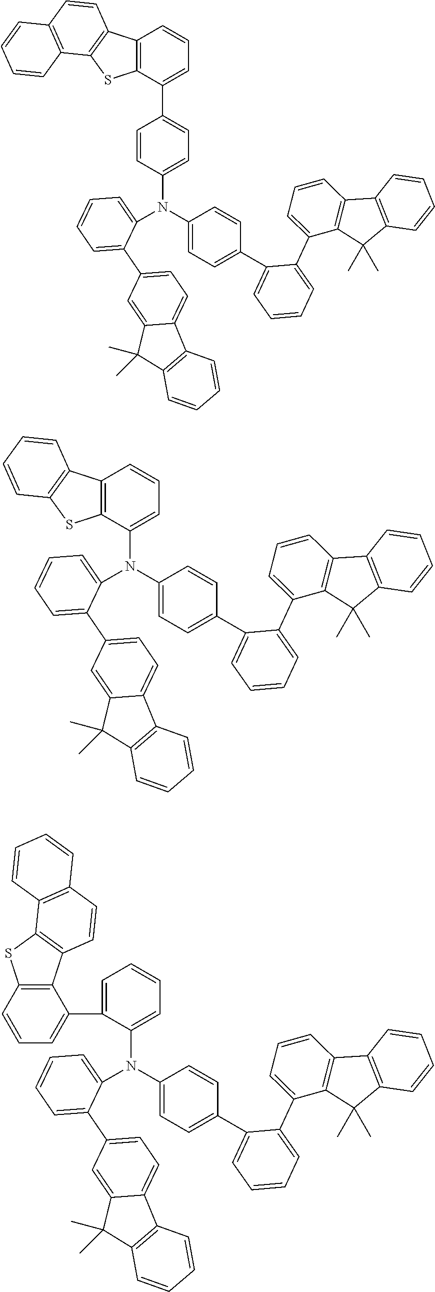 [SMILES: CC1(C)c2ccccc2-c2ccc(-c3ccccc3N(c3ccc(-c4ccccc4-c4cccc5c4C(C)(C)c4ccccc4-5)cc3)c3ccc(-c4cccc5c4sc4c6ccccc6ccc54)cc3)cc21 ; CC1(C)c2ccccc2-c2ccc(-c3ccccc3N(c3ccc(-c4ccccc4-c4cccc5c4C(C)(C)c4ccccc4-5)cc3)c3cccc4c3sc3ccccc34)cc21 ; CC1(C)c2ccccc2-c2ccc(-c3ccccc3N(c3ccc(-c4ccccc4-c4cccc5c4C(C)(C)c4ccccc4-5)cc3)c3ccccc3-c3cccc4sc5c6ccccc6ccc5c34)cc21]

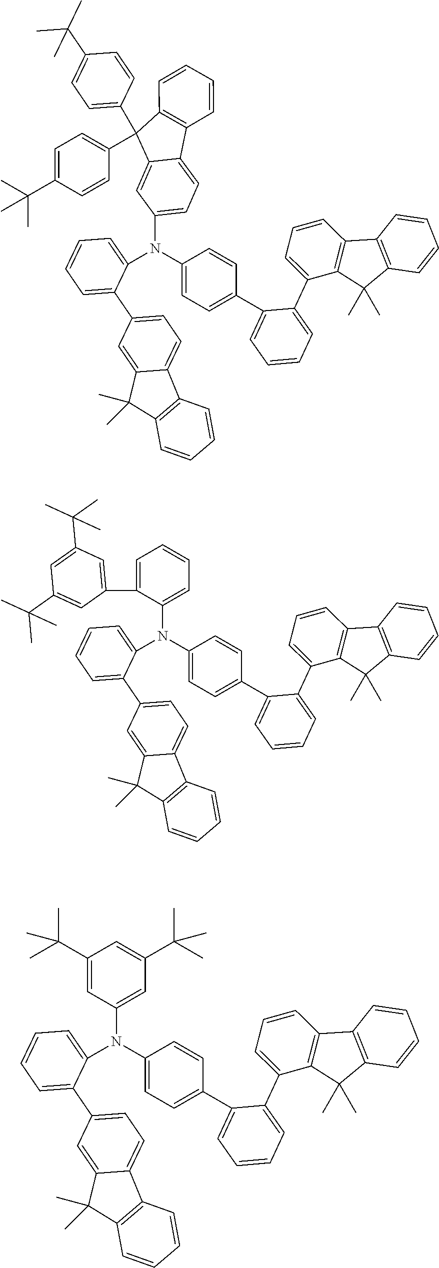 [SMILES: CC(C)(C)c1ccc(C2(c3ccc(C(C)(C)C)cc3)c3ccccc3-c3ccc(N(c4ccc(-c5ccccc5-c5cccc6c5C(C)(C)c5ccccc5-6)cc4)c4ccccc4-c4ccc5c(c4)C(C)(C)c4ccccc4-5)cc32)cc1 ; CC(C)(C)c1cc(-c2ccccc2N(c2ccc(-c3ccccc3-c3cccc4c3C(C)(C)c3ccccc3-4)cc2)c2ccccc2-c2ccc3c(c2)C(C)(C)c2ccccc2-3)cc(C(C)(C)C)c1 ; CC(C)(C)c1cc(N(c2ccc(-c3ccccc3-c3cccc4c3C(C)(C)c3ccccc3-4)cc2)c2ccccc2-c2ccc3c(c2)C(C)(C)c2ccccc2-3)cc(C(C)(C)C)c1]

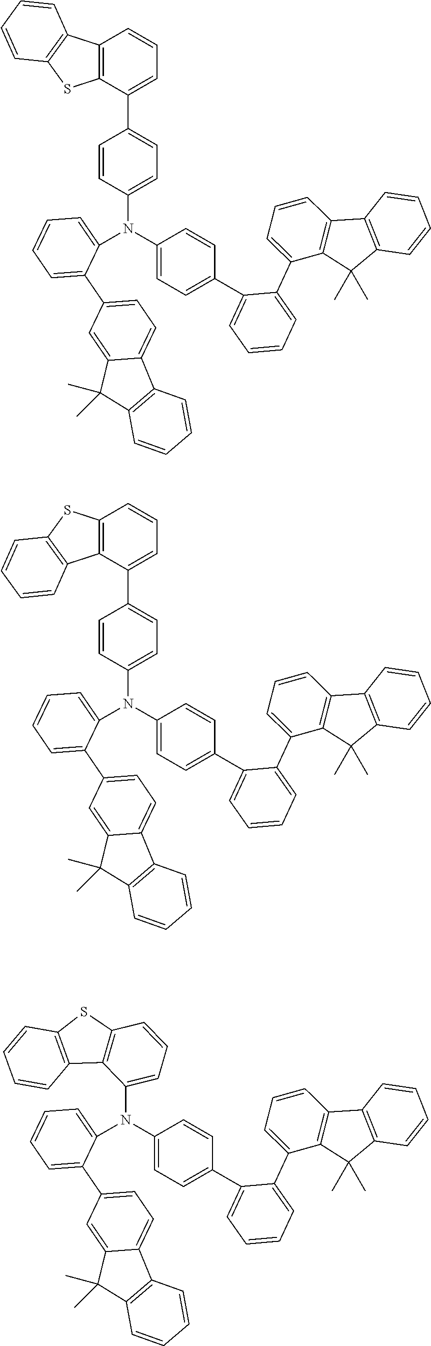 [SMILES: CC1(C)c2ccccc2-c2ccc(-c3ccccc3N(c3ccc(-c4ccccc4-c4cccc5c4C(C)(C)c4ccccc4-5)cc3)c3ccc(-c4cccc5c4sc4ccccc45)cc3)cc21 ; CC1(C)c2ccccc2-c2ccc(-c3ccccc3N(c3ccc(-c4ccccc4-c4cccc5c4C(C)(C)c4ccccc4-5)cc3)c3ccc(-c4cccc5sc6ccccc6c45)cc3)cc21 ; CC1(C)c2ccccc2-c2ccc(-c3ccccc3N(c3ccc(-c4ccccc4-c4cccc5c4C(C)(C)c4ccccc4-5)cc3)c3cccc4sc5ccccc5c34)cc21]

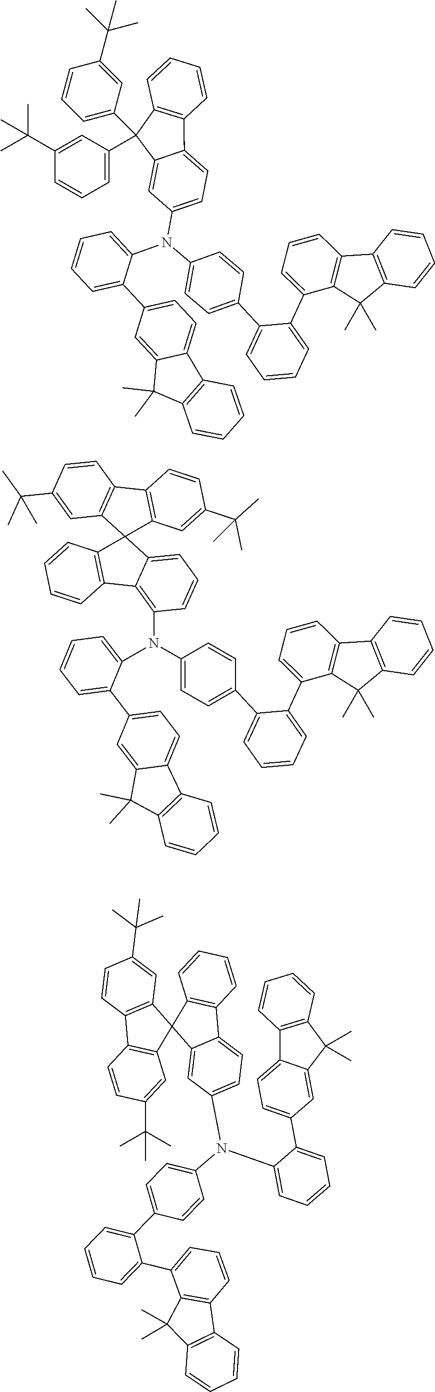

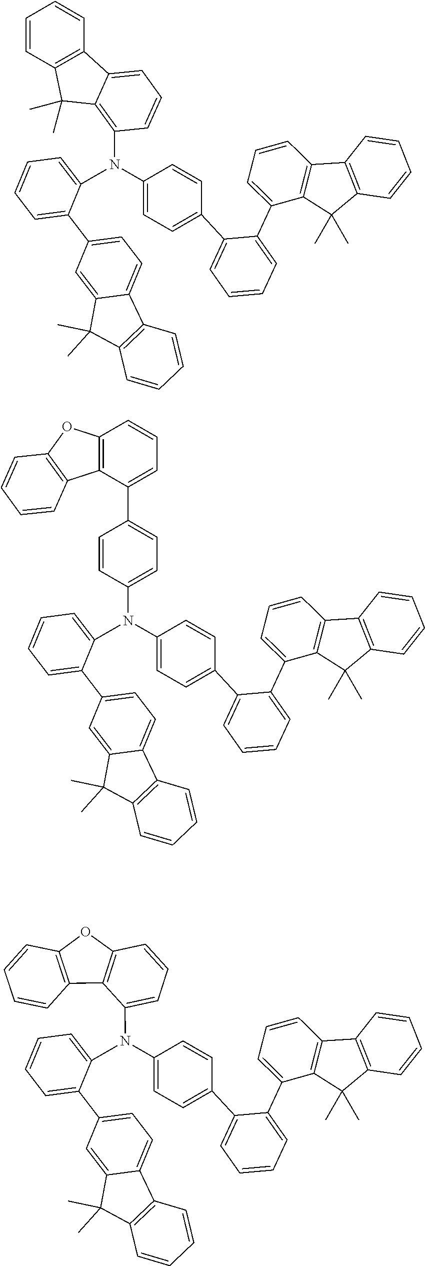

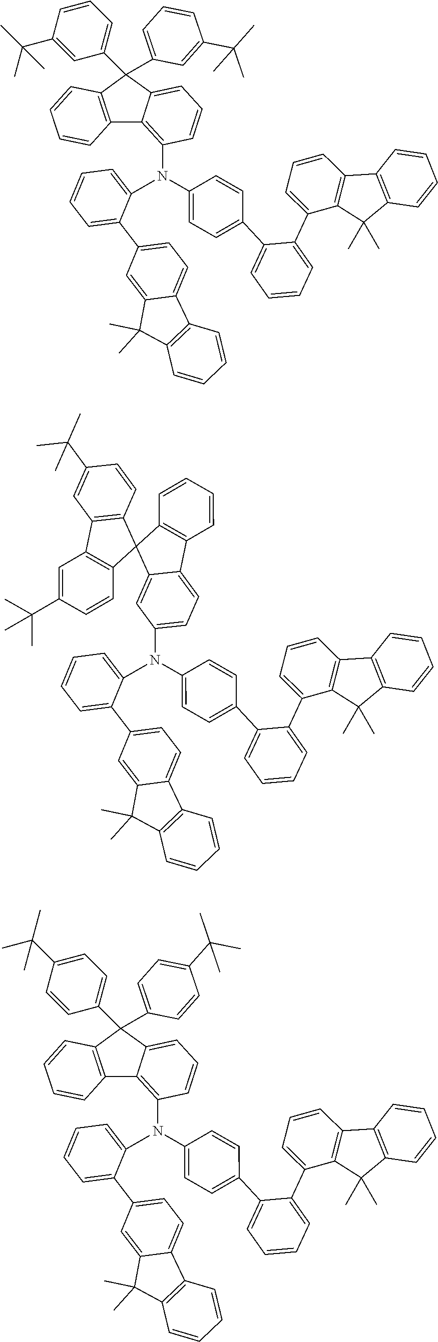

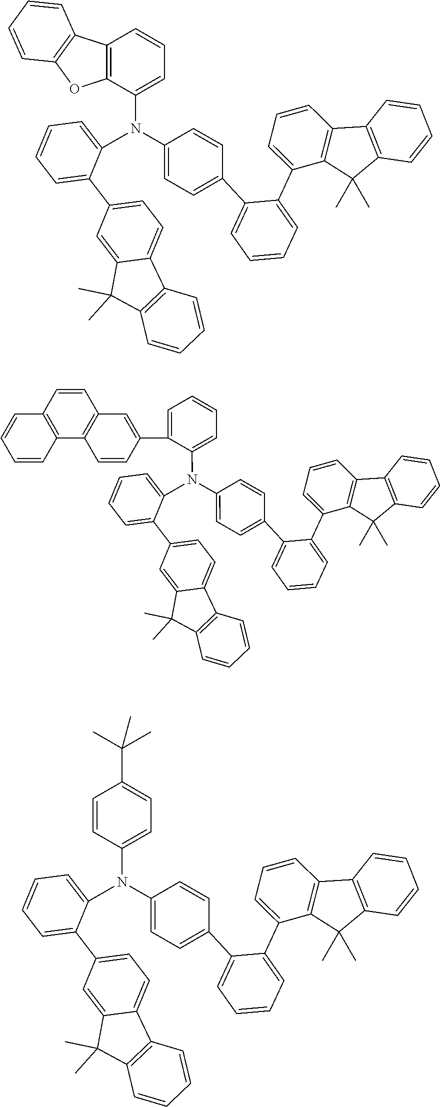

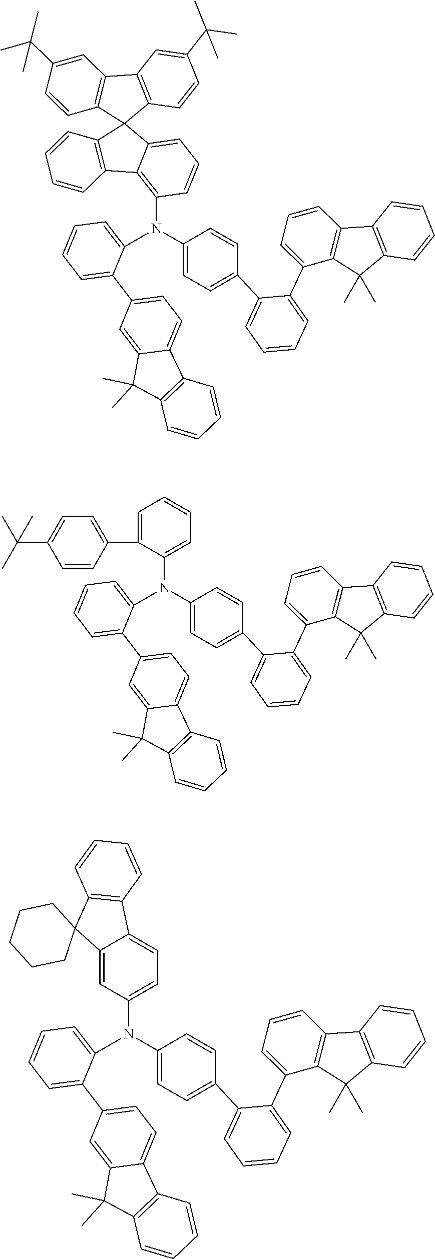 [SMILES: CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccccc3-c3ccc4c(c3)C(C)(C)c3ccccc3-4)cccc21 ; CC(C)(C)c1ccc(-c2ccccc2N(c2ccc(-c3ccccc3-c3cccc4c3C(C)(C)c3ccccc3-4)cc2)c2ccccc2-c2ccc3c(c2)C(C)(C)c2ccccc2-3)cc1 ; CC1(C)c2ccccc2-c2ccc(-c3ccccc3N(c3ccc(-c4ccccc4-c4cccc5c4C(C)(C)c4ccccc4-5)cc3)c3ccc4c(c3)C3(CCCCC3)c3ccccc3-4)cc21]

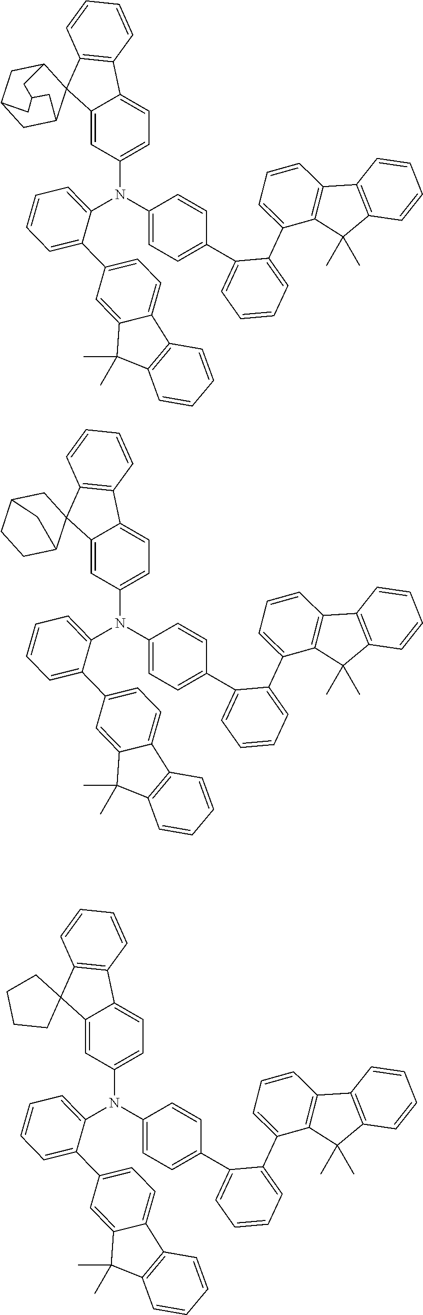

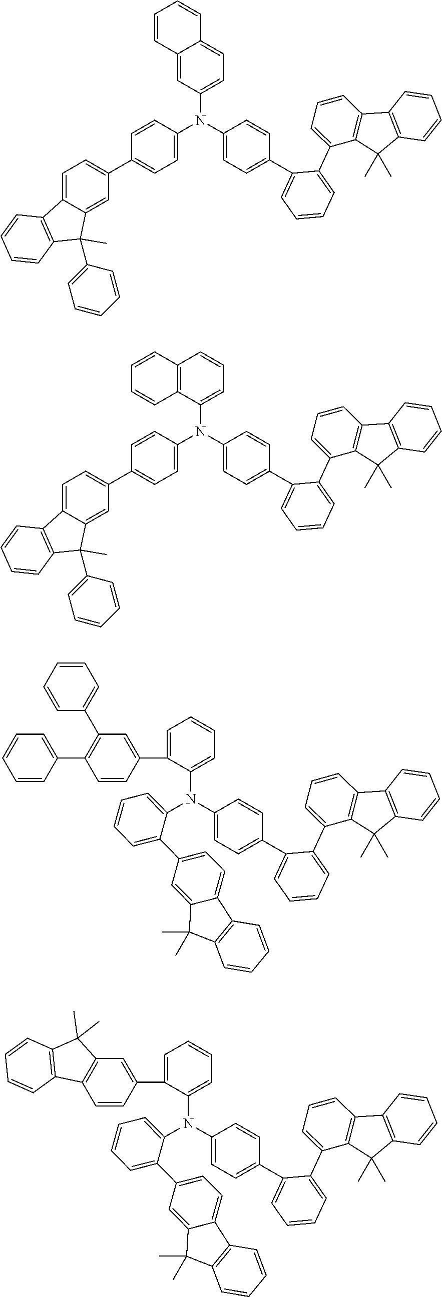 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4ccc5ccccc5c4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4cccc5ccccc45)cc3)c21 ; CC1(C)c2ccccc2-c2ccc(-c3ccccc3N(c3ccc(-c4ccccc4-c4cccc5c4C(C)(C)c4ccccc4-5)cc3)c3ccccc3-c3ccc(-c4ccccc4)c(-c4ccccc4)c3)cc21 ; CC1(C)c2ccccc2-c2ccc(-c3ccccc3N(c3ccc(-c4ccccc4-c4cccc5c4C(C)(C)c4ccccc4-5)cc3)c3ccccc3-c3ccc4c(c3)C(C)(C)c3ccccc3-4)cc21]

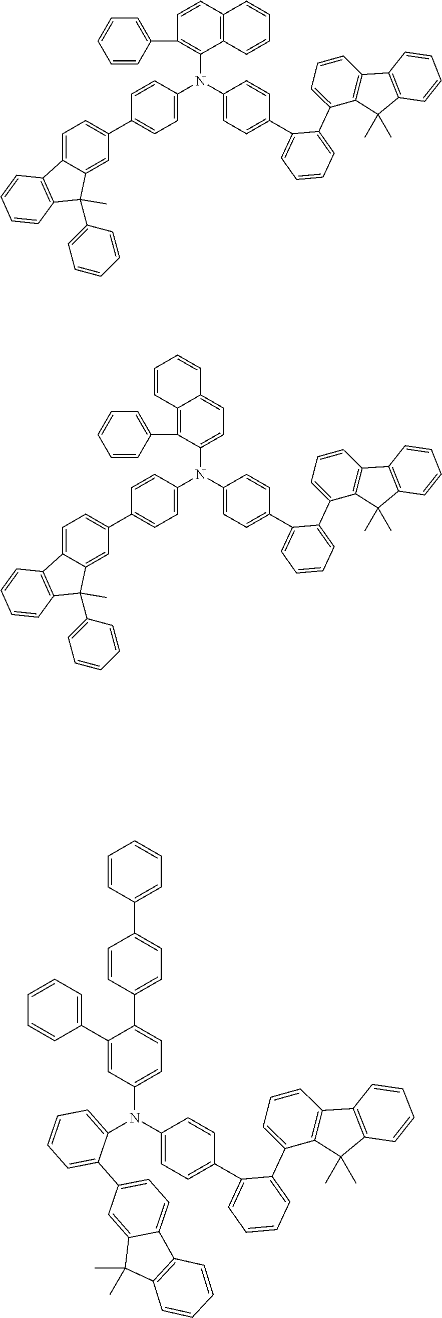 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4c(-c5ccccc5)ccc5ccccc45)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc5ccccc5c4-c4ccccc4)cc3)c21 ; CC1(C)c2ccccc2-c2ccc(-c3ccccc3N(c3ccc(-c4ccccc4-c4cccc5c4C(C)(C)c4ccccc4-5)cc3)c3ccc(-c4ccc(-c5ccccc5)cc4)c(-c4ccccc4)c3)cc21]

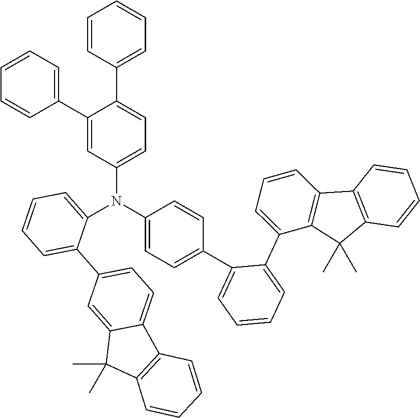 [SMILES: CC1(C)c2ccccc2-c2ccc(-c3ccccc3N(c3ccc(-c4ccccc4-c4cccc5c4C(C)(C)c4ccccc4-5)cc3)c3ccc(-c4ccccc4)c(-c4ccccc4)c3)cc21]

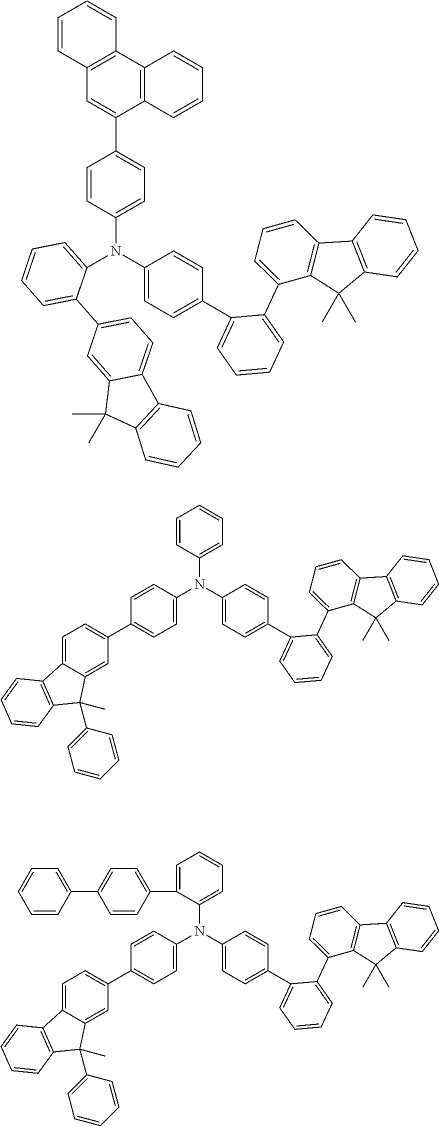 [SMILES: CC1(C)c2ccccc2-c2ccc(-c3ccccc3N(c3ccc(-c4ccccc4-c4cccc5c4C(C)(C)c4ccccc4-5)cc3)c3ccc(-c4cc5ccccc5c5ccccc45)cc3)cc21 ; CC1(C)c2ccccc2-c2cccc(-c3ccccc3-c3ccc(N(c4ccccc4)c4ccc(-c5ccc6c(c5)C(C)(c5ccccc5)c5ccccc5-6)cc4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccccc4-c4ccc(-c5ccccc5)cc4)cc3)c21]

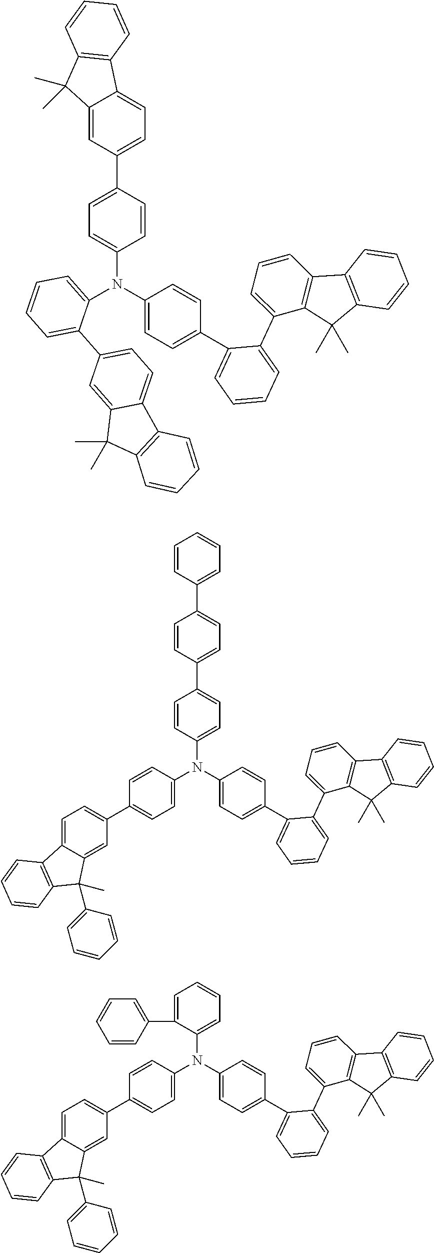 [SMILES: CC1(C)c2ccccc2-c2ccc(-c3ccc(N(c4ccc(-c5ccccc5-c5cccc6c5C(C)(C)c5ccccc5-6)cc4)c4ccccc4-c4ccc5c(c4)C(C)(C)c4ccccc4-5)cc3)cc21 ; CC1(C)c2ccccc2-c2cccc(-c3ccccc3-c3ccc(N(c4ccc(-c5ccc(-c6ccccc6)cc5)cc4)c4ccc(-c5ccc6c(c5)C(C)(c5ccccc5)c5ccccc5-6)cc4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccccc4-c4ccccc4)cc3)c21]

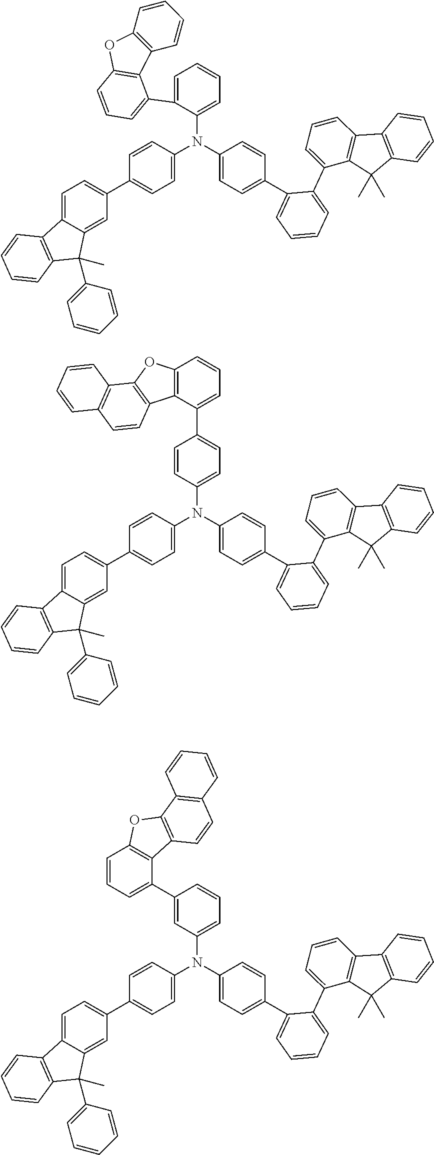

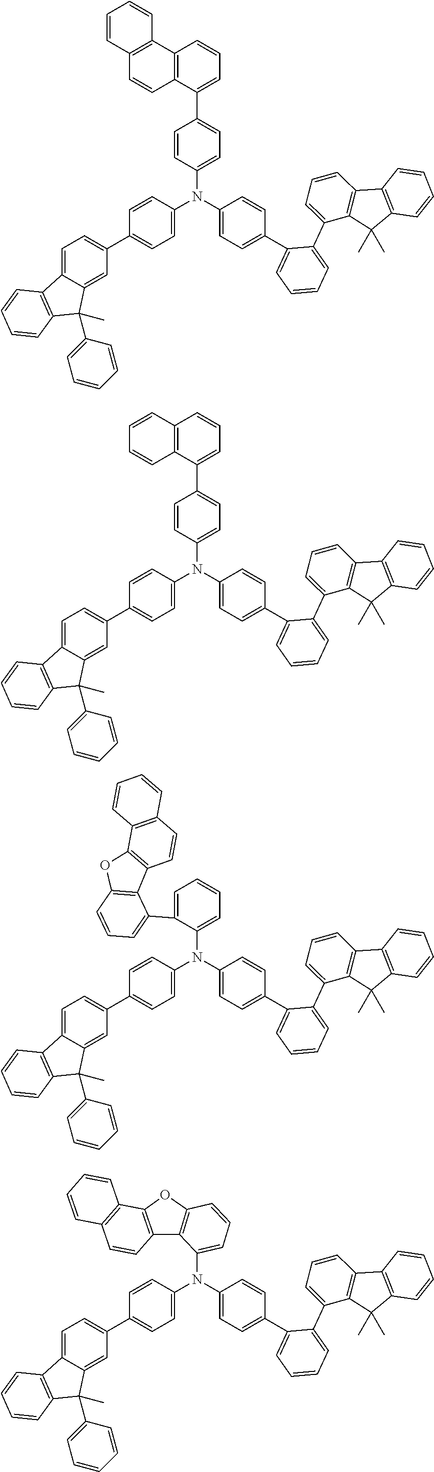

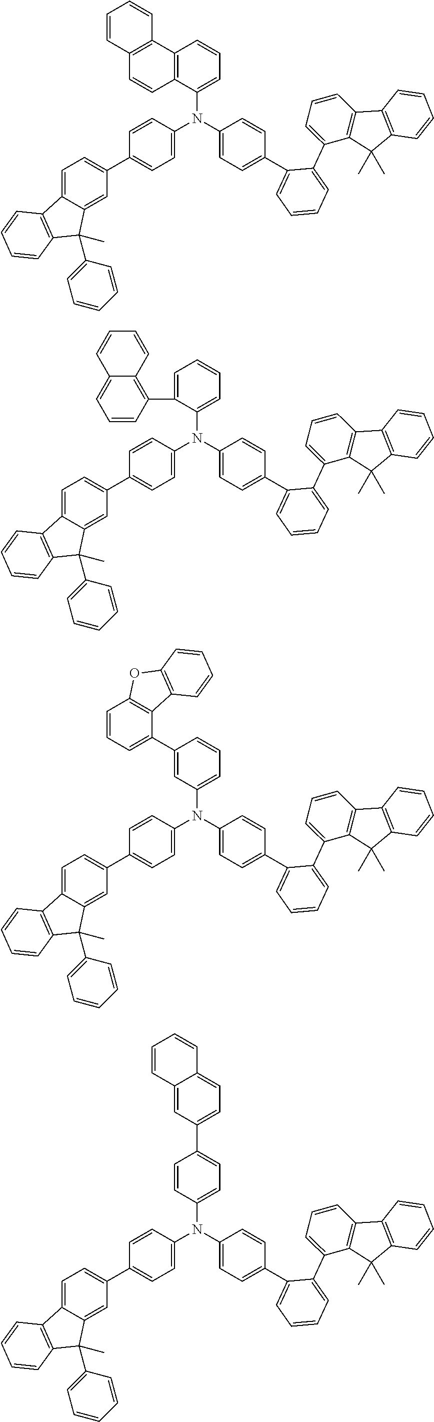

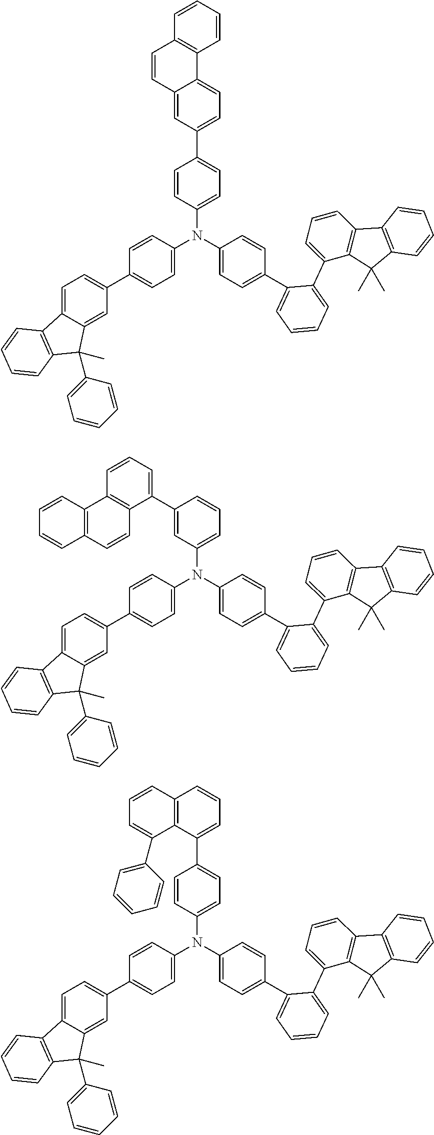 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4ccc(-c5ccc6c(ccc7ccccc76)c5)cc4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4cccc(-c5cccc6c5ccc5ccccc56)c4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc(-c5cccc6cccc(-c7ccccc7)c56)cc4)cc3)c21]

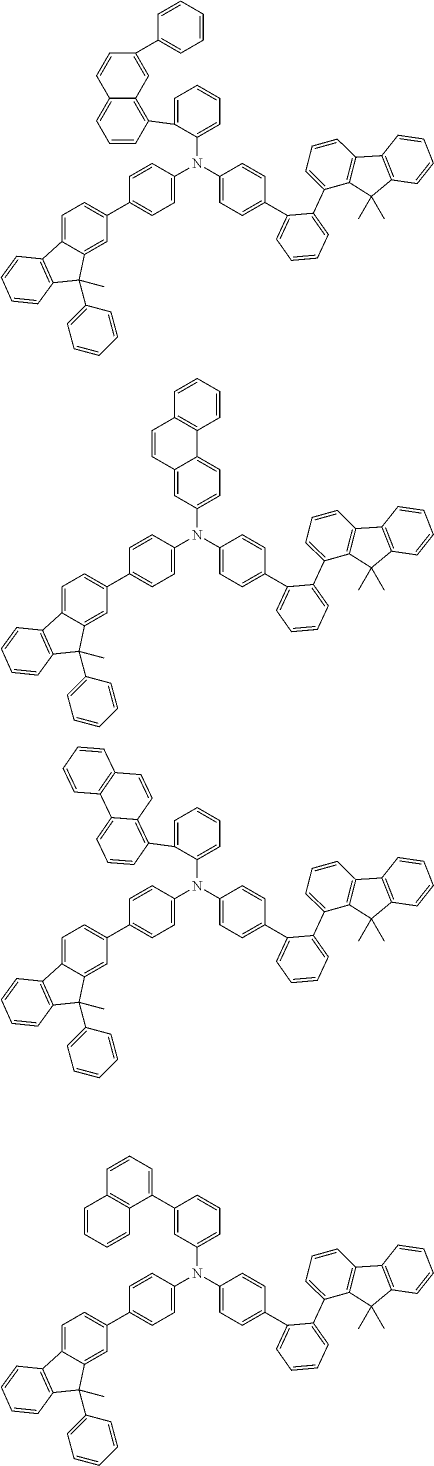

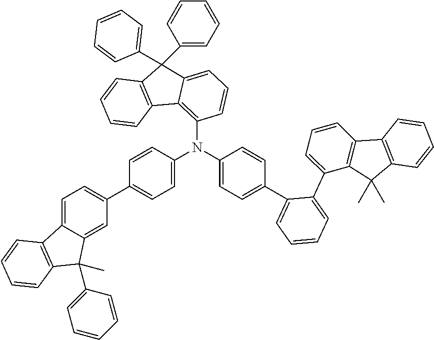 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4cccc5c4-c4ccccc4C5(c4ccccc4)c4ccccc4)cc3)c21]

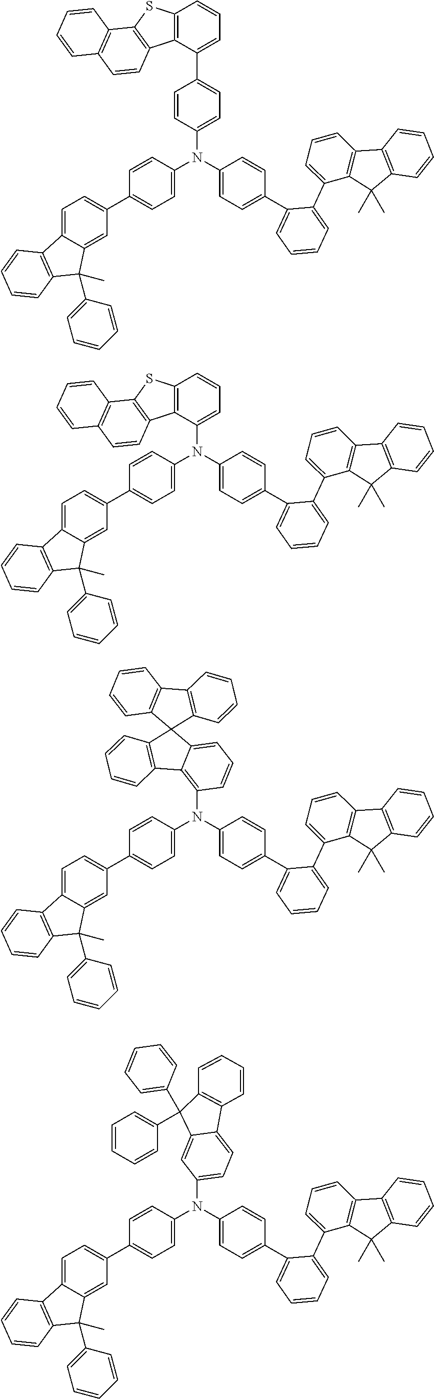

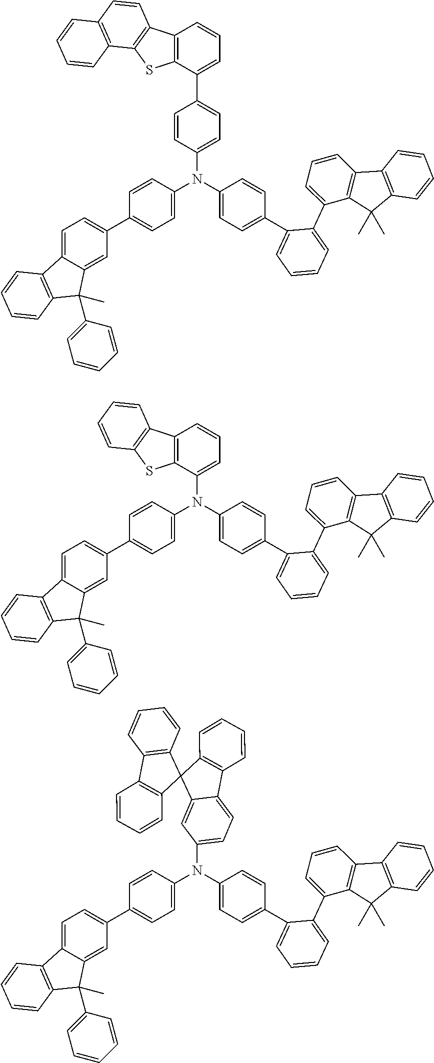

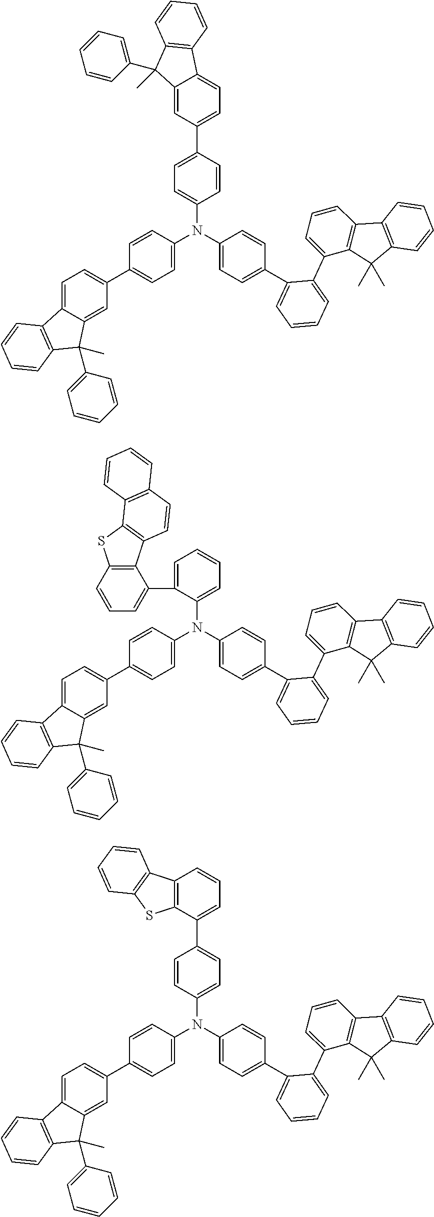

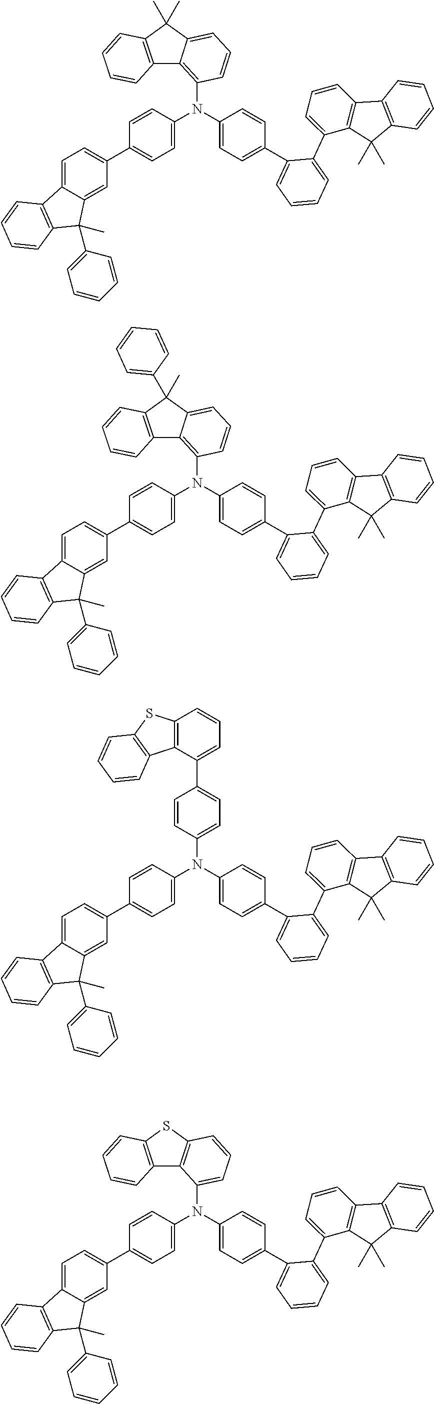 [SMILES: CC1(C)c2ccccc2-c2c(N(c3ccc(-c4ccc5c(c4)C(C)(c4ccccc4)c4ccccc4-5)cc3)c3ccc(-c4ccccc4-c4cccc5c4C(C)(C)c4ccccc4-5)cc3)cccc21 ; CC1(C)c2ccccc2-c2cccc(-c3ccccc3-c3ccc(N(c4ccc(-c5ccc6c(c5)C(C)(c5ccccc5)c5ccccc5-6)cc4)c4cccc5c4-c4ccccc4C5(C)c4ccccc4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc(-c5cccc6sc7ccccc7c56)cc4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4cccc5sc6ccccc6c45)cc3)c21]

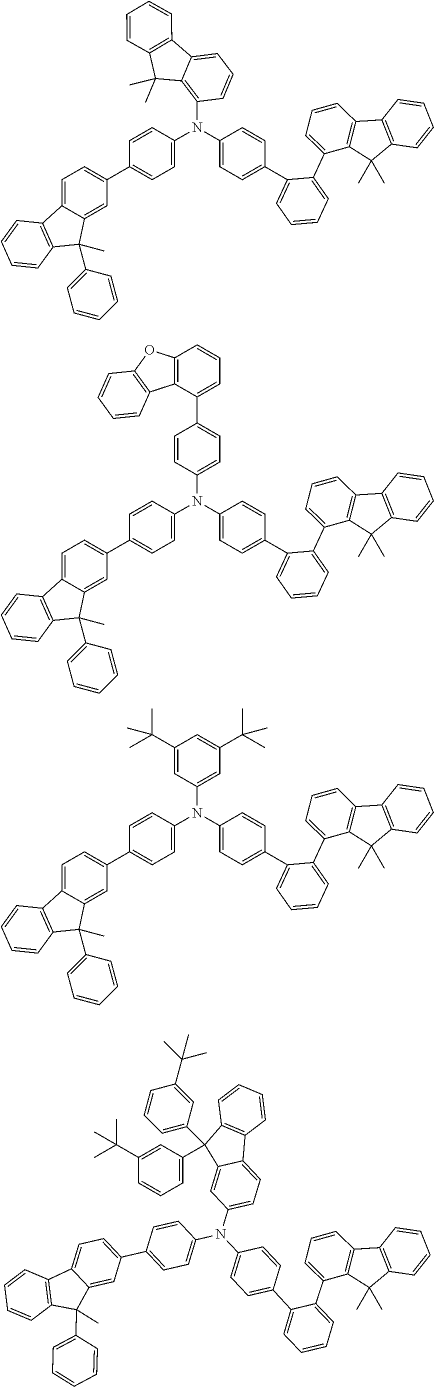

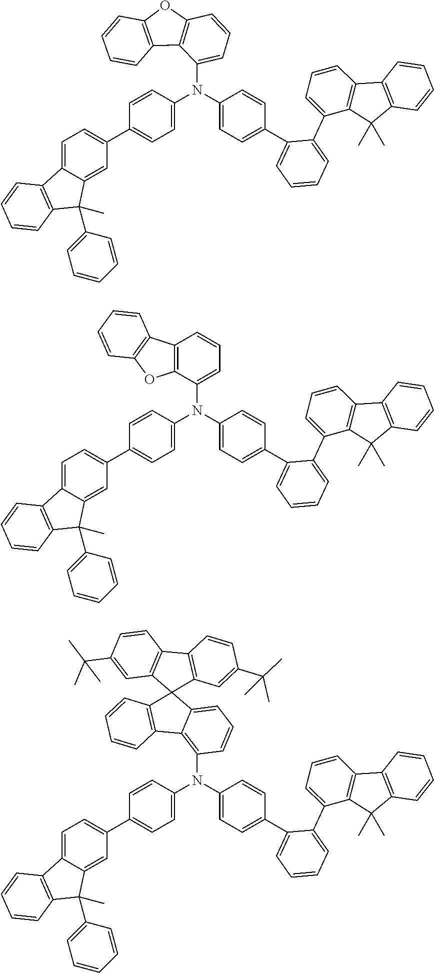

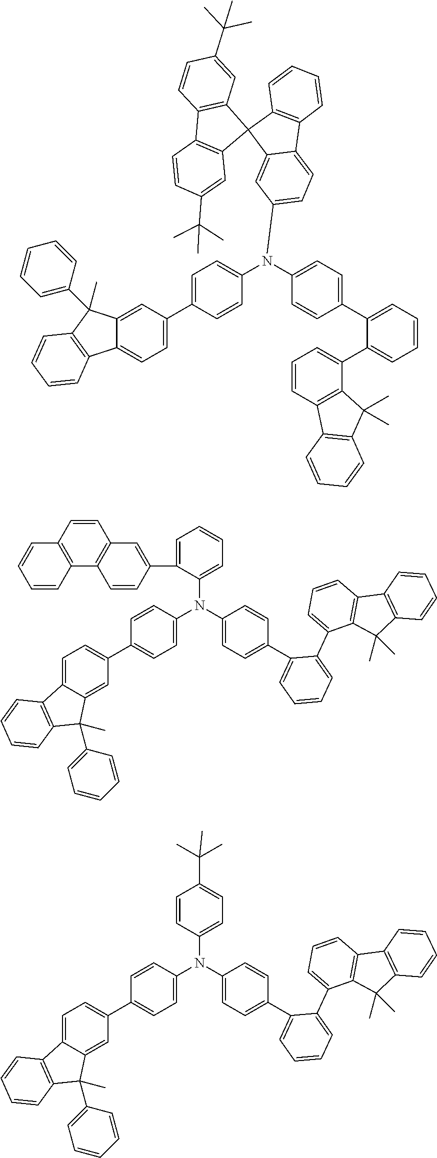 [SMILES: CC(C)(C)c1ccc2c(c1)C1(c3ccccc3-c3ccc(N(c4ccc(-c5ccc6c(c5)C(C)(c5ccccc5)c5ccccc5-6)cc4)c4ccc(-c5ccccc5-c5cccc6c5C(C)(C)c5ccccc5-6)cc4)cc31)c1cc(C(C)(C)C)ccc1-2 ; CC1(C)c2ccccc2-c2cccc(-c3ccccc3-c3ccc(N(c4ccc(-c5ccc6c(c5)C(C)(c5ccccc5)c5ccccc5-6)cc4)c4ccccc4-c4ccc5c(ccc6ccccc65)c4)cc3)c21 ; CC(C)(C)c1ccc(N(c2ccc(-c3ccc4c(c3)C(C)(c3ccccc3)c3ccccc3-4)cc2)c2ccc(-c3ccccc3-c3cccc4c3C(C)(C)c3ccccc3-4)cc2)cc1]

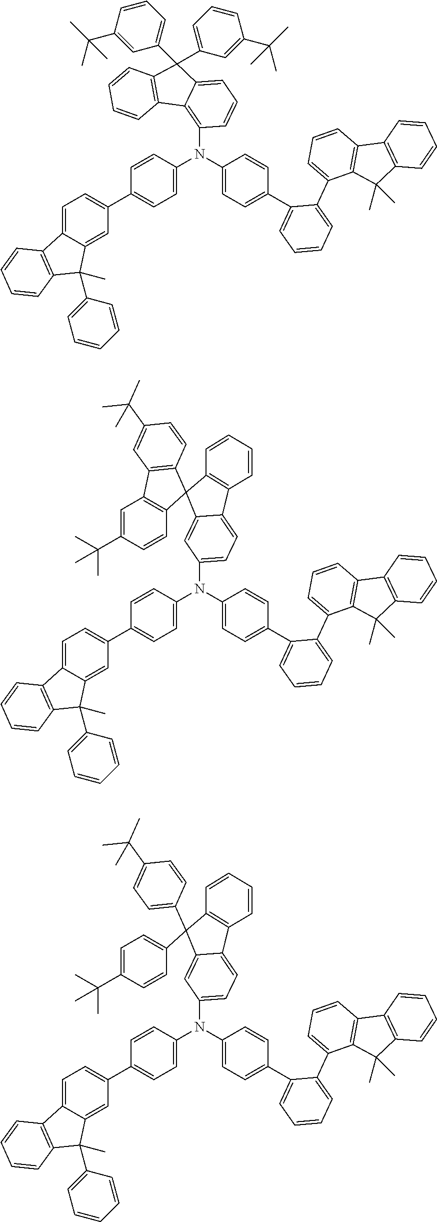 [SMILES: CC(C)(C)c1cccc(C2(c3cccc(C(C)(C)C)c3)c3ccccc3-c3c(N(c4ccc(-c5ccc6c(c5)C(C)(c5ccccc5)c5ccccc5-6)cc4)c4ccc(-c5ccccc5-c5cccc6c5C(C)(C)c5ccccc5-6)cc4)cccc32)c1 ; CC(C)(C)c1ccc2c(c1)-c1cc(C(C)(C)C)ccc1C21c2ccccc2-c2ccc(N(c3ccc(-c4ccc5c(c4)C(C)(c4ccccc4)c4ccccc4-5)cc3)c3ccc(-c4ccccc4-c4cccc5c4C(C)(C)c4ccccc4-5)cc3)cc21 ; CC(C)(C)c1ccc(C2(c3ccc(C(C)(C)C)cc3)c3ccccc3-c3ccc(N(c4ccc(-c5ccc6c(c5)C(C)(c5ccccc5)c5ccccc5-6)cc4)c4ccc(-c5ccccc5-c5cccc6c5C(C)(C)c5ccccc5-6)cc4)cc32)cc1]

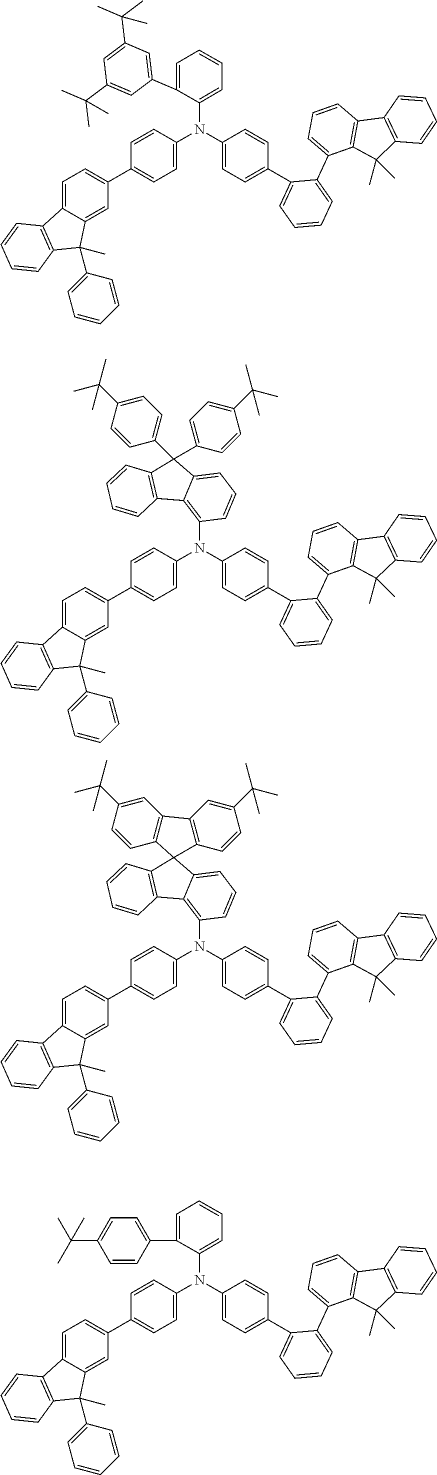

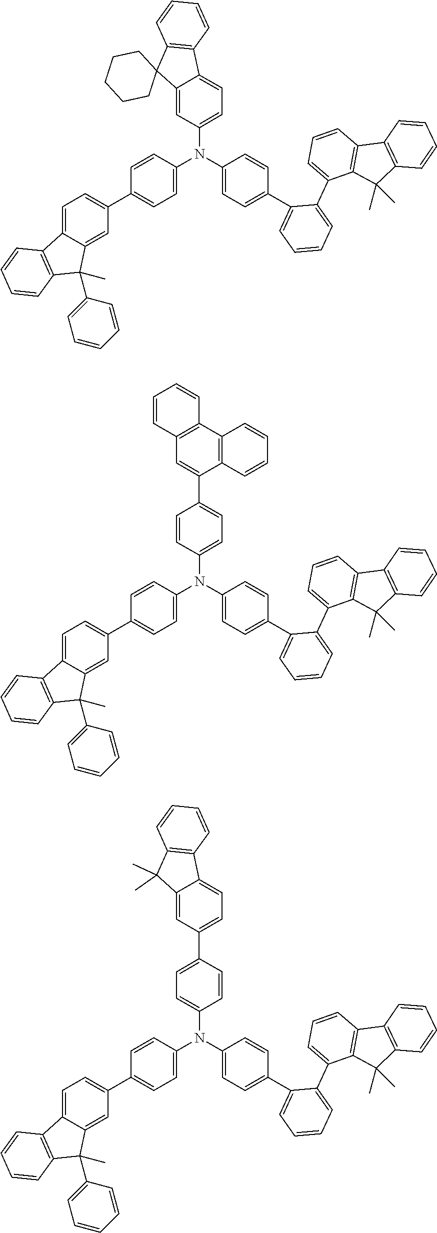 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(CCCCC4)c4ccccc4-5)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc(-c5cc6ccccc6c6ccccc56)cc4)cc3)c21 ; CC1(C)c2ccccc2-c2ccc(-c3ccc(N(c4ccc(-c5ccc6c(c5)C(C)(c5ccccc5)c5ccccc5-6)cc4)c4ccc(-c5ccccc5-c5cccc6c5C(C)(C)c5ccccc5-6)cc4)cc3)cc21]

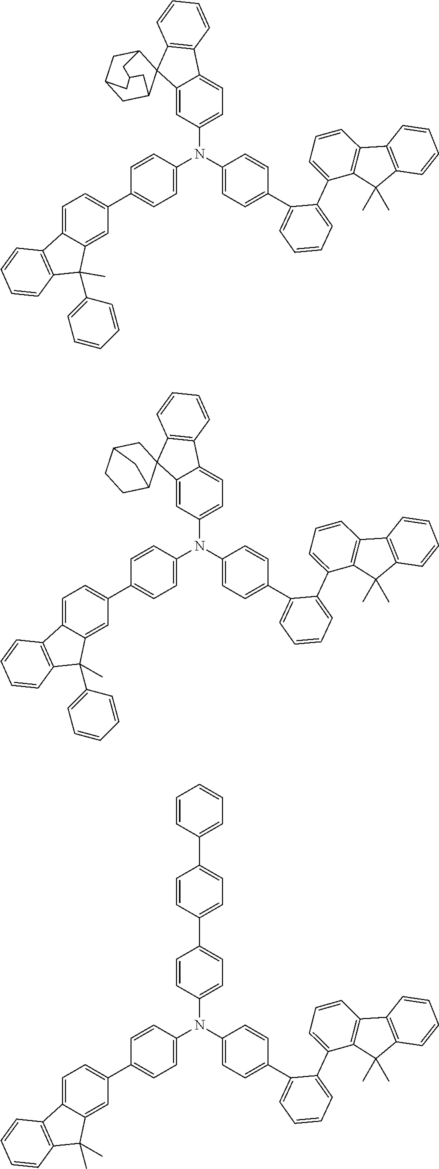 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(CC6CCC4C6)c4ccccc4-5)cc3)c21 ; CC1(C)c2ccccc2-c2ccc(-c3ccc(N(c4ccc(-c5ccc(-c6ccccc6)cc5)cc4)c4ccc(-c5ccccc5-c5cccc6c5C(C)(C)c5ccccc5-6)cc4)cc3)cc21]

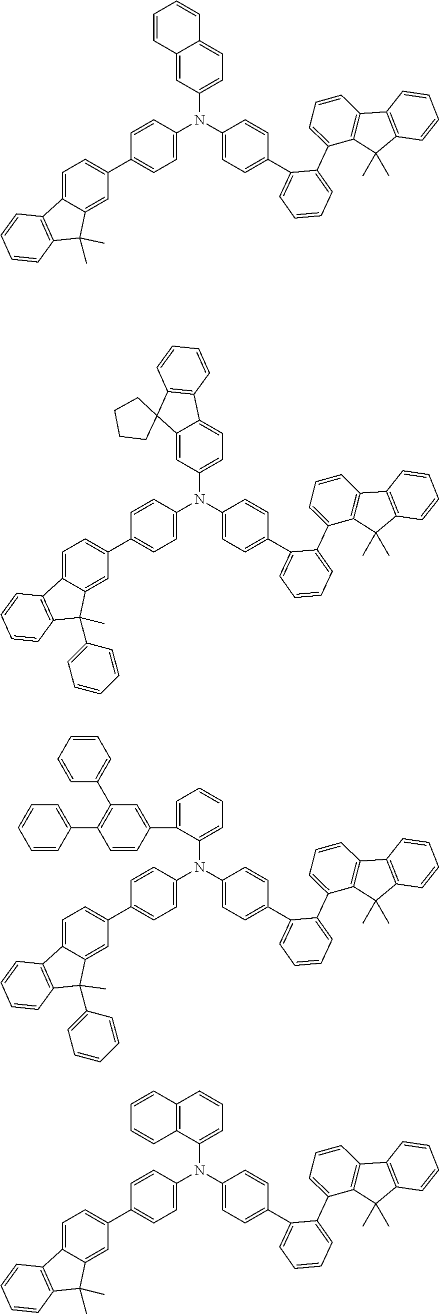 [SMILES: CC1(C)c2ccccc2-c2ccc(-c3ccc(N(c4ccc(-c5ccccc5-c5cccc6c5C(C)(C)c5ccccc5-6)cc4)c4ccc5ccccc5c4)cc3)cc21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(CCCC4)c4ccccc4-5)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccccc4-c4ccc(-c5ccccc5)c(-c5ccccc5)c4)cc3)c21 ; CC1(C)c2ccccc2-c2ccc(-c3ccc(N(c4ccc(-c5ccccc5-c5cccc6c5C(C)(C)c5ccccc5-6)cc4)c4cccc5ccccc45)cc3)cc21]

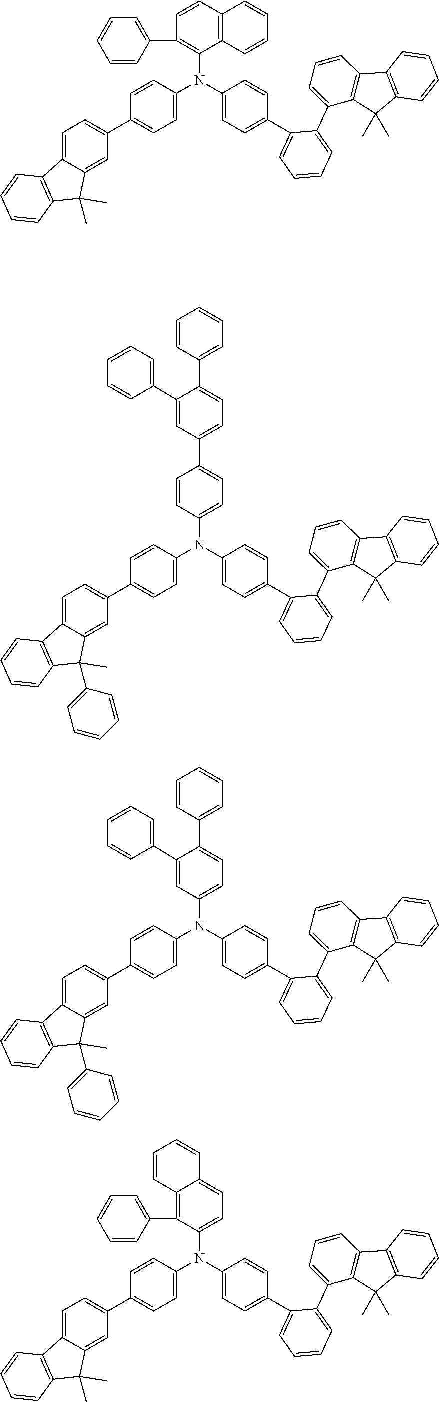 [SMILES: CC1(C)c2ccccc2-c2ccc(-c3ccc(N(c4ccc(-c5ccccc5-c5cccc6c5C(C)(C)c5ccccc5-6)cc4)c4c(-c5ccccc5)ccc5ccccc45)cc3)cc21 ; CC1(C)c2ccccc2-c2cccc(-c3ccccc3-c3ccc(N(c4ccc(-c5ccc(-c6ccccc6)c(-c6ccccc6)c5)cc4)c4ccc(-c5ccc6c(c5)C(C)(c5ccccc5)c5ccccc5-6)cc4)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc(-c5ccccc5)c(-c5ccccc5)c4)cc3)c21 ; CC1(C)c2ccccc2-c2ccc(-c3ccc(N(c4ccc(-c5ccccc5-c5cccc6c5C(C)(C)c5ccccc5-6)cc4)c4ccc5ccccc5c4-c4ccccc4)cc3)cc21]

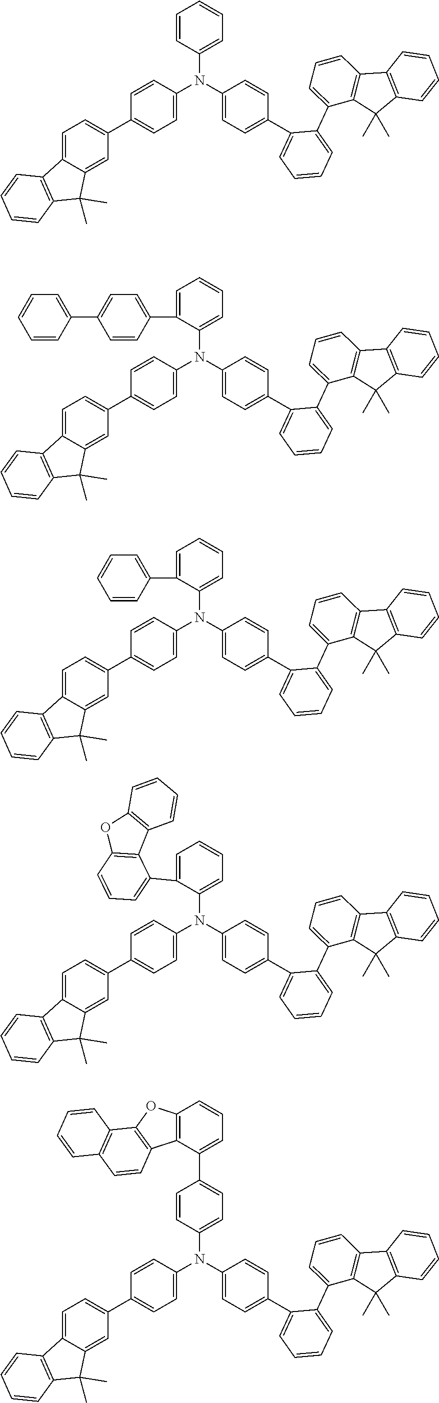

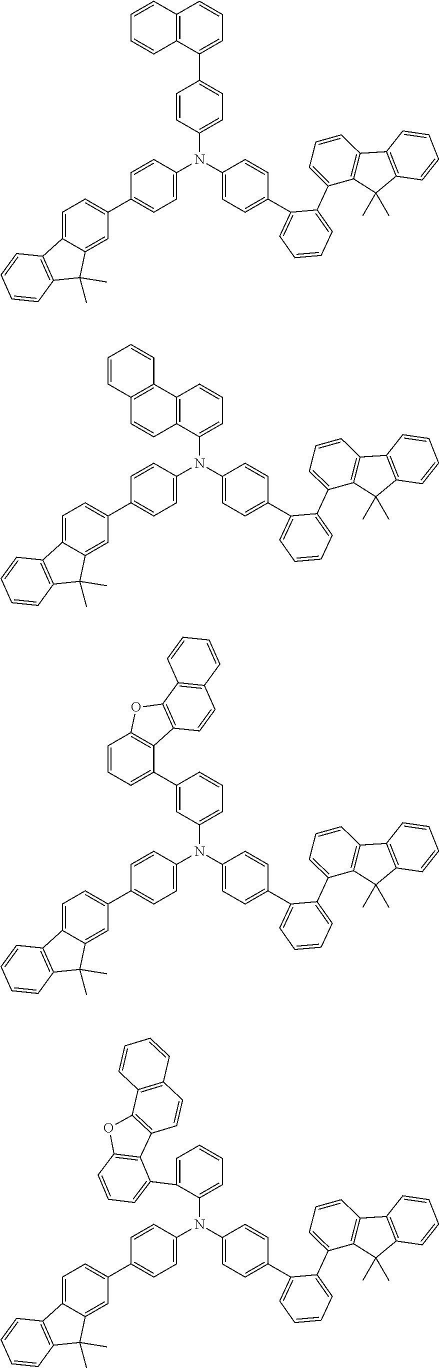 [SMILES: CC1(C)c2ccccc2-c2ccc(-c3ccc(N(c4ccc(-c5ccccc5-c5cccc6c5C(C)(C)c5ccccc5-6)cc4)c4ccc(-c5cccc6ccccc56)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5c4ccc4ccccc45)cc3)cc21 ; CC1(C)c2ccccc2-c2ccc(-c3ccc(N(c4ccc(-c5ccccc5-c5cccc6c5C(C)(C)c5ccccc5-6)cc4)c4cccc(-c5cccc6oc7c8ccccc8ccc7c56)c4)cc3)cc21 ; CC1(C)c2ccccc2-c2ccc(-c3ccc(N(c4ccc(-c5ccccc5-c5cccc6c5C(C)(C)c5ccccc5-6)cc4)c4ccccc4-c4cccc5oc6c7ccccc7ccc6c45)cc3)cc21]

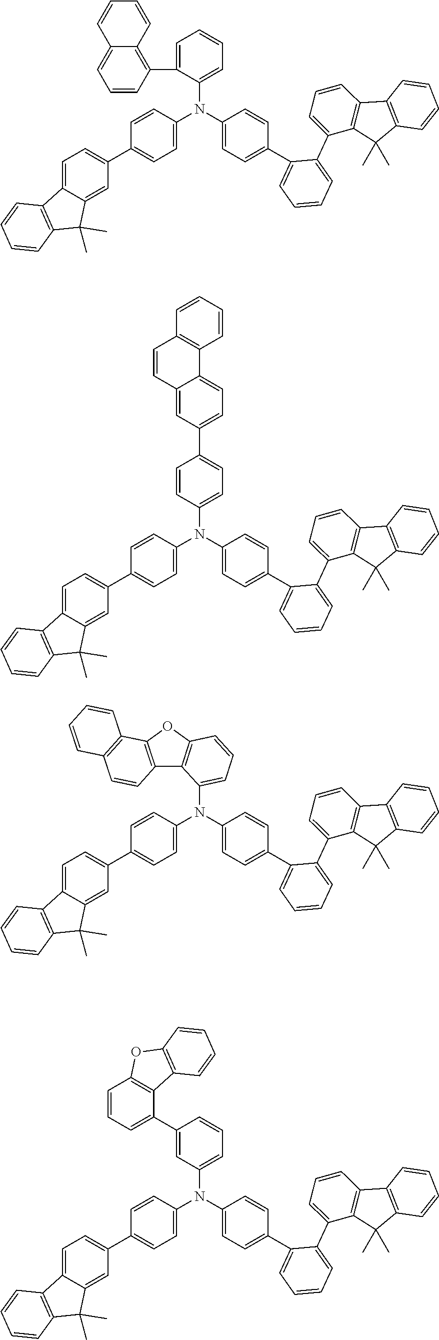

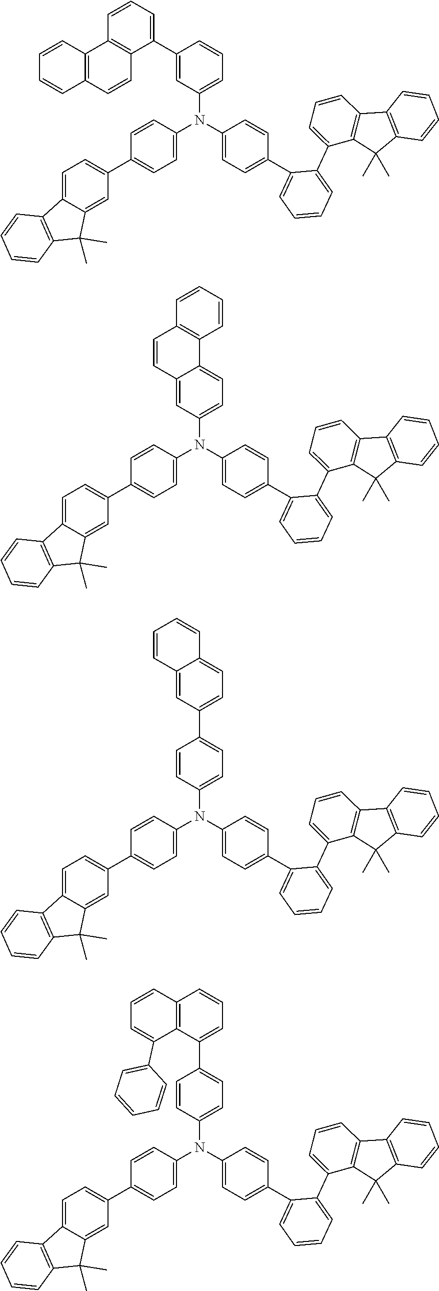 [SMILES: CC1(C)c2ccccc2-c2ccc(-c3ccc(N(c4ccc(-c5ccccc5-c5cccc6c5C(C)(C)c5ccccc5-6)cc4)c4cccc(-c5cccc6c5ccc5ccccc56)c4)cc3)cc21 ; CC1(C)c2ccccc2-c2ccc(-c3ccc(N(c4ccc(-c5ccccc5-c5cccc6c5C(C)(C)c5ccccc5-6)cc4)c4ccc5c(ccc6ccccc65)c4)cc3)cc21 ; CC1(C)c2ccccc2-c2ccc(-c3ccc(N(c4ccc(-c5ccc6ccccc6c5)cc4)c4ccc(-c5ccccc5-c5cccc6c5C(C)(C)c5ccccc5-6)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4ccc(-c5cccc6cccc(-c7ccccc7)c56)cc4)cc3)cc21]

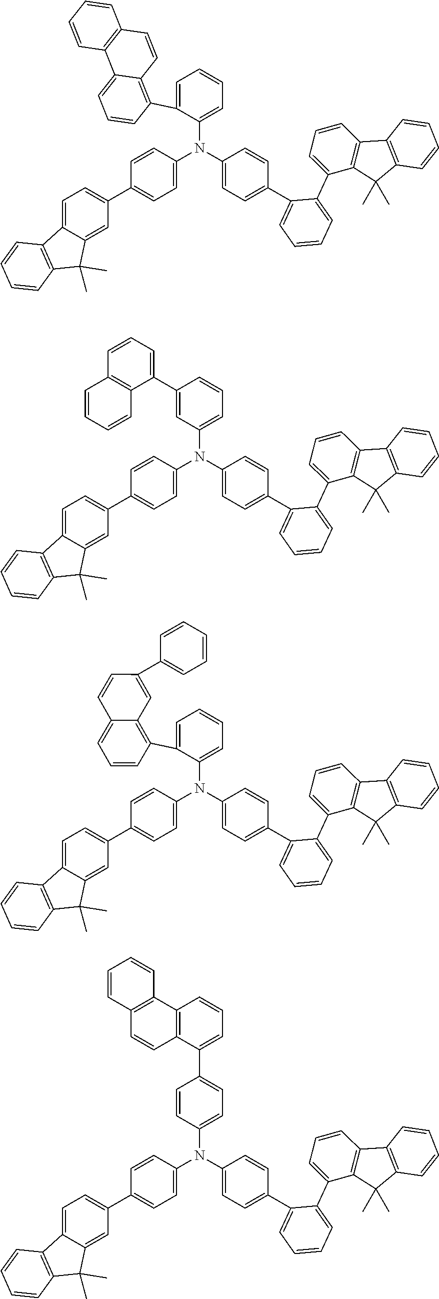 [SMILES: CC1(C)c2ccccc2-c2ccc(-c3ccc(N(c4ccc(-c5ccccc5-c5cccc6c5C(C)(C)c5ccccc5-6)cc4)c4ccccc4-c4cccc5c4ccc4ccccc45)cc3)cc21 ; CC1(C)c2ccccc2-c2ccc(-c3ccc(N(c4ccc(-c5ccccc5-c5cccc6c5C(C)(C)c5ccccc5-6)cc4)c4cccc(-c5cccc6ccccc56)c4)cc3)cc21 ; CC1(C)c2ccccc2-c2ccc(-c3ccc(N(c4ccc(-c5ccccc5-c5cccc6c5C(C)(C)c5ccccc5-6)cc4)c4ccccc4-c4cccc5ccc(-c6ccccc6)cc45)cc3)cc21 ; CC1(C)c2ccccc2-c2ccc(-c3ccc(N(c4ccc(-c5ccccc5-c5cccc6c5C(C)(C)c5ccccc5-6)cc4)c4ccc(-c5cccc6c5ccc5ccccc56)cc4)cc3)cc21]

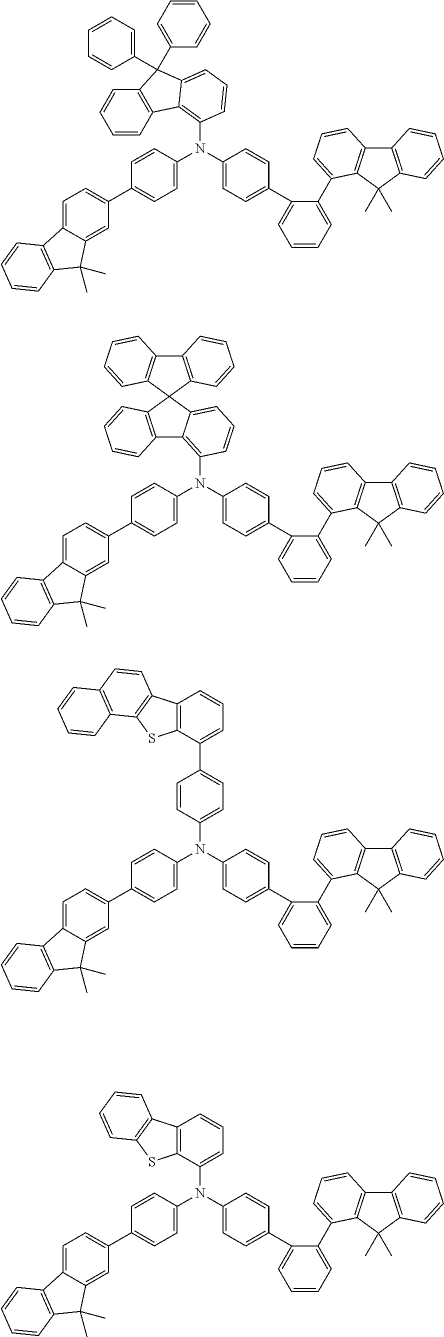 [SMILES: CC1(C)c2ccccc2-c2ccc(-c3ccc(N(c4ccc(-c5ccccc5-c5cccc6c5C(C)(C)c5ccccc5-6)cc4)c4cccc5c4-c4ccccc4C5(c4ccccc4)c4ccccc4)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5c4-c4ccccc4C54c5ccccc5-c5ccccc54)cc3)cc21 ; CC1(C)c2ccccc2-c2ccc(-c3ccc(N(c4ccc(-c5ccccc5-c5cccc6c5C(C)(C)c5ccccc5-6)cc4)c4ccc(-c5cccc6c5sc5c7ccccc7ccc65)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5c4sc4ccccc45)cc3)cc21]

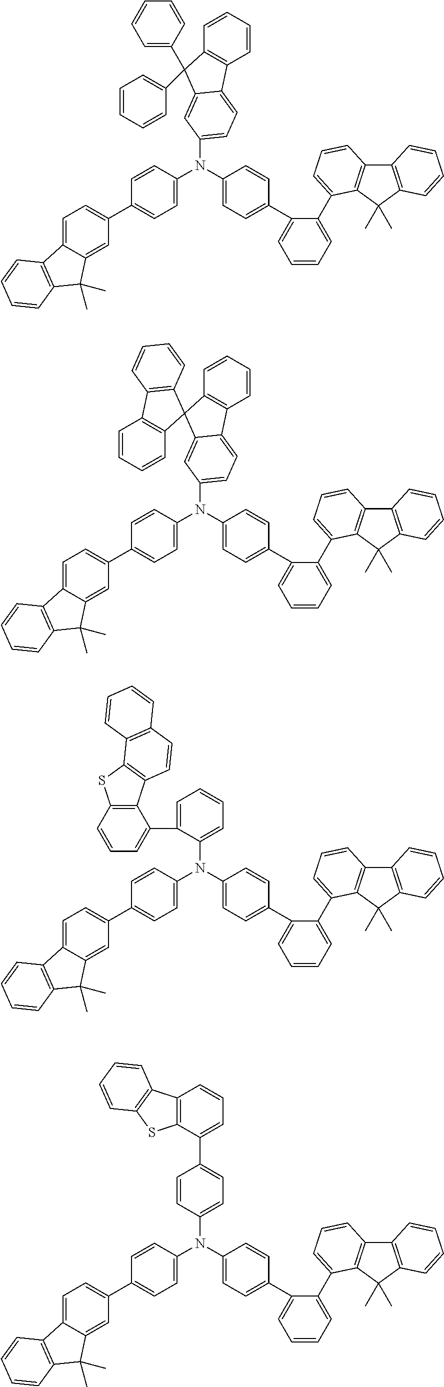

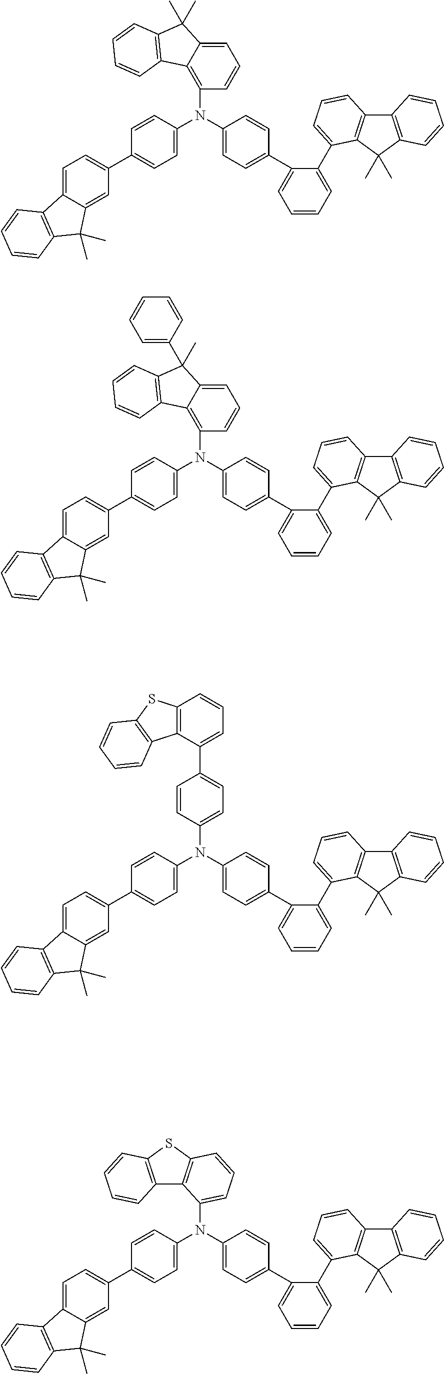 [SMILES: CC1(C)c2ccccc2-c2ccc(-c3ccc(N(c4ccc(-c5ccccc5-c5cccc6c5C(C)(C)c5ccccc5-6)cc4)c4cccc5c4-c4ccccc4C5(C)C)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5c4-c4ccccc4C5(C)c4ccccc4)cc3)cc21 ; CC1(C)c2ccccc2-c2ccc(-c3ccc(N(c4ccc(-c5ccccc5-c5cccc6c5C(C)(C)c5ccccc5-6)cc4)c4ccc(-c5cccc6sc7ccccc7c56)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5sc6ccccc6c45)cc3)cc21]

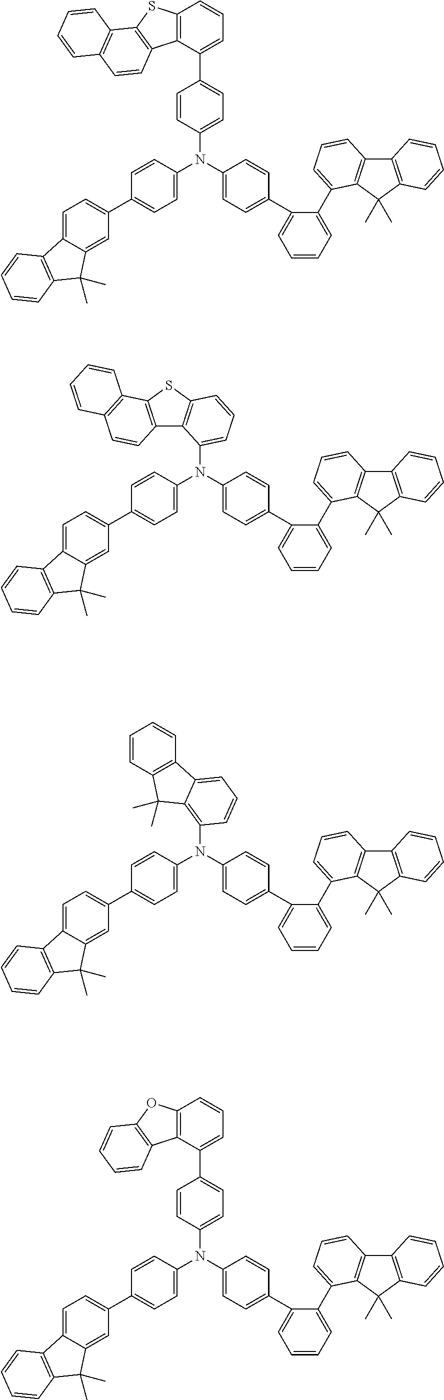 [SMILES: CC1(C)c2ccccc2-c2ccc(-c3ccc(N(c4ccc(-c5ccccc5-c5cccc6c5C(C)(C)c5ccccc5-6)cc4)c4ccc(-c5cccc6sc7c8ccccc8ccc7c56)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5sc6c7ccccc7ccc6c45)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5c4C(C)(C)c4ccccc4-5)cc3)cc21 ; CC1(C)c2ccccc2-c2ccc(-c3ccc(N(c4ccc(-c5ccccc5-c5cccc6c5C(C)(C)c5ccccc5-6)cc4)c4ccc(-c5cccc6oc7ccccc7c56)cc4)cc3)cc21]

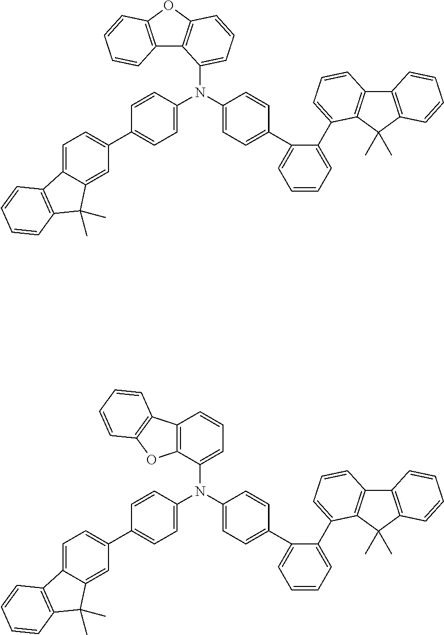 [SMILES: CC1(C)c2ccccc2-c2ccc(-c3ccc(N(c4ccc(-c5ccccc5-c5cccc6c5C(C)(C)c5ccccc5-6)cc4)c4cccc5oc6ccccc6c45)cc3)cc21 ; CC1(C)c2ccccc2-c2ccc(-c3ccc(N(c4ccc(-c5ccccc5-c5cccc6c5C(C)(C)c5ccccc5-6)cc4)c4cccc5c4oc4ccccc45)cc3)cc21]

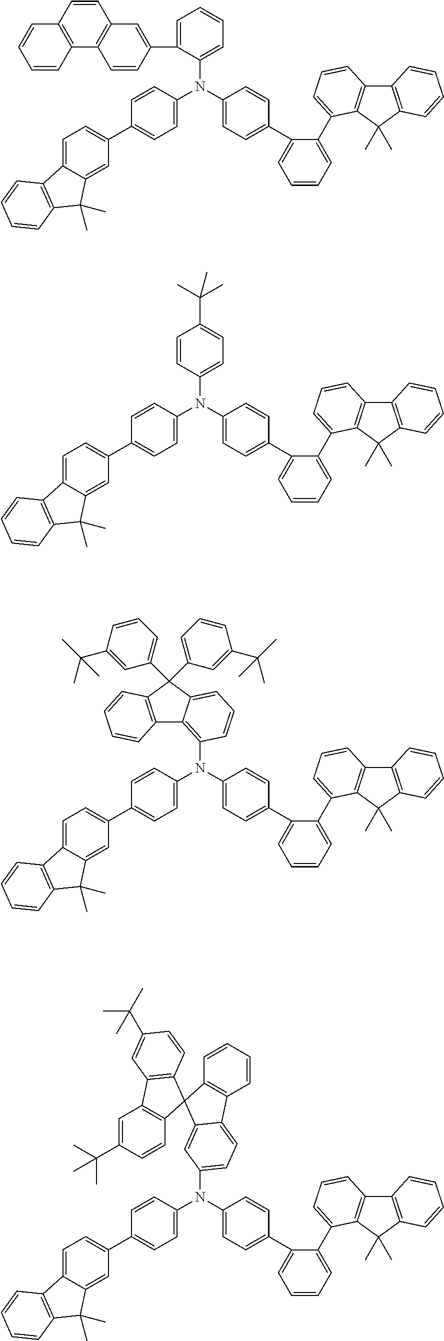

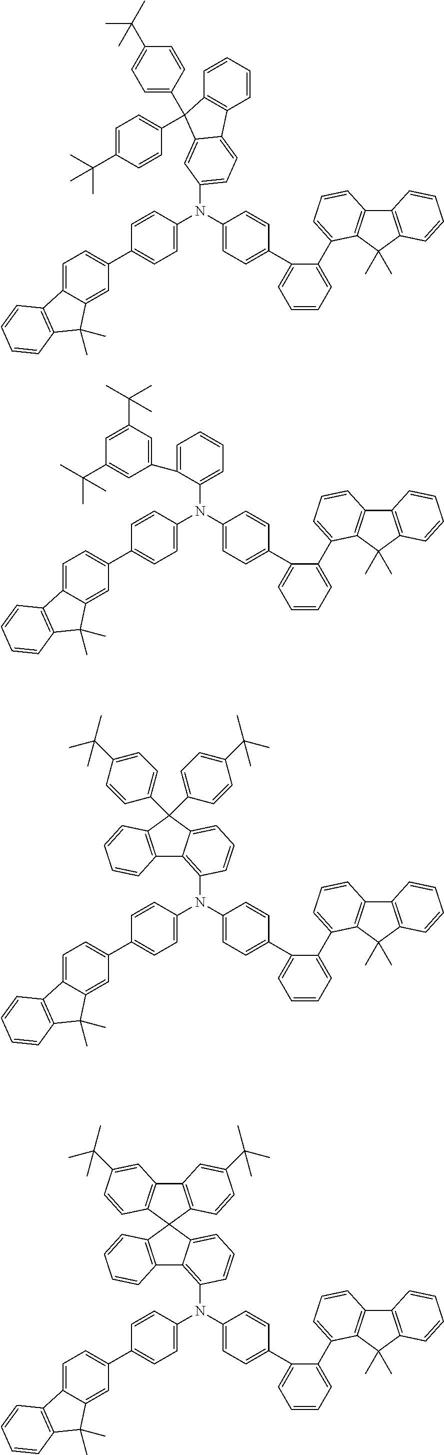

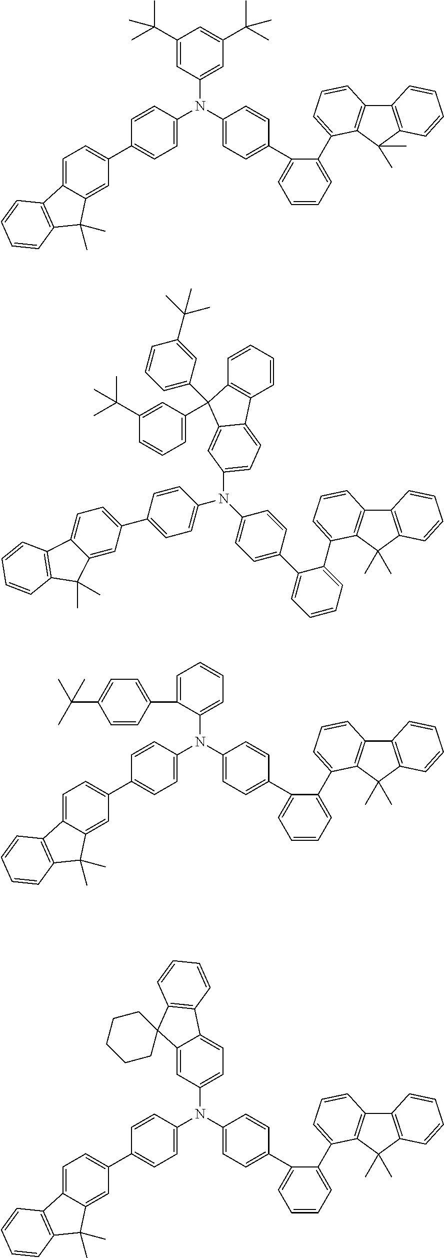

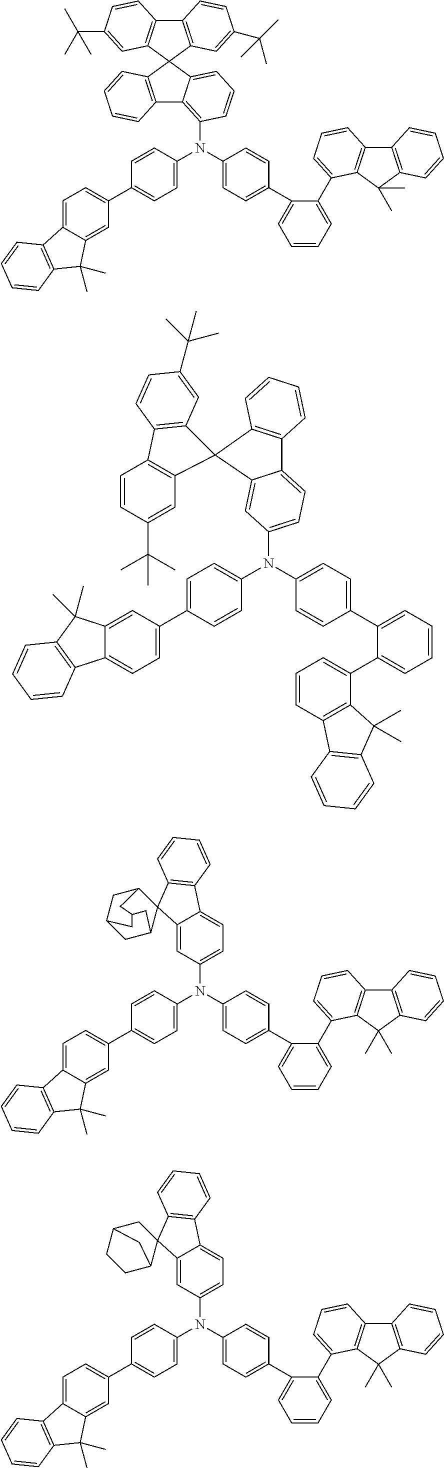

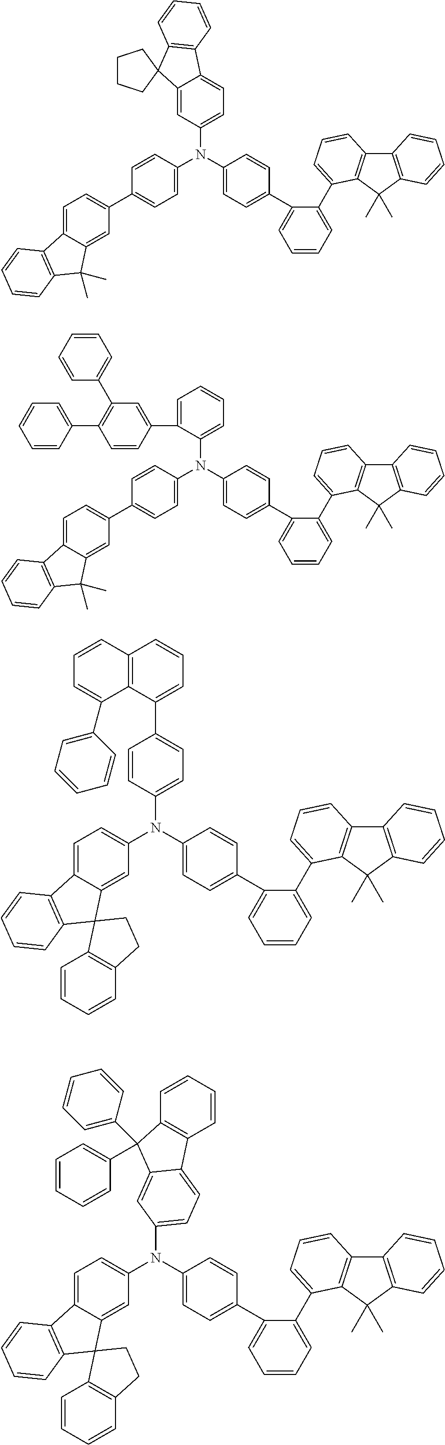

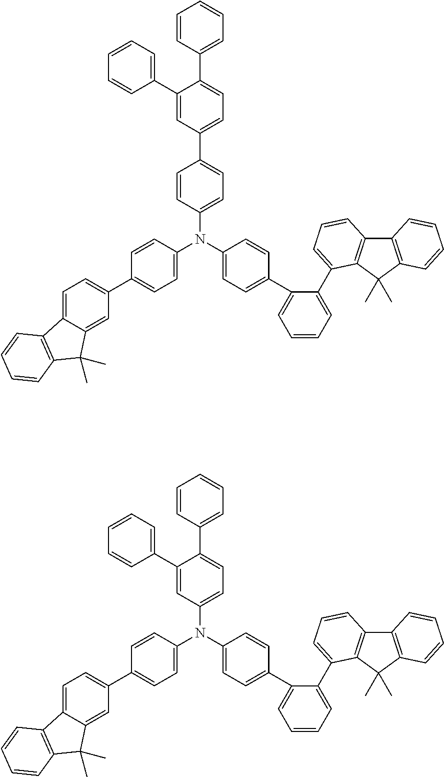 [SMILES: CC1(C)c2ccccc2-c2ccc(-c3ccc(N(c4ccc(-c5ccc(-c6ccccc6)c(-c6ccccc6)c5)cc4)c4ccc(-c5ccccc5-c5cccc6c5C(C)(C)c5ccccc5-6)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4ccc(-c5ccccc5)c(-c5ccccc5)c4)cc3)cc21]

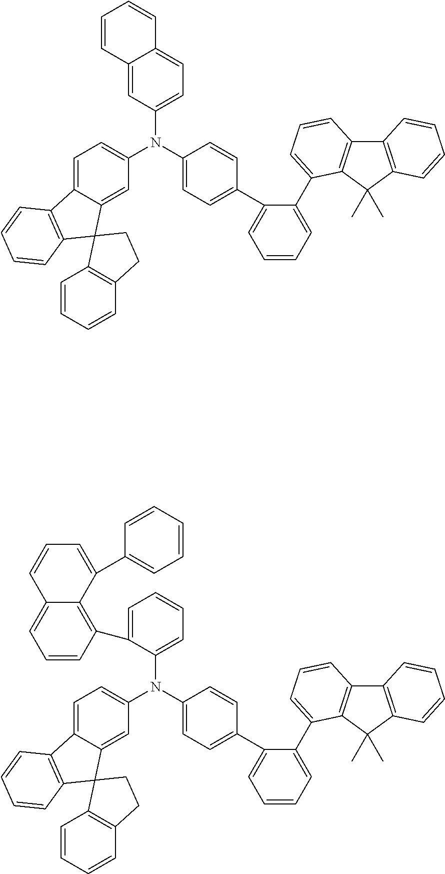 [SMILES: CC1(C)c2ccccc2-c2cccc(-c3ccccc3-c3ccc(N(c4ccc5c(c4)C4(CCc6ccccc64)c4ccccc4-5)c4ccc5ccccc5c4)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4cccc5cccc(-c6ccccc6)c45)cc3)c21]

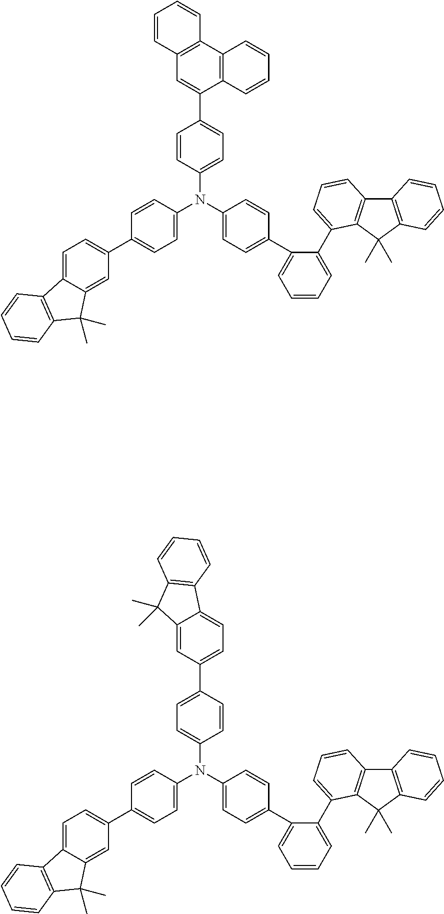 [SMILES: CC1(C)c2ccccc2-c2ccc(-c3ccc(N(c4ccc(-c5ccccc5-c5cccc6c5C(C)(C)c5ccccc5-6)cc4)c4ccc(-c5cc6ccccc6c6ccccc56)cc4)cc3)cc21 ; CC1(C)c2ccccc2-c2ccc(-c3ccc(N(c4ccc(-c5ccc6c(c5)C(C)(C)c5ccccc5-6)cc4)c4ccc(-c5ccccc5-c5cccc6c5C(C)(C)c5ccccc5-6)cc4)cc3)cc21]

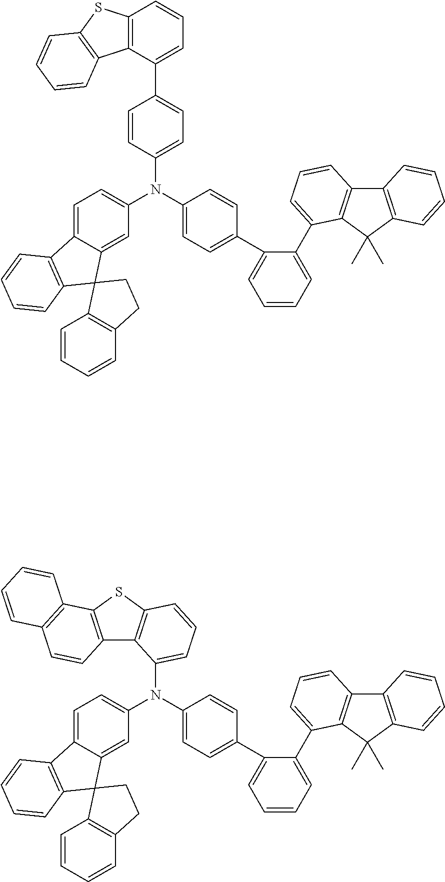 [SMILES: CC1(C)c2ccccc2-c2cccc(-c3ccccc3-c3ccc(N(c4ccc(-c5cccc6sc7ccccc7c56)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4cccc5sc6c7ccccc7ccc6c45)cc3)c21]

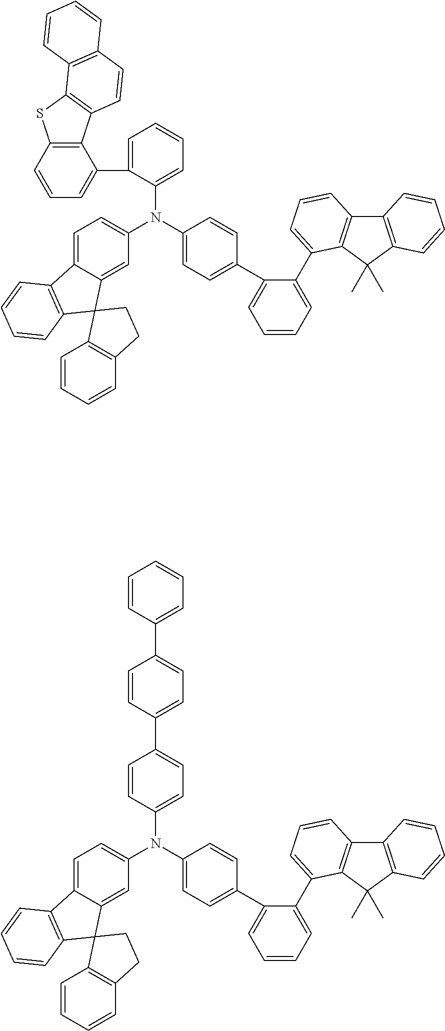 [SMILES: CC1(C)c2ccccc2-c2cccc(-c3ccccc3-c3ccc(N(c4ccc5c(c4)C4(CCc6ccccc64)c4ccccc4-5)c4ccccc4-c4cccc5sc6c7ccccc7ccc6c45)cc3)c21 ; CC1(C)c2ccccc2-c2cccc(-c3ccccc3-c3ccc(N(c4ccc(-c5ccc(-c6ccccc6)cc5)cc4)c4ccc5c(c4)C4(CCc6ccccc64)c4ccccc4-5)cc3)c21]

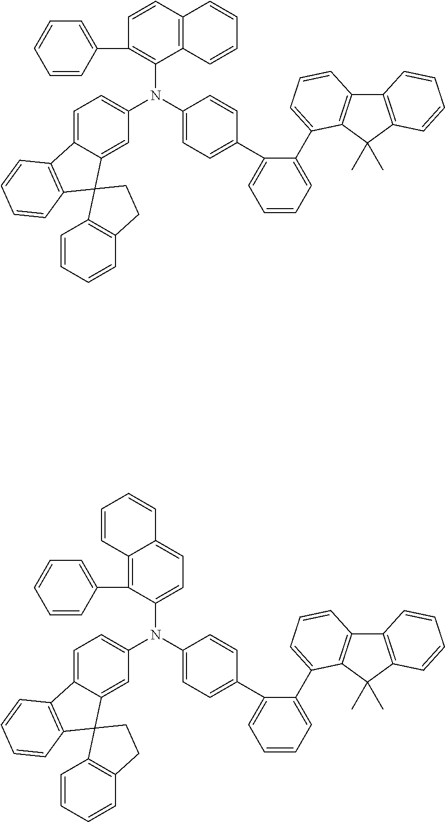 [SMILES: CC1(C)c2ccccc2-c2cccc(-c3ccccc3-c3ccc(N(c4ccc5c(c4)C4(CCc6ccccc64)c4ccccc4-5)c4c(-c5ccccc5)ccc5ccccc45)cc3)c21 ; CC1(C)c2ccccc2-c2cccc(-c3ccccc3-c3ccc(N(c4ccc5c(c4)C4(CCc6ccccc64)c4ccccc4-5)c4ccc5ccccc5c4-c4ccccc4)cc3)c21]

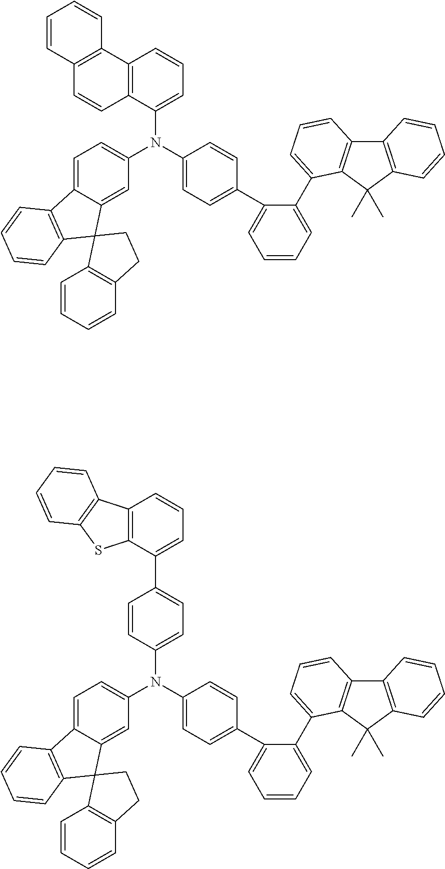 [SMILES: CC1(C)c2ccccc2-c2cccc(-c3ccccc3-c3ccc(N(c4ccc5c(c4)C4(CCc6ccccc64)c4ccccc4-5)c4cccc5c4ccc4ccccc45)cc3)c21 ; CC1(C)c2ccccc2-c2cccc(-c3ccccc3-c3ccc(N(c4ccc(-c5cccc6c5sc5ccccc56)cc4)c4ccc5c(c4)C4(CCc6ccccc64)c4ccccc4-5)cc3)c21]

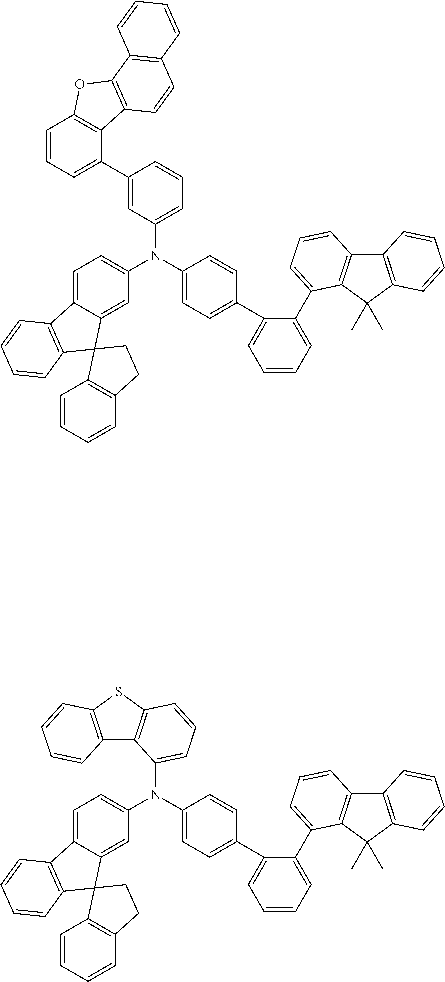 [SMILES: CC1(C)c2ccccc2-c2cccc(-c3ccccc3-c3ccc(N(c4cccc(-c5cccc6oc7c8ccccc8ccc7c56)c4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4cccc5sc6ccccc6c45)cc3)c21]

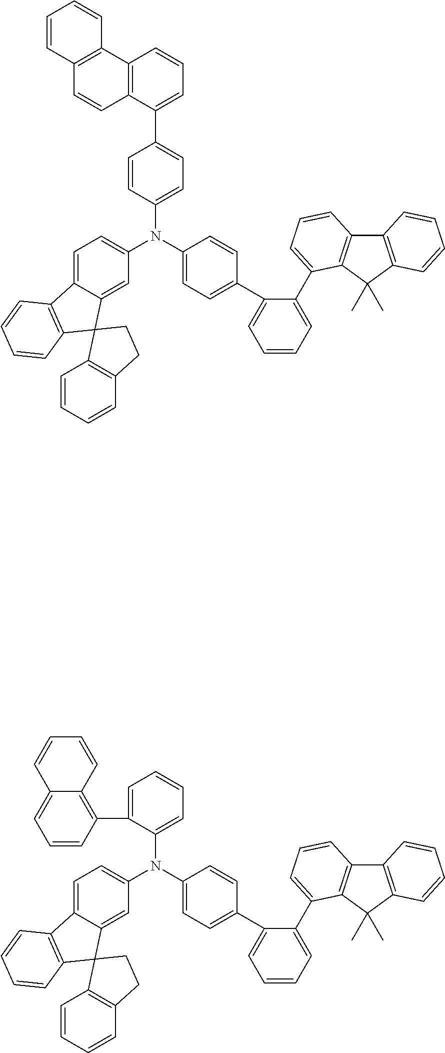 [SMILES: CC1(C)c2ccccc2-c2cccc(-c3ccccc3-c3ccc(N(c4ccc(-c5cccc6c5ccc5ccccc56)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4cccc5ccccc45)cc3)c21]

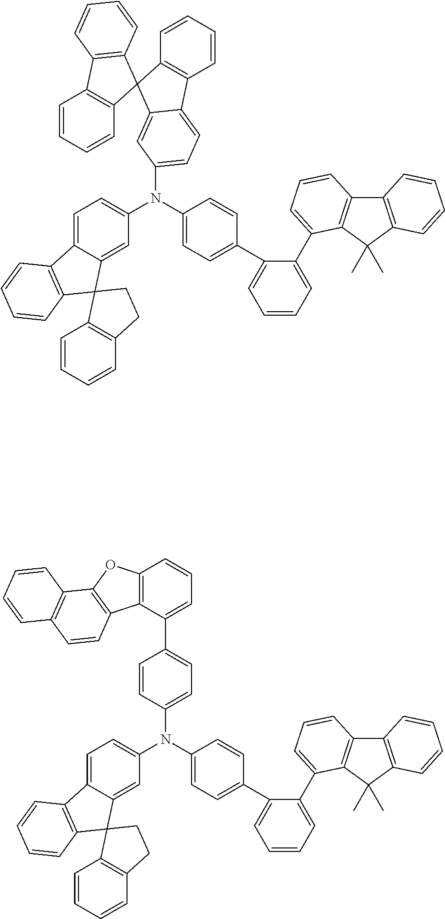 [SMILES: CC1(C)c2ccccc2-c2cccc(-c3ccccc3-c3ccc(N(c4ccc5c(c4)C4(CCc6ccccc64)c4ccccc4-5)c4ccc5c(c4)C4(c6ccccc6-c6ccccc64)c4ccccc4-5)cc3)c21 ; CC1(C)c2ccccc2-c2cccc(-c3ccccc3-c3ccc(N(c4ccc(-c5cccc6oc7c8ccccc8ccc7c56)cc4)c4ccc5c(c4)C4(CCc6ccccc64)c4ccccc4-5)cc3)c21]

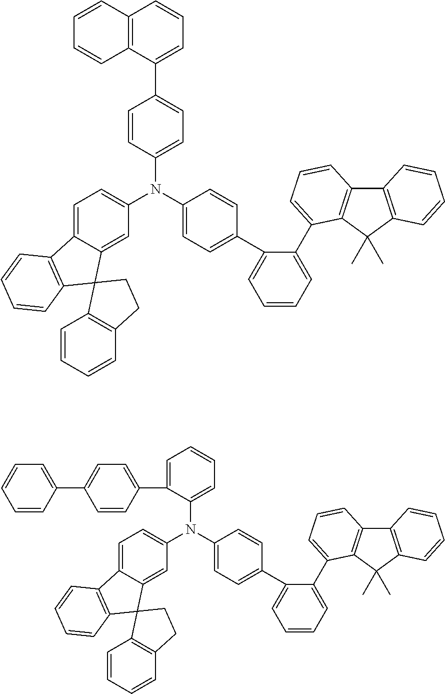 [SMILES: CC1(C)c2ccccc2-c2cccc(-c3ccccc3-c3ccc(N(c4ccc(-c5cccc6ccccc56)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4ccc(-c5ccccc5)cc4)cc3)c21]

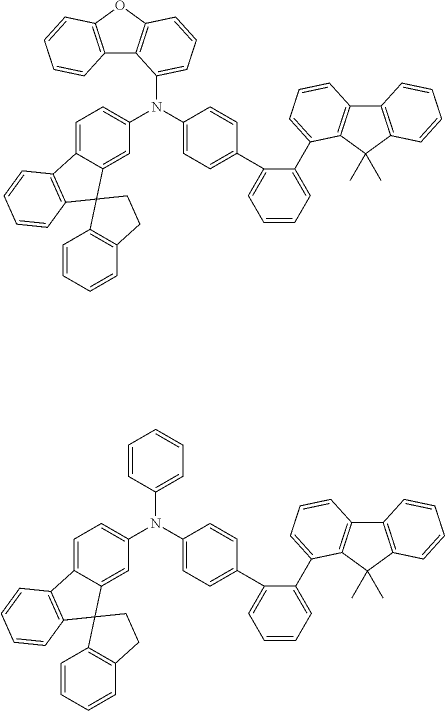 [SMILES: CC1(C)c2ccccc2-c2cccc(-c3ccccc3-c3ccc(N(c4ccc5c(c4)C4(CCc6ccccc64)c4ccccc4-5)c4cccc5oc6ccccc6c45)cc3)c21 ; CC1(C)c2ccccc2-c2cccc(-c3ccccc3-c3ccc(N(c4ccccc4)c4ccc5c(c4)C4(CCc6ccccc64)c4ccccc4-5)cc3)c21]

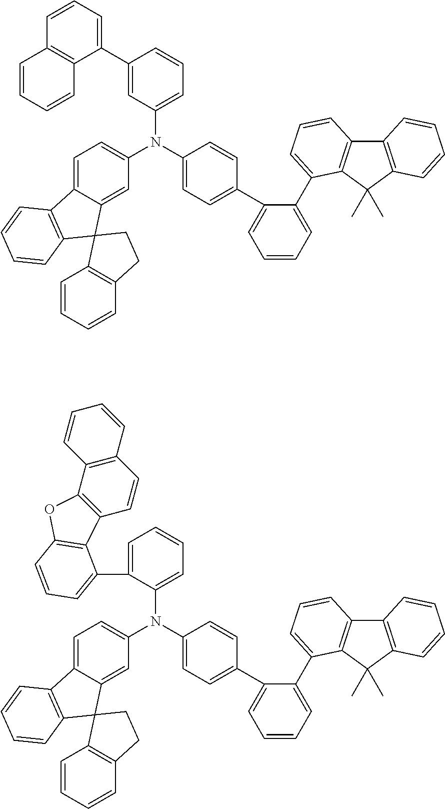 [SMILES: CC1(C)c2ccccc2-c2cccc(-c3ccccc3-c3ccc(N(c4cccc(-c5cccc6ccccc56)c4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4cccc5oc6c7ccccc7ccc6c45)cc3)c21]

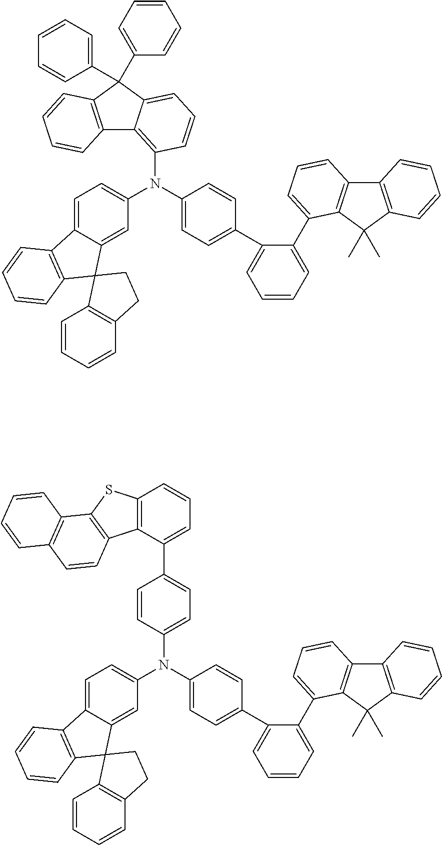 [SMILES: CC1(C)c2ccccc2-c2cccc(-c3ccccc3-c3ccc(N(c4ccc5c(c4)C4(CCc6ccccc64)c4ccccc4-5)c4cccc5c4-c4ccccc4C5(c4ccccc4)c4ccccc4)cc3)c21 ; CC1(C)c2ccccc2-c2cccc(-c3ccccc3-c3ccc(N(c4ccc(-c5cccc6sc7c8ccccc8ccc7c56)cc4)c4ccc5c(c4)C4(CCc6ccccc64)c4ccccc4-5)cc3)c21]

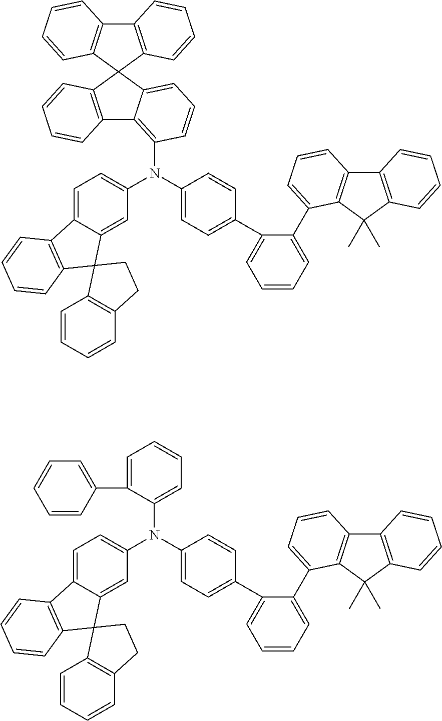 [SMILES: CC1(C)c2ccccc2-c2cccc(-c3ccccc3-c3ccc(N(c4ccc5c(c4)C4(CCc6ccccc64)c4ccccc4-5)c4cccc5c4-c4ccccc4C54c5ccccc5-c5ccccc54)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4ccccc4)cc3)c21]

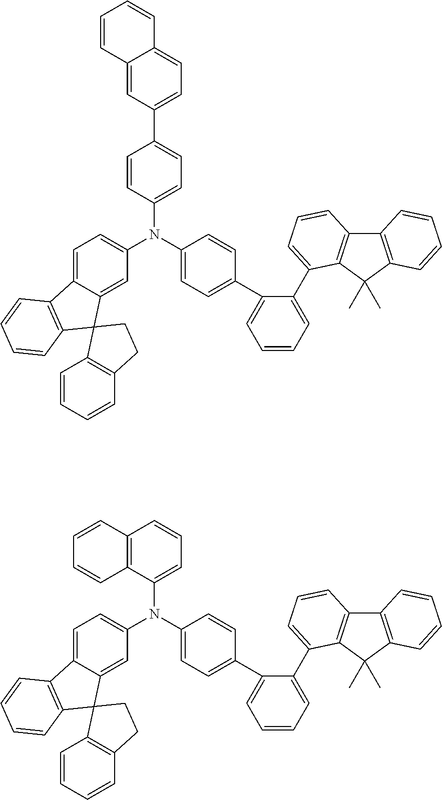 [SMILES: CC1(C)c2ccccc2-c2cccc(-c3ccccc3-c3ccc(N(c4ccc(-c5ccc6ccccc6c5)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4cccc5ccccc45)cc3)c21]

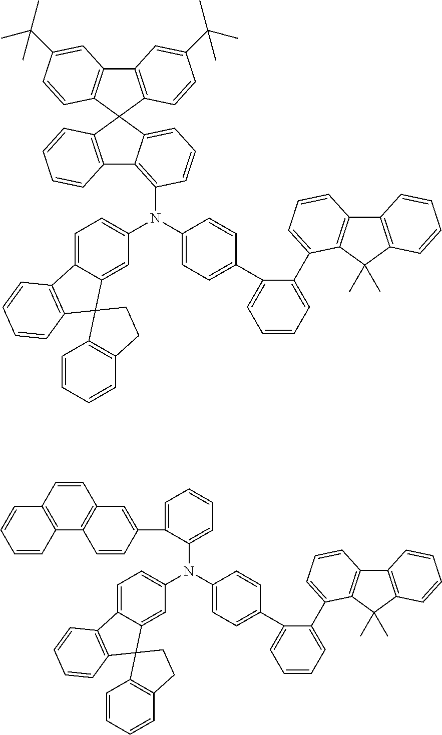 [SMILES: CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C3(CCc5ccccc53)c3ccccc3-4)cccc21 ; CC1(C)c2ccccc2-c2cccc(-c3ccccc3-c3ccc(N(c4ccc5c(c4)C4(CCc6ccccc64)c4ccccc4-5)c4ccccc4-c4ccc5c(ccc6ccccc65)c4)cc3)c21]

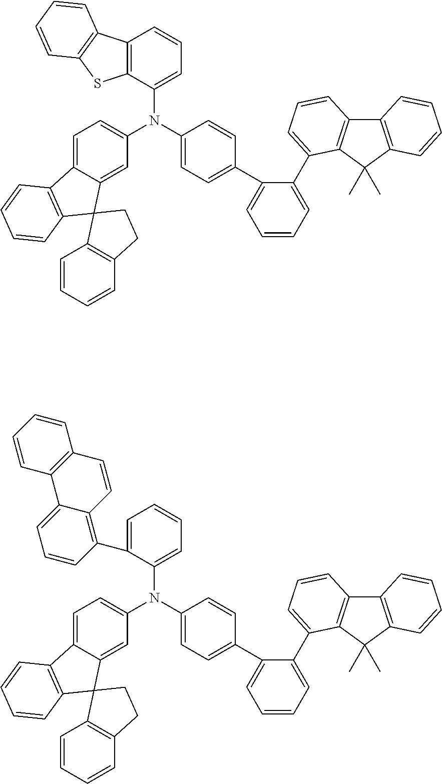 [SMILES: CC1(C)c2ccccc2-c2cccc(-c3ccccc3-c3ccc(N(c4ccc5c(c4)C4(CCc6ccccc64)c4ccccc4-5)c4cccc5c4sc4ccccc45)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4cccc5c4ccc4ccccc45)cc3)c21]

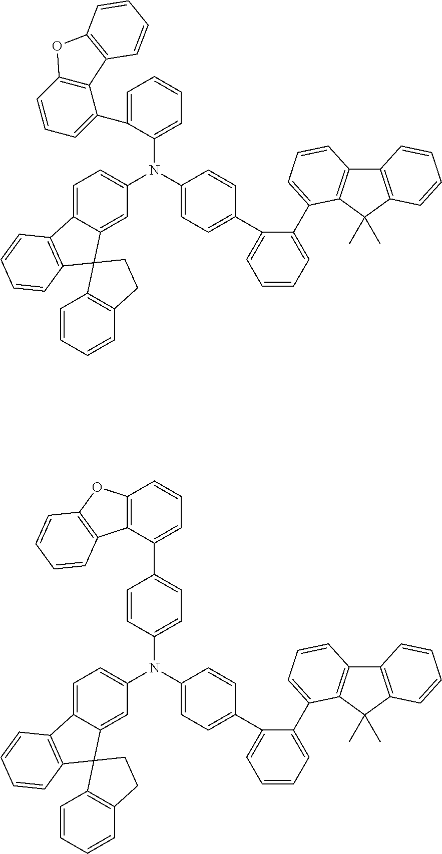 [SMILES: CC1(C)c2ccccc2-c2cccc(-c3ccccc3-c3ccc(N(c4ccc5c(c4)C4(CCc6ccccc64)c4ccccc4-5)c4ccccc4-c4cccc5oc6ccccc6c45)cc3)c21 ; CC1(C)c2ccccc2-c2cccc(-c3ccccc3-c3ccc(N(c4ccc(-c5cccc6oc7ccccc7c56)cc4)c4ccc5c(c4)C4(CCc6ccccc64)c4ccccc4-5)cc3)c21]

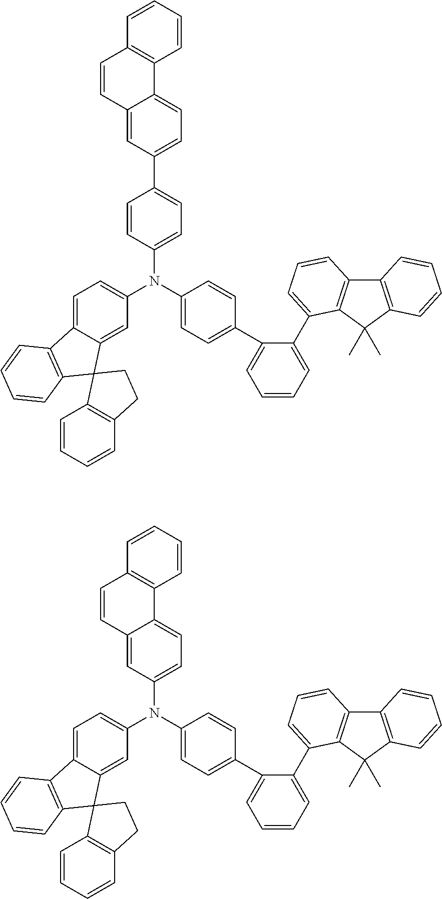 [SMILES: CC1(C)c2ccccc2-c2cccc(-c3ccccc3-c3ccc(N(c4ccc(-c5ccc6c(ccc7ccccc76)c5)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4ccc5c(ccc6ccccc65)c4)cc3)c21]

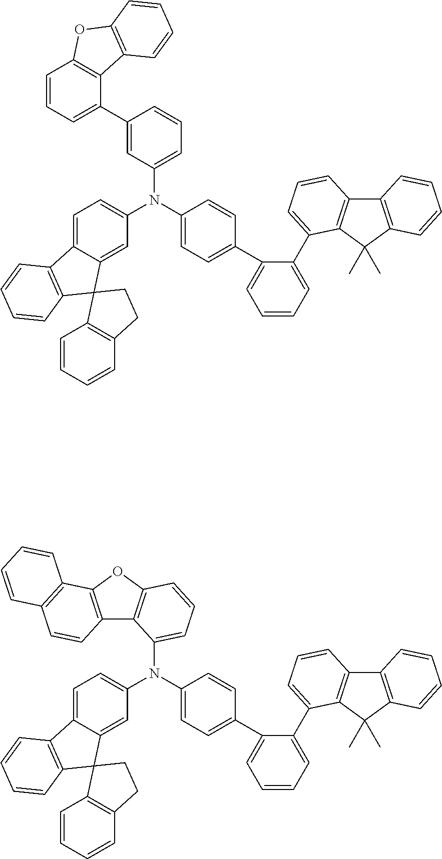 [SMILES: CC1(C)c2ccccc2-c2cccc(-c3ccccc3-c3ccc(N(c4cccc(-c5cccc6oc7ccccc7c56)c4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4cccc5oc6c7ccccc7ccc6c45)cc3)c21]

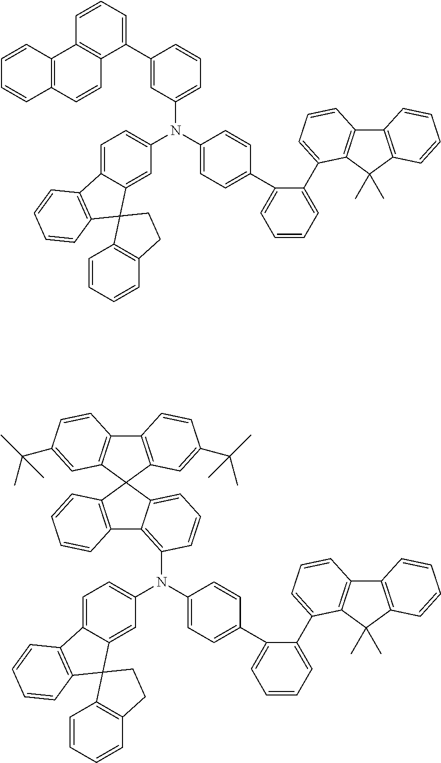 [SMILES: CC1(C)c2ccccc2-c2cccc(-c3ccccc3-c3ccc(N(c4cccc(-c5cccc6c5ccc5ccccc56)c4)c4ccc5c(c4)C4(CCc6ccccc64)c4ccccc4-5)cc3)c21 ; CC(C)(C)c1ccc2c(c1)C1(c3cc(C(C)(C)C)ccc3-2)c2ccccc2-c2c(N(c3ccc(-c4ccccc4-c4cccc5c4C(C)(C)c4ccccc4-5)cc3)c3ccc4c(c3)C3(CCc5ccccc53)c3ccccc3-4)cccc21]

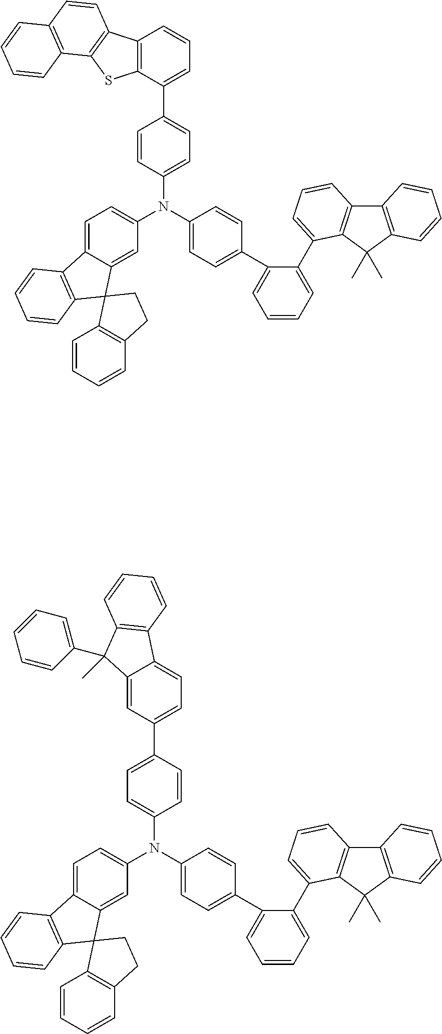 [SMILES: CC1(C)c2ccccc2-c2cccc(-c3ccccc3-c3ccc(N(c4ccc(-c5cccc6c5sc5c7ccccc7ccc65)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(CCc6ccccc64)c4ccccc4-5)cc3)c21]

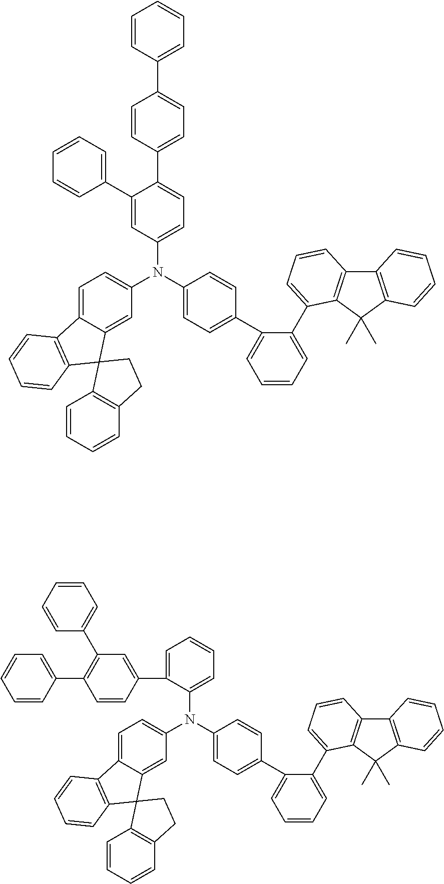 [SMILES: CC1(C)c2ccccc2-c2cccc(-c3ccccc3-c3ccc(N(c4ccc(-c5ccc(-c6ccccc6)cc5)c(-c5ccccc5)c4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4ccc(-c5ccccc5)c(-c5ccccc5)c4)cc3)c21]

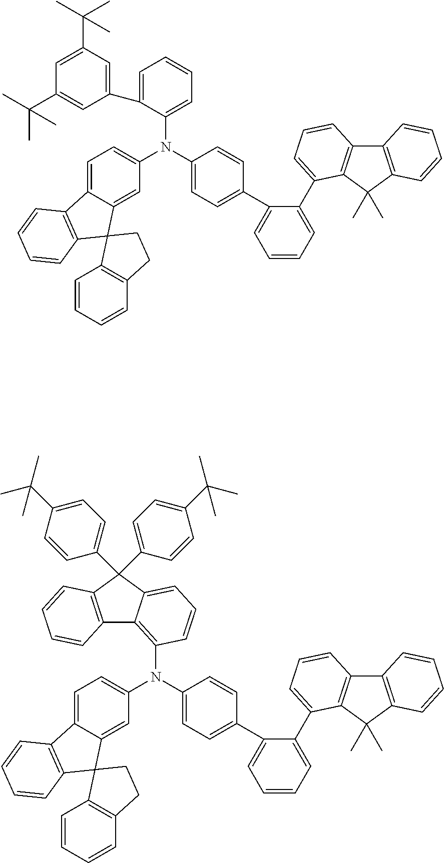 [SMILES: CC(C)(C)c1cc(-c2ccccc2N(c2ccc(-c3ccccc3-c3cccc4c3C(C)(C)c3ccccc3-4)cc2)c2ccc3c(c2)C2(CCc4ccccc42)c2ccccc2-3)cc(C(C)(C)C)c1 ; CC(C)(C)c1ccc(C2(c3ccc(C(C)(C)C)cc3)c3ccccc3-c3c(N(c4ccc(-c5ccccc5-c5cccc6c5C(C)(C)c5ccccc5-6)cc4)c4ccc5c(c4)C4(CCc6ccccc64)c4ccccc4-5)cccc32)cc1]

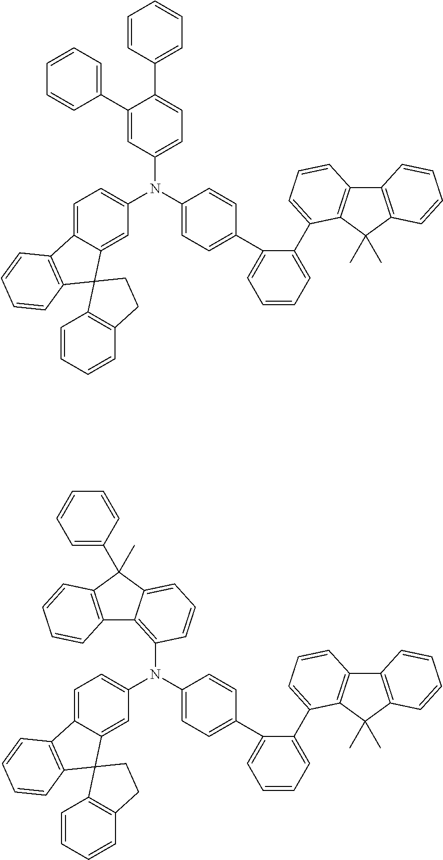 [SMILES: CC1(C)c2ccccc2-c2cccc(-c3ccccc3-c3ccc(N(c4ccc(-c5ccccc5)c(-c5ccccc5)c4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4cccc5c4-c4ccccc4C5(C)c4ccccc4)cc3)c21]

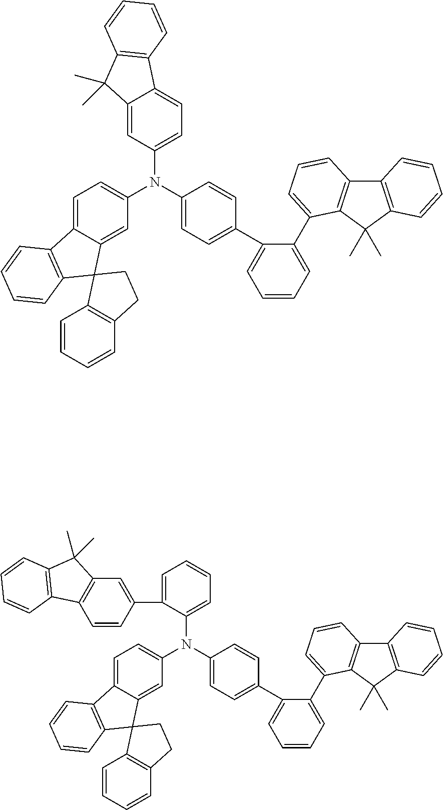 [SMILES: CC1(C)c2ccccc2-c2ccc(N(c3ccc(-c4ccccc4-c4cccc5c4C(C)(C)c4ccccc4-5)cc3)c3ccc4c(c3)C3(CCc5ccccc53)c3ccccc3-4)cc21 ; CC1(C)c2ccccc2-c2ccc(-c3ccccc3N(c3ccc(-c4ccccc4-c4cccc5c4C(C)(C)c4ccccc4-5)cc3)c3ccc4c(c3)C3(CCc5ccccc53)c3ccccc3-4)cc21]

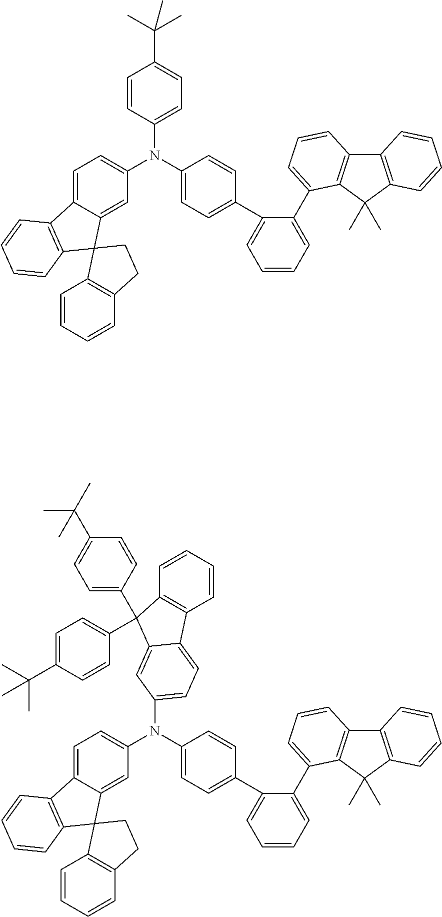 [SMILES: CC(C)(C)c1ccc(N(c2ccc(-c3ccccc3-c3cccc4c3C(C)(C)c3ccccc3-4)cc2)c2ccc3c(c2)C2(CCc4ccccc42)c2ccccc2-3)cc1 ; CC(C)(C)c1ccc(C2(c3ccc(C(C)(C)C)cc3)c3ccccc3-c3ccc(N(c4ccc(-c5ccccc5-c5cccc6c5C(C)(C)c5ccccc5-6)cc4)c4ccc5c(c4)C4(CCc6ccccc64)c4ccccc4-5)cc32)cc1]

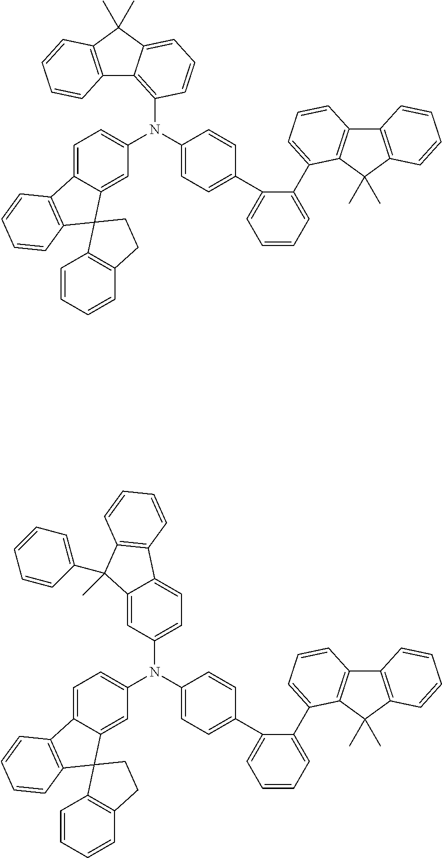 [SMILES: CC1(C)c2ccccc2-c2c(N(c3ccc(-c4ccccc4-c4cccc5c4C(C)(C)c4ccccc4-5)cc3)c3ccc4c(c3)C3(CCc5ccccc53)c3ccccc3-4)cccc21 ; CC1(C)c2ccccc2-c2cccc(-c3ccccc3-c3ccc(N(c4ccc5c(c4)C(C)(c4ccccc4)c4ccccc4-5)c4ccc5c(c4)C4(CCc6ccccc64)c4ccccc4-5)cc3)c21]

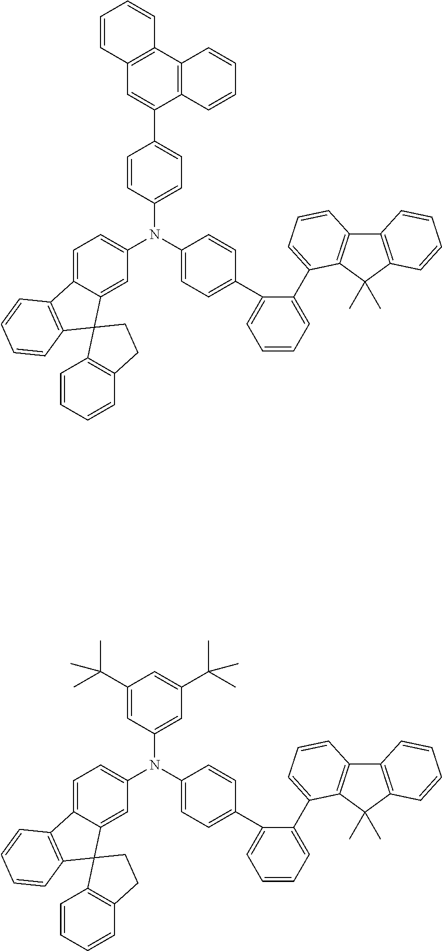 [SMILES: CC1(C)c2ccccc2-c2cccc(-c3ccccc3-c3ccc(N(c4ccc(-c5cc6ccccc6c6ccccc56)cc4)c4ccc5c(c4)C4(CCc6ccccc64)c4ccccc4-5)cc3)c21 ; CC(C)(C)c1cc(N(c2ccc(-c3ccccc3-c3cccc4c3C(C)(C)c3ccccc3-4)cc2)c2ccc3c(c2)C2(CCc4ccccc42)c2ccccc2-3)cc(C(C)(C)C)c1]

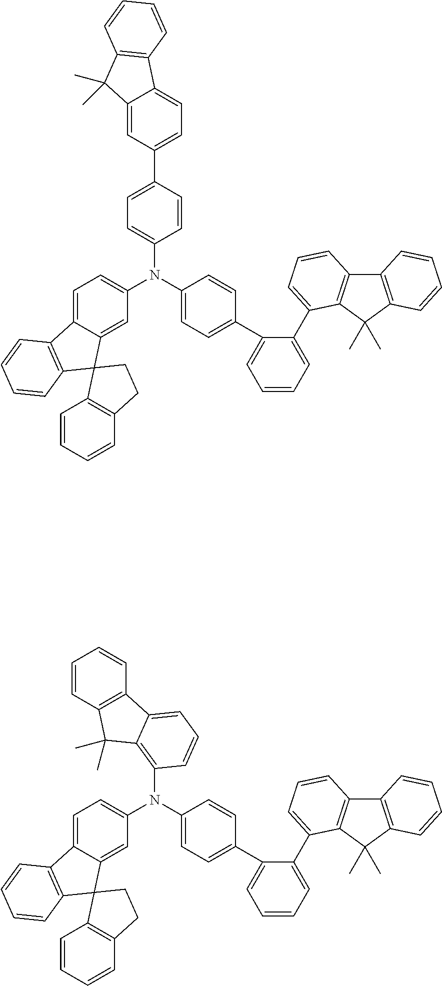 [SMILES: CC1(C)c2ccccc2-c2ccc(-c3ccc(N(c4ccc(-c5ccccc5-c5cccc6c5C(C)(C)c5ccccc5-6)cc4)c4ccc5c(c4)C4(CCc6ccccc64)c4ccccc4-5)cc3)cc21 ; CC1(C)c2ccccc2-c2cccc(-c3ccccc3-c3ccc(N(c4ccc5c(c4)C4(CCc6ccccc64)c4ccccc4-5)c4cccc5c4C(C)(C)c4ccccc4-5)cc3)c21]

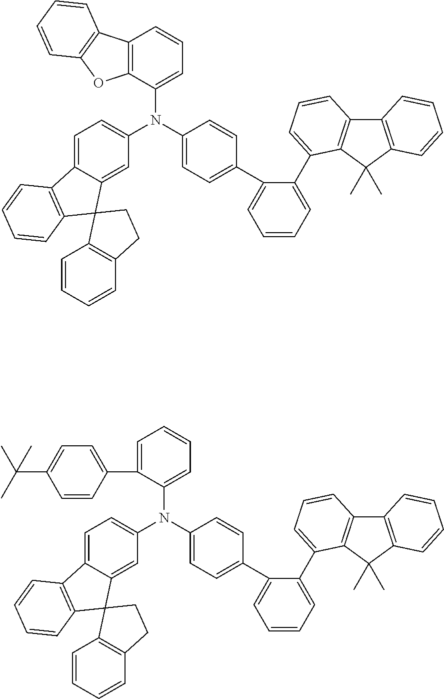 [SMILES: CC1(C)c2ccccc2-c2cccc(-c3ccccc3-c3ccc(N(c4ccc5c(c4)C4(CCc6ccccc64)c4ccccc4-5)c4cccc5c4oc4ccccc45)cc3)c21 ; CC(C)(C)c1ccc(-c2ccccc2N(c2ccc(-c3ccccc3-c3cccc4c3C(C)(C)c3ccccc3-4)cc2)c2ccc3c(c2)C2(CCc4ccccc42)c2ccccc2-3)cc1]

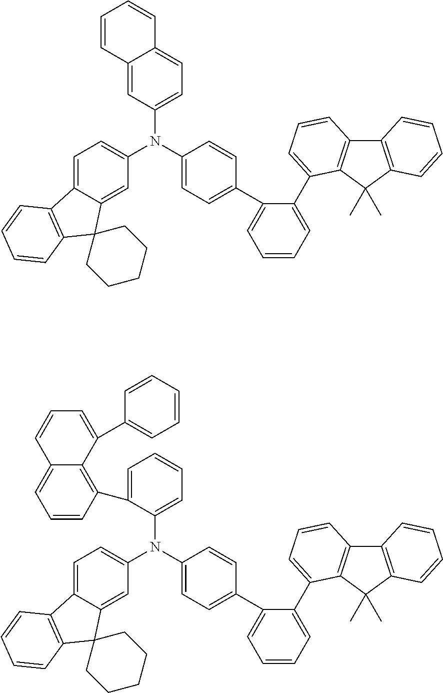 [SMILES: CC1(C)c2ccccc2-c2cccc(-c3ccccc3-c3ccc(N(c4ccc5c(c4)C4(CCCCC4)c4ccccc4-5)c4ccc5ccccc5c4)cc3)c21 ; CC1(C)c2ccccc2-c2cccc(-c3ccccc3-c3ccc(N(c4ccc5c(c4)C4(CCCCC4)c4ccccc4-5)c4ccccc4-c4cccc5cccc(-c6ccccc6)c45)cc3)c21]

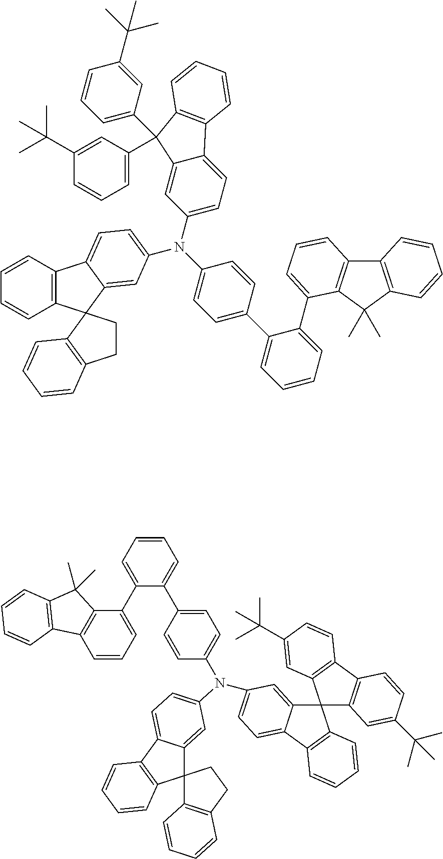 [SMILES: CC(C)(C)c1cccc(C2(c3cccc(C(C)(C)C)c3)c3ccccc3-c3ccc(N(c4ccc(-c5ccccc5-c5cccc6c5C(C)(C)c5ccccc5-6)cc4)c4ccc5c(c4)C4(CCc6ccccc64)c4ccccc4-5)cc32)c1 ; CC(C)(C)c1ccc2c(c1)C1(c3ccccc3-c3ccc(N(c4ccc(-c5ccccc5-c5cccc6c5C(C)(C)c5ccccc5-6)cc4)c4ccc5c(c4)C4(CCc6ccccc64)c4ccccc4-5)cc31)c1cc(C(C)(C)C)ccc1-2]

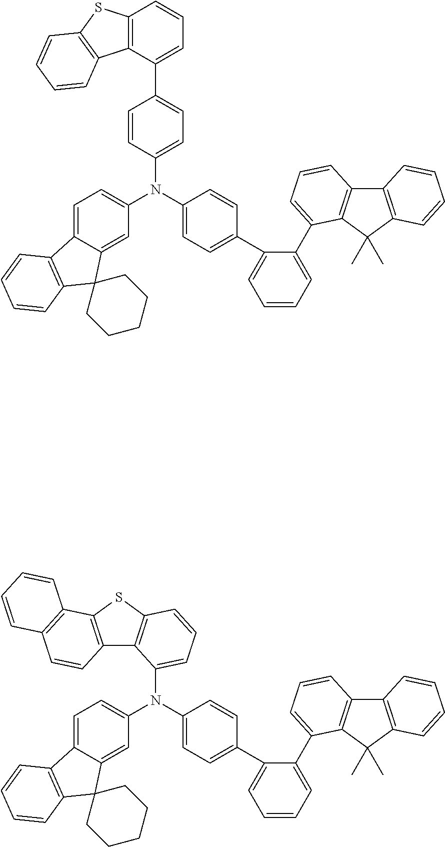 [SMILES: CC1(C)c2ccccc2-c2cccc(-c3ccccc3-c3ccc(N(c4ccc(-c5cccc6sc7ccccc7c56)cc4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4cccc5sc6c7ccccc7ccc6c45)cc3)c21]

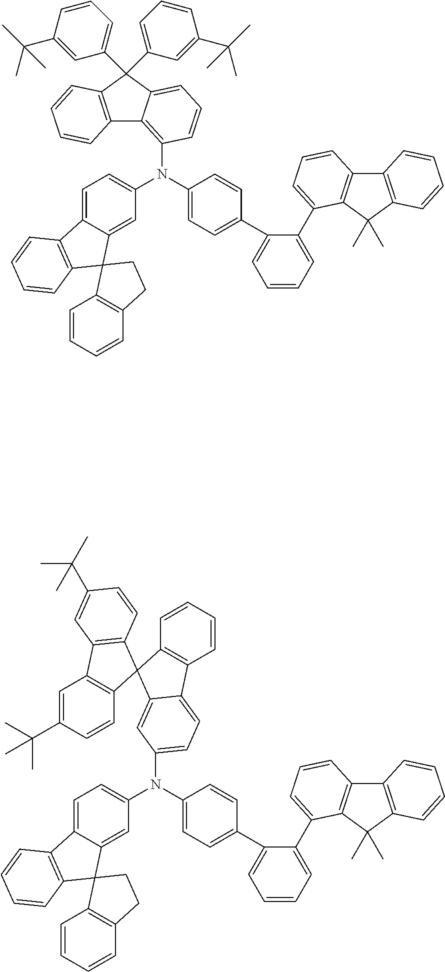 [SMILES: CC(C)(C)c1cccc(C2(c3cccc(C(C)(C)C)c3)c3ccccc3-c3c(N(c4ccc(-c5ccccc5-c5cccc6c5C(C)(C)c5ccccc5-6)cc4)c4ccc5c(c4)C4(CCc6ccccc64)c4ccccc4-5)cccc32)c1 ; CC(C)(C)c1ccc2c(c1)-c1cc(C(C)(C)C)ccc1C21c2ccccc2-c2ccc(N(c3ccc(-c4ccccc4-c4cccc5c4C(C)(C)c4ccccc4-5)cc3)c3ccc4c(c3)C3(CCc5ccccc53)c3ccccc3-4)cc21]

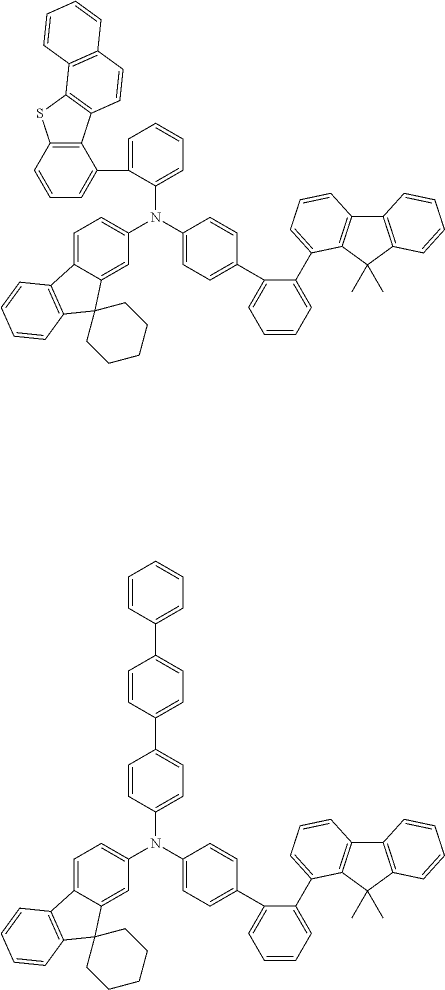 [SMILES: CC1(C)c2ccccc2-c2cccc(-c3ccccc3-c3ccc(N(c4ccc5c(c4)C4(CCCCC4)c4ccccc4-5)c4ccccc4-c4cccc5sc6c7ccccc7ccc6c45)cc3)c21 ; CC1(C)c2ccccc2-c2cccc(-c3ccccc3-c3ccc(N(c4ccc(-c5ccc(-c6ccccc6)cc5)cc4)c4ccc5c(c4)C4(CCCCC4)c4ccccc4-5)cc3)c21]

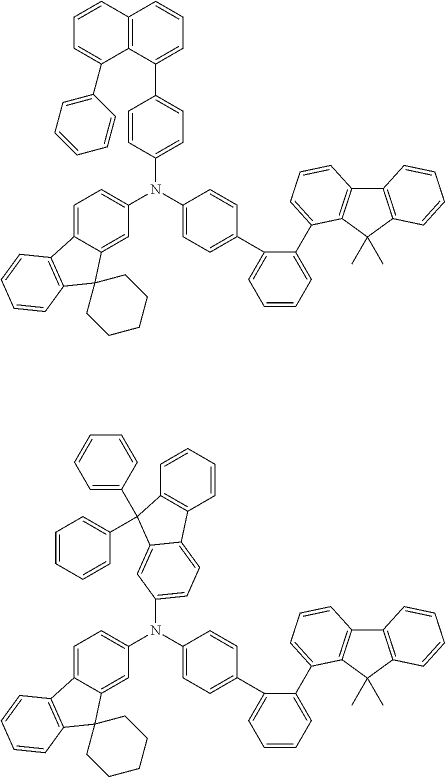 [SMILES: CC1(C)c2ccccc2-c2cccc(-c3ccccc3-c3ccc(N(c4ccc(-c5cccc6cccc(-c7ccccc7)c56)cc4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4ccc5c(c4)C(c4ccccc4)(c4ccccc4)c4ccccc4-5)cc3)c21]

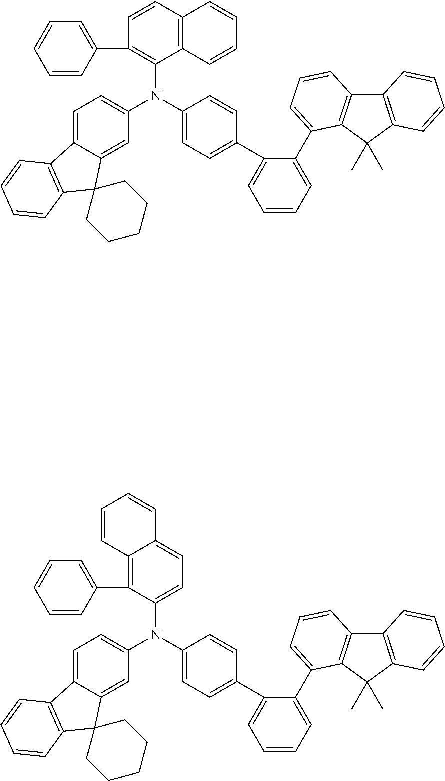 [SMILES: CC1(C)c2ccccc2-c2cccc(-c3ccccc3-c3ccc(N(c4ccc5c(c4)C4(CCCCC4)c4ccccc4-5)c4c(-c5ccccc5)ccc5ccccc45)cc3)c21 ; CC1(C)c2ccccc2-c2cccc(-c3ccccc3-c3ccc(N(c4ccc5c(c4)C4(CCCCC4)c4ccccc4-5)c4ccc5ccccc5c4-c4ccccc4)cc3)c21]

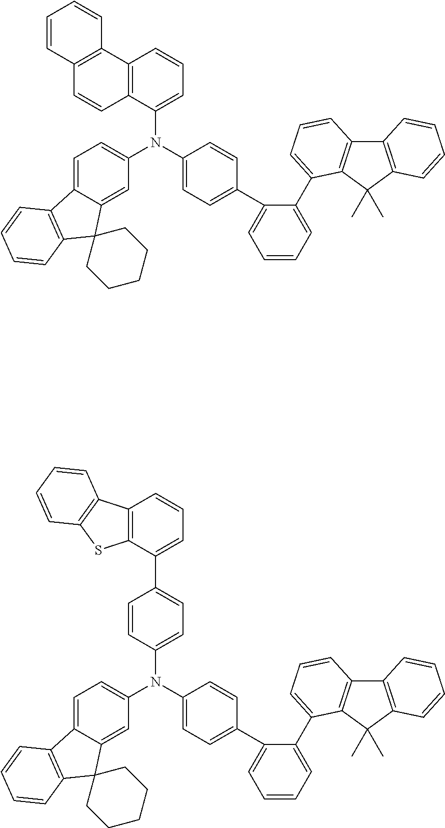 [SMILES: CC1(C)c2ccccc2-c2cccc(-c3ccccc3-c3ccc(N(c4ccc5c(c4)C4(CCCCC4)c4ccccc4-5)c4cccc5c4ccc4ccccc45)cc3)c21 ; CC1(C)c2ccccc2-c2cccc(-c3ccccc3-c3ccc(N(c4ccc(-c5cccc6c5sc5ccccc56)cc4)c4ccc5c(c4)C4(CCCCC4)c4ccccc4-5)cc3)c21]

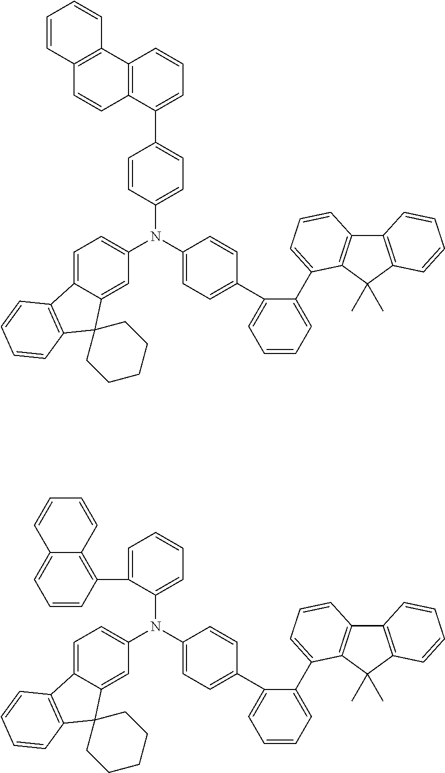 [SMILES: CC1(C)c2ccccc2-c2cccc(-c3ccccc3-c3ccc(N(c4ccc(-c5cccc6c5ccc5ccccc56)cc4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4ccccc4-c4cccc5ccccc45)cc3)c21]

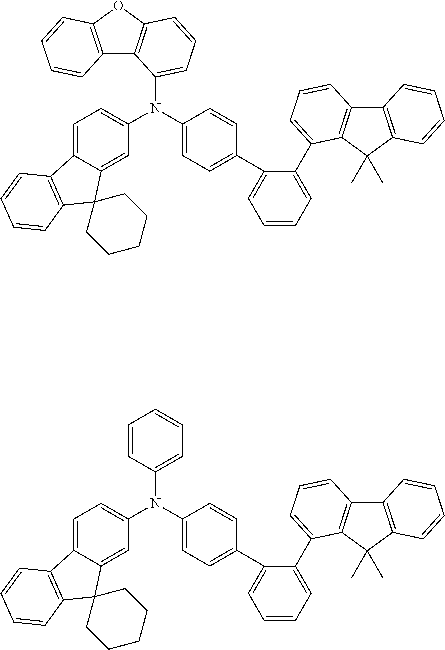 [SMILES: CC1(C)c2ccccc2-c2cccc(-c3ccccc3-c3ccc(N(c4ccc5c(c4)C4(CCCCC4)c4ccccc4-5)c4cccc5oc6ccccc6c45)cc3)c21 ; CC1(C)c2ccccc2-c2cccc(-c3ccccc3-c3ccc(N(c4ccccc4)c4ccc5c(c4)C4(CCCCC4)c4ccccc4-5)cc3)c21]

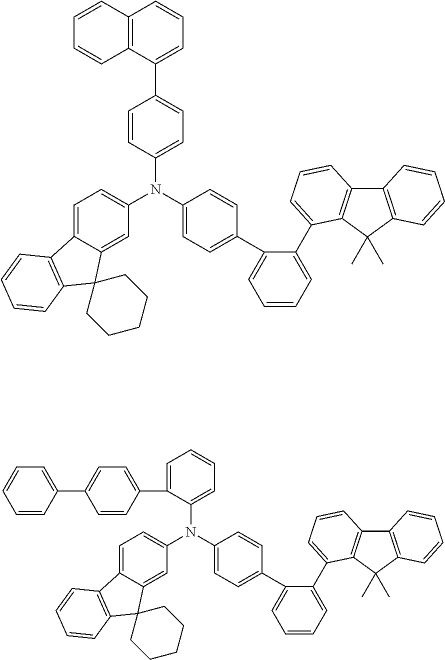 [SMILES: CC1(C)c2ccccc2-c2cccc(-c3ccccc3-c3ccc(N(c4ccc(-c5cccc6ccccc56)cc4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4ccccc4-c4ccc(-c5ccccc5)cc4)cc3)c21]

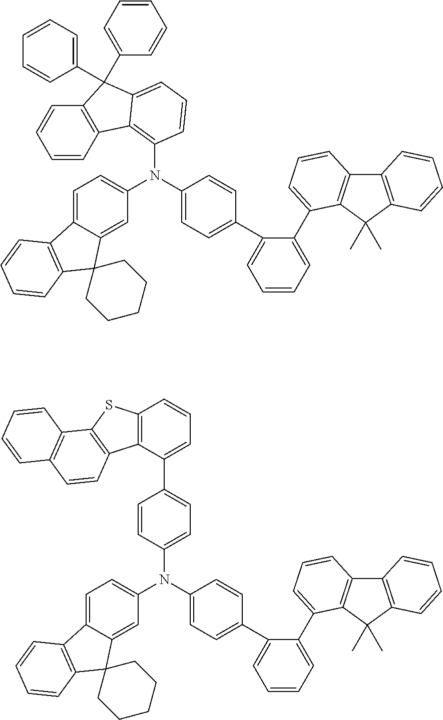 [SMILES: CC1(C)c2ccccc2-c2cccc(-c3ccccc3-c3ccc(N(c4ccc5c(c4)C4(CCCCC4)c4ccccc4-5)c4cccc5c4-c4ccccc4C5(c4ccccc4)c4ccccc4)cc3)c21 ; CC1(C)c2ccccc2-c2cccc(-c3ccccc3-c3ccc(N(c4ccc(-c5cccc6sc7c8ccccc8ccc7c56)cc4)c4ccc5c(c4)C4(CCCCC4)c4ccccc4-5)cc3)c21]

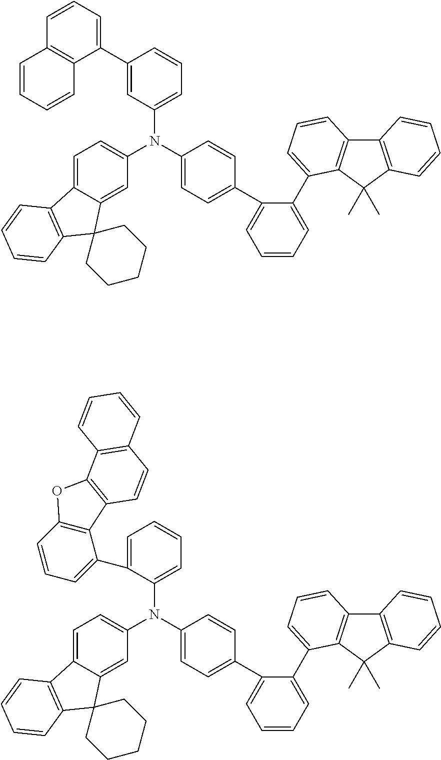 [SMILES: CC1(C)c2ccccc2-c2cccc(-c3ccccc3-c3ccc(N(c4cccc(-c5cccc6ccccc56)c4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4ccccc4-c4cccc5oc6c7ccccc7ccc6c45)cc3)c21]

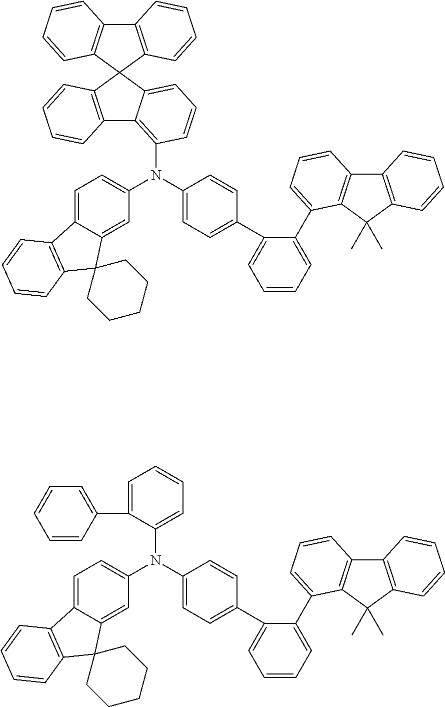 [SMILES: CC1(C)c2ccccc2-c2cccc(-c3ccccc3-c3ccc(N(c4ccc5c(c4)C4(CCCCC4)c4ccccc4-5)c4cccc5c4-c4ccccc4C54c5ccccc5-c5ccccc54)cc3)c21 ; CC1(C)c2ccccc2-c2cccc(-c3ccccc3-c3ccc(N(c4ccc5c(c4)C4(CCCCC4)c4ccccc4-5)c4ccccc4-c4ccccc4)cc3)c21]

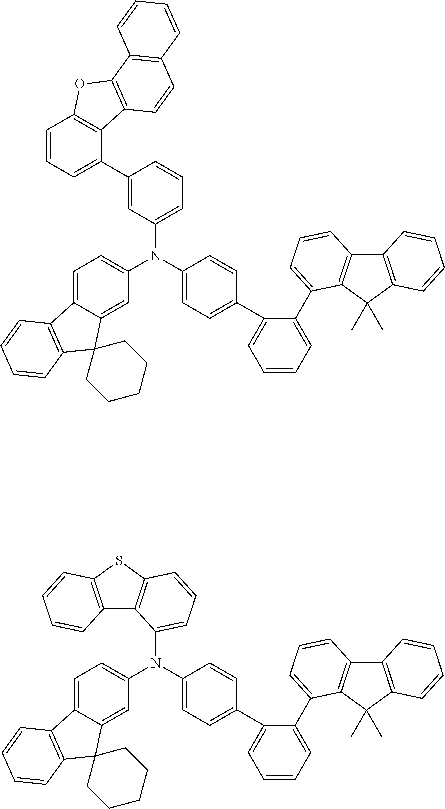 [SMILES: CC1(C)c2ccccc2-c2cccc(-c3ccccc3-c3ccc(N(c4cccc(-c5cccc6oc7c8ccccc8ccc7c56)c4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4cccc5sc6ccccc6c45)cc3)c21]

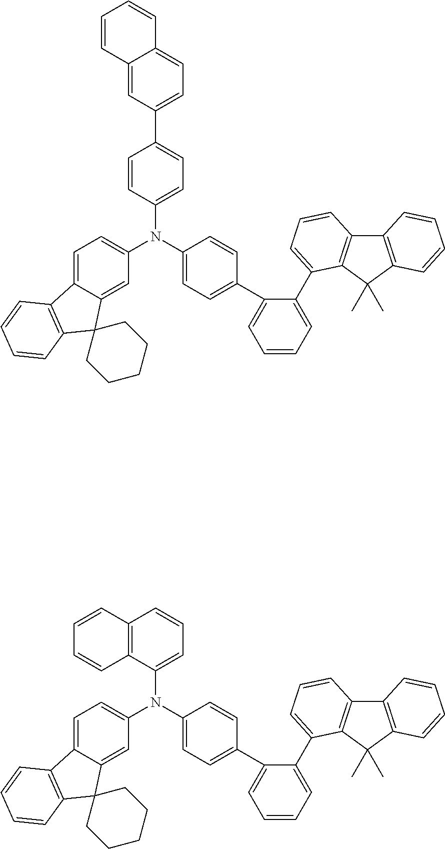 [SMILES: CC1(C)c2ccccc2-c2cccc(-c3ccccc3-c3ccc(N(c4ccc(-c5ccc6ccccc6c5)cc4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4cccc5ccccc45)cc3)c21]

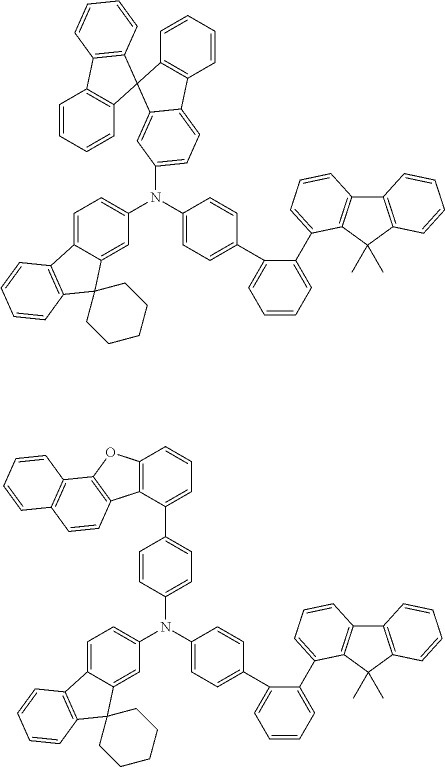 [SMILES: CC1(C)c2ccccc2-c2cccc(-c3ccccc3-c3ccc(N(c4ccc5c(c4)C4(CCCCC4)c4ccccc4-5)c4ccc5c(c4)C4(c6ccccc6-c6ccccc64)c4ccccc4-5)cc3)c21 ; CC1(C)c2ccccc2-c2cccc(-c3ccccc3-c3ccc(N(c4ccc(-c5cccc6oc7c8ccccc8ccc7c56)cc4)c4ccc5c(c4)C4(CCCCC4)c4ccccc4-5)cc3)c21]

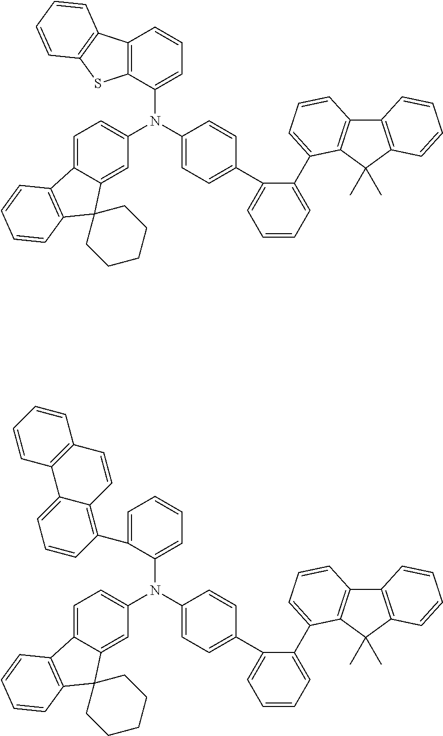 [SMILES: CC1(C)c2ccccc2-c2cccc(-c3ccccc3-c3ccc(N(c4ccc5c(c4)C4(CCCCC4)c4ccccc4-5)c4cccc5c4sc4ccccc45)cc3)c21 ; CC1(C)c2ccccc2-c2cccc(-c3ccccc3-c3ccc(N(c4ccc5c(c4)C4(CCCCC4)c4ccccc4-5)c4ccccc4-c4cccc5c4ccc4ccccc45)cc3)c21]

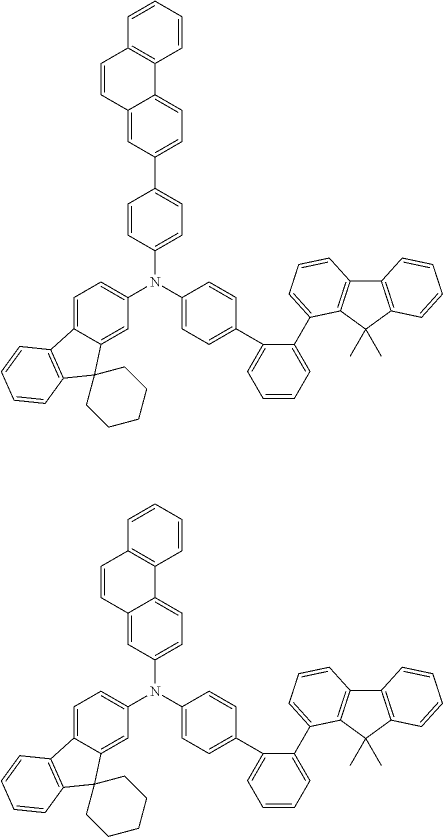 [SMILES: CC1(C)c2ccccc2-c2cccc(-c3ccccc3-c3ccc(N(c4ccc(-c5ccc6c(ccc7ccccc76)c5)cc4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4ccc5c(ccc6ccccc65)c4)cc3)c21]

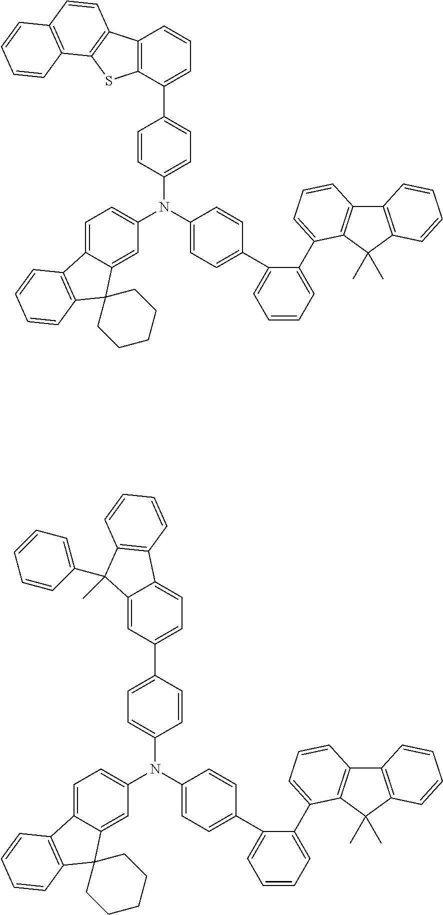 [SMILES: CC1(C)c2ccccc2-c2cccc(-c3ccccc3-c3ccc(N(c4ccc(-c5cccc6c5sc5c7ccccc7ccc65)cc4)c4ccc5c(c4)C4(CCCCC4)c4ccccc4-5)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(CCCCC4)c4ccccc4-5)cc3)c21]

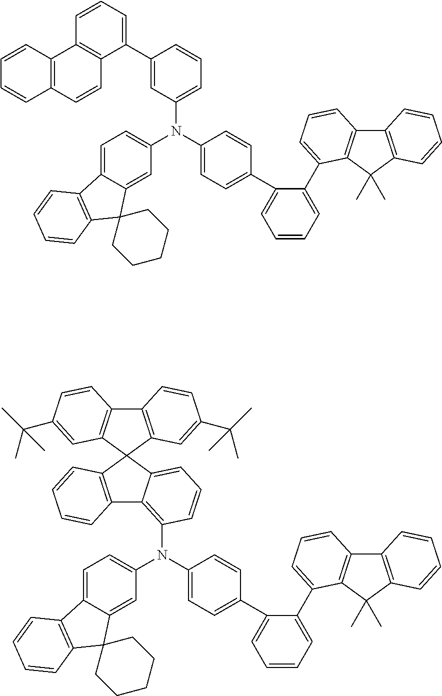 [SMILES: CC1(C)c2ccccc2-c2cccc(-c3ccccc3-c3ccc(N(c4cccc(-c5cccc6c5ccc5ccccc56)c4)c4ccc5c(c4)C4(CCCCC4)c4ccccc4-5)cc3)c21 ; CC(C)(C)c1ccc2c(c1)C1(c3cc(C(C)(C)C)ccc3-2)c2ccccc2-c2c(N(c3ccc(-c4ccccc4-c4cccc5c4C(C)(C)c4ccccc4-5)cc3)c3ccc4c(c3)C3(CCCCC3)c3ccccc3-4)cccc21]

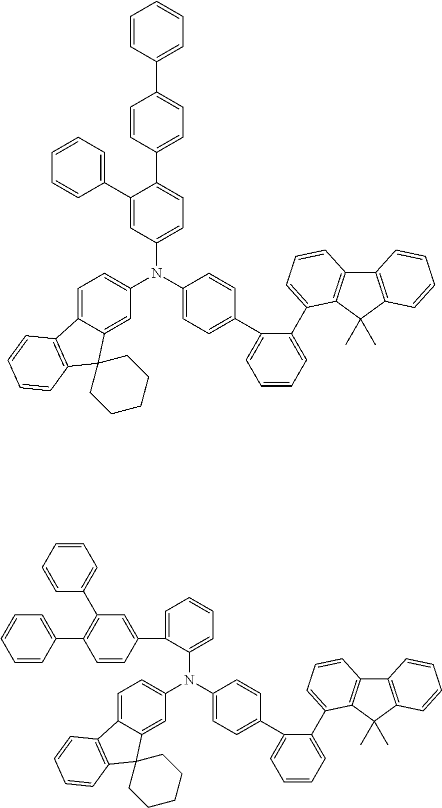 [SMILES: CC1(C)c2ccccc2-c2cccc(-c3ccccc3-c3ccc(N(c4ccc(-c5ccc(-c6ccccc6)cc5)c(-c5ccccc5)c4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4ccccc4-c4ccc(-c5ccccc5)c(-c5ccccc5)c4)cc3)c21]

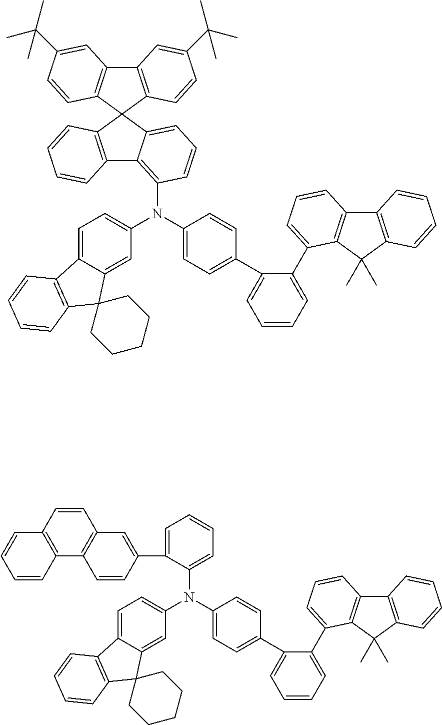 [SMILES: CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C3(CCCCC3)c3ccccc3-4)cccc21 ; CC1(C)c2ccccc2-c2cccc(-c3ccccc3-c3ccc(N(c4ccc5c(c4)C4(CCCCC4)c4ccccc4-5)c4ccccc4-c4ccc5c(ccc6ccccc65)c4)cc3)c21]

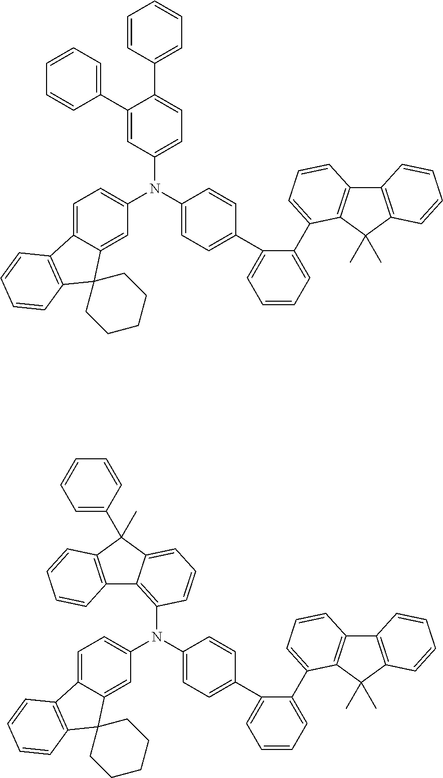 [SMILES: CC1(C)c2ccccc2-c2cccc(-c3ccccc3-c3ccc(N(c4ccc(-c5ccccc5)c(-c5ccccc5)c4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4cccc5c4-c4ccccc4C5(C)c4ccccc4)cc3)c21]

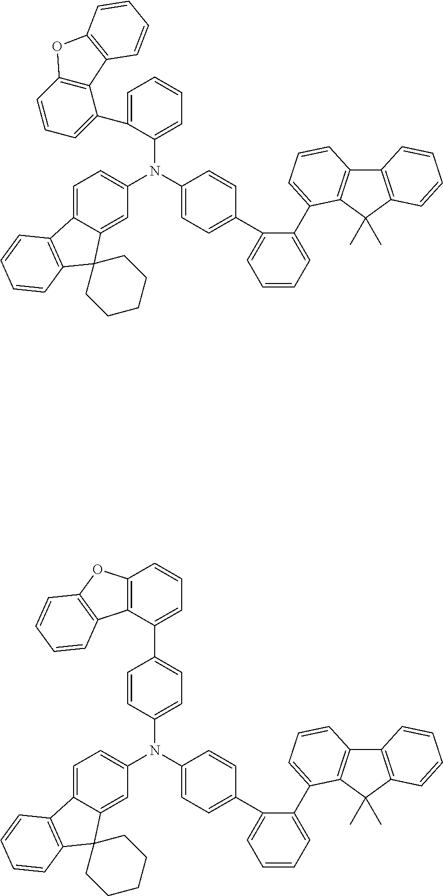 [SMILES: CC1(C)c2ccccc2-c2cccc(-c3ccccc3-c3ccc(N(c4ccc5c(c4)C4(CCCCC4)c4ccccc4-5)c4ccccc4-c4cccc5oc6ccccc6c45)cc3)c21 ; CC1(C)c2ccccc2-c2cccc(-c3ccccc3-c3ccc(N(c4ccc(-c5cccc6oc7ccccc7c56)cc4)c4ccc5c(c4)C4(CCCCC4)c4ccccc4-5)cc3)c21]

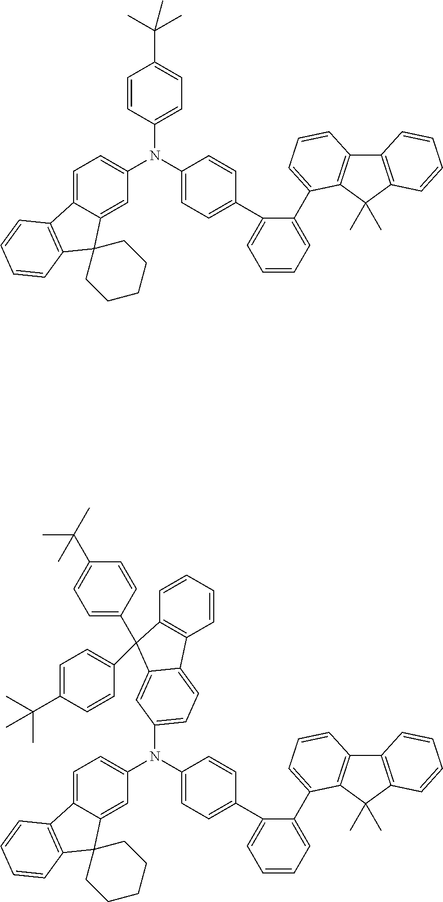 [SMILES: CC(C)(C)c1ccc(N(c2ccc(-c3ccccc3-c3cccc4c3C(C)(C)c3ccccc3-4)cc2)c2ccc3c(c2)C2(CCCCC2)c2ccccc2-3)cc1 ; CC(C)(C)c1ccc(C2(c3ccc(C(C)(C)C)cc3)c3ccccc3-c3ccc(N(c4ccc(-c5ccccc5-c5cccc6c5C(C)(C)c5ccccc5-6)cc4)c4ccc5c(c4)C4(CCCCC4)c4ccccc4-5)cc32)cc1]

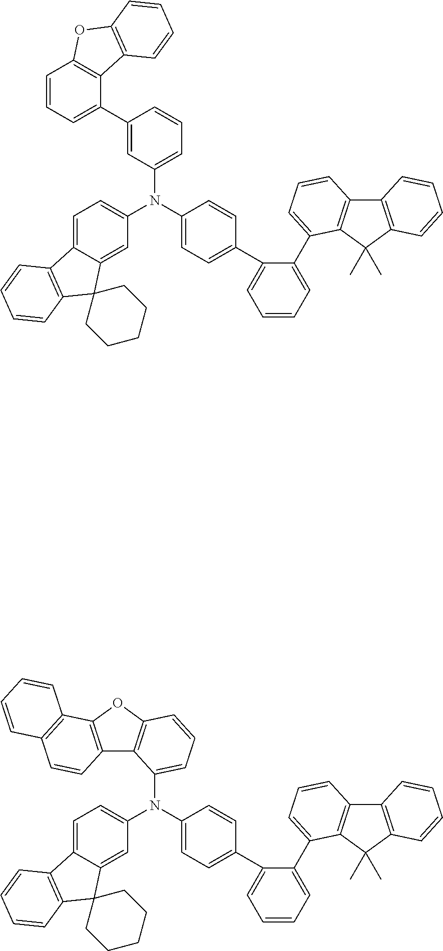 [SMILES: CC1(C)c2ccccc2-c2cccc(-c3ccccc3-c3ccc(N(c4cccc(-c5cccc6oc7ccccc7c56)c4)c4ccc5c(c4)C4(CCCCC4)c4ccccc4-5)cc3)c21 ; CC1(C)c2ccccc2-c2cccc(-c3ccccc3-c3ccc(N(c4ccc5c(c4)C4(CCCCC4)c4ccccc4-5)c4cccc5oc6c7ccccc7ccc6c45)cc3)c21]

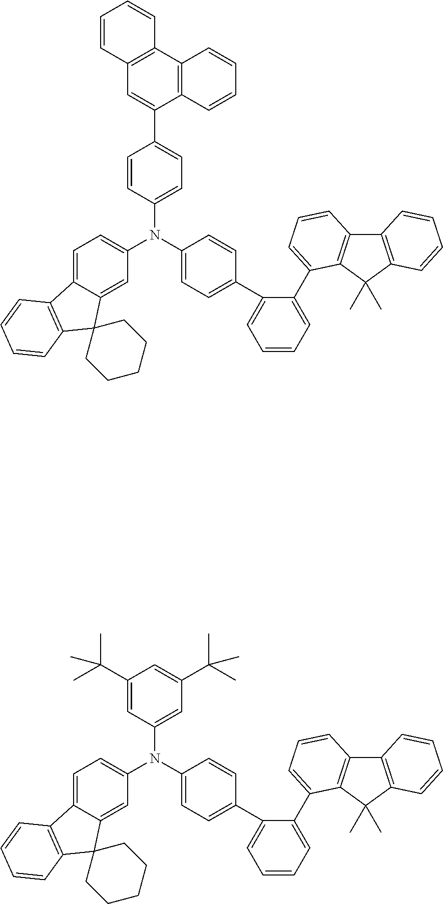 [SMILES: CC1(C)c2ccccc2-c2cccc(-c3ccccc3-c3ccc(N(c4ccc(-c5cc6ccccc6c6ccccc56)cc4)c4ccc5c(c4)C4(CCCCC4)c4ccccc4-5)cc3)c21 ; CC(C)(C)c1cc(N(c2ccc(-c3ccccc3-c3cccc4c3C(C)(C)c3ccccc3-4)cc2)c2ccc3c(c2)C2(CCCCC2)c2ccccc2-3)cc(C(C)(C)C)c1]

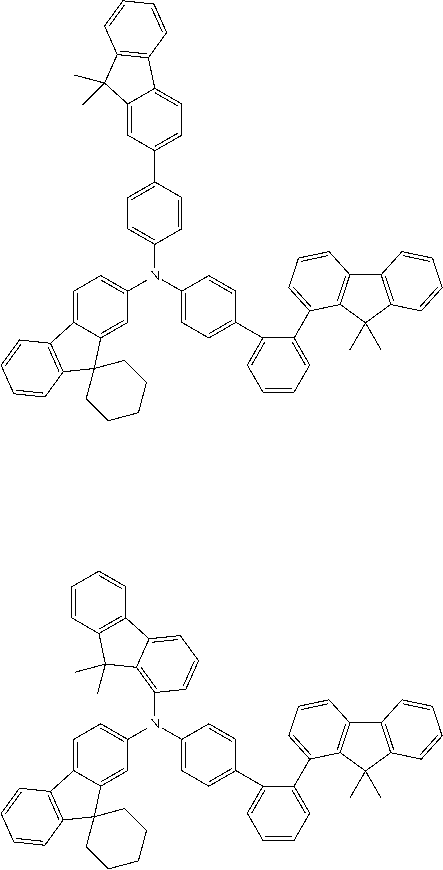 [SMILES: CC1(C)c2ccccc2-c2ccc(-c3ccc(N(c4ccc(-c5ccccc5-c5cccc6c5C(C)(C)c5ccccc5-6)cc4)c4ccc5c(c4)C4(CCCCC4)c4ccccc4-5)cc3)cc21 ; CC1(C)c2ccccc2-c2cccc(-c3ccccc3-c3ccc(N(c4ccc5c(c4)C4(CCCCC4)c4ccccc4-5)c4cccc5c4C(C)(C)c4ccccc4-5)cc3)c21]

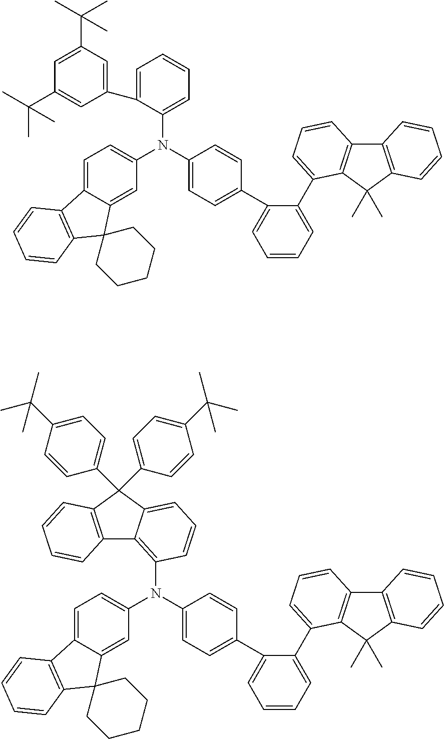 [SMILES: CC(C)(C)c1cc(-c2ccccc2N(c2ccc(-c3ccccc3-c3cccc4c3C(C)(C)c3ccccc3-4)cc2)c2ccc3c(c2)C2(CCCCC2)c2ccccc2-3)cc(C(C)(C)C)c1 ; CC(C)(C)c1ccc(C2(c3ccc(C(C)(C)C)cc3)c3ccccc3-c3c(N(c4ccc(-c5ccccc5-c5cccc6c5C(C)(C)c5ccccc5-6)cc4)c4ccc5c(c4)C4(CCCCC4)c4ccccc4-5)cccc32)cc1]

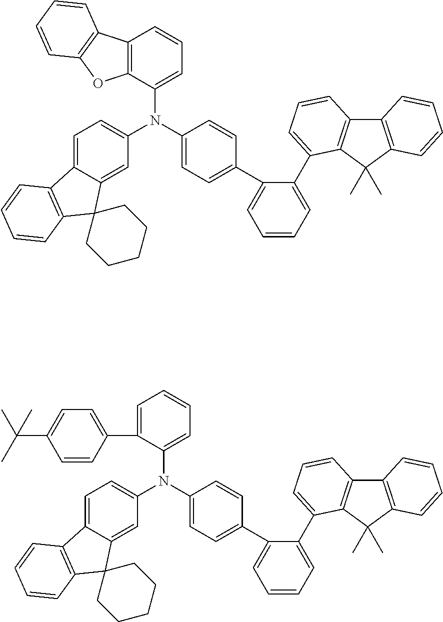 [SMILES: CC1(C)c2ccccc2-c2cccc(-c3ccccc3-c3ccc(N(c4ccc5c(c4)C4(CCCCC4)c4ccccc4-5)c4cccc5c4oc4ccccc45)cc3)c21 ; CC(C)(C)c1ccc(-c2ccccc2N(c2ccc(-c3ccccc3-c3cccc4c3C(C)(C)c3ccccc3-4)cc2)c2ccc3c(c2)C2(CCCCC2)c2ccccc2-3)cc1]

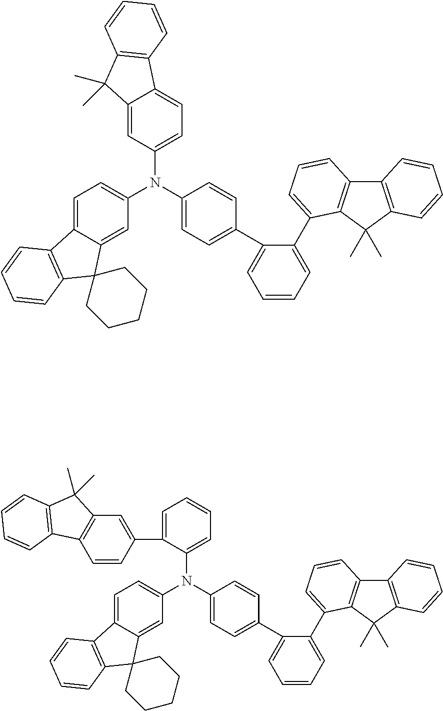 [SMILES: CC1(C)c2ccccc2-c2ccc(N(c3ccc(-c4ccccc4-c4cccc5c4C(C)(C)c4ccccc4-5)cc3)c3ccc4c(c3)C3(CCCCC3)c3ccccc3-4)cc21 ; CC1(C)c2ccccc2-c2ccc(-c3ccccc3N(c3ccc(-c4ccccc4-c4cccc5c4C(C)(C)c4ccccc4-5)cc3)c3ccc4c(c3)C3(CCCCC3)c3ccccc3-4)cc21]

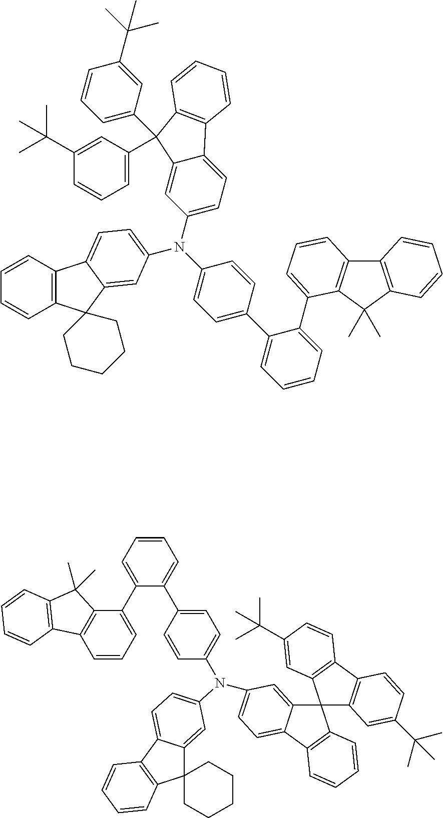 [SMILES: CC(C)(C)c1cccc(C2(c3cccc(C(C)(C)C)c3)c3ccccc3-c3ccc(N(c4ccc(-c5ccccc5-c5cccc6c5C(C)(C)c5ccccc5-6)cc4)c4ccc5c(c4)C4(CCCCC4)c4ccccc4-5)cc32)c1 ; CC(C)(C)c1ccc2c(c1)C1(c3ccccc3-c3ccc(N(c4ccc(-c5ccccc5-c5cccc6c5C(C)(C)c5ccccc5-6)cc4)c4ccc5c(c4)C4(CCCCC4)c4ccccc4-5)cc31)c1cc(C(C)(C)C)ccc1-2]

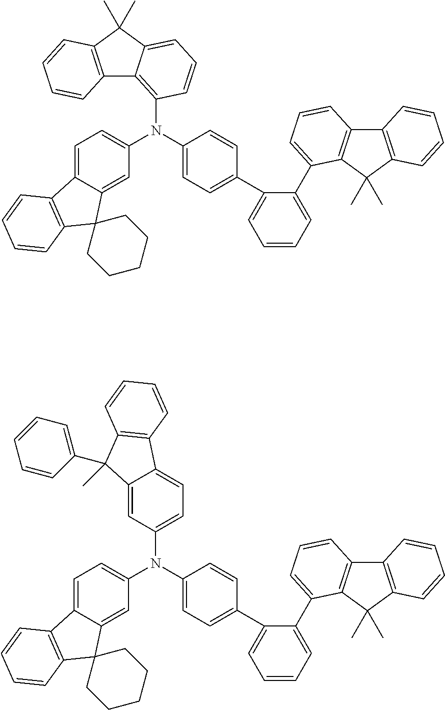 [SMILES: CC1(C)c2ccccc2-c2c(N(c3ccc(-c4ccccc4-c4cccc5c4C(C)(C)c4ccccc4-5)cc3)c3ccc4c(c3)C3(CCCCC3)c3ccccc3-4)cccc21 ; CC1(C)c2ccccc2-c2cccc(-c3ccccc3-c3ccc(N(c4ccc5c(c4)C4(CCCCC4)c4ccccc4-5)c4ccc5c(c4)C(C)(c4ccccc4)c4ccccc4-5)cc3)c21]

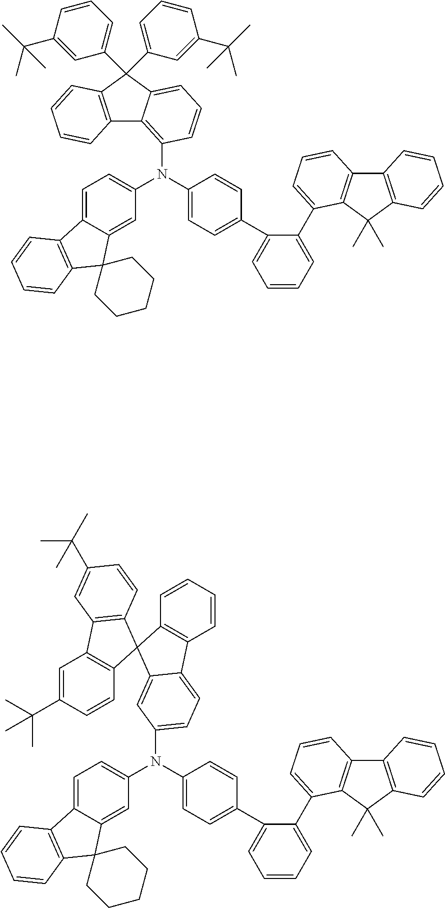 [SMILES: CC(C)(C)c1cccc(C2(c3cccc(C(C)(C)C)c3)c3ccccc3-c3c(N(c4ccc(-c5ccccc5-c5cccc6c5C(C)(C)c5ccccc5-6)cc4)c4ccc5c(c4)C4(CCCCC4)c4ccccc4-5)cccc32)c1 ; CC(C)(C)c1ccc2c(c1)-c1cc(C(C)(C)C)ccc1C21c2ccccc2-c2ccc(N(c3ccc(-c4ccccc4-c4cccc5c4C(C)(C)c4ccccc4-5)cc3)c3ccc4c(c3)C3(CCCCC3)c3ccccc3-4)cc21]

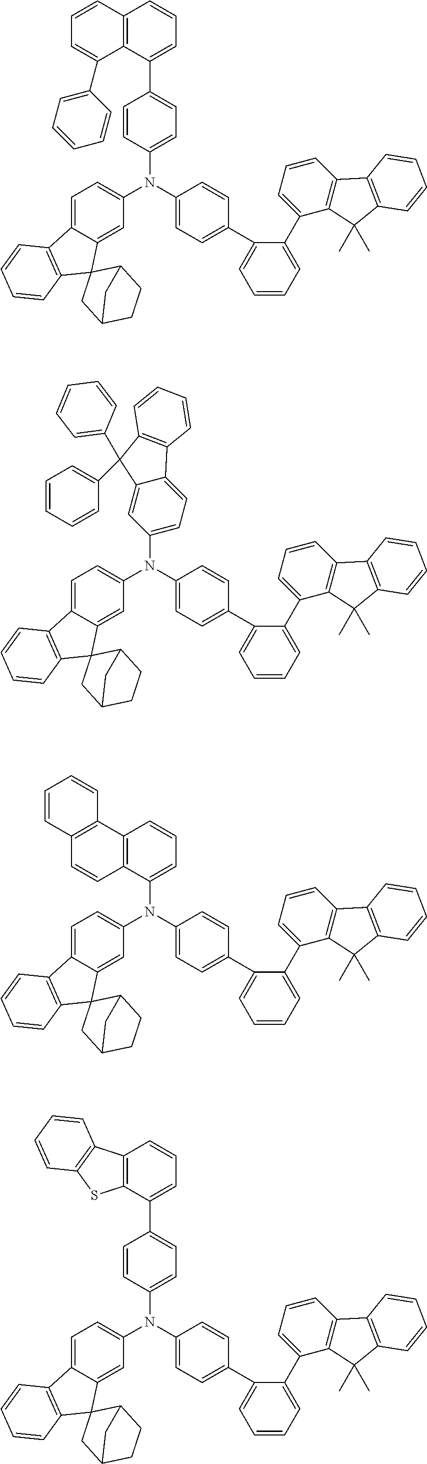 [SMILES: CC1(C)c2ccccc2-c2cccc(-c3ccccc3-c3ccc(N(c4ccc(-c5cccc6cccc(-c7ccccc7)c56)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C(c4ccccc4)(c4ccccc4)c4ccccc4-5)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4cccc5c4ccc4ccccc45)cc3)c21 ; CC1(C)c2ccccc2-c2cccc(-c3ccccc3-c3ccc(N(c4ccc(-c5cccc6c5sc5ccccc56)cc4)c4ccc5c(c4)C4(CC6CCC4C6)c4ccccc4-5)cc3)c21]

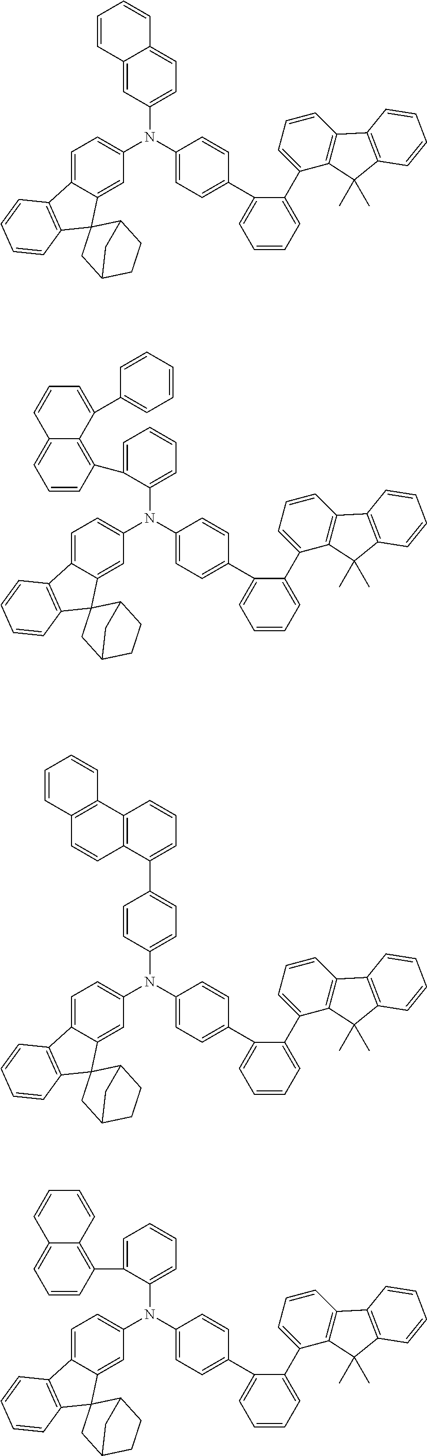 [SMILES: CC1(C)c2ccccc2-c2cccc(-c3ccccc3-c3ccc(N(c4ccc5c(c4)C4(CC6CCC4C6)c4ccccc4-5)c4ccc5ccccc5c4)cc3)c21 ; CC1(C)c2ccccc2-c2cccc(-c3ccccc3-c3ccc(N(c4ccc5c(c4)C4(CC6CCC4C6)c4ccccc4-5)c4ccccc4-c4cccc5cccc(-c6ccccc6)c45)cc3)c21 ; CC1(C)c2ccccc2-c2cccc(-c3ccccc3-c3ccc(N(c4ccc(-c5cccc6c5ccc5ccccc56)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4ccccc4-c4cccc5ccccc45)cc3)c21]

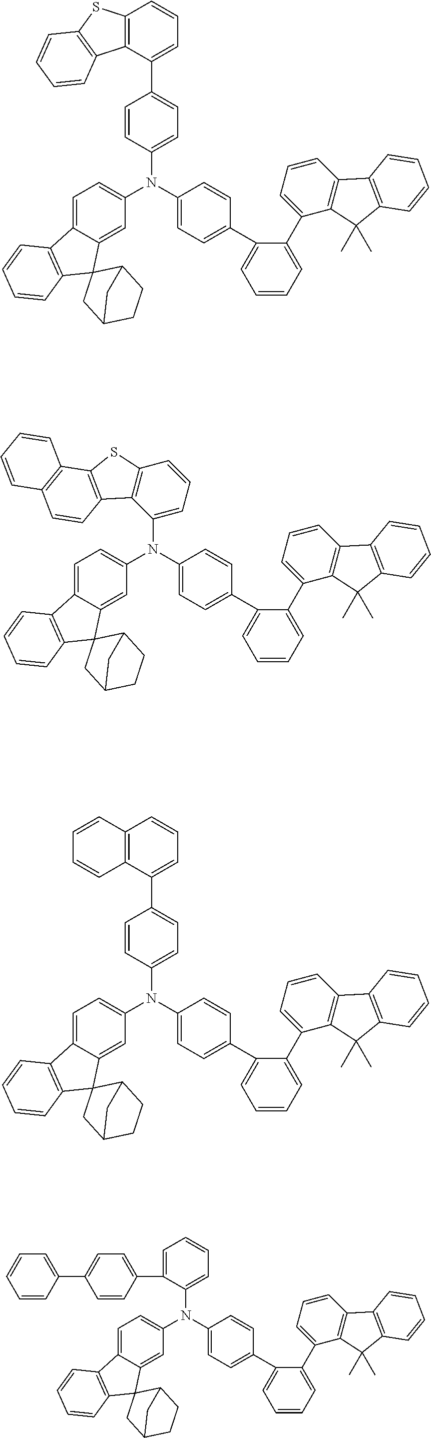 [SMILES: CC1(C)c2ccccc2-c2cccc(-c3ccccc3-c3ccc(N(c4ccc(-c5cccc6sc7ccccc7c56)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4cccc5sc6c7ccccc7ccc6c45)cc3)c21 ; CC1(C)c2ccccc2-c2cccc(-c3ccccc3-c3ccc(N(c4ccc(-c5cccc6ccccc56)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4ccccc4-c4ccc(-c5ccccc5)cc4)cc3)c21]

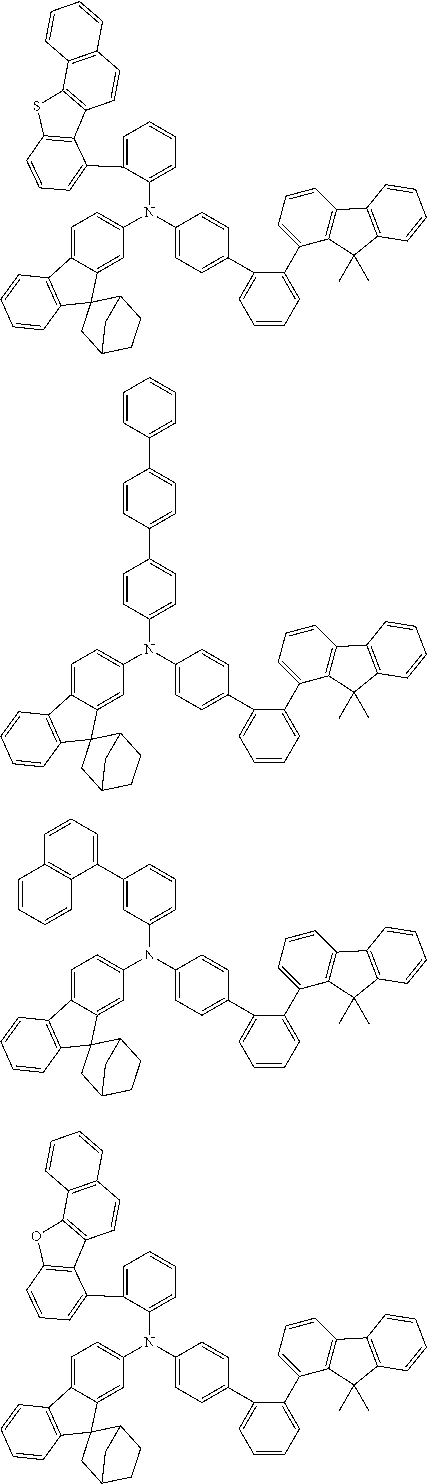

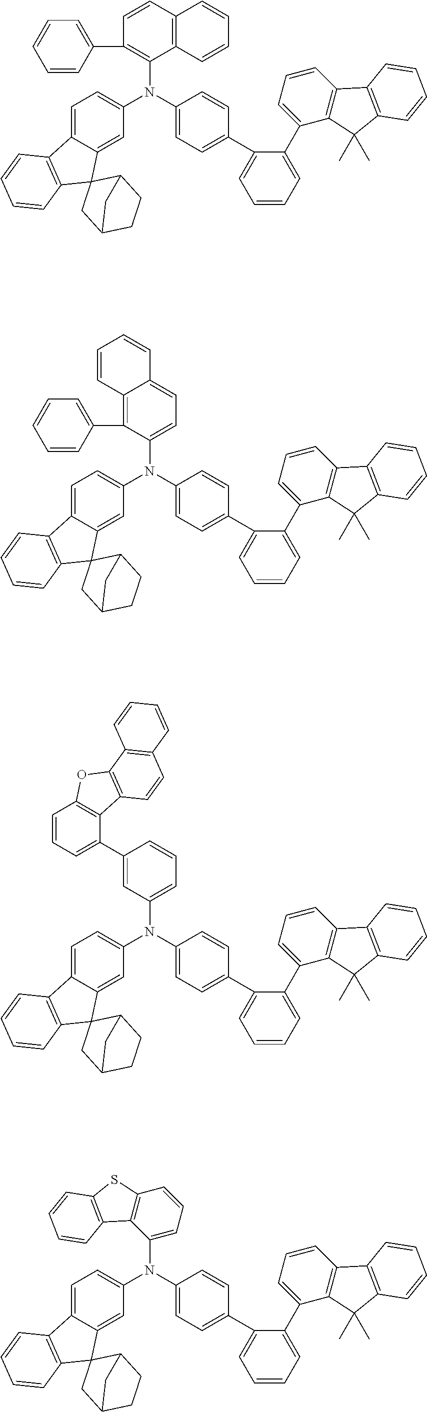 [SMILES: CC1(C)c2ccccc2-c2cccc(-c3ccccc3-c3ccc(N(c4ccc5c(c4)C4(CC6CCC4C6)c4ccccc4-5)c4c(-c5ccccc5)ccc5ccccc45)cc3)c21 ; CC1(C)c2ccccc2-c2cccc(-c3ccccc3-c3ccc(N(c4ccc5c(c4)C4(CC6CCC4C6)c4ccccc4-5)c4ccc5ccccc5c4-c4ccccc4)cc3)c21 ; CC1(C)c2ccccc2-c2cccc(-c3ccccc3-c3ccc(N(c4cccc(-c5cccc6oc7c8ccccc8ccc7c56)c4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4cccc5sc6ccccc6c45)cc3)c21]

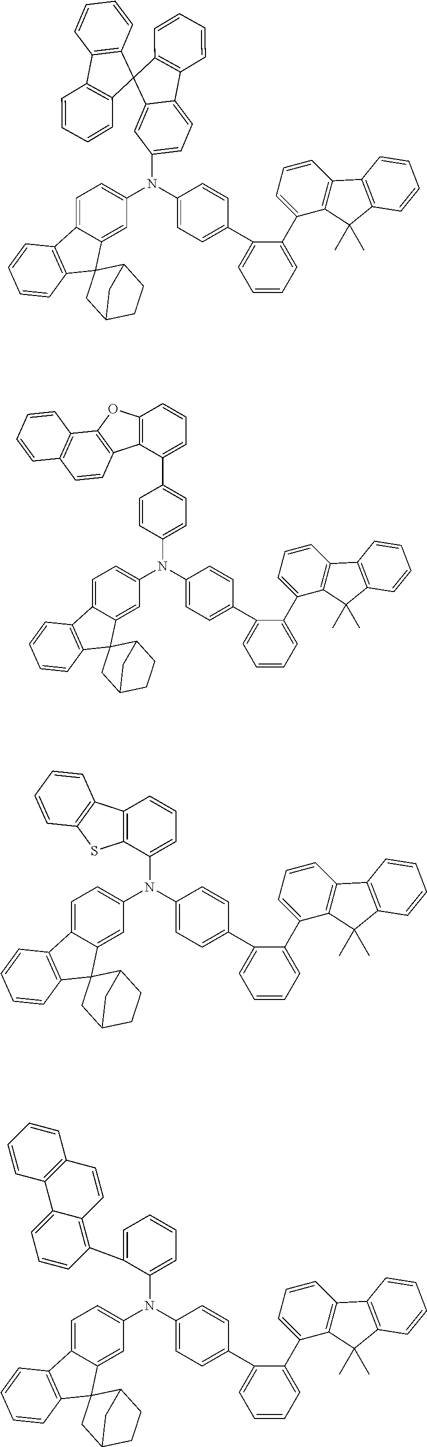 [SMILES: CC1(C)c2ccccc2-c2cccc(-c3ccccc3-c3ccc(N(c4ccc5c(c4)C4(c6ccccc6-c6ccccc64)c4ccccc4-5)c4ccc5c(c4)C4(CC6CCC4C6)c4ccccc4-5)cc3)c21 ; CC1(C)c2ccccc2-c2cccc(-c3ccccc3-c3ccc(N(c4ccc(-c5cccc6oc7c8ccccc8ccc7c56)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4cccc5c4sc4ccccc45)cc3)c21 ; CC1(C)c2ccccc2-c2cccc(-c3ccccc3-c3ccc(N(c4ccc5c(c4)C4(CC6CCC4C6)c4ccccc4-5)c4ccccc4-c4cccc5c4ccc4ccccc45)cc3)c21]

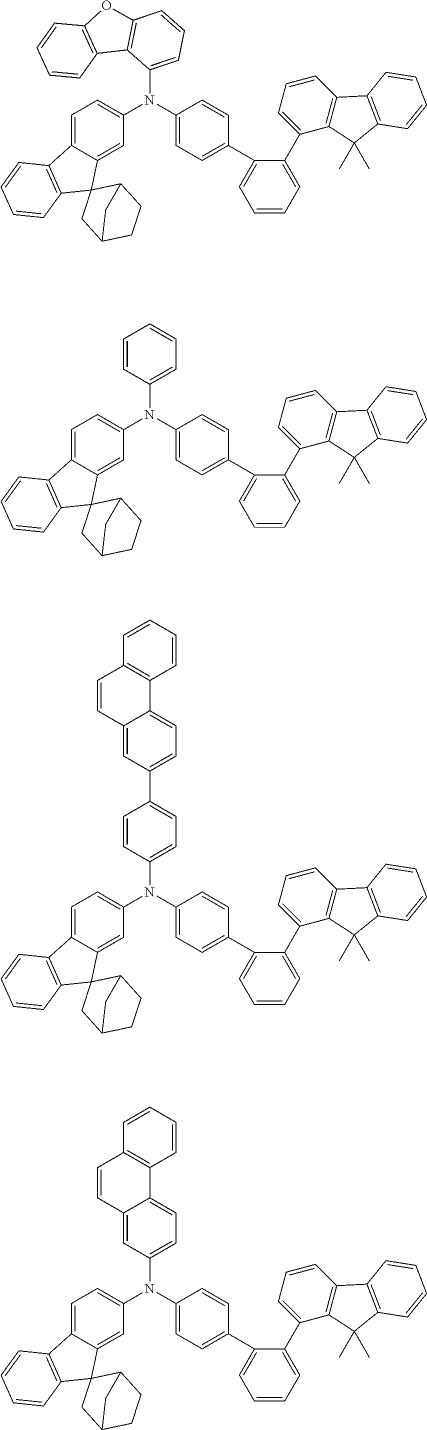 [SMILES: CC1(C)c2ccccc2-c2cccc(-c3ccccc3-c3ccc(N(c4ccc5c(c4)C4(CC6CCC4C6)c4ccccc4-5)c4cccc5oc6ccccc6c45)cc3)c21 ; CC1(C)c2ccccc2-c2cccc(-c3ccccc3-c3ccc(N(c4ccccc4)c4ccc5c(c4)C4(CC6CCC4C6)c4ccccc4-5)cc3)c21 ; CC1(C)c2ccccc2-c2cccc(-c3ccccc3-c3ccc(N(c4ccc(-c5ccc6c(ccc7ccccc76)c5)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4ccc5c(ccc6ccccc65)c4)cc3)c21]

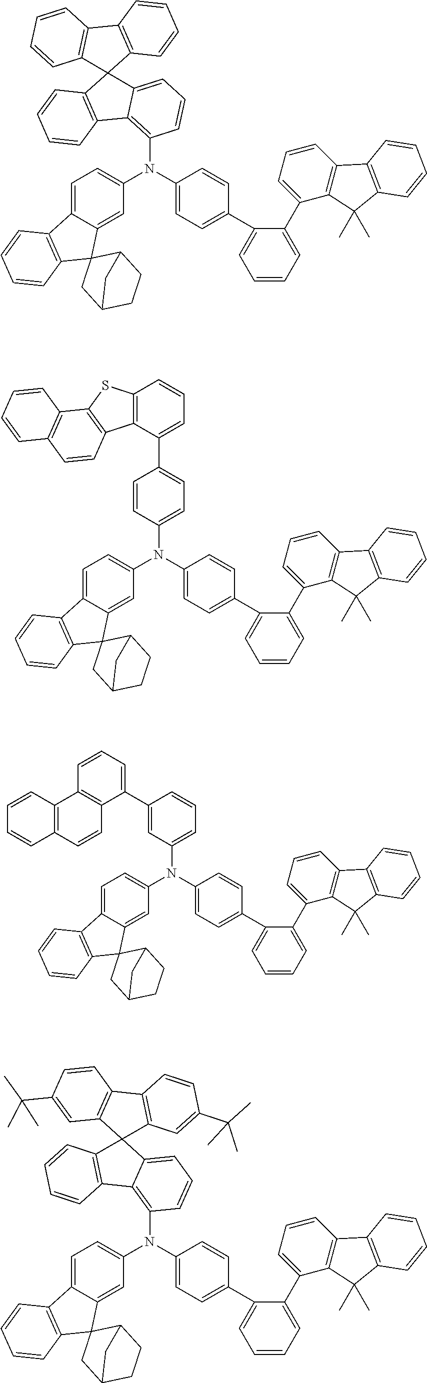

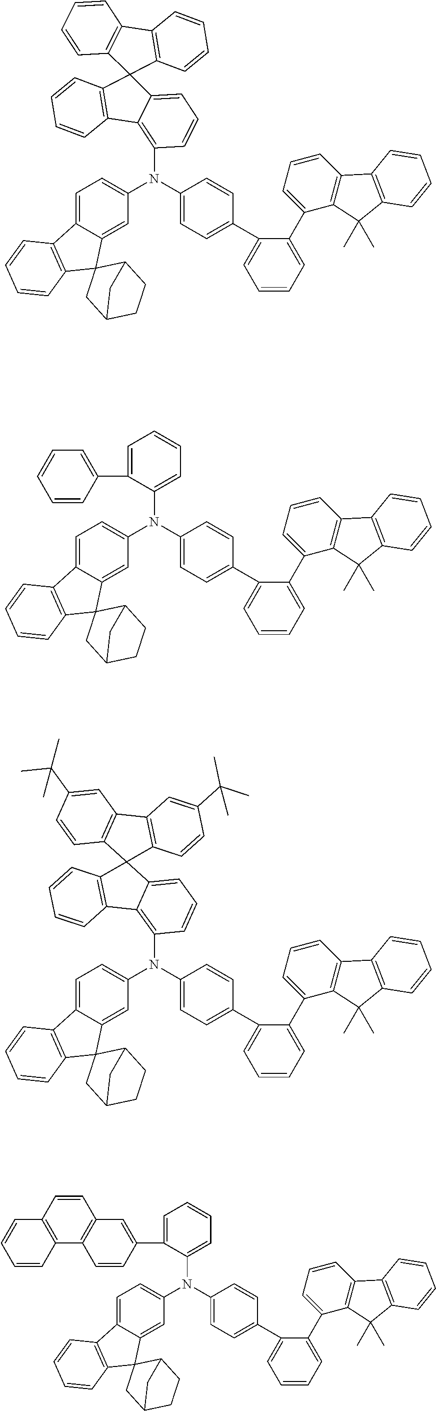 [SMILES: CC1(C)c2ccccc2-c2cccc(-c3ccccc3-c3ccc(N(c4ccc5c(c4)C4(CC6CCC4C6)c4ccccc4-5)c4cccc5c4-c4ccccc4C54c5ccccc5-c5ccccc54)cc3)c21 ; CC1(C)c2ccccc2-c2cccc(-c3ccccc3-c3ccc(N(c4ccc5c(c4)C4(CC6CCC4C6)c4ccccc4-5)c4ccccc4-c4ccccc4)cc3)c21 ; CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C3(CC5CCC3C5)c3ccccc3-4)cccc21 ; CC1(C)c2ccccc2-c2cccc(-c3ccccc3-c3ccc(N(c4ccc5c(c4)C4(CC6CCC4C6)c4ccccc4-5)c4ccccc4-c4ccc5c(ccc6ccccc65)c4)cc3)c21]

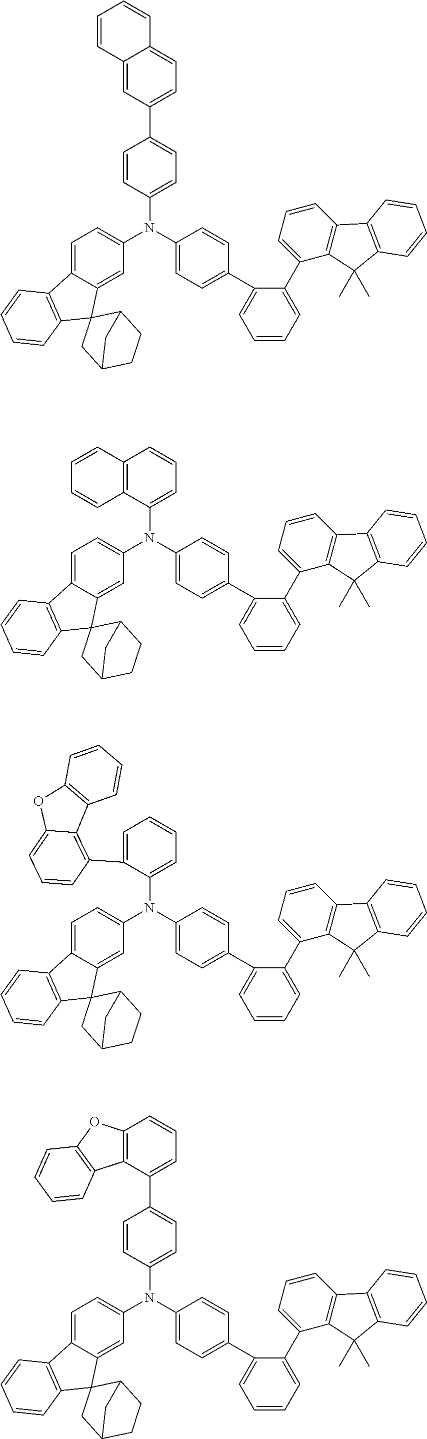 [SMILES: CC1(C)c2ccccc2-c2cccc(-c3ccccc3-c3ccc(N(c4ccc(-c5ccc6ccccc6c5)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4cccc5ccccc45)cc3)c21 ; CC1(C)c2ccccc2-c2cccc(-c3ccccc3-c3ccc(N(c4ccc5c(c4)C4(CC6CCC4C6)c4ccccc4-5)c4ccccc4-c4cccc5oc6ccccc6c45)cc3)c21 ; CC1(C)c2ccccc2-c2cccc(-c3ccccc3-c3ccc(N(c4ccc(-c5cccc6oc7ccccc7c56)cc4)c4ccc5c(c4)C4(CC6CCC4C6)c4ccccc4-5)cc3)c21]

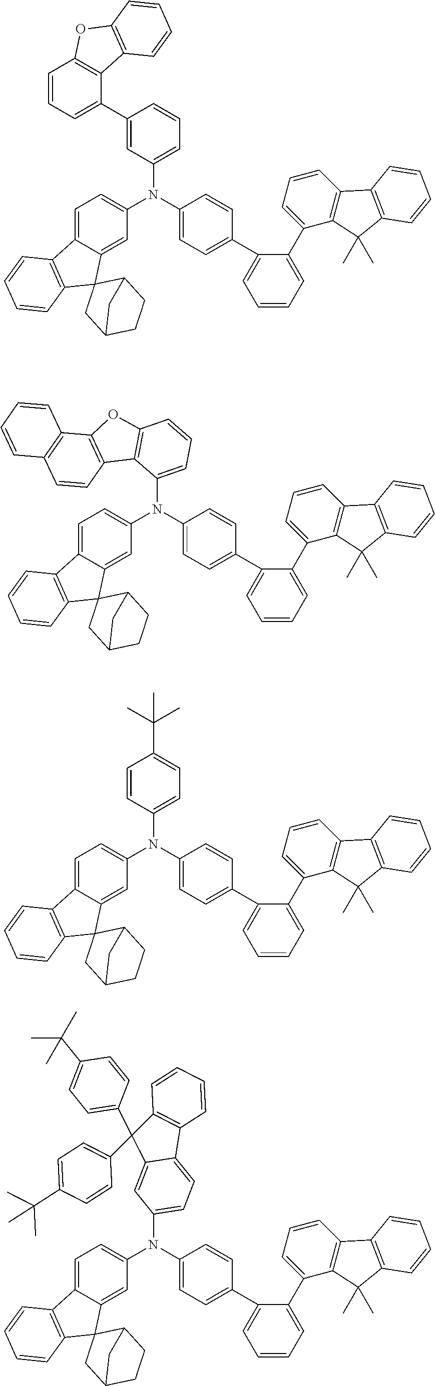

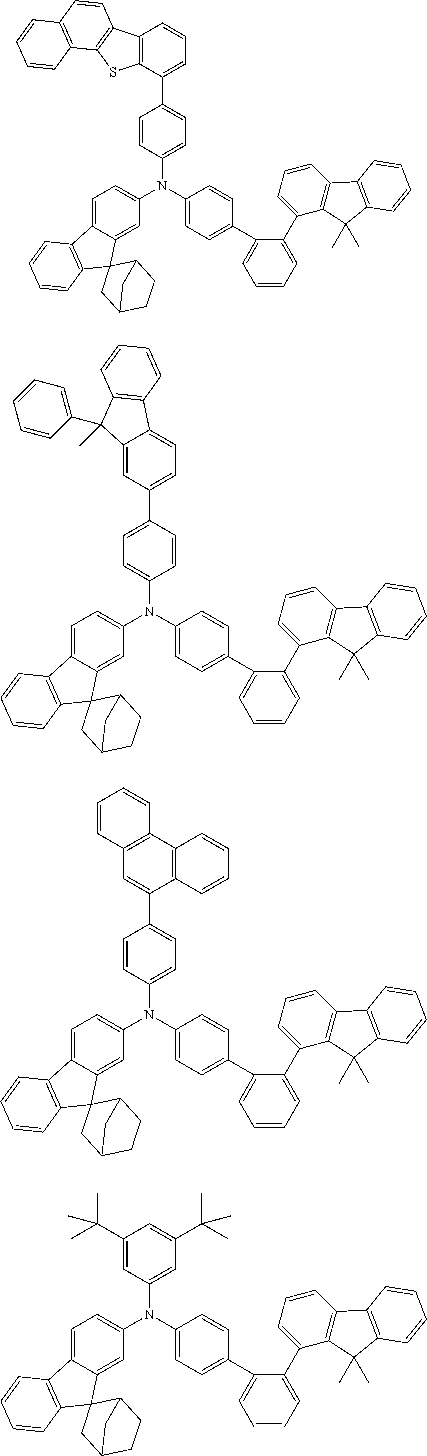

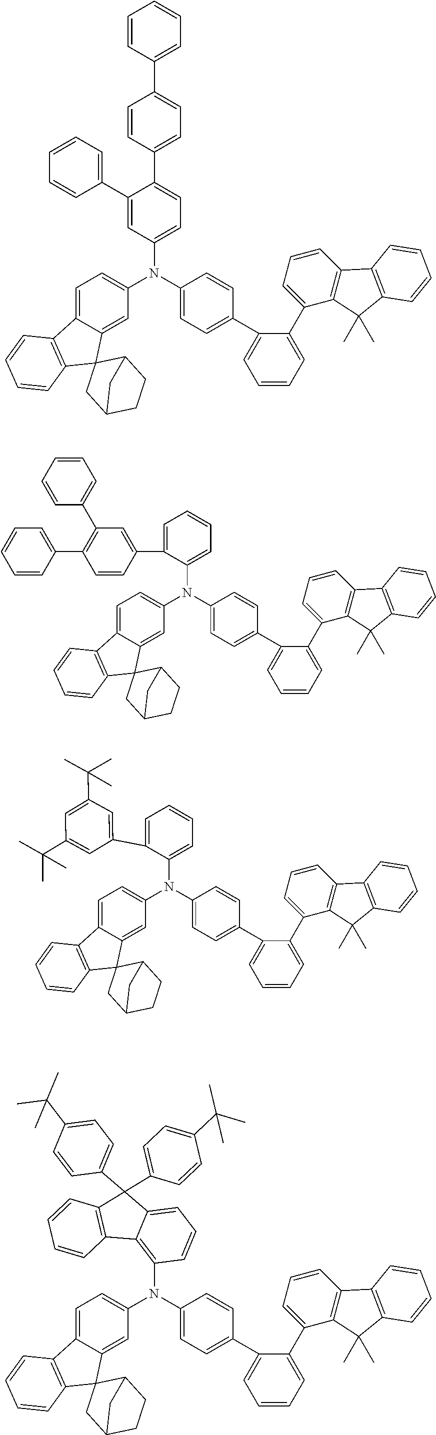

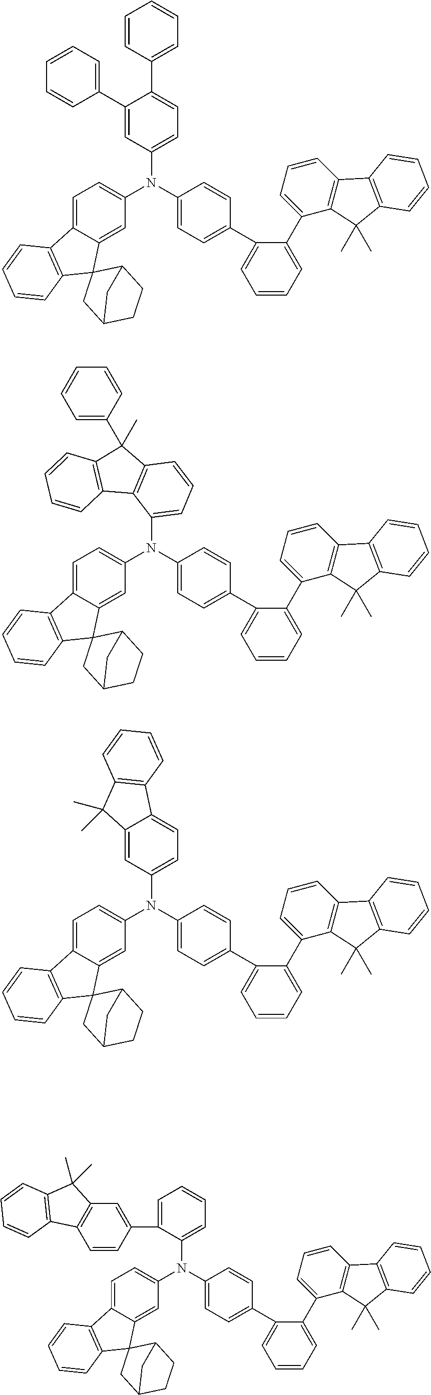 [SMILES: CC1(C)c2ccccc2-c2cccc(-c3ccccc3-c3ccc(N(c4ccc(-c5ccccc5)c(-c5ccccc5)c4)c4ccc5c(c4)C4(CC6CCC4C6)c4ccccc4-5)cc3)c21 ; CC1(C)c2ccccc2-c2cccc(-c3ccccc3-c3ccc(N(c4ccc5c(c4)C4(CC6CCC4C6)c4ccccc4-5)c4cccc5c4-c4ccccc4C5(C)c4ccccc4)cc3)c21 ; CC1(C)c2ccccc2-c2ccc(N(c3ccc(-c4ccccc4-c4cccc5c4C(C)(C)c4ccccc4-5)cc3)c3ccc4c(c3)C3(CC5CCC3C5)c3ccccc3-4)cc21 ; CC1(C)c2ccccc2-c2ccc(-c3ccccc3N(c3ccc(-c4ccccc4-c4cccc5c4C(C)(C)c4ccccc4-5)cc3)c3ccc4c(c3)C3(CC5CCC3C5)c3ccccc3-4)cc21]

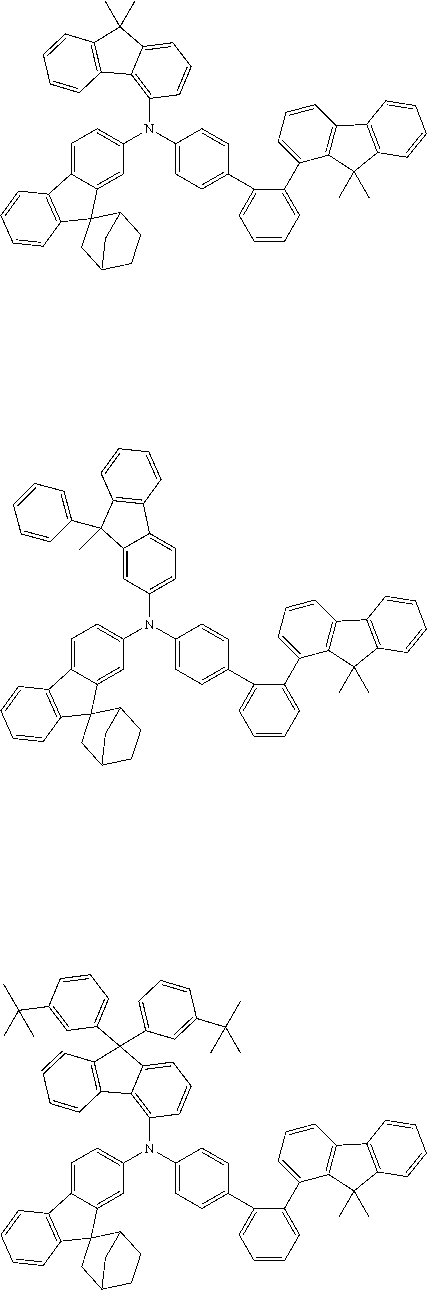 [SMILES: CC1(C)c2ccccc2-c2c(N(c3ccc(-c4ccccc4-c4cccc5c4C(C)(C)c4ccccc4-5)cc3)c3ccc4c(c3)C3(CC5CCC3C5)c3ccccc3-4)cccc21 ; CC1(C)c2ccccc2-c2cccc(-c3ccccc3-c3ccc(N(c4ccc5c(c4)C(C)(c4ccccc4)c4ccccc4-5)c4ccc5c(c4)C4(CC6CCC4C6)c4ccccc4-5)cc3)c21 ; CC(C)(C)c1cccc(C2(c3cccc(C(C)(C)C)c3)c3ccccc3-c3c(N(c4ccc(-c5ccccc5-c5cccc6c5C(C)(C)c5ccccc5-6)cc4)c4ccc5c(c4)C4(CC6CCC4C6)c4ccccc4-5)cccc32)c1]

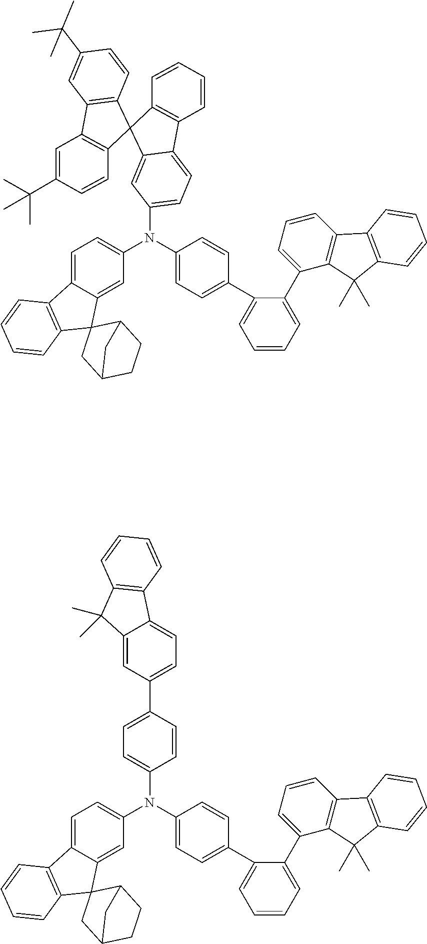 [SMILES: CC(C)(C)c1ccc2c(c1)-c1cc(C(C)(C)C)ccc1C21c2ccccc2-c2ccc(N(c3ccc(-c4ccccc4-c4cccc5c4C(C)(C)c4ccccc4-5)cc3)c3ccc4c(c3)C3(CC5CCC3C5)c3ccccc3-4)cc21 ; CC1(C)c2ccccc2-c2ccc(-c3ccc(N(c4ccc(-c5ccccc5-c5cccc6c5C(C)(C)c5ccccc5-6)cc4)c4ccc5c(c4)C4(CC6CCC4C6)c4ccccc4-5)cc3)cc21]

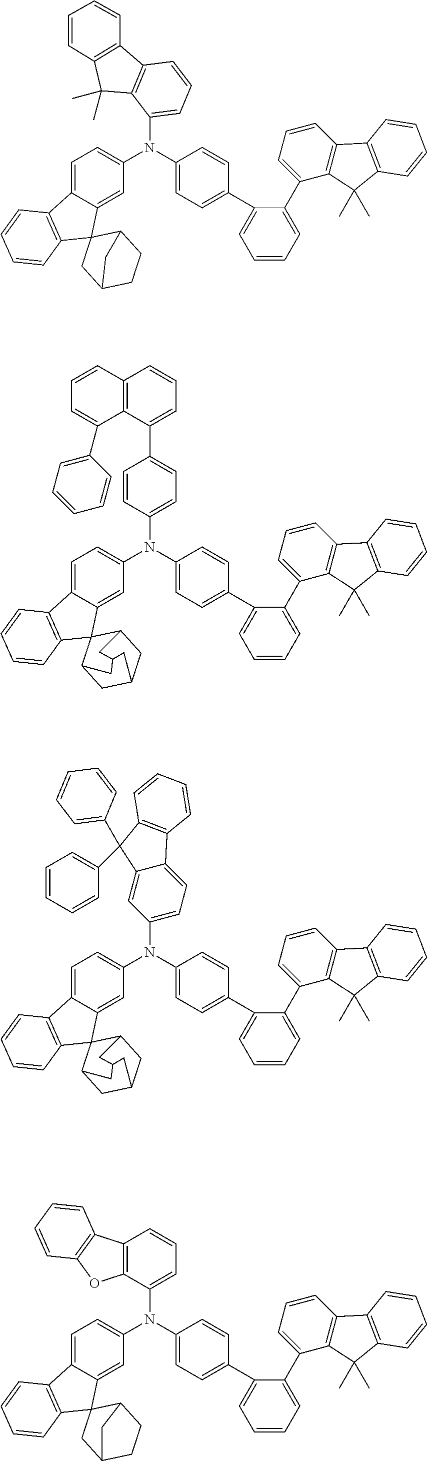 [SMILES: CC1(C)c2ccccc2-c2cccc(-c3ccccc3-c3ccc(N(c4ccc5c(c4)C4(CC6CCC4C6)c4ccccc4-5)c4cccc5c4C(C)(C)c4ccccc4-5)cc3)c21 ; CC1(C)c2ccccc2-c2cccc(-c3ccccc3-c3ccc(N(c4ccc(-c5cccc6cccc(-c7ccccc7)c56)cc4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc5c(c4)C(c4ccccc4)(c4ccccc4)c4ccccc4-5)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc5c(c4)C4(CC6CCC4C6)c4ccccc4-5)c4cccc5c4oc4ccccc45)cc3)c21]

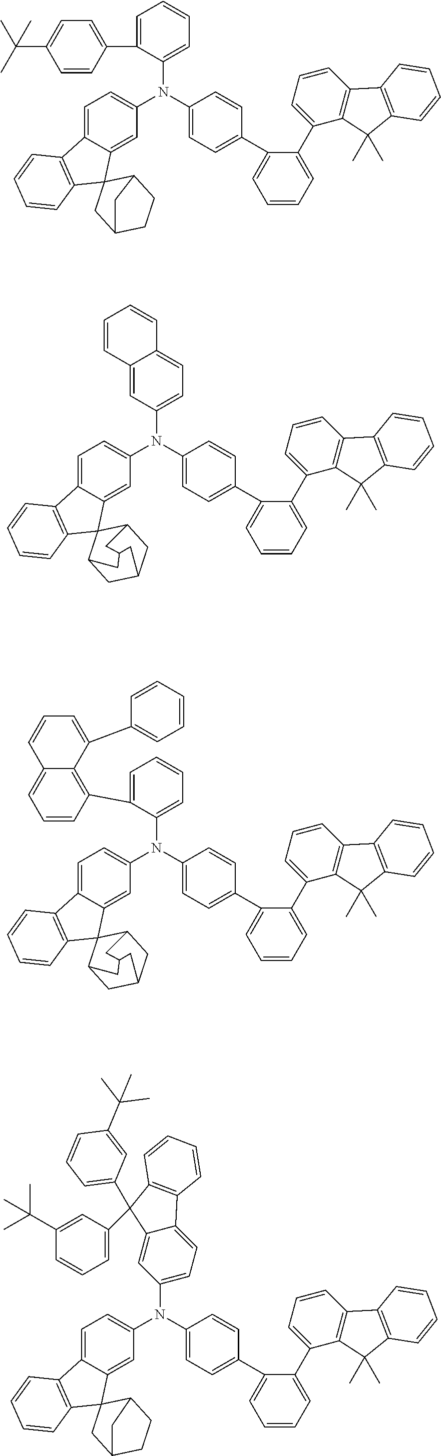 [SMILES: CC(C)(C)c1ccc(-c2ccccc2N(c2ccc(-c3ccccc3-c3cccc4c3C(C)(C)c3ccccc3-4)cc2)c2ccc3c(c2)C2(CC4CCC2C4)c2ccccc2-3)cc1 ; CC1(C)c2ccccc2-c2cccc(-c3ccccc3-c3ccc(N(c4ccc5c(c4)C4(c6ccccc6-5)C5CC6CC(C5)CC4C6)c4ccc5ccccc5c4)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4ccccc4-c4cccc5cccc(-c6ccccc6)c45)cc3)c21 ; CC(C)(C)c1cccc(C2(c3cccc(C(C)(C)C)c3)c3ccccc3-c3ccc(N(c4ccc(-c5ccccc5-c5cccc6c5C(C)(C)c5ccccc5-6)cc4)c4ccc5c(c4)C4(CC6CCC4C6)c4ccccc4-5)cc32)c1]

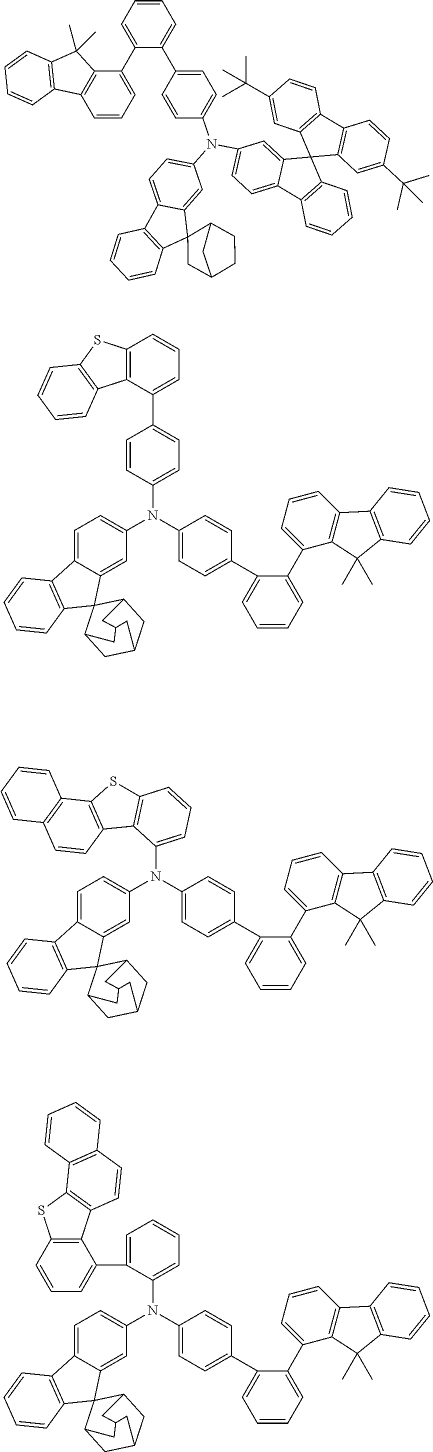

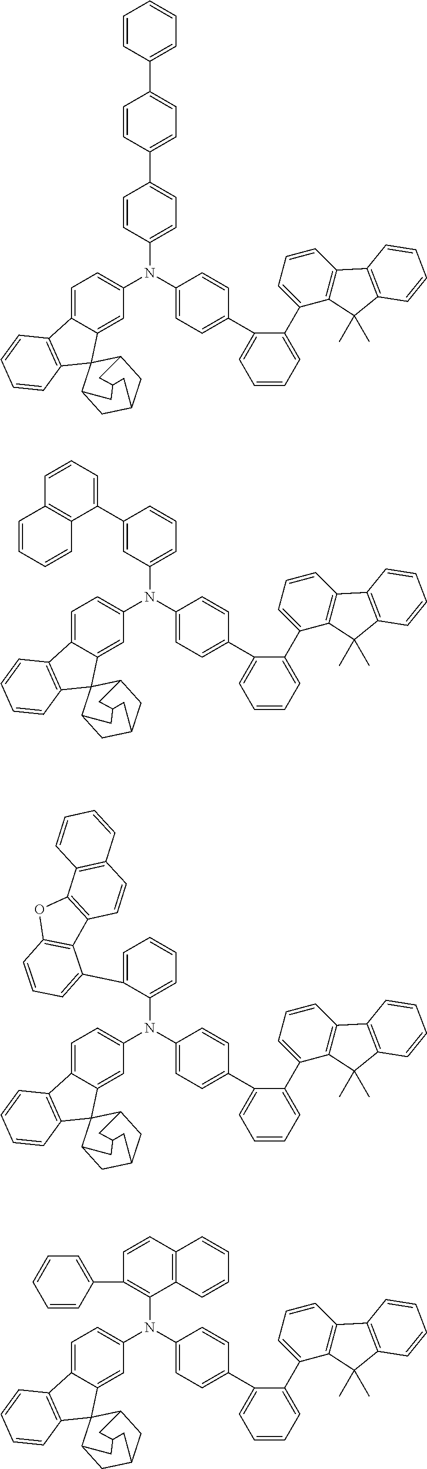 [SMILES: CC1(C)c2ccccc2-c2cccc(-c3ccccc3-c3ccc(N(c4ccc(-c5ccc(-c6ccccc6)cc5)cc4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4cccc(-c5cccc6ccccc56)c4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4ccccc4-c4cccc5oc6c7ccccc7ccc6c45)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4c(-c5ccccc5)ccc5ccccc45)cc3)c21]

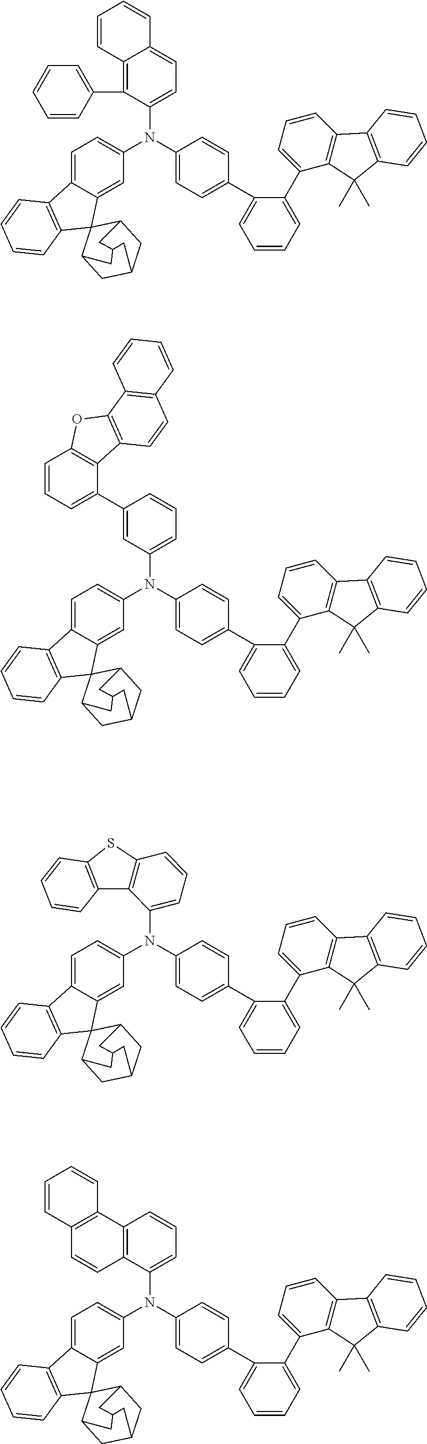 [SMILES: CC1(C)c2ccccc2-c2cccc(-c3ccccc3-c3ccc(N(c4ccc5c(c4)C4(c6ccccc6-5)C5CC6CC(C5)CC4C6)c4ccc5ccccc5c4-c4ccccc4)cc3)c21 ; CC1(C)c2ccccc2-c2cccc(-c3ccccc3-c3ccc(N(c4cccc(-c5cccc6oc7c8ccccc8ccc7c56)c4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4cccc5sc6ccccc6c45)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4cccc5c4ccc4ccccc45)cc3)c21]

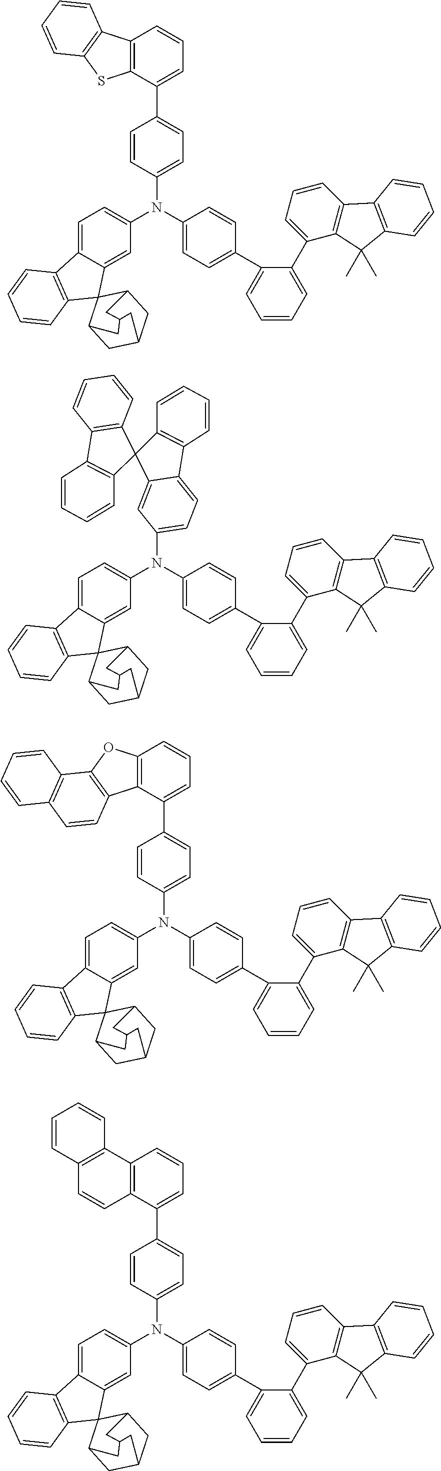

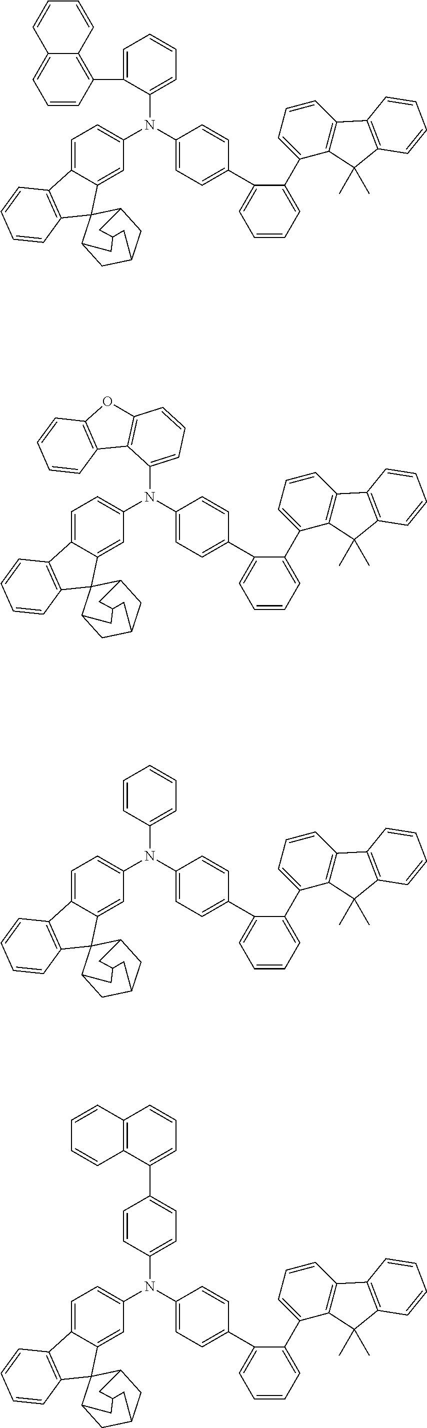 [SMILES: CC1(C)c2ccccc2-c2cccc(-c3ccccc3-c3ccc(N(c4ccc5c(c4)C4(c6ccccc6-5)C5CC6CC(C5)CC4C6)c4ccccc4-c4cccc5ccccc45)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4cccc5oc6ccccc6c45)cc3)c21 ; CC1(C)c2ccccc2-c2cccc(-c3ccccc3-c3ccc(N(c4ccccc4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc(-c5cccc6ccccc56)cc4)c4ccc5c(c4)C4(c6ccccc6-5)C5CC6CC(C5)CC4C6)cc3)c21]

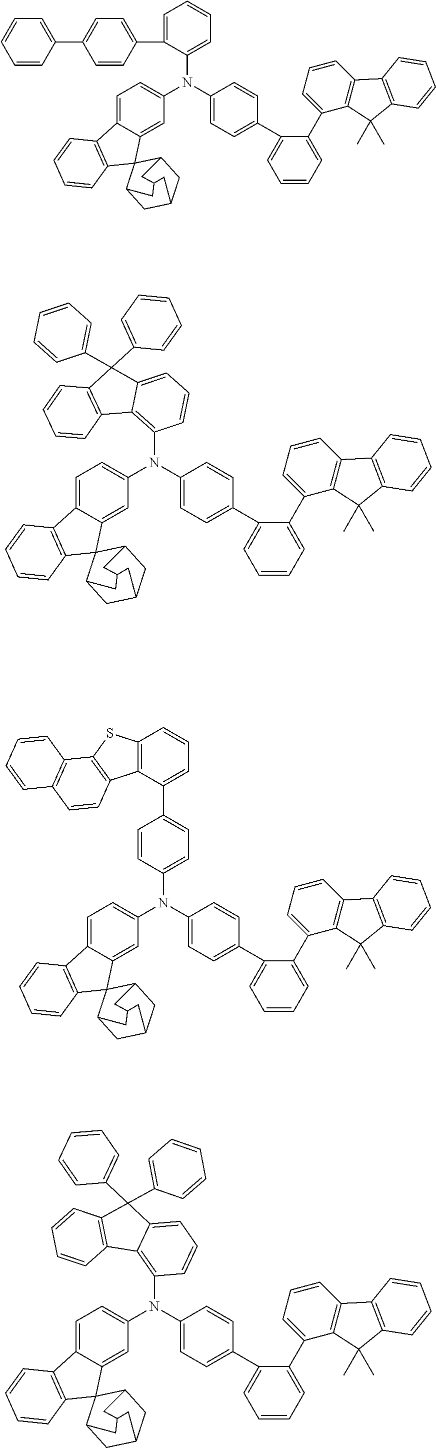

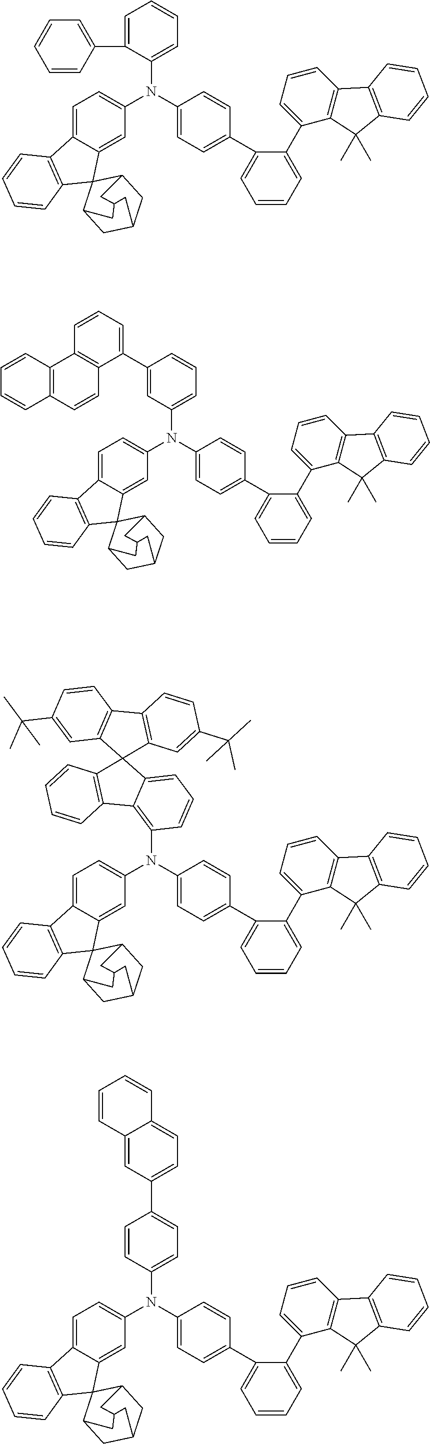

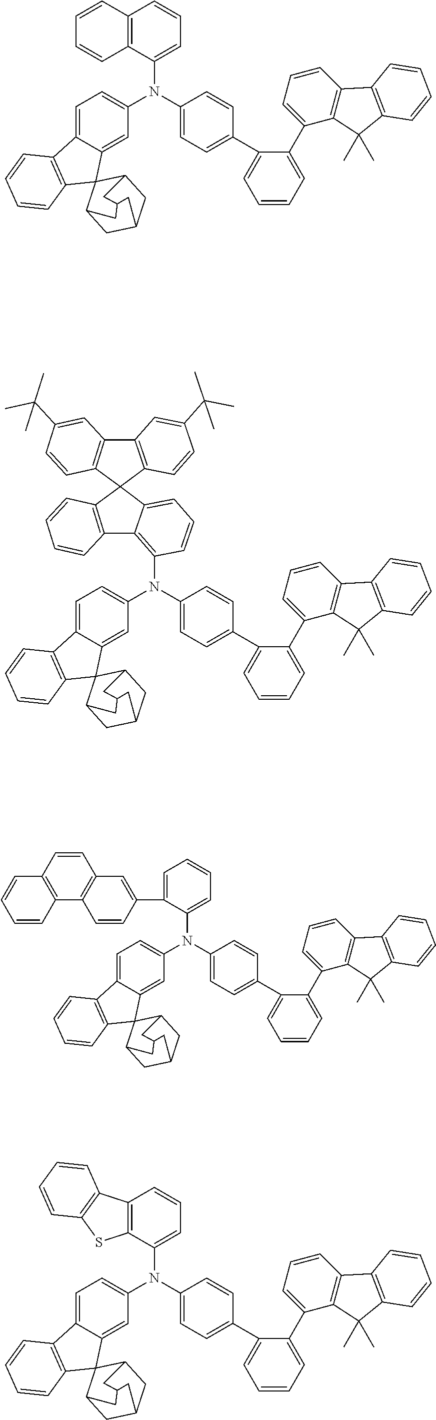 [SMILES: CC1(C)c2ccccc2-c2cccc(-c3ccccc3-c3ccc(N(c4ccc5c(c4)C4(c6ccccc6-5)C5CC6CC(C5)CC4C6)c4cccc5ccccc45)cc3)c21 ; CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C3(c5ccccc5-4)C4CC5CC(C4)CC3C5)cccc21 ; CC1(C)c2ccccc2-c2cccc(-c3ccccc3-c3ccc(N(c4ccc5c(c4)C4(c6ccccc6-5)C5CC6CC(C5)CC4C6)c4ccccc4-c4ccc5c(ccc6ccccc65)c4)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4cccc5c4sc4ccccc45)cc3)c21]

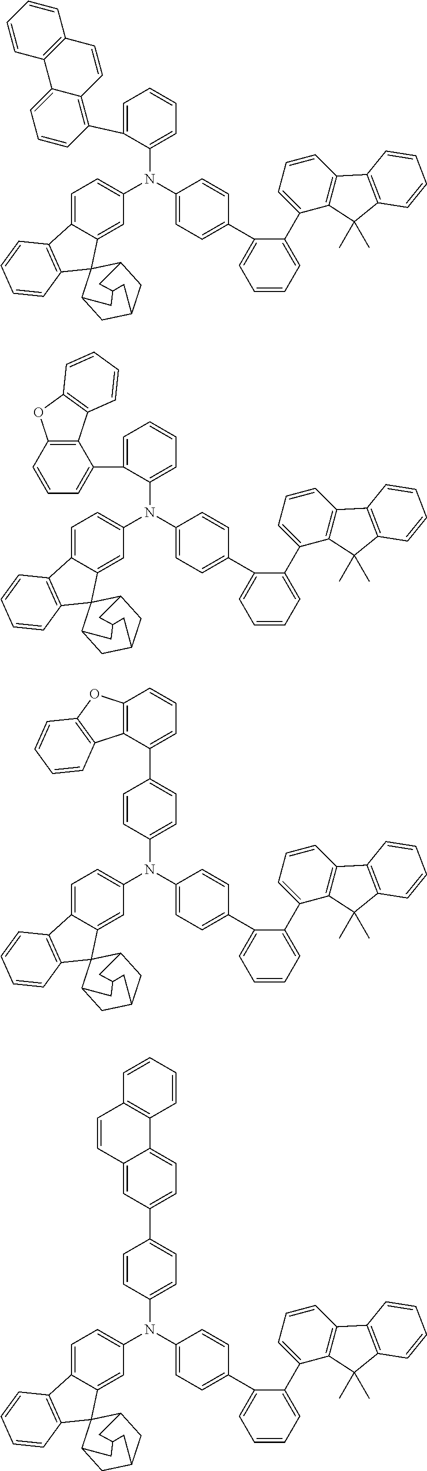

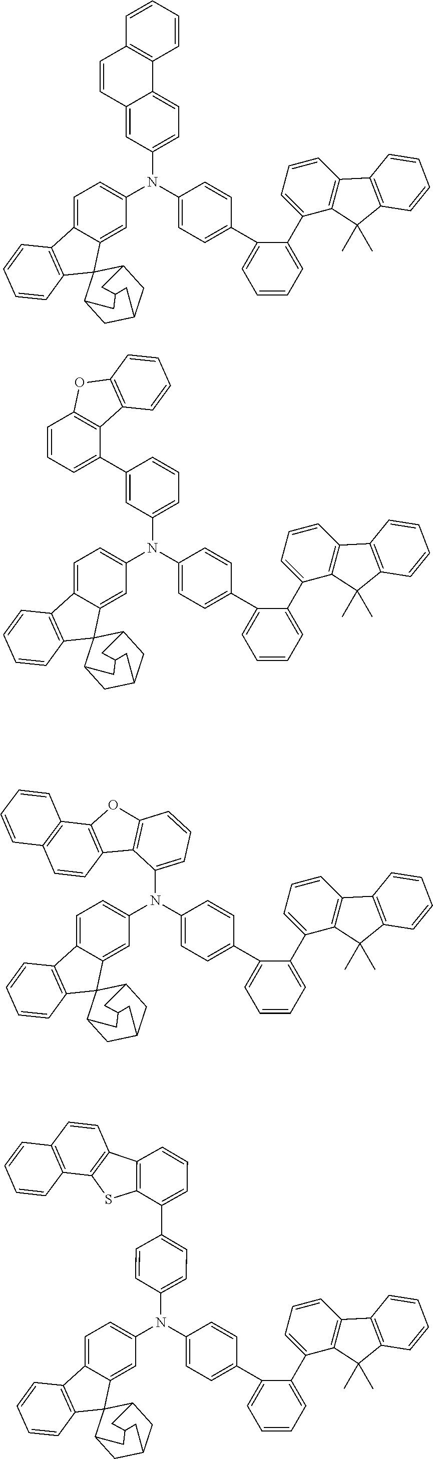

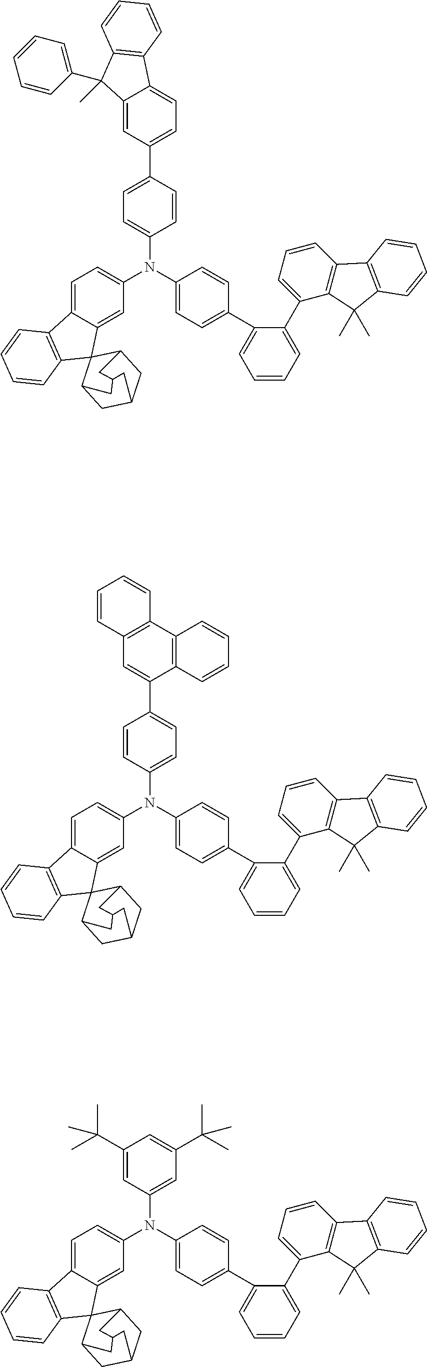 [SMILES: CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc(-c5cc6ccccc6c6ccccc56)cc4)c4ccc5c(c4)C4(c6ccccc6-5)C5CC6CC(C5)CC4C6)cc3)c21 ; CC(C)(C)c1cc(N(c2ccc(-c3ccccc3-c3cccc4c3C(C)(C)c3ccccc3-4)cc2)c2ccc3c(c2)C2(c4ccccc4-3)C3CC4CC(C3)CC2C4)cc(C(C)(C)C)c1]

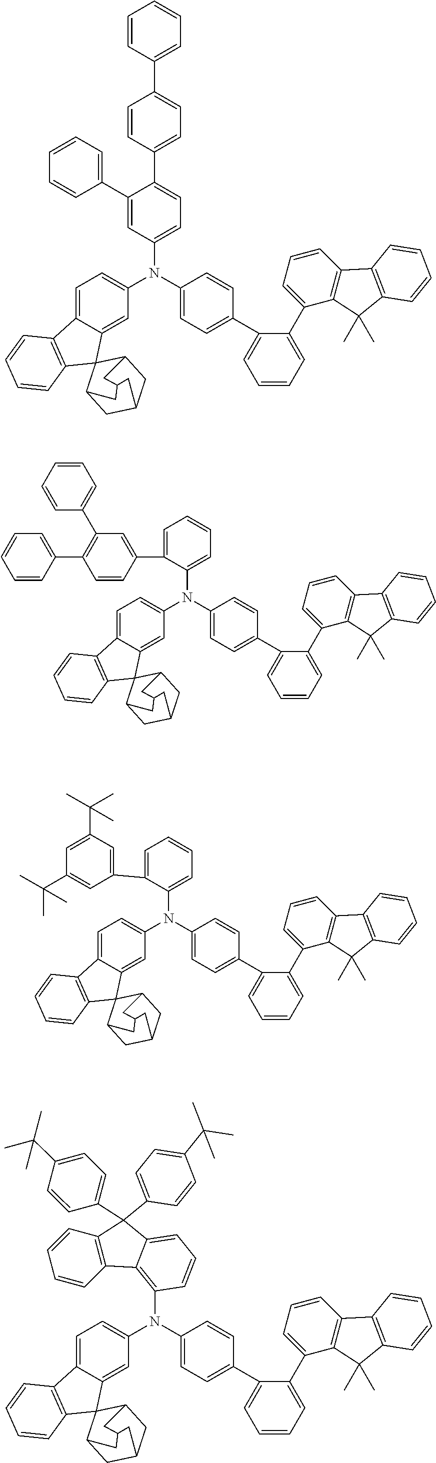

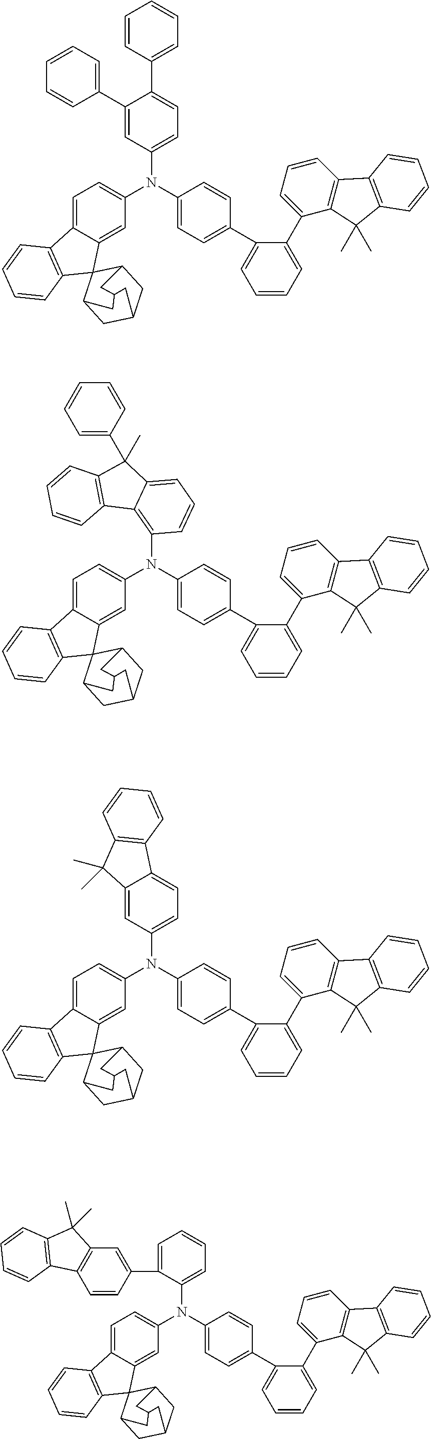 [SMILES: CC1(C)c2ccccc2-c2cccc(-c3ccccc3-c3ccc(N(c4ccc(-c5ccccc5)c(-c5ccccc5)c4)c4ccc5c(c4)C4(c6ccccc6-5)C5CC6CC(C5)CC4C6)cc3)c21 ; CC1(C)c2ccccc2-c2cccc(-c3ccccc3-c3ccc(N(c4ccc5c(c4)C4(c6ccccc6-5)C5CC6CC(C5)CC4C6)c4cccc5c4-c4ccccc4C5(C)c4ccccc4)cc3)c21 ; CC1(C)c2ccccc2-c2ccc(N(c3ccc(-c4ccccc4-c4cccc5c4C(C)(C)c4ccccc4-5)cc3)c3ccc4c(c3)C3(c5ccccc5-4)C4CC5CC(C4)CC3C5)cc21 ; CC1(C)c2ccccc2-c2ccc(-c3ccccc3N(c3ccc(-c4ccccc4-c4cccc5c4C(C)(C)c4ccccc4-5)cc3)c3ccc4c(c3)C3(c5ccccc5-4)C4CC5CC(C4)CC3C5)cc21]

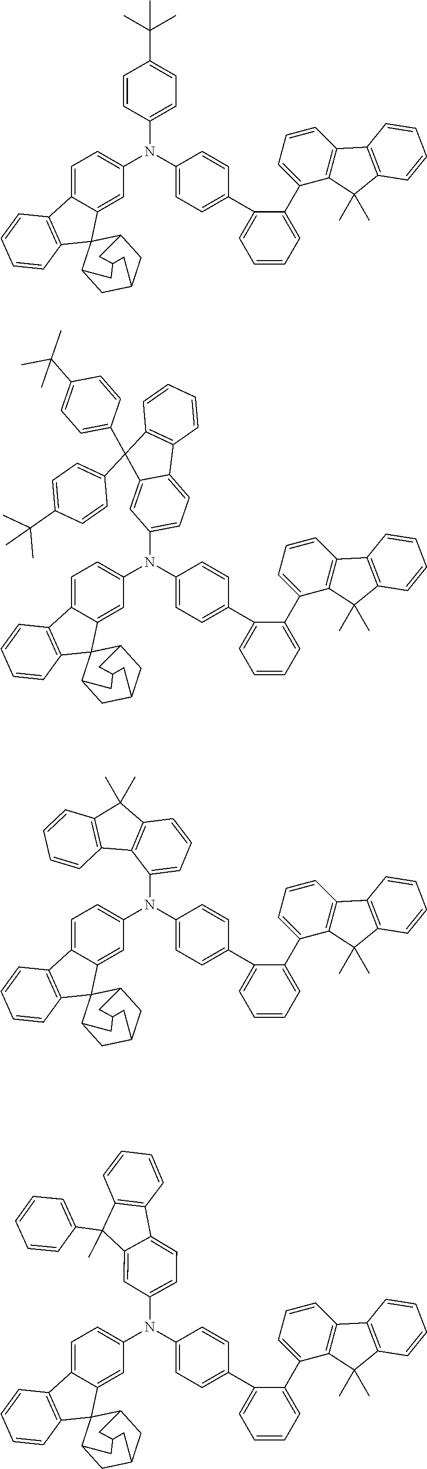

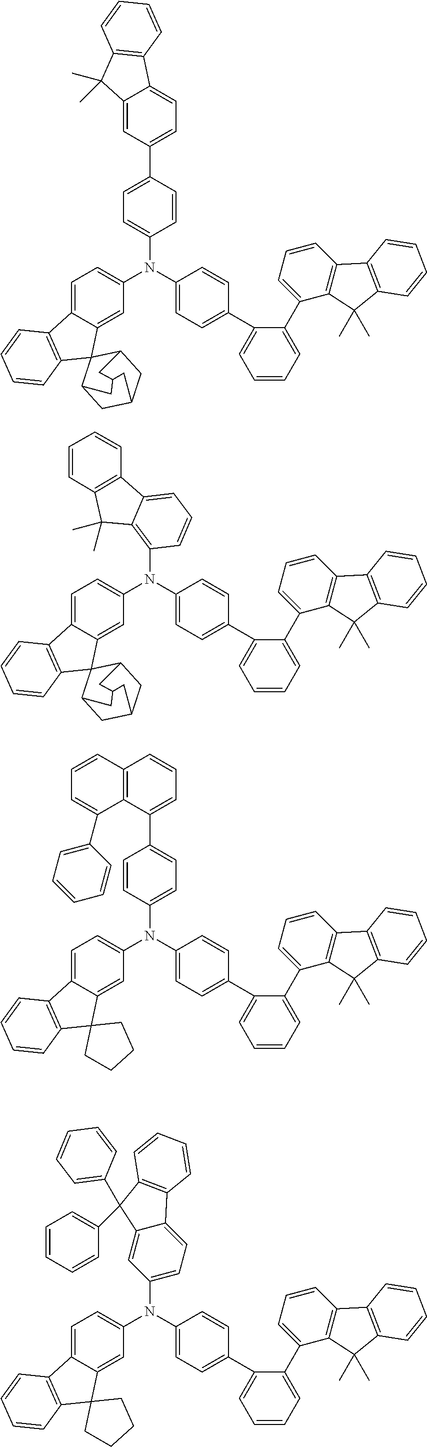

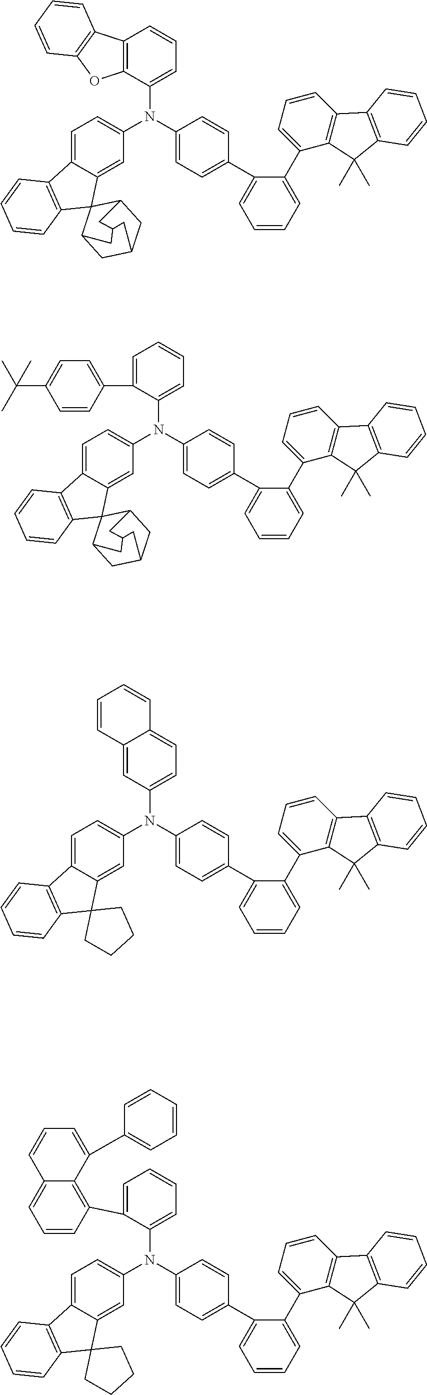 [SMILES: CC1(C)c2ccccc2-c2cccc(-c3ccccc3-c3ccc(N(c4ccc5c(c4)C4(c6ccccc6-5)C5CC6CC(C5)CC4C6)c4cccc5c4oc4ccccc45)cc3)c21 ; CC(C)(C)c1ccc(-c2ccccc2N(c2ccc(-c3ccccc3-c3cccc4c3C(C)(C)c3ccccc3-4)cc2)c2ccc3c(c2)C2(c4ccccc4-3)C3CC4CC(C3)CC2C4)cc1 ; CC1(C)c2ccccc2-c2cccc(-c3ccccc3-c3ccc(N(c4ccc5c(c4)C4(CCCC4)c4ccccc4-5)c4ccc5ccccc5c4)cc3)c21 ; CC1(C)c2ccccc2-c2cccc(-c3ccccc3-c3ccc(N(c4ccc5c(c4)C4(CCCC4)c4ccccc4-5)c4ccccc4-c4cccc5cccc(-c6ccccc6)c45)cc3)c21]

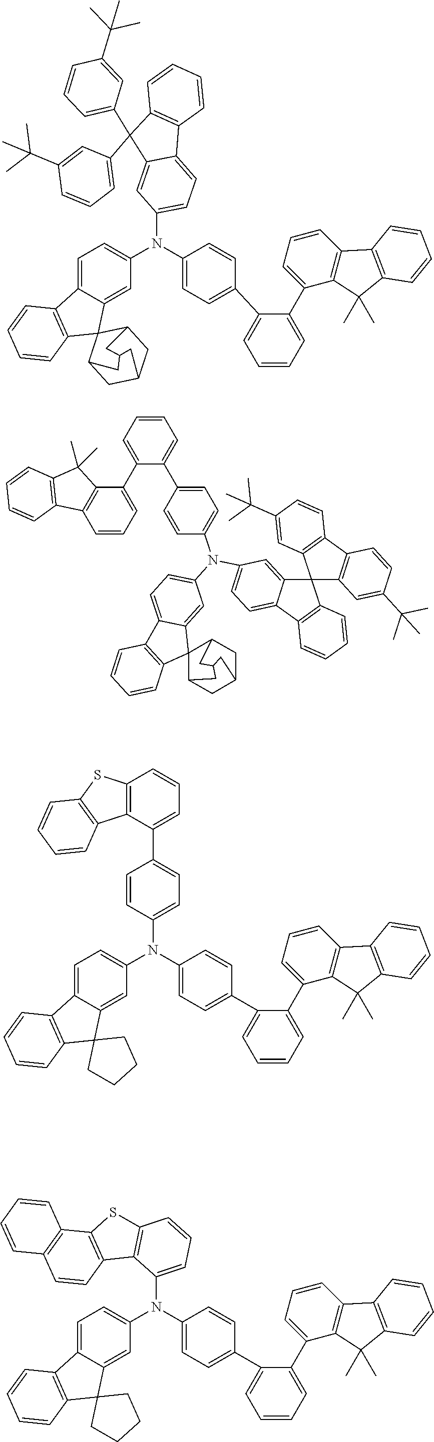

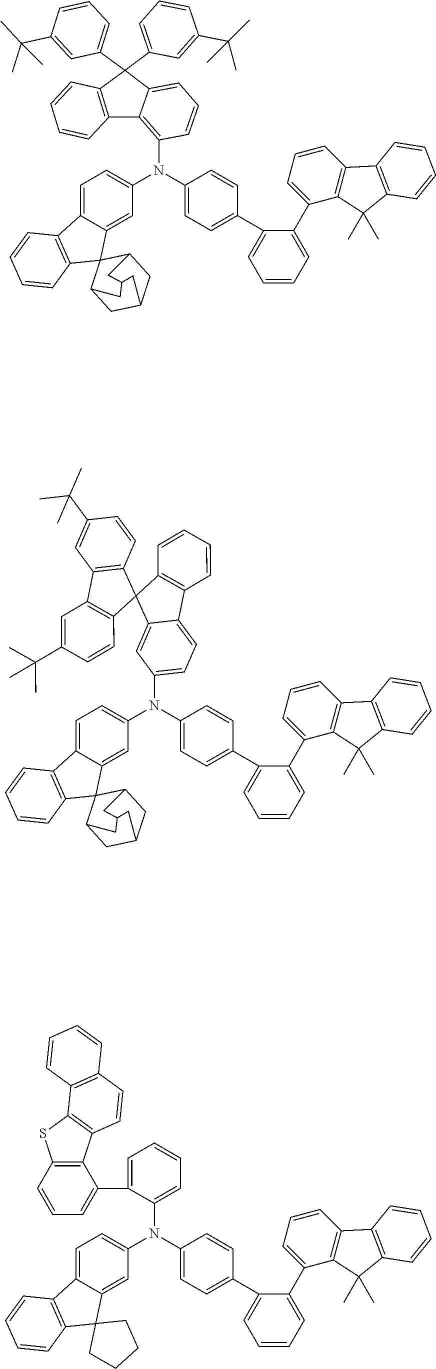 [SMILES: CC(C)(C)c1cccc(C2(c3cccc(C(C)(C)C)c3)c3ccccc3-c3c(N(c4ccc(-c5ccccc5-c5cccc6c5C(C)(C)c5ccccc5-6)cc4)c4ccc5c(c4)C4(c6ccccc6-5)C5CC6CC(C5)CC4C6)cccc32)c1 ; CC(C)(C)c1ccc2c(c1)-c1cc(C(C)(C)C)ccc1C21c2ccccc2-c2ccc(N(c3ccc(-c4ccccc4-c4cccc5c4C(C)(C)c4ccccc4-5)cc3)c3ccc4c(c3)C3(c5ccccc5-4)C4CC5CC(C4)CC3C5)cc21 ; CC1(C)c2ccccc2-c2cccc(-c3ccccc3-c3ccc(N(c4ccc5c(c4)C4(CCCC4)c4ccccc4-5)c4ccccc4-c4cccc5sc6c7ccccc7ccc6c45)cc3)c21]

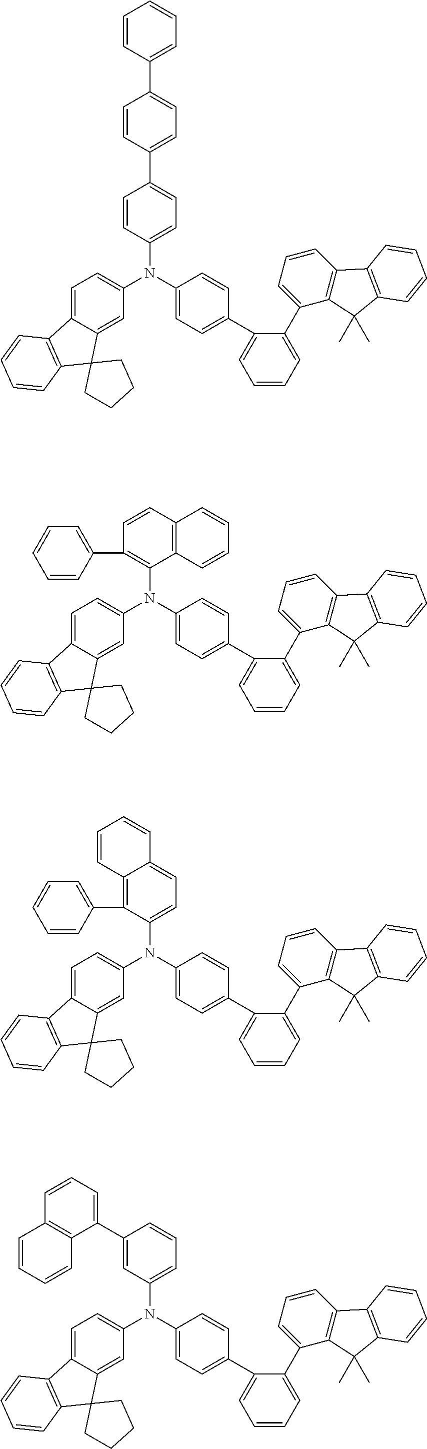 [SMILES: CC1(C)c2ccccc2-c2cccc(-c3ccccc3-c3ccc(N(c4ccc(-c5ccc(-c6ccccc6)cc5)cc4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4c(-c5ccccc5)ccc5ccccc45)cc3)c21 ; CC1(C)c2ccccc2-c2cccc(-c3ccccc3-c3ccc(N(c4ccc5c(c4)C4(CCCC4)c4ccccc4-5)c4ccc5ccccc5c4-c4ccccc4)cc3)c21 ; CC1(C)c2ccccc2-c2cccc(-c3ccccc3-c3ccc(N(c4cccc(-c5cccc6ccccc56)c4)c4ccc5c(c4)C4(CCCC4)c4ccccc4-5)cc3)c21]

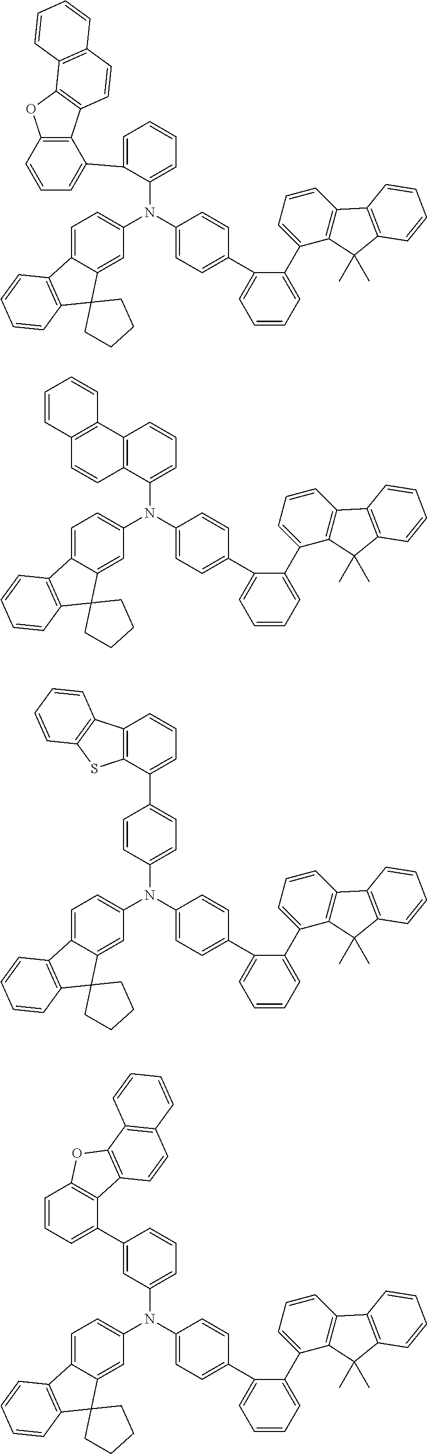 [SMILES: CC1(C)c2ccccc2-c2cccc(-c3ccccc3-c3ccc(N(c4ccc5c(c4)C4(CCCC4)c4ccccc4-5)c4ccccc4-c4cccc5oc6c7ccccc7ccc6c45)cc3)c21 ; CC1(C)c2ccccc2-c2cccc(-c3ccccc3-c3ccc(N(c4ccc5c(c4)C4(CCCC4)c4ccccc4-5)c4cccc5c4ccc4ccccc45)cc3)c21 ; CC1(C)c2ccccc2-c2cccc(-c3ccccc3-c3ccc(N(c4ccc(-c5cccc6c5sc5ccccc56)cc4)c4ccc5c(c4)C4(CCCC4)c4ccccc4-5)cc3)c21 ; CC1(C)c2ccccc2-c2cccc(-c3ccccc3-c3ccc(N(c4cccc(-c5cccc6oc7c8ccccc8ccc7c56)c4)c4ccc5c(c4)C4(CCCC4)c4ccccc4-5)cc3)c21]

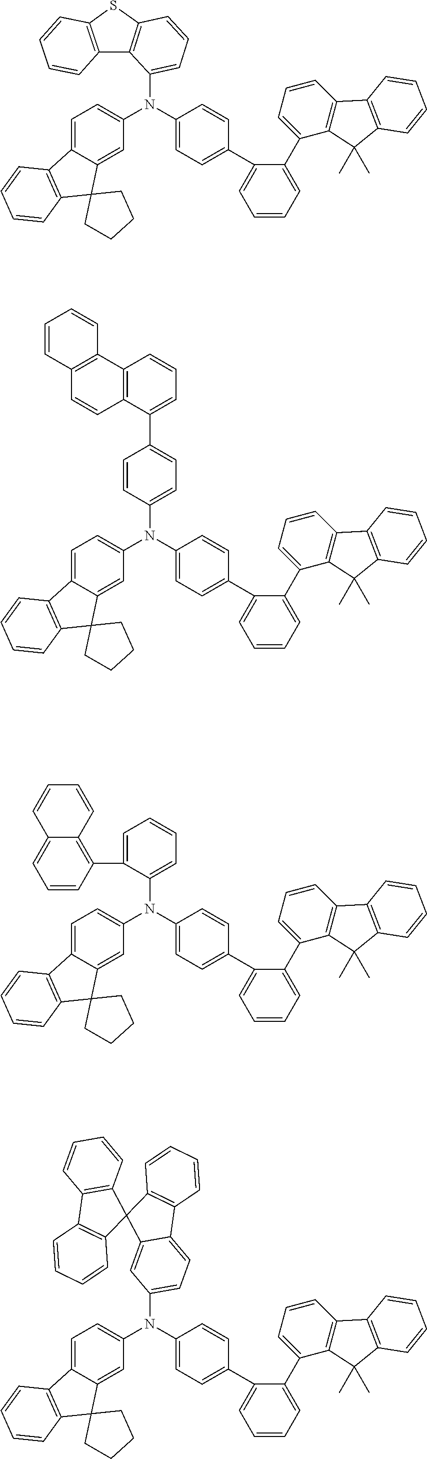 [SMILES: CC1(C)c2ccccc2-c2cccc(-c3ccccc3-c3ccc(N(c4ccc5c(c4)C4(CCCC4)c4ccccc4-5)c4cccc5sc6ccccc6c45)cc3)c21 ; CC1(C)c2ccccc2-c2cccc(-c3ccccc3-c3ccc(N(c4ccc(-c5cccc6c5ccc5ccccc56)cc4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4ccccc4-c4cccc5ccccc45)cc3)c21 ; CC1(C)c2ccccc2-c2cccc(-c3ccccc3-c3ccc(N(c4ccc5c(c4)C4(CCCC4)c4ccccc4-5)c4ccc5c(c4)C4(c6ccccc6-c6ccccc64)c4ccccc4-5)cc3)c21]

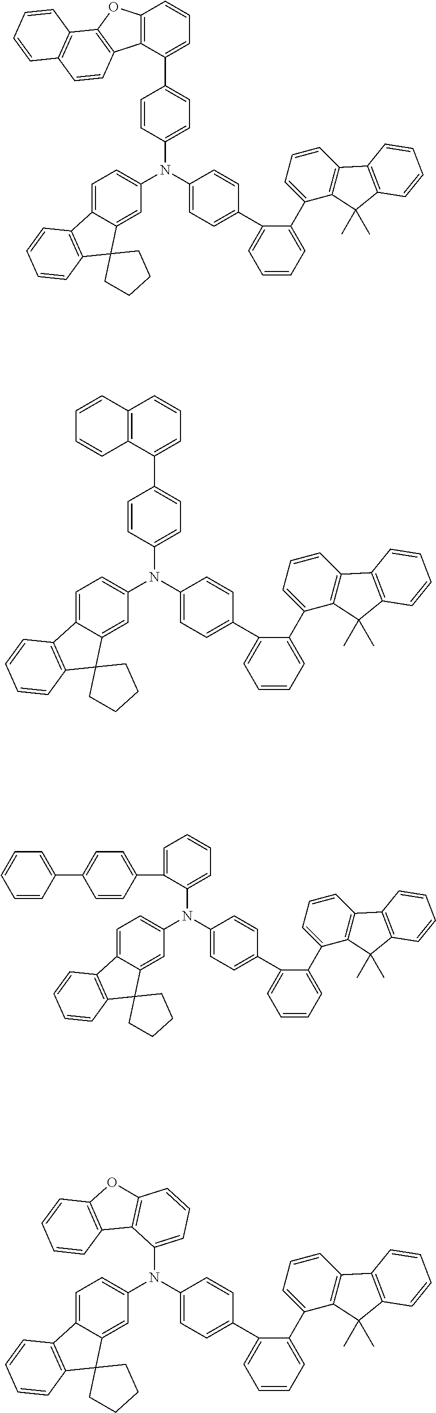 [SMILES: CC1(C)c2ccccc2-c2cccc(-c3ccccc3-c3ccc(N(c4ccc(-c5cccc6oc7c8ccccc8ccc7c56)cc4)c4ccc5c(c4)C4(CCCC4)c4ccccc4-5)cc3)c21 ; CC1(C)c2ccccc2-c2cccc(-c3ccccc3-c3ccc(N(c4ccc(-c5cccc6ccccc56)cc4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4ccccc4-c4ccc(-c5ccccc5)cc4)cc3)c21 ; CC1(C)c2ccccc2-c2cccc(-c3ccccc3-c3ccc(N(c4ccc5c(c4)C4(CCCC4)c4ccccc4-5)c4cccc5oc6ccccc6c45)cc3)c21]

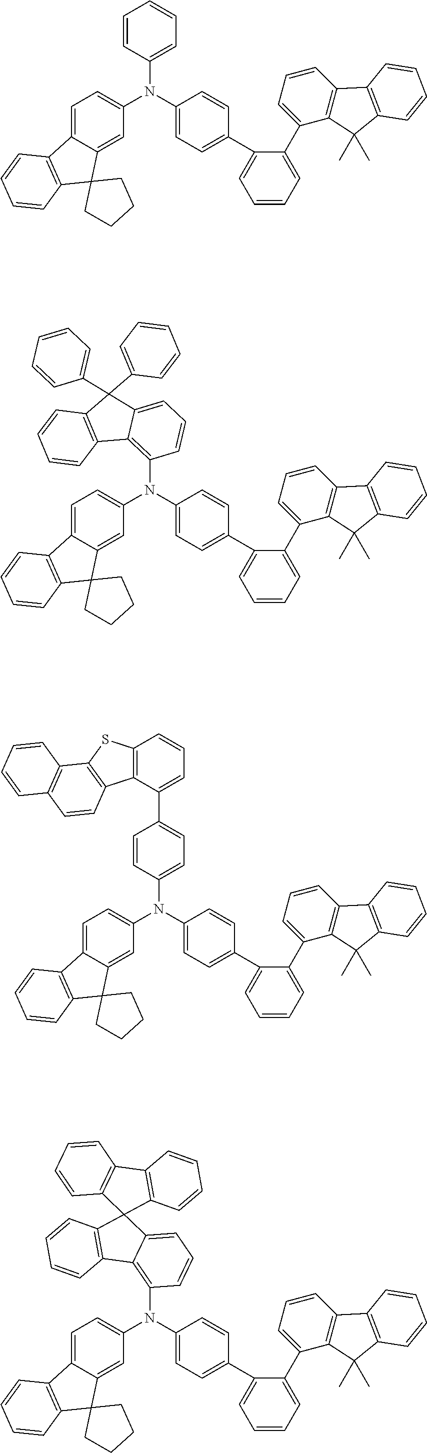 [SMILES: CC1(C)c2ccccc2-c2cccc(-c3ccccc3-c3ccc(N(c4ccccc4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4cccc5c4-c4ccccc4C5(c4ccccc4)c4ccccc4)cc3)c21 ; CC1(C)c2ccccc2-c2cccc(-c3ccccc3-c3ccc(N(c4ccc(-c5cccc6sc7c8ccccc8ccc7c56)cc4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4cccc5c4-c4ccccc4C54c5ccccc5-c5ccccc54)cc3)c21]

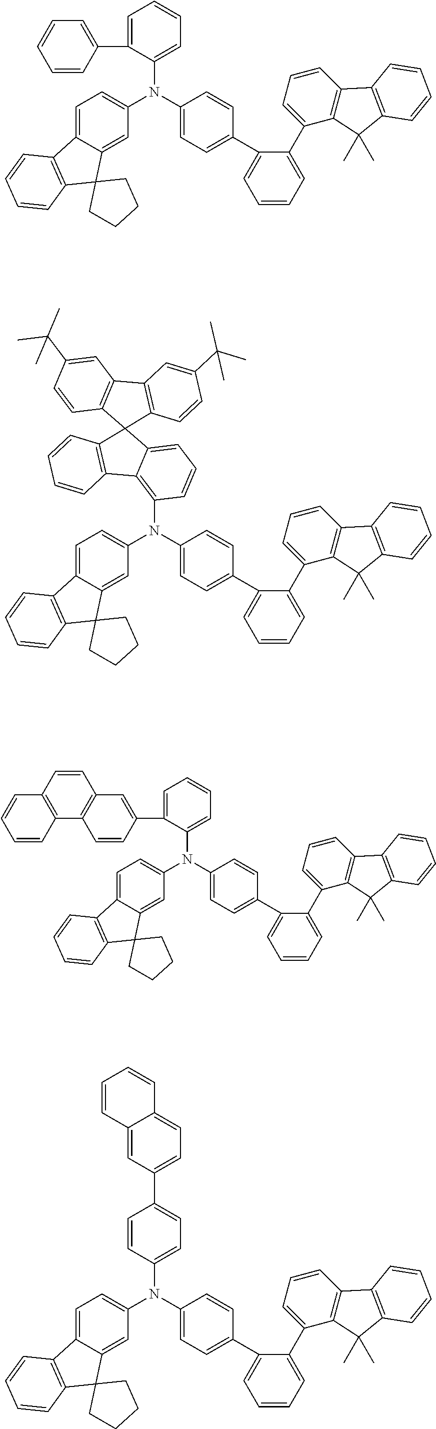 [SMILES: CC1(C)c2ccccc2-c2cccc(-c3ccccc3-c3ccc(N(c4ccc5c(c4)C4(CCCC4)c4ccccc4-5)c4ccccc4-c4ccccc4)cc3)c21 ; CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C3(CCCC3)c3ccccc3-4)cccc21 ; CC1(C)c2ccccc2-c2cccc(-c3ccccc3-c3ccc(N(c4ccc5c(c4)C4(CCCC4)c4ccccc4-5)c4ccccc4-c4ccc5c(ccc6ccccc65)c4)cc3)c21 ; CC1(C)c2ccccc2-c2cccc(-c3ccccc3-c3ccc(N(c4ccc(-c5ccc6ccccc6c5)cc4)c4ccc5c(c4)C4(CCCC4)c4ccccc4-5)cc3)c21]

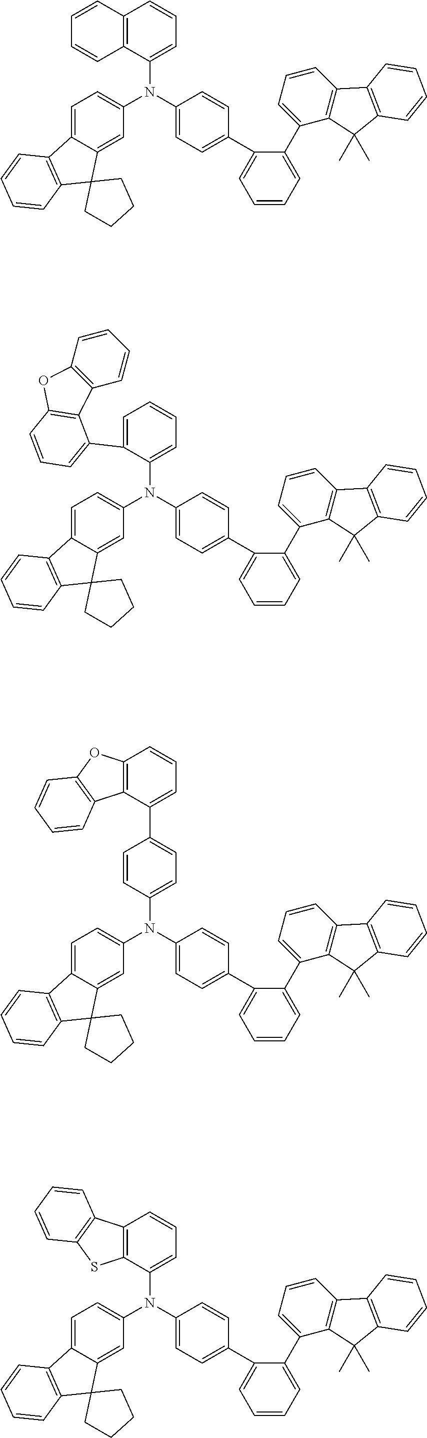 [SMILES: CC1(C)c2ccccc2-c2cccc(-c3ccccc3-c3ccc(N(c4ccc5c(c4)C4(CCCC4)c4ccccc4-5)c4cccc5ccccc45)cc3)c21 ; CC1(C)c2ccccc2-c2cccc(-c3ccccc3-c3ccc(N(c4ccc5c(c4)C4(CCCC4)c4ccccc4-5)c4ccccc4-c4cccc5oc6ccccc6c45)cc3)c21 ; CC1(C)c2ccccc2-c2cccc(-c3ccccc3-c3ccc(N(c4ccc(-c5cccc6oc7ccccc7c56)cc4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4cccc5c4sc4ccccc45)cc3)c21]

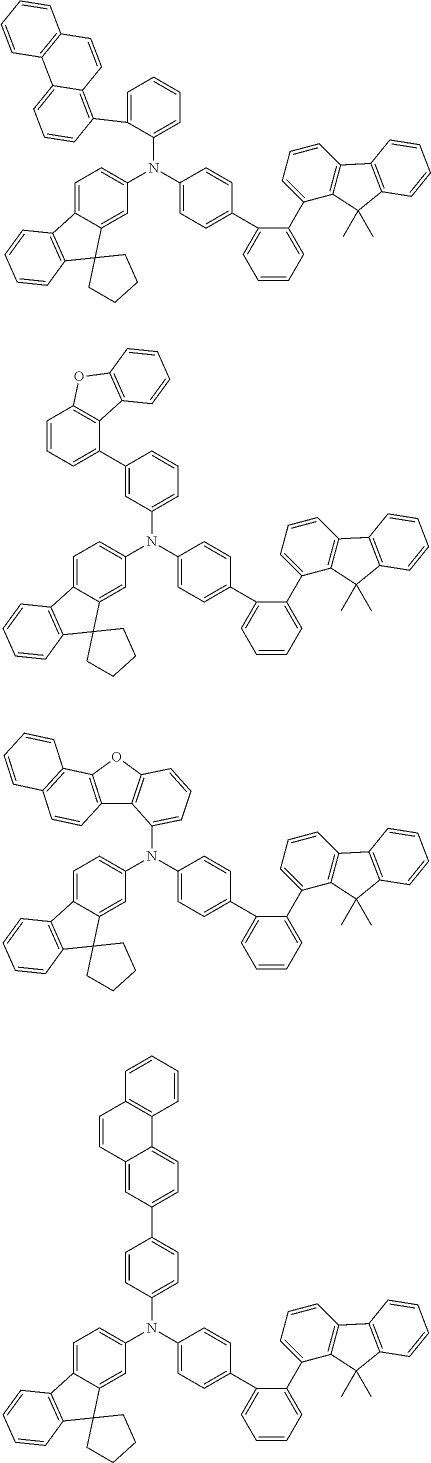 [SMILES: CC1(C)c2ccccc2-c2cccc(-c3ccccc3-c3ccc(N(c4ccc5c(c4)C4(CCCC4)c4ccccc4-5)c4ccccc4-c4cccc5c4ccc4ccccc45)cc3)c21 ; CC1(C)c2ccccc2-c2cccc(-c3ccccc3-c3ccc(N(c4cccc(-c5cccc6oc7ccccc7c56)c4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4cccc5oc6c7ccccc7ccc6c45)cc3)c21 ; CC1(C)c2ccccc2-c2cccc(-c3ccccc3-c3ccc(N(c4ccc(-c5ccc6c(ccc7ccccc76)c5)cc4)c4ccc5c(c4)C4(CCCC4)c4ccccc4-5)cc3)c21]

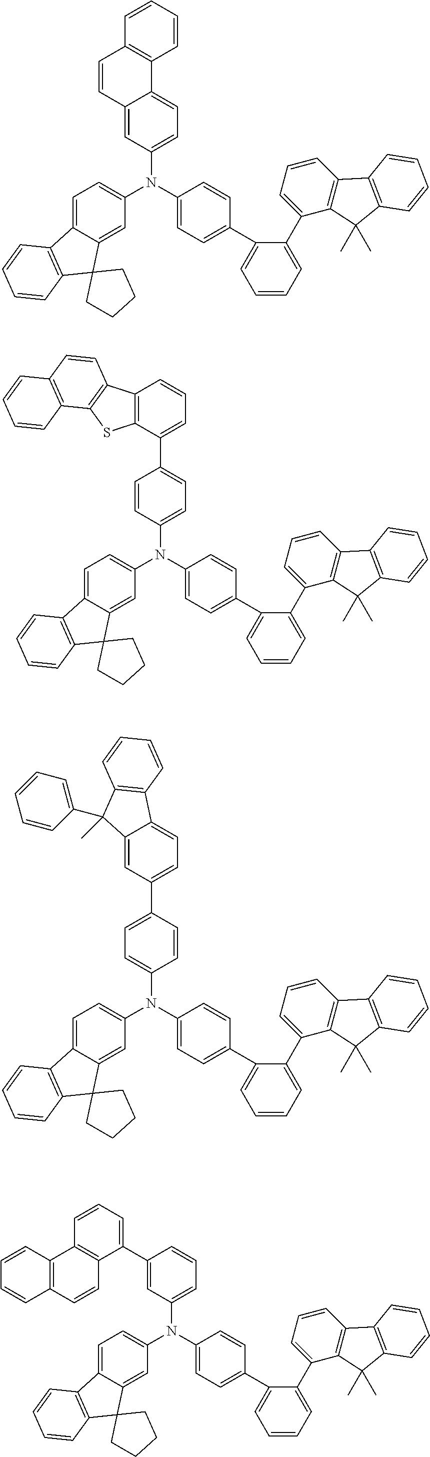 [SMILES: CC1(C)c2ccccc2-c2cccc(-c3ccccc3-c3ccc(N(c4ccc5c(c4)C4(CCCC4)c4ccccc4-5)c4ccc5c(ccc6ccccc65)c4)cc3)c21 ; CC1(C)c2ccccc2-c2cccc(-c3ccccc3-c3ccc(N(c4ccc(-c5cccc6c5sc5c7ccccc7ccc65)cc4)c4ccc5c(c4)C4(CCCC4)c4ccccc4-5)cc3)c21 ; CC1(C)c2ccccc2-c2cccc(-c3ccccc3-c3ccc(N(c4ccc(-c5ccc6c(c5)C(C)(c5ccccc5)c5ccccc5-6)cc4)c4ccc5c(c4)C4(CCCC4)c4ccccc4-5)cc3)c21 ; CC1(C)c2ccccc2-c2cccc(-c3ccccc3-c3ccc(N(c4cccc(-c5cccc6c5ccc5ccccc56)c4)c4ccc5c(c4)C4(CCCC4)c4ccccc4-5)cc3)c21]

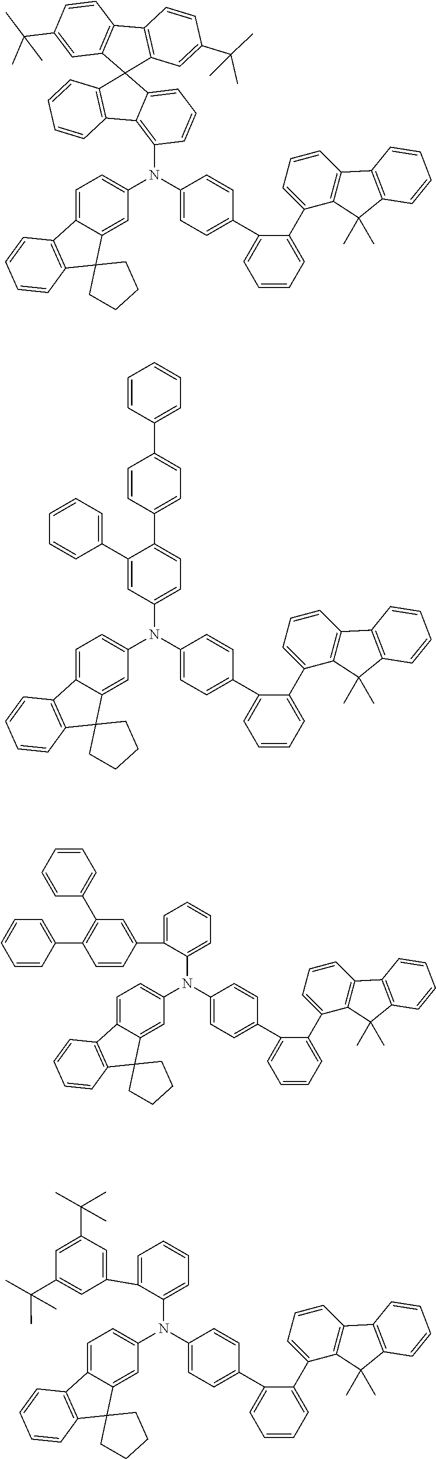

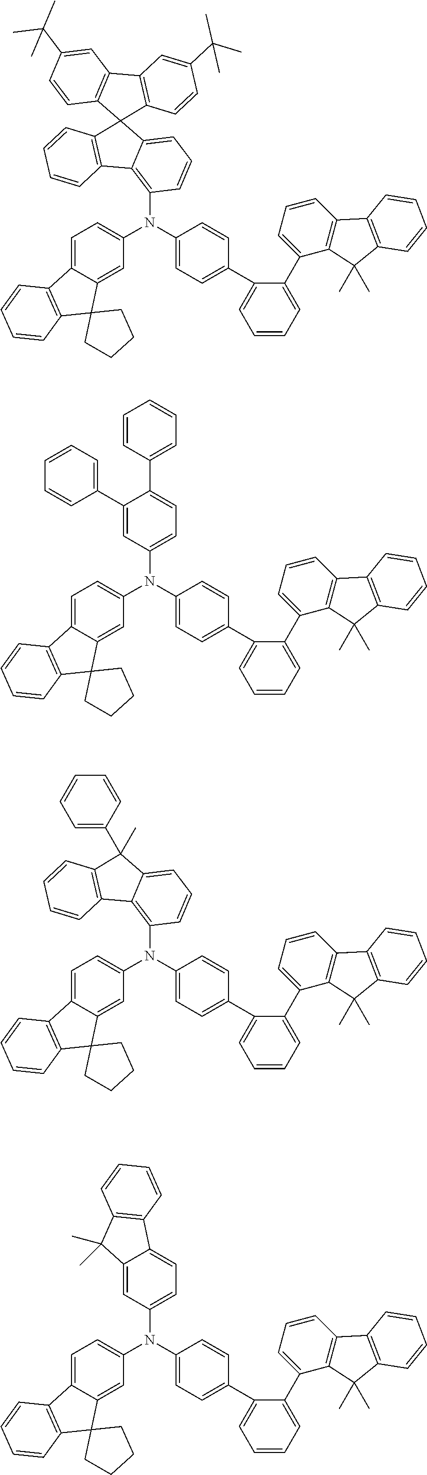 [SMILES: CC(C)(C)c1ccc2c(c1)-c1cc(C(C)(C)C)ccc1C21c2ccccc2-c2c(N(c3ccc(-c4ccccc4-c4cccc5c4C(C)(C)c4ccccc4-5)cc3)c3ccc4c(c3)C3(CCCC3)c3ccccc3-4)cccc21 ; CC1(C)c2ccccc2-c2cccc(-c3ccccc3-c3ccc(N(c4ccc(-c5ccccc5)c(-c5ccccc5)c4)c4ccc5c(c4)C4(CCCC4)c4ccccc4-5)cc3)c21 ; CC1(C)c2ccccc2-c2cccc(-c3ccccc3-c3ccc(N(c4ccc5c(c4)C4(CCCC4)c4ccccc4-5)c4cccc5c4-c4ccccc4C5(C)c4ccccc4)cc3)c21 ; CC1(C)c2ccccc2-c2ccc(N(c3ccc(-c4ccccc4-c4cccc5c4C(C)(C)c4ccccc4-5)cc3)c3ccc4c(c3)C3(CCCC3)c3ccccc3-4)cc21]

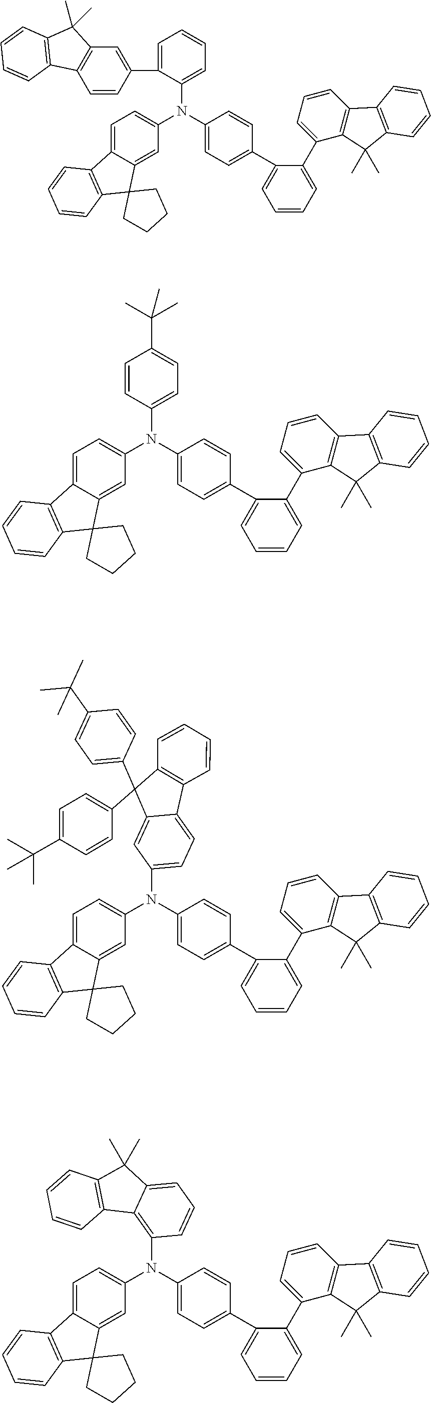 [SMILES: CC1(C)c2ccccc2-c2ccc(-c3ccccc3N(c3ccc(-c4ccccc4-c4cccc5c4C(C)(C)c4ccccc4-5)cc3)c3ccc4c(c3)C3(CCCC3)c3ccccc3-4)cc21 ; CC(C)(C)c1ccc(N(c2ccc(-c3ccccc3-c3cccc4c3C(C)(C)c3ccccc3-4)cc2)c2ccc3c(c2)C2(CCCC2)c2ccccc2-3)cc1 ; CC(C)(C)c1ccc(C2(c3ccc(C(C)(C)C)cc3)c3ccccc3-c3ccc(N(c4ccc(-c5ccccc5-c5cccc6c5C(C)(C)c5ccccc5-6)cc4)c4ccc5c(c4)C4(CCCC4)c4ccccc4-5)cc32)cc1 ; CC1(C)c2ccccc2-c2c(N(c3ccc(-c4ccccc4-c4cccc5c4C(C)(C)c4ccccc4-5)cc3)c3ccc4c(c3)C3(CCCC3)c3ccccc3-4)cccc21]

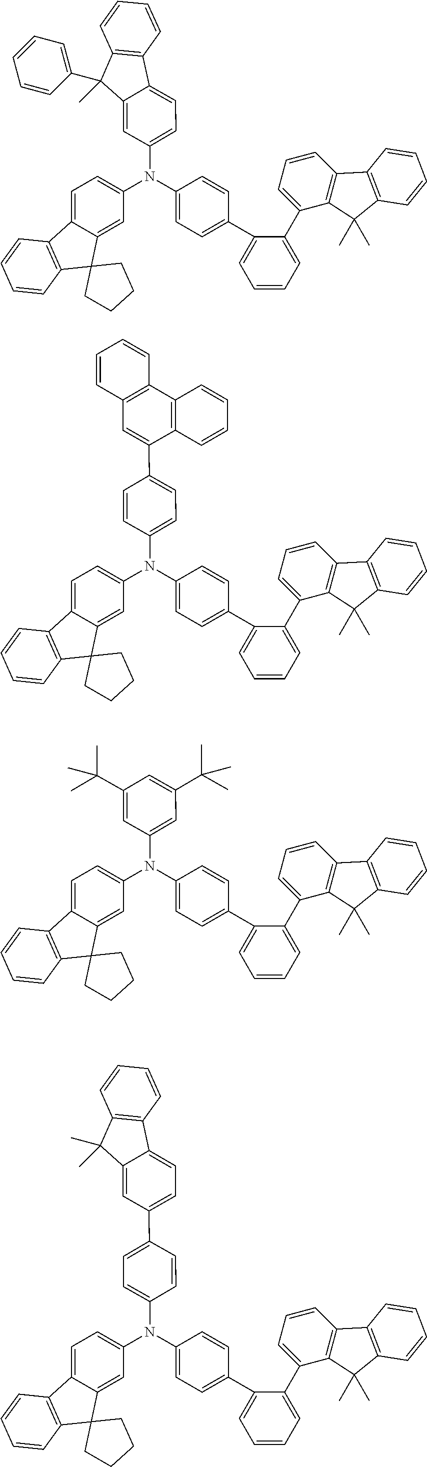

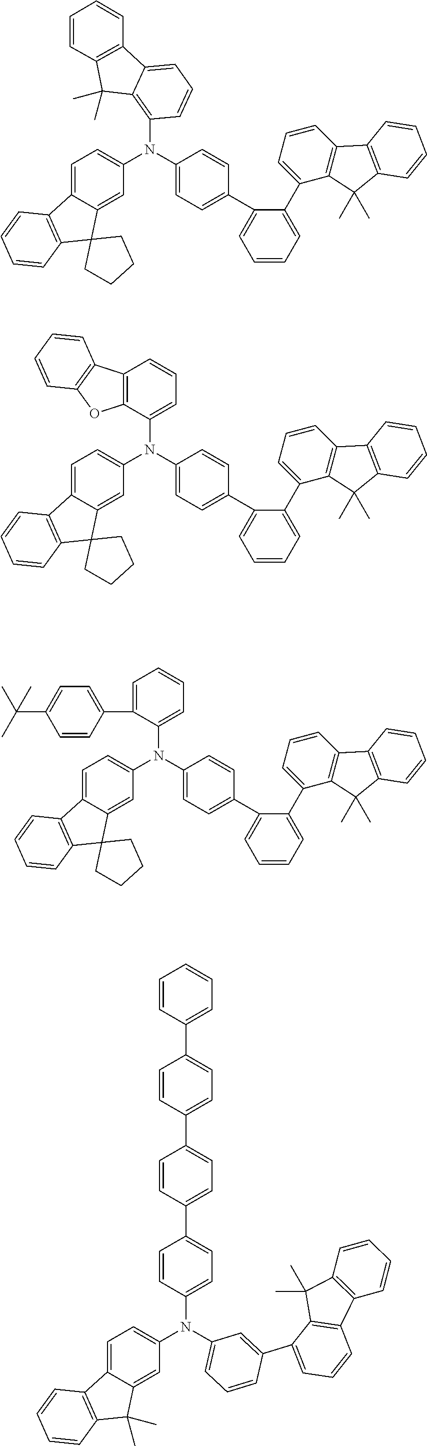 [SMILES: CC1(C)c2ccccc2-c2cccc(-c3ccccc3-c3ccc(N(c4ccc5c(c4)C4(CCCC4)c4ccccc4-5)c4cccc5c4C(C)(C)c4ccccc4-5)cc3)c21 ; CC1(C)c2ccccc2-c2cccc(-c3ccccc3-c3ccc(N(c4ccc5c(c4)C4(CCCC4)c4ccccc4-5)c4cccc5c4oc4ccccc45)cc3)c21 ; CC(C)(C)c1ccc(-c2ccccc2N(c2ccc(-c3ccccc3-c3cccc4c3C(C)(C)c3ccccc3-4)cc2)c2ccc3c(c2)C2(CCCC2)c2ccccc2-3)cc1 ; CC1(C)c2ccccc2-c2ccc(N(c3ccc(-c4ccc(-c5ccc(-c6ccccc6)cc5)cc4)cc3)c3cccc(-c4cccc5c4C(C)(C)c4ccccc4-5)c3)cc21]

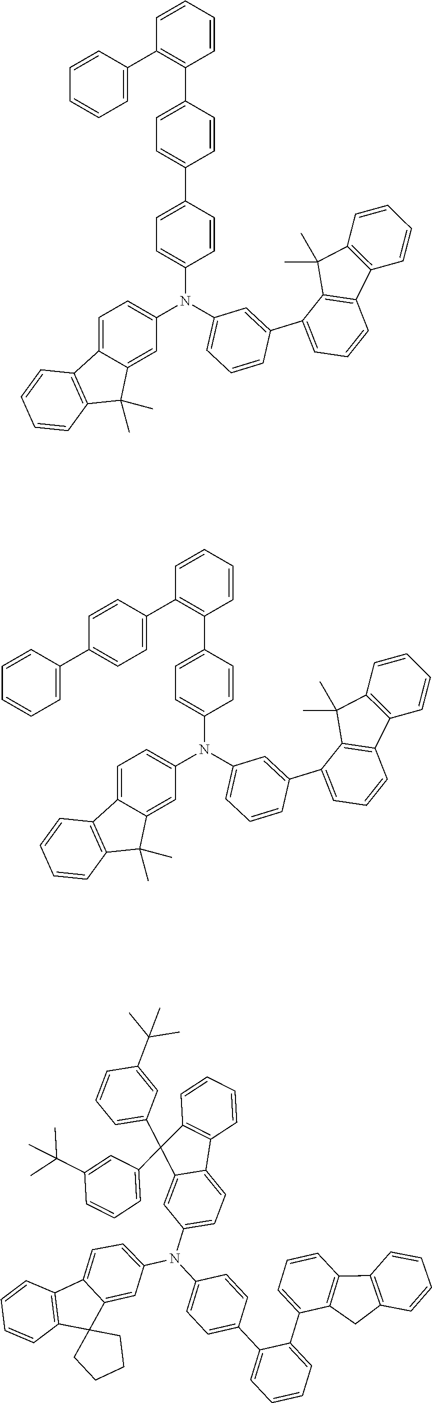 [SMILES: CC1(C)c2ccccc2-c2ccc(N(c3ccc(-c4ccc(-c5ccccc5-c5ccccc5)cc4)cc3)c3cccc(-c4cccc5c4C(C)(C)c4ccccc4-5)c3)cc21 ; CC1(C)c2ccccc2-c2ccc(N(c3ccc(-c4ccccc4-c4ccc(-c5ccccc5)cc4)cc3)c3cccc(-c4cccc5c4C(C)(C)c4ccccc4-5)c3)cc21 ; CC(C)(C)c1cccc(C2(c3cccc(C(C)(C)C)c3)c3ccccc3-c3ccc(N(c4ccc(-c5ccccc5-c5cccc6c5Cc5ccccc5-6)cc4)c4ccc5c(c4)C4(CCCC4)c4ccccc4-5)cc32)c1]

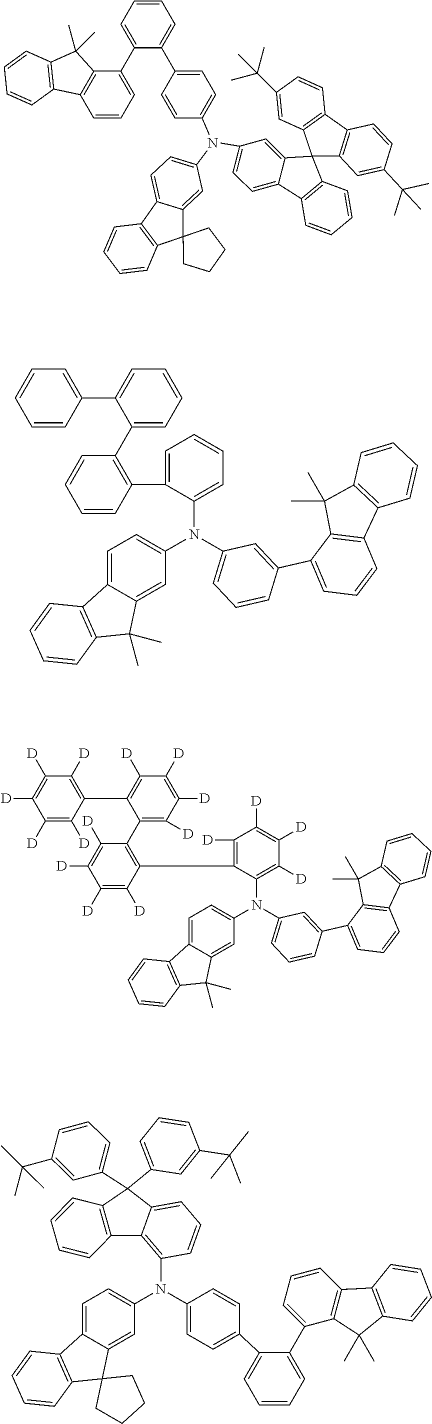

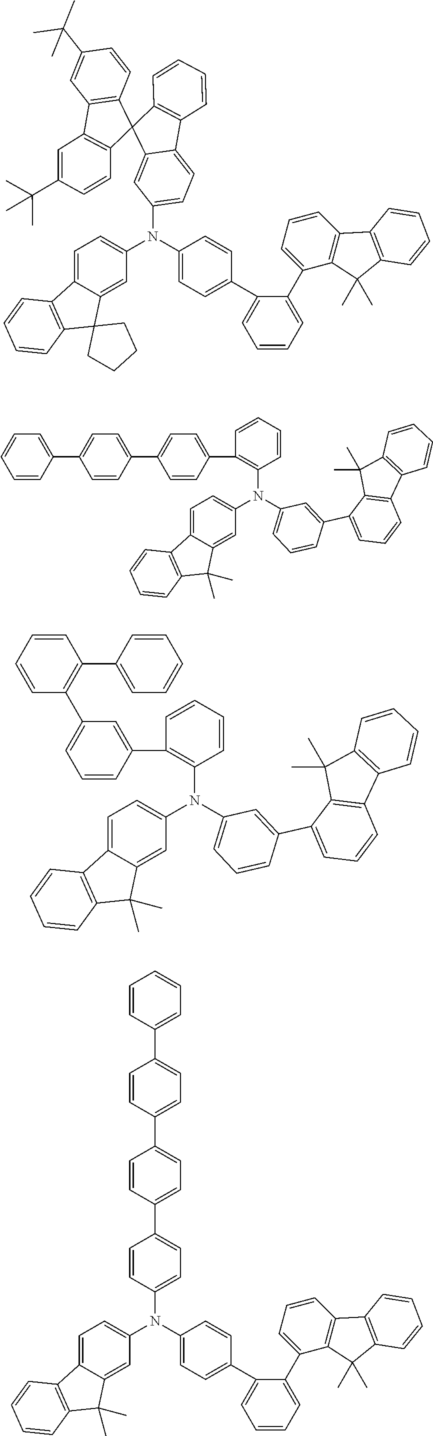

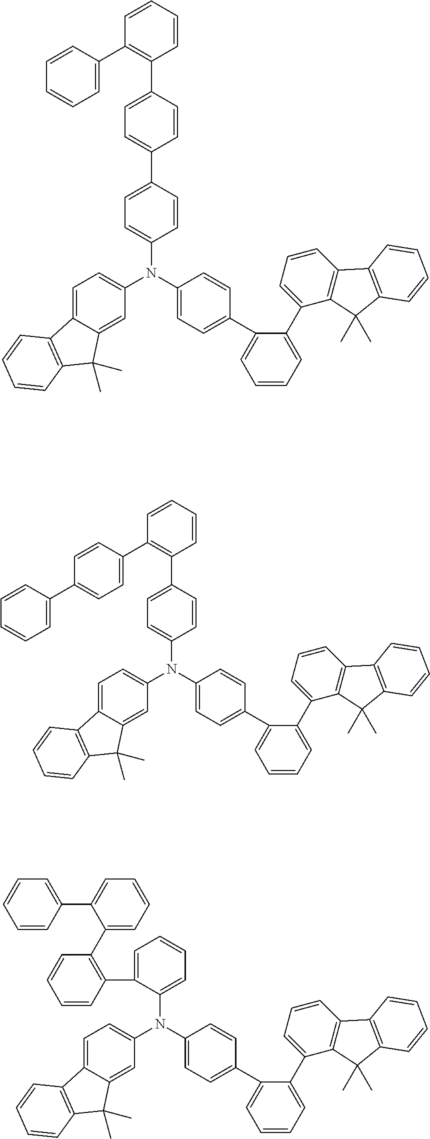 [SMILES: CC1(C)c2ccccc2-c2ccc(N(c3ccc(-c4ccc(-c5ccccc5-c5ccccc5)cc4)cc3)c3ccc(-c4ccccc4-c4cccc5c4C(C)(C)c4ccccc4-5)cc3)cc21 ; CC1(C)c2ccccc2-c2ccc(N(c3ccc(-c4ccccc4-c4ccc(-c5ccccc5)cc4)cc3)c3ccc(-c4ccccc4-c4cccc5c4C(C)(C)c4ccccc4-5)cc3)cc21 ; CC1(C)c2ccccc2-c2ccc(N(c3ccc(-c4ccccc4-c4cccc5c4C(C)(C)c4ccccc4-5)cc3)c3ccccc3-c3ccccc3-c3ccccc3-c3ccccc3)cc21]

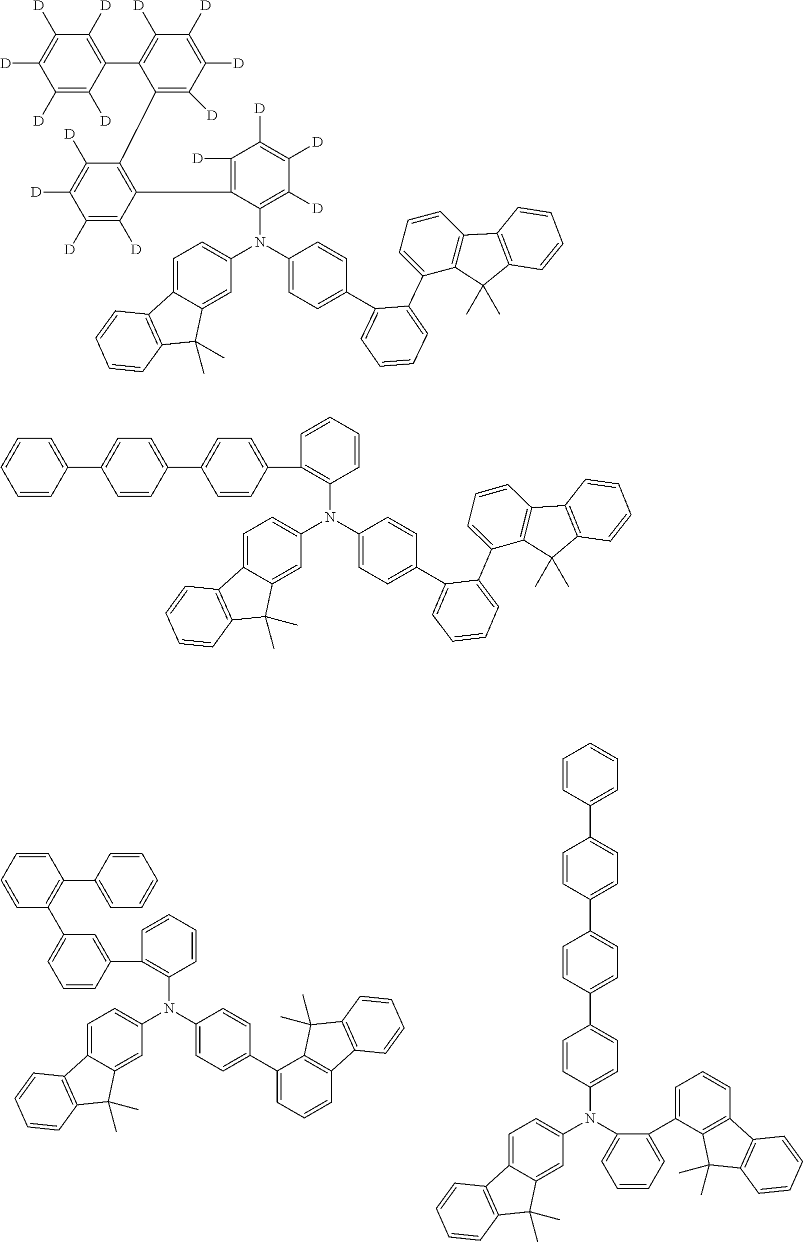

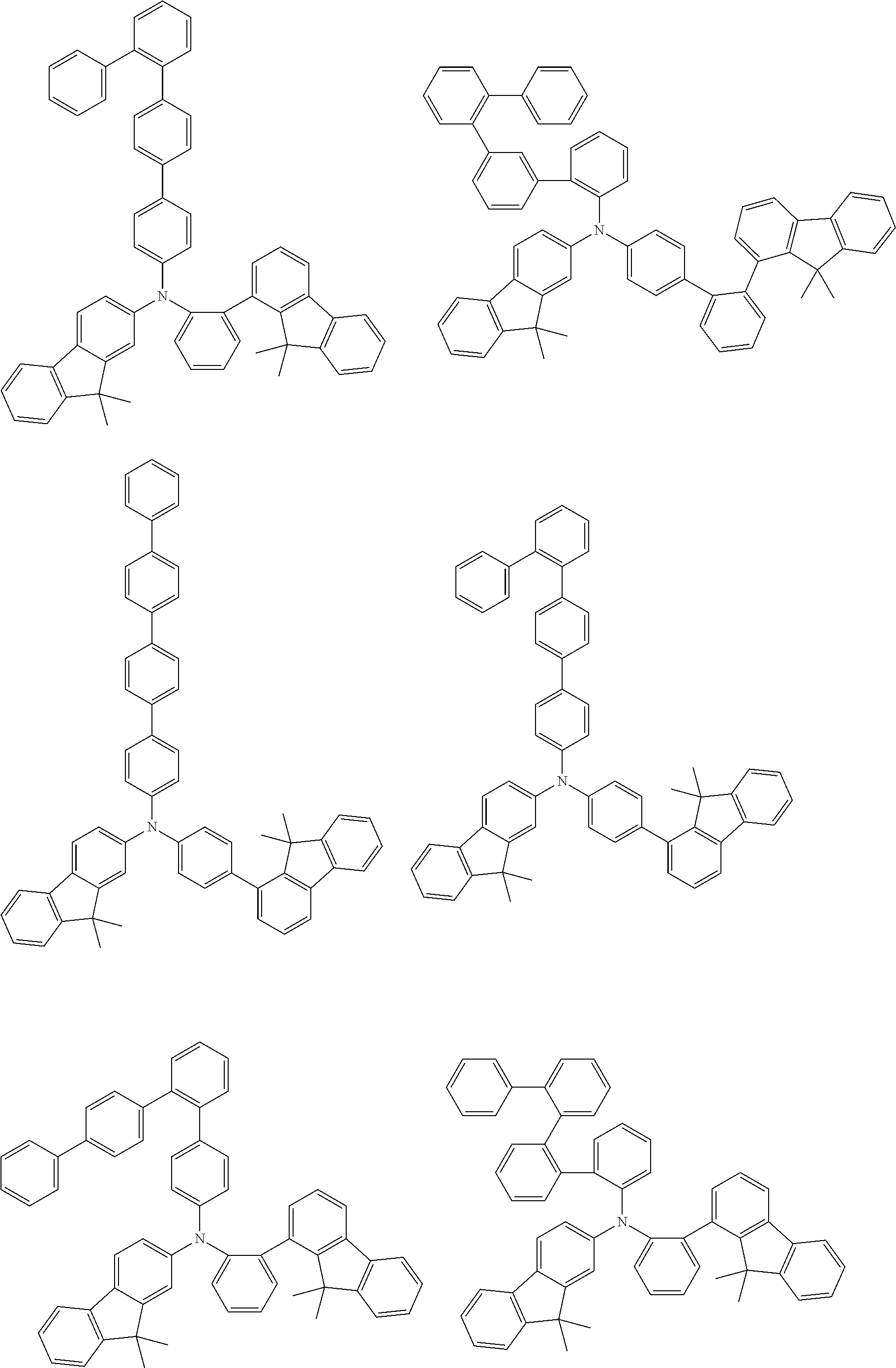

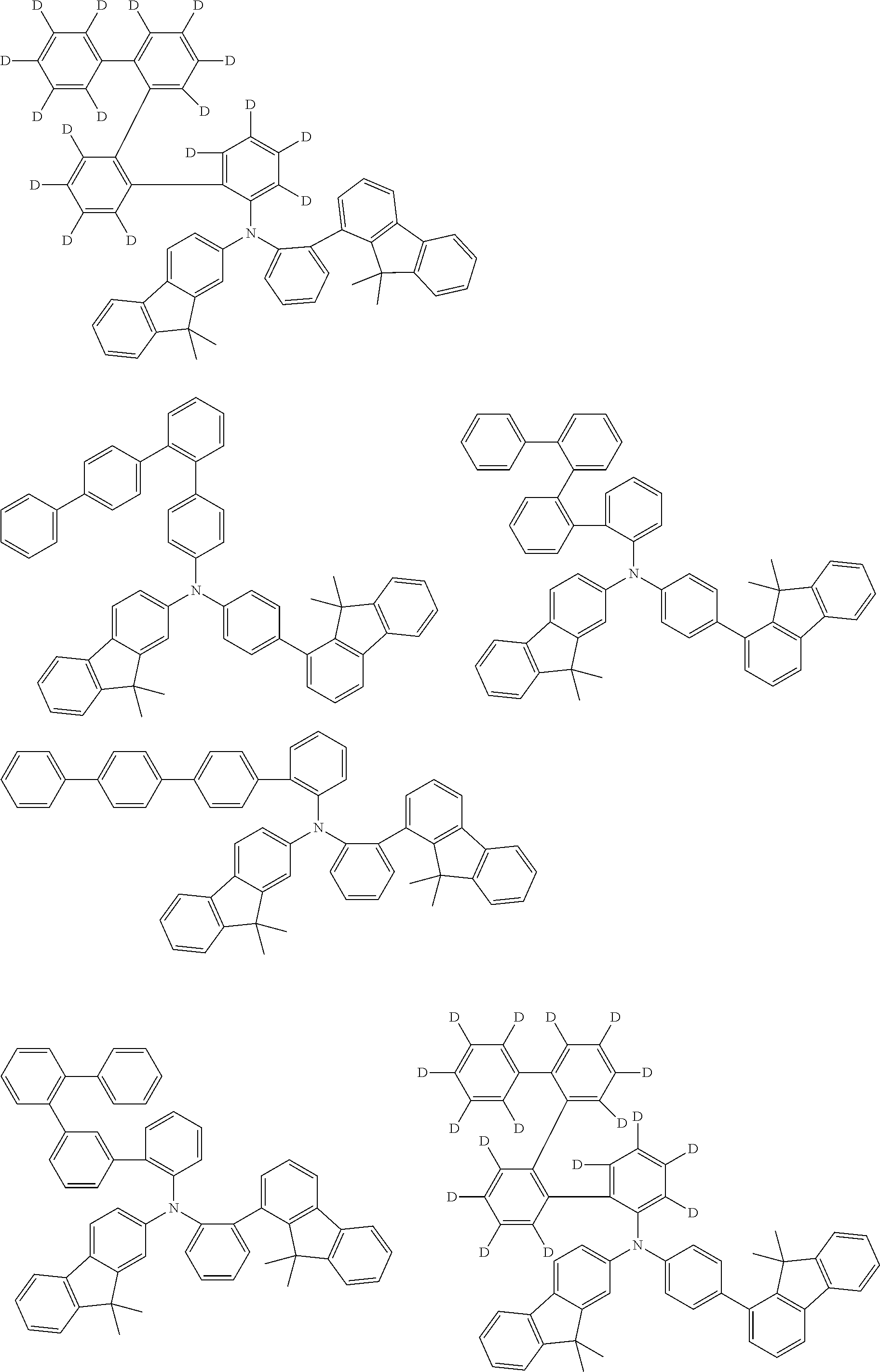

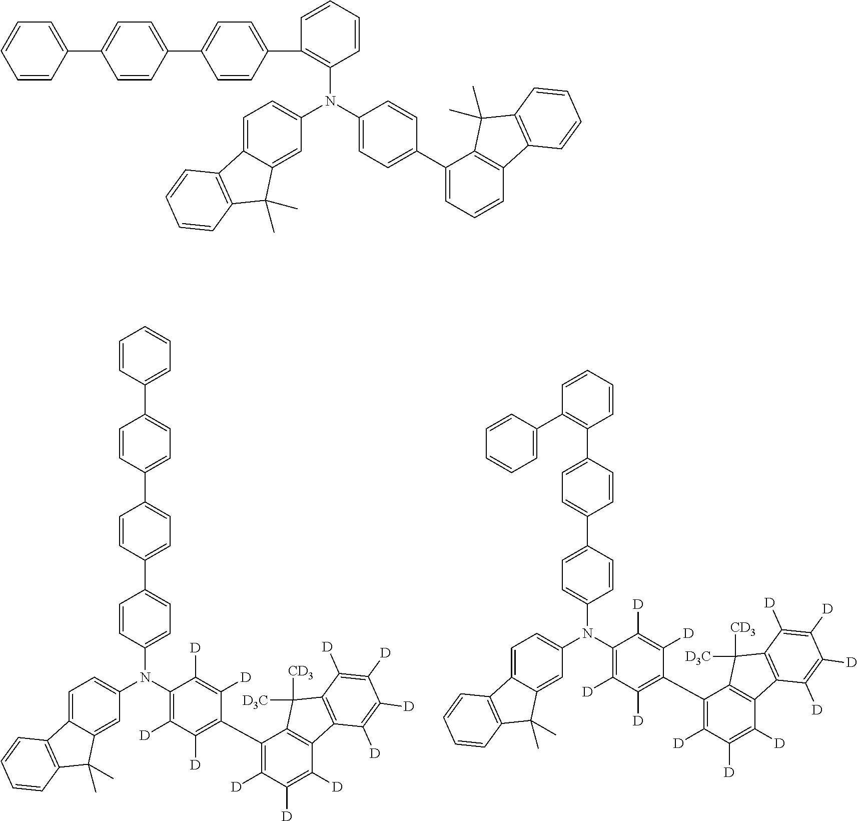

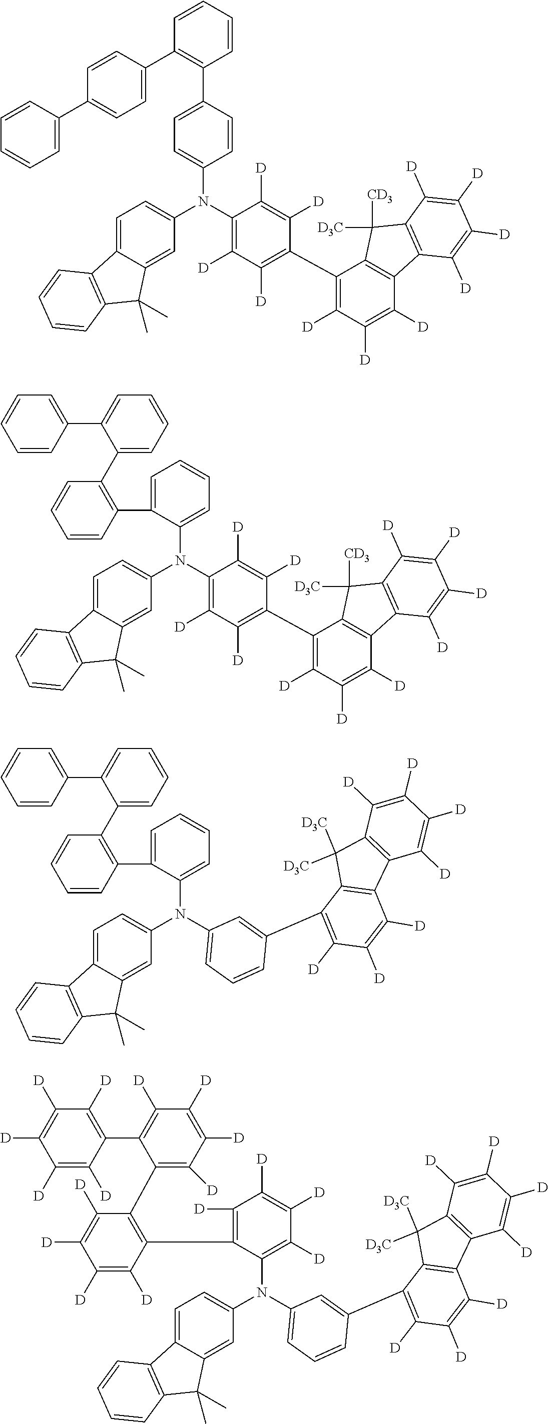

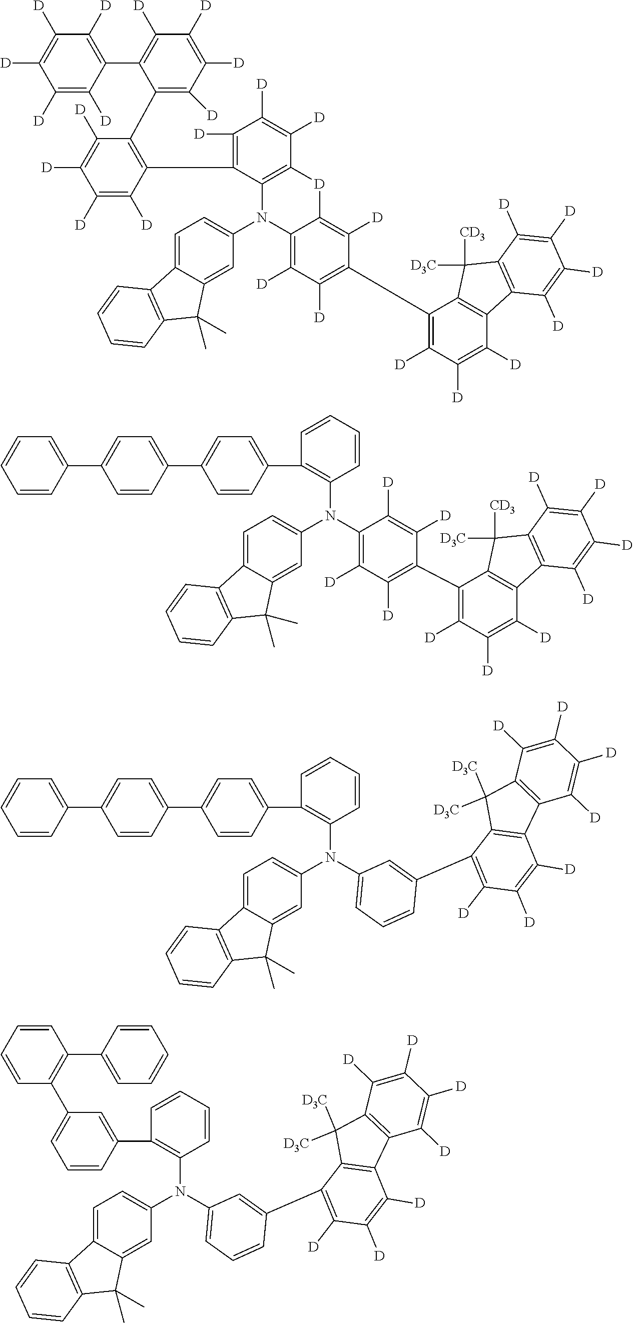

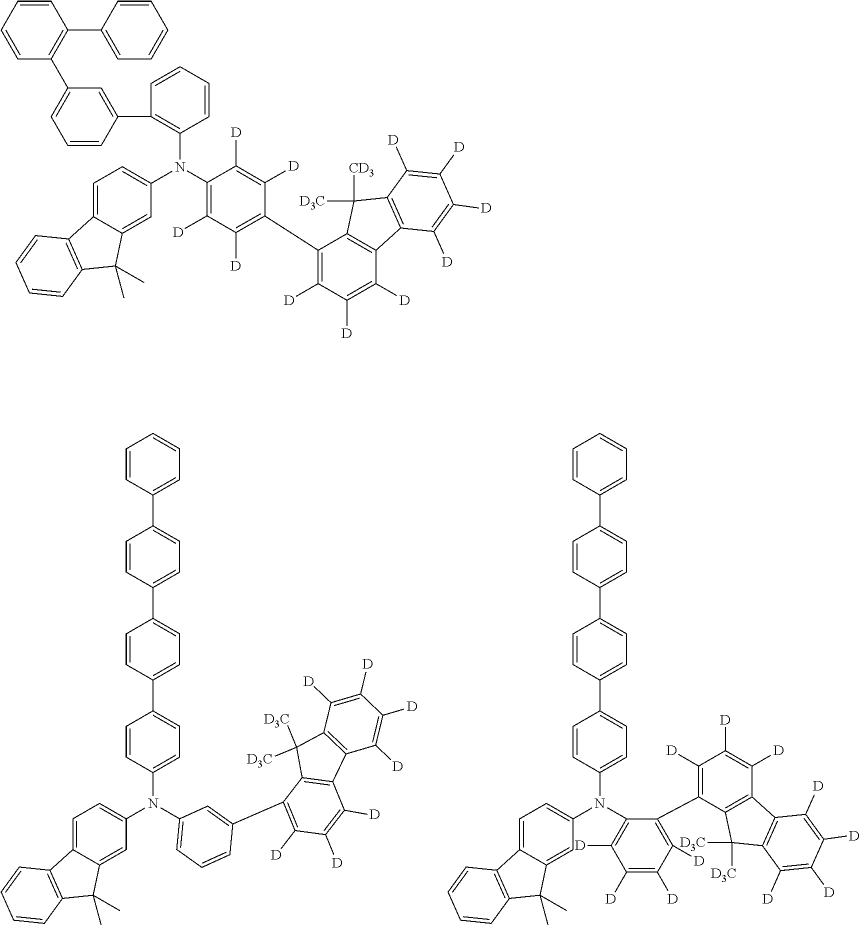

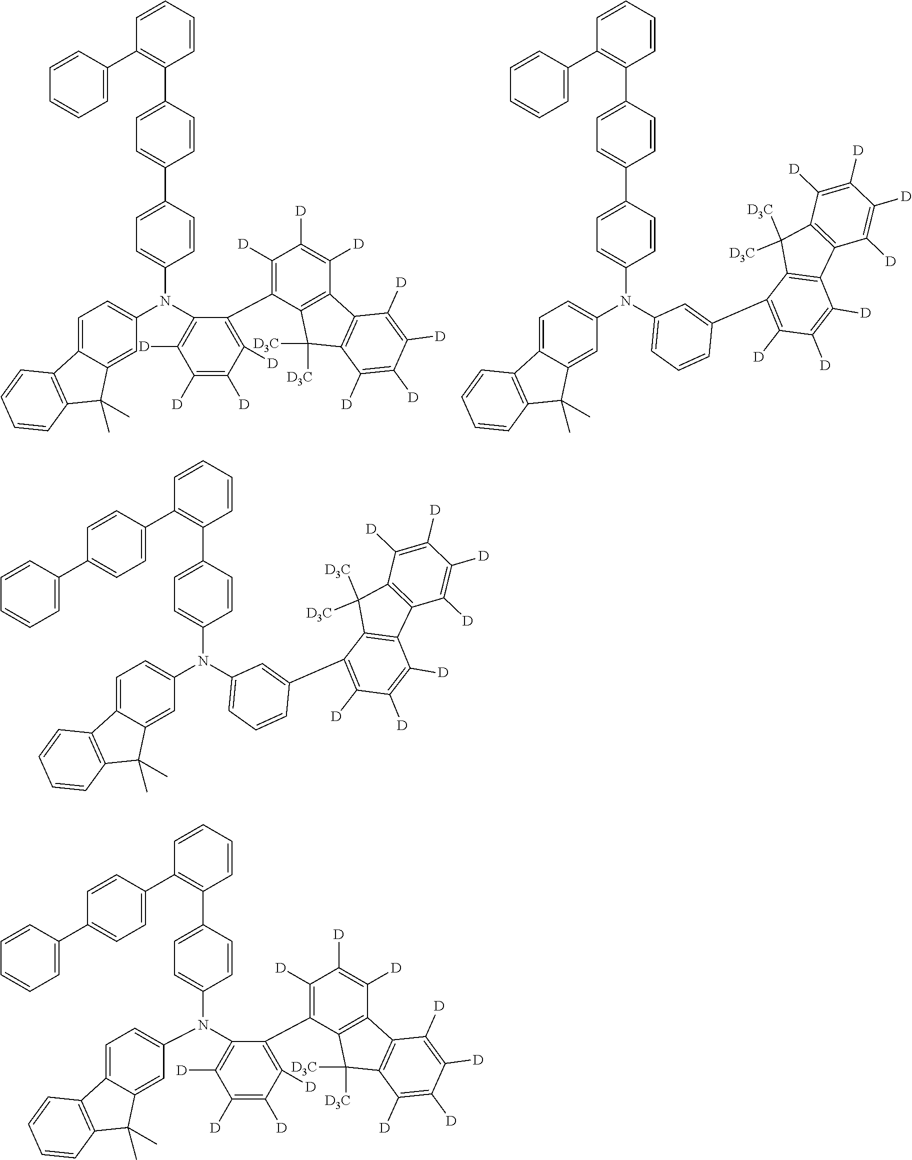

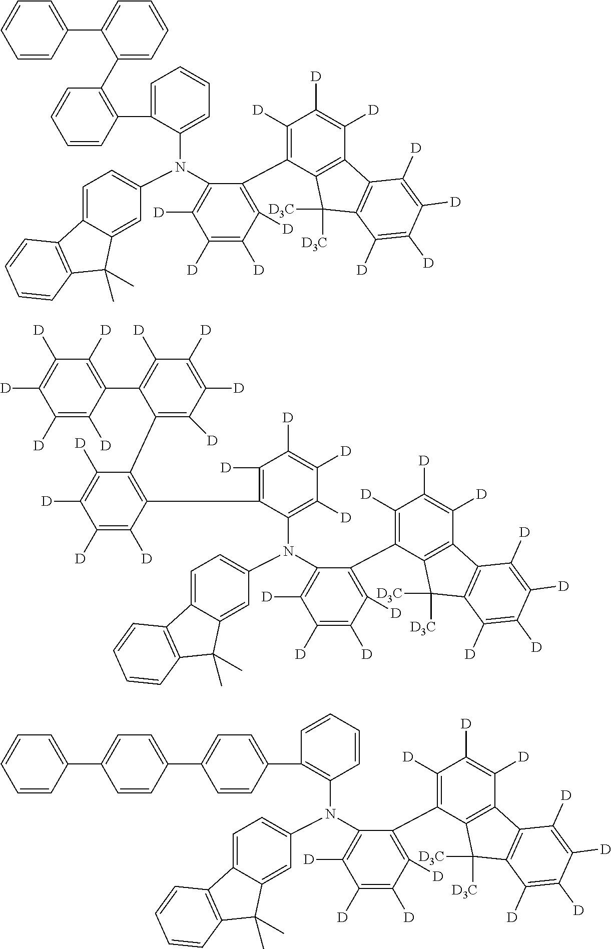

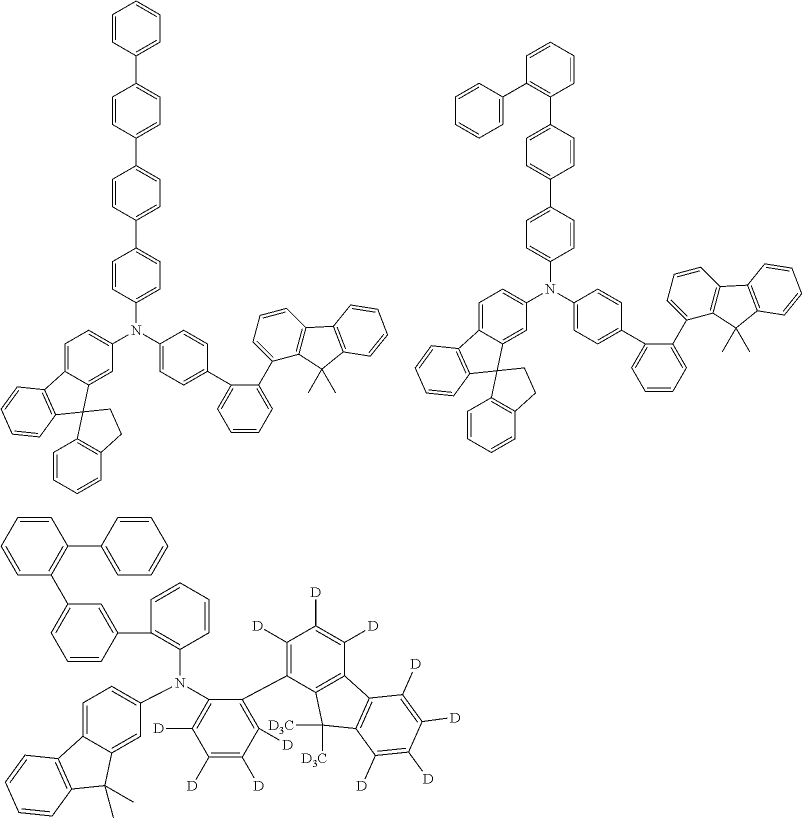

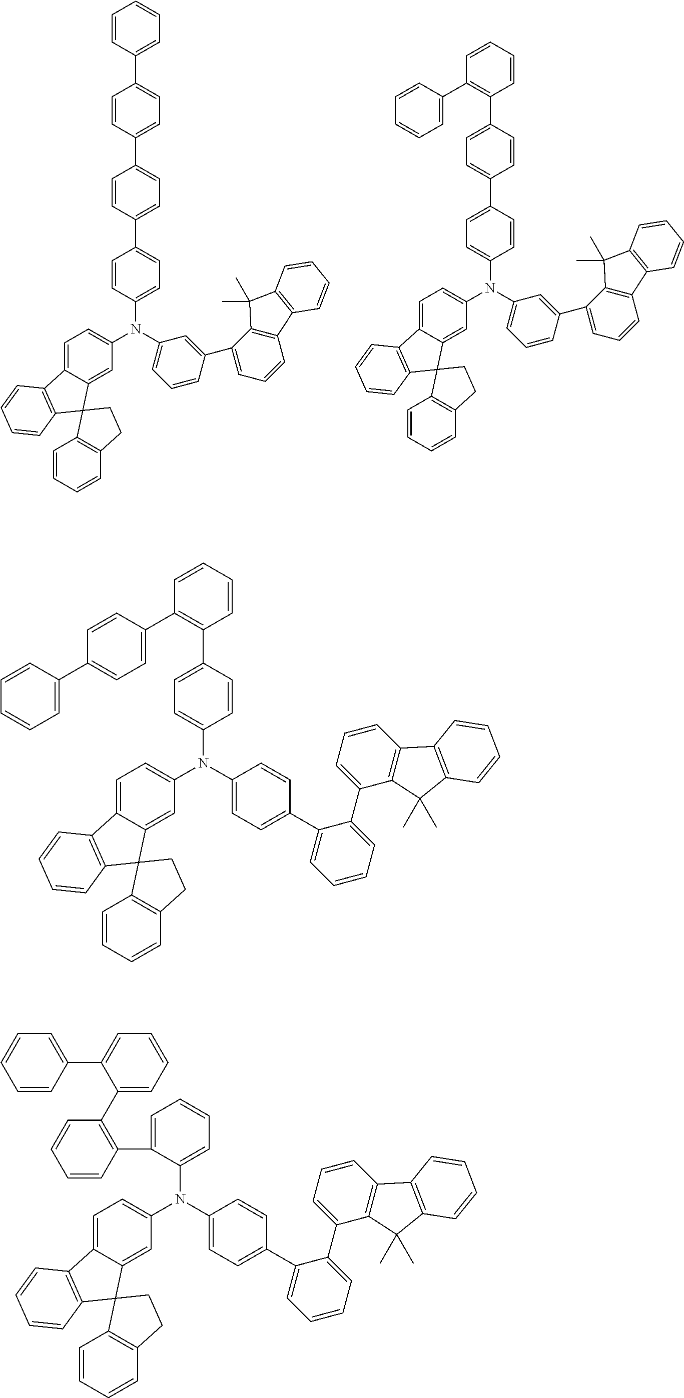 [SMILES: CC1(C)c2ccccc2-c2cccc(-c3cccc(N(c4ccc(-c5ccc(-c6ccc(-c7ccccc7)cc6)cc5)cc4)c4ccc5c(c4)C4(CCc6ccccc64)c4ccccc4-5)c3)c21 ; CC1(C)c2ccccc2-c2cccc(-c3cccc(N(c4ccc(-c5ccc(-c6ccccc6-c6ccccc6)cc5)cc4)c4ccc5c(c4)C4(CCc6ccccc64)c4ccccc4-5)c3)c21 ; CC1(C)c2ccccc2-c2cccc(-c3ccccc3-c3ccc(N(c4ccc(-c5ccccc5-c5ccc(-c6ccccc6)cc5)cc4)c4ccc5c(c4)C4(CCc6ccccc64)c4ccccc4-5)cc3)c21 ; CC1(C)c2ccccc2-c2cccc(-c3ccccc3-c3ccc(N(c4ccc5c(c4)C4(CCc6ccccc64)c4ccccc4-5)c4ccccc4-c4ccccc4-c4ccccc4-c4ccccc4)cc3)c21]

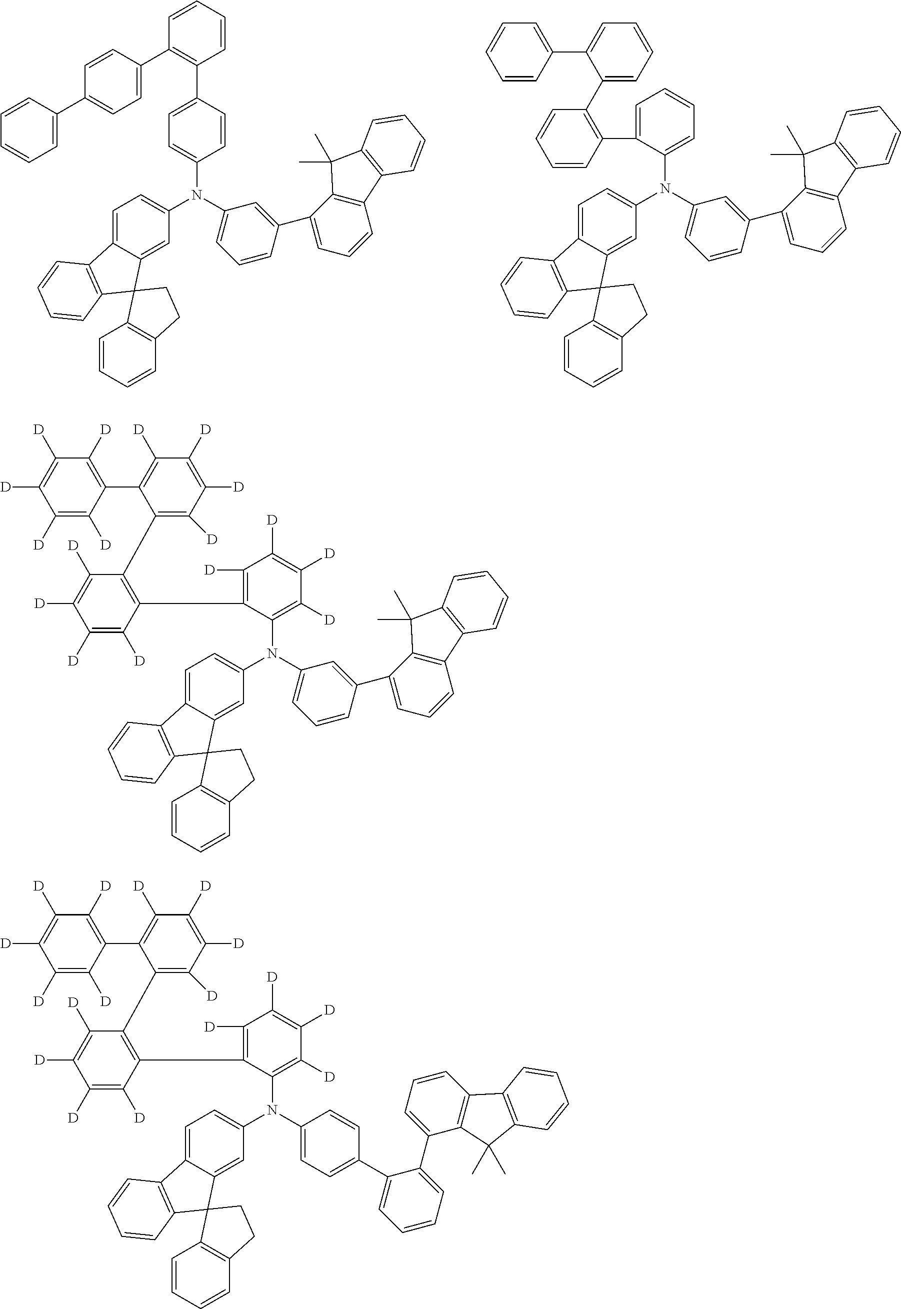

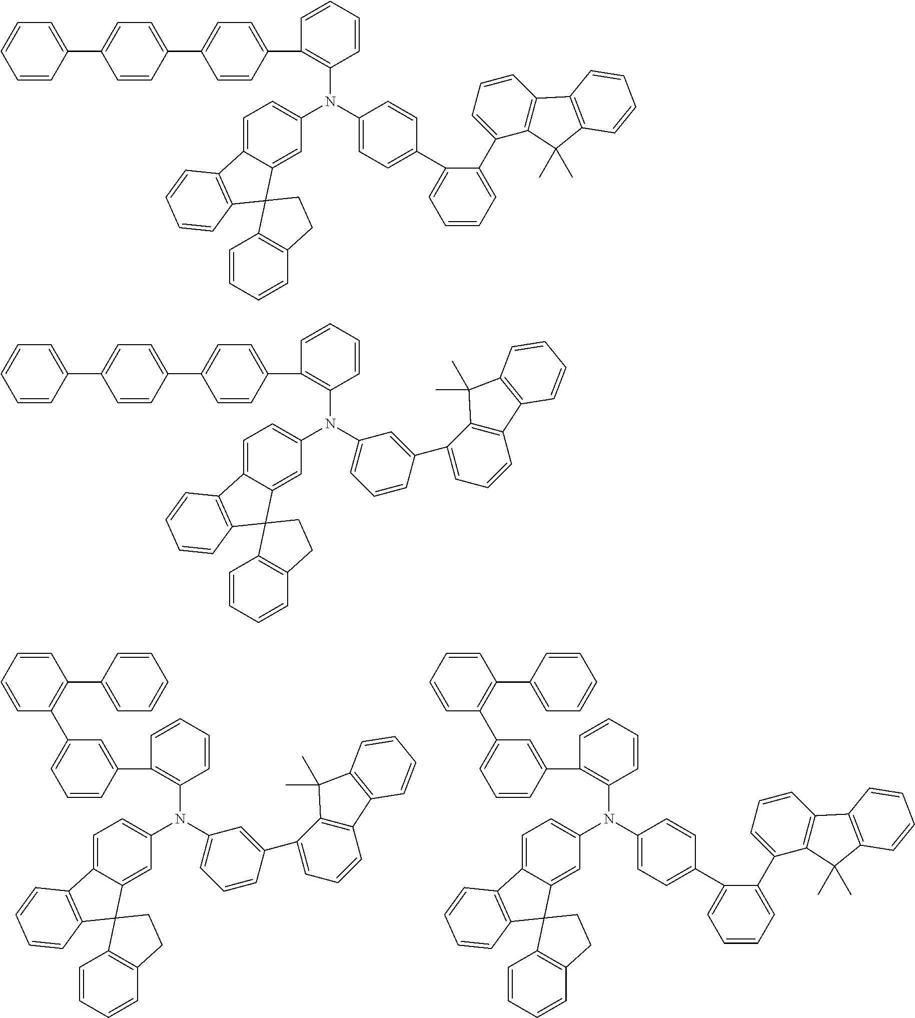

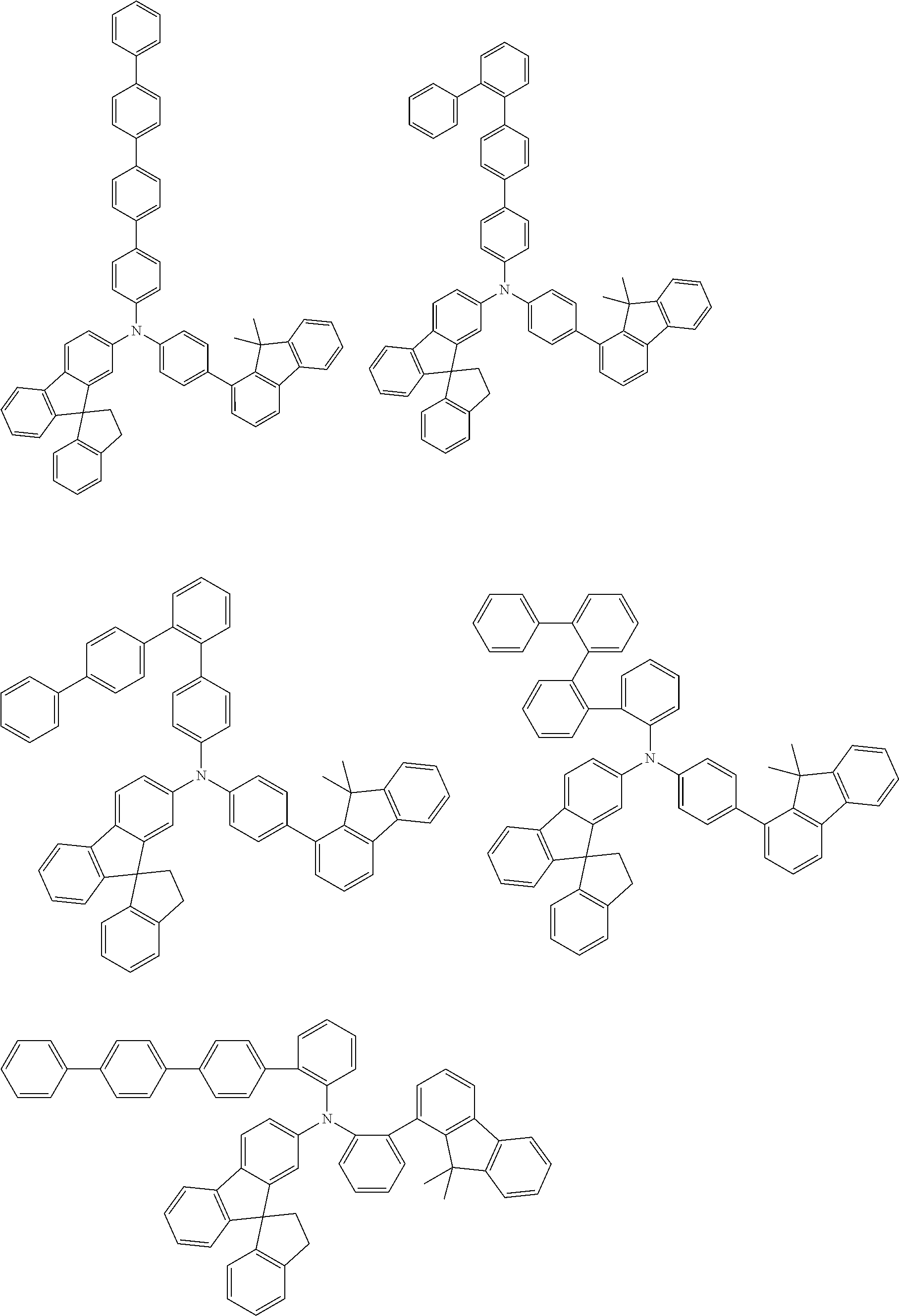

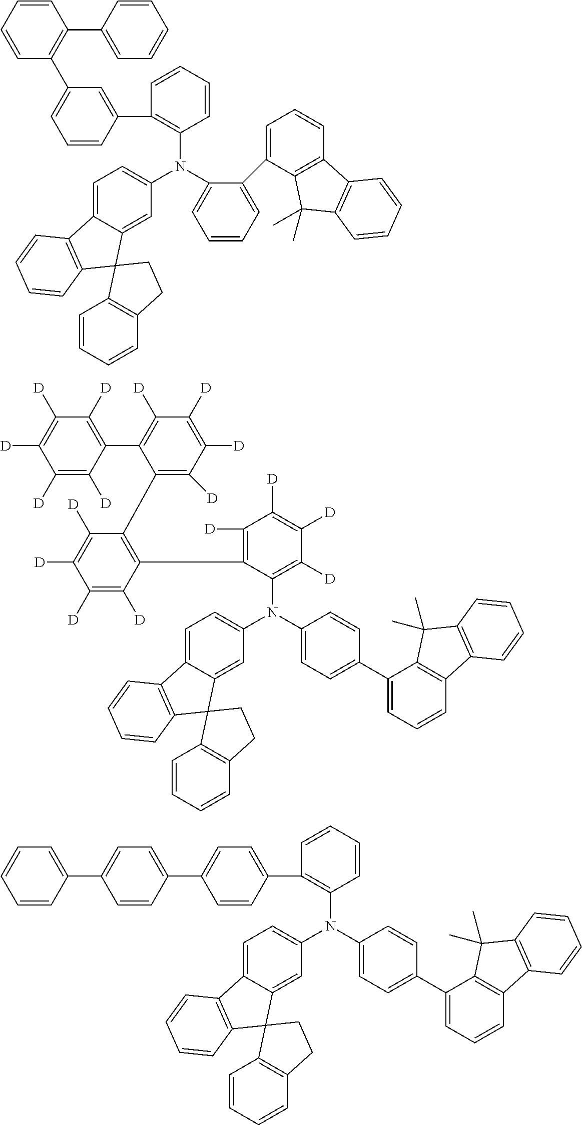

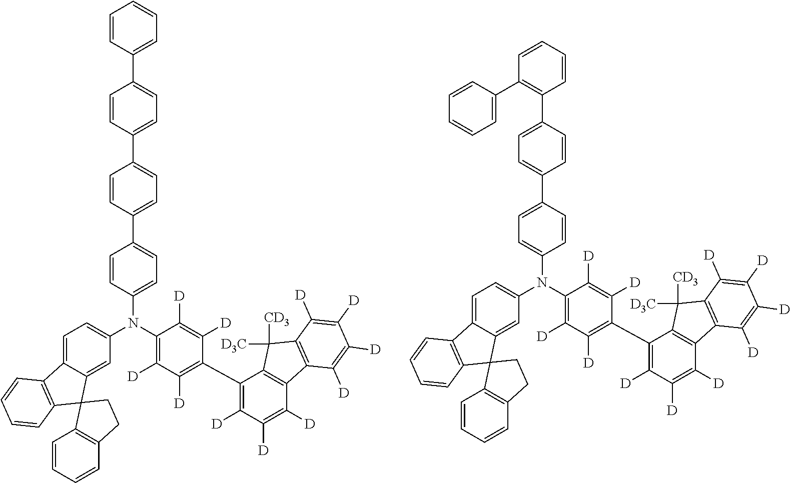

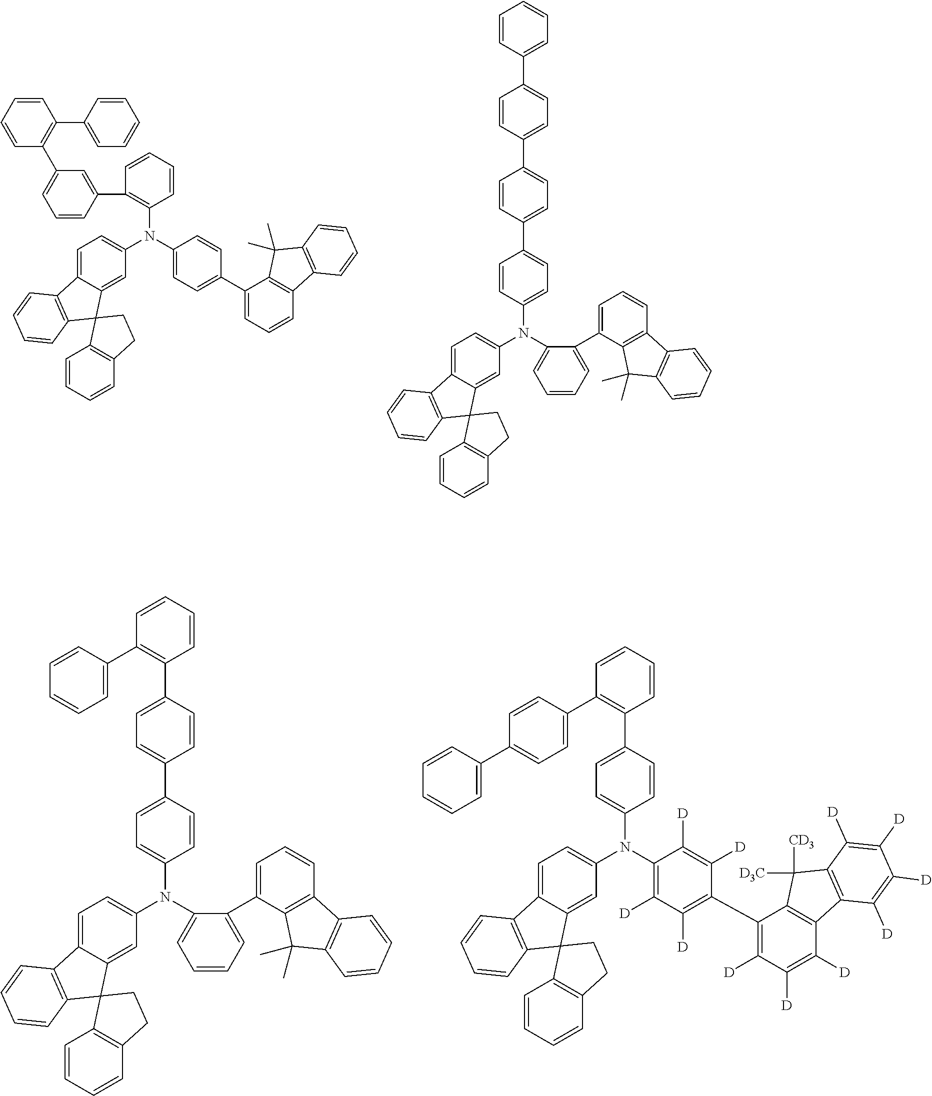

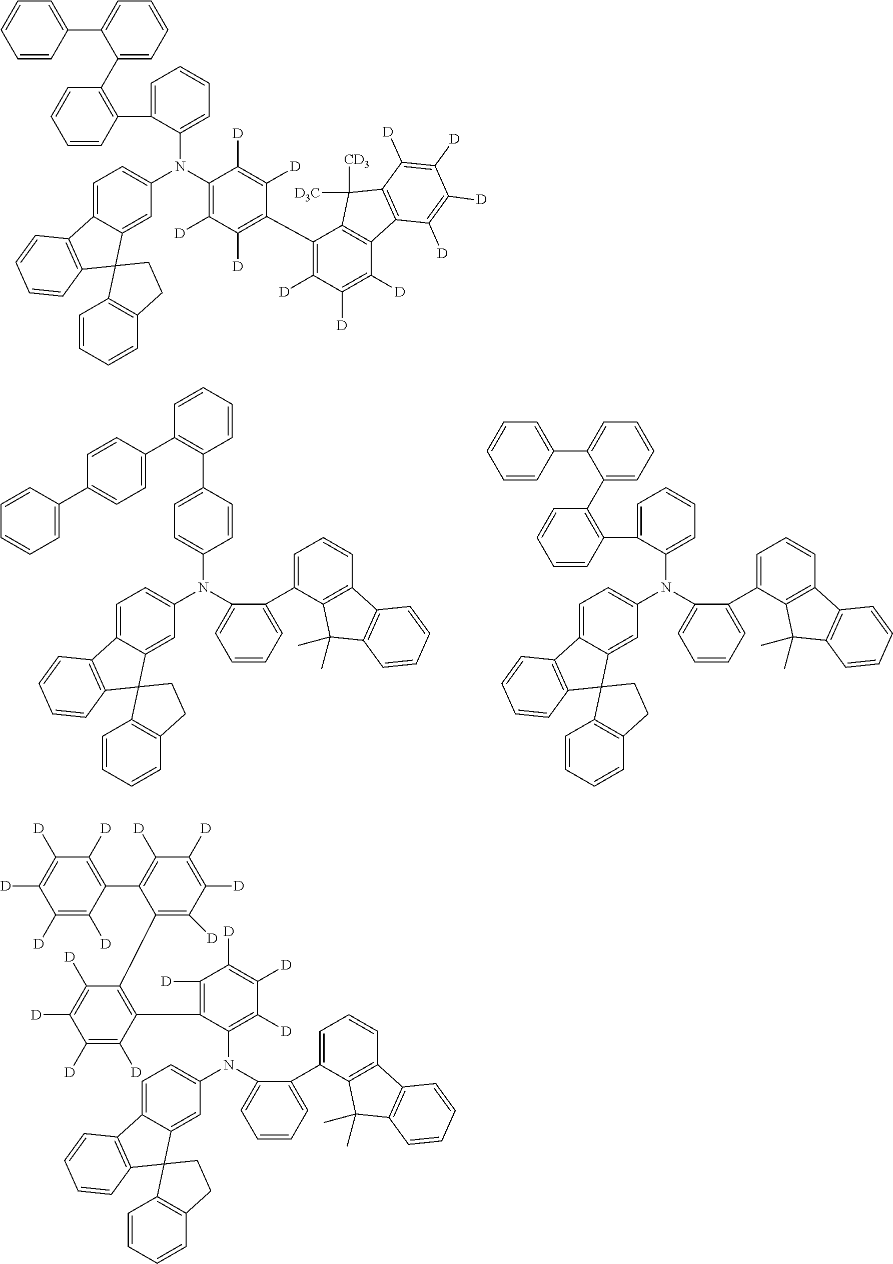

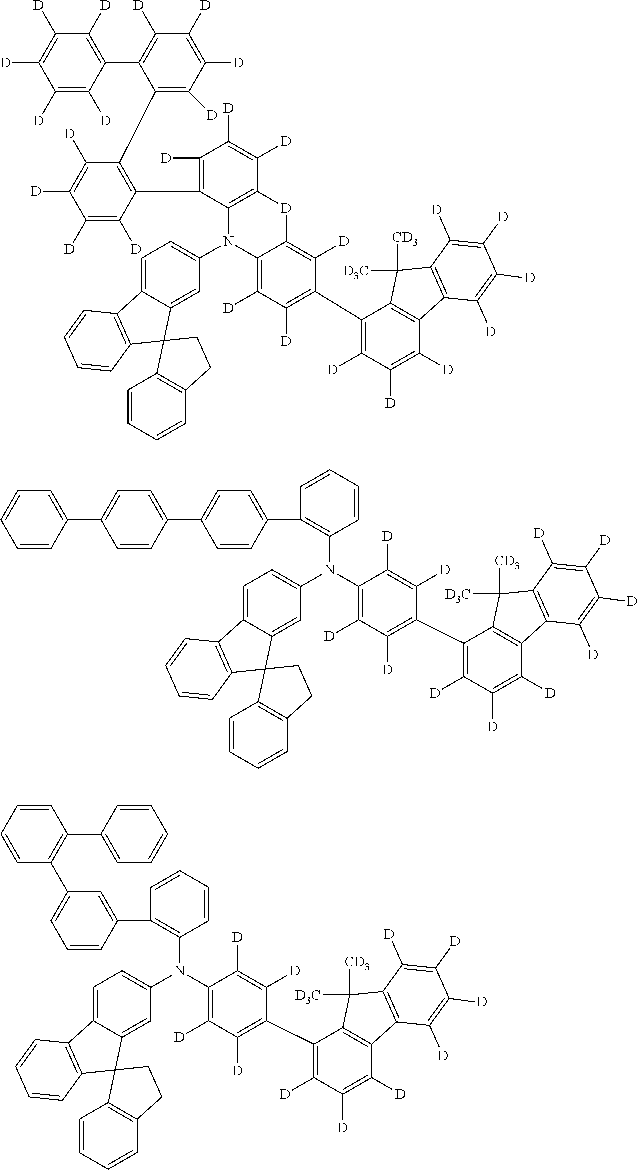

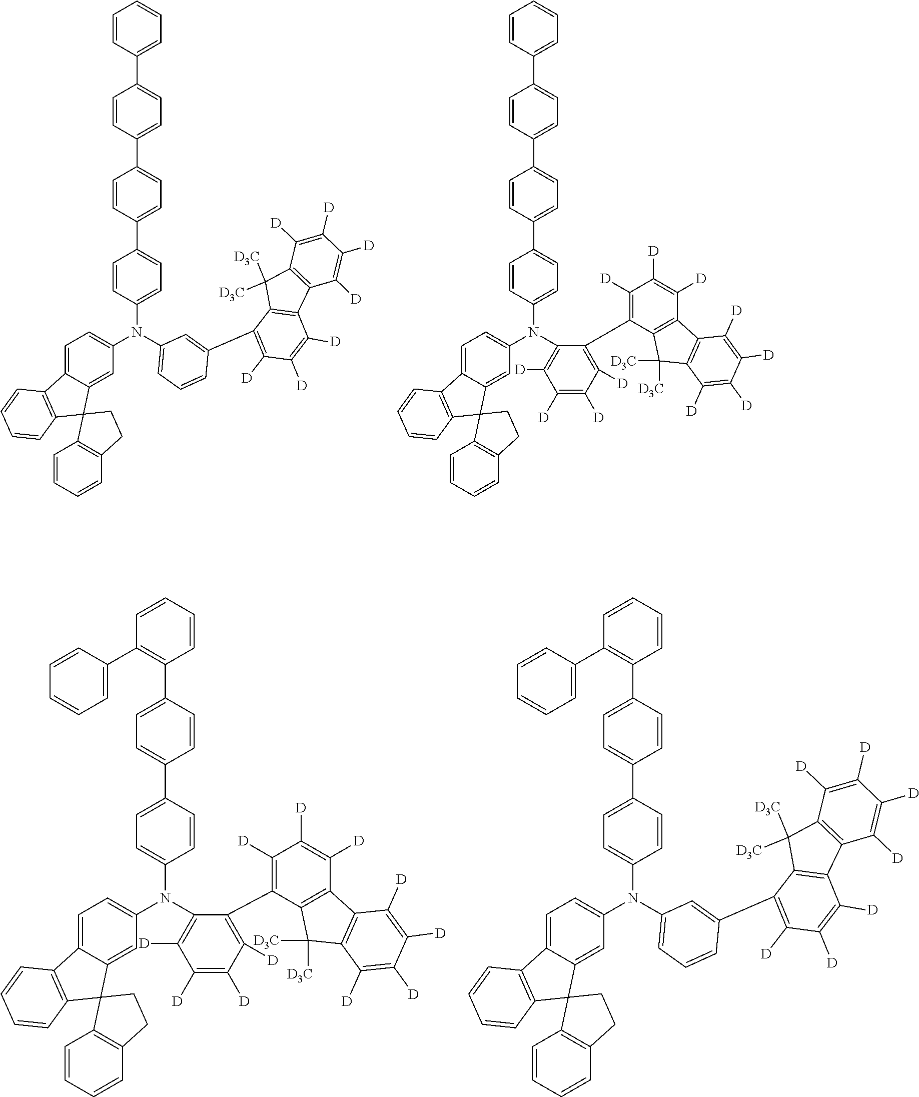

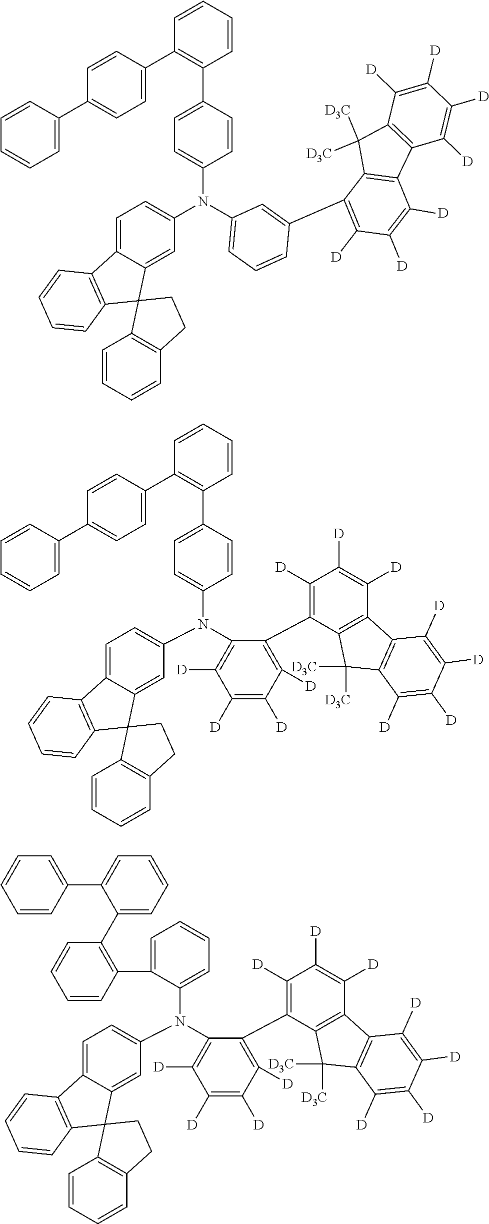

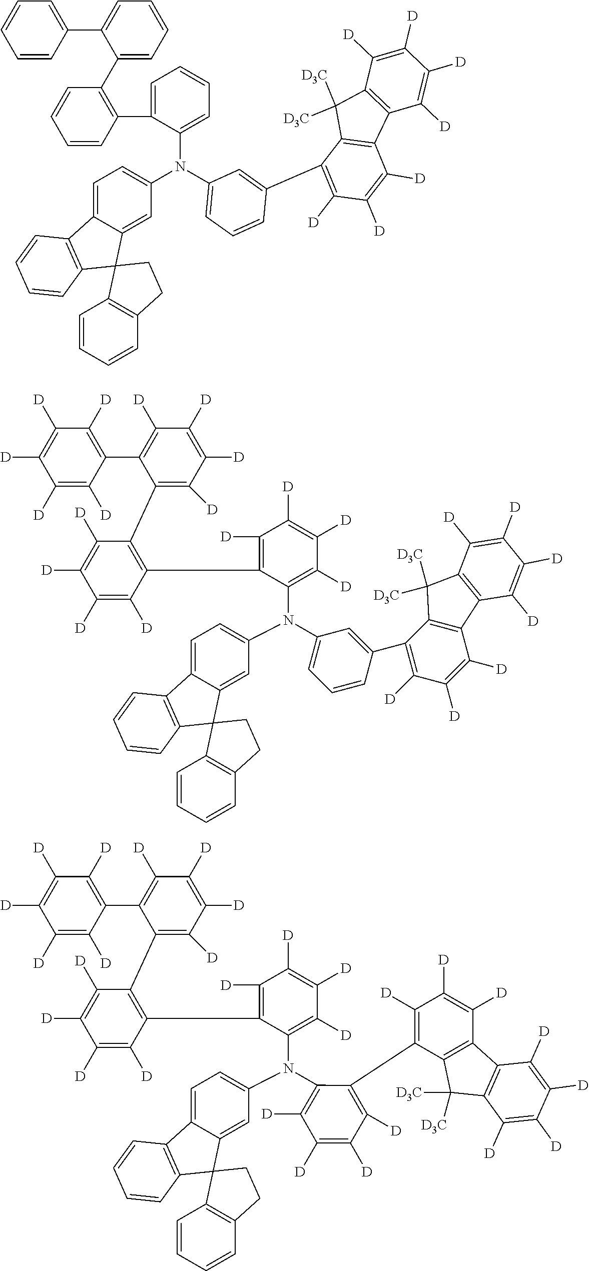

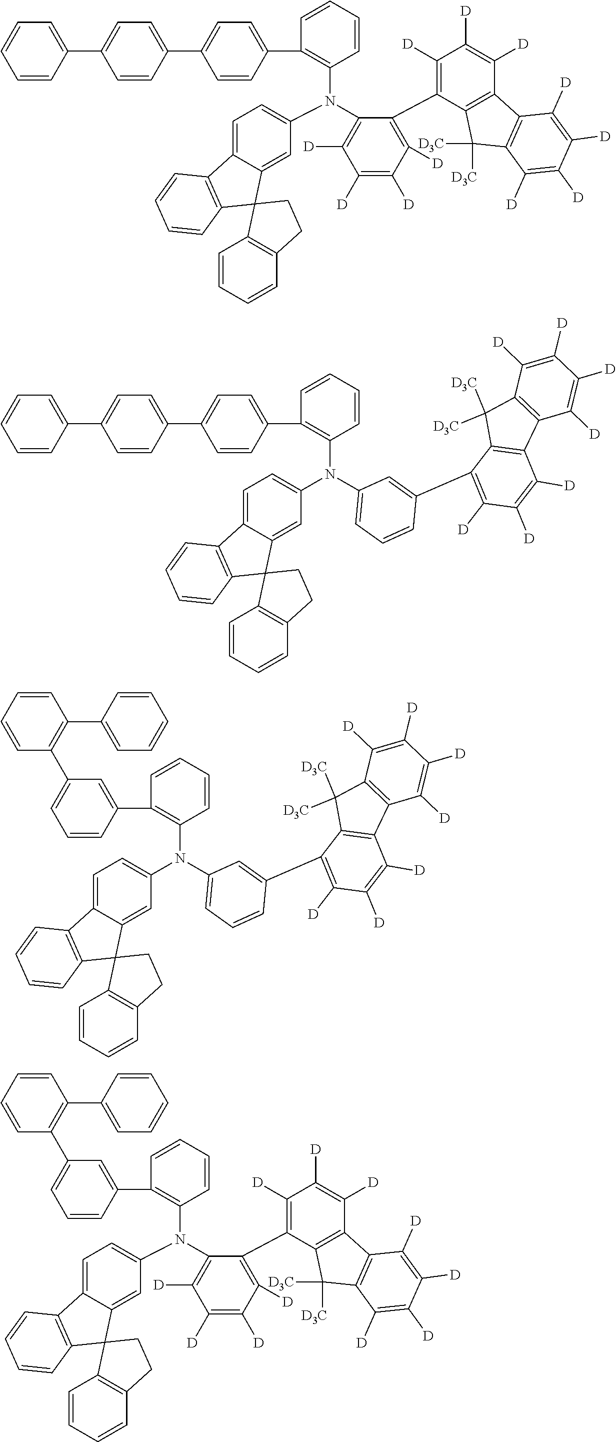

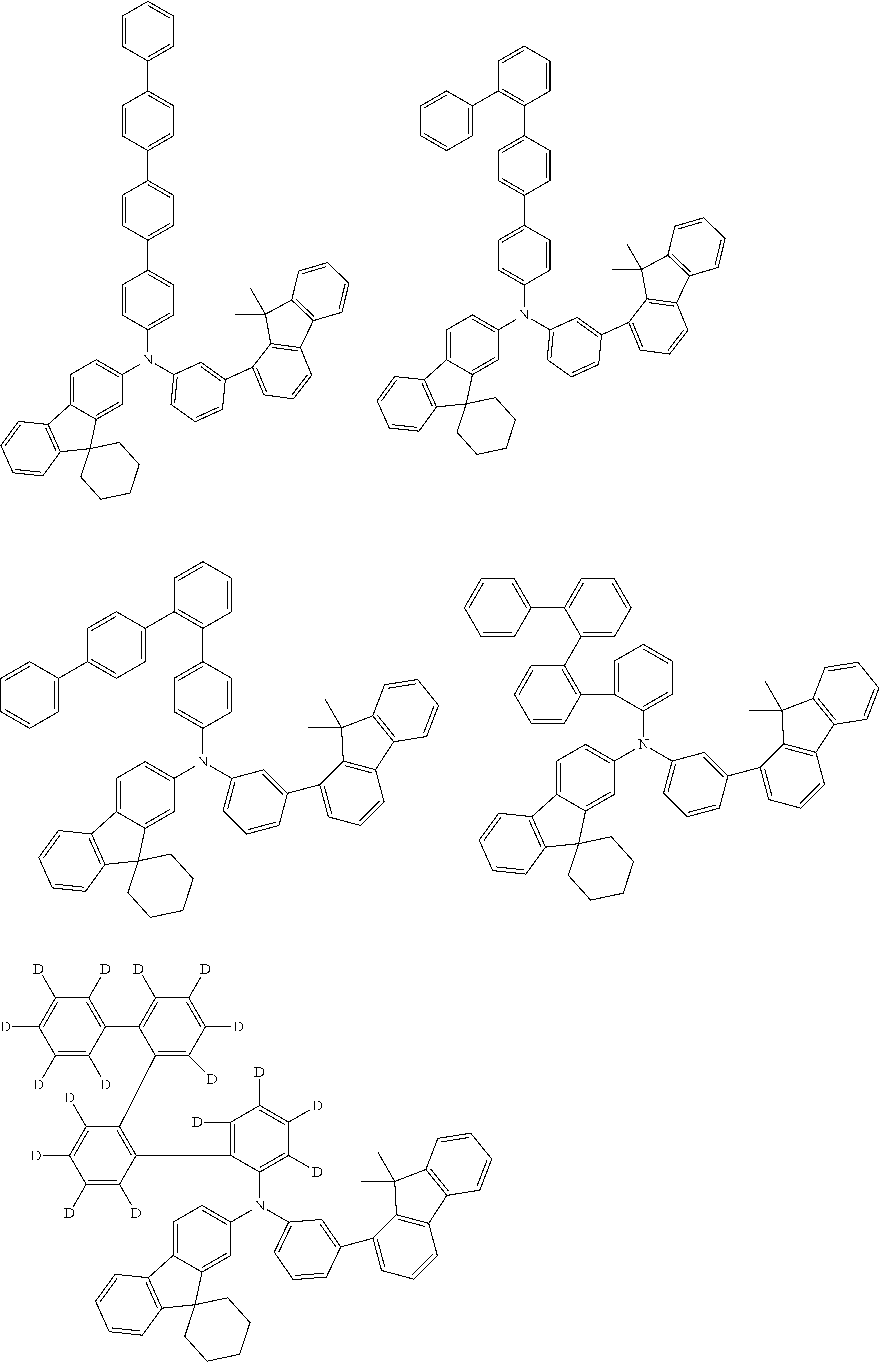

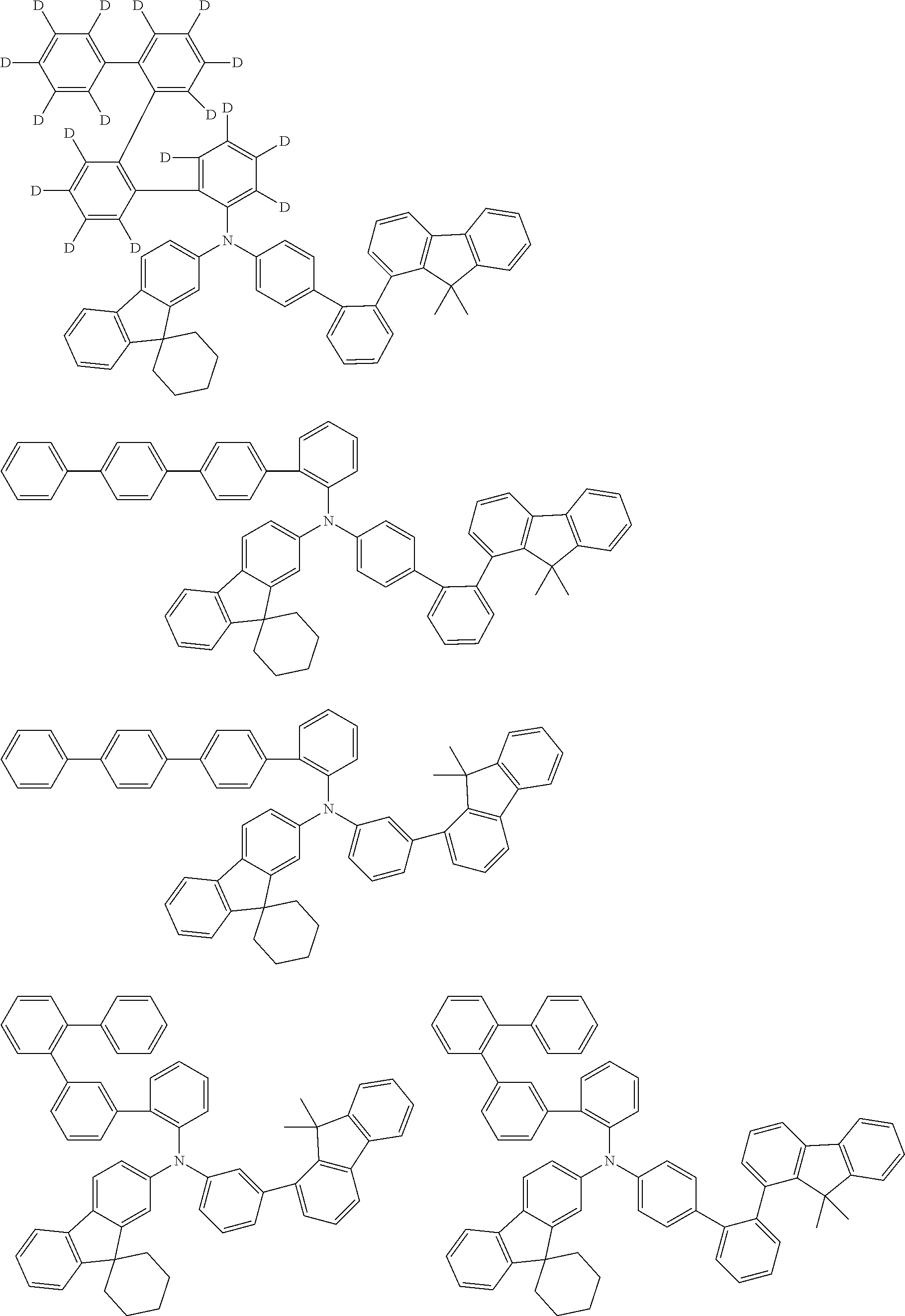

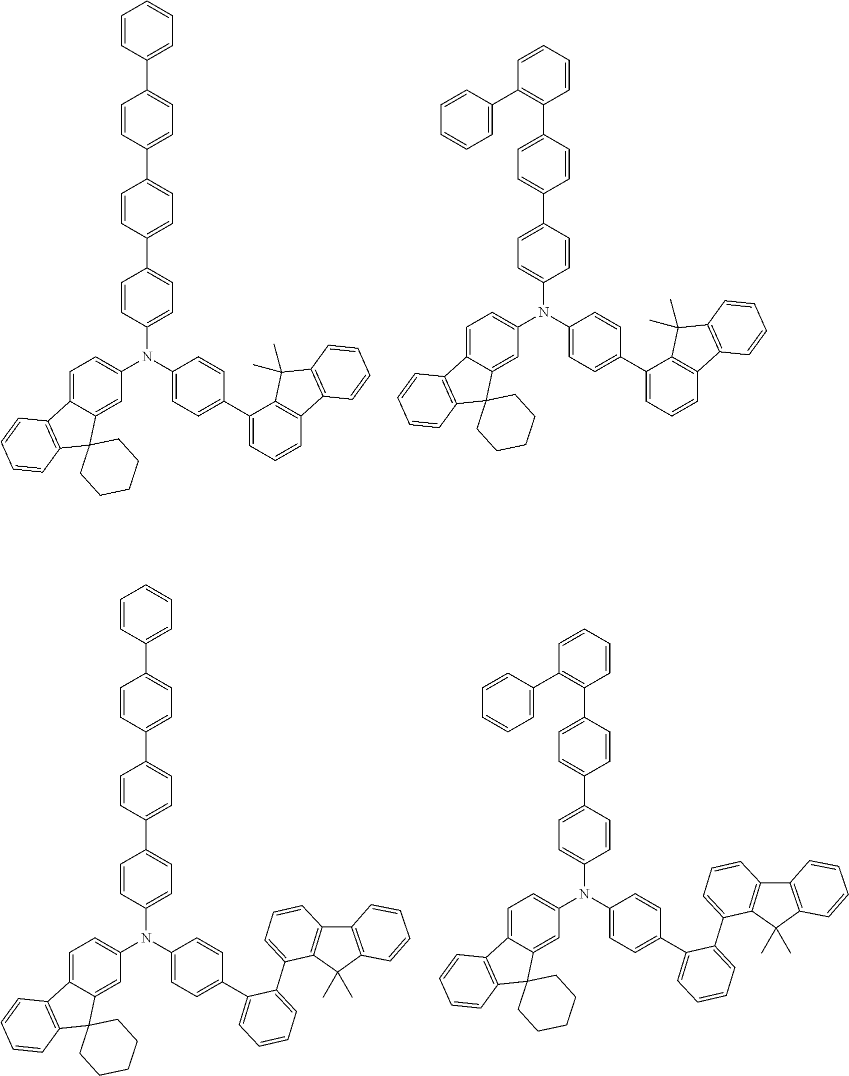

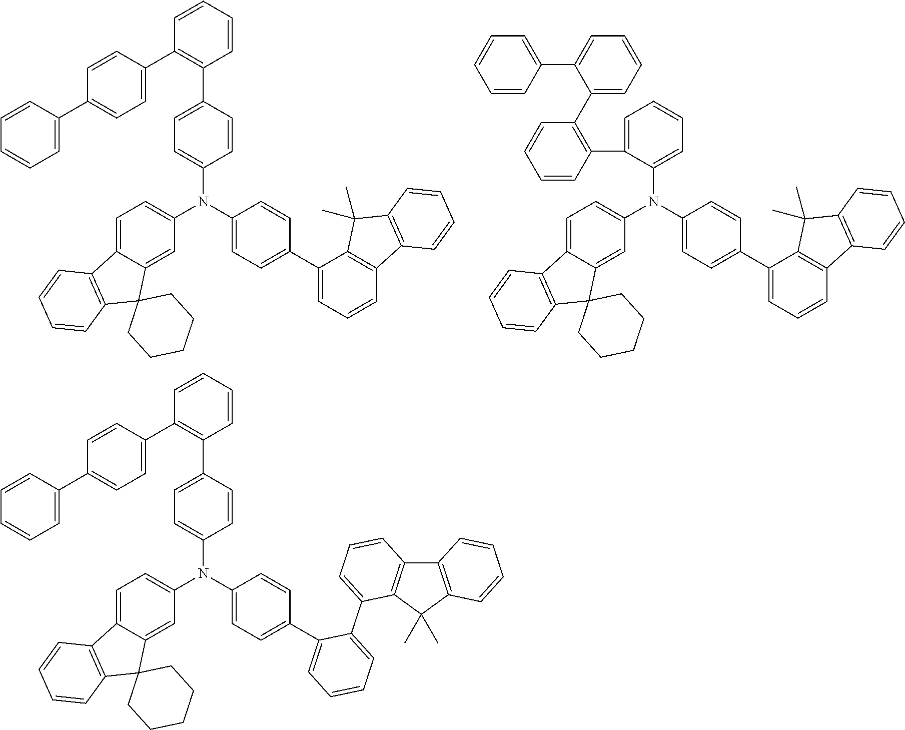

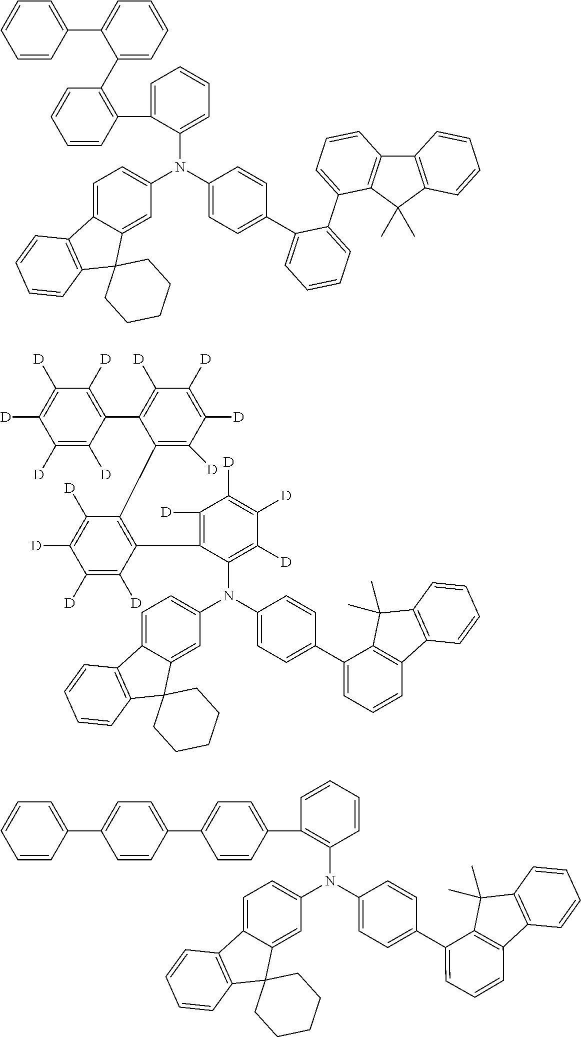

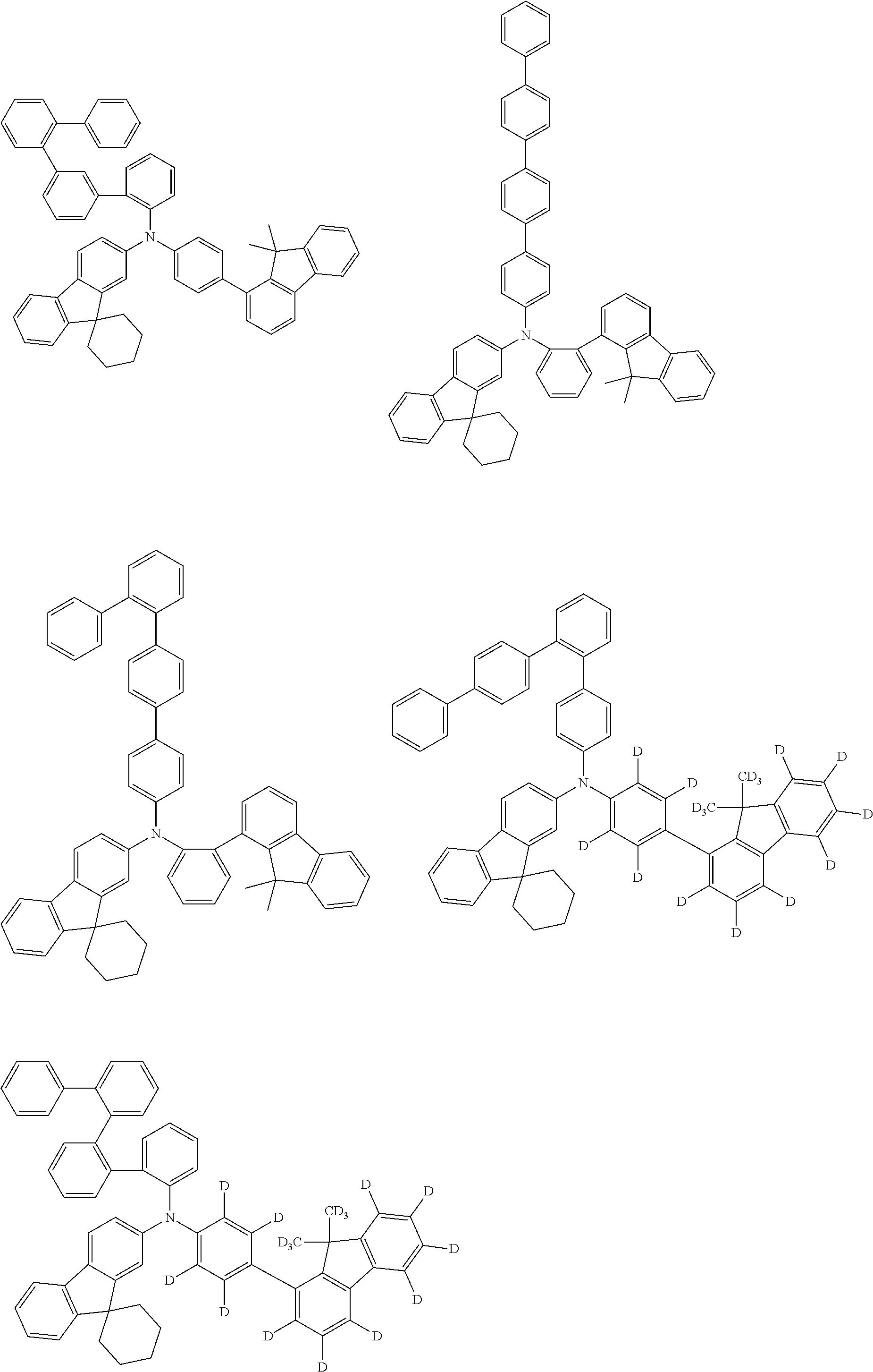

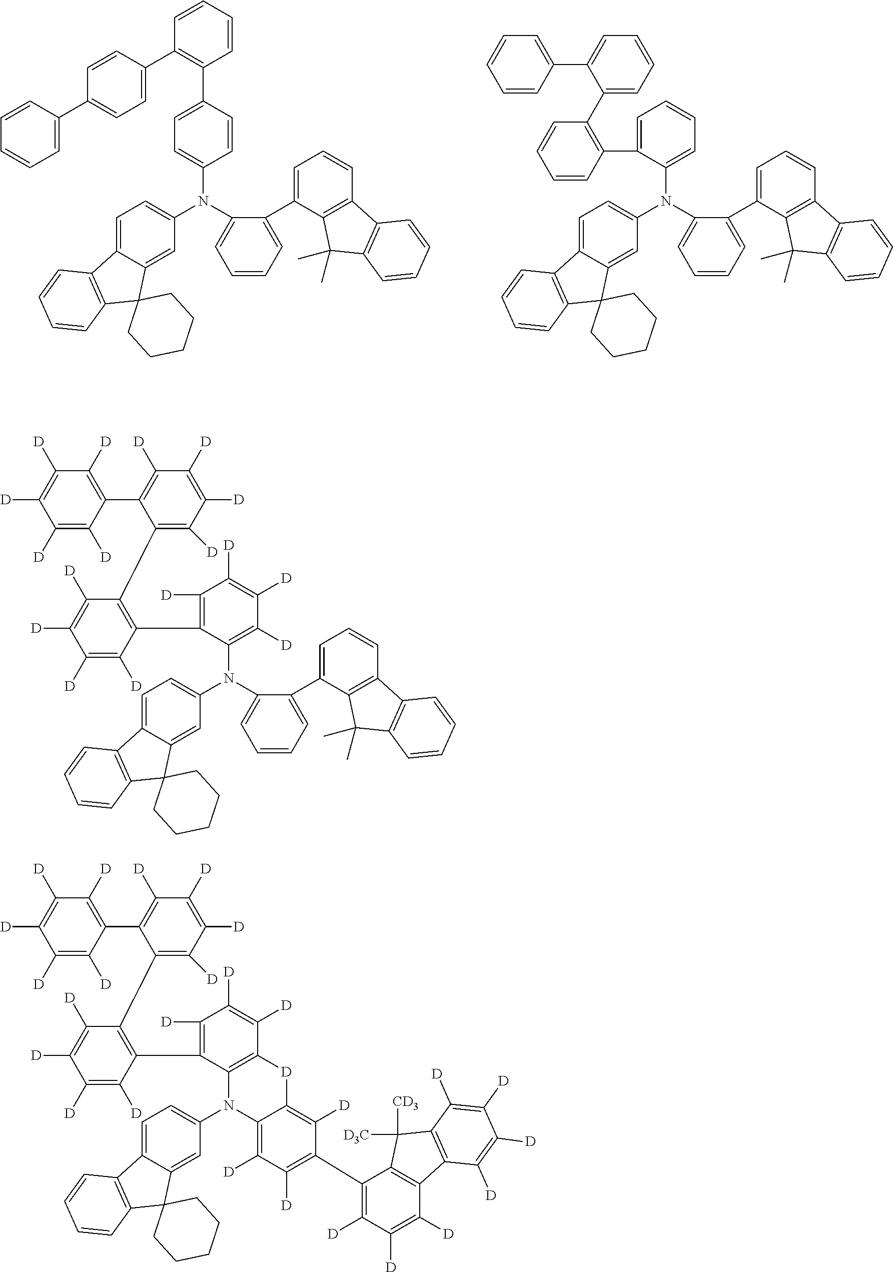

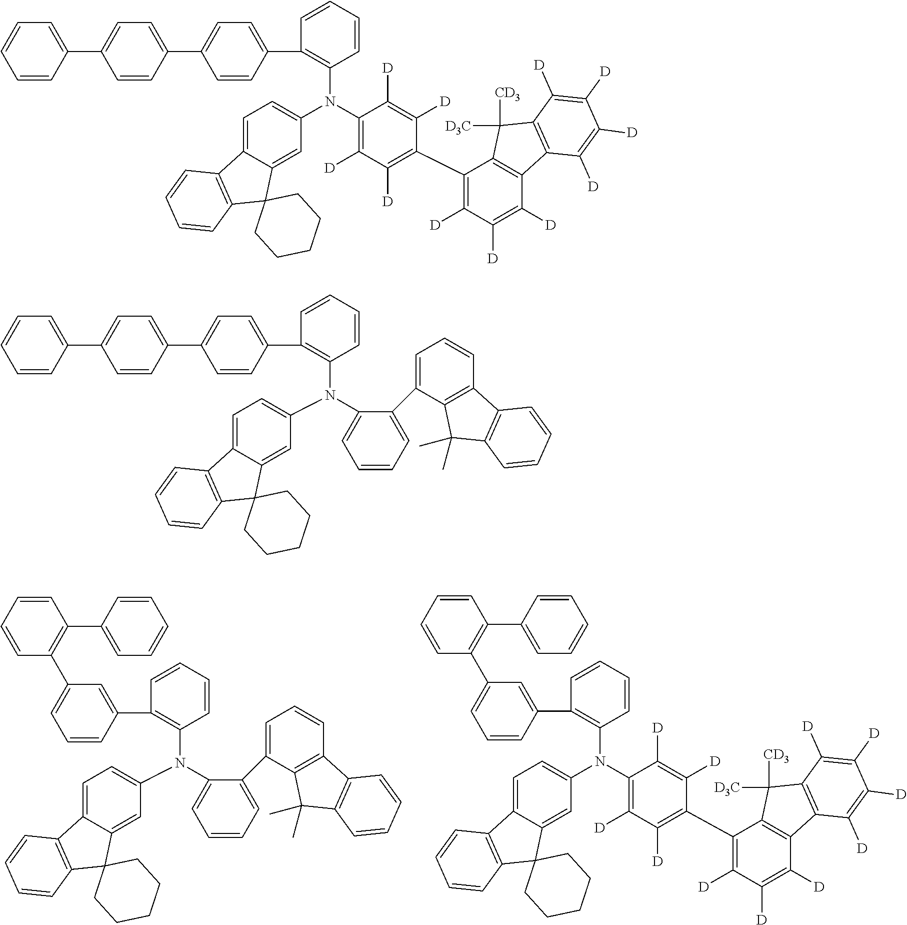

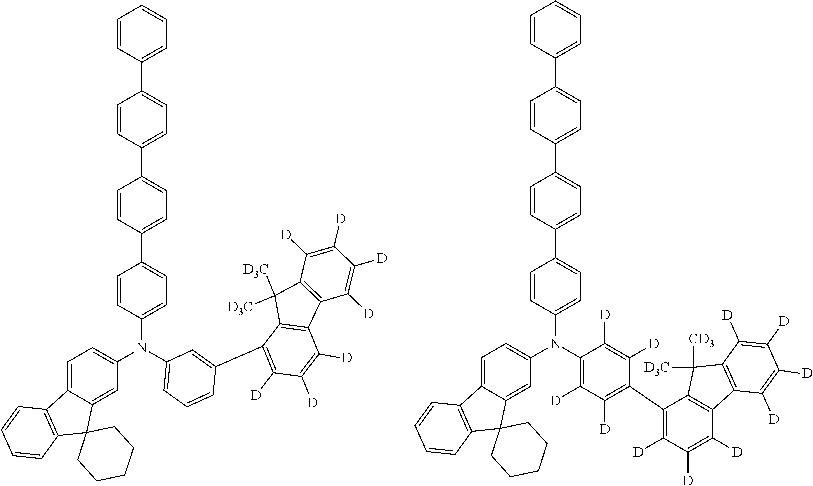 [SMILES: [2H]c1c([2H])c([2H])c2c(c1[2H])-c1c([2H])c([2H])c([2H])c(-c3cccc(N(c4ccc(-c5ccc(-c6ccc(-c7ccccc7)cc6)cc5)cc4)c4ccc5c(c4)C4(CCCCC4)c4ccccc4-5)c3)c1C2(C([2H])([2H])[2H])C([2H])([2H])[2H] ; [2H]c1c([2H])c([2H])c2c(c1[2H])-c1c([2H])c([2H])c([2H])c(-c3c([2H])c([2H])c(N(c4ccc(-c5ccc(-c6ccc(-c7ccccc7)cc6)cc5)cc4)c4ccc5c(c4)C4(CCCCC4)c4ccccc4-5)c([2H])c3[2H])c1C2(C([2H])([2H])[2H])C([2H])([2H])[2H]]

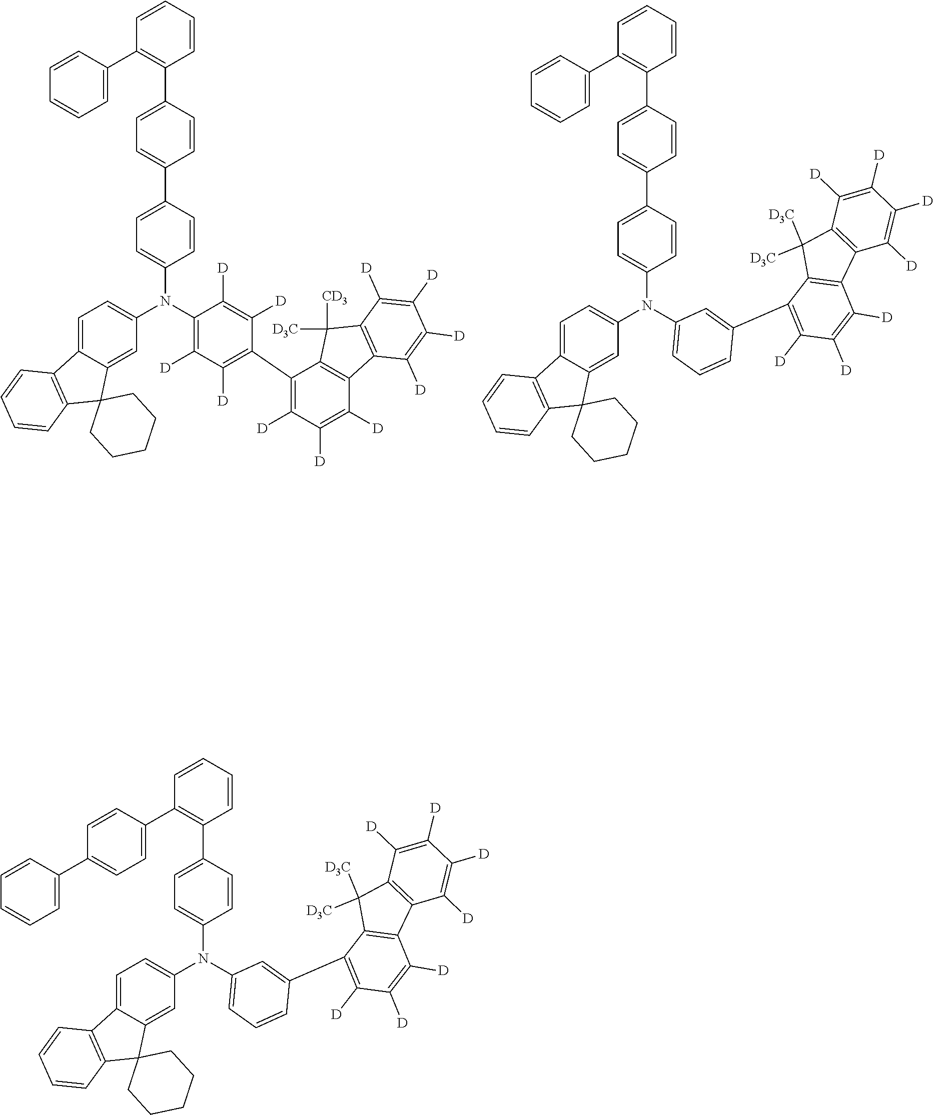

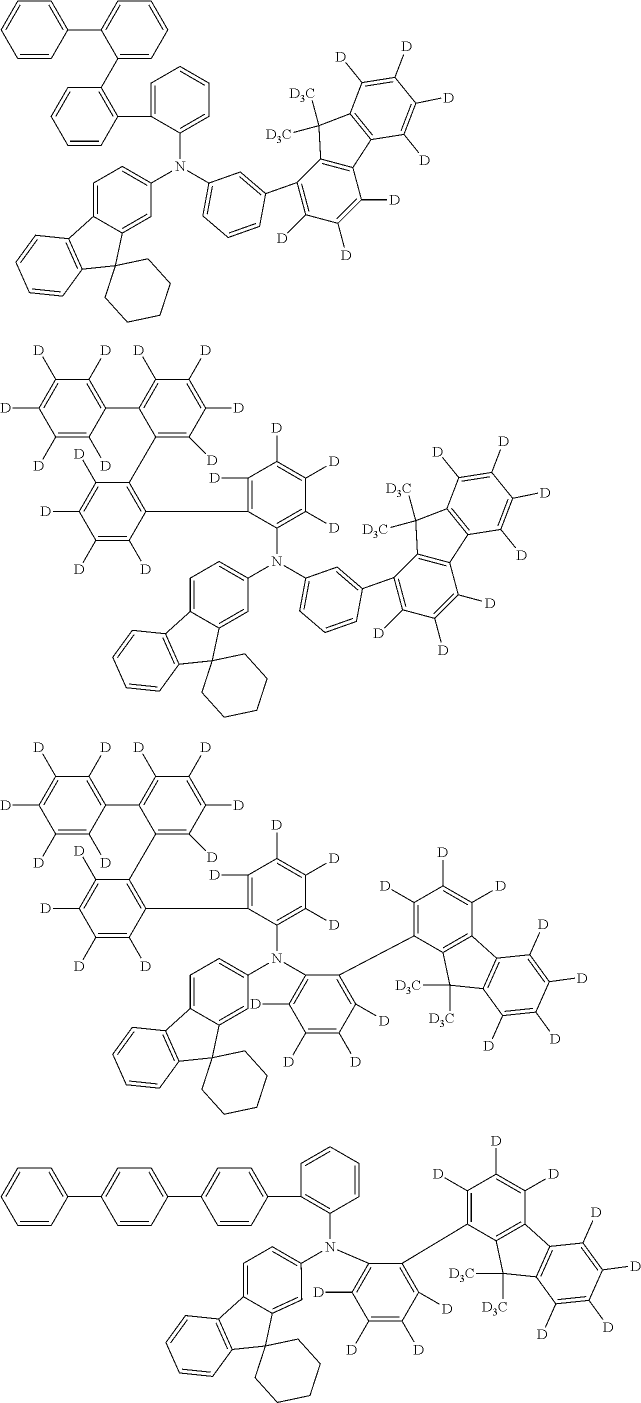

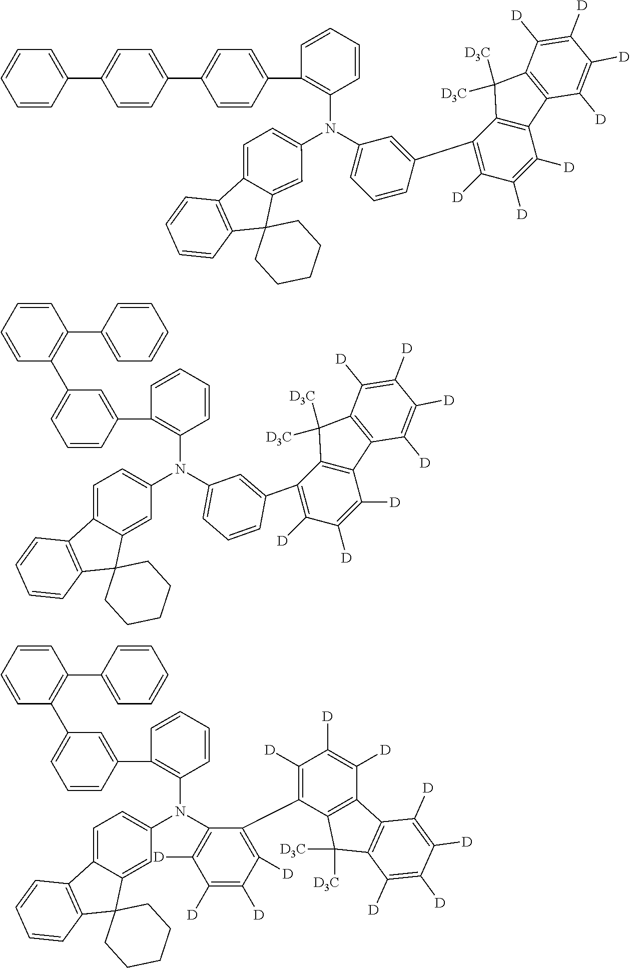

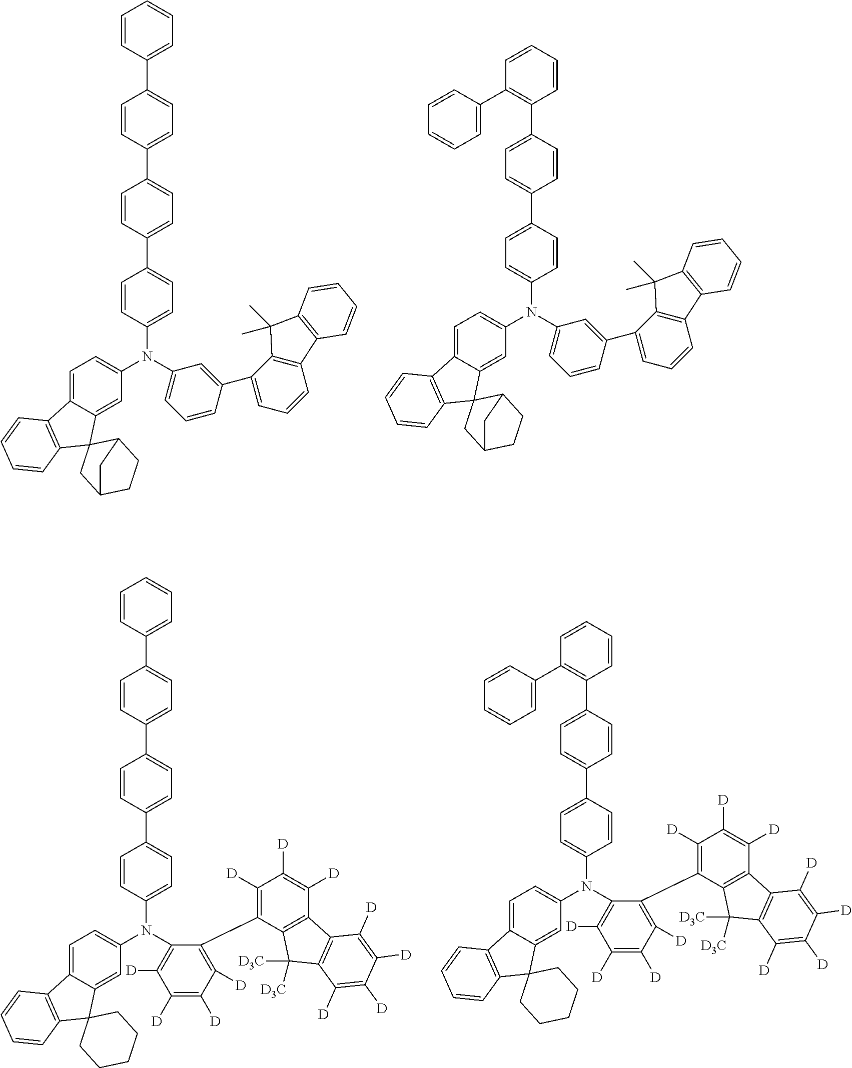

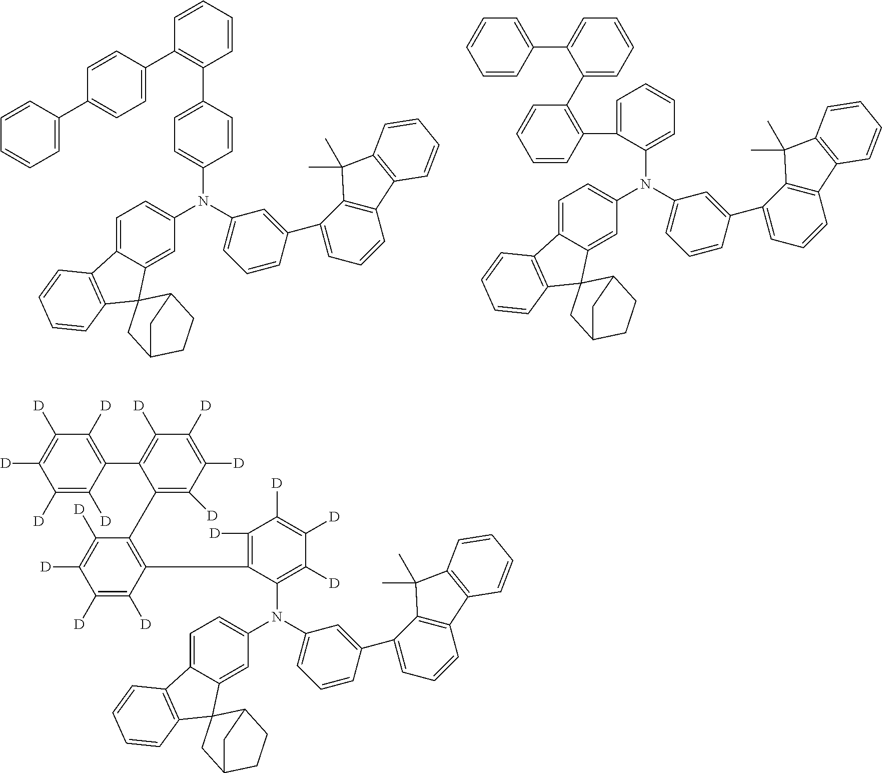 [SMILES: CC1(C)c2ccccc2-c2cccc(-c3cccc(N(c4ccc(-c5ccccc5-c5ccc(-c6ccccc6)cc5)cc4)c4ccc5c(c4)C4(CC6CCC4C6)c4ccccc4-5)c3)c21 ; CC1(C)c2ccccc2-c2cccc(-c3cccc(N(c4ccc5c(c4)C4(CC6CCC4C6)c4ccccc4-5)c4ccccc4-c4ccccc4-c4ccccc4-c4ccccc4)c3)c21 ; [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2cccc(-c3cccc4c3C(C)(C)c3ccccc3-4)c2)c2ccc3c(c2)C2(CC4CCC2C4)c2ccccc2-3)c([2H])c1[2H]]

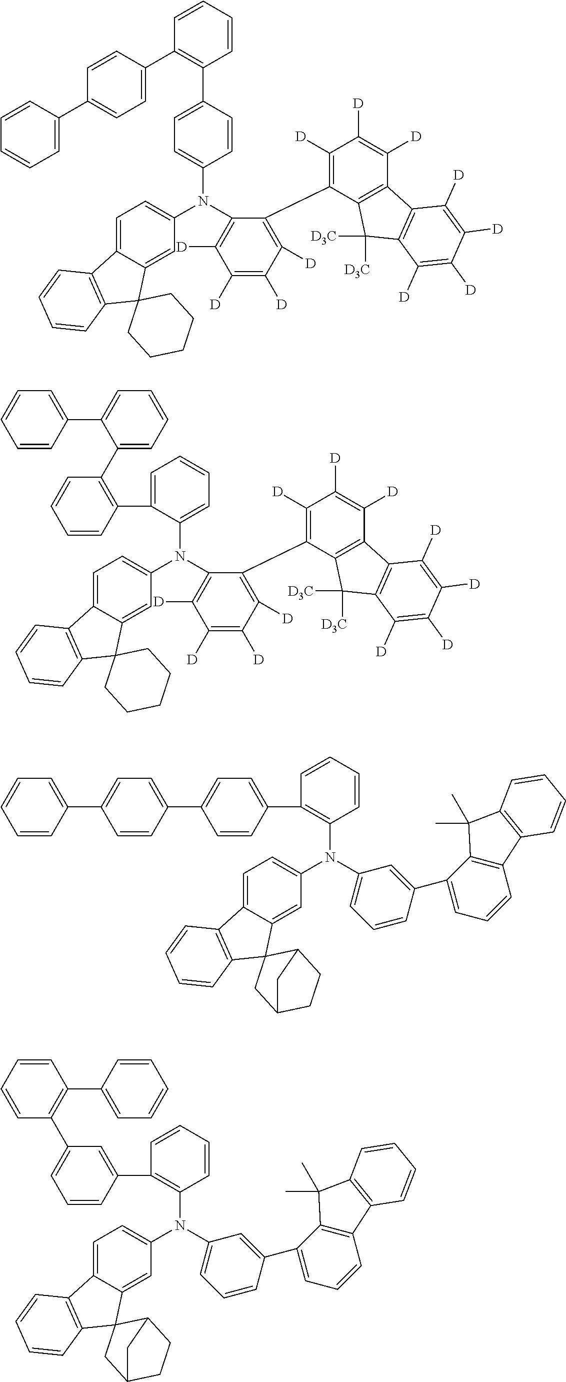

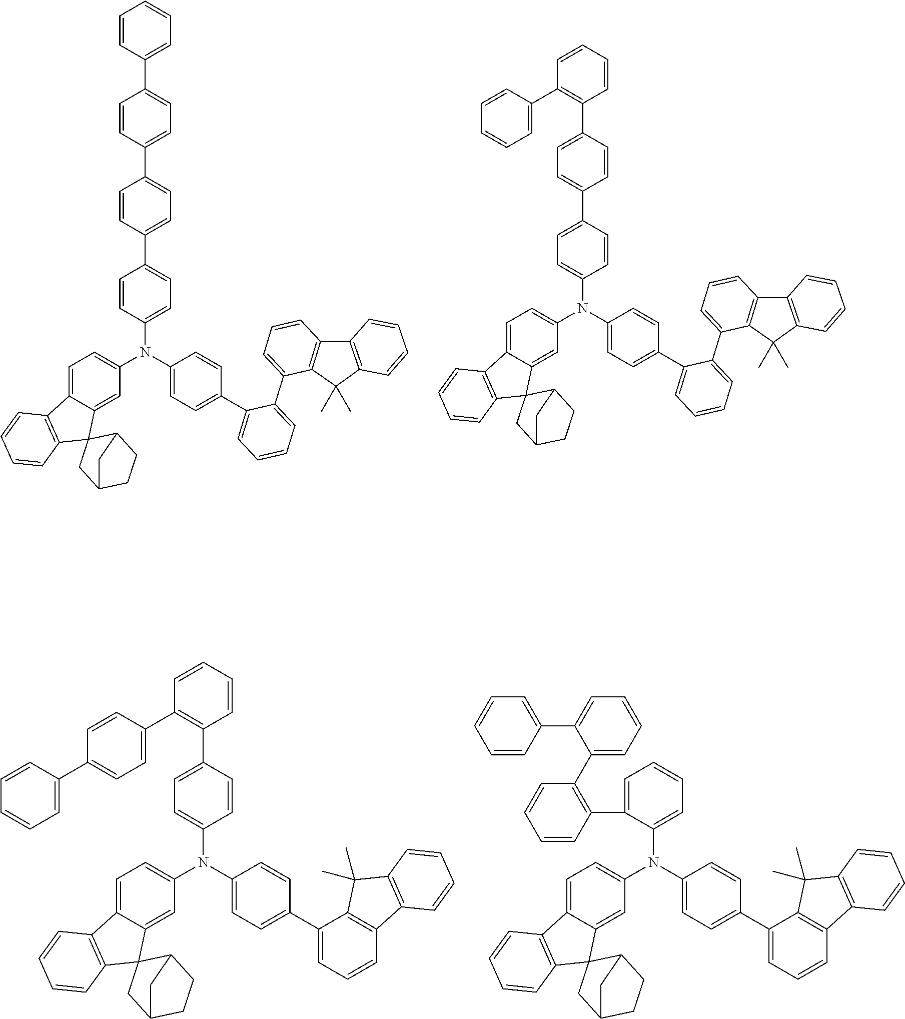 [SMILES: CC1(C)c2ccccc2-c2cccc(-c3ccccc3-c3ccc(N(c4ccc(-c5ccc(-c6ccc(-c7ccccc7)cc6)cc5)cc4)c4ccc5c(c4)C4(CC6CCC4C6)c4ccccc4-5)cc3)c21 ; CC1(C)c2ccccc2-c2cccc(-c3ccccc3-c3ccc(N(c4ccc(-c5ccc(-c6ccccc6-c6ccccc6)cc5)cc4)c4ccc5c(c4)C4(CC6CCC4C6)c4ccccc4-5)cc3)c21 ; CC1(C)c2ccccc2-c2cccc(-c3ccc(N(c4ccc(-c5ccccc5-c5ccc(-c6ccccc6)cc5)cc4)c4ccc5c(c4)C4(CC6CCC4C6)c4ccccc4-5)cc3)c21 ; CC1(C)c2ccccc2-c2cccc(-c3ccc(N(c4ccc5c(c4)C4(CC6CCC4C6)c4ccccc4-5)c4ccccc4-c4ccccc4-c4ccccc4-c4ccccc4)cc3)c21]

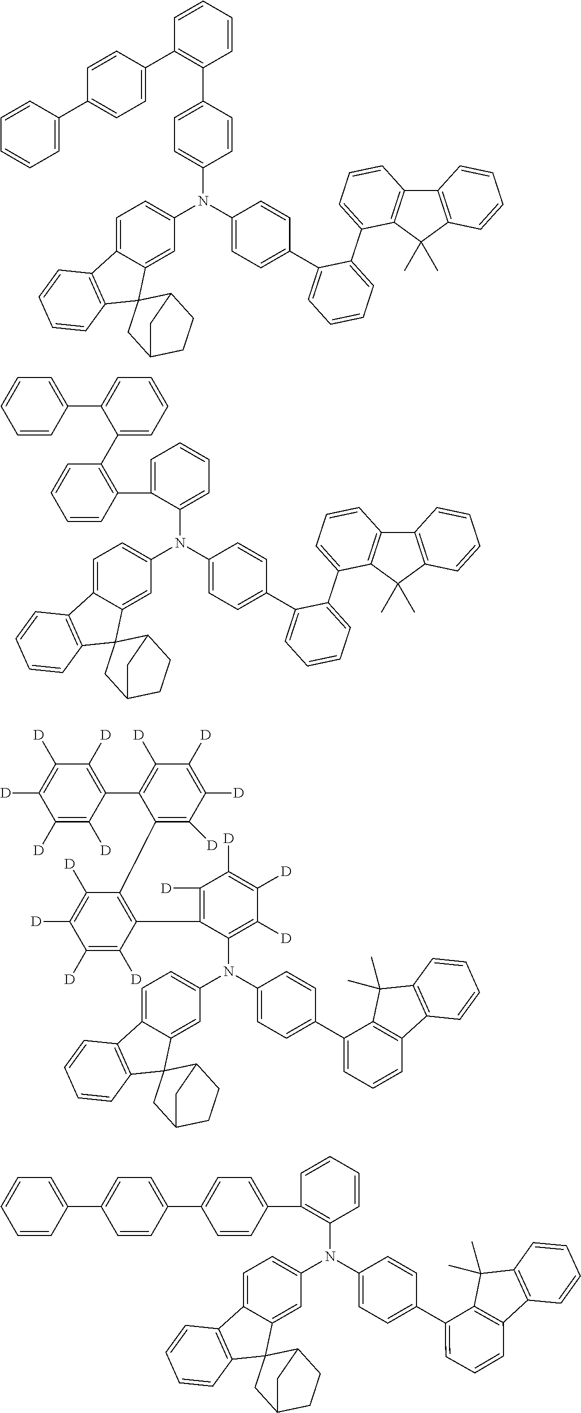

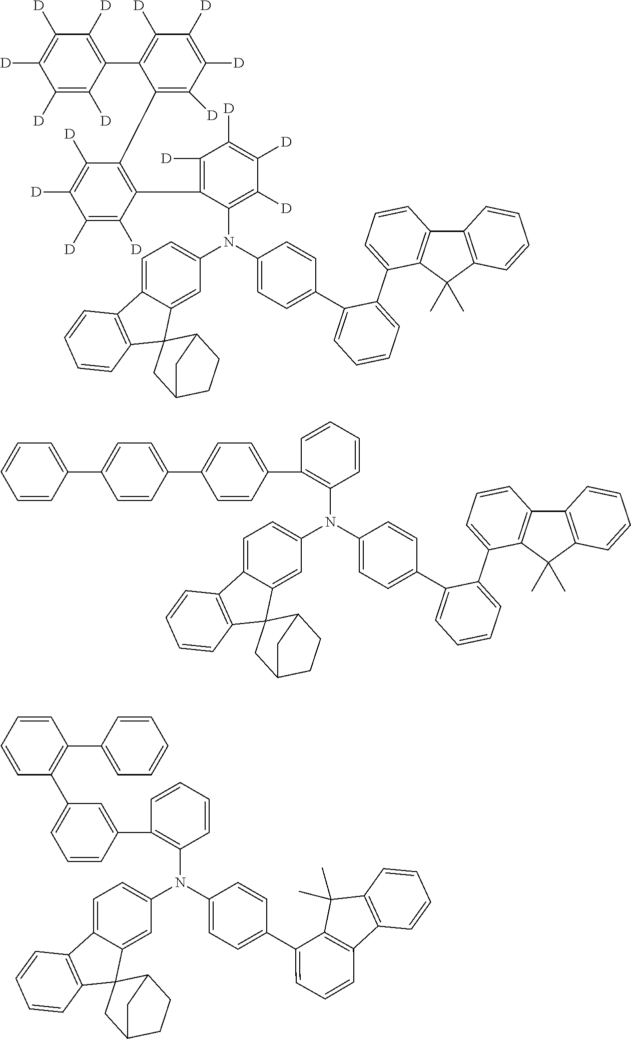 [SMILES: [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2ccc(-c3ccccc3-c3cccc4c3C(C)(C)c3ccccc3-4)cc2)c2ccc3c(c2)C2(CC4CCC2C4)c2ccccc2-3)c([2H])c1[2H] ; CC1(C)c2ccccc2-c2cccc(-c3ccccc3-c3ccc(N(c4ccc5c(c4)C4(CC6CCC4C6)c4ccccc4-5)c4ccccc4-c4ccc(-c5ccc(-c6ccccc6)cc5)cc4)cc3)c21 ; CC1(C)c2ccccc2-c2cccc(-c3ccc(N(c4ccc5c(c4)C4(CC6CCC4C6)c4ccccc4-5)c4ccccc4-c4cccc(-c5ccccc5-c5ccccc5)c4)cc3)c21]

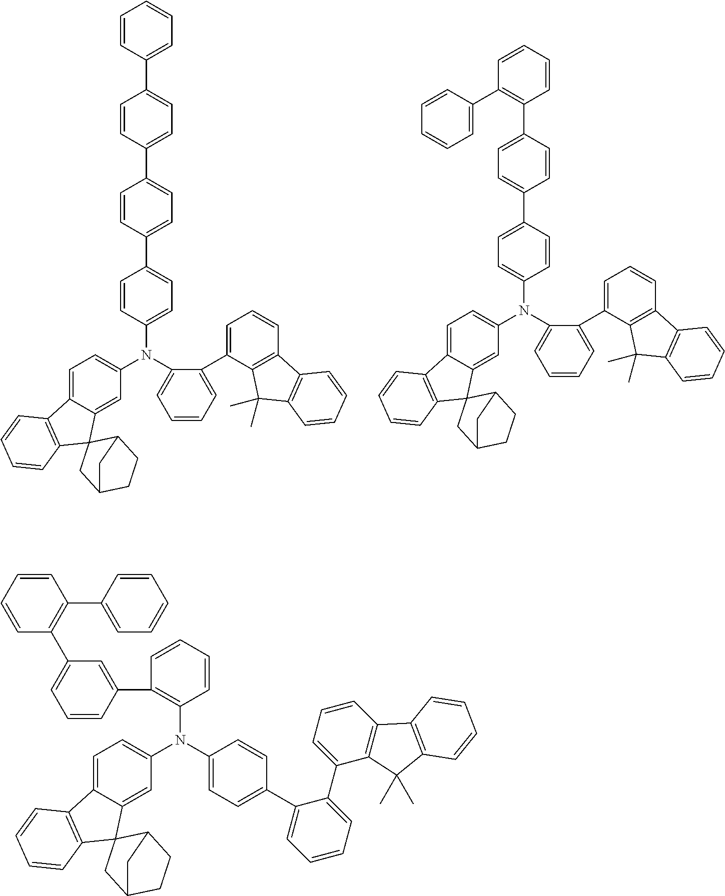

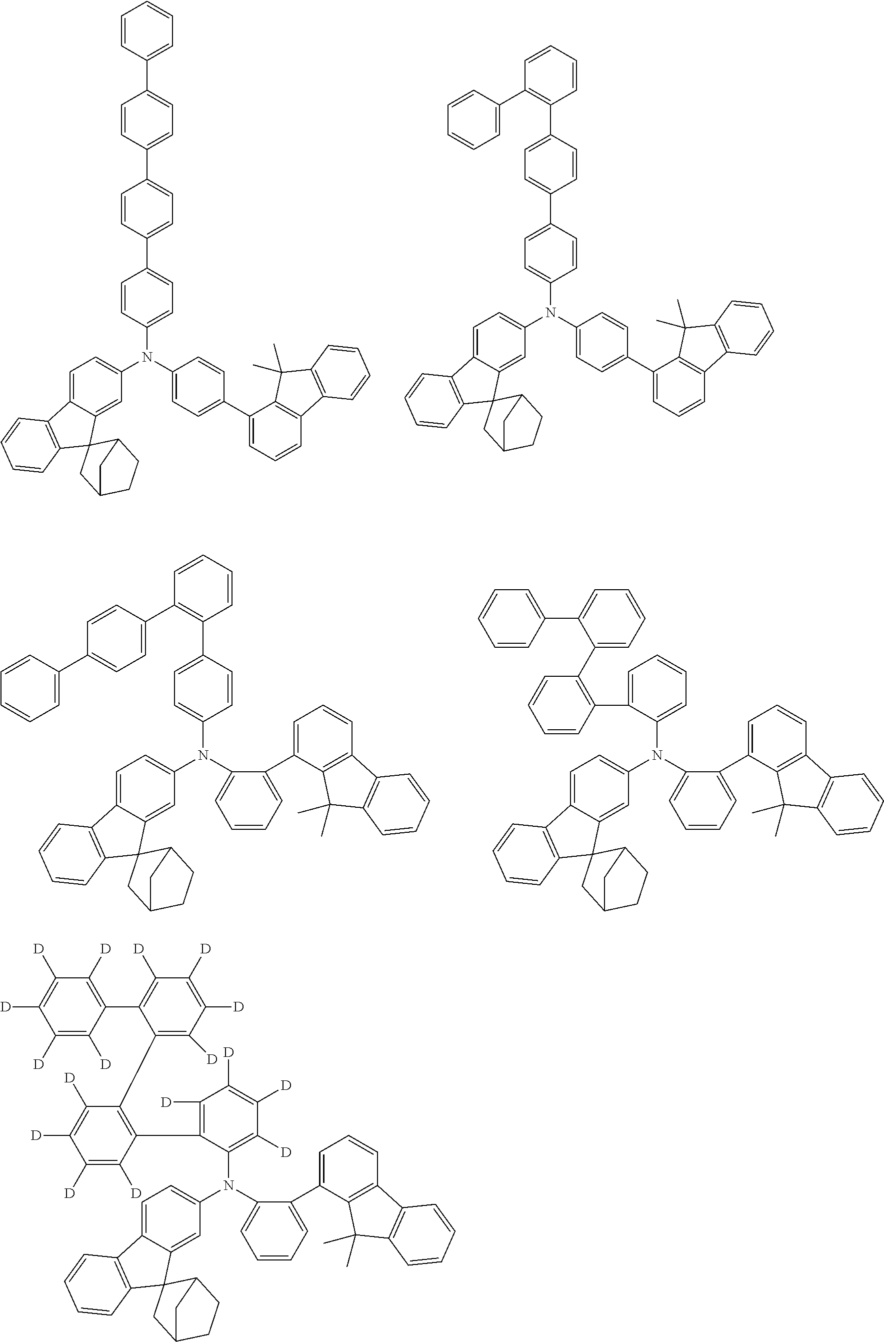

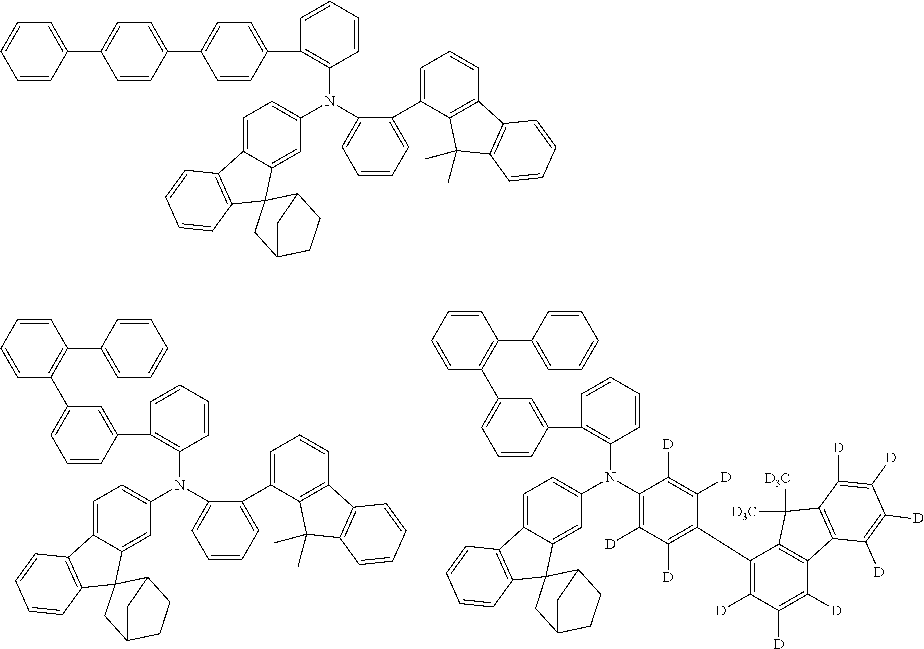 [SMILES: CC1(C)c2ccccc2-c2cccc(-c3ccccc3N(c3ccc4c(c3)C3(CC5CCC3C5)c3ccccc3-4)c3ccccc3-c3ccc(-c4ccc(-c5ccccc5)cc4)cc3)c21 ; CC1(C)c2ccccc2-c2cccc(-c3ccccc3N(c3ccc4c(c3)C3(CC5CCC3C5)c3ccccc3-4)c3ccccc3-c3cccc(-c4ccccc4-c4ccccc4)c3)c21 ; [2H]c1c([2H])c([2H])c2c(c1[2H])-c1c([2H])c([2H])c([2H])c(-c3c([2H])c([2H])c(N(c4ccc5c(c4)C4(CC6CCC4C6)c4ccccc4-5)c4ccccc4-c4cccc(-c5ccccc5-c5ccccc5)c4)c([2H])c3[2H])c1C2(C([2H])([2H])[2H])C([2H])([2H])[2H]]

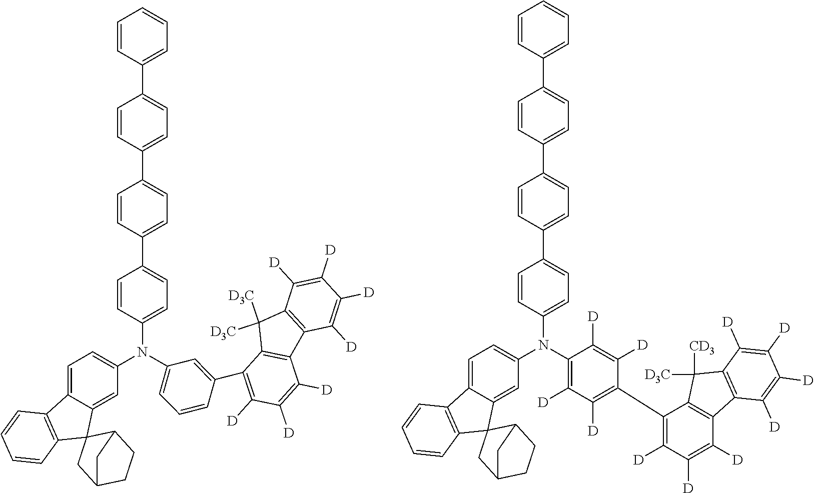 [SMILES: [2H]c1c([2H])c([2H])c2c(c1[2H])-c1c([2H])c([2H])c([2H])c(-c3cccc(N(c4ccc(-c5ccc(-c6ccc(-c7ccccc7)cc6)cc5)cc4)c4ccc5c(c4)C4(CC6CCC4C6)c4ccccc4-5)c3)c1C2(C([2H])([2H])[2H])C([2H])([2H])[2H] ; [2H]c1c([2H])c([2H])c2c(c1[2H])-c1c([2H])c([2H])c([2H])c(-c3c([2H])c([2H])c(N(c4ccc(-c5ccc(-c6ccc(-c7ccccc7)cc6)cc5)cc4)c4ccc5c(c4)C4(CC6CCC4C6)c4ccccc4-5)c([2H])c3[2H])c1C2(C([2H])([2H])[2H])C([2H])([2H])[2H]]

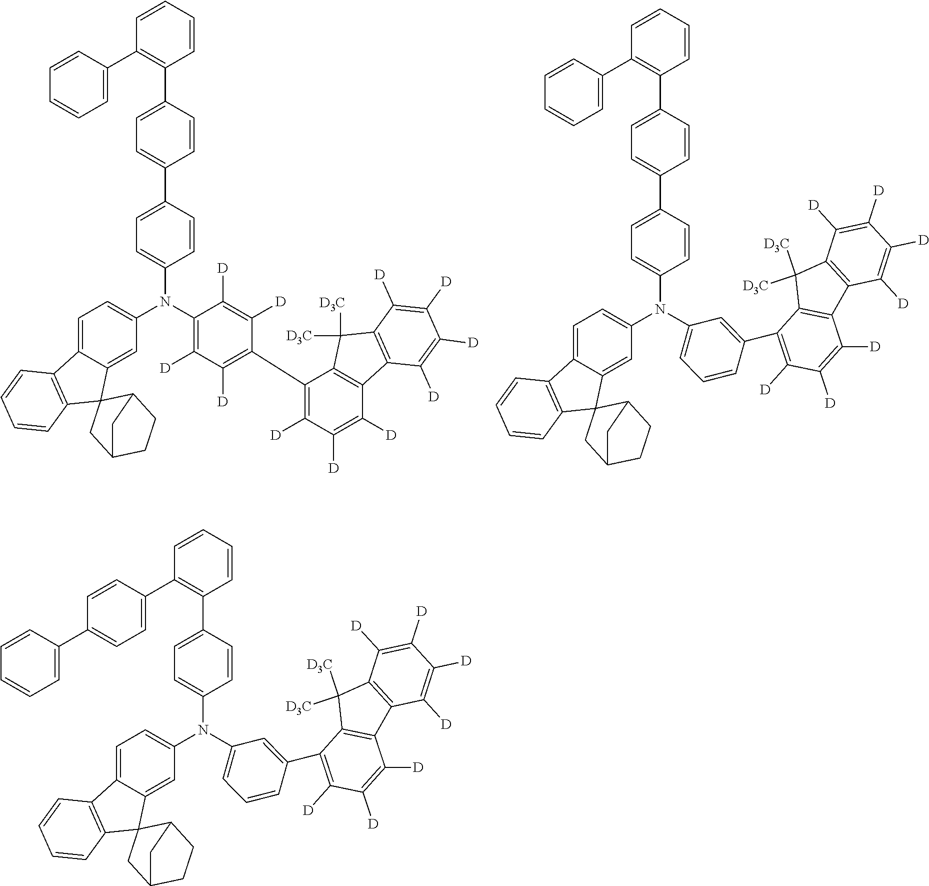

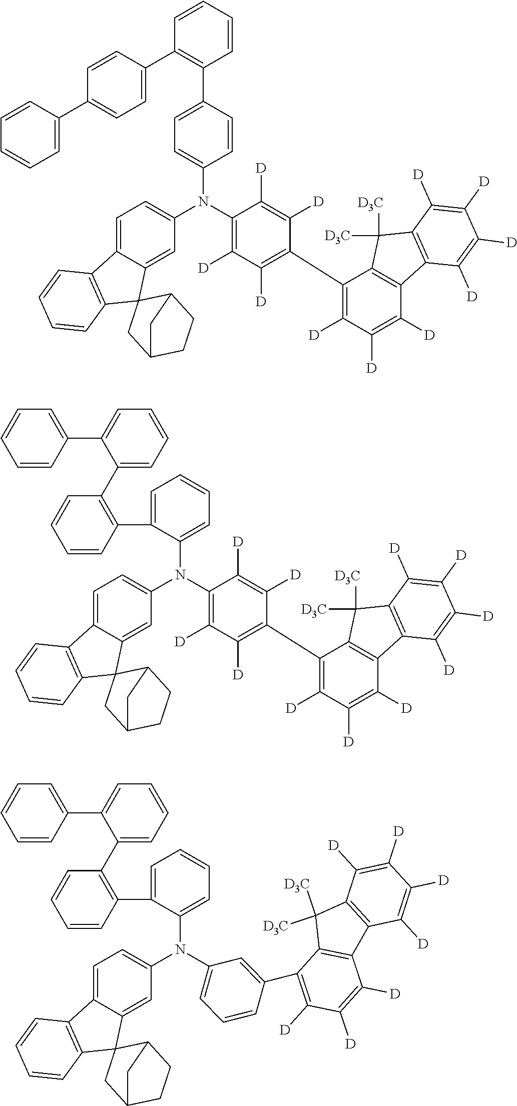

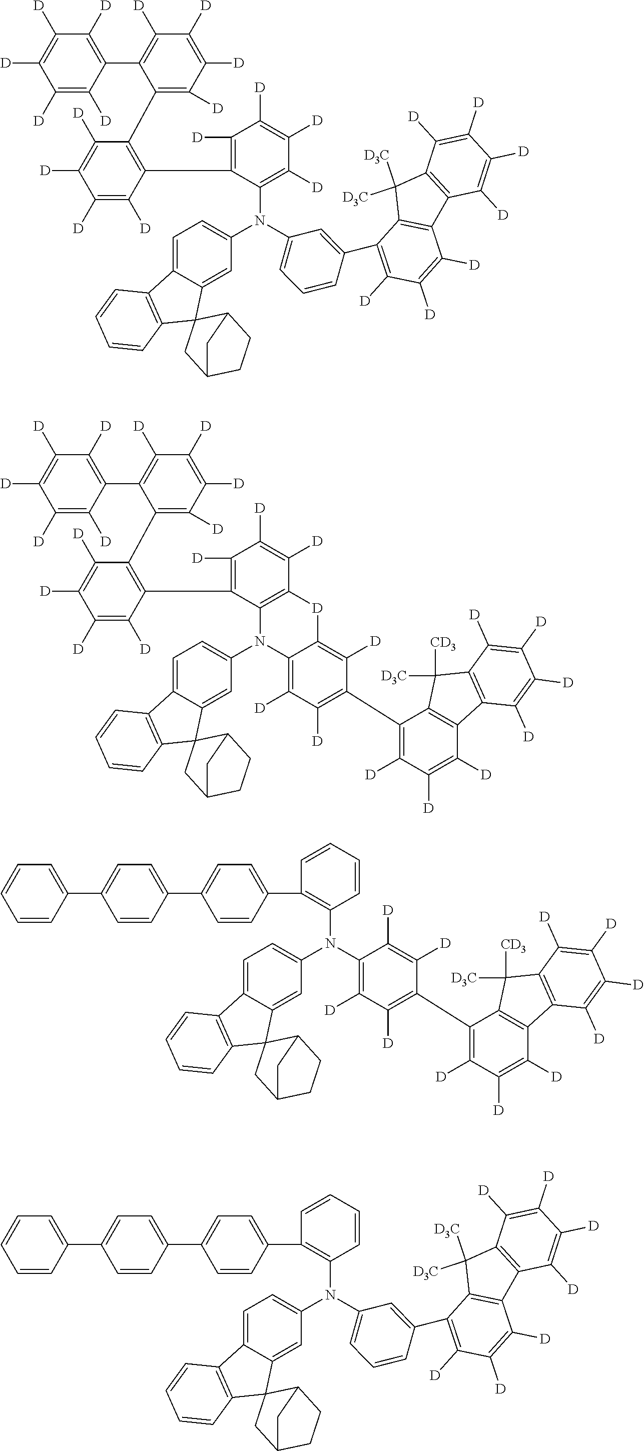

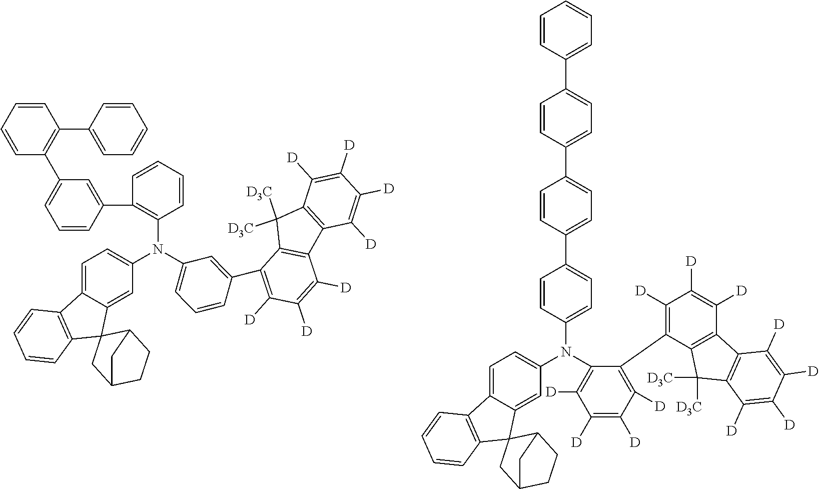 [SMILES: [2H]c1c([2H])c([2H])c2c(c1[2H])-c1c([2H])c([2H])c([2H])c(-c3cccc(N(c4ccc5c(c4)C4(CC6CCC4C6)c4ccccc4-5)c4ccccc4-c4cccc(-c5ccccc5-c5ccccc5)c4)c3)c1C2(C([2H])([2H])[2H])C([2H])([2H])[2H] ; [2H]c1c([2H])c([2H])c(N(c2ccc(-c3ccc(-c4ccc(-c5ccccc5)cc4)cc3)cc2)c2ccc3c(c2)C2(CC4CCC2C4)c2ccccc2-3)c(-c2c([2H])c([2H])c([2H])c3c2C(C([2H])([2H])[2H])(C([2H])([2H])[2H])c2c([2H])c([2H])c([2H])c([2H])c2-3)c1[2H]]

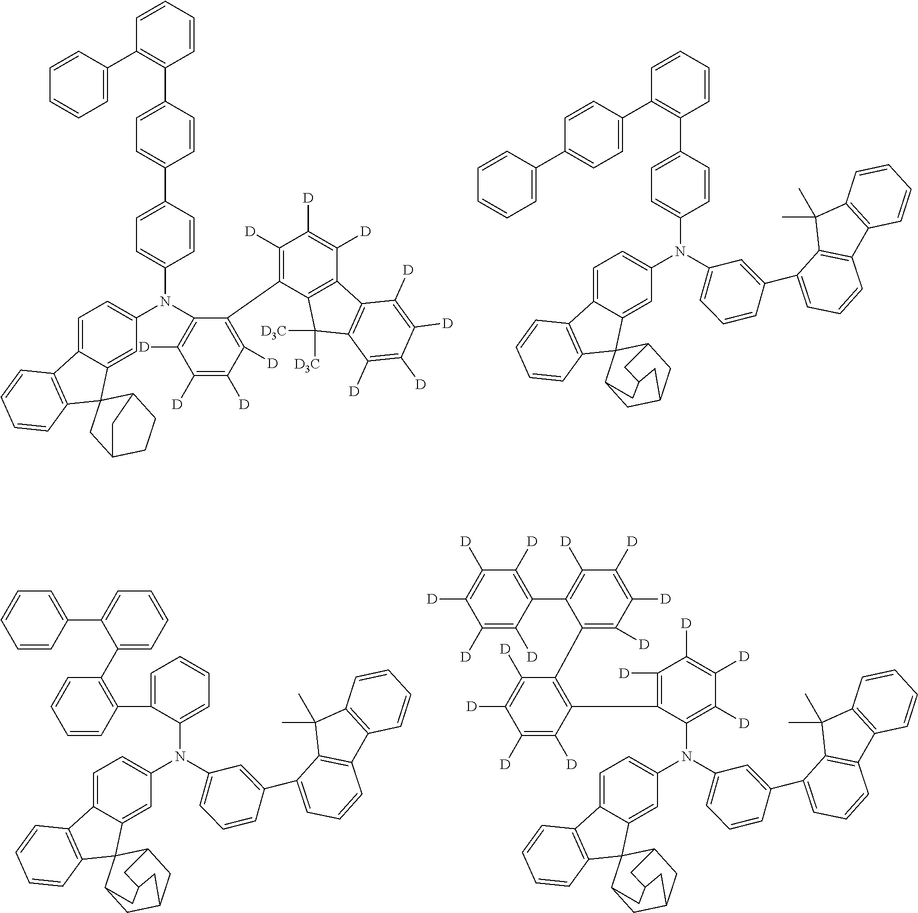

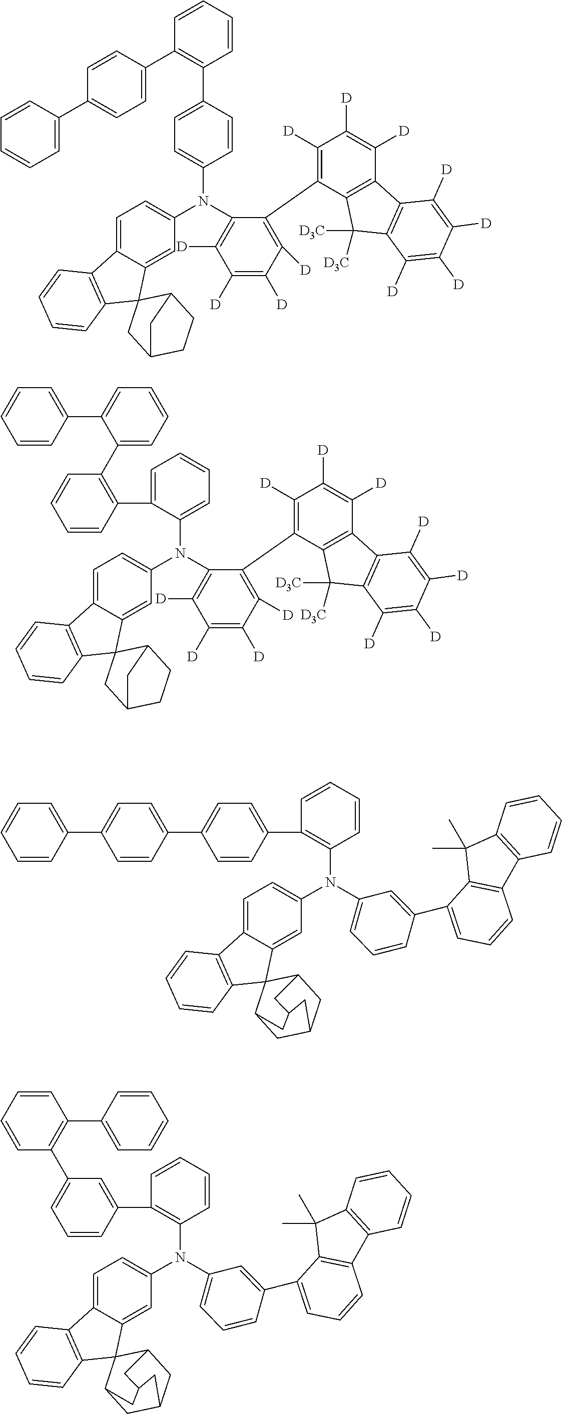

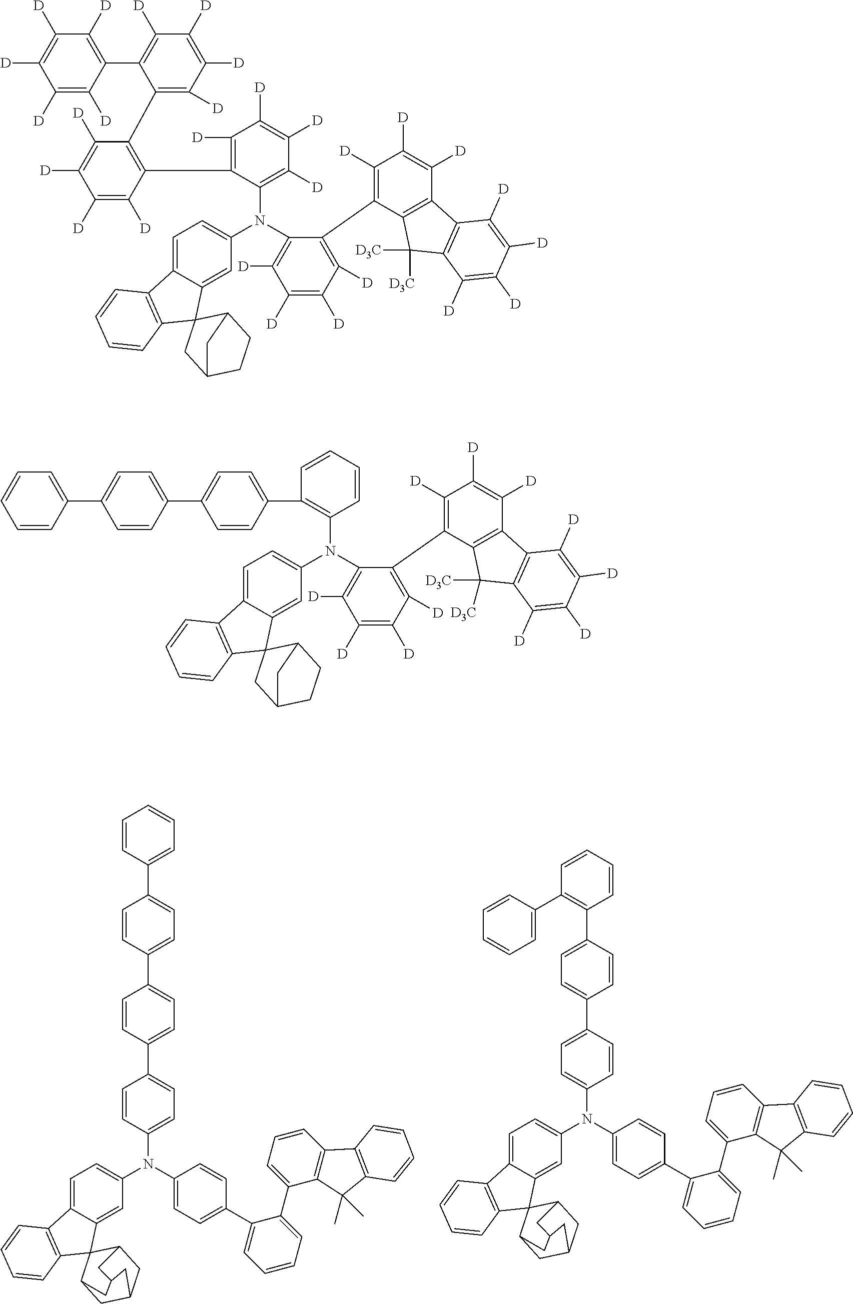

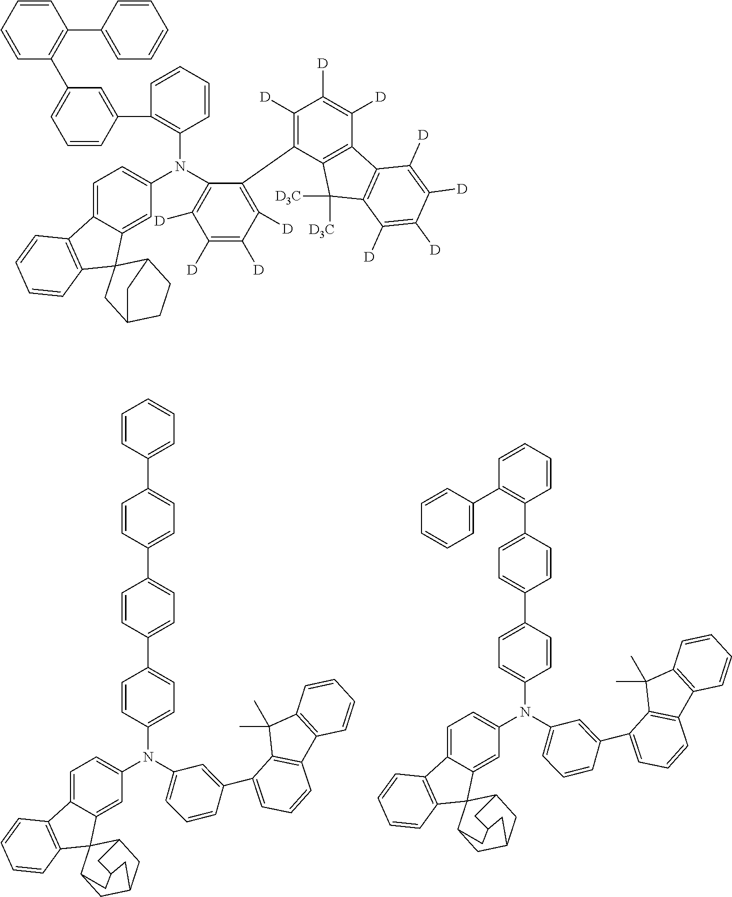 [SMILES: [2H]c1c([2H])c([2H])c(N(c2ccc3c(c2)C2(CC4CCC2C4)c2ccccc2-3)c2ccccc2-c2cccc(-c3ccccc3-c3ccccc3)c2)c(-c2c([2H])c([2H])c([2H])c3c2C(C([2H])([2H])[2H])(C([2H])([2H])[2H])c2c([2H])c([2H])c([2H])c([2H])c2-3)c1[2H] ; CC1(C)c2ccccc2-c2cccc(-c3cccc(N(c4ccc(-c5ccc(-c6ccc(-c7ccccc7)cc6)cc5)cc4)c4ccc5c(c4)C4(c6ccccc6-5)C5CC6CC(C5)CC4C6)c3)c21 ; CC1(C)c2ccccc2-c2cccc(-c3cccc(N(c4ccc(-c5ccc(-c6ccccc6-c6ccccc6)cc5)cc4)c4ccc5c(c4)C4(c6ccccc6-5)C5CC6CC(C5)CC4C6)c3)c21]

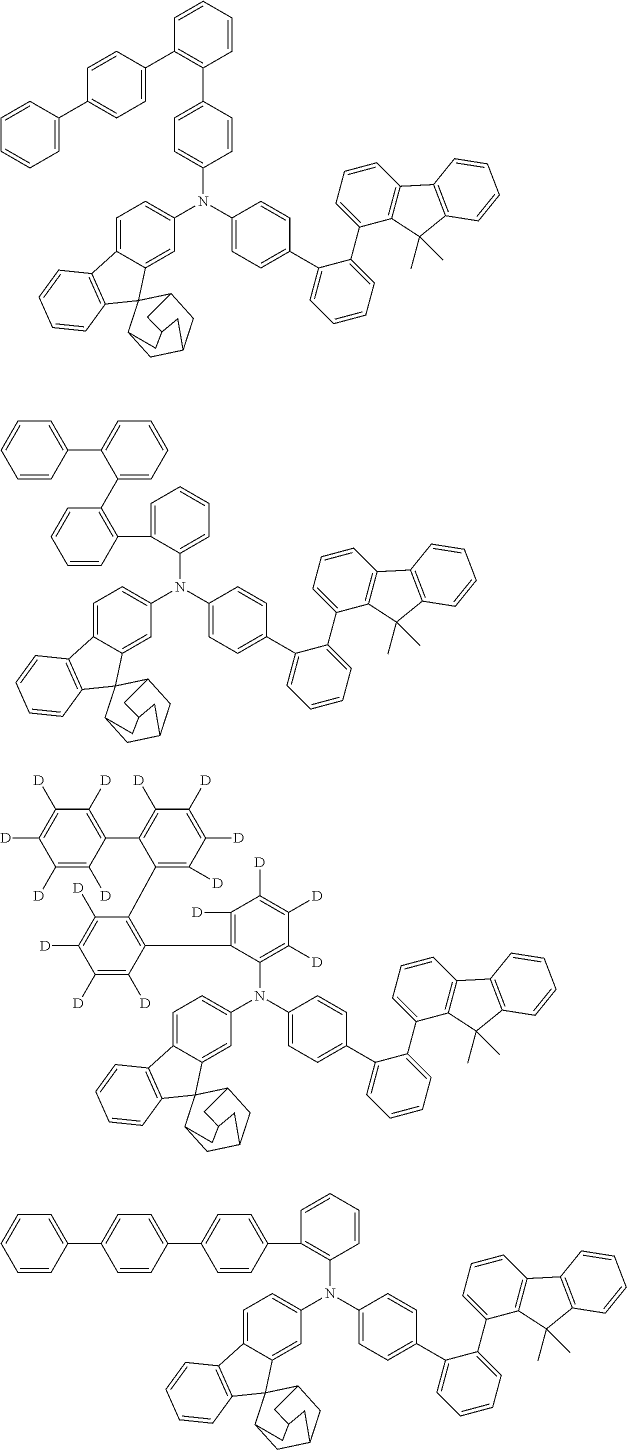

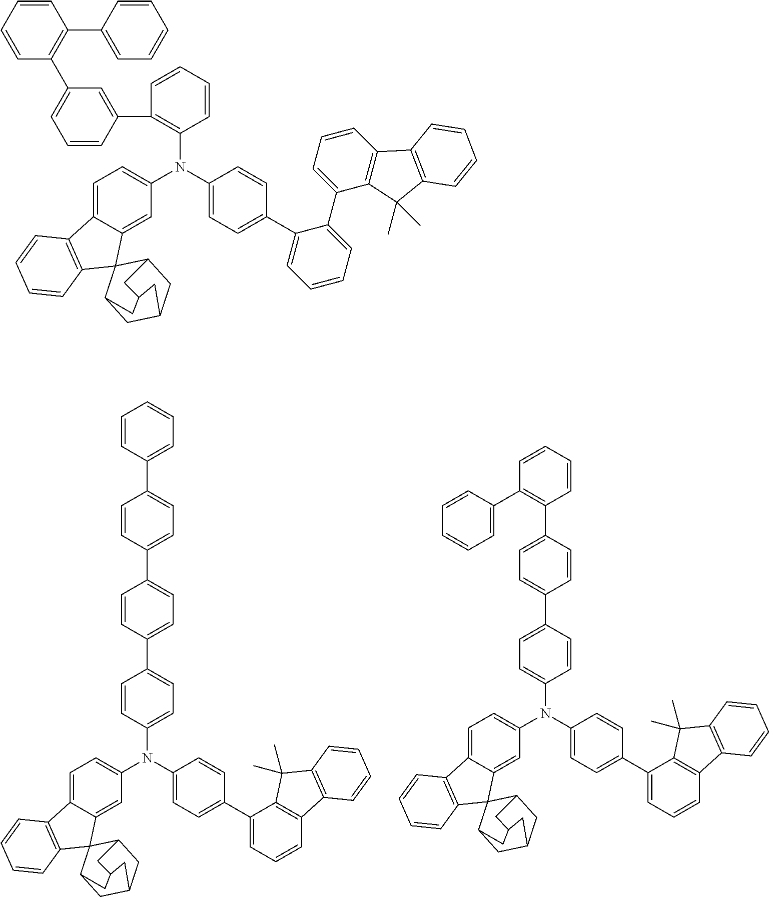

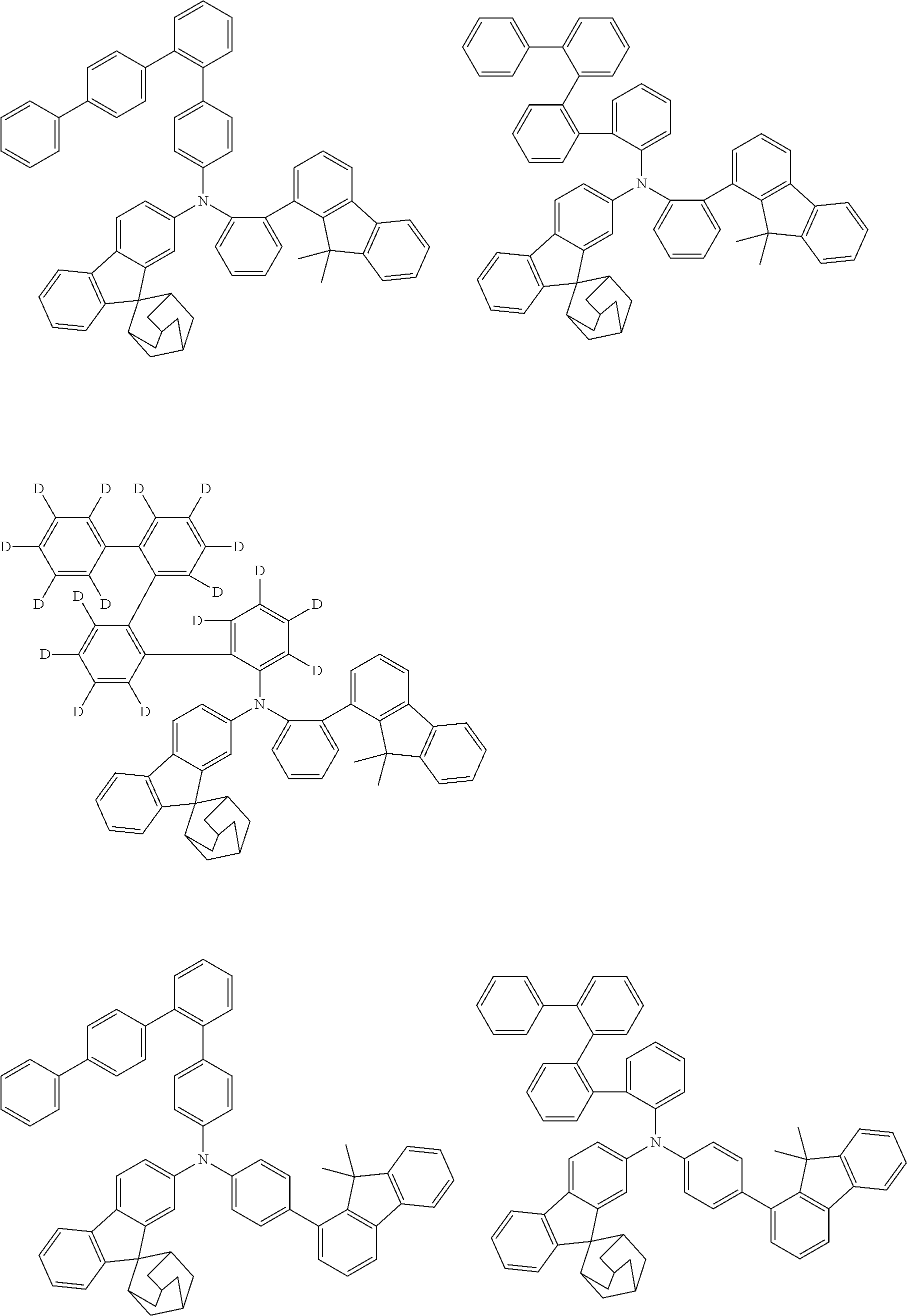

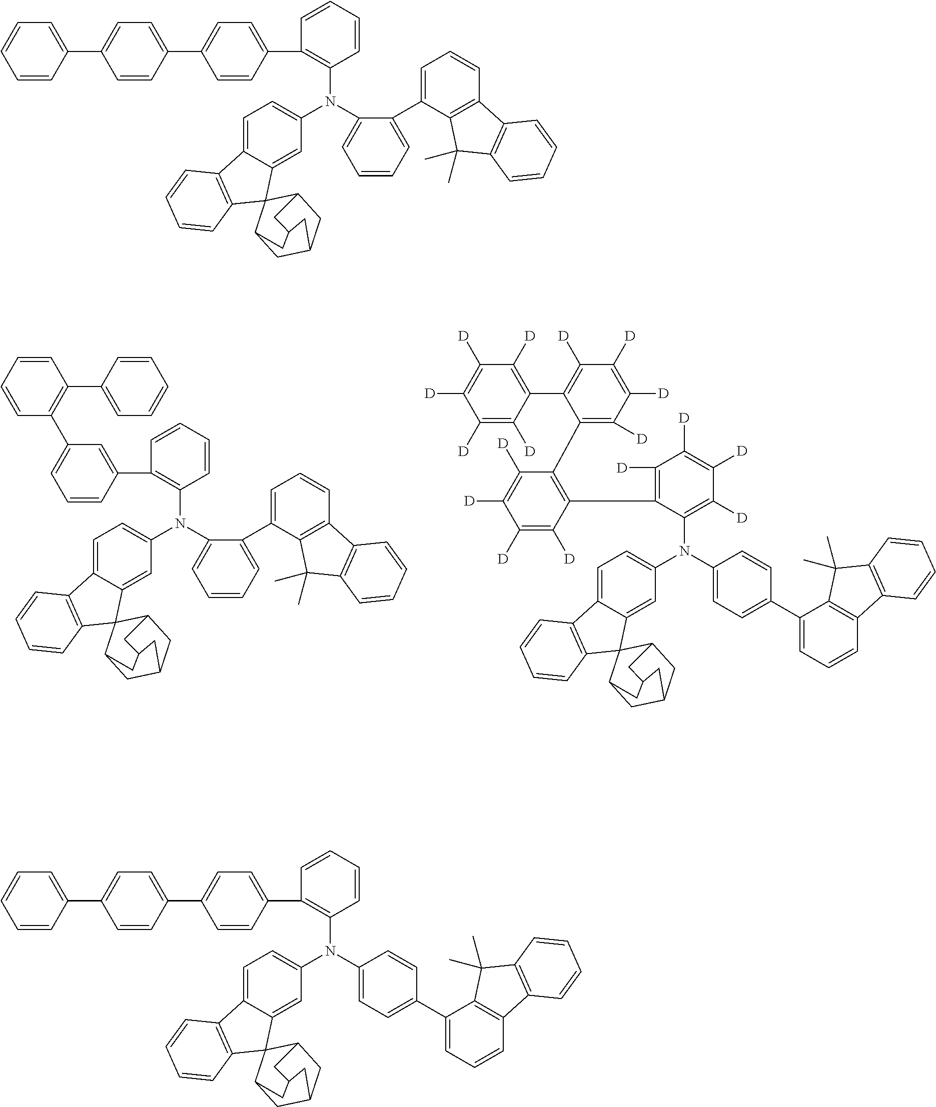

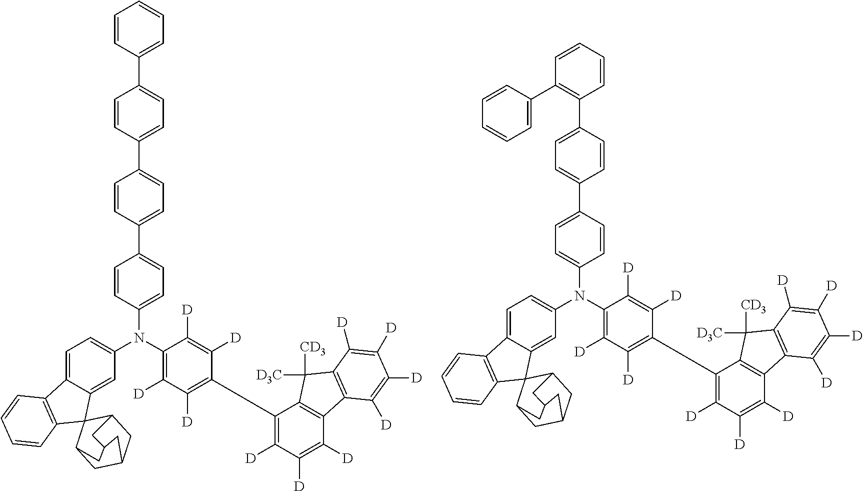

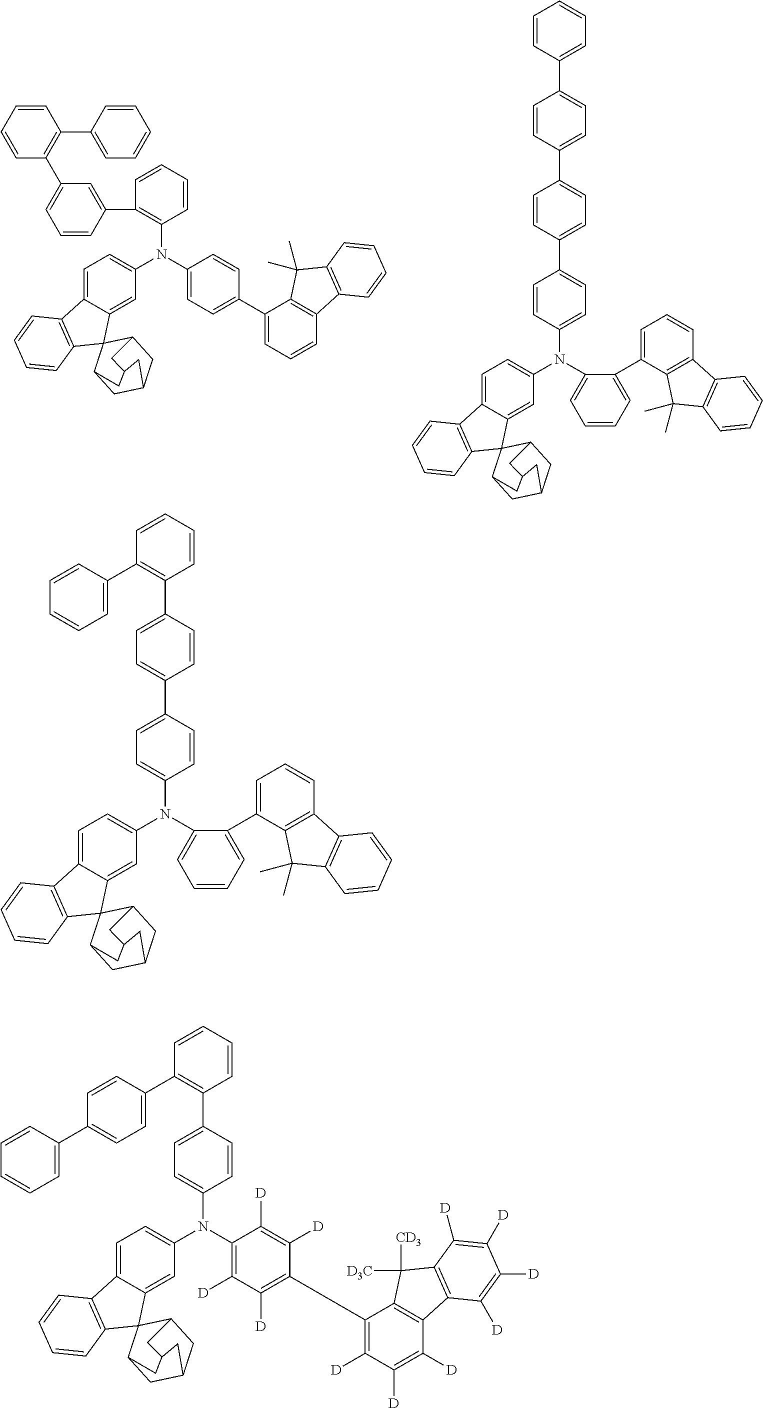

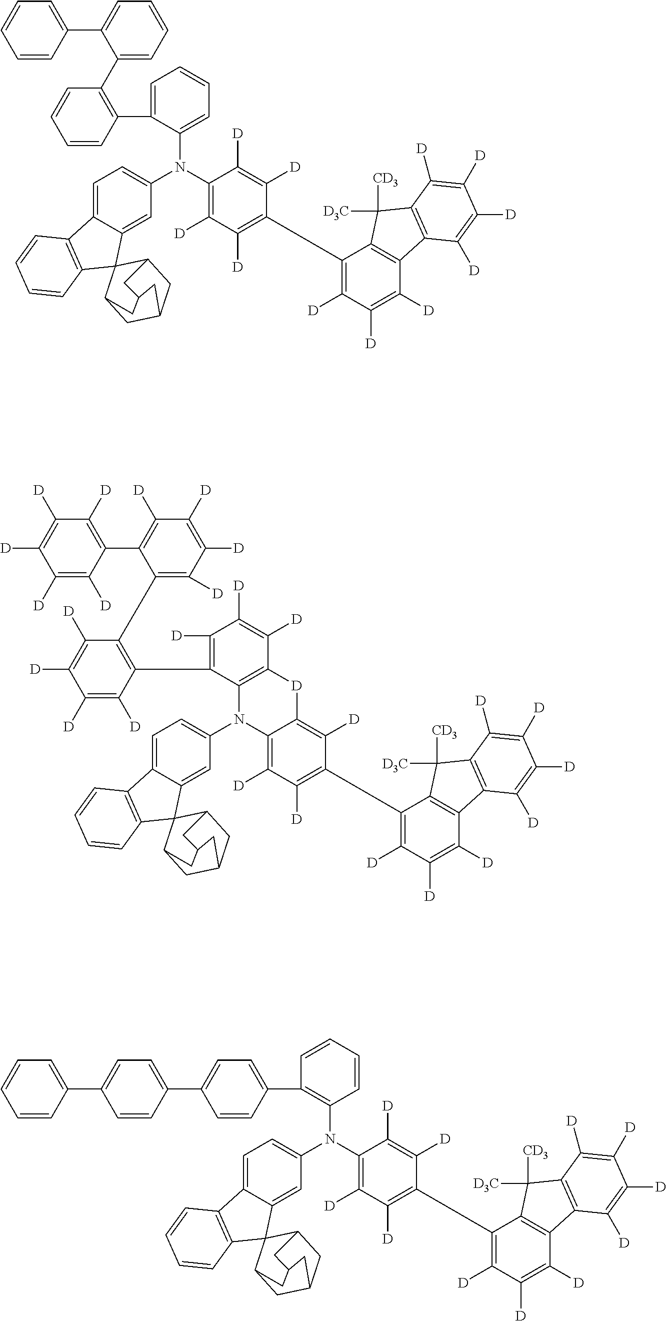

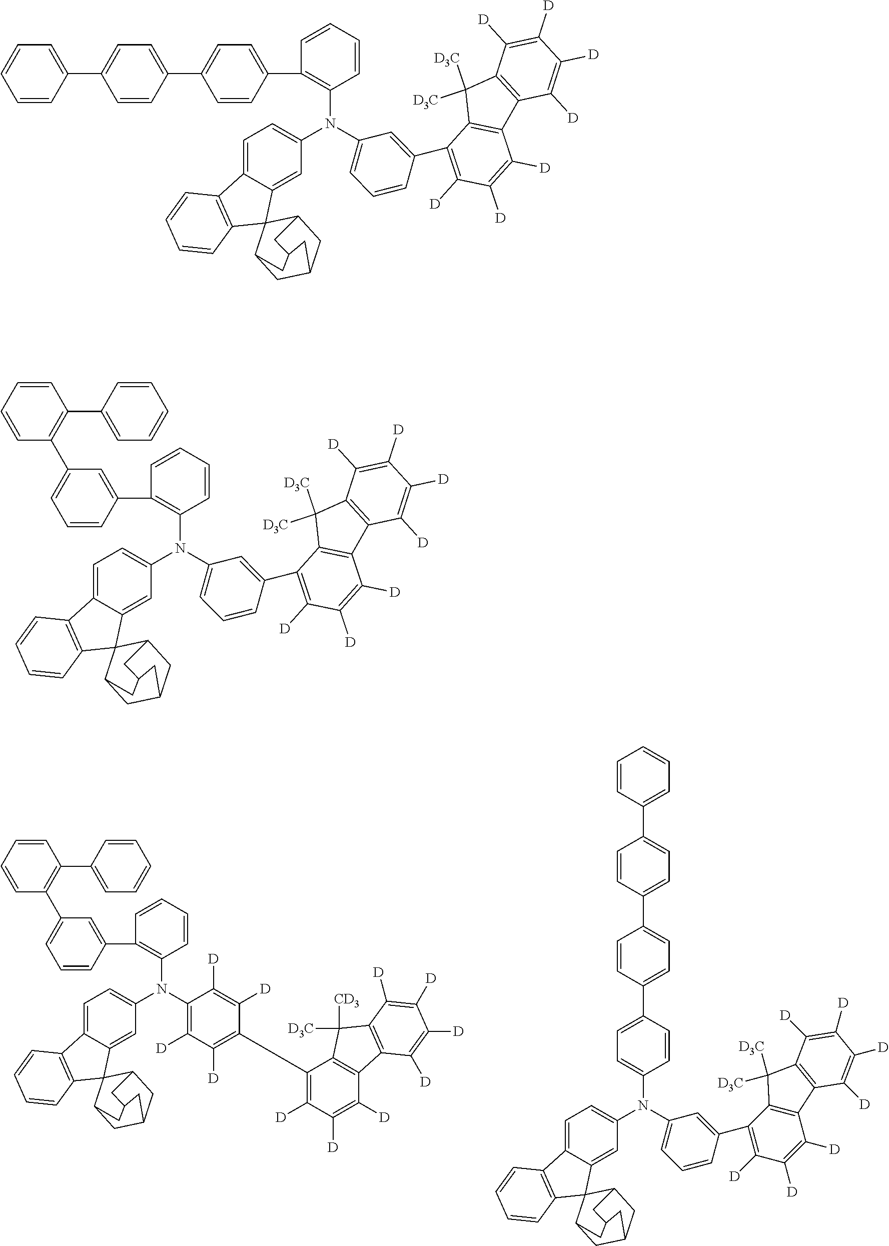

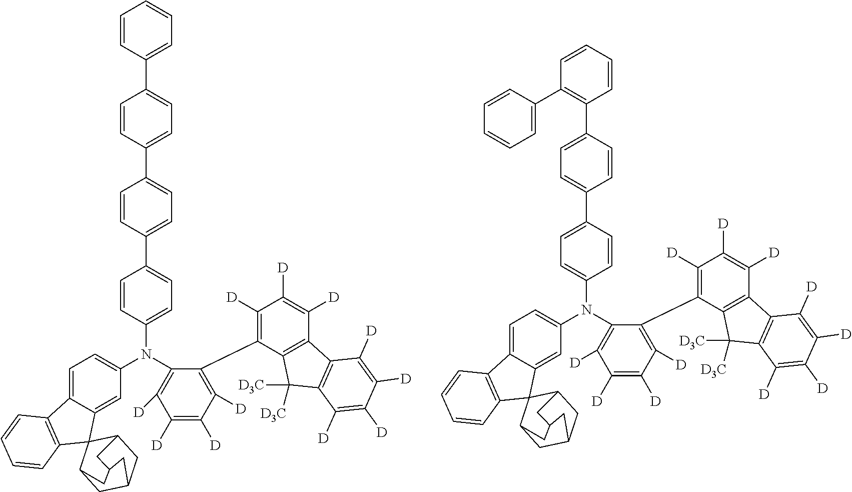

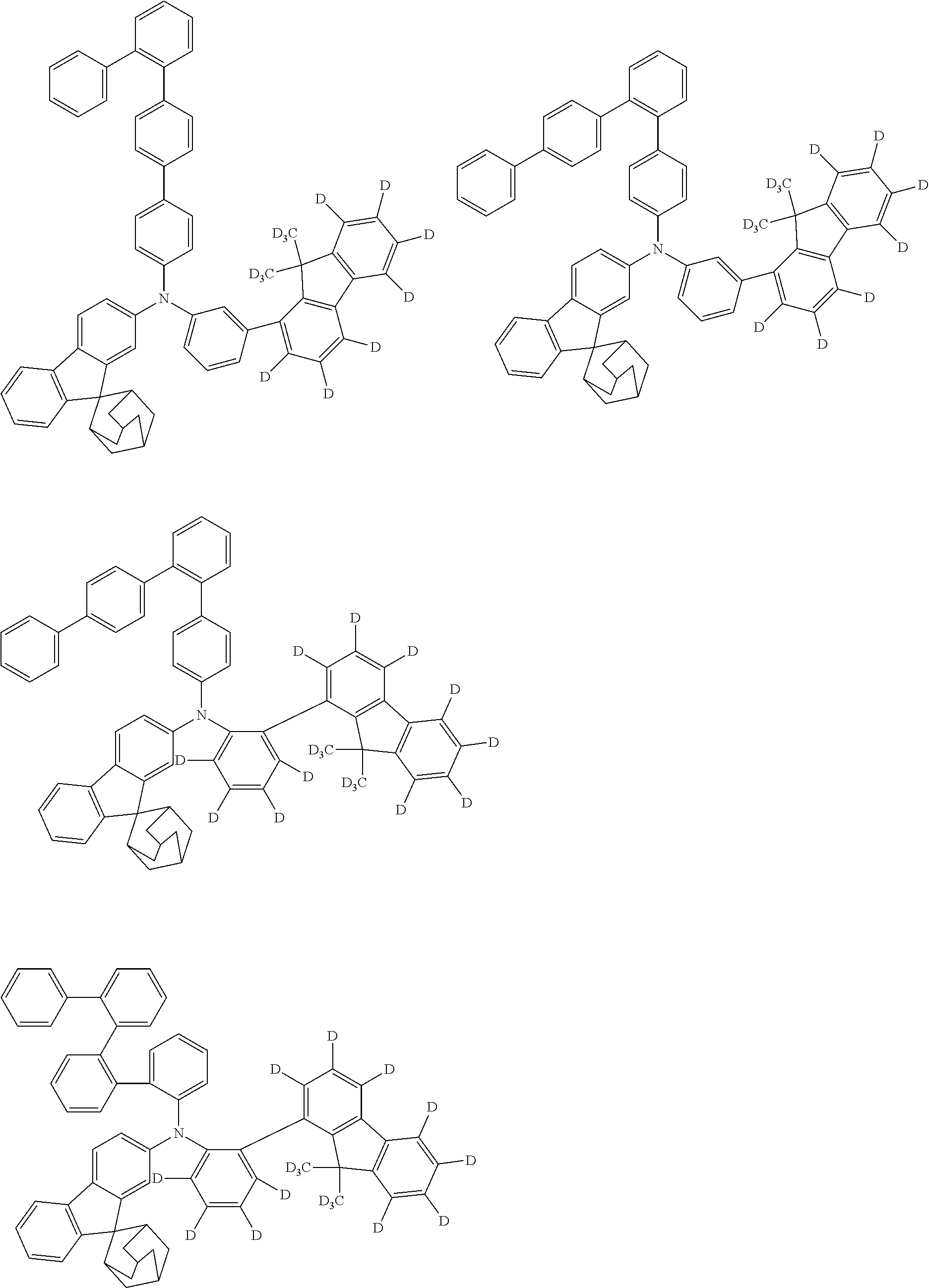

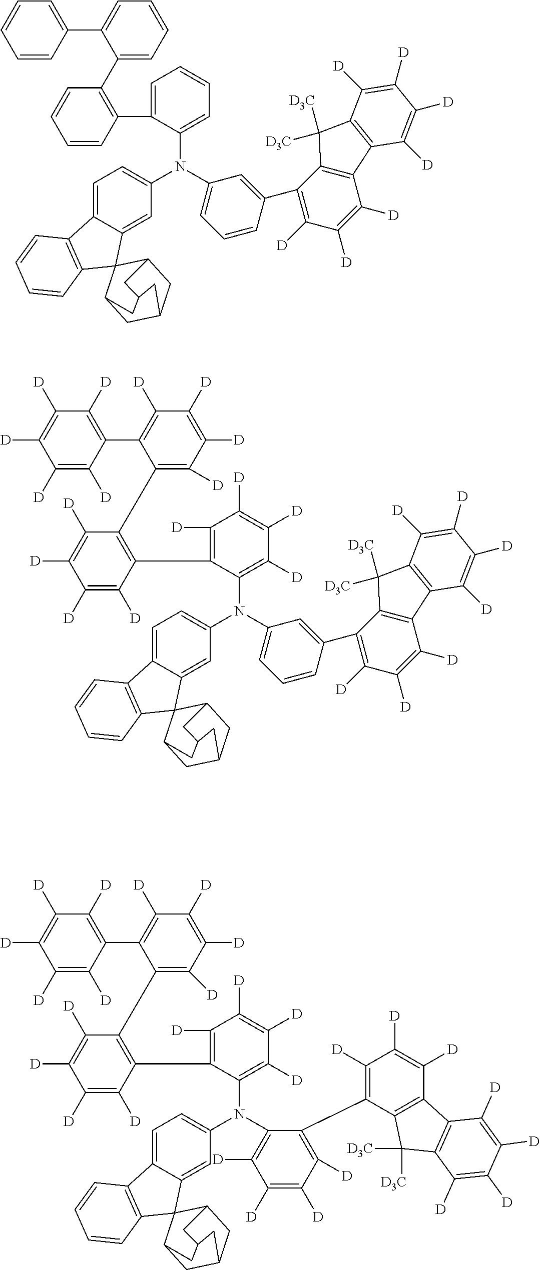

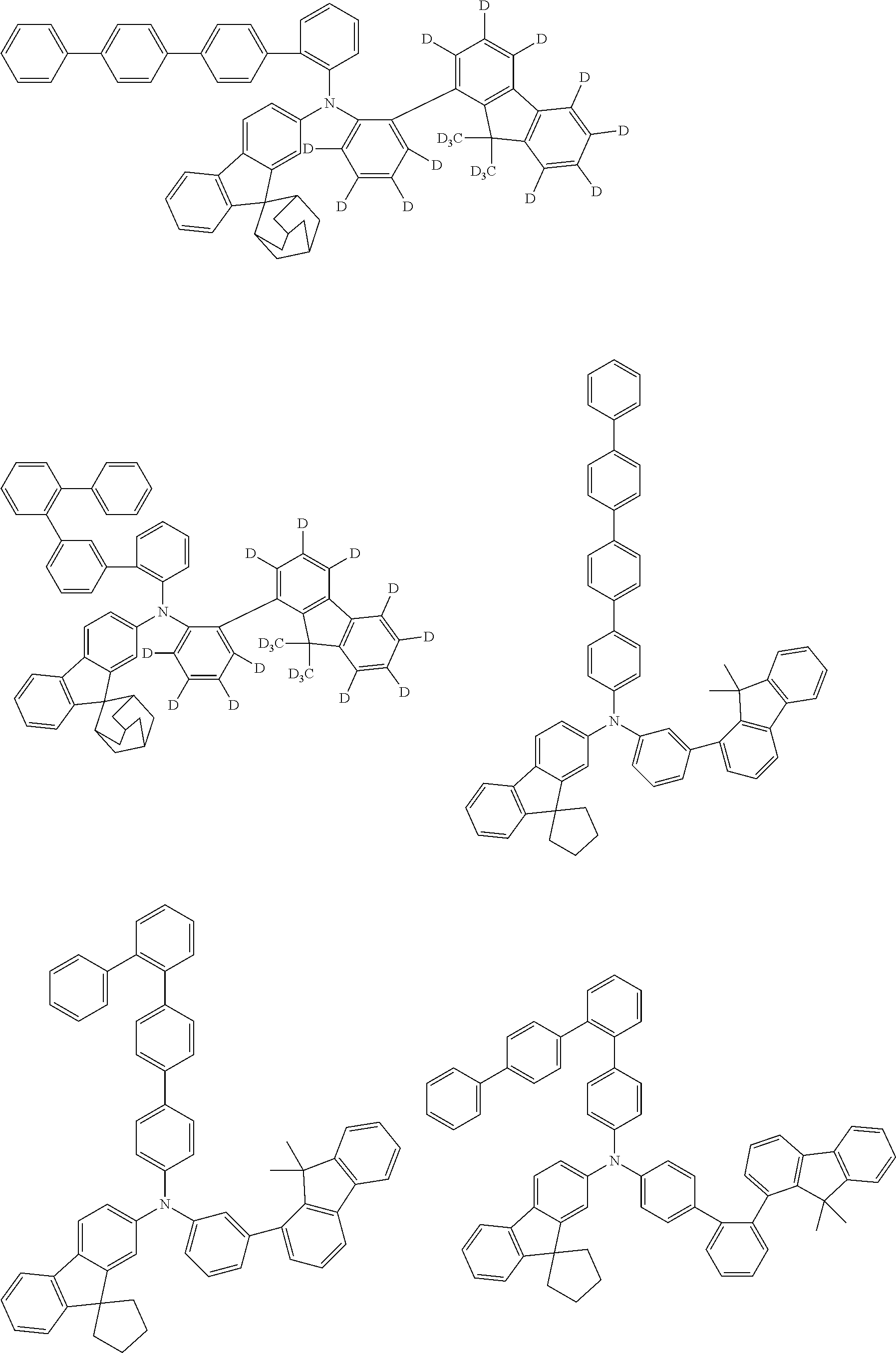

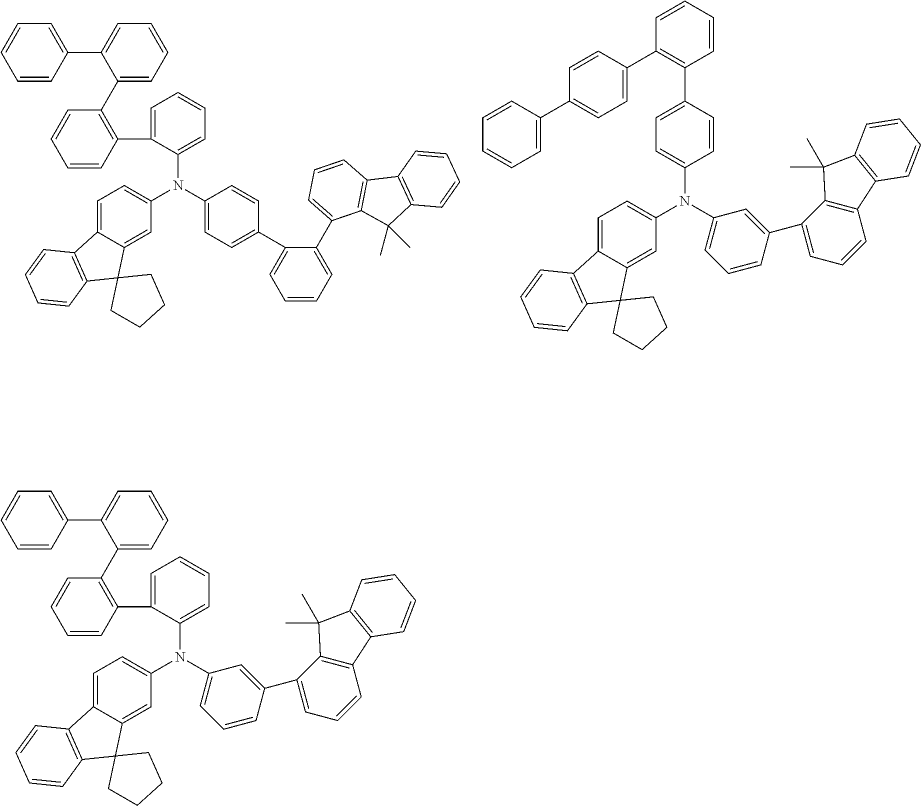

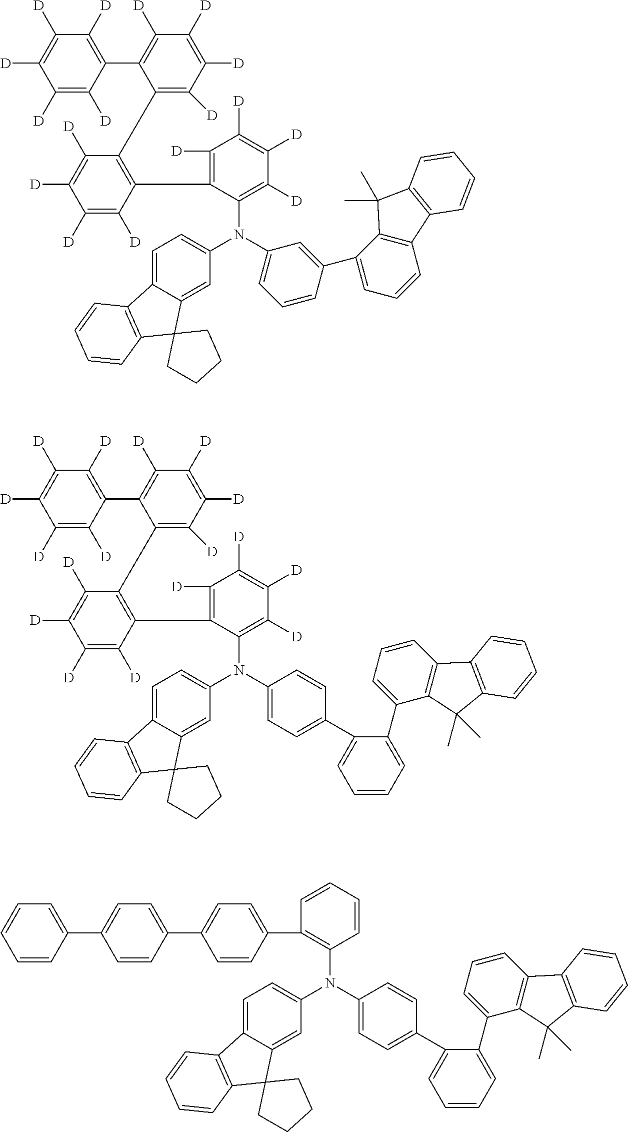

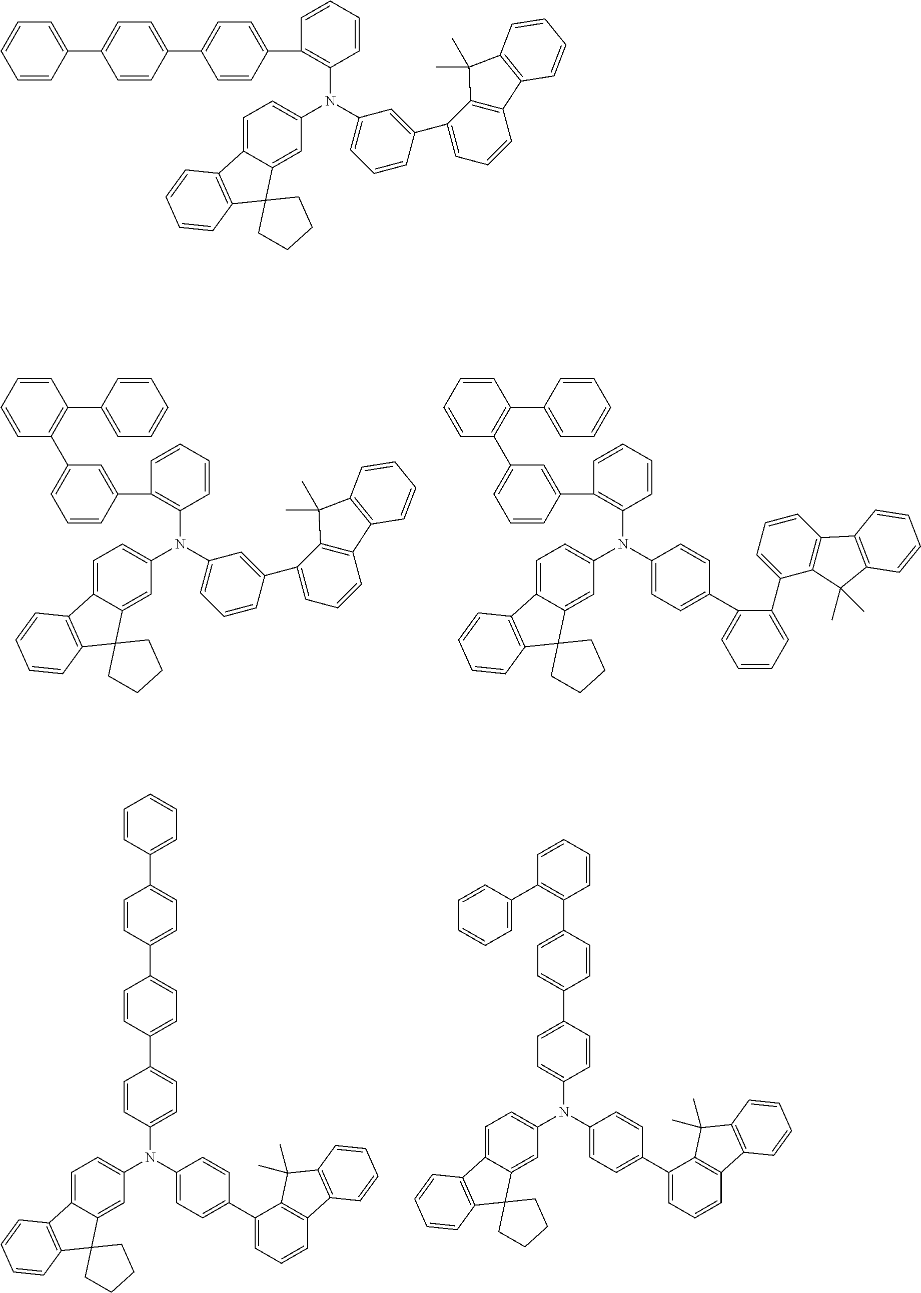

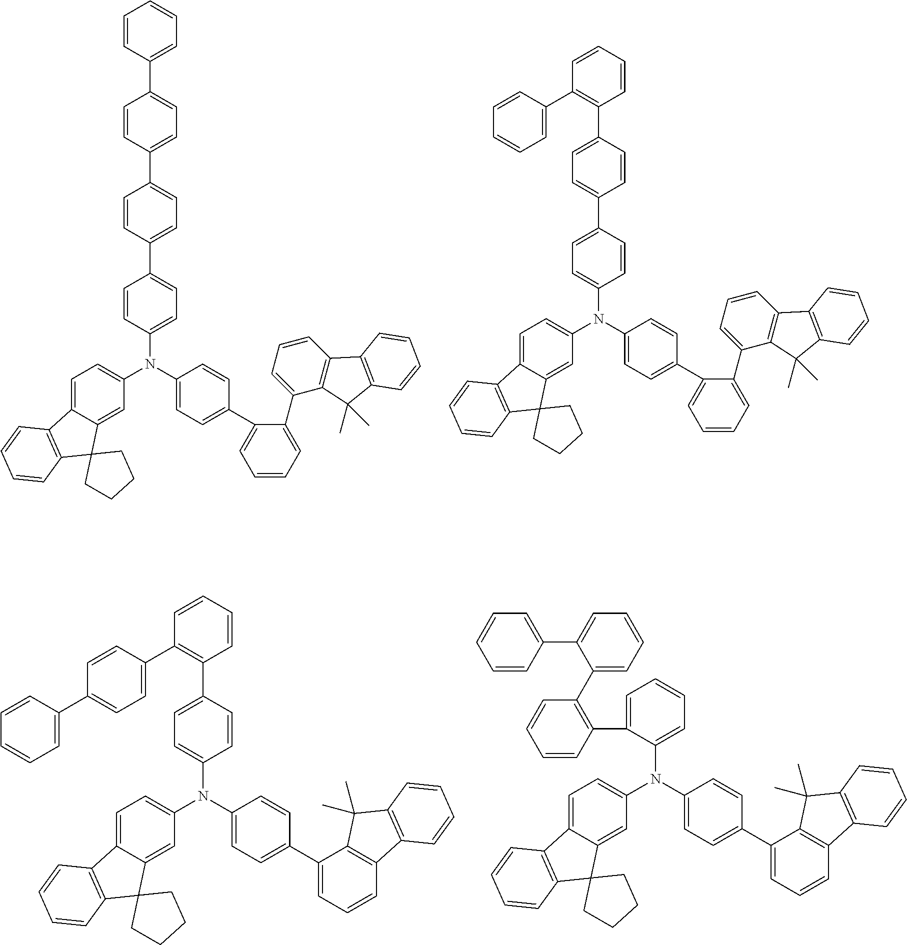 [SMILES: CC1(C)c2ccccc2-c2cccc(-c3ccccc3-c3ccc(N(c4ccc(-c5ccc(-c6ccc(-c7ccccc7)cc6)cc5)cc4)c4ccc5c(c4)C4(CCCC4)c4ccccc4-5)cc3)c21 ; CC1(C)c2ccccc2-c2cccc(-c3ccccc3-c3ccc(N(c4ccc(-c5ccc(-c6ccccc6-c6ccccc6)cc5)cc4)c4ccc5c(c4)C4(CCCC4)c4ccccc4-5)cc3)c21 ; CC1(C)c2ccccc2-c2cccc(-c3ccc(N(c4ccc(-c5ccccc5-c5ccc(-c6ccccc6)cc5)cc4)c4ccc5c(c4)C4(CCCC4)c4ccccc4-5)cc3)c21 ; CC1(C)c2ccccc2-c2cccc(-c3ccc(N(c4ccc5c(c4)C4(CCCC4)c4ccccc4-5)c4ccccc4-c4ccccc4-c4ccccc4-c4ccccc4)cc3)c21]

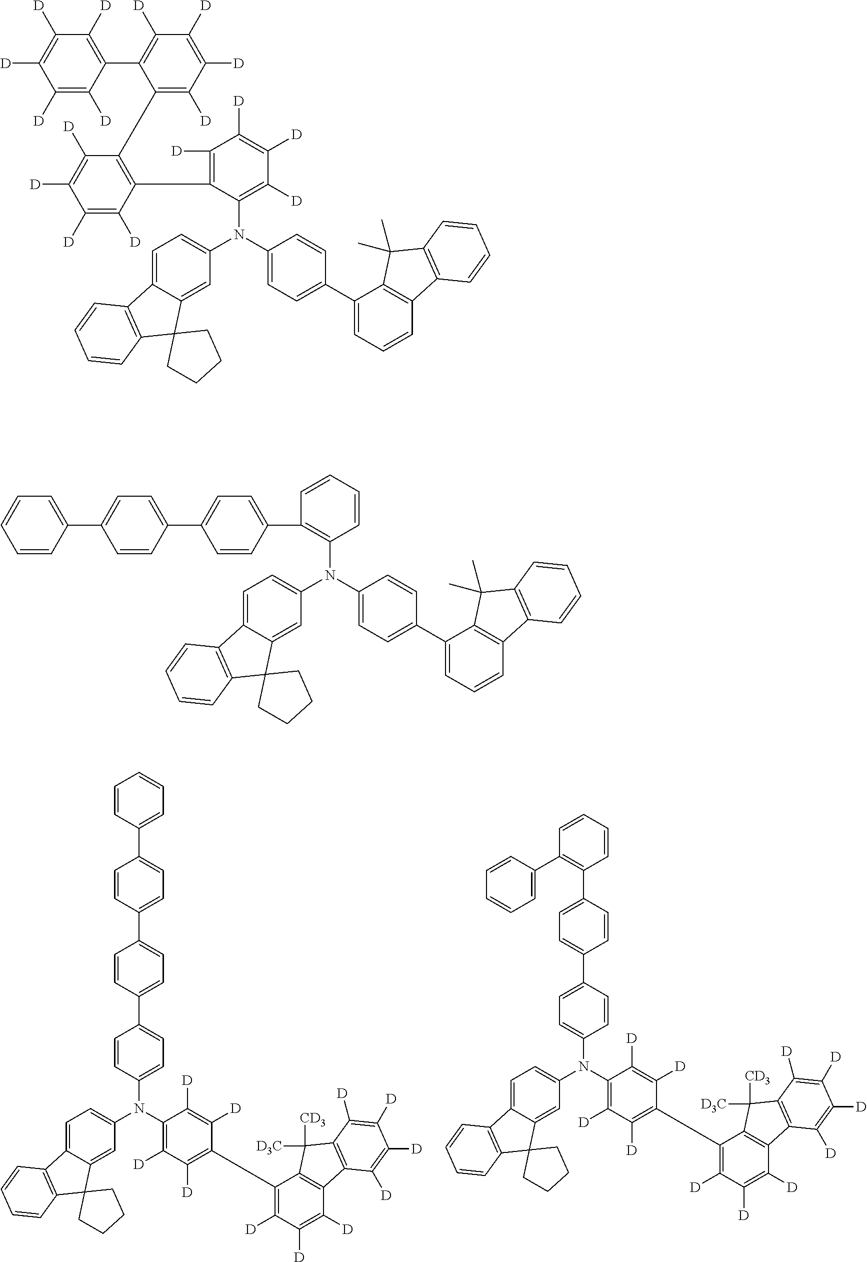

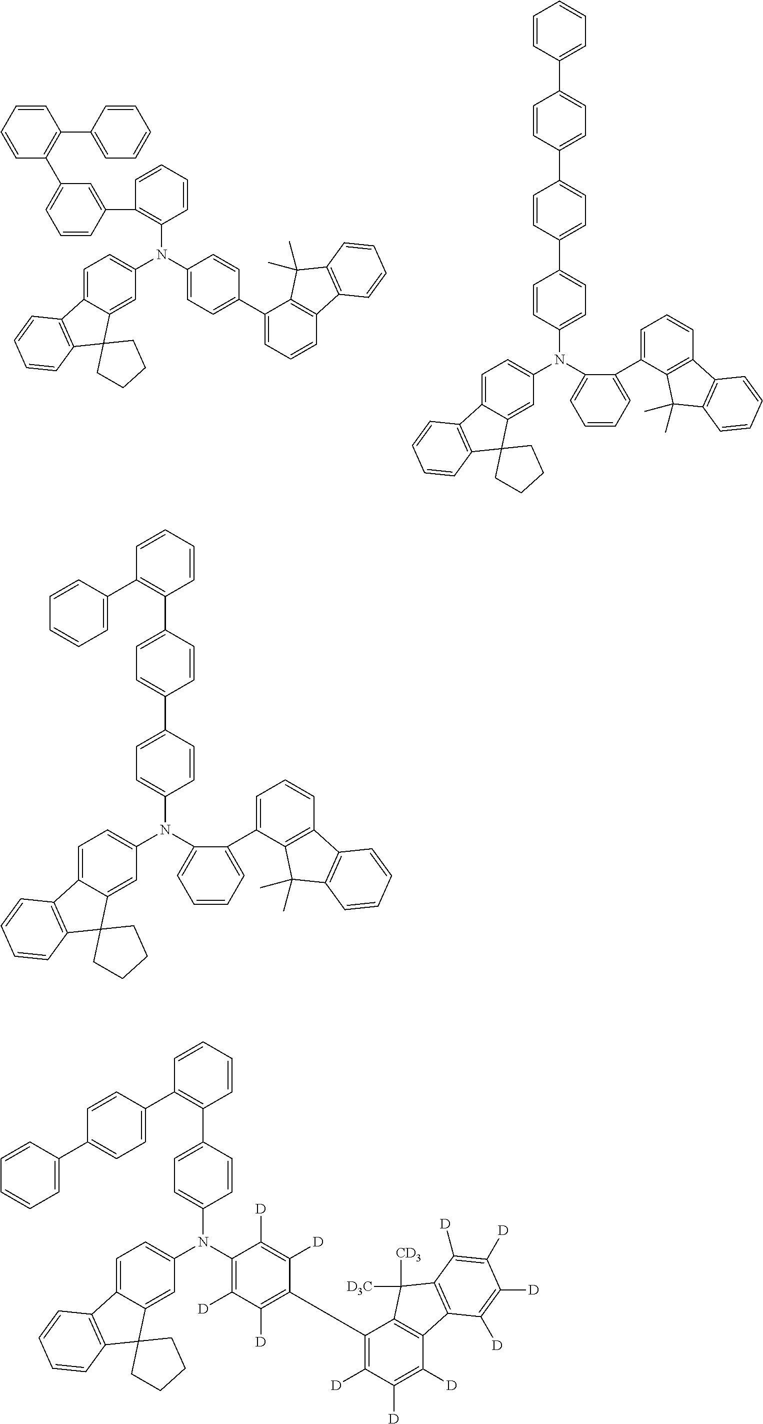

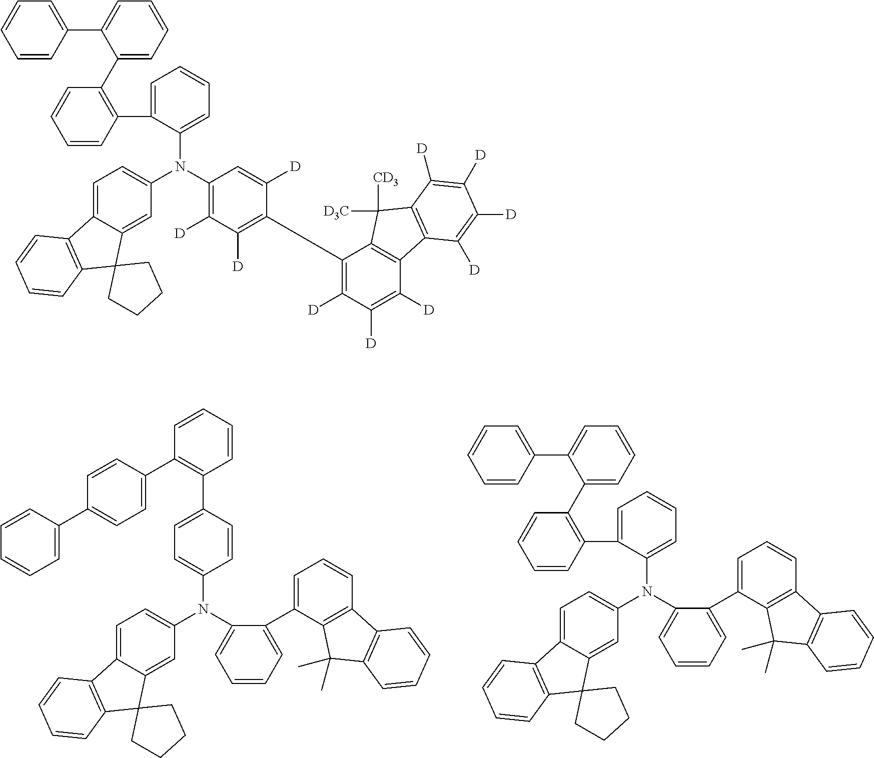 [SMILES: [2H]c1cc(N(c2ccc3c(c2)C2(CCCC2)c2ccccc2-3)c2ccccc2-c2ccccc2-c2ccccc2-c2ccccc2)c([2H])c([2H])c1-c1c([2H])c([2H])c([2H])c2c1C(C([2H])([2H])[2H])(C([2H])([2H])[2H])c1c([2H])c([2H])c([2H])c([2H])c1-2 ; CC1(C)c2ccccc2-c2cccc(-c3ccccc3N(c3ccc(-c4ccccc4-c4ccc(-c5ccccc5)cc4)cc3)c3ccc4c(c3)C3(CCCC3)c3ccccc3-4)c21 ; CC1(C)c2ccccc2-c2cccc(-c3ccccc3N(c3ccc4c(c3)C3(CCCC3)c3ccccc3-4)c3ccccc3-c3ccccc3-c3ccccc3-c3ccccc3)c21]

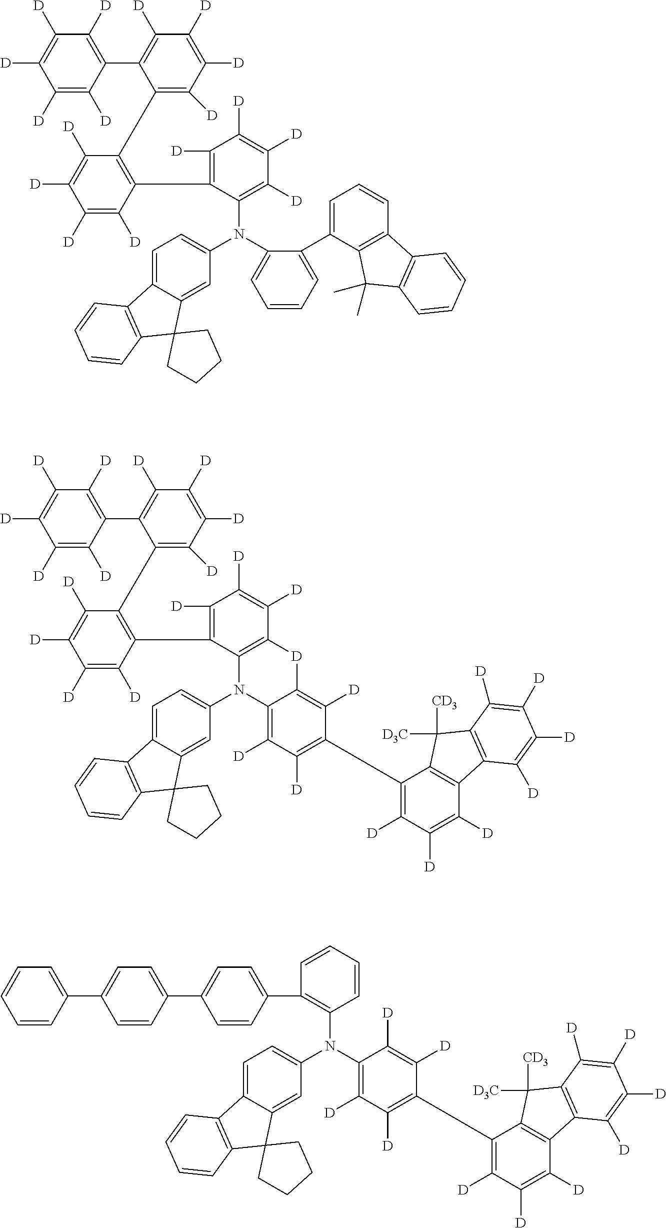

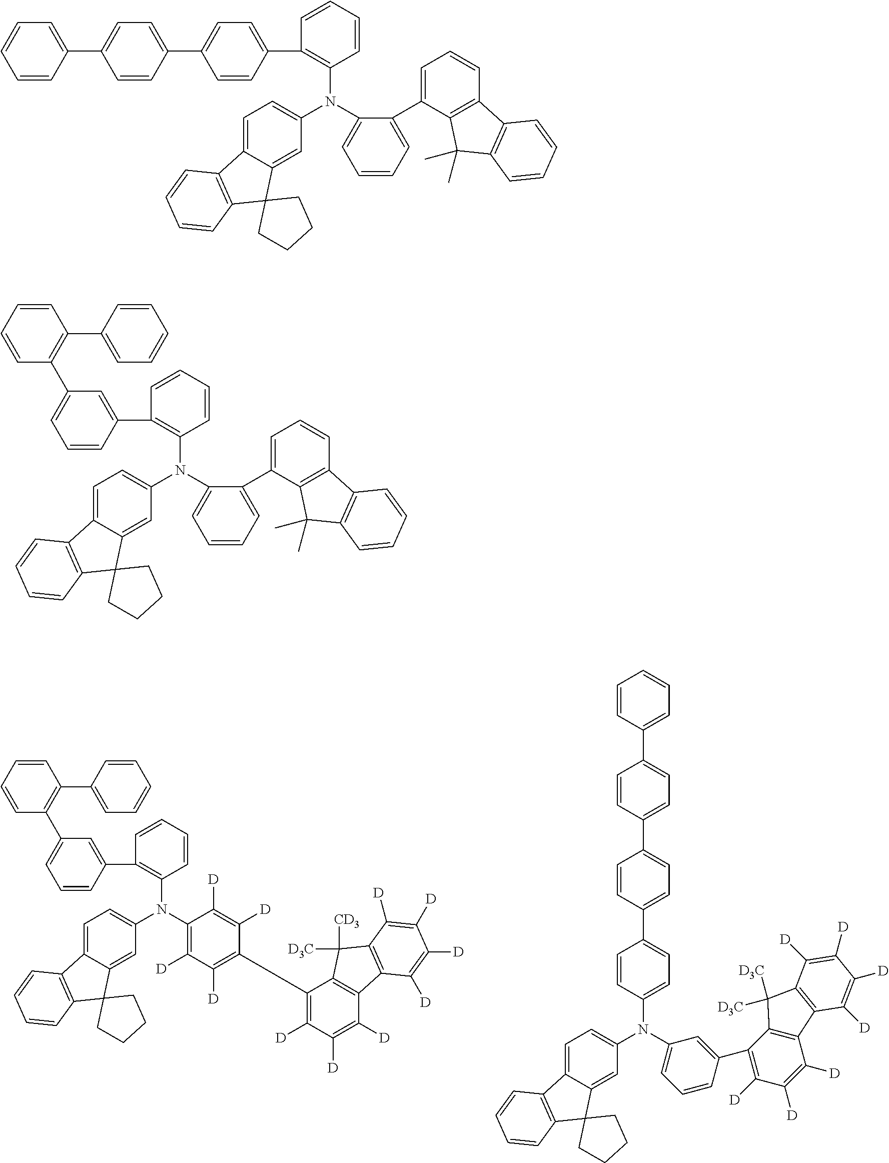

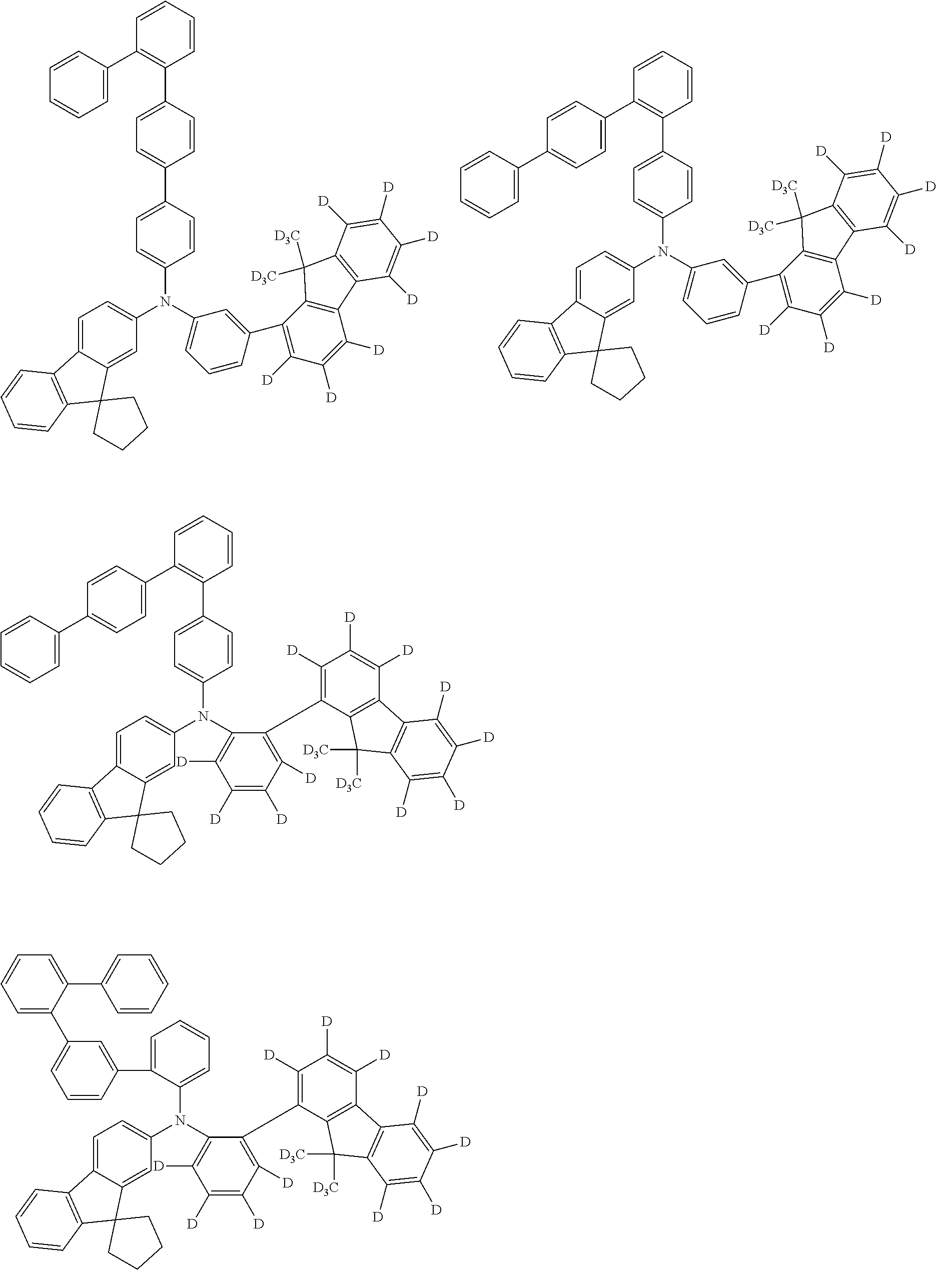

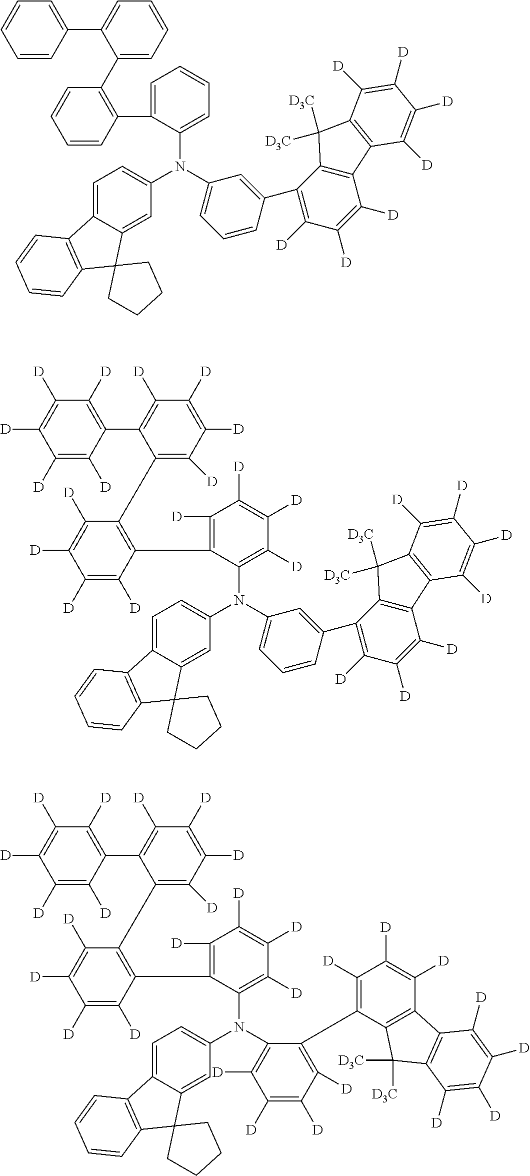

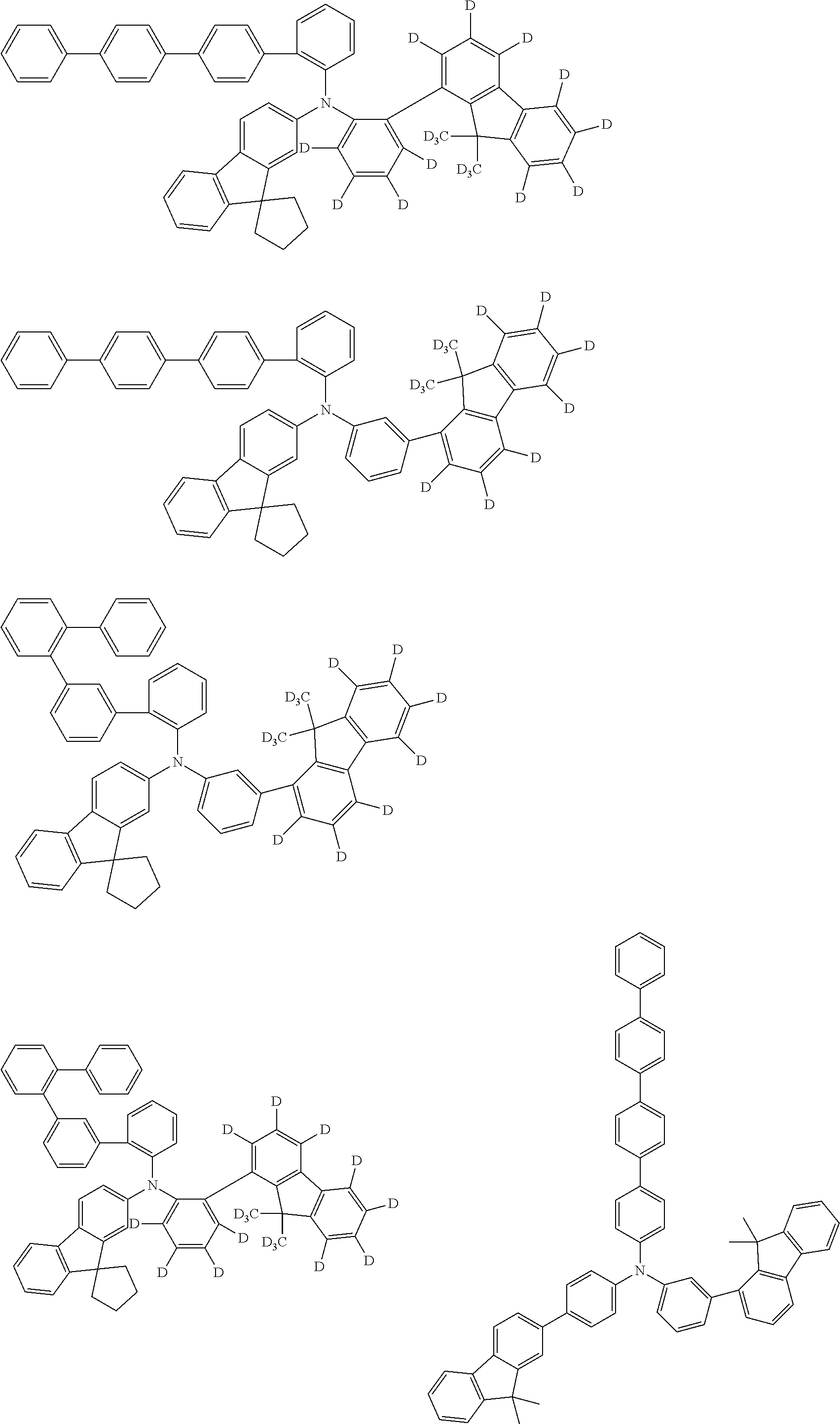

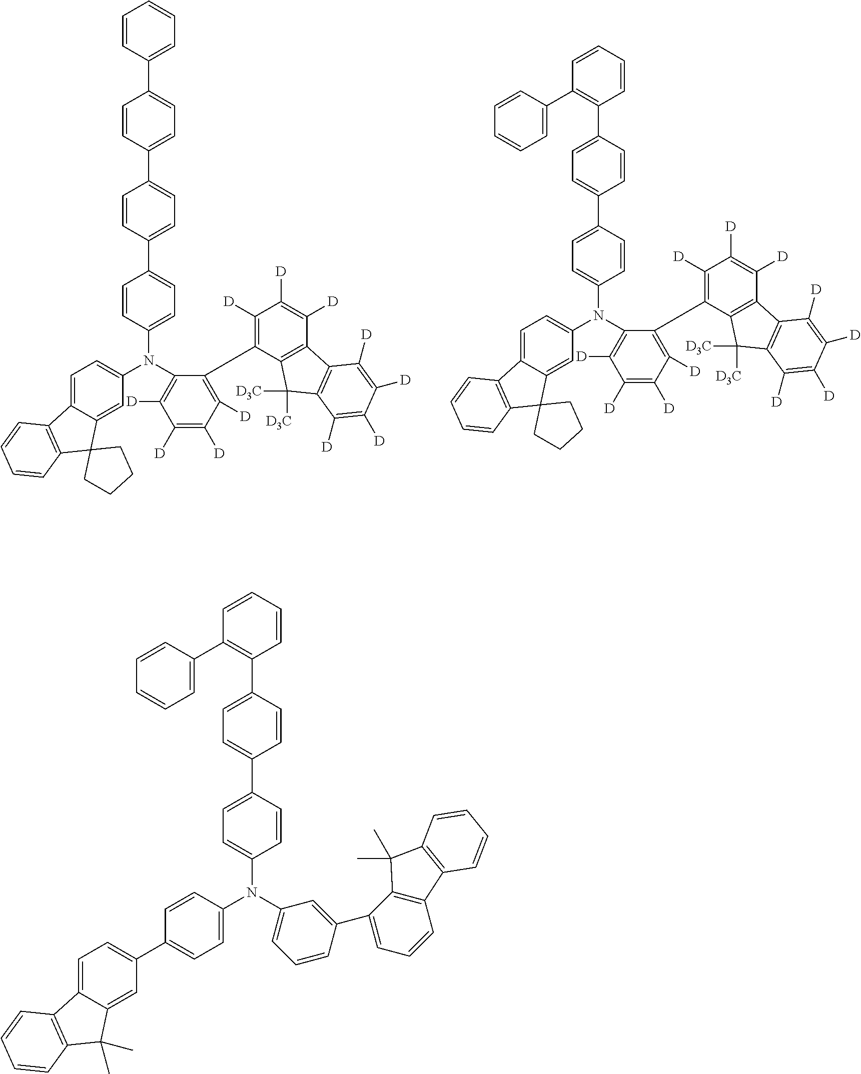

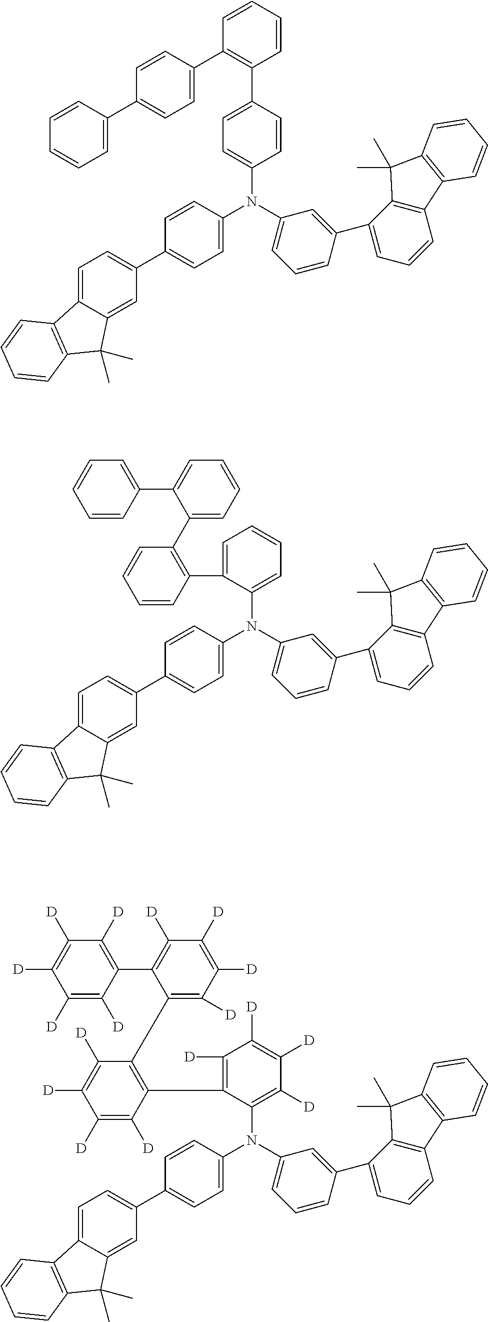 [SMILES: CC1(C)c2ccccc2-c2ccc(-c3ccc(N(c4ccc(-c5ccccc5-c5ccc(-c6ccccc6)cc5)cc4)c4cccc(-c5cccc6c5C(C)(C)c5ccccc5-6)c4)cc3)cc21 ; CC1(C)c2ccccc2-c2ccc(-c3ccc(N(c4cccc(-c5cccc6c5C(C)(C)c5ccccc5-6)c4)c4ccccc4-c4ccccc4-c4ccccc4-c4ccccc4)cc3)cc21 ; [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2ccc(-c3ccc4c(c3)C(C)(C)c3ccccc3-4)cc2)c2cccc(-c3cccc4c3C(C)(C)c3ccccc3-4)c2)c([2H])c1[2H]]

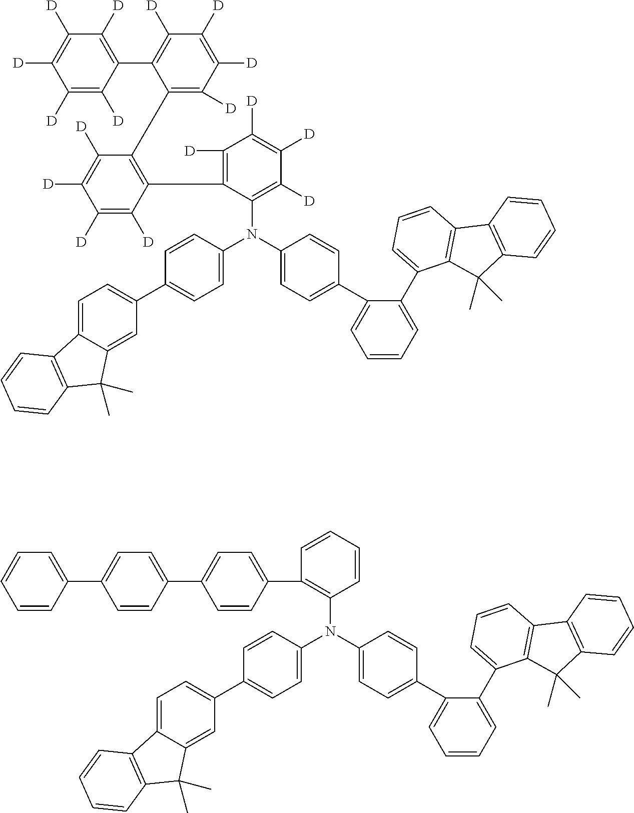 [SMILES: [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2ccc(-c3ccc4c(c3)C(C)(C)c3ccccc3-4)cc2)c2ccc(-c3ccccc3-c3cccc4c3C(C)(C)c3ccccc3-4)cc2)c([2H])c1[2H] ; CC1(C)c2ccccc2-c2ccc(-c3ccc(N(c4ccc(-c5ccccc5-c5cccc6c5C(C)(C)c5ccccc5-6)cc4)c4ccccc4-c4ccc(-c5ccc(-c6ccccc6)cc5)cc4)cc3)cc21]

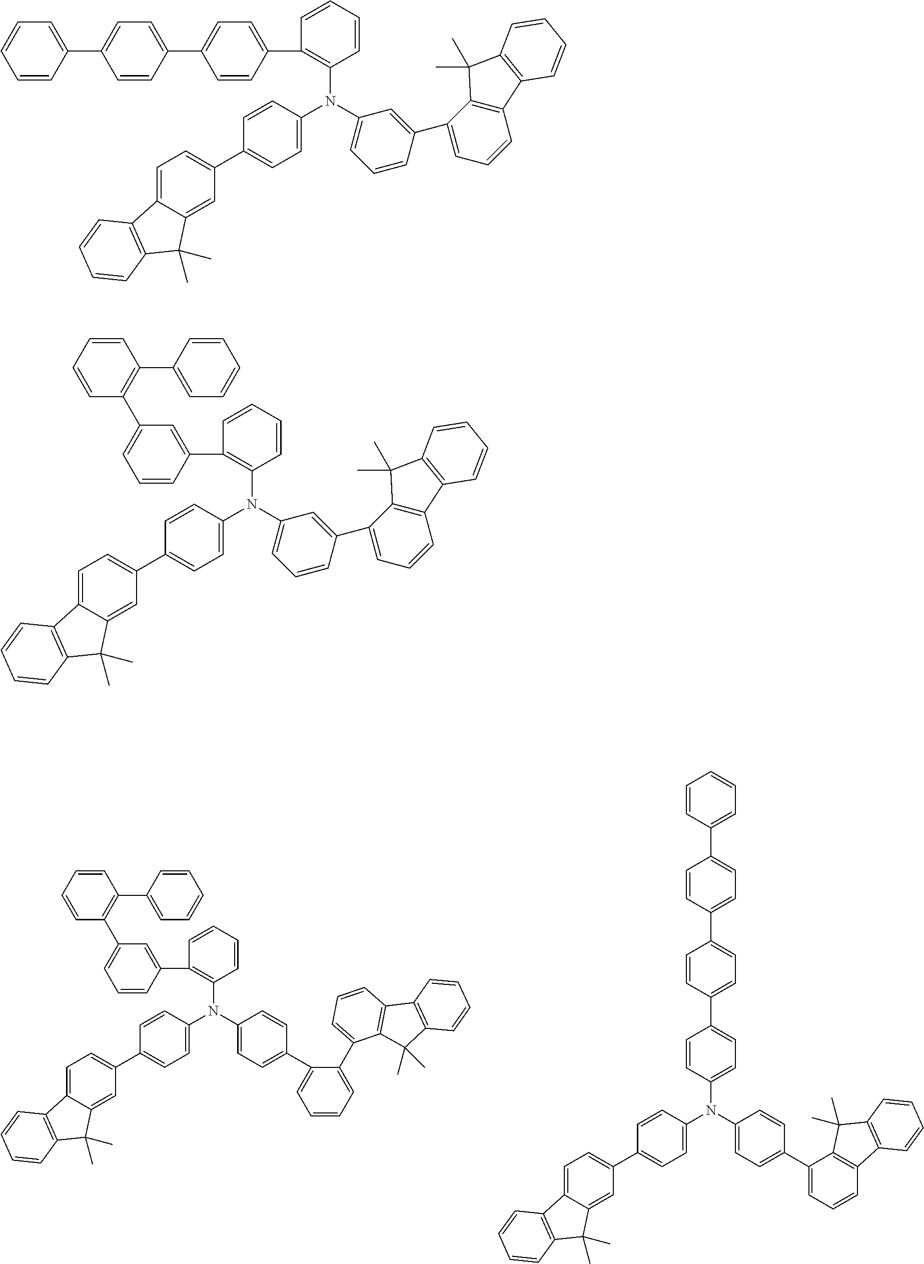 [SMILES: CC1(C)c2ccccc2-c2ccc(-c3ccc(N(c4cccc(-c5cccc6c5C(C)(C)c5ccccc5-6)c4)c4ccccc4-c4ccc(-c5ccc(-c6ccccc6)cc5)cc4)cc3)cc21 ; CC1(C)c2ccccc2-c2ccc(-c3ccc(N(c4cccc(-c5cccc6c5C(C)(C)c5ccccc5-6)c4)c4ccccc4-c4cccc(-c5ccccc5-c5ccccc5)c4)cc3)cc21 ; CC1(C)c2ccccc2-c2ccc(-c3ccc(N(c4ccc(-c5ccccc5-c5cccc6c5C(C)(C)c5ccccc5-6)cc4)c4ccccc4-c4cccc(-c5ccccc5-c5ccccc5)c4)cc3)cc21 ; CC1(C)c2ccccc2-c2ccc(-c3ccc(N(c4ccc(-c5ccc(-c6ccc(-c7ccccc7)cc6)cc5)cc4)c4ccc(-c5cccc6c5C(C)(C)c5ccccc5-6)cc4)cc3)cc21]

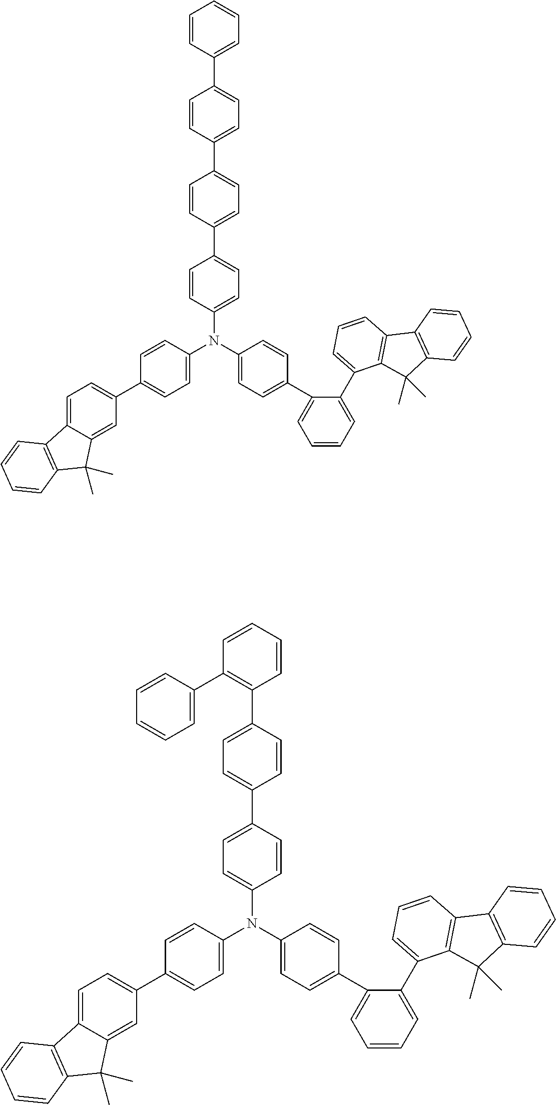 [SMILES: CC1(C)c2ccccc2-c2ccc(-c3ccc(N(c4ccc(-c5ccc(-c6ccc(-c7ccccc7)cc6)cc5)cc4)c4ccc(-c5ccccc5-c5cccc6c5C(C)(C)c5ccccc5-6)cc4)cc3)cc21 ; CC1(C)c2ccccc2-c2ccc(-c3ccc(N(c4ccc(-c5ccc(-c6ccccc6-c6ccccc6)cc5)cc4)c4ccc(-c5ccccc5-c5cccc6c5C(C)(C)c5ccccc5-6)cc4)cc3)cc21]

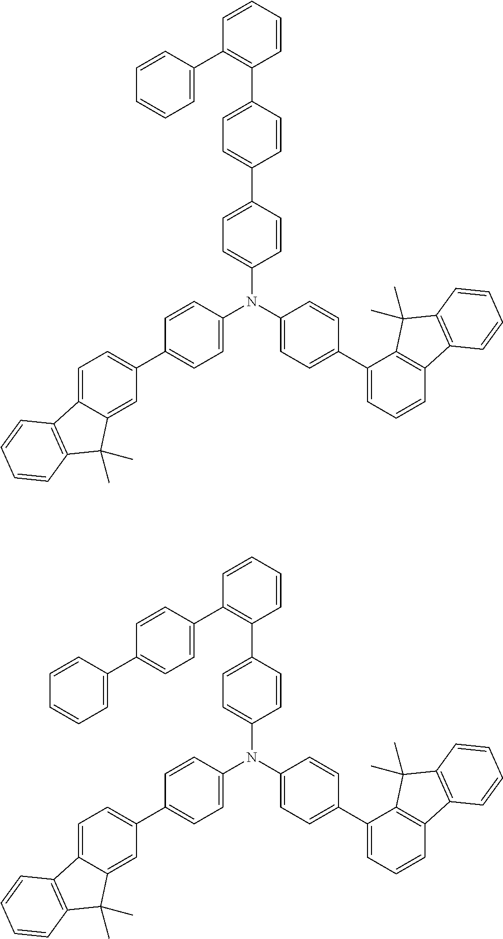 [SMILES: CC1(C)c2ccccc2-c2ccc(-c3ccc(N(c4ccc(-c5ccc(-c6ccccc6-c6ccccc6)cc5)cc4)c4ccc(-c5cccc6c5C(C)(C)c5ccccc5-6)cc4)cc3)cc21 ; CC1(C)c2ccccc2-c2ccc(-c3ccc(N(c4ccc(-c5ccccc5-c5ccc(-c6ccccc6)cc5)cc4)c4ccc(-c5cccc6c5C(C)(C)c5ccccc5-6)cc4)cc3)cc21]

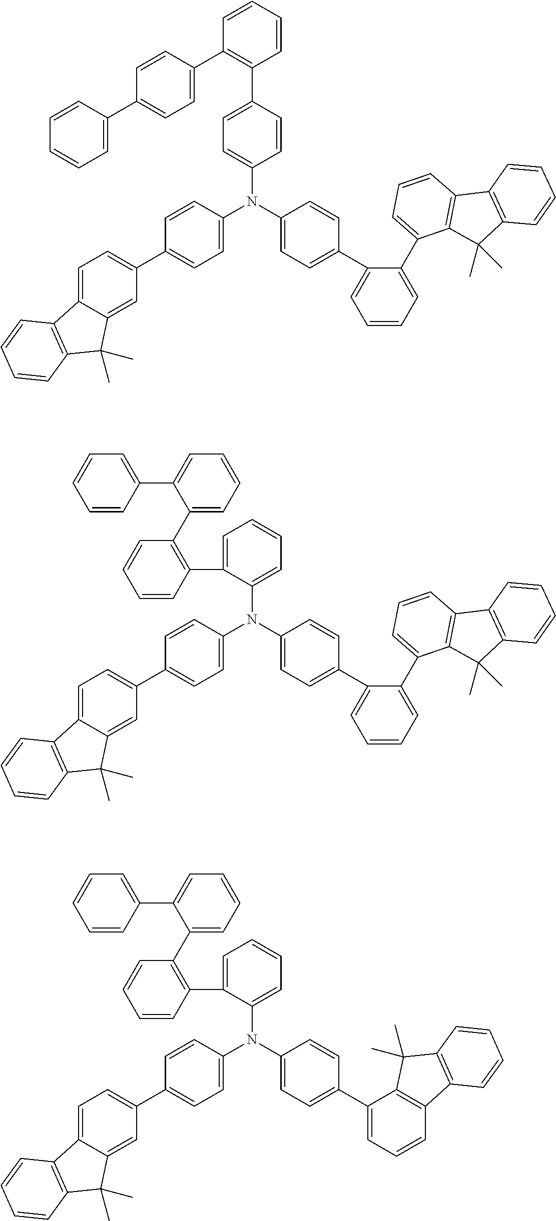 [SMILES: CC1(C)c2ccccc2-c2ccc(-c3ccc(N(c4ccc(-c5ccccc5-c5ccc(-c6ccccc6)cc5)cc4)c4ccc(-c5ccccc5-c5cccc6c5C(C)(C)c5ccccc5-6)cc4)cc3)cc21 ; CC1(C)c2ccccc2-c2ccc(-c3ccc(N(c4ccc(-c5ccccc5-c5cccc6c5C(C)(C)c5ccccc5-6)cc4)c4ccccc4-c4ccccc4-c4ccccc4-c4ccccc4)cc3)cc21 ; CC1(C)c2ccccc2-c2ccc(-c3ccc(N(c4ccc(-c5cccc6c5C(C)(C)c5ccccc5-6)cc4)c4ccccc4-c4ccccc4-c4ccccc4-c4ccccc4)cc3)cc21]

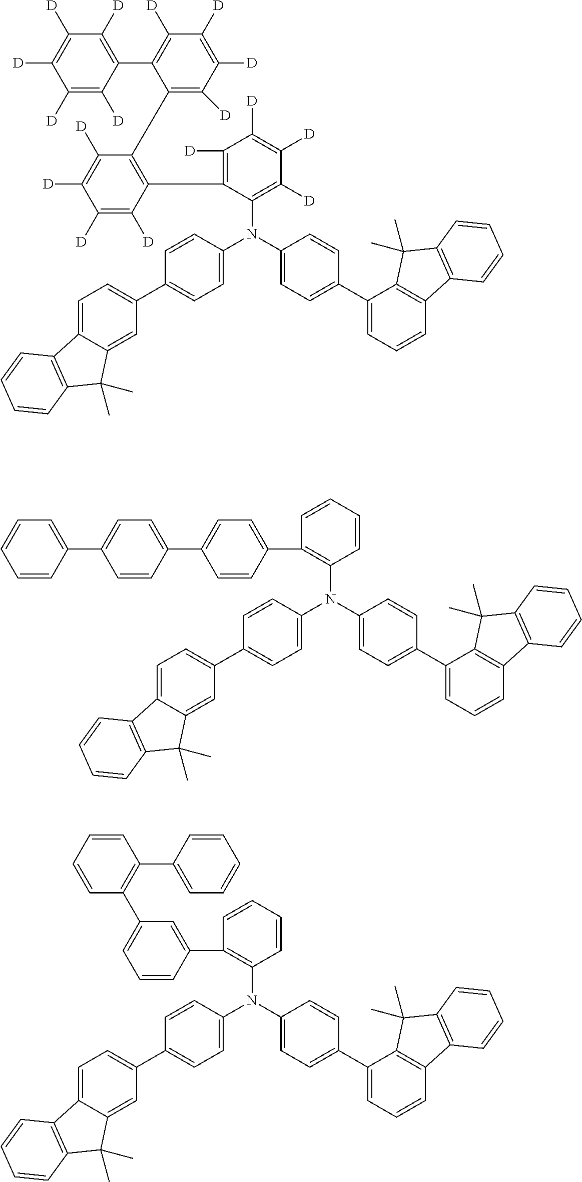 [SMILES: [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2ccc(-c3ccc4c(c3)C(C)(C)c3ccccc3-4)cc2)c2ccc(-c3cccc4c3C(C)(C)c3ccccc3-4)cc2)c([2H])c1[2H] ; CC1(C)c2ccccc2-c2ccc(-c3ccc(N(c4ccc(-c5cccc6c5C(C)(C)c5ccccc5-6)cc4)c4ccccc4-c4ccc(-c5ccc(-c6ccccc6)cc5)cc4)cc3)cc21 ; CC1(C)c2ccccc2-c2ccc(-c3ccc(N(c4ccc(-c5cccc6c5C(C)(C)c5ccccc5-6)cc4)c4ccccc4-c4cccc(-c5ccccc5-c5ccccc5)c4)cc3)cc21]

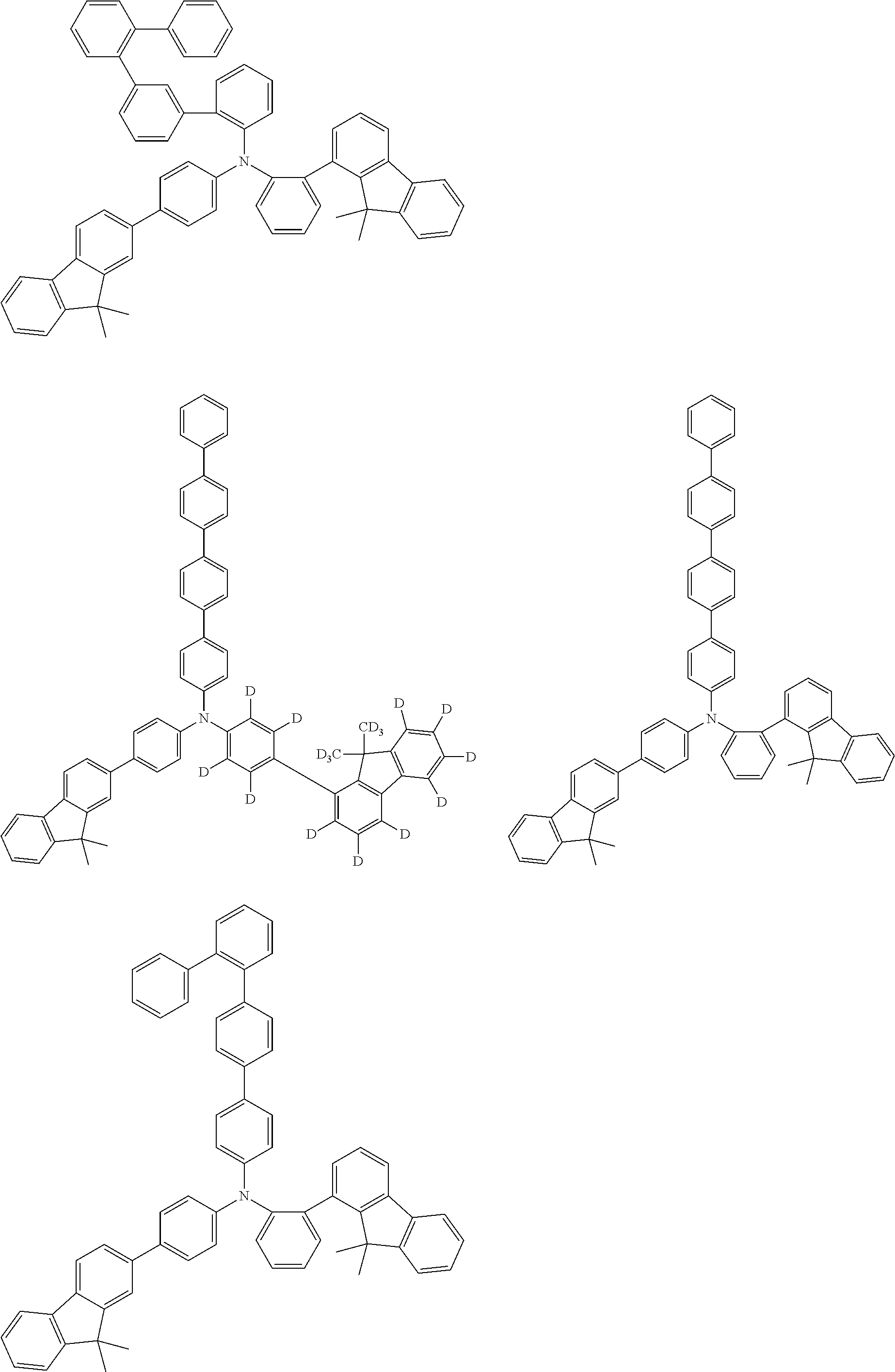

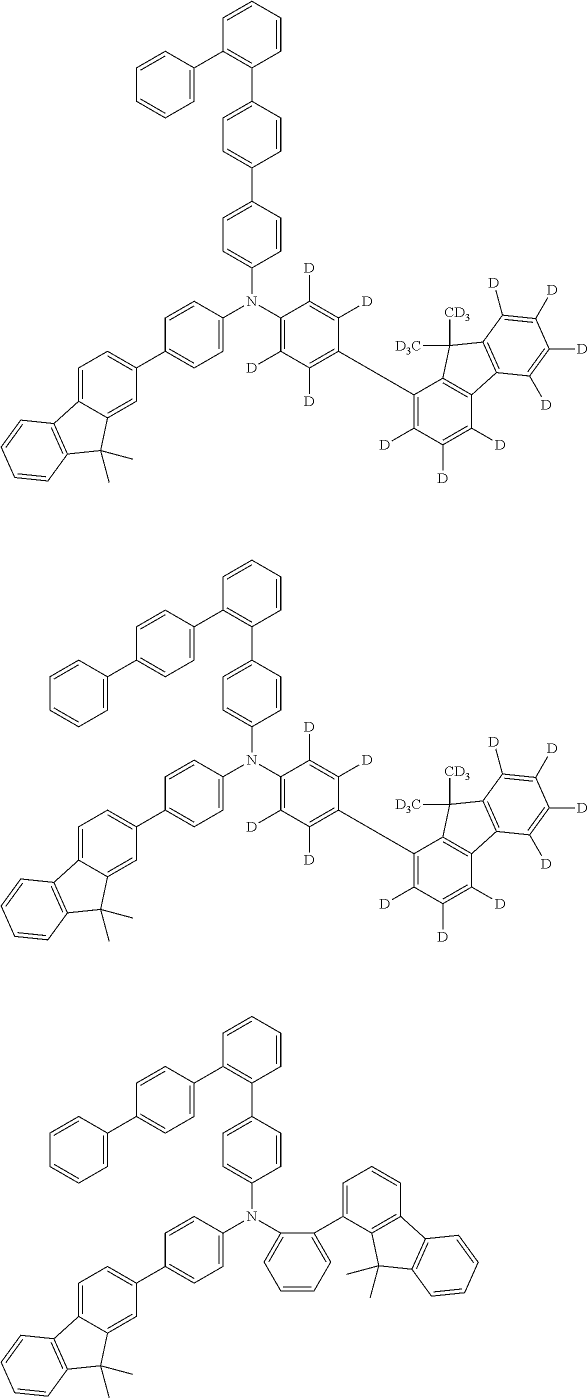

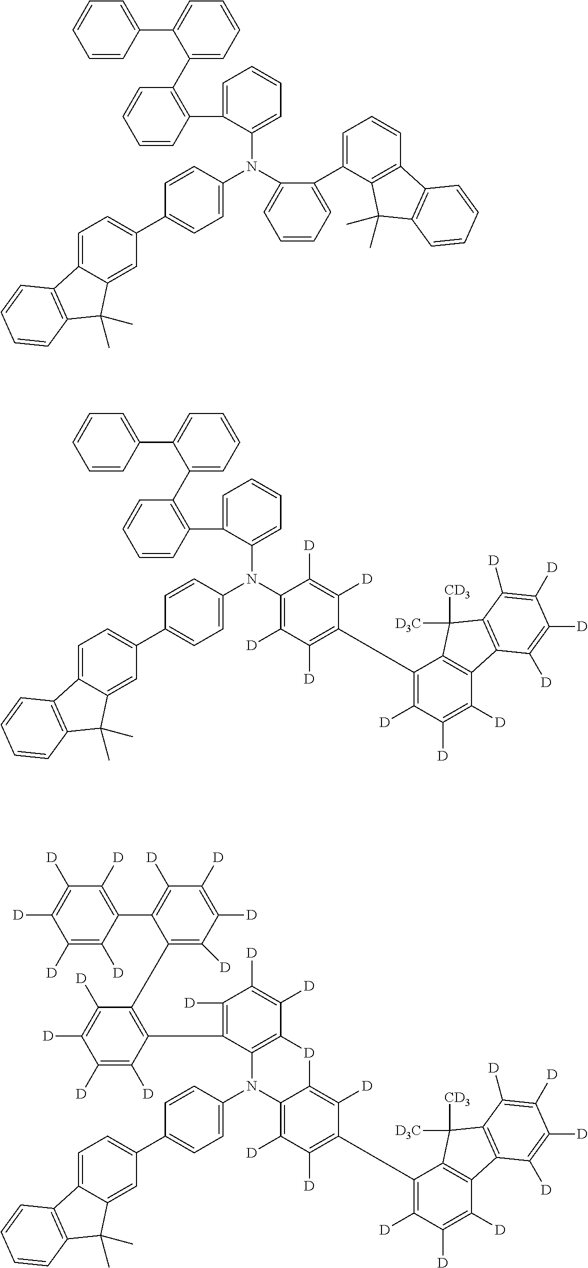

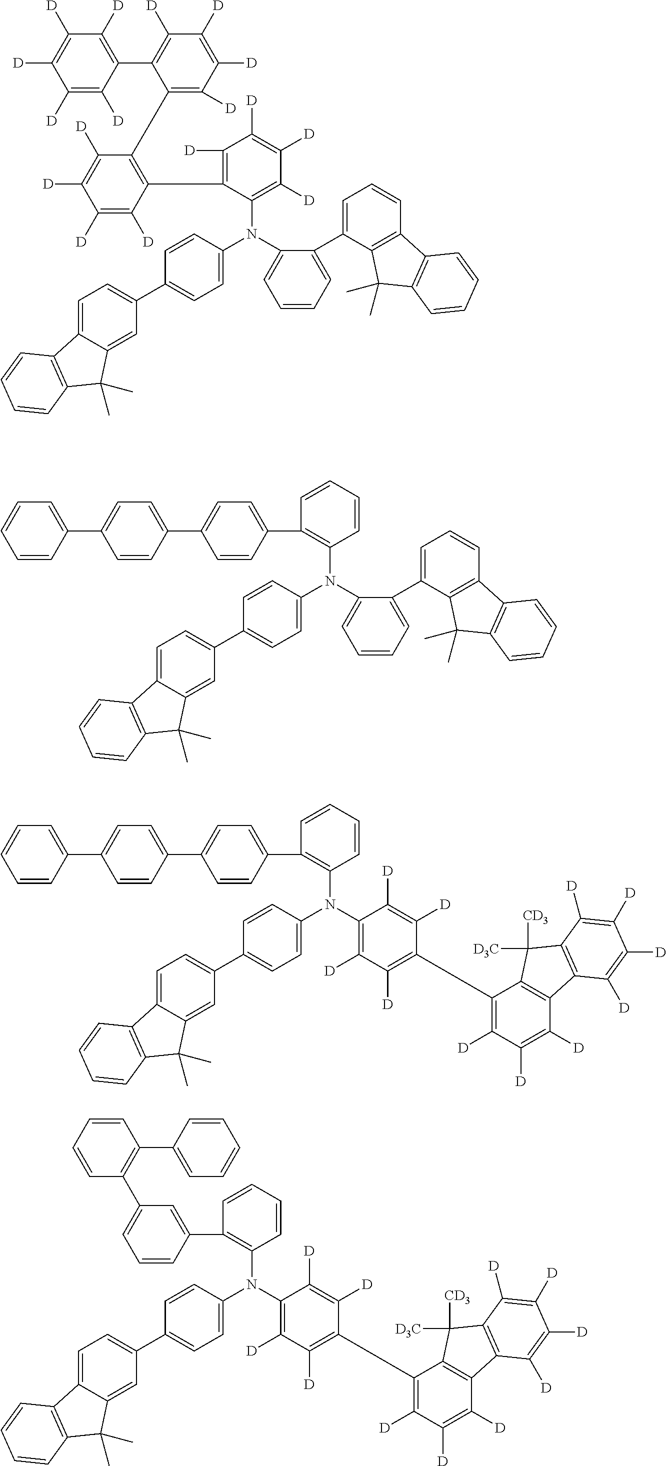

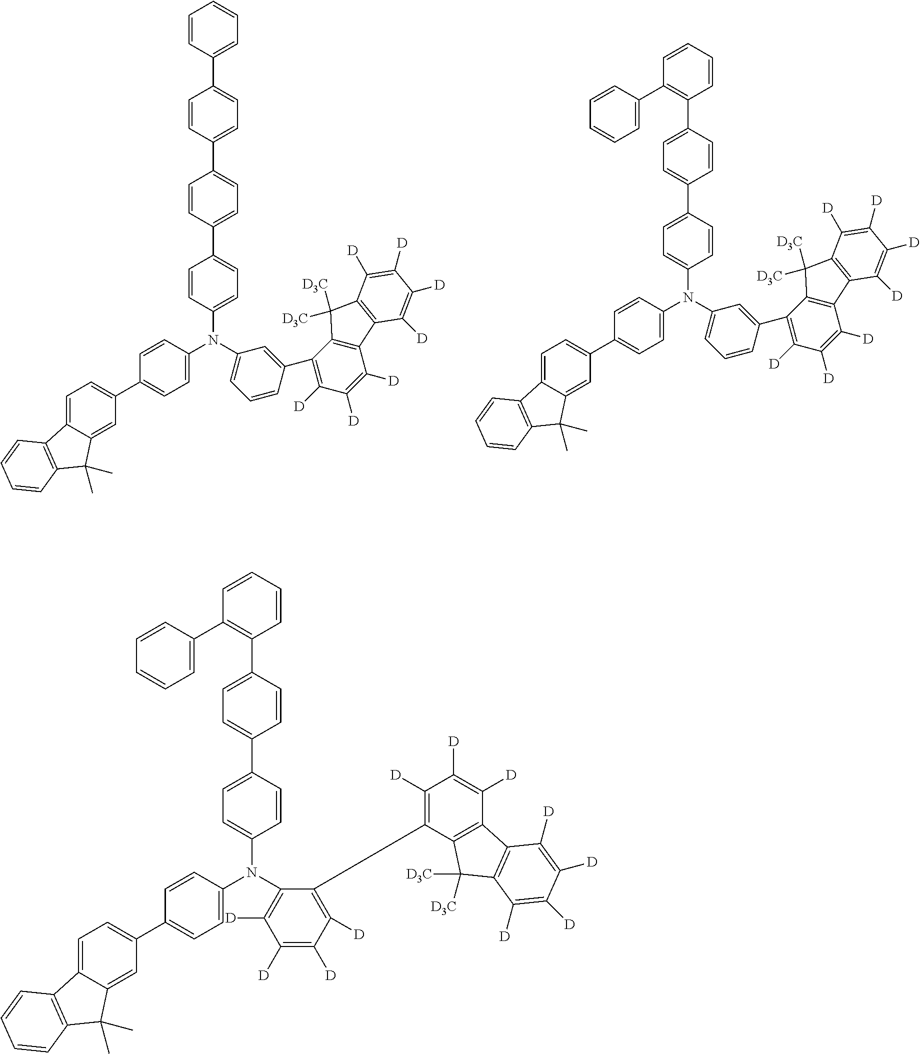

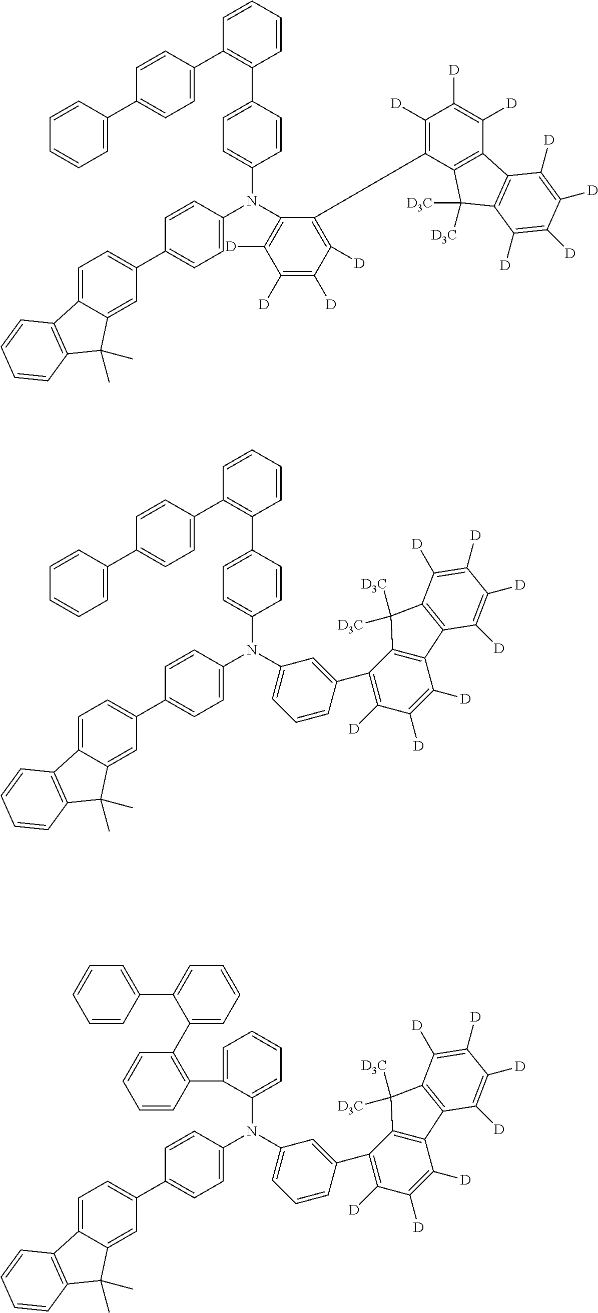

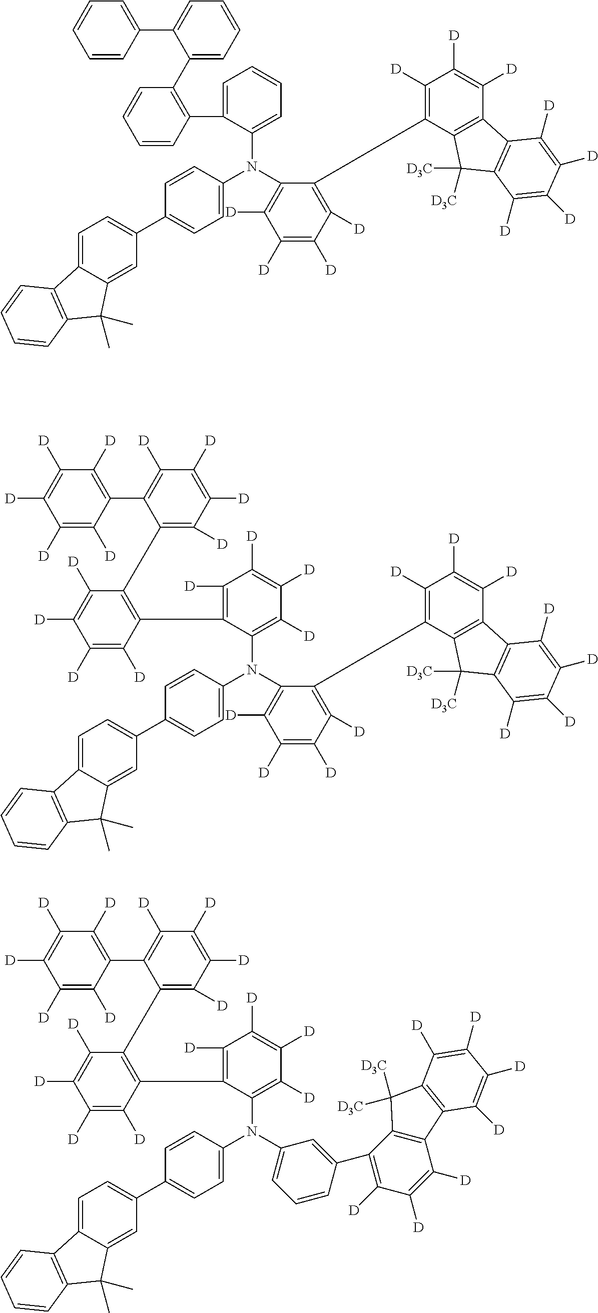

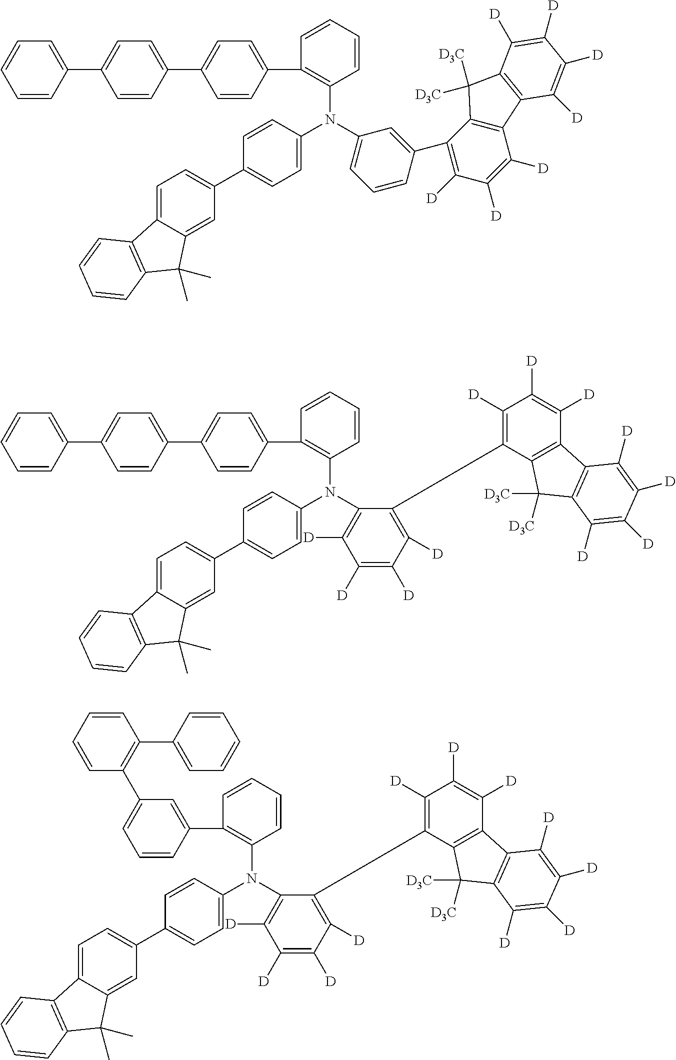

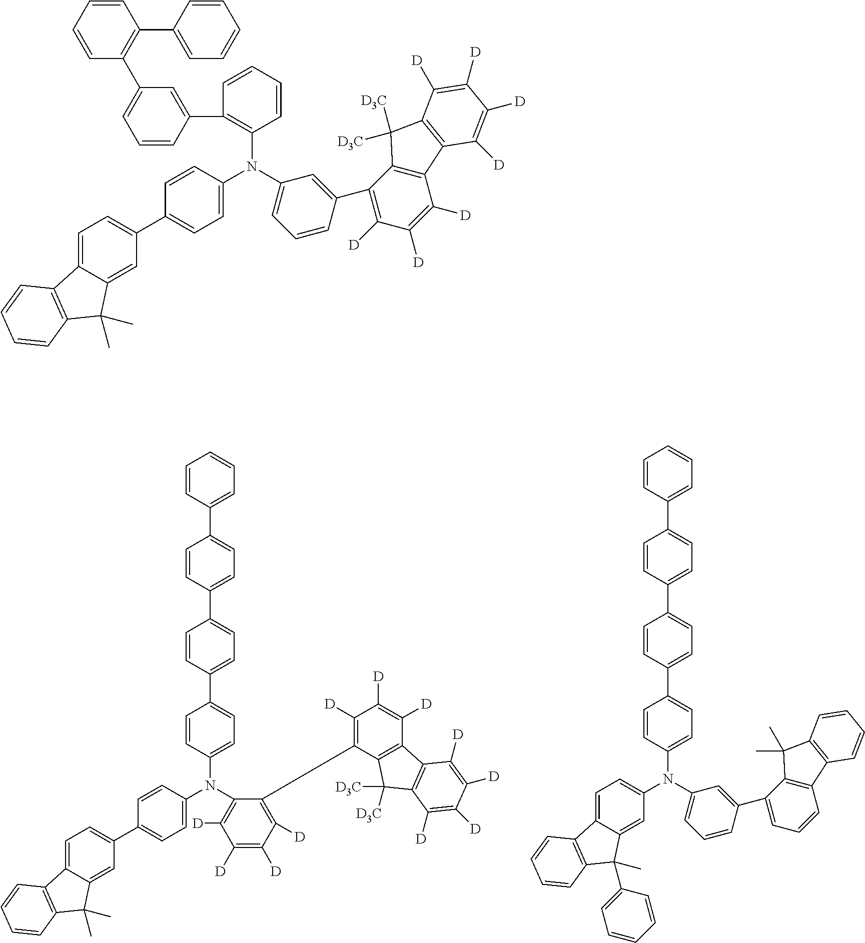

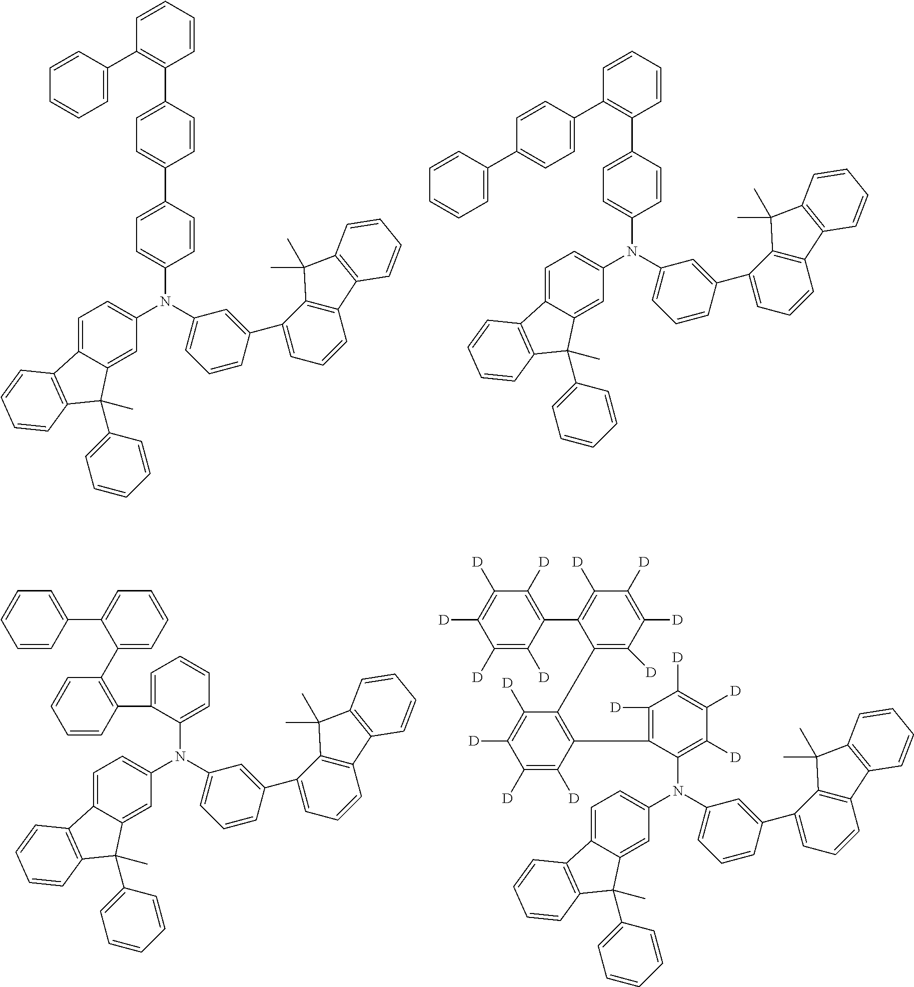

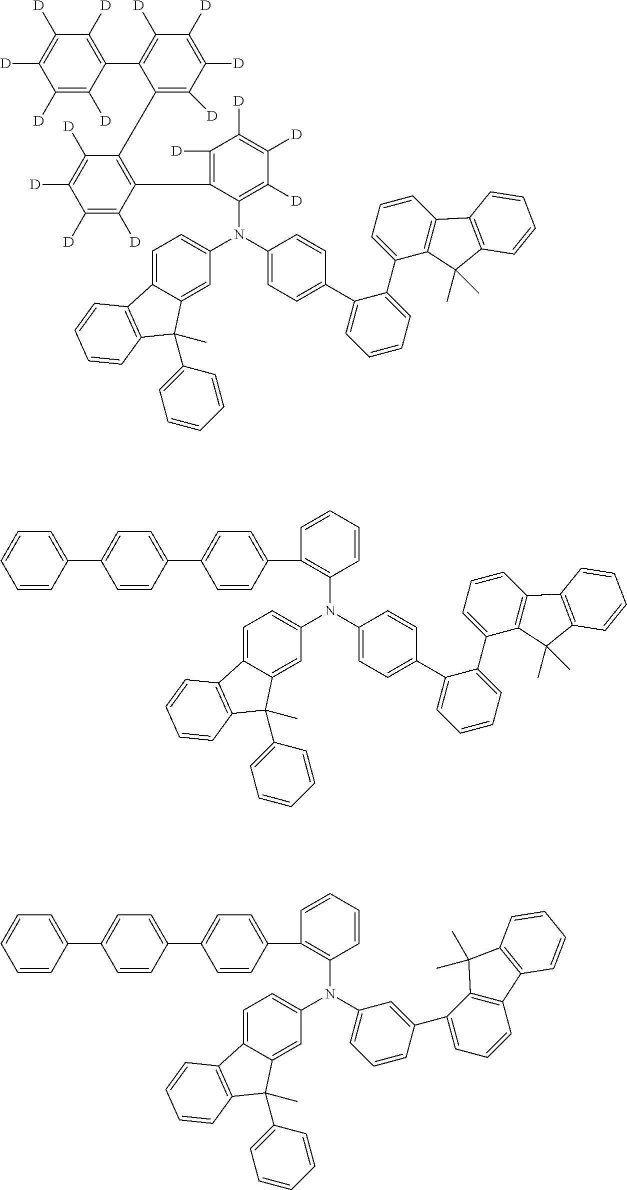 [SMILES: [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2ccc(-c3ccccc3-c3cccc4c3C(C)(C)c3ccccc3-4)cc2)c2ccc3c(c2)C(C)(c2ccccc2)c2ccccc2-3)c([2H])c1[2H] ; CC1(C)c2ccccc2-c2cccc(-c3ccccc3-c3ccc(N(c4ccc5c(c4)C(C)(c4ccccc4)c4ccccc4-5)c4ccccc4-c4ccc(-c5ccc(-c6ccccc6)cc5)cc4)cc3)c21 ; CC1(C)c2ccccc2-c2cccc(-c3cccc(N(c4ccc5c(c4)C(C)(c4ccccc4)c4ccccc4-5)c4ccccc4-c4ccc(-c5ccc(-c6ccccc6)cc5)cc4)c3)c21]

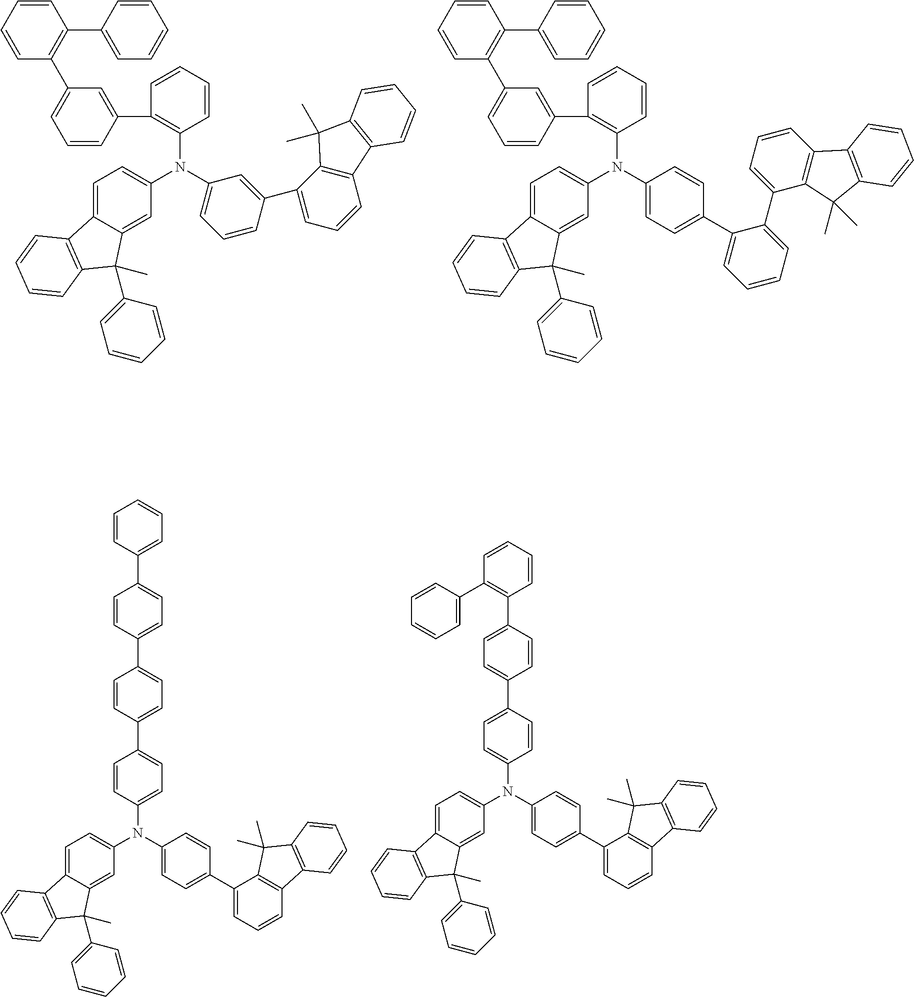

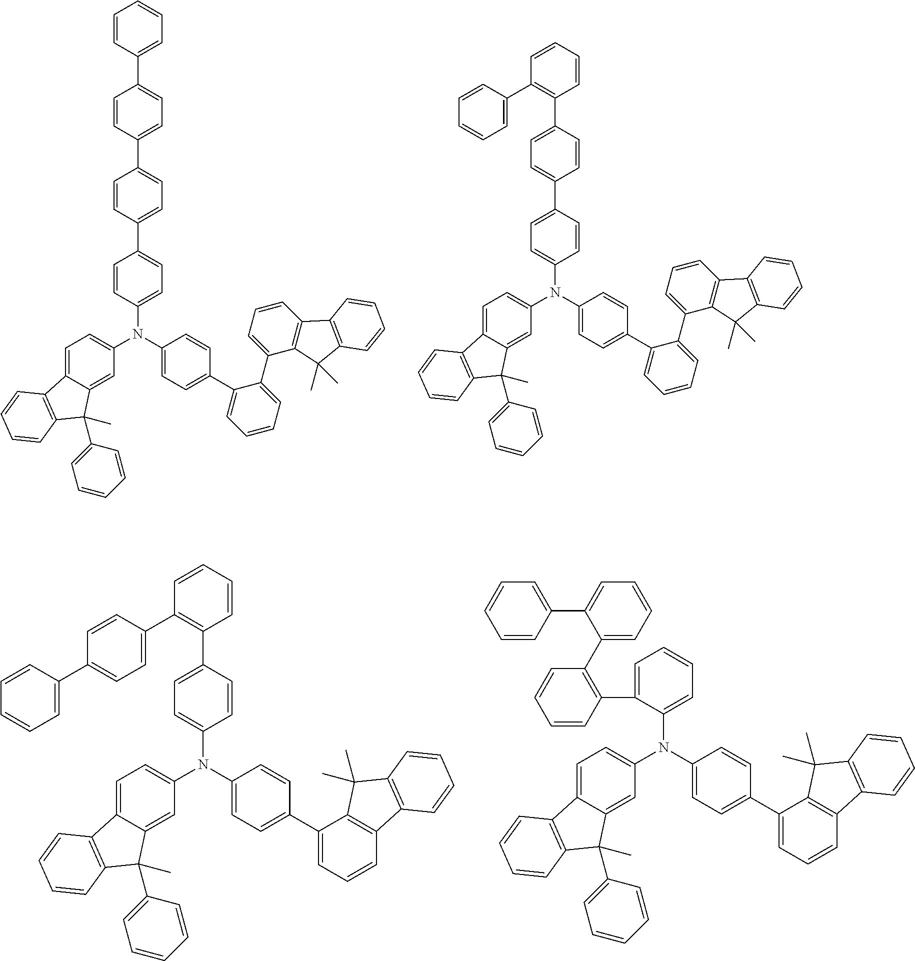 [SMILES: CC1(C)c2ccccc2-c2cccc(-c3ccccc3-c3ccc(N(c4ccc(-c5ccc(-c6ccc(-c7ccccc7)cc6)cc5)cc4)c4ccc5c(c4)C(C)(c4ccccc4)c4ccccc4-5)cc3)c21 ; CC1(C)c2ccccc2-c2cccc(-c3ccccc3-c3ccc(N(c4ccc(-c5ccc(-c6ccccc6-c6ccccc6)cc5)cc4)c4ccc5c(c4)C(C)(c4ccccc4)c4ccccc4-5)cc3)c21 ; CC1(C)c2ccccc2-c2cccc(-c3ccc(N(c4ccc(-c5ccccc5-c5ccc(-c6ccccc6)cc5)cc4)c4ccc5c(c4)C(C)(c4ccccc4)c4ccccc4-5)cc3)c21 ; CC1(C)c2ccccc2-c2cccc(-c3ccc(N(c4ccc5c(c4)C(C)(c4ccccc4)c4ccccc4-5)c4ccccc4-c4ccccc4-c4ccccc4-c4ccccc4)cc3)c21]

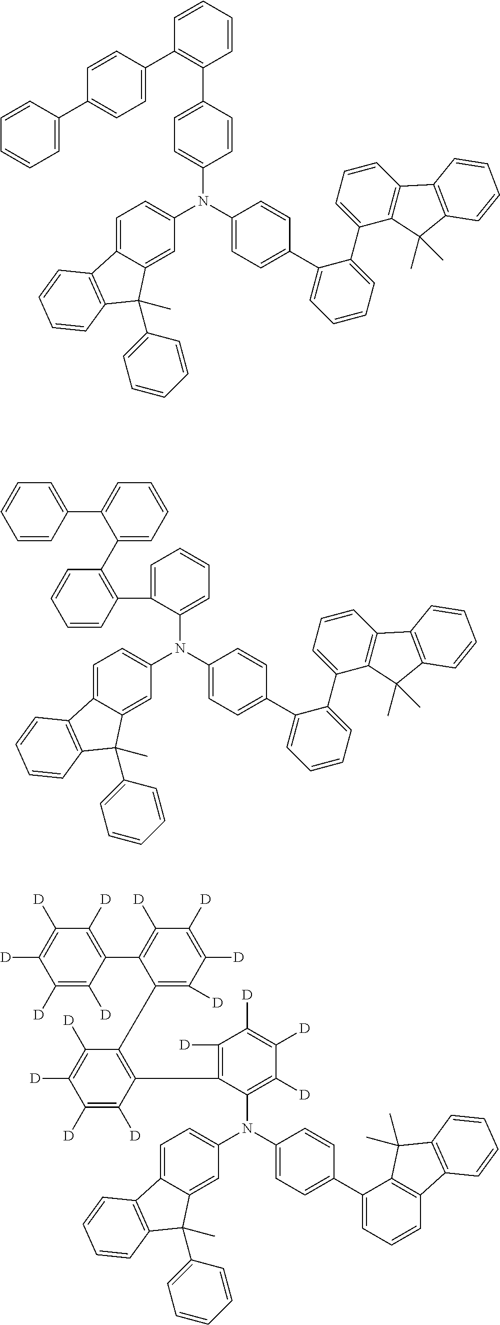 [SMILES: CC1(C)c2ccccc2-c2cccc(-c3ccccc3-c3ccc(N(c4ccc(-c5ccccc5-c5ccc(-c6ccccc6)cc5)cc4)c4ccc5c(c4)C(C)(c4ccccc4)c4ccccc4-5)cc3)c21 ; CC1(C)c2ccccc2-c2cccc(-c3ccccc3-c3ccc(N(c4ccc5c(c4)C(C)(c4ccccc4)c4ccccc4-5)c4ccccc4-c4ccccc4-c4ccccc4-c4ccccc4)cc3)c21 ; [2H]c1c([2H])c([2H])c(-c2c([2H])c([2H])c([2H])c([2H])c2-c2c([2H])c([2H])c([2H])c([2H])c2-c2c([2H])c([2H])c([2H])c([2H])c2N(c2ccc(-c3cccc4c3C(C)(C)c3ccccc3-4)cc2)c2ccc3c(c2)C(C)(c2ccccc2)c2ccccc2-3)c([2H])c1[2H]]

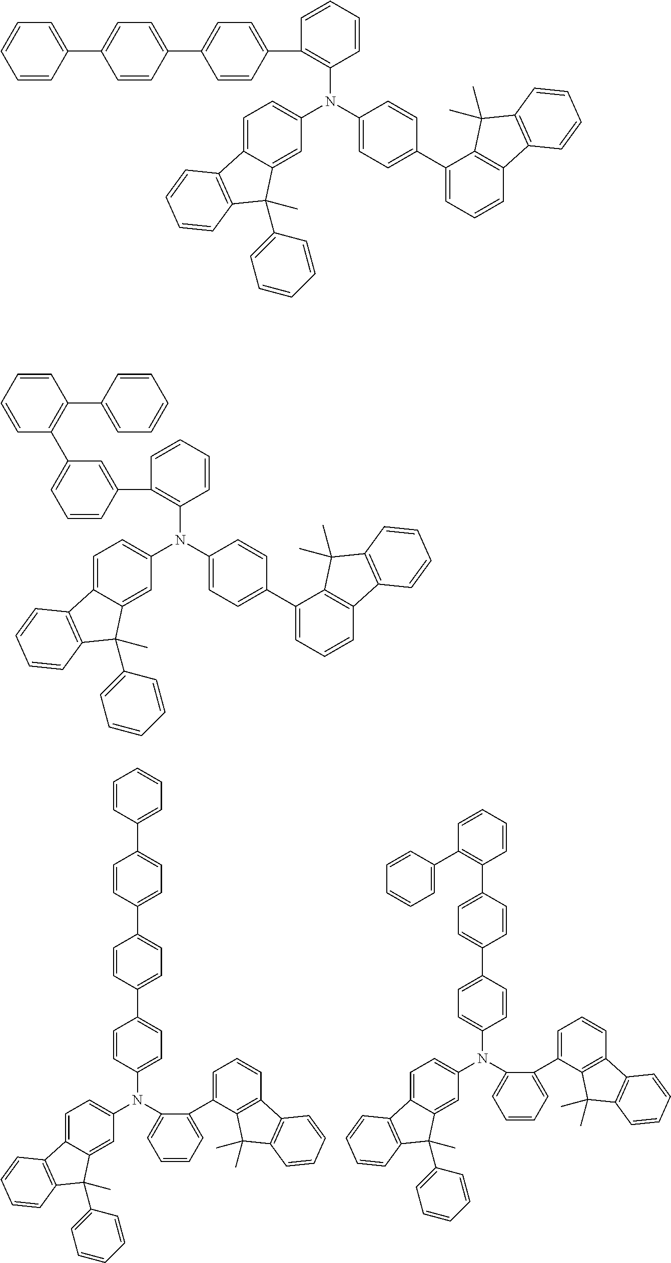

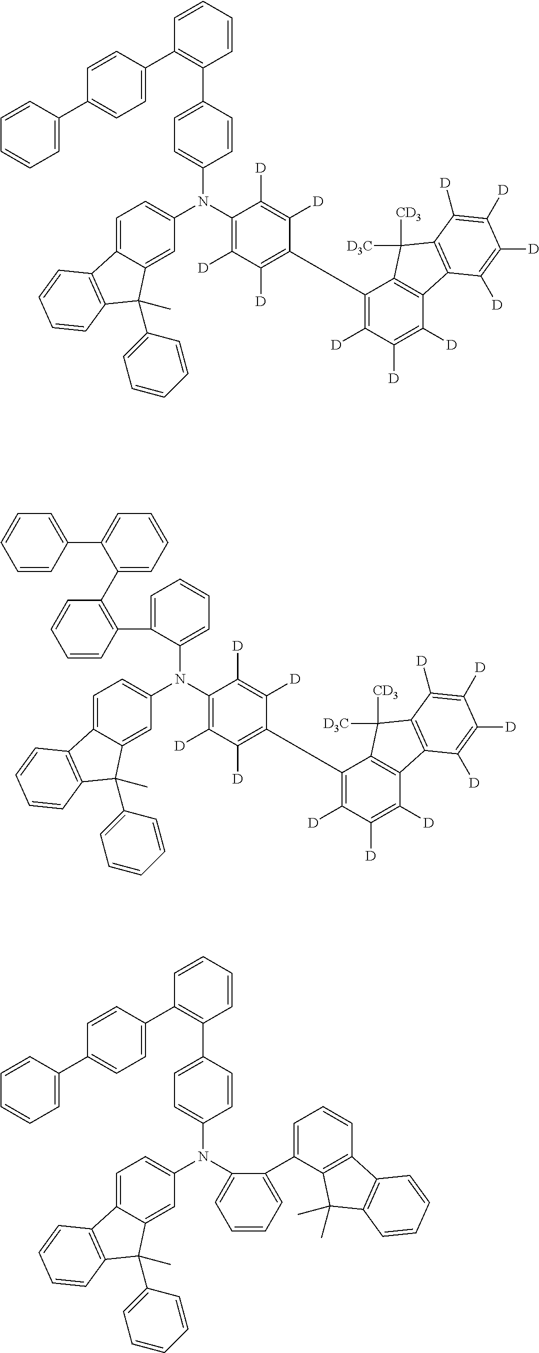

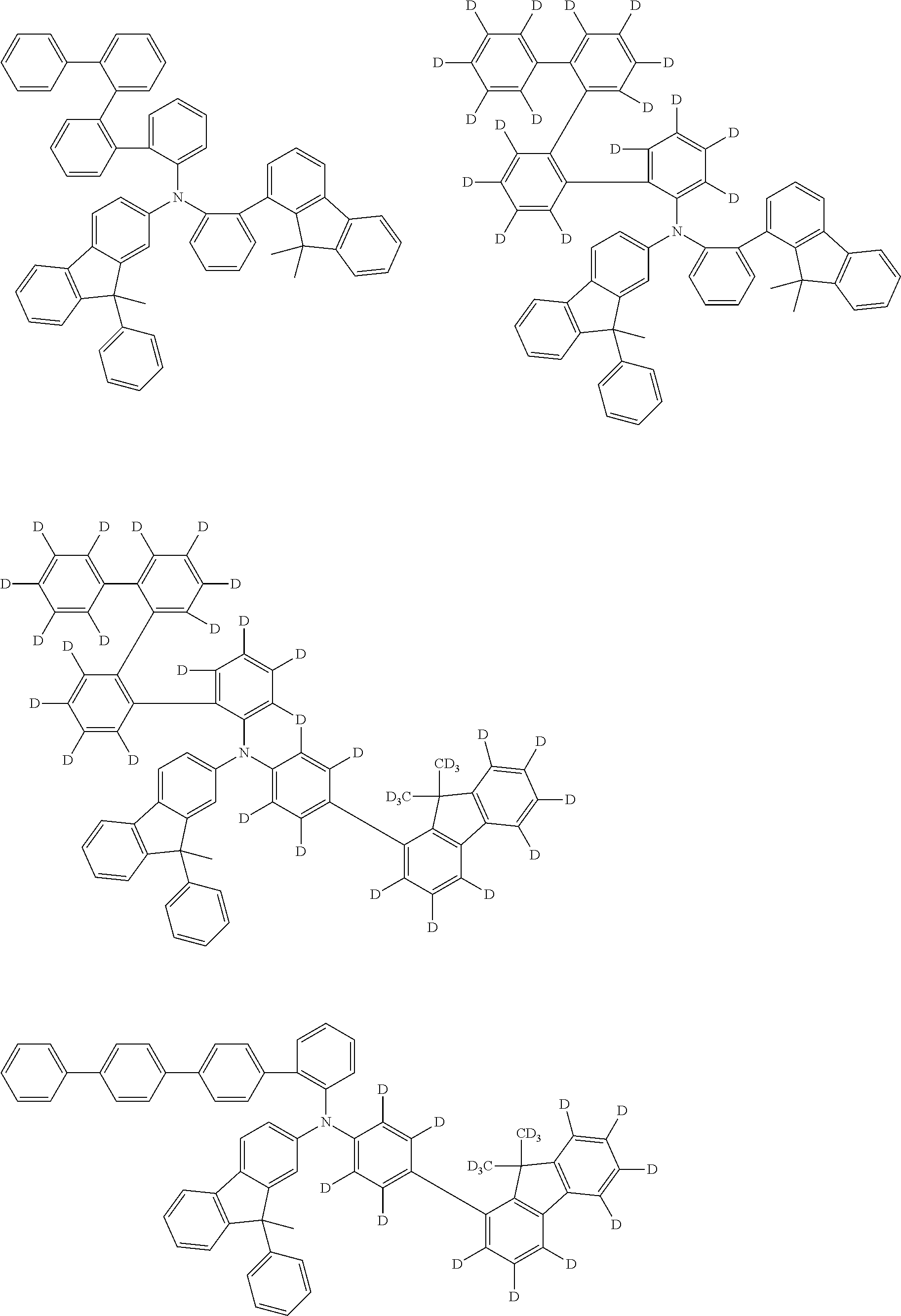

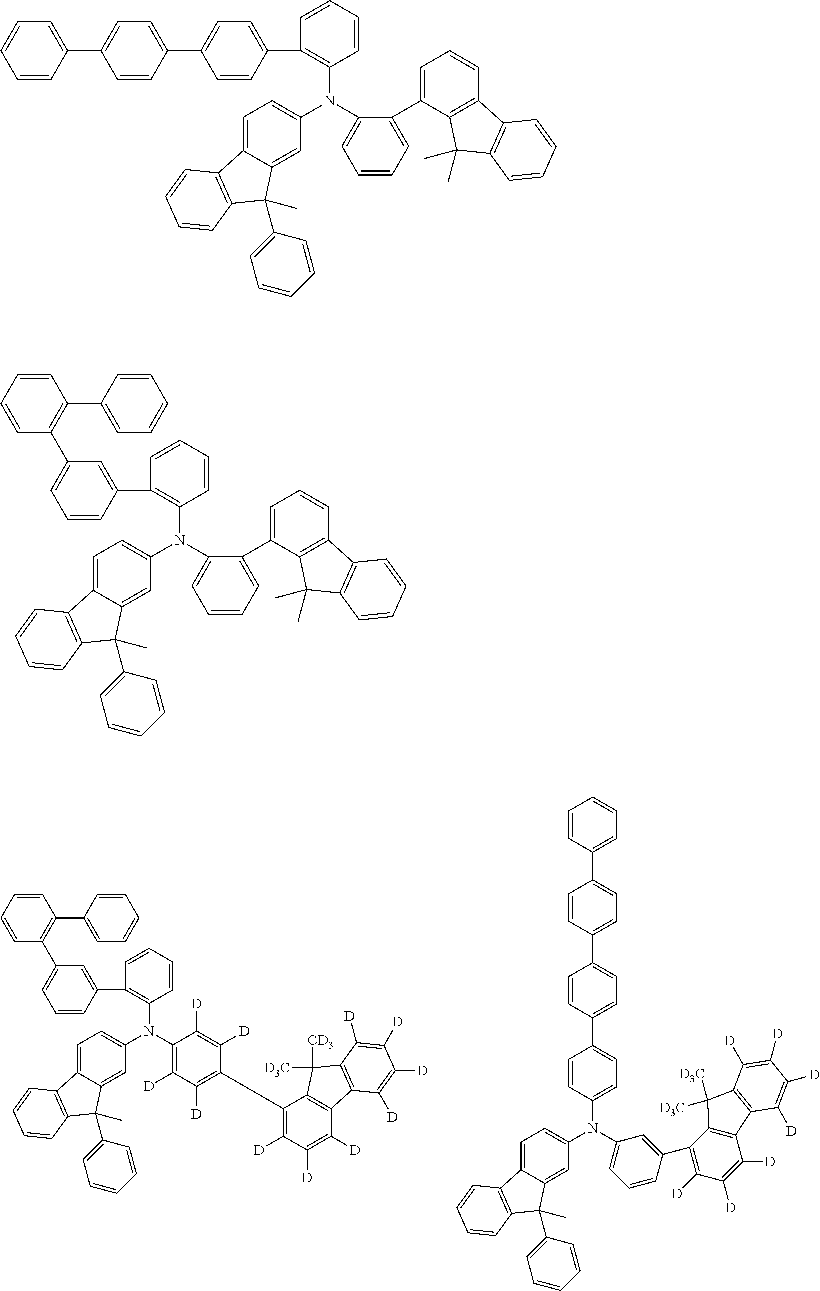

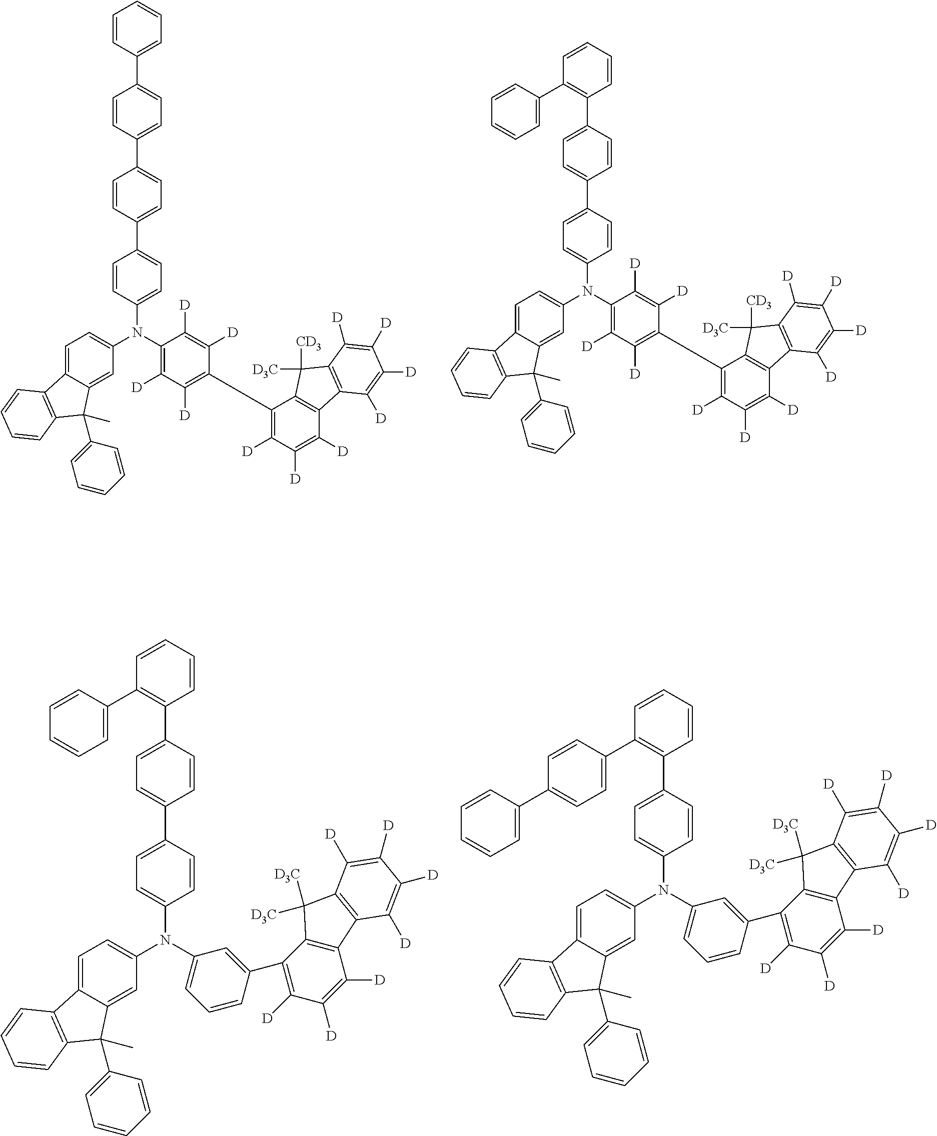

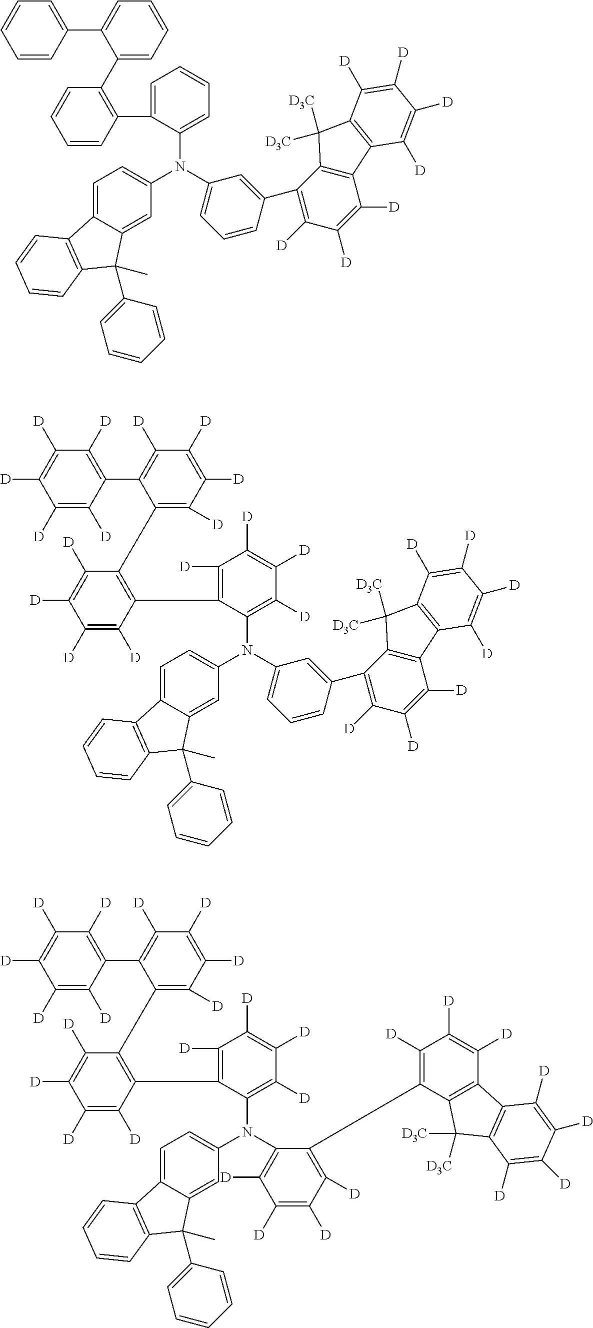

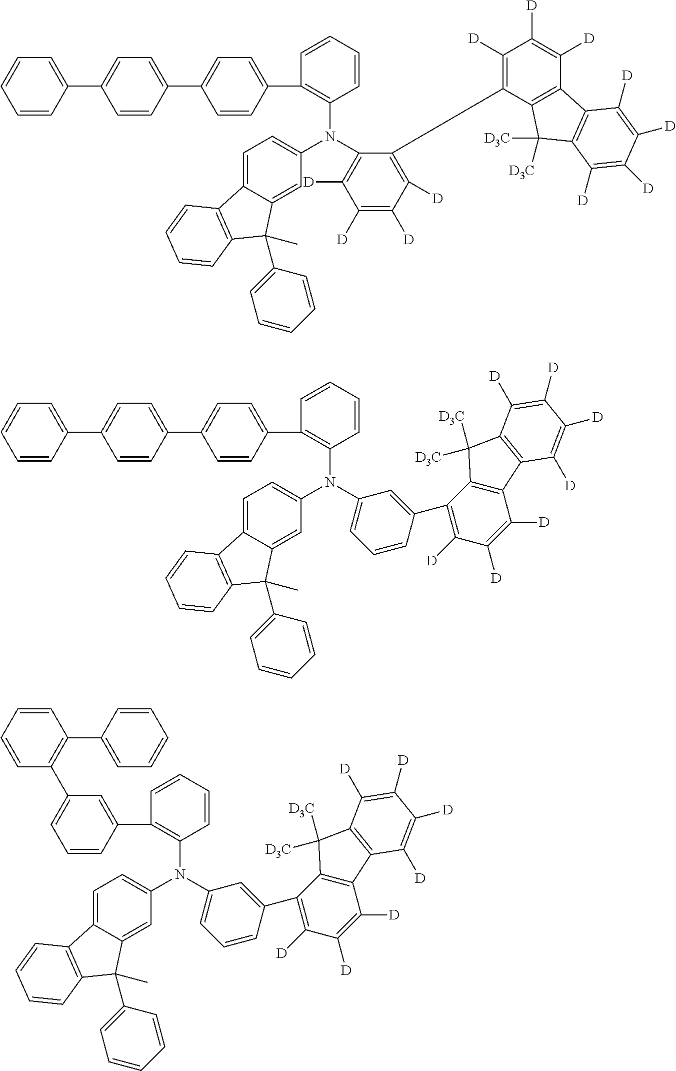

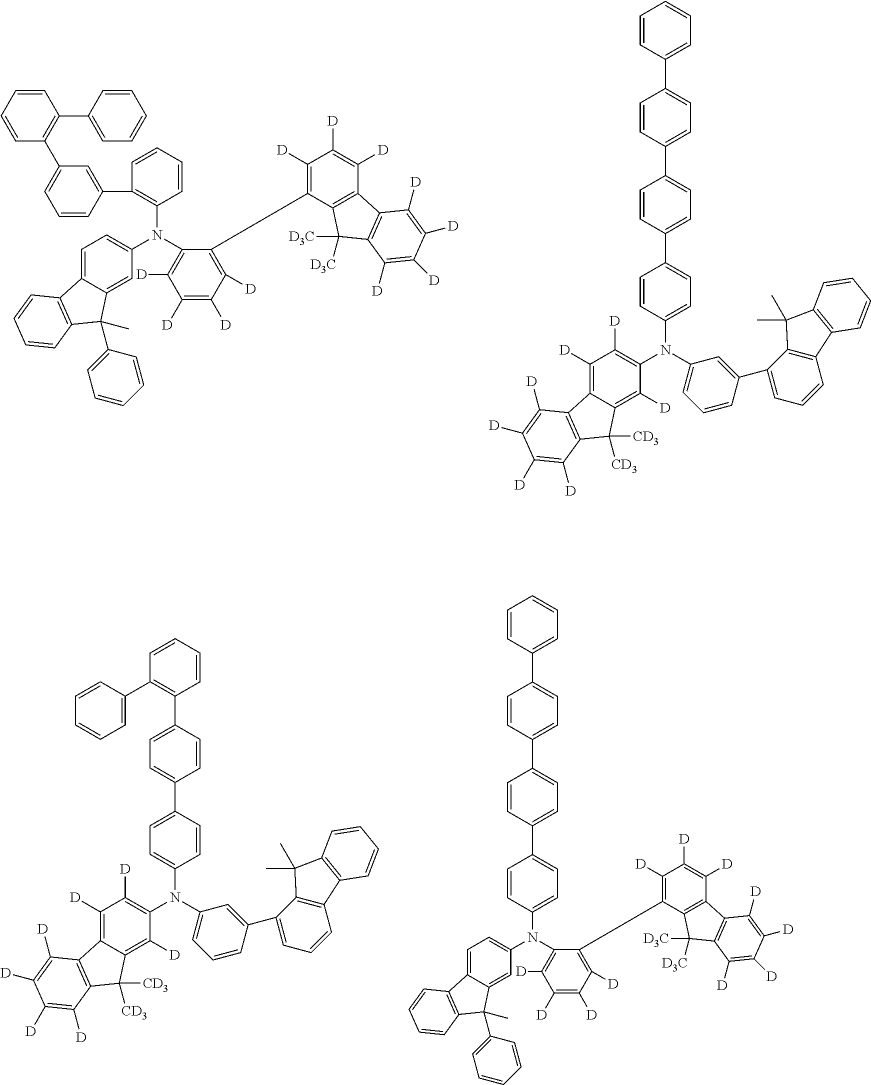

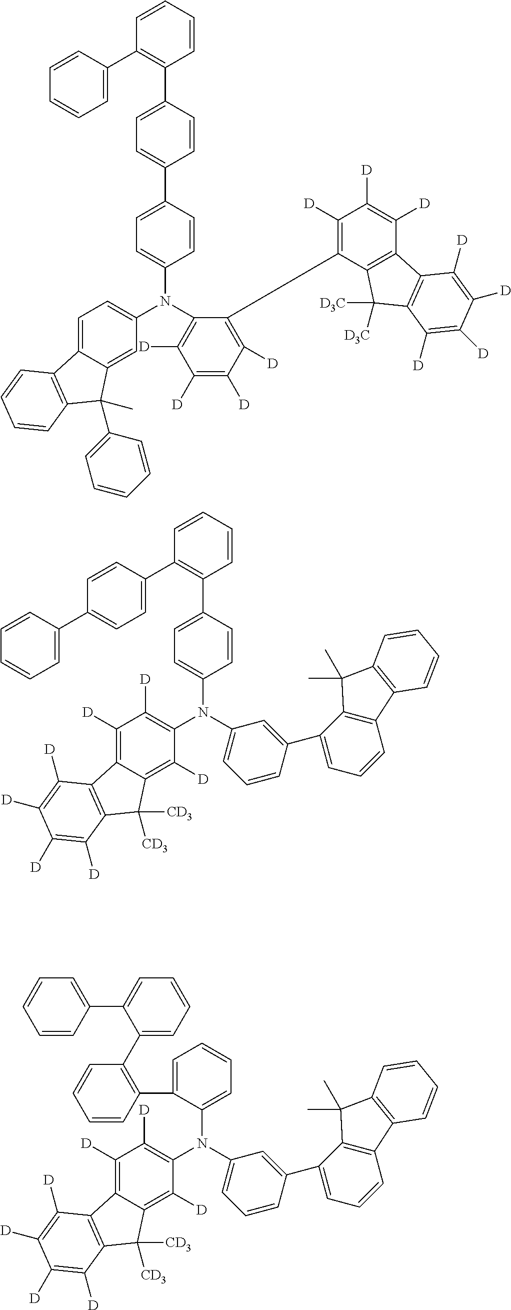

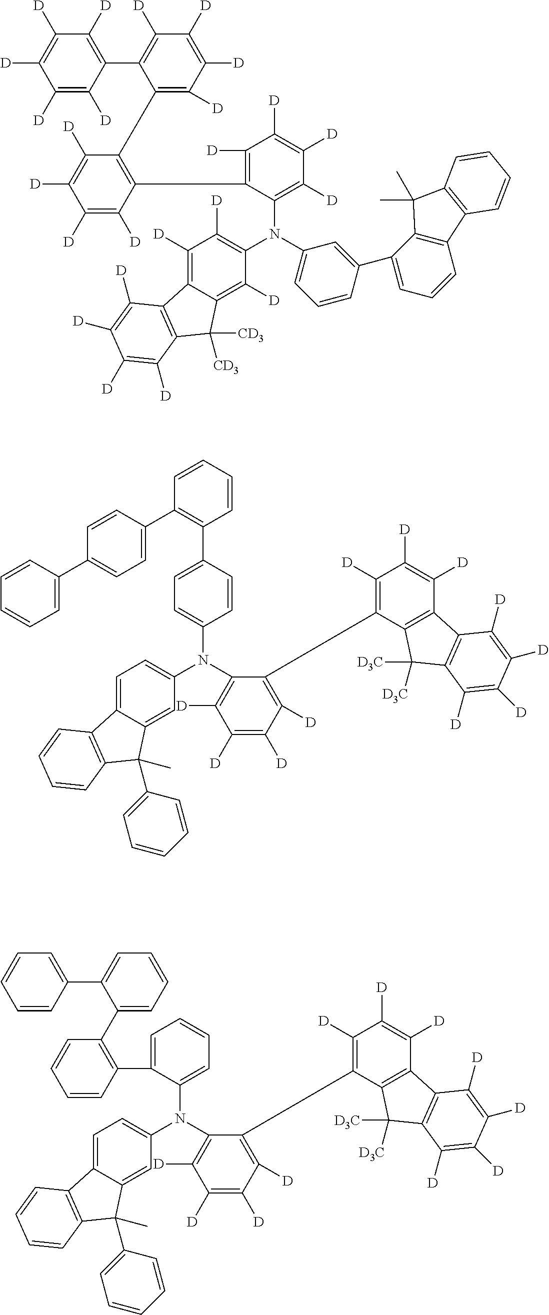

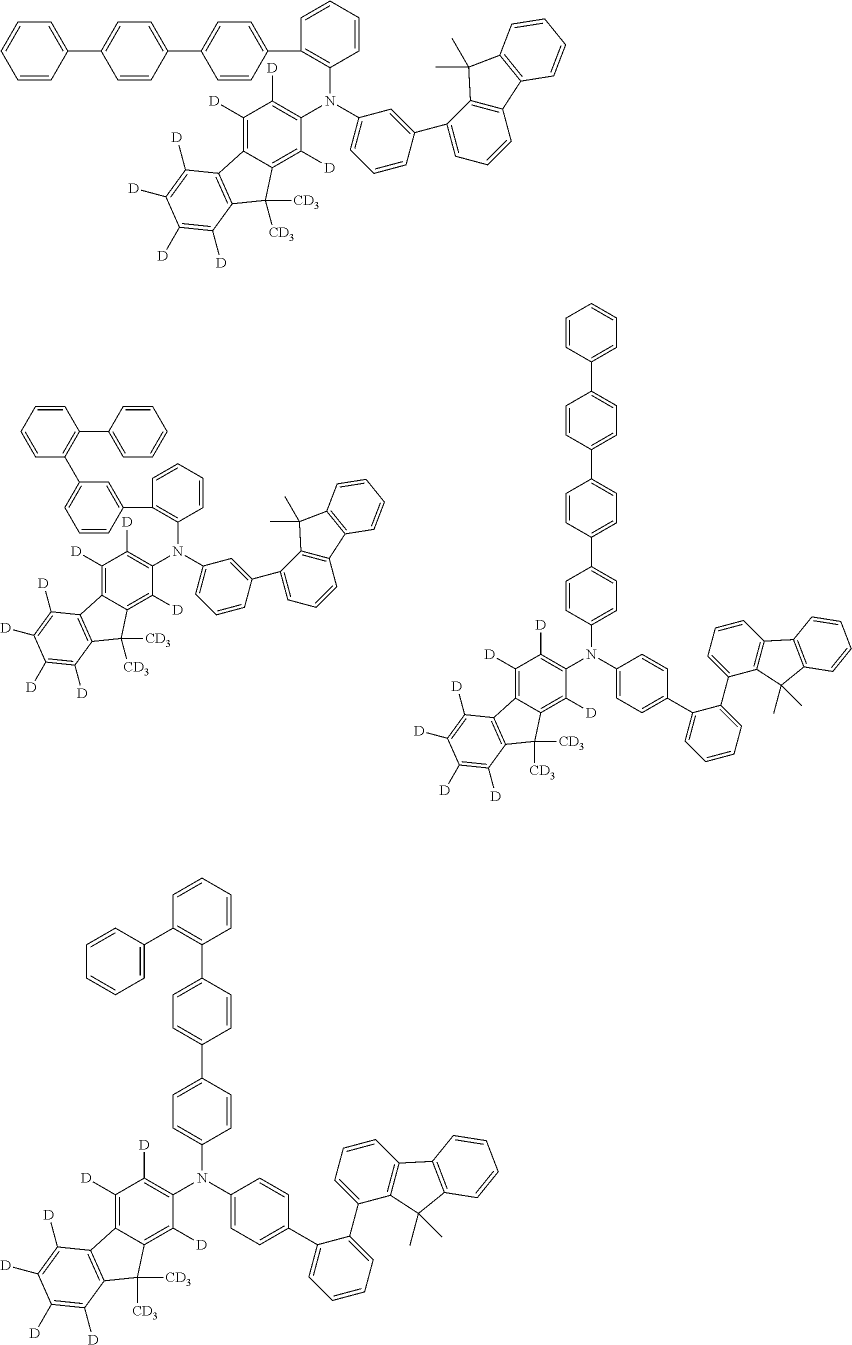

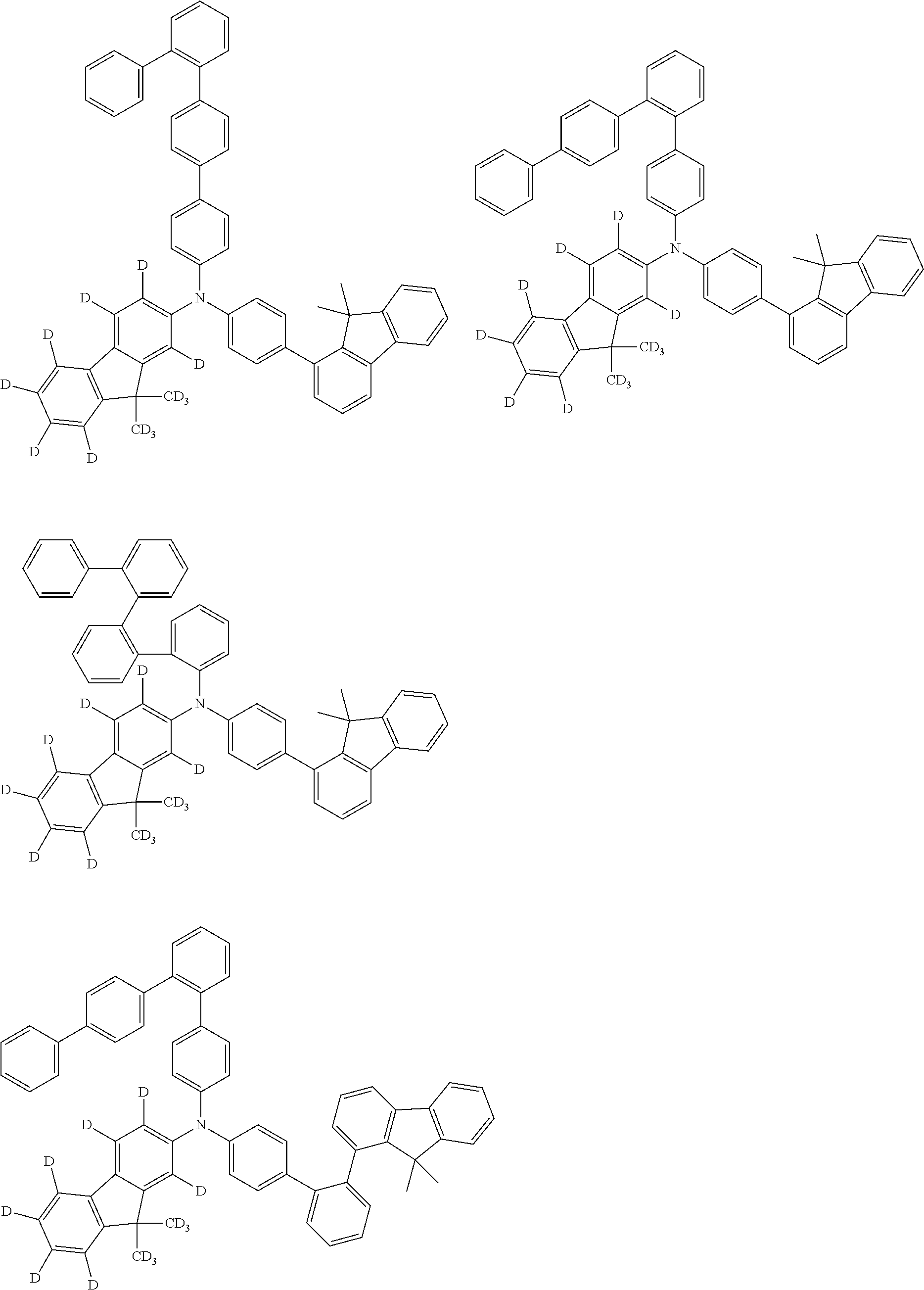

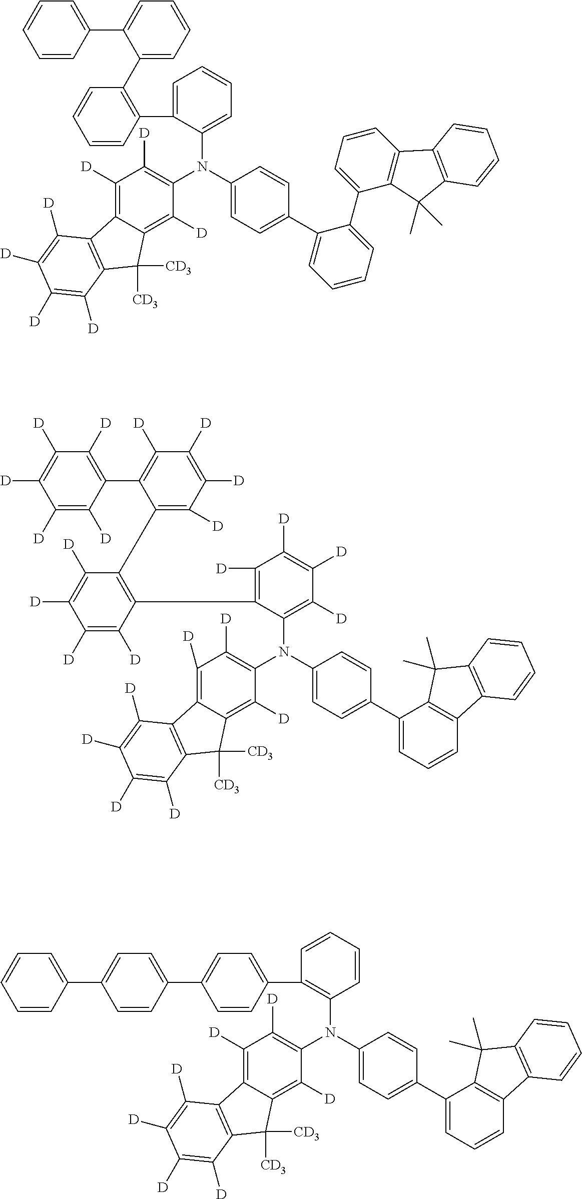

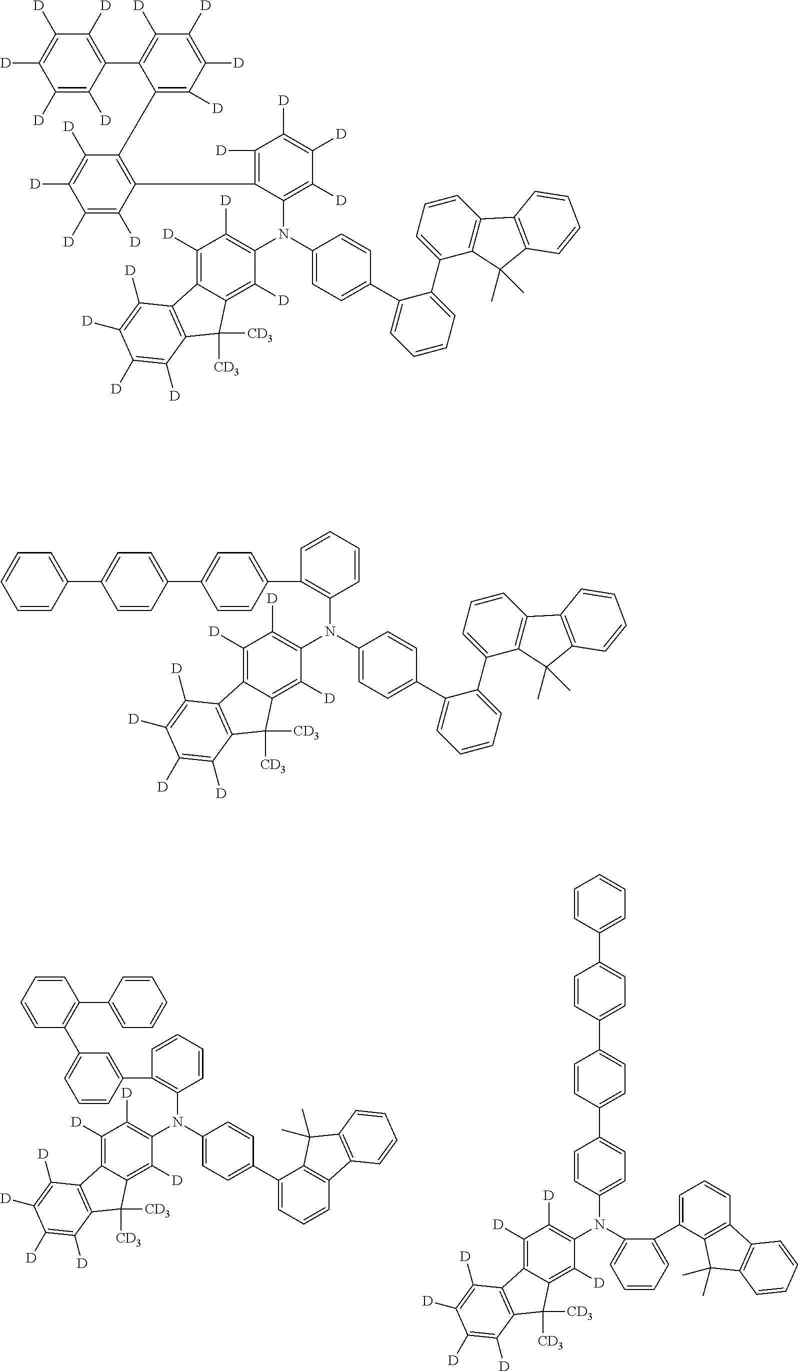

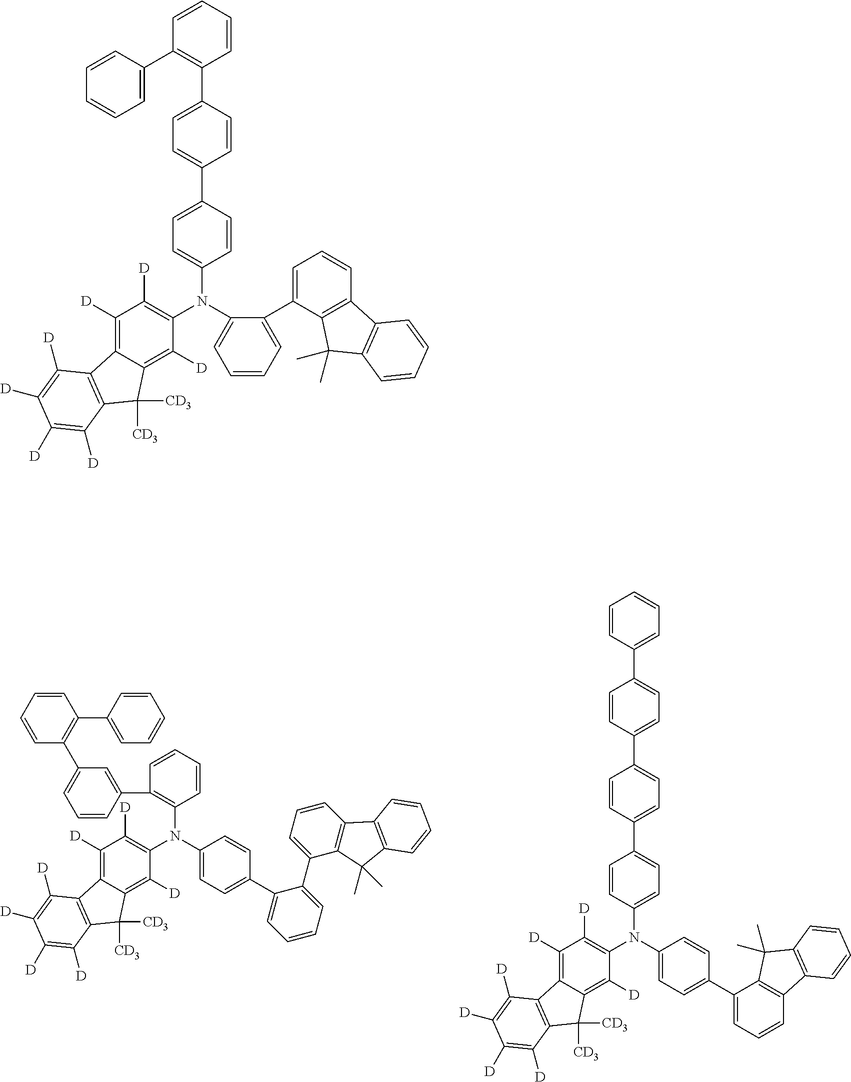

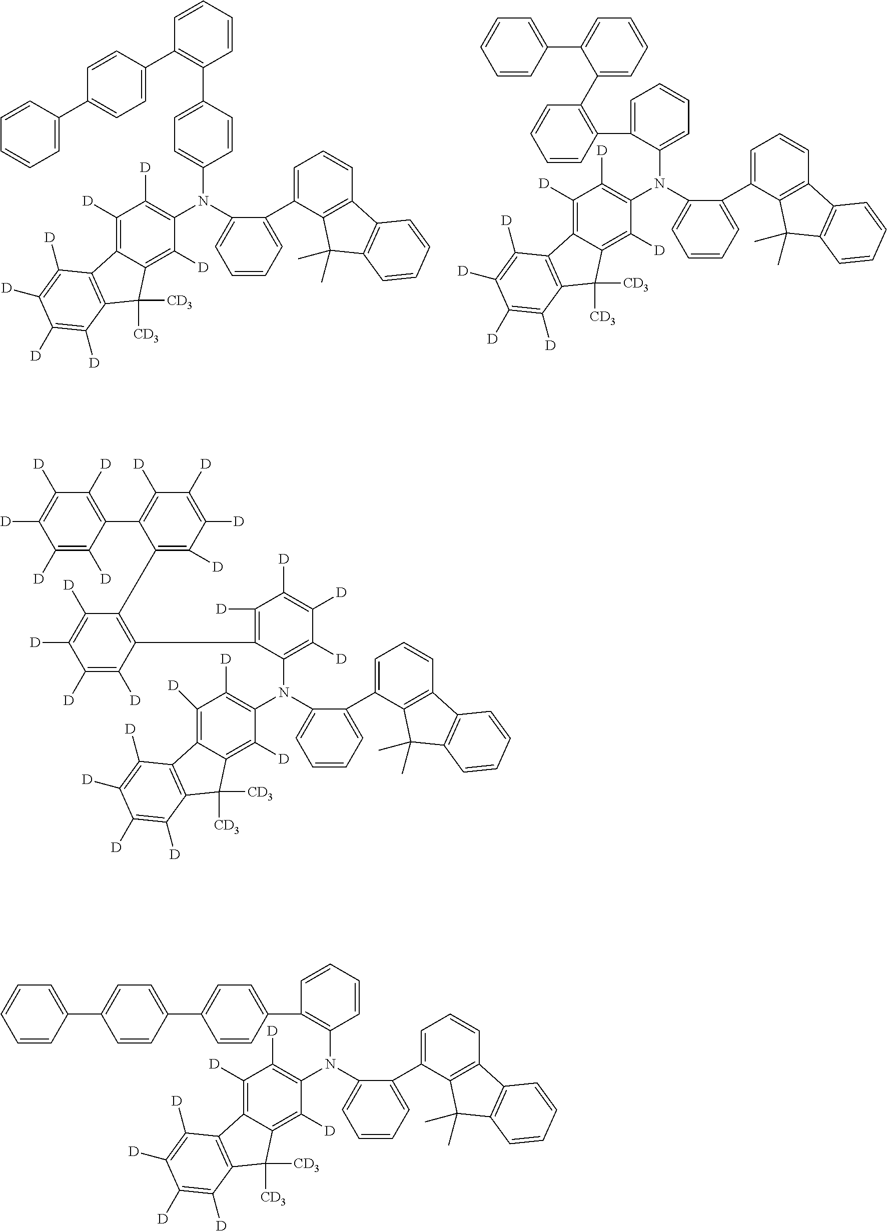

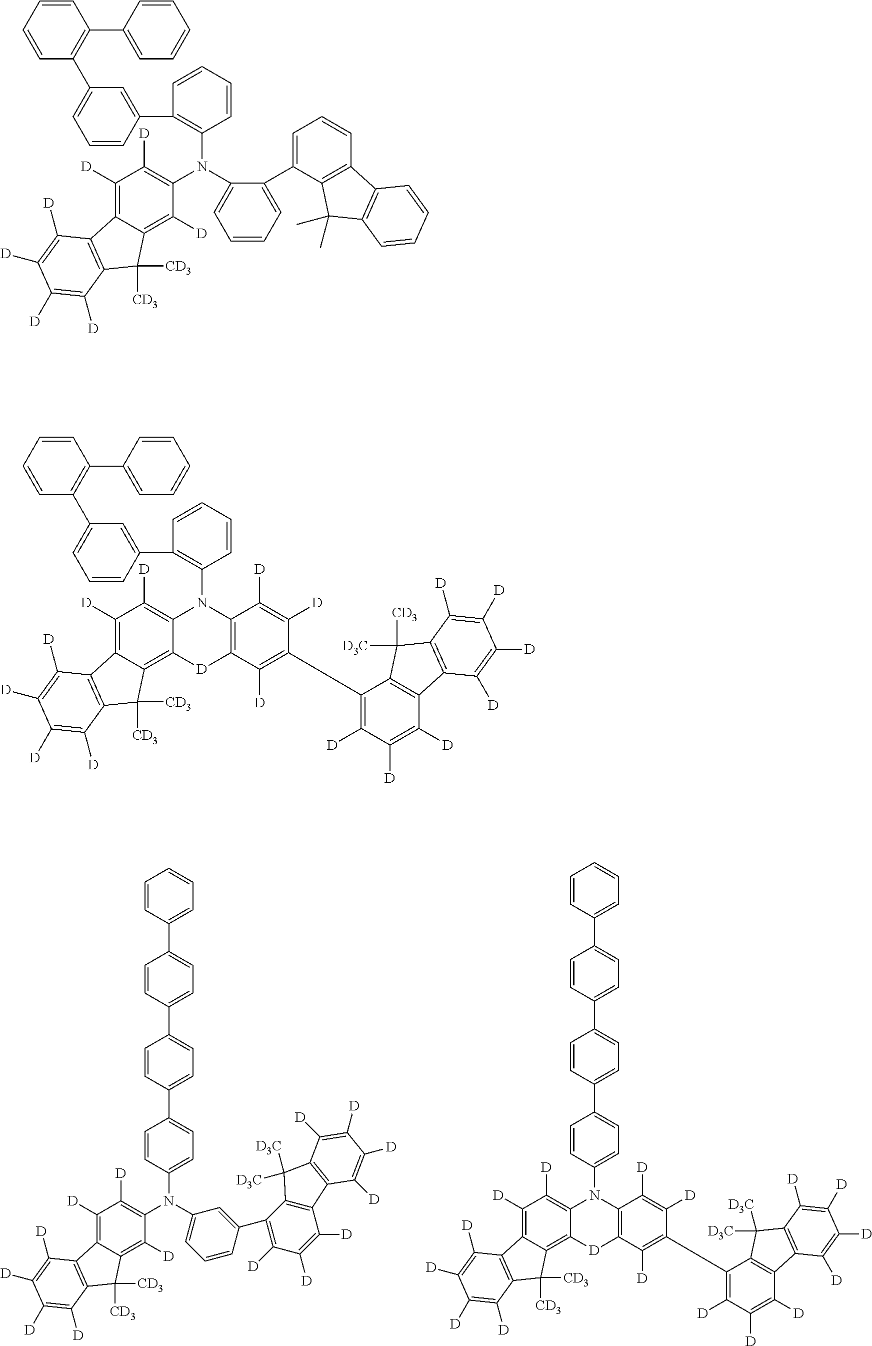

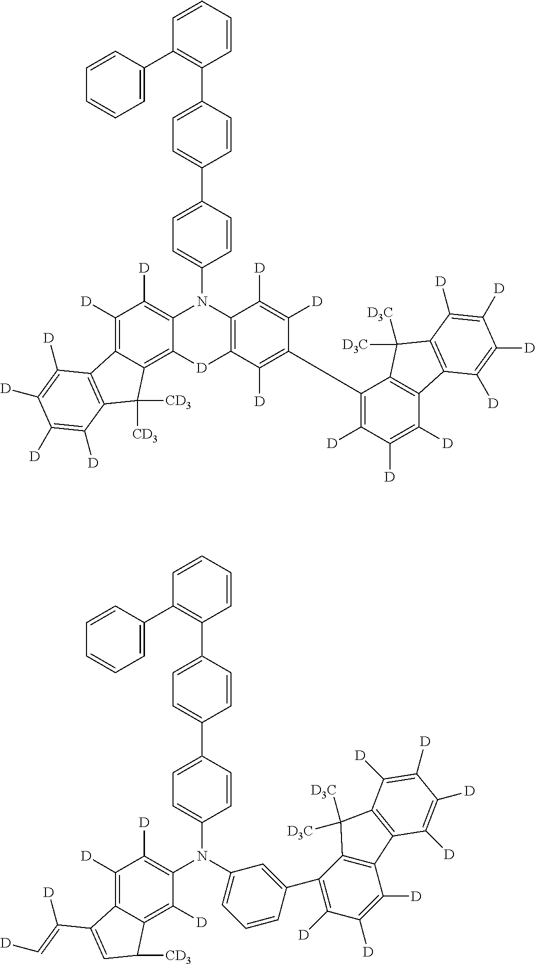

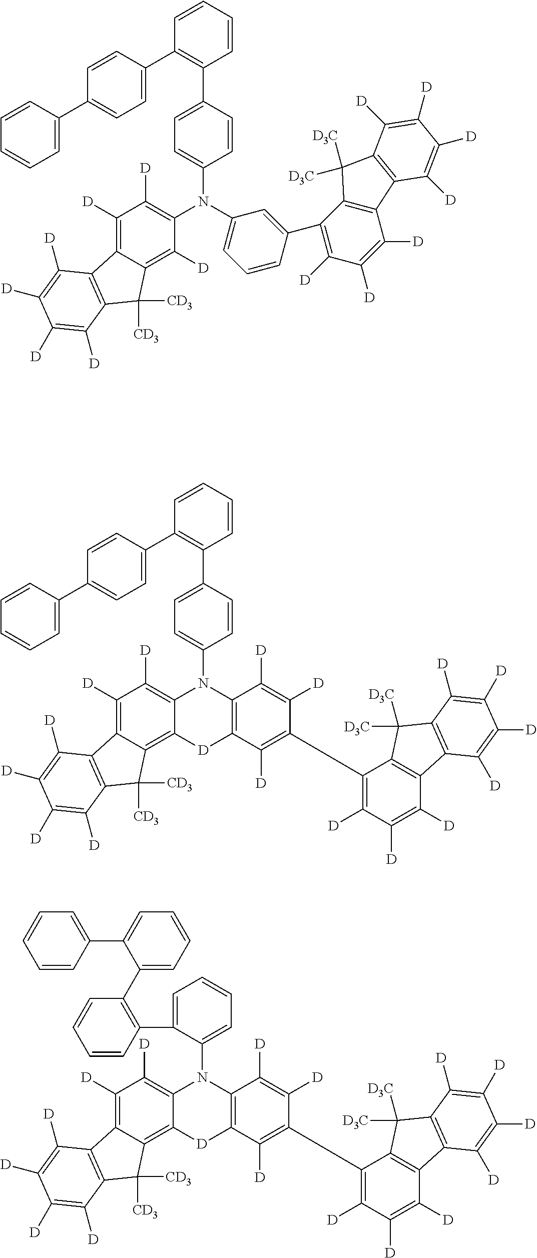

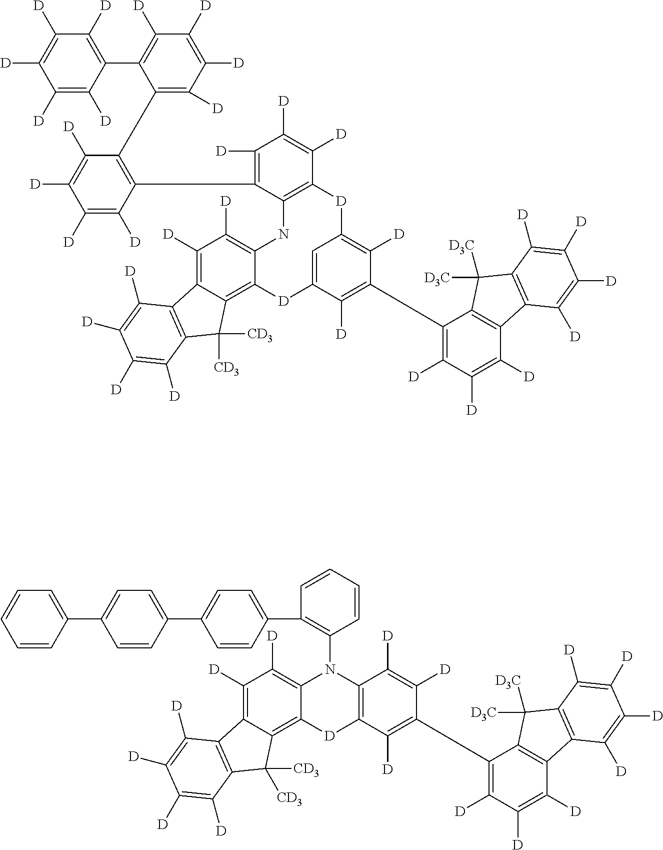

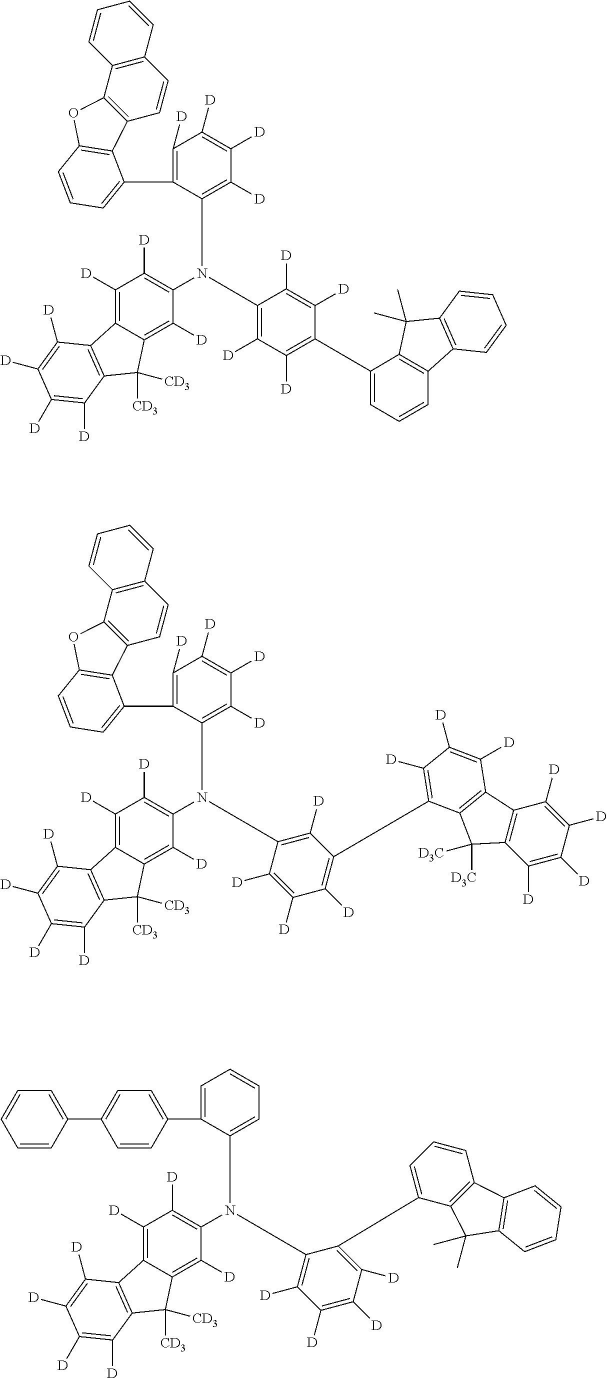

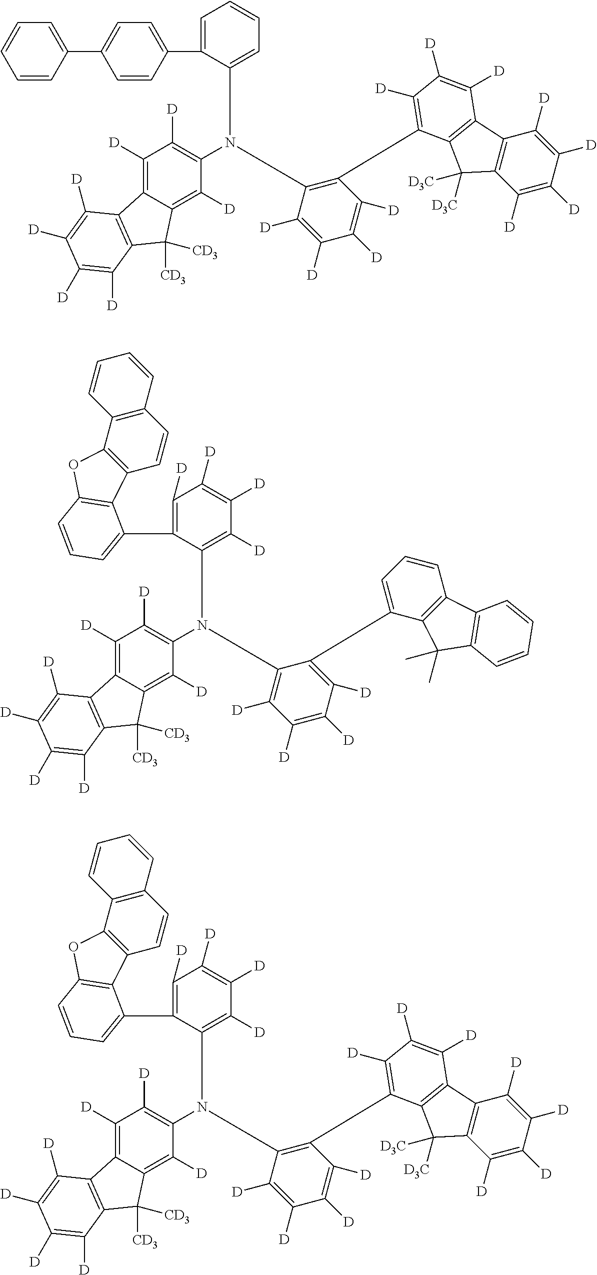

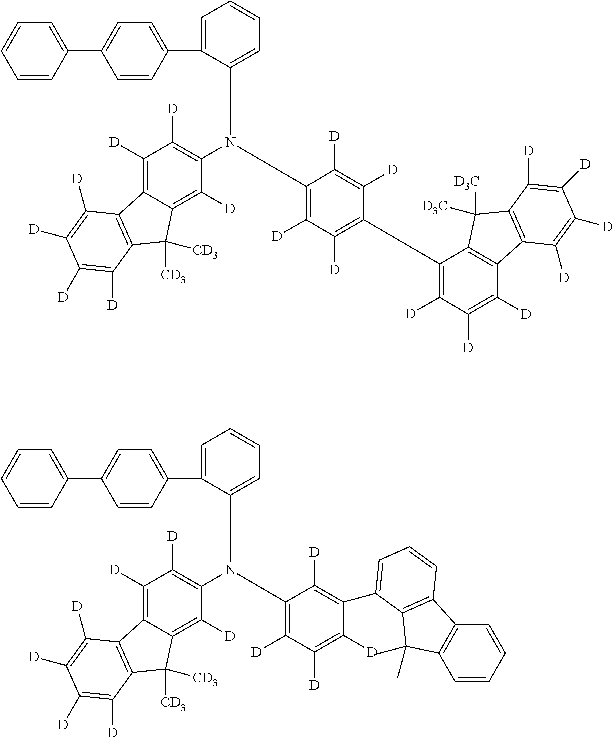 [SMILES: [2H]c1c([2H])c([2H])c2c(c1[2H])-c1c([2H])c([2H])c(N(c3ccccc3-c3ccc(-c4ccccc4)cc3)c3c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c([2H])c3[2H])c([2H])c1C2(C([2H])([2H])[2H])C([2H])([2H])[2H] ; [2H]c1c([2H])c([2H])c2c(c1[2H])-c1c([2H])c([2H])c(N(c3ccccc3-c3ccc(-c4ccccc4)cc3)c3c([2H])c([2H])c([2H])c(-c4cccc5c4C(C)(C)c4ccccc4-5)c3[2H])c([2H])c1C2(C([2H])([2H])[2H])C([2H])([2H])[2H]]

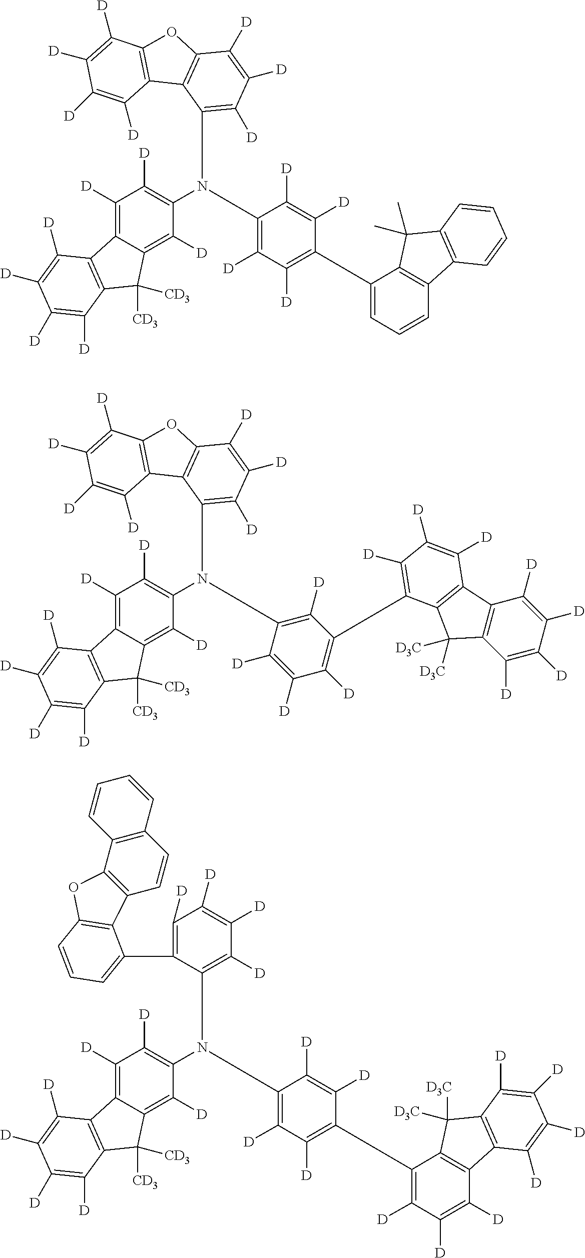

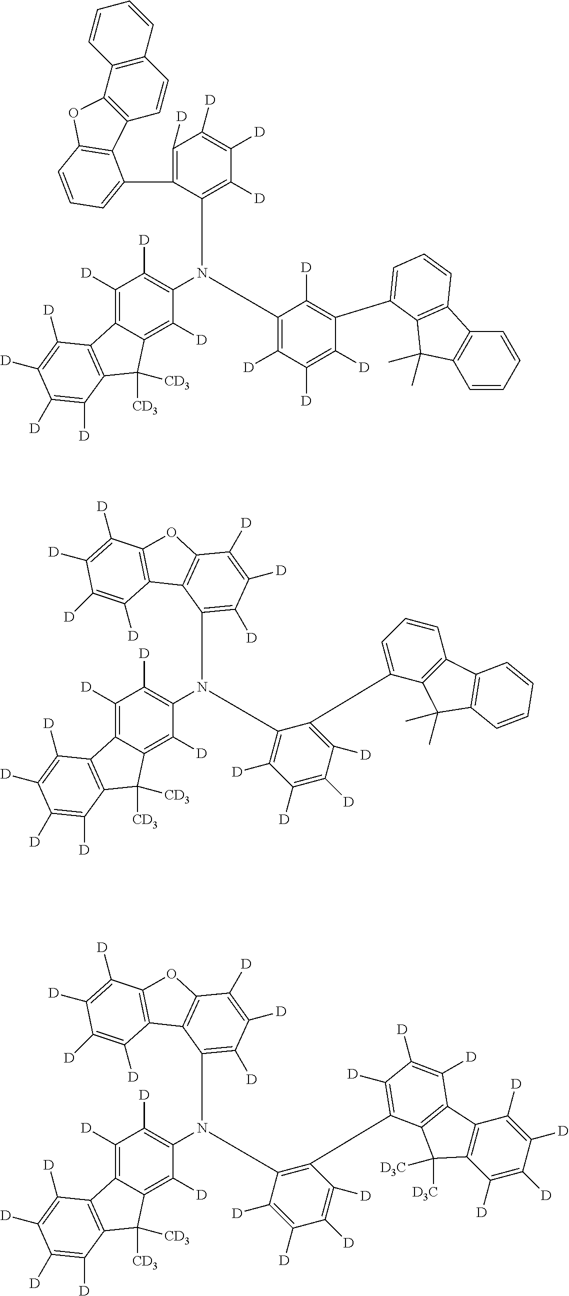

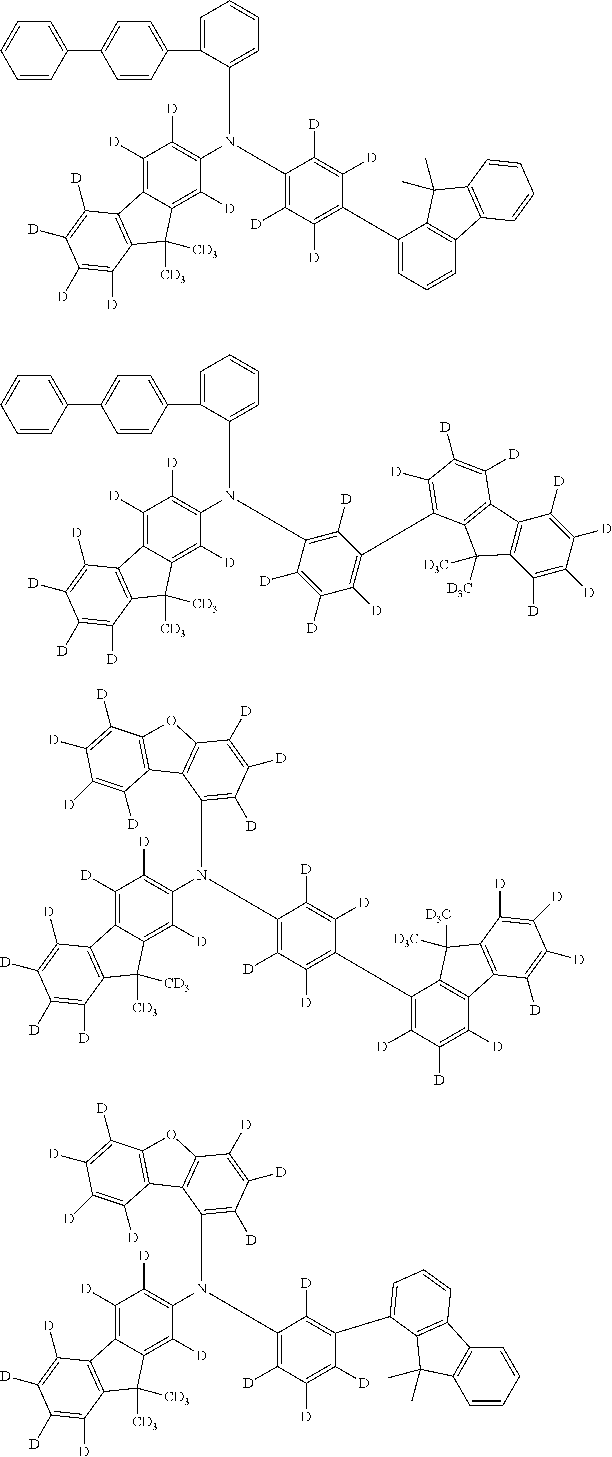

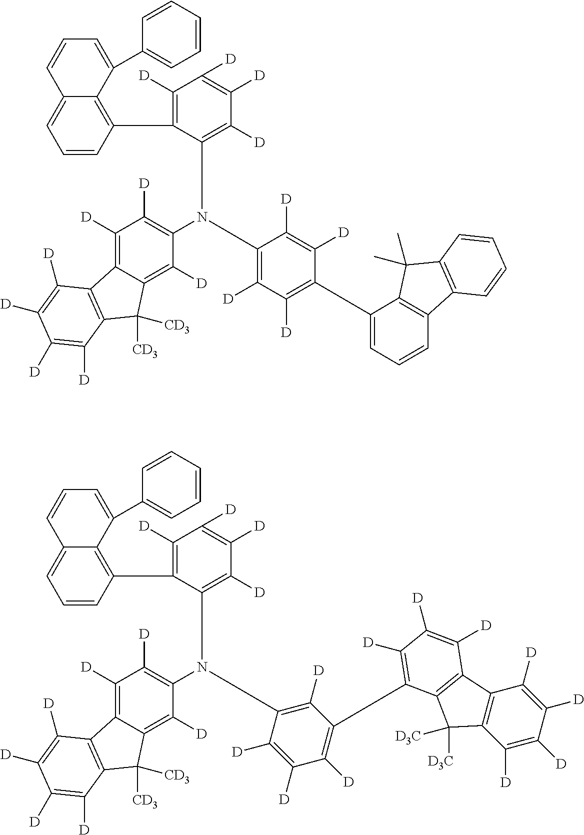

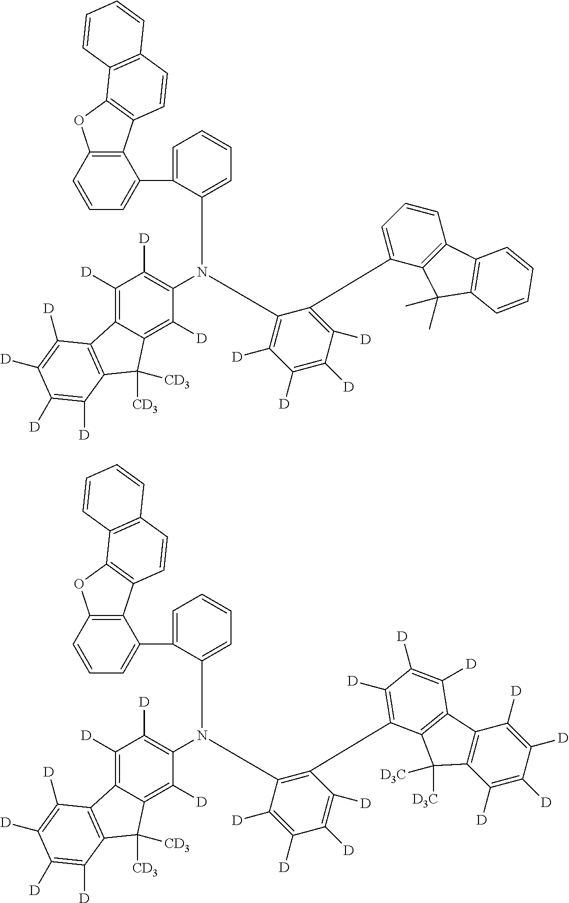

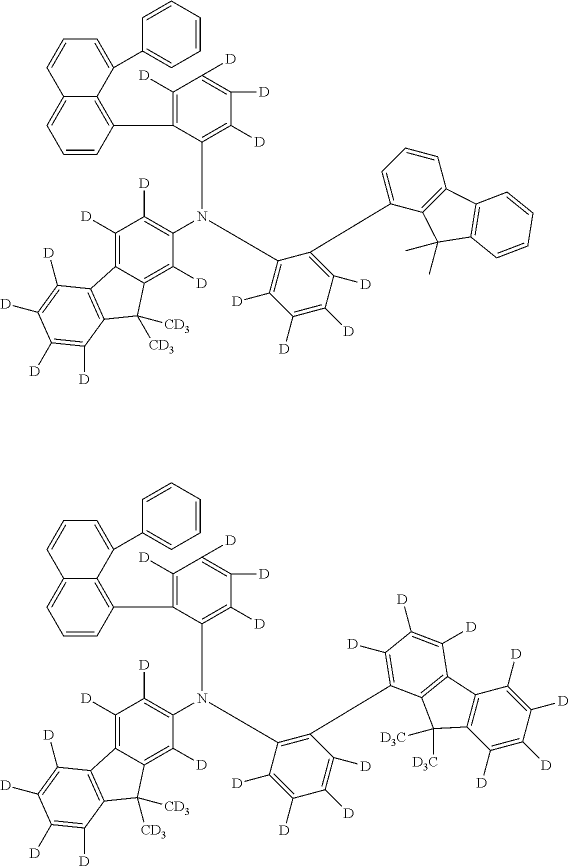

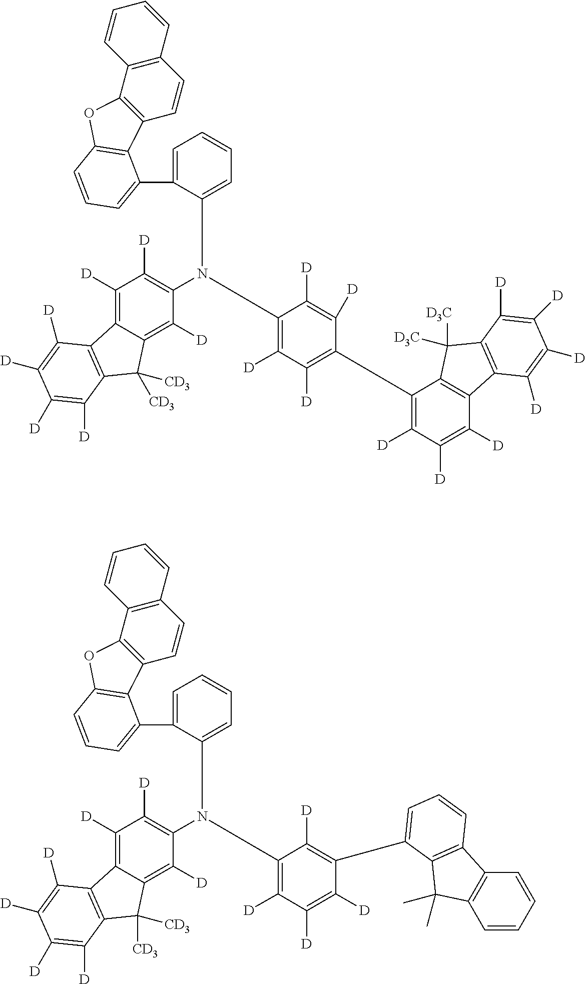 [SMILES: [2H]c1c([2H])c([2H])c2c(c1[2H])-c1c([2H])c([2H])c(N(c3ccccc3-c3cccc4oc5c6ccccc6ccc5c34)c3c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c([2H])c3[2H])c([2H])c1C2(C([2H])([2H])[2H])C([2H])([2H])[2H] ; [2H]c1c([2H])c([2H])c2c(c1[2H])-c1c([2H])c([2H])c(N(c3ccccc3-c3cccc4oc5c6ccccc6ccc5c34)c3c([2H])c([2H])c([2H])c(-c4cccc5c4C(C)(C)c4ccccc4-5)c3[2H])c([2H])c1C2(C([2H])([2H])[2H])C([2H])([2H])[2H]]

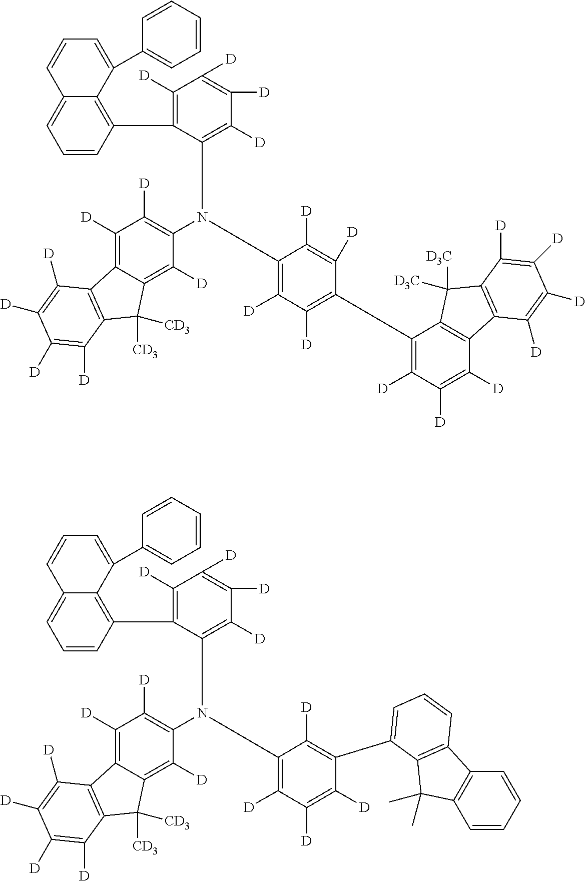

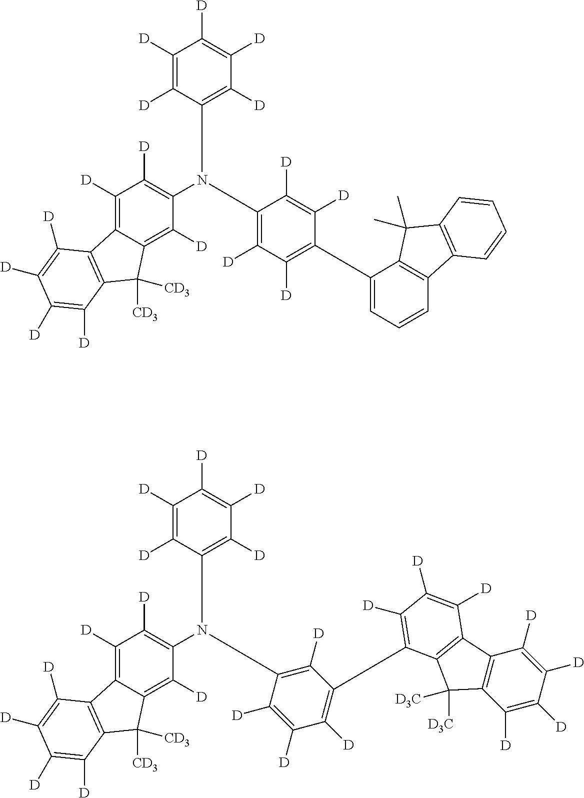 [SMILES: [2H]c1c([2H])c([2H])c(N(c2c([2H])c([2H])c(-c3cccc4c3C(C)(C)c3ccccc3-4)c([2H])c2[2H])c2c([2H])c([2H])c3c(c2[2H])C(C([2H])([2H])[2H])(C([2H])([2H])[2H])c2c([2H])c([2H])c([2H])c([2H])c2-3)c([2H])c1[2H] ; [2H]c1c([2H])c([2H])c(N(c2c([2H])c([2H])c([2H])c(-c3c([2H])c([2H])c([2H])c4c3C(C([2H])([2H])[2H])(C([2H])([2H])[2H])c3c([2H])c([2H])c([2H])c([2H])c3-4)c2[2H])c2c([2H])c([2H])c3c(c2[2H])C(C([2H])([2H])[2H])(C([2H])([2H])[2H])c2c([2H])c([2H])c([2H])c([2H])c2-3)c([2H])c1[2H]]

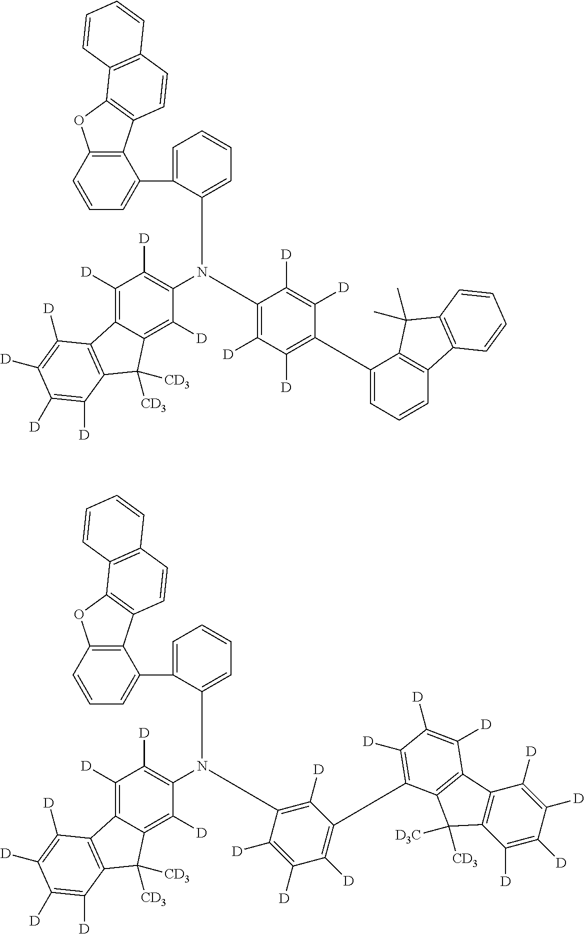 [SMILES: [2H]c1c([2H])c([2H])c2c(c1[2H])-c1c([2H])c([2H])c(N(c3ccccc3-c3cccc4oc5c6ccccc6ccc5c34)c3c([2H])c([2H])c(-c4cccc5c4C(C)(C)c4ccccc4-5)c([2H])c3[2H])c([2H])c1C2(C([2H])([2H])[2H])C([2H])([2H])[2H] ; [2H]c1c([2H])c([2H])c2c(c1[2H])-c1c([2H])c([2H])c(N(c3ccccc3-c3cccc4oc5c6ccccc6ccc5c34)c3c([2H])c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c3[2H])c([2H])c1C2(C([2H])([2H])[2H])C([2H])([2H])[2H]]

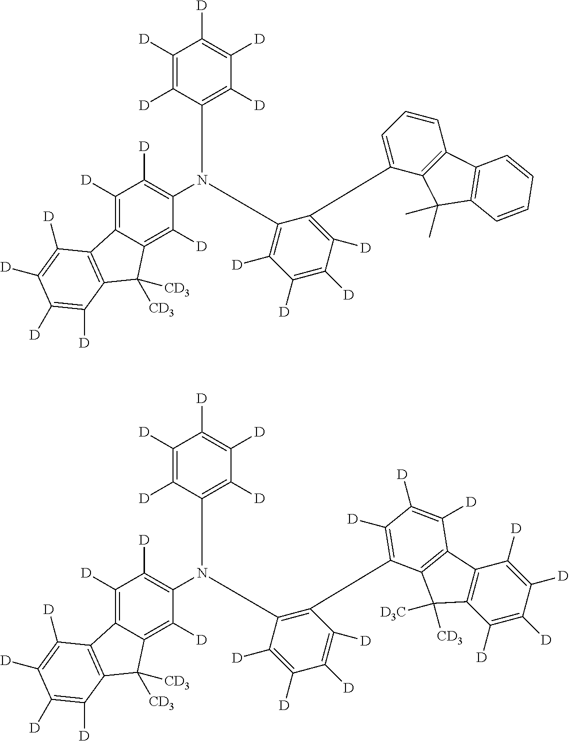 [SMILES: [2H]c1c([2H])c([2H])c(N(c2c([2H])c([2H])c3c(c2[2H])C(C([2H])([2H])[2H])(C([2H])([2H])[2H])c2c([2H])c([2H])c([2H])c([2H])c2-3)c2c([2H])c([2H])c([2H])c([2H])c2-c2cccc3c2C(C)(C)c2ccccc2-3)c([2H])c1[2H] ; [2H]c1c([2H])c([2H])c(N(c2c([2H])c([2H])c3c(c2[2H])C(C([2H])([2H])[2H])(C([2H])([2H])[2H])c2c([2H])c([2H])c([2H])c([2H])c2-3)c2c([2H])c([2H])c([2H])c([2H])c2-c2c([2H])c([2H])c([2H])c3c2C(C([2H])([2H])[2H])(C([2H])([2H])[2H])c2c([2H])c([2H])c([2H])c([2H])c2-3)c([2H])c1[2H]]

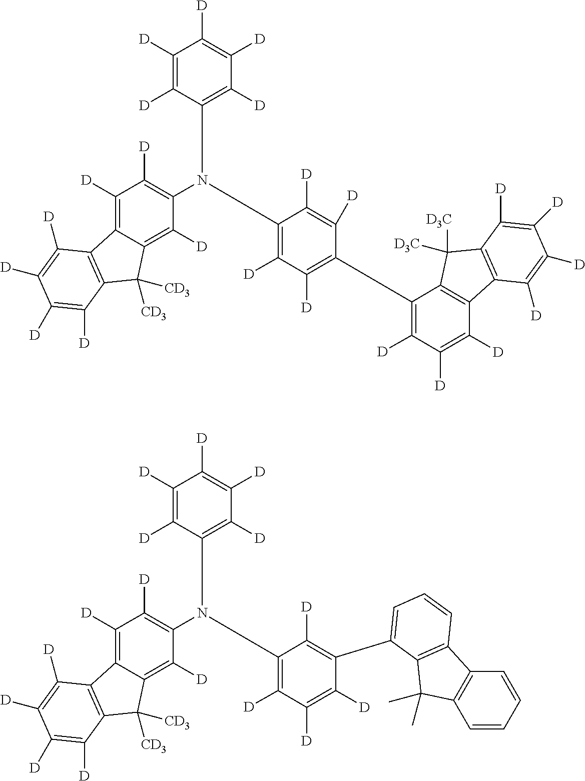 [SMILES: [2H]c1c([2H])c([2H])c(N(c2c([2H])c([2H])c(-c3c([2H])c([2H])c([2H])c4c3C(C([2H])([2H])[2H])(C([2H])([2H])[2H])c3c([2H])c([2H])c([2H])c([2H])c3-4)c([2H])c2[2H])c2c([2H])c([2H])c3c(c2[2H])C(C([2H])([2H])[2H])(C([2H])([2H])[2H])c2c([2H])c([2H])c([2H])c([2H])c2-3)c([2H])c1[2H] ; [2H]c1c([2H])c([2H])c(N(c2c([2H])c([2H])c([2H])c(-c3cccc4c3C(C)(C)c3ccccc3-4)c2[2H])c2c([2H])c([2H])c3c(c2[2H])C(C([2H])([2H])[2H])(C([2H])([2H])[2H])c2c([2H])c([2H])c([2H])c([2H])c2-3)c([2H])c1[2H]]

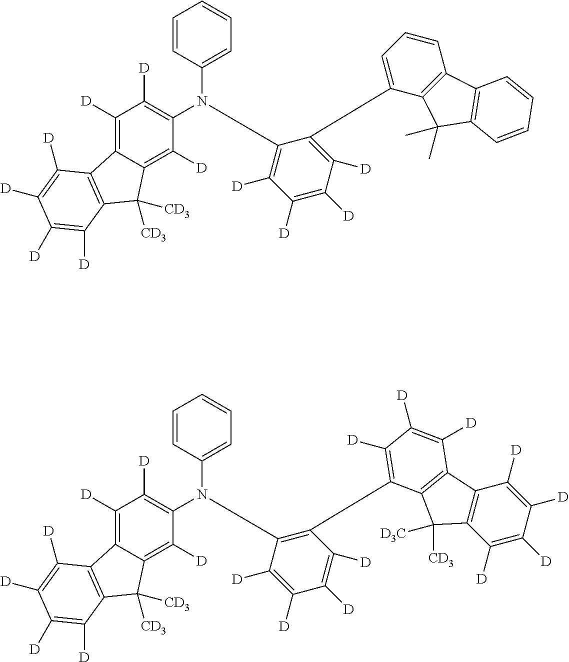 [SMILES: [2H]c1c([2H])c([2H])c(N(c2ccccc2)c2c([2H])c([2H])c3c(c2[2H])C(C([2H])([2H])[2H])(C([2H])([2H])[2H])c2c([2H])c([2H])c([2H])c([2H])c2-3)c(-c2cccc3c2C(C)(C)c2ccccc2-3)c1[2H] ; [2H]c1c([2H])c([2H])c(N(c2ccccc2)c2c([2H])c([2H])c3c(c2[2H])C(C([2H])([2H])[2H])(C([2H])([2H])[2H])c2c([2H])c([2H])c([2H])c([2H])c2-3)c(-c2c([2H])c([2H])c([2H])c3c2C(C([2H])([2H])[2H])(C([2H])([2H])[2H])c2c([2H])c([2H])c([2H])c([2H])c2-3)c1[2H]]

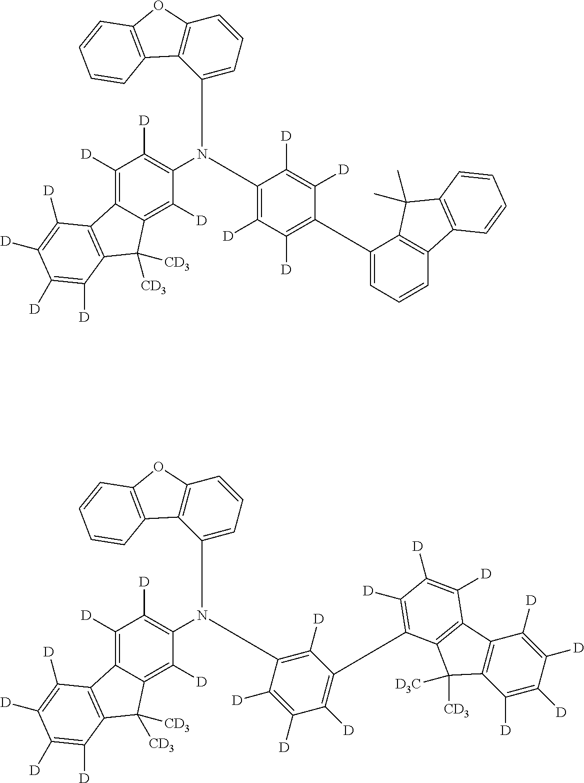 [SMILES: [2H]c1c([2H])c([2H])c2c(c1[2H])-c1c([2H])c([2H])c(N(c3c([2H])c([2H])c(-c4cccc5c4C(C)(C)c4ccccc4-5)c([2H])c3[2H])c3cccc4oc5ccccc5c34)c([2H])c1C2(C([2H])([2H])[2H])C([2H])([2H])[2H] ; [2H]c1c([2H])c([2H])c2c(c1[2H])-c1c([2H])c([2H])c(N(c3c([2H])c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c3[2H])c3cccc4oc5ccccc5c34)c([2H])c1C2(C([2H])([2H])[2H])C([2H])([2H])[2H]]

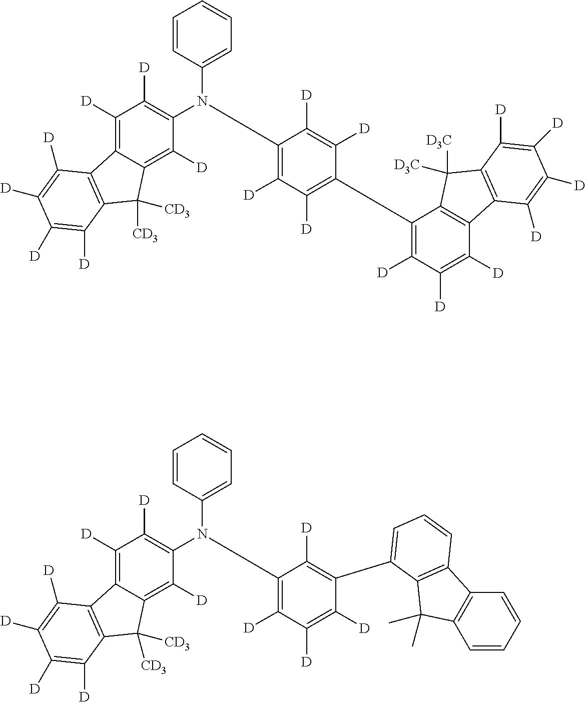 [SMILES: [2H]c1c([2H])c([2H])c2c(c1[2H])-c1c([2H])c([2H])c(N(c3ccccc3)c3c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c([2H])c3[2H])c([2H])c1C2(C([2H])([2H])[2H])C([2H])([2H])[2H] ; [2H]c1c([2H])c([2H])c2c(c1[2H])-c1c([2H])c([2H])c(N(c3ccccc3)c3c([2H])c([2H])c([2H])c(-c4cccc5c4C(C)(C)c4ccccc4-5)c3[2H])c([2H])c1C2(C([2H])([2H])[2H])C([2H])([2H])[2H]]

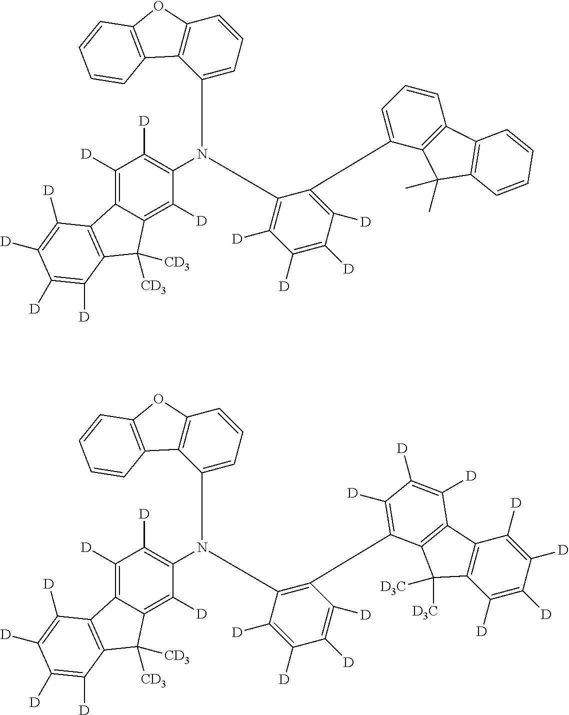 [SMILES: [2H]c1c([2H])c([2H])c(N(c2c([2H])c([2H])c3c(c2[2H])C(C([2H])([2H])[2H])(C([2H])([2H])[2H])c2c([2H])c([2H])c([2H])c([2H])c2-3)c2cccc3oc4ccccc4c23)c(-c2cccc3c2C(C)(C)c2ccccc2-3)c1[2H] ; [2H]c1c([2H])c([2H])c(N(c2c([2H])c([2H])c3c(c2[2H])C(C([2H])([2H])[2H])(C([2H])([2H])[2H])c2c([2H])c([2H])c([2H])c([2H])c2-3)c2cccc3oc4ccccc4c23)c(-c2c([2H])c([2H])c([2H])c3c2C(C([2H])([2H])[2H])(C([2H])([2H])[2H])c2c([2H])c([2H])c([2H])c([2H])c2-3)c1[2H]]

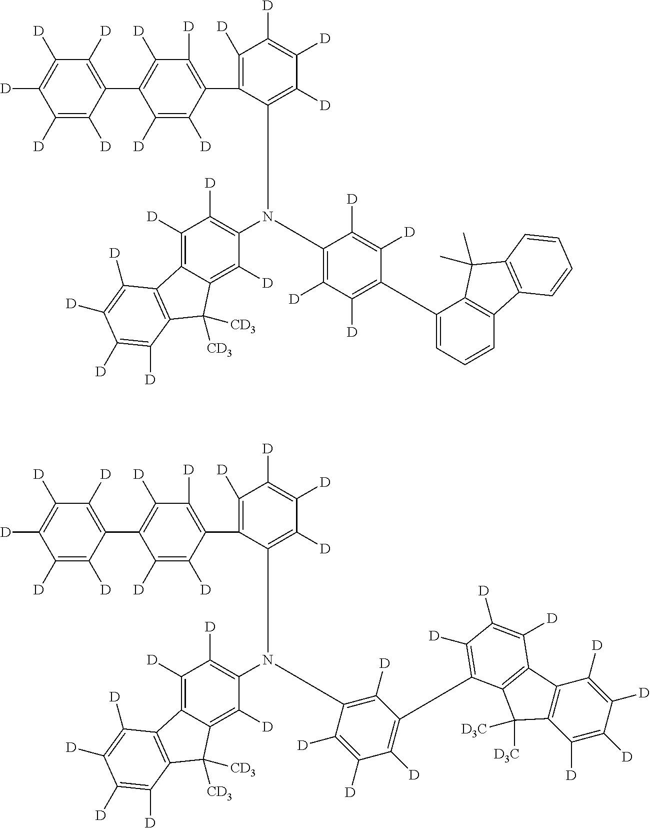

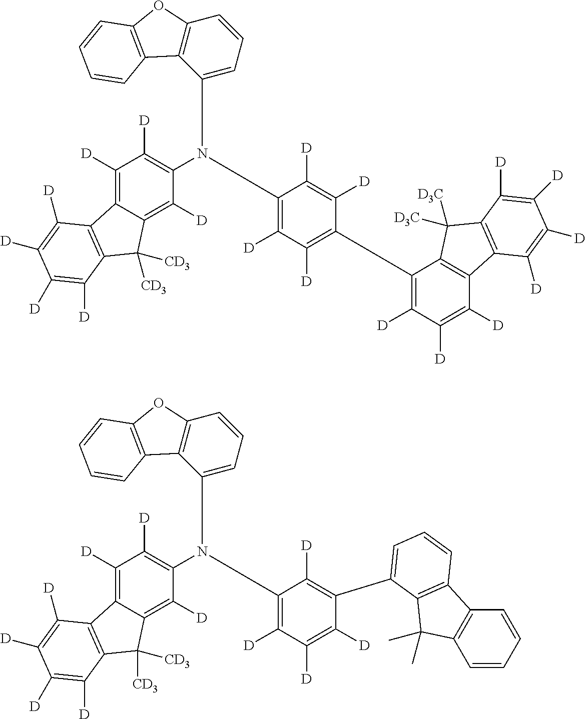 [SMILES: [2H]c1c([2H])c([2H])c2c(c1[2H])-c1c([2H])c([2H])c(N(c3c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c([2H])c3[2H])c3cccc4oc5ccccc5c34)c([2H])c1C2(C([2H])([2H])[2H])C([2H])([2H])[2H] ; [2H]c1c([2H])c([2H])c2c(c1[2H])-c1c([2H])c([2H])c(N(c3c([2H])c([2H])c([2H])c(-c4cccc5c4C(C)(C)c4ccccc4-5)c3[2H])c3cccc4oc5ccccc5c34)c([2H])c1C2(C([2H])([2H])[2H])C([2H])([2H])[2H]]

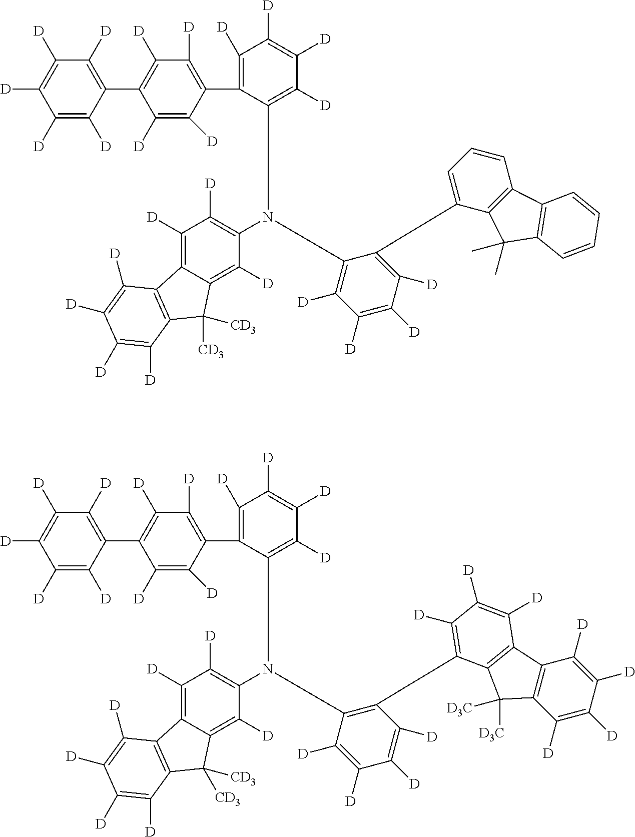

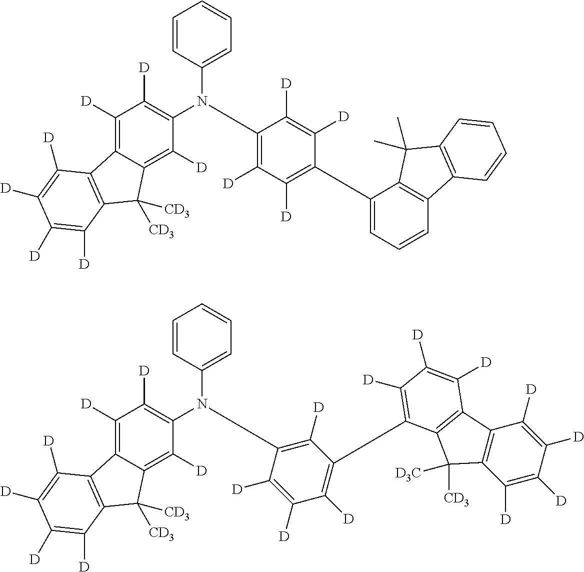 [SMILES: [2H]c1c([2H])c([2H])c2c(c1[2H])-c1c([2H])c([2H])c(N(c3ccccc3)c3c([2H])c([2H])c(-c4cccc5c4C(C)(C)c4ccccc4-5)c([2H])c3[2H])c([2H])c1C2(C([2H])([2H])[2H])C([2H])([2H])[2H] ; [2H]c1c([2H])c([2H])c2c(c1[2H])-c1c([2H])c([2H])c(N(c3ccccc3)c3c([2H])c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c3[2H])c([2H])c1C2(C([2H])([2H])[2H])C([2H])([2H])[2H]]

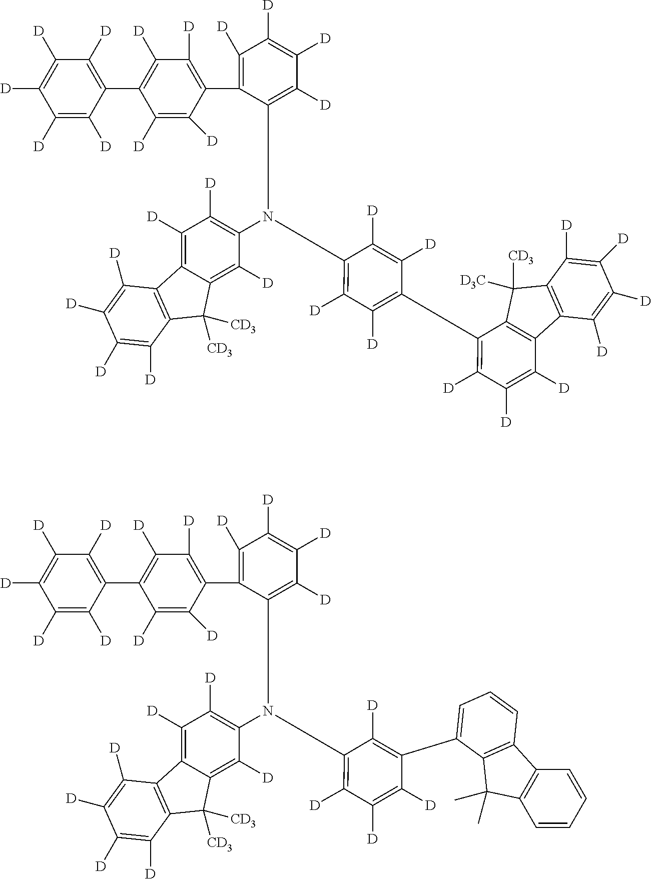

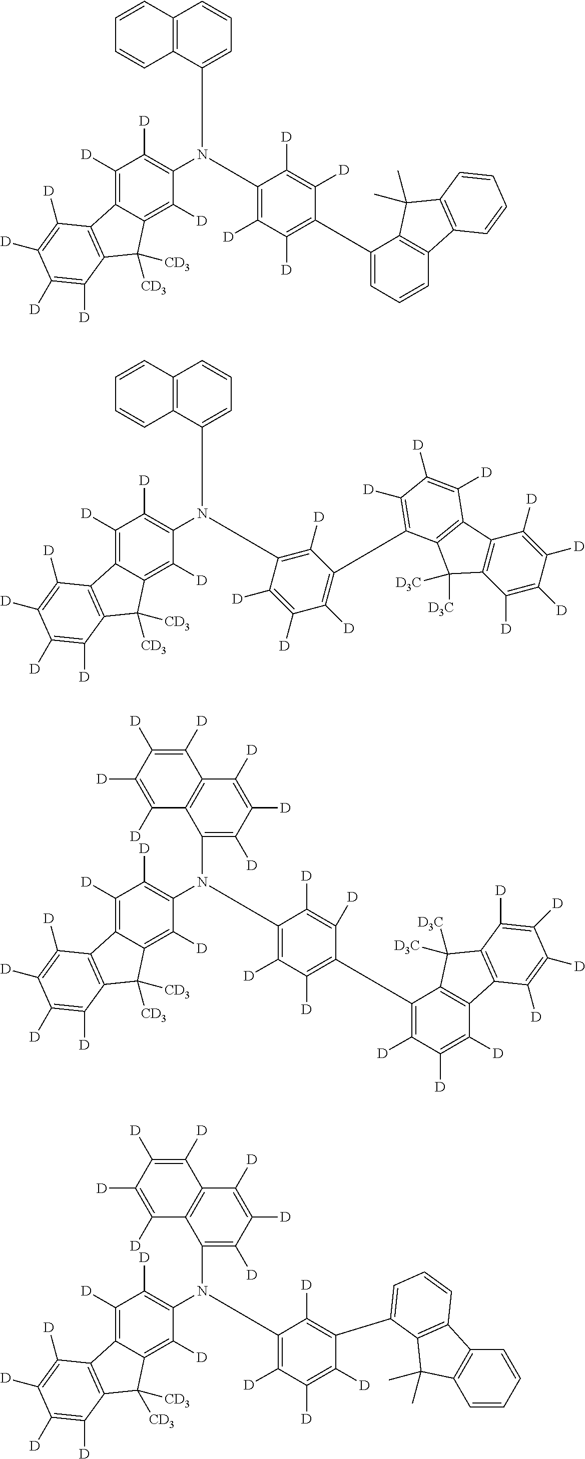

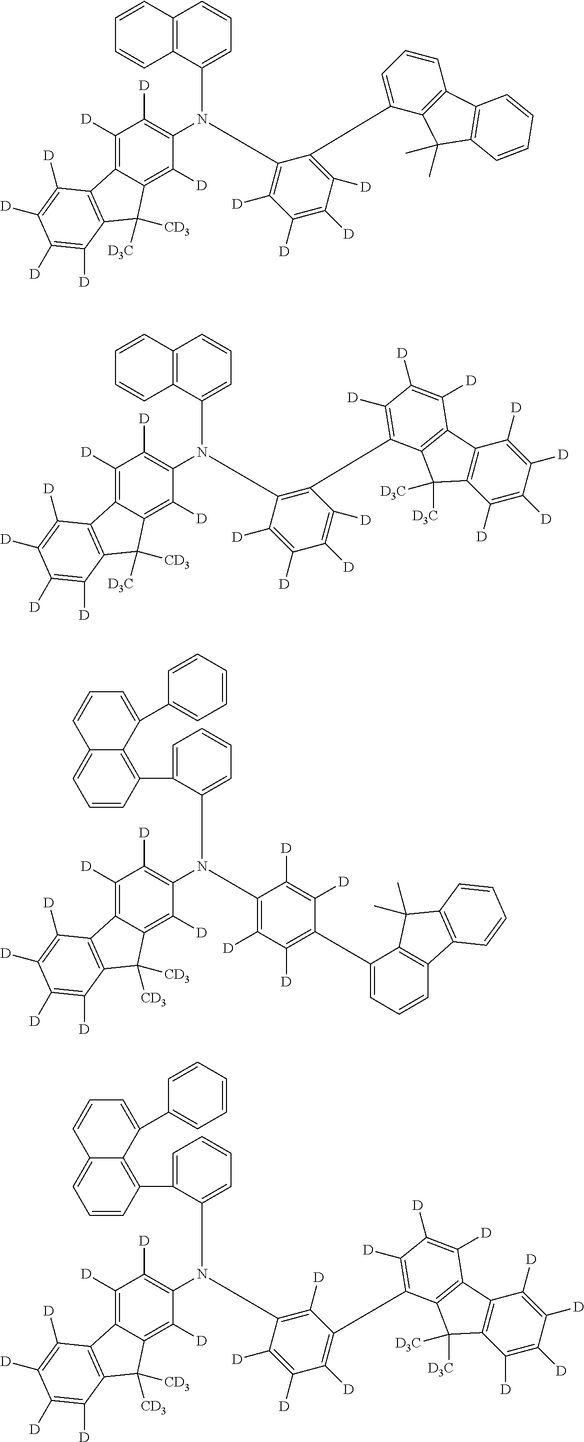

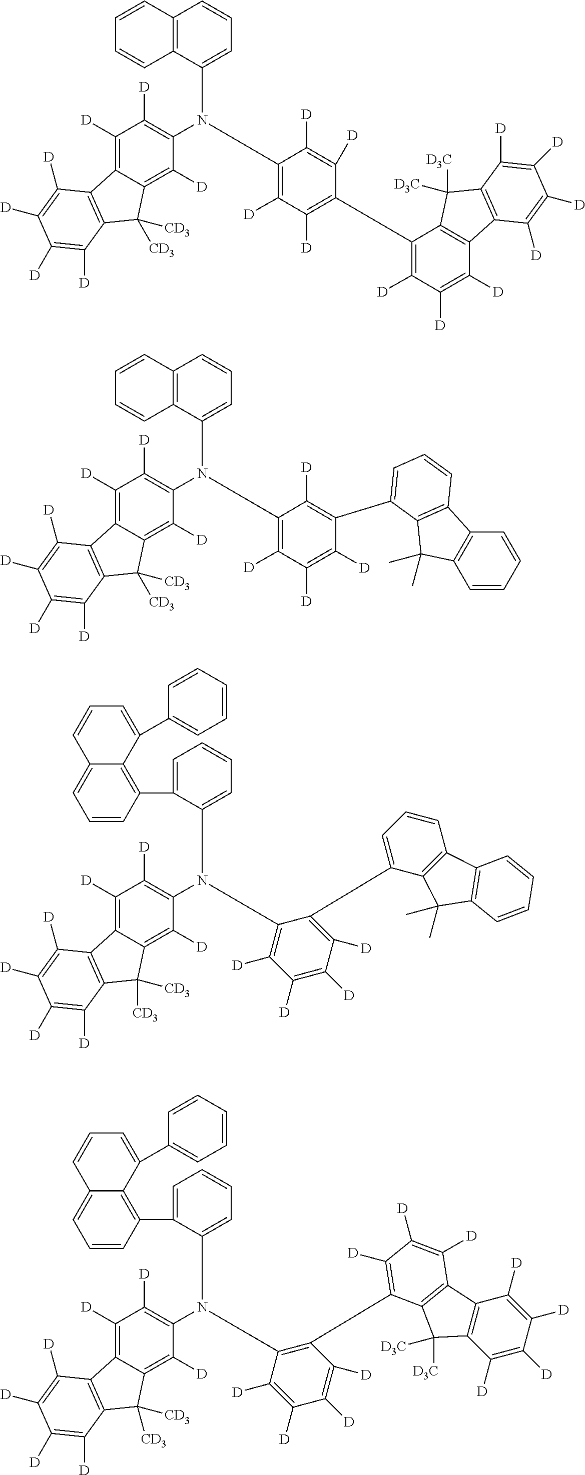

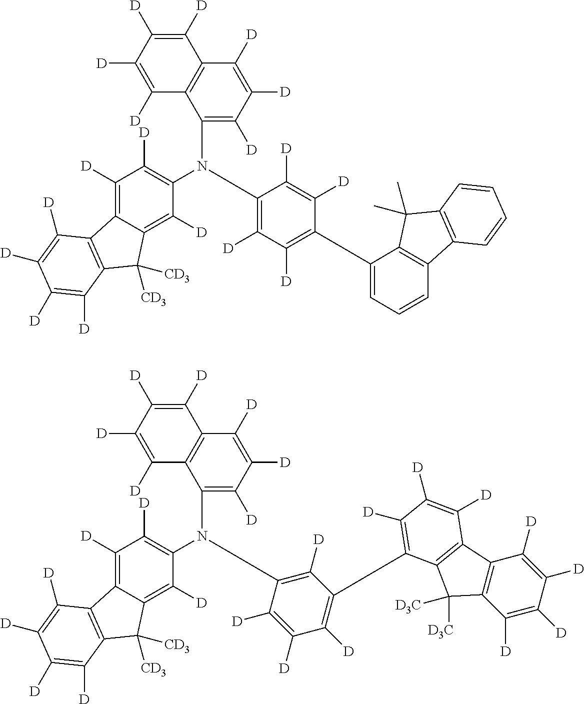

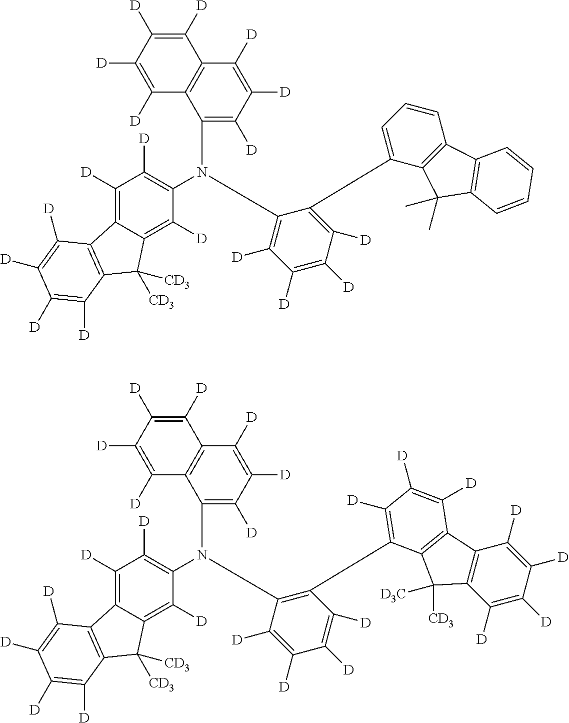

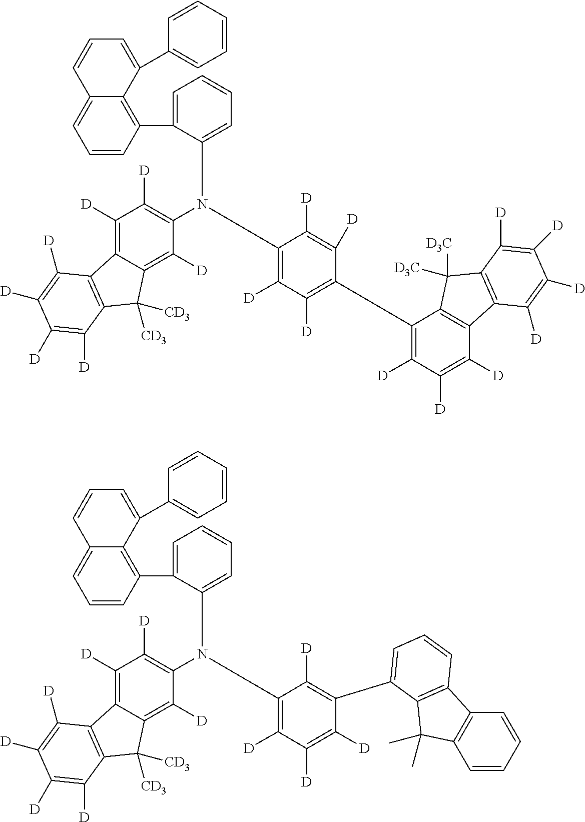 [SMILES: [2H]c1c([2H])c([2H])c2c(c1[2H])-c1c([2H])c([2H])c(N(c3ccccc3-c3cccc4cccc(-c5ccccc5)c34)c3c([2H])c([2H])c(-c4c([2H])c([2H])c([2H])c5c4C(C([2H])([2H])[2H])(C([2H])([2H])[2H])c4c([2H])c([2H])c([2H])c([2H])c4-5)c([2H])c3[2H])c([2H])c1C2(C([2H])([2H])[2H])C([2H])([2H])[2H] ; [2H]c1c([2H])c([2H])c2c(c1[2H])-c1c([2H])c([2H])c(N(c3ccccc3-c3cccc4cccc(-c5ccccc5)c34)c3c([2H])c([2H])c([2H])c(-c4cccc5c4C(C)(C)c4ccccc4-5)c3[2H])c([2H])c1C2(C([2H])([2H])[2H])C([2H])([2H])[2H]]

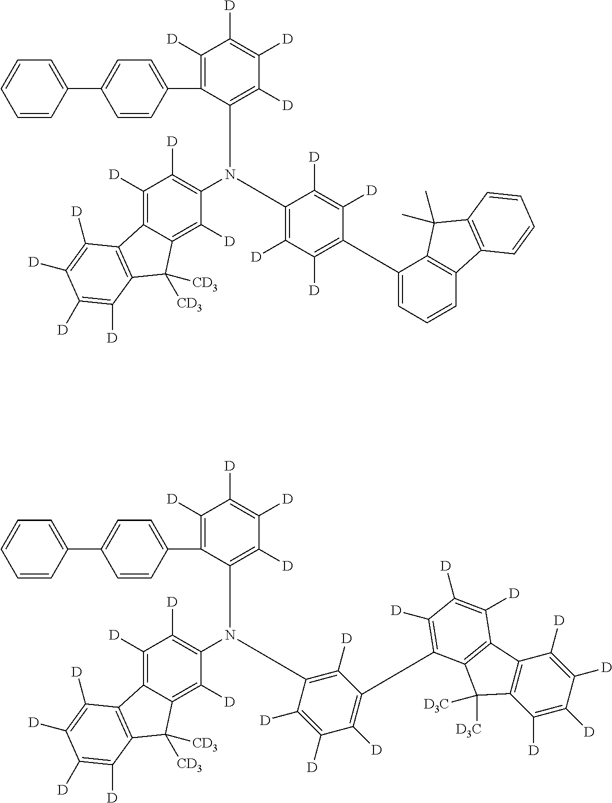

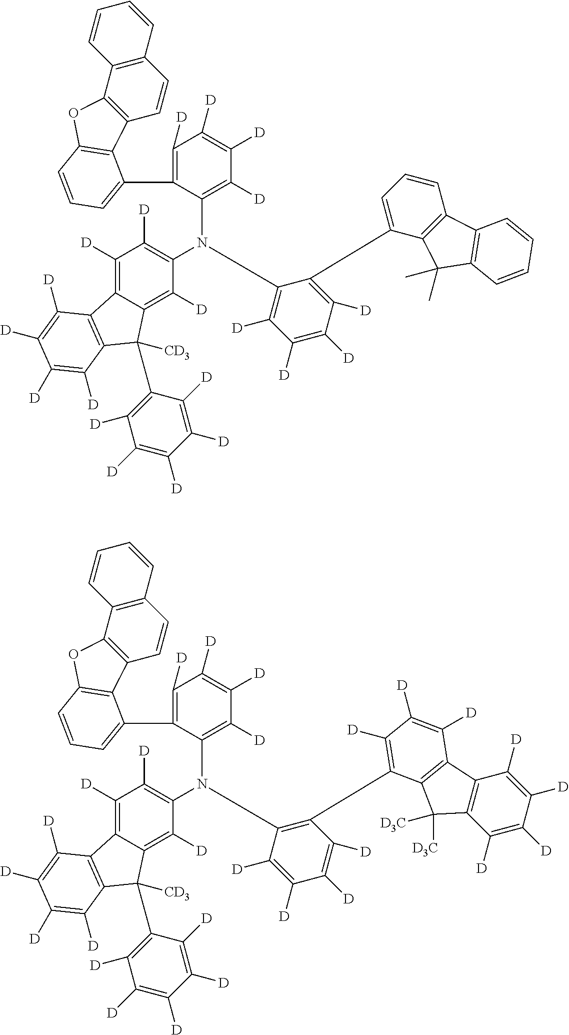

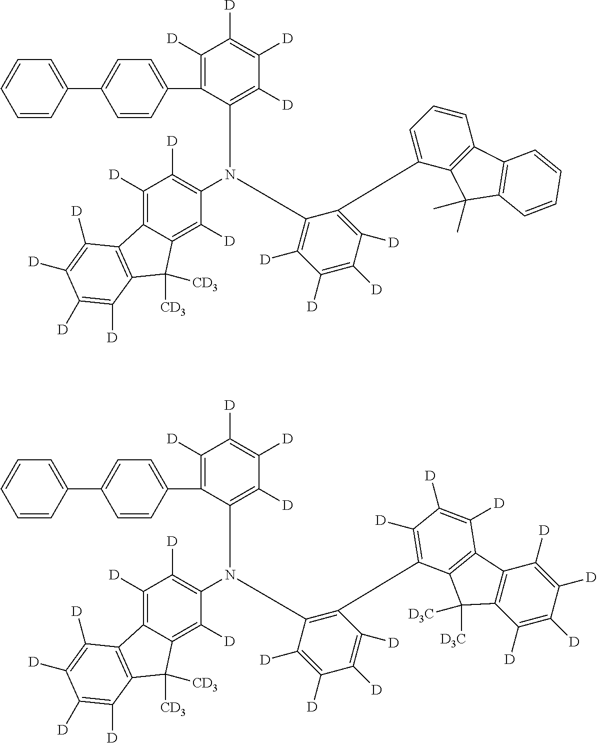

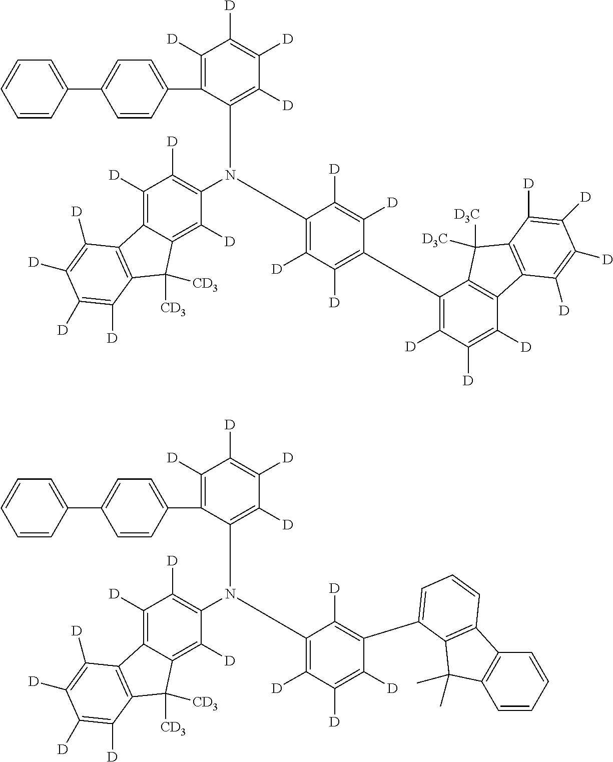

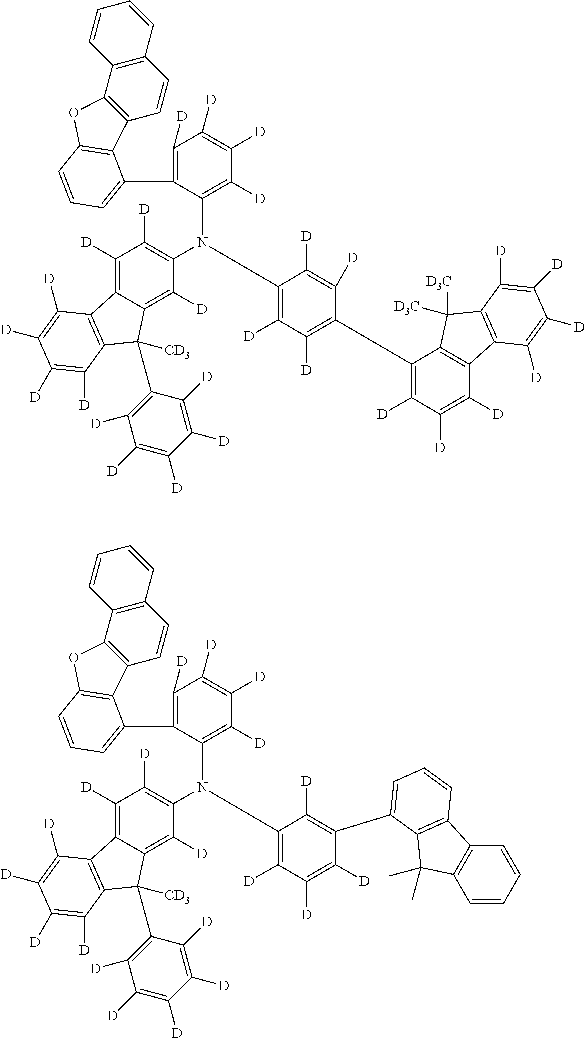

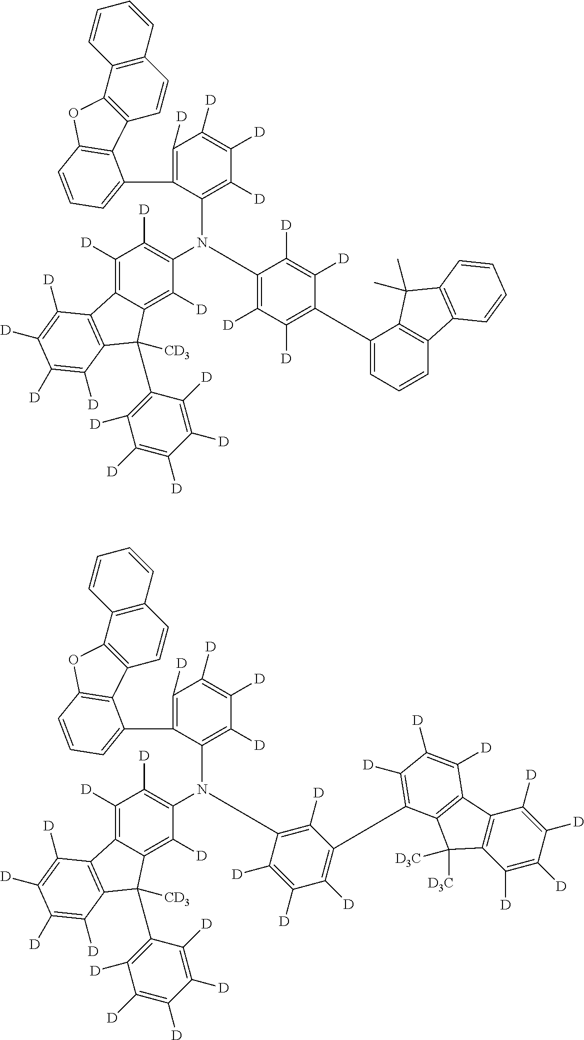

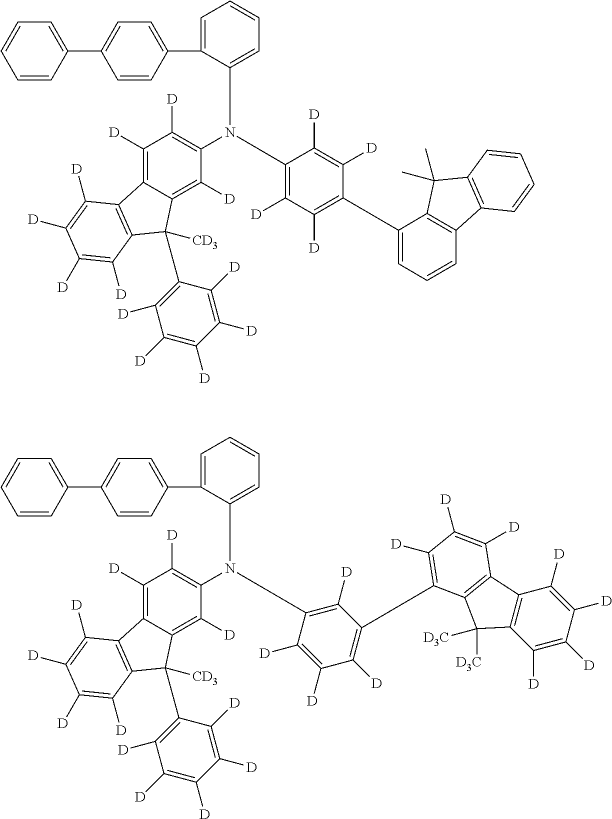 [SMILES: [2H]c1c([2H])c([2H])c(C2(C([2H])([2H])[2H])c3c([2H])c([2H])c([2H])c([2H])c3-c3c([2H])c([2H])c(N(c4ccccc4-c4ccc(-c5ccccc5)cc4)c4c([2H])c([2H])c(-c5cccc6c5C(C)(C)c5ccccc5-6)c([2H])c4[2H])c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4ccccc4-c4ccc(-c5ccccc5)cc4)c4c([2H])c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c4[2H])c([2H])c32)c([2H])c1[2H]]

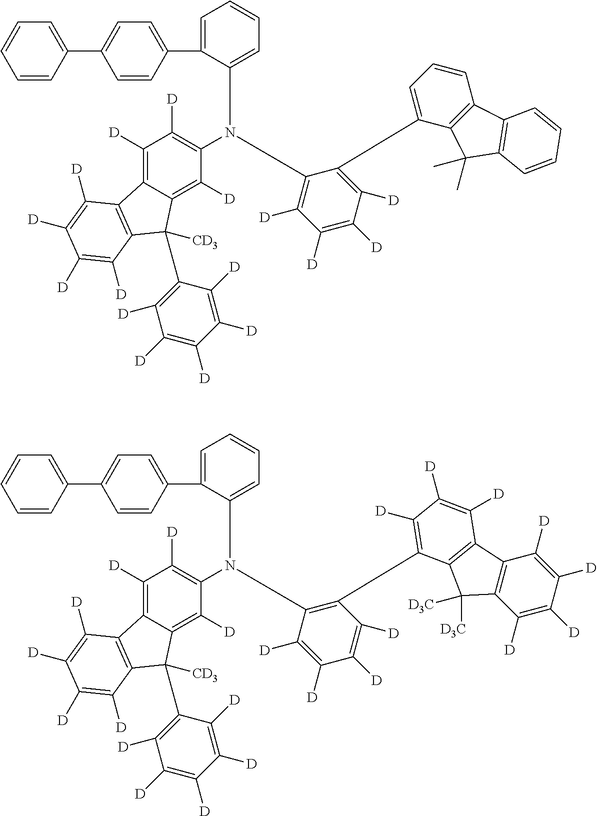 [SMILES: [2H]c1c([2H])c([2H])c(C2(C([2H])([2H])[2H])c3c([2H])c([2H])c([2H])c([2H])c3-c3c([2H])c([2H])c(N(c4ccccc4-c4ccc(-c5ccccc5)cc4)c4c([2H])c([2H])c([2H])c([2H])c4-c4cccc5c4C(C)(C)c4ccccc4-5)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4ccccc4-c4ccc(-c5ccccc5)cc4)c4c([2H])c([2H])c([2H])c([2H])c4-c4c([2H])c([2H])c([2H])c5c4C(C([2H])([2H])[2H])(C([2H])([2H])[2H])c4c([2H])c([2H])c([2H])c([2H])c4-5)c([2H])c32)c([2H])c1[2H]]

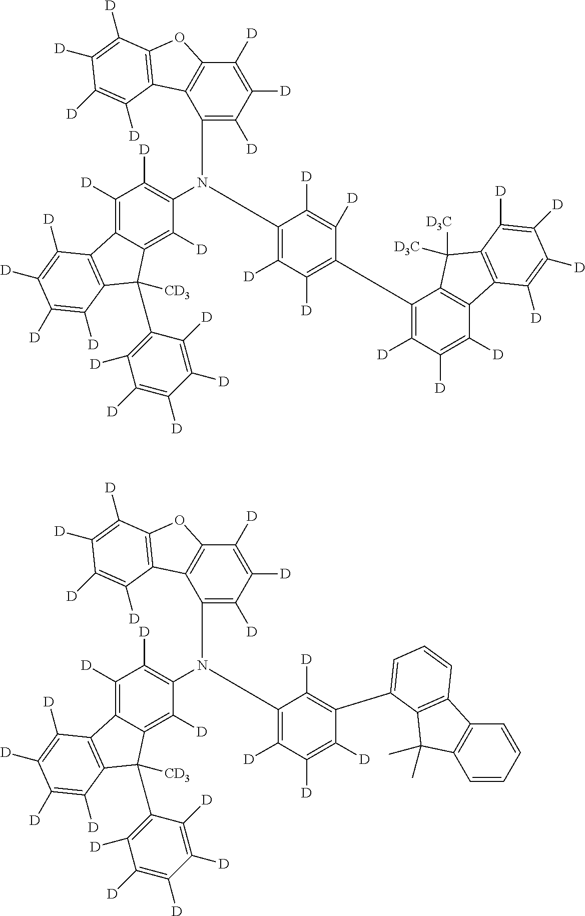

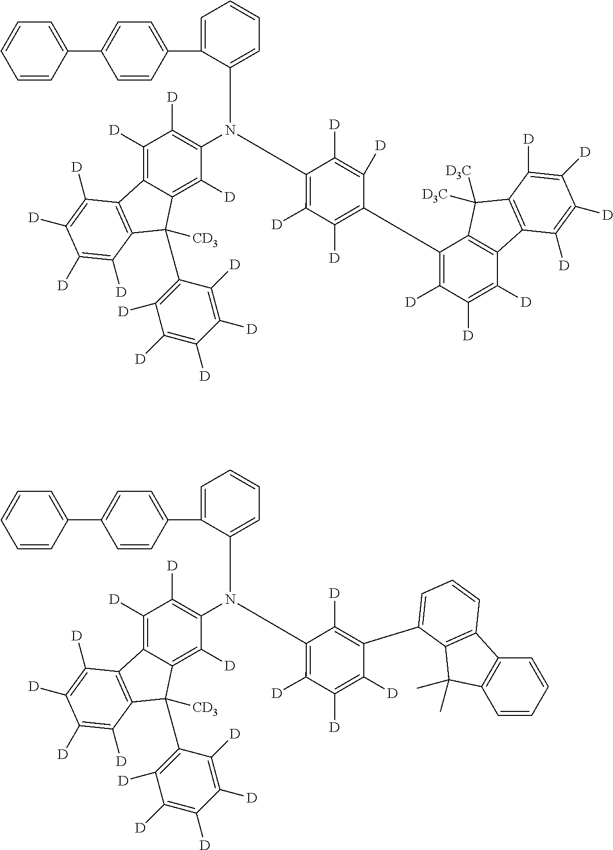 [SMILES: [2H]c1c([2H])c([2H])c(C2(C([2H])([2H])[2H])c3c([2H])c([2H])c([2H])c([2H])c3-c3c([2H])c([2H])c(N(c4ccccc4-c4ccc(-c5ccccc5)cc4)c4c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c([2H])c4[2H])c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4ccccc4-c4ccc(-c5ccccc5)cc4)c4c([2H])c([2H])c([2H])c(-c5cccc6c5C(C)(C)c5ccccc5-6)c4[2H])c([2H])c32)c([2H])c1[2H]]

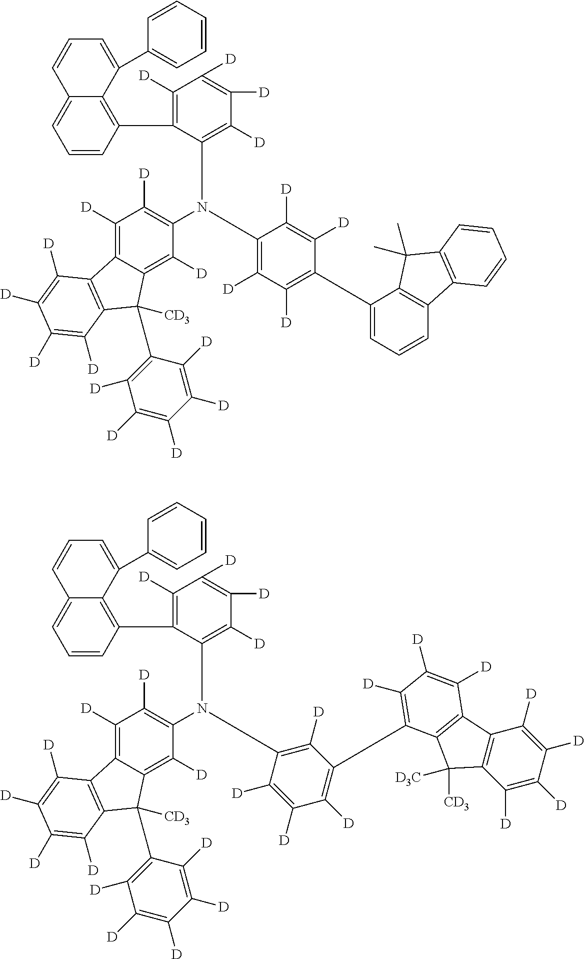

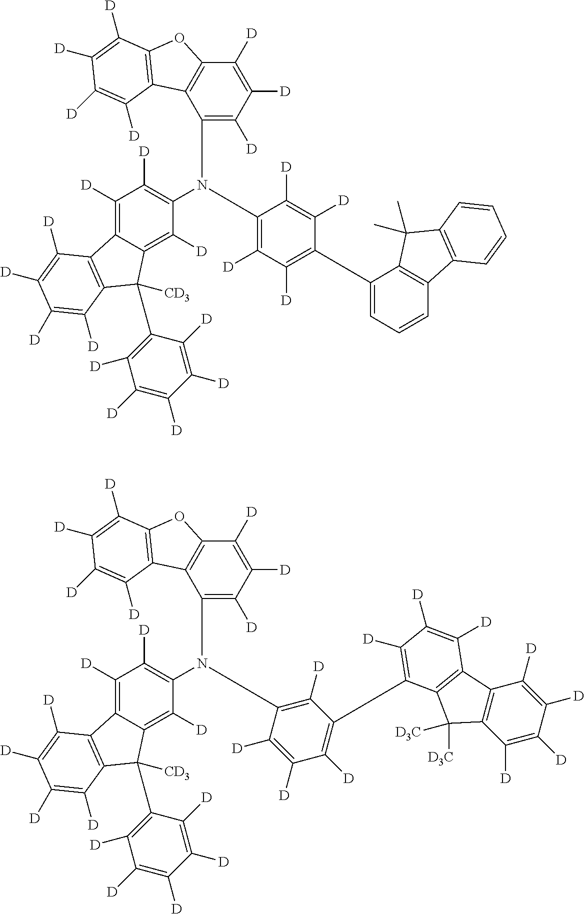

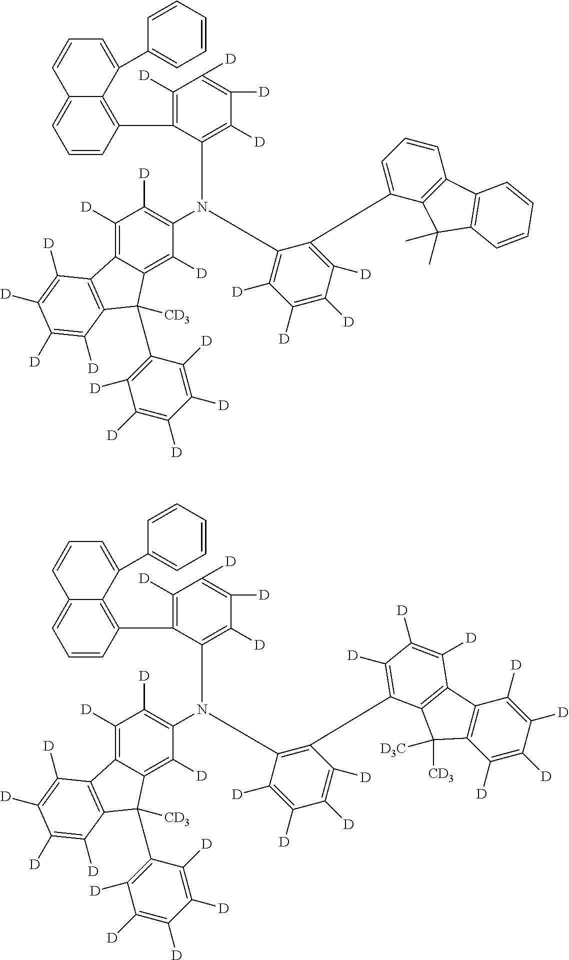

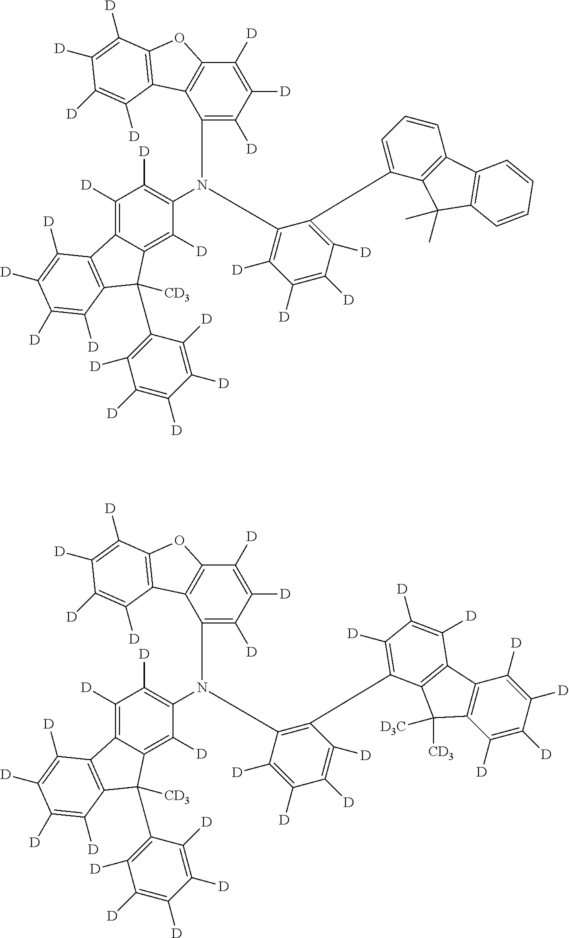

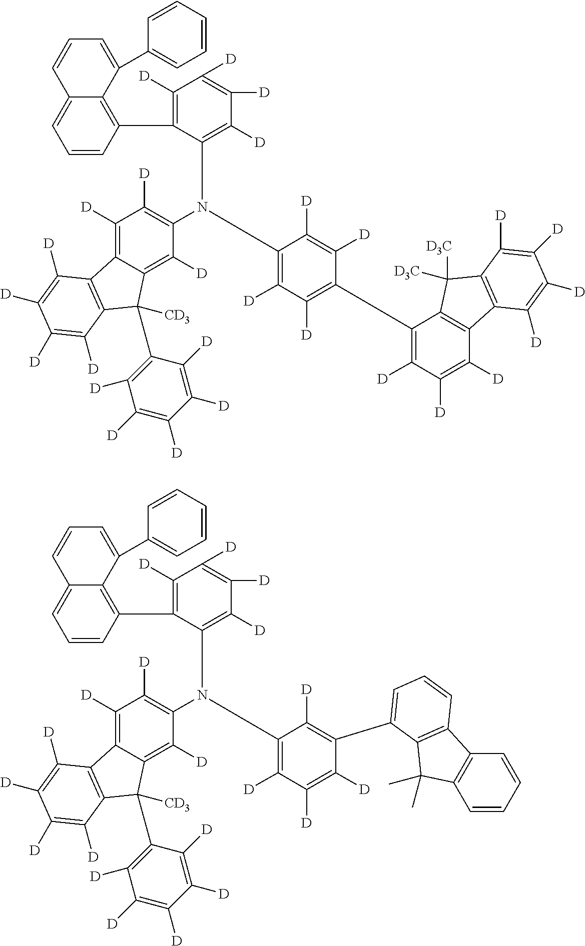

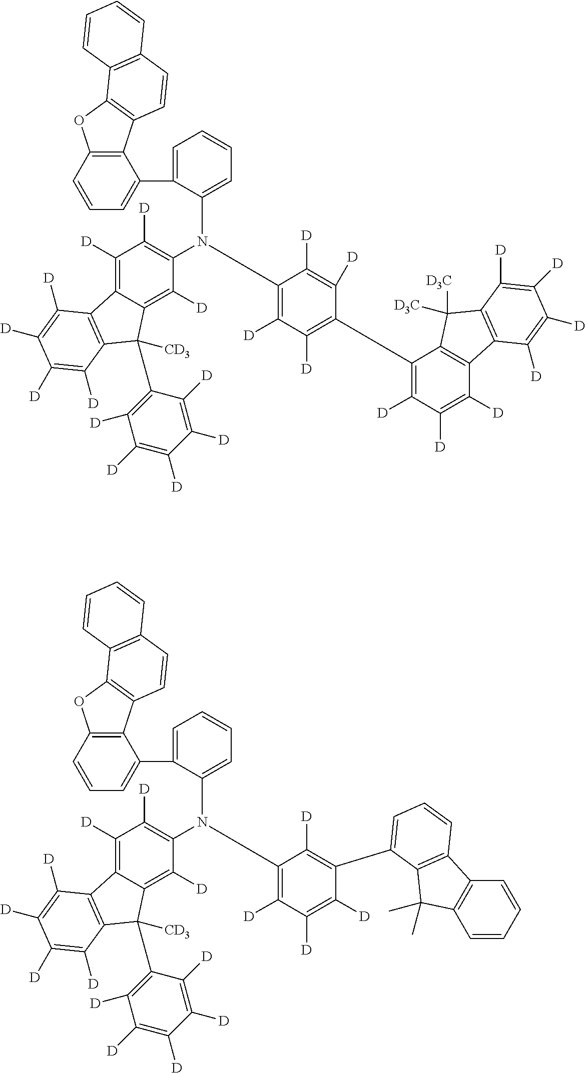

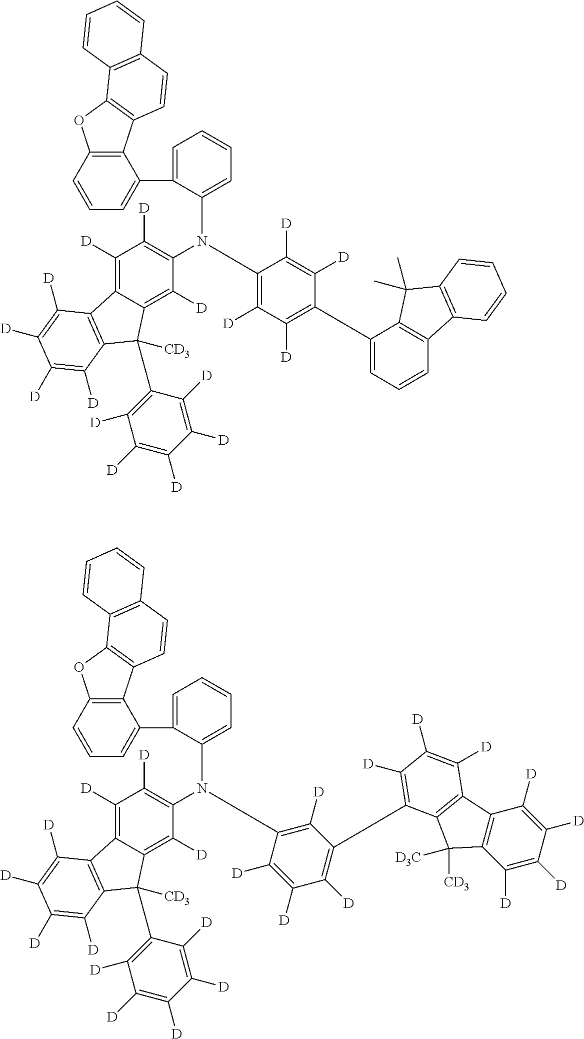

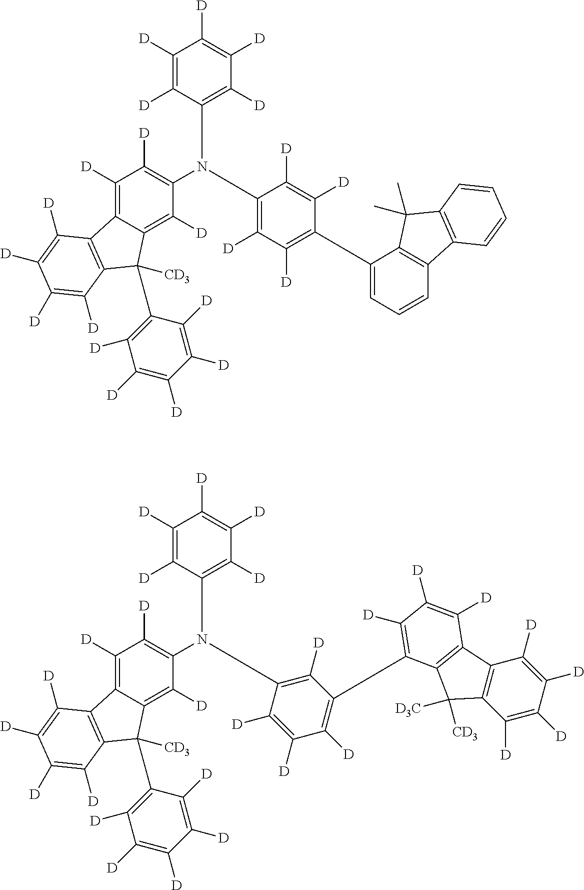

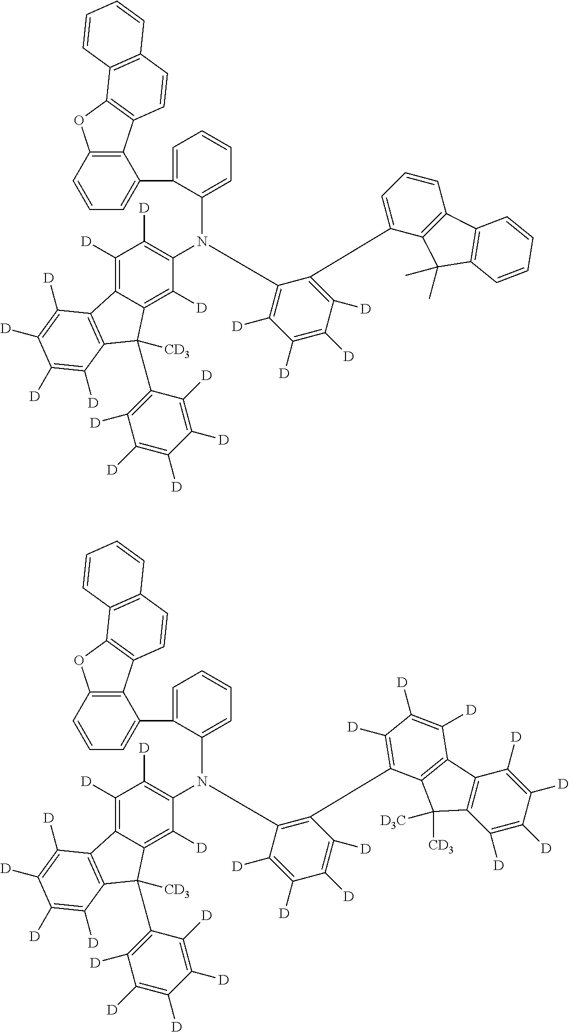

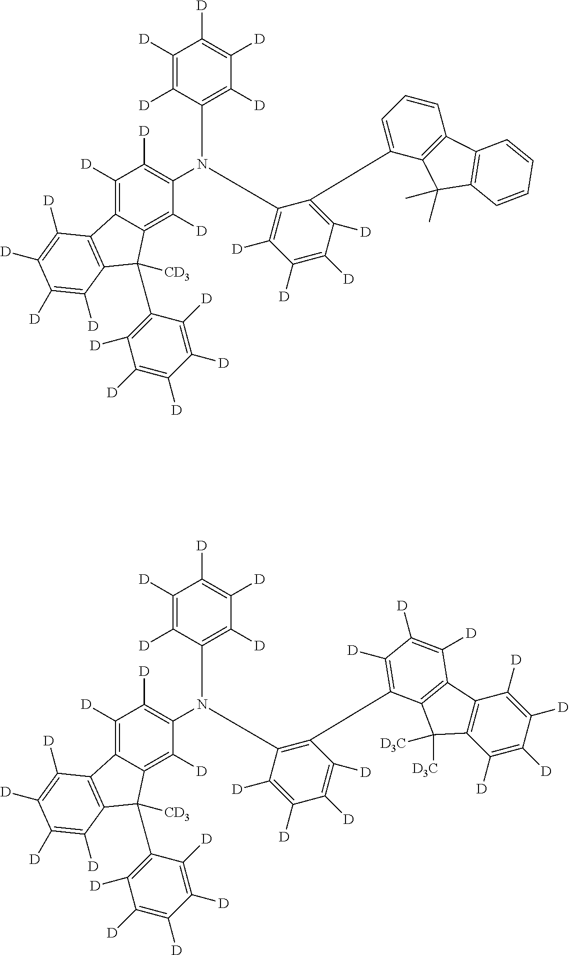

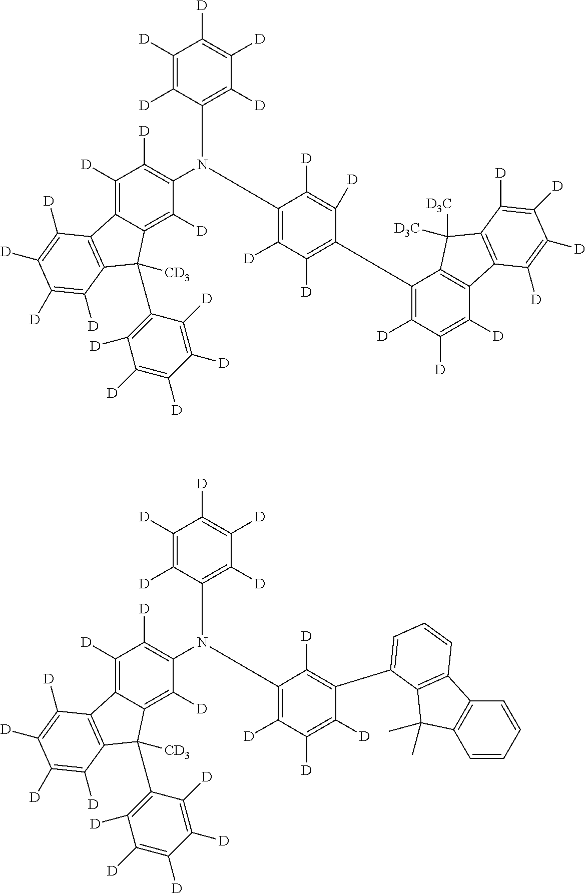

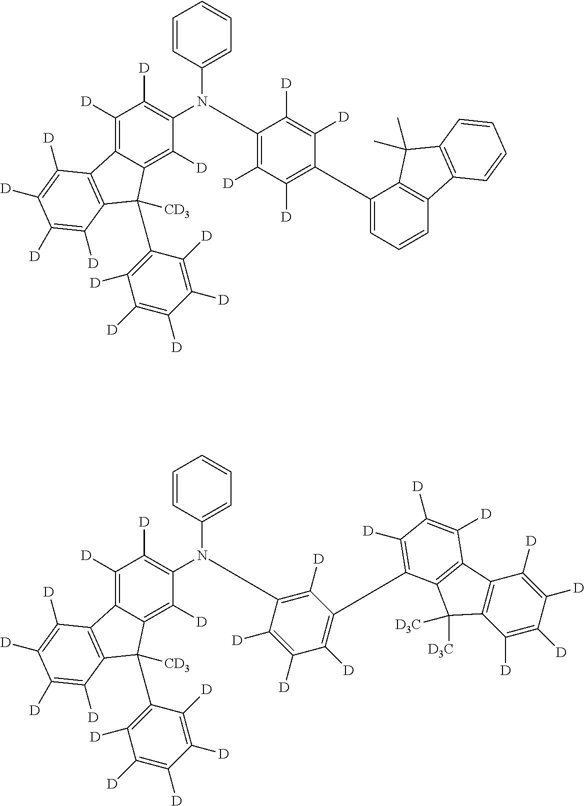 [SMILES: [2H]c1c([2H])c([2H])c(C2(C([2H])([2H])[2H])c3c([2H])c([2H])c([2H])c([2H])c3-c3c([2H])c([2H])c(N(c4ccccc4)c4c([2H])c([2H])c(-c5cccc6c5C(C)(C)c5ccccc5-6)c([2H])c4[2H])c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4ccccc4)c4c([2H])c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c4[2H])c([2H])c32)c([2H])c1[2H]]

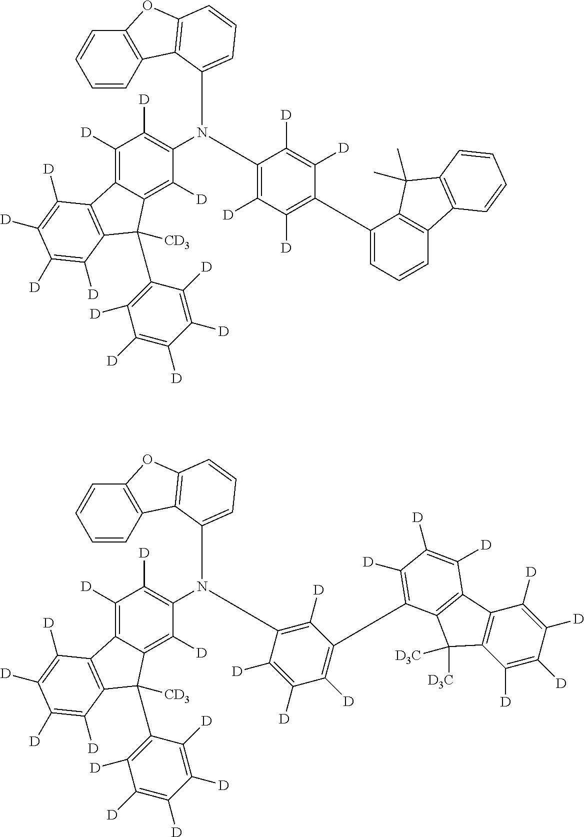 [SMILES: [2H]c1c([2H])c([2H])c(C2(C([2H])([2H])[2H])c3c([2H])c([2H])c([2H])c([2H])c3-c3c([2H])c([2H])c(N(c4c([2H])c([2H])c(-c5cccc6c5C(C)(C)c5ccccc5-6)c([2H])c4[2H])c4cccc5oc6ccccc6c45)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c4[2H])c4cccc5oc6ccccc6c45)c([2H])c32)c([2H])c1[2H]]

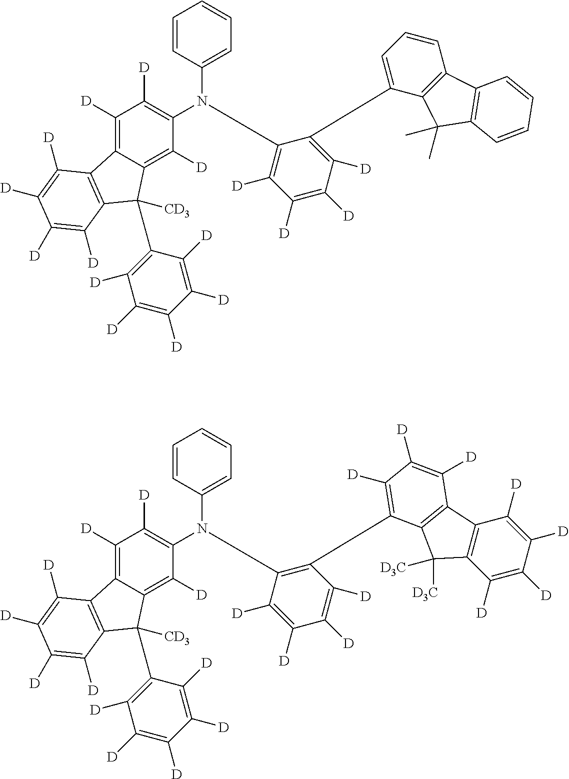 [SMILES: [2H]c1c([2H])c([2H])c(C2(C([2H])([2H])[2H])c3c([2H])c([2H])c([2H])c([2H])c3-c3c([2H])c([2H])c(N(c4ccccc4)c4c([2H])c([2H])c([2H])c([2H])c4-c4cccc5c4C(C)(C)c4ccccc4-5)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4ccccc4)c4c([2H])c([2H])c([2H])c([2H])c4-c4c([2H])c([2H])c([2H])c5c4C(C([2H])([2H])[2H])(C([2H])([2H])[2H])c4c([2H])c([2H])c([2H])c([2H])c4-5)c([2H])c32)c([2H])c1[2H]]

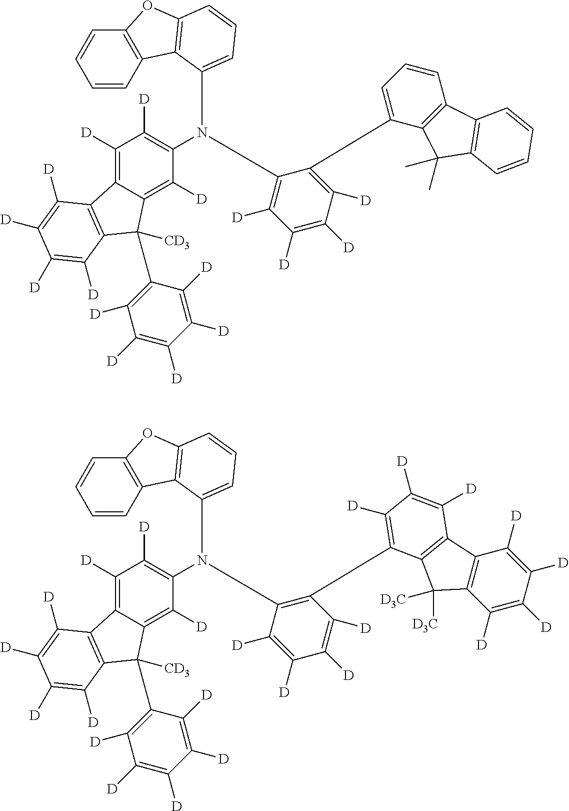 [SMILES: [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c([2H])c4-c4cccc5c4C(C)(C)c4ccccc4-5)c4cccc5oc6ccccc6c45)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c([2H])c4-c4c([2H])c([2H])c([2H])c5c4C(C([2H])([2H])[2H])(C([2H])([2H])[2H])c4c([2H])c([2H])c([2H])c([2H])c4-5)c4cccc5oc6ccccc6c45)c([2H])c32)c([2H])c1[2H]]

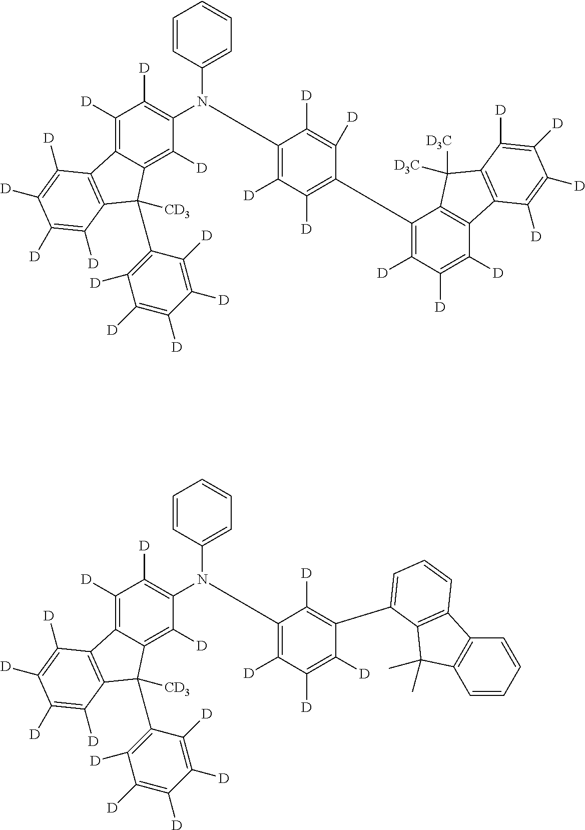 [SMILES: [2H]c1c([2H])c([2H])c(C2(C([2H])([2H])[2H])c3c([2H])c([2H])c([2H])c([2H])c3-c3c([2H])c([2H])c(N(c4ccccc4)c4c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c([2H])c4[2H])c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4ccccc4)c4c([2H])c([2H])c([2H])c(-c5cccc6c5C(C)(C)c5ccccc5-6)c4[2H])c([2H])c32)c([2H])c1[2H]]

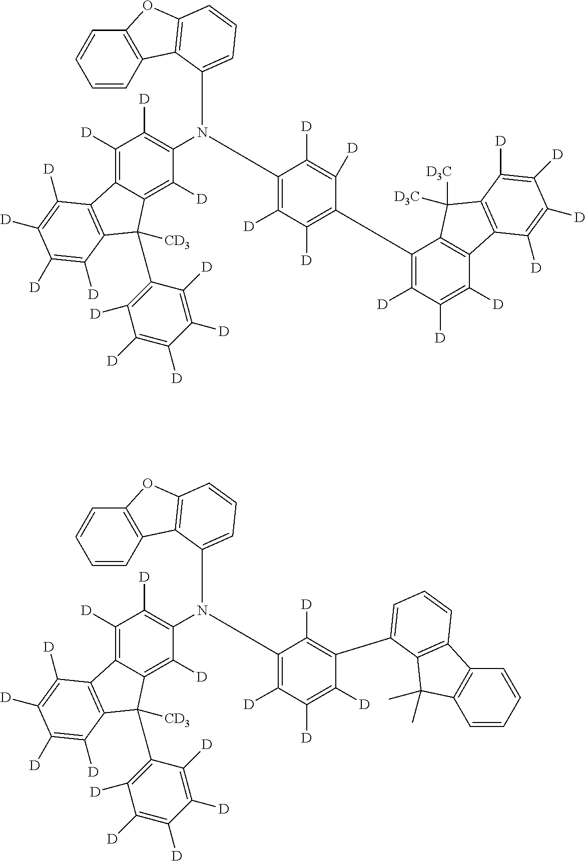 [SMILES: [2H]c1c([2H])c([2H])c(C2(C([2H])([2H])[2H])c3c([2H])c([2H])c([2H])c([2H])c3-c3c([2H])c([2H])c(N(c4c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c([2H])c4[2H])c4cccc5oc6ccccc6c45)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c(-c5cccc6c5C(C)(C)c5ccccc5-6)c4[2H])c4cccc5oc6ccccc6c45)c([2H])c32)c([2H])c1[2H]]

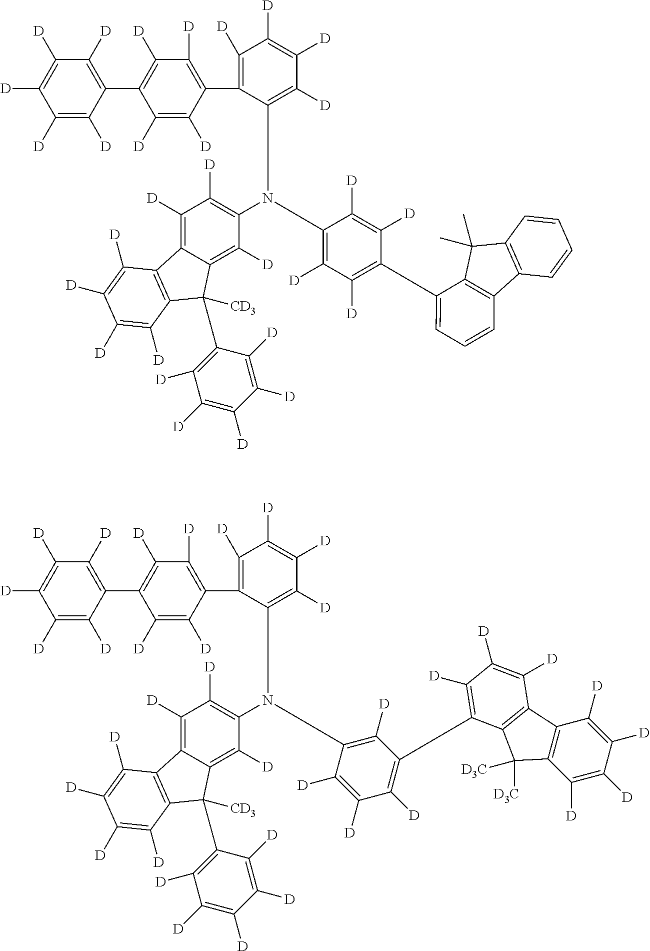

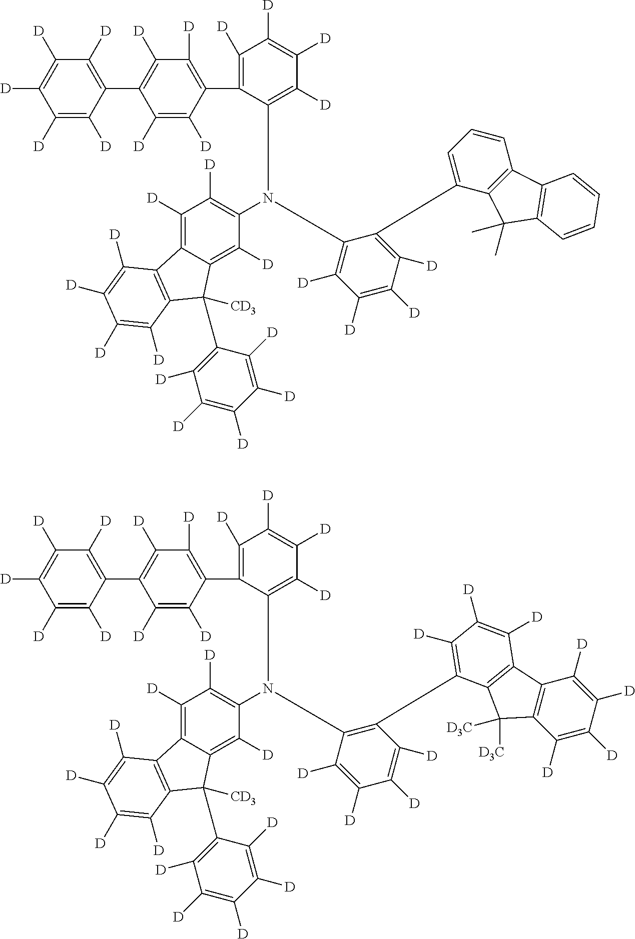

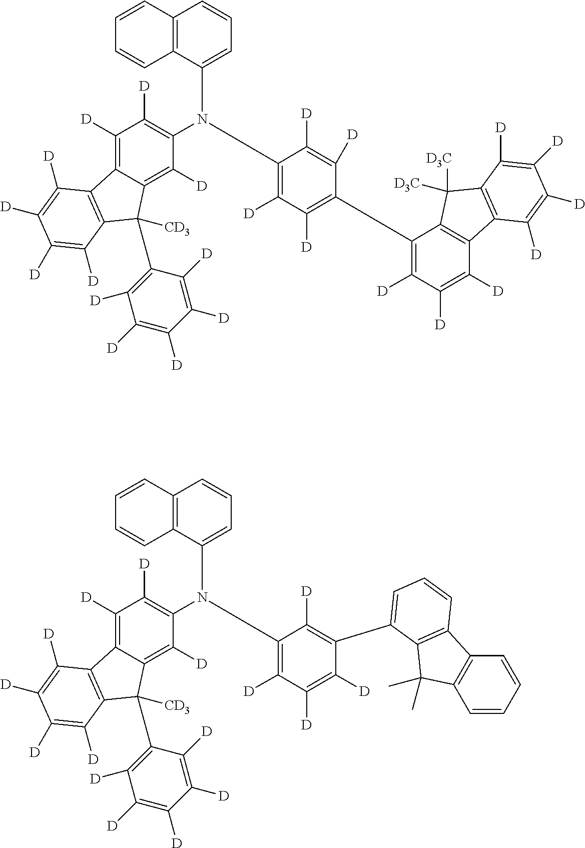 [SMILES: [2H]c1c([2H])c([2H])c(C2(C([2H])([2H])[2H])c3c([2H])c([2H])c([2H])c([2H])c3-c3c([2H])c([2H])c(N(c4c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c([2H])c4[2H])c4cccc5ccccc45)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c(-c5cccc6c5C(C)(C)c5ccccc5-6)c4[2H])c4cccc5ccccc45)c([2H])c32)c([2H])c1[2H]]

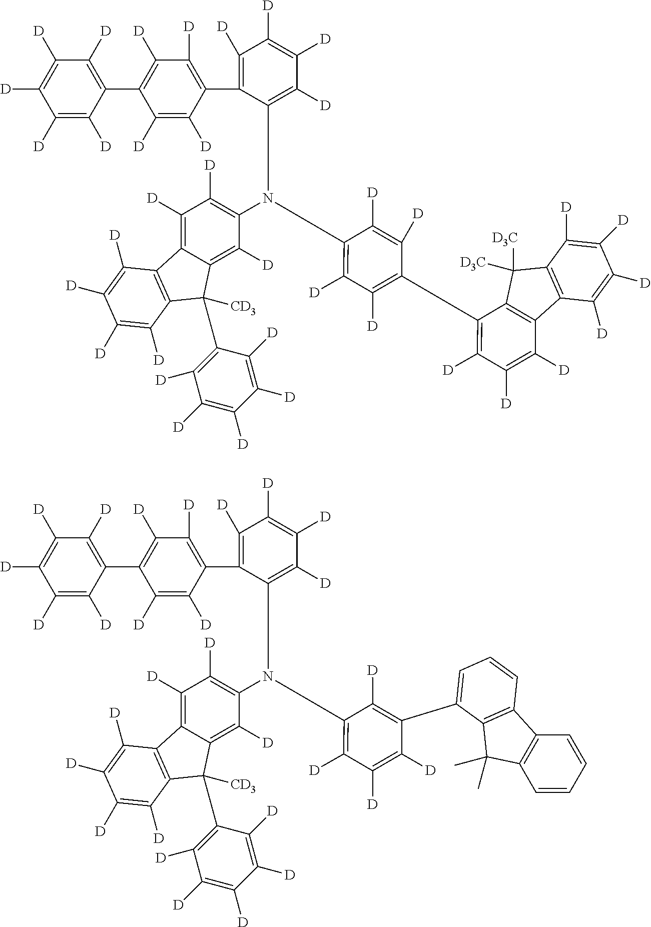

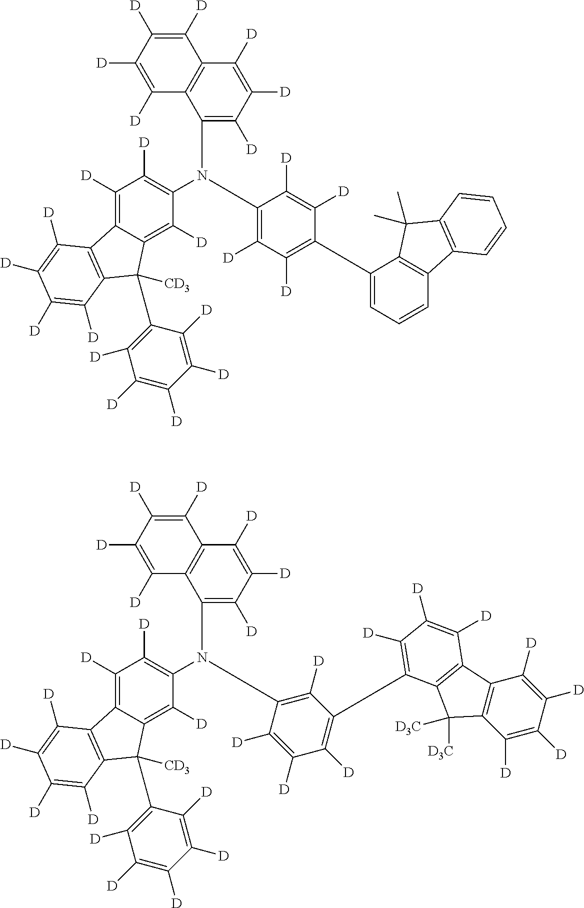

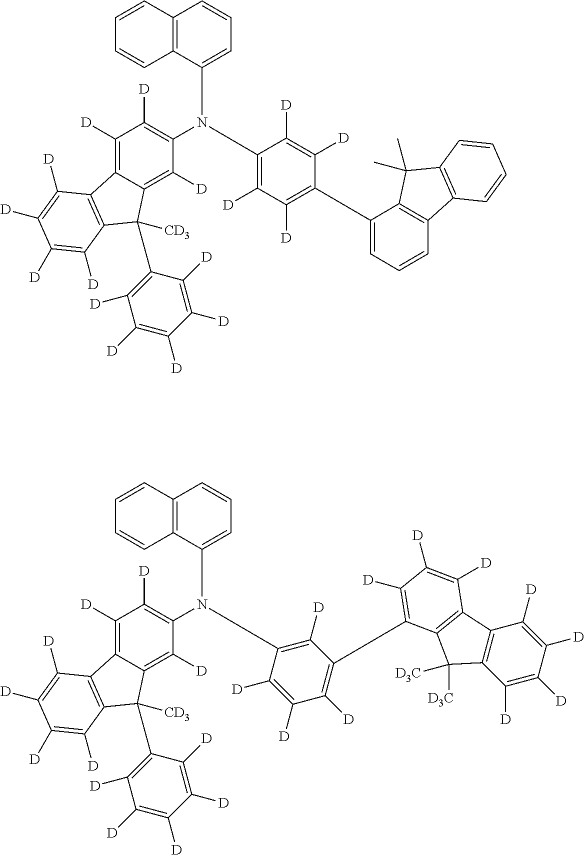 [SMILES: [2H]c1c([2H])c([2H])c(C2(C([2H])([2H])[2H])c3c([2H])c([2H])c([2H])c([2H])c3-c3c([2H])c([2H])c(N(c4c([2H])c([2H])c(-c5cccc6c5C(C)(C)c5ccccc5-6)c([2H])c4[2H])c4cccc5ccccc45)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c(-c5c([2H])c([2H])c([2H])c6c5C(C([2H])([2H])[2H])(C([2H])([2H])[2H])c5c([2H])c([2H])c([2H])c([2H])c5-6)c4[2H])c4cccc5ccccc45)c([2H])c32)c([2H])c1[2H]]

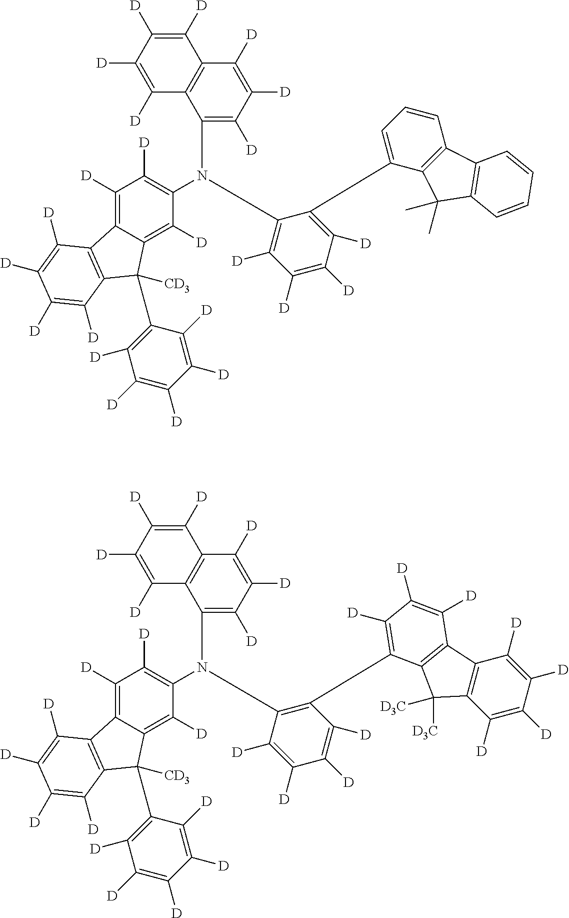

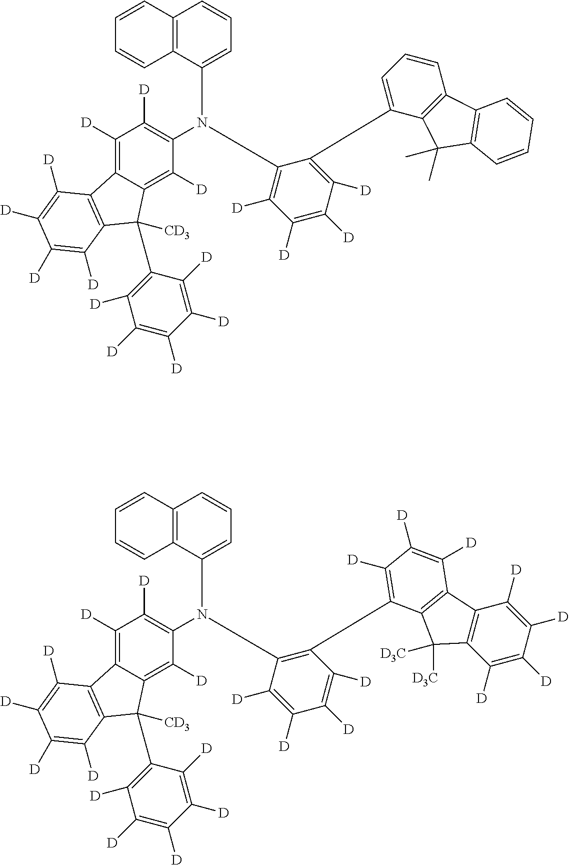 [SMILES: [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c([2H])c4-c4cccc5c4C(C)(C)c4ccccc4-5)c4cccc5ccccc45)c([2H])c32)c([2H])c1[2H] ; [2H]c1c([2H])c([2H])c(C2(C([2H])([2H])[2H])c3c([2H])c([2H])c([2H])c([2H])c3-c3c([2H])c([2H])c(N(c4c([2H])c([2H])c([2H])c([2H])c4-c4c([2H])c([2H])c([2H])c5c4C(C([2H])([2H])[2H])(C([2H])([2H])[2H])c4c([2H])c([2H])c([2H])c([2H])c4-5)c4cccc5ccccc45)c([2H])c32)c([2H])c1[2H]]

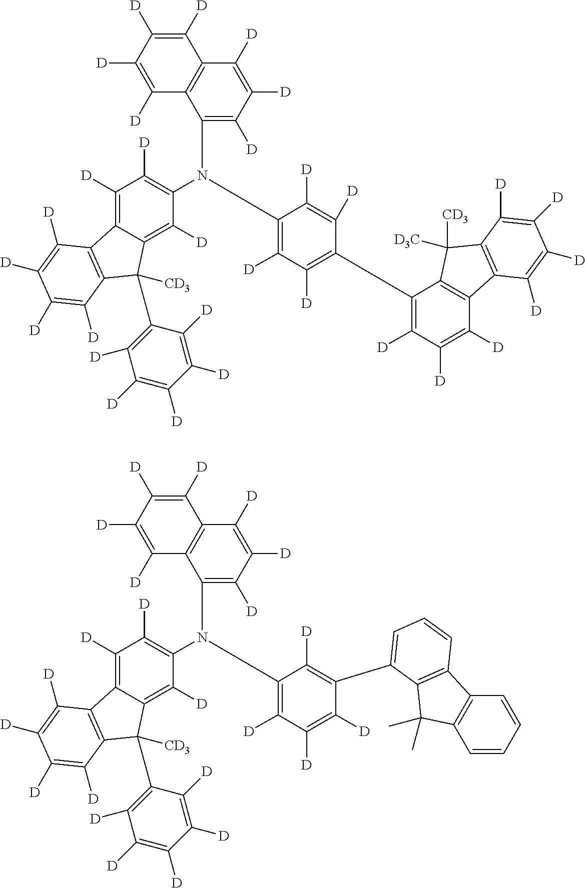

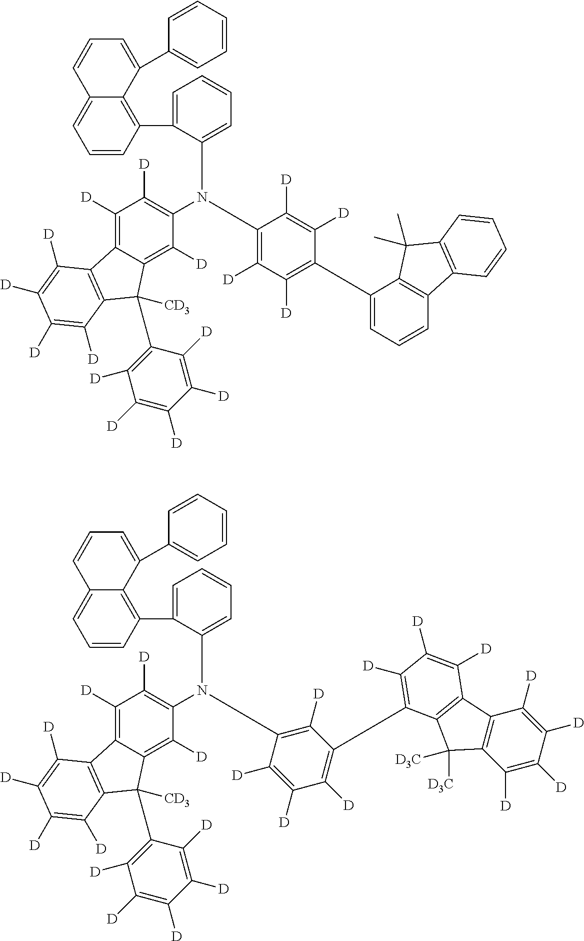

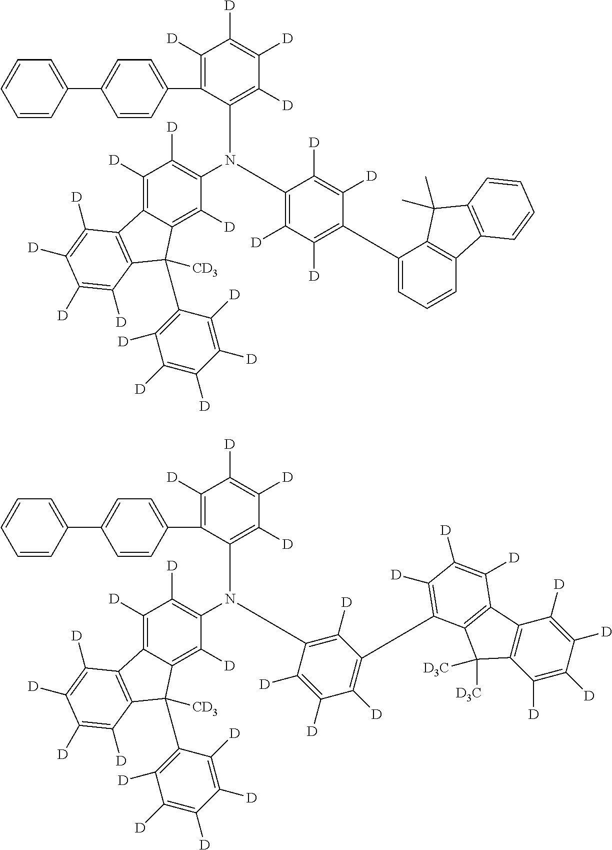

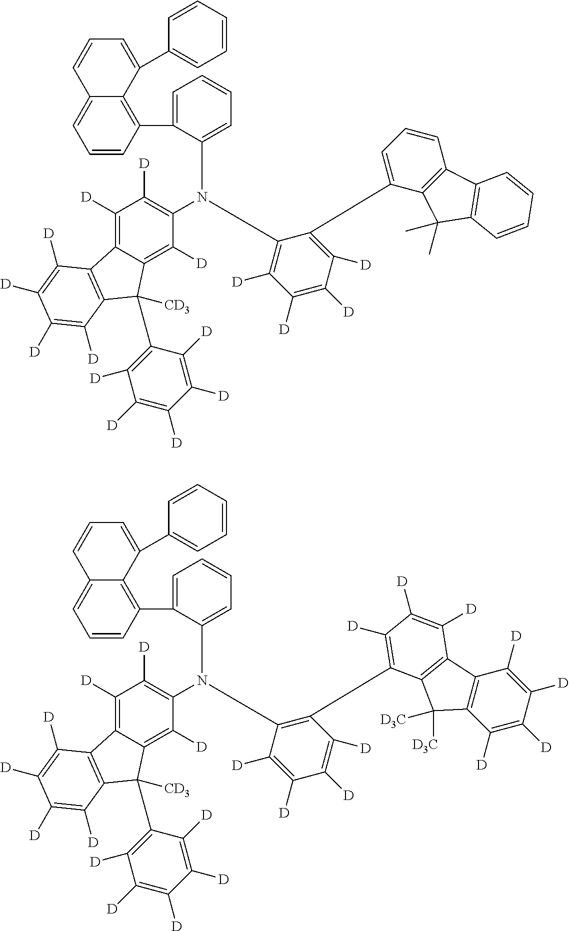

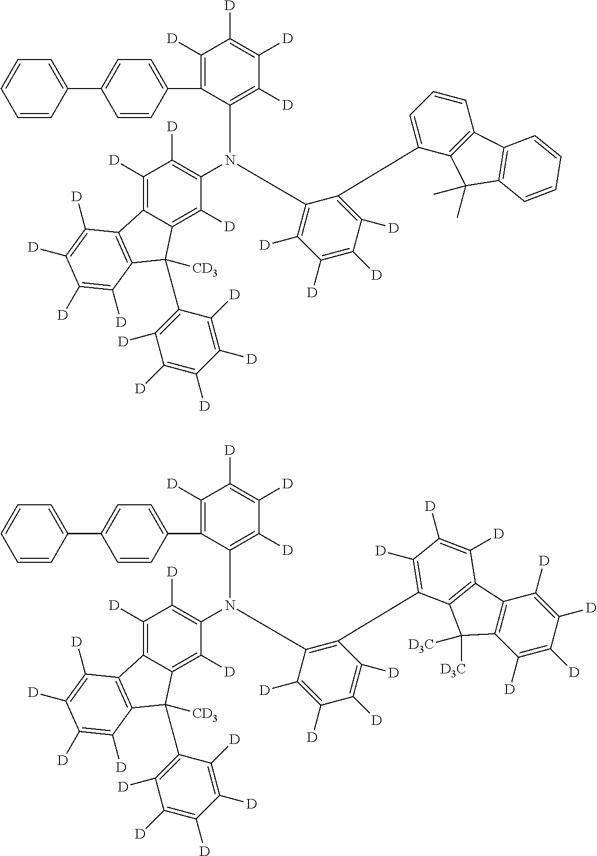

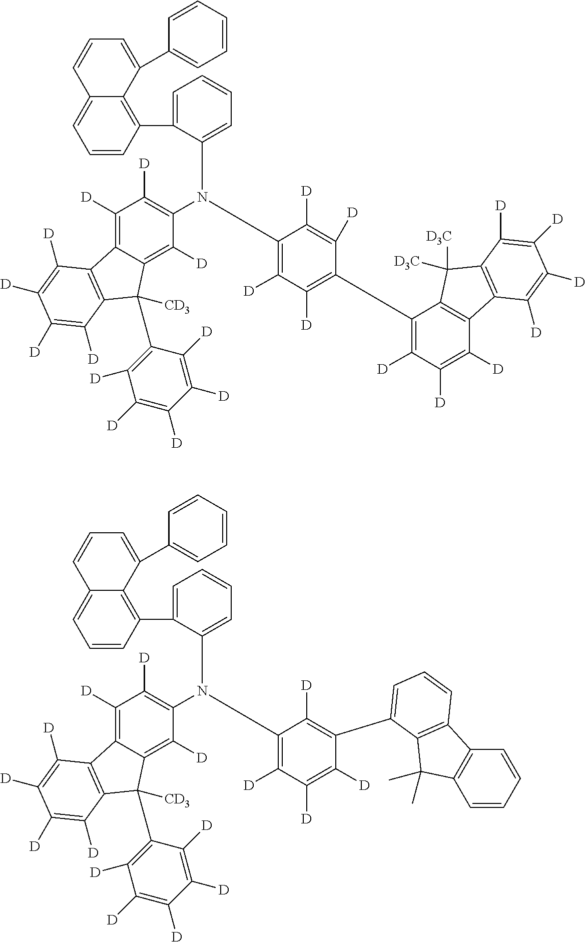

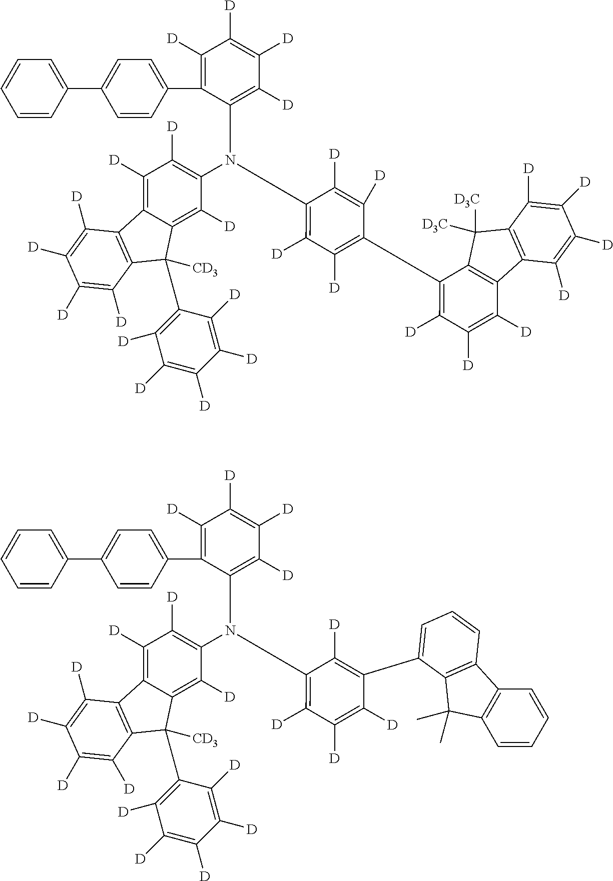

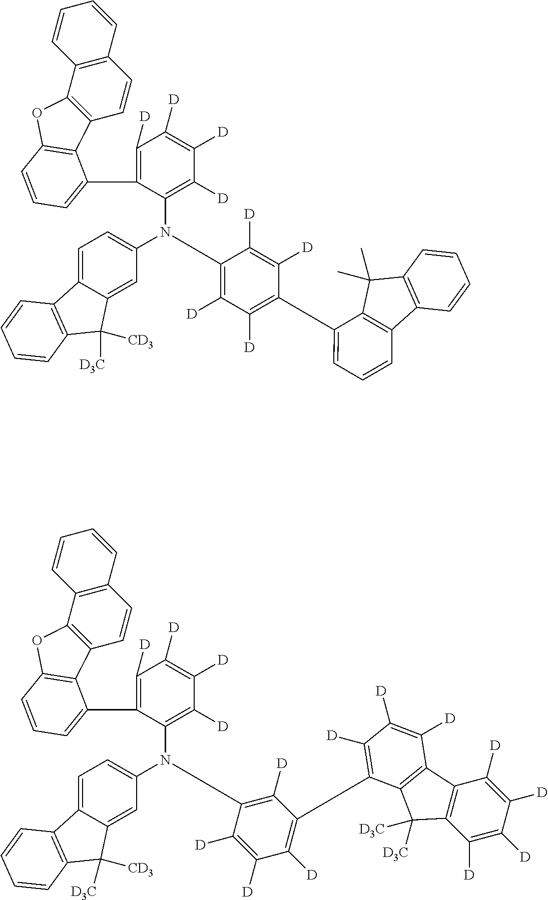 [SMILES: [2H]c1c([2H])c([2H])c(N(c2ccc3c(c2)C(C([2H])([2H])[2H])(C([2H])([2H])[2H])c2ccccc2-3)c2c([2H])c([2H])c(-c3cccc4c3C(C)(C)c3ccccc3-4)c([2H])c2[2H])c(-c2cccc3oc4c5ccccc5ccc4c23)c1[2H] ; [2H]c1c([2H])c([2H])c(N(c2ccc3c(c2)C(C([2H])([2H])[2H])(C([2H])([2H])[2H])c2ccccc2-3)c2c([2H])c([2H])c([2H])c(-c3c([2H])c([2H])c([2H])c4c3C(C([2H])([2H])[2H])(C([2H])([2H])[2H])c3c([2H])c([2H])c([2H])c([2H])c3-4)c2[2H])c(-c2cccc3oc4c5ccccc5ccc4c23)c1[2H]]

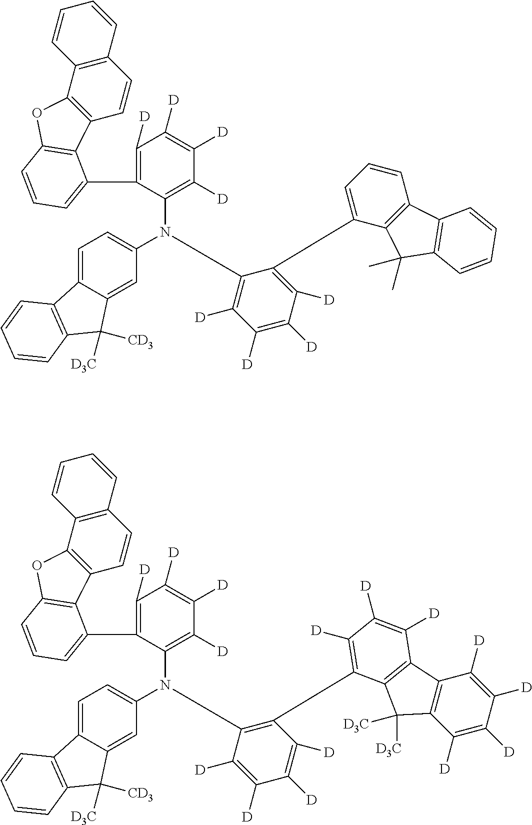 [SMILES: [2H]c1c([2H])c([2H])c(N(c2ccc3c(c2)C(C([2H])([2H])[2H])(C([2H])([2H])[2H])c2ccccc2-3)c2c([2H])c([2H])c([2H])c([2H])c2-c2cccc3oc4c5ccccc5ccc4c23)c(-c2cccc3c2C(C)(C)c2ccccc2-3)c1[2H] ; [2H]c1c([2H])c([2H])c(N(c2ccc3c(c2)C(C([2H])([2H])[2H])(C([2H])([2H])[2H])c2ccccc2-3)c2c([2H])c([2H])c([2H])c([2H])c2-c2cccc3oc4c5ccccc5ccc4c23)c(-c2c([2H])c([2H])c([2H])c3c2C(C([2H])([2H])[2H])(C([2H])([2H])[2H])c2c([2H])c([2H])c([2H])c([2H])c2-3)c1[2H]]

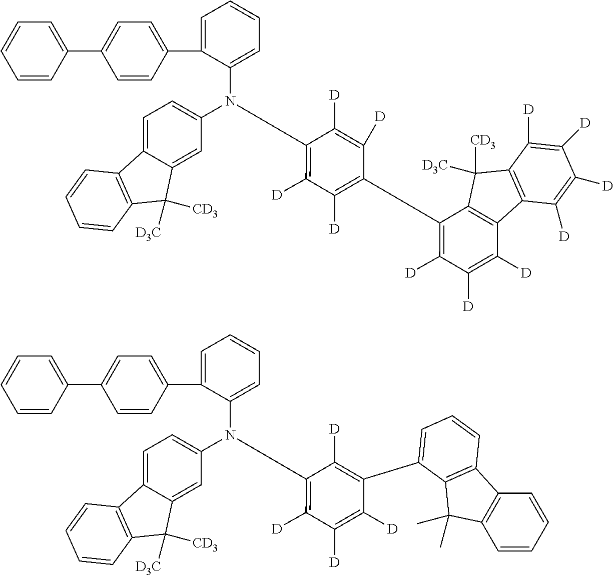 [SMILES: [2H]c1c([2H])c([2H])c2c(c1[2H])-c1c([2H])c([2H])c([2H])c(-c3c([2H])c([2H])c(N(c4ccc5c(c4)C(C([2H])([2H])[2H])(C([2H])([2H])[2H])c4ccccc4-5)c4ccccc4-c4ccc(-c5ccccc5)cc4)c([2H])c3[2H])c1C2(C([2H])([2H])[2H])C([2H])([2H])[2H] ; [2H]c1c([2H])c(-c2cccc3c2C(C)(C)c2ccccc2-3)c([2H])c(N(c2ccc3c(c2)C(C([2H])([2H])[2H])(C([2H])([2H])[2H])c2ccccc2-3)c2ccccc2-c2ccc(-c3ccccc3)cc2)c1[2H]]

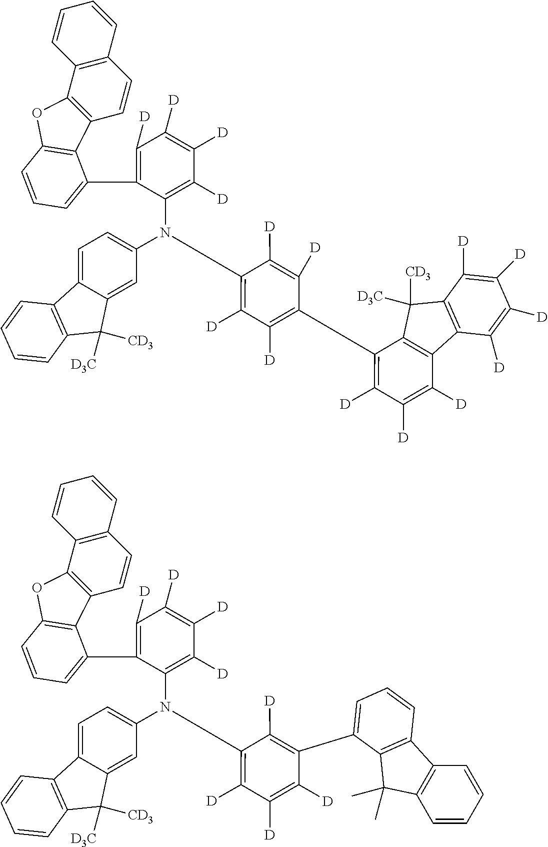 [SMILES: [2H]c1c([2H])c([2H])c(N(c2ccc3c(c2)C(C([2H])([2H])[2H])(C([2H])([2H])[2H])c2ccccc2-3)c2c([2H])c([2H])c(-c3c([2H])c([2H])c([2H])c4c3C(C([2H])([2H])[2H])(C([2H])([2H])[2H])c3c([2H])c([2H])c([2H])c([2H])c3-4)c([2H])c2[2H])c(-c2cccc3oc4c5ccccc5ccc4c23)c1[2H] ; [2H]c1c([2H])c([2H])c(N(c2ccc3c(c2)C(C([2H])([2H])[2H])(C([2H])([2H])[2H])c2ccccc2-3)c2c([2H])c([2H])c([2H])c(-c3cccc4c3C(C)(C)c3ccccc3-4)c2[2H])c(-c2cccc3oc4c5ccccc5ccc4c23)c1[2H]]

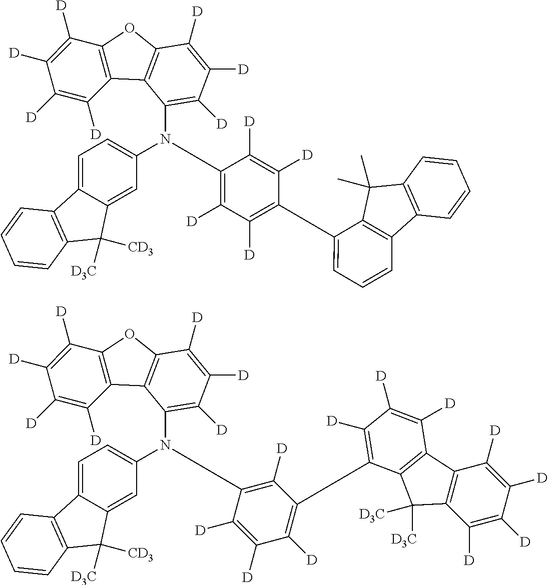

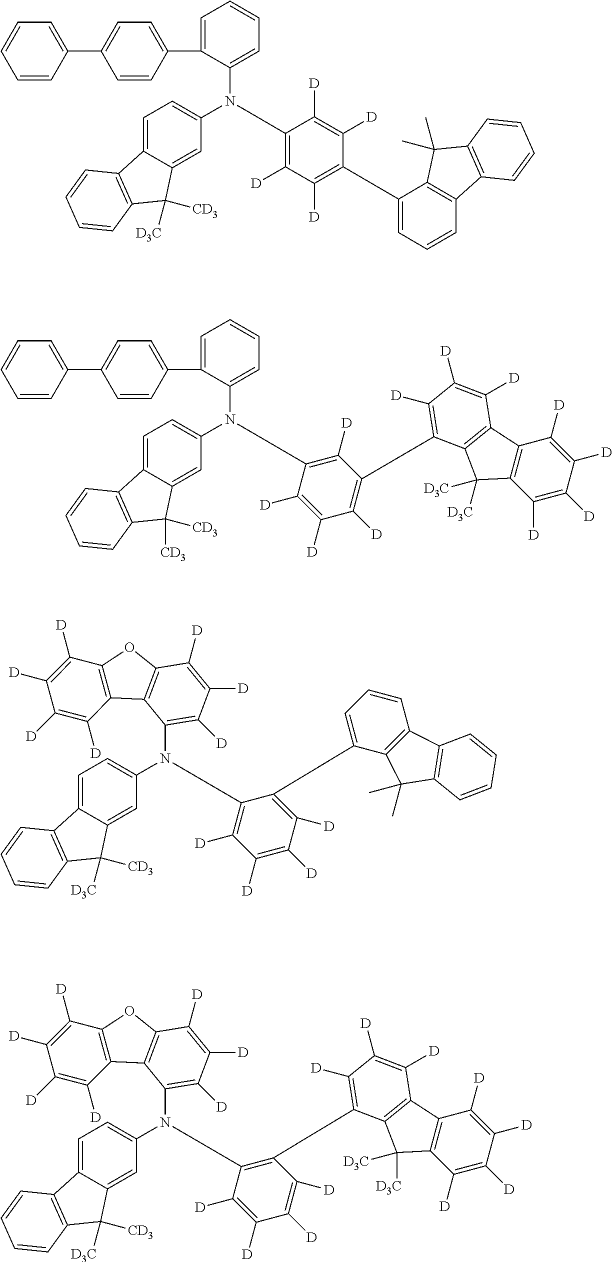

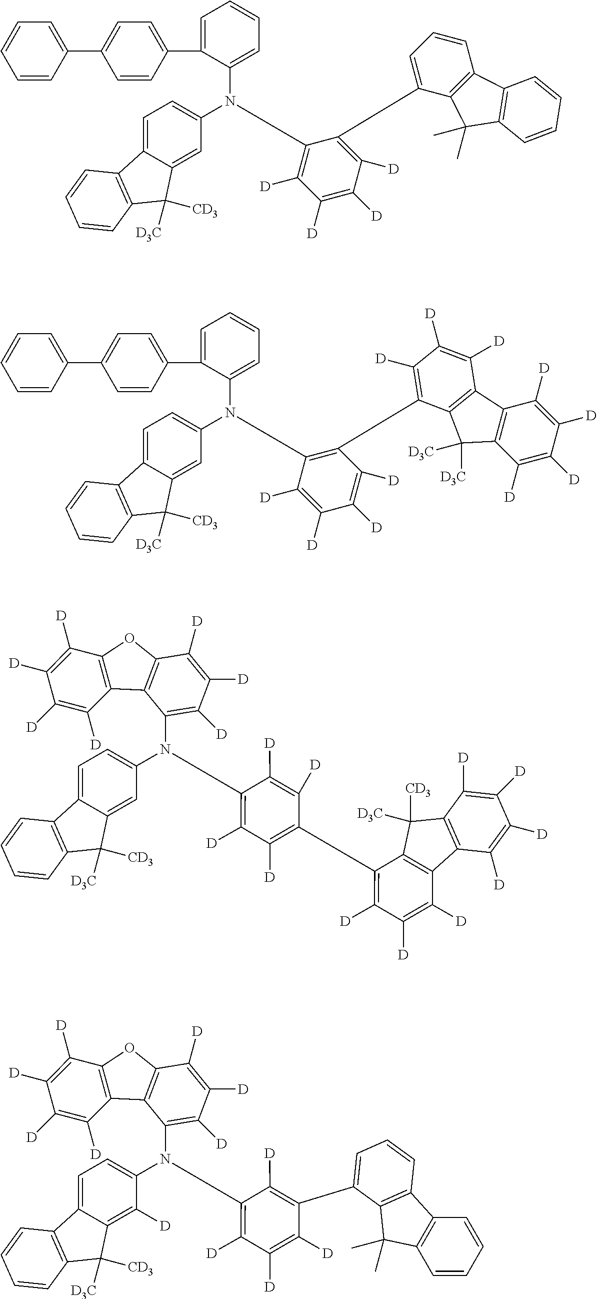

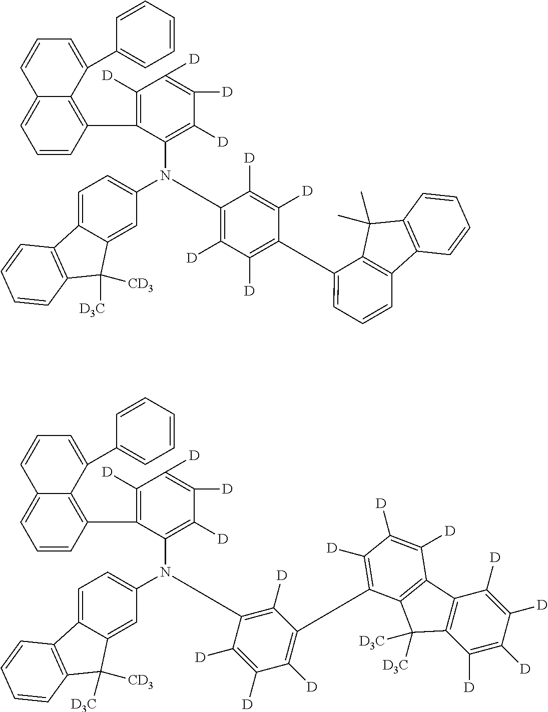 [SMILES: [2H]c1c([2H])c([2H])c(N(c2ccc3c(c2)C(C([2H])([2H])[2H])(C([2H])([2H])[2H])c2ccccc2-3)c2c([2H])c([2H])c(-c3cccc4c3C(C)(C)c3ccccc3-4)c([2H])c2[2H])c(-c2cccc3cccc(-c4ccccc4)c23)c1[2H] ; [2H]c1c([2H])c([2H])c(N(c2ccc3c(c2)C(C([2H])([2H])[2H])(C([2H])([2H])[2H])c2ccccc2-3)c2c([2H])c([2H])c([2H])c(-c3c([2H])c([2H])c([2H])c4c3C(C([2H])([2H])[2H])(C([2H])([2H])[2H])c3c([2H])c([2H])c([2H])c([2H])c3-4)c2[2H])c(-c2cccc3cccc(-c4ccccc4)c23)c1[2H]]

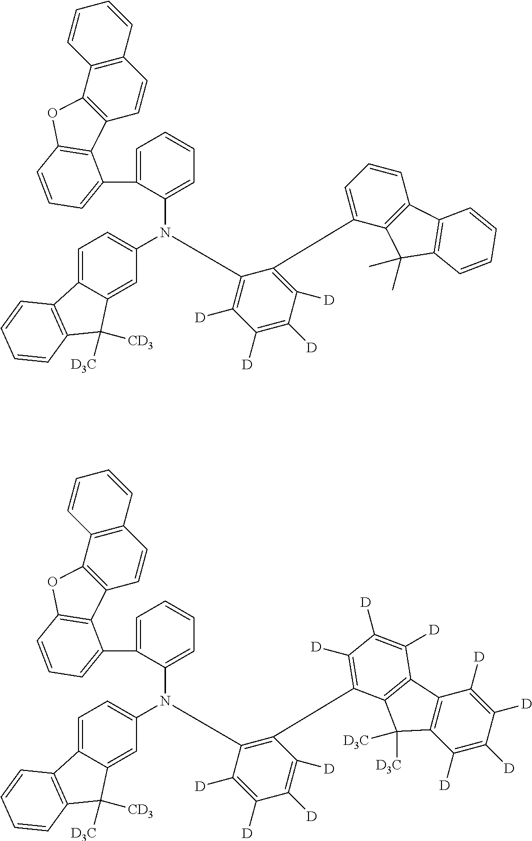 [SMILES: [2H]c1c([2H])c([2H])c(N(c2ccc3c(c2)C(C([2H])([2H])[2H])(C([2H])([2H])[2H])c2ccccc2-3)c2ccccc2-c2cccc3oc4c5ccccc5ccc4c23)c(-c2cccc3c2C(C)(C)c2ccccc2-3)c1[2H] ; [2H]c1c([2H])c([2H])c(N(c2ccc3c(c2)C(C([2H])([2H])[2H])(C([2H])([2H])[2H])c2ccccc2-3)c2ccccc2-c2cccc3oc4c5ccccc5ccc4c23)c(-c2c([2H])c([2H])c([2H])c3c2C(C([2H])([2H])[2H])(C([2H])([2H])[2H])c2c([2H])c([2H])c([2H])c([2H])c2-3)c1[2H]]

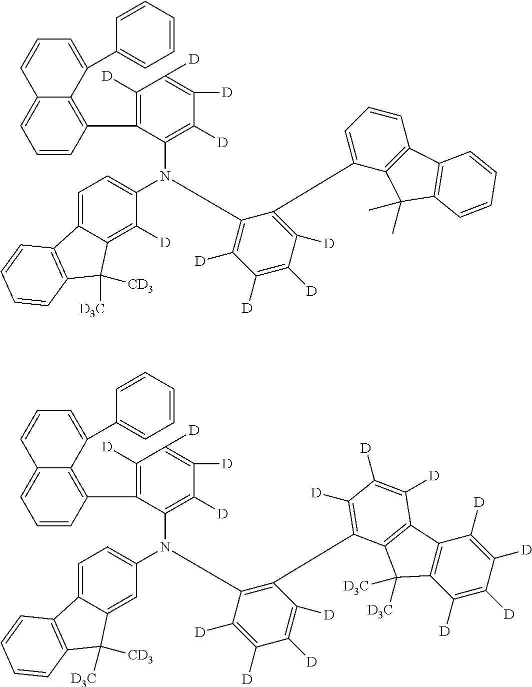 [SMILES: [2H]c1c([2H])c([2H])c(N(c2ccc3c(c2[2H])C(C([2H])([2H])[2H])(C([2H])([2H])[2H])c2ccccc2-3)c2c([2H])c([2H])c([2H])c([2H])c2-c2cccc3cccc(-c4ccccc4)c23)c(-c2cccc3c2C(C)(C)c2ccccc2-3)c1[2H] ; [2H]c1c([2H])c([2H])c(N(c2ccc3c(c2)C(C([2H])([2H])[2H])(C([2H])([2H])[2H])c2ccccc2-3)c2c([2H])c([2H])c([2H])c([2H])c2-c2cccc3cccc(-c4ccccc4)c23)c(-c2c([2H])c([2H])c([2H])c3c2C(C([2H])([2H])[2H])(C([2H])([2H])[2H])c2c([2H])c([2H])c([2H])c([2H])c2-3)c1[2H]]

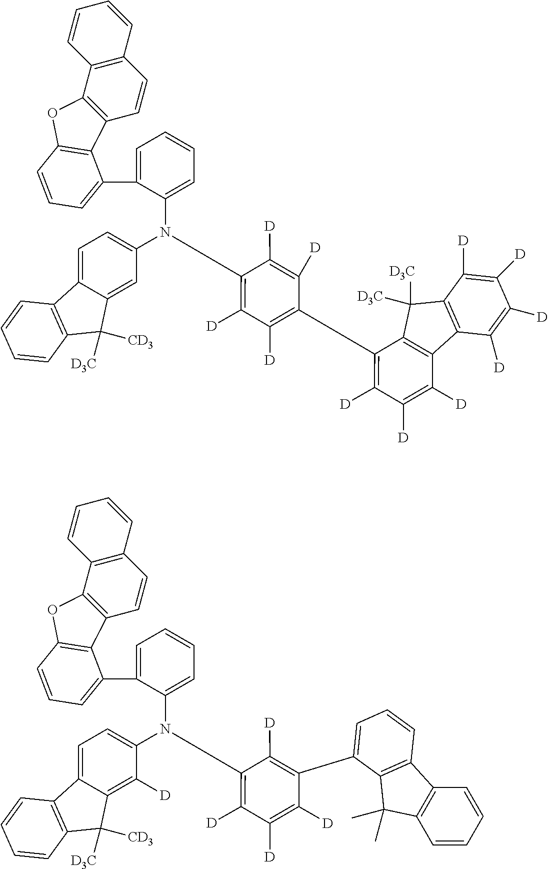 [SMILES: [2H]c1c([2H])c([2H])c2c(c1[2H])-c1c([2H])c([2H])c([2H])c(-c3c([2H])c([2H])c(N(c4ccc5c(c4)C(C([2H])([2H])[2H])(C([2H])([2H])[2H])c4ccccc4-5)c4ccccc4-c4cccc5oc6c7ccccc7ccc6c45)c([2H])c3[2H])c1C2(C([2H])([2H])[2H])C([2H])([2H])[2H] ; [2H]c1c([2H])c(-c2cccc3c2C(C)(C)c2ccccc2-3)c([2H])c(N(c2ccccc2-c2cccc3oc4c5ccccc5ccc4c23)c2ccc3c(c2[2H])C(C([2H])([2H])[2H])(C([2H])([2H])[2H])c2ccccc2-3)c1[2H]]

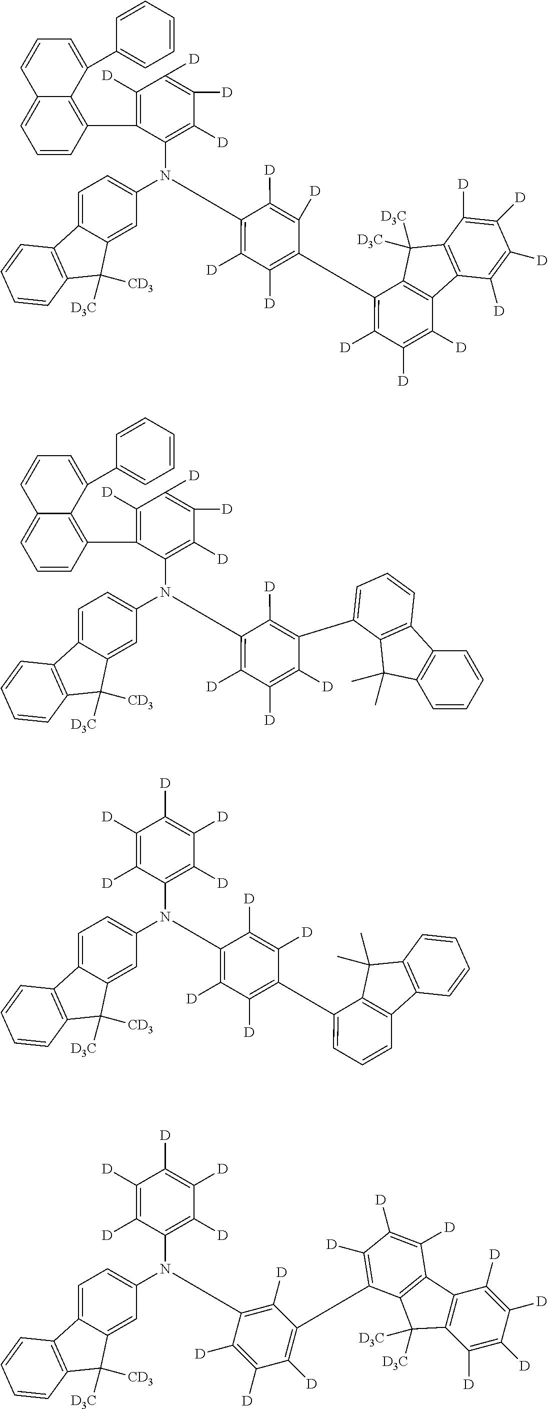

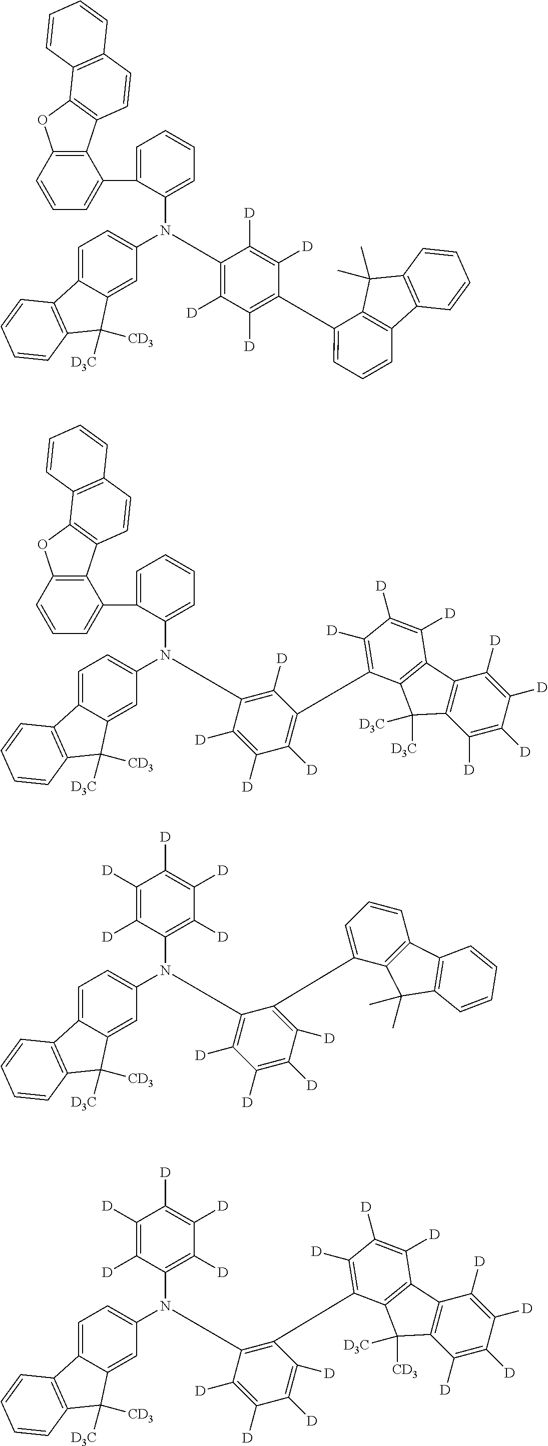

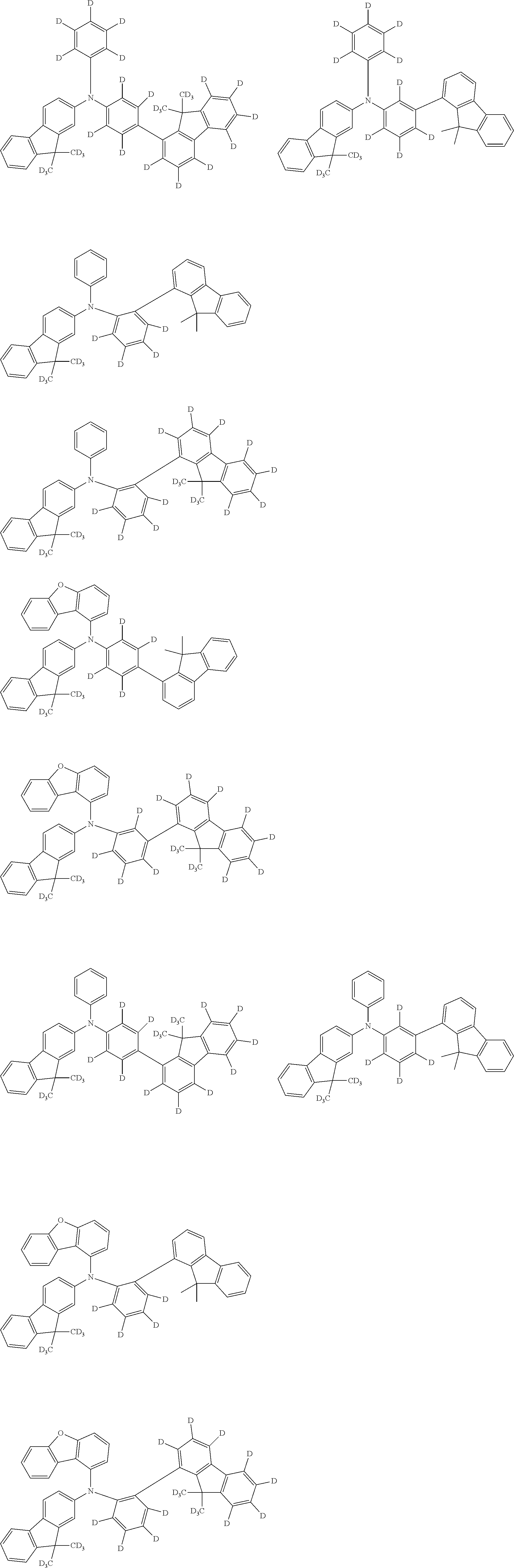

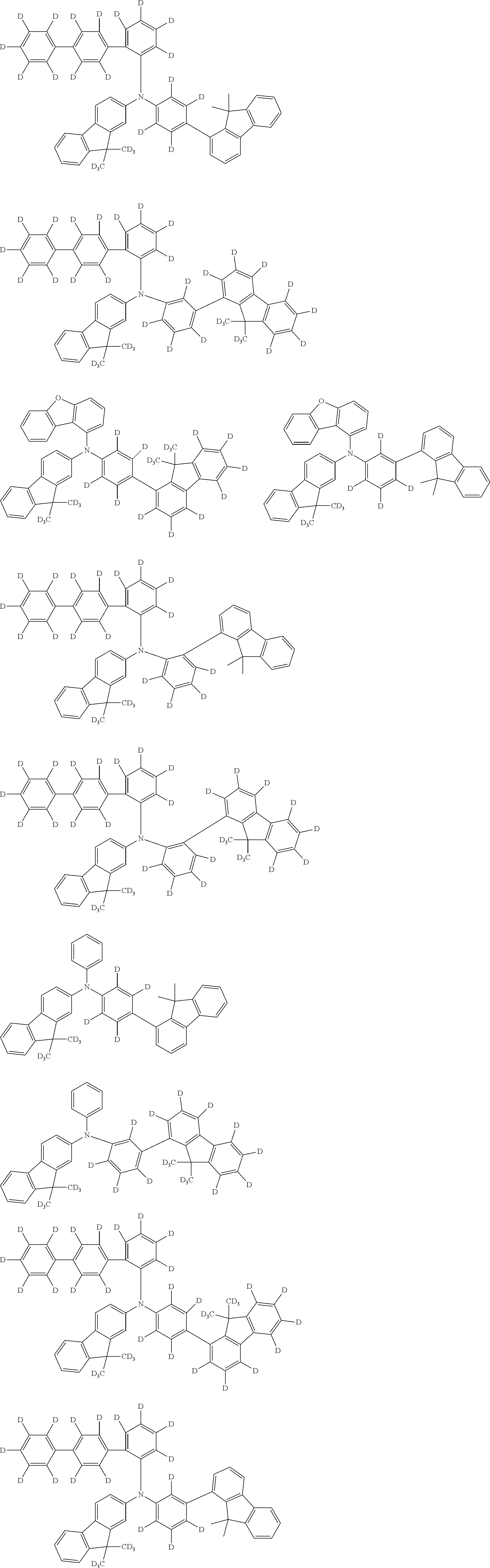

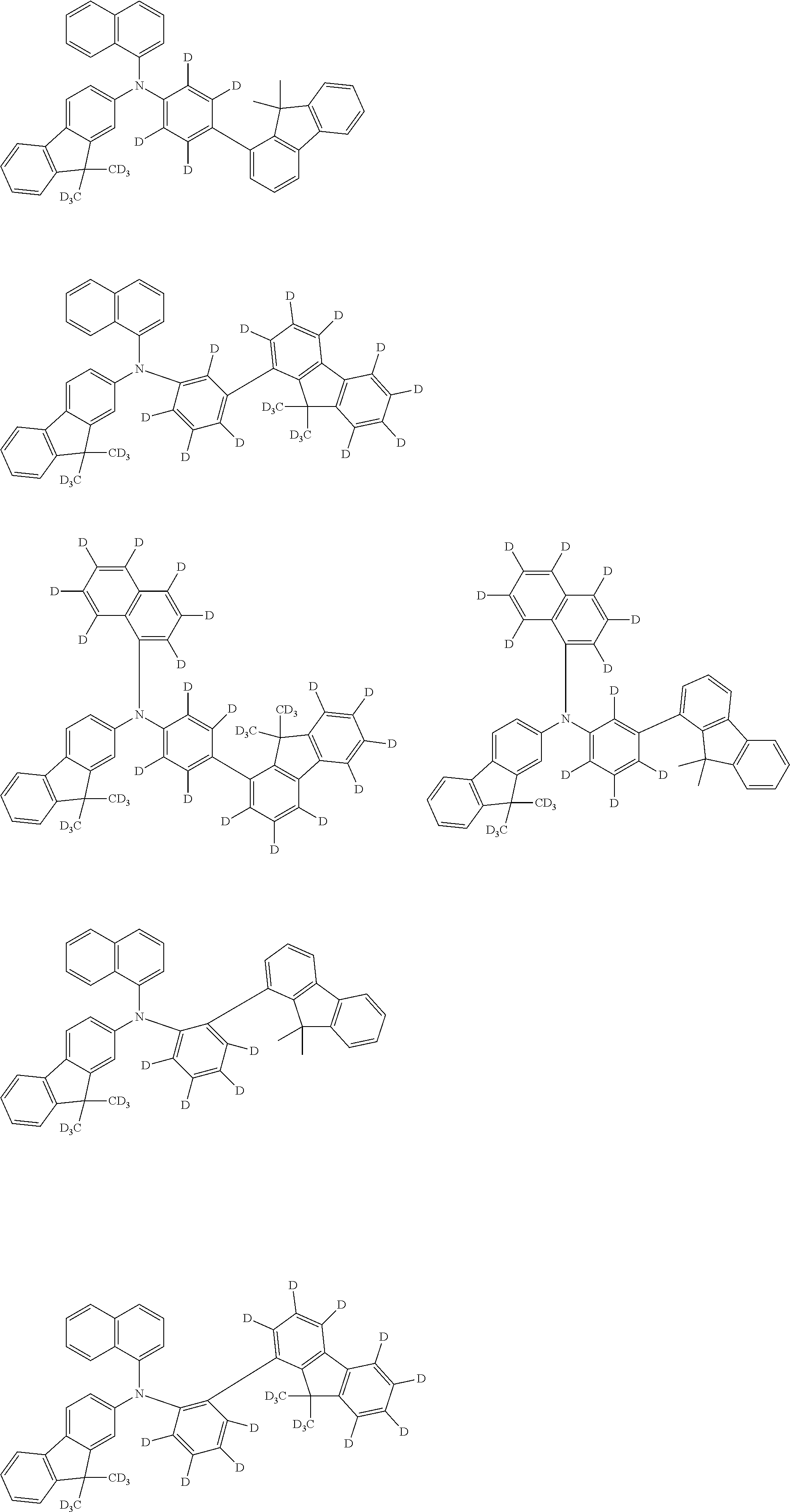

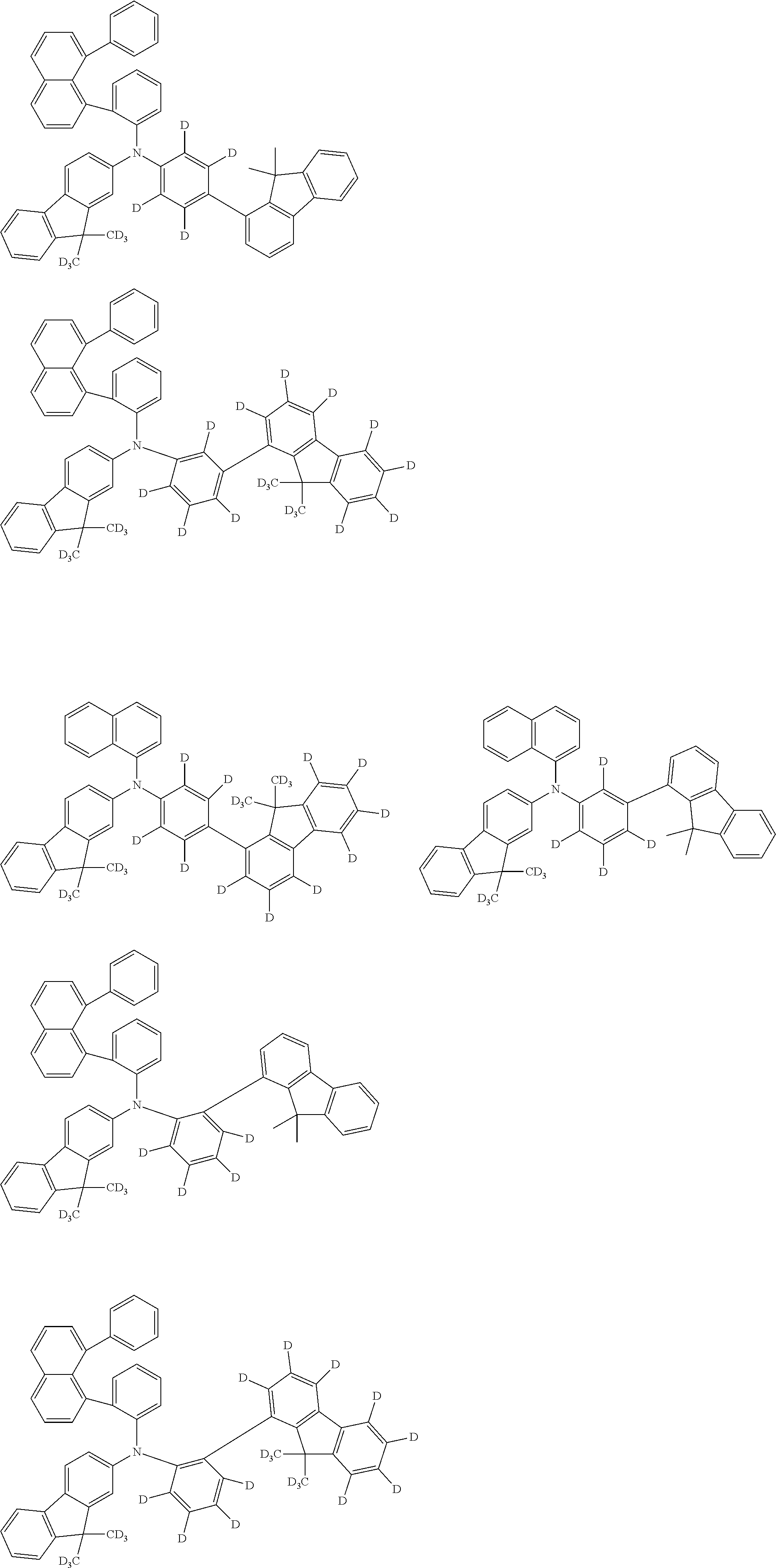

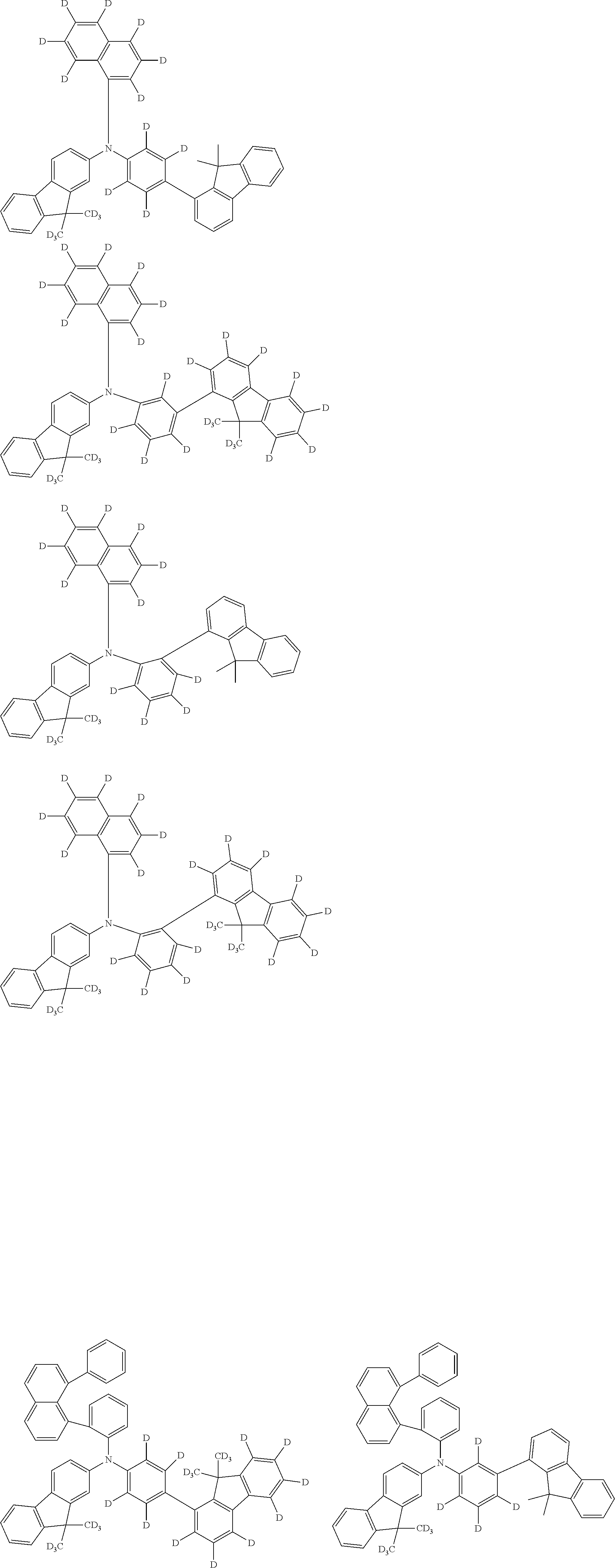

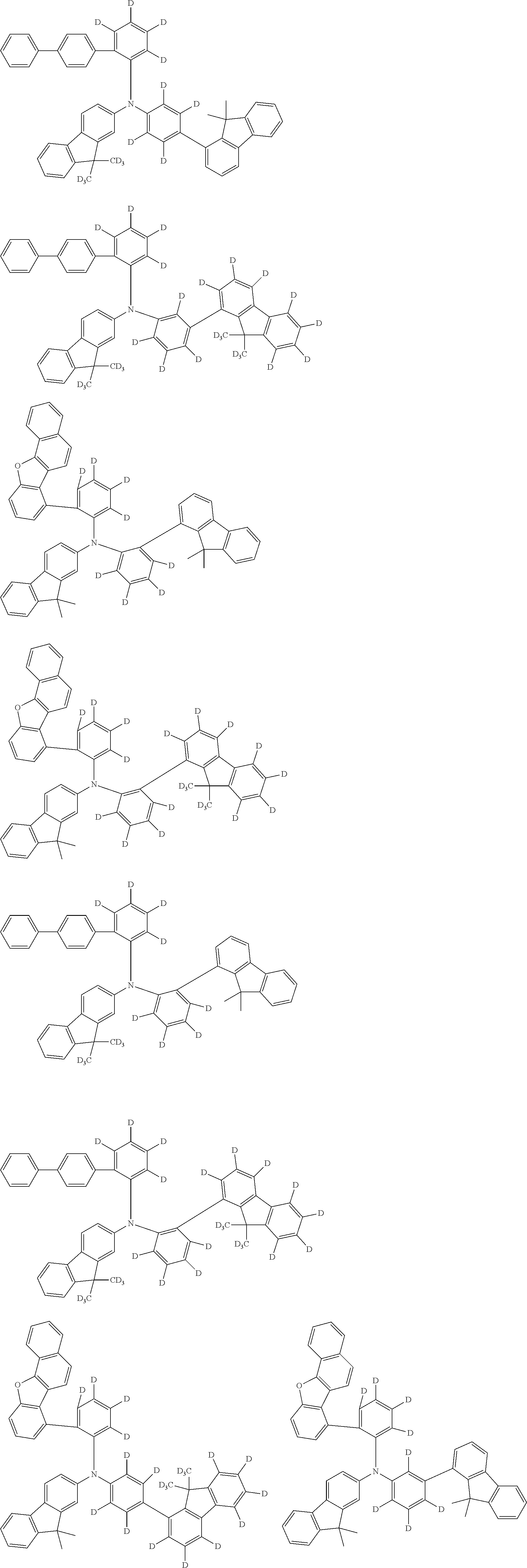

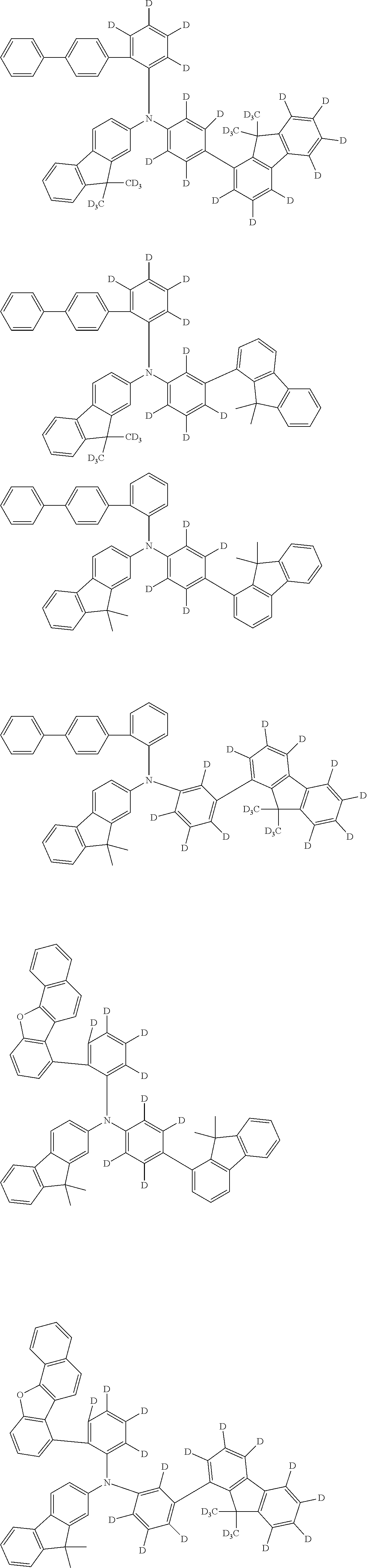

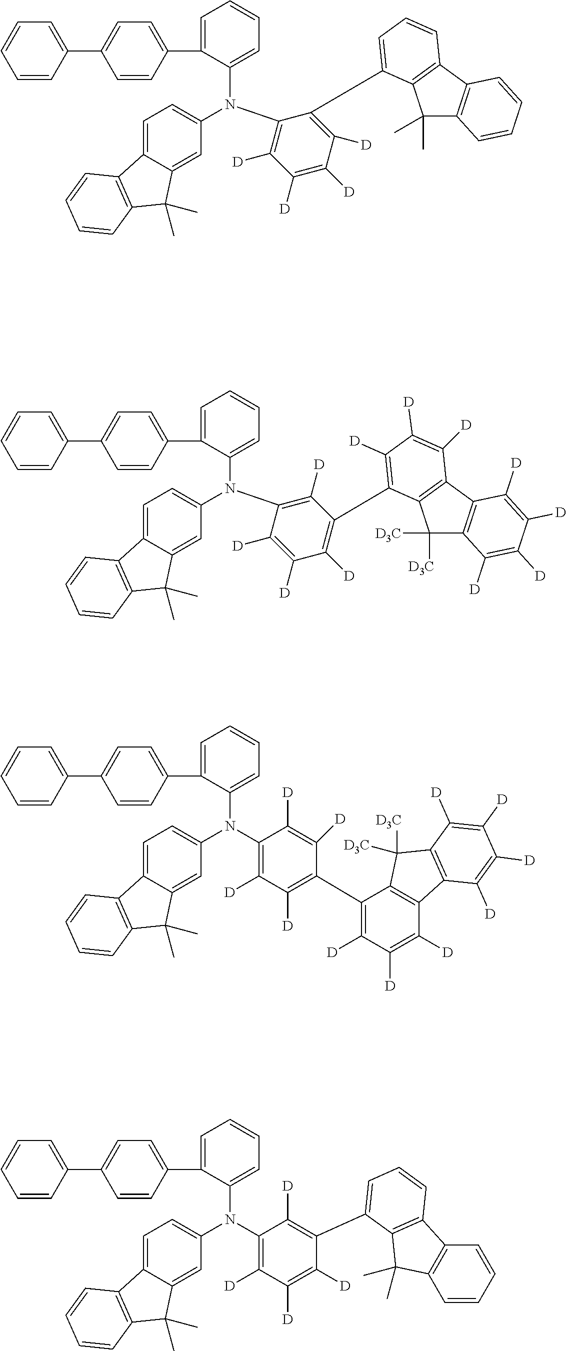

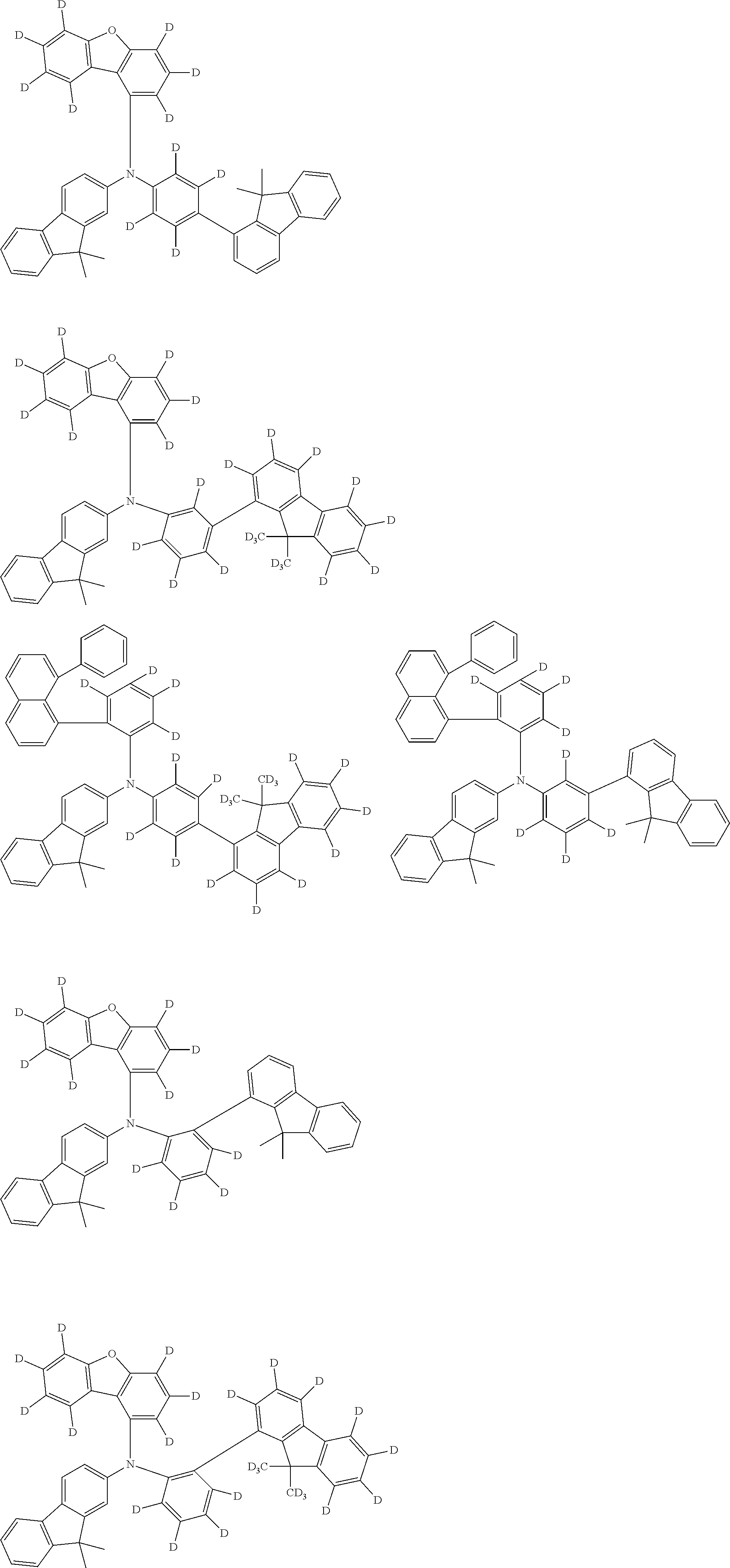

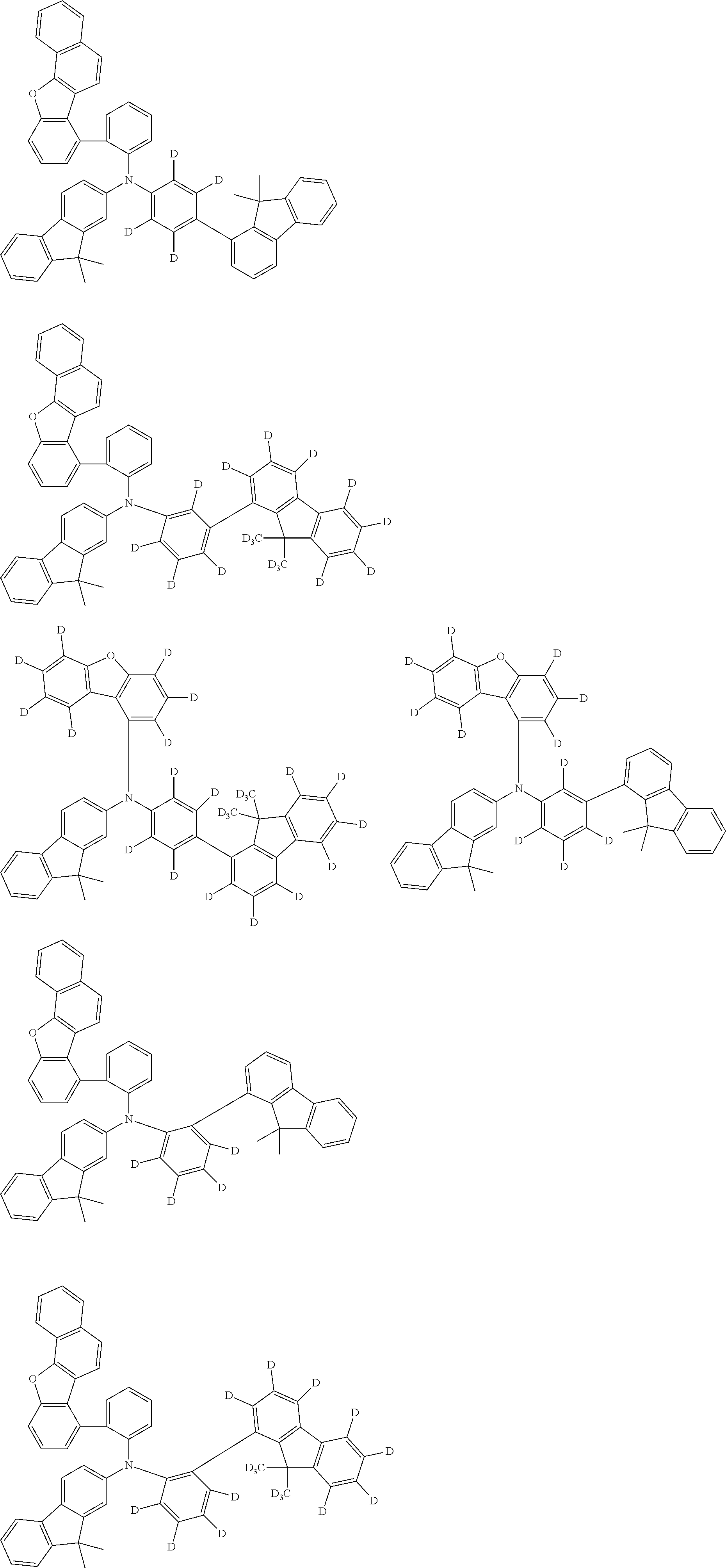

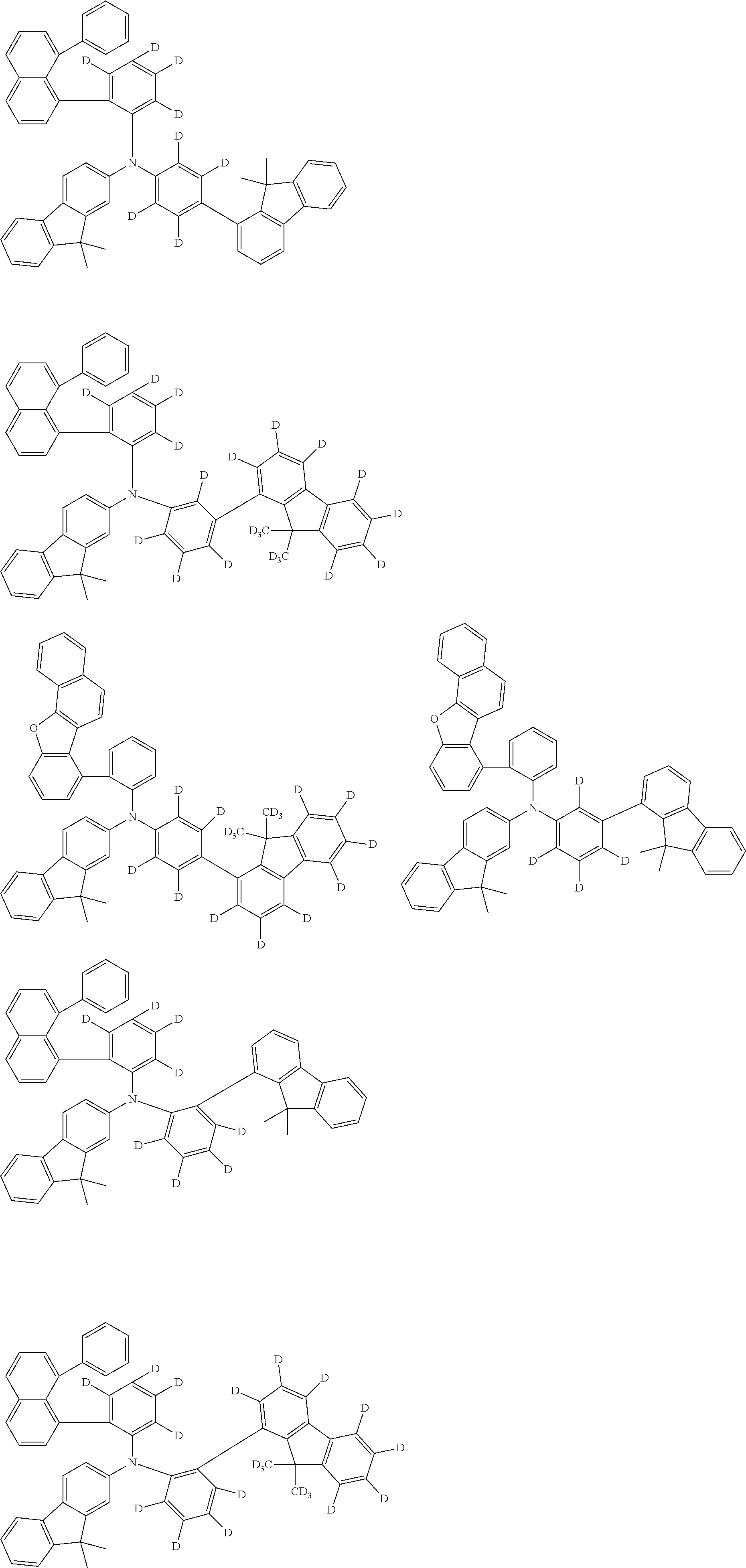

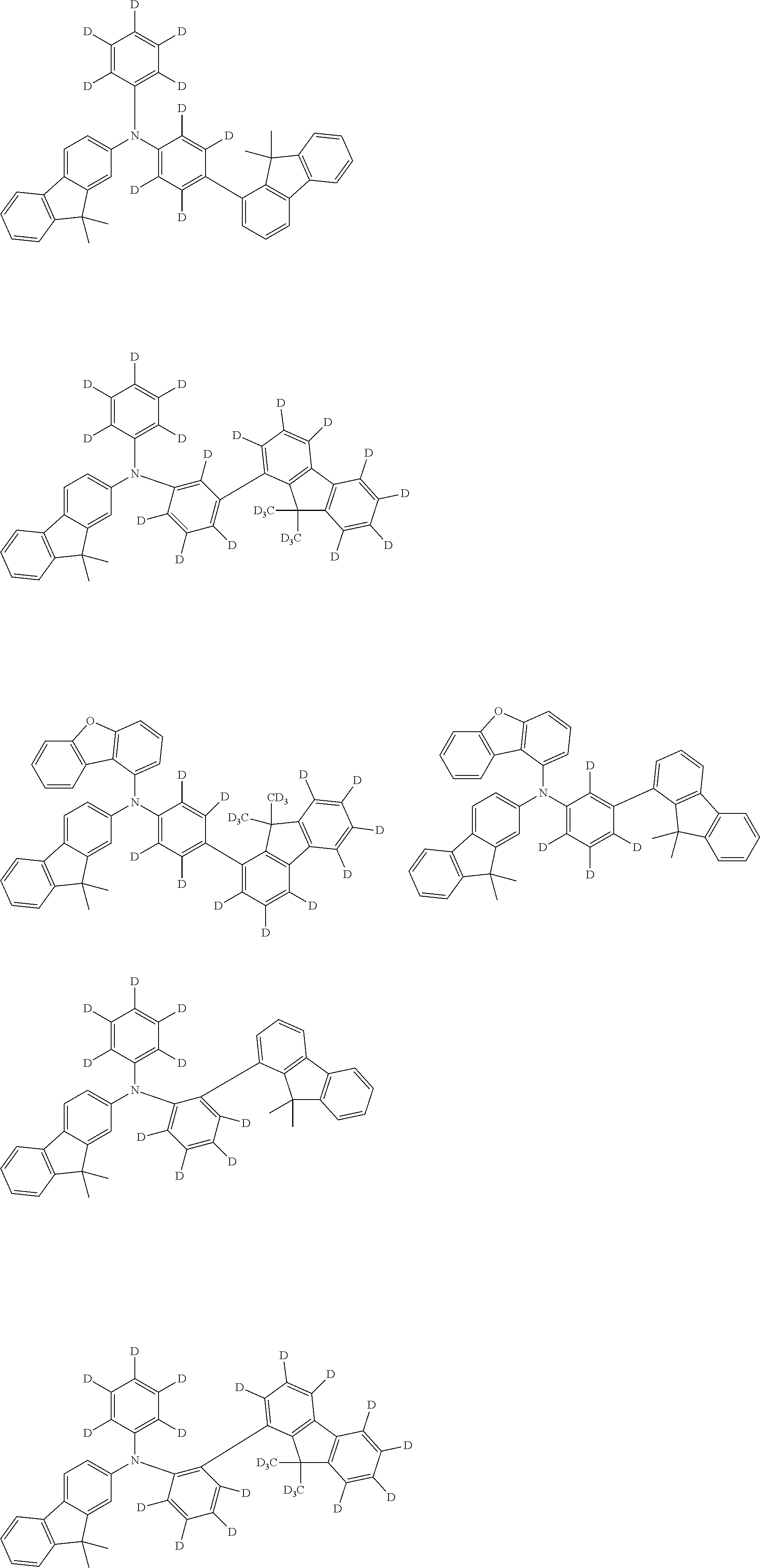

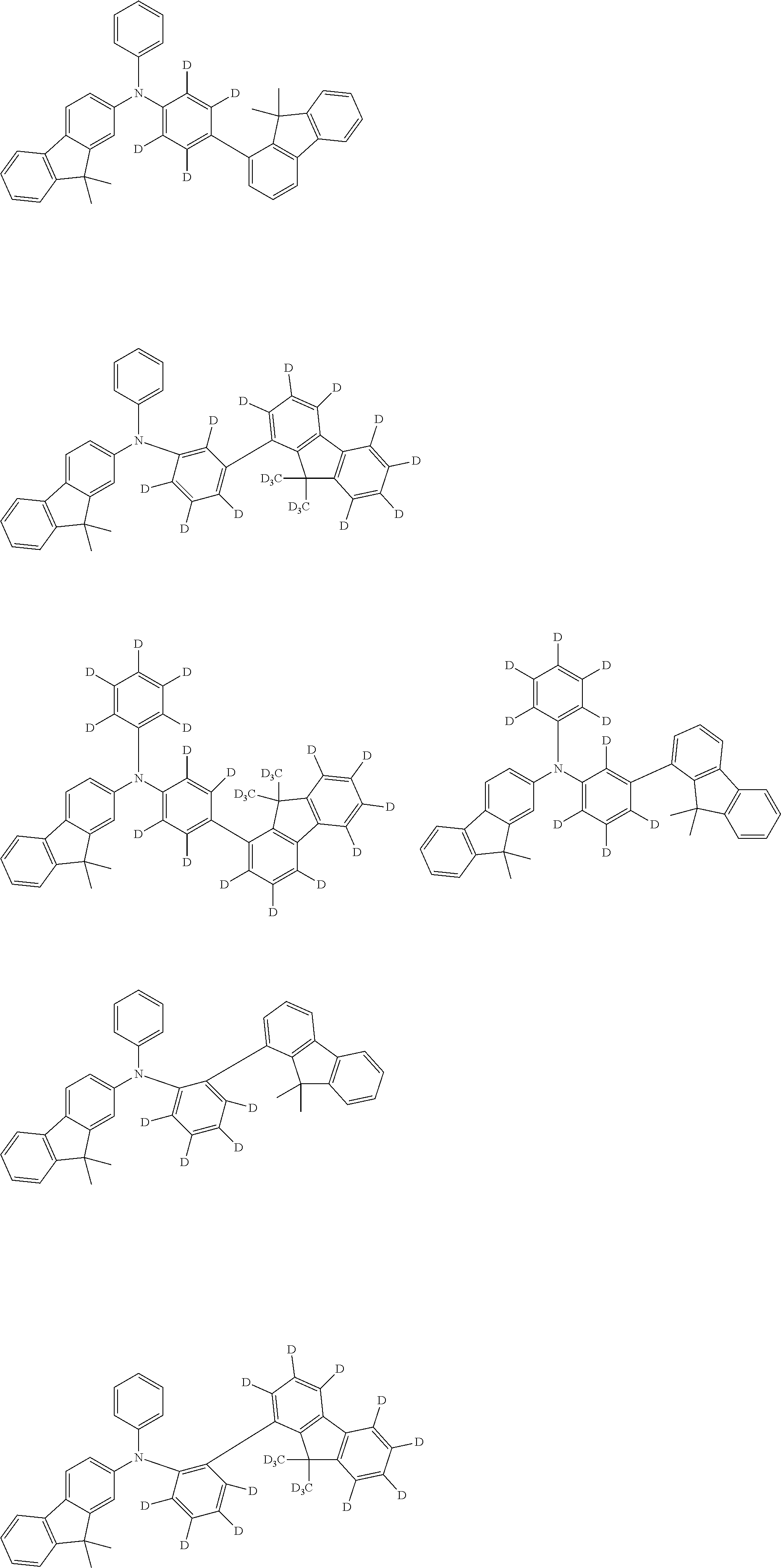

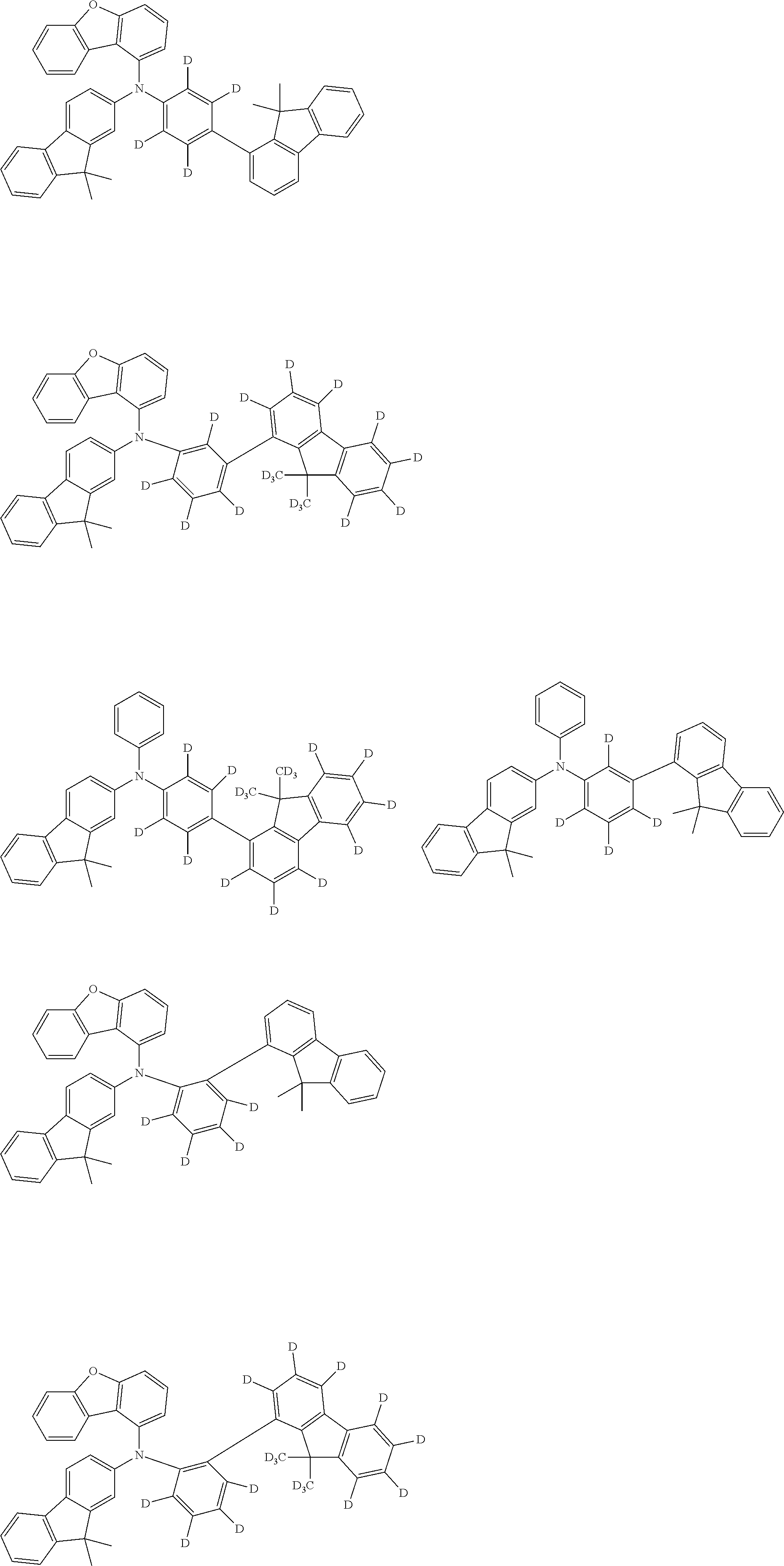

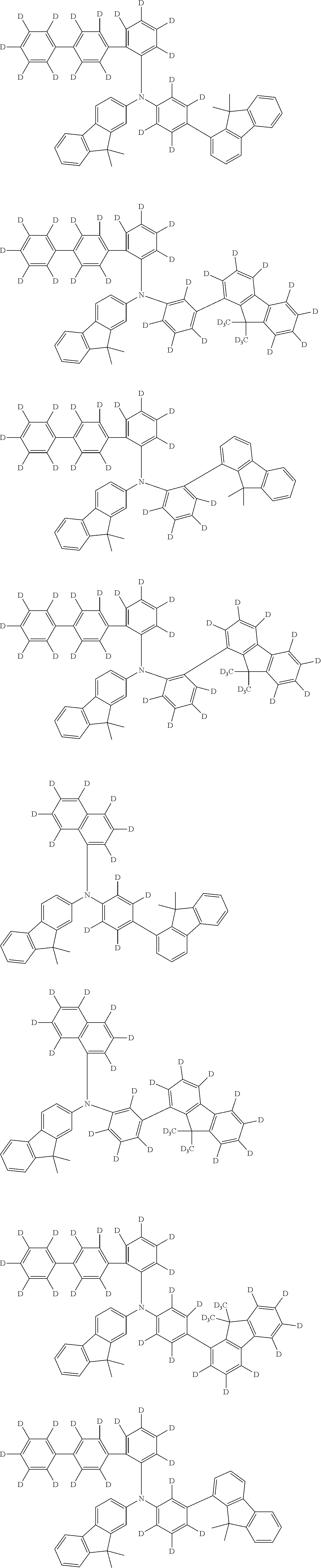

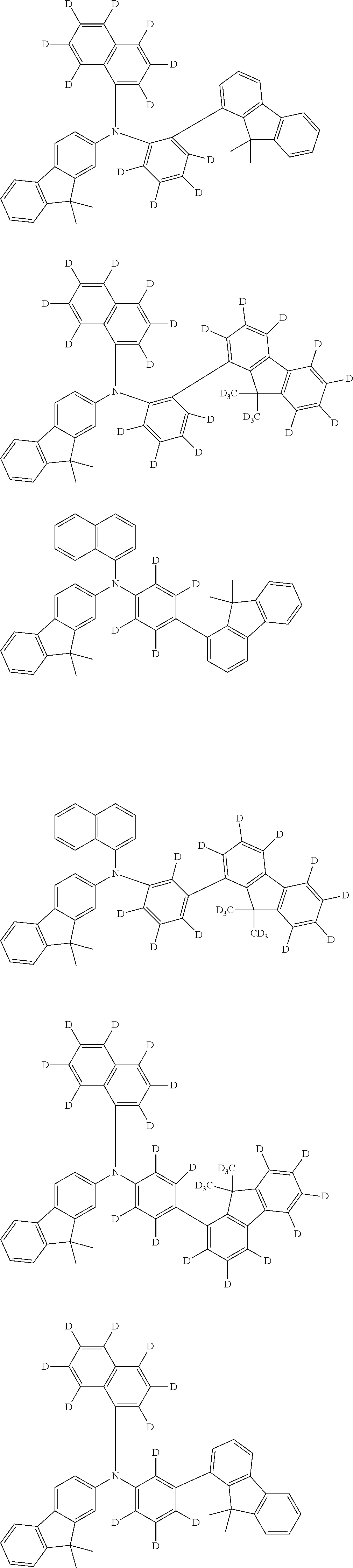

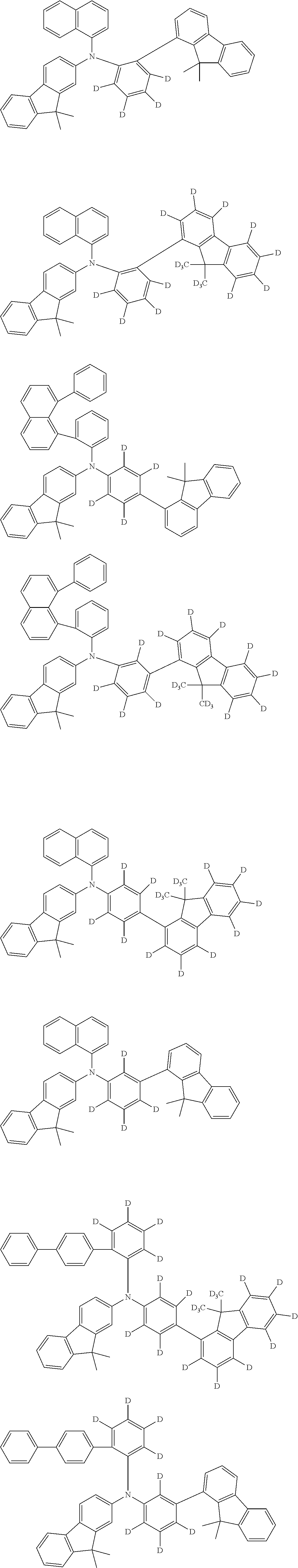

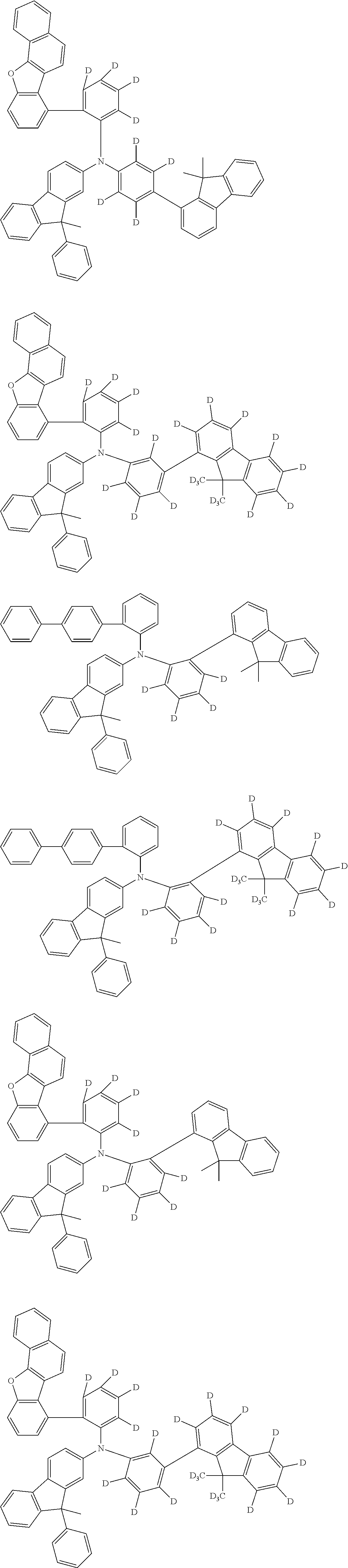

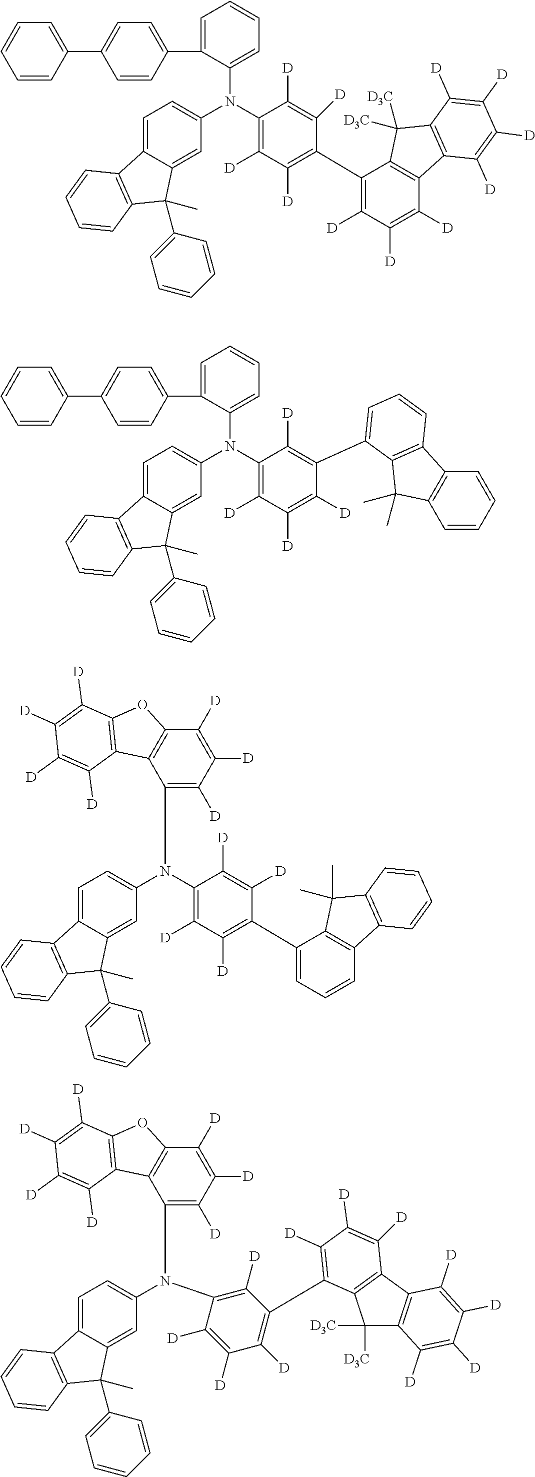

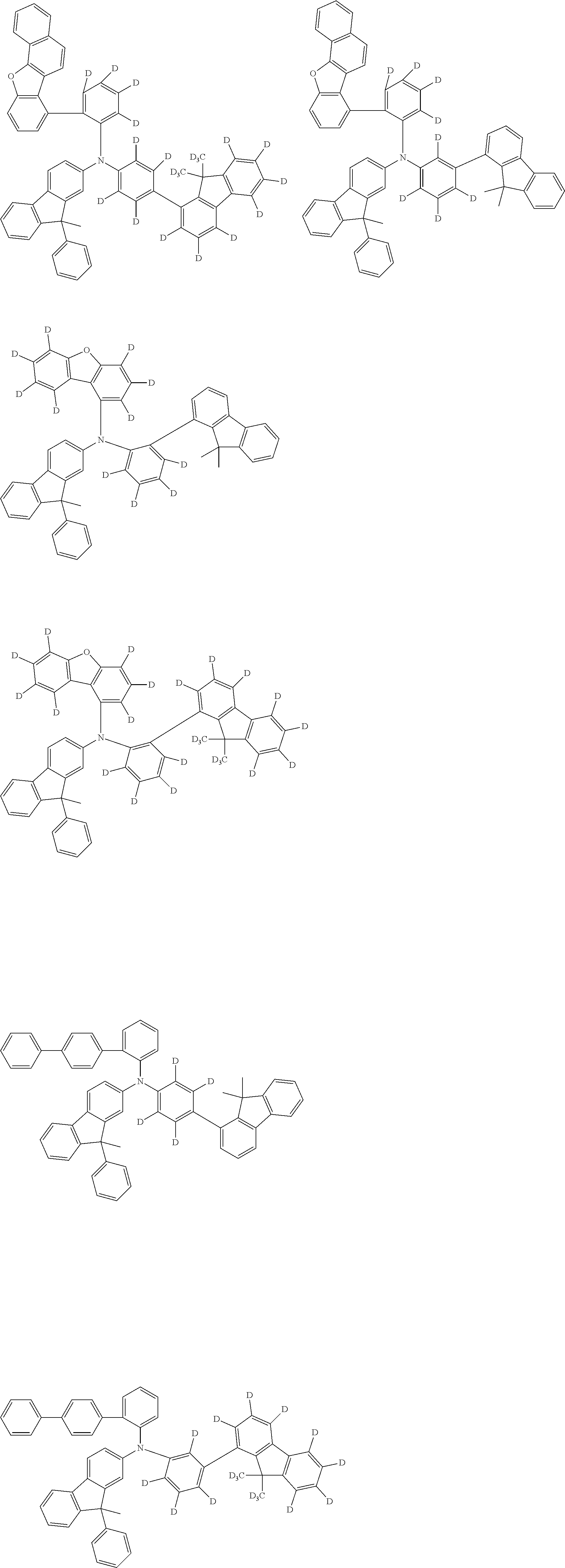

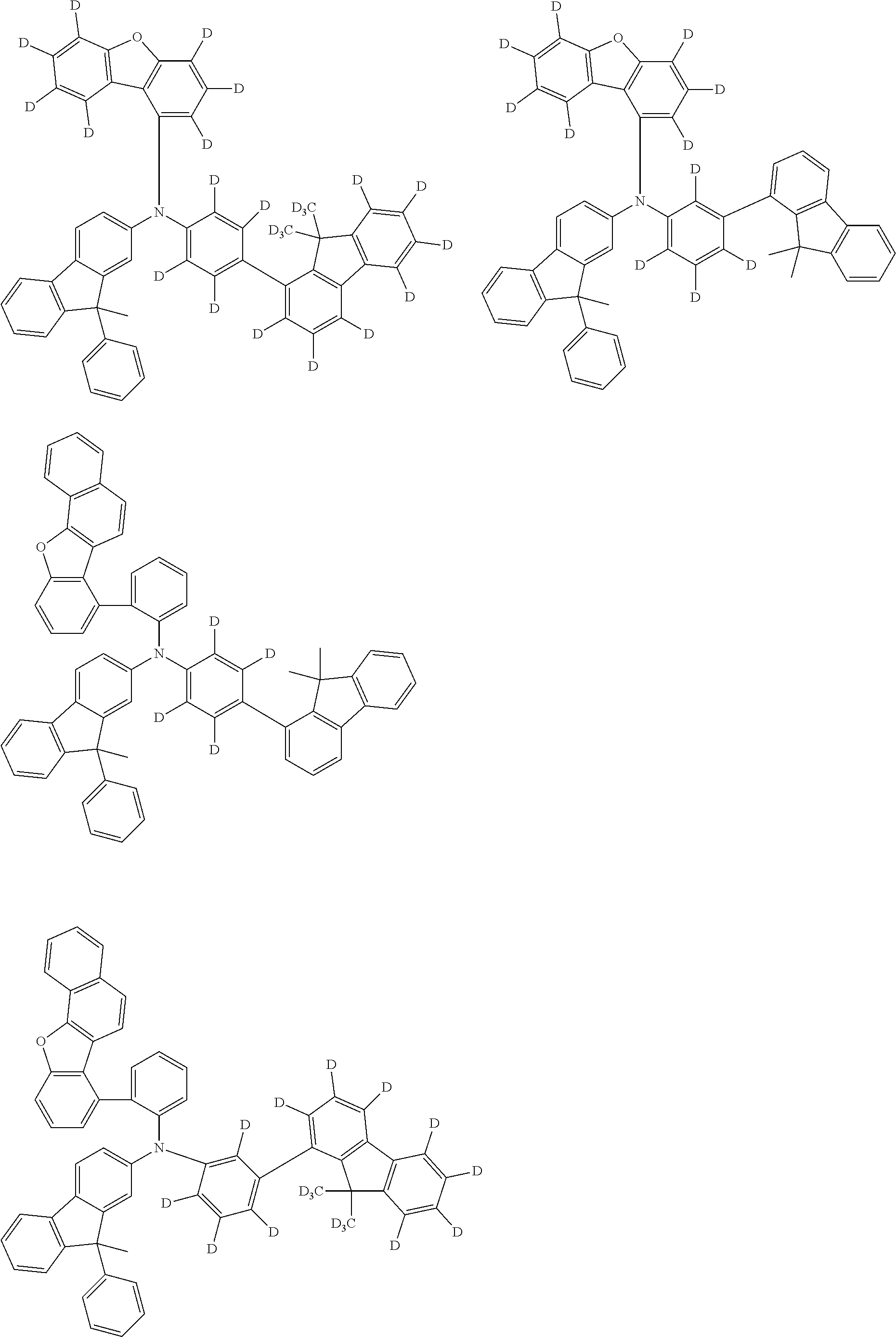

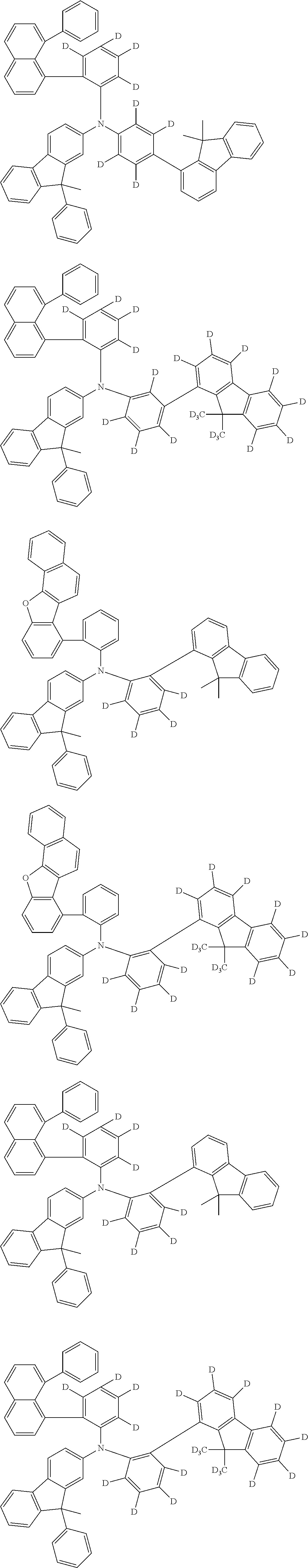

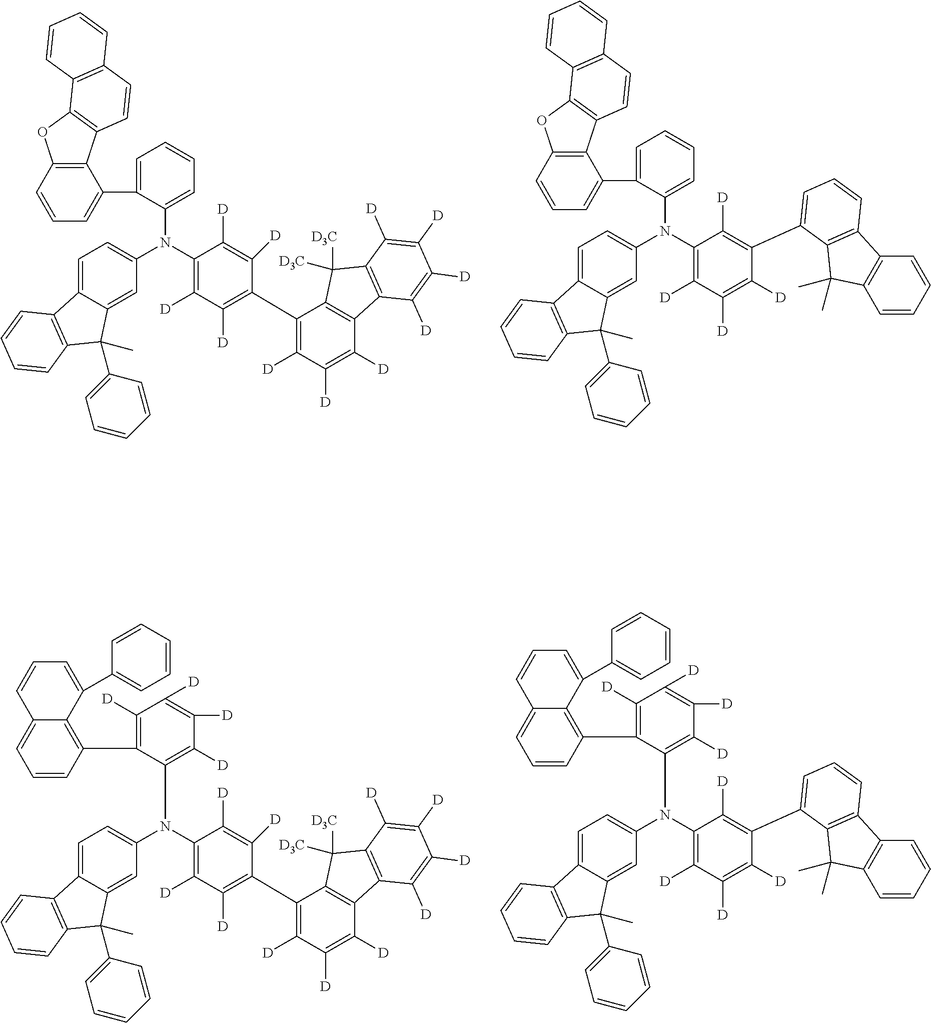

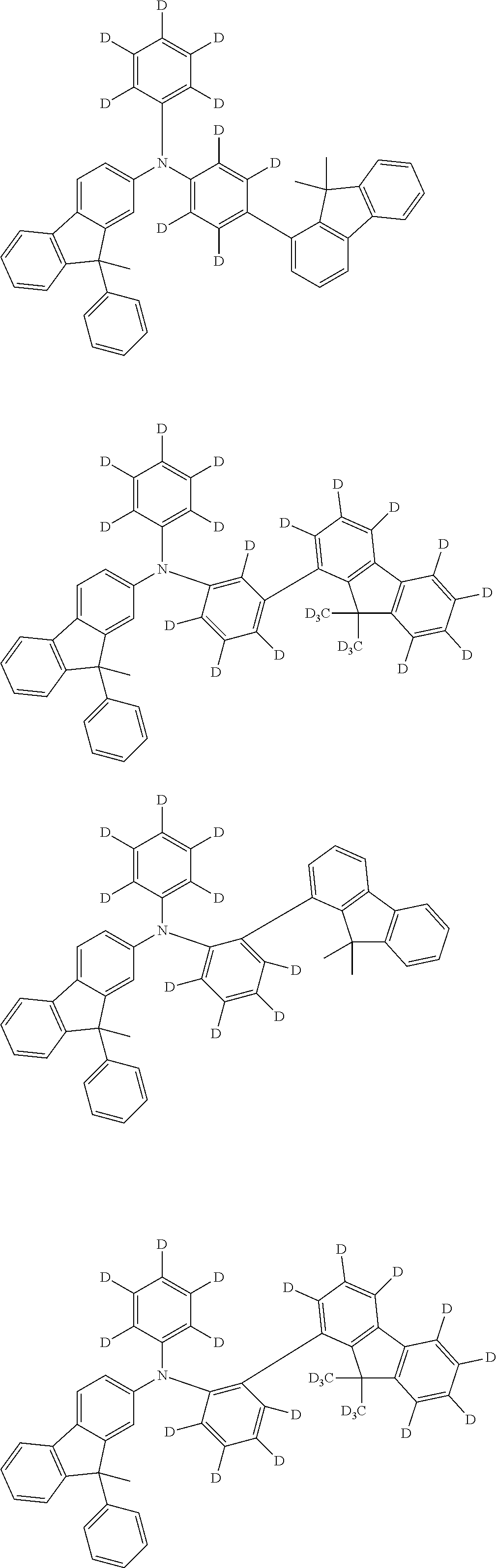

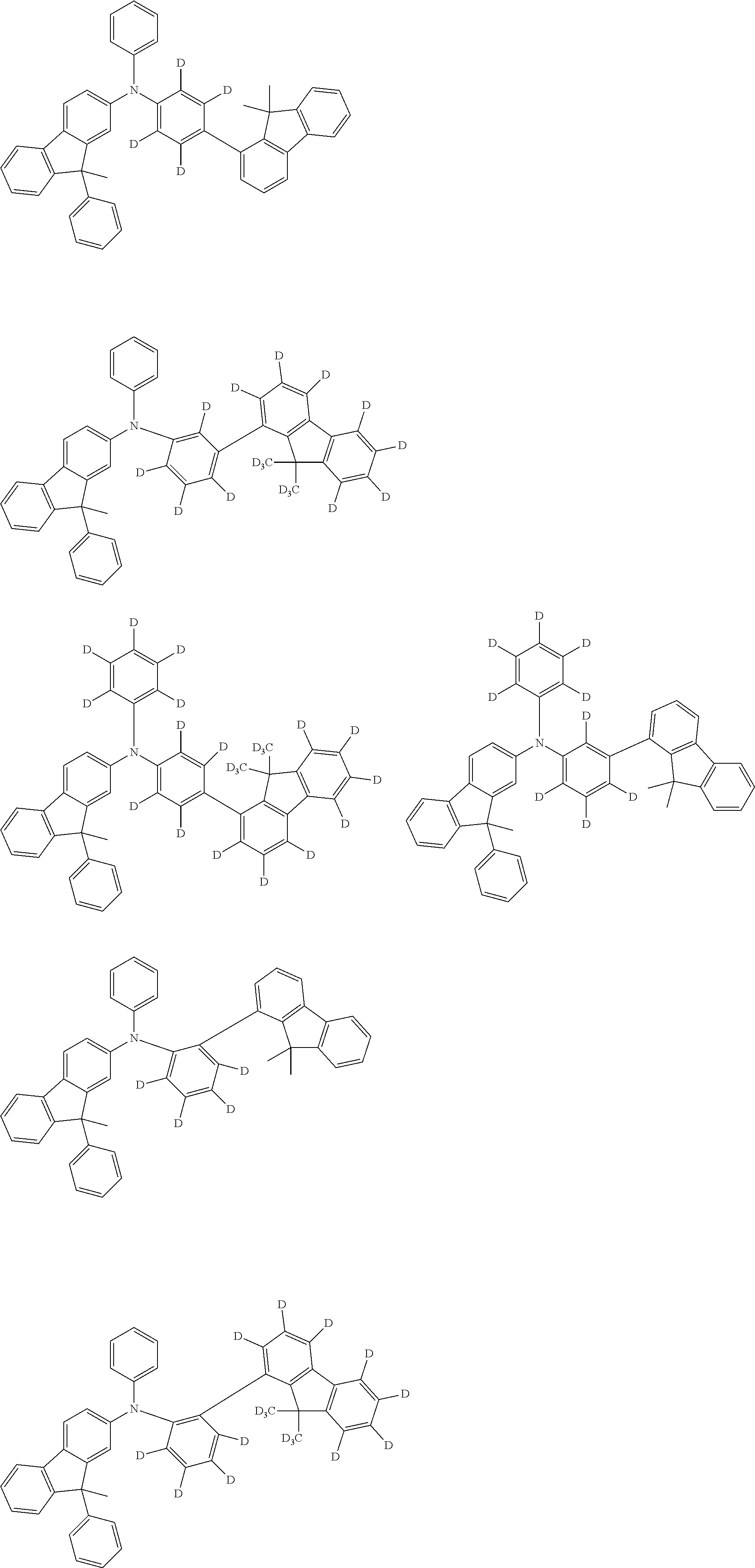

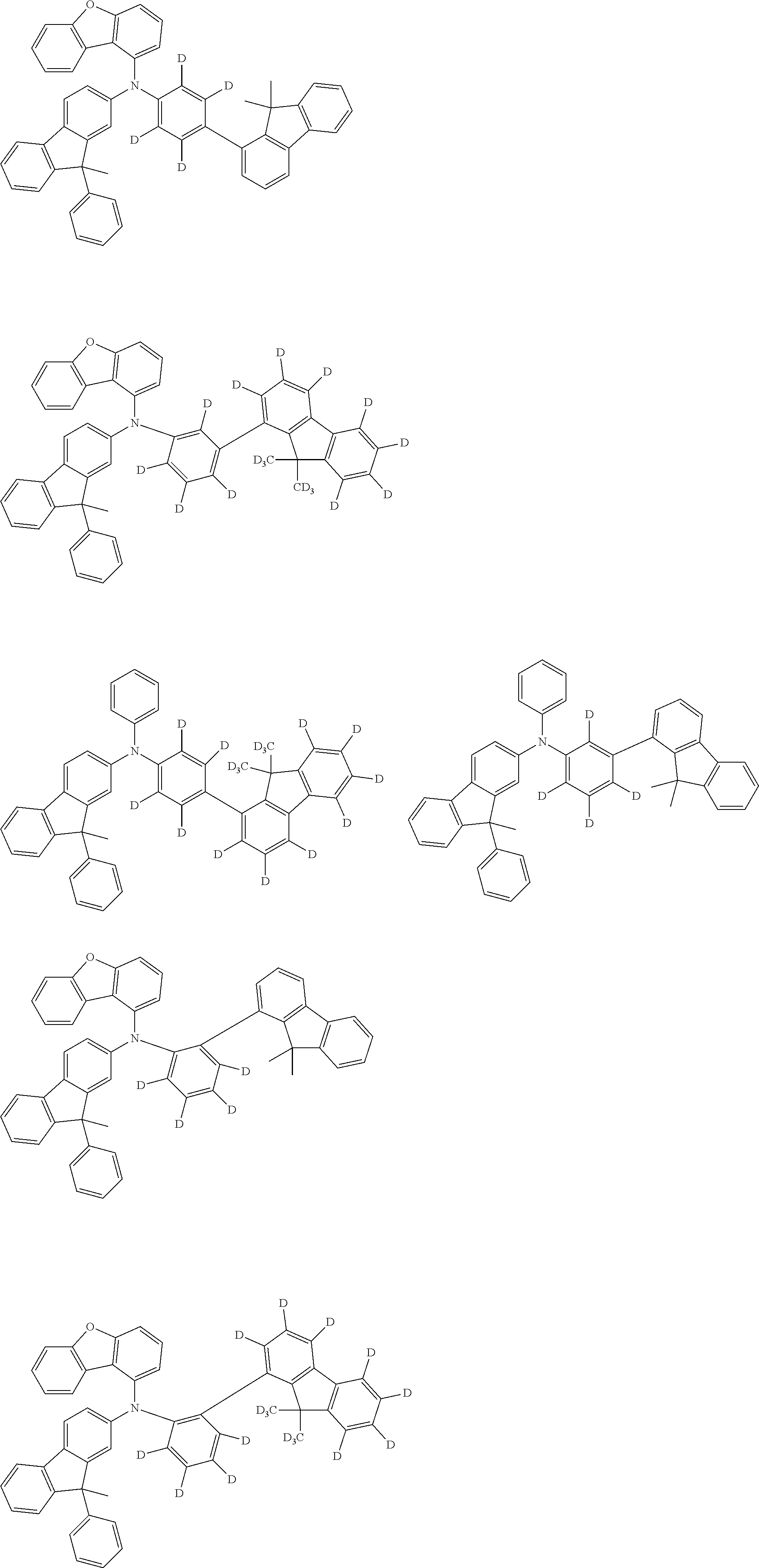

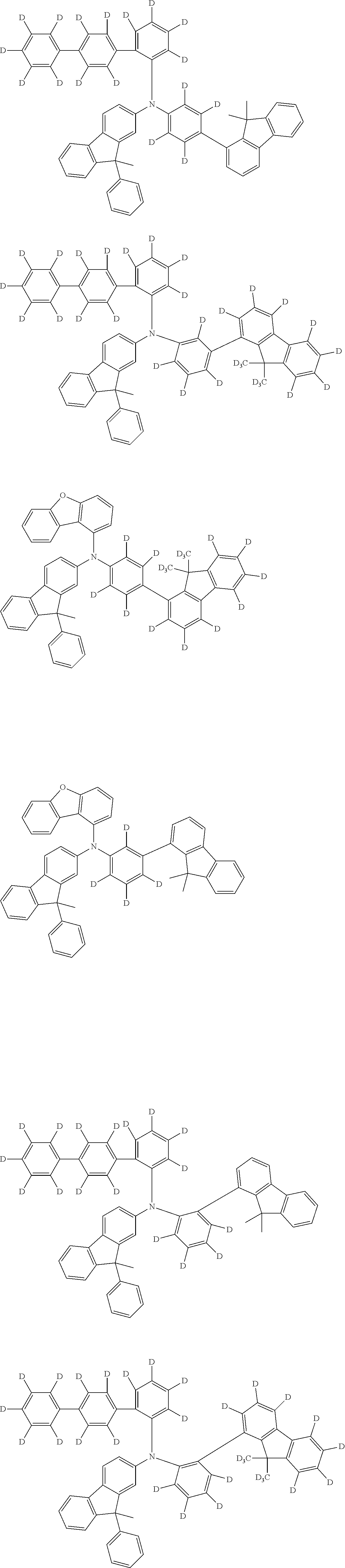

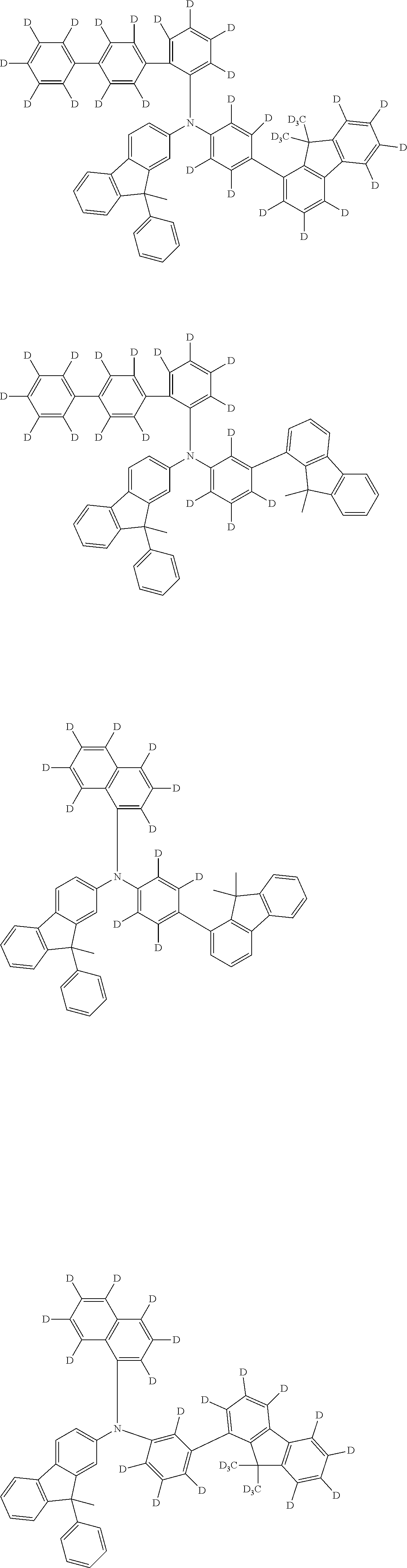

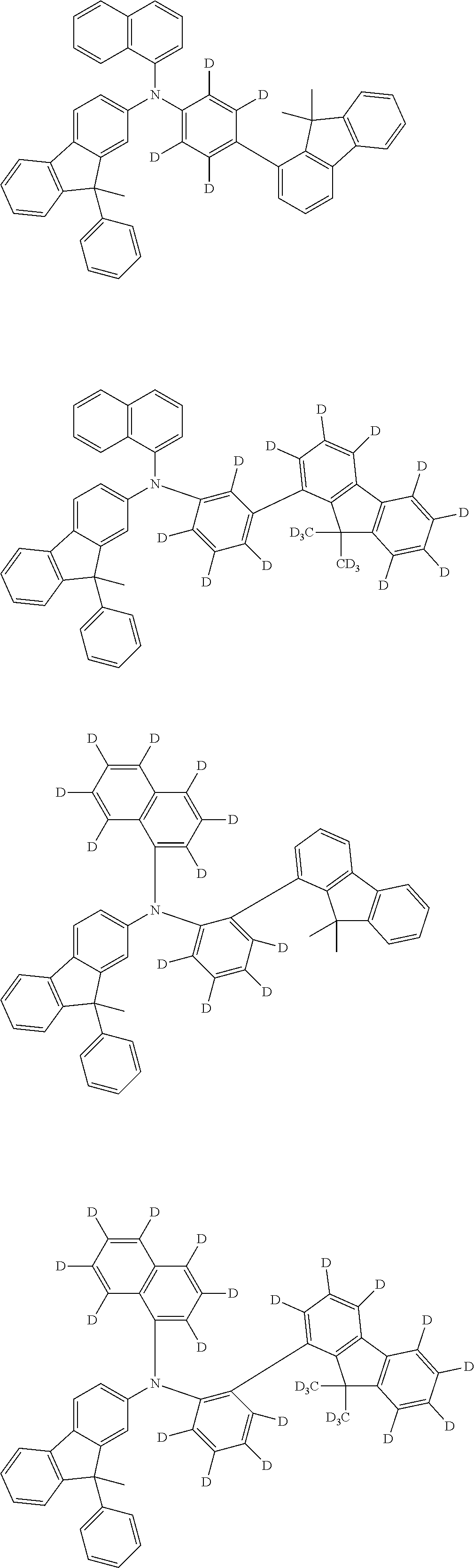

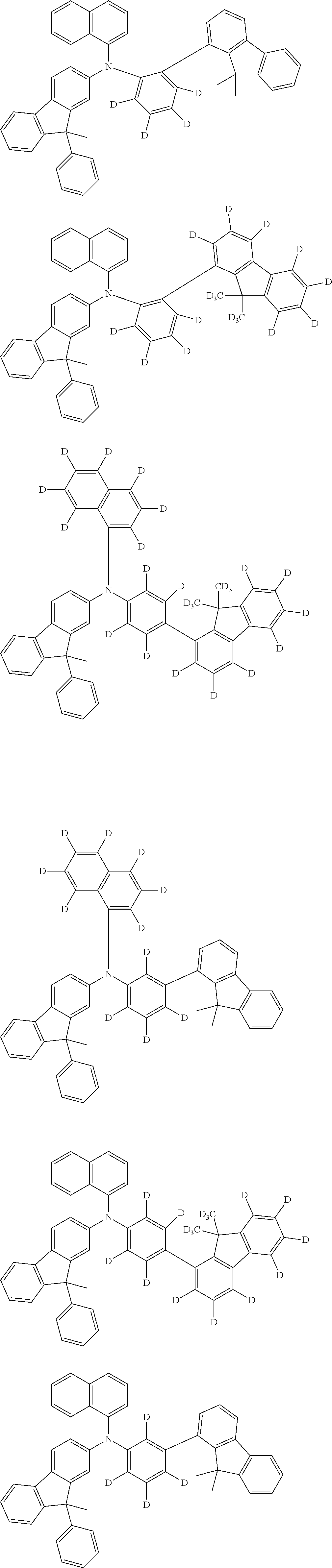

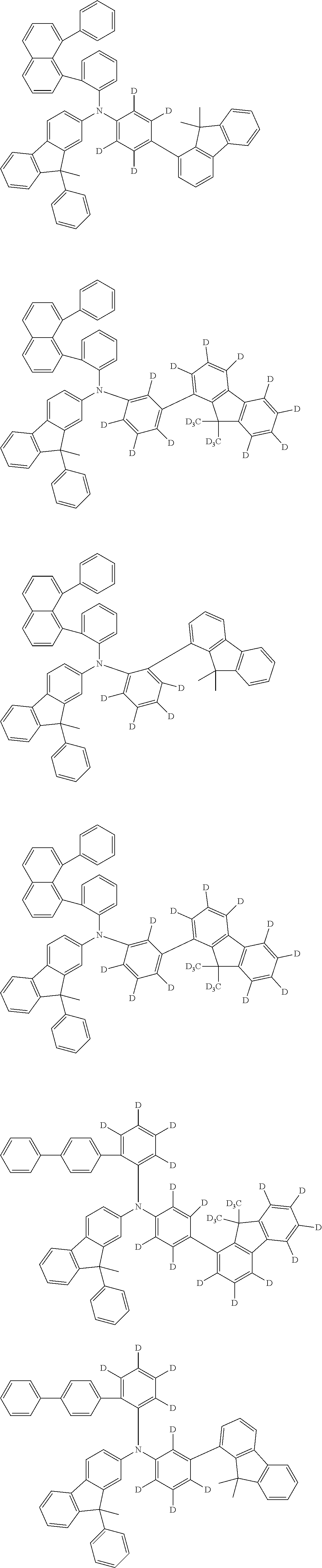

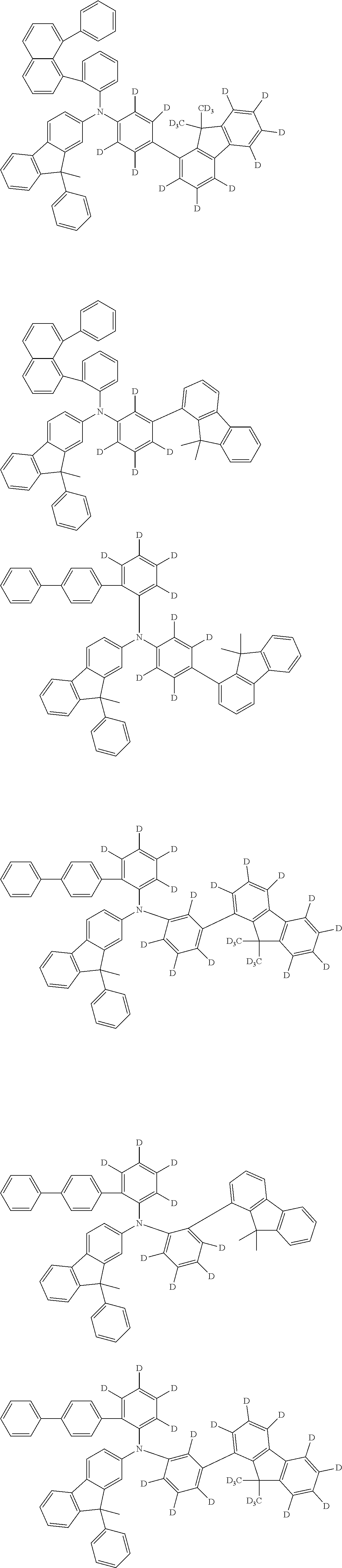

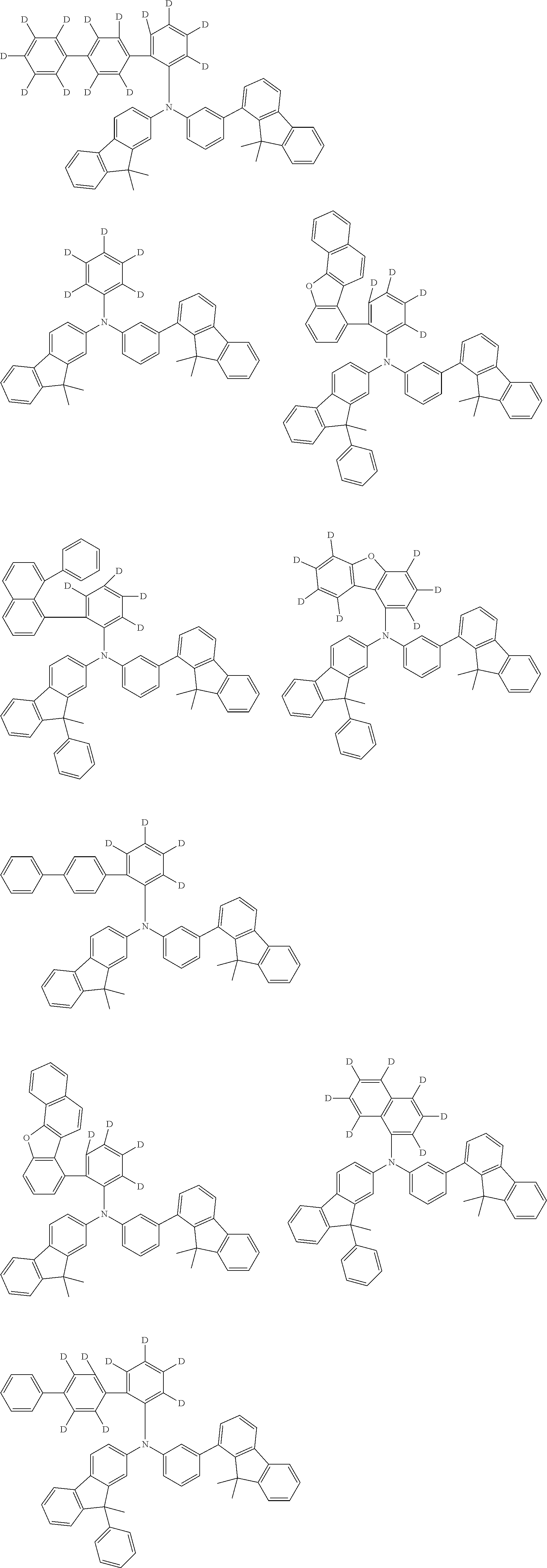

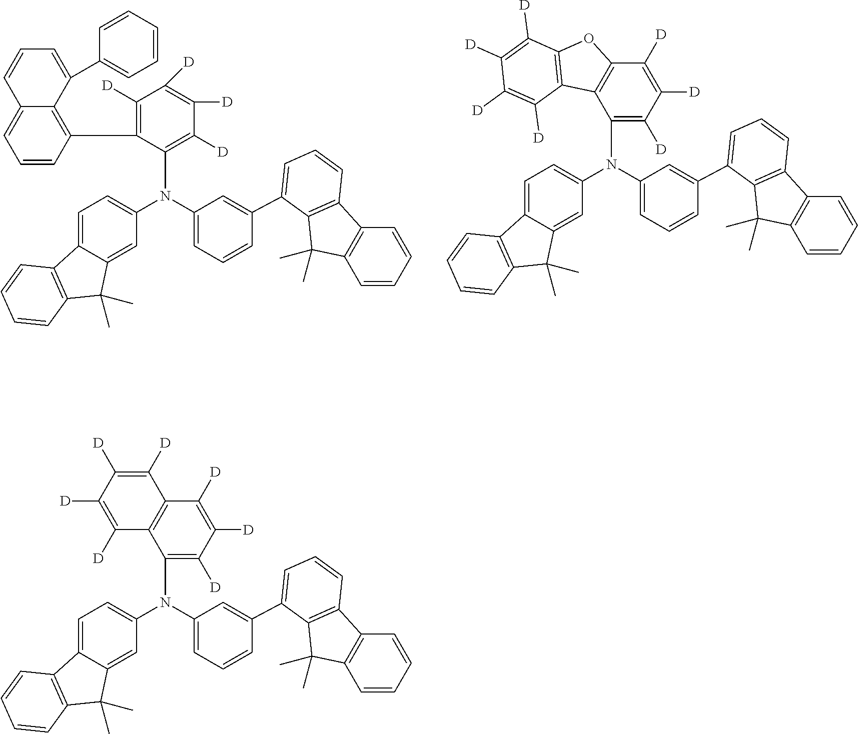

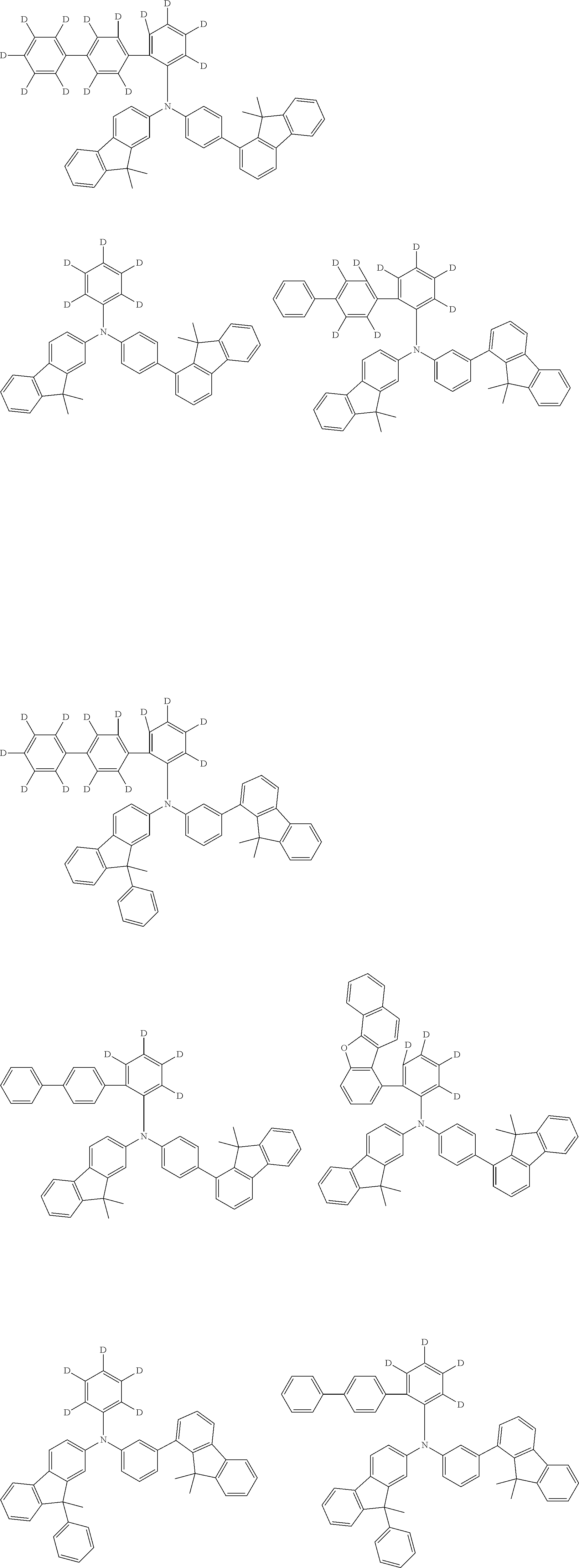

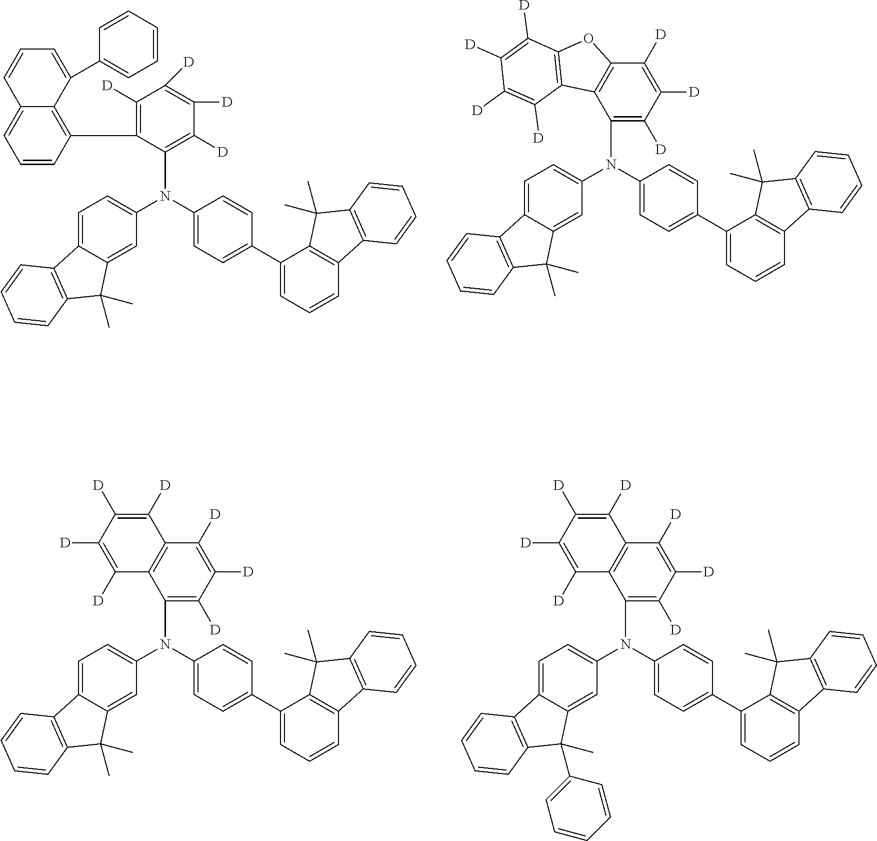

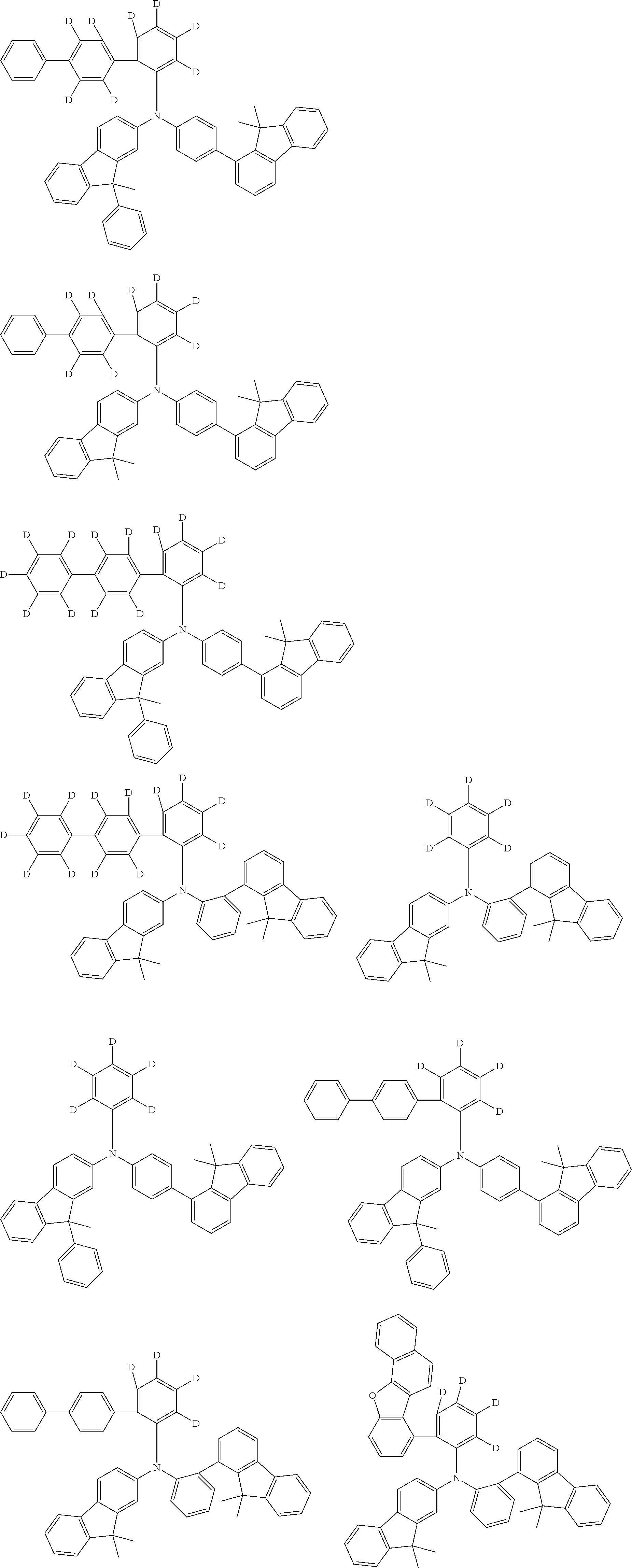

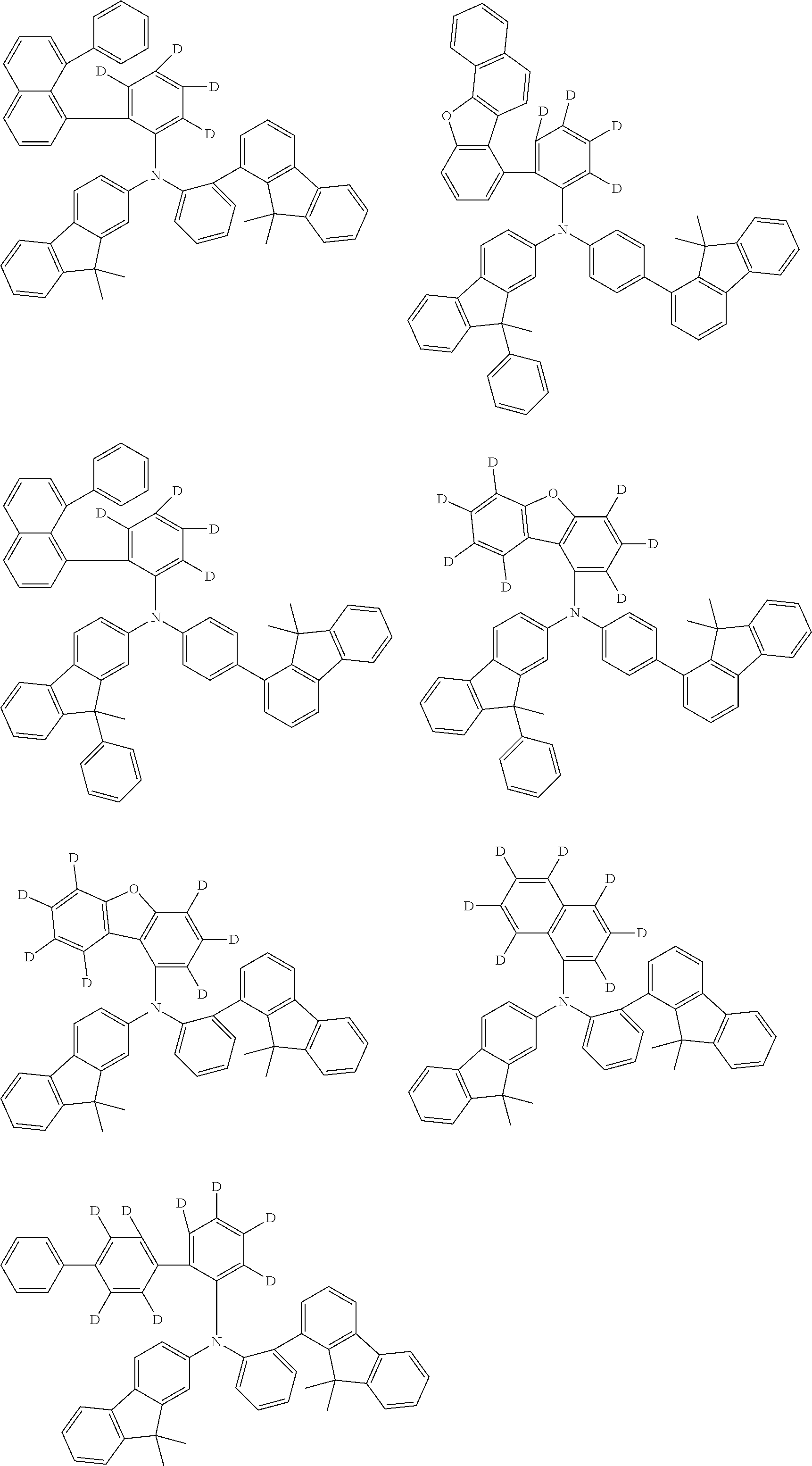

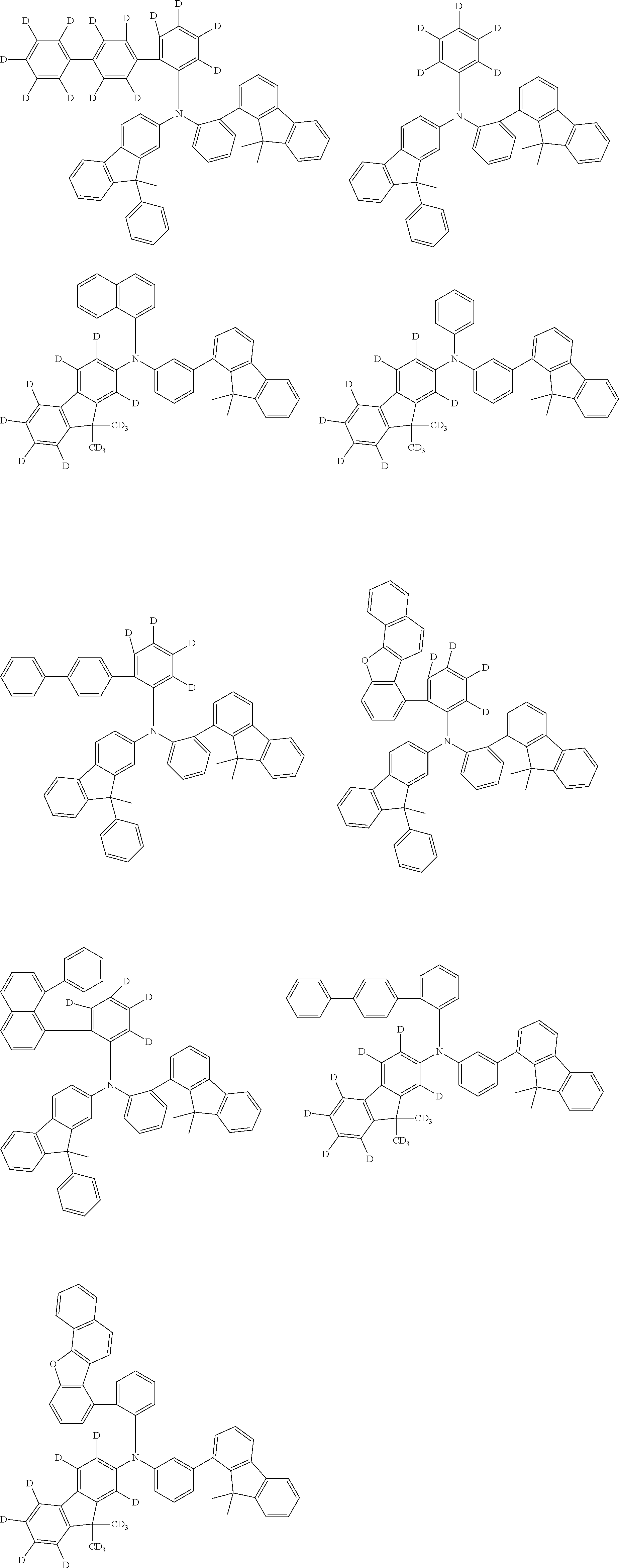

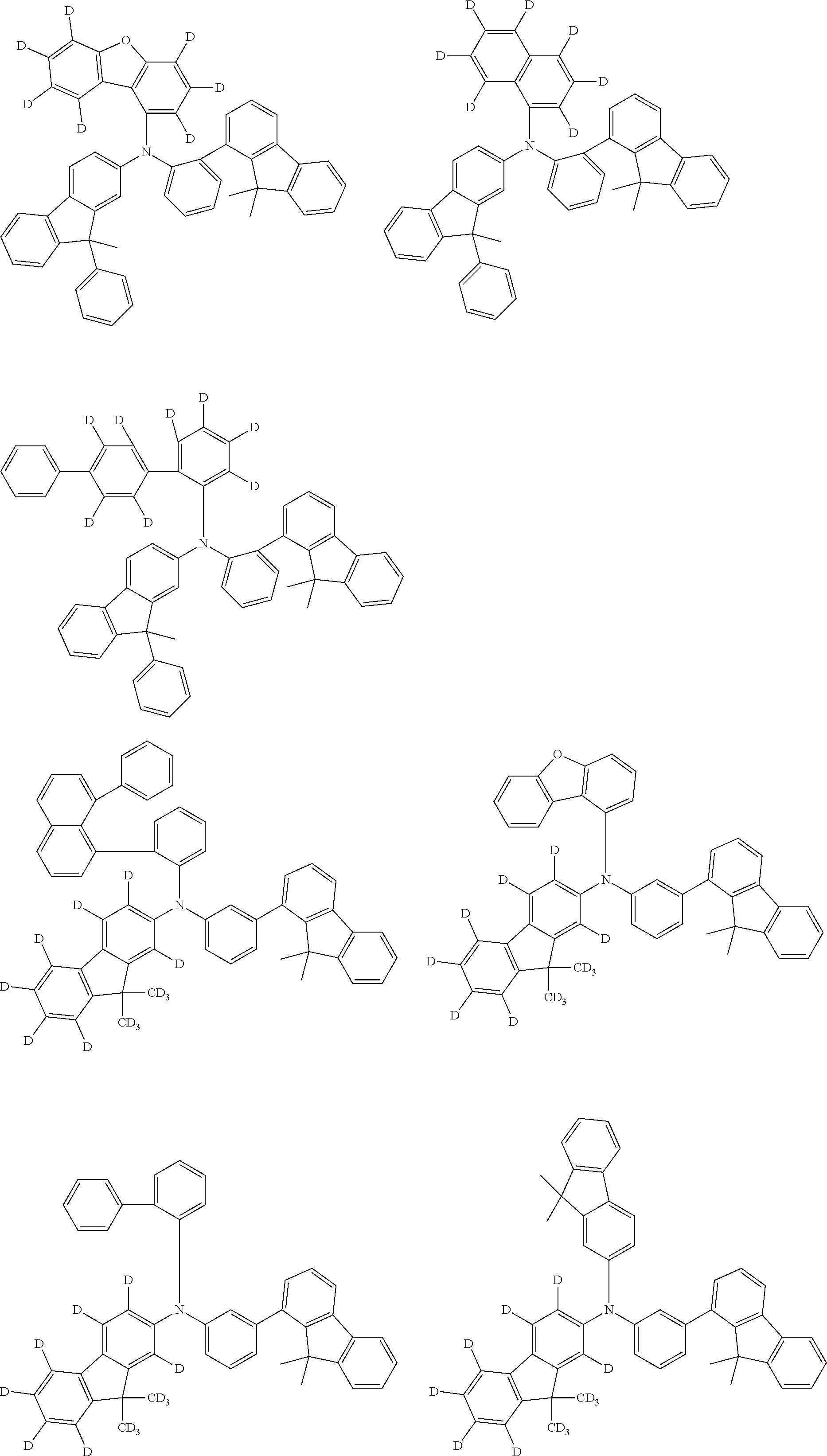

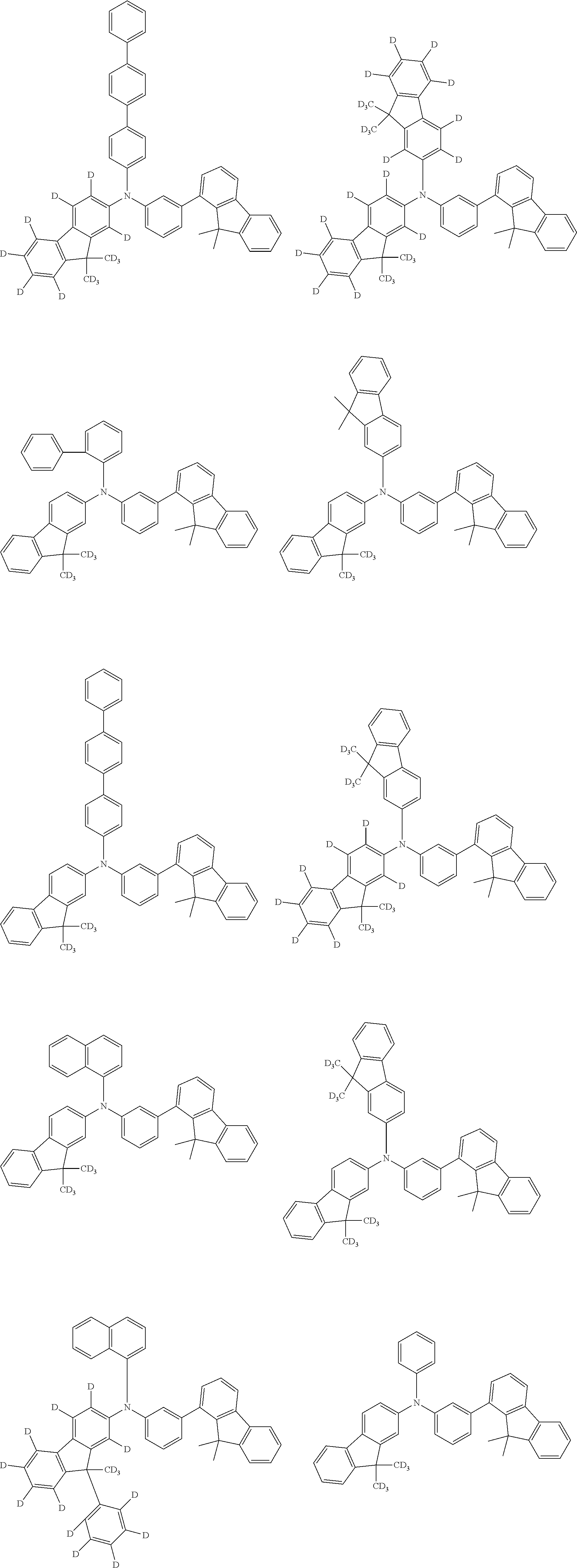

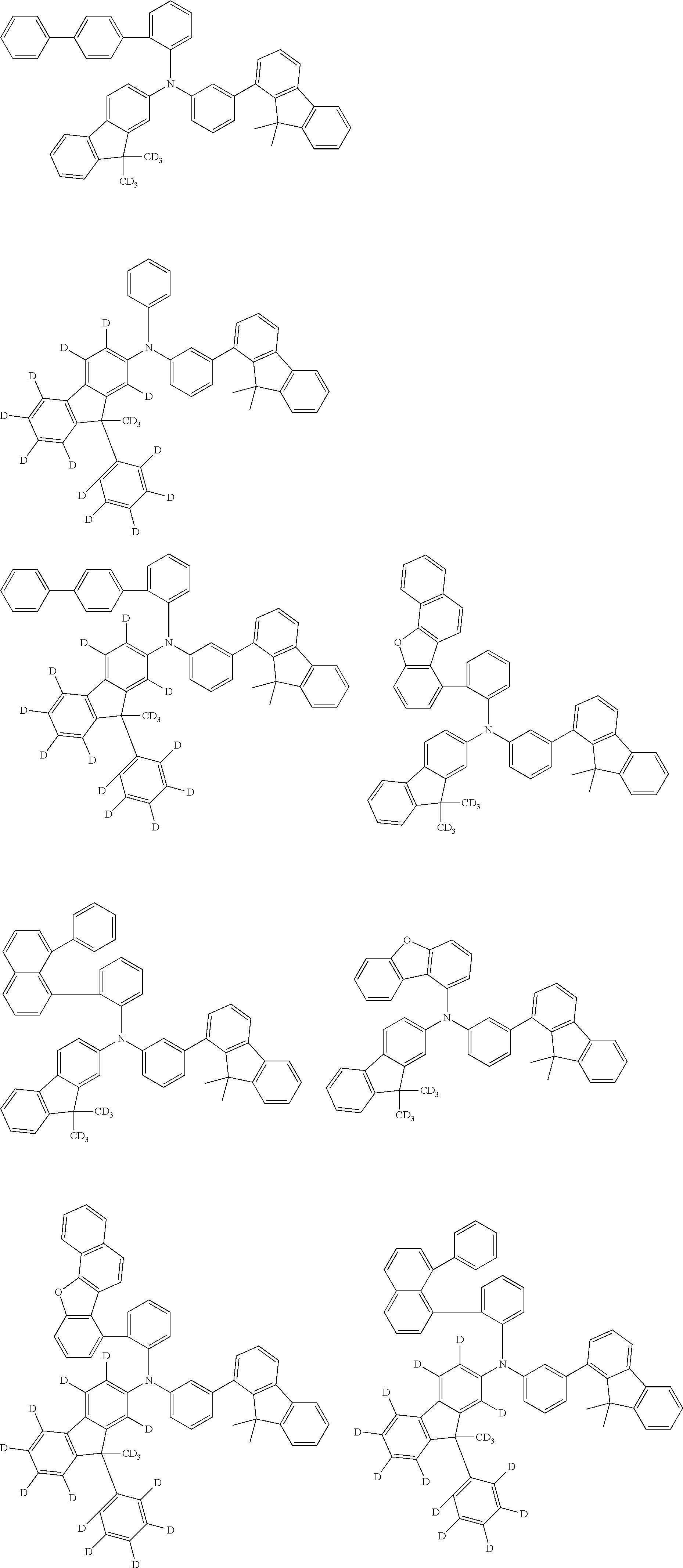

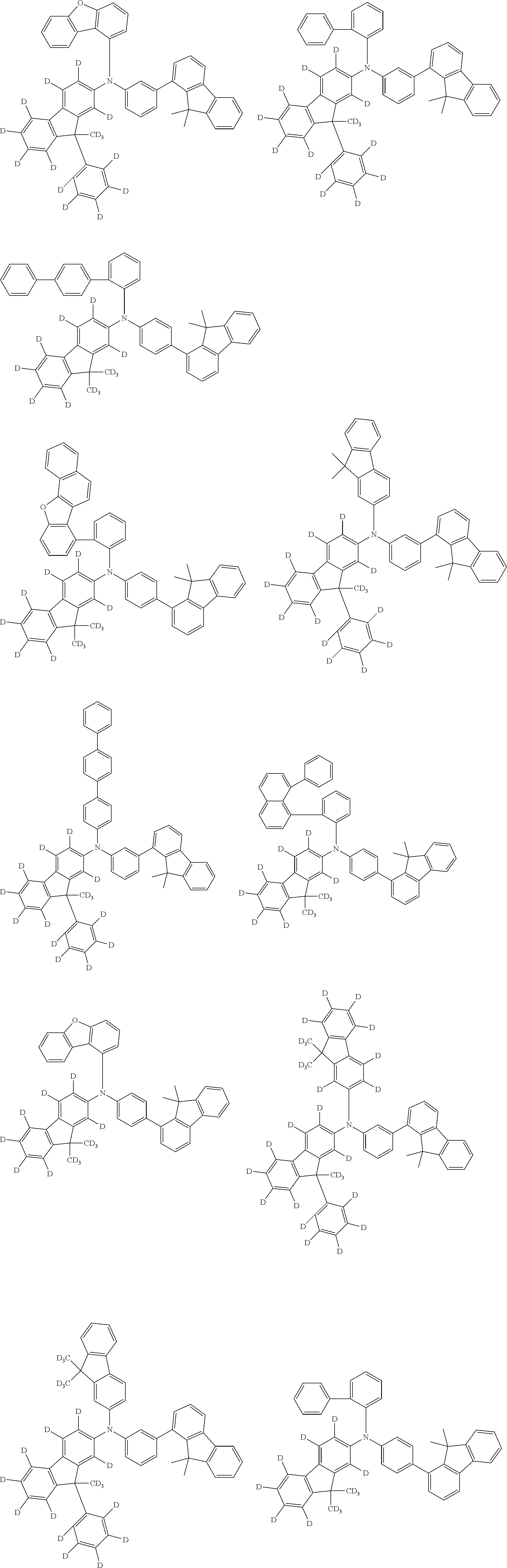

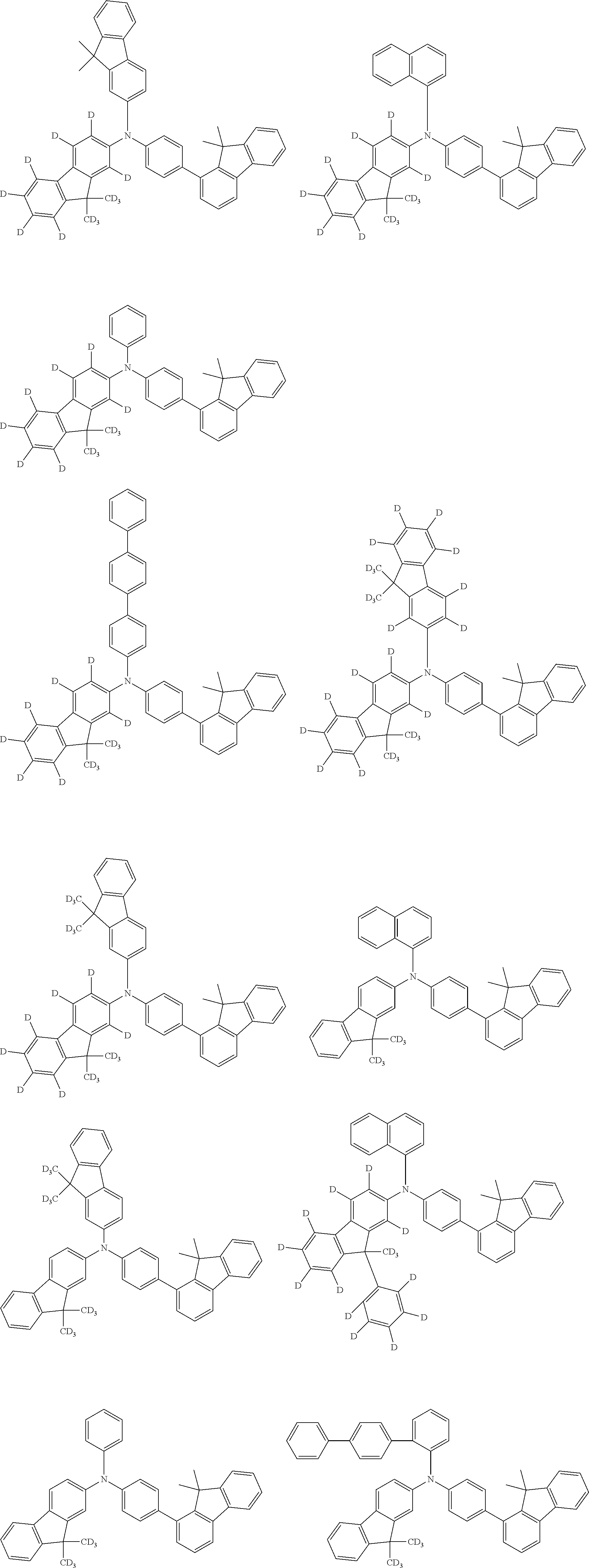

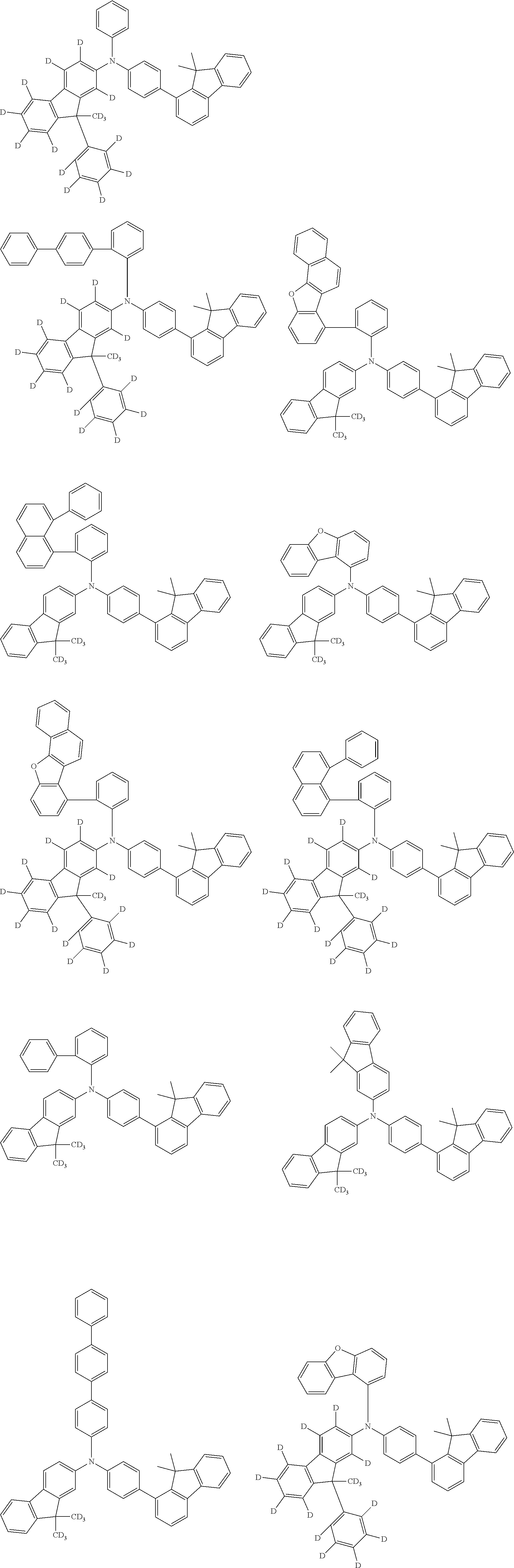

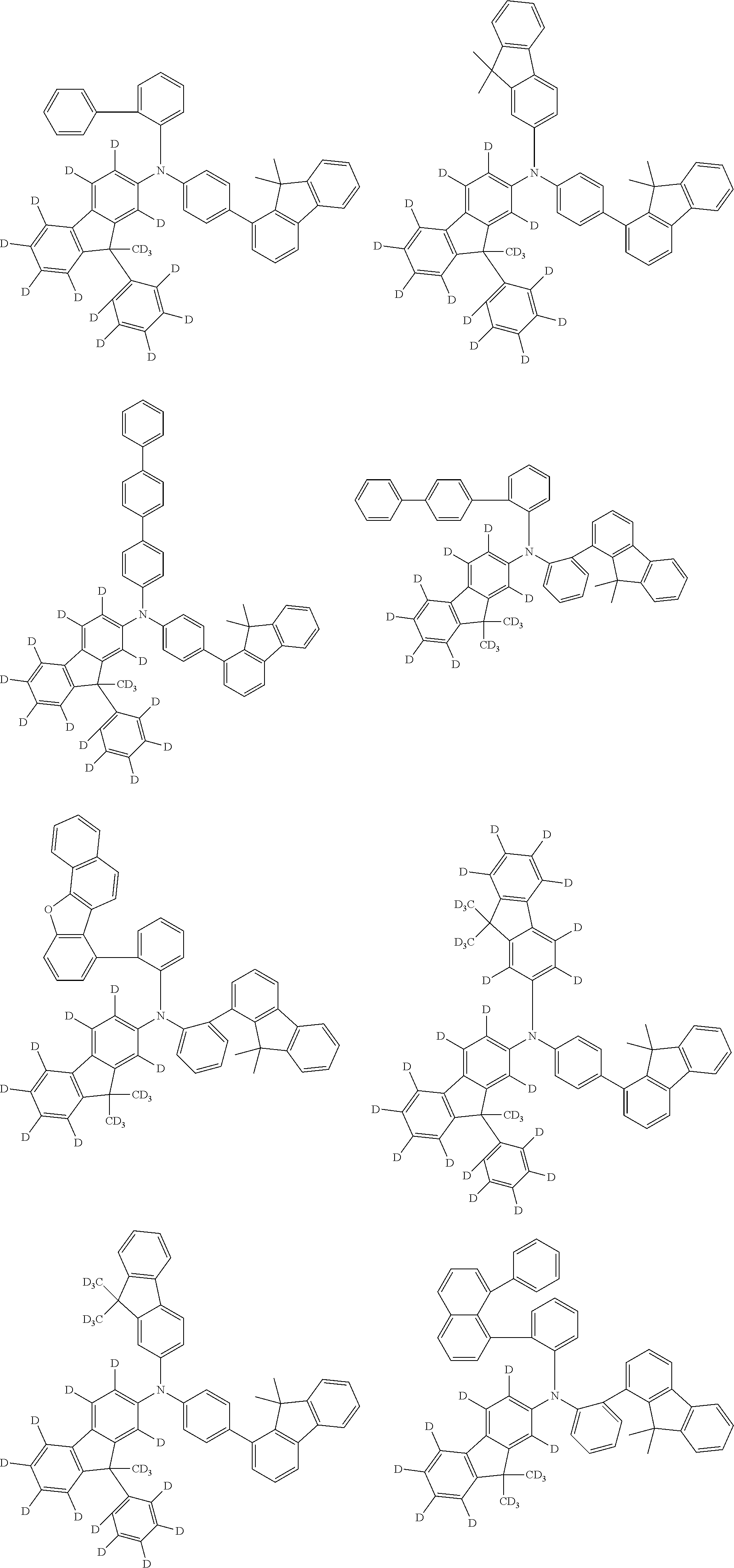

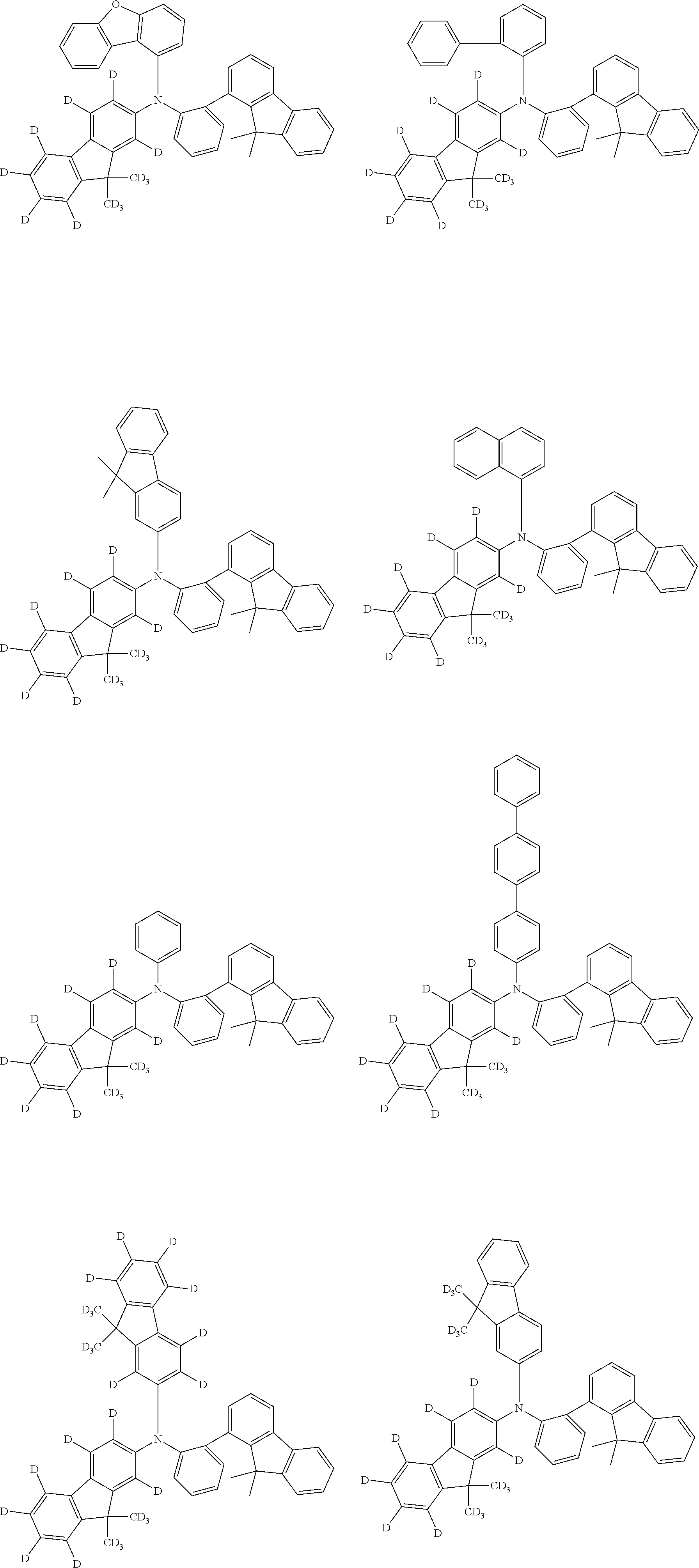

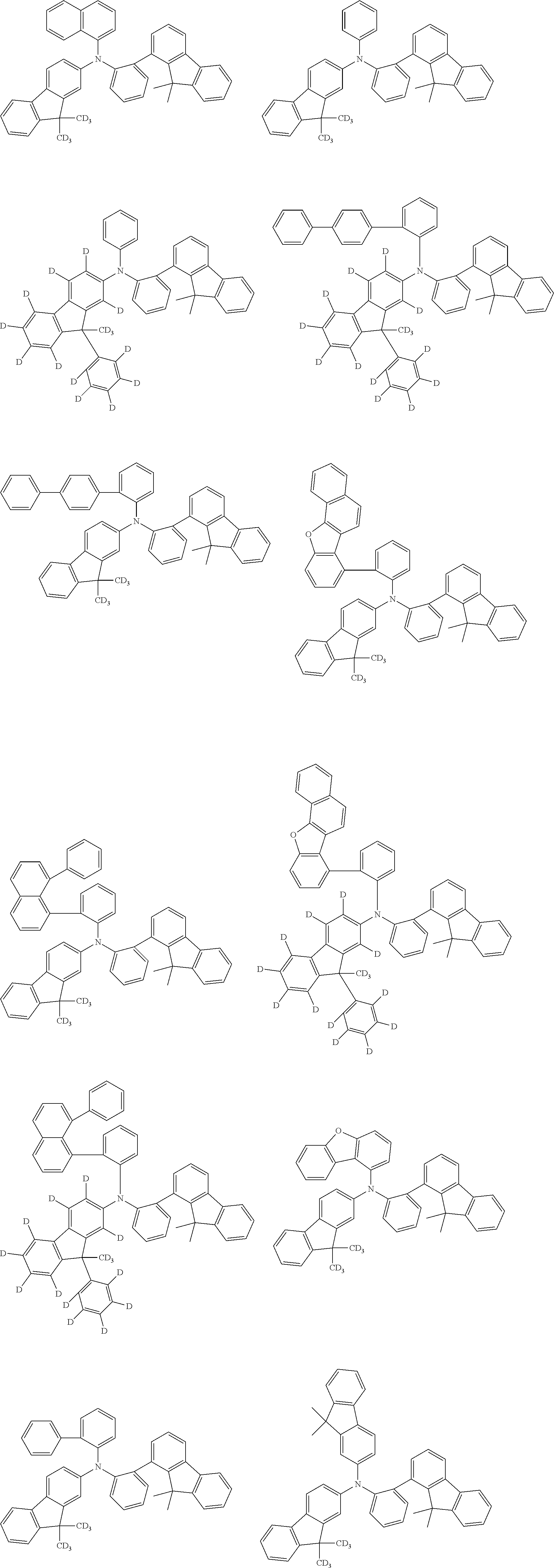

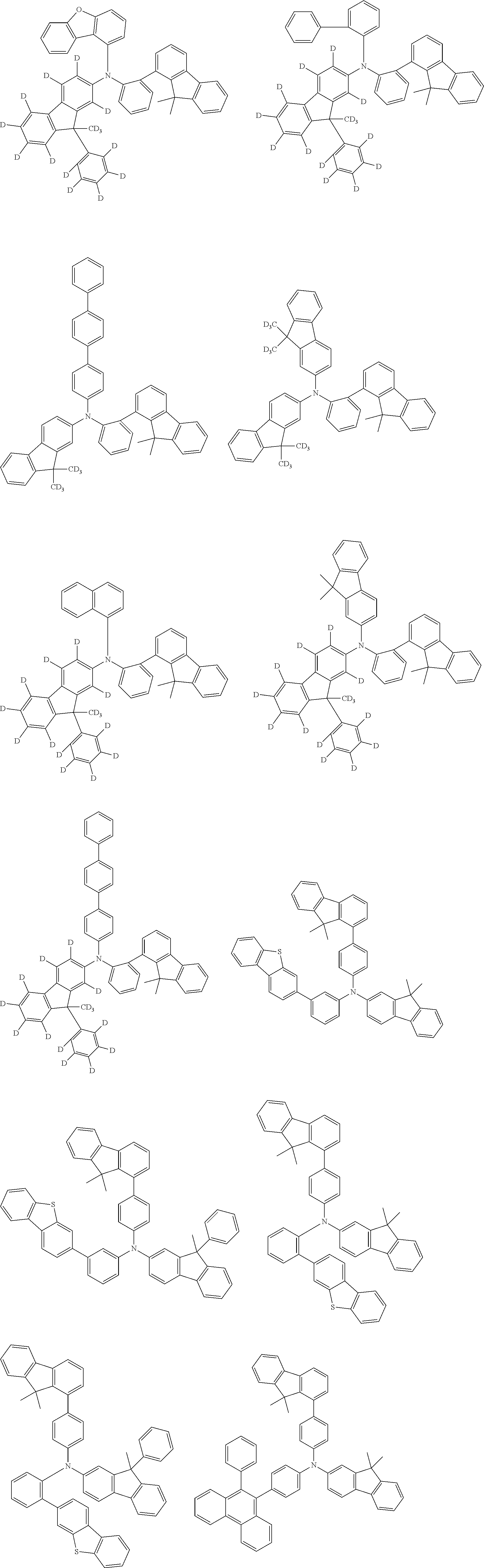

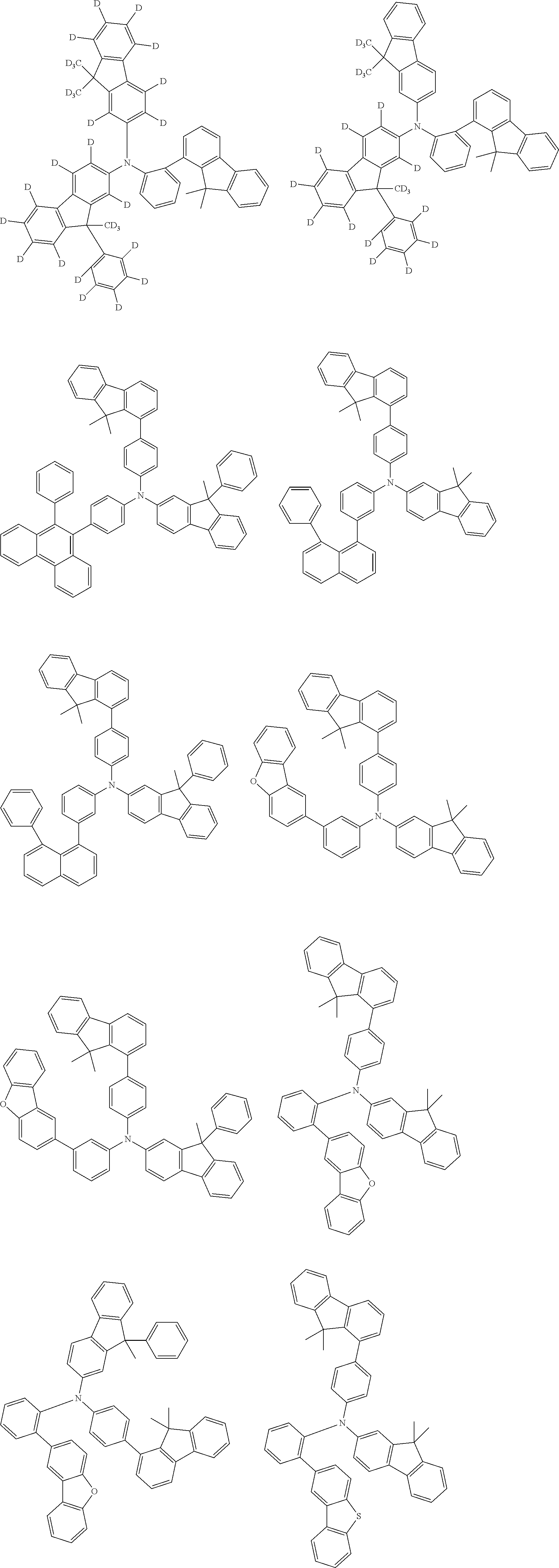

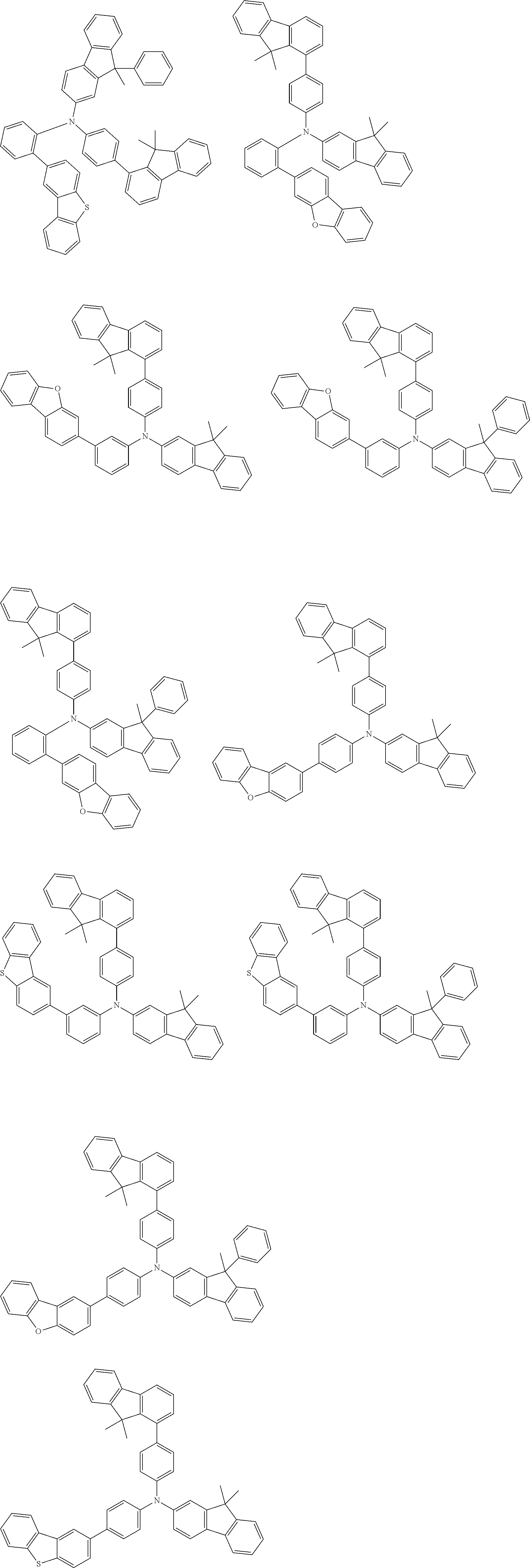

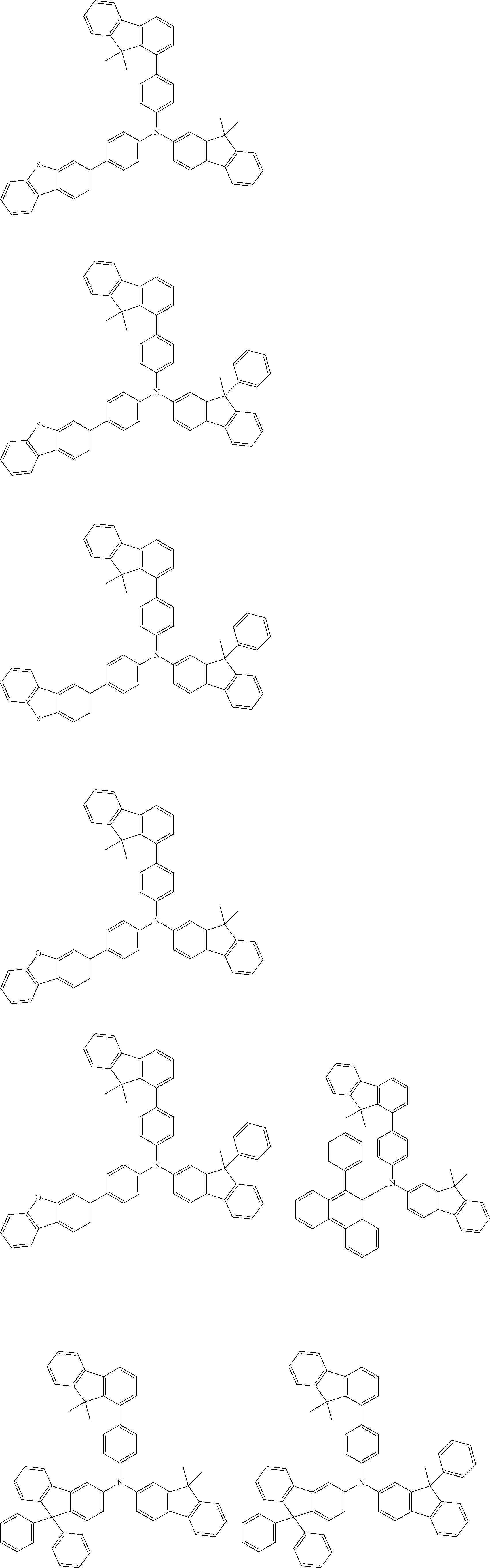

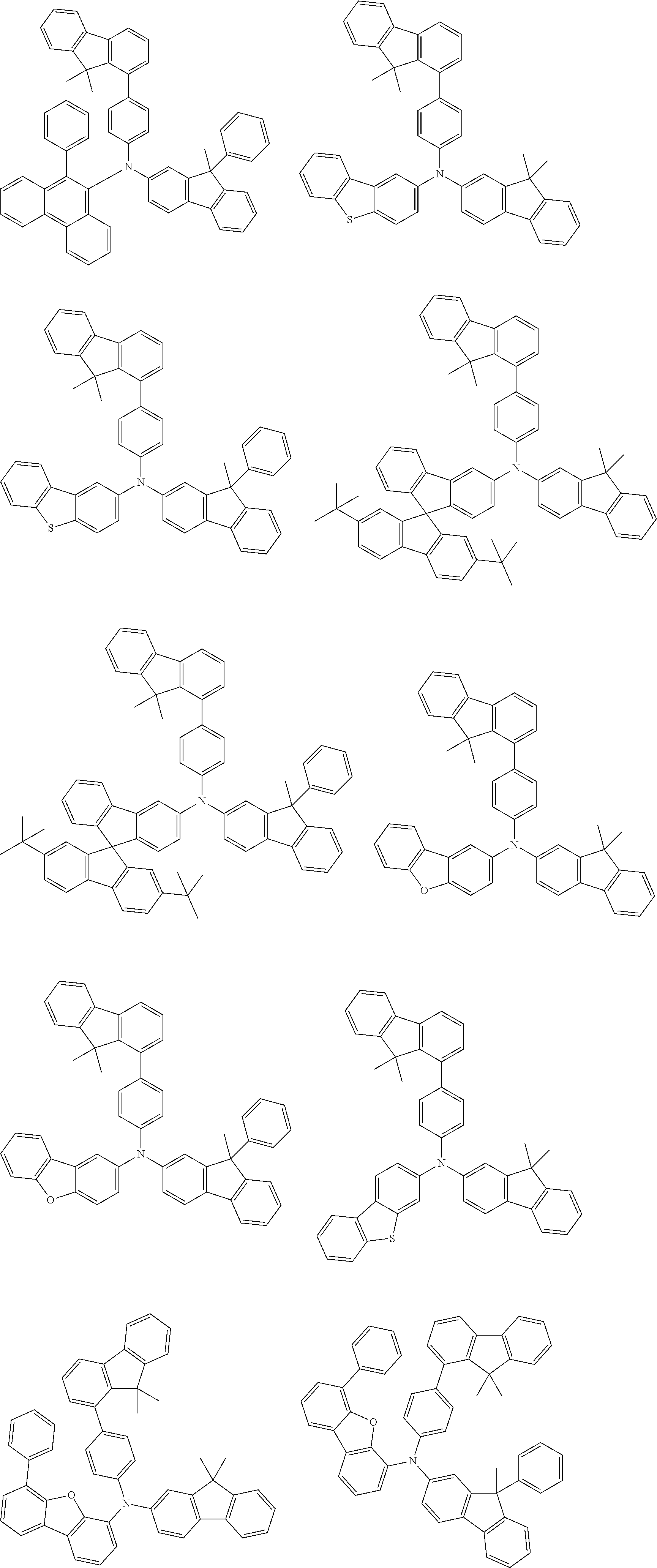

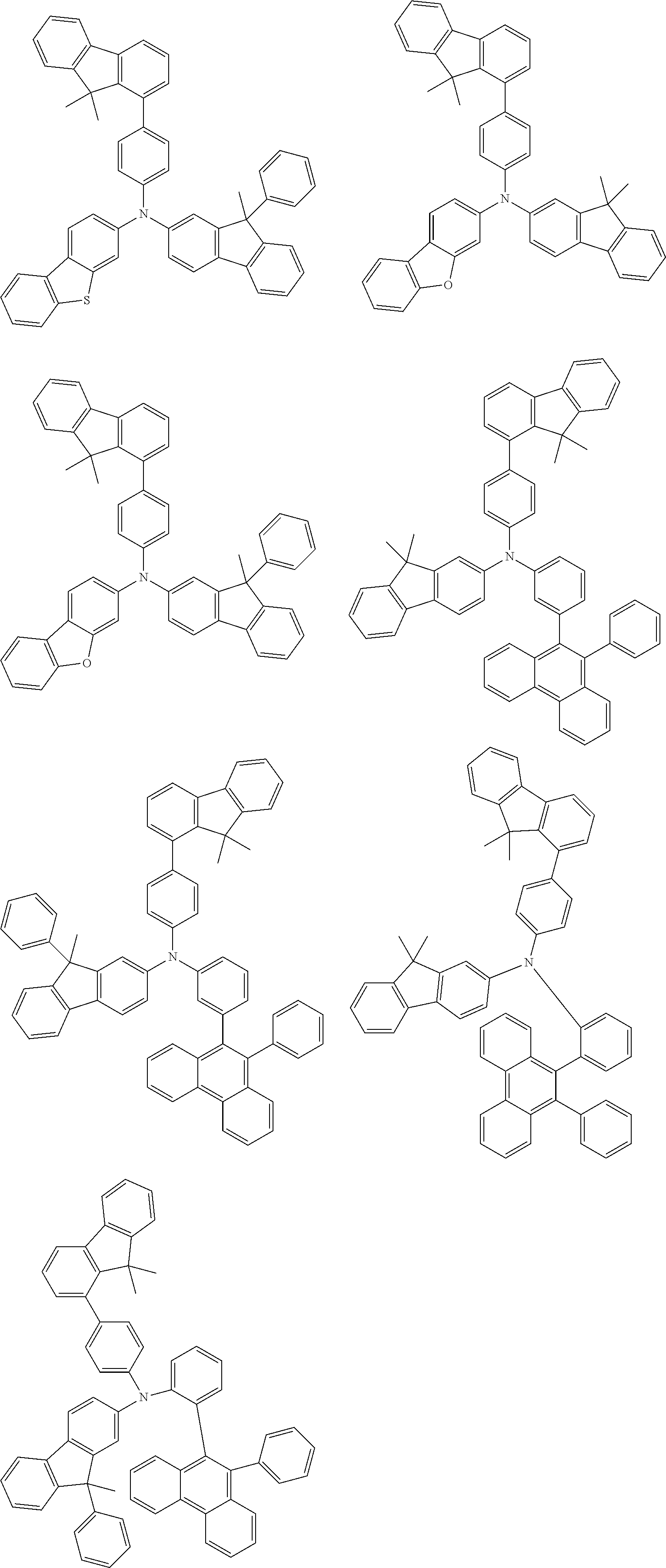

Material for Organic EL device

The material for an organic EL device of the present invention contains the inventive compound. The content of the inventive compound in the material for an organic EL device of the present invention may be 1% by mass or more (including 100%), and is preferably 10% by mass or more (including 100%), more preferably 50% by mass or more (including 100%), further preferably 80% by mass or more (including 100%), and particularly preferably 90% by mass or more (including 100%). The material for an organic EL device of the present invention is useful for the production of an organic EL device.

### Organic EL Device

The organic EL device of the present invention includes an anode, a cathode, and organic layers intervening between the anode and the cathode. The organic layers include a light emitting layer, and at least one layer of the organic layers contains the inventive compound.

Examples of the organic layer containing the inventive compound include a hole transporting zone (such as a hole injecting layer, a hole transporting layer, an electron blocking layer, and an exciton blocking layer) intervening between the anode and the light emitting layer, the light emitting layer, a space layer, and an electron transporting zone (such as an electron injecting layer, an electron transporting layer, and a hole blocking layer) intervening between the cathode and the light emitting layer, but are not limited thereto. The inventive compound is preferably used as a material for the hole transporting zone or the light emitting layer of a fluorescent or phosphorescent EL device, more preferably a material for the hole transporting zone thereof, further preferably a material for the hole injecting layer, the hole transporting layer, the electron blocking layer, or the exciton blocking layer thereof, and particularly preferably a material for the hole injecting layer or the hole transporting layer thereof.

The organic EL device of the present invention may be a fluorescent or phosphorescent light emission-type monochromatic light emitting element or a fluorescent/phosphorescent hybrid-type white light emitting element, and may be a simple type having a single light emitting unit or a tandem type having multiple light emitting units. Above all, the fluorescent light emission-type element is preferred. The “light emitting unit” referred to herein refers to a minimum unit that emits light through recombination of injected holes and electrons, which includes organic layers among which at least one layer is a light emitting layer.

For example, as a representative element configuration of the simple type organic EL device, the following element configuration may be exemplified.

### (1) Anode/Light Emitting Unit/Cathode

The light emitting unit may be a multilayer type having multiple phosphorescent light emitting layers or fluorescent light emitting layers. In this case, a space layer may intervene between the light emitting layers for the purpose of preventing excitons generated in the phosphorescent light emitting layer from diffusing into the fluorescent light emitting layer. Representative layer configurations of the simple type light emitting unit are described below. Layers in parentheses are optional.
(a) (hole injecting layer/) hole transporting layer/fluorescent light emitting layer/electron transporting layer (/electron injecting layer)
(b) (hole injecting layer/)hole transporting layer/first fluorescent light emitting layer/second fluorescent light emitting layer/electron transporting layer(/electron injecting layer)
(c) (hole injecting layer/)hole transporting layer/phosphorescent light emitting layer/space layer/fluorescent light emitting layer/electron transporting layer(/electron injecting layer)
(d) (hole injecting layer/)hole transporting layer/first phosphorescent light emitting layer/second phosphorescent light emitting layer/space layer/fluorescent light emitting layer/electron transporting layer(/electron injecting layer)
(e) (hole injecting layer/)hole transporting layer/phosphorescent light emitting layer/space layer/first fluorescent light emitting layer/second fluorescent light emitting layer/electron transporting layer(/electron injecting layer)
(f) (hole injecting layer/)hole transporting layer/electron blocking layer/fluorescent light emitting layer/electron transporting layer(/electron injecting layer)
(g) (hole injecting layer/)hole transporting layer/exciton blocking layer/fluorescent light emitting layer/electron transporting layer(/electron injecting layer)
(h) (hole injecting layer/)first hole transporting layer/second hole transporting layer/fluorescent light emitting layer/electron transporting layer(/electron injecting layer)
(i) (hole injecting layer/)first hole transporting layer/second hole transporting layer/fluorescent light emitting layer/first electron transporting layer/second electron transporting layer(/electron injecting layer)
(j) (hole injecting layer/)hole transporting layer/fluorescent light emitting layer/hole blocking layer/electron transporting layer(/electron injecting layer)
(k) (hole injecting layer/)hole transporting layer/fluorescent light emitting layer/exciton blocking layer/electron transporting layer(/electron injecting layer)
(l) (hole injecting layer)first hole transporting layer/second hole transporting layer/first fluorescent light emitting layer/second fluorescent light emitting layer/first electron transporting layer/second electron transporting layer(/electron injecting layer)
(m) (hole injecting layer/)first hole transporting layer/second hole transporting layer/third hole transporting layer/first fluorescent light emitting layer/second fluorescent light emitting layer/first electron transporting layer/second electron transporting layer(/electron injecting layer)
(n) (hole injecting layer/)first hole transporting layer/second hole transporting layer/third hole transporting layer/fluorescent light emitting layer/first electron transporting layer/second electron transporting layer(/electron injecting layer)

The phosphorescent and fluorescent light emitting layers may emit emission colors different from each other, respectively. Specifically, in the light emitting unit (f), a layer configuration, such as (hole injecting layer/) hole transporting layer/first phosphorescent light emitting layer (red light emission)/second phosphorescent light emitting layer (green light emission)/space layer/fluorescent light emitting layer (blue light emission)/electron transporting layer, may be exemplified.

An electron blocking layer may be properly provided between each light emitting layer and the hole transporting layer or the space layer. A hole blocking layer may be properly provided between each light emitting layer and the electron transporting layer. The employment of the electron blocking layer or the hole blocking layer allows to improve the emission efficiency by trapping electrons or holes within the light emitting layer and increasing the probability of charge recombination in the light emitting layer.

As a representative element configuration of the tandem type organic EL device, the following element configuration may be exemplified.

### (2) Anode/First Light Emitting Unit/Intermediate Layer/Second Light Emitting Unit/Cathode

For example, each of the first light emitting unit and the second light emitting unit may be independently selected from the above-described light emitting units.

The intermediate layer is also generally referred to as an intermediate electrode, an intermediate conductive layer, a charge generation layer, an electron withdrawing layer, a connecting layer, or an intermediate insulating layer, and a known material configuration can be used, in which electrons are supplied to the first light emitting unit, and holes are supplied to the second light emitting unit.

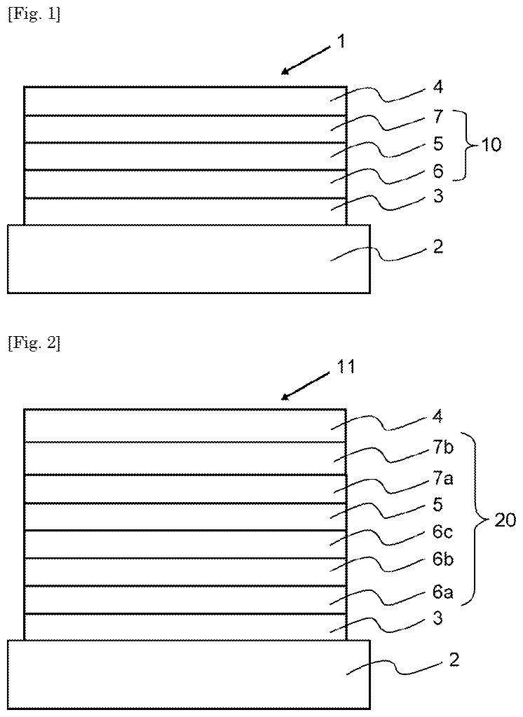

FIG. 1 is a schematic illustration showing an example of the configuration of the organic EL device of the present invention. An organic EL device 1 of this example includes a substrate 2, an anode 3, a cathode 4, and a light emitting unit 10 disposed between the anode 3 and the cathode 4. The light emitting unit 10 includes a light emitting layer 5. A hole transporting zone 6 (such as a hole injecting layer and a hole transporting layer) is provided between the light emitting layer 5 and the anode 3, and an electron transporting zone 7 (such as an electron injecting layer and an electron transporting layer) is provided between the light emitting layer 5 and the cathode 4. An electron blocking layer (which is not shown in the figure) may be provided on the side of the anode 3 of the light emitting layer 5, and a hole blocking layer (which is not shown in the figure) may be provided on the side of the cathode 4 of the light emitting layer 5. According to the configuration, electrons and holes are trapped in the light emitting layer 5, thereby enabling one to further increase the production efficiency of excitons in the light emitting layer 5.

FIG. 2 is a schematic illustration showing another configuration of the organic EL device of the present invention. An organic EL device 11 includes the substrate 2, the anode 3, the cathode 4, and a light emitting unit 20 disposed between the anode 3 and the cathode 4. The light emitting unit 20 includes the light emitting layer 5. A hole transporting zone disposed between the anode 3 and the light emitting layer 5 includes a hole injecting layer 6a, a first hole transporting layer 6b, and a second hole transporting layer 6c. An electron transporting zone disposed between the light emitting layer 5 and the cathode 4 includes a first electron transporting layer 7a and a second electron transporting layer 7b.

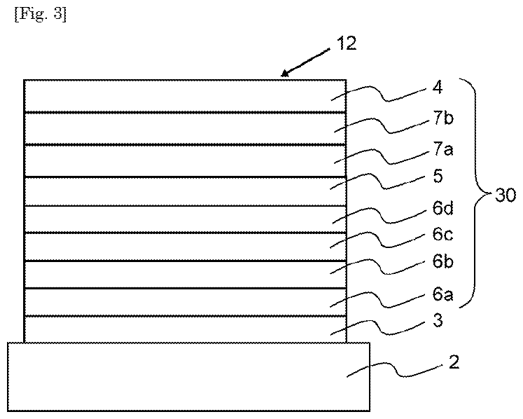

FIG. 3 is a schematic illustration showing still another configuration of the organic EL element of the present invention. An organic EL device 12 includes the substrate 2, the anode 3, the cathode 4, and a light emitting unit 30 disposed between the anode 3 and the cathode 4. The light emitting unit 30 includes the light emitting layer 5. A hole transporting zone disposed between the anode 3 and the light emitting layer 5 includes the hole injecting layer 6a, the first hole transporting layer 6b, the second hole transporting layer 6c, and a third hole transporting layer 6d. An electron transporting zone disposed between the light emitting layer 5 and the cathode 4 includes the first electron transporting layer 7a and the second electron transporting layer 7b.

In the present invention, a host combined with a fluorescent dopant material (a fluorescent emitting material) is referred to as a fluorescent host, and a host combined with a phosphorescent dopant material is referred to as a phosphorescent host. The fluorescent host and the phosphorescent host are not distinguished from each other merely by the molecular structures thereof. Specifically, the phosphorescent host means a material that forms a phosphorescent light emitting layer containing a phosphorescent dopant, but does not mean unavailability as a material that forms a fluorescent light emitting layer. The same also applies to the fluorescent host.

### Substrate

The substrate is used as a support of the organic EL device. Examples of the substrate include a plate of glass, quartz, and plastic. A flexible substrate may also be used. Examples of the flexible substrate include a plastic substrate made of polycarbonate, polyarylate, polyether sulfone, polypropylene, polyester, polyvinyl fluoride, or polyvinyl chloride. An inorganic vapor deposition film may also be used.

### Anode

It is preferred that a metal, an alloy, an electrically conductive compound, or a mixture thereof which has a high work function (specifically 4.0 eV or more) is used for the anode formed on the substrate. Specific examples thereof include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. Besides, examples thereof include gold (Au), platinum (Pt), nickel (Ni), tungsten (W), chromium (Cr), molybdenum (Mo), iron (Fe), cobalt (Co), copper (Cu), palladium (Pd), titanium (Ti), or nitrides of the metals (for example, titanium nitride).

These materials are usually deposited by a sputtering method. For example, through a sputtering method, it is possible to form indium oxide-zinc oxide by using a target in which 1 to 10 wt % of zinc oxide is added to indium oxide, and to form indium oxide containing tungsten oxide and zinc oxide by using a target containing 0.5 to 5 wt % of tungsten oxide and 0.1 to 1 wt % of zinc oxide with respect to indium oxide. Besides, the manufacturing may be performed by a vacuum vapor deposition method, a coating method, an inkjet method, a spin coating method, or the like.

### Hole Transporting Zone

As described above, the organic layers may include a hole transporting zone intervening between the anode and the light emitting layer. The hole transporting zone is constituted by a hole injecting layer, a hole transporting layer, an electron blocking layer, and the like. The hole transporting zone preferably contains the inventive compound. The inventive compound is preferably contained in at least one layer of these layers constituting the hole transporting zone, and the inventive compound is particularly preferably contained in the hole transporting layer.

The hole injecting layer formed in contact with the anode is formed by using a material that facilitates hole injection regardless of a work function of the anode, and thus, it is possible to use materials generally used as an electrode material (for example, metals, alloys, electrically conductive compounds, or mixtures thereof, elements belonging to Group 1 or 2 of the periodic table of the elements).

It is also possible to use elements belonging to Group 1 or 2 of the periodic table of the elements, which are materials having low work functions, that is, alkali metals, such as lithium (Li) and cesium (Cs), alkaline earth metals, such as magnesium (Mg), calcium (Ca), and strontium (Sr), and alloys containing these (such as MgAg and AlLi), and rare earth metals, such as europium (Eu), and ytterbium (Yb) and alloys containing these. In the case where the anode is formed by using the alkali metals, the alkaline earth metals, and alloys containing these, a vacuum vapor deposition method or a sputtering method can be used. In the case where a silver paste or the like is used, a coating method, an inkjet method, or the like can be used.

### Hole Injecting Layer

The hole injecting layer is a layer containing a material having a high hole injection capability (a hole injecting material) and is provided between the anode and the light emitting layer, or between the hole transporting layer, if exists, and the anode.

Examples of the hole injecting material other than the inventive compound include molybdenum oxide, titanium oxide, vanadium oxide, rhenium oxide, ruthenium oxide, chromium oxide, zirconium oxide, hafnium oxide, tantalum oxide, silver oxide, tungsten oxide, and manganese oxide.

Examples of the hole injecting layer material also include aromatic amine compounds as low-molecular weight organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

High-molecular weight compounds (such as oligomers, dendrimers, and polymers) may also be used. Examples thereof include high-molecular weight compounds, such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). High-molecular weight compounds to which an acid is added, such as poly(3,4-ethylenedioxythiophene)/poly (styrene sulfonic acid) (PEDOT/PSS), and polyaniline/poly (styrenesulfonic acid) (PAni/PSS), can also be used.

It is also preferred to use an acceptor material, such as a hexaazatriphenylene (HAT) compound represented by the following formula (K).

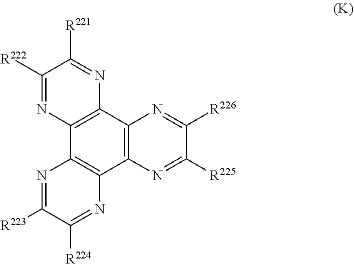

In the aforementioned formula, R²²¹ to R²²⁶ each independently represent a cyano group, —CONH₂, a carboxy group, or —COOR²²⁷ (wherein R²²⁷ represents an alkyl group having 1 to 20 carbon atoms or a cycloalkyl group having 3 to 20 carbon atoms). Adjacent two selected from R²²¹ and R²²², R²²³ and R²²⁴, and R²²⁵ and R²²⁶ may be bonded to each other to form a group represented by —CO—O—CO—.

Examples of R²²⁷ include a methyl group, an ethyl group, a n-propyl group, an isopropyl group, a n-butyl group, an isobutyl group, a t-butyl group, a cyclopentyl group, and a cyclohexyl group.

### Hole Transporting Layer

The hole transporting layer is a layer containing a material having a high hole transporting capability (a hole transporting material) and is provided between the anode and the light emitting layer, or between the hole injecting layer, if exists, and the light emitting layer. The inventive compound may be used alone or as a combination with the following compounds in the hole transporting layer.

The hole transporting layer may have a single layer structure or a multilayer structure including two or more layers. For example, the hole transporting layer may have a two-layer structure including a first hole transporting layer (anode side) and a second hole transporting layer (cathode side). Specifically, the hole transporting zone may include the first hole transporting layer on the anode side and the second hole transporting layer on the cathode side. The hole transporting layer may have a three-layer structure including a first hole transporting layer, a second hole transporting layer, and a third hole transporting layer in this order from the anode side. Specifically, the third hole transporting layer may be disposed between the second hole transporting layer and the light emitting layer.

In one embodiment of the present invention, the hole transporting layer having a single layer structure is preferably disposed adjacent to the light emitting layer, and the hole transporting layer that is closest to the cathode in the multilayer structure, such as the second hole transporting layer in the two-layer structure and the third hole transporting layer in the three-layer structure, is preferably disposed adjacent to the light emitting layer. In another embodiment of the present invention, an electron blocking layer described later and the like may be disposed between the hole transporting layer having a single layer structure and the light emitting layer, or between the hole transporting layer that is closest to the light emitting layer in the multilayer structure and the light emitting layer.

In the case where the hole transporting layer has the two-layer structure, at least one of the first hole transporting layer and the second hole transporting layer contains the inventive compound. Specifically, the inventive compound is contained only in the first hole transporting layer, only in the second hole transporting layer, or in both the first hole transporting layer and the second hole transporting layer.

In one embodiment of the present invention, the inventive compound is preferably contained in the second hole transporting layer. Specifically, it is preferred that the inventive compound is contained only in the second hole transporting layer, or the inventive compound is contained in in both the first hole transporting layer and the second hole transporting layer.

In the case where the hole transporting layer has the three-layer structure, at least one of the first to third hole transporting layers contains the inventive compound. Specifically, the inventive compound is contained only in one layer selected from the first to third hole transporting layers (i.e., only in the first hole transporting layer, only in the second hole transporting layer, or only in the third hole transporting layer), only in two layers selected from the first to third hole transporting layers (i.e., only in the first hole transporting layer and the second hole transporting layer, only in the first hole transporting layer and the third hole transporting layer, or only in the second hole transporting layer and the third hole transporting layer), or in all the first to third hole transporting layers.

In one embodiment of the present invention, the inventive compound is preferably contained in the third hole transporting layer. Specifically, it is preferred that the inventive compound is contained only in the third hole transporting layer, or the inventive compound is contained in the third hole transporting layer and one or both of the first hole transporting layer and the second hole transporting layer.

In one embodiment of the present invention, the inventive compound contained in the hole transporting layers is preferably a protium compound from the standpoint of the production cost. The protium compound herein means an inventive compound having all the hydrogen atoms in the inventive compound that are protium atoms.

Accordingly, the present invention includes an organic EL device having one or both of the first hole transporting layer and the second hole transporting layer (in the two-layer structure), or at least one of the first to third hole transporting layers that contains the inventive compound formed substantially only of the protium compound. The “inventive compound formed substantially only of the protium compound” means that the content ratio of the protium compound with respect to the total amount of the inventive compound is 90% by mol or more, preferably 95% by mol or more, and more preferably 99% by mol or more (each of which includes 100%).

As a material for the hole transporting layer other than the inventive compound, for example, an aromatic amine compound, a carbazole derivative, an anthracene derivative, and the like can be used.

Examples of the aromatic amine compound include 4,4′-bis[N-(1-naphthyl)-N-phenylamino]biphenyl (abbreviation: NPB) or N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The aforementioned compounds have a hole mobility of 106 cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

High-molecular weight compounds, such as poly(N-vinylcarbazole) (abbreviation: PVK) and poly(4-vinyltriphenylamine) (abbreviation: PVTPA), can also be used.

However, compounds other than those as mentioned above can also be used so long as they are compounds high in the hole transporting capability rather than in the electron transporting capability.

In the organic EL device having a hole transporting layer having the two-layer structure, the first hole transporting layer preferably contains one kind or multiple kinds of a compound represented by the following formula (11) or (12).

In the organic EL device having a hole transporting layer having the three-layer structure, one or both of the first hole transporting layer and the second hole transporting layer preferably contains one kind or multiple kinds of a compound represented by the following formula (11) or (12).

In the organic EL device having a hole transporting layer having an n-layer structure (wherein n represents an integer of 4 or more), at least one layer of the first hole transporting layer to the (n−1)th hole transporting layer preferably contains one kind or multiple kinds of a compound represented by the following formula (11) or (12).

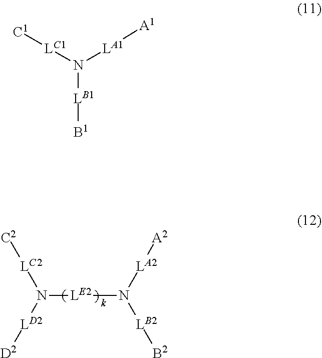

wherein in the formulae (11) and (12),
L^{A1}, L^{B1}, L^{C1}, L^{A2}, L^{B2}, L^{C2}, and L^{D2} each independently represent a single bond, a substituted or unsubstituted arylene group having 6 to 50 ring carbon atoms, or a substituted or unsubstituted divalent heterocyclic group having 5 to 50 ring atoms,
k represents 1, 2, 3, or 4,
in the case where k represents 1, L^{E2} represents a substituted or unsubstituted arylene group having 6 to 50 ring carbon atoms or a substituted or unsubstituted divalent heterocyclic group having 5 to 50 ring atoms,
in the case where k represents 2, 3, or 4, 2, 3, or 4 groups represented by L^{E2} may be the same as or different from each other,
in the case where k represents 2, 3, or 4, multiple groups represented by L^{E2} may be bonded to each other to form a substituted or unsubstituted monocyclic ring, may be bonded to each other to form a substituted or unsubstituted condensed ring, or may not bonded to each other,
L^{E2} that does not form a monocyclic ring and does not form a condensed ring represents a substituted or unsubstituted arylene group having 6 to 50 ring carbon atoms, or a substituted or unsubstituted divalent heterocyclic group having 5 to 50 ring atoms,
A¹, B¹, C¹, A², B², C², and D² each independently represent a substituted or unsubstituted aryl group having 6 to 50 ring carbon atoms, a substituted or unsubstituted heterocyclic group having 5 to 50 ring atoms, or —Si(R′₉₀₁)(R′₉₀₂)(R′₉₀₃),
R′₉₀₁, R′₉₀₂, and R′₉₀₃ each independently represent a substituted or unsubstituted aryl group having 6 to 50 ring carbon atoms,
in the case where multiple groups represented by R′₉₀₁ exist, the multiple groups represented by R′₉₀₁ may be the same as or different from each other,
in the case where multiple groups represented by R′₉₀₂ exist, the multiple groups represented by R′₉₀₂ may be the same as or different from each other, and
in the case where multiple groups represented by R′₉₀₃ exist, the multiple groups represented by R′₉₀₃ may be the same as or different from each other.

In the formulae (11) and (12), it is preferred that A¹, B¹, C¹, A², B², C², and D² each are independently selected from a substituted or unsubstituted phenyl group, a substituted or unsubstituted biphenylyl group, a substituted or unsubstituted terphenylyl group, a substituted or unsubstituted naphthyl group, a substituted or unsubstituted fluorenyl group, a substituted or unsubstituted dibenzofuranyl group, a substituted or unsubstituted dibenzothiophenyl group, and a substituted or unsubstituted carbazolyl group.

It is more preferred that at least one of A¹, B¹ and C¹ in the formula (11) and at least one of A², B², C², and D² in the formula (12) represents a substituted or unsubstituted biphenylyl group, a substituted or unsubstituted terphenylyl group, a substituted or unsubstituted naphthyl group, a substituted or unsubstituted fluorenyl group, a substituted or unsubstituted dibenzofuranyl group, a substituted or unsubstituted dibenzothiophenyl group, or a substituted or unsubstituted carbazoyl group.

The fluorenyl group that can be represented by A¹, B¹, C¹, A², B², C², and D² may have a substituent at the 9-position thereof, and may be, for example, a 9,9-dimethylfluorenyl group or a 9,9-diphenylfluorenyl group. The substituents at the 9-position may form a ring, and for example, the substituents at the 9-position may form a fluorene skeleton or a xanthene skeleton.

L^{A1}, L^{B1}, L^{C1}, L^{A2}, L^{B2}, L^{C2}, and L^{D2} each independently preferably represent a single bond or a substituted or unsubstituted arylene group having 6 to 12 ring carbon atoms.

Specific examples of the compound represented by the formulae (11) and (12) include the following compounds.

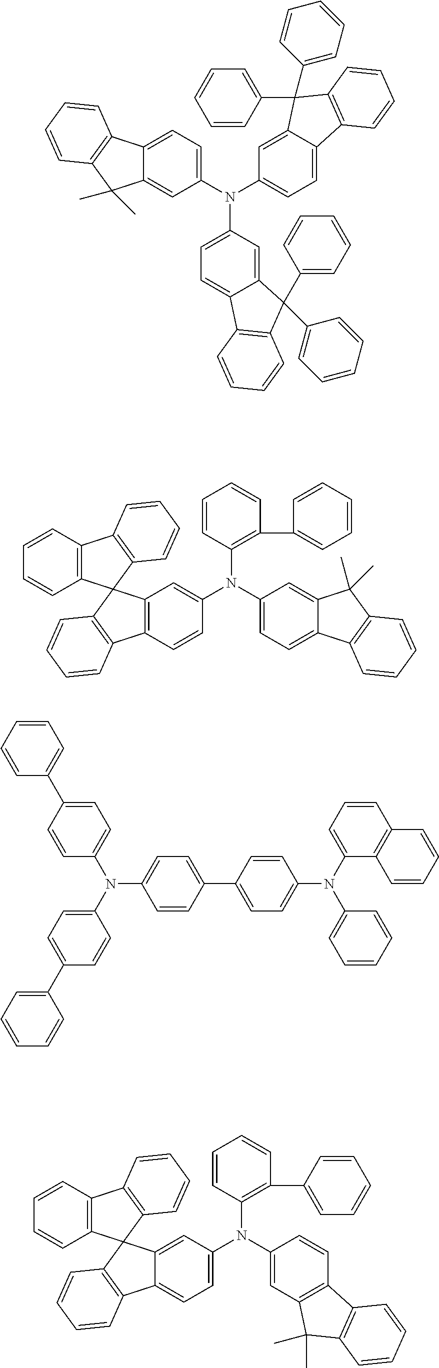 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc4c(c3)C3(c5ccccc5-c5ccccc53)c3ccccc3-4)c3ccccc3-c3ccccc3)cc21]

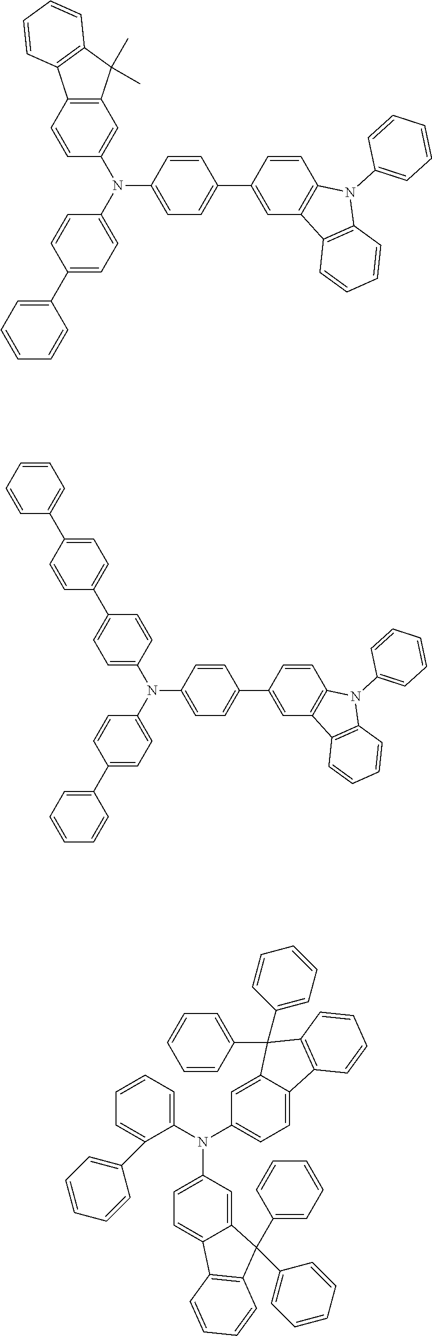 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

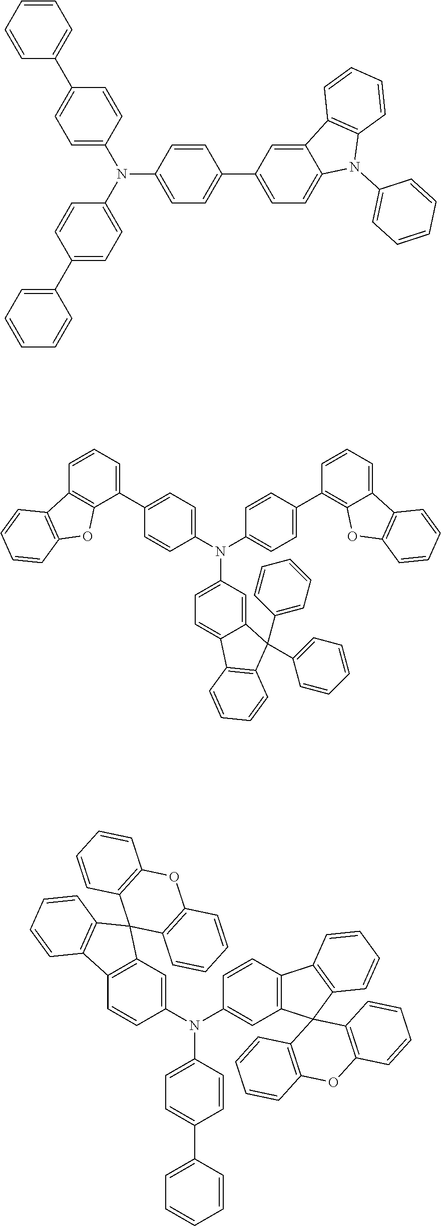 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1]

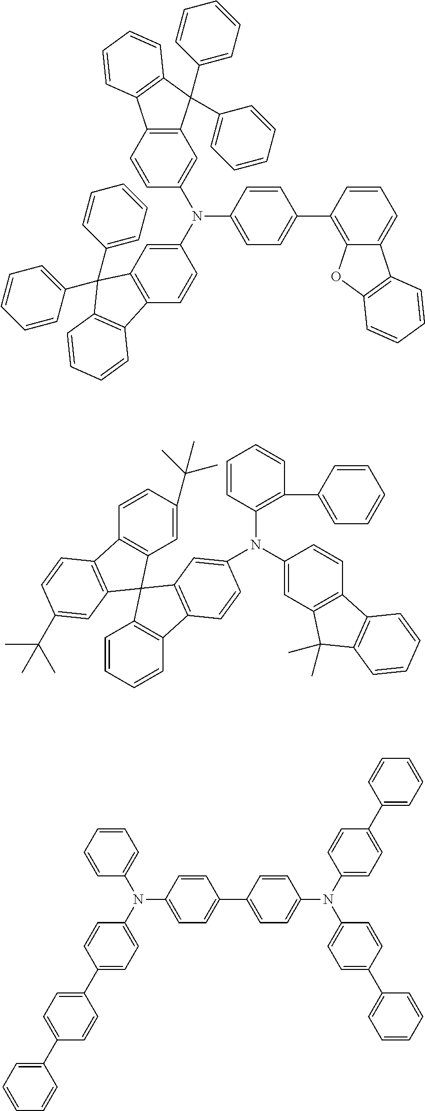 [SMILES: c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

### Dopant Material of Light Emitting Layer

The light emitting layer is a layer containing a material having a high light emitting property (a dopant material), and various materials can be used. For example, a fluorescent emitting material or a phosphorescent emitting material can be used as the dopant material. The fluorescent emitting material is a compound that emits light from a singlet excited state, and the phosphorescent emitting material is a compound that emits from a light triplet excited state.

In one embodiment of the organic EL device according to the present invention, the light emitting layer is a single layer.

In another embodiment of the organic EL device according to the present invention, the light emitting layer includes a first light emitting layer and a second light emitting layer.

Examples of a blue-based fluorescent emitting material that can be used for the light emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a green-based fluorescent emitting material that can be used for the light emitting layer include an aromatic amine derivative. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), and N,N,9-triphenylanthracene-9-amine (abbreviation: DPhAPhA).

Examples of a red-based fluorescent emitting material that can be used for the light emitting layer include a tetracene derivative and a diamine derivative. Specific examples thereof include N,N,N′,N′-tetrakis(4-methylphenyl)tetracene-5,11-diamine (abbreviation: p-mPhTD) and 7,14-diphenyl-N,N,N′,N′-tetrakis(4-methylphenyl)acenaphtho[1,2-a]fluoranthene-3,10-diamine (abbreviation: p-mPhAFD).

In one embodiment of the present invention, the light emitting layer preferably contains a fluorescent light emitting material (a fluorescent dopant material).

Examples of a blue-based phosphorescent emitting material that can be used for the light emitting layer include a metal complex, such as an iridium complex, an osmium complex, and a platinum complex. Specific examples thereof include bis[2-(4′,6′-difluorophenyl)pyridinato-N,C2′]iridium(III)tetrakis(1-pyrazolyl)borate (abbreviation: FIr6), bis[2-(4′,6′-difluorophenyl)pyridinato-N,C2′]iridium(III)picolinate (abbreviation: FIrpic), bis[2-(3′,5′bistrifluoromethylphenyl)pyridinato-N,C2′]iridium(III)picolinate (abbreviation: Ir(CF3ppy)2(pic)), and bis[2-(4′,6′-difluorophenyl)pyridinato-N,C2′]iridium(III)acetylacetonate (abbreviation: FIracac).

Examples of a green-based phosphorescent emitting material that can be used for the light emitting layer include an iridium complex. Examples thereof include tris(2-phenylpyridinato-N,C2′)iridium(III) (abbreviation: Ir(ppy)3), bis(2-phenylpyridinato-N,C2′)iridium(III)acetylacetonate (abbreviation: Ir(ppy)2(acac)), bis(1,2-diphenyl-1H-benzimidazolato)iridium(III)acetylacetonate (abbreviation: Ir(pbi)2(acac)), and bis(benzo[h]quinolinato)iridium(III)acetylacetonate (abbreviation: Ir(bzq)2(acac)).

Examples of a red-based phosphorescent emitting material that can be used for the light emitting layer include a metal complex, such as an iridium complex, a platinum complex, a terbium complex, and a europium complex. Specific examples thereof include organic metal complexes, such as bis[2-(2′-benzo[4,5-a]thienyl)pyridinato-N,C3′]iridium(III)acetylacetonate (abbreviation: Ir(btp)2(acac)), bis(1-phenylisoquinolinato-N,C2′)iridium(III)acetylacetonate (abbreviation: Ir(piq)2(acac)), (acetylacetonate)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrinplatinum(II) (abbreviation: PtOEP).

Rare earth metal complexes, such as tris(acetylacetonate) (monophenanthroline)terbium(II) (abbreviation: Tb(acac)3(Phen)), tris(1,3-diphenyl-1,3-propanedionato)(monophenanthroline)europium(III) (abbreviation: Eu(DBM)3(Phen)), and tris[1-(2-thenoyl)-3,3,3-trifluoroacetonate](monophenanthroline)europium(III) (abbreviation: Eu(TTA)3(Phen)), emit light from rare earth metal ions (electron transition between different multiplicities), and thus may be used as the phosphorescent emitting material.

### Host Material of Light Emitting Layer

The light emitting layer may have a configuration in which the aforementioned dopant material is dispersed in another material (a host material). The host material is preferably a material that has a higher lowest unoccupied molecular orbital level (LUMO level) and a lower highest occupied molecular orbital level (HOMO level) than the dopant material.

Examples of the host material include:
(1) a metal complex, such as an aluminum complex, a beryllium complex, and a zinc complex,
(2) a heterocyclic compound, such as an oxadiazole derivative, a benzimidazole derivative, and a phenanthroline derivative,
(3) a fused aromatic compound, such as a carbazole derivative, an anthracene derivative, a phenanthrene derivative, a pyrene derivative, and a chrysene derivative, or
(4) an aromatic amine compound, such as a triarylamine derivative and a fused polycyclic aromatic amine derivative.

For example,
metal complexes, such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(II) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), and bathophenanthroline (abbreviation: BPhen), bathocuproine (abbreviation: BCP);
fused aromatic compounds, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and
aromatic amine compounds, such as N,N-diphenyl-9-[4-(10-phenyl-9-anthryl)phenyl]-9H-carbazole-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N,9-diphenyl-N-{4-[4-(10-phenyl-9-anthryl)phenyl]phenyl}-9H-carbazole-3-amine (abbreviation: PCAPBA), N-(9,10-diphenyl-2-anthryl)-N,9-diphenyl-9H-carbazole-3-amine (abbreviation: 2PCAPA), 4,4′-bis[N-(1-naphthyl)-N-phenylamino]biphenyl (abbreviation: NPB or a-NPD), N,N′-bis(3-methylphenyl)-N,N′-diphenyl-[1,1′-biphenyl]-4,4′-diamine (abbreviation: TPD), 4,4′-bis[N-(9,9-dimethylfluorene-2-yl)-N-phenylamino]biphenyl (abbreviation: DFLDPBi), and 4,4′-bis[N-(spiro-9,9′-bifluorene-2-yl)-N-phenylamino]biphenyl (abbreviation: BSPB) can be used. Multiple kinds of the host materials may be used.

In particular, in the case of a blue fluorescent device, it is preferred to use the following anthracene compounds as the host material.

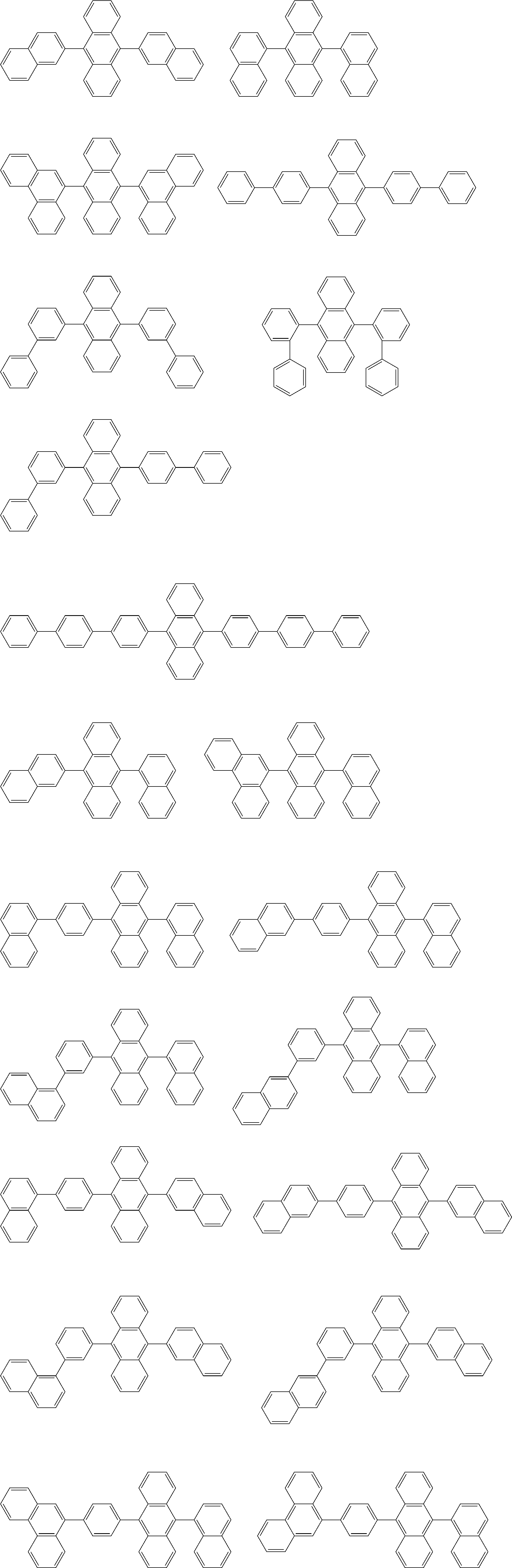

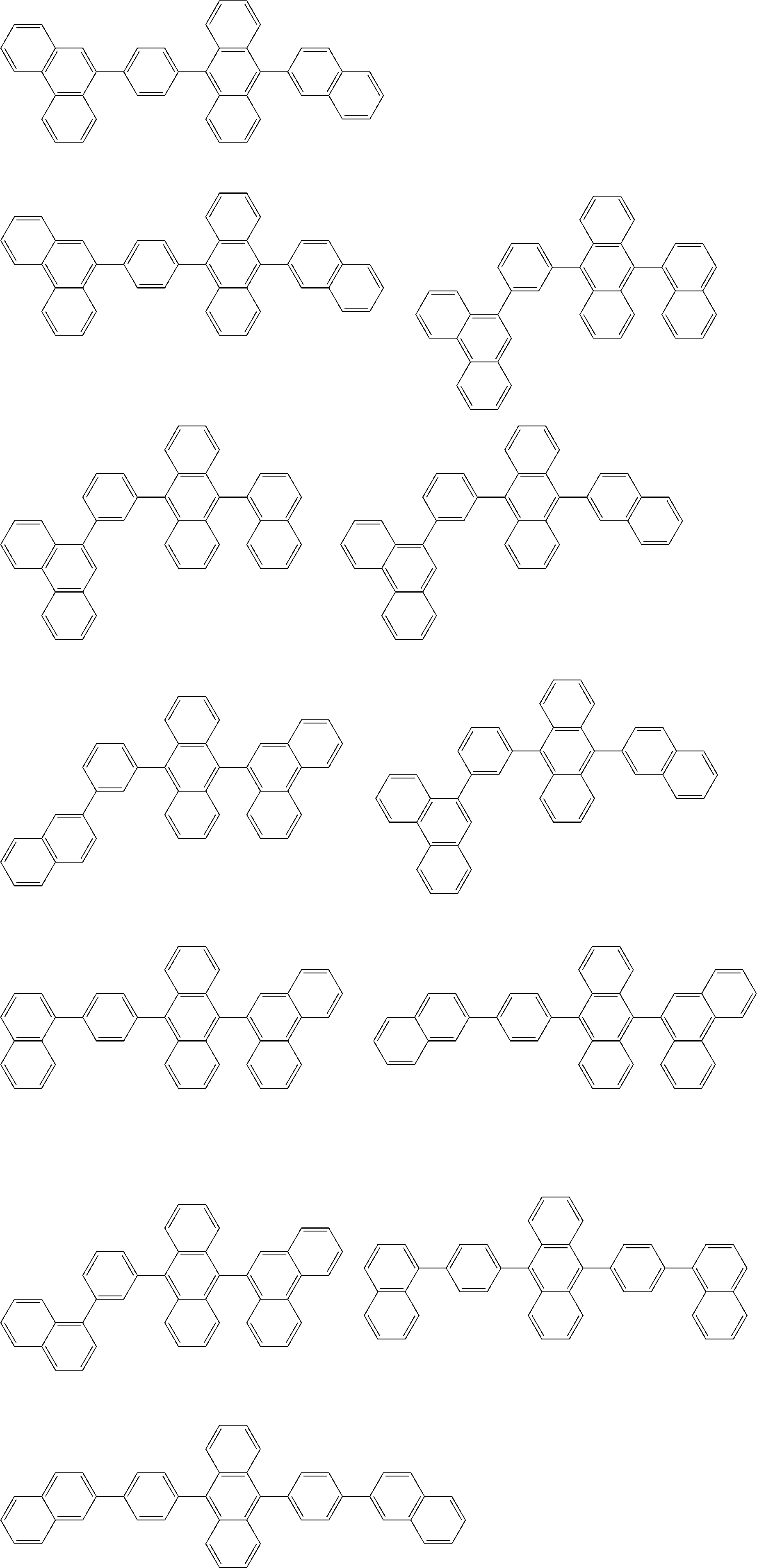

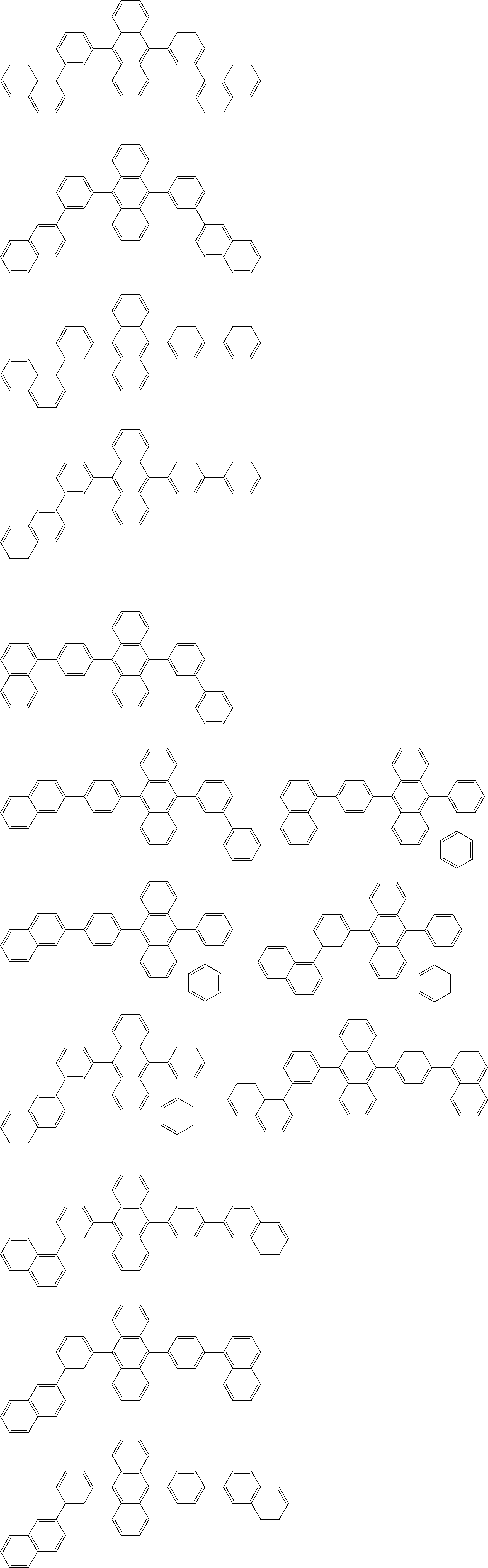

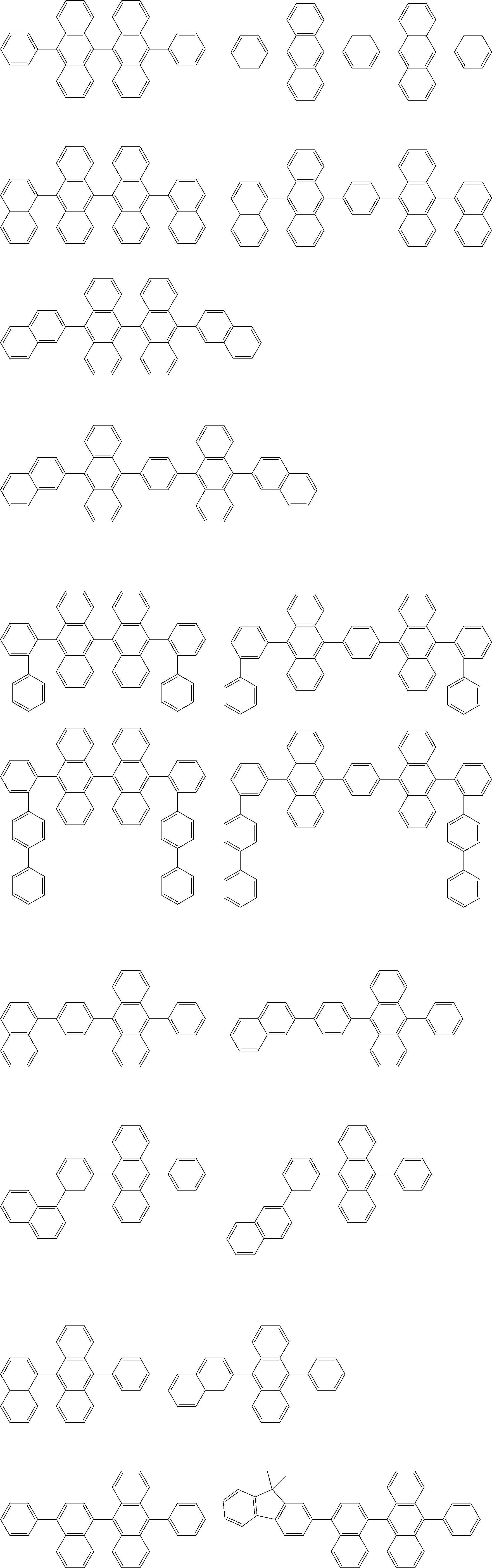

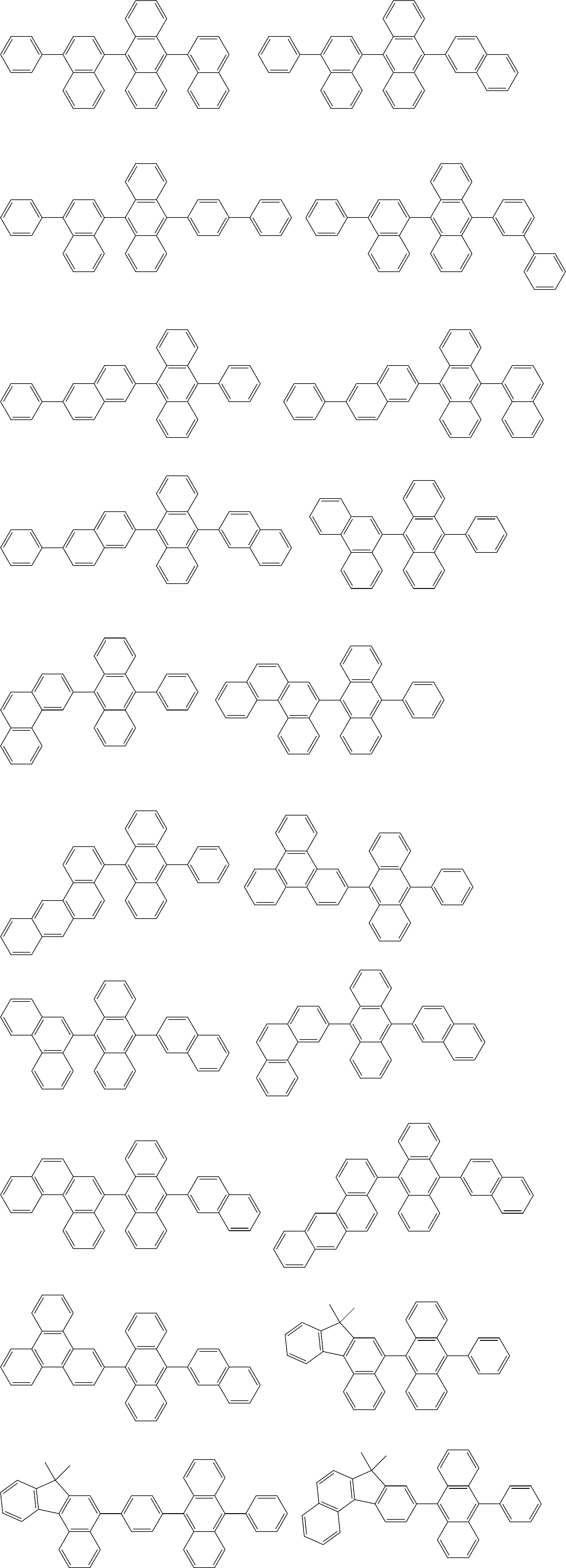

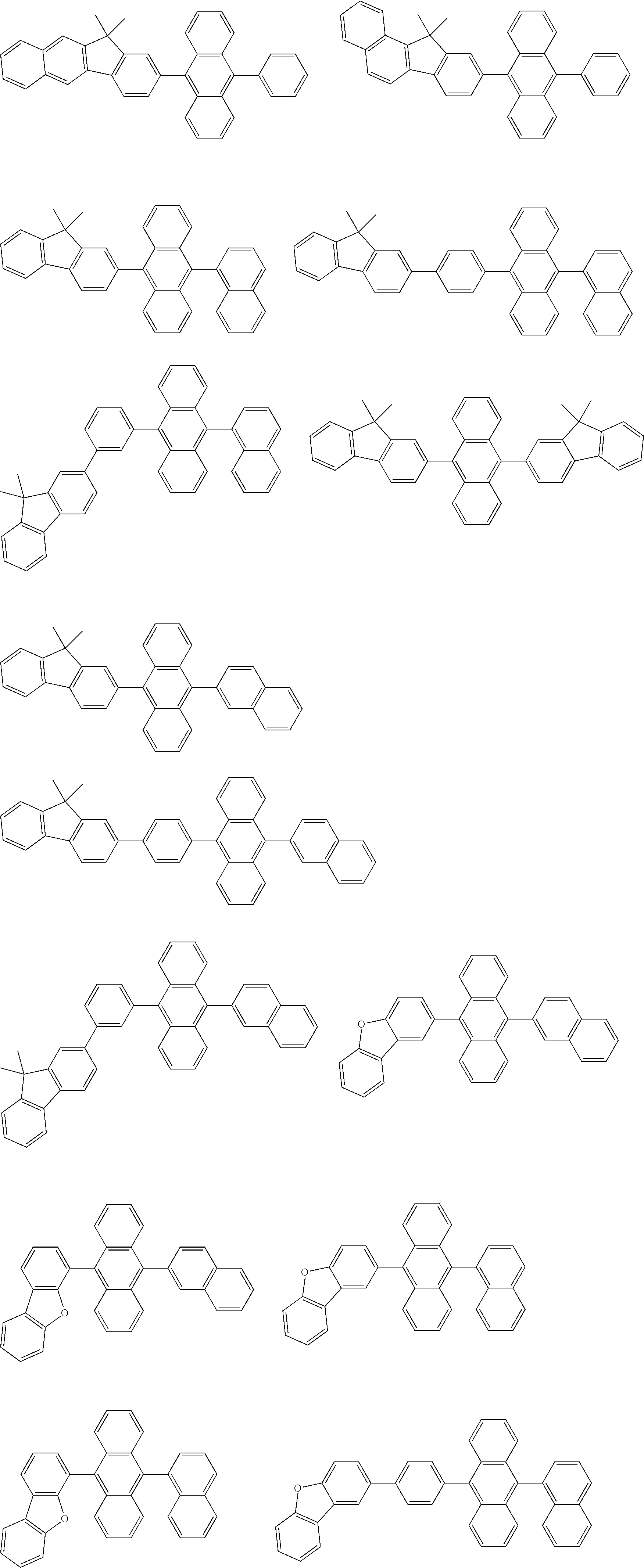

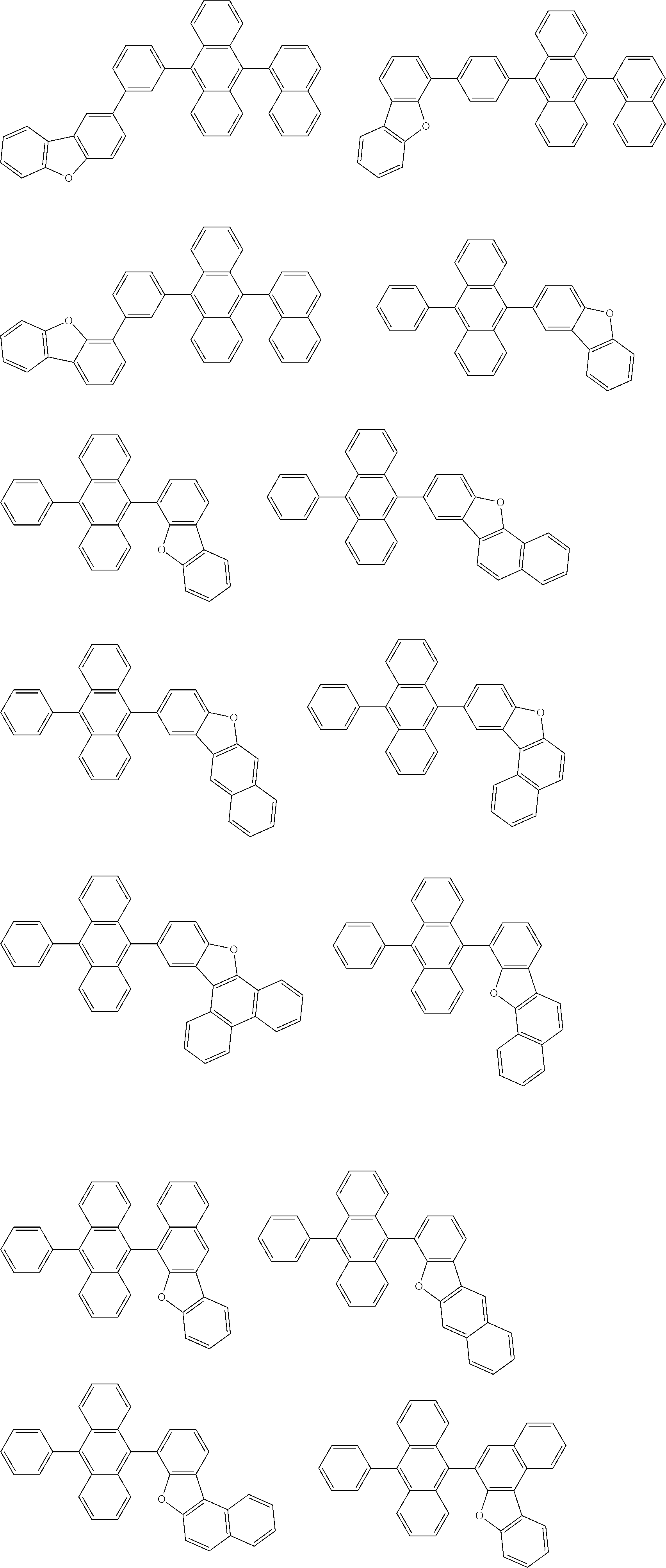

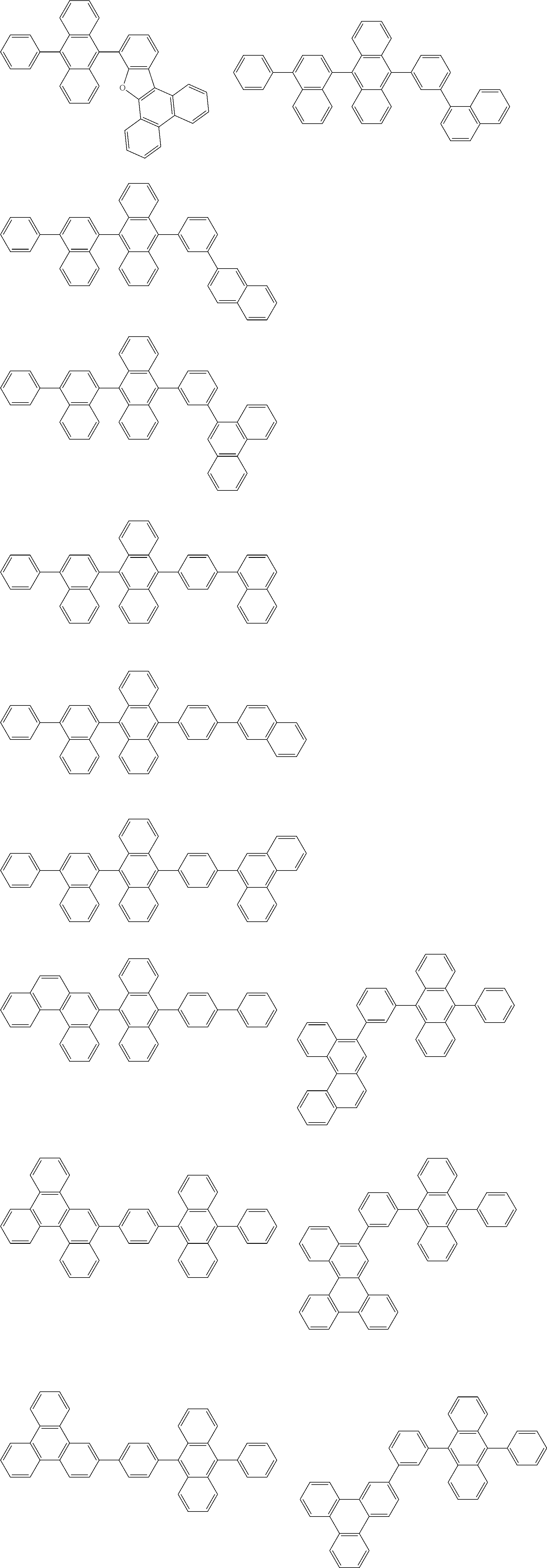

In one embodiment of the organic EL device according to the present invention, in the case where the light emitting layer includes a first light emitting layer and a second light emitting layer, at least one of the components constituting the first light emitting layer is different from the components constituting the second light emitting layer. Examples thereof include an embodiment in which the dopant material contained in the first light emitting layer is different from the dopant material contained in the second light emitting layer, and an embodiment in which the host material contained in the first light emitting layer is different from the host material contained in the second light emitting layer.

In the organic EL device of the present invention, the light emitting layer may contain a light emitting compound exhibiting fluorescent light emission having a main peak wavelength of 500 nm or less (which may be hereinafter referred simply to as a “fluorescent light emitting compound”).

The measurement method of the main peak wavelength of the compound is as follows. A 5 μmol/L toluene solution of the compound to be measured is prepared and placed in a quartz cell, and the light emission spectrum (ordinate: light emission intensity, abscissa: wavelength) of the specimen is measured at ordinary temperature (300 K). The light emission spectrum may be measured with a spectrofluorometer, available from Hitachi High-Tech Science Corporation (equipment name: F-7000). The light emission spectrum measurement equipment is not limited to the equipment used herein.

In the light emission spectrum, the peak wavelength of the light emission spectrum at which the light emission intensity is maximum is referred to as a main peak wavelength. In the description herein, the main peak wavelength may be referred to as a fluorescent light emission main peak wavelength (FL-peak).

The fluorescent light emitting compound may be the dopant material described above, or may be the host material described above.

In the case where the light emitting layer is a single layer, only one of the dopant material and the host material may be the fluorescent light emitting compound, or both of them may be the fluorescent light emitting compound.

In the case where the light emitting layer includes a first light emitting layer (anode side) and a second light emitting layer (cathode side), only one of the first light emitting layer and the second light emitting layer may contain the fluorescent light emitting compound, or both the light emitting layers may contain the fluorescent light emitting compound. In the case where the first light emitting layer contains the fluorescent light emitting compound, only one of the dopant material and the host material contained in the first light emitting layer may be the fluorescent light emitting compound, or both of them may be the fluorescent light emitting compound. In the case where the second light emitting layer contains the fluorescent light emitting compound, only one of the dopant material and the host material contained in the second light emitting layer may be the fluorescent light emitting compound, or both of them may be the fluorescent light emitting compound.

### Electron Transporting Layer

The electron transporting layer is a layer containing a material having a high electron transporting capability (an electron transporting material) and is provided between the light emitting layer and the cathode, or between the electron injecting layer, if exists, and the light emitting layer.

The electron transporting layer may have a single layer structure or a multilayer structure including two or more layers. For example, the electron transporting layer may have a two-layer structure including a first electron transporting layer (anode side) and a second electron transporting layer (cathode side). In one embodiment of the present invention, the electron transporting layer having a single layer structure is preferably disposed adjacent to the light emitting layer, and the electron transporting layer that is closest to the anode in the multilayer structure, such as the first electron transporting layer in the two-layer structure, is preferably disposed adjacent to the light emitting layer. In another embodiment of the present invention, a hole blocking layer described later and the like may be disposed between the electron transporting layer having a single layer structure and the light emitting layer, or between the electron transporting layer that is closest to the light emitting layer in the multilayer structure and the light emitting layer.

Examples of the material used in the electron transporting layer include:
(1) a metal complex, such as an aluminum complex, a beryllium complex, and a zinc complex;
(2) a heteroaromatic compound, such as an imidazole derivative, a benzimidazole derivative, an azine derivative, a carbazole derivative, and a phenanthroline derivative; and
(3) a high-molecular weight compound.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazol-2-yl)stilbene (abbreviation: BzOs).

Examples of the high-molecular weight compound include poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy).

The materials are materials having an electron mobility of 10⁻⁸ cm²/Vs or more. Materials other than those as mentioned above may also be used in the electron transporting layer so long as they are materials high in the electron transporting capability rather than in the hole transporting capability.

### Electron Injecting Layer

The electron injecting layer is a layer containing a material having a high electron injection capability. In the electron injecting layer, alkali metals, such as lithium (Li) and cesium (Cs), alkaline earth metals, such as magnesium (Mg), calcium (Ca), and strontium (Sr), rare earth metals, such as europium (Eu) and ytterbium (Yb), and compounds containing these metals can be used. Examples of the compounds include an alkali metal oxide, an alkali metal halide, an alkali metal-containing organic complex, an alkaline earth metal oxide, an alkaline earth metal halide, an alkaline earth metal-containing organic complex, a rare earth metal oxide, a rare earth metal halide, and a rare earth metal-containing organic complex. These compounds may be used as a mixture of a plurality thereof.

In addition, a material having an electron transporting capability, in which an alkali metal, an alkaline earth metal, or a compound thereof is contained, specifically Alq in which magnesium (Mg) is contained may be used. In this case, electron injection from the cathode can be more efficiently performed.

Otherwise, in the electron injecting layer, a composite material obtained by mixing an organic compound with an electron donor may be used. Such a composite material is excellent in the electron injection capability and the electron transporting capability because the organic compound receives electrons from the electron donor. In this case, the organic compound is preferably a material excellent in transporting received electrons, and specifically, examples thereof include a material constituting the aforementioned electron transporting layer (such as a metal complex and a heteroaromatic compound). As the electron donor, a material having an electron donation property for the organic compound may be used. Specifically, alkali metals, alkaline earth metals, and rare earth metals are preferred, and examples thereof include lithium, cesium, magnesium, calcium, erbium, and ytterbium. An alkali metal oxide or an alkaline earth metal oxide is preferred, and examples thereof include lithium oxide, calcium oxide, and barium oxide. A Lewis base, such as magnesium oxide, can also be used. An organic compound, such as tetrathiafulvalene (abbreviation: TTF), can also be used.

### Cathode

It is preferred that a metal, an alloy, an electrically conductive compound, or a mixture thereof which has a low work function (specifically 3.8 eV or less) is used for the cathode. Specific examples of such a cathode material include devices belonging to group 1 or 2 of the periodic table of the elements, that is, alkali metals, such as lithium (Li) and cesium (Cs), alkaline earth metals, such as magnesium (Mg), calcium (Ca), and strontium (Sr), and alloys containing these (such as MgAg, and AlLi), and rare earth metals, such as europium (Eu), and ytterbium (Yb) and alloys containing these.

In the case where the cathode is formed by using the alkali metals, the alkaline earth metals, and the alloys containing these, a vacuum vapor deposition method or a sputtering method can be adopted. In the case where a silver paste or the like is used, a coating method, an inkjet method, of the like can be adopted.

By providing the electron injecting layer, the cathode can be formed using various conductive materials, such as Al, Ag, ITO, graphene, and indium oxide-tin oxide containing silicon or silicon oxide regardless of the magnitude of a work function. Such a conductive material can be deposited by using a sputtering method, an inkjet method, a spin coating method, or the like.

### Insulating Layer

The organic EL device applies an electric field to an ultrathin film, and thus, pixel defects are likely to occur due to leaks or short-circuiting. In order to prevent this, an insulating layer formed of an insulating thin film layer may be inserted between a pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate of these may also be used.

### Space Layer

The space layer is, for example, a layer provided between a fluorescent light emitting layer and a phosphorescent light emitting layer for the purpose of preventing excitons generated in the phosphorescent light emitting layer from diffusing into the fluorescent light emitting layer, or adjusting a carrier balance, in the case where the fluorescent light emitting layers and the phosphorescent light emitting layers are stacked. The space layer can also be provided among the plurality of phosphorescent light emitting layers.

Since the space layer is provided between the light emitting layers, a material having both an electron transporting capability and a hole transporting capability is preferred. Also, one having a triplet energy of 2.6 eV or more is preferred in order to prevent triplet energy diffusion in the adjacent phosphorescent light emitting layer. Examples of the material used for the space layer include the same as those used for the hole transporting layer as described above.

### Blocking Layer

The blocking layer such as the electron blocking layer, the hole blocking layer, or the exciton blocking layer may be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function of preventing excitons generated in the light emitting layer from diffusing into the surrounding layers, and trapping the excitons within the light emitting layer.

Each layer of the organic EL device may be formed by a conventionally known vapor deposition method, a coating method, or the like. For example, formation can be performed by a known method using a vapor deposition method such as a vacuum vapor deposition method, or a molecular beam vapor deposition method (MBE method), or a coating method using a solution of a compound for forming a layer, such as a dipping method, a spin-coating method, a casting method, a bar-coating method, and a roll-coating method.

The film thickness of each layer is not particularly limited, but is typically 5 nm to 10 μm, and more preferably 10 nm to 0.2 μm because in general, in the case where the film thickness is too small, defects such as pinholes are likely to occur, and in the case where the film thickness is too large, on the other hand, a high driving voltage is required and the efficiency decreases.

In the organic EL device having the hole transporting layer having the two-layer structure or the three-layer structure of the present invention, the total thickness of the first hole transporting layer and the second hole transporting layer is preferably 30 nm or more and 150 nm or less, and more preferably 40 nm or more and 130 nm or less.

In one embodiment of the present invention, the thickness of the second hole transporting layer having the two-layer structure or the three-layer structure is preferably 5 nm or more, more preferably 20 nm or more, further preferably 25 nm or more, and particularly preferably 35 nm or more, and is preferably 100 nm or less.

In one embodiment of the present invention, the thickness of the hole transporting layer adjacent to the light emitting layer is preferably 5 nm or more, more preferably 20 nm or more, further preferably 25 nm or more, and particularly preferably 30 nm or more, and is preferably 100 nm or less.

In the organic EL device having the hole transporting layer having the two-layer structure or the three-layer structure of the present invention, the ratio of the film thickness D2 of the second hole transporting layer and the film thickness D1 of the first hole transporting layer is preferably 0.3<D2/D1<4.0, more preferably 0.5<D2/D1<3.5, and further preferably 0.75<D2/D1<3.0.

Preferred embodiments of the organic EL device of the present invention include the following:

(1) Organic EL device having hole transporting layer having two-layer structure
a first embodiment in which the second hole transporting layer contains the inventive compound, and the first hole transporting layer does not contain the inventive compound;
a second embodiment in which both the first hole transporting layer and the second hole transporting layer contain the inventive compound;
a third embodiment in which the first hole transporting layer contains the inventive compound, and the second hole transporting layer does not contain the inventive compound;
   (2) Organic EL device having hole transporting layer having three-layer structure
a fourth embodiment in which the first hole transporting layer contains the inventive compound, and the second and third hole transporting layers do not contain the inventive compound;
a fifth embodiment in which the second hole transporting layer contains the inventive compound, and the first and third hole transporting layers do not contain the inventive compound;
a sixth embodiment in which the third hole transporting layer contains the inventive compound, and the first and second hole transporting layers do not contain the inventive compound;
a seventh embodiment in which the first and second hole transporting layers contain the inventive compound, and the third hole transporting layer does not contain the inventive compound;
an eighth embodiment in which the first and third hole transporting layers contain the inventive compound, and the second hole transporting layer does not contain the inventive compound;
a ninth embodiment in which the second and third hole transporting layers contain the inventive compound, and the first hole transporting layer does not contain the inventive compound; and
a tenth embodiment in which all the first to third hole transporting layers contain the inventive compound.

### Electronic Device

The organic EL device according to one embodiment of the present invention can be used for an electronic device, such as a display device and a light emitting device. Examples of the display device include a display component of an organic EL panel module and the like, a television set, a mobile phone, a tablet, and a personal computer. Examples of the light emitting device include a lighting device and an automobile lamp.

The organic EL device can be used for an electronic device, for example, a display component of an organic EL panel module and the like, a display device, such as a television set, a mobile phone, a personal computer, and a light emitting device, such as a lighting device and an automobile lamp.

### EXAMPLES

The present invention will be described in more detail with reference to examples, but the present invention is not limited to the examples shown below.

### Inventive Compounds Used in Production of Organic EL Devices of Examples 1 and 2

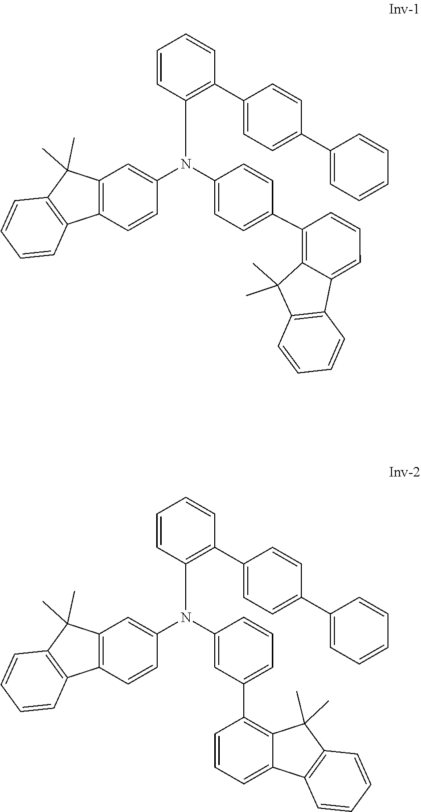 [SMILES: CC1(C)c2ccccc2-c2ccc(N(c3ccc(-c4cccc5c4C(C)(C)c4ccccc4-5)cc3)c3ccccc3-c3ccc(-c4ccccc4)cc3)cc21 ; CC1(C)c2ccccc2-c2ccc(N(c3cccc(-c4cccc5c4C(C)(C)c4ccccc4-5)c3)c3ccccc3-c3ccc(-c4ccccc4)cc3)cc21]

### Comparative Compound Used in Production of Organic EL Device of Comparative Examples 1

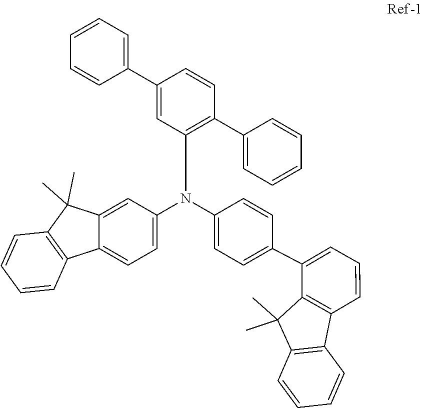 [SMILES: CC1(C)c2ccccc2-c2ccc(N(c3ccc(-c4cccc5c4C(C)(C)c4ccccc4-5)cc3)c3cc(-c4ccccc4)ccc3-c3ccccc3)cc21]

### Other Compounds Used in Production of Organic EL Devices of Examples 1 and 2 and Comparative Example 1

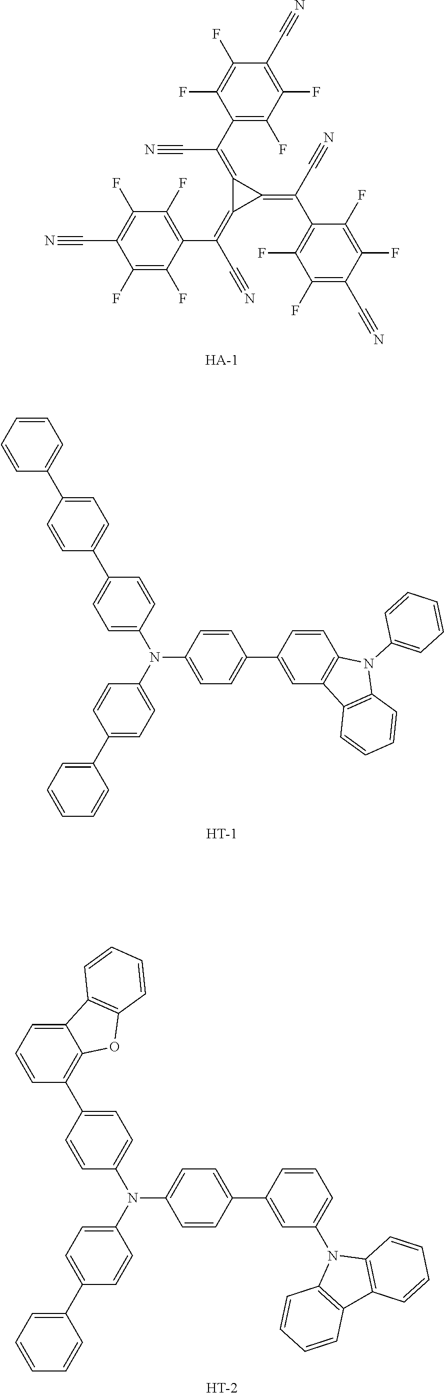

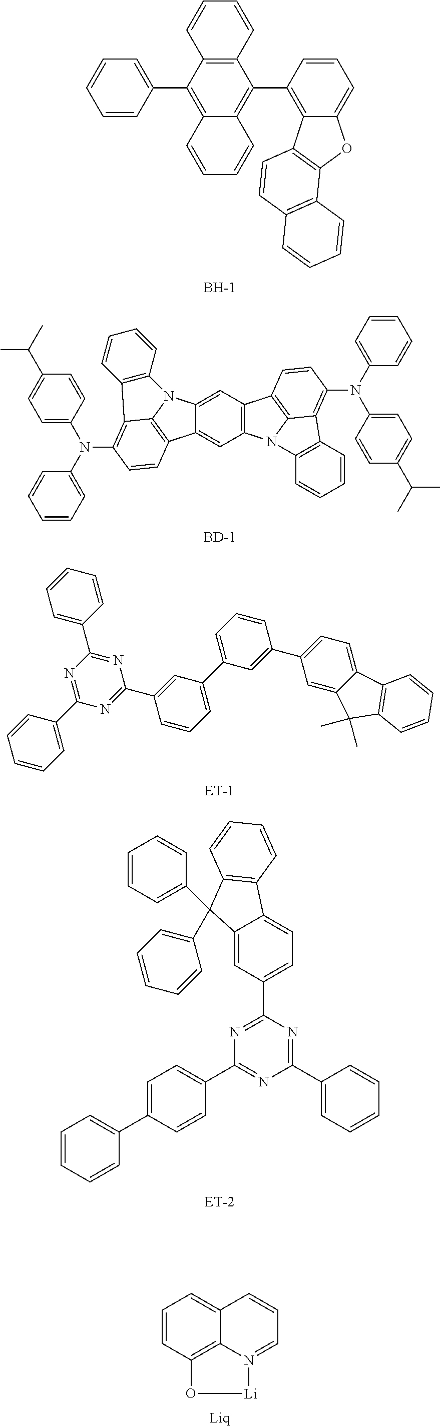

### Production of Organic EL Device

### Example 1

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with an ITO transparent electrode was mounted on a substrate holder of a vacuum vapor deposition apparatus, and firstly, Compound HT-1 and Compound HA-1 were vapor co-deposited on the surface having the transparent electrode formed thereon, so as to cover the transparent electrode, resulting in a hole injecting layer having a film thickness of 10 nm. The mass ratio of Compound HT-1 and Compound HA-1 (HT-1/HA-1) was 97/3.

On the hole injecting layer, Compound HT-1 was then vapor deposited to form a first hole transporting layer having a film thickness of 40 nm.

On the first hole transporting layer, the inventive compound Inv-1 was then vapor deposited to form a second hole transporting layer having a film thickness of 40 nm.

On the second hole transporting layer, the compound HT-2 was then vapor deposited to form a third hole transporting layer having a film thickness of 5 nm.

On the third hole transporting layer, Compound BH-1 (host material) and Compound BD-1 (dopant material) were then vapor co-deposited to form a light emitting layer having a film thickness of 20 nm. The mass ratio of Compound BH-1 and Compound BD-1 (BH-1/BD-1) was 99/1.

On the light emitting layer. Compound ET-1 was then vapor deposited to form a first electron transporting layer having a film thickness of 5 nm.

On the first electron transporting layer, Compound ET-2 and Liq were then vapor co-deposited to form a second electron transporting layer having a film thickness of 25 nm. The mass ratio of Compound ET-2 and Liq (ET-2/Liq) was 50/50.

On the second electron transporting layer, Yb was then vapor deposited to form an electron injecting electrode having a film thickness of 1 nm.

Finally, on the electron injecting electrode, metal Al was then vapor deposited to form a metal cathode having a film thickness of 50 nm.

The layer configuration of the organic EL device of Example 1 thus obtained is shown below.
ITO (130)/HT-1/HA-1=97/3 (10)/HT-1 (40)/Inv-1 (40)/HT-2 (5)/BH-1/BD-1=99/1 (20)/ET-1 (5)/ET-2/Liq=50/50(25)/Yb (1)/Al (50)

In the layer configuration, the numerals in parentheses each indicate the film thickness (nm), and the ratios each are a mass ratio.

### Example 2 and Comparative Example 1

Organic EL devices were produced in the same manner as in Example 1 except that the inventive compound Inv-2 was used instead of the inventive compound Inv-1 in Example 2, and the comparative compound Ref-1 was used instead of the inventive compound Inv-1 in Comparative Example 1.

### Evaluation of Organic EL Device

### (1) Measurement of External Quantum Efficiency (EQE)

The resulting organic EL device was driven with a direct current at a constant current with a current density of 10 mA/cm² under room temperature. The luminance thereof was measured with a luminance meter (Spectroradiometer CS-1000, available from Konica Minolta Japan, Inc.), and the external quantum efficiency (%) was obtained from the result. The results are shown in Table 1.

TABLE 1

| | Material for | |
|---|---|---|
| | second hole | EQE |
| | transporting | (%) at |
| | layer | 10 mA/cm² |
| Example 1 | Inv-1 | 11.6 |
| Example 2 | Inv-2 | 11.6 |
| Comparative | Ref-1 | 10.3 |
| Example 1 | | |

It is apparent from the results shown in Table 1 that the inventive compounds Inv-1 and Inv-2 provide organic EL devices having a higher external quantum efficiency than the comparative compound Ref-1.

### Inventive Compounds Synthesized in Synthesis Examples

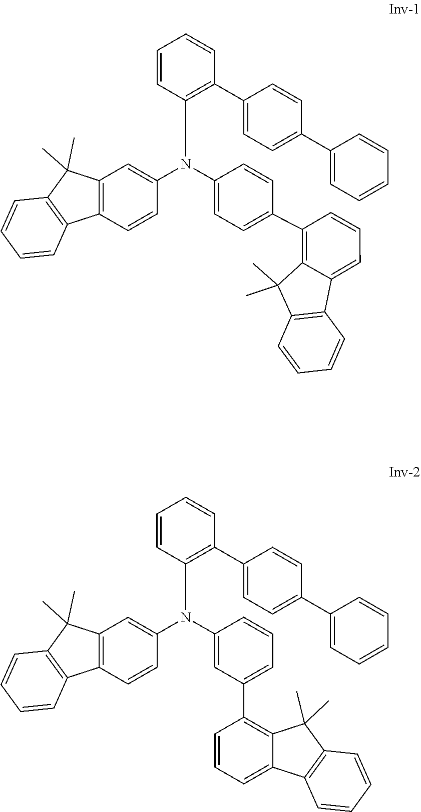 [SMILES: CC1(C)c2ccccc2-c2ccc(N(c3ccc(-c4cccc5c4C(C)(C)c4ccccc4-5)cc3)c3ccccc3-c3ccc(-c4ccccc4)cc3)cc21 ; CC1(C)c2ccccc2-c2ccc(N(c3cccc(-c4cccc5c4C(C)(C)c4ccccc4-5)c3)c3ccccc3-c3ccc(-c4ccccc4)cc3)cc21]

### Intermediate Synthesis Example 1

### Synthesis of Intermediate A

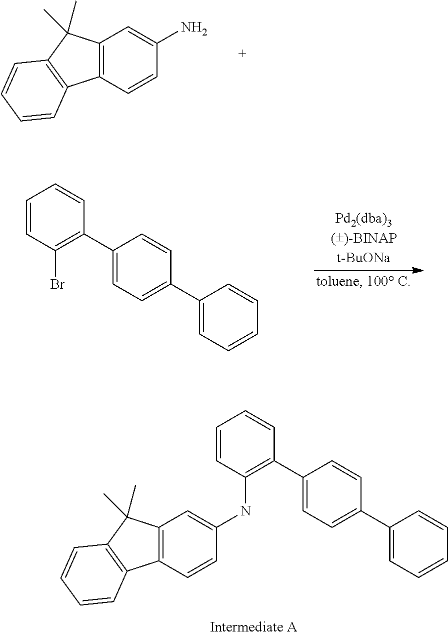 [SMILES: CC1(C)c2ccccc2-c2ccc(N)cc21 ; Brc1ccccc1-c1ccc(-c2ccccc2)cc1 ; CC1(C)c2ccccc2-c2ccc(Nc3ccccc3-c3ccc(-c4ccccc4)cc3)cc21]

Under an argon atmosphere, a mixture of 9,9-dimethyl-9H-fluorene-2-amine (13.19 g, 63.00 mmol), 2-bromo-1,1′:4′,1″-terphenyl (18.55 g, 60.00 mmol), tris(dibenzylideneacetone) dipalladium(0) (1.10 g, 1.200 mmol), (+)-2,2′-bis(diphenylphosphino)-1,1′-binaphthyl (1.50 g, 2.40 mmol), sodium t-butoxide (6.92 g, 72.00 mmol), and toluene (200 mL) was agitated at 100° C. for 2 hours. The reaction liquid was cooled to room temperature, and then concentrated under reduced pressure. The resulting residue was purified through silica gel column chromatography, so as to provide an intermediate A (20.9 g) as a pale yellow solid matter. The yield was 45%.

### Synthesis Example 1

### Synthesis of Inventive Compound Inv-1

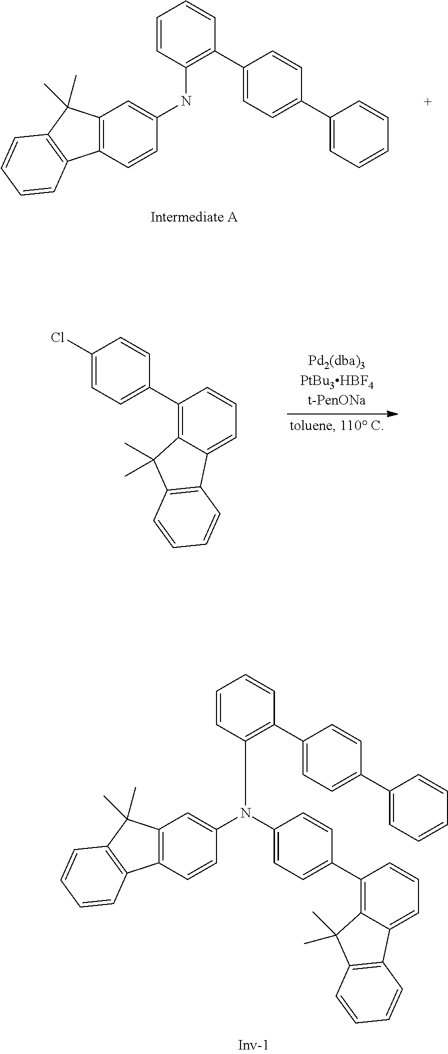 [SMILES: CC1(C)c2ccccc2-c2ccc(Nc3ccccc3-c3ccc(-c4ccccc4)cc3)cc21 ; CC1(C)c2ccccc2-c2cccc(-c3ccc(Cl)cc3)c21 ; CC1(C)c2ccccc2-c2ccc(N(c3ccc(-c4cccc5c4C(C)(C)c4ccccc4-5)cc3)c3ccccc3-c3ccc(-c4ccccc4)cc3)cc21]

Under an argon atmosphere, a mixture of the intermediate A (4.38 g, 10.00 mmol), 1-(4-chlorophenyl)-9,9-dimethyl-9H-fluorene (3.20 g, 10.50 mmol), tris(dibenzylideneacetone) dipalladium(0) (0.183 g, 0.200 mmol), tri-t-butylphosphonium tetrafluoroborate (0.232 g, 0.800 mmol), sodium t-pentoxide (3.85 g, 14.00 mmol), and toluene (66.7 mL) was agitated at 110° C. for 4 hours. The reaction liquid was cooled to room temperature, and then concentrated under reduced pressure. The resulting residue was purified through silica gel column chromatography and recrystallization, so as to provide 5.55 g of a white solid matter. The yield was 79%.

As a result of the mass spectrum analysis, the resulting matter was the inventive compound Inv-1 and exhibited m/e=705 with respect to the molecular weight of 705.34.

### Synthesis Example 2

### Synthesis of Inventive Compound Inv-2

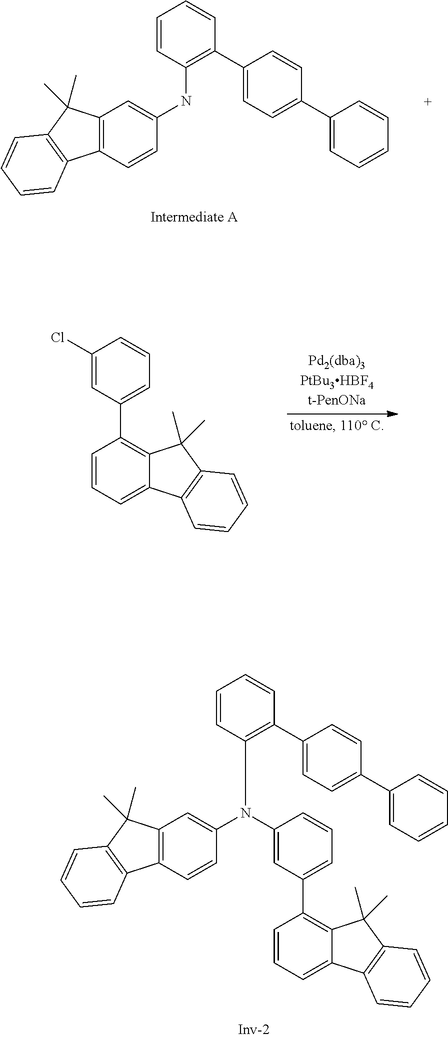 [SMILES: CC1(C)c2ccccc2-c2ccc(Nc3ccccc3-c3ccc(-c4ccccc4)cc3)cc21 ; CC1(C)c2ccccc2-c2cccc(-c3cccc(Cl)c3)c21 ; CC1(C)c2ccccc2-c2ccc(N(c3cccc(-c4cccc5c4C(C)(C)c4ccccc4-5)c3)c3ccccc3-c3ccc(-c4ccccc4)cc3)cc21]

The same procedure as in Synthesis Example 1 was performed except that 1-(3-chlorophenyl)-9,9-dimethyl-9H-fluorene was used instead of 1-(4-chlorophenyl)-9,9-dimethyl-9H-fluorene used in Synthesis Example 1, so as to provide 5.76 g of a white solid matter. The yield was 82%.

As a result of the mass spectrum analysis, the resulting matter was the inventive compound Inv-2 and exhibited m/e=705 with respect to the molecular weight of 705.34.

### REFERENCE SIGN LIST

1, 11, 12: Organic EL device
2: Substrate
3: Anode
4: Cathode
5: Light emitting layer
5a: First light emitting layer
5b: Second light emitting layer
6: Hole transporting zone (hole transporting layer)
6a: Hole injecting layer
6b: First hole transporting layer
6c: Second hole transporting layer
6d: Third hole transporting layer
7: Electron transporting zone (electron transporting layer)
7a: First electron transporting layer
7b: Second electron transporting layer
10, 20, 30: Light emitting unit

1. A compound represented by the following formula (1): 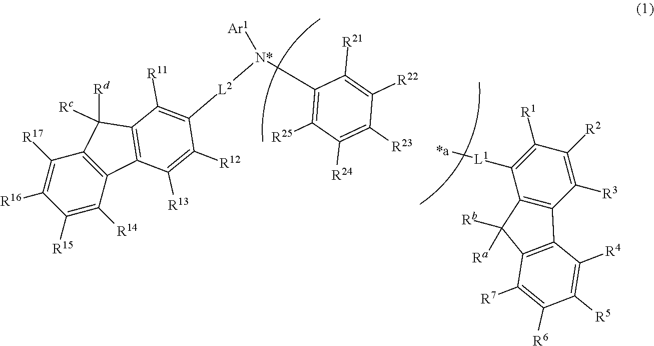
wherein in the formula (1),
N* represents a center nitrogen atom,
L¹ and L² each independently represent a single bond or a substituted or unsubstituted phenylene group, in which the substituent is an unsubstituted alkyl group having 1 to 10 carbon atoms, and no ring is condensed to the phenylene group,
R¹ to R⁷ and R¹¹ to R¹⁷ each independently represent a hydrogen atom or an unsubstituted alkyl group having 1 to 10 carbon atoms,
adjacent two selected from R¹ to R⁷ and R¹¹ to R¹⁷ are not bonded to each other, and therefore do not form a ring,
R^{a} and R^{b} each independently represent a hydrogen atom or a substituted or unsubstituted alkyl group having 1 to 30 carbon atoms,
R^{a} and R^{b} may be bonded to each other to form a substituted or unsubstituted ring,
R^{c} and R^{d} each independently represent a hydrogen atom, a substituted or unsubstituted alkyl group having 1 to 30 carbon atoms, or a substituted or unsubstituted aryl group having 6 to 30 ring carbon atoms,
R^{c} and R^{d} may be bonded to each other to form a substituted or unsubstituted ring,
provided that in the case where R^{C} and R^{d} are not bonded to each other not to form a substituted or unsubstituted ring, at least one of R^{c} and R^{d} represents a substituted or unsubstituted alkyl group having 1 to 30 carbon atoms,
R^{c} and R^{d} are not bonded to each other not to form a substituted or unsubstituted fluorene ring with a carbon atom at the 9-position of the fluorene skeleton,
one selected from R²¹ to R²⁵ represents a single bond bonded to *a, and R²¹ to R²⁵ that are not the single bond bonded to *a represent hydrogen atoms, and
Ar¹ represents a group represented by any of the following formulae (1a) to (1f): 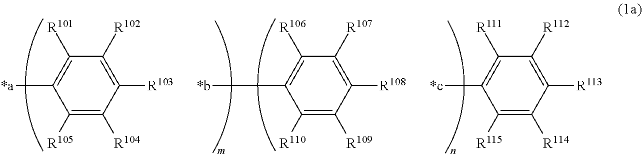
wherein in the formula (1a),
** represents a bonding site to the center nitrogen atom N*,
R¹⁰¹ to R¹⁰⁵ and R¹⁰⁶ to R¹¹⁰ each independently represent a hydrogen atom or an unsubstituted alkyl group having 1 to 6 carbon atoms,
provided that one selected from R¹⁰¹ to R¹⁰⁵ represents a single bond bonded to *b, and one selected from R¹⁰⁶ to R¹¹⁰ represents a single bond bonded to *c,
adjacent two selected from R¹⁰¹ to R¹⁰⁵ that are not the single bond are not bonded to each other, and therefore do not form a ring, and
adjacent two selected from R¹⁰⁶ to R¹¹⁰ that are not the single bond are not bonded to each other, and therefore do not form a ring,
m represents 0, 1, or 2, n represents 0, 1, or 2,
in which m+n is 0, 2, or 3,
R¹¹¹ to R¹¹⁵ each independently represent a hydrogen atom, a substituted or unsubstituted alkyl group having 1 to 6 carbon atoms, or a substituted or unsubstituted heteroaryl group having 5 to 13 ring atoms, and
adjacent two selected from R¹¹¹ to R¹¹⁵ may be bonded to each other to form one or multiple unsubstituted benzene rings, or may not be bonded to each other, and therefore may not form a ring, 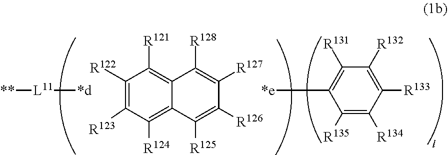
wherein in the formula (1b),
** represents a bonding site to the center nitrogen atom N*,
L¹¹ represents a single bond, an unsubstituted arylene group having 6 to 12 ring carbon atoms, or an unsubstituted divalent heterocyclic group having 5 to 13 ring atoms,
R¹²¹ to R¹²⁸ each independently represent a hydrogen atom, a substituted or unsubstituted alkyl group having 1 to 6 carbon atoms, or a substituted or unsubstituted aryl group having 6 to 12 ring carbon atoms,
provided that one selected from R¹²¹ to R¹²³ represents a single bond bonded to *d, the other one selected from R¹²¹ to R¹²⁸ represents a single bond bonded to *e, and
adjacent two selected from R¹²¹ to R¹²⁸ that are not the single bond bonded to *d and a single bond bonded to *e are not bonded to each other, and therefore do not form a ring,
R¹³¹ to R¹³⁵ each independently represent a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
adjacent two selected from R¹³¹ to R¹³⁵ may be bonded to each other to form one or multiple unsubstituted benzene rings, or may not be bonded to each other, and therefore may not form a ring, and
l represents 0 or 1, 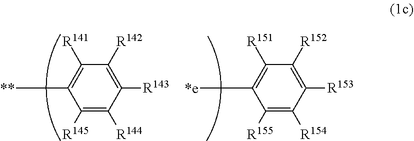
wherein in the formula (1c),
** represents a bonding site to the center nitrogen atom N*,
R¹⁴¹ to R¹⁴⁵ each independently represent a hydrogen atom or a substituted or unsubstituted alkyl group having 1 to 6 carbon atoms,
provided that one selected from R¹⁴¹, R¹⁴², R¹⁴⁴, and R¹⁴⁵ represents a single bond bonded to *e, and
adjacent two selected from R¹⁴³, and R¹⁴¹, R¹⁴², R¹⁴⁴, and R¹⁴⁵ that are not the single bond bonded to *e are not bonded to each other, and therefore do not form a ring,
R¹⁵¹ to R¹⁵⁵ each independently represent a hydrogen atom or a substituted or unsubstituted alkyl group having 1 to 6 carbon atoms, and
adjacent two selected from R¹⁵¹ to R¹⁵⁵ are not bonded to each other, and therefore do not form a ring, 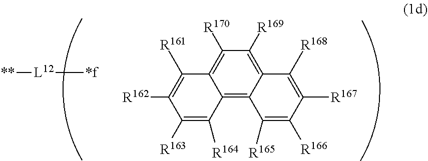
wherein in the formula (1d),
** represents a bonding site to the center nitrogen atom N*,
L¹² represents a single bond, an unsubstituted arylene group having 6 to 12 ring carbon atoms, or an unsubstituted divalent heterocyclic group having 5 to 13 ring atoms,
R¹⁶¹ to R¹⁷⁰ each independently represent a hydrogen atom, a substituted or unsubstituted alkyl group having 1 to 6 carbon atoms, or a substituted or unsubstituted aryl group having 6 to 12 ring carbon atoms,
provided that in the case where L¹² represents a single bond, one selected from R¹⁶¹ to R¹⁶⁸ represents a single bond bonded to *f, and in the case where L¹² represents an unsubstituted arylene group having 6 to 12 ring carbon atoms or an unsubstituted divalent heterocyclic group having 5 to 13 ring atoms, one selected from R¹⁶¹ to R¹⁷⁰ represents a single bond bonded to *f, and
adjacent two selected from R¹⁶¹ to R¹⁷⁰ that are not the single bond are not bonded to each other, and therefore do not form a ring, 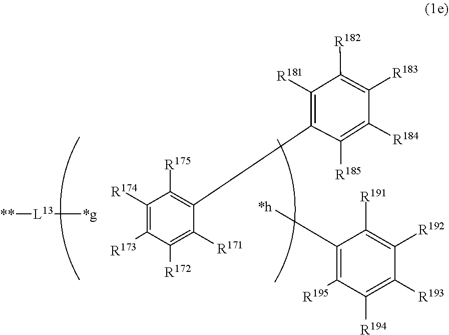
wherein in the formula (1e),
** represents a bonding site to the center nitrogen atom N*,
L¹³ represents a single bond, an unsubstituted arylene group having 6 to 12 ring carbon atoms, or an unsubstituted divalent heterocyclic group having 5 to 13 ring atoms,
R¹⁷¹ to R¹⁷⁵ each independently represent a hydrogen atom, an unsubstituted alkyl group having 1 to 6 carbon atoms, or an unsubstituted phenyl group,
provided that one selected from R¹⁷¹ to R¹⁷⁵ represents a single bond bonded to *g, and the other one selected from R¹⁷¹ and R¹⁷² represents a single bond bonded to *h, and
adjacent two selected from R¹⁷¹ to R¹⁷⁵ that are not the single bond bonded to *g and the single bond bonded to *h are not bonded to each other, and therefore do not form a ring,
R¹⁸¹ to R¹⁸⁵ and R¹⁹¹ to R¹⁹⁵ each independently represent a hydrogen atom or an unsubstituted alkyl group having 1 to 6 carbon atoms, and
adjacent two selected from R¹⁸¹ to R¹⁸⁵ and R¹⁹¹ to R¹⁹⁵ may be bonded to each other to form one or multiple unsubstituted benzene rings, or may not be bonded to each other, and therefore may not form a ring, 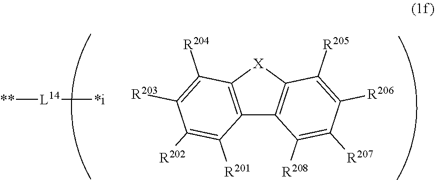
wherein in the formula (1f),
** represents a bonding site to the center nitrogen atom N*,
L¹⁴ represents a single bond or an unsubstituted phenylene group,
X represents an oxygen atom, a sulfur atom, or CR^{A}R^{B},
wherein R^{A} and R^{B} each independently represent a hydrogen atom, a substituted or unsubstituted alkyl group having 1 to 30 carbon atoms, or a substituted or unsubstituted aryl group having 6 to 30 ring carbon atoms, and
R^{A} and R^{B} may be bonded to each other to form a substituted or unsubstituted ring, or may not be bonded to each other, and therefore may not form a ring, and
R²⁰¹ to R²⁰⁸ each independently represent a hydrogen atom, a substituted or unsubstituted alkyl group having 1 to 6 carbon atoms, or a substituted or unsubstituted aryl group having 6 to 12 ring carbon atoms,
provided that in the case where L¹⁴ represents a phenylene group, and X represents an oxygen atom, one selected from R²⁰¹ to R²⁰³, R²⁰⁶ to R²⁰⁸, R^{A}, and R^{B} represents a single bond bonded to *i, and
in the case where L¹⁴ represents a single bond, and X represents an oxygen atom, a sulfur atom, or CR^{A}R^{B}, and in the case where L¹⁴ represents a phenylene group, and X represents a sulfur atom or CR^{A}R^{B}, one selected from R²⁰¹ to R²⁰⁸, R^{A}, and R^{B} represents a single bond bonded to *i,
in the case where X represents CR^{A}R^{B}, adjacent two selected from R²⁰¹ to R²⁰⁸ that are not the single bond are not bonded to each other, and therefore do not form a ring, and
in the case where X represents an oxygen atom or a sulfur atom, adjacent two selected from R²⁰¹ to R²⁰⁸ that are not the single bond may be bonded to each other to form one or multiple unsubstituted benzene rings, or may not be bonded to each other, and therefore may not form a ring.

2. The compound according to claim 1, wherein Ar¹ represents a group represented by any one of the formula (1a) or (1f).

3. The compound according to claim 1, wherein in the formula (1a), one selected from R¹⁰¹ to R¹⁰⁵ represents a single bond bonded to *b, or one selected from R¹⁰⁶ to R¹¹⁰ represents a single bond bonded to *c.

4. The compound according to claim 1, wherein X represents CR^{A}R^{B}.

5. The compound according to claim 1, wherein one or both of L¹ and L² represents a single bond.

6. The compound according to claim 1, wherein one selected from R²² to R²⁴ represents a single bond bonded to *a.

7. The compound according to claim 1, wherein R^{a} and R^{b} each represent a methyl group.

8. The compound according to claim 1, wherein R^{c} and R^{d} each represent a substituted or unsubstituted alkyl group having 1 to 30 carbon atoms.

9. The compound according to claim 1, wherein R^{c} and R^{d} each represent a methyl group.

10. The compound according to claim 1, wherein R^{c} and R^{d} are bonded to each other to form a substituted or unsubstituted spiro ring with a carbon atom at the 9-position of the fluorene skeleton, and the spiro ring is selected from the following: 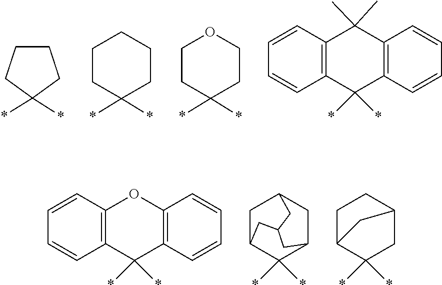
wherein * represents a bonding site to the benzene ring of the fluorene skeleton.

11. The compound according to claim 1, wherein the compound contains at least one deuterium atom.

12. A material for an organic electroluminescent device, comprising the compound according to claim 1.

13. A material for a hole transporting layer, comprising the compound according to claim 1.

14. An organic electroluminescent device comprising an anode, a cathode, and organic layers intervening between the anode and the cathode, the organic layers including a light emitting layer, at least one layer of the organic layers containing the compound according to claim 1.

15. The organic electroluminescent device according to claim 14, wherein the organic layers include a hole transporting zone intervening between the anode and the light emitting layer, and the hole transporting zone contains the compound.

16. The organic electroluminescent device according to claim 15, wherein the hole transporting zone includes a first hole transporting layer on the anode side and a second hole transporting layer on the cathode side, and one or both of the first hole transporting layer and the second hole transporting layer contains the compound.

17. The organic electroluminescent device according to claim 16, wherein the light emitting layer and the second hole transporting layer are directly in contact with each other.

18. The organic electroluminescent device according to claim 16, wherein the organic electroluminescent device has a total thickness of the first hole transporting layer and the second hole transporting layer of 30 nm or more and 150 nm or less.

19. The organic electroluminescent device according to claim 15, wherein the hole transporting zone includes a first hole transporting layer, a second hole transporting layer, and a third hole transporting layer in this order from the anode side, and only one layer selected from the first to third hole transporting layers, only two layers selected from the first to third hole transporting layers, or all the first to third hole transporting layers contain the compound.

20. The organic electroluminescent device according to claim 19, wherein the light emitting layer and the third hole transporting layer are directly in contact with each other.

21. The organic electroluminescent device according to claim 19, wherein the organic electroluminescent device has a total thickness of the first hole transporting layer and the second hole transporting layer of 30 nm or more and 150 nm or less.

22. The organic electroluminescent device according to claim 14, wherein the light emitting layer is a single layer.

23. The organic electroluminescent device according to claim 14, wherein the light emitting layer contains a light emitting compound exhibiting fluorescent light emission having a main peak wavelength of 500 nm or less.

24. The organic electroluminescent device according to claim 14, wherein the light emitting layer contains a fluorescent dopant material.

25. An electronic device comprising the organic electroluminescent device according to claim 14.
